(19) United States
(12) Patent Application Publication
(10) Pub. No.: US 2023/0083012 A1
(43) Pub. Date: Mar. 16, 2023

(54) DIHYDROPYRIMIDINE DERIVATIVES AND USES THEREOF IN THE TREATMENT OF HBV INFECTION OR OF HBV-INDUCED DISEASES

(71) Applicant: Janssen Sciences Ireland Unlimited Company, Co Cork (IE)

(72) Inventors: Yimin JIANG, Londonderry, NH (US); Zhanling CHENG, Shanghai (CN); Gang DENG, Shanghai (CN); Zhiguo LIU, Shanghai (CN); Chao LIANG, Shanghai (CN); Jianping WU, Shanghai (CN); Linglong KONG, Shanghai (CN); Xiangjun DENG, Shanghai (CN); Yanping XU, Noblesville, IN (US)

(21) Appl. No.: 17/597,891

(22) PCT Filed: Jul. 30, 2020
(86) PCT No.: PCT/CN2020/105764

§ 371 Date: Jan. 28, 2022

(30) Foreign Application Priority Data
Jul. 31, 2019 (CN) PCT/CN2019/098569
Feb. 28, 2020 (CN) PCT/CN2020/077163
Jun. 18, 2020 (CN) PCT/CN2020/096777

(51) Int. Cl. C07D 487/04; C07D 417/14; C07D 498/04; A61K 45/06

(52) U.S. Cl. CPC C07D 487/04; C07D 417/14; C07B 2200/05; A61K 45/06; C07D 498/04

(57) ABSTRACT

Provided are dihydropyrimidine derivatives which are useful in the treatment or prevention of HBV infection or of HBV-induced diseases, more particularly of HBV chronic infection or of diseases induced by HBV chronic infection, as well as pharmaceutical or medical applications thereof.

### BACKGROUND

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant worldwide medical problem, due to suboptimal treatment options and sustained rates of new infections in most parts of the developing world.

Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress.

Capsid structures also respond to environmental cues to allow un-coating after viral entry. Consistently, the appropriate timing of capsid assembly and dis-assembly, the appropriate capsid stability and the function of core protein have been found to be critical for viral infectivity.

Background references on dihydropyrimidine derivatives in the treatment of HBV infection include WO 2014/029193, CN103664899, CN103664925, and CN103664897.

There is a need in the art for therapeutic agents that can increase the suppression of virus production and that can treat, ameliorate, or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

### SUMMARY

Provided, in one aspect, is a compound of Formula (I)

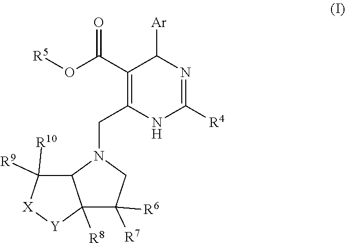

including the deuterated, stereoisomeric or tautomeric forms thereof, wherein:

Ar is selected from the group consisting of phenyl, thiophenyl and pyridyl, optionally substituted with one or more substituents selected from the group consisting of C₁₋₄alkyl, hydroxyl, halogen, and CN;

R⁴ is selected from the group consisting of thiazolyl, imidazolyl, oxazolyl and pyridyl, each of which may be optionally substituted with one or more substituents, each independently selected from methyl or halo;

R⁵ is C₁₋₄alkyl;

R⁶, R⁷ and R⁸ are each independently selected from the group consisting of H and halo;

R⁹ and R¹⁰ are each independently selected from the group consisting of H, halo and OH; or

R⁹ and R¹⁰, together with the carbon atom to which they are attached, form C(═O);

X is selected from the group consisting of CH₂, C(═O), O, S, S(═O), S(═O)₂, NH, NR^{11a}, CHR^{12a}, and CR¹⁵R¹⁶; and

Y is selected from the group consisting of CH₂, C(═O), O, NH, NR^{11b}, and CHR^{12b};

wherein R^{11a}, R^{11b}, R^{12a}, and R^{12b} are each independently selected from the group consisting of —CN; —C₁₋₆alkyl, —COOR^{x}; —C₁₋₉alkyl-COOR^{x}; —C₁₋₆alkyl-O—C₁₋₆alkyl-COOR^{x}; -Cy-COOR^{x}; —C₁₋₆alkyl-C(═O)—NR^{c}—S(═O)₂—C₁₋₆alkyl; —C₁₋₆alkyl-Cy-COOR^{x}; -Cy-C₁₋₆alkyl-COOR^{x}; —C₁₋₆ alkyl-Cy-C₁₋₆alkyl-COOR^{x}; —C(═O)—C₁₋₆alkyl; —C(═O)—C₁₋₆alkyl-COOR^{x}; —C(═O)—Cy-COOR^{x}; —C(═O)—O—C₁₋₆alkyl-COOR^{x}; —C(═O)—C₁₋₆alkyl-O—C₁₋₆alkyl-COOR^{x}; —C(═O)H; —C(═O)—NR^{a}R^{b}; —C(═O)-Het¹; —C(═O)—Cy; —C(═O)—NR^{c}—C₁₋₆alkyl-COOR^{x}; —C(═O)—C₁₋₆alkyl-NR^{c}—C₁₋₆alkyl-COOR^{x}; —C(═O)—NR^{c}—COOR^{x}; —C(═O)—NR^{c}—CO—NR^{a}R^{b}; —C(═O)—NR-Cy-COOR^{x}; —C(═O)—NR^{c}—S(═O)₂—C₁₋₆alkyl; —C(═O)-Het¹-COOR^{x}; —C(═O)—NR^{c}-Het¹-COOR^{x}; —C(═O)—C(═O)—NR^{a}R^{b}; —C(═O)—C(═O)-Het¹; —C(═O)—C(═O)—O—C₂₋₆alkenyl; -Het¹-COOR^{x}; -Het¹-C₁₋₆alkyl-COOR^{x}; —C₁₋₆alkyl-Het¹-COOR^{x}; —C₁₋₆alkyl-Het¹-C₁₋₆alkyl-COOR^{x}; —C₁₋₆alkyl-C(═O)-Het¹-COOR^{x}; -Het²-COOR^{x}; —C₁₋₆alkyl-Het²; —C₁₋₆alkyl-Het²-COOR^{x}; -Het²-C₁₋₆alkyl-COOR^{x}; —C₁₋₆alkyl-Het²-C₁₋₆alkyl-COOR^{x}; —NR^{c}—C₁₋₆alkyl-COOR^{x}; —NR^{c}—Cy-COOR^{x}; —NR^{c}—Het¹-COOR^{x}; —O—C₁₋₉alkyl-COOR^{x}; —S(═O)₂—NR^{a}R^{b}; —S(═O)₂—C₁₋₆alkyl; —S(═O)₂—C₁₋₆alkyl-COOR^{x}; —S(═O)₂—Cy-COOR^{x}; —S(═O)₂—Cy-C₁₋₆alkyl-COOR^{x}; —S(═O)₂—NR^{c}-Cy-COOR^{x}; —S(═O)₂—NR^{c}-Het²; —S(═O)₂-Het¹-COOR^{x}; —S(═O)₂-Het¹-C₁₋₆alkyl-COOR^{x}; —S(═O)₂—NR^{c}—C(═O)—C₁₋₆alkyl; —C(═O)—NR^{c}—S(═O)₂—C₁₋₆alkyl; and —C₁₋₆alkyl-C(═O)—NR^{c}—S(═O)₂—C₁₋₆alkyl; wherein

R^{a}, R^{b} and R^{c} are each independently selected from H and —C₁₋₄alkyl;

at each instance, C₁₋₆alkyl and C₁₋₉alkyl may be optionally substituted with one or more substituents, each independently selected from halo and hydroxyl;

R^{x} is selected from H and —C₁₋₆alkyl;

Cy is selected from C₃₋₇cycloalkyl, 5- to 11-membered bicyclic saturated carbocyclyl, each optionally substituted with one or more substituents selected from halo and —C₁₋₄alkyl;

Het¹ represents a 4- to 8-membered saturated ring in which 1 or 2 of the ring members is a heteroatom each independently selected from the group consisting of N, O, and S; wherein the 4- to 8-membered saturated ring may be optionally substituted with one or more substituents, each independently selected from C₁₋₄alkyl and OH; and

Het² represents a 5- to 6-membered aromatic ring in which 1, 2, 3 or 4 of the ring members is a heteroatom each independently selected from N, O, or S; wherein the 5- to 6-membered aromatic ring is optionally substituted with one or more substituents, each independently selected from C₁₋₄alkyl and halo;

with the proviso that CR⁹R¹⁰ and X, or X and Y, are not simultaneously both C(═O);

R¹⁵ and R¹⁶, together with the carbon atom to which they are attached, form a C₃₋₇cycloalkyl, optionally substituted with one or more substituents selected from halo and —C₁₋₄alkyl; or a pharmaceutically acceptable salt or a solvate thereof.

In another aspect, provided herein is a pharmaceutical composition comprising at least one compound of Formula (I), or a pharmaceutically acceptable salt thereof, together with a pharmaceutically acceptable carrier.

In another aspect, provided herein is a pharmaceutical composition comprising at least one disclosed compound, together with a pharmaceutically acceptable carrier. In another aspect, provided herein is a method of treating an HBV infection in an individual in need thereof, comprising administering to the individual a therapeutically effective amount of a compound of Formula (I) or a pharmaceutically acceptable salt thereof. In another aspect, provided herein is any of the compounds described herein, or the pharmaceutical composition of the invention, for use as a medicament. In a further aspect, provided herein is any of the compounds described herein, or the pharmaceutical composition of the invention, for use in the prevention or treatment of an HBV infection or of an HBV-induced disease in mammal in need thereof.

In yet a further aspect, provided herein is a product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound of Formula (I) or the pharmaceutical composition according to the invention, as described herein, and wherein said second compound is an HBV inhibitor. Said HBV inhibitor may be chosen from among:
cytokines having HBV replication inhibition activity,
antibodies having immune checkpoint modulation activity,
substituted pyrimidines having HBV capsid assembly inhibition activity or having TLR agonist activity,
antiretroviral nucleoside analogues, and
the combinations thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In an embodiment, any of the methods provided herein can further comprise administering to the individual at least one additional therapeutic agent selected from the group consisting of an HBV polymerase inhibitor, immunomodulatory agents, interferon, viral entry inhibitor, viral maturation inhibitor, capsid assembly modulator, reverse transcriptase inhibitor, a cyclophilin/TNF inhibitor, a TLR-agonist, an HBV vaccine, and any combination thereof.

In a still further aspect, a process is provided for producing the compound of Formula (I) as described herein, the process comprising:

reacting a compound of Formula (I-2)

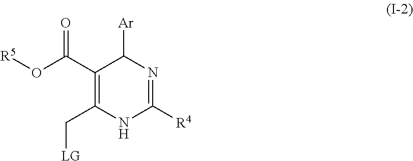

wherein Ar, R⁴-R⁵ are as defined in Formula (I), and LG represents a suitable leaving group, such as for example, bromo; with a compound of Formula (V)

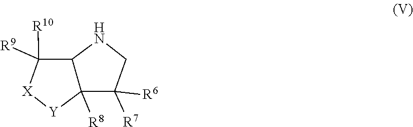

wherein R⁶-R¹⁰, X and Y are as defined in Formula (I);

under suitable nucleophilic substitution conditions, for example, in the presence of a suitable base, such as for example triethanolamine.

### DESCRIPTION

Provided herein are compounds, e.g., the compounds of Formula (I), or pharmaceutically acceptable salts thereof as described herein, that may be useful in the treatment and prevention of HBV infection in a subject.

Without being bound to any particular mechanism of action, these compounds are believed to modulate or disrupt HBV assembly and other HBV core protein functions necessary for HBV replication or the generation of infectious particles and/or may disrupt HBV capsid assembly leading to empty capsids with greatly reduced infectivity or replication capacity. In other words, the compounds provided herein may act as capsid assembly modulators.

There is still a need for compounds with HBV antiviral activity with an advantageous balance of properties, for example potent antiviral activity, favorable metabolic properties, tissue distribution, safety and pharmaceutical profiles, and are suitable for use in humans. It is accordingly an object of the present invention to provide compounds that overcome at least some of these problems. The disclosed compounds may modulate (e.g., accelerate, delay, inhibit, disrupt or reduce) normal viral capsid assembly or disassembly, bind capsid or alter metabolism of cellular polyproteins and precursors. The modulation may occur when the capsid protein is mature, or during viral infectivity. Disclosed compounds can be used in methods of modulating the activity or properties of HBV cccDNA, or the generation or release of HBV RNA particles from within an infected cell.

In one embodiment, the compounds described herein may be suitable for monotherapy and may be effective against natural or native HBV strains and against HBV strains resistant to currently known drugs. In another embodiment, the compounds described herein may be suitable for use in combination therapy.

### Definitions

Listed below are definitions of various terms used to describe this invention. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of 20% or 10%, including ±5%, ±1%, and ±0.1% from the specified value, as such variations are appropriate to perform the disclosed methods.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In one embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity or is lethal to the virus.

As used herein, the term “treatment” or “treating” is defined as the application or administration of a therapeutic agent, i.e., a disclosed compound (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection, or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445 and Journal of Pharmaceutical Science, 66, 1-19 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention.

Other additional ingredients that may be included in the pharmaceutical compositions used in the practice of the invention are known in the art and described, for example in Remington's Pharmaceutical Sciences (Genaro, Ed., Mack Publishing Co., 1990, Easton, Pa.), which is incorporated herein by reference.

As used herein, the term “alkyl,” by itself or as part of another substituent means, unless otherwise stated, a straight or branched chain hydrocarbon having the number of carbon atoms designated (i.e., C₁₋₃alkyl means an alkyl having one to three carbon atoms, C₁₋₄alkyl means an alkyl having one to four carbons and includes straight and branched chains, C₁₋₆alkyl means an alkyl having one to six carbon atoms and includes straight and branched chains, C₁-C₉alkyl means an alkyl having one to nine carbon atoms and includes straight and branched chains). Examples include methyl, ethyl, propyl, isopropyl, butyl, isobutyl, tert-butyl. Embodiments of alkyl include, but are not limited to, C₁₋₉alkyl, C₁₋₆alkyl, C₁₋₄alkyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

The notation “C₃₋₇cycloalkyl” as used herein alone or as part of another group, defines a saturated cyclic hydrocarbon having from 3 to 7 carbon atoms, such as cyclopropyl, cyclobutyl, cyclopentyl, cyclohexyl and cycloheptyl. Particular C₃₋₇cycloalkyl groups are cyclopropyl, cyclobutyl, cyclopentyl and cyclohexyl.

The notation “4- to 8-membered saturated ring in which 1 or 2 of the ring members is a heteroatom each independently selected from the group consisting of N, O, and S” refers to a heteroalicyclic group containing 1 or 2 heteroatoms, each selected from N, O and S. In one embodiment, each heterocyclyl group has from 4 to 8 atoms in its ring system, with the proviso that the ring of said group does not contain two adjacent O or S atoms. The heterocyclic system may be attached to the remainder of the molecule, unless otherwise stated, at any heteroatom or carbon atom that affords a stable structure, as a mono-radical, or at any carbon atom that affords a stable structure, as a di-radical. Particular examples include azetidinyl, pyrrolidinyl, piperidinyl, oxetanyl, tetrahydrofuranyl, and tetrahydropyranyl; more in particular azetidinyl, pyrrolidinyl, and piperidinyl, each of which may be optionally substituted with one or more substituents, each independently selected from C₁₋₄alkyl and OH.

The notation “5- to 6-membered aromatic ring in which 1, 2, 3 or 4 of the ring members is a heteroatom each independently selected from N, O, or S” refers to a heterocycle having aromatic character. Particular examples include thiazolyl, oxazolyl, pyrazolyl, thiadiazolyl, oxadiazolyl, pyridyl, and pyrimidinyl.

The notation “5- to 11-membered bicyclic saturated carbocyclyl” includes fused, spiro and bridged saturated carbocycles. Fused bicyclic groups arc two cycles that share two atoms and the bond between these atoms. Spiro bicyclic groups arc two cycles that arc joined at a single atom. Bridged bicyclic groups are two cycles that share more than two atoms. Particular examples include:

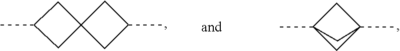

where “- - -” represents the bond of attachment to the remainder of the molecule of Formula (I).

Whenever the term “substituted” is used in the present invention, it is meant, unless otherwise is indicated or is clear from the context, to indicate that one or more hydrogens, in particular from 1 to 3 hydrogens, preferably 1 or 2 hydrogens, more preferably 1 hydrogen, on the atom or radical indicated in the expression using “substituted” are replaced with a selection from the indicated group, provided that the normal valency is not exceeded, and that the substitution results in a chemically stable compound, i.e. a compound that is sufficiently robust to survive isolation to a useful degree of purity from a reaction mixture, and formulation into a therapeutic agent.

When two or more substituents are present on a moiety they may, unless otherwise is indicated or is clear from the context, replace hydrogens on the same atom or they may replace hydrogen atoms on different atoms in the moiety.

As used herein, the terminology “selected from . . . ” (e.g., “R¹ is selected from A, B and C”) is understood to be equivalent to the terminology “selected from the group consisting of . . . ” (e.g., “R¹ is selected from the group consisting of A, B and C”).

In one embodiment, the invention relates to a compound of Formula (II),

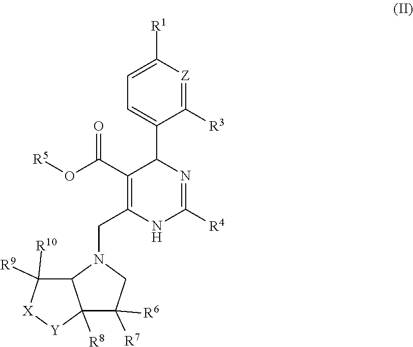

wherein

Z is N or CR²;

R¹, R² and R³ are each independently selected from the group consisting of H, halo, OH, and C₁₋₃alkyl;

the other variable groups are as defined in Formula (I).

In one embodiment, the invention relates to a compound of Formulae (II), as defined hereinbefore, wherein:

Z is CR²;

R¹, R² and R³ are each independently selected from the group consisting of H, halo, and C₁₋₃alkyl;

R⁴ is selected from the group consisting of thiazolyl, imidazolyl, and oxazolyl, each of which may be optionally substituted with one or more methyl substituents;

R⁵ is C₁₋₄alkyl;

R⁶, R⁷ and R⁸ are each independently selected from the group consisting of H and halo;

R⁹ and R¹⁰ are each independently selected from the group consisting of H and halo; or

R⁹ and R¹⁰, together with the carbon atom to which they are attached, form C(═O);

X is selected from the group consisting of CH₂, C(═O), O, S, S(═O), S(═O)₂, NH, NR^{11a}, and CHR^{12a}; and

Y is selected from the group consisting of CH₂, C(═O), O, NH, NR^{11b}, and CHR^{12b};

wherein

R^{11a}, R^{11b}, R^{12a}, and R^{12b} are each independently selected from the group consisting of —CN; —C₁₋₆alkyl, —COOR^{x}; —C₁₋₆alkyl-C(═O)—NR—S(═O)₂—C₁₋₆alkyl; —C₁₋₉alkyl-COOR^{x}, in particular —C₁₋₆alkyl-COOR^{x}; —C₁₋₆alkyl-O—C₁₋₆alkyl-COOR^{x}; -Cy-COOR^{x}; —C₁₋₆alkyl-Cy-COOR^{x}; —C₁₋₆alkyl-Cy-C₁₋₆alkyl-COOR^{x}; —C(═O)—C₁₋₆alkyl; —C(═O)—C₁₋₆alkyl-COOR^{x}; —C(═O)—Cy-COOR^{x}; —C(═O)—O—C₁₋₆alkyl-COOR^{x}; —C(═O)—C₁₋₆alkyl-O—C₁₋₆alkyl-COOR^{x}; —C(═O)H; —C(═O)—NR^{a}R^{b}; —C(═O)-Het¹; —C(═O)—Cy; —C(═O)—NR^{c}—C₁₋₆alkyl-COOR^{x}; —C(═O)—C₁₋₆alkyl-NR^{c}—C₁₋₆alkyl-COOR^{x}; —C(═O)—NR^{c}—CO—NR^{a}R^{b}; —C(═O)—NR^{c}-Cy-COOR^{x}; —C(═O)—NR^{c}—S(═O)₂—C₁₋₆alkyl; —C(═O)—C(═O)-Het¹; —C(═O)—C(═O)—O—C₂₋₆alkenyl; -Het¹-C₁₋₆alkyl-COOR^{x}; —C₁₋₆alkyl-C(═O)-Het¹-COOR^{x}; -Het²-COOR^{x}; —C₁₋₆alkyl-Het²; —C₁₋₆alkyl-Het²-COOR^{x}; -Het²-C₁₋₆alkyl-COOR^{x}; —C₁₋₆alkyl-Het²-C₁₋₆alkyl-COOR^{x}; —NR^{c}—C₁₋₆alkyl-COOR^{x}; —O—C₁₋₉alkyl-COOR^{x}, in particular —O—C₁₋₆alkyl-COOR^{x}; —S(═O)₂—NR^{a}R^{b}; —S(═O)₂—C₁₋₆alkyl; —S(═O)₂—C₁₋₆alkyl-COOR^{x}; —S(═O)₂—Cy-COOR^{x}; —S(═O)₂—NR^{c}-Cy-COOR^{x}; —S(═O)₂—NR^{c}-Het²; —S(═O)₂-Het¹-COOR^{x}; —S(═O)₂—NR^{c}—C(═O)—C₁₋₆alkyl; —C(═O)—NR^{c}—S(═O)₂—C₁₋₆alkyl; and —C₁₋₆ alkyl-C(═O)—NR^{c}—S(═O)₂—C₁₋₆alkyl.

In an additional embodiment, the invention relates to a compound of Formulae (I) or (II), as defined hereinbefore, wherein:

R^{11a}, R^{11b}, R^{12a}, and R^{12b} are each independently selected from the group consisting of —CN; —C₁₋₆alkyl; —COOH; —C₁₋₉alkyl-COOH; —C₁₋₆alkyl-O—C₁₋₆alkyl-COOH; -Cy-COOH; —C₁₋₆ alkyl-Cy-COOH; -Cy-C₁₋₆alkyl-COOH; —C₁₋₆alkyl-Cy-C₁₋₆alkyl-COOH; —C(═O)—C₁₋₆alkyl; —C(═O)—C₁₋₆alkyl-COOH; —C(═O)—Cy-COOH; —C(═O)—O—C₁₋₆alkyl-COOH; —C(═O)—C₁₋₆alkyl-O—C₁₋₆alkyl-COOH; —C(═O)—NR^{a}R^{b}; —C(═O)-Het¹; —C(═O)—NR^{c}—C₁₋₆alkyl-COOH; —C(═O)—C₁₋₆ alkyl-NR^{c}—C₁₋₆alkyl-COOH; —C(═O)—NR^{c}—COOH; —C(═O)—NR^{c}—CO—NR^{a}R^{b}; —C(═O)—NR^{c}-Cy-COOH; —C(═O)-Het¹-COOH; —C(═O)—NR^{c}-Het¹-COOH; —C(═O)—C(═O)—NR^{a}R^{b}; —C(═O)—C(═O)-Het¹; —C(═O)—C(═O)—O—C₂₋₆alkenyl; -Het¹-COOH; -Het¹-C₁₋₆alkyl-COOH; —C₁₋₆alkyl-Het¹-COOH; —C₁₋₆alkyl-Het¹-C₁₋₆alkyl-COOH; —C₁₋₆alkyl-C(═O)-Het¹-COOH; -Het²-COOH; —C₁₋₆alkyl-Het²-COOH; -Het²-C₁₋₆alkyl-COOH; —C₁₋₆alkyl-Het²-C₁₋₆alkyl-COOH; —NR^{c}—C₁₋₆alkyl-COOH; —NR^{c}—Cy-COOH; —NR^{c}-Het¹-COOH; —O—C₁₋₉alkyl-COOH; —S(═O)₂—NR^{a}R^{b}; —S(═O)₂—C₁₋₆alkyl; —S(═O)₂—C₁₋₆alkyl-COOH; —S(═O)₂—Cy-COOH; —S(═O)₂—Cy-C₁₋₆alkyl-COOH; —S(═O)₂—NR^{c}-Cy-COOH; —S(═O)₂—NR^{c}-Het²; —S(═O)₂-Het¹-COOH; —S(═O)₂-Het¹-C₁₋₆alkyl-COOH; —S(═O)₂—NR^{c}—C(═O)—C₁₋₆alkyl; —C(═O)—NR^{c}—S(═O)₂—C₁₋₆alkyl; and —C₁₋₆alkyl-C(═O)—NR^{c}—S(═O)₂—C₁₋₆alkyl; and the rest of variables are as defined herein.

In a further embodiment, the invention relates to a compound of Formulae (I) or (II), as defined hereinbefore, wherein:

R^{11a}, R^{11b}, R^{12a}, and R^{12b} are each independently selected from the group consisting of —CN; —C₁₋₆alkyl; —COOH; —C₁₋₉alkyl-COOH, in particular —C₁₋₆alkyl-COOH; —C₁₋₆alkyl-O—C₁₋₆alkyl-COOH; -Cy-COOH; —C₁₋₆alkyl-Cy-COOH; —C₁₋₆alkyl-Cy-C₁₋₆alkyl-COOH; —C(═O)—C₁₋₆alkyl; —C(═O)—C₁₋₆alkyl-COOH; —C(═O)—Cy-COOH; —C(═O)—O—C₁₋₆alkyl-COOH; —C(═O)—C₁₋₆alkyl-O—C₁₋₆alkyl-COOH; —C(═O)—NR^{a}R^{b}; —C(═O)-Het¹; —C(═O)—NR^{c}—C₁₋₆alkyl-COOH; —C(═O)—C₁₋₆alkyl-NR^{c}—C₁₋₆alkyl-COOH; —C(═O)—NR^{c}—CO—NR^{a}R^{b}; —C(═O)—NR^{c}-Cy-COOH; —C(═O)—C(═O)-Het¹; —C(═O)—C(═O)—O—C₂₋₆alkenyl; -Het¹-C₁₋₆alkyl-COOH; —C₁₋₆alkyl-C(═O)-Het¹-COOH; -Het²-COOH; —C₁₋₆alkyl-Het²-COOH; -Het²-C₁₋₆alkyl-COOH; —C₁₋₆alkyl-Het²-C₁₋₆alkyl-COOH; —NR^{c}—C₁₋₆alkyl-COOH; —O—C₁₋₉alkyl-COOH, in particular —O—C₁₋₆alkyl-COOH; —S(═O)₂—NR^{a}R^{b}; —S(═O)₂—C₁₋₆alkyl; —S(═O)₂—C₁₋₆alkyl-COOH; —S(═O)₂-Cy-COOH; —S(═O)₂—NR^{c}-Cy-COOH; —S(═O)₂—NR^{c}-Het²; —S(═O)₂-Het¹-COOH; —S(═O)₂—NR^{c}—C(═O)—C₁₋₆alkyl; —C(═O)—NR^{c}—S(═O)₂—C₁₋₆alkyl; and —C₁₋₆alkyl-C(═O)—NR^{c}—S(═O)₂—C₁₋₆alkyl; and the rest of variables are as defined herein.

In a further embodiment, the invention relates to a compound of Formulae (I) or (II), as defined hereinbefore, wherein:

R^{1a}, R^{11b}, R^{12a}, and R^{12b} are each independently selected from the group consisting of —CN; —C₁₋₆alkyl; —COOH; —C₁₋₉alkyl-COOH, in particular —C₁₋₆alkyl-COOH; -Cy-COOH; —C₁₋₆ alkyl-Cy-COOH; —C₁₋₆alkyl-Cy-C₁₋₆alkyl-COOH; —C(═O)—C₁₋₆alkyl; —C(═O)—C₁₋₆alkyl-COOH; —C(═O)—Cy-COOH; —C(═O)—O—C₁₋₆alkyl-COOH; —C(═O)—C₁₋₆alkyl-O—C₁₋₆alkyl-COOH; —C(═O)—NR^{a}R^{b}; —C(═O)-Het¹; —C(═O)—NR—C₁₋₆alkyl-COOH; —C(═O)—C₁₋₆alkyl-NR^{c}—C₁₋₆alkyl-COOH; —C(═O)—NR^{c}—CO—NR^{a}R^{b}; —C(═O)—NR^{c}-Cy-COOH; —C(═O)—C(═O)-Het¹; —C(═O)—C(═O)—O—C₂₋₆alkenyl; -Het¹-C₁₋₆alkyl-COOH; —C₁₋₆alkyl-C(═O)-Het¹-COOH; -Het²-COOH; —S(═O)₂—NR^{a}R^{b}; —S(═O)₂—C₁₋₆alkyl; —S(═O)₂—C₁₋₆alkyl-COOH; —S(═O)₂—NR^{c}—C(═O)—C₁₋₆alkyl; —C(═O)—NR^{c}—S(═O)₂—C₁₋₆alkyl; and —C₁₋₆alkyl-C(═O)—NR^{c}—S(═O)₂—C₁₋₆alkyl.

In a further embodiment, the invention relates to a compound of Formulae (I) or (II), as defined hereinbefore, wherein:

R^{11a}, R^{11b}, R^{12a}, and R^{12b} are each independently selected from the group consisting of —COOH; —C₁₋₆alkyl; —C₁₋₆alkyl —C₁₋₆alkyl-COOH, -Cy-COOH, —C(═O)—C₁₋₆alkyl-COOH, —C(═O)—NR^{a}R^{b}, and —S(═O)₂—NR^{c}—C(═O)—C₁₋₆alkyl.

In a particular embodiment, R¹, R² and R³ are each independently selected from the group consisting of H, halo, OH, and methyl; and the rest of variables are as defined herein. In a further embodiment, R¹ is hydrogen or fluoro; R² is hydrogen, fluoro or hydroxy; R³ is selected from chloro and methyl; and the rest of variables are as defined herein.

In a particular embodiment, R⁴ is selected from the group consisting of thiazolyl, imidazolyl, oxazolyl and pyridyl, each of which may be optionally substituted with one methyl substituent; and the rest of variables are as defined herein. In an additional embodiment, R⁴ is selected from the group consisting of thiazolyl, imidazolyl, oxazolyl, each of which may be optionally substituted with one methyl substituent; and the rest of variables are as defined herein. In an additional embodiment, R⁴ is selected from the group consisting of thiazol-2-yl, 1-methyl-imidazol-2-yl and 5-methyl-oxazol-4-yl; more in particular, thiazol-2-yl and 5-methyl-oxazol-4-yl; and the rest of variables are as defined herein.

In a further embodiment, R⁵ is methyl or ethyl; and the rest of variables are as defined herein.

In a further embodiment, R⁶, R⁷ and R⁸ are each independently selected from hydrogen and halo; more in particular, from hydrogen and fluoro; and the rest of the variables are as defined herein. In a further embodiment, R⁶ and R⁷ are each hydrogen and R⁸ is fluoro; or R⁶ and R⁷ are each fluoro and R⁸ is hydrogen; and the rest of the variables are as defined herein. In a further embodiment, R⁶ and R⁸ are each hydrogen and R⁷ is fluoro; and the rest of the variables are as defined herein.

In an additional embodiment, R⁹ and R¹⁰ are each independently selected from hydrogen and halo; or R⁹ and R¹⁰, together with the carbon atom to which they are attached, form C(═O); and the rest of the variables are as defined herein. In an additional embodiment, R⁹ and R¹⁰ are each independently selected from hydrogen and fluoro; or R⁹ and R¹⁰, together with the carbon atom to which they are attached, form C(═O); and the rest of the variables are as defined herein.

In an additional embodiment, R^{a}, R^{b} and R^{c} are each independently selected from H and methyl; more in particular, H; and the rest of the variables are as defined herein.

In an embodiment R^{x} is selected from H and —C₁₋₆alkyl; in particular, H and —C₁₋₄alkyl; and the rest of the variables are as defined herein. In a further embodiment, R^{x} is H, and the rest of the variables are as defined herein.

In a yet further embodiment, Cy is selected from the group consisting of cyclopropyl, cyclobutyl, and cyclohexyl; and the rest of the variables are as defined herein.

In a further embodiment, Het¹ is selected from the group consisting of azetidinyl, pyrrolidinyl, and piperidinyl, each of which may be optionally substituted with one or more substituents, each independently selected from methyl and OH; and the rest of the variables are as defined herein.

In another embodiment, Het² is selected from the group consisting of pyrazolyl, thiazolyl, pyrimidinyl and thiadiazolyl, each of which may be optionally substituted with one or more methyl substituents; and the rest of the variables are as defined herein.

In a further embodiment,

X is selected from the group consisting of CH₂, O, NR^{11a}, and CHR^{12a};

Y is selected from the group consisting of CH₂, C(═O), NR^{11b}, and CHR^{12b};

R^{11a} is selected from the group consisting of

—C₁₋₉alkyl-COOH; —C₁₋₆alkyl-O—C₁₋₆alkyl-COOH; -Cy-COOH; —C₁₋₆alkyl-C(═O)—NR^{c}—S(═O)₂—C₁₋₆alkyl; —C₁₋₆alkyl-Cy-COOH; -Cy-C₁₋₆alkyl-COOH; —C₁₋₆alkyl-Cy-C₁₋₆alkyl-COOH; —C(═O)—C₁₋₆alkyl; —C(═O)—C₁₋₆alkyl-COOH; —C(═O)—Cy-COOH; —C(═O)—O—C₁₋₆alkyl-COOH; —C(═O)—C₁₋₆alkyl-O—C₁₋₆alkyl-COOH; —C(═O)H; —C(═O)—NR^{a}R^{b}; —C(═O)—Cy; —C(═O)—NR^{c}—C₁₋₆alkyl-COOH; —C(═O)—C₁₋₆alkyl-NR^{c}—C₁₋₆alkyl-COOH; —C(═O)—NR^{c}—COOH; —C(═O)—NR^{c}—Cy-COOH; —C(═O)—NR^{c}—S(═O)₂—C₁₋₆alkyl; —C(═O)-Het¹-COOH; —C(═O)—NR^{c}-Het¹-COOH; —C(═O)—C(═O)—NR^{a}R^{b}; -Het¹-COOH; -Het¹-C₁₋₆alkyl-COOH; —C₁₋₆alkyl-Het¹-COOH; —C₁₋₆ alkyl-Het¹-C₁₋₆alkyl-COOH; -Het²-COOH; —C₁₋₆alkyl-Het²; —C₁₋₆alkyl-Het²-COOH; -Het²-C₁₋₆alkyl-COOH; —C₁₋₆alkyl-Het²-C₁₋₆alkyl-COOH; —S(═O)₂—C₁₋₆alkyl-COOH; —S(═O)₂—Cy-COOH; —S(═O)₂—Cy-C₁₋₆alkyl-COOH; —S(═O)₂-Het¹-COOH; —S(═O)₂-Het¹-C₁₋₆alkyl-COOH; —S(═O)₂—NR^{c}—C(═O)—C₁₋₆alkyl; —C(═O)—NR^{c}—S(═O)₂—C₁₋₆alkyl; and —C₁₋₆alkyl-C(═O)—NR^{c}—S(═O)₂—C₁₋₆alkyl;

R^{12a} is selected from the group consisting of —C₁₋₆alkyl, and —COOH;

R^{11b} and R^{12b} are independently selected from the group consisting of

—C₁₋₉alkyl-COOH; —C₁₋₆alkyl-O—C₁₋₆alkyl-COOH; -Cy-COOH; —C₁₋₆alkyl-C(═O)—NR^{c}—S(═O)₂—C₁₋₆alkyl; —C₁₋₆alkyl-Cy-COOH; -Cy-C₁₋₆alkyl-COOH; —C₁₋₆alkyl-Cy-C₁₋₆alkyl-COOH; —C(═O)—C₁₋₆alkyl; —C(═O)—C₁₋₆alkyl-COOH; —C(═O)—Cy-COOH; —C(═O)—O—C₁₋₆alkyl-COOH; —C(═O)—C₁₋₆alkyl-O—C₁₋₆alkyl-COOH; —C(═O)—NR^{a}R^{b}; —C(═O)—Cy; —C(═O)—NR^{c}—C₁₋₆alkyl-COOH; —C(═O)—C₁₋₆alkyl-NR^{c}—C₁₋₆alkyl-COOH; —C(═O)—NR^{c}—COOH; —C(═O)—NR^{c}—CO—NR^{a}R^{b}; —C(═O)—NR^{c}-Cy-COOH; —C(═O)—NR^{c}—S(═O)₂—C₁₋₆alkyl; —C(═O)-Het¹-COOH; —C(═O)—NR^{c}-Het¹-COOH; -Het¹-COOH; -Het¹-C₁₋₆alkyl-COOH; —C₁₋₆alkyl-Het¹-COOH; —C₁₋₆alkyl-Het¹-C₁₋₆alkyl-COOH; —C₁₋₆alkyl-C(═O)-Het¹-COOH; -Het²-COOH; —C₁₋₆alkyl-Het²; —C₁₋₆alkyl-Het²-COOH; -Het²-C₁₋₆alkyl-COOH; —C₁₋₆alkyl-Het²-C₁₋₆alkyl-COOH; —O—C₁₋₉alkyl-COOH; —S(═O)₂—NR^{a}R^{b}; —S(═O)₂—C₁₋₆alkyl; —S(═O)₂—C₁₋₆alkyl-COOH; —S(═O)₂—Cy-COOH; —S(═O)₂—Cy-C₁₋₆alkyl-COOH; —S(═O)₂—NR^{c}-Cy-COOH; —S(═O)₂—NR^{c}-Het²; —S(═O)₂-Het¹-COOH; —S(═O)₂-Het¹-C₁₋₆alkyl-COOH; —C(═O)—NR^{c}—S(═O)₂—C₁₋₆alkyl; and —C₁₋₆alkyl-C(═O)—NR^{c}—S(═O)₂—C₁₋₆alkyl.

In a particular embodiment, the compound of Formula (I) is selected from the compounds satisfying the following Formulae (I-A) to (I-E):

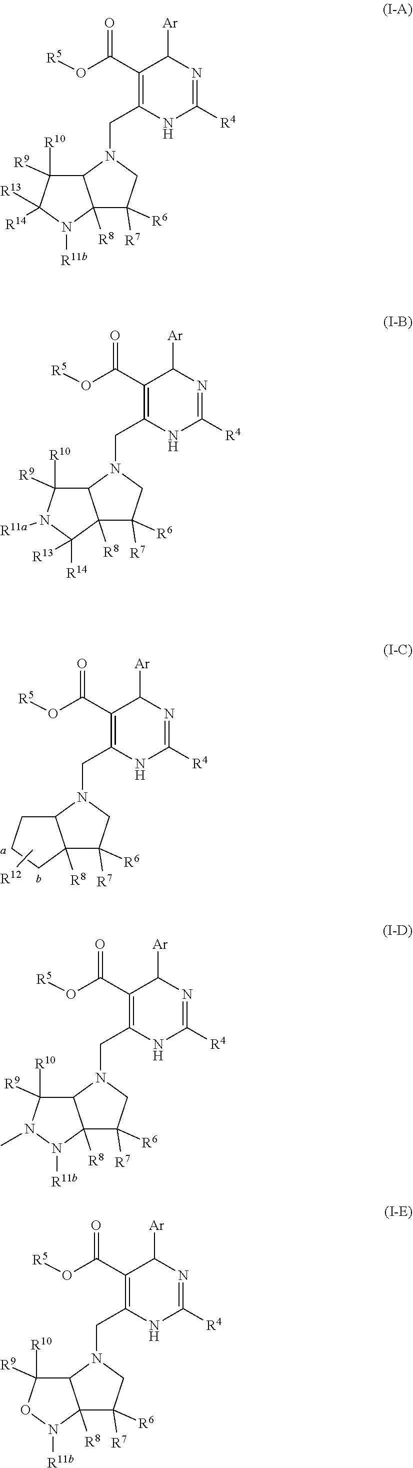

wherein, R¹³ and R¹⁴ are both hydrogen; or R¹³ and R¹⁴, together with the carbon atom to which they are attached, form C(═O); a and b are the position of attachment for R¹² which reprensents R^{12a} and/or R^{12b}; and Ar, R⁴-R¹² are as defined hereinbefore.

In a further embodiment, the invention relates to a compound of Formula (I-A), (I-B), (I-C), (I-D) or (I-E) as defined hereinbefore, wherein:

Ar is selected from the group consisting of phenyl, and pyridyl, optionally substituted with one or more substituents selected from the group consisting of C₁₋₄alkyl, hydroxyl, halogen, and CN;

R⁴ is selected from the group consisting of thiazolyl, imidazolyl, and oxazolyl, each of which may be optionally substituted with one or more methyl substituents;

R⁵ is C₁₋₄alkyl; in particular, methyl or ethyl;

R⁶, R⁷ and R⁸ are each independently selected from the group consisting of H and halo; in particular, H and fluoro;

R⁹ and R¹⁰ are each independently selected from the group consisting of H and halo, in particular hydrogen and fluoro; or R⁹ and R¹⁰, together with the carbon atom to which they are attached, form C(═O);

R^{11a}, R^{11b}, R^{12a}, and R^{12b} are each independently selected from the group consisting of —COOH; —C₁₋₆alkyl; —C₁₋₆alkyl-COOH, -Cy-COOH, —C(═O)—C₁₋₆alkyl-COOH, —C(═O)—NR^{a}R^{b}, and —S(═O)₂—NR^{c}—C(═O)—C₁₋₆alkyl.

In a particular embodiment, the compound of Formula (I) is selected from the compounds satisfying the following Formulae (II-A) to (II-E):

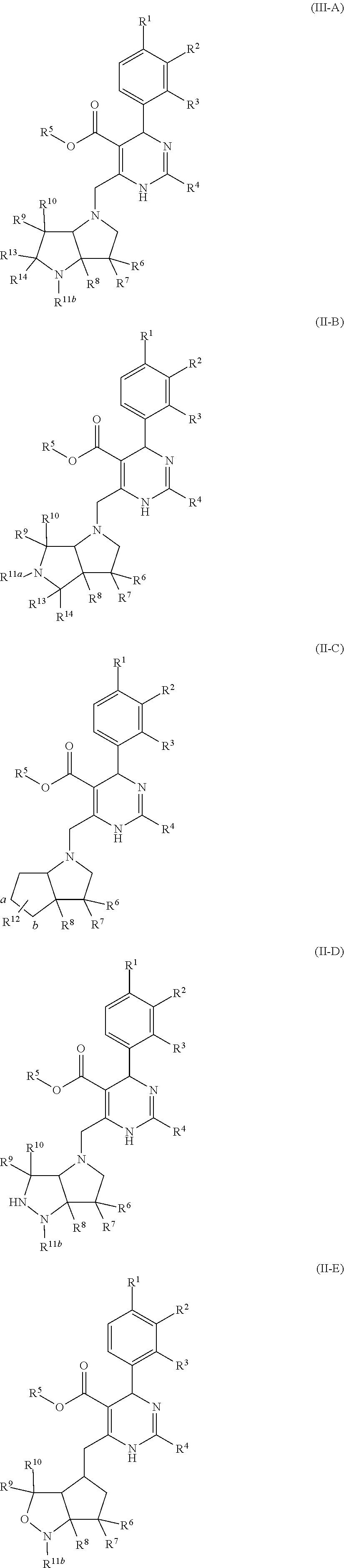

wherein, R¹³ and R¹⁴ are both hydrogen; or R¹³ and R¹⁴, together with the carbon atom to which they are attached, form C(═O); a and b are the position of attachment for R¹² which represents R^{12a} and/or R^{12b}; and R¹-R¹² are as defined hereinbefore.

In a further embodiment, the invention relates to a compound of Formula (II-A), (II-B), (II-C), (II-D), or (II-E) as defined hereinbefore, wherein:

R¹, R² and R³ are each independently selected from the group consisting of H, halo, and C₁₋₃ alkyl; in particular, H, fluoro, chloro, and methyl;

R⁴ is selected from the group consisting of thiazolyl, imidazolyl, and oxazolyl, each of which may be optionally substituted with one or more methyl substituents;

R⁵ is C₁₋₄alkyl; in particular, methyl or ethyl;

R⁶, R⁷ and R⁸ are each independently selected from the group consisting of H and halo; in particular, H and fluoro;

R⁹ and R¹⁰ are each independently selected from the group consisting of H and halo, in particular hydrogen and fluoro; or R⁹ and R¹⁰, together with the carbon atom to which they are attached, form C(═O);

R^{11a}, R^{11b}, R^{12a}, and R^{12b} are each independently selected from the group consisting of —COOH; —C₁₋₆alkyl; —C₁₋₆alkyl-COOH, -Cy-COOH, —C(═O)—C₁₋₆alkyl-COOH, —C(═O)—NR^{a}R^{b}, and —S(═O)₂—NR^{c}—C(═O)—C₁₋₆alkyl.

All combinations of the foregoing embodiments are expressly included.

An embodiment relates to a compound is selected from the group consisting of compound satisfying the following formulae

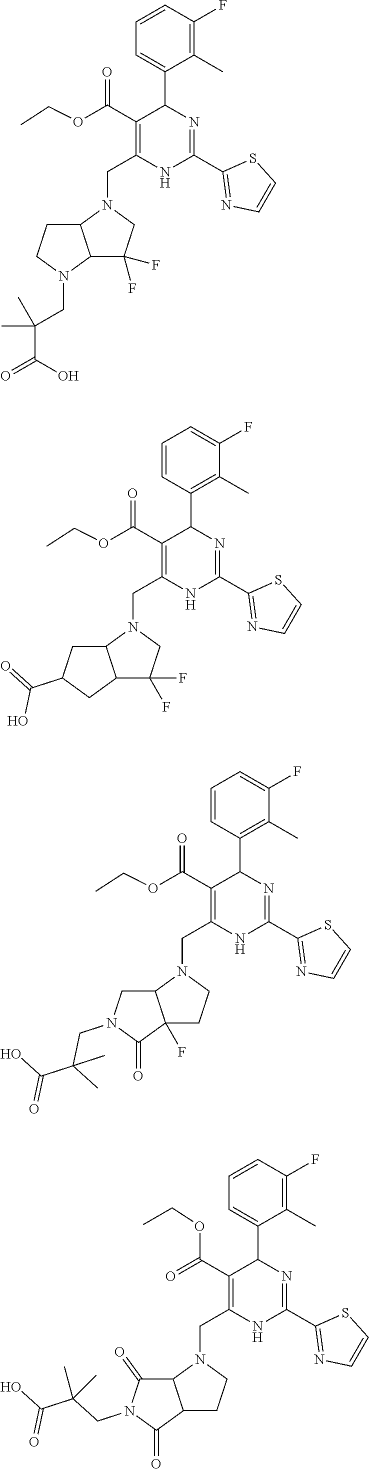 [SMILES: CCOC(=O)C1=C(CN2CC(F)(F)C3C2CCN3CC(C)(C)C(=O)O)NC(c2nccs2)=NC1c1cccc(F)c1C ; CCOC(=O)C1=C(CN2CC(F)(F)C3CC(C(=O)O)CC32)NC(c2nccs2)=NC1c1cccc(F)c1C ; CCOC(=O)C1=C(CN2CCC3(F)C(=O)N(CC(C)(C)C(=O)O)CC23)NC(c2nccs2)=NC1c1cccc(F)c1C ; CCOC(=O)C1=C(CN2CCC3C(=O)N(CC(C)(C)C(=O)O)C(=O)C32)NC(c2nccs2)=NC1c1cccc(F)c1C]

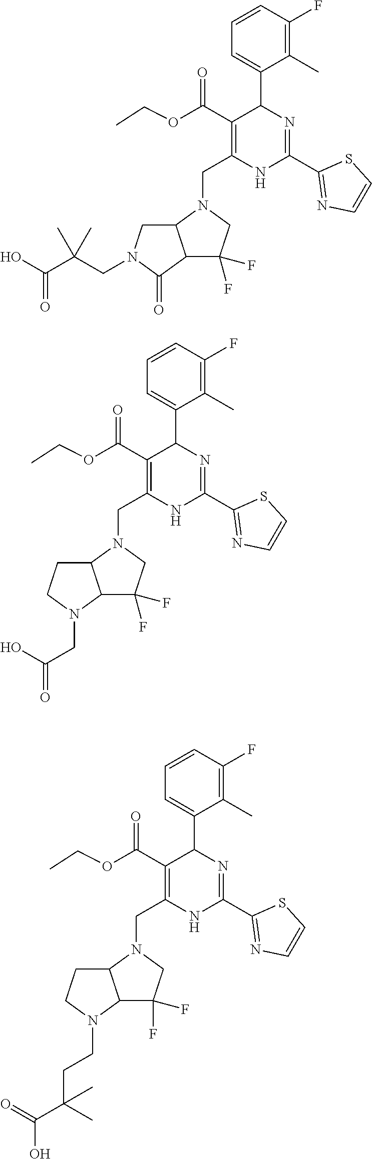

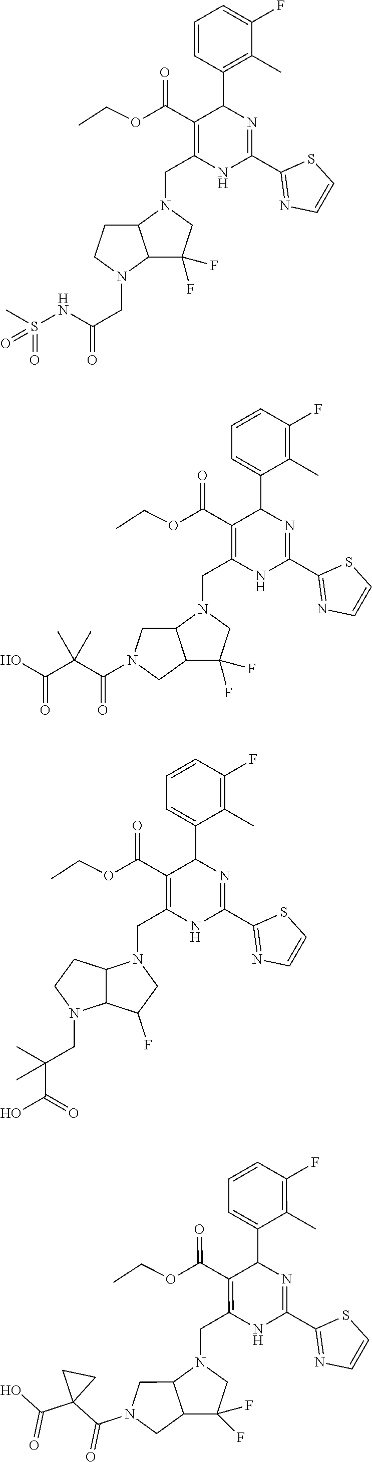 [SMILES: CCOC(=O)C1=C(CN2CC(F)(F)C3C2CCN3CC(=O)NS(C)(=O)=O)NC(c2nccs2)=NC1c1cccc(F)c1C ; CCOC(=O)C1=C(CN2CC(F)(F)C3CN(C(=O)C(C)(C)C(=O)O)CC32)NC(c2nccs2)=NC1c1cccc(F)c1C ; CCOC(=O)C1=C(CN2CC(F)C3C2CCN3CC(C)(C)C(=O)O)NC(c2nccs2)=NC1c1cccc(F)c1C ; CCOC(=O)C1=C(CN2CC(F)(F)C3CN(C(=O)C4(C(=O)O)CC4)CC32)NC(c2nccs2)=NC1c1cccc(F)c1C]

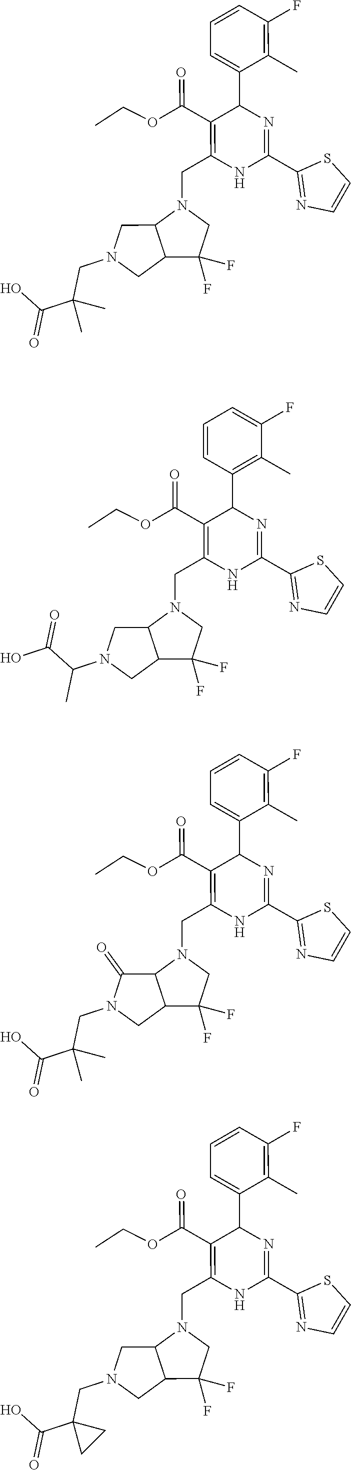 [SMILES: CCOC(=O)C1=C(CN2CC(F)(F)C3CN(CC(C)(C)C(=O)O)CC32)NC(c2nccs2)=NC1c1cccc(F)c1C ; CCOC(=O)C1=C(CN2CC(F)(F)C3CN(C(C)C(=O)O)CC32)NC(c2nccs2)=NC1c1cccc(F)c1C ; CCOC(=O)C1=C(CN2CC(F)(F)C3CN(CC(C)(C)C(=O)O)C(=O)C32)NC(c2nccs2)=NC1c1cccc(F)c1C ; CCOC(=O)C1=C(CN2CC(F)(F)C3CN(CC4(C(=O)O)CC4)CC32)NC(c2nccs2)=NC1c1cccc(F)c1C]

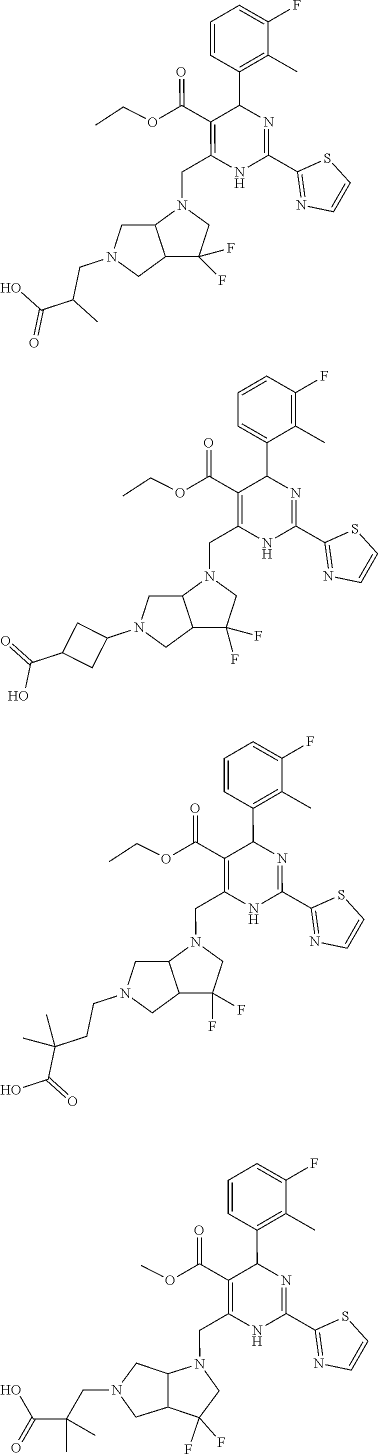 [SMILES: CCOC(=O)C1=C(CN2CC(F)(F)C3CN(CC(C)C(=O)O)CC32)NC(c2nccs2)=NC1c1cccc(F)c1C ; CCOC(=O)C1=C(CN2CC(F)(F)C3CN(C4CC(C(=O)O)C4)CC32)NC(c2nccs2)=NC1c1cccc(F)c1C ; CCOC(=O)C1=C(CN2CC(F)(F)C3CN(CCC(C)(C)C(=O)O)CC32)NC(c2nccs2)=NC1c1cccc(F)c1C ; COC(=O)C1=C(CN2CC(F)(F)C3CN(CC(C)(C)C(=O)O)CC32)NC(c2nccs2)=NC1c1cccc(F)c1C]

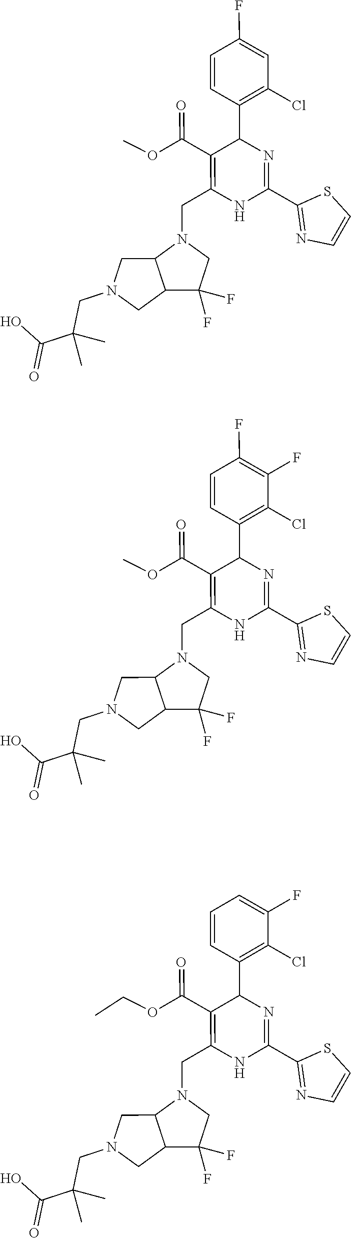 [SMILES: COC(=O)C1=C(CN2CC(F)(F)C3CN(CC(C)(C)C(=O)O)CC32)NC(c2nccs2)=NC1c1ccc(F)cc1Cl ; COC(=O)C1=C(CN2CC(F)(F)C3CN(CC(C)(C)C(=O)O)CC32)NC(c2nccs2)=NC1c1ccc(F)c(F)c1Cl ; CCOC(=O)C1=C(CN2CC(F)(F)C3CN(CC(C)(C)C(=O)O)CC32)NC(c2nccs2)=NC1c1cccc(F)c1Cl]

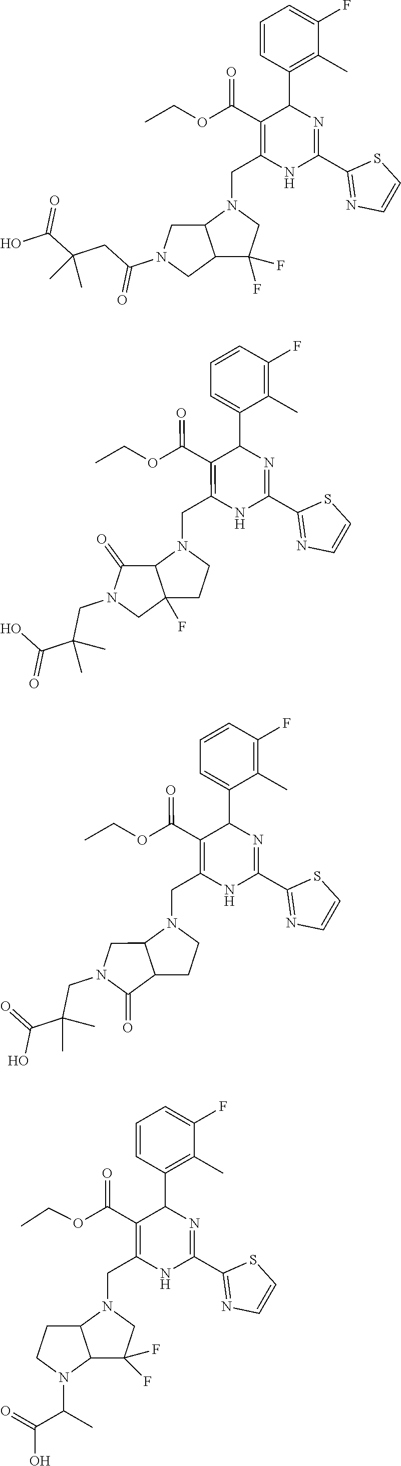 [SMILES: CCOC(=O)C1=C(CN2CC(F)(F)C3CN(C(=O)CC(C)(C)C(=O)O)CC32)NC(c2nccs2)=NC1c1cccc(F)c1C ; CCOC(=O)C1=C(CN2CCC3(F)CN(CC(C)(C)C(=O)O)C(=O)C23)NC(c2nccs2)=NC1c1cccc(F)c1C ; CCOC(=O)C1=C(CN2CCC3C(=O)N(CC(C)(C)C(=O)O)CC32)NC(c2nccs2)=NC1c1cccc(F)c1C ; CCOC(=O)C1=C(CN2CC(F)(F)C3C2CCN3C(C)C(=O)O)NC(c2nccs2)=NC1c1cccc(F)c1C]

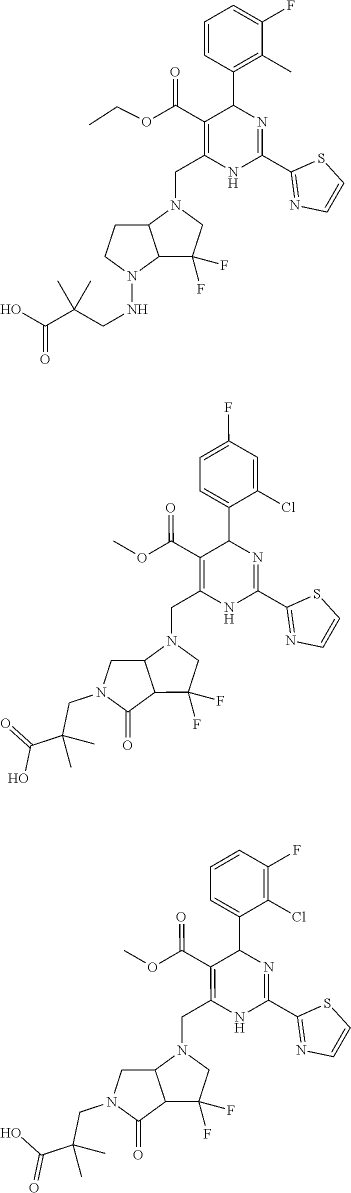 [SMILES: CCOC(=O)C1=C(CN2CC(F)(F)C3C2CCN3NCC(C)(C)C(=O)O)NC(c2nccs2)=NC1c1cccc(F)c1C ; COC(=O)C1=C(CN2CC(F)(F)C3C(=O)N(CC(C)(C)C(=O)O)CC32)NC(c2nccs2)=NC1c1ccc(F)cc1Cl ; COC(=O)C1=C(CN2CC(F)(F)C3C(=O)N(CC(C)(C)C(=O)O)CC32)NC(c2nccs2)=NC1c1cccc(F)c1Cl]

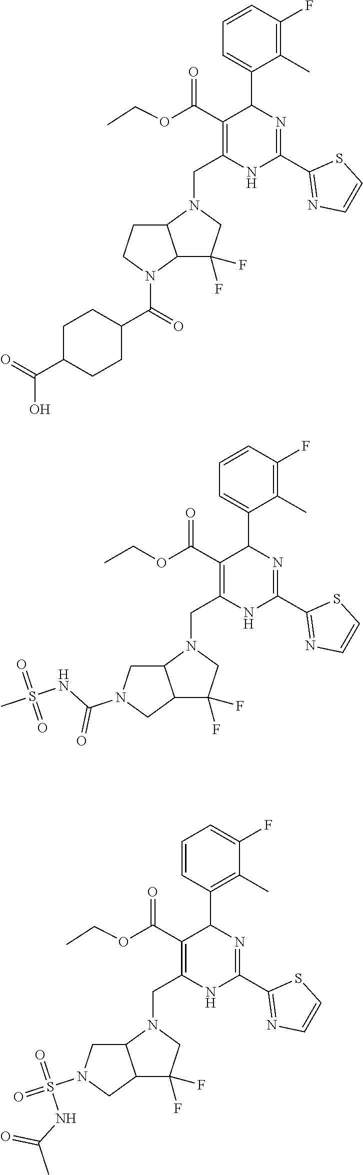 [SMILES: CCOC(=O)C1=C(CN2CC(F)(F)C3C2CCN3C(=O)C2CCC(C(=O)O)CC2)NC(c2nccs2)=NC1c1cccc(F)c1C ; CCOC(=O)C1=C(CN2CC(F)(F)C3CN(C(=O)NS(C)(=O)=O)CC32)NC(c2nccs2)=NC1c1cccc(F)c1C ; CCOC(=O)C1=C(CN2CC(F)(F)C3CN(S(=O)(=O)NC(C)=O)CC32)NC(c2nccs2)=NC1c1cccc(F)c1C]

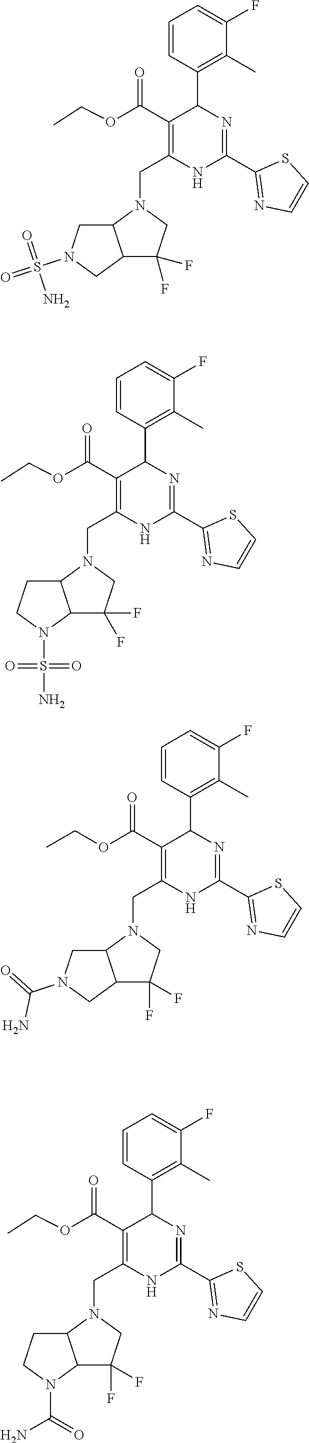 [SMILES: CCOC(=O)C1=C(CN2CC(F)(F)C3CN(S(N)(=O)=O)CC32)NC(c2nccs2)=NC1c1cccc(F)c1C ; CCOC(=O)C1=C(CN2CC(F)(F)C3C2CCN3S(N)(=O)=O)NC(c2nccs2)=NC1c1cccc(F)c1C ; CCOC(=O)C1=C(CN2CC(F)(F)C3CN(C(N)=O)CC32)NC(c2nccs2)=NC1c1cccc(F)c1C ; CCOC(=O)C1=C(CN2CC(F)(F)C3C2CCN3C(N)=O)NC(c2nccs2)=NC1c1cccc(F)c1C]

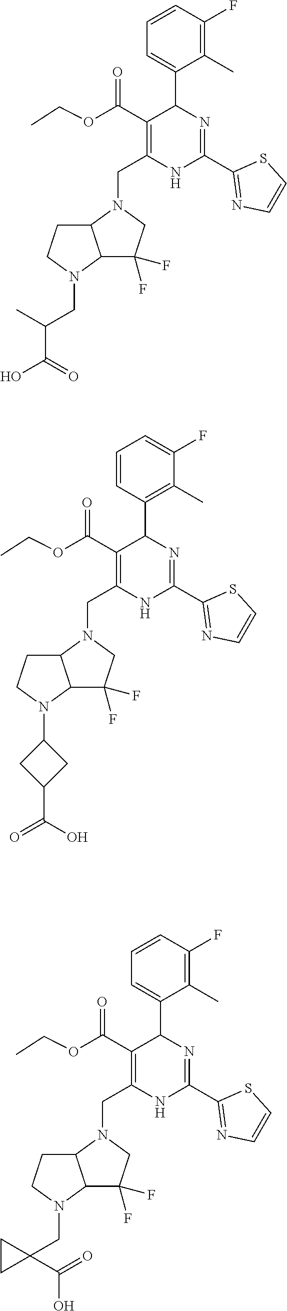 [SMILES: CCOC(=O)C1=C(CN2CC(F)(F)C3C2CCN3CC(C)C(=O)O)NC(c2nccs2)=NC1c1cccc(F)c1C ; CCOC(=O)C1=C(CN2CC(F)(F)C3C2CCN3C2CC(C(=O)O)C2)NC(c2nccs2)=NC1c1cccc(F)c1C ; CCOC(=O)C1=C(CN2CC(F)(F)C3C2CCN3CC2(C(=O)O)CC2)NC(c2nccs2)=NC1c1cccc(F)c1C]

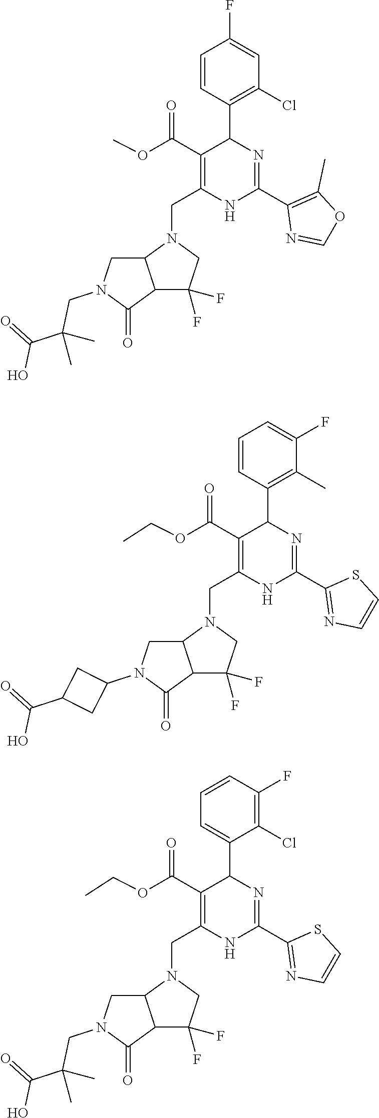 [SMILES: COC(=O)C1=C(CN2CC(F)(F)C3C(=O)N(CC(C)(C)C(=O)O)CC32)NC(c2ncoc2C)=NC1c1ccc(F)cc1Cl ; CCOC(=O)C1=C(CN2CC(F)(F)C3C(=O)N(C4CC(C(=O)O)C4)CC32)NC(c2nccs2)=NC1c1cccc(F)c1C ; CCOC(=O)C1=C(CN2CC(F)(F)C3C(=O)N(CC(C)(C)C(=O)O)CC32)NC(c2nccs2)=NC1c1cccc(F)c1Cl]

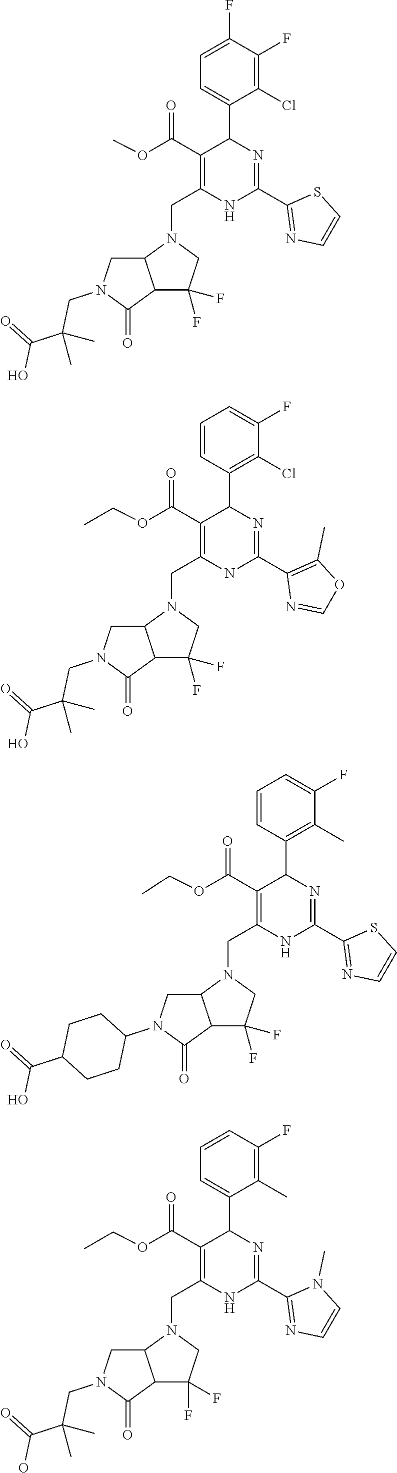 [SMILES: COC(=O)C1=C(CN2CC(F)(F)C3C(=O)N(CC(C)(C)C(=O)O)CC32)NC(c2nccs2)=NC1c1ccc(F)c(F)c1Cl ; CCOC(=O)C1=C(CN2CC(F)(F)C3C(=O)N(CC(C)(C)C(=O)O)CC32)NC(c2ncoc2C)=NC1c1cccc(F)c1Cl ; CCOC(=O)C1=C(CN2CC(F)(F)C3C(=O)N(C4CCC(C(=O)O)CC4)CC32)NC(c2nccs2)=NC1c1cccc(F)c1C ; CCOC(=O)C1=C(CN2CC(F)(F)C3C(=O)N(CC(C)(C)C(=O)O)CC32)NC(c2nccn2C)=NC1c1cccc(F)c1C]

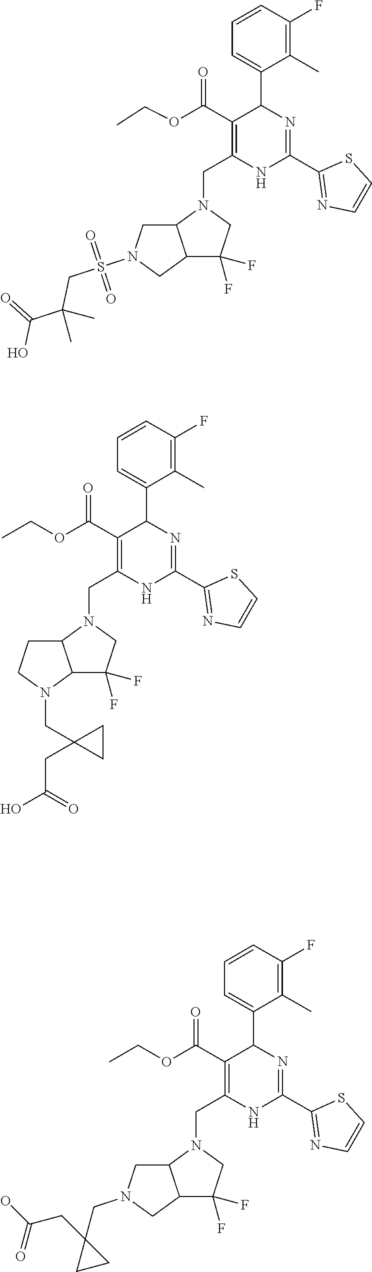 [SMILES: CCOC(=O)C1=C(CN2CC(F)(F)C3CN(S(=O)(=O)CC(C)(C)C(=O)O)CC32)NC(c2nccs2)=NC1c1cccc(F)c1C ; CCOC(=O)C1=C(CN2CC(F)(F)C3C2CCN3CC2(CC(=O)O)CC2)NC(c2nccs2)=NC1c1cccc(F)c1C ; CCOC(=O)C1=C(CN2CC(F)(F)C3CN(CC4(CC(=O)O)CC4)CC32)NC(c2nccs2)=NC1c1cccc(F)c1C]

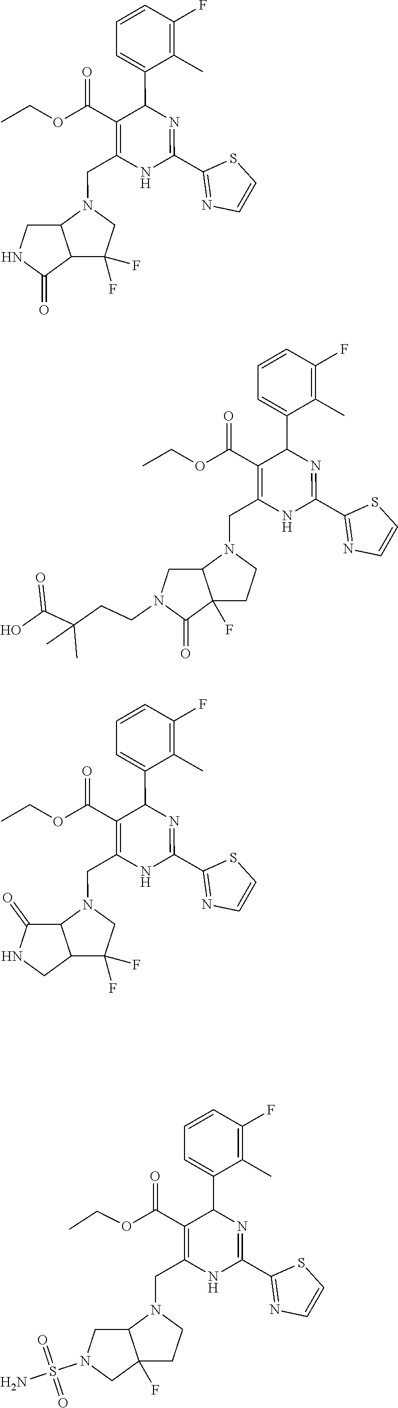 [SMILES: CCOC(=O)C1=C(CN2CC(F)(F)C3C(=O)NCC32)NC(c2nccs2)=NC1c1cccc(F)c1C ; CCOC(=O)C1=C(CN2CCC3(F)C(=O)N(CCC(C)(C)C(=O)O)CC23)NC(c2nccs2)=NC1c1cccc(F)c1C ; CCOC(=O)C1=C(CN2CC(F)(F)C3CNC(=O)C32)NC(c2nccs2)=NC1c1cccc(F)c1C ; CCOC(=O)C1=C(CN2CCC3(F)CN(S(N)(=O)=O)CC23)NC(c2nccs2)=NC1c1cccc(F)c1C]

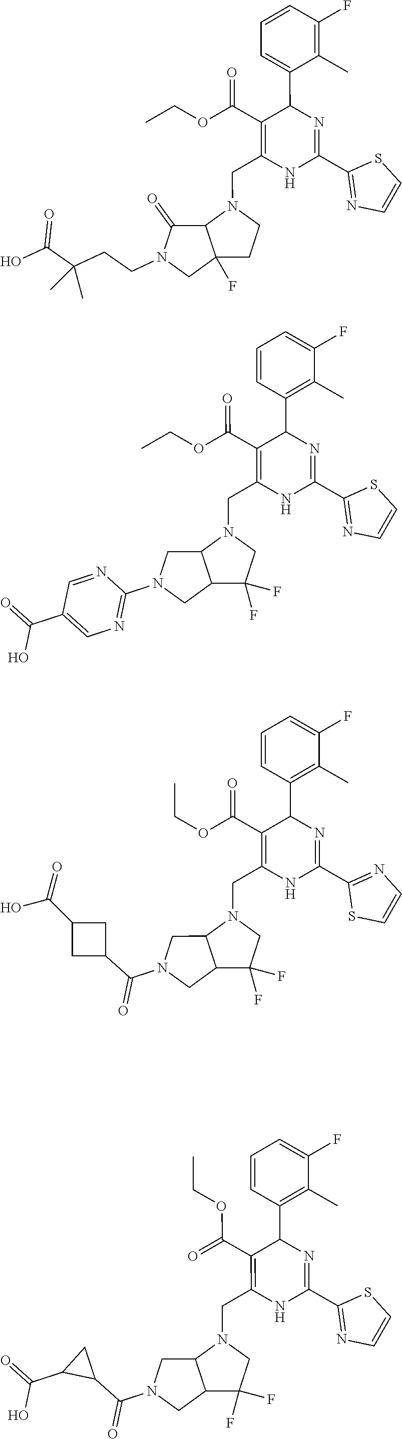 [SMILES: CCOC(=O)C1=C(CN2CCC3(F)CN(CCC(C)(C)C(=O)O)C(=O)C23)NC(c2nccs2)=NC1c1cccc(F)c1C ; CCOC(=O)C1=C(CN2CC(F)(F)C3CN(c4ncc(C(=O)O)cn4)CC32)NC(c2nccs2)=NC1c1cccc(F)c1C ; CCOC(=O)C1=C(CN2CC(F)(F)C3CN(C(=O)C4CC(C(=O)O)C4)CC32)NC(c2nccs2)=NC1c1cccc(F)c1C ; CCOC(=O)C1=C(CN2CC(F)(F)C3CN(C(=O)C4CC4C(=O)O)CC32)NC(c2nccs2)=NC1c1cccc(F)c1C]

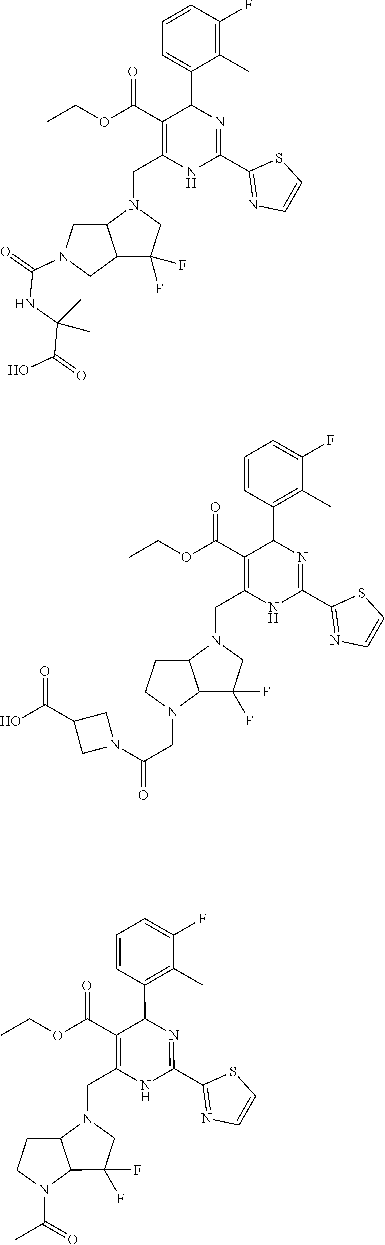 [SMILES: CCOC(=O)C1=C(CN2CC(F)(F)C3CN(C(=O)NC(C)(C)C(=O)O)CC32)NC(c2nccs2)=NC1c1cccc(F)c1C ; CCOC(=O)C1=C(CN2CC(F)(F)C3C2CCN3CC(=O)N2CC(C(=O)O)C2)NC(c2nccs2)=NC1c1cccc(F)c1C ; CCOC(=O)C1=C(CN2CC(F)(F)C3C2CCN3C(C)=O)NC(c2nccs2)=NC1c1cccc(F)c1C]

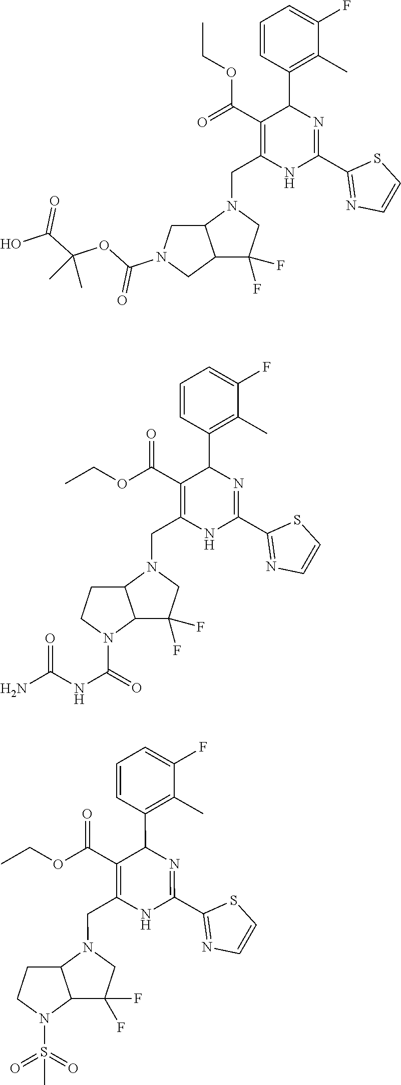 [SMILES: CCOC(=O)C1=C(CN2CC(F)(F)C3CN(C(=O)OC(C)(C)C(=O)O)CC32)NC(c2nccs2)=NC1c1cccc(F)c1C ; CCOC(=O)C1=C(CN2CC(F)(F)C3C2CCN3C(=O)NC(N)=O)NC(c2nccs2)=NC1c1cccc(F)c1C ; CCOC(=O)C1=C(CN2CC(F)(F)C3C2CCN3S(C)(=O)=O)NC(c2nccs2)=NC1c1cccc(F)c1C]

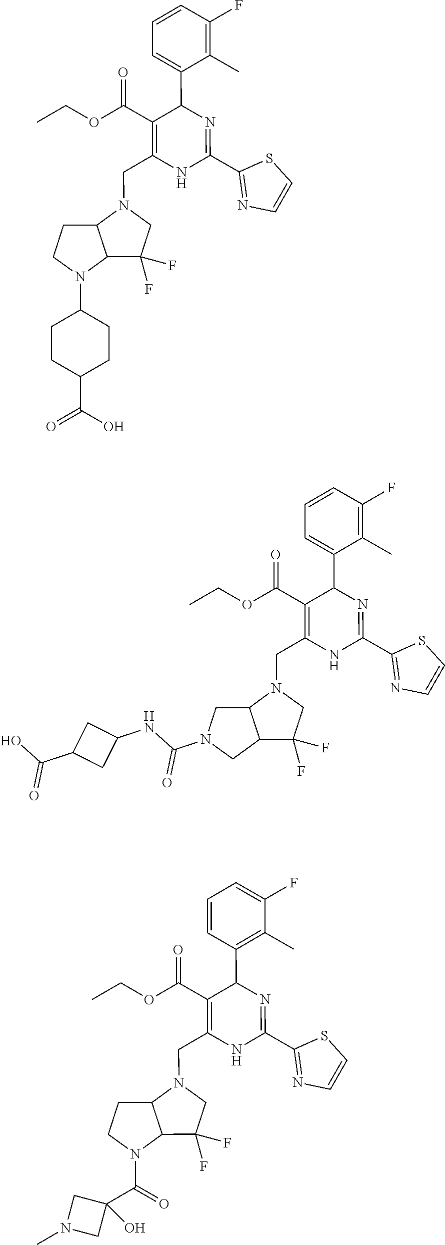 [SMILES: CCOC(=O)C1=C(CN2CC(F)(F)C3C2CCN3C2CCC(C(=O)O)CC2)NC(c2nccs2)=NC1c1cccc(F)c1C ; CCOC(=O)C1=C(CN2CC(F)(F)C3CN(C(=O)NC4CC(C(=O)O)C4)CC32)NC(c2nccs2)=NC1c1cccc(F)c1C ; CCOC(=O)C1=C(CN2CC(F)(F)C3C2CCN3C(=O)C2(O)CN(C)C2)NC(c2nccs2)=NC1c1cccc(F)c1C]

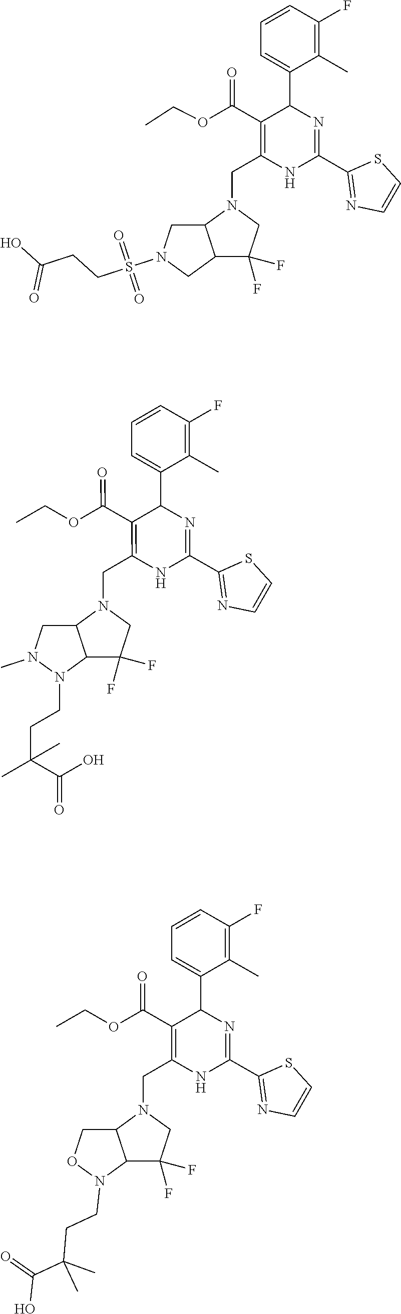 [SMILES: CCOC(=O)C1=C(CN2CC(F)(F)C3CN(S(=O)(=O)CCC(=O)O)CC32)NC(c2nccs2)=NC1c1cccc(F)c1C ; CCOC(=O)C1=C(CN2CC(F)(F)C3C2CN(C)N3CCC(C)(C)C(=O)O)NC(c2nccs2)=NC1c1cccc(F)c1C ; CCOC(=O)C1=C(CN2CC(F)(F)C3C2CON3CCC(C)(C)C(=O)O)NC(c2nccs2)=NC1c1cccc(F)c1C]

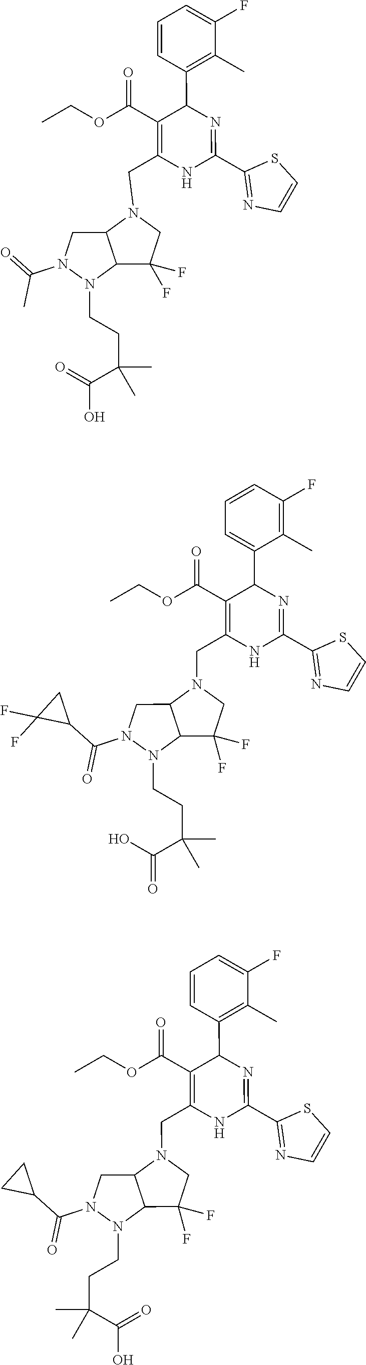 [SMILES: CCOC(=O)C1=C(CN2CC(F)(F)C3C2CN(C(C)=O)N3CCC(C)(C)C(=O)O)NC(c2nccs2)=NC1c1cccc(F)c1C ; CCOC(=O)C1=C(CN2CC(F)(F)C3C2CN(C(=O)C2CC2(F)F)N3CCC(C)(C)C(=O)O)NC(c2nccs2)=NC1c1cccc(F)c1C ; CCOC(=O)C1=C(CN2CC(F)(F)C3C2CN(C(=O)C2CC2)N3CCC(C)(C)C(=O)O)NC(c2nccs2)=NC1c1cccc(F)c1C]

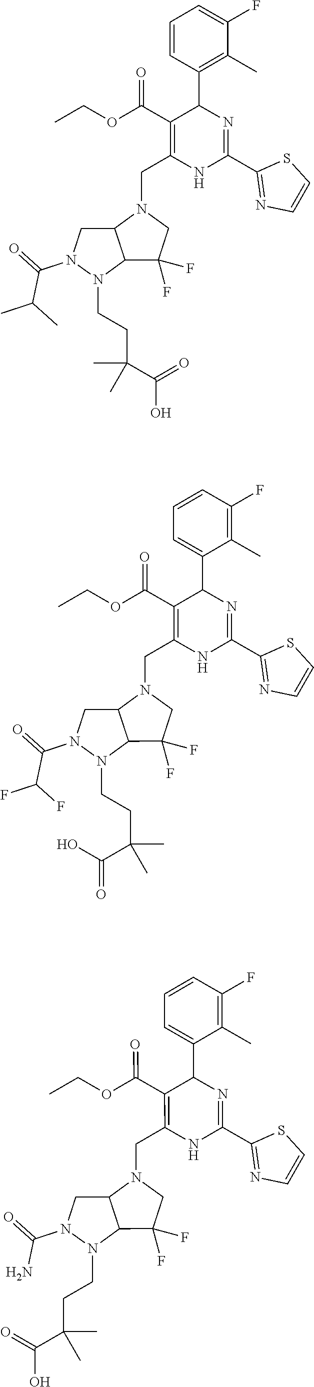 [SMILES: CCOC(=O)C1=C(CN2CC(F)(F)C3C2CN(C(=O)C(C)C)N3CCC(C)(C)C(=O)O)NC(c2nccs2)=NC1c1cccc(F)c1C ; CCOC(=O)C1=C(CN2CC(F)(F)C3C2CN(C(=O)C(F)F)N3CCC(C)(C)C(=O)O)NC(c2nccs2)=NC1c1cccc(F)c1C ; CCOC(=O)C1=C(CN2CC(F)(F)C3C2CN(C(N)=O)N3CCC(C)(C)C(=O)O)NC(c2nccs2)=NC1c1cccc(F)c1C]

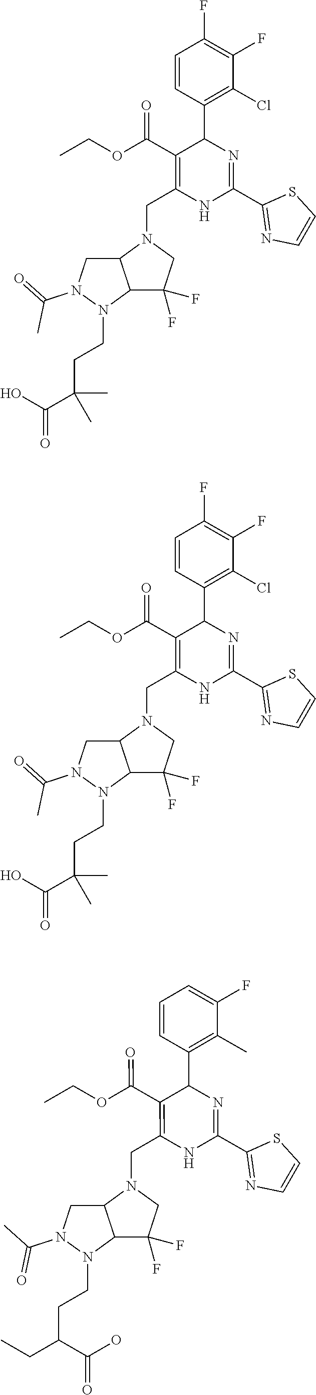 [SMILES: CCOC(=O)C1=C(CN2CC(F)(F)C3C2CN(C(C)=O)N3CCC(C)(C)C(=O)O)NC(c2nccs2)=NC1c1ccc(F)c(F)c1Cl ; CCOC(=O)C1=C(CN2CC(F)(F)C3C2CN(C(C)=O)N3CCC(C)(C)C(=O)O)NC(c2nccs2)=NC1c1ccc(F)c(F)c1Cl ; CCOC(=O)C1=C(CN2CC(F)(F)C3C2CN(C(C)=O)N3CCC(CC)C(=O)O)NC(c2nccs2)=NC1c1cccc(F)c1C]

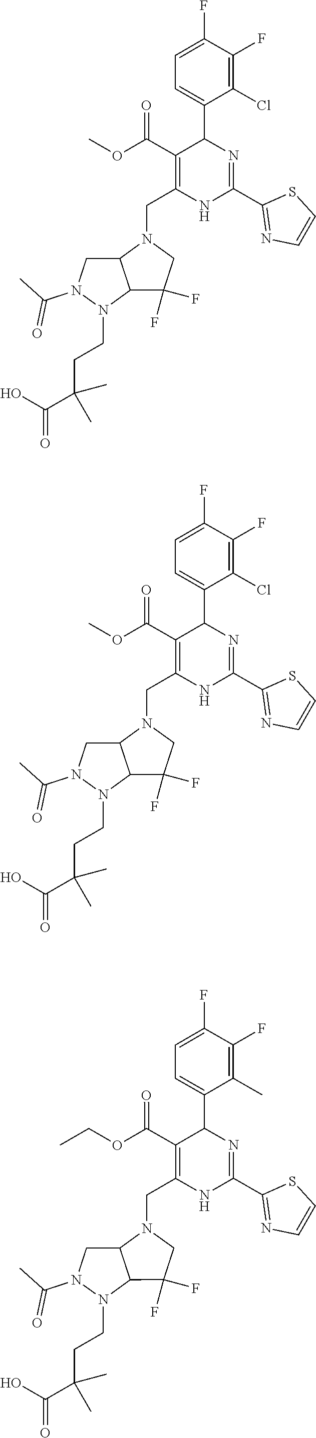 [SMILES: COC(=O)C1=C(CN2CC(F)(F)C3C2CN(C(C)=O)N3CCC(C)(C)C(=O)O)NC(c2nccs2)=NC1c1ccc(F)c(F)c1Cl ; COC(=O)C1=C(CN2CC(F)(F)C3C2CN(C(C)=O)N3CCC(C)(C)C(=O)O)NC(c2nccs2)=NC1c1ccc(F)c(F)c1Cl ; CCOC(=O)C1=C(CN2CC(F)(F)C3C2CN(C(C)=O)N3CCC(C)(C)C(=O)O)NC(c2nccs2)=NC1c1ccc(F)c(F)c1C]

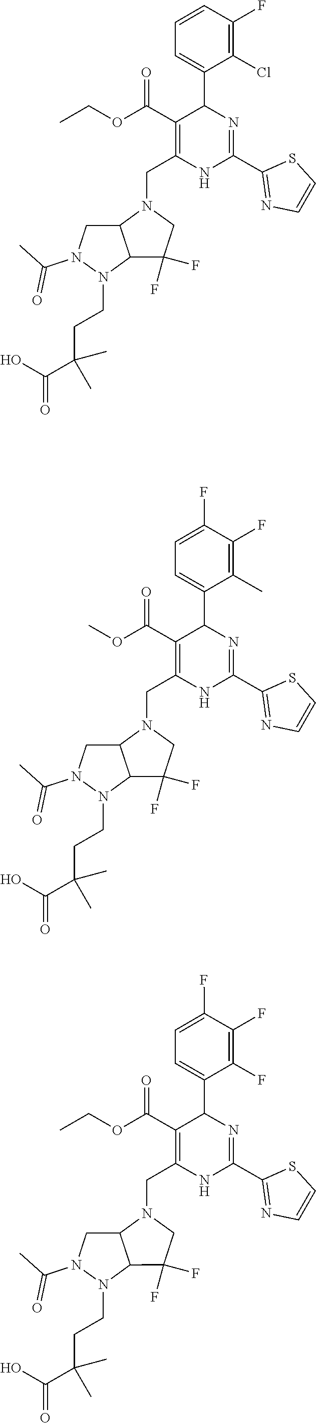 [SMILES: CCOC(=O)C1=C(CN2CC(F)(F)C3C2CN(C(C)=O)N3CCC(C)(C)C(=O)O)NC(c2nccs2)=NC1c1cccc(F)c1Cl ; COC(=O)C1=C(CN2CC(F)(F)C3C2CN(C(C)=O)N3CCC(C)(C)C(=O)O)NC(c2nccs2)=NC1c1ccc(F)c(F)c1C ; CCOC(=O)C1=C(CN2CC(F)(F)C3C2CN(C(C)=O)N3CCC(C)(C)C(=O)O)NC(c2nccs2)=NC1c1ccc(F)c(F)c1F]

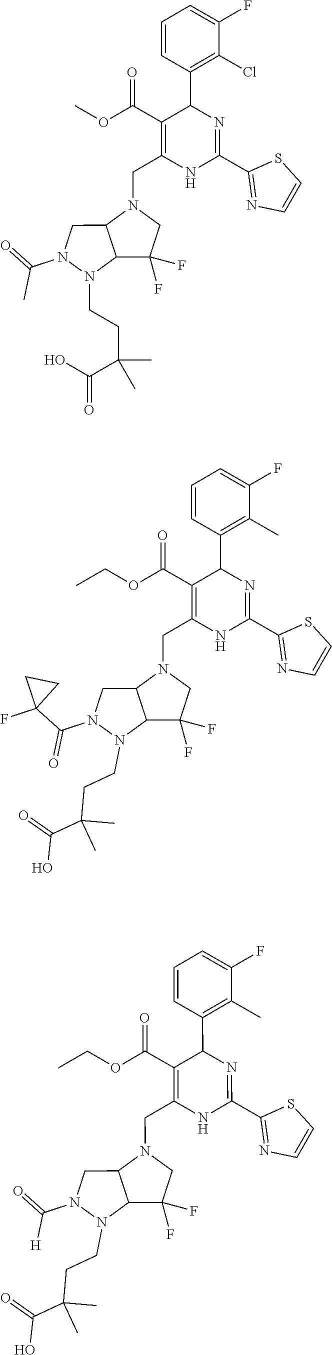 [SMILES: COC(=O)C1=C(CN2CC(F)(F)C3C2CN(C(C)=O)N3CCC(C)(C)C(=O)O)NC(c2nccs2)=NC1c1cccc(F)c1Cl ; CCOC(=O)C1=C(CN2CC(F)(F)C3C2CN(C(=O)C2(F)CC2)N3CCC(C)(C)C(=O)O)NC(c2nccs2)=NC1c1cccc(F)c1C ; CCOC(=O)C1=C(CN2CC(F)(F)C3C2CN(C=O)N3CCC(C)(C)C(=O)O)NC(c2nccs2)=NC1c1cccc(F)c1C]

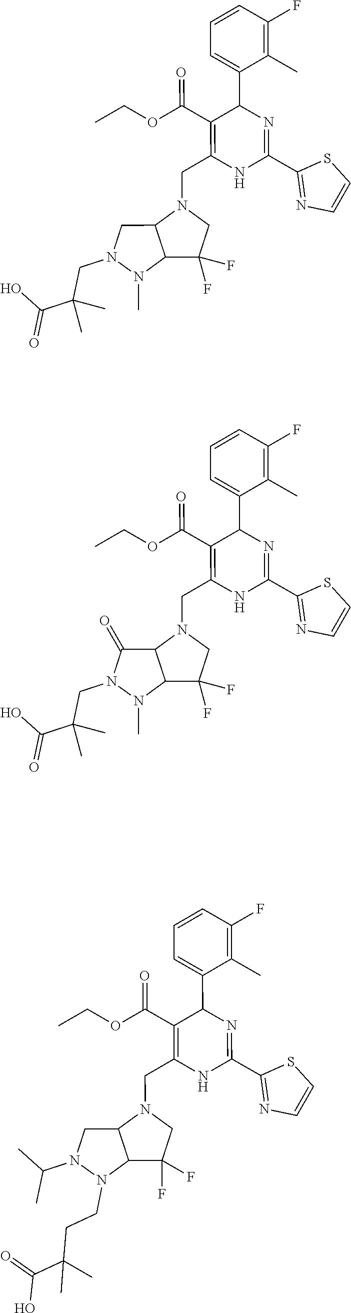 [SMILES: CCOC(=O)C1=C(CN2CC(F)(F)C3C2CN(CC(C)(C)C(=O)O)N3C)NC(c2nccs2)=NC1c1cccc(F)c1C ; CCOC(=O)C1=C(CN2CC(F)(F)C3C2C(=O)N(CC(C)(C)C(=O)O)N3C)NC(c2nccs2)=NC1c1cccc(F)c1C ; CCOC(=O)C1=C(CN2CC(F)(F)C3C2CN(C(C)C)N3CCC(C)(C)C(=O)O)NC(c2nccs2)=NC1c1cccc(F)c1C]

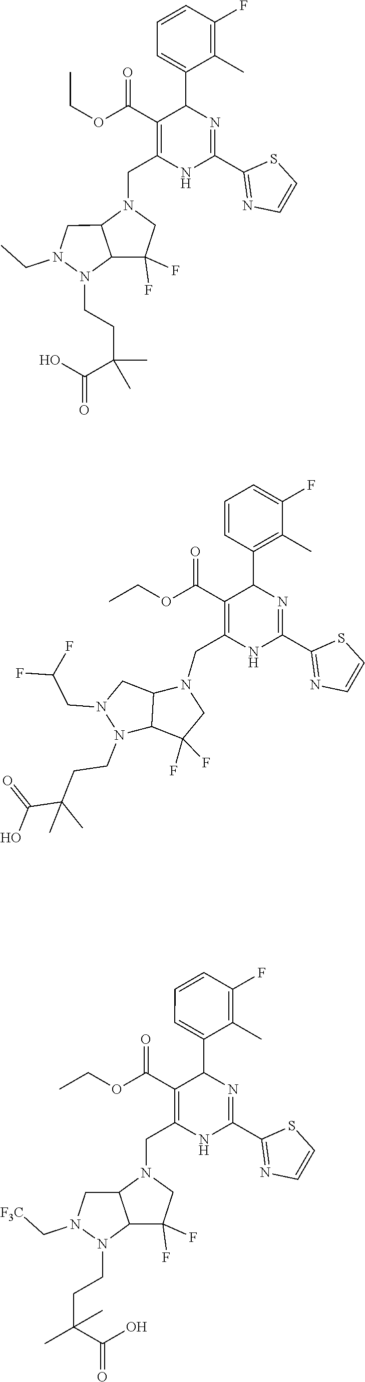 [SMILES: CCOC(=O)C1=C(CN2CC(F)(F)C3C2CN(CC)N3CCC(C)(C)C(=O)O)NC(c2nccs2)=NC1c1cccc(F)c1C ; CCOC(=O)C1=C(CN2CC(F)(F)C3C2CN(CC(F)F)N3CCC(C)(C)C(=O)O)NC(c2nccs2)=NC1c1cccc(F)c1C ; CCOC(=O)C1=C(CN2CC(F)(F)C3C2CN(CC(F)(F)F)N3CCC(C)(C)C(=O)O)NC(c2nccs2)=NC1c1cccc(F)c1C]

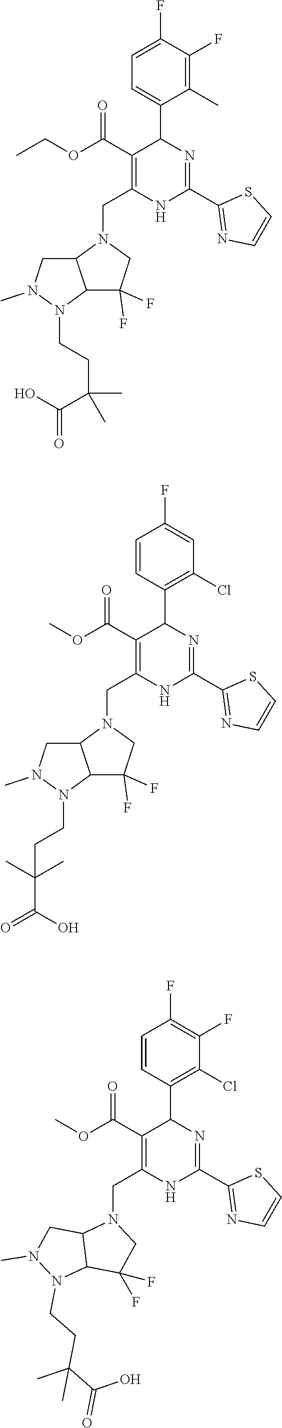 [SMILES: CCOC(=O)C1=C(CN2CC(F)(F)C3C2CN(C)N3CCC(C)(C)C(=O)O)NC(c2nccs2)=NC1c1ccc(F)c(F)c1C ; COC(=O)C1=C(CN2CC(F)(F)C3C2CN(C)N3CCC(C)(C)C(=O)O)NC(c2nccs2)=NC1c1ccc(F)cc1Cl ; COC(=O)C1=C(CN2CC(F)(F)C3C2CN(C)N3CCC(C)(C)C(=O)O)NC(c2nccs2)=NC1c1ccc(F)c(F)c1Cl]

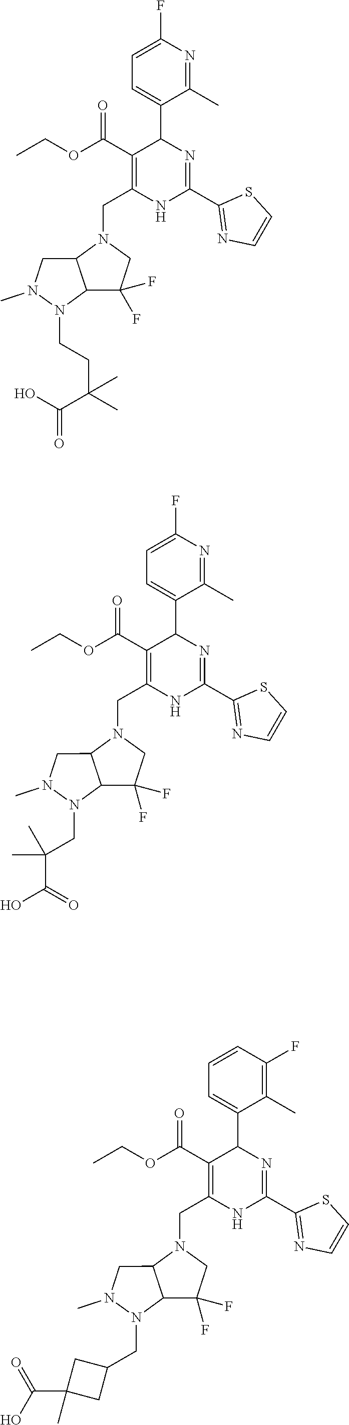 [SMILES: CCOC(=O)C1=C(CN2CC(F)(F)C3C2CN(C)N3CCC(C)(C)C(=O)O)NC(c2nccs2)=NC1c1ccc(F)nc1C ; CCOC(=O)C1=C(CN2CC(F)(F)C3C2CN(C)N3CC(C)(C)C(=O)O)NC(c2nccs2)=NC1c1ccc(F)nc1C ; CCOC(=O)C1=C(CN2CC(F)(F)C3C2CN(C)N3CC2CC(C)(C(=O)O)C2)NC(c2nccs2)=NC1c1cccc(F)c1C]

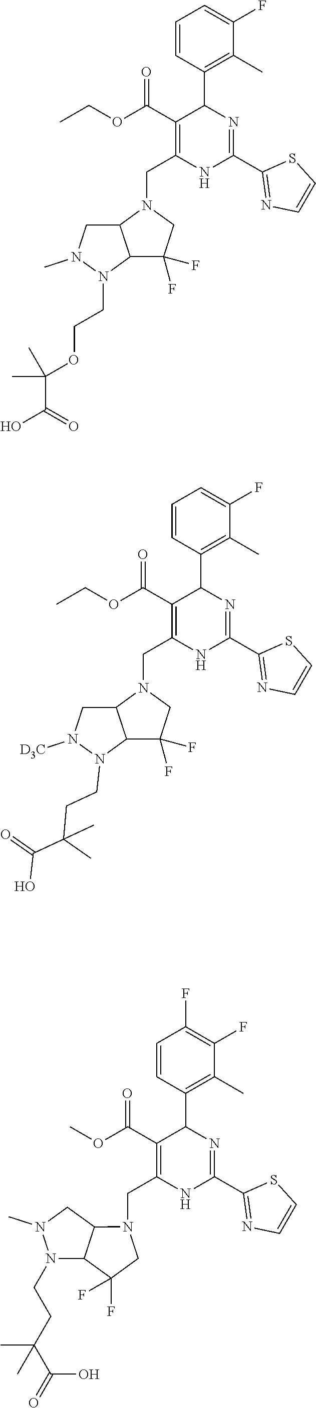 [SMILES: CCOC(=O)C1=C(CN2CC(F)(F)C3C2CN(C)N3CCOC(C)(C)C(=O)O)NC(c2nccs2)=NC1c1cccc(F)c1C ; [2H]C([2H])([2H])N1CC2C(N1CCC(C)(C)C(=O)O)C(F)(F)CN2CC1=C(C(=O)OCC)C(c2cccc(F)c2C)N=C(c2nccs2)N1 ; COC(=O)C1=C(CN2CC(F)(F)C3C2CN(C)N3CCC(C)(C)C(=O)O)NC(c2nccs2)=NC1c1ccc(F)c(F)c1C]

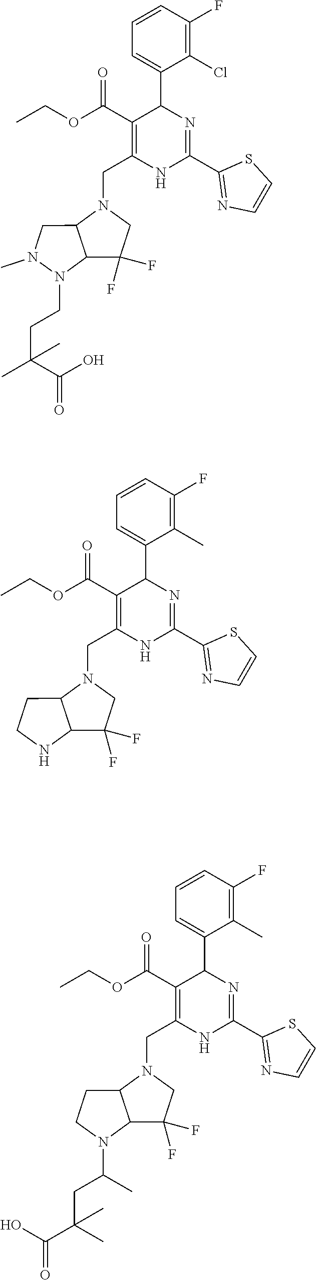 [SMILES: CCOC(=O)C1=C(CN2CC(F)(F)C3C2CN(C)N3CCC(C)(C)C(=O)O)NC(c2nccs2)=NC1c1cccc(F)c1Cl ; CCOC(=O)C1=C(CN2CC(F)(F)C3NCCC32)NC(c2nccs2)=NC1c1cccc(F)c1C ; CCOC(=O)C1=C(CN2CC(F)(F)C3C2CCN3C(C)CC(C)(C)C(=O)O)NC(c2nccs2)=NC1c1cccc(F)c1C]

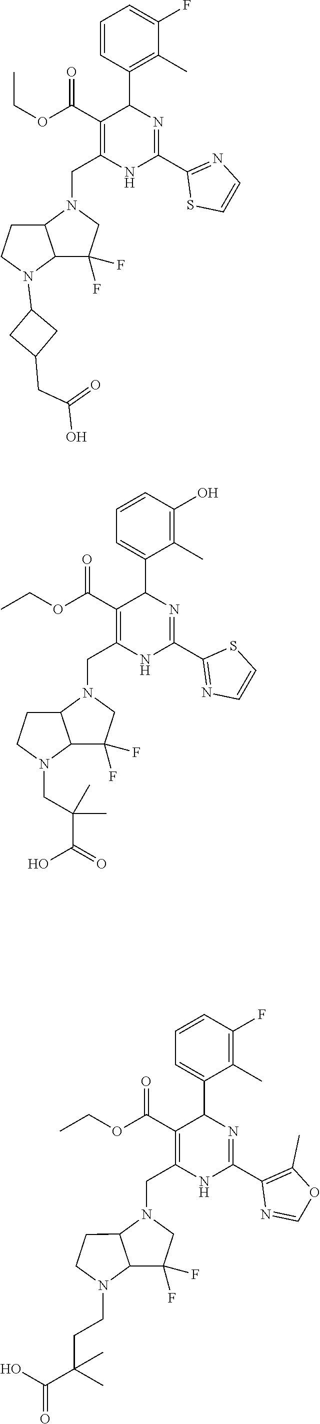 [SMILES: CCOC(=O)C1=C(CN2CC(F)(F)C3C2CCN3C2CC(CC(=O)O)C2)NC(c2nccs2)=NC1c1cccc(F)c1C ; CCOC(=O)C1=C(CN2CC(F)(F)C3C2CCN3CC(C)(C)C(=O)O)NC(c2nccs2)=NC1c1cccc(O)c1C ; CCOC(=O)C1=C(CN2CC(F)(F)C3C2CCN3CCC(C)(C)C(=O)O)NC(c2ncoc2C)=NC1c1cccc(F)c1C]

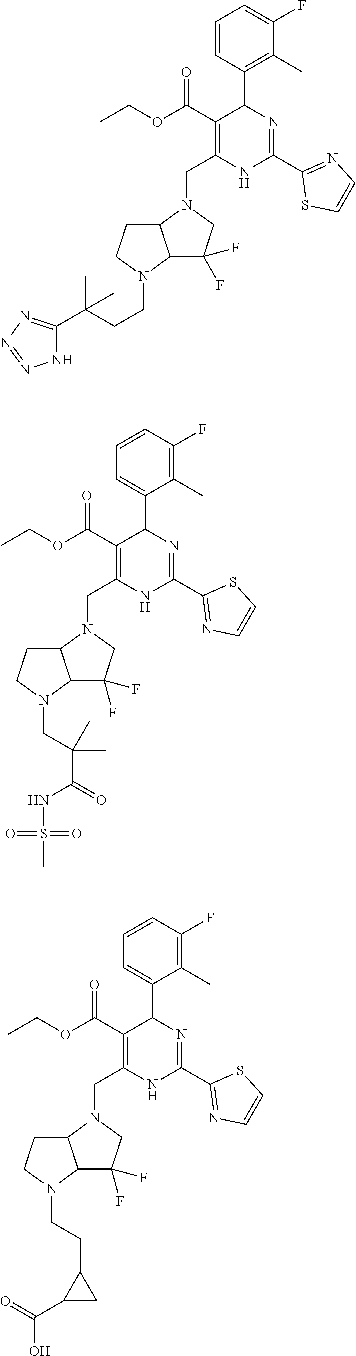 [SMILES: CCOC(=O)C1=C(CN2CC(F)(F)C3C2CCN3CCC(C)(C)c2nnn[nH]2)NC(c2nccs2)=NC1c1cccc(F)c1C ; CCOC(=O)C1=C(CN2CC(F)(F)C3C2CCN3CC(C)(C)C(=O)NS(C)(=O)=O)NC(c2nccs2)=NC1c1cccc(F)c1C ; CCOC(=O)C1=C(CN2CC(F)(F)C3C2CCN3CCC2CC2C(=O)O)NC(c2nccs2)=NC1c1cccc(F)c1C]

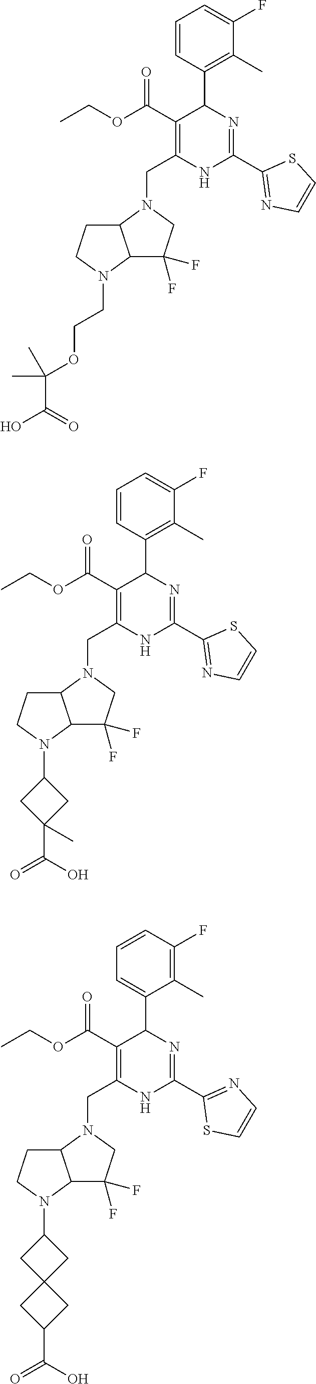 [SMILES: CCOC(=O)C1=C(CN2CC(F)(F)C3C2CCN3CCOC(C)(C)C(=O)O)NC(c2nccs2)=NC1c1cccc(F)c1C ; CCOC(=O)C1=C(CN2CC(F)(F)C3C2CCN3C2CC(C)(C(=O)O)C2)NC(c2nccs2)=NC1c1cccc(F)c1C ; CCOC(=O)C1=C(CN2CC(F)(F)C3C2CCN3C2CC3(CC(C(=O)O)C3)C2)NC(c2nccs2)=NC1c1cccc(F)c1C]

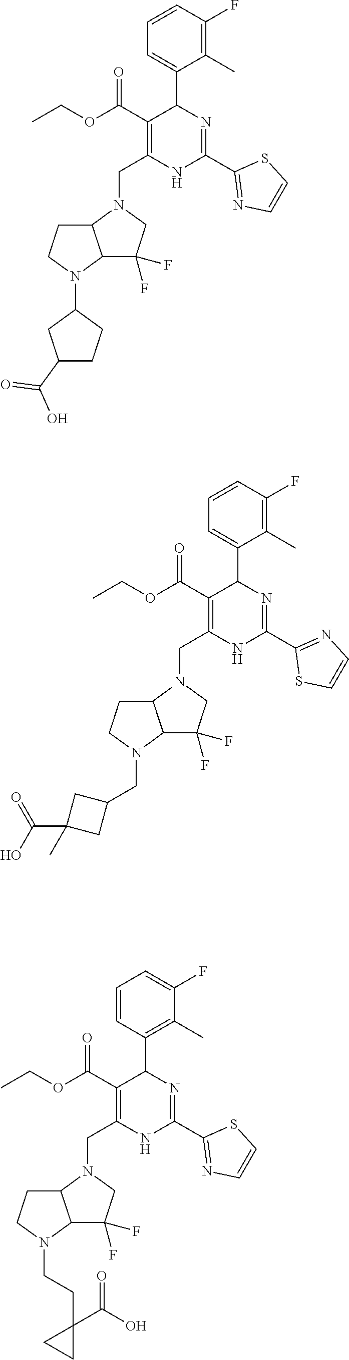 [SMILES: CCOC(=O)C1=C(CN2CC(F)(F)C3C2CCN3C2CCC(C(=O)O)C2)NC(c2nccs2)=NC1c1cccc(F)c1C ; CCOC(=O)C1=C(CN2CC(F)(F)C3C2CCN3CC2CC(C)(C(=O)O)C2)NC(c2nccs2)=NC1c1cccc(F)c1C ; CCOC(=O)C1=C(CN2CC(F)(F)C3C2CCN3CCC2(C(=O)O)CC2)NC(c2nccs2)=NC1c1cccc(F)c1C]

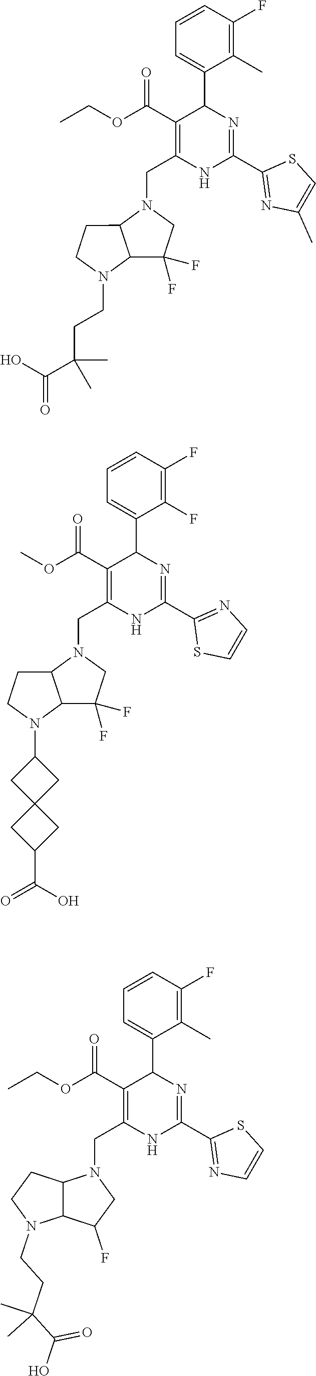 [SMILES: CCOC(=O)C1=C(CN2CC(F)(F)C3C2CCN3CCC(C)(C)C(=O)O)NC(c2nc(C)cs2)=NC1c1cccc(F)c1C ; COC(=O)C1=C(CN2CC(F)(F)C3C2CCN3C2CC3(CC(C(=O)O)C3)C2)NC(c2nccs2)=NC1c1cccc(F)c1F ; CCOC(=O)C1=C(CN2CC(F)C3C2CCN3CCC(C)(C)C(=O)O)NC(c2nccs2)=NC1c1cccc(F)c1C]

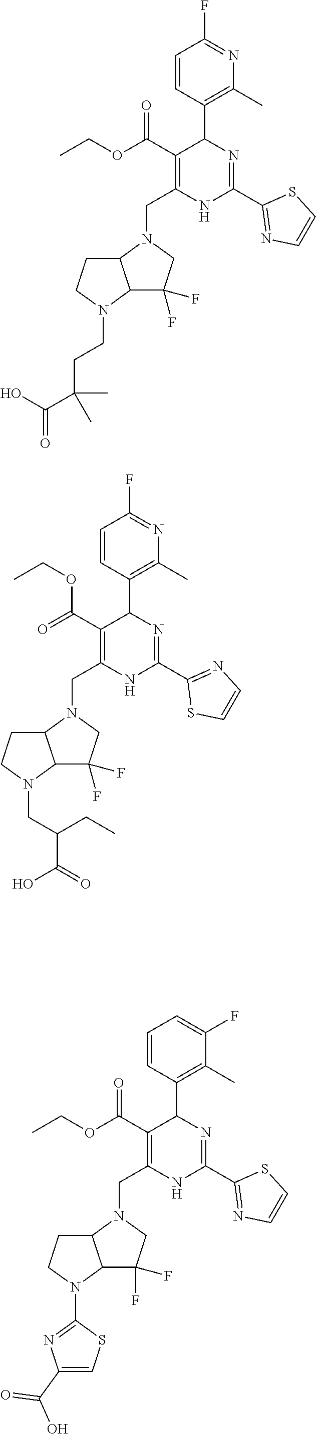 [SMILES: CCOC(=O)C1=C(CN2CC(F)(F)C3C2CCN3CCC(C)(C)C(=O)O)NC(c2nccs2)=NC1c1ccc(F)nc1C ; CCOC(=O)C1=C(CN2CC(F)(F)C3C2CCN3CC(CC)C(=O)O)NC(c2nccs2)=NC1c1ccc(F)nc1C ; CCOC(=O)C1=C(CN2CC(F)(F)C3C2CCN3c2nc(C(=O)O)cs2)NC(c2nccs2)=NC1c1cccc(F)c1C]

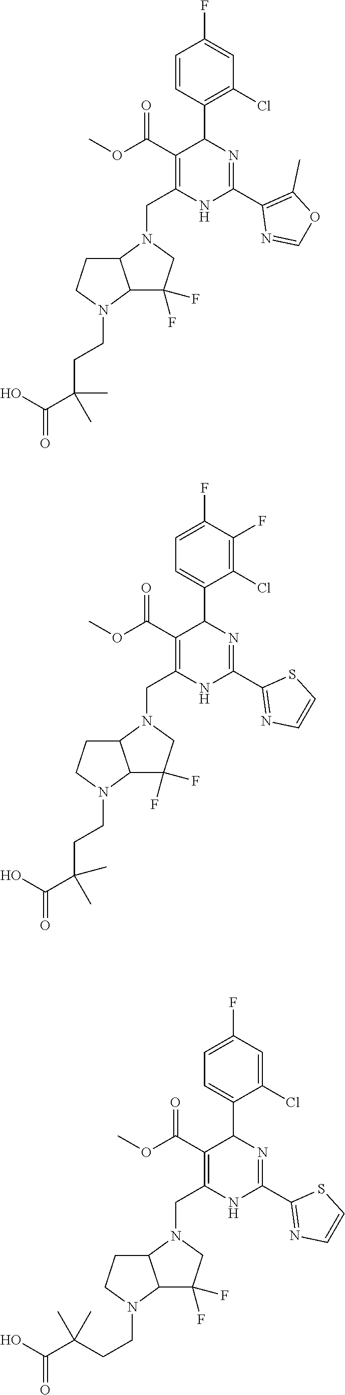 [SMILES: COC(=O)C1=C(CN2CC(F)(F)C3C2CCN3CCC(C)(C)C(=O)O)NC(c2ncoc2C)=NC1c1ccc(F)cc1Cl ; COC(=O)C1=C(CN2CC(F)(F)C3C2CCN3CCC(C)(C)C(=O)O)NC(c2nccs2)=NC1c1ccc(F)c(F)c1Cl ; COC(=O)C1=C(CN2CC(F)(F)C3C2CCN3CCC(C)(C)C(=O)O)NC(c2nccs2)=NC1c1ccc(F)cc1Cl]

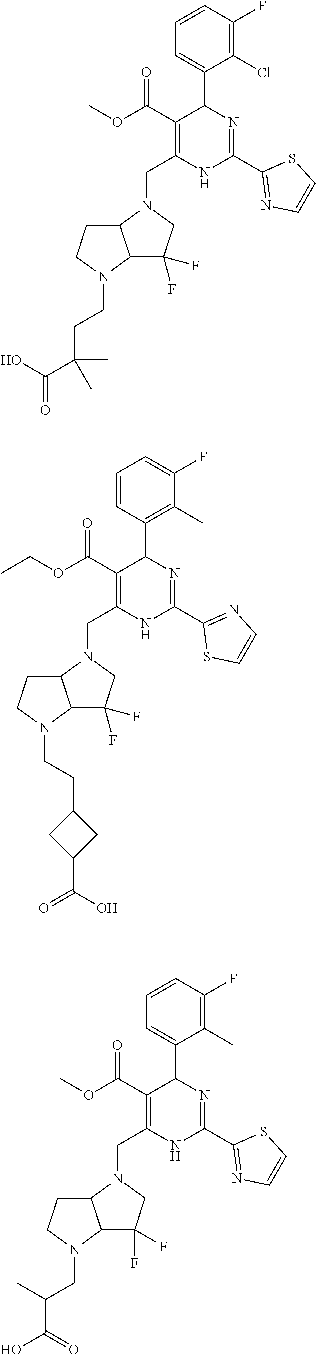 [SMILES: COC(=O)C1=C(CN2CC(F)(F)C3C2CCN3CCC(C)(C)C(=O)O)NC(c2nccs2)=NC1c1cccc(F)c1Cl ; CCOC(=O)C1=C(CN2CC(F)(F)C3C2CCN3CCC2CC(C(=O)O)C2)NC(c2nccs2)=NC1c1cccc(F)c1C ; COC(=O)C1=C(CN2CC(F)(F)C3C2CCN3CC(C)C(=O)O)NC(c2nccs2)=NC1c1cccc(F)c1C]

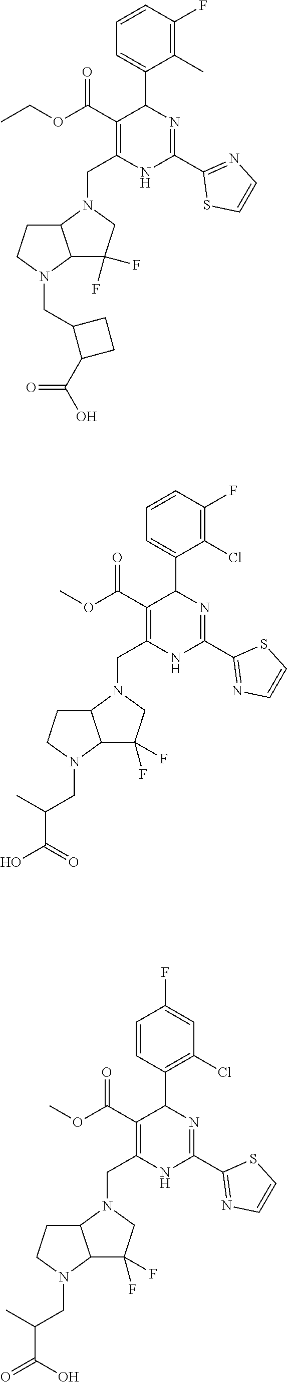 [SMILES: CCOC(=O)C1=C(CN2CC(F)(F)C3C2CCN3CC2CCC2C(=O)O)NC(c2nccs2)=NC1c1cccc(F)c1C ; COC(=O)C1=C(CN2CC(F)(F)C3C2CCN3CC(C)C(=O)O)NC(c2nccs2)=NC1c1cccc(F)c1Cl ; COC(=O)C1=C(CN2CC(F)(F)C3C2CCN3CC(C)C(=O)O)NC(c2nccs2)=NC1c1ccc(F)cc1Cl]

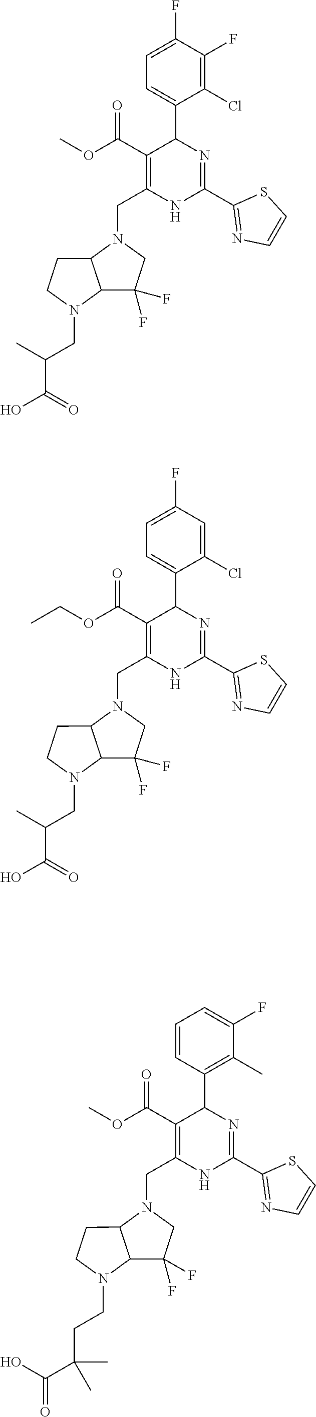 [SMILES: COC(=O)C1=C(CN2CC(F)(F)C3C2CCN3CC(C)C(=O)O)NC(c2nccs2)=NC1c1ccc(F)c(F)c1Cl ; CCOC(=O)C1=C(CN2CC(F)(F)C3C2CCN3CC(C)C(=O)O)NC(c2nccs2)=NC1c1ccc(F)cc1Cl ; COC(=O)C1=C(CN2CC(F)(F)C3C2CCN3CCC(C)(C)C(=O)O)NC(c2nccs2)=NC1c1cccc(F)c1C]

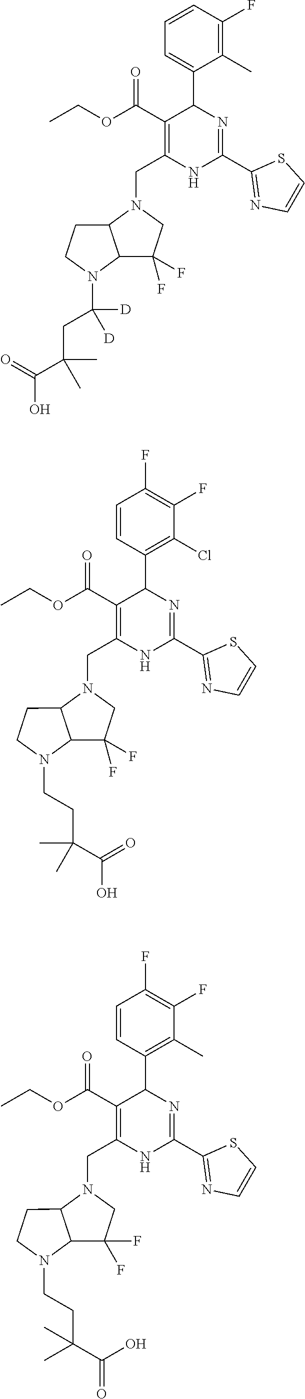 [SMILES: [2H]C([2H])(CC(C)(C)C(=O)O)N1CCC2C1C(F)(F)CN2CC1=C(C(=O)OCC)C(c2cccc(F)c2C)N=C(c2nccs2)N1 ; CCOC(=O)C1=C(CN2CC(F)(F)C3C2CCN3CCC(C)(C)C(=O)O)NC(c2nccs2)=NC1c1ccc(F)c(F)c1Cl ; CCOC(=O)C1=C(CN2CC(F)(F)C3C2CCN3CCC(C)(C)C(=O)O)NC(c2nccs2)=NC1c1ccc(F)c(F)c1C]

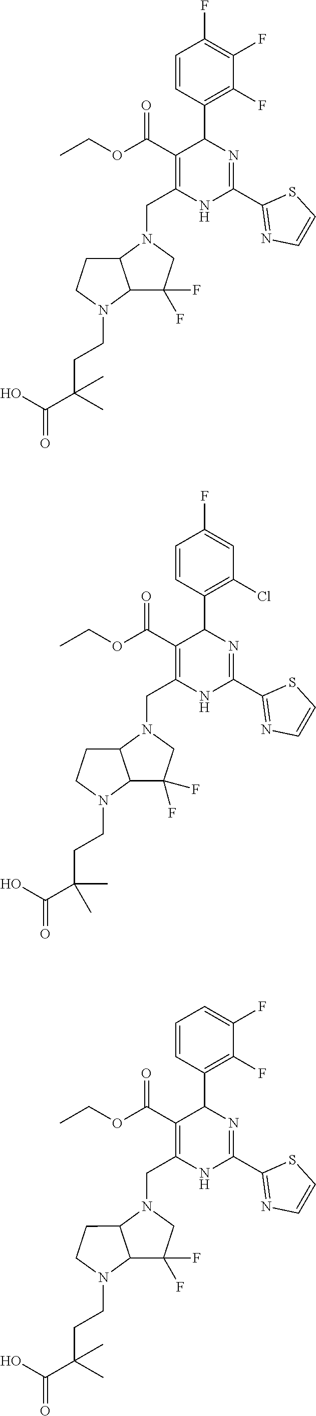 [SMILES: CCOC(=O)C1=C(CN2CC(F)(F)C3C2CCN3CCC(C)(C)C(=O)O)NC(c2nccs2)=NC1c1ccc(F)c(F)c1F ; CCOC(=O)C1=C(CN2CC(F)(F)C3C2CCN3CCC(C)(C)C(=O)O)NC(c2nccs2)=NC1c1ccc(F)cc1Cl ; CCOC(=O)C1=C(CN2CC(F)(F)C3C2CCN3CCC(C)(C)C(=O)O)NC(c2nccs2)=NC1c1cccc(F)c1F]

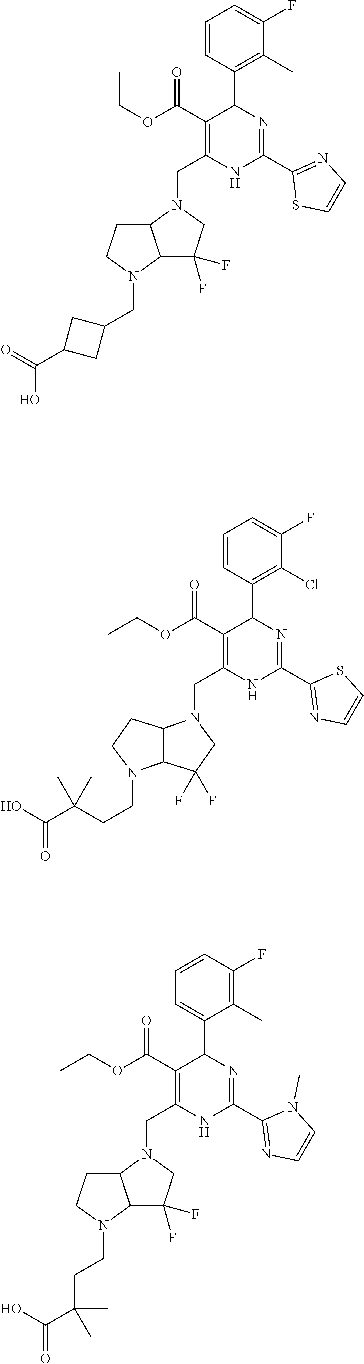 [SMILES: CCOC(=O)C1=C(CN2CC(F)(F)C3C2CCN3CC2CC(C(=O)O)C2)NC(c2nccs2)=NC1c1cccc(F)c1C ; CCOC(=O)C1=C(CN2CC(F)(F)C3C2CCN3CCC(C)(C)C(=O)O)NC(c2nccs2)=NC1c1cccc(F)c1Cl ; CCOC(=O)C1=C(CN2CC(F)(F)C3C2CCN3CCC(C)(C)C(=O)O)NC(c2nccn2C)=NC1c1cccc(F)c1C]

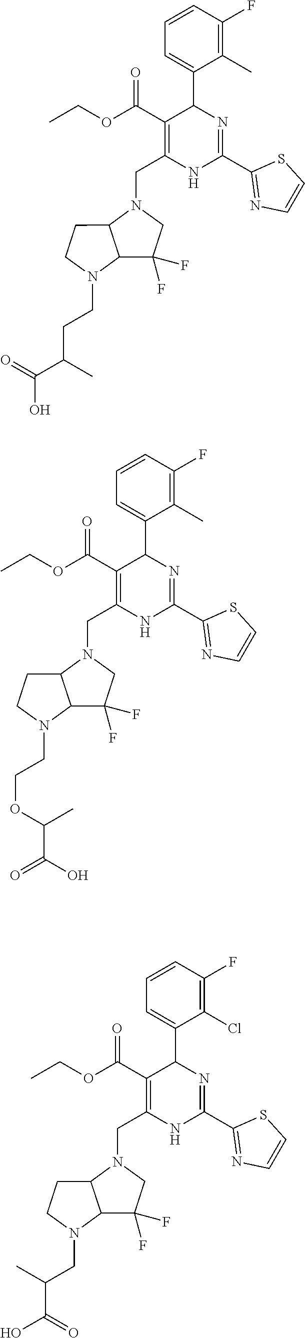 [SMILES: CCOC(=O)C1=C(CN2CC(F)(F)C3C2CCN3CCC(C)C(=O)O)NC(c2nccs2)=NC1c1cccc(F)c1C ; CCOC(=O)C1=C(CN2CC(F)(F)C3C2CCN3CCOC(C)C(=O)O)NC(c2nccs2)=NC1c1cccc(F)c1C ; CCOC(=O)C1=C(CN2CC(F)(F)C3C2CCN3CC(C)C(=O)O)NC(c2nccs2)=NC1c1cccc(F)c1Cl]

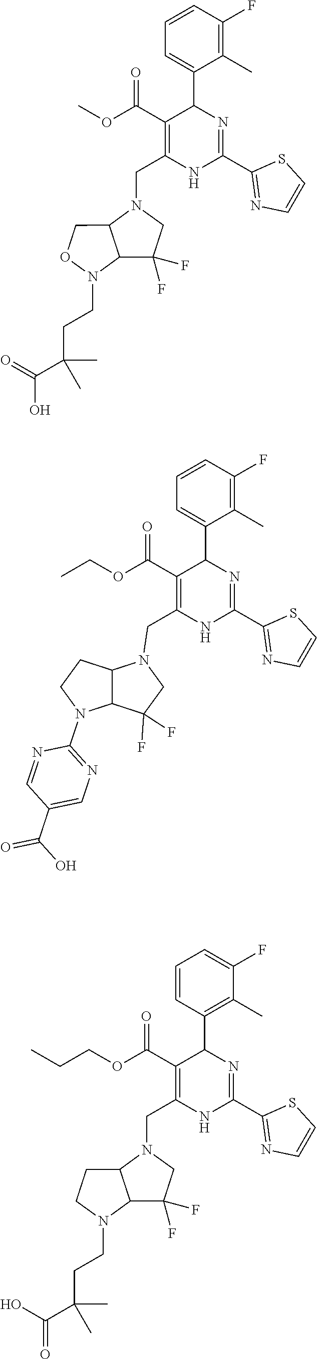 [SMILES: COC(=O)C1=C(CN2CC(F)(F)C3C2CON3CCC(C)(C)C(=O)O)NC(c2nccs2)=NC1c1cccc(F)c1C ; CCOC(=O)C1=C(CN2CC(F)(F)C3C2CCN3c2ncc(C(=O)O)cn2)NC(c2nccs2)=NC1c1cccc(F)c1C ; CCCOC(=O)C1=C(CN2CC(F)(F)C3C2CCN3CCC(C)(C)C(=O)O)NC(c2nccs2)=NC1c1cccc(F)c1C]

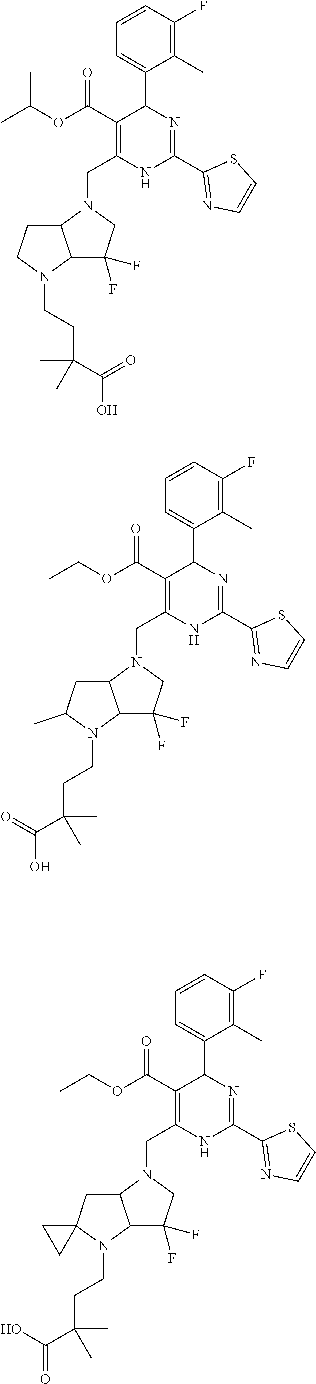 [SMILES: Cc1c(F)cccc1C1N=C(c2nccs2)NC(CN2CC(F)(F)C3C2CCN3CCC(C)(C)C(=O)O)=C1C(=O)OC(C)C ; CCOC(=O)C1=C(CN2CC(F)(F)C3C2CC(C)N3CCC(C)(C)C(=O)O)NC(c2nccs2)=NC1c1cccc(F)c1C ; CCOC(=O)C1=C(CN2CC(F)(F)C3C2CC2(CC2)N3CCC(C)(C)C(=O)O)NC(c2nccs2)=NC1c1cccc(F)c1C]

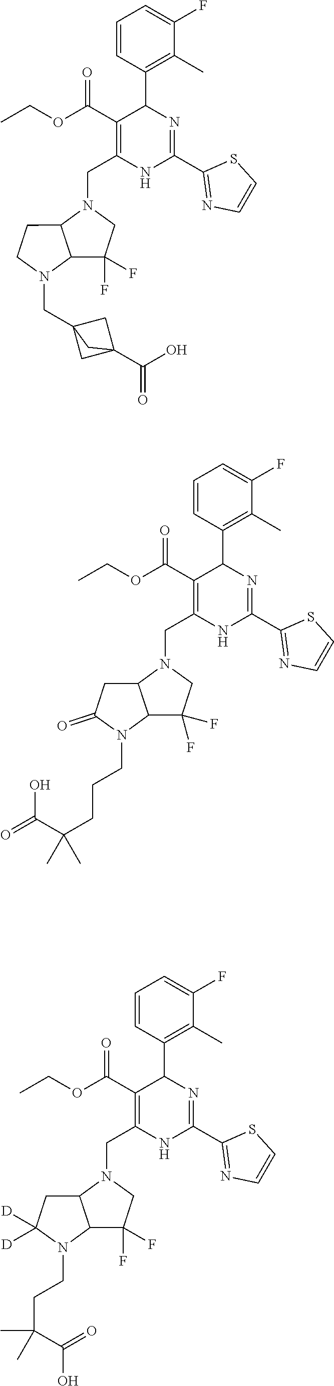 [SMILES: CCOC(=O)C1=C(CN2CC(F)(F)C3C2CCN3CC23CC(C(=O)O)(C2)C3)NC(c2nccs2)=NC1c1cccc(F)c1C ; CCOC(=O)C1=C(CN2CC(F)(F)C3C2CC(=O)N3CCCC(C)(C)C(=O)O)NC(c2nccs2)=NC1c1cccc(F)c1C ; [2H]C1([2H])CC2C(N1CCC(C)(C)C(=O)O)C(F)(F)CN2CC1=C(C(=O)OCC)C(c2cccc(F)c2C)N=C(c2nccs2)N1]

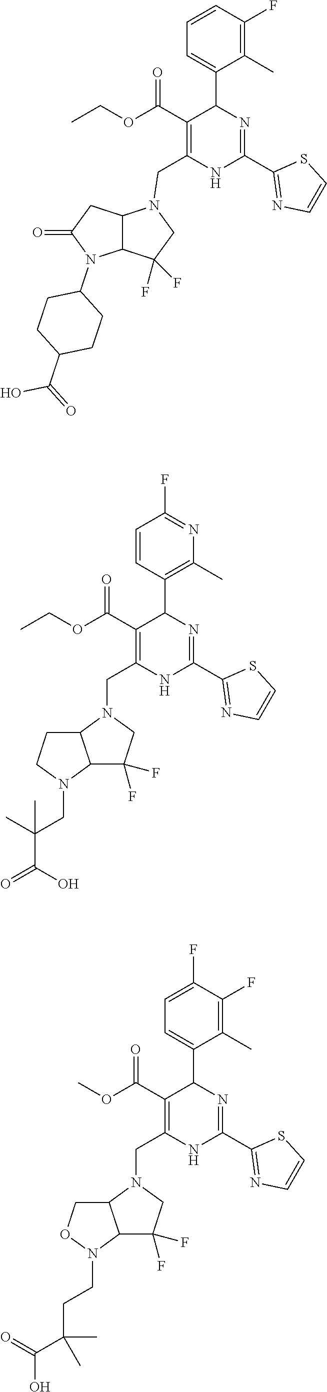 [SMILES: CCOC(=O)C1=C(CN2CC(F)(F)C3C2CC(=O)N3C2CCC(C(=O)O)CC2)NC(c2nccs2)=NC1c1cccc(F)c1C ; CCOC(=O)C1=C(CN2CC(F)(F)C3C2CCN3CC(C)(C)C(=O)O)NC(c2nccs2)=NC1c1ccc(F)nc1C ; COC(=O)C1=C(CN2CC(F)(F)C3C2CON3CCC(C)(C)C(=O)O)NC(c2nccs2)=NC1c1ccc(F)c(F)c1C]

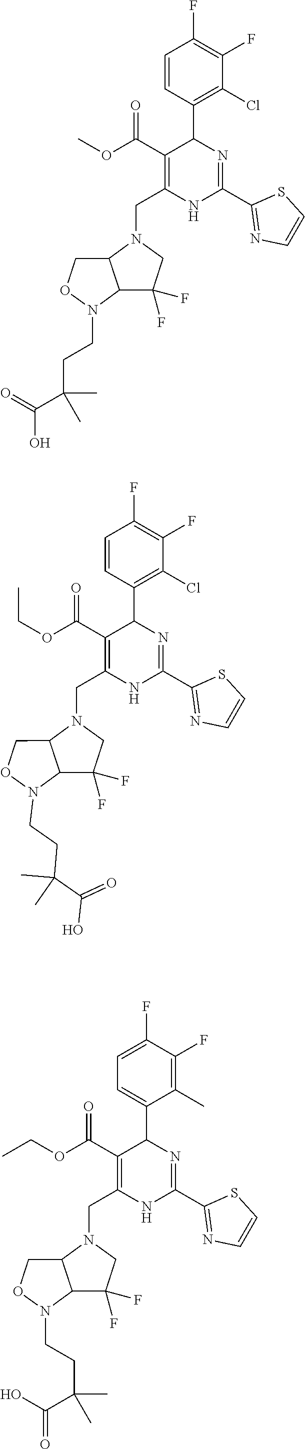 [SMILES: COC(=O)C1=C(CN2CC(F)(F)C3C2CON3CCC(C)(C)C(=O)O)NC(c2nccs2)=NC1c1ccc(F)c(F)c1Cl ; CCOC(=O)C1=C(CN2CC(F)(F)C3C2CON3CCC(C)(C)C(=O)O)NC(c2nccs2)=NC1c1ccc(F)c(F)c1Cl ; CCOC(=O)C1=C(CN2CC(F)(F)C3C2CON3CCC(C)(C)C(=O)O)NC(c2nccs2)=NC1c1ccc(F)c(F)c1C]

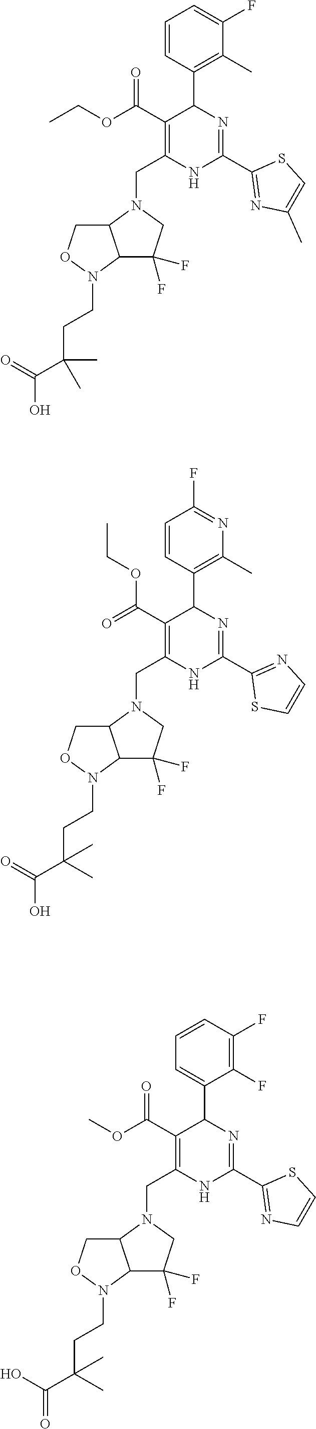 [SMILES: CCOC(=O)C1=C(CN2CC(F)(F)C3C2CON3CCC(C)(C)C(=O)O)NC(c2nc(C)cs2)=NC1c1cccc(F)c1C ; CCOC(=O)C1=C(CN2CC(F)(F)C3C2CON3CCC(C)(C)C(=O)O)NC(c2nccs2)=NC1c1ccc(F)nc1C ; COC(=O)C1=C(CN2CC(F)(F)C3C2CON3CCC(C)(C)C(=O)O)NC(c2nccs2)=NC1c1cccc(F)c1F]

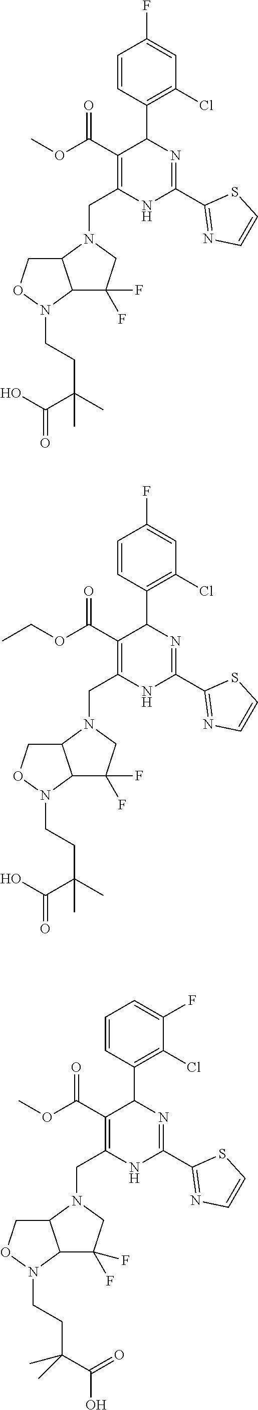 [SMILES: COC(=O)C1=C(CN2CC(F)(F)C3C2CON3CCC(C)(C)C(=O)O)NC(c2nccs2)=NC1c1ccc(F)cc1Cl ; CCOC(=O)C1=C(CN2CC(F)(F)C3C2CON3CCC(C)(C)C(=O)O)NC(c2nccs2)=NC1c1ccc(F)cc1Cl ; COC(=O)C1=C(CN2CC(F)(F)C3C2CON3CCC(C)(C)C(=O)O)NC(c2nccs2)=NC1c1cccc(F)c1Cl]

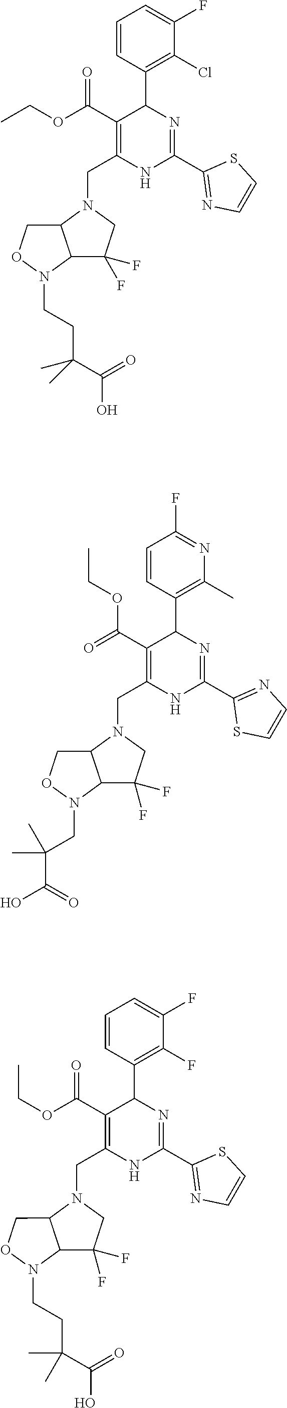 [SMILES: CCOC(=O)C1=C(CN2CC(F)(F)C3C2CON3CCC(C)(C)C(=O)O)NC(c2nccs2)=NC1c1cccc(F)c1Cl ; CCOC(=O)C1=C(CN2CC(F)(F)C3C2CON3CC(C)(C)C(=O)O)NC(c2nccs2)=NC1c1ccc(F)nc1C ; CCOC(=O)C1=C(CN2CC(F)(F)C3C2CON3CCC(C)(C)C(=O)O)NC(c2nccs2)=NC1c1cccc(F)c1F]

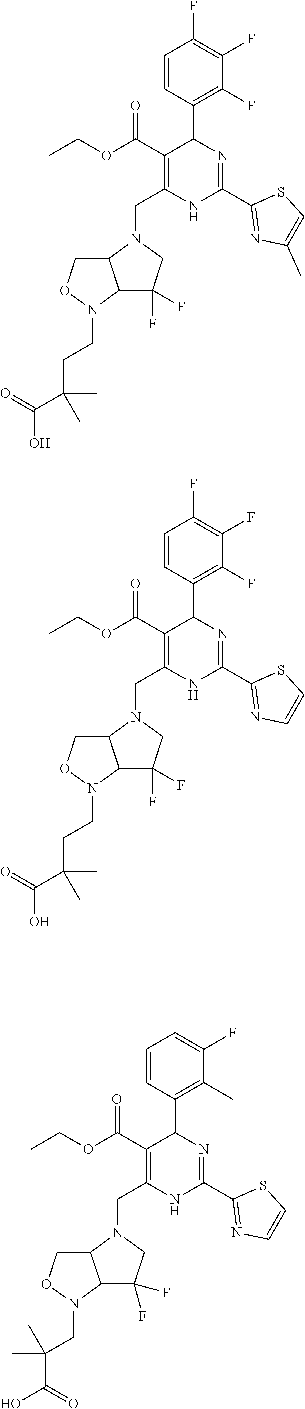 [SMILES: CCOC(=O)C1=C(CN2CC(F)(F)C3C2CON3CCC(C)(C)C(=O)O)NC(c2nc(C)cs2)=NC1c1ccc(F)c(F)c1F ; CCOC(=O)C1=C(CN2CC(F)(F)C3C2CON3CCC(C)(C)C(=O)O)NC(c2nccs2)=NC1c1ccc(F)c(F)c1F ; CCOC(=O)C1=C(CN2CC(F)(F)C3C2CON3CC(C)(C)C(=O)O)NC(c2nccs2)=NC1c1cccc(F)c1C]

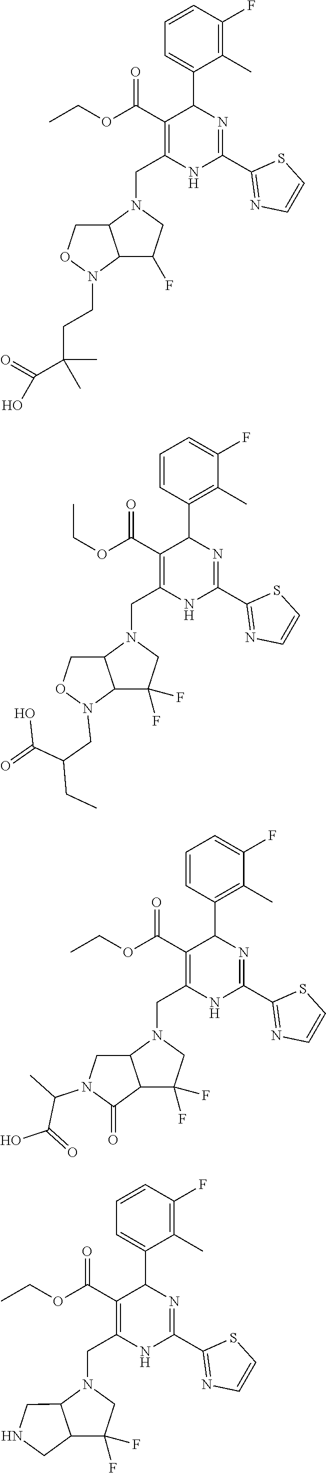 [SMILES: CCOC(=O)C1=C(CN2CC(F)C3C2CON3CCC(C)(C)C(=O)O)NC(c2nccs2)=NC1c1cccc(F)c1C ; CCOC(=O)C1=C(CN2CC(F)(F)C3C2CON3CC(CC)C(=O)O)NC(c2nccs2)=NC1c1cccc(F)c1C ; CCOC(=O)C1=C(CN2CC(F)(F)C3C(=O)N(C(C)C(=O)O)CC32)NC(c2nccs2)=NC1c1cccc(F)c1C ; CCOC(=O)C1=C(CN2CC(F)(F)C3CNCC32)NC(c2nccs2)=NC1c1cccc(F)c1C]

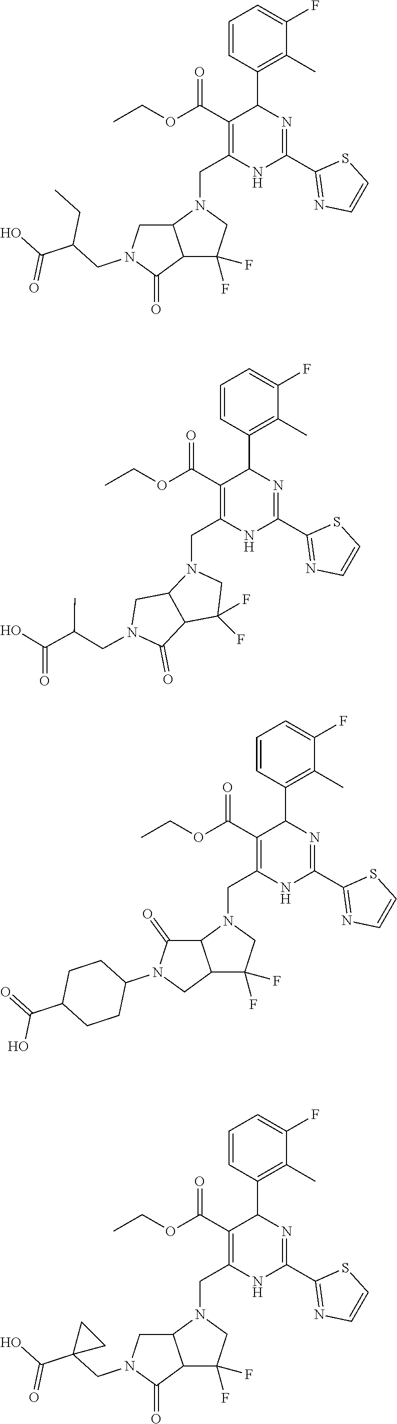 [SMILES: CCOC(=O)C1=C(CN2CC(F)(F)C3C(=O)N(CC(CC)C(=O)O)CC32)NC(c2nccs2)=NC1c1cccc(F)c1C ; CCOC(=O)C1=C(CN2CC(F)(F)C3C(=O)N(CC(C)C(=O)O)CC32)NC(c2nccs2)=NC1c1cccc(F)c1C ; CCOC(=O)C1=C(CN2CC(F)(F)C3CN(C4CCC(C(=O)O)CC4)C(=O)C32)NC(c2nccs2)=NC1c1cccc(F)c1C ; CCOC(=O)C1=C(CN2CC(F)(F)C3C(=O)N(CC4(C(=O)O)CC4)CC32)NC(c2nccs2)=NC1c1cccc(F)c1C]

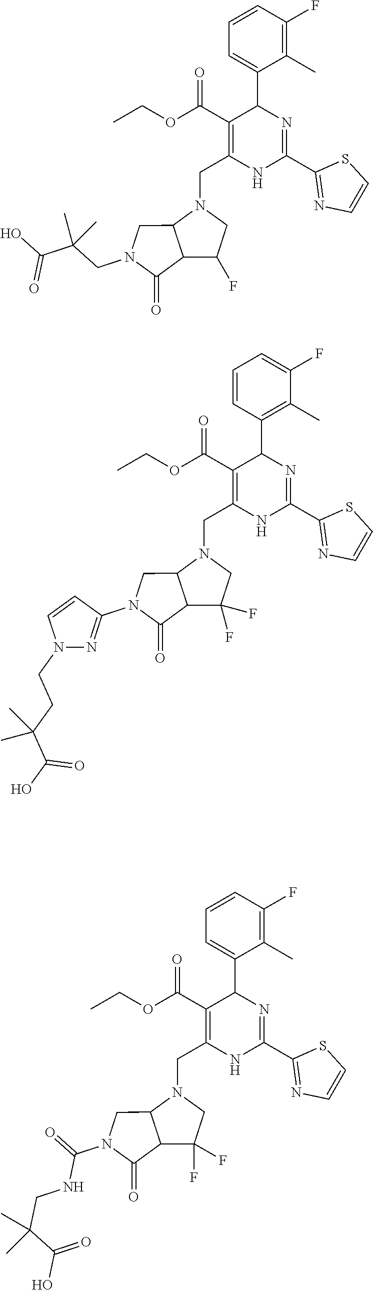 [SMILES: CCOC(=O)C1=C(CN2CC(F)C3C(=O)N(CC(C)(C)C(=O)O)CC32)NC(c2nccs2)=NC1c1cccc(F)c1C ; CCOC(=O)C1=C(CN2CC(F)(F)C3C(=O)N(c4ccn(CCC(C)(C)C(=O)O)n4)CC32)NC(c2nccs2)=NC1c1cccc(F)c1C ; CCOC(=O)C1=C(CN2CC(F)(F)C3C(=O)N(C(=O)NCC(C)(C)C(=O)O)CC32)NC(c2nccs2)=NC1c1cccc(F)c1C]

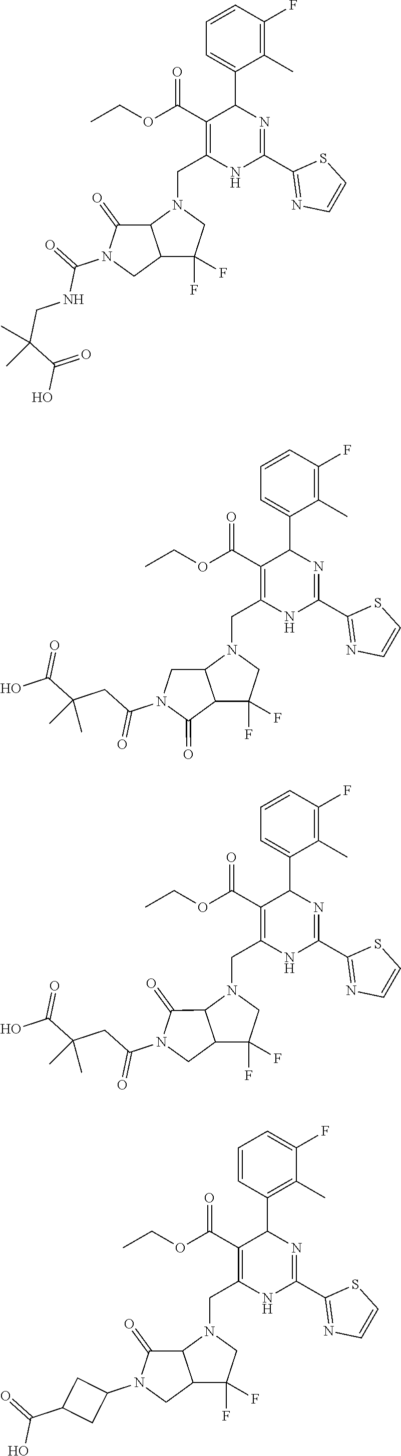 [SMILES: CCOC(=O)C1=C(CN2CC(F)(F)C3CN(C(=O)NCC(C)(C)C(=O)O)C(=O)C32)NC(c2nccs2)=NC1c1cccc(F)c1C ; CCOC(=O)C1=C(CN2CC(F)(F)C3C(=O)N(C(=O)CC(C)(C)C(=O)O)CC32)NC(c2nccs2)=NC1c1cccc(F)c1C ; CCOC(=O)C1=C(CN2CC(F)(F)C3CN(C(=O)CC(C)(C)C(=O)O)C(=O)C32)NC(c2nccs2)=NC1c1cccc(F)c1C ; CCOC(=O)C1=C(CN2CC(F)(F)C3CN(C4CC(C(=O)O)C4)C(=O)C32)NC(c2nccs2)=NC1c1cccc(F)c1C]

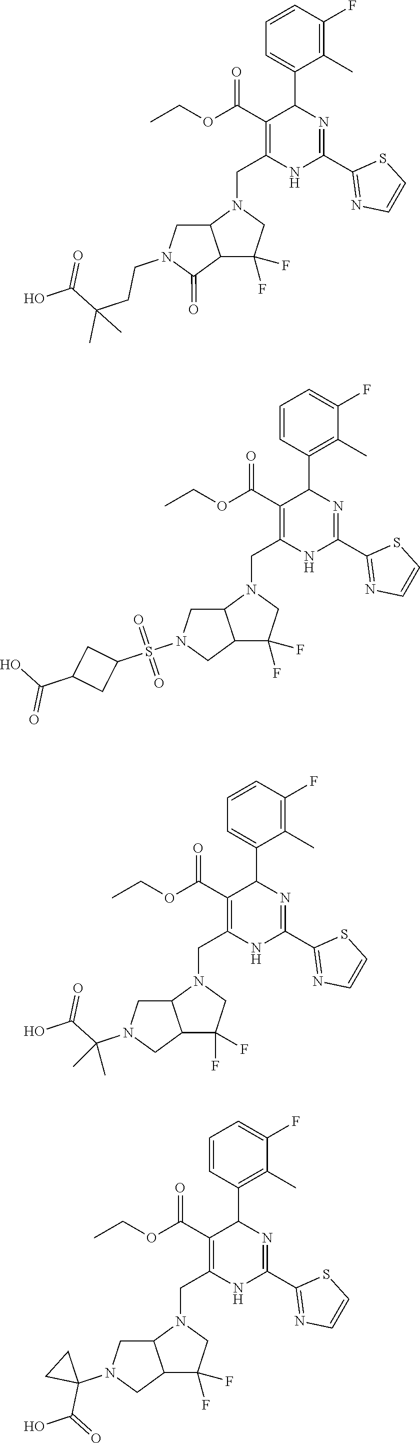 [SMILES: CCOC(=O)C1=C(CN2CC(F)(F)C3C(=O)N(CCC(C)(C)C(=O)O)CC32)NC(c2nccs2)=NC1c1cccc(F)c1C ; CCOC(=O)C1=C(CN2CC(F)(F)C3CN(S(=O)(=O)C4CC(C(=O)O)C4)CC32)NC(c2nccs2)=NC1c1cccc(F)c1C ; CCOC(=O)C1=C(CN2CC(F)(F)C3CN(C(C)(C)C(=O)O)CC32)NC(c2nccs2)=NC1c1cccc(F)c1C ; CCOC(=O)C1=C(CN2CC(F)(F)C3CN(C4(C(=O)O)CC4)CC32)NC(c2nccs2)=NC1c1cccc(F)c1C]

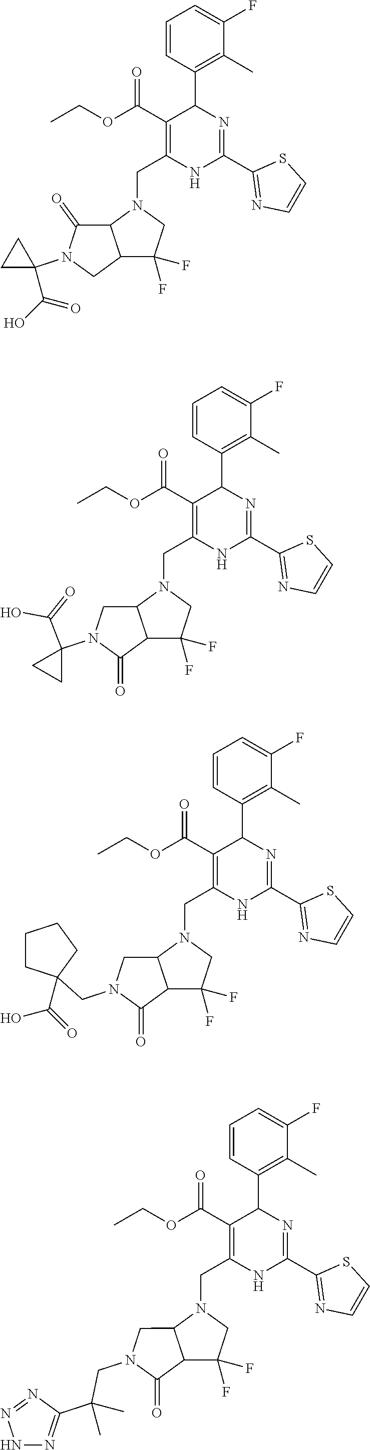 [SMILES: CCOC(=O)C1=C(CN2CC(F)(F)C3CN(C4(C(=O)O)CC4)C(=O)C32)NC(c2nccs2)=NC1c1cccc(F)c1C ; CCOC(=O)C1=C(CN2CC(F)(F)C3C(=O)N(C4(C(=O)O)CC4)CC32)NC(c2nccs2)=NC1c1cccc(F)c1C ; CCOC(=O)C1=C(CN2CC(F)(F)C3C(=O)N(CC4(C(=O)O)CCCC4)CC32)NC(c2nccs2)=NC1c1cccc(F)c1C ; CCOC(=O)C1=C(CN2CC(F)(F)C3C(=O)N(CC(C)(C)c4nn[nH]n4)CC32)NC(c2nccs2)=NC1c1cccc(F)c1C]

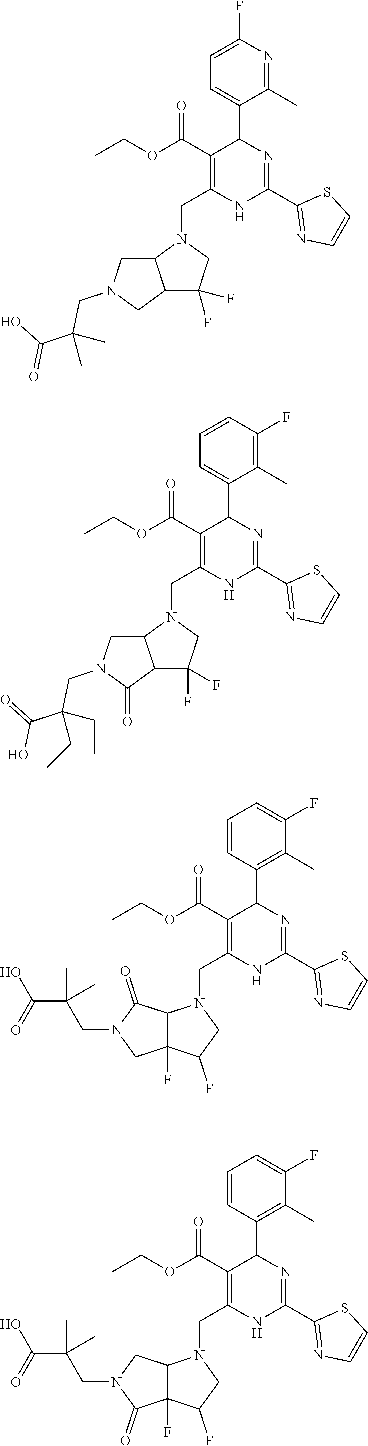 [SMILES: CCOC(=O)C1=C(CN2CC(F)(F)C3CN(CC(C)(C)C(=O)O)CC32)NC(c2nccs2)=NC1c1ccc(F)nc1C ; CCOC(=O)C1=C(CN2CC(F)(F)C3C(=O)N(CC(CC)(CC)C(=O)O)CC32)NC(c2nccs2)=NC1c1cccc(F)c1C ; CCOC(=O)C1=C(CN2CC(F)C3(F)CN(CC(C)(C)C(=O)O)C(=O)C23)NC(c2nccs2)=NC1c1cccc(F)c1C ; CCOC(=O)C1=C(CN2CC(F)C3(F)C(=O)N(CC(C)(C)C(=O)O)CC23)NC(c2nccs2)=NC1c1cccc(F)c1C]

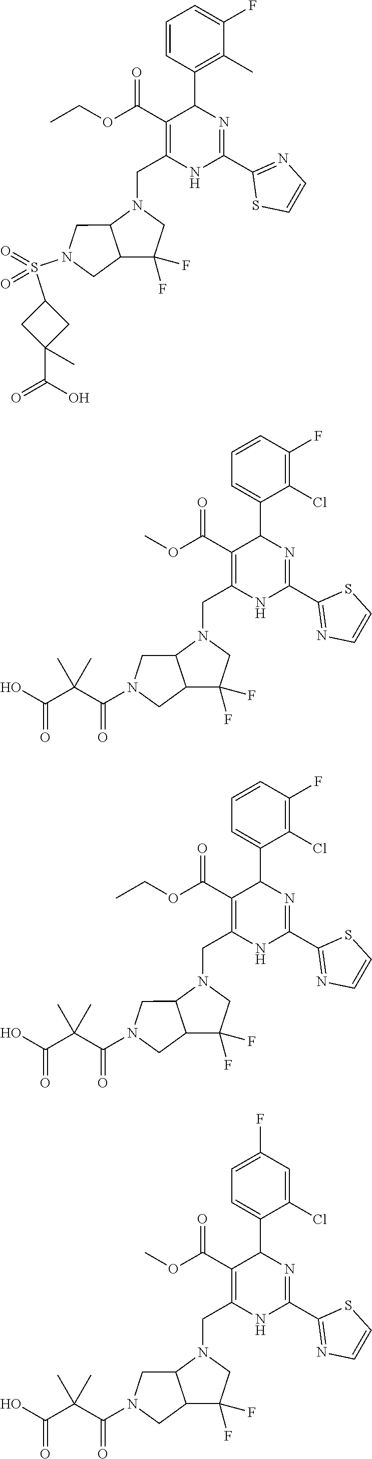 [SMILES: CCOC(=O)C1=C(CN2CC(F)(F)C3CN(S(=O)(=O)C4CC(C)(C(=O)O)C4)CC32)NC(c2nccs2)=NC1c1cccc(F)c1C ; COC(=O)C1=C(CN2CC(F)(F)C3CN(C(=O)C(C)(C)C(=O)O)CC32)NC(c2nccs2)=NC1c1cccc(F)c1Cl ; CCOC(=O)C1=C(CN2CC(F)(F)C3CN(C(=O)C(C)(C)C(=O)O)CC32)NC(c2nccs2)=NC1c1cccc(F)c1Cl ; COC(=O)C1=C(CN2CC(F)(F)C3CN(C(=O)C(C)(C)C(=O)O)CC32)NC(c2nccs2)=NC1c1ccc(F)cc1Cl]

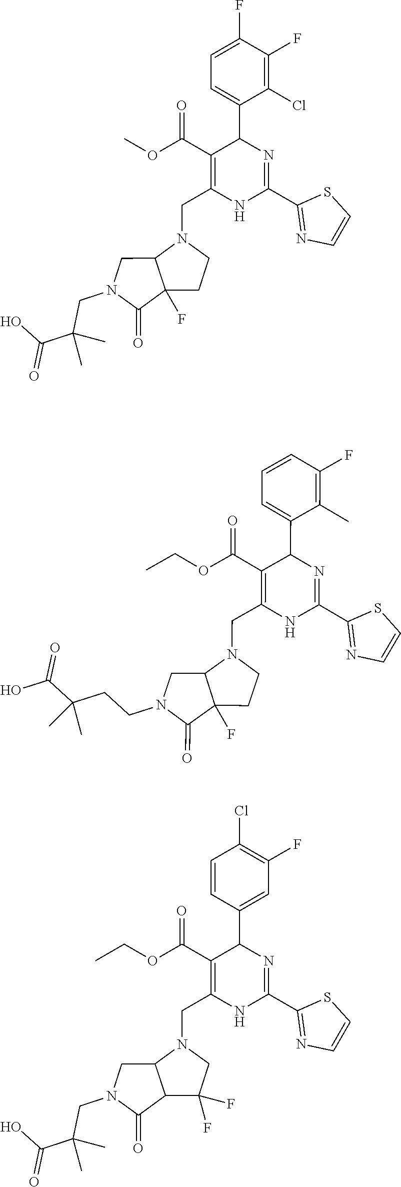 [SMILES: COC(=O)C1=C(CN2CCC3(F)C(=O)N(CC(C)(C)C(=O)O)CC23)NC(c2nccs2)=NC1c1ccc(F)c(F)c1Cl ; CCOC(=O)C1=C(CN2CCC3(F)C(=O)N(CCC(C)(C)C(=O)O)CC23)NC(c2nccs2)=NC1c1cccc(F)c1C ; CCOC(=O)C1=C(CN2CC(F)(F)C3C(=O)N(CC(C)(C)C(=O)O)CC32)NC(c2nccs2)=NC1c1ccc(Cl)c(F)c1]

Preferred compounds according to the invention are compounds or a stereoisomer or tautomeric form thereof with a formula as represented in the synthesis of compounds section and Table 1, and of which the activity is displayed in Table 3.

The disclosed compounds may possess one or more stereocenters, and each stereocenter may exist independently in either the R or S configuration. The stereochemical configuration may be assigned at indicated centers as (*) when the absolute stereochemistry is undetermined at the stereocenter although the compound itself has been isolated as a single stereoisomer and is enatiomerically/diastereomerically pure.

In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein.

Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the disclosed compound described herein. In another embodiment, compounds described herein contain one or more chiral centers. These compounds are prepared by any means, including stereoselective synthesis, enantioselective synthesis or separation of a mixture of enantiomers or diastereomers. Resolution of compounds and isomers thereof is achieved by any means including, by way of non-limiting example, chemical processes, enzymatic processes, fractional crystallization, distillation, and chromatography.

When the absolute R or S stereochemistry of a compound cannot be determined, it can be identified by the retention time after chromatography under particular chromatographic conditions as determined by chromatography column, eluent, etc.

For some compounds, the stereochemical configuration at indicated centres has been assigned as “R*”, “S*” when the absolute stereochemistry is undetermined although the compound itself has been isolated as a single stereoisomer and is enantiomerically/diastereomerically pure.

In one embodiment, the disclosed compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature.

Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements).

In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and techniques known to a person skilled in the art. General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein. General synthesis schemes are given in the Examples below.

Accordingly, a process is provided for producing the compound of Formula (I), wherein said process comprises reacting a compound of Formula (I-2)

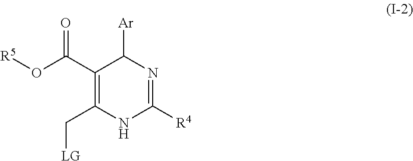

wherein Ar, R⁴-R⁵ are as defined in Formula (I), and LG represents a suitable leaving group, such as for example, bromo; with a compound of Formula (V)

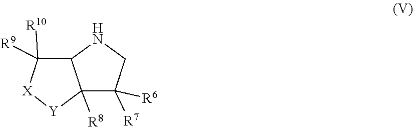

wherein R⁶-R¹⁰, X and Y are as defined in Formula (I);

under suitable nucleophilic substitution conditions, for example, in the presence of a suitable base, such as for example triethanolamine.

Methods and Uses

Provided herein is a method of treating an HBV infection in an individual in need thereof, comprising administering to the individual a therapeutically effective amount of a disclosed compound.

Also provided herein is a method of eradicating an HBV infection in an individual in need thereof, comprising administering to the individual a therapeutically effective amount of a disclosed compound.

Provided herein is a method of reducing viral load associated with an HBV infection in an individual in need thereof, comprising administering to the individual a therapeutically effective amount of a disclosed compound.

Further, provided herein is a method of reducing reoccurrence of an HBV infection in an individual in need thereof, comprising administering to the individual a therapeutically effective amount of a disclosed compound.

Provided herein is a method of inhibiting or reducing the formation or presence of HBV DNA-containing particles or HBV RNA-containing particles in an individual in need thereof, comprising administering to the individual a therapeutically effective amount of a disclosed compound.

Where the invention is said to relate to a method of treating an individual, it is understood that such method is to be interpreted in certain jurisdictions as a medical use, e.g. a compound or a composition according to the invention for use in treating an individual; or a use of the compound or the composition according to the invention, for the manufacture of a medicament, in particular for treating an individual. Therefore, for example, the invention also relates to a compound or a pharmaceutical composition as disclosed herein for use in the prevention or treatment of an HBV infection. Also provided herein, is a compound or a pharmaceutical composition as disclosed herein for use in the reduction of viral load associated with an HBV infection. Further provided herein, is a compound or a pharmaceutical composition as disclosed herein for use in the reduction of reoccurrence of an HBV infection in an individual. Also provided herein, is a compound or a pharmaceutical composition as disclosed herein, for use in the inhibition or reduction of the formation or presence of HBV DNA-containing particles or HBV RNA-containing particles in an individual.

In certain aspects, the methods, uses and/or compositions described herein are effective for inhibiting or reducing the formation or presence of HBV-associated particles in vitro or in vivo (e.g., in a cell, in a tissue, in an organ (e.g., in the liver), in an organism or the like). HBV-associated particles may contain HBV DNA (i.e., linear and/or covalently closed circular DNA (cccDNA)) and/or HBV RNA (i.e., pre-genomic RNA and/or sub-genomic RNA). Accordingly, HBV-associated particles include HBV DNA-containing particles or HBV RNA-containing particles.

As used herein, “HBV-associated particles” refer to both infectious HBV virions (i.e., Dane particles) and non-infectious HBV subviral particles (i.e., HBV filaments and/or HBV spheres). HBV virions comprise an outer envelope including surface proteins, a nucleocapsid comprising core proteins, at least one polymerase protein, and an HBV genome. HBV filaments and HBV spheres comprise HBV surface proteins, but lack core proteins, polymerase and an HBV genome. HBV filaments and HBV spheres are also known collectively as surface antigen (HBsAg) particles. HBV spheres comprise middle and small HBV surface proteins. HBV filaments also include middle, small and large HBV surface proteins.

HBV subviral particles can include the nonparticulate or secretory HBeAg, which serves as a marker for active replication of HBV.

Provided herein is a method of reducing an adverse physiological impact of an HBV infection in an individual in need thereof, comprising administering to the individual a therapeutically effective amount of a disclosed compound.

Also provided herein is a method of reducing, slowing, or inhibiting an HBV infection in an individual in need thereof, comprising administering to the individual a therapeutically effective amount of a disclosed compound.

Provided herein is a method of inducing reversal of hepatic injury from an HBV infection in an individual in need thereof, comprising administering to the individual a therapeutically effective amount of a disclosed compound.

Provided herein is a method of reducing the physiological impact of long-term antiviral therapy for HBV infection in an individual in need thereof, comprising administering to the individual a therapeutically effective amount of a disclosed compound.

Provided herein is a method of prophylactically treating an HBV infection in an individual in need thereof, wherein the individual is afflicted with a latent HBV infection, comprising administering to the individual a therapeutically effective amount of a disclosed compound.

Also provided herein, is a compound or a pharmaceutical composition as disclosed herein, for use in the reduction of an adverse physiological impact of an HBV infection in an individual.

Also provided herein is a compound or a pharmaceutical composition as disclosed herein, for use in the reduction, slowing or inhibition of an HBV infection in an individual. Also provided herein, is a compound or a pharmaceutical composition as disclosed herein for use in inducing reversal of hepatic injury from an HBV infection in an individual.

Also provided herein is a compound or a pharmaceutical composition as disclosed herein for use in reducing the physiological impact of long-term antiviral therapy for HBV infection in an individual. Further provided herein is a compound or a pharmaceutical composition as disclosed herein for use in the prophylactic treatment of an HBV infection in an individual, wherein the individual is afflicted with a latent HBV infection.

In one embodiment, the individual is refractory to other therapeutic classes of HBV drugs (e.g, HBV polymerase inhibitors, interferons, viral entry inhibitors, viral maturation inhibitors, literature-described capsid assembly modulators, antiviral compounds of distinct or unknown mechanism, and the like, or combinations thereof). In another embodiment, the disclosed method or use reduces viral load in an individual suffering from an HBV infection to a greater extent or at a faster rate compared to the extent that other therapeutic classes of HBV drugs reduce viral load in the individual.

In one embodiment, the administering of a disclosed compound, or a pharmaceutically acceptable salt thereof, allows for administering of the at least one additional therapeutic agent at a lower dose or frequency as compared to the administering of the at least one additional therapeutic agent alone that is required to achieve similar results in prophylactically treating an HBV infection in an individual in need thereof.

In one embodiment, the administering of a disclosed compound, or a pharmaceutically acceptable salt thereof, reduces the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In one embodiment, the disclosed method or use reduces viral load in an individual suffering from an HBV infection, thus allowing lower doses or varying regimens of combination therapies to be used.

In one embodiment, the disclosed method or use causes a lower incidence of viral mutation or viral resistance compared to other classes of HBV drugs, thereby allowing for long term therapy and minimizing the need for changes in treatment regimens.

In one embodiment, the administering of a compound the invention, or a pharmaceutically acceptable salt thereof, causes a lower incidence of viral mutation or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In one embodiment, the disclosed method or use increases the seroconversion rate from HBV infected to non-HBV infected or from detectable HBV viral load to non-detectable HBV viral load beyond that of current treatment regimens. As used herein, “seroconversion” refers to the period of time during which HBV antibodies develop and become detectable.

In one embodiment, the disclosed method or use increases or normalizes or restores normal health, elicits full recovery of normal health, restores life expectancy, or resolves the viral infection in the individual in need thereof.

In one embodiment, the disclosed method or use eliminates or decreases the number of HBV RNA particles that are released from HBV infected cells thus enhancing, prolonging, or increasing the therapeutic benefit of the disclosed compounds.

In one embodiment, the disclosed method or use eradicates HBV from an individual infected with HBV, thereby obviating the need for long term or life-long treatment, or shortening the duration of treatment, or allowing for reduction in dosing of other antiviral agents.

In another embodiment, the disclosed method or use further comprises monitoring or detecting the HBV viral load of the subject, and wherein the method is carried out for a period of time including until such time that the HBV virus is undetectable.

Accordingly, in one embodiment, provided herein is a method of treating an HBV infection in an individual in need thereof, comprising administering to the individual a therapeutically effective amount of a compound of Formula (I), or a pharmaceutically acceptable salt thereof.

Accordingly, in one embodiment, provided herein is a method of treating an HBV infection in an individual in need thereof, comprising administering to the individual a therapeutically effective amount of a compound of Formula (I), or a pharmaceutically acceptable salt thereof. In another embodiment, provided herein is a method of treating an HBV infection in an individual in need thereof, comprising administering to the individual a therapeutically effective amount of a compound according to the invention, e.g. those of Table 1, or a pharmaceutically acceptable salt thereof.

In an embodiment of any of the methods provided herein, the method or use can further comprise monitoring the HBV viral load of the subject, wherein the method is carried out for a period of time such that the HBV virus is undetectable.

Combination Therapies

The disclosed compounds may be useful in combination with one or more additional compounds useful for treating HBV infection. These additional compounds may comprise other disclosed compounds and/or compounds known to treat, prevent, or reduce the symptoms or effects of HBV infection. Such compounds include, but are not limited to, HBV polymerase inhibitors, interferons, viral entry inhibitors, viral maturation inhibitors, literature-described capsid assembly modulators, reverse transcriptase inhibitors, immunomodulatory agents, TLR-agonists, and other agents with distinct or unknown mechanisms that affect the HBV life cycle or affect the consequences of HBV infection, e.g. the additional compounds may comprise HBV combination drugs, HBV vaccines, HBV DNA polymerase inhibitors, immunomodulators, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other HBV drugs.

In non-limiting examples, the disclosed compounds may be used in combination with one or more drugs (or a salt thereof) selected from the group comprising:

HBV reverse transcriptase inhibitors, and DNA and RNA polymerase inhibitors.

In one embodiment, the additional therapeutic agent is an interferon. The term “interferon” or “IFN” refers to any member of the family of highly homologous species-specific proteins that inhibit viral replication and cellular proliferation and modulate immune response. Human interferons are grouped into three classes: Type I, Type II, and Type III. Recombinant forms of interferons that have been developed and are commercially available are encompassed by the term “interferon” as used herein. Subtypes of interferons, such as chemically modified or mutated interferons, are also encompassed by the term “interferon” as used herein.

Accordingly, in one embodiment, the compounds of Formula (I) can be administered in combination with an interferon.

In another embodiment, the additional therapeutic agent is selected from immune modulator or immune stimulator therapies, which includes biological agents belonging to the interferon class.

Further, the additional therapeutic agent may be an agent of distinct or unknown mechanism including agents that disrupt the function of other essential viral protein(s) or host proteins required for HBV replication or persistence.

In another embodiment, the additional therapeutic agent is an antiviral agent that blocks viral entry or maturation or targets the HBV polymerase such as nucleoside or nucleotide or non-nucleos(t)ide polymerase inhibitors.

In an embodiment, the additional therapeutic agent is an immunomodulatory agent that induces a natural, limited immune response leading to induction of immune responses against unrelated viruses. In other words, the immunomodulatory agent can effect maturation of antigen presenting cells, proliferation of T-cells and cytokine release (e.g., IL-12, IL-18, IFN-alpha, -beta, and -gamma and TNF-alpha among others).

In a further embodiment, the additional therapeutic agent is a TLR modulator or a TLR agonist, such as a TLR-7 agonist or TLR-9 agonist.

In any of the methods provided herein, the method may further comprise administering to the individual at least one HBV vaccine, a nucleoside HBV inhibitor, an interferon or any combination thereof.

In one embodiment, the methods described herein further comprise administering at least one additional therapeutic agent selected from the group consisting of nucleotide/nucleoside analogs, entry inhibitors, fusion inhibitors, and any combination of these or other antiviral mechanisms.

In another aspect, provided herein is method of treating an HBV infection in an individual in need thereof, comprising reducing the HBV viral load by administering to the individual a therapeutically effective amount of a disclosed compound alone or in combination with a reverse transcriptase inhibitor; and further administering to the individual a therapeutically effective amount of HBV vaccine.

In another aspect, provided herein is a method of treating an HBV infection in an individual in need thereof, comprising reducing the HBV viral load by administering to the individual a therapeutically effective amount of a disclosed compound alone or in combination with a antisense oligonucleotide or RNA interference agent that targets HBV nucleic acids; and further administering to the individual a therapeutically effective amount of HBV vaccine. The antisense oligonucleotide or RNA interference agent possesses sufficient complementarity to the target HBV nucleic acids to inhibit replication of the viral genome, transcription of viral RNAs, or translation of viral proteins.

In another embodiment, the disclosed compound and the at least one additional therapeutic agent are co-formulated. In yet another embodiment, the disclosed compound and the at least one additional therapeutic agent are co-administered.

For any combination therapy described herein, synergistic effect may be calculated, for example, using suitable methods such as the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In an embodiment of any of the methods of administering combination therapies provided herein, the method can further comprise monitoring or detecting the HBV viral load of the subject, wherein the method is carried out for a period of time including until such time that the HBV virus is undetectable.

Administration/Dosage/Formulations

In another aspect, provided herein is a pharmaceutical composition comprising at least one disclosed compound, or a pharmaceutically acceptable salt thereof, together with a pharmaceutically acceptable carrier.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the invention are dictated by and directly dependent on (a) the unique characteristics of the disclosed compound and the particular therapeutic effect to be achieved, and (b) the limitations inherent in the art of compounding/formulating such a disclosed compound for the treatment of HBV infection in a patient.

In one embodiment, the compositions of the invention are formulated using one or more pharmaceutically acceptable excipients or carriers. In one embodiment, the pharmaceutical compositions of the invention comprise a therapeutically effective amount of a disclosed compound and a pharmaceutically acceptable carrier. Thus, illustrating the invention is a process for preparing a pharmaceutical composition, comprising mixing at least one pharmaceutically acceptable carrier with a therapeutically effective amount of a disclosed compound.

In some embodiments, the dose of a disclosed compound is from about 1 mg to about 2,500 mg. Similarly, in some embodiments, a dose of a second compound (i.e., another drug for HBV treatment) as described herein is less than about 1,000 mg.

In one embodiment, the present invention is directed to a packaged pharmaceutical composition comprising a container holding a therapeutically effective amount of a disclosed compound, alone or in combination with a second pharmaceutical agent; and instructions for using the compound to treat, prevent, or reduce one or more symptoms of HBV infection in a patient.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

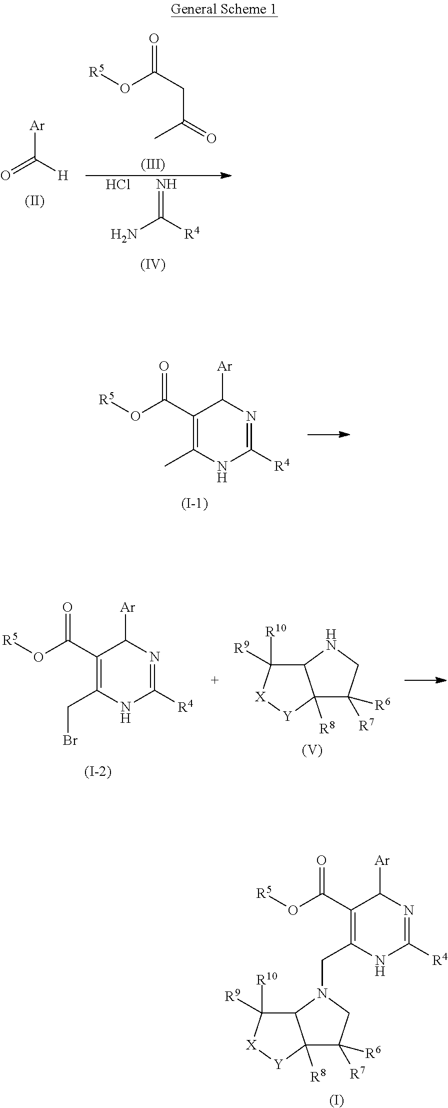

The preparation of compound I is shown in the above Scheme 1:

Compound I-1 can be prepared by the condensation of aldehyde II, acetoacetate III and amidine IV in the presence of a base such as NaOAc. Compound I-2 was prepared from compound I-1 using brominating reagent such as N-Bromosuccinimide. Coupling of compound I-2 and compound V in the presence of a base such as triethanolamine affords compound I. In Scheme 1, all variables are as defined in Formula (I).

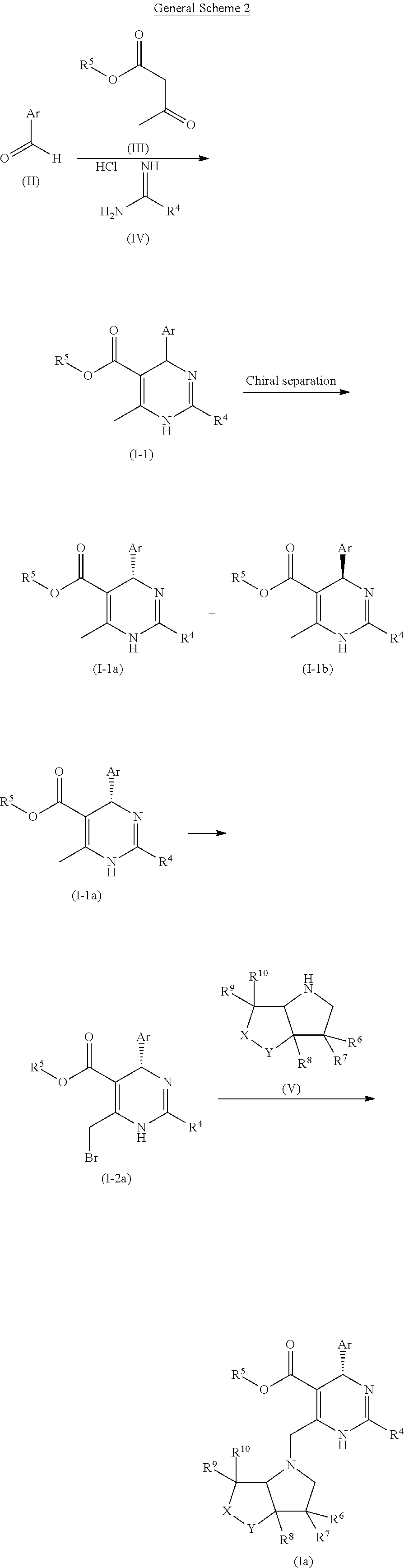

A chiral separation can be performed during the synthetic process, as indicated in General Scheme 2. For example, compound (I-la) can be prepared by the condensation of aldehyde (II), acetoacetate (III) and amidine (IV) in the presence of a base, such as NaOAc, in a suitable solvent, such as for example ethanol, under suitable reaction conditions, such as for example at a temperature of about 70-100° C. under an inert atmosphere, e.g. nitrogen, for a sufficient period of time, typically from 6-12 hours. Compound (I-1) can be subjected to chiral separation to provide compound (I-la) and compound (I-1b).

Compound (I-2a) can be prepared from compound (I-la) using a brominating reagent, such as for example, N-bromosuccinimide, in a suitable solvent, such as carbon tetrachloride, under suitable reaction conditions, such as for example at a temperature of about room temperature to about 60° C. under an inert atmosphere, e.g. nitrogen, for a sufficient period of time, typically 1 hour. Compound (Ia) can be prepared by coupling compounds (I-2a) and (V) in the presence of a base, such as for example triethanolamine, in a suitable solvent, such as for example dichloromethane, under suitable reaction conditions, such as for example at a temperature of about 40° C. under an inert atmosphere, e.g. nitrogen, for a sufficient period of time, typically about 2 hours, followed by treatment under acidic conditions, such as for example aqueous hydrochloric acid at 0° C.

Chemistry

Several methods for preparing the compounds of this invention are illustrated hereinbelow. Unless otherwise noted, all starting materials were obtained from commercial suppliers and used without further purification.

Hereinafter, ACN means acetonitrile, AcOH means acetic acid, Boc means tert-butyloxycarbonyl, Bn means benzyl, calcd. means calculated, Cbz means benzyloxycarbonyl, col. means column, conc. means concentrated, m-CPBA means 3-chloroperbenzoic acid, DAST means (diethylamino)sulfur trifluoride, DCM means dichloromethane, DEA means diethanolamine, DIEA means N,N-diisopropylethyl amine, DMAP means 4-(dimethylamino)pyridine, DMF means dimethylformamide, DMP means Dess-Martin periodinane, EA means ethyl acetate, ee means enantiomeric excess, ESI means electrospray ionization, HATU means 2-(7-azabenzotriazol-1-yl)-N,N,N′,N′-tetramethyluronium hexafluorophosphate, Hex means hexane, HNMR means ¹H NMR, HPLC means high performance liquid chromatography, IPA means isopropyl alcohol, LC-MS or LCMS means liquid chromatography-mass spectrometry, LDA means lithium diisopropylamide, Ms means methanesulfonyl, PE means petroleum ether, PMB means 4-methoxybenzyl, prep. means preparative, Prep-HPLC means preparative HPLC, R_{T} or Rt mean retention time, (s) or (s) mean solid, sat. means saturated, TBAF means tetrabutylammonium fluoride, TBS means tert-butyldimethylsilyl, TEA means triethylamine, THE means tetrahydrofuran, T or Temp mean temperature, TsCl means 4-toluenesulfonyl chloride, t-BuOK means potassium tert-butoxide, W means wavelength.

### Preparation of ethyl 4-(2-chloro-3-fluorophenyl)-6-methyl-2-(thiazol-2-yl)-1,4-dihydropyrimidine-5-carboxylate (H1)

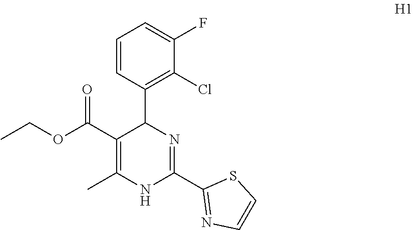 [SMILES: CCOC(=O)C1=C(C)NC(c2nccs2)=NC1c1cccc(F)c1Cl]

To a solution of 2-chloro-3-fluorobenzaldehyde (8.8 g, 55.7 mmol), ethyl 3-oxobutanoate (7.24 g, 55.7 mmol) in isopropanol (40 mL) was added piperidine (473 mg, 5.57 mmol) and AcOH (334 mg, 5.57 mmol). After stirred at room temperature for 4 hours, the mixture was added thiazole-2-carboximidamide (6.4 g, 39 mmol) and triethylamine (5.62 g, 55.7 mmol) at room temperature over 15 minutes. The reaction mixture was stirred at 75° C. for 12 hours. It was cooled to room temperature, extracted with ethyl acetate, washed with brine, dried over Na₂SO₄ and purified by silica gel column chromatography (petroleum ether:ethyl acetate=20:1) to give the title compound H1 (5.45 g, 95% purity from 1H NMR, 26% yield) as yellow solids. LC-MS (ESI): mass calcd. for C₁₇H₁₅ClFN₃O₂S 379.1, m/z found 380.1 [M+H]⁺. ¹H NMR (400 MHz, CDCl₃) δ 7.84-7.80 (m, 1.7H), 7.50 (d, J=3.6 Hz, 0.3H), 7.47 (s, 0.3H), 7.44 (d, J=3.2 Hz, 0.7H), 7.23-7.14 (m, 2H), 7.09-7.01 (m, 1H), 6.27 (s, 0.7H), 6.14 (d, J=2.4 Hz, 0.3H), 4.13-3.98 (m, 2H), 2.57 (s, 0.7H), 2.52 (s, 2.3H), 1.13-1.10 (m, 3H).

### Chiral Separation of ethyl 4-(2-chloro-3-fluorophenyl)-6-methyl-2-(thiazol-2-yl)-1,4-dihydropyrimidine-5-carboxylate (H1)

The racemic mixture ethyl 4-(2-chloro-3-fluorophenyl)-6-methyl-2-(thiazol-2-yl)-1,4-dihydropyrimidine-5-carboxylate H1 (5.45 g, 13.7 mmol) was separated by chiral separation (separation condition: column: Chiralpak IC 5 μm 20*250 mm; Mobile Phase: Hex:EtOH:DEA=95:5:0.3 at 28 mL/min, Temp: 30° C., Wavelength: 254 nm) to give H1-A (2.5 g, 90% purity from ¹HNMR, 46% yield, 100% ee) and H1-B (2.48 g, 90% purity from ¹HNMR, 46% yield, 92.1% ee) as yellow solids.

H1-A: LC-MS (ESI): mass calcd. for C₁₇H₁₅ClFN₃O₂S 379.06, m/z found 380.1 [M+H]⁺. Chiral analysis (Column: Chiralpak IA 5 μm 4.6*250 mm; Mobile Phase: Hex:EtOH:DEA=90:10:0.2 at 1.0 mL/min; Temp: 30° C.; Wavelength: 254 nm, R_{T}=7.438 min). ¹H NMR (400 MHz, CDCl₃) δ 7.84-7.80 (m, 1.7H), 7.51-7.44 (m, 1.3H), 7.22-7.14 (m, 2H), 7.09-7.01 (m, 1H), 6.27 (s, 0.7H), 6.14 (s, 0.3H), 4.05-4.00 (m, 2H), 2.57 (s, 0.7H), 2.52 (s, 2.3H), 1.13-1.10 (m, 3H).

H1-B: LC-MS (ESI): mass calcd. for C₁₇H₁₅ClFN₃O₂S 379.06, m/z found 380.1 [M+H]⁺. Chiral analysis (Column: Chiralpak IA 5 μm 4.6*250 mm; Mobile Phase: Hex:EtOH:DEA=90:10:0.2 at 1.0 mL/min; Temp: 30° C.; Wavelength: 254 nm, R_{T}=6.903 min). ¹H NMR (400 MHz, CDCl₃) δ 7.84-7.80 (m, 1.7H), 7.51-7.43 (m, 1.3H), 7.22-7.14 (m, 2H), 7.09-7.01 (m, 1H), 6.27 (s, 0.7H), 6.14 (s, 0.3H), 4.10-3.98 (m, 2H), 2.57 (s, 0.7H), 2.51 (s, 2.3H), 1.13-1.10 (m, 3H).

### Preparation of ethyl 6-(bromomethyl)-4-(2-chloro-3-fluorophenyl)-2-(thiazol-2-yl)-1,4-dihydropyrimidine-5-carboxylate (H1-1A) (Single Enantiomer)

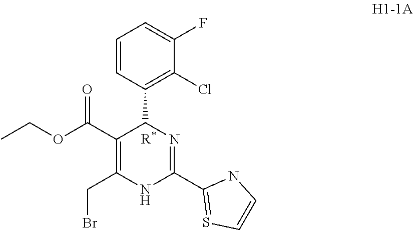

To a solution of ethyl 4-(2-chloro-3-fluorophenyl)-6-methyl-2-(thiazol-2-yl)-1,4-dihydropyrimidine-5-carboxylate H1-A (300 mg, 90% purity, 0.711 mmol) in carbon tetrachloride (5 mL) was added N-bromosuccinimide (120 mg, 0.674 mmol). After stirred at 60° C. for 1 hour, the reaction mixture was concentrated to give a residue, which was purified by silica gel column chromatography (petroleum ether:ethyl acetate=20:1 to 10:1) to give the title compound (H1-1A) (240 mg, 90% purity from HNMR, 66% yield) as yellow solids. LC-MS (ESI): mass calcd. for C₁₇H₁₄BrClFN₃O₂S 456.9, m/z found 457.9 [M+H]⁺. ¹H NMR (400 MHz, CDCl₃) δ 8.26 (s, 0.3H), 7.84 (d, J=2.8 Hz, 1H), 7.53-7.46 (in, 1.7H), 7.24-7.14 (m, 2H), 7.09-7.01 (m, 1H), 6.26 (s, 0.3H), 6.17 (s, 0.7H), 4.92 (d, J=8.0 Hz, 1H), 4.76 (d, J=11.2 Hz, 0.3H), 4.60 (d, J=8.0 Hz, 0.7H), 4.12 (q, J=7.2 Hz, 2H), 1.14 (t, J=11.2 Hz, 3H).

Using the same procedure, the following intermediates were prepared.

| | | | | Brominated |
|---|---|---|---|---|
| Aldehyde | Ketoester | Amidine | Intermediate | Intermediate |
| 2-methyl-3-fluorobenz- aldehyde | Ethyl 3- oxobutanoate | thiazole-2- carboximidamide | 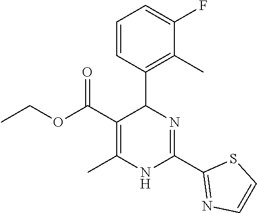 [SMILES: CCOC(=O)C1=C(C)NC(c2nccs2)=NC1c1cccc(F)c1C] H2 | 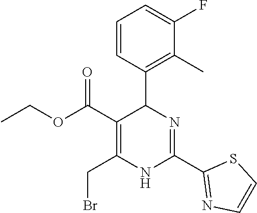 [SMILES: CCOC(=O)C1=C(CBr)NC(c2nccs2)=NC1c1cccc(F)c1C] H2-1(Racemic) H2-1A (S- enantiomer) |
| 2-chloro-4-fluorobenz- aldehyde | Methyl 3- oxobutanoate | thiazole-2- carboximidamide | 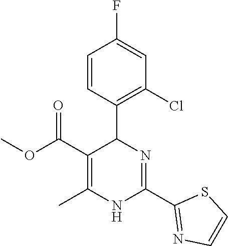 [SMILES: COC(=O)C1=C(C)NC(c2nccs2)=NC1c1ccc(F)cc1Cl] H3 | 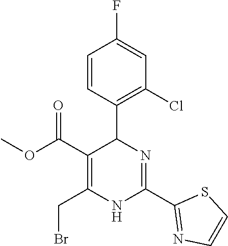 [SMILES: COC(=O)C1=C(CBr)NC(c2nccs2)=NC1c1ccc(F)cc1Cl] H3-1(Racemic) H3-1A (single enantiomer) |
| 2-methyl-3-fluorobenz- aldehyde | Methyl 3- oxobutanoate | thiazole-2- carboximidamide | 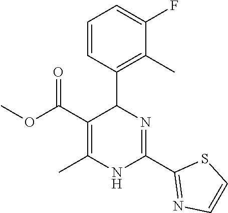 [SMILES: COC(=O)C1=C(C)NC(c2nccs2)=NC1c1cccc(F)c1C] H4 | 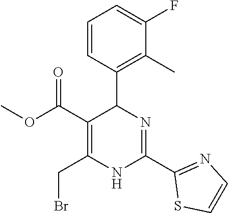 [SMILES: COC(=O)C1=C(CBr)NC(c2nccs2)=NC1c1cccc(F)c1C] H4-1B (single enantiomer) |
| 2-chloro-3,4- difluorobenz- aldehyde | Methyl 3- oxobutanoate | thiazole-2- carboximidamide | 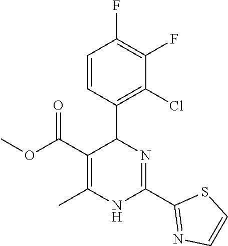 [SMILES: COC(=O)C1=C(C)NC(c2nccs2)=NC1c1ccc(F)c(F)c1Cl] H5 | 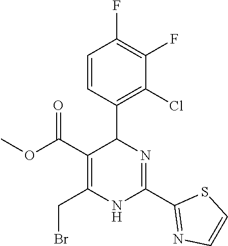 [SMILES: COC(=O)C1=C(CBr)NC(c2nccs2)=NC1c1ccc(F)c(F)c1Cl] H5-1A (single enantiomer) |
| 3,4-difluoro-2- methylbenz- aldehyde | Methyl 3- oxobutanoate | thiazole-2- carboximidamide | 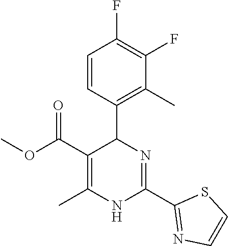 [SMILES: COC(=O)C1=C(C)NC(c2nccs2)=NC1c1ccc(F)c(F)c1C] H6 | 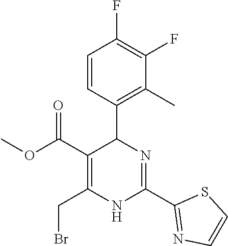 [SMILES: COC(=O)C1=C(CBr)NC(c2nccs2)=NC1c1ccc(F)c(F)c1C] H6-1B (single enantiomer) |
| 2-chloro-3,4- difluorobenz- aldehyde | Ethyl 3- oxobutanoate | thiazole-2- carboximidamide | 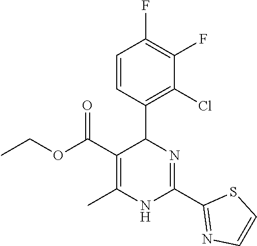 [SMILES: CCOC(=O)C1=C(C)NC(c2nccs2)=NC1c1ccc(F)c(F)c1Cl] H8 | 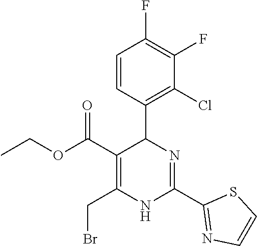 [SMILES: CCOC(=O)C1=C(CBr)NC(c2nccs2)=NC1c1ccc(F)c(F)c1Cl] H8-1 (racemic) H8-1A (single enantiomer) |
| 3,4-difluoro-2- methylbenz- aldehyde | Ethyl 3- oxobutanoate | thiazole-2- carboximidamide | 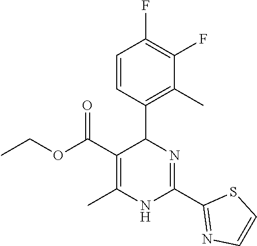 [SMILES: CCOC(=O)C1=C(C)NC(c2nccs2)=NC1c1ccc(F)c(F)c1C] H9 | 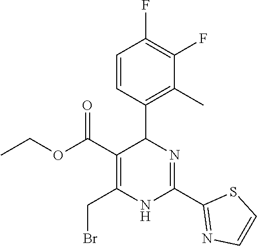 [SMILES: CCOC(=O)C1=C(CBr)NC(c2nccs2)=NC1c1ccc(F)c(F)c1C] H9-1A (single enantiomer) |
| 2-chloro-3- fluorobenzaldehyde | Methyl 3- oxobutanoate | thiazole-2- carboximidamide | 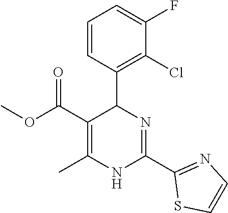 [SMILES: COC(=O)C1=C(C)NC(c2nccs2)=NC1c1cccc(F)c1Cl] H11 | 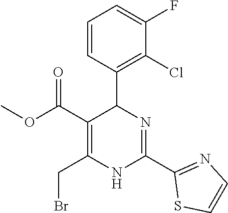 [SMILES: COC(=O)C1=C(CBr)NC(c2nccs2)=NC1c1cccc(F)c1Cl] H11-1A (single enantiomer) |
| 2-chloro-4- fluorobenzaldehyde | Ethyl 3- oxobutanoate | thiazole-2- carboximidamide | 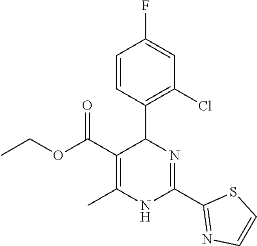 [SMILES: CCOC(=O)C1=C(C)NC(c2nccs2)=NC1c1ccc(F)cc1Cl] H12 | 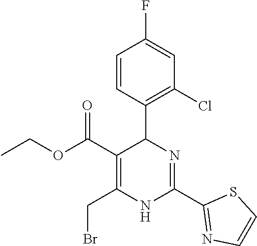 [SMILES: CCOC(=O)C1=C(CBr)NC(c2nccs2)=NC1c1ccc(F)cc1Cl] H12-1A (single enantiomer) |
| 3-fluoro-2- methylbenzaldehyde | Ethyl 3- oxobutanoate | 5-Methyloxazole-4- carboximidamide hydrochloride | 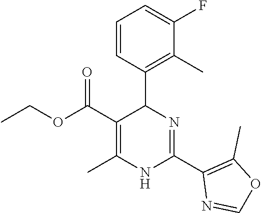 [SMILES: CCOC(=O)C1=C(C)NC(c2ncoc2C)=NC1c1cccc(F)c1C] H15 | 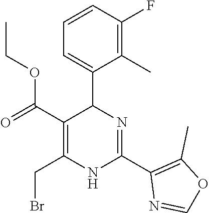 [SMILES: CCOC(=O)C1=C(CBr)NC(c2ncoc2C)=NC1c1cccc(F)c1C] H15-1A (single enantiomer) |
| 2-chloro-4- fluorobenzaldehyde | methyl 3- oxobutanoate | 5-methyloxazole-4- carboximidamide | 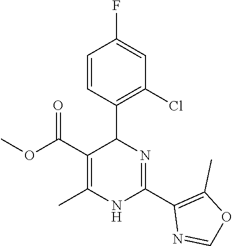 [SMILES: COC(=O)C1=C(C)NC(c2ncoc2C)=NC1c1ccc(F)cc1Cl] H18 | 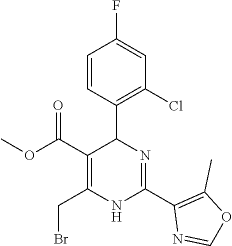 [SMILES: COC(=O)C1=C(CBr)NC(c2ncoc2C)=NC1c1ccc(F)cc1Cl] H18-1A (single enantiomer) |
| 6-fluoro-2- methylnicotinaldehyde | Ethyl 3- oxobutanoate | thiazole-2- carboximidamide | 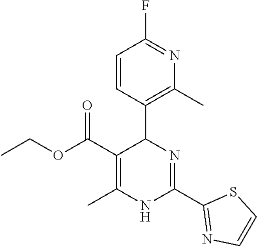 [SMILES: CCOC(=O)C1=C(C)NC(c2nccs2)=NC1c1ccc(F)nc1C] H20 | 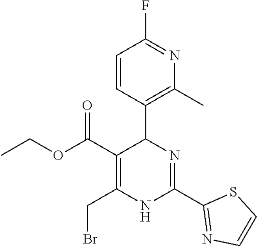 [SMILES: CCOC(=O)C1=C(CBr)NC(c2nccs2)=NC1c1ccc(F)nc1C] H20-1A (single enantiomer) |
| 3-formyl-2- methylphenyl acetate | Ethyl 3- oxobutanoate | thiazole-2- carboximidamide | 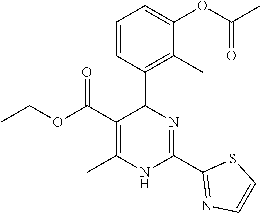 [SMILES: CCOC(=O)C1=C(C)NC(c2nccs2)=NC1c1cccc(OC(C)=O)c1C] H21 | 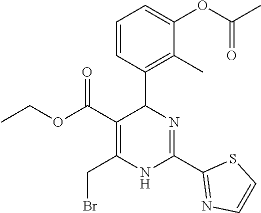 [SMILES: CCOC(=O)C1=C(CBr)NC(c2nccs2)=NC1c1cccc(OC(C)=O)c1C] H21-1A (single enantiomer) |
| 3-fluoro-2- methylbenzaldehyde | Ethyl 3- oxobutanoate | 4-methylthiazole-2- carboximidamide | 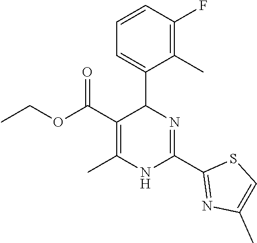 [SMILES: CCOC(=O)C1=C(C)NC(c2nc(C)cs2)=NC1c1cccc(F)c1C] H22 | 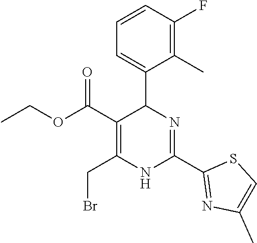 [SMILES: CCOC(=O)C1=C(CBr)NC(c2nc(C)cs2)=NC1c1cccc(F)c1C] H22-1B (single enantiomer) |
| 2,3- difluorobenzaldehyde | methyl 3- oxobutanoate | thiazole-2- carboximidamide | 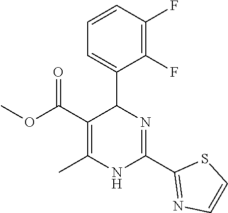 [SMILES: COC(=O)C1=C(C)NC(c2nccs2)=NC1c1cccc(F)c1F] H23 | 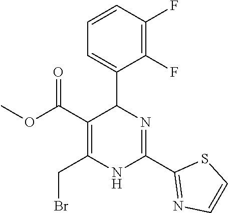 [SMILES: COC(=O)C1=C(CBr)NC(c2nccs2)=NC1c1cccc(F)c1F] H23-1A (single enantiomer) |
| 2,3- difluorobenzaldehyde | Ethyl 3- oxobutanoate | thiazole-2- carboximidamide | 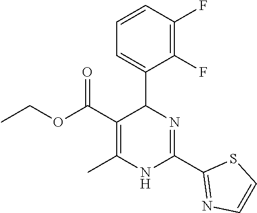 [SMILES: CCOC(=O)C1=C(C)NC(c2nccs2)=NC1c1cccc(F)c1F] H24 | 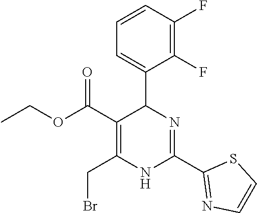 [SMILES: CCOC(=O)C1=C(CBr)NC(c2nccs2)=NC1c1cccc(F)c1F] H24-1A (single enantiomer) |
| 2,3,4- trifluorobenzaldehyde | Ethyl 3- oxobutanoate | thiazole-2- carboximidamide | 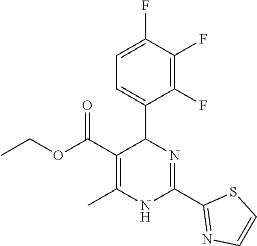 [SMILES: CCOC(=O)C1=C(C)NC(c2nccs2)=NC1c1ccc(F)c(F)c1F] H25 | 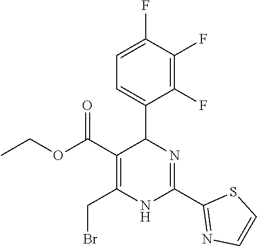 [SMILES: CCOC(=O)C1=C(CBr)NC(c2nccs2)=NC1c1ccc(F)c(F)c1F] H25-1A (single enantiomer) |
| 2,3,4- trifluorobenzaldehyde | Ethyl 3- oxobutanoate | 4-methylthiazole-2- carboximidamide | 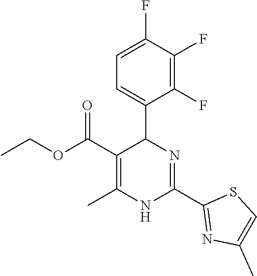 [SMILES: CCOC(=O)C1=C(C)NC(c2nc(C)cs2)=NC1c1ccc(F)c(F)c1F] H27 | 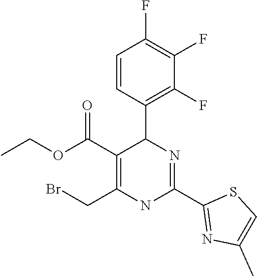 [SMILES: CCOC(=O)C1=C(CBr)NC(c2nc(C)cs2)=NC1c1ccc(F)c(F)c1F] H27-1A (single enantiomer) |
| 3-fluoro-2- methylbenzaldehyde | Ethyl 3- oxobutanoate | 1-methyl-1H- imidazole-2- carboximidamide | 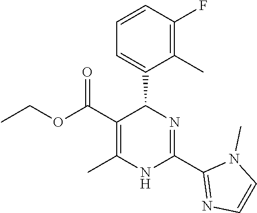 [SMILES: CCOC(=O)C1=C(C)NC(c2nccn2C)=N[C@H]1c1cccc(F)c1C] H28 | 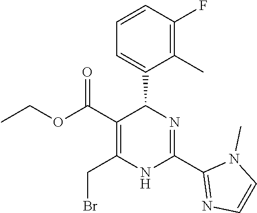 [SMILES: CCOC(=O)C1=C(CBr)NC(c2nccn2C)=N[C@H]1c1cccc(F)c1C] H28-1B (single enantiomer) |
| 4-chloro-3- fluorobenzaldehyde | Ethyl 3- oxobutanoate | 1-methyl-1H- imidazole-2- carboximidamide | 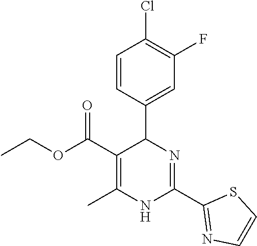 [SMILES: CCOC(=O)C1=C(C)NC(c2nccs2)=NC1c1ccc(Cl)c(F)c1] H29 | 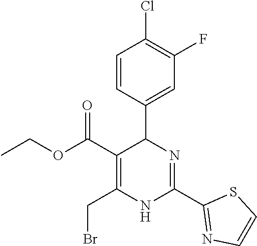 [SMILES: CCOC(=O)C1=C(CBr)NC(c2nccs2)=NC1c1ccc(Cl)c(F)c1] H29-1A (single enantiomer) |

H2: Ethyl 4-(3-fluoro-2-methylphenyl)-6-methyl-2-(thiazol-2-yl)-1,4-dihydropyrimidine-5-carboxylate was prepared using the same conditions as for H1.

¹H NMR (400 MHz, DMSO-d₆) δ 9.86 (s, 0.8H), 9.52 (d, J=2.8 Hz, 0.2H), 8.00-7.98 (in, 0.4H), 7.96 (d, J=3.2 Hz, 0.8H), 7.88 (d, J=2.8 Hz, 0.8H), 7.20-7.15 (in, 1.2H), 7.06-6.99 (in, 1.8H), 5.83 (s, 0.8H), 5.73 (d, J=3.2 Hz, 0.2H), 3.99-3.93 (m, 2H), 2.48 (s, 2.4H), 2.45 (s, 1.2H), 2.44 (s, 1.2H), 2.41 (s, 0.3H), 2.40 (s, 0.3H), 2.37 (s. 0.6H), 1.08-1.02 (in, 3H).

H2 was separated by chiral Prep-HPLC (separation condition: Column: Chiralpak OJ-H 5 μm 20*250 mm; Mobile Phase: Hex:EtOH:DEA=90:10:0.3 at 15 mL/min; Temp: 30° C.; Wavelength: 214 nm) to afford H2-A and H2-B as yellow solids.

H2-A: Chiral analysis (Column: Chiralpak OJ-H 5 μm 4.6*250 mm; Mobile Phase: Hex:EtOH:DEA=85:15:0.2 at 1.0 mL/min; Temp: 30° C.; Wavelength: 230 nm, R_{T}=7.251 min). H2-A was assigned absolute S stereochemistry by the following chemical resolution which is consistent with reported data (J. Med. Chem., 2017, 60 (8), pp 3352-3371). Optical rotation: [a]_{D}²⁰−24° (c 0.10, MeOH).

H2-B: Chiral analysis (Column: Chiralpak OJ-H 5 μm 4.6*250 mm; Mobile Phase: Hex:EtOH:DEA=85:15:0.2 at 1.0 mL/min; Temp: 30° C.; Wavelength: 230 nm, R_{T}=9.072 min). Optical rotation: [a]_{D}²⁰+35° (c 0.10, MeOH).

H2-1A: (S)-Ethyl 6-(bromomethyl)-4-(3-fluoro-2-methylphenyl)-2-(thiazol-2-yl)-1,4-dihydropyrimidine-5-carboxylate was prepared from H2-A using the same conditions as for H1-1A.

LC-MS (ESI): mass calcd. for C₁₈H₁₇BrFN₃O₂S 437.0, m/z found 440.0 [M+H]⁺. ¹H NMR (400 MHz, CDCl₃) δ 8.22 (s, 0.5H), 7.82 (d, J=3.2 Hz, 1H), 7.53 (s, 0.4H), 7.44 (s, 0.6H), 7.25-7.08 (m, 2.5H), 6.96-6.92 (s, 1H), 5.99 (s, 0.6H), 5.93 (s, 0.4H), 4.92-4.77 (m, 1.6H), 4.67-4.65 (m, 0.4H), 4.13-4.07 (m, 2H), 2.53 (s, 1.7H), 2.41 (s, 1.3H), 1.14 (t, J=7.2 Hz, 3H). Optical rotation: [a]_{D}²⁰+0.093° (c 0.10, MeOH).

H3: Methyl 4-(2-chloro-4-fluorophenyl)-6-methyl-2-(thiazol-2-yl)-1,4-dihydropyrimidine-5-carboxylate (racemic) was prepared using the same conditions as for H1.

LC-MS (ESI): mass calcd. for C₁₆H₁₃ClFN₃O₂S 365.04, m/z found 366.1 [M+H]⁺. ¹H NMR (400 MHz, CDCl₃) δ 7.84-7.83 (m, 0.9H), 7.81-7.80 (m, 0.8H), 7.55-7.50 (m, 0.6H), 7.44-7.43 (m, 0.7H), 7.33-7.26 (m, 1H), 7.13-7.11 (m, 1H), 6.95-6.88 (m, 1H), 6.18 (s, 0.7H), 6.05 (s, 0.3H), 3.63 (s, 0.8H), 3.60 (s, 2.2H), 2.57 (s, 0.8H), 2.51 (s, 2.2H).

Racemic H3 (20 g, 95% purity, 51.9 mmol) was separated by chiral Prep-HPLC (Column: Chiralpak IG 5 μm 30*250 mm; Mobile Phase: CO₂:MeOH=70:30 at 55 g/min; Col. Temp: 40° C.; Wavelength: 230 nm, Back pressure: 100 bar) to afford the title compounds H3-A (9.46 g, 95% purity from NMR, 47% yield, 100% ee) and H3-B (9.5 g, 95% purity from NMR, 48% yield, 98.0% ee) as yellow solids.

H3-A: LC-MS (ESI): mass calcd. for C₁₆H₁₃ClFN₃O₂S 365.0, m/z found 366.0. Chiral analysis (Column: Chiralpak IA 5 μm 4.6*250 mm; Mobile Phase: Hex:EtOH=80:20 at 1.0 mL/min; Temp: 30° C.; Wavelength: 254 nm, R_{T}=5.593 min). ¹H NMR (400 MHz, CDCl₃) δ 7.84-7.83 (m, 1H), 7.80 (d, J=2.8 Hz, 0.7H), 7.52-7.50 (m, 0.5H), 7.44 (d, J=2.8 Hz, 0.7H), 7.34-7.30 (m, 1H), 7.15-7.11 (m, 1H), 6.96-6.88 (m, 1H), 6.19 (s, 0.7H), 6.06 (d, J=2.4 Hz, 0.3H), 3.63 (s, 0.8H), 3.60 (s, 2.2H), 2.57 (s, 0.8H), 2.51 (s, 2.2H).

H3-B: LC-MS (ESI): mass calcd. for C₁₆H₁₃ClFN₃O₂S 365.0, m/z found 366.0. Chiral HPLC (Column: Chiralpak IA 5 μm 4.6*250 mm; Mobile Phase: Hex:EtOH=80:20 at 1.0 mL/min; Temp: 30° C.; Wavelength: 254 nm, R_{T}=6.827 min). ¹H NMR (400 MHz, CDCl₃) 7.85-7.82 (m, 1H), 7.80 (d, J=3.2 Hz, 0.7H), 7.54-7.50 (m, 0.5H), 7.43 (d, J=3.2 Hz, 0.7H), 7.34-7.30 (m, 1H), 7.14-7.11 (m, 1H), 6.96-6.88 (m, 1H), 6.18 (s, 0.7H), 6.06 (d, J=2.4 Hz, 0.3H), 3.62 (s, 0.8H), 3.60 (s, 2.2H), 2.57 (s, 0.8H), 2.50 (s, 2.2H).

H3-1A: methyl 6-(bromomethyl)-4-(2-chloro-4-fluorophenyl)-2-(thiazol-2-yl)-1,4-dihydropyrimidine-5-carboxylate was prepared from H3-A using same condition as for H1-1A.

LC-MS (ESI): mass calcd. for C₁₆H₁₂BrClFN₃O₂S 442.9, m/z found 443.9 [M+H]⁺. ¹H NMR (400 MHz, CDCl₃) δ 8.29 (br s, 0.3H), 7.84 (d, J=3.2 Hz, 1H), 7.59-7.53 (m, 1.4H), 7.47 (br s, 0.3H), 7.41-7.31 (m, 1H), 7.14 (d, J=8.4 Hz, 1H), 6.99-6.90 (m, 1H), 6.18 (s, 0.3H), 6.09 (d, J=2.0 Hz, 0.7H), 4.93 (d, J=8.4 Hz, 1H), 4.74 (d, J=11.2 Hz, 0.3H), 4.58 (d, J=8.4 Hz, 0.7H), 3.67 (s, 2.1H), 3.65 (s, 0.9H).

H4: Methyl 4-(3-fluoro-2-methylphenyl)-6-methyl-2-(thiazol-2-yl)-1,4-dihydropyrimidine-5-carboxylate (racemic) H4 was prepared using same condition as for H1. ¹H NMR (400 MHz, CDCl₃) δ 7.93 (d, J=3.2 Hz, 0.1H), 7.80-7.77 (m, 1.8H), 7.52-7.50 (m, 0.1H), 7.41 (d, J=3.2 Hz, 0.9H), 7.20 (br s, 0.1H), 7.16-7.00 (m, 2H), 6.94-6.87 (m, 1H), 6.00 (s, 0.9H), 5.90 (s, 0.1H), 3.60 (s, 3H), 2.55-2.49 (m, 5.8H), 2.40 (br s, 0.2H).

A racemic mixture of methyl 4-(3-fluoro-2-methylphenyl)-6-methyl-2-(thiazol-2-yl)-1,4-dihydropyrimidine-5-carboxylate H4 (1.30 g, 95% purity, 3.58 mmol) was separated by chiral Prep-HPLC (separation condition: Column: Chiralpak AS-H 5 μm 30*250 mm; Mobile Phase: Hex:EtOH=75:25 at 15 mL/min; Temp: 30° C.; Wavelength: 214 nm) to afford the title compounds (H4-A)(610 mg, 95% purity from ¹H NMR, 44% yield, 100% stereopure) and (H4-B) (520 mg, 95% purity from ¹H NMR, 40% yield, 97.7% stereopure) as yellow oil.

H4-A: Chiral analysis (Column: Chiralpak AS 5 μm 4.6*250 mm; Mobile Phase: Hex:EtOH=80:20 at 1 mL/min; Temp: 30° C.; Wavelength: 254 nm, R_{T}=5.247 min). ¹H NMR (400 MHz, CDCl₃) δ 7.93 (d, J=2.8 Hz, 0.1H), 7.80 (br s, 0.9H), 7.78 (d, J=2.8 Hz, 1H), 7.52-7.50 (m, 0.1H), 7.41 (d, J=3.2 Hz, 0.9H), 7.10-7.02 (m, 2H), 6.92-6.87 (m, 1H), 6.00 (s, 0.9H), 5.91 (s, 0.1H), 3.61 (s, 3H), 2.55 (s, 3H), 2.53 (s, 3H).

H4-B: Chiral analysis (Column: Chiralpak AS 5 μm 4.6*250 mm; Mobile Phase: Hex:EtOH=80:20 at 1 mL/min; Temp: 30° C.; Wavelength: 254 nm, R_{T}=9.049 min). ¹H NMR (400 MHz, CDCl₃) δ 7.78 (d, J=3.2 Hz, 2H), 7.42 (d, J=2.4 Hz, 1H), 7.10-7.05 (m, 2H), 6.92-6.89 (m, 1H), 5.99 (s, 1H), 3.61 (s, 3H), 2.54 (s, 3H), 2.53 (m, 3H).

H4-1B: Methyl 6-(bromomethyl)-4-(3-fluoro-2-methylphenyl)-2-(thiazol-2-yl)-1,4-dihydropyrimidine-5-carboxylate was prepared from H4-B using same condition as for H1-1A.

¹H NMR (400 MHz, CDCl₃) δ 8.23 (s, 1H), 7.82 (d, J=3.2 Hz, 1H), 7.53-7.44 (m, 1H), 7.12-7.07 (m, 2H), 6.93 (s, 1H), 5.98-5.94 (m, 1H), 4.89-4.66 (m, 2H), 3.65 (s, 3H), 2.53-2.41 (m, 3H).

H5: Methyl 4-(2-chloro-3,4-difluorophenyl)-6-methyl-2-(thiazol-2-yl)-1,4-dihydropyrimidine-5-carboxylate was prepared using same condition as for H1.

¹H NMR (400 MHz, CD₃OD) δ 8.08 (d, J=2.8 Hz, 0.1H), 7.98 (d, J=2.8 Hz, 0.1H), 7.93 (d, J=2.8 Hz, 0.9H), 7.72 (d, J=2.8 Hz, 0.9H), 7.26-7.18 (m, 2H), 6.13 (s, 0.9H), 6.09 (s, 0.1H), 3.61 (s, 3H), 2.53 (s, 3H).

Racemic H5 (1.10 g, 2.90 mmol) was separated by chiral Prep-HPLC (separation condition: Column: Chiralpak IC 5 μm 20*250 mm; Mobile Phase: Hex:EtOH=90:10 at 18 mL/min; Temp: 30° C.; Wavelength: 214 nm) to afford the title compounds H5-A (450 mg, 41% yield, 100% stereopure) and H5-B (450 m g, 41% yield, 99.8% stereopure) as yellow solids.

H5-A: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=6.457 min).

H5-B: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=7.641 min).

H5-1A: Methyl 6-(bromomethyl)-4-(2-chloro-3,4-difluorophenyl)-2-(thiazol-2-yl)-1,4-dihydropyrimidine-5-carboxylate was prepared from H5-A using same condition as for H1-1A.

¹H NMR (400 MHz, CD₃OD) δ 7.92 (d, J=3.2 Hz, 1H), 7.80 (d, J=3.2 Hz, 0.5H), 7.70 (d, J=3.2 Hz, 0.5H), 7.32-7.17 (m, 2H), 6.11 (s, 0.5H), 6.09 (s, 0.5H), 4.91 (d, J=10.0 Hz, 0.5H), 4.81 (d, J=10.0 Hz, 1H), 4.57 (d, J=8.4 Hz, 0.5H), 3.64 (s, 1.5H), 3.62 (s, 1.5H).

H6: Methyl 4-(3,4-difluoro-2-methylphenyl)-6-methyl-2-(thiazol-2-yl)-1,4-dihydropyrimidine-5-carboxylate was prepared using same condition as for H1. LC-MS (ESI): mass calcd. for C₁₇H₁₅F₂N₃O₂S 363.3, m/z found 364.0 [M+H]⁺. ¹H NMR (400 MHz, CDCl₃) δ 7.80-7.78 (m, 2H), 7.42 (d, J=3.2 Hz, 1H), 7.00-6.85 (m, 2H), 5.93 (s, 1H), 3.61 (s, 3H), 2.58 (s, 1.5H), 2.57 (s, 1.5H), 2.53 (s, 1.5H), 2.51 (s, 1.5H).

Racemic H6 (1.00 g, 90% purity, 2.48 mmol) was separated by chiral Prep-HPLC (separation condition: Column: Chiralpak IH 5 μm 30*250 mm; Mobile Phase: Hex:EtOH=90:10 at 18 mL/min; Temp: 30° C.; Wavelength: 214 nm) to afford the desired products H6-A (400 mg, 90% purity from ¹H NMR, 40% yield, 100% stereopure) and H6-B (400 mg, 95% purity from ¹H NMR, 42% yield, 99.9% stereopure) as yellow solids.

H6-A: Chiral analysis (Column: Chiralpak IH 5 μm 4.6*150 mm; Mobile Phase: Hex:EtOH=90:10 at 1 mL/min; Temp: 30° C.; Wavelength: 230 nm, R_{T}=4.809 min). ¹H NMR (400 MHz, CDCl₃) δ 7.84 (br s, 1H), 7.78 (d, J=3.2 Hz, 1H), 7.42 (d, J=3.2 Hz, 1H), 6.96-6.86 (m, 2H), 5.93 (s, 1H), 3.61 (s, 3H), 2.57 (d, J=1.6 Hz, 3H), 2.52 (s, 3H).

H6-B: Chiral analysis (Column: Chiralpak IH 5 μm 4.6*150 mm; Mobile Phase: Hex:EtOH=90:10 at 1 mL/min; Temp: 30° C.; Wavelength: 230 nm, R_{T}=7.018 min). ¹H NMR (400 MHz, CDCl₃) δ 7.82 (br s, 1H), 7.79 (d, J=3.2 Hz, 1H), 7.42 (d, J=3.2 Hz, 1H), 6.97-6.88 (m, 2H), 5.93 (s, 1H), 3.61 (s, 3H), 2.58 (d, J=2.0 Hz, 3H), 2.52 (s, 3H).

H6-1B: Methyl 6-(bromomethyl)-4-(3,4-difluoro-2-methylphenyl)-2-(thiazol-2-yl)-1,4-dihydropyrimidine-5-carboxylate was prepared from H6-B using same condition as for H1-1A.

¹H NMR (400 MHz, CDCl₃) δ 8.24 (s, 1H), 7.83 (d, J=3.6 Hz, 1H), 7.54-7.45 (m, 1H), 7.00-6.93 (m, 2H), 5.91 (s, 1H), 4.94-4.80 (s, 21H), 3.66 (s, 3H), 2.56-2.45 (m, 3H).

H8: Ethyl 4-(2-chloro-3,4-difluorophenyl)-6-methyl-2-(thiazol-2-yl)-1,4-dihydropyrimidine-5-carboxylate was prepared using same condition as for H1. ¹H NMR (400 MHz, CDCl₃) δ7.83-7.81 (m, 1.8H), 7.52-7.44 (m, 1.2H), 7.13-7.10 (m, 1H), 7.08-7.00 (m, 1H), 6.20 (s, 0.8H), 6.08 (s, 0.2H), 4.11-4.00 (m, 2H), 2.57 (s, 0.5H), 2.51 (s, 2.5H), 1.13 (t, J=7.2 Hz, 3H).

Racemic H8 (1.00 g, 2.51 mmol) was separated by chiral Prep-HPLC (Column: Chiralpak IC 5 μm 20*250 mm; Mobile Phase: Hex:EtOH=90:10 at 18 mL/min; Temp: 30° C.; Wavelength: 214 nm) to give the desired compound H8-A (353 mg, 35% yield, 98.1% stereopure) and H8-B (321 mg, 32% yield, 99.8% stereopure) as yellow solids.

H8-A: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=5.901 min).

H8-B: Chiral analysis (Column: Chiralpak IC 5 μm 4.6*250 mm; Mobile Phase: Hex:EtOH=90:10 at 1.0 mL/min; Temp: 30° C.; Wavelength: 254 nm, R_{T}=6.914 min).

H8-1: Ethyl 6-(bromomethyl)-4-(2-chloro-3,4-difluorophenyl)-2-(thiazol-2-yl)-1,4-dihydropyrimidine-5-carboxylate was prepared from H8 using same condition as for H1-1A.

¹H NMR (400 MHz, CDCl₃) δ 8.25 (s, 0.3H), 7.85 (d, J=2.8 Hz, 1H), 7.54-7.44 (m, 1.5H), 7.20-7.04 (m, 2.2H), 6.19-6.11 (m, 1H), 4.98-4.95 (m, 1H), 4.74-4.72 (m, 0.4H), 4.58-4.56 (m, 0.6H), 4.13-4.11 (m, 2H), 1.19-1.15 (m, 3H).

H8-1A: Ethyl 6-(bromomethyl)-4-(2-chloro-3,4-difluorophenyl)-2-(thiazol-2-yl)-1,4-dihydropyrimidine-5-carboxylate was prepared from H8-A using same condition as for H1-1A. 20 ¹H NMR (400 MHz, CDCl₃) δ 8.25 (s, 0.3H), 7.85 (d, J=3.2 Hz, 1H), 7.54 (d, J=3.2 Hz, 0.6H), 7.47-7.45 (m, 0.9H), 7.22-7.00 (m, 2.2H), 6.19 (s, 0.4H), 6.11 (d, J=2.4 Hz, 0.6H), 4.97 (d, J=11.2 Hz, 0.4H), 4.94 (d, J=8.8 Hz, 0.6H), 4.73 (d, J=11.2 Hz, 0.4H), 4.56 (d, J=8.4 Hz, 0.6H), 4.16-4.04 (m, 2H), 1.19-1.13 (m, 3H).

H9: Ethyl 4-(3,4-difluoro-2-methylphenyl)-6-methyl-2-(thiazol-2-yl)-1,4-dihydropyrimidine-5-carboxylate was prepared using same condition as for H1.

LC-MS (ESI): mass calcd. for C₁₈H₁₇F₂N₃O₂S 377.4, m/z found 378.1 [M+H]⁺. ¹H NMR (400 MHz, CDCl₃) δ 7.81-7.76 (m, 2H), 7.42 (d, J=3.2 Hz, 1H), 6.98-6.86 (m, 2H), 5.94 (s, 1H), 4.11-4.00 (m, 2H), 2.58 (s, 1.5H), 2.57 (s, 1.5H), 2.52 (s, 3H), 1.14 (t, J=7.2 Hz, 3H).

Racemic H9 (1.20 g, 90% purity, 2.86 mmol) was separated by chiral Prep-HPLC (separation condition: Column: Chiralpak IC 5 μm 30*250 mm; Mobile Phase: Hex:IPA=95:5 at 18 mL/min; Temp: 30° C.; Wavelength: 214 nm) to afford the desired compounds H9-A (580 mg, 90% purity, 48% yield, 97.8% ee) as yellow solids and H9-B (500 mg, 90% purity, 42% yield, 99.4% ee) as yellow solids.

H9-A: Chiral analysis (Column: Chiralpak IC 5 μm 4.6*250 mm; Mobile Phase: Hex:IPA=95:5 at 1 mL/min; Temp: 30° C.; Wavelength: 230 nm, R_{T}=7.550 min). ¹H NMR (400 MHz, CDCl₃) δ 7.79-7.77 (m, 2H), 7.42 (d, J=3.6 Hz, 1H), 7.00-6.88 (m, 2H), 5.94 (s, 1H), 4.08-4.01 (m, 2H), 2.58 (s, 2.5H), 2.55 (s, 0.5H), 2.52 (s, 3H), 1.14 (t, J=7.2 Hz, 3H).

H9-B: Chiral analysis (Column: Chiralpak IC 5 μm 4.6*250 mm; Mobile Phase: Hex:IPA=95:5 at 1 mL/min; Temp: 30° C.; Wavelength: 230 nm, R_{T}=8.495 min). ¹H NMR (400 MHz, CDCl₃) δ 7.79-7.75 (m, 2H), 7.42 (d, J=2.8 Hz, 1H), 6.98-6.86 (m, 2H), 5.94 (s, 1H), 4.08-4.00 (m, 2H), 2.58 (d, J=2.0 Hz, 3H), 2.52 (s, 3H), 1.14 (t, J=7.2 Hz, 3H).

H9-1A: Ethyl 6-(bromomethyl)-4-(3,4-difluoro-2-methylphenyl)-2-(thiazol-2-yl)-1,4-dihydropyrimidine-5-carboxylate was prepared from H9-A using same condition as for H1-1A.

LC-MS (ESI): mass calcd. for C₁₈H₁₆BrF₂N₃O₂S 455.0, m/z found 456.0 [M+H]⁺. ¹H NMR (400 MHz, CDCl₃) δ 7.83 (d, J=2.8 Hz, 1H), 7.54 (d, J=2.8 Hz, 0.4H), 7.44 (d, J=2.8 Hz, 0.6H), 7.21-7.06 (m, 1H), 7.02-6.89 (m, 2H), 5.93 (s, 0.6H), 5.87 (d, J=2.0 Hz, 0.4H), 4.93 (d, J=11.6 Hz, 0.6H), 4.81-4.78 (m, 1H), 4.61 (d, J=8.4 Hz, 0.4H), 4.11-4.06 (m, 2H), 2.56 (d, J=2.0 Hz, 2H), 2.45 (d, J=2.0 Hz, 1H), 1.19-1.13 (m, 3H).

H11: methyl 4-(2-chloro-3-fluorophenyl)-6-methyl-2-(thiazol-2-yl)-1,4-dihydropyrimidine-5-carboxylate was prepared using same condition as for H1.

¹H NMR (400 MHz, CDCl₃) δ 7.86 (s, 0.8H), 7.83 (d, J=2.8 Hz, 0.3H), 7.80 (d, J=2.8 Hz, 0.7H), 7.55 (s, 0.2H), 7.50 (d, J=2.8 Hz, 0.2H), 7.44 (d, J=2.8 Hz, 0.8H), 7.23-7.13 (m, 2H), 7.11-7.00 (m, 1H), 6.25 (s, 0.8H), 6.11 (d, J=1.6 Hz, 0.2H), 3.62 (s, 0.6H), 3.60 (s, 2.4H), 2.58 (s, 0.6H), 2.51 (s, 2.4H).

Racemic H11 (3.00 g, 95% purity, 7.79 mmol) was separated by chiral Prep. HPLC (Column: Chiralpak IC 5 μm 20*250 mm, Mobile Phase: Hex:IPA:DEA=90:10:0.3 at 18 mL/min, Temp: 30° C., Wavelength: 230 nm) to afford the title compounds H11-A (820 mg, 96% purity, 28% yield, 100% stereopure) and H11-B (800 mg, 97% purity, 27% yield, 99.2% stereopure) as yellow solids.

H11-A: LC-MS (ESI): mass calcd. for C₁₆H₁₃ClFN₃O₂S 365.0, m/z found 366.0 [M+H]⁺. Chiral analysis (Column: Chiralpak IC 5 μm 4.6*250 mm; Mobile Phase: Hex:IPA:DEA=90:10:0.2 at 1 mL/min; Col. Temp: 30° C.; Wavelength: 254 nm, R_{T}=10.808 min). ¹H NMR (400 MHz, CDCl₃) δ 7.86 (s, 0.7H), 7.83 (d, J=3.2 Hz, 0.2H), 7.80 (d, J=2.8 Hz, 0.8H), 7.55 (s, 0.3H), 7.50 (d, J=3.2 Hz, 0.2H), 7.44 (d, J=3.2 Hz, 0.8H), 7.22-7.13 (m, 2H), 7.08-6.99 (m, 1H), 6.25 (s, 0.8H), 6.12 (d, J=2.4 Hz, 0.2H), 3.62 (s, 1H), 3.60 (s, 2H), 2.58 (s, 1H), 2.51 (s, 2H).

Compound H11-B: LC-MS (ESI): mass calcd. for C₁₆H₁₃ClFN₃O₂S 365.0 m/z found 366.0 [M+H]⁺. Chiral analysis (Column: Chiralpak IC 5 μm 4.6*250 mm; Mobile Phase: Hex:IPA:DEA=90:10:0.2 at 1 Ll/min; Col. Temp: 30° C.; Wavelength: 254 nm, R_{T}=12.482 min). ¹H NMR (400 MHz, CDCl₃) δ 7.86 (s, 0.7H), 7.83 (d, J=3.2 Hz, 0.3H), 7.80 (d, J=3.2 Hz, 0.7H), 7.56 (s, 0.3H), 7.50 (d, J=2.8 Hz, 0.3H), 7.43 (d, J=3.2 Hz, 0.7H), 7.23-7.13 (m, 2H), 7.09-7.00 (m, 1H), 6.25 (s, 0.8H), 6.11 (d, J=2.0 Hz, 0.2H), 3.60 (s, 3H), 2.57 (s, 0.6H), 2.52 (s, 2.4H).

H11-1A: methyl 6-(bromomethyl)-4-(2-chloro-3-fluorophenyl)-2-(thiazol-2-yl)-1,4-dihydropyrimidine-5-carboxylate was prepared from H11-A using same condition as for H1-1A.

LC-MS (ESI): mass calcd. for C₁₆H₁₂BrClFN₃O₂S 442.9 m/z found 444.0 [M+H]⁺. ¹H NMR (400 MHz, DMSO-d₆) δ 8.15-7.91 (m, 2H), 7.41-7.31 (m, 2H), 7.26-7.24 (m, 1H), 6.03 (s, 1H), 4.99-4.68 (m, 2H), 3.56 (s, 3H).

H12: ethyl 4-(2-chloro-4-fluorophenyl)-6-methyl-2-(thiazol-2-yl)-1,4-dihydropyrimidine-5-carboxylate was prepared using same condition as for H1.

LC-MS (ESI): mass calcd. for C₁₇H₁₅ClFN₃O₂S 379.1, m/z found 380.0 [M+H]⁺. ¹H NMR (400 MHz, CDCl₃) δ 7.83 (d, J=3.2 Hz, 0.3H), 7.81-7.80 (m, 1.4H), 7.50 (d, J=3.6 Hz, 0.3H), 7.46 (br s, 0.3H), 7.43 (d, J=3.2 Hz, 0.7H), 7.36-7.32 (m, 1H), 7.14-7.11 (m, 1H), 6.94-6.89 (m, 1H), 6.20 (s, 0.7H), 6.08 (s, 0.3H), 4.10-4.01 (m, 2H), 2.57 (s, 0.7H), 2.51 (s, 2.3H), 1.15-1.11 (t, J=7.2 Hz, 3H).

Racemic H12 (1.00 g, 90% purity, 2.37 mmol) was separated by chiral Prep. HPLC (separation condition: Column: Chiralpak IE 5 μm 20*250 mm; Mobile Phase: Hex:EtOH=90:10 at 10 mL/min; Temp: 30° C.; Wavelength: 254 nm) to give the title compounds H12-A (400 mg, 98.1% purity, 44% yield, 100% ee) and H12-B (405 mg, 98.6% purity, 40% yield, 99.7% ee) as yellow solids.

H12-A: LC-MS (ESI): mass calcd. for C₁₇H₁₅ClFN₃O₂S 379.1, m/z found 380.1 [M+H]⁺. Chiral analysis (Column: Chiralpak IE 5 μm 4.6*250 mm; Mobile Phase: Hex:EtOH=90:10 at 1.0 mL/min; Temp: 30° C.; Wavelength: 254 nm; R_{T}=7.663 min). ¹H NMR (400 MHz, CDCl₃) δ 7.83 (d, J=3.2 Hz, 0.3H), 7.80 (d, J=2.8 Hz, 1H), 7.50 (d, J=3.2 Hz, 0.3H), 7.43 (d, J=3.2 Hz, 1H), 7.36-7.32 (m, 1H), 7.14-7.11 (m, 1H), 6.94-6.89 (m, 1H), 6.20 (s, 0.7H), 6.08 (s, 0.3H), 4.08-4.01 (m, 2H), 2.57 (s, 0.8H), 2.51 (s, 2.2H), 1.13 (t, J=7.2 Hz, 3H).

H12-B: LC-MS (ESI): mass calcd. for C₁₇H₁₅ClFN₃O₂S 379.1, m/z found 380.1 [M+H]⁺. Chiral analysis (Column: Chiralpak IE 5 μm 4.6*250 mm; Mobile Phase: Hex:EtOH=90:10 at 1.0 mL/min; Temp: 30° C.; Wavelength: 254 nm; R_{T}=9.471 min). ¹H NMR (400 MHz, CDCl₃) δ 7.83 (d, J=3.2 Hz, 0.3H), 7.80 (d, J=2.8 Hz, 1H), 7.50 (d, J=3.2 Hz, 0.3H), 7.43 (d, J=3.2 Hz, 1H), 7.36-7.32 (m, 1H), 7.14-7.11 (m, 1H), 6.94-6.89 (m, 1H), 6.20 (s, 0.7H), 6.08 (s, 0.3H), 4.08-4.00 (m, 2H), 2.57 (s, 0.8H), 2.51 (s, 2.2H), 1.13 (t, J=7.2 Hz, 3H).

H12-1A: ethyl 6-(bromomethyl)-4-(2-chloro-4-fluorophenyl)-2-(thiazol-2-yl)-1,4-dihydropyrimidine-5-carboxylate was prepared from H12-A using same condition as for H1-1A.

LC-MS (ESI): mass calcd. for C₁₇H₁₄BrClFN₃O₂S 457.0, m/z found 458.0 [M+H]⁺.

H15: Ethyl 4-(3-fluoro-2-methylphenyl)-6-methyl-2-(5-methyloxazol-4-yl)-1,4-dihydropyrimidine-5-carboxylate was prepared using same condition as for H1.

¹H NMR (400 MHz, DMSO-d₆) δ 9.21 (s, 0.8H), 8.94 (s, 0.2H), 8.35 (s, 1H), 7.16-7.06 (m, 1H), 6.99-6.94 (m, 2H), 5.80 (s, 0.8H), 5.67 (s, 0.2H), 3.97-3.94 (m, 2H), 2.46-2.40 (m, 7H), 2.38-2.30 (m, 2H), 1.04 (t, J=7.2 Hz, 3H).

Racemic H15 (1.0 g, 90% purity, 2.460 mmol) was separated by chiral Prep. HPLC (Column: Chiralpak IF 5 μm 20*250 mm, Mobile Phase: Hex:EtOH=98:2 at 18 mL/min, Temp: 30° C., Wavelength: 254 nm) to afford the title compounds H15-A (461 mg, 95% purity from ¹H NMR, 46% yield, 100% stereopure) as yellow solids and H15-B (466 mg, 95% purity from NMR, 47% yield, 99.0% stereopure) as yellow solids.

H15-A: LC-MS (ESI): mass calcd. for C₁₉H₂₀FN₃O₃ 357.1, m/z found 358.1 [M+H]⁺. Chiral analysis (Column: Chiralpak IF 5 μm 4.6*250 mm; Mobile Phase: Hex:EtOH=98:2 at 1 mL/min; Col. Temp: 30° C.; Wavelength: 254 nm, R_{T}=10.686 min). ¹H NMR (400 MHz, CDCl₃) δ 7.66 (s, 1H), 7.51 (s, 1H), 7.09-7.04 (m, 1H), 7.00-6.93 (m, 1H), 6.88 (t, J=8.8 Hz, 1H), 5.98 (s, 1H), 4.07-3.98 (m, 2H), 2.54 (s, 5H), 2.51 (s, 4H), 1.11 (t, J=7.2 Hz, 3H).

H15-B: LC-MS (ESI): mass calcd. for C₁₉H₂₀FN₃O₃ 357.1 m/z found 358.1 [M+H]⁺. Chiral analysis (Column: Chiralpak IF 5 μm 4.6*250 mm; Mobile Phase: Hex:EtOH=98:2 at 1 ml/min; Col. Temp: 30° C.; Wavelength: 254 nm, R_{T}=13.222 min). ¹H NMR (400 MHz, CDCl₃) δ 7.66 (s, 1H), 7.51 (s, 1H), 7.09-7.04 (m, 1H), 7.00-6.98 (m, 1H), 6.88 (t, J=8.4 Hz, 1H), 5.98 (s, 1H), 4.08-4.01 (m, 2H), 2.55 (s, 5H), 2.51 (s, 4H), 1.11 (t, J=6.8 Hz, 3H).

H15-1A: Ethyl 4-(3-fluoro-2-methylphenyl)-6-methyl-2-(5-methyloxazol-4-yl)-1,4-dihydropyrimidine-5-carboxylate was prepared from H15-A using same condition as for H1-1A.

LC-MS (ESI): mass calcd. for C₁₉H₁₉BrFN₃O₃ 435.0 m/z found 438.1 [M+H]⁺. ¹H NMR (400 MHz, CDCl₃) δ 7.67 (s, 1H), 7.17-7.06 (m, 1H), 7.00-6.85 (m, 2H), 5.89 (br s, 1H), 4.75 (br s, 2H), 4.08 (q, J=6.8 Hz, 2H), 2.85-2.70 (m, 2H), 2.64-2.04 (m, 4H), 1.13 (t, J=7.2 Hz, 3H).

H18: Methyl 4-(2-chloro-4-fluorophenyl)-6-methyl-2-(5-methyloxazol-4-yl)-1,4-dihydropyrimidine-5-carboxylate was made using the same conditions as for H1.

LC-MS (ESI): mass calcd. for C₁₇H₁₅ClFN₃O₃ 363.1, m/z found 364.0 [M+H]⁺. ¹H NMR (400 MHz, CDCl₃) δ 7.67 (s, 1H), 7.59-7.50 (m, 0.6H), 7.34-7.31 (m, 0.8H), 7.23-7.14 (m, 0.6H), 7.13 (dd, J=8.4, 2.0 Hz, 1H), 6.96-6.86 (m, 1H), 6.14 (s, 0.6H), 5.99 (s, 0.4H), 3.60 (s, 3H), 2.72 (s, 1.2H), 2.62-2.51 (m, 4.8H).

Chiral Separation of H18:

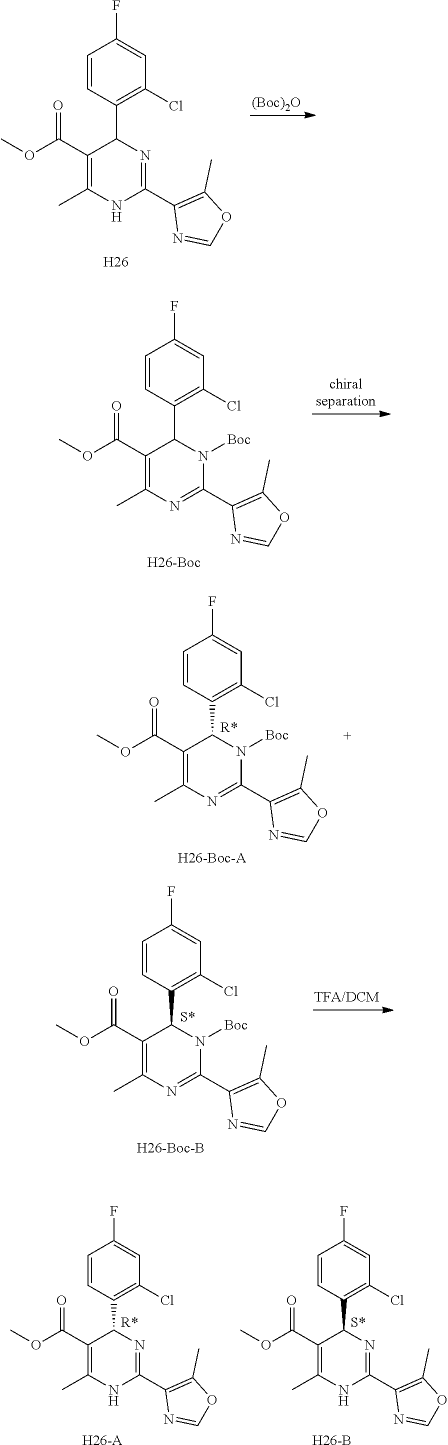

Preparation of H18-Boc: To a solution of H18 (13.1 g, 90% purity, 32.4 mmol) in tetrahydrofuran (200 mL) was added di-tert-butyl dicarbonate (14.2 g, 64.9 mmol) and N,N-dimethylpyridin-4-amine (3.97 g, 32.5 mmol). After stirred at 55° C. for 2 hours, the mixture was concentrated to give a residue, which was purified by silica gel column chromatography (petroleum ether:ethyl acetate=10:1 to 6:1) to afford the title compound (12.1 g, 90% purity from ¹H NMR, 72% yield) as yellow solids. LC-MS (ESI): mass calcd. for C₂₂H₂₃ClFN₃O₅ 463.1, m/z found 464.1 [M+H]⁺. ¹H NMR (400 MHz, CDCl₃) δ 7.68 (s, 1H), 7.17-7.10 (m, 2H), 6.79 (td, J=8.4, 2.4 Hz, 1H), 6.70 (s, 1H), 3.71 (s, 3H), 2.58 (s, 3H), 2.44 (s, 3H), 1.34 (s, 9H).

Racemic H18-Boc (15.2 g, 90% purity, 29.5 mmol) was separated by prep. chiral HPLC (Chiral Column: Chiralpak IC 5 μm 30*250 mm; Mobile Phase: Hex:EtOH=98:2 at 30 mL/min; Temp: 30° C.; Wavelength: 254 nm) to give H18-Boc-A (6.58 g, 95% purity from ¹H NMR, 99.5% ee, 46% yield) as yellow solids and H18-Boc-B (5.76 g, 95% purity from 1H NMR, 97.9% ee, 40% yield) as yellow solids.

H18-Boc-A: LC-MS (ESI): mass calcd. for C₂₂H₂₃ClFN₃O₅ 463.1, m/z found 464.0 [M+H]⁺. Chiral analysis (Chiral Column: Chiralpak IC 5 μm 4.6*250 mm; Mobile Phase: Hex:EtOH=98:2 at 1 mL/min; Temp: 30° C.; Wavelength: 254 nm; R_{T}=10.327 min). ¹H NMR (400 MHz, CDCl₃) δ 7.68 (s, 1H), 7.17-7.10 (m, 2H), 6.79 (td, J=8.0, 2.4 Hz, 1H), 6.70 (s, 1H), 3.71 (s, 3H), 2.58 (s, 3H), 2.44 (s, 3H), 1.34 (s, 9H).

H18-Boc-B: LC-MS (ESI): mass calcd. for C₂₂H₂₃ClFN₃O₅ 463.1, m/z found 464.0 [M+H]⁺. Chiral analysis (Column: Chiralpak IC 5 μm 4.6*250 mm; Mobile Phase: Hex:EtOH=98:2, at 1 mL/min; Temp: 30° C.; Wavelength: 254 nm; R_{T}=11.793 min). ¹H NMR (400 MHz, CDCl₃) δ 7.68 (s, 1H), 7.17-7.10 (m, 2H), 6.79 (td, J=8.0, 2.4 Hz, 1H), 6.70 (s, 1H), 3.71 (s, 3H), 2.57 (s, 3H), 2.43 (s, 3H), 1.34 (s, 9H).

Deprotection of H18-Boc-A to give H18-A: LC-MS (ESI): mass calcd. for C₁₇H₁₅ClFN₃O₃ 363.1, m/z found 364.1 [M+H]⁺. ¹H NMR (400 MHz, CDCl₃) δ 7.67 (s, 1H), 7.61-7.52 (s, 0.7H), 7.38-7.28 (m, 0.6H), 7.26-7.22 (m, 0.7H), 7.13 (dd, J=8.8, 2.8 Hz, 1H), 6.91-6.85 (m, 1H), 6.14 (s, 0.7H), 5.99 (s, 0.3H), 3.60 (s, 3H), 2.72 (s, 0.9H), 2.64-2.51 (m, 5.1H).

H18-1A: Methyl 6-(bromomethyl)-4-(2-chloro-4-fluorophenyl)-2-(5-methyloxazol-4-yl)-1,4-dihydropyrimidine-5-carboxylate was made from H18-A using same condition as for H1-1A.

LC-MS (ESI): mass calcd. for C₁₇H₁₄BrClFN₃O₃ 441.0, m/z found 442.0 [M+H]⁺. ¹H NMR (400 MHz, CDCl₃) δ 7.68 (s, 1H), 7.40-7.34 (m, 1H), 7.14 (dd, J=8.4, 2.4 Hz, 1H), 6.98-6.94 (m, 1H), 6.02 (s, 1H), 4.89 (d, J=8.4 Hz, 1H), 4.64 (d, J=8.4 Hz, 1H), 3.65 (s, 3H), 2.76 (s, 3H).

H20: ethyl 4-(6-fluoro-2-methylpyridin-3-yl)-6-methyl-2-(thiazol-2-yl)-1,4-dihydropyrimidine-5-carboxylate was prepared using same condition as for H1.

LC-MS (ESI): mass calcd. for C₁₇H₁₇FN₄O₂S 360.1, m/z found 361.3 [M+H]⁺. ¹H NMR (400 MHz, CDCl₃) δ 7.83 (s, 1H), 7.81 (d, J=3.2 Hz, 1H), 7.60 (t, J=8.0 Hz, 1H), 7.45 (d, J=3.2 Hz, 1H), 6.68 (dd, J=8.4, 3.2 Hz, 1H), 5.98 (s, 1H), 4.11-4.03 (m, 2H), 2.80 (s, 3H), 2.53 (s, 3H), 1.15 (t, J=7.2 Hz, 3H).

Racemic H20 was chiral separated to give H20-A and H20-B.

H20-A: LC-MS (ESI): mass calcd. for C₁₇H₁₇FN₄O₂S 360.11, m/z found 361.3 [M+H]⁺. ¹H NMR (400 MHz, CDCl₃) δ 7.83 (s, 1H), 7.80 (d, J=3.2 Hz, 1H), 7.60 (t, J=8.0 Hz, 1H), 7.45 (d, J=3.2 Hz, 1H), 6.68 (dd, J=8.4, 3.2 Hz, 1H), 5.98 (s, 1H), 4.11-4.03 (m, 2H), 2.80 (s, 3H), 2.53 (s, 3H), 1.15 (t, J=7.2 Hz, 3H). Chiral analysis (100% stereopure, Chiralpak IE m 4.6*250 mm; Mobile Phase: Hex:EtOH=70:30 at 1 mL/min; Temp: 30° C., Wavelength: 254 nm, R_{T}=5.773 min).

H20-B: LC-MS (ESI): mass calcd. for C₁₇H₁₇FN₄O₂S 360.11, m/z found 361.3 [M+H]⁺. ¹H NMR (400 MHz, CDCl₃) δ 7.83 (s, 1H), 7.80 (d, J=3.2 Hz, 1H), 7.60 (t, J=8.0 Hz, 1H), 7.45 (d, J=3.2 Hz, 1H), 6.68 (dd, J=8.0, 3.2 Hz, 1H), 5.98 (s, 1H), 4.11-4.03 (m, 2H), 2.80 (s, 3H), 2.53 (s, 3H), 1.15 (t, J=6.8 Hz, 3H). Chiral analysis (99.9% stereopure, Chiralpak IE 5 μm 4.6*250 mm; Mobile Phase: Hex:EtOH=70:30 at 1 mL/min; Temp: 30° C., Wavelength: 254 nm, R_{T}=6.724 min).

H20-1A: ethyl 6-(bromomethyl)-4-(6-fluoro-2-methylpyridin-3-yl)-2-(thiazol-2-yl)-1,4-dihydropyrimidine-5-carboxylate was prepared from H20-A using same condition as for H1-1A. LC-MS (ESI): mass calcd. for C₁₇H₁₆BrFN₄O₂S 438.0, m/z found 441.2 [M+H]⁺. ¹H NMR (400 MHz, CDCl₃) δ 8.32-8.18 (m, 0.4H), 7.84 (d, J=3.2 Hz, 1H), 7.73-7.59 (m, 0.7H), 7.54-7.44 (m, 1H), 6.76-6.69 (m, 1H), 5.02-4.85 (m, 1H), 4.79-4.61 (m, 0.3H), 4.16-4.05 (m, 2H), 2.83-2.65 (s, 3H), 1.17 (t, J=7.2 Hz, 3H).

H21: ethyl 4-(3-acetoxy-2-methylphenyl)-6-methyl-2-(thiazol-2-yl)-1,4-dihydropyrimidine-5-carboxylate (racemic) was prepared using the same conditions as for H1.

LC-MS (ESI): mass calcd. for C₂₀H₂₁N₃O₄S 399.1, m/z found 400.1 [M+H]⁺. ¹H NMR (400 MHz, CDCl₃) δ 7.77 (d, J=8.8 Hz, 1H), 7.40 (d, J=3.2 Hz, 1H), 7.18-7.11 (m, 2H), 6.91-6.89 (m, 1H), 6.03 (s, 1H), 4.08-4.02 (m, 2H), 2.53 (s, 3H), 2.46 (s, 3H), 2.34 (s, 3H), 1.13-1.09 (m, 3H).

Racemic H21 (740 mg, 90% purity, 1.67 mmol) was separated by chiral Prep. SFC (Column: Chiralpak IG 5 μm 20*250 mm; Mobile Phase: CO₂:MeOH=70:30 at 50 g/min; Col. Temp: 40° C.; Wavelength: 214 nm, Back pressure: 100 bar) to give title compound H21-A (240 mg, 90% purity from ¹H NMR, 32% yield, 100% ee) and H21-B (270 mg, 90% purity from ¹H NMR, 36% yield, 97.5% ee) as yellow solids.

H21-A: LC-MS (ESI): mass calcd. for C₂₀H₂₁N₃O₄S 399.1, m/z found 400.3 [M+H]⁺. Chiral analysis (Method: Chiralpak IG 5 μm 4.6*250 mm; Mobile Phase: CO₂:MeOH=70:30 at 3 g/min; Col. Temp: 40° C.; Wavelength: 214 nm, Back pressure: 100 bar, R_{T}=2.80 min). ¹H NMR (400 MHz, CDCl₃) δ 7.78-7.76 (m, 1H), 7.41-7.40 (m, 1H), 7.18-7.11 (m, 2H), 6.91-6.89 (m, 1H), 6.03 (s, 0.9H), 5.94 (s, 0.1H), 4.09-3.99 (m, 2H), 2.53 (s, 3H), 2.46 (s, 3H), 2.34 (s, 3H), 1.13-1.09 (m, 3H).

H21-B: LC-MS (ESI): mass calcd. for C₂₀H₂₁N₃O₄S 399.1, m/z found 400.3 [M+H]⁺. Chiral analysis (Method: Chiralpak IG 5 μm 4.6*250 mm; Mobile Phase: CO₂:MeOH=70:30 at 3 g/min; Col. Temp: 40° C.; Wavelength: 214 nm, Back pressure: 100 bar, R_{T}=3.57 min). ¹H NMR (400 MHz, CDCl₃) δ 7.78-7.76 (m, 1H), 7.41-7.40 (m, 1H), 7.18-7.11 (m, 2H), 6.94-6.89 (m, 1H), 6.03 (s, 0.9H), 5.94 (s, 0.1H), 4.09-3.99 (m, 2H), 2.53 (s, 3H), 2.46 (s, 3H), 2.34 (s, 3H), 1.13-1.08 (m, 3H).

H21-1A: ethyl 4-(3-acetoxy-2-methylphenyl)-6-(bromomethyl)-2-(thiazol-2-yl)-1,4-dihydropyrimidine-5-carboxylate was prepared from H21-A using same condition as for H1-1A.

¹H NMR (400 MHz, CDCl₃) δ 8.18 (s, 0.5H), 7.82 (d, J=3.2 Hz, 1H), 7.52 (d, J=3.2 Hz, 0.5H), 7.42 (d, J=3.2 Hz, 0.5H), 7.37 (d, J=7.6 Hz, 0.5H), 7.23-7.13 (m, 2H), 6.96-6.91 (m, 1H), 6.01 (s, 0.5H), 5.96 (s, 0.5H), 4.92-4.85 (m, 1H), 4.77-4.68 (m, 1H), 4.09-4.04 (m, 2H), 2.44 (s, 1.5H), 2.34 (s, 3H), 2.31 (s, 1.5H), 1.15-1.11 (m, 3H).

H22: ethyl 4-(3-fluoro-2-methylphenyl)-6-methyl-2-(4-methylthiazol-2-yl)-1,4-dihydropyrimidine-5-carboxylate was prepared using the same conditions as for H1.

¹H NMR (400 MHz, CDCl₃) δ 7.76 (br s, 1H), 7.11-7.02 (m, 2H), 6.96 (s, 1H), 6.91-6.87 (m, 1H), 5.99 (s, 1H), 4.09-4.01 (m, 2H), 2.53 (s, 6H), 2.43 (s, 3H), 1.12 (t, J=7.2 Hz, 3H).

Racemic H22 (1.0 g, 90% purity, 2.41 mmol) was separated by chiral Prep. HPLC (Column: Chiralpak IH 5 μm 20*250 mm; Mobile Phase: Hex:EtOH=90:10 at 15 mL/min; Temp: 30° C.; Wavelength: 254 nm) to afford the title compounds H22-A (440 mg, 90% purity from ¹H NMR, 44% yield, 99.9% stereopure) and H22-B (420 mg, 90% purity from ¹H NMR, 42% yield, 99.8% stereopure) as yellow solids.

H22-A: Chiral analysis (Column: Chiralpak IH 5 μm 4.6*250 mm; Mobile Phase: Hex:EtOH=90:10 at 1.0 mL/min; Temp: 30° C.; Wavelength: 254 nm, R_{T}=5.205 min). ¹H NMR (400 MHz, CDCl₃) δ 7.77 (s, 1H), 7.05-6.88 (m, 4H), 5.98 (s, 1H), 4.09-3.99 (m, 2H), 2.53 (s, 6H), 2.43 (s, 3H), 1.12 (t, J=7.2 Hz, 3H).

H22-B: Chiral analysis (Column: Chiralpak IH 5 μm 4.6*250 mm; Mobile Phase: Hex:EtOH=90:10 at 1.0 mL/min; Temp: 30° C.; Wavelength: 254 nm, R_{T}=6.494 min). ¹H NMR (400 MHz, CDCl₃) δ 7.77 (s, 1H), 7.06-6.87 (m, 4H), 5.98 (s, 1H), 4.09-4.01 (m, 2H), 2.53 (s, 6H), 2.43 (s, 3H), 1.12 (t, J=7.2 Hz, 3H).

H22-1B: ethyl 6-(bromomethyl)-4-(3-fluoro-2-methylphenyl)-2-(4-methylthiazol-2-yl)-1,4-dihydropyrimidine-5-carboxylate was made from H22-B using same condition as for H1-1A.

LC-MS (ESI): mass calcd. for C₁₉H₁₉BrFN₃O₂S 451.0, m/z found 454.4 [M+H]⁺.

H23: methyl 4-(2,3-difluorophenyl)-6-methyl-2-(thiazol-2-yl)-1,4-dihydropyrimidine-5-carboxylate was made using the same conditions as for H1.

¹H NMR (400 MHz, CD₃OD) δ 7.94 (d, J=3.2 Hz, 1H), 7.74 (d, J=3.2 Hz, 1H), 7.19-7.08 (m, 3H), 6.03 (s, 1H), 3.63 (s, 3H), 2.50 (s, 3H).

Racemic H23 (6.20 g, 90% purity, 15.9 mmol) was separated by chiral Prep. HPLC (Column: Chiralpak IE 5 μm 30*250 mm; Mobile Phase: Hex:EtOH:DEA=80:20:0.3 at 25 mL/min; Temp: 30° C.; Wavelength: 230 nm) to afford the title compound H23-A (2.70 g, 90% purity from ¹H NMR, 44% yield, 100 stereopure) and H23-B (2.80 g, 90% purity from ¹H NMR, 45% yield, 99.8% stereopure) as yellow solids.

H23-A: Chiral analysis (Column: Chiralpak IE 5 μm 4.6*250 mm; Mobile Phase: Hex:EtOH:DEA=80:20:0.2 at 1.0 mL/min; Temp: 30° C.; Wavelength: 254 nm, R_{T}=6.542 min). ¹H NMR (400 MHz, CD₃OD) δ 7.82 (d, J=2.8 Hz, 1H), 7.62 (d, J=2.4 Hz, 1H), 7.05-6.96 (m, 3H), 5.91 (s, 1H), 3.51 (s, 3H), 2.38 (s, 3H).

H23-B: Chiral analysis (Column: Chiralpak IE 5 μm 4.6*250 mm; Mobile Phase: Hex:EtOH:DEA=80:20:0.2 at 1.0 mL/min; Temp: 30° C.; Wavelength: 254 nm, R_{T}=7.723 min). ¹H NMR (400 MHz, CD₃OD) δ 7.94 (d, J=3.2 Hz, 1H), 7.73 (d, J=3.2 Hz, 1H), 7.18-7.08 (m, 3H), 6.03 (s, 1H), 3.63 (s, 3H), 2.50 (s, 3H).

H23-1A was prepared from H23-A using same condition as for H1-1A.

LC-MS (ESI): mass calcd. for C₁₆H₁₂BrF₂N₃O₂S 427.0, m/z found 430.2 [M+H]⁺. ¹H NMR (400 MHz, CD₃OD) δ 7.96 (s, 1H), 7.84 (d, J=2.0 Hz, 0.5H), 7.76 (d, J=2.4 Hz, 0.5H), 7.24-7.12 (m, 3H), 6.04 (d, J=6.0 Hz, 1H), 4.89 (s, 1H), 4.80 (d, J=8.4 Hz, 0.5H), 4.65 (d, J=8.4 Hz, 0.5H), 3.69 (s, 3H).

H24: Ethyl 4-(2,3-difluorophenyl)-6-methyl-2-(thiazol-2-yl)-1,4-dihydropyrimidine-5-carboxylate was made using the same conditions as for H1.

LC-MS (ESI): mass calcd. for C₁₇H₁₅F₂N₃O₂S 363.1, m/z found 364.1 [M+H]⁺. ¹H NMR (400 MHz, CDCl₃) δ 7.90-7.82 (m, 2H), 7.52 (d, J=3.2 Hz, 0.1H), 7.45 (d, J=3.2 Hz, 0.8H), 7.33 (s, 0.1H), 7.13-7.07 (m, 1H), 7.00-6.96 (m, 2H), 6.11 (s, 0.9H), 6.05 (d, J=2.4 Hz, 0.1H), 4.07 (q, J=7.2 Hz, 2H), 2.54 (s, 0.4H), 2.47 (s, 2.6H), 1.17 (t, J=7.2 Hz, 3H).

Racemic H24 (1.7 g, 90% purity, 4.67 mmol) was separated by chiral prep HPLC (Column: Chiralpak IC 5 μm 30*250 mm; Mobile Phase: Hex:EtOH=90:10 at 30 mL/min; Temp: 30° C.; Wavelength: 254 nm) to give the title compounds H24-A (730 mg, 90% purity from ¹H NMR, 43% yield, 99.8% stereopure) and H24-B (740 mg, 90% purity from ¹H NMR, 43% yield, 98.8% stereopure) as yellow solids.

H24-A: LC-MS (ESI): mass calcd. for C₁₇H₁₅F₂N₃O₂S 363.1, m/z found 364.1 [M+H]⁺. Chiral analysis (Column: Chiralpak IC 5 μm 4.6*250 mm; Mobile Phase: Hex:EtOH=90:10 at 1 mL/min; Temp: 30° C.; Wavelength: 254 nm, R_{T}=7.389 min). ¹H NMR (400 MHz, CDCl₃) δ 7.90-7.82 (m, 2H), 7.52 (s, 0.1H), 7.45 (d, J=3.6 Hz, 0.9H), 7.13-6.96 (m, 2H), 6.11 (s, 0.9H), 6.05 (s, 0.1H), 4.07 (q, J=7.2 Hz, 2H), 2.54 (s, 0.4H), 2.47 (s, 2.6H), 1.17 (t, J=7.2 Hz, 3H).

H24-B: LC-MS (ESI): mass calcd. for C₁₇H₁₅F₂N₃O₂S 363.1, m/z found 364.1 [M+H]⁺. Chiral analysis (Column: Chiralpak IC 5 μm 4.6*250 mm; Mobile Phase: Hex:EtOH=90:10 at 1 mL/min; Temp: 30° C.; Wavelength: 254 nm, R_{T}=8.894 min). ¹H NMR (400 MHz, CDCl₃) δ 7.90-7.82 (m, 2H), 7.52 (s, 0.1H), 7.45 (d, J=3.6 Hz, 0.9H), 7.13-6.98 (m, 2H), 6.11 (s, 0.9H), 6.05 (s, 0.1H), 4.07 (q, J=7.2 Hz, 2H), 2.54 (s, 0.4H), 2.47 (s, 2.6H), 1.17 (t, J=7.2 Hz, 3H).

H24-1A: Ethyl 6-(bromomethyl)-4-(2,3-difluorophenyl)-2-(thiazol-2-yl)-1,4-dihydropyrimidine-5-carboxylate was made using the same conditions as for H1-1A.

¹H NMR (400 MHz, CDCl₃) δ 8.30 (s, 0.5H), 7.86-7.84 (m, 1H), 7.55-7.48 (m, 1H), 7.38 (s, 0.5H), 7.19-6.99 (m, 3H), 6.10-6.07 (m, 1H), 4.85-4.79 (m, 1.5H), 4.64-4.61 (m, 0.5H), 4.17-4.09 (m, 2H), 1.19 (t, J=7.2 Hz, 3H).

H25: Ethyl 6-methyl-2-(thiazol-2-yl)-4-(2,3,4-trifluorophenyl)-1,4-dihydropyrimidine-5-carboxylate was made using the same conditions as for H1.

LC-MS (ESI): mass calcd. for C₁₇H₁₄F₃N₃O₂S 381.4, m/z found 382.2 [M+H]⁺.

Racemic H25 (3.20 g, 90% purity, 0.755 mmol) was separated by chiral Prep. HPLC (Column: Chiralpak IE 5 μm 4.6*250 mm; Mobile Phase: Hex:EtOH=90:10 at 20 mL/min; Temp: 30° C.; Wavelength: 254 nm) to give the title compounds H25-A (990 mg, 90% purity by ¹H NMR, 31% yield, 100% stereopure) and H25-B (980 mg, 90% purity by ¹H NMR, 31% yield, 98.9% stereopure) as yellow oil.

H25-A: Chiral analysis (Column: Chiralpak IE 5 μm 4.6*250 mm; Mobile Phase: Hex:EtOH=90:10 at 1 mL/min, Temp: 30° C.; Wavelength: 254 nm, R_{T}=7.555 min). ¹H NMR (300 MHz, CDCl₃) δ 7.92 (br s, 1H), 7.87 (d, J=3.3 Hz, 1H), 7.51 (d, J=3.0 Hz, 1H), 7.15-7.07 (m, 1H), 6.97-6.88 (m, 1H), 6.09 (s, 1H), 4.20-4.15 (m, 2H), 2.52 (s, 3H), 1.30 (t, J=7.2 Hz, 3H).

H25-B: Chiral analysis (Column: Chiralpak IE 5 μm 4.6*250 mm; Mobile Phase: Hex:EtOH=90:10 at 1 mL/min, Temp: 30° C.; Wavelength: 254 nm, R_{T}=8.572 min). ¹H NMR (400 MHz, CDCl₃) δ 7.87 (br s, 1H), 7.83 (d, J=6.8 Hz, 1H), 7.47 (d, J=6.8 Hz, 1H), 7.07-7.05 (m, 1H), 6.90-6.87 (m, 1H), 6.05 (s, 1H), 4.11-4.06 (m, 2H), 2.48 (s, 3H), 1.18 (t, J=7.2 Hz, 3H).

H25-1A: Ethyl 6-(bromomethyl)-2-(thiazol-2-yl)-4-(2,3,4-trifluorophenyl)-1,4-dihydropyrimidine-5-carboxylate was made using the same conditions as for H1-1A.

¹H NMR (400 MHz, CDCl₃) δ 8.25 (br s, 1H), 7.79 (d, J=2.4 Hz, 1H), 7.53-7.40 (m, 1H), 7.10-6.95 (m, 1H), 6.93-6.76 (m, 1H), 5.96 (s, 1H), 4.79-4.45 (m, 2H), 4.08-4.04 (m, 2H), 1.13 (t, J=7.2 Hz, 3H).

H27: ethyl 6-methyl-2-(4-methylthiazol-2-yl)-4-(2,3,4-trifluorophenyl)-1,4-dihydropyrimidine-5-carboxylate was made using the same conditions as for H1.

¹H NMR (400 MHz, CDCl₃) δ 7.87 (s, 1H), 7.08-7.03 (m, 1H), 7.00 (s, 1H), 6.91-6.84 (m, 1H), 6.03 (s, 1H), 4.08 (q, J=7.2 Hz, 2H), 2.47 (s, 3H), 2.45 (s, 3H), 1.18 (t, J=7.2 Hz, 3H).

Racemic H27 (4.50 g, 90% purity, 10.2 mmol) was separated by chiral prep. HPLC (Column IC 5 μm 30*250 mm, Mobile phase: Hex:EtOH=95:5 at 30 mL/min; Temp: 25° C.; Wavelength: 254 nm) to give the title compounds H27-A (1.80 g, 95.4% purity, 96% stereopure, 40% yield) and H27-B (1.71 g, 99.2% purity, 99.9% stereopure, 38% yield) as yellow solids.

H27-A: LC-MS (ESI): mass calcd. for C₁₈H₁₆F₃N₃O₂S 395.4, m/z found 396.1 [M+H]⁺. Chiral analysis (Column: Chiralpak IC 5 μm 4.6*250 mm; Mobile Phase: Hex:EtOH=95:5 at 1 mL/min; Temp: 30° C.; Wavelength: 254 nm, R_{T}=7.170 min). ¹H NMR (400 MHz, CDCl₃) δ 7.88 (s, 1H), 7.08-7.05 (m, 1H), 7.00 (s, 1H), 6.88-6.86 (m, 1H), 6.03 (s, 1H), 4.05 (q, J=7.2 Hz, 2H), 2.47 (s, 3H), 2.45 (s, 3H), 1.18 (t, J=7.2 Hz, 3H).

H27-B: LC-MS (ESI): mass calcd. for C₁₈H₁₆F₃N₃O₂S 395.4, m/z found 396.1 [M+H]⁺. 35 Chiral analysis (Column: Chiralpak IC 5 μm 4.6*250 mm; Mobile Phase: Hex:EtOH=95:5 at 1 mL/min; Temp: 30° C.; Wavelength: 254 nm, R_{T}=9.445 min). ¹H NMR (400 MHz, CDCl₃) δ 7.87 (s, 1H), 7.08-7.02 (m, 1H), 7.00 (s, 1H), 6.90-6.84 (m, 1H), 6.03 (s, 1H), 4.12-4.05 (m, 2H), 2.47-2.44 (m, 6H), 1.17 (t, J=7.2 Hz, 3H).

H27-1A: ethyl 6-(bromomethyl)-2-(4-methylthiazol-2-yl)-4-(2,3,4-trifluorophenyl)-1,4-dihydropyrimidine-5-carboxylate was made using the same conditions as for H1-1A.

LC-MS (ESI): mass calcd. for C₁₈H₁₅BrF₃N₃O₂S 473.0, m/z found 474.5 [M+H]⁺. ¹H NMR (400 MHz, CDCl₃) δ 7.16-6.86 (m, 4H), 6.03-6.00 (m, 1H), 4.85 (d, J=10.0 Hz, 1H), 4.77 (d, J=11.2 Hz, 0.5H), 4.57 (d, J=8.4 Hz, 0.5H), 4.15-4.10 (m, 2H), 2.48 (s, 1.5H), 2.44 (s, 1.5H), 1.20 (t, J=7.2 Hz, 3H).

H28: Ethyl 4-(3-fluoro-2-methylphenyl)-6-methyl-2-(1-methyl-1H-imidazol-2-yl)-1,4-dihydropyrimidine-5-carboxylate was made using the same conditions as for H1.

¹H NMR (400 MHz, CDCl₃) δ 7.98 (s, 1H), 7.10-7.04 (m, 1H), 7.01-6.96 (m, 2H), 6.91-6.87 (m, 2H), 6.01 (s, 1H), 4.09-4.03 (m, 2H), 3.91 (s, 3H), 2.54 (s, 3H), 2.52 (s, 3H), 1.12 (t, J=7.2 Hz, 3H).

Racemic H28 (750 mg, 90% purity, 1.89 mmol) was separated by chiral Prep. HPLC (Column: Chiralpak AS-H 5 μm 30*250 mm; Mobile Phase: Hex:EtOH=95:5 at 15 mL/min; Temp: 30° C.; Wavelength: 214 nm) to give the title compounds H28-A (250 mg, 90% purity from ¹H NMR, 33% yield, 99.8% stereopure) and H28-B (240 mg, 90% purity from ¹H NMR, 32% yield, 98.5% stereopure) as yellow solids.

H28-A: LC-MS (ESI): mass calcd. for C₁₉H₂₁FN₄O₂ 356.2, m/z found 357.5 [M+H]⁺. Chiral analysis (Column: Chiralpak IC 5 μm 4.6*250 mm; Mobile Phase: Hex:EtOH=95:5 at 1 mL/min; Temp: 30° C.; Wavelength: 214 nm, R_{T}=6.886 min). ¹H NMR (400 MHz, CDCl₃) δ 7.99 (s, 1H), 7.12-7.08 (m, 1H), 7.06-7.00 (m, 1H), 6.96 (s, 1H), 6.91-6.87 (m, 2H), 6.01 (s, 1H), 4.11-4.01 (m, 2H), 3.91 (s, 3H), 2.54 (s, 3H), 2.52 (s, 3H), 1.12 (t, J=7.2 Hz, 3H).

H28-B: LC-MS (ESI): mass calcd. for C₁₉H₂₁FN₄O₂ 356.2, m/z found 357.5 [M+H]⁺. Chiral analysis (Column: Chiralpak IC 5 μm 4.6*250 mm; Mobile Phase: Hex:EtOH=95:5 at 1 mL/min; Temp: 30° C.; Wavelength: 214 nm, R_{T}=8.988 min). ¹H NMR (400 MHz, CDCl₃) δ 7.99 (s, 1H), 7.10-7.06 (m, 1H), 7.04-7.02 (m, 1H), 7.00 (s, 1H), 6.91-6.85 (m, 2H), 6.01 (s, 1H), 4.09-4.01 (m, 2H), 3.91 (s, 3H), 2.53 (s, 3H), 2.52 (s, 3H), 1.12 (t, J=7.2 Hz, 3H).

H28-1B: Ethyl 6-(bromomethyl)-4-(3-fluoro-2-methylphenyl)-2-(1-methyl-1H-imidazol-2-yl)-1,4-dihydropyrimidine-5-carboxylate was made using the same conditions as for H1-1A.

LC-MS (ESI): mass calcd. for C₁₉H₂₀BrFN₄O₂ 434.1, m/z found 435.3 [M+H]⁺. ¹H NMR (400 MHz, CDCl₃) δ 7.17-7.00 (m, 3H), 6.94-6.89 (m, 2H), 6.04-5.87 (m, 1H), 4.26-3.85 (m, 7H), 2.53-2.41 (m, 3H), 1.26-1.12 (m, 3H).

H29: ethyl 4-(4-chloro-3-fluorophenyl)-6-methyl-2-(thiazol-2-yl)-1,4-dihydropyrimidine-5-carboxylate was made using the same conditions as for H1.

¹H NMR (400 MHz, CDCl₃) δ 7.88 (br s, 1H), 7.84 (d, J=3.2 Hz, 1H), 7.48 (d, J=3.2 Hz, 1H), 7.30 (t, J=7.8 Hz, 1H), 7.19-7.13 (m, 2H), 5.80 (s, 1H), 4.14 (q, J=7.2 Hz, 2H), 2.47 (s, 3H), 1.23 (t, J=7.0 Hz, 3H).

Racemic H29 (1.66 g, 90% purity, 3.93 mmol) was separated by chiral prep. HPLC (Column: Chiralpak IC 5 μm 30*250 mm; Mobile Phase: Hex:EtOH:DEA=95:5:0.2 at 25 mL/min; Temp: 30° C.; Wavelength: 254 nm) to give the title products H29-A (710 mg, 90% purity from NMR, 43% yield, 99.9% stereopure) and H29-B (730 mg, 90% purity from NMR, 44% yield, 99.4% stereopure) as yellow solids.

H29-A: Chiral analysis (Column: Chiralpak IC 5 μm 4.6*250 mm; Mobile Phase: Hex:EtOH:DEA=95:5:0.2 at 1.0 mL/min; Temp: 30° C.; Wavelength: 254 nm, R_{T}=8.074 min). ¹H NMR (300 MHz, CDCl₃) δ 7.94 (br s, 1H), 7.89 (d, J=3.3 Hz, 1H), 7.53 (d, J=3.0 Hz, 1H), 7.35 (t, J=7.7 Hz, 1H), 7.23-7.16 (m, 2H), 5.84 (s, 1H), 4.18 (q, J=7.2 Hz, 2H), 2.51 (s, 3H), 1.27 (t, J=7.1 Hz, 3H).

H29-B: Chiral analysis (Column: Chiralpak IC 5 μm 4.6*250 mm; Mobile Phase: Hex:EtOH:DEA=95:5:0.2 at 1.0 mL/min; Temp: 30° C.; Wavelength: 254 nm, R_{T}=10.030 min). ¹H NMR (300 MHz, CDCl₃) δ 7.95 (br s, 1H), 7.89 (d, J=3.3 Hz, 1H), 7.53 (d, J=3.3 Hz, 1H), 7.35 (t, J=7.8 Hz, 1H), 7.24-7.17 (m, 2H), 5.84 (s, 1H), 4.19 (q, J=7.1 Hz, 2H), 2.52 (s, 3H), 1.28 (t, J=7.2 Hz, 3H).

H29-1A: ethyl 6-(bromomethyl)-4-(4-chloro-3-fluorophenyl)-2-(thiazol-2-yl)-1,4-dihydropyrimidine-5-carboxylate was made using the same conditions as for H1-1A.

LC-MS (ESI): mass calcd. for C₁₇H₁₄BrClFN₃O₂S 457.0, m/z found 458.0 [M+H]⁺.

### Compound 1A: 3-(4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylpropanoic acid

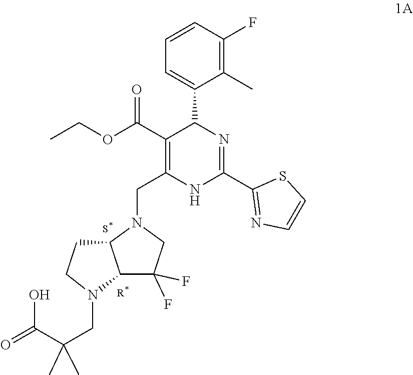

### Preparation of Intermediate S1

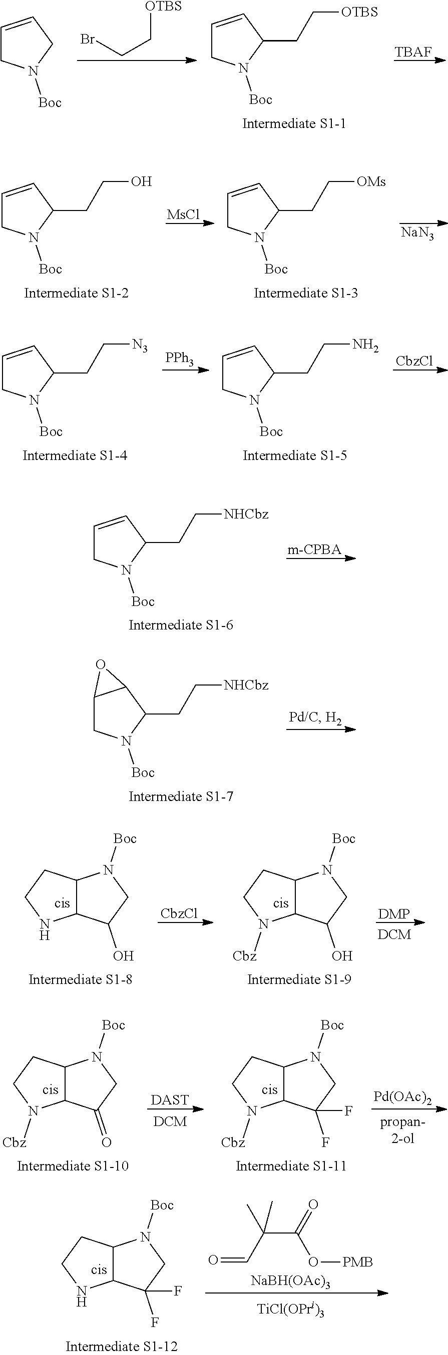

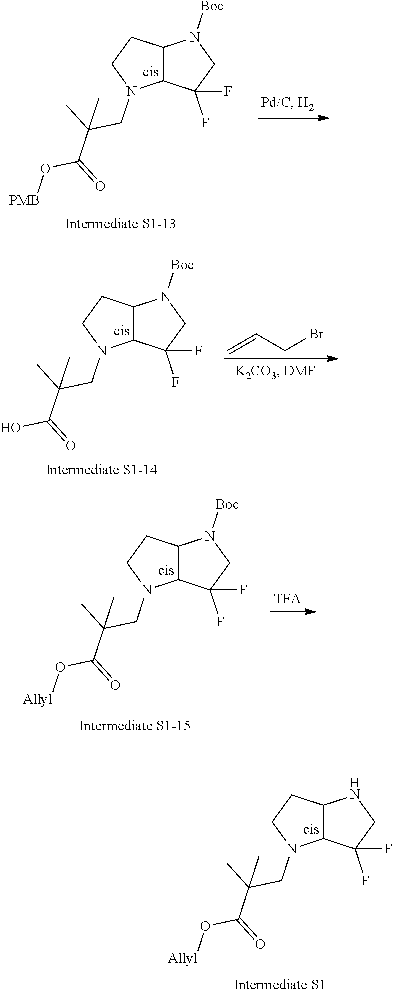

### S1-1: tert-butyl 2-(2-((tert-butyldimethylsilyl)oxy)ethyl)-2,5-dihydro-1H-pyrrole-1-carboxylate

To a solution of tert-butyl 2,5-dihydro-1H-pyrrole-1-carboxylate (50 g, 296 mmol) in THE (500 mL) at −78° C. was added LDA (384 mL, 1 M in THF, 384 mmol). After addition the mixture was stirred at −78° C. for 1 hour. Then (2-bromoethoxy)(tert-butyl)dimethylsilane (77 g, 325 mmol) was added at −78° C. and the mixture was stirred at −78° C. for 30 min. The mixture was poured into water (500 mL) and extracted with EtOAc (500 mL×2). The combined organic layers were washed with brine (300 mL), dried over Na₂SO₄ and concentrated. The residue was purified by column chromatography (PE/EtOAc=50/1) to give product (51 g, 53%) as yellow oil. LC-MS (ESI): mass calcd. for C₁₇H₃₃NO₃Si 327.2 m/z; Found 228 [M+H−100]⁺.

### S1-2: tert-butyl 2-(2-hydroxyethyl)-2,5-dihydro-1H-pyrrole-1-carboxylate

A mixture of tert-butyl 2-(2-((tert-butyldimethylsilyl)oxy)ethyl)-2,5-dihydro-1H-pyrrole-1-carboxylate S1-1 (77 g, 235 mmol) in TBAF/THF (1 M, 258 mL) was stirred at room temperature for 1 hour. The mixture was poured into water (300 mL) and extracted with EtOAc (1000 mL). The organic layer was washed with brine (300 mL×4), dried over Na₂SO₄ and concentrated. The residue was purified by column chromatography (PE/EtOAc=10/1) to give product (47 g, 94%) as yellow oil. ¹H NMR (400 MHz, CDCl₃) δ 5.78-5.73 (m, 2H), 4.74-4.73 (m, 1H), 4.26-4.22 (m, 1H), 4.01-3.97 (m, 1H), 3.66-3.64 (m, 2H), 1.95-1.87 (m, 1H), 1.51-1.47 (m, 10H).

### S1-3: tert-butyl 2-(2-((methylsulfonyl)oxy)ethyl)-2,5-dihydro-1H-pyrrole-1-carboxylate

To a mixture of tert-butyl 2-(2-hydroxyethyl)-2,5-dihydro-1H-pyrrole-1-carboxylate S1-2 (37 g, 174 mmol) and TEA (52.6 g, 521 mmol) in DCM (400 mL) was added MsCl (29.8 g, 261 mmol). Then the mixture was stirred at 0° C. for 1 hour. The mixture was poured into water (300 mL) and extracted with DCM (500 mL). The organic layer was washed with brine (300 mL), dried over Na₂SO₄, concentrated to give product (50 g, 100%) as colorless oil. LC-MS (ESI): mass calcd. for C₁₂H₂₁NO₅S 291.1 m/z found 192 [M+H−100]⁺.

### S1-4: tert-butyl 2-(2-azidoethyl)-2,5-dihydro-1H-pyrrole-1-carboxylate

A mixture of tert-butyl 2-(2-((methylsulfonyl)oxy)ethyl)-2,5-dihydro-1H-pyrrole-1-carboxylate S1-3 (24.6 g, 67.6 mmol) and NaN₃ (25 g, 338 mmol) in DMF (200 mL) was stirred at 50° C. for 5 hours under N₂. The mixture was poured into EtOAc (800 mL). The organic layer was washed water (200 mL*5). The organic layer was concentrated to give crude product (20 g, 100%) as yellow oil. LC-MS (ESI): mass calcd. for C₁₁H₁₈N₄O₂ 238.1 m/z found 139 [M+H−100]⁺.

### S1-5: tert-butyl 2-(2-aminoethyl)-2,5-dihydro-1H-pyrrole-1-carboxylate

A mixture of crude tert-butyl 2-(2-azidoethyl)-2,5-dihydro-1H-pyrrole-1-carboxylate S1-4 (20 g, 84 mmol) and PPh₃ (22 g, 84 mmol) in THF/H₂O (200 mL/20 mL) was stirred at 45° C. for 3 hours under N₂. The mixture was concentrated to give crude product (40 g, 100%) as yellow oil. LC-MS (ESI): mass calcd. for C₁₁H₂₀N₂O₂ 212.2 m/z found 213.2 [M+H]⁺.

### S1-6: tert-butyl 2-(2-(((benzyloxy)carbonyl)amino)ethyl)-2,5-dihydro-1H-pyrrole-1-carboxylate

To a mixture of tert-butyl 2-(2-aminoethyl)-2,5-dihydro-1H-pyrrole-1-carboxylate S1-5 (40 g crude, 84 mmol) and Na₂CO₃ (23 g, 168 mmol) in dioxane/H₂O (180 mL/40 mL) was added CbzCl (17.2 g, 100 mmol) dropwise at 0° C. Then the mixture was stirred at room temperature overnight. The mixture was poured into water (300 mL), extracted with EtOAc (800 mL). The organic layer was washed with brine (300 mL), dried over Na₂SO₄ and concentrated. The crude was purified by column chromatography (PE/EtOAc=3/1) to give product (19 g, 63%) as yellow oil. ¹H NMR (400 MHz, CDCl3) δ 7.37-7.26 (m, 6H), 5.80-5.73 (m, 2H), 5.13-5.08 (m, 2H), 4.64-4.54 (m, 1H), 4.23-4.16 (m, 1H), 4.00-3.95 (m, 1H), 3.37-3.09 (m, 2H), 1.89-1.77 (m, 1H), 1.69-1.64 (m, 1H), 1.46 (s, 9H).

### S1-7: tert-butyl 2-(2-(((benzyloxy)carbonyl)amino)ethyl)-6-oxa-3-azabicyclo[3.1.0]hexane-3-carboxylate

To a mixture of tert-butyl 2-(2-(((benzyloxy)carbonyl)amino)ethyl)-2,5-dihydro-1H-pyrrole-1-carboxylate S1-6 (19 g, 54.8 mmol) in DCM (200 mL) was added mCPBA (19 g, 109 mmol). The mixture was stirred at room temperature overnight. The mixture was poured into water (300 mL) and extracted with EtOAc (300 mL). The organic solution was washed with Na₂CO₃ solution (sat., 300 mL) and concentrated. The residue was purified by column chromatography (PE/EtOAc=2/1) to give product (19.8 g, 100%) as colorless oil. ¹H NMR (400 MHz, CDCl₃) δ 7.39-7.26 (m, 6H), 6.00-5.74 (m, 1H), 5.15-5.05 (m, 2H), 4.20-2.96 (m, 7H), 1.82-1.77 (m, 1H), 1.46 (s, 9H).

### S1-8: tert-butyl 3-hydroxyhexahydropyrrolo[3,2-b]pyrrole-1(2H)-carboxylate

A mixture of tert-butyl 2-(2-(((benzyloxy)carbonyl)amino)ethyl)-6-oxa-3-azabicyclo-[3.1.0]hexane-3-carboxylate S1-7 (31 g, 85.6 mmol) and Pd/C (10%, 6.2 g) in EtOH (600 mL) was stirred at 30° C. overnight under H₂ (1 atm). The mixture was filtered and the filtrate was concentrated to give crude product (19 g, 97%) as colorless oil. LC-MS (ESI): mass calcd. for C₁₁H₂₀N₂O₃ 228.1 m/z found 129 [M+H−100]⁺.

### S1-9: 4-benzyl 1-(tert-butyl) 3-hydroxyhexahydropyrrolo[3,2-b]pyrrole-1,4-dicarboxylate

To a mixture of tert-butyl 3-hydroxyhexahydropyrrolo[3,2-b]pyrrole-1(2H)-carboxylate S1-8 (19 g, 83.3 mmol) and Na₂CO₃ (23 g, 167 mmol) in dioxane/H₂O (190 mL/50 mL) was added CbzCl (172 g, 100 mmol) dropwise at 0° C. Then the mixture was stirred at room temperature for 3 hours. The mixture was poured into water (500 mL) and extracted with EtOAc (300 mL×2). The organic solution was concentrated and the residue was purified by column chromatography (PE/EtOAc=2/1) to give product (20.5 g, 68%) as colorless oil. ¹H NMR (400 MHz, DMSO-d₆) δ 7.37-7.30 (m, 5H), 5.30-5.26 (m, 1H), 5.13-5.03 (m, 2H), 4.33-4.32 (m, 1H), 4.16-4.13 (m, 1H), 4.04-4.00 (m, 1H), 3.66-3.61 (m, 1H), 3.46-3.40 (m, 1H), 3.16-3.01 (m, 2H), 2.03-1.84 (m, 2H), 1.40 (s, 9H).

### S1-10: 4-benzyl 1-(tert-butyl) 3-oxohexahydropyrrolo[3,2-b]pyrrole-1,4-dicarboxylate

To a solution of 4-benzyl 1-(tert-butyl) 3-hydroxyhexahydropyrrolo[3,2-b]pyrrole-1,4-dicarboxylate S1-9 (220 mg, 0.61 mmol) in dichloromethane (8 mL) was added Dess-Martin periodinane (580 mg, 0.57 mmol). The mixture was stirred at room temperature overnight. The reaction mixture was quenched with saturated sodium thiosulfate solution (20 mL) and saturated sodium bicarbonate solution (20 mL), then extracted with ethyl acetate (50 mL) twice. The combined organic layers were washed with brine (50 mL), dried over Na₂SO₄ and filtered. The filtrate was concentrated under reduced pressure to give the crude product. The crude was purified by column chromatography on silica gel (dichloromethane:methanol=30:1) to give the desired product (170 mg, 70% yield, 90% purity by HNMR) as a colorless oil. ¹H NMR (400 MHz, CDCl₃) δ 7.44-7.29 (m, 5H), 5.18 (d, J=5.2 Hz, 2H), 4.80-4.22 (m, 2H), 4.13-3.85 (m, 1H), 3.74-3.55 (m, 1H), 3.49-3.22 (m, 2H), 2.35-2.19 (m, 1H), 2.10-1.81 (m, 1H), 1.49-1.46 (m, 9H).

### S1-11: 4-benzyl 1-(tert-butyl) 3,3-difluorohexahydropyrrolo[3,2-b]pyrrole-1,4-dicarboxylate

To a solution of diethylaminosulfur trifluoride (820 mg, 5.09 mmol) in dichloromethane (40 mL) was added 4-benzyl 1-(tert-butyl) 3-oxohexahydropyrrolo[3,2-b]pyrrole-1,4-dicarboxylate S1-10 (500 mg, 1.25 mmol, 90% purity). After the addition, the mixture was stirred at room temperature for 12 hours. The reaction mixture was quenched with saturated sodium bicarbonate solution (50 mL) and extracted with ethyl acetate (50 mL) twice. The combined organic layers were washed with brine (50 mL), dried over Na₂SO₄ and filtered. The filtrate was concentrated under reduced pressure to give the crude product. The crude was purified by column chromatography on silica gel (petroleum ether:ethyl acetate=2:1) to give the desired product (260 mg, 49% yield, 90% purity by HNMR) as white solid. ¹H NMR (400 MHz, CDCl₃) δ 7.46-7.30 (m, 5H), 5.17 (s, 2H), 4.70-4.45 (m, 2H), 4.05-3.75 (m, 2H), 3.60-3.44 (m, 1H), 3.38-3.25 (m, 1H), 2.21-1.94 (m, 2H), 1.47 (s, 9H).

### S1-12: tert-butyl 3,3-difluorohexahydropyrrolo[3,2-b]pyrrole-1(2H)-carboxylate

To a mixture of 4-benzyl 1-(tert-butyl) 3,3-difluorohexahydropyrrolo[3,2-b]pyrrole-1,4-dicarboxylate S1-11 (260 mg, 0.61 mmol, 90% purity) in isopropyl alcohol (15 mL) was added palladium acetate (100 mg) and activated carbon (15 mg). The mixture was stirred at 50° C. overnight under hydrogen (50 psi). The reaction mixture was filtered and the filtrate was concentrated under reduced pressure to give the desired product (260 mg, 95% yield, 90% purity by LCMS) as a colorless oil. The crude was used in next step without purification. ¹H NMR (400 MHz, CDCl₃) δ 4.50-4.35 (m, 1H), 3.96-3.63 (m, 4H), 3.61-3.38 (m, 1H), 3.11-3.06 (m, 1H), 2.98-2.91 (m, 1H), 2.10-1.98 (m, 1H), 1.40 (s, 9H).

S1-12 (1.8 g, 4.472 mmol, 95% purity) was separated by chiral prep. HPLC (Column: Chiralpak IB 5 μm 4.6*250 mm; Mobile Phase: Hex:EtOH=80:20, 20 mL/min; Temp: 30° C.; Wavelength: 214) to afford S1-12A (780 mg, 46% yield, 100% purity from LCMS) as a yellow oil and S1-12B (780 mg, 44% yield, 97% purity from LCMS) as a yellow oil.

S1-12A: LC-MS (ESI): mass calcd. for C₁₉H₂₄F₂N₂O₄ 382.4, m/z found 383.2 [M+H]⁺; Chiral HPLC: chiralpak IB Sum, 4.6*250 mm; phase: Hex:EtOH=80:20; F: 1 mL/min; W=230 nm; T=30° C.; Rt=4.839 min, 100% ee.

S1-12B: LC-MS (ESI): mass calcd. for C₁₉H₂₄F₂N₂O₄ 382.4, m/z found 383.2 [M+H]⁺. Chiral HPLC: chiralpak IB 5 um, 4.6*250 mm; phase: Hex:EtOH=80:20; F: 1 mL/min; W=230 nm; T=30° C.; Rt=5.515 min, 99.8% ee.

### S1-13: tert-butyl 3,3-difluoro-4-(3-((4-methoxybenzyl)oxy)-2,2-dimethyl-3-oxopropyl)-hexahydropyrrolo[3,2-b]pyrrole-1(2H)-carboxylate

To a mixture of tert-butyl 3,3-difluorohexahydropyrrolo[3,2-b]pyrrole-1(2H)-carboxylate S1-12 (45 mg, 0.16 mmol, 90% purity) and 4-methoxybenzyl 2,2-dimethyl-3-oxopropanoate (90 mg, 0.34 mmol, 90% purity) in dichloromethane (10 mL) was added titanium(IV) chloride tripropan-2-olate (200 mg, 0.77 mmol) and acetic acid (0.1 mL). The mixture was stirred at 30° C. for 1 hour and then sodium triacetoxyborohydride (115 mg, 0.54 mmol) was added. After addition, the mixture was stirred at 30° C. overnight. The reaction mixture was concentrated under reduced pressure to give the crude product. The crude was purified by C18 column (acetonitrile:water=1:20 to 3:1) to give the desired product (55 mg, 64% yield, 90% purity by HNMR) as white solids. ¹H NMR (400 MHz, CDCl₃) δ 7.29-7.27 (m, 2H), 6.88 (d, J=8.0 Hz, 2H), 5.09-4.97 (m, 2H), 4.41-4.24 (m, 1H), 3.81 (s, 3H), 3.79-3.54 (m, 2H), 3.16-3.10 (m, 1H), 2.96-2.90 (m, 1H), 2.86-2.79 (m, 2H), 2.27-2.20 (m, 1H), 2.17-2.04 (m, 1H), 1.84-1.70 (m, 1H), 1.45 (s, 9H), 1.21 (s, 3H), 1.16 (s, 3H).

### S1-14: 3-(4-(tert-butoxycarbonyl)-6,6-difluorohexahydropyrrolo[3,2-b]pyrrol-1(2H)-yl)-2,2-dimethylpropanoic acid

To a mixture of tert-butyl 3,3-difluoro-4-(3-((4-methoxybenzyl)oxy)-2,2-dimethyl-3-oxopropyl)-hexahydropyrrolo[3,2-b]pyrrole-1(2H)-carboxylate S1-13 (95 mg, 0.18 mmol, 90% purity) in isopropyl alcohol (15 mL) was added 10% wt. palladium on charcoal (30 mg). The mixture was stirred at 50° C. overnight under hydrogen (50 psi). The reaction mixture was filtered and the filtrate was concentrated under reduced pressure to give the desired product (70 mg, 99% yield, 90% purity by HNMR) as a colorless oil. ¹H NMR (400 MHz, CDCl₃) δ 4.52-4.36 (m, 1H), 3.91-3.76 (m, 1H), 3.72-3.60 (m, 1H), 3.32-3.20 (m, 2H), 3.00-2.94 (m, 1H), 2.80-2.76 (m, 1H), 2.59-2.45 (m, 1H), 2.32-2.20 (m, 1H), 2.02-1.84 (m, 1H), 1.46 (s, 9H), 1.25 (s, 6H).

### S1-15: tert-butyl 4-(3-(allyloxy)-2,2-dimethyl-3-oxopropyl)-3,3-difluorohexahydro pyrrolo[3,2-b]-pyrrole-1(2H)-carboxylate

To a mixture of 3-(4-(tert-butoxycarbonyl)-6,6-difluorohexahydropyrrolo[3,2-b]pyrrol-1(2H)-yl)-2,2-dimethylpropanoic acid S1-14 (80 mg, 0.17 mmol, 90% purity) and potassium carbonate (75 mg, 0.54 mmol) in N,N-dimethylformamide (6 mL) was added 3-bromoprop-1-ene (50 mg, 0.41 mmol). The mixture was stirred at room temperature for 3 hours. The reaction mixture was concentrated under reduced pressure to give the crude product. The crude was purified by column chromatography on silica gel (petroleum ether:ethyl acetate=4:1) to give the desired product (50 mg, 68% yield, 90% purity by HNMR) as yellow oil. ¹H NMR (400 MHz, CDCl3) δ 5.97-5.87 (m, 1H), 5.35-5.22 (m, 2H), 4.56-4.54 (m, 2H), 4.45-4.27 (m, 1H), 3.89-3.59 (m, 2H), 3.22-3.16 (m, 1H), 3.08-3.02 (m, 1H), 2.90-2.82 (m, 2H), 2.43-2.23 (m, 1H), 2.13-1.95 (m, 1H), 1.72-1.57 (m, 1H), 1.46 (s, 9H), 1.25 (s, 3H), 1.23 (s, 3H).

### S1: Allyl 3-(6,6-difluorohexahydropyrrolo[3,2-b]pyrrol-1(2H)-yl)-2,2-dimethylpropanoate

To a mixture of cis-tert-butyl 4-(3-(allyloxy)-2,2-dimethyl-3-oxopropyl)-3,3-difluorohexahydro-pyrrolo[3,2-b]pyrrole-1(2H)-carboxylate S1-15 (50 mg, 0.12 mmol, 90% purity) in ethyl acetate (4 mL) was added hydrogen chloride in ethyl acetate (0.5 mL, 4 M).

The mixture was stirred at room temperature for 3 hours. The reaction mixture was concentrated under reduced pressure to give the desired product (40 mg, 98% yield, 92% purity by LCMS) as white solids. LC-MS (ESI): mass calcd. for C₁₄H₂₂F₂N₂O₂ 288.2, m/z found 289.1 [M+H]⁺.

### Compound 1A-1: (4S)-ethyl 6-((4-(3-(allyloxy)-2,2-dimethyl-3-oxopropyl)-3,3-difluorohexahydropyrrolo[3,2-b]-pyrrol-1(2H)-yl)methyl)-4-(3-fluoro-2-methylphenyl)-2-(thiazol-2-yl)-1,4-dihydropyrimidine-5-carboxylate

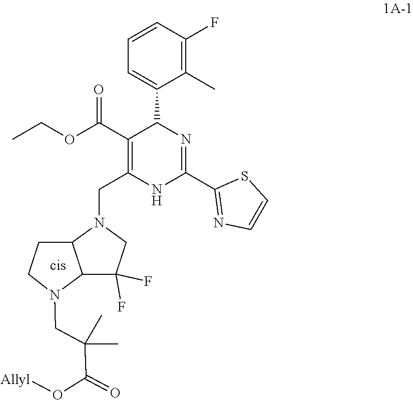

Typical Coupling Method 1:

To a mixture of allyl 3-(6,6-difluorohexahydropyrrolo[3,2-b]pyrrol-1(2H)-yl)-2,2-dimethyl-propanoate S1 (40 mg, 0.11 mmol, 92% purity) in dichloromethane (10 mL) was added 2,2′,2″-nitrilotriethanol (185 mg, 1.24 mmol) and (S)-ethyl 6-(bromomethyl)-4-(3-fluoro-2-methylphenyl)-2-(thiazol-2-yl)-1,4-dihydropyrimidine-5-carboxylate H2-1A (60 mg, 0.12 mmol, 90% purity). The mixture was stirred at 35° C. overnight. The reaction mixture was concentrated under reduced pressure to give the crude product. The crude was purified by C18 column (acetonitrile:water=1:20 to 2:1) to give the desired product (40 mg, 58% yield, 95% purity by LCMS) as yellow solid. LC-MS (ESI): R_{T}=2.25 min, mass calcd. for C₃₂H₃₈F₃N₅O₄S 645.3, m/z found 646.1 [M+H]⁺.

### Compound 1A-2: (S)-ethyl 6-((4-(3-(allyloxy)-2,2-dimethyl-3-oxopropyl)-3,3-difluorohexahydro-pyrrolo[3,2-b]pyrrol-1(2H)-yl)methyl)-4-(3-fluoro-2-methylphenyl)-2-(thiazol-2-yl)-1,4-dihydropyrimidine-5-carboxylate

(4S)-ethyl 6-((4-(3-(allyloxy)-2,2-dimethyl-3-oxopropyl)-3,3-difluorohexahydropyrrolo-[3,2-b]pyrrol-1(2H)-yl)methyl)-4-(3-fluoro-2-methylphenyl)-2-(thiazol-2-yl)-1,4-dihydropyrimidine-5-carboxylate (Compound 1A-1) was purified by chiral HPLC (Chiralpak IG 5 um 30*250 mm; Mobile Phase: methanol=100 at 25 mL/min; Temp: 30° C.; Wavelength: 254 nm) to give compound 1A-2 (17 mg, 47% yield, 90% purity by HNMR) as yellow solid. H NMR (400 MHz, CDCl₃) δ 9.38 (br s, 1H), 7.84 (d, J=2.8 Hz, 1H), 7.41 (s, 1H), 7.09-7.04 (m, 1H), 6.98 (d, J=7.6 Hz, 1H), 6.92-6.88 (m, 1H), 6.01 (s, 1H), 5.98-5.87 (m, 1H), 5.33 (d, J=17.2 Hz, 1H), 5.22 (d, J=10.4 Hz, 1H), 4.57 (d, J=5.6 Hz, 2H), 4.27-4.22 (m, 1H), 4.09-3.97 (m, 3H), 3.76-3.68 (m, 1H), 3.56-3.44 (m, 1H), 3.32-2.80 (m, 5H), 2.65-2.58 (m, 1H), 2.54 (s, 3H), 1.90-1.78 (m, 2H), 1.26 (s, 3H), 1.22 (s, 3H), 1.11 (t, J=6.8 Hz, 3H).

### Compound 1A: 3-(4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylpropanoic acid

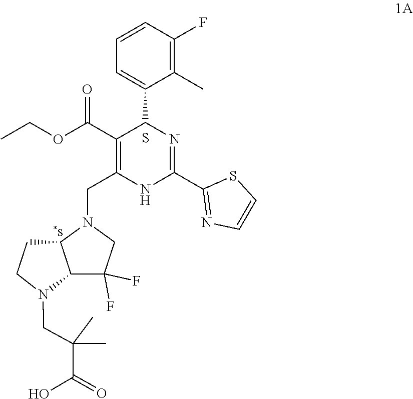

To a mixture of (S)-ethyl 6-((4-(3-(allyloxy)-2,2-dimethyl-3-oxopropyl)-3,3-difluorohexa-hydropyrrolo[3,2-b]pyrrol-1(2H)-yl)methyl)-4-(3-fluoro-2-methylphenyl)-2-(thiazol-2-yl)-1,4-dihydropyrimidine-5-carboxylate (Compound 1A-2, 17 mg, 0.021 mmol, 90% purity) and pyrrolidine (10 mg, 0.14 mmol) in dichloromethane (5 mL) was added tetrakis(triphenylphosphine)palladium (10 mg, 0.009 mmol). The mixture was stirred at 28° C. for 4 hours. The reaction mixture was concentrated under reduced pressure to give the crude product. The crude was purified by C18 column (acetonitrile:water=1:20 to 2:1) to give the desired product (7.8 mg, 60% yield, 99.9% purity by LCMS) as yellow solids. LC-MS (ESI): mass calcd. for C₂₉H₃₄F₃N₅O₄S 605.2, m/z found 606.3 [M+H]⁺. 1 H NMR (400 MHz, CDCl₃) δ 9.24 (s, 1H), 7.85 (d, J=2.8 Hz, 1H), 7.42 (d, J=2.8 Hz, 1H), 7.09-7.03 (m, 1H), 6.98 (d, J=7.2 Hz, 1H), 6.93-6.89 (m, 1H), 6.02 (s, 1H), 4.36 (d, J=17.2 Hz, 1H), 4.08-3.99 (m, 3H), 3.82-3.69 (m, 2H), 3.51-3.44 (m, 1H), 3.35-3.28 (m, 1H), 2.97 (s, 3H), 2.96-2.84 (m, 1H), 2.53 (d, J=1.6 Hz, 3H), 2.01-1.93 (m, 2H), 1.30 (s, 3H), 1.27 (s, 3H), 1.11 (t, J=7.2 Hz, 3H).

### Compound 1: 3-(4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylpropanoic acid

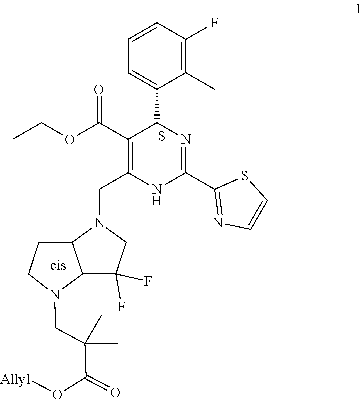

Compound 1 was prepared analogous to compound 1A. Purified by C18 column (acetonitrle:water=1:20 to 2:1) to give the desired product (6.5 mg, 94% purity, 6% yield) as yellow solids. LC-MS (ESI): mass calcd. for C₂₉H₃₄F₃N₅O₄S 605.2, m/z found 606.3 [M+H]⁺. ¹H NMR (400 MHz, CDCl₃) δ 12.65 (br s, 1H), 9.27 (d, J=33.6 Hz, 1H), 7.84 (dd, J=6.4, 2.8 Hz, 1H), 7.41 (d, J=2.8 Hz, 1H), 7.11-7.04 (m, 1H), 6.97-6.91 (m, 2H), 6.00 (d, J=8.4 Hz, 1H), 4.38-3.99 (m, 4H), 3.81-3.67 (m, 2H), 3.55-3.26 (m, 2H), 3.05-2.84 (m, 4H), 2.54 (t, J=2.4 Hz, 3H), 2.01-1.93 (m, 1H), 1.91-1.83 (m, 1H), 1.29 (s, 3H), 1.26 (s, 3H), 1.11 (q, J=2.4 Hz, 3H).

### Compound 2: (cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorooctahydrocyclo-penta[b]pyrrole-5-carboxylic acid

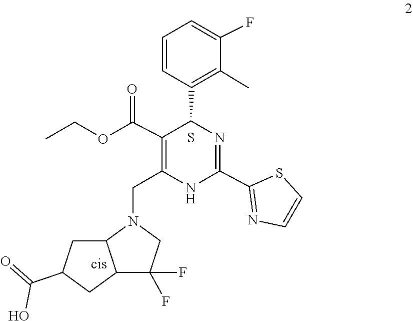

### Preparation of Intermediate S2

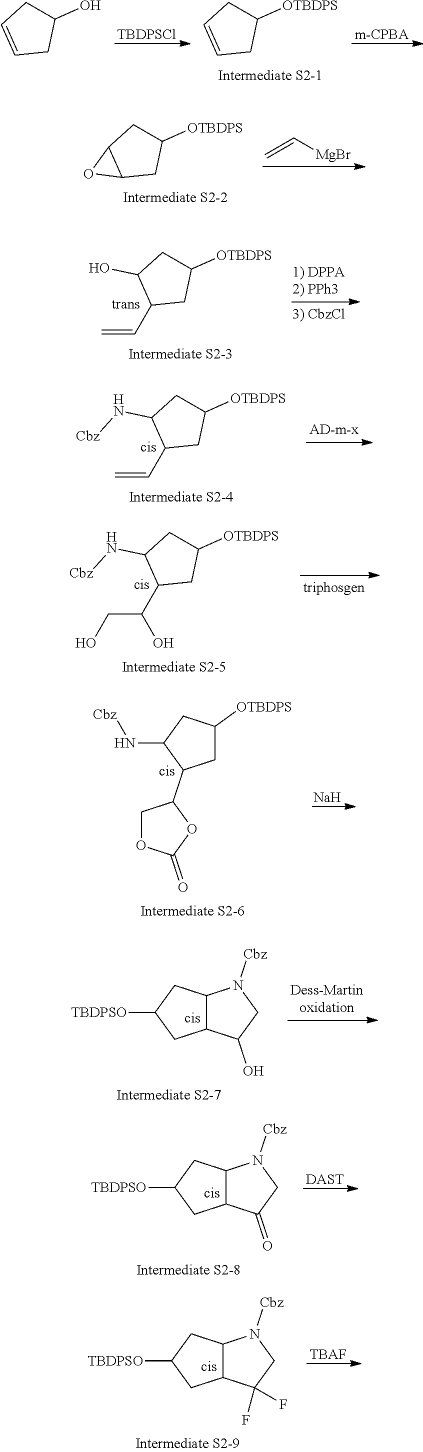

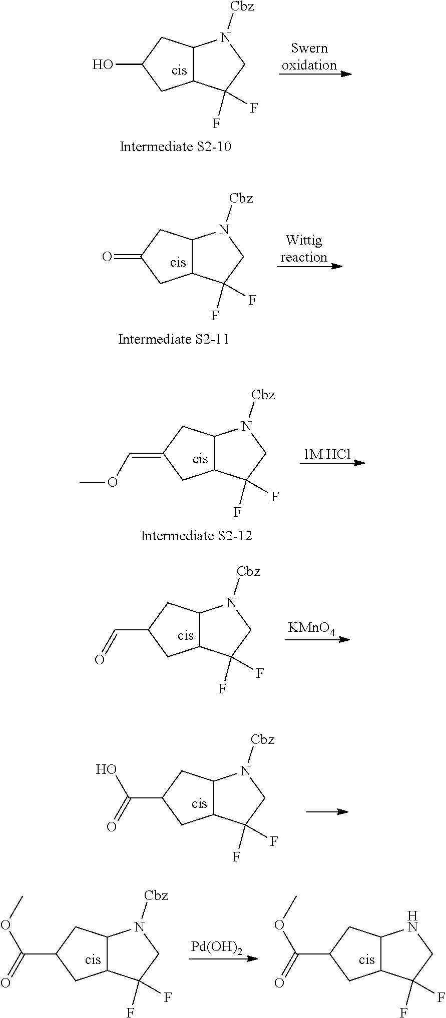

### S2-1: tert-butyl(cyclopent-3-en-1-yloxy)diphenylsilane

To a solution of cyclopent-3-enol (15 g, 178 mmol) and imidazole (36.4 g, 535 mmol) in dichloromethane (300 mL) was added tert-butylchlorodiphenylsilane (51.5 g, 187 mmol) 0° C. After stirred at 20° C. for 3 hours, the reaction mixture was concentrated and purified by silica gel column chromatography (petroleum ether) to give the title compound (50 g, 87% yield) as colorless oil. ¹H NMR (400 MHz, CDCl₃) δ 7.67-7.66 (m, 4H), 7.42-7.21 (m, 6H), 5.60 (s, 2H), 4.58-4.53 (m, 1H), 2.45-2.35 (m, 4H), 1.05 (s, 9H).

### S2-2: (6-oxabicyclo[3.1.0]hexan-3-yloxy)(tert-butyl)diphenylsilane

To a solution of tert-butyl(cyclopent-3-en-1-yloxy)diphenylsilane S2-1 (60 g, 186 mmol) in dichloromethane (500 mL) was added 3-chloroperoxybenzoic acid (41.5 g, 85% purity, 204 mmol). After stirred at 20° C. for 14 hours, the reaction mixture was quenched with saturated sodium sulfite (500 mL). The organic layer was separated and the aqueous layer was extracted with dichloromethane (500 mL). The combined organic layers were dried over Na₂SO₄₍ₛ₎ and filtered. The filtrate was concentrated and purified by silica gel column chromatography (petreloum ether:ethyl acetate=8:1 to 2:1) to give the title compound (61 g, 97% yield) as colorless oil. ¹H NMR (400 MHz, CDCl₃) δ 7.67-7.61 (m, 4H), 7.42-7.34 (m, 6H), 4.43-4.39 (m, 0.4H), 4.11-4.06 (m, 0.6H), 3.41 (s, 0.8H), 3.38 (s, 1.2H), 2.28-2.23 (m, 1H), 2.05-2.01 (m, 1H), 1.87-1.82 (m, 1H), 1.72-1.64 (m, 1H), 1.05 (s, 3.6), 1.04 (s, 5.4H).

### S2-3: (trans)-4-((tert-butyldiphenylsilyl)oxy)-2-vinylcyclopentanol

To a suspension of cuprous iodide (2.28 g, 12.0 mmol) in tetrahydrofuran (200 mL) was added 1 M vinylmagnesium bromide in tetrahydrofuran (120 mL, 120 mmol) at −40° C. The resulting mixture was stirred at −40° C. for 1 hour before (6-oxabicyclo[3.1.0]hexan-3-yloxy)(tert-butyl)diphenylsilane S2-2 (33.8 g, 100 mmol) was added at the same temperature. After stirred at −40° C. for another 1 hour, the mixture was warmed to 15° C. and stirred at 15° C. for 14 hours. The reaction was quenched with saturated ammonium chloride (1000 mL) and extracted with dichloromethane (500 mL) twice. The combined organic layers were washed with brine (500 mL), dried over Na₂SO₄₍ₛ₎ and filtered. The filtrate was concentrated and purified by silica gel column chromatography (petroleum ether:ethyl acetate=15:1) to give the title compound (27 g, 74% yield) as colorless oil. ¹H NMR (400 MHz, CDCl₃) δ 7.70-7.67 (m, 4H), 7.47-7.37 (m, 6H), 4.91-4.81 (m, 0.5H), 5.75-5.67 (m, 0.5H), 5.18-4.98 (m, 2H), 4.39-4.35 (m, 1H), 3.87-3.80 (m, 0.5H), 2.92-2.84 (m, 0.5H), 2.22-2.10 (m, 2H), 1.99-1.47 (m, 3H), 1.10-1.07 (m, 9H).

### S2-4: benzyl ((cis)-4-((tert-butyldiphenylsilyl)oxy)-2-vinylcyclopentyl)carbamate

To a solution of (trans)-4-((tert-butyldiphenylsilyl)oxy)-2-vinylcyclopentanol S2-3 (27 g, 73.6 mmol), triphenylphosphine (21.2 g, 80.9 mmol) and diisopropyl azodicarboxylate (17.8 g, 88.1 mmol) in tetrahydrofuran (300 mL) was added diphenylphosphoryl azide (28.4 g, 103 mmol) dropwise at 0° C. The mixture was stirred at room temperature for 3 hours, before water (50 mL) was added. After triphenylphosphine (32 g, 122 mmol) was added at 45° C., the mixture was stirred at 50° C. for 14 hours. The mixture was cooled down to 0° C., and then saturated sodium bicarbonate aqueous solution (100 mL) was added followed with benzyl carbonochloridate (30 mL, 213 mmol). After stirred at room temperature for 2 hours, the reaction mixture was poured into water (300 mL) and extracted with ethyl acetate (200 mL) twice. The combined organic layers were washed with brine (500 mL), dried over Na₂SO₄₍ₛ₎ and filtered. The filtrate was concentrated and purified by chromatography on silica gel (petroleum ether:ethyl acetate=10:1) to give the title compound (20 g, 90% purity from ¹H NMR, 49% yield) as colorless oil. ¹H NMR (400 MHz, CDCl₃) δ 7.71-7.63 (m, 4H), 7.43-7.29 (m, 11H), 6.01-5.87 (m, 0.4H), 5.74-5.57 (m, 0.6H), 5.16-4.95 (m, 4H), 4.40-4.34 (m, 1.5H), 4.16-4.10 (m, 0.5H), 3.13-3.06 (m, 0.4H), 2.59-2.49 (m, 0.6H), 2.17-1.86 (m, 2H), 1.74-1.55 (m, 2H), 1.06 (s, 5.4H), 1.05 (s, 3.6H).

### S2-5: benzyl ((cis)-4-((tert-butyldiphenylsilyl)oxy)-2-(1,2-dihydroxyethyl)cyclopentyl)carbamate

The mixture of AD-mix-beta (20 g, 25.7 mmol) and methanesulfonamide (600 mg, 6.31 mmol) in tert-butanol (120 mL) and water (120 mL) was stirred at 25° C. for 1 hour before benzyl ((cis)-4-((tert-butyldiphenylsilyl)oxy)-2-vinylcyclopentyl) carbamate S2-4 (8 g, 90% purity, 14.4 mmol) was added. After stirred at room temperature for 60 hours, the mixture was diluted with methanol (200 mL) and filtered. The filtrate was concentrated under reduced pressure to remove the volatile. The residue was diluted with ethyl acetate (200 mL), washed with water (100 mL) for 3 times and brine (100 mL), dried over Na₂SO₄₍ₛ₎ and filtered. The filtrate was concentrated and purified by silica gel column chromatography (petroleum ether:ethyl acetate=1:1) to give the title compound (4.0 g, 95% purity from LC-MS, 49% yield) as yellow oil. LC-MS (ESI): mass calcd. for C₃₁H₃₉NO₅Si 533.3, m/z found 534.5 [M+H]⁺.

### S2-6: benzyl ((cis)-4-((tert-butyldiphenylsilyl)oxy)-2-(2-oxo-1,3-dioxolan-4-yl)cyclopentyl)carbamate

To a solution of benzyl ((cis)-4-((tert-butyldiphenylsilyl)oxy)-2-(1,2-dihydroxyethyl) cyclopentyl)carbamate S2-5 (4 g, 95% purity, 7.12 mmol) and triethylamine (2.1 g, 20.8 mmol) in dichloromethane (30 mL) was added triphosgene (1.3 g, 4.38 mmol) at 0° C. After stirred at 0° C. for 1 hour, the mixture was diluted with dichloromethane (100 mL) and water (100 mL). The organic layer was separated and the aqueous layer was extracted with dichloromethane (100 mL). The combined organic layers were washed with water (100 mL) twice and brine (100 mL), dried over Na₂SO₄₍ₛ₎ and filtered. The filtrate was concentrated and purified by silica gel column chromatography (petroleum ether:ethyl acetate=5:1) to give the title compound (3.8 g, 90% purity from ¹H NMR, 86% yield) as yellow oil. ¹H NMR (400 MHz, CDCl₃) δ 7.61-7.59 (m, 4H), 7.44-7.34 (m, 11H), 5.15-5.02 (m, 2H), 4.77-4.63 (m, 1H), 4.57-4.27 (m, 3H), 4.15-4.05 (m, 1H), 2.25-2.07 (m, 1H), 1.99-1.63 (m, 3H), 1.54-1.42 (m, 1H), 1.07-1.04 (m, 9H).

### S2-7: (cis)-benzyl 5-((tert-butyldiphenylsilyl)oxy)-3-hydroxyhexahydrocyclopenta[b]pyrrole-1(2H)-carboxylate

To a solution of benzyl ((cis)-4-((tert-butyldiphenylsilyl)oxy)-2-(2-oxo-1,3-dioxolan-4-yl)cyclopentyl)carbamate S2-6 (3.8 g, 90% purity, 6.11 mmol) in N,N-dimethylformamide (40 mL) was added sodium hydride (2.2 g, 60% wt. in mineral oil, 55.0 mmol) at 0° C. After stirred at room temperature for 1.5 hours, the mixture was diluted with ethyl acetate (100 mL) and poured into ice water (100 mL). The organic layer was separated and the aqueous layer was extracted with ethyl acetate (100 mL). The combined organic layers were washed with water (100 mL) for 3 times and brine (100 mL), dried with Na₂SO₄₍ₛ₎ and filtered. The filtrate was concentrated and purified by silica gel column chromatography (petroleum ether:ethyl acetate=1:1) to give the title compound (2.1 g, 90% purity from ¹H NMR, 60% yield) as yellow oil. ¹H NMR (400 MHz, CDCl₃) δ 7.63-7.62 (m, 4H), 7.44-7.33 (m, 11H), 5.14-5.04 (m, 2H), 4.42-4.02 (m, 3H), 3.83-3.50 (m, 1H), 3.32-2.95 (m, 1H), 2.42-2.14 (m, 1H), 1.88-1.64 (m, 4H), 1.04-1.01 (m, 9H)

### S2-8: (cis)-benzyl 5-((tert-butyldiphenylsilyl)oxy)-3-oxohexahydrocyclopenta[b]pyrrole-1(2H)-carboxylate

To a solution of (cis)-benzyl 5-((tert-butyldiphenylsilyl)oxy)-3-hydroxyhexahydrocyclopenta[b]pyrrole-1(2H)-carboxylate S2-7 (2.1 g, 90% purity, 3.67 mmol) in dichloromethane (45 mL) was added Dess-Martin periodinane (3.0 g, 7.07 mmol). After stirred at room temperature for 14 hours, the mixture was diluted with ethyl acetate (100 mL), and washed with saturated sodium sulfite aqueous solution (100 mL). The aqueous layer was extracted with ethyl acetate (100 mL). The combined organic layers were washed with water (100 mL) 3 times, and with brine (100 mL), dried with Na₂SO₄₍ₛ₎ and filtered. The filtrate was concentrated and purified by silica gel column chromatography (petroleum ether:ethyl acetate=1:1) to give the title compound (1.9 g, 92% purity from LC-MS, 92% yield) as yellow oil. LC-MS (ESI): mass calcd. for C₃₁H₃₅NO₄Si 513.2, m/z found 514.2 [M+H]⁺.

### S2-9: (cis)-benzyl 5-((tert-butyldiphenylsilyl)oxy)-3,3-difluorohexahydrocyclopenta[b]pyrrole-1(2H)-carboxylate

To a solution of (cis)-benzyl 5-((tert-butyldiphenylsilyl)oxy)-3-oxohexahydro cyclopenta[b]pyrrole-1(2H)-carboxylate S2-8 (1.9 g, 92% purity, 3.40 mmol) in dichloromethane (20 mL) was added diethylaminosulfur trifluoride (2.6 g, 16.1 mmol). After stirred at room temperature for 14 hours, the mixture was diluted with ethyl acetate (100 mL) and poured into water (100 mL). The organic layer was separated and the aqueous layer was extracted with ethyl acetate (100 mL). The combined organic layers were washed with water (100 mL) 3 times, and with brine (100 mL), dried with Na₂SO₄₍ₛ₎ and filtered. The filtrate was concentrated and purified by silica gel column chromatography (petroleum ether:ethyl acetate=5:1) to give the title compound (1.4 g, 90% purity from LC-MS, 69% yield) as yellow oil. LC-MS (ESI): mass calcd. for C₃₁H₃₅F₂NO₃Si 535.2, m/z found 536.3 [M+H]⁺.

### S2-10: (cis)-benzyl 3,3-difluoro-5-hydroxyhexahydrocyclopenta[b]pyrrole-1(2H)-carboxylate

To a solution of (cis)-benzyl 5-((tert-butyldiphenylsilyl)oxy)-3,3-difluorohexahydro cyclopenta[b]pyrrole-1(2H)-carboxylate S2-9 (1.2 g, 90% purity, 2.02 mmol) in tetrahydrofuran (20 mL) was added 1 M tetrabutylammonium fluoride in tetrahydrofuran (4 mL, 4 mmol). After stirred at room temperature for 14 hours, the mixture was concentrated and purified by silica gel column chromatography (petroleum ether:ethyl acetate=2:1) to give the title compound (600 mg, 90% purity from ¹H NMR, 90% yield) as brown oil. ¹H NMR (400 MHz, CDCl₃) δ 7.36-7.32 (m, 5H), 5.16-5.09 (m, 2H), 4.61-4.33 (m, 2H), 4.00-3.89 (m, 1.3H), 3.70-3.60 (m, 0.7H), 3.23-3.10 (m, 0.6H), 3.00-2.86 (m, 0.4H), 2.25-1.90 (m, 4H).

### S2-11: (cis)-benzyl 3,3-difluoro-5-oxohexahydrocyclopenta-[b]pyrrole-1(2H)-carboxylate

To a solution of oxalyl dichloride (320 mg, 2.52 mmol) in dichloromethane (3 mL) was added dimethyl sulfoxide (390 mg, 4.99 mmol) in dichloromethane (2 mL) dropwise at −78° C. The mixture stirred at −78° C. for 30 minutes before a solution of (cis)-benzyl 3,3-difluoro-5-hydroxyhexahydrocyclopenta[b]pyrrole-1(2H)-carboxylate S2-10 (450 mg, 85% purity, 1.29 mmol) in dichloromethane (2 mL) was added dropwise at −78° C. After stirred at −78° C. for 1 hour, triethylamine (850 mg, 8.40 mmol) in dichloromethane (3 mL) was added dropwise at −78° C. The reaction mixture was stirred at −78° C. for 30 minutes and then warmed to room temperature. After stirred at room temperature for 1 hour, the reaction mixture was quenched with water (20 mL) and extracted with ethyl acetate (20 mL) three times. The combined organic layers were washed with 0.5 M HCl (10 mL), saturated sodium bicarbonate aqueous solution (10 mL) and brine (10 mL), dried over Na₂SO₄₍ₛ₎ and filtered. The filtrate was concentrated and purified by silica gel column chromatography (petroleum ether:ethyl acetate=4:1) to give the title compound (350 mg, 94% purity from LC-MS, 87% yield) as yellow oil. LC-MS (ESI): mass calcd. for C₁₅H₁₅F₂NO₃ 295.1, m/z found 296.2 [M+H]⁺. ¹H NMR (400 MHz, CDCl₃) δ 7.39-7.36 (m, 5H), 5.18-5.11 (m, 2H), 4.64 (s, 1H), 3.97-3.85 (m, 2H), 3.27-3.19 (m, 1H), 2.78-2.52 (m, 4H).

### S2-12: (cis)-benzyl 3,3-difluoro-5-(methoxymethylene)hexahydrocyclopenta[b]pyrrole-1(2H)-carboxylate

To a solution of (cis)-benzyl 3,3-difluoro-5-oxohexahydrocyclopenta[b]pyrrole-1(2H)-carboxylate S2-11 (350 mg, 94% purity, 1.11 mmol) and dimethyl (1-diazo-2-oxopropyl)phosphonate (384 mg, 2.00 mmol) in methanol (6 mL) was added potassium carbonate (307 mg, 2.22 mmol) slowly at 0° C. After stirred at 0° C. for 30 minutes, the reaction was warmed to room temperature and stirred at room temperature for 2 hours. The mixture was quenched with water (10 mL) and extracted with ethyl acetate (10 mL) three times. The combined organic layers were washed with brine (10 mL), dried over Na₂SO₄₍ₛ₎ and filtered. The filtrate was concentrated and purified by silica gel column chromatography (petroleum ether:ethyl acetate=10:1) to give the title compound (65 mg, 80% purity from LC-MS, 14% yield) as yellow oil. LC-MS (ESI): mass calcd. for C₁₇H₁₉F₂NO₃ 323.1, m/z found 324.0 [M+H]⁺.

### S2-13: (cis)-benzyl 3,3-difluoro-5-formylhexahydrocyclopenta[b]-pyrrole-1(2H)-carboxylate

To a solution of (cis)-benzyl 3,3-difluoro-5-(methoxymethylene)hexahydro-cyclopenta[b]pyrrole-1(2H)-carboxylate S2-12 (65 mg, 80% purity, 0.161 mmol) in acetonitrile (1 mL) was added 1 M HCl (0.3 mL) at 0° C. After stirred at room temperature for 3 hours, the mixture was diluted with brine (2 mL) and basified with saturated sodium bicarbonate aqueous solution to pH˜8, extracted with ethyl acetate (5 mL) three times. The combined organic layers were dried over Na₂SO₄₍ₛ₎ and filtered. The filtrate was concentrated under reduced pressure to give the title compound (63 mg, 76% purity from LC-MS, 96% yield) as brown oil. LC-MS (ESI): mass calcd. for C₁₆H₁₇F₂NO₃ 309.1, m/z found 310.2 [M+H]⁺.

### S2-14: (cis)-1-((benzyloxy)carbonyl)-3,3-difluorooctahydro-cyclopenta[b]pyrrole-5-carboxylic acid

To a solution of (cis)-benzyl 3,3-difluoro-5-formylhexahydrocyclopenta[b]pyrrole-1(2H)-carboxylate S2-13 (63 mg, 76% purity, 0.155 mmol) in acetone (2 mL) and water (0.7 mL) was added potassium permanganate (60 mg, 0.380 mmol) at 0° C. The resulting mixture was stirred at 0° C. for 10 minutes and then at 25° C. for 1 hour. Sodium bisulfite (100 mg, 0.961 mmol) was added, then the mixture was diluted with acetone (2 mL) and water (2 mL). The resulted suspension was stirred at 25° C. for 15 minutes and filtered through Celite®. The filtrate was concentrated under reduced pressure to remove acetone. The resulting aqueous solution was acidified with 0.5 M hydrochloric acid aqueous solution (0.1 mL) to pH˜3 and extracted with ethyl acetate (10 mL) three times. The combined organic layers were dried over Na₂SO₄₍ₛ₎ and filtered. The filtrate was concentrated to give the title compound (56 mg, 53% purity from LC-MS, 59% yield) as yellow oil. LC-MS (ESI): mass calcd. for C₁₆H₁₇F₂NO₄ 325.1, m/z found 326.3 [M+H]⁺.

### S2-15: (cis)-1-benzyl 5-methyl 3,3-difluorohexahydro-cyclopenta[b]pyrrole-1,5(2H)-dicarboxylate

To a solution of (cis)-1-((benzyloxy)carbonyl)-3,3-difluorooctahydro-cyclopenta[b]pyrrole-5-carboxylic acid S2-14 (56 mg, 53% purity, 0.091 mmol) and iodomethane (72 mg, 0.507 mmol) in N,N-dimethylformamide (2 mL) was added potassium carbonate (50 mg, 0.362 mmol). After stirred at room temperature for 16 hours, the mixture was purified by C18 column (acetonitrile:water=20% to 95%) to give the title compound (30 mg, 94% purity from LC-MS, 91% yield) as yellow oil. LC-MS (ESI): mass calcd. for C₁₇H₁₉F₂NO₄ 339.1, m/z found 340.1 [M+H]⁺.

### S-2: (cis)-methyl 3,3-difluorooctahydrocyclopenta[b]pyrrole-5-carboxylate

To a solution of (cis)-1-benzyl 5-methyl 3,3-difluorohexahydrocyclopenta[b]pyrrole-1,5(2H)-dicarboxylate S2-15 (30 mg, 94% purity, 0.083 mmol) in tetrahydrofuran (1 mL) and isopropyl alcohol (1 mL) was added 20% palladium hydroxide on charcoal (30 mg) at room temperature. After stirred at 50° C. under 15 psi hydrogen atmosphere for 2 hours, the mixture was cooled down to room temperature and then filtered. The filtrate was concentrated to give the title compound (25 mg, 62% purity from LC-MS, 91% yield) as yellow oil. LC-MS (ESI): mass calcd. for C₉H₁₃F₂NO₂ 205.1, m/z found 206.2 [M+H]⁺.

### Compound 2-A: (cis)-methyl 1-(((S)-5-(ethoxycarbonyl)-6-(3-fluoro-2-methylphenyl)-2-(thiazol-2-yl)-3,6-dihydropyrimidin-4-yl)methyl)-3,3-difluorooctahydrocyclopenta[b]pyrrole-5-carboxylate

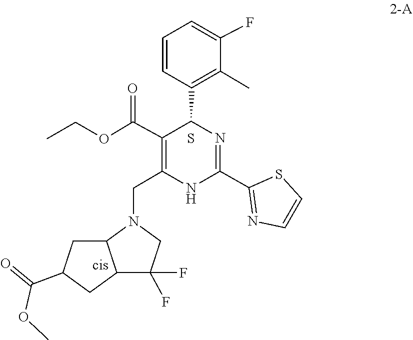

This compound was made according to typical coupling method 1 from H2-1A and S2. LC-MS (ESI): mass calcd. for C₂₇H₂₉F₃N₄O₄S 562.2, m/z found 563.2 [M+H]⁺.

### Compound 2: (cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorooctahydrocyclo-penta[b]pyrrole-5-carboxylic acid

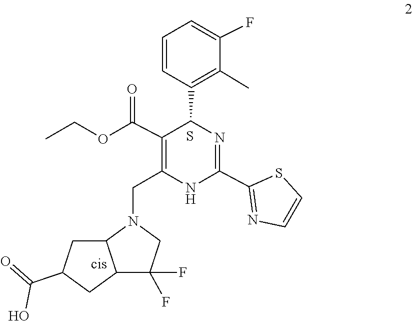

To a solution of (cis)-methyl 1-(((S)-5-(ethoxycarbonyl)-6-(3-fluoro-2-methylphenyl)-2-(thiazol-2-yl)-3,6-dihydropyrimidin-4-yl)methyl)-3,3-difluorooctahydrocyclopenta[b]pyrrole-5-carboxylate compound 2-A (32 mg, 64% purity, 0.036 mmol) in tetrahydrofuran (2 mL), ethanol (1 mL) and water (0.5 mL) was added lithium hydroxide monohydrate (5 mg, 0.119 mmol). After stirred at room temperature under nitrogen atmosphere for 5 hours, the mixture was concentrated under reduced pressure to remove the volatile. The residue was diluted with water (3 mL). The resulting solution was acidified with 1 M HCl (0.1 mL) to pH˜3 and extracted with ethyl acetate (10 mL) three times. The combined organic layers were washed with brine (20 mL), dried over Na₂SO₄₍ₛ₎ and filtered. The filtrate was concentrated and purified by Prep. HPLC (Column: X-bridge C18 (5 um 19*150 mm); Mobile Phase A: water (+0.1% ammonium bicarbonate), Mobile Phase B: acetonitrile, UV: 254 nm, Flow rate: 15 mL/min, Gradient: 20-50% (% B)) to give the title compound (15 mg, 98.9% purity, 74% yield) as yellow solid. LC-MS (ESI): mass calcd. for C₂₆H₂₇F₃N₄O₄S 548.2, m/z found 549.2 [M+H]⁺. ¹H NMR (400 MHz, CD₃OD) δ 7.33-7.92 (m, 1H), 7.73-7.70 (m, 1H), 7.19-7.08 (m, 2H), 6.97-6.92 (m, 1H), 5.99-5.97 (m, 1H), 4.34-4.03 (m, 4H), 3.72-3.65 (m, 1H), 3.26-3.85 (m, 4H), 2.54-2.52 (m, 3H), 2.42-2.32 (m, 1H), 2.29-2.03 (m, 2H), 1.94-1.69 (m, 1H), 1.18-1.12 (m, 3H).

### Compound 3: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3a-fluoro-4-oxohexahydropyrrolo[3,4-b]pyrrol-5(1H)-yl)-2,2-dimethylpropanoic acid

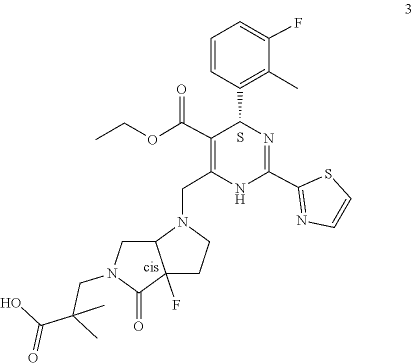

### Preparation of Intermediate S3: 3-((cis)-3a-fluoro-4-oxohexahydropyrrolo[3,4-b]pyrrol-5(1H)-yl)-2,2-dimethylpropanoic acid

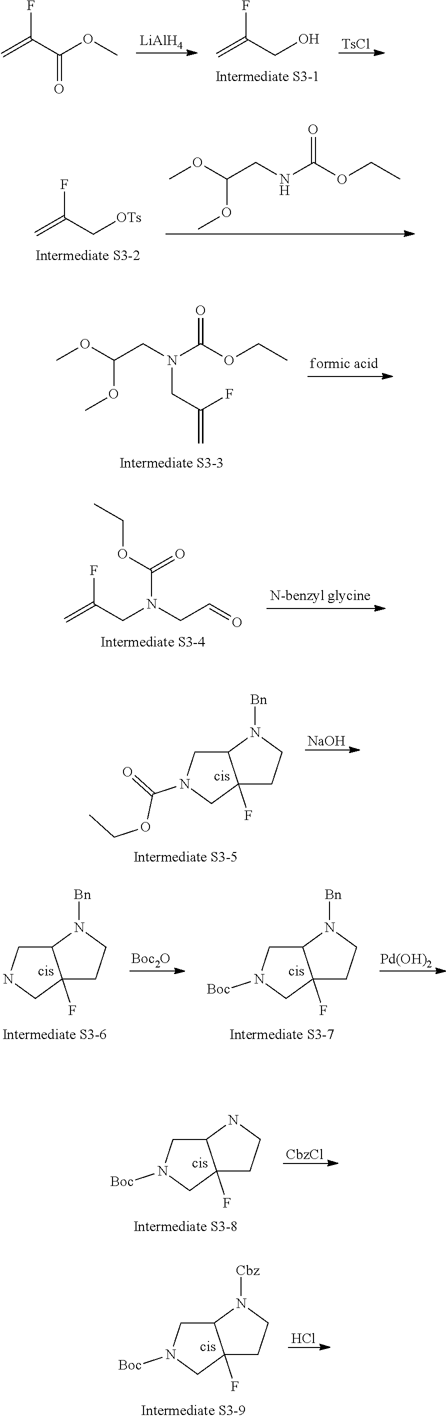

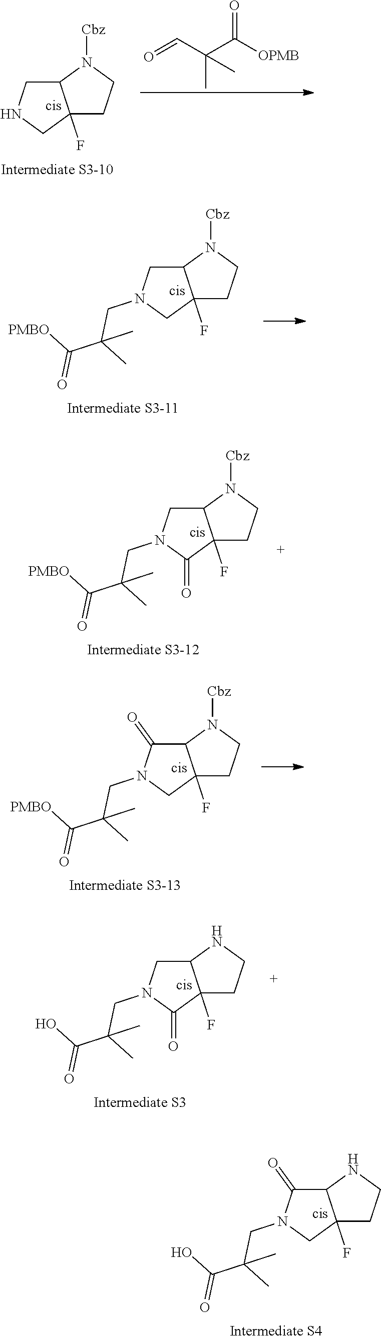

### S3-1: 2-fluoroprop-2-en-1-ol

To a solution of lithium aluminum hydride (7.6 g, 200 mmol) in diethyl ether (150 mL) was treated carefully with aluminum (III) chloride (8.9 g, 66.9 mmol) at −5° C. After stirred at −5° C. for 0.5 hour, methyl 2-fluoroacrylate (14 g, 135 mmol) was added dropwise. After stirred at −5° C. for another 1 hour, an excess of sodium sulfate decahydrate was added to decompose the excess of lithium aluminum tetrahydride. The resulting mixture was filtered and the filtrate as a solution of desired product in diethyl ether (150 mL) was directly used in the subsequent step.

### S3-2: 2-fluoroallyl 4-methylbenzenesulfonate

To the solution of 2-fluoroprop-2-en-1-ol S3-1 in diethyl ether (150 mL) was added tosyl chloride (30.7 g, 161 mmol) and sodium hydroxide (10.7 g, 268 mmol) at room temperature. After stirred at room temperature under nitrogen atmosphere overnight, the mixture was diluted with water (50 mL) and extracted with ethyl acetate (50 mL) three times. The combined organic layers were washed with brine (200 mL), dried over Na₂SO₄₍ₛ₎ and filtered. The filtrate was concentrated and purified by silica gel column chromatography (petroleum ether:ethyl acetate=30:1) to give the title compound (7.6 g, 25% yield) as colorless oil. LC-MS (ESI): mass calcd. for C₁₀H₁₁FO₃S 230.0, m/z found 248.2 [M+NH₄]⁺. ¹H NMR (400 MHz, CDCl₃) δ 7.81 (d, J=8.4 Hz, 2H), 7.36 (d, J=8.0 Hz, 2H), 4.81 (dd, J=15.2 Hz, 3.6 Hz, 1H), 4.64 (dd, J=46.4 Hz, 3.6 Hz, 1H), 4.53 (d, J=14.4 Hz, 2H), 2.46 (s, 3H).

### S3-3: ethyl (2,2-dimethoxyethyl)(2-fluoroallyl)carbamate

To a solution of ethyl (2,2-dimethoxyethyl)carbamate (5.8 g, 32.8 mmol) in toluene (60 mL) was added sodium hydroxide (9.2 g, 230 mmol), benzyltriethylammonium chloride (375 mg, 1.65 mmol), 2-fluoroallyl 4-methylbenzenesulfonate S3-2 (7.6 g, 33 mmol) at room temperature. After stirred at room temperature under nitrogen atmosphere overnight, the mixture was diluted with water (50 mL) and extracted with ethyl acetate (100 mL) three times. The combined organic layers were dried over Na₂SO₄₍ₛ₎ and filtered. The filtrate was concentrated and purified by silica gel column chromatography (petroleum ether:ethyl acetate=30:1) to give the title compound (6.2 g, 80% yield) as a yellow oil. LC-MS (ESI): mass calcd. for C₁₀H₁₈FNO₄ 235.1, m/z found 204.3 [MH−32(CH₃OH)]⁺. ¹H NMR (400 MHz, CDCl₃) δ 4.71-4.66 (m, 1H), 4.49-4.31 (m, 2H), 4.19-4.17 (m, 2H), 4.14-4.03 (m, 2H), 3.40 (s, 8H), 1.28-1.25 (m, 3H).

### S3-4: ethyl (2-fluoroallyl)(2-oxoethyl)carbamate

The mixture of ethyl (2,2-dimethoxyethyl)(2-fluoroallyl)carbamate S3-3 (9.2 g, 39.1 mmol) in 85% formic acid aqueous solution (40 mL) was stirred at room temperature for 12 hours. The mixture was evaporated to dryness under reduced pressure to give a residue, which was dissolved in ethyl acetate (100 mL) and washed with sodium bicarbonate solution (20 mL) and brine (20 mL), dried over Na₂SO₄₍ₛ₎ and filtered. The filtrate was concentrated to give the title compound (7.2 g, 97% yield) as yellow oil. ¹H NMR (400 MHz, CDCl₃) δ 9.59 (d, J=4.0 Hz, 1H), 4.74 (dd, J=12.0 Hz, 4.4 Hz, 1H), 4.49 (dd, J=48.0 Hz, 27.2 Hz, 1H), 4.23-4.02 (m, 6H), 1.26 (dt, J=23.2 Hz, 6.8 Hz, 3H).

### S3-5: (cis)-ethyl 1-benzyl-3a-fluorohexahydropyrrolo[3,4-b]pyrrole-5(1H)-carboxylate

To a solution of ethyl (2-fluoroallyl)(2-oxoethyl)carbamate S3-4 (7.2 g, 38.1 mmol) in toluene (100 mL) was added 2-(benzylamino)acetic acid (7.0 g, 42.4 mmol) at room temperature. After stirred at 120° C. in a Dean-Stark apparatus for 24 hours, the mixture was evaporated to dryness under reduced pressure. The residue was dissolved in ethyl acetate (100 mL) and washed with brine (20 mL), dried over Na₂SO₄₍ₛ₎ and filtered. The filtrate was concentrated and purified by silica gel column chromatography (petroleum ether:ethyl acetate=10:1) to give the title compound (3.3 g, 30% yield) as colorless oil. LC-MS (ESI): mass calcd. for C₁₆H₂₁FN₂O₂ 292.2, m/z found 293.3 [M+H]⁺. ¹H NMR (400 MHz, CDCl₃) δ 7.33-7.28 (m, 5H), 4.13 (q, J=7.2 Hz, 2H), 3.86-3.67 (m, 3H), 3.58-3.46 (m, 3H), 3.20-3.12 (m, 1H), 3.00 (t, J=8.4 Hz, 1H), 2.63-2.56 (m, 1H), 2.21-2.01 (m, 2H), 1.26 (t, J=7.2 Hz, 3H).

### S3-6: (cis)-1-benzyl-3a-fluorooctahydropyrrolo[3,4-b]pyrrole

To a solution of (cis)-ethyl 1-benzyl-3a-fluorohexahydropyrrolo[3,4-b] pyrrole-5(1H)-carboxylate S3-5 (3.1 g, 10.6 mmol) in ethanol (40 mL) and water (10 mL) was added sodium hydroxide (2.1 g, 52.5 mmol) under nitrogen atmosphere. After stirred at 80° C. under nitrogen atmosphere for 14 hours, the mixture was concentrated under the reduced pressure. The aqueous layer was extracted with dichloromethane (20 mL) three times, dried over Na₂SO₄₍ₛ₎ and filtered. The filtrate was concentrated to give the title compound (2.0 g, 85% yield) as yellow oil. LC-MS (ESI): mass calcd. for C₁₃H₁₇FN₂ 220.1, m/z found 221.1 [M+H]⁺. ¹H NMR (400 MHz, CDCl₃) δ 7.33-7.24 (m, 5H), 3.78 (d, J=13.2 Hz, 1H), 3.52 (d, J=12.8 Hz, 1H), 3.08-2.92 (m, 3H), 2.87-2.82 (m, 1H), 2.71 (s, 0.6H), 2.68 (s, 0.4H), 2.59-2.46 (m, 1H), 2.26-2.14 (m, 1H), 1.97-1.80 (m, 3H).

### S3-7: (cis)-tert-butyl 1-benzyl-3a-fluorohexahydropyrrolo[3,4-b]pyrrole-5(1H)-carboxylate

To a solution of (cis)-1-benzyl-3a-fluorooctahydropyrrolo[3,4-b]pyrrole S3-6 (1.0 g, 4.54 mmol) in dichloromethane (30 mL) was added triethylamine (1.4 g, 13.9 mmol) and di-tert-butyl dicarbonate (1.2 g, 5.50 mmol) at room temperature. After stirred at room temperature for 12 hours, the mixture was diluted with dichloromethane (20 mL), washed with 1 M HCl (10 mL) and brine (50 mL), dried over Na₂SO₄₍ₛ₎ and filtered. The filtrate was concentrated and purified by silica gel column chromatography (petroleum ether:ethyl acetate=20:1) to give the title compound (1.2 g, 83% yield) as colorless oil. LC-MS (ESI): mass calcd. for C₁₈H₂₅FN₂O₂ 320.2, m/z found 321.2 [M+H]⁺. ¹H NMR (400 MHz, CDCl₃) δ 7.32-7.29 (m, 5H), 3.83 (d, J=13.6 Hz, 1H), 3.74-3.64 (m, 2H), 3.51-3.32 (m, 3H), 3.14 (dd, J=24.0 Hz, 3.6 Hz, 1H), 3.02-2.98 (m, 1H), 2.63-2.57 (m, 1H), 2.23-2.06 (m, 2H), 1.45 (s, 9H).

### S3-8: (cis)-tert-butyl 3a-fluorohexahydropyrrolo[3,4-b]pyrrole-5(1H)-carboxylate

To a solution of (cis)-tert-butyl 1-benzyl-3a-fluorohexahydropyrrolo[3,4-b]pyrrole-5(1H)-carboxylate S3-7 (1.46 g, 4.56 mmol) in isopropyl alcohol (20 mL) was added 20% palladium hydroxide on charcoal (700 mg) under nitrogen atmosphere. After stirred at 40° C. under hydrogen atmosphere (15 psi) overnight, the mixture was cooled to room temperature and filtered. The filtrate was concentrated to give the title compound (1.0 g, 95% yield) as colorless oil. LC-MS (ESI): mass calcd. for C₁₁H₁₉FN₂O₂ 230.1, m/z found 231.1[M+H]⁺. ¹H NMR (400 MHz, CDCl₃) δ 3.80-3.68 (m, 4H), 3.32-3.29 (m, 1H), 3.24-3.12 (m, 2H), 2.32-2.19 (m, 1H), 2.12-2.02 (m, 1H), 1.45 (s, 9H).

### S3-9: (cis)-1-benzyl 5-tert-butyl 3a-fluorohexahydropyrrolo[3,4-b]pyrrole-1,5-dicarboxylate

To a solution of (cis)-tert-butyl 3a-fluorohexahydropyrrolo[3,4-b]pyrrole-5(1H)-carboxylate S3-8 (700 mg, 3.04 mmol) and sodium bicarbonate (2.5 g, 30 mmol) in tetrahydrofuran (5 mL) and water (5 mL) was added benzyl chloroformate (776 mg, 4.55 mmol) at 0° C. After stirred at room temperature overnight, the mixture was diluted with water (20 mL) and extracted with ethyl acetate (30 mL) for three times. The combined organic layers were washed with brine (20 mL), dried over Na₂SO₄₍ₛ₎ and filtered. The filtrate was concentrated to give a residue, which was purified by silica gel column chromatography (petroleum ether:ethyl acetate=10:1 to 5:1) to give the title compound (1.06 g, 96% yield) as colorless oil. ¹H NMR (400 MHz, CDCl₃) δ 7.36-7.35 (m, 5H), 5.17-5.11 (m, 2H), 4.34-4.24 (m, 1H), 3.91-3.63 (m, 5H), 3.48-3.35 (m, 1H), 2.38-2.29 (m, 1H), 2.22-2.11 (m, 1H), 1.45 (s, 9H).

### S3-10: (cis)-benzyl 3a-fluorohexahydropyrrolo[3,4-b]pyrrole-1(2H)-carboxylate hydrochloride

To a solution of (cis)-1-benzyl 5-tert-butyl 3a-fluorohexahydropyrrolo[3,4-b]pyrrole-1,5-dicarboxylate S3-9 (1.06 g, 2.91 mmol) in ethyl acetate (1 mL) was added 3 M HCl in ethyl acetate (4 mL) under nitrogen atmosphere. After stirred at room temperature under nitrogen atmosphere for 2 hours, the reaction mixture was concentrated to give the title compound (870 mg, 100% yield) as yellow solids. LC-MS (ESI): mass calcd. for C₁₄H₁₈ClFN₂O₂ 300.1, m/z found 265.1 [M−Cl]⁺. ¹H NMR (400 MHz, CDCl₃) δ 10.39-10.29 (m, 2H), 7.38-7.35 (m, 5H), 5.19-5.06 (m, 2H), 4.45-4.41 (d, J=16.4 Hz, 1H), 3.76-3.49 (m, 6H), 2.47-2.38 (m, 2H).

### S3-11: (cis)-benzyl 3a-fluoro-5-(3-((4-methoxybenzyl)oxy)-2,2-dimethyl-3-oxopropyl) hexahydropyrrolo[3,4-b]pyrrole-1(2H)-carboxylate

To a solution of (cis)-benzyl 3a-fluorohexahydropyrrolo[3,4-b]pyrrole-1(2H)-carboxylate hydrochloride S3-10 (870 mg, 2.89 mmol) and 4-methoxybenzyl 2,2-dimethyl-3-oxopropanoate (900 mg, 3.81 mmol) in dichloromethane (20 mL) was added acetic acid (1 mL) and 1 M triisopropoxytitanium(IV) chloride in dichloromethane (8.7 mL, 8.7 mmol) at room temperature. After stirred at room temperature under nitrogen atmosphere for 1 hour, sodium triacetoxyborohydride (1.8 g, 8.49 mmol) was added at room temperature. After stirred at room temperature for 16 hours, the mixture was quenched with ice water (10 mL) and concentrated under reduced pressure to remove dichloromethane. The residue was diluted with water (20 mL) and extracted with ethyl acetate (30 mL) for three times. The combined organic layers were dried over Na₂SO₄₍ₛ₎ and filtered. The filtrate was concentrated to give a residue, which was purified by silica gel column chromatography (petroleum ether:ethyl acetate=10:1) to give the title compound (1.2 g, 86% yield) as colorless oil. LC-MS (ESI): mass calcd. for C₂₇H₃₃FN₂O₅ 484.2, m/z found 485.4 [M+H]⁺. ¹H NMR (400 MHz, CDCl₃) δ 7.38-7.24 (m, 7H), 6.87 (d, J=8.0 Hz, 2H), 5.16-5.08 (m, 2H), 5.04-4.98 (m, 2H), 4.13-4.04 (m, 1H), 3.79 (s, 3H), 3.76-3.61 (m, 1H), 3.52-3.46 (m, 1H), 2.93-2.78 (m, 2H), 2.76-2.59 (m, 3H), 2.52-2.48 (m, 1H), 2.33-2.15 (m, 1H), 2.11-1.99 (m, 1H), 1.15 (s, 6H).

### Intermediates S3-12 and S3-13

### (cis)-benzyl 3a-fluoro-5-(3-((4-methoxybenzyl)oxy)-2,2-dimethyl-3-oxopropyl)-4-oxohexahydropyrrolo[3,4-b]pyrrole-1(2H)-carboxylate and

### (cis)-benzyl 3a-fluoro-5-(3-((4-methoxybenzyl)oxy)-2,2-dimethyl-3-oxopropyl)-6-oxohexahydropyrrolo[3,4-b]pyrrole-1(2H)-carboxylate

To a solution of (cis)-benzyl 3a-fluoro-5-(3-((4-methoxybenzyl)oxy)-2,2-dimethyl-3-oxopropyl)hexahydropyrrolo[3,4-b]pyrrole-1(2H)-carboxylate S3-11 (1.0 g, 2.06 mmol) in carbon tetrachloride (20 mL) was added a solution of ruthenium chloride trihydrate (108 mg, 0.413 mmol) and sodium periodate (2.1 g, 9.81 mmol) in water (20 mL) at 0° C. under nitrogen atmosphere. After stirred at room temperature for 1.5 hours, the mixture was filtered. The filtrate was diluted with water (20 mL) and extracted with dichloromethane (30 mL) twice. The combined organic layers were washed with brine (10 mL), dried over Na₂SO₄₍ₛ₎ and filtered. The filtrate was concentrated and purified by silica gel column chromatography (petroleum ether:ethyl acetate=3:1) to give S3-12 (300 mg, 30% yield) and S3-13 (400 mg, 40% yield) as colorless oils.

S3-12: LC-MS (ESI): mass calcd. for C₂₇H₃₁FN₂O₆ 498.2, m/z found 499.4 [M+H]⁺. ¹H NMR (400 MHz, CDCl₃) δ 7.39-7.19 (m, 7H), 6.90-6.84 (m, 2H), 5.19-5.11 (m, 2H), 5.03 (s, 1H), 4.93 (s, 1H), 4.40-4.32 (m, 1H), 3.85-3.73 (m, 4H), 3.70-3.51 (m, 3H), 3.33 (d, J=14.4 Hz, 1H), 3.26 (d, J=10.8 Hz, 0.5H), 3.13 (d, J=10.8 Hz, 0.5H), 2.45-2.28 (m, 2H), 1.21-1.16 (m, 6H).

S3-13: LC-MS (ESI): mass calcd. for C₂₇H₃₁FN₂O₆ 498.2, m/z found 499.5 [M+H]⁺. ¹H NMR (400 MHz, CDCl₃) δ 7.41-7.30 (m, 7H), 6.90-6.87 (d, J=8.8 Hz, 2H), 5.19 (s, 2H), 5.11-5.04 (m, 2H), 4.75-4.57 (m, 1H), 3.80 (s, 3H), 3.77-3.64 (m, 1H), 3.58-3.51 (m, 2H), 3.78-3.32 (m, 2H), 3.12-3.09 (m, 1H), 2.38-2.27 (m, 1H), 2.02-1.89 (m, 1H), 1.21 (s, 3H), 1.18 (s, 3H).

A racemic of (cis)-benzyl 3a-fluoro-5-(3-((4-methoxybenzyl)oxy)-2,2-dimethyl-3-oxopropyl)-4-oxohexahydropyrrolo[3,4-b]pyrrole-1(2H)-carboxylate S3-12 (400 mg, 0.802 mmol) was separated by chiral Prep. HPLC (separation condition: Column: Chiralpak IC 5 um 20*300 mm; Mobile Phase: CO₂:MeOH=60:40 at 50 g/min; Temp: 30° C.; Wavelength; 230 nm) to give S3-12A (140 mg, 35% yield, 100% ee) as colorless oil and S3-12B (140 mg, 35% yield, 100% ee) as colorless oil.

S3-12A: LC-MS (ESI): mass calcd. for C₂₇H₃₁FN₂O₆ 498.2, m/z found 499.3 [M+H]⁺. Chiral analysis (Column: Chiralpak IC 5 μm 4.6*250 mm; Mobile Phase: CO₂:MeOH=60:40 at 3.0 g/min; Temp: 40° C.; Wavelength: 214 nm, R_{T}=3.60 min).

S3-12B: LC-MS (ESI): mass calcd. for C₂₇H₃₁FN₂O₆ 498.2, m/z found 499.3 [M+H]⁺. Chiral analysis (Column: Chiralpak IC 5 μm 4.6*250 mm; Mobile Phase: CO₂:MeOH=60:40 at 3.0 g/min; Temp: 40° C.; Wavelength: 214 nm, R_{T}=7.36 min).

### S3: 3-((cis)-3a-fluoro-4-oxohexahydropyrrolo[3,4-b]pyrrol-5(1H)-yl)-2,2-dimethylpropanoic acid

To a solution of (cis)-benzyl 3a-fluoro-5-(3-((4-methoxybenzyl)oxy)-2,2-dimethyl-3-oxopropyl)-4-oxohexahydropyrrolo[3,4-b]pyrrole-1(2H)-carboxylate S3-12 (300 mg, 0.602 mmol) in isopropyl alcohol (10 mL) was added 20% palladium hydroxide on charcoal (200 mg) under nitrogen atmosphere. After stirred at 40° C. under hydrogen atmosphere (15 psi) for 3 hours, the mixture was cooled to room temperature and filtered. The filtrate was concentrated to give the title compound (130 mg, 88% yield) as white solids. LC-MS (ESI): mass calcd. for C₁₁H₁₇FN₂O₃ 244.1, m/z found 245.4 [M+H]⁺.

Analogously, S3-12A and S3-12B were converted to S3A and S3B.

### Compound 3: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3a-fluoro-4-oxohexahydropyrrolo[3,4-b]pyrrol-5(1H)-yl)-2,2-dimethylpropanoic acid

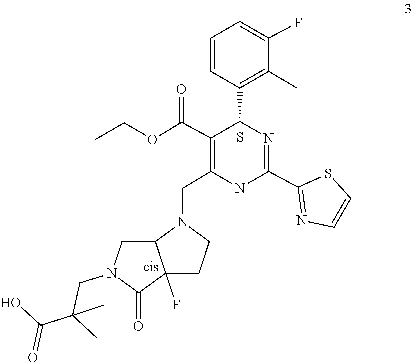

This compound was made according to typical coupling method 1 from H2-1A and S3. Purified by Prep. HPLC (Column: Waters Xbrige C18 (5 μm 19*150 mm), Mobile phase A: water (0.1% ammonium bicarbonate), Mobile phase B: acetonitrile, UV: 214 nm, Flow rate: 15 mL/min, Gradient: 20-45% (% B)) to give the title compound (160 mg, 94.4% purity, 50% yield) as a yellow solid. LC-MS (ESI): mass calcd. for C₂₉H₃₃F₂N₅O₅S 601.2, m/z found 602.3 [M+H]⁺. ¹H NMR (400 MHz, CDCl₃) δ 7.85 (d, J=3.2 Hz, 0.5H), 7.81 (d, J=3.2 Hz, 0.5H), 7.45-7.43 (m, 1H), 7.12-7.06 (m, 1H), 7.03-6.97 (m, 1H), 6.95-6.89 (m, 1H), 6.03 (s, 0.5H), 5.99 (s, 0.5H), 4.56-4.37 (m, 1H), 4.14-3.96 (m, 3H), 3.90-3.78 (m, 1H), 3.65-3.38 (m, 3H), 3.22-3.18 (m, 0.5H), 3.13-3.04 (m, 1H), 3.00-2.98 (m, 0.5H), 2.89-2.85 (m, 0.5H), 2.77-2.70 (m, 0.5H), 2.53-2.52 (m, 3H), 2.46-2.28 (m, 2H), 1.34-1.26 (m, 6H), 1.12 (q, J=7.2 Hz, 3H).

### Compound 3B: 3-(1-(((S)-5-(ethoxycarbonyl)-6-(3-fluoro-2-methylphenyl)-2-(thiazol-2-yl)-3,6-dihydropyrimidin-4-yl)methyl)-3a-fluoro-4-oxohexahydropyrrolo[3,4-b]pyrrol-5(1H)-yl)-2,2-dimethylpropanoic acid (Single Diastereomer)

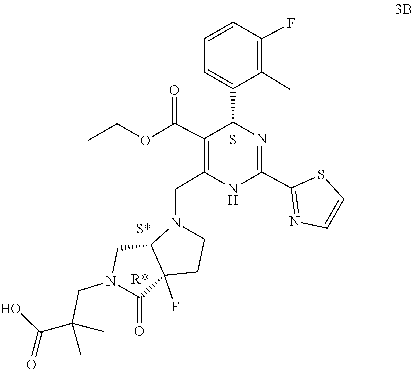

This compound was made according to Typical coupling method 1 from H2-1A with S3B.

Compound 3B: purified by Prep. HPLC (Column: Waters Xbrige C18 (5 μm 19*150 mm), Mobile phase A: water (0.1% ammonium bicarbonate), Mobile phase B: acetonitrile, UV: 214 nm, Flow rate: 15 mL/min, Gradient: 20-45% (% B)) to give the title compound (75 mg, 99.3% purity, 43% yield) as yellow solid. LC-MS (ESI): mass calcd. for C₂₉H₃₃F₂N₅O₅S 601.2, m/z found 602.3 [M+H]⁺. ¹H NMR (400 MHz, CDCl₃) δ 8.82 (s, 1H), 7.86 (d, J=3.2 Hz, 1H), 7.45 (d, J=3.2 Hz, 1H), 7.12-7.06 (m, 1H), 6.99-6.97 (m, 1H), 6.95-6.90 (m, 1H), 5.99 (s, 1H), 4.54 (d, J=16.0 Hz, 1H), 4.14-3.99 (m, 2H), 3.90-3.82 (m, 2H), 3.63-3.58 (m, 1H), 3.55-3.52 (m, 1H), 3.44 (dd, J=24.0 Hz, 5.6 Hz, 1H), 3.23-3.18 (m, 1H), 3.08 (dd, J=13.6 Hz, 1.6 Hz, 1H), 2.90-2.83 (m, 1H), 2.54 (d, J=2.0 Hz, 3H), 2.50-2.31 (m, 2H), 1.31 (s, 3H), 1.28 (s, 3H), 1.13 (t, J=7.2 Hz, 3H).

### Compound 4A: 3-(1-(((S)-5-(ethoxycarbonyl)-6-(3-fluoro-2-methylphenyl)-2-(thiazol-2-yl)-3,6-dihydropyrimidin-4-yl)methyl)-4,6-dioxohexahydropyrrolo[3,4-b]pyrrol-5(1H)-yl)-2,2-dimethylpropanoic acid (Single Diastereomer)

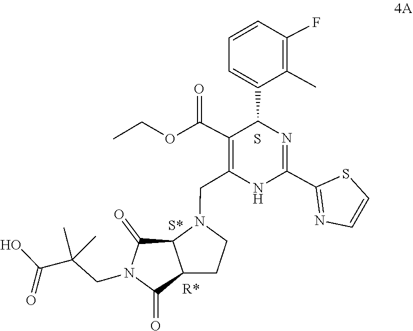

### Preparation of Intermediate S5A and S5B

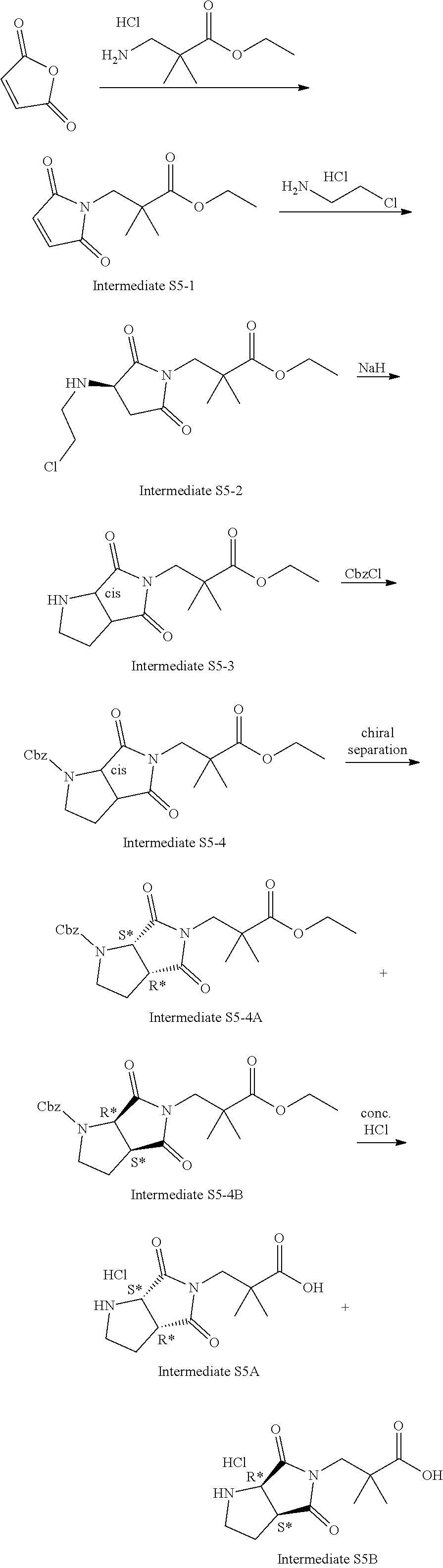

### S5-1: ethyl 3-(2,5-dioxo-2,5-dihydro-1H-pyrrol-1-yl)-2,2-dimethylpropanoate

To a solution of furan-2,5-dione (10 g, 102 mol) in N,N-dimethylformamide (150 mL) were added ethyl 3-amino-2,2-dimethylpropanoate hydrochloride (17.6 g, 96.9 mmol) and triethylamine (9.8 g, 96.8 mmol) at 0° C. under nitrogen atmosphere. The mixture was stirred at the same temperature for 1 hour before sodium acetate (4.2 g, 51.2 mmol) and acetic anhydride (21 g, 206 mmol) was added. After stirred at 60° C. overnight, the reaction mixture was diluted with water (100 mL) and extracted with ethyl acetate (80 mL) three times. The combined organic layers were washed with brine (60 mL), dried over Na₂SO₄₍ₛ₎ and filtered. The filtrate was concentrated and purified by C18 column (acetonitrile:water (+0.1% ammonium bicarbonate)=10% to 80%) to give the title compound (17 g, 90% purity from ¹H NMR, 66% yield) as black oil. ¹H NMR (400 MHz, CDCl₃) δ 6.71 (s, 2H), 4.13 (q, J=7.2 Hz, 2H), 3.67 (s, 2H), 1.27 (t, J=7.2 Hz, 3H), 1.18 (s, 6H).

### S5-2: ethyl 3-(3-((2-chloroethyl)amino)-2,5-dioxopyrrolidin-1-yl)-2,2-dimethylpropanoate

To a solution of ethyl 3-(2,5-dioxo-2,5-dihydro-1H-pyrrol-1-yl)-2,2-dimethylpropanoate S5-1 (3 g, 90% purity, 12.0 mmol) in 1,4-dioxane (40 mL) was added 2-chloroethanamine hydrochloride (1.59 g, 13.7 mmol) and triethylamine (3 g, 29.6 mmol) under nitrogen atmosphere. After stirred at 110° C. overnight, the mixture was cooled down to room temperature, diluted with water (100 mL) and extracted with dichloromethane (90 mL) three times. The combined organic layers were washed with brine (60 mL), dried over Na₂SO₄₍ₛ₎ and filtered. The filtrate was concentrated and purified by C18 column (acetonitrile:water (+0.1% ammonium bicarbonate)=10% to 80%) to give the title compound (2.3 g, 90% purity from ¹H NMR, 56% yield) as brown oil. LC-MS (ESI): mass calcd. for C₁₃H₂₁ClN₂O₄ 304.1, m/z found 305.2 [M+H]⁺. ¹H NMR (400 MHz, CDCl₃) δ 4.15-4.08 (m, 2H), 3.83-3.80 (m, 1H), 3.72-3.63 (m, 4H), 3.13-3.08 (m, 1H), 3.03-2.95 (m, 2H), 2.55-2.49 (m, 1H), 1.28 (t, J=7.6 Hz, 3H), 1.18 (s, 6H).

### S5-3: ethyl 3-((cis)-4,6-dioxohexahydropyrrolo[3,4-b]pyrrol-5(1H)-yl)-2,2-dimethylpropanoate

To a solution of 60% wt. sodium hydride in mineral oil (320 mg, 8.00 mmol) in N,N-dimethylformamide (40 mL) was added ethyl 3-(3-((2-chloroethyl)amino)-2,5-dioxopyrrolidin-1-yl)-2,2-dimethylpropanoate S5-2 (2.3 g, 90% purity, 6.79 mmol) at 0° C. under nitrogen atmosphere. After stirred at room temperature for 1 hour, the mixture was diluted with water (20 mL) and extracted with ethyl acetate (100 mL) three times. The combined organic layers were washed with brine (20 mL), dried over Na₂SO₄₍ₛ₎ and filtered. The filtrate was concentrated to give the title compound (300 mg, 90% purity from ¹H NMR, 15% yield) as yellow solid. LC-MS (ESI): mass calcd. for C₁₃H₂₀N₂O₄ 268.1, m/z found 269.3 [M+H]⁺. ¹H NMR (400 MHz, CDCl₃) δ 4.15-4.08 (m, 3H), 3.68-3.61 (m, 2H), 3.28-3.23 (m, 1H), 3.14-3.10 (m, 1H), 2.76-2.71 (m, 1H), 2.17-2.10 (m, 2H), 1.28-1.26 (m, 3H), 1.18 (s, 6H).

### S5-4: (cis)-benzyl 5-(3-ethoxy-2,2-dimethyl-3-oxopropyl)-4,6-dioxohexahydropyrrolo[3,4-b]pyrrole-1(2H)-carboxylate

To a solution of ethyl 3-((cis)-4,6-dioxohexahydropyrrolo[3,4-b]pyrrol-5(1H)-yl)-2,2-dimethylpropanoate S5-3 (300 mg, 90% purity, 1.01 mmol) in tetrahydrofuran (10 mL) was added benzyl carbonochloridate (0.2 mL, 1.40 mmol) and sodium bicarbonate (100 mg, 1.19 mmol) in water (2 mL) at room temperature. After stirred at room temperature overnight, the mixture was poured into water (50 mL) and extracted with ethyl acetate (50 mL) three times.

The combined organic layers were washed with brine (50 mL), dried over Na₂SO₄₍ₛ₎ and filtered. The filtrate was concentrated and purified by silica gel column chromatography (petroleum ether:ethyl acetate=20:1 to 3:1) to give a crude product, which was further purified by C18 column (acetonitrile:water=5% to 95%) to give the title compound (250 mg, 100% purity from LCMS, 62% yield) as colorless oil. LC-MS (ESI): mass calcd. for C₂₁H₂₆N₂O₆ 402.2, m/z found 403.2 [M+H]⁺.

A racemic mixture of S5-4 (250 mg, 0.621 mmol) was separated by chiral Prep. HPLC (separation condition: Column: Chiralpak IB 5 μm 20*300 mm; Mobile Phase: Hex:EtOH=80:20 at 25 mL/min; Temp: 30° C.; Wavelength: 214 nm) to give S5-4A (85 mg, 90% purity from ¹H NMR, 31% yield, 100% ee) and S5-4B (85 mg, 90% purity from ¹H NMR, 31% yield, 99.9% ee) as yellow solids.

S5-4A: LC-MS (ESI): mass calcd. for C₂₁H₂₆N₂O₆ 402.2, m/z found 403.2 [M+H]⁺. Chiral analysis (Column: Chiralpak IB 5 μm 4.6*250 mm; Mobile Phase: Hex:EtOH=50:50 at 1 mL/min; Temp: 30° C.; Wavelength: 214 nm, R_{T}=5.497 min). ¹H NMR (400 MHz, CDCl₃) δ 7.46-7.31 (m, 5H), 5.21 (s, 2H), 4.98-4.88 (m, 1H), 4.08 (q, J=7.2 Hz, 2H), 4.01-3.91 (m, 1H), 3.65 (s, 2H), 3.39 (t, J=7.6 Hz, 1H), 3.25-3.17 (m, 1H), 2.27-2.13 (m, 2H), 1.25 (t, J=7.2 Hz, 3H), 1.18 (s, 3H), 1.16 (s, 3H).

S5-4B: LC-MS (ESI): mass calcd. for C₂₁H₂₆N₂O₆ 402.2, m/z found 403.2 [M+H]⁺. Chiral analysis (Column: Chiralpak IB 5 μm 4.6*250 mm; Mobile Phase: Hex:EtOH=50:50 at 1 mL/min; Temp: 30° C.; Wavelength: 214 nm, R_{T}=6.875 min). ¹H NMR (400 MHz, CDCl₃) δ 7.45-7.31 (m, 5H), 5.24-5.21 (m, 2H), 4.98-4.89 (m, 1H), 4.08 (q, J=7.2 Hz, 2H), 4.02-3.93 (m, 1H), 3.66 (s, 2H), 3.39 (t, J=7.6 Hz, 1H), 3.25-3.18 (m, 1H), 2.27-2.15 (m, 2H), 1.25 (t, J=7.2 Hz, 3H), 1.18 (s, 3H), 1.16 (s, 3H).

S5A: 3-(4,6-dioxohexahydropyrrolo[3,4-b]pyrrol-5(1H)-yl)-2,2-dimethylpropanoic acid

To a solution of benzyl 5-(3-ethoxy-2,2-dimethyl-3-oxopropyl)-4,6-dioxohexahydropyrrolo[3,4-b]pyrrole-1(2H)-carboxylate S5-4A (85 mg, 90% purity, 0.190 mmol) in 1,4-dioxane (3 mL) and water (5 mL) was added 12 M HCl (5 mL, 60 mmol) at room temperature. After stirred at 80° C. under nitrogen atmosphere for 2 hours, the mixture was concentrated under reduced pressure to give the title compound (55 mg, 90% purity, 94% yield) as white solids. LC-MS (ESI): mass calcd. for C₁₁H₁₇ClN₂O₄ 276.1, m/z found 241.1 [M−Cl]⁺.

Analogously, S5-4B was converted to S5B. LC-MS (ESI): mass calcd. for C₁₁H₁₇ClN₂O₄ 276.1, m/z found 241.1 [M−Cl]⁺.

### Compound 4A: 3-(1-(((S)-5-(ethoxycarbonyl)-6-(3-fluoro-2-methylphenyl)-2-(thiazol-2-yl)-3,6-dihydropyrimidin-4-yl)methyl)-4,6-dioxohexahydropyrrolo[3,4-b]pyrrol-5(1H)-yl)-2,2-dimethylpropanoic acid (Single Diasteromer)

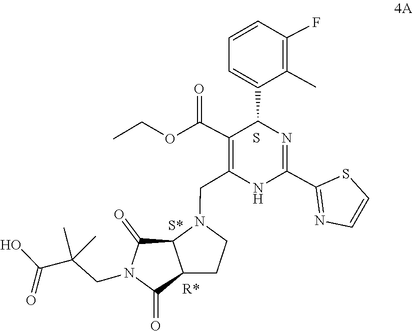

The compound was prepared according to Typical coupling method 1 from H2-1A with S5A.

Compound 4A: LC-MS (ESI): mass calcd. for C₂₉H₃₂FN₅O₆S 597.2, m/z found 598.2 [M+H]⁺. ¹H NMR (400 MHz, DMSO-d₆) δ 9.38 (br s, 1H), 7.98 (s, 0.2H), 7.91 (s, 1.8H), 7.20-7.15 (m, 1H), 7.09-7.01 (m, 2H), 5.88 (s, 0.9H), 5.76 (s, 0.1H), 4.39 (d, J=16.8 Hz, 1H), 4.25 (d, J=16.8 Hz, 1H), 3.99 (q, J=7.2 Hz, 2H), 3.89 (d, J=8.0 Hz, 1H), 3.58-3.43 (m, 3H), 2.83-2.79 (m, 2H), 2.45 (s, 3H), 2.28-2.20 (m, 1H), 1.93-1.89 (m, 1H), 1.07 (t, J=7.2 Hz, 3H), 1.02 (s, 3H), 1.00 (s, 3H).

### Compound 5A: 3-(1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)-2,2-dimethylpropanoic acid

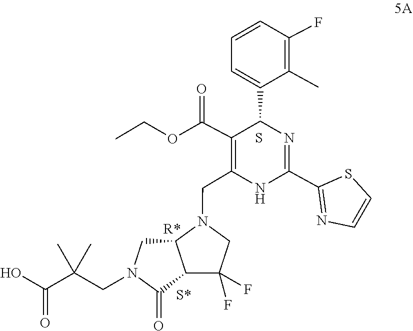

### Preparation of Intermediate S6

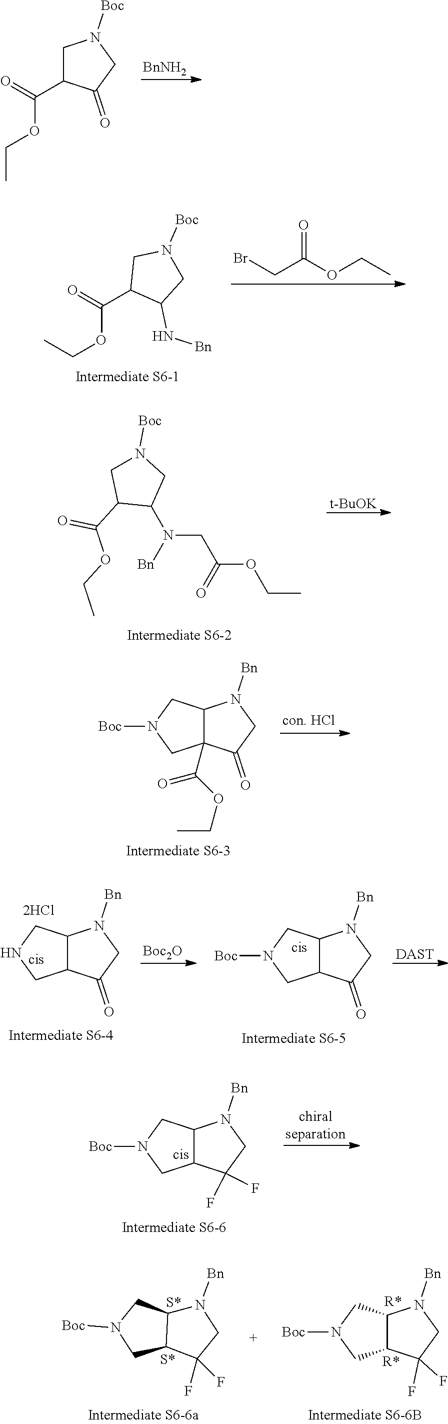

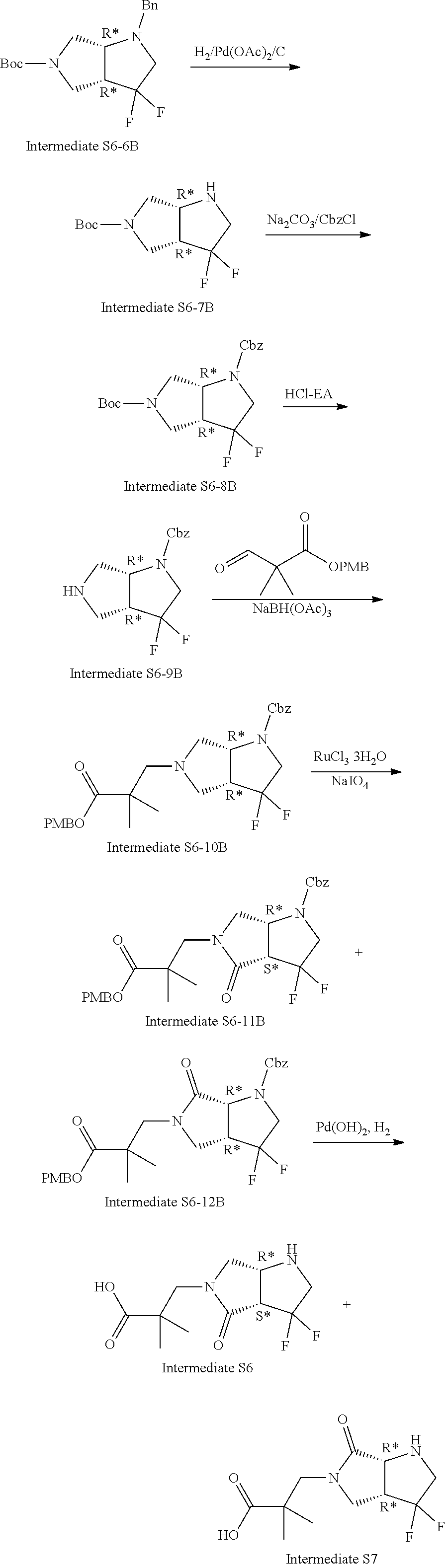

### S6-1: 1-tert-Butyl 3-ethyl 4-(benzylamino)pyrrolidine-1,3-dicarboxylate

To a mixture of ethyl N-Boc-4-oxopyrrolidine-3-carboxylate (20.0 g, 75.4 mmol, 97% purity) in ethanol (300 mL) was added acetic acid (9.0 g, 149.9 mmol) and phenylmethanamine (16.0 g, 149.3 mmol) at room temperature. After addition, the mixture was stirred at room temperature overnight. Sodium cyanotrihydroborate (20.0 g, 318.3 mmol) was added. The mixture was stirred at 75° C. overnight. The reaction mixture was concentrated in vacuum. The residue was poured into water (200 mL) and extracted with ethyl acetate (500 mL) twice. The combined organic phases were washed with brine (200 mL), dried over sodium sulfate and filtered. The filtrate was concentrated in vacuum. The residue was purified by silica gel column chromatography (petroleum ether:ethyl acetate=10:1) to give the desired compound (21.5 g, 73.6% yield, 90% purity from NMR) as a colorless oil. LC-MS (ESI): mass calcd. for C₁₉H₂₈N₂O₄ 348.4, m/z found 349.1 [M+H]⁺. ¹HNMR (400 MHz, CDCl₃): 7.35-7.28 (m, 5H), 4.20-4.14 (m, 2H), 3.83-3.65 (m, 4H), 3.54-3.48 (m, 2H), 3.14-3.12 (m, 1H), 2.94-2.92 (m, 1H), 1.42 (s, 9H), 1.28-1.24 (m, 3H).

### S6-2: 1-tert-Butyl 3-ethyl 4-(benzyl(2-ethoxy-2-oxoethyl)amino)pyrrolidine-1,3-dicarboxylate

To a mixture 1-tert-butyl 3-ethyl 4-(benzylamino)pyrrolidine-1,3-dicarboxylate S6-1 (21.5 g, 55.5 mmol, 90% purity) in acetonitrile (300 mL) were added K₂CO₃ (23.0 g, 166.4 mmol) and ethyl bromoacetate (28.0 g, 167.7 mmol) at room temperature. After addition, the mixture was stirred at 75° C. overnight. The reaction mixture was filtered and concentrated in vacuum. The residue was purified by silica gel column chromatography (petroleum ether:ethyl acetate=5:1 to 3:1) to give the desired compound (25.0 g, 93.2% yield, 90% purity from LCMS) as a colorless oil. LC-MS (ESI): mass calcd. for C₂₃H₃₄N₂O₆ 434.5, m/z found 435.2 [M+H]⁺.

### S6-3: 5-tert-Butyl 3a-ethyl 1-benzyl-3-oxohexahydropyrrolo[3,4-b]pyrrole-3a,5(1H)-dicarboxylate

To a solution of 1-tert-butyl 3-ethyl 4-(benzyl(2-ethoxy-2-oxoethyl)amino)-pyrrolidine-1,3-dicarboxylate S6-2 (18.0 g, 37.3 mmol) in dry toluene (100 mL) was added potassium tert-butoxide (6.3 g, 56.1 mmol) in portions at 0° C. After addition, the reaction mixture was stirred at 0° C. for 2 hours. The reaction mixture was acidified with 1 N HCl to pH=4 and the organic layer was separated. The aqueous layer was extracted with ethyl acetate (150 mL). The combined organic phases were washed with sat. NaHCO₃ solution (50 mL), brine (50 mL), dried over sodium sulfate and filtered. The filtrate was concentrated in vacuum to give the desired compound (15.0 g, 70% yield, 90% purity from NMR) as a yellow oil. ¹HNMR (400 MHz, CDCl₃): 7.29-7.18 (m, 5H), 4.20-4.16 (m, 2H), 4.03-3.86 (m, 4H), 3.76-3.59 (m, 4H), 3.47-3.29 (m, 1H), 3.12-3.03 (m, 1H), 1.42 (s, 9H), 1.31-1.24 (m, 3H).

### S6-4: 1-Benzylhexahydropyrrolo[3,4-b]pyrrol-3(2H)-one

A mixture of cis-5-tert-butyl 3a-ethyl 1-benzyl-3-oxohexahydropyrrolo[3,4-b]pyrrole-3a,5(1H)-dicarboxylate S6-3 (9.0 g, 19.7 mmol, 85% purity) and 12N Hydrochloric acid (150 mL) was stirred at 100° C. for 48 hours. The reaction mixture was concentrated in vacuum to give the desired compound (4.8 g, 85% yield, 87.6% purity from LCMS) as a yellow solid. LC-MS (ESI): mass calcd. for C₁₃H₁₆N₂O 216.3, m/z found 217.1[M+H]⁺.

### S6-5: tert-Butyl 1-benzyl-3-oxohexahydropyrrolo[3,4-b]pyrrole-5(1H)-carboxylate (6)

To a solution of 1-benzylhexahydropyrrolo[3,4-b]pyrrol-3(2H)-one dihydrochloride S6-4 (4.8 g, 16.7 mmol, 87% purity) in dichloromethane (50 mL) was added triethylamine (12.1 g, 119.6 mmol) and tert-butyldicarbonate (5.5 g, 25.2 mmol) at room temperature. After addition, the reaction mixture was stirred at 25° C. overnight. The reaction mixture was concentrated in vacuum. The residue was purified by silica gel column chromatography (petroleum ether:ethyl acetate=6:1) to give the desired compound (3.7 g, 70% yield, 85% purity from LC-MS) as a colorless oil. LC-MS (ESI): mass calcd. for C₁₈H₂₄N₂O₃ 316.4, m/z found 317.1[M+H]⁺.

### S6-6: tert-Butyl 1-benzyl-3,3-difluorohexahydropyrrolo[3,4-b]pyrrole-5(1H)-carboxylate

To a solution of tert-butyl 1-benzyl-3-oxohexahydropyrrolo[3,4-b]pyrrole-5(1H)-carboxylate S6-5 (1.1 g, 3.13 mmol, 90% purity) in dry dichloromethane (40 mL) was added diethylaminosulfur trifluoride (2.6 g, 16.13 mmol, in dry dichloromethane (20 mL)) dropwise at −78° C. After addition, the reaction mixture was stirred at −78° C. for 2 hours, and then warmed to room temperature overnight. The reaction mixture was quenched with sat. NaHCO₃ (10 mL) at 0° C. to pH=7-8 and then extracted with ethyl acetate (100 mL) twice. The combined organic phases were washed with brine (30 mL), dried over sodium sulfate and filtered. The filtrate was concentrated in vacuum. The residue was purified by silica gel column chromatography (petroleum ether:ethyl acetate=10:1) to give the desired compound (760 mg, 68.9% yield, 96% purity from LC-MS) as a yellow solid. LC-MS (ESI): mass calcd. for C₁₈H₂₄F₂N₂O₂ 338.4, m/z found 339.1[M+H]⁺.

Racemic S6-6 (20.8 g, 55.38 mmol) was separated by chiral Prep. HPLC (separation condition: Column: IG-3.0 cm; Mobile Phase: CO₂:IPA (DEA)=80:20 (0.3) at 60 g/min; temp: 35° C.; Wavelength: 214 nm) to afford (S6-6B) (8.7 g, 41.8% yield, 90% purity, 97% ee) and (S6-6A) (6.8 g, 32.7% yield, 90% purity, 100% ee).

S6-6A: HNMR (300 MHz, CDCl3): 7.42-7.26 (m, 5H), 4.05-4.02 (m, 1H), 3.92-3.23 (m, 7H), 3.11-2.95 (m, 1H), 2.91-2.74 (m, 1H), 1.52 (m, 9H).

S6-6B: HNMR (300 MHz, CDCl3): 7.42-7.27 (m, 5H), 4.06-3.93 (m, 1H), 3.89-3.60 (m, 1H), 3.60-3.22 (m, 6H), 3.11-2.94 (m, 1H), 2.90-2.75 (m, 1H), 1.53 (m, 9H).

### S6-7B: tert-Butyl 3,3-difluorohexahydropyrrolo[3,4-b]pyrrole-5(1H)-carboxylate

To a solution of tert-butyl 1-benzyl-3,3-difluoro-hexahydropyrrolo[3,4-b]pyrrole-5(1H)-carboxylate S6-6B (3.00 g, 8.42 mmol, 95% purity) in isopropanol (150 mL) was added palladium acetate (550 mg) and activated carbon (70 mg). The mixture was stirred at 50° C. overnight under hydrogen (50 psi). The reaction mixture was filtered and the filtrate was concentrated under reduced pressure to give the desired product (3.74 g, 94% yield, 49% purity by LC-MS) as yellow oil. The crude was used to next step without purification.

### S6-8B: 1-Benzyl 5-(tert-butyl) 3,3-difluorohexahydro-pyrrolo[3,4-b]pyrrole-1,5-dicarboxylate

To a mixture of tert-butyl 3,3-difluorohexahydropyrrolo[3,4-b]pyrrole-5(1H)-carboxylate S6-7B (3.74 g, 7.38 mmol, 49% purity) in acetonitrile (20 mL) was added benzyl chloroformate (2.52 g, 14.8 mmol), water (20 mL) and sodium carbonate (2.35 g, 22.2 mmol). The mixture was stirred at room temperature overnight. The mixture was poured into water (50 mL) and extracted with ethyl acetate (50 mL) three times. The combined organic layers were washed with brine (100 mL) and dried over anhydrous sodium sulfate (s). The mixture was filtered and the filtrate was concentrated to give a crude product. The crude was purified by C18 column (acetonitrile:water=50% to 70%) to give the desired compound (3.10 g, 99% yield, 90% purity from NMR) as a white solid. ¹H NMR (300 MHz, CDCl₃) 7.43-7.29 (m, 5H), 5.15 (s, 2H), 4.59-4.44 (m, 1H), 4.06-3.91 (m, 1H), 3.80-3.63 (m, 3H), 3.57-3.51 (m, 2H), 3.20-3.04 (m, 1H), 1.45 (s, 9H).

### S6-9B: Benzyl 3,3-difluorohexahydropyrrolo[3,4-b]pyrrole-1(2H)-carboxylate

To a solution of 1-benzyl 5-(tert-butyl) 3,3-difluorohexahydropyrrolo[3,4-b]pyrrole-1,5-dicarboxylate S6-8B (1.50 g, 3.53 mmol, 90% purity) in ethyl acetate (20 mL) was added 3 M hydrogen chloride in ethyl acetate (10 mL, 30 mmol). The mixture was stirred at room temperature for 1 hour. The mixture was concentrated to give a residue. The residue was dissolved in water (30 mL) and basified with saturated sodium bicarbonate aqueous solution to pH 7-8. Then the mixture was extracted with ethyl acetate (30 mL) three times. The combined organic layers were washed with brine (80 mL), dried over anhydrous sodium sulfate and filtered. The filtrate was concentrated to give the title compound (1.30 g, 85% yield, 65% purity by LCMS) as a colorless oil. LC-MS (ESI): mass calcd. for C₁₄H₁₆F₂N₂O₂ 282.12, m/z found 283.2 [M+H]⁺.

### S6-10B: Benzyl 3,3-difluoro-5-(3-((4-methoxybenzyl)oxy)-2,2-dimethyl-3-oxopropyl)-hexahydropyrrolo[3,4-b]pyrrole-1(2H)-carboxylate

To a solution of benzyl 3,3-difluorohexahydropyrrolo[3,4-b]pyrrole-1(2H)-carboxylate S6-9B (1.30 g, 2.99 mmol, 65% purity) and 4-methoxybenzyl 2,2-dimethyl-3-oxopropanoate (800 mg, 3.05 mmol, 90% purity) in dichloromethane (30 mL) was added 1 M triisopropoxytitanium(IV) chloride in hexane (5.0 mL, 5.0 mmol) by dropwise under nitrogen. The mixture was stirred at room temperature for 30 minutes. To the system was added sodium triacetoxyhydroborate (3.20 g, 15.1 mmol) and glacial acetic acid (2 mL). Then the mixture was stirred at room temperature overnight. The reaction mixture was poured into saturated sodium bicarbonate aqueous solution (50 mL) and extracted with ethyl acetate (30 mL) three times. The combined organic layers were washed with brine (80 mL), dried over anhydrous sodium sulfate and filtered. The filtrate was concentrated to give a residue. The residue was purified by C18 column (acetonitrile:water=40% to 65%) to give the title compound (1.30 g, 78% yield, 90% purity by LCMS) as a colorless oil. LC-MS (ESI): mass calcd. for C₂₇H₃₂F₂N₂O₅ 502.2, m/z found 503.2 [M+H]⁺.

### S6-11B and S6-12B: benzyl 3,3-difluoro-5-(3-((4-methoxybenzyl)oxy)-2,2-dimethyl-3-oxopropyl)-4-oxohexahydropyrrolo[3,4-b]pyrrole-1(2H)-carboxylate (S6-11B) and benzyl 3,3-difluoro-5-(3-((4-methoxybenzyl)oxy)-2,2-dimethyl-3-oxopropyl)-6-oxohexahydropyrrolo[3,4-b]pyrrole-1(2H)-carboxylate (S6-12B)

To a solution of benzyl 3,3-difluoro-5-(3-((4-methoxybenzyl)oxy)-2,2-dimethyl-3-oxopropyl)-hexahydropyrrolo[3,4-b]pyrrole-1(2H)-carboxylate S6-10B (1.30 g, 2.33 mmol, 90% purity) in carbon tetrachloride (15 mL) was added ruthenium(III) chloride (200 mg, 0.964 mmol), sodium periodate (2.50 g, 11.7 mmol) and water (15 mL). The mixture was stirred at 0° C. for 30 minutes. The mixture was diluted with dichloromethane (50 mL) and filtered. The filtrate was poured into water (100 mL) and extracted with dichloromethane (50 mL) three times. The combined organic layers were washed with brine (150 mL), dried over anhydrous sodium sulfate and filtered. The filtrate was concentrated in vacuo and the residue was purified by Prep-Chiral-HPLC (Chiralpak IA 5 um 30*250 mm; Mobile Phase: CO₂:MeOH=75:25 at 50 g/min; Temp: 30° C.; Wavelength: 230 nm) to give benzyl (3aS,6aR)-3,3-difluoro-5-(3-((4-methoxybenzyl)oxy)-2,2-dimethyl-3-oxopropyl)-4-oxohexahydropyrrolo[3,4-b]pyrrole-1(2H)-carboxylate (S6-11B) (210 mg, 90% purity by HNMR, 16% yield, 100% ee) as a yellow oil and benzyl (3aR,6aR)-3,3-difluoro-5-(3-((4-methoxybenzyl)oxy)-2,2-dimethyl-3-oxopropyl)-6-oxohexahydropyrrolo[3,4-b]pyrrole-1(2H)-carboxylate (S6-12B) (280 mg, 90% purity by HNMR, 21% yield, 99.5% ee) as a yellow oil.

S6-11B: ¹H NMR (400 MHz, CDCl₃) δ 7.39-7.27 (m, 6H), 7.24-7.17 (m, 1H), 6.90-6.84 (m, 2H), 5.22-5.13 (m, 2H), 5.05-4.88 (m, 2H), 4.61-4.52 (m, 1H), 4.06-3.90 (m, 1H), 3.80 (s, 3H), 3.73-3.25 (m, 6H), 1.17 (s, 6H).

S6-12B: ¹H NMR (400 MHz, CDCl₃) δ 7.46-7.29 (m, 7H), 6.89-6.87 (m, 2H), 5.25-5.20 (m, 2H), 5.14-5.04 (m, 2H), 5.00-4.79 (m, 1H), 4.19-3.98 (m, 1H), 3.80 (s, 3H), 3.56-3.41 (m, 3H), 3.31-3.28 (m, 1H), 3.24-3.13 (m, 1H), 3.07-2.91 (m, 1H), 1.19-1.16 (m, 6H).

### S6: 3-(3,3-difluoro-4-oxohexahydropyrrolo[3,4-b]pyrrol-5(1H)-yl)-2,2-dimethylpropanoic acid

To a solution of benzyl 3,3-difluoro-5-(3-((4-methoxybenzyl)oxy)-2,2-dimethyl-3-oxopropyl)-4-oxohexahydropyrrolo[3,4-b]pyrrole-1(2H)-carboxylate S6-11B (210 mg, 0.366 mmol, 90% purity) in isopropyl alcohol (5 mL) was added palladium (II) acetate (90 mg, 0.40 mmol) and activated carbon (20 mg). The mixture was heated to 50° C. and stirred for 1 hour under hydrogen (1 atm). After cooled to room temperature the mixture was filtered. The filtrate was concentrated to give the title compound (90 mg, 70% yield, 90% purity by LCMS) as a white solid which was used into next step directly.

LC-MS (ESI): mass calcd. for C₁₁H₁₆F₂N₂O₃ 262.11, m/z found 263.2 [M+H]⁺.

S6-12B was converted to S7 analogously.

### Compound 5A: 3-(1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)-2,2-dimethylpropanoic acid

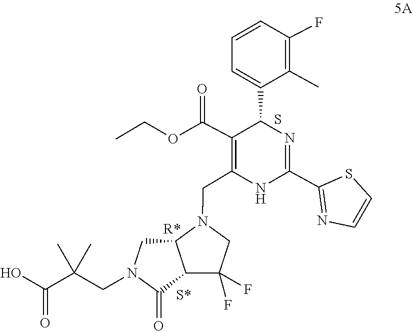

This compound was made according to Typical coupling method 1 from H2-1A with S6. LC-MS (ESI): mass calcd. for C₂₉H₃₂F₃N₅O₅S 619.21, m/z found 620.3 [M+H]⁺. ¹H NMR (400 MHz, CDCl₃) δ 9.19 (s, 1H), 7.81 (d, J=3.2 Hz, 1H), 7.43 (d, J=3.2 Hz, 1H), 7.12-7.02 (m, 2H), 6.93-6.89 (m, 1H), 6.01 (s, 1H), 4.57-4.53 (m, 1H), 4.09-4.00 (m, 3H), 3.87-3.83 (m, 2H), 3.63-3.60 (m, 1H), 3.46-3.39 (m, 1H), 3.33-3.25 (m, 2H), 2.97-2.94 (m, 1H), 2.85-2.76 (m, 1H), 2.53 (s, 3H), 1.36 (s, 3H), 1.33 (s, 3H), 1.11 (t, J=7.2 Hz, 3H).

### Compound 6: 2-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo-[3,2-b]pyrrol-1(2H)-yl)acetic acid

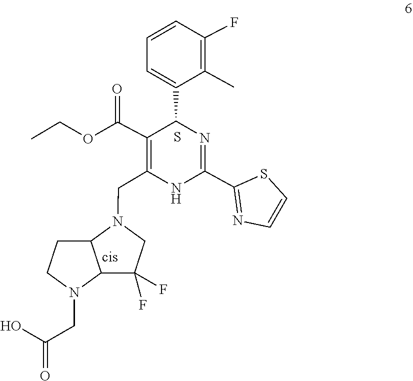

### Preparation of Intermediate S8

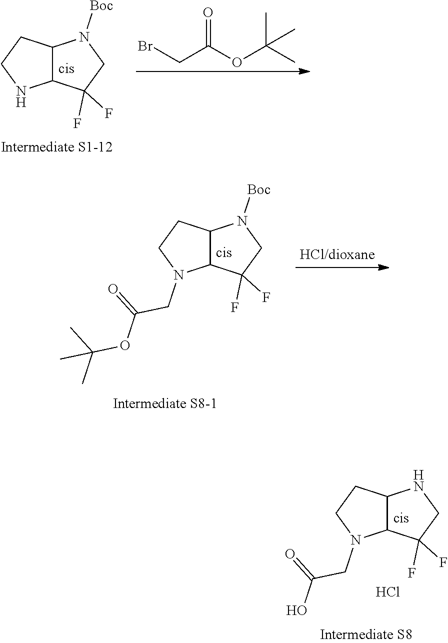

### S8-1: (cis)-tert-butyl 4-(2-(tert-butoxy)-2-oxoethyl)-3,3-difluorohexahydropyrrolo[3,2-b]pyrrole-1(2H)-carboxylate

To a solution of (cis)-tert-butyl 3,3-difluorohexahydropyrrolo[3,2-b] pyrrole-1(2H)-carboxylate S1-12 (230 mg, 90% purity, 0.834 mmol) in N,N-dimethylformamide (4 mL) was added potassium carbonate (345 mg, 2.50 mmol) and tert-butyl bromoacetate (200 mg, 1.025 mmol). After stirred at 35° C. overnight, the mixture was diluted with water (20 mL) and extracted with ethyl acetate (20 mL) twice. The combined organic layers were washed with brine (20 mL), dried over Na₂SO₄₍ₛ₎ and filtered. The filtrate was concentrated to give the title compound (214 mg, 90% purity from ¹H NMR, 64% yield) as colorless oil. ¹H NMR (300 MHz, CDCl₃) δ 4.54-4.43 (m, 1H), 3.91-3.44 (m, 5H), 3.29-3.19 (m, 1H), 2.85-2.74 (m, 1H), 2.37-2.22 (m, 1H), 2.05-1.88 (m, 1H), 1.45 (s, 18H).

### S8: 2-((cis)-6,6-difluorohexahydropyrrolo[3,2-b]pyrrol-1(2H)-yl)acetic acid hydrochloride

A solution of (cis)-tert-butyl 4-(2-(tert-butoxy)-2-oxoethyl)-3,3-difluorohexa-hydropyrrolo[3,2-b]pyrrole-1(2H)-carboxylate S8-1 (154 mg, 90% purity, 0.382 mmol) in 4 M HCl in dioxane (2 mL) was stirred at room temperature for 5 hours. The mixture was concentrated to give the title compound (115 mg, 80% purity, 99% yield) as white solids. ¹H NMR (300 MHz, DMSO-d₆) δ 10.26 (br s, 2H), 4.47 (br s, 1H), 3.75-3.68 (m, 3H), 3.58 (d, J=17.7 Hz, 1H), 3.41 (d, J=17.7 Hz, 1H), 3.29-3.22 (m, 1H), 2.77 (q, J=8.4 Hz, 1H), 2.35-2.11 (m, 2H).

### Compound 6: 2-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo-[3,2-b]pyrrol-1(2H)-yl)acetic acid

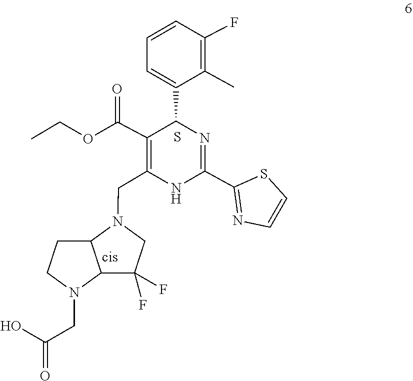

This compound was made according to Typical coupling method 1 from H2-1A with S8. LC-MS (ESI): mass calcd. for C₂₆H₂₈F₃N₅O₄S 563.2, m/z found 564.8. ¹H NMR (400 MHz, CD₃OD) δ 7.90 (d, J=3.2 Hz, 1H), 7.71 (d, J=2.8 Hz, 1H), 7.17-7.08 (m, 2H), 6.95-6.91 (m, 1H), 5.96 (s, 0.5H), 5.95 (s, 0.5H), 4.30-4.24 (m, 1H), 4.17-4.12 (m, 1H), 4.08-4.02 (m, 2H), 3.98-3.89 (m, 1H), 3.75-3.70 (m, 1H), 3.64 (d, J=17.6 Hz, 1H), 3.51 (d, J=17.6 Hz, 1H), 3.48-3.39 (m, 1H), 3.36-3.34 (m, 1H), 3.13-2.98 (m, 1H), 2.91-2.83 (m, 1H), 2.50 (s, 3H), 2.09-1.92 (m, 2H), 1.15-1.11 (m, 3H).

### Compound 6A and 6B: 2-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo-[3,2-b]pyrrol-1(2H)-yl)acetic acid (Single Diastereomer)

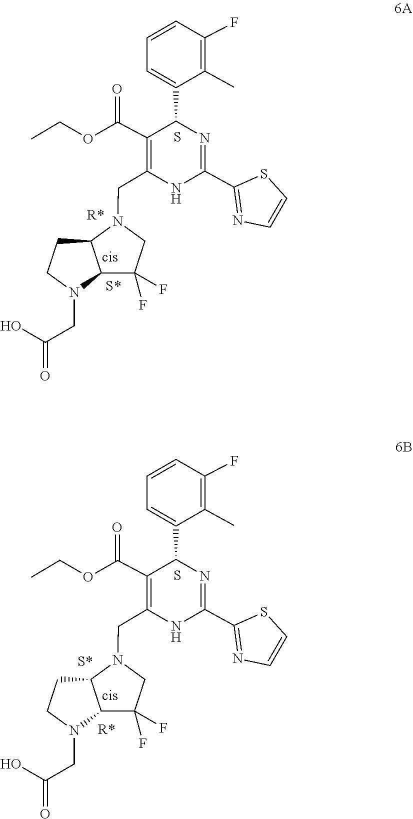

Typical Method 2: Preparation of Allyl Ester and Deprotection

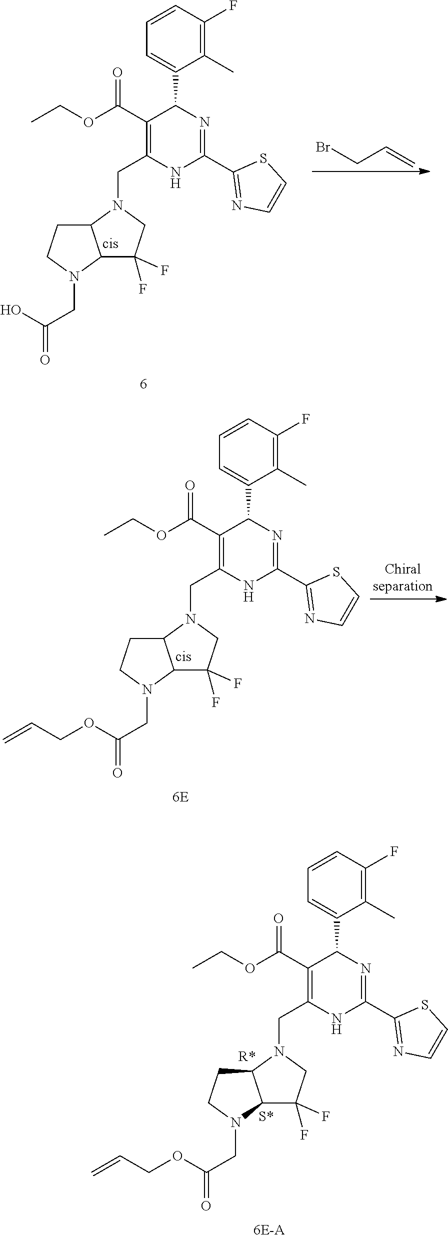

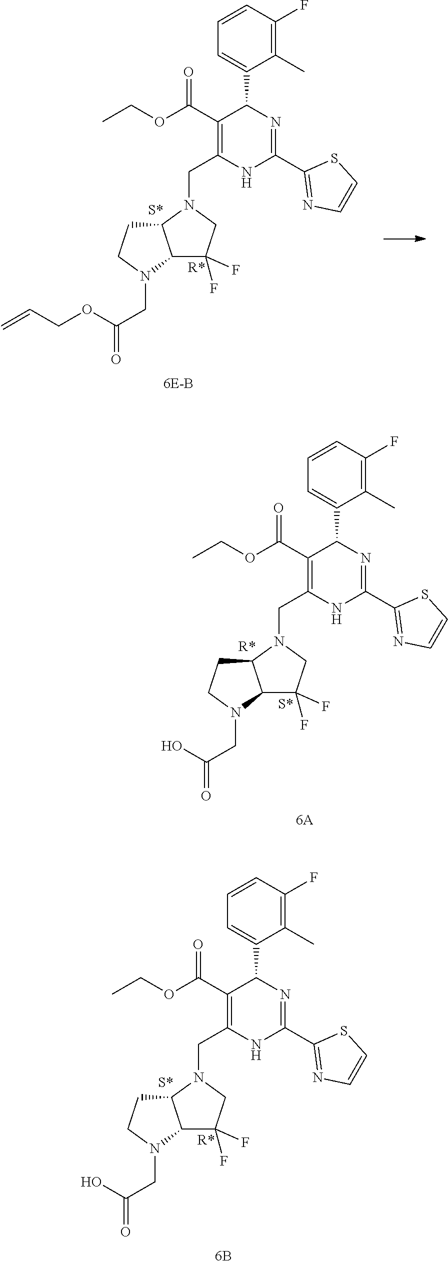 [SMILES: C=CCOC(=O)CN1CC[C@H]2[C@@H]1C(F)(F)CN2CC1=C(C(=O)OCC)[C@H](c2cccc(F)c2C)N=C(c2nccs2)N1 ; CCOC(=O)C1=C(CN2CC(F)(F)[C@@H]3[C@H]2CCN3CC(=O)O)NC(c2nccs2)=N[C@H]1c1cccc(F)c1C ; CCOC(=O)C1=C(CN2CC(F)(F)[C@H]3[C@@H]2CCN3CC(=O)O)NC(c2nccs2)=N[C@H]1c1cccc(F)c1C]

Step 1: Formation of Ally Ester

To a solution of 2-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)acetic acid compound 6 (90 mg, 95% purity, 0.152 mmol) in N,N-dimethylformamide (4 mL) was added potassium carbonate (42 mg, 0.304 mmol) and allyl bromide (22 mg, 0.182 mmol). After stirred at 35° C. overnight, the mixture was concentrated and purified by C18 column (acetonitrile:water=5% to 95%) to give the title compound (64 mg, 95% purity from ¹H NMR, 66% yield) as yellow solids. ¹H NMR (400 MHz, CDCl₃) δ 9.36 (d, J=7.2 Hz, 1H), 7.82-7.80 (m, 1H), 7.39 (d, J=2.8 Hz, 1H), 7.08-7.05 (m, 1H), 7.00-6.98 (m, 1H), 6.90 (t, J=8.8 Hz, 1H), 6.00 (s, 1H), 5.98-5.88 (m, 1H), 5.36-5.25 (m, 2H), 4.63 (d, J=6.0 Hz, 2H), 4.26-4.09 (m, 2H), 4.09-4.02 (m, 2H), 3.94-3.89 (m, 1H), 3.77-3.71 (m, 3H), 3.43-3.23 (m, 2H), 2.99-2.94 (m, 2H), 2.54 (s, 3H), 2.05-1.89 (m, 2H), 1.14-1.10 (m, 3H).

Compound 6E (90 mg, 95% purity, 0.142 mmol) was separated by chiral Prep. HPLC (separation condition: Column: Chiralpak IC 5 um 20*250 mm; Mobile Phase: Hex:IPA:DEA=90:10:0.3 at 15 mL/min; Temp: 30° C.; Wavelength: 254 nm) to give 6E-A (40 mg, 95% purity from ¹H NMR, 44% yield, 99.7% stereopure) and 6E-B (38 mg, 95% purity from ¹H NMR, 42% yield, 99.1% stereopure) as yellow solids. 6E-A: Chiral analysis (Column: Chiralpak IC 5 μm 4.6*250 mm; Mobile Phase: Hex:IPA:DEA=90:10:0.2 at 1.0 mL/min; Temp: 30° C.; Wavelength: 254 nm, R_{T}=8.560 min). ¹H NMR (400 MHz, CDCl₃) δ 9.35 (s, 1H), 7.81 (d, J=2.8 Hz, 1H), 7.40 (d, J=3.2 Hz, 1H), 7.09-7.05 (m, 1H), 6.99 (d, J=3.6 Hz, 1H), 6.92-6.88 (m, 1H), 6.00 (s, 1H), 5.98-5.88 (m, 1H), 5.36-5.25 (m, 2H), 4.63 (d, J=5.6 Hz, 2H), 4.24 (d, J=17.2 Hz, 1H), 4.14 (d, J=17.2 Hz, 1H), 4.08-4.00 (m, 2H), 3.96-3.91 (m, 1H), 3.77-3.73 (m, 1H), 3.71 (s, 2H), 3.39-3.25 (m, 2H), 3.01-2.92 (m, 2H), 2.54 (s, 3H), 2.08-1.94 (m, 2H), 1.11 (t, J=7.2 Hz, 3H).

6E-B: Chiral analysis (Column: Chiralpak IC 5 μm 4.6*250 mm; Mobile Phase: Hex:IPA:DEA=90:10:0.2 at 1.0 mL/min; Temp: 30° C.; Wavelength: 254 nm, R_{T}=9.760 min). ¹H NMR (400 MHz, CDCl₃) δ 9.37 (s, 1H), 7.81 (d, J=2.8 Hz, 1H), 7.39 (d, J=3.2 Hz, 1H), 7.09-7.04 (m, 1H), 6.98 (d, J=3.6 Hz, 1H), 6.92-6.88 (m, 1H), 6.00 (s, 1H), 5.98-5.89 (m, 1H), 5.36-5.25 (m, 2H), 4.63 (d, J=6.0 Hz, 2H), 4.23 (d, J=17.2 Hz, 1H), 4.15 (d, J=17.6 Hz, 1H), 4.09-3.99 (m, 2H), 3.94-3.89 (m, 1H), 3.77-3.74 (m, 1H), 3.71 (s, 2H), 3.43-3.25 (m, 2H), 3.03-2.92 (m, 2H), 2.54 (s, 3H), 2.04-1.88 (m, 2H), 1.12 (t, J=7.2 Hz, 3H).

### Step 2: Deprotection of Allyl Ester

To a solution of compound 6E-A (40 mg, 95% purity, 0.063 mol) in dichloromethane (3 mL) and pyrrolidine (0.2 mL) was added tetrakis(triphenylphosphine)palladium (7 mg, 0.006 mmol) at 0° C. After stirred at 30° C. for 3 hours, the mixture was concentrated and purified by Prep. HPLC (Column: gilson Xbrige C18 (5 um 19*150 mm), Mobile phase A: water (+0.1% trifluoroacetic acid), Mobile phase B: acetonitrile, UV: 214 nm, Flow rate: 15 mL/min, Gradient: 20-60% (% B)) to give a crude product, which was further purified by Prep. HPLC (Column: gilson Xbrige C18 (5 um 19*150 mm), Mobile phase A: water (+0.1% ammonium bicarbonate), Mobile phase B: acetonitrile, UV: 214 nm, Flow rate: 15 mL/min, Gradient: 20-70% (% B)) to give compound 6A (9.8 mg, 96.3% purity, 27% yield) as yellow solids. LC-MS (ESI): mass calcd. for C₂₆H₂₈F₃N₅O₄S 563.2, m/z found 564.1. ¹H NMR (400 MHz, CD₃OD) δ 7.92 (d, J=3.2 Hz, 1H), 7.73 (d, J=2.8 Hz, 1H), 7.19-7.10 (m, 2H), 6.97-6.93 (m, 1H), 5.98 (s, 1H), 4.28 (d, J=16.8 Hz, 1H), 4.17 (d, J=16.4 Hz, 1H), 4.07 (q, J=7.2 Hz, 2H), 4.00-3.95 (m, 1H), 3.77-3.71 (m, 1H), 3.67 (d, J=17.2 Hz, 1H), 3.54 (d, J=17.2 Hz, 1H), 3.50-3.38 (m, 1H), 3.36-3.33 (m, 1H), 3.07-3.00 (m, 1H), 2.90 (q, J=7.6 Hz, 1H), 2.52 (s, 3H), 2.12-1.98 (m, 2H), 1.14 (t, J=7.2 Hz, 3H).

Analogously, compound 6E-B was converted to compound 6B. LC-MS (ESI): mass calcd. for C₂₆H₂₈F₃N₅O₄S 563.2, m/z found 564.2. ¹H NMR (400 MHz, CD₃OD) δ 7.92 (d, J=3.2 Hz, 1H), 7.73 (d, J=3.2 Hz, 1H), 7.19-7.09 (m, 2H), 6.97-6.92 (m, 1H), 5.97 (s, 1H), 4.30 (d, J=16.8 Hz, 1H), 4.16 (d, J=16.4 Hz, 1H), 4.07 (d, J=7.2 Hz, 2H), 3.96-3.91 (m, 1H), 3.78-3.72 (m, 1H), 3.66 (d, J=17.6 Hz, 1H), 3.54 (d, J=17.2 Hz, 1H), 3.50-3.40 (m, 1H), 3.28-3.27 (m, 1H), 3.12-3.04 (m, 1H), 2.90-2.84 (m, 1H), 2.52 (s, 3H), 2.07-1.95 (m, 2H), 1.15 (t, J=6.8 Hz, 3H).

### Compound 7A: 4-(4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo-[3,2-b]pyrrol-1(2H)-yl)-2,2-dimethylbutanoic acid (Single Diastereomer)

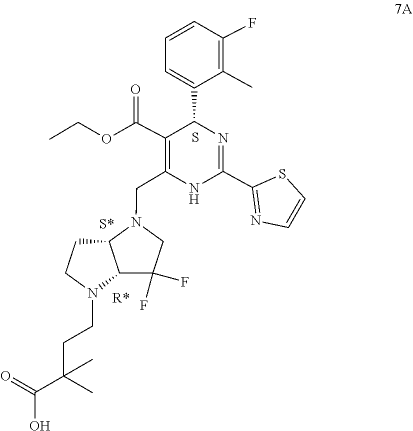

### Preparation of Intermediate S9

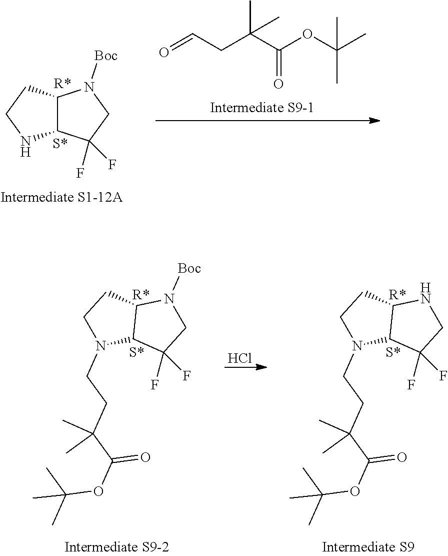

### S9-1: tert-butyl 2,2-dimethyl-4-oxobutanoate

A mixture of tert-butyl 2-bromo-2-methylpropanoate (1 g, 4.482 mmol), N-methyl-N-vinylacetamide (1.3 g, 13.114 mmol), cupric bromide (100 mg, 0.448 mmol), pentamethyldiethylenetriamine (78 mg, 0.45 mmol) and triethylamine (682 mg, 6.740 mmol) in tetrahydrofuran/water (10 mL/1 mL) was stirred at 60° C. overnight under nitrogen. The mixture was added water (20 mL). The mixture was extracted with ethyl acetate (30 mL) three times. The combined organic phases were washed with brine (10 mL), dried over sodium sulfate, filtered and concentrated. The residue was purified by column chromatography on silica gel (petroleum ether:ethyl acetate=10:1) to afford the desired product (400 mg, 48% yield, 95% purity from HNMR) as a colorless oil. ¹H NMR (400 MHz, CDCl₃): 9.75 (s, 1H), 2.57 (s, 2H), 1.44 (s, 9H), 1.25 (s, 6H).

### S9-2: tert-butyl 4-(4-(tert-butoxy)-3,3-dimethyl-4-oxobutyl)-3,3-difluorohexahydropyrrolo[3,2-b]pyrrole-1(2H)-carboxylate

To a solution of tert-butyl 3,3-difluorohexahydropyrrolo[3,2-b]pyrrole-1(2H)-carboxylate S1-12A (100 mg, 0.391 mmol, 97% purity), tert-butyl 2,2-dimethyl-4-oxobutanoate S9-1 (200 mg, 0.966 mmol, 90% purity) in dichloromethane (5 mL) was added 1 M triisopropoxytitanium(IV) chloride in tetrahydrofuran (0.8 mL, 0.8 mmol) dropwise. The mixture was stirred at room temperature for 1 hour. Then sodium triacetoxyborohydride (414 mg, 1.953 mmol) was added and followed by the addition of glacial acetic acid (47 mg, 0.783 mmol). The mixture was stirred at room temperature overnight. LCMS showed the reaction was finished. The mixture was added dichloromethane (30 mL). The organic solution was washed with saturated sodium carbonate solution (10 mL) three times and brine (10 mL), dried over sodium sulfate (s) and filtered. The filtrate was concentrated and the residue was purified by C18 column (acetonitrile:water=40% to 100%) to afford the desired product (155 mg, 54% yield, 58% purity from LCMS) as a yellow oil. LC-MS (ESI): mass calcd. for C₂₁H₃₆F₂N₂O₄ 418.5, m/z found 419.6 [M+H]⁺.

### S9: tert-butyl 4-(6,6-difluorohexahydropyrrolo[3,2-b]pyrrol-1(2H)-yl)-2,2-dimethylbutanoate hydrochloride

To a solution of tert-butyl 4-(4-(tert-butoxy)-3,3-dimethyl-4-oxobutyl)-3,3-difluorohexahydropyrrolo[3,2-b]pyrrole-1(2H)-carboxylate S9-2 (155 mg, 0.259 mmol, 70% purity) in ethyl acetate (3 mL) was added 4 M HCl in ethyl acetate (1 mL, 4 mmol). The mixture was stirred at room temperature for 30 minutes. LCMS showed the reaction was finished. The mixture was concentrated to afford the desired product (90 mg, 97% yield, 100% purity from LCMS) as a white solid. LC-MS (ESI): mass calcd. for C₁₆H₂₈F₂N₂O₂.HCl 354.9, m/z found 319.3 [M+H]⁺.

### Compound 7A-1: ethyl (S)-6-((4-(4-(tert-butoxy)-3,3-dimethyl-4-oxobutyl)-3,3-difluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

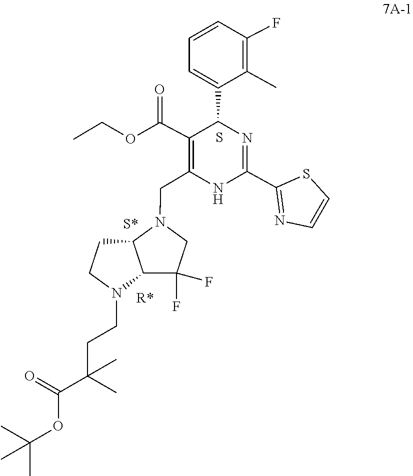

This compound was made from H2-1A with S9 according to Typical coupling method 1. LC-MS (ESI): mass calcd. for C₃₄H₄₄F₃N₅O₄S 675.8, m/z found 676.4 [M+H]⁺.

### Compound 7A: 4-(4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo-[3,2-b]pyrrol-1(2H)-yl)-2,2-dimethylbutanoic acid (Single Diastereomer)

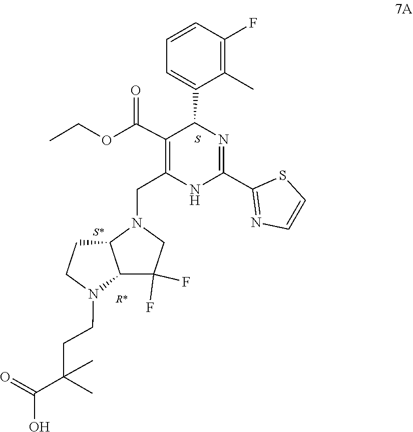

Typical Method 3: Deprotection of Tert-Butyl Ester:

To a solution of Compound 7A-1 (52 mg, 0.077 mmol, 100% purity) in dichloromethane (1 mL) was added trifluoroacetic acid (1 mL). The mixture was stirred at room temperature for 40 minutes. LC-MS showed the reaction was finished. The mixture was concentrated. The residue was purified by C18 column (acetonitrile:water=10% to 70%) to afford the desired product (31.1 mg, 64% yield, 98.5% purity from LC-MS) as a yellow solid. LC-MS (ESI): mass calcd. for C₃₀H₃₆F₃N₅O₄S 619.7, m/z found 620.2 [M+H]⁺. ¹H NMR (400 MHz, CDCl₃): 9.25 (s, 1H), 7.82 (d, J=3.2 Hz, 1H), 7.40 (d, J=3.2 Hz, 1H), 7.10-7.04 (m, 1H), 6.98-6.97 (m, 1H), 6.92-6.88 (m, 1H), 6.00 (s, 1H), 4.28-4.24 (m, 1H), 4.12-3.97 (m, 3H), 3.85-3.80 (m, 1H), 3.45-3.31 (m, 3H), 3.15-3.08 (m, 1H), 3.00-2.92 (m, 1H), 2.71-2.65 (m, 1H), 2.53 (s, 3H), 2.51-2.47 (m, 1H), 2.06-1.91 (m, 3H), 1.71-1.64 (m, 1H), 1.28 (s, 3H), 1.27 (s, 3H), 1.10 (t, J=7.2 Hz, 3H).

### Compound 8A: ethyl (S)-6-(((cis)-3,3-difluoro-4-(2-(methylsulfonamido)-2-oxoethyl)hexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

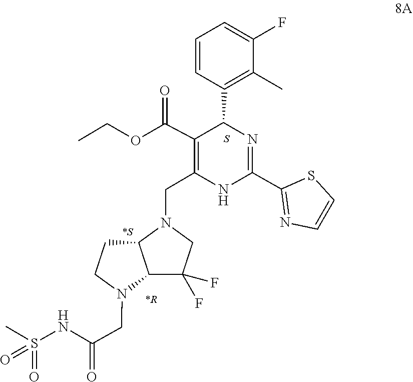

### Preparation of Intermediate S45

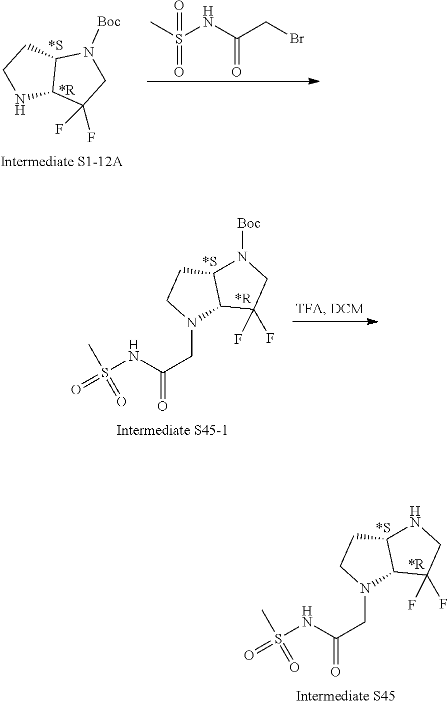

### S45-1: tert-butyl (cis)-3,3-difluoro-4-(2-(methylsulfonamido)-2-oxoethyl)hexahydropyrrolo[3,2-b]pyrrole-1(2H)-carboxylate

To a solution of S1-12A (50 mg, 0.2 mmol) and K₂CO₃ (55.67 mg, 0.4 mmol) in DMF (1 mL, 0.94 g/mL, 12.86 mmol) was added 2-bromo-N-(methylsulfonyl)acetamide (56.57 mg, 0.26 mmol). Then the solution was heated and stirred at 35° C. for 16 hrs. The mixture was filtered and the filtrate was purified by flash column chromatography (Column: C18, 20-35 μm, 100 Å, 40 g) eluting with 5-45% Acetonitrile in water (add 0.05% TFA) to give the title compound (34 mg) as white solid. LC-MS (ESI): mass calcd. For C₁₄H₂₃F₂N₃O₅S 383.1, found m/z 384.1 [M+H]⁺.

S45: 2-((cis)-6,6-difluorohexahydropyrrolo[3,2-b]pyrrol-1(2H)-yl)-N-(methylsulfonyl)acetamide

To a solution of S45-1 (34 mg, 0.089 mmol) in DCM (2 mL, 1.33 g/mL, 31.32 mmol) was added TFA (1 mL, 1.49 g/mL, 13.07 mmol). The mixture was stirred at 20° C. for 1 hr. The mixture was concentrated under reduced pressure to give the title compound (45 mg, crude) which was used in next step directly. LC-MS (ESI): mass calcd. For C₉H₁₅F₂N₃O₃S 283.1, found m/z 284.1 [M+H]⁺.

### Compound 8A: ethyl (S)-6-(((cis)-3,3-difluoro-4-(2-(methylsulfonamido)-2-oxoethyl)hexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

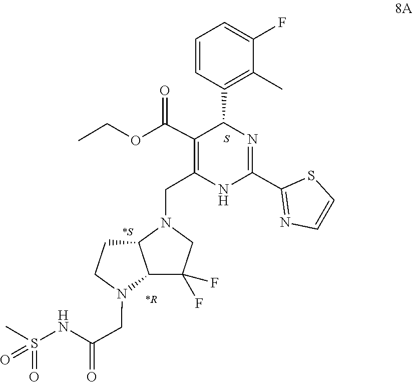

Compound 8A was made from H2-1A and S45 according to typical method 1. LC-MS (ESI): mass calcd. For C₂₇H₃₁F₃N₆O₅S₂ 640.2, m/z 641.2 [M+H]⁺. 1H NMR (400 MHz, CHLOROFORM-d) δ 8.03-8.09 (m, 1H), 7.70-7.77 (m, 1H), 7.11-7.19 (m, 1H), 7.03-7.09 (m, 1H), 6.93-7.03 (m, 1H), 6.14 (s, 1H), 4.35-4.43 (m, 1H), 4.21-4.30 (m, 1H), 4.01-4.12 (m, 3H), 3.53-3.73 (m, 3H), 3.38-3.51 (m, 2H), 3.30-3.35 (m, 3H), 3.19 (q, J=11.17 Hz, 1H), 2.72-2.78 (m, 1H), 2.46 (d, J=1.96 Hz, 3H), 2.16-2.21 (m, 3H), 1.13 (t, J=7.15 Hz, 3H).

### Preparation of T16

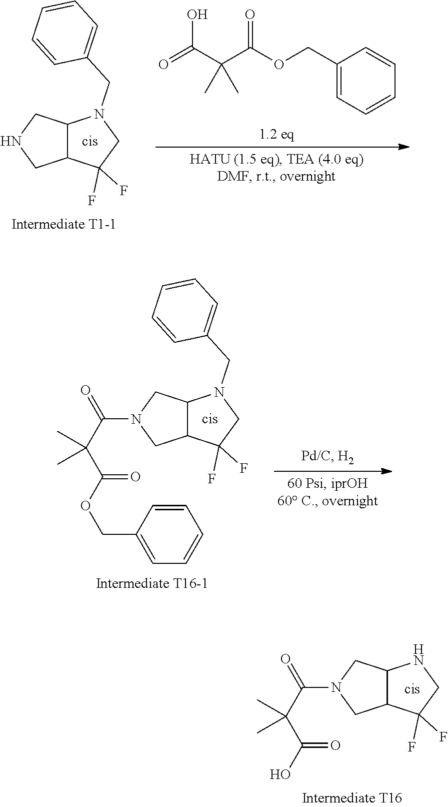 [SMILES: FC1(F)CN(Cc2ccccc2)C2CNCC21 ; CC(C)(C(=O)O)C(=O)OCc1ccccc1 ; CC(C)(C(=O)OCc1ccccc1)C(=O)N1CC2C(C1)C(F)(F)CN2Cc1ccccc1 ; CC(C)(C(=O)O)C(=O)N1CC2NCC(F)(F)C2C1]

### T16-1: (cis)-Benzyl 3-(1-benzyl-3,3-difluorohexahydropyrrolo[3,4-b]pyrrol-5(1H)-yl)-2,2-dimethyl-3-oxopropanoate

To a solution of 3-(benzyloxy)-2,2-dimethyl-3-oxopropanoic acid (311 mg, 90% purity, 1.26 mmol) in N,N-dimethylformamide (4 mL) were added 2-(3H-[1,2,3]triazolo[4,5-b]pyridin-3-yl)-1,1,3,3-tetramethylisouronium hexafluorophosphate (598 mg, 1.57 mmol) and triethylamine (424 mg, 4.19 mmol) at room temperature. The reaction mixture was stirred at room temperature for 1 hour. Then T1-1 (255 mg, 98% purity, 1.05 mmol) in N,N-dimethylformamide (1 mL) was added. The reaction mixture was stirred at room temperature overnight. The reaction mixture was poured into water (20 mL) and extracted with ethyl acetate (60 mL) twice. The combined organic phases were washed with brine (30 mL), dried over Na₂SO₄₍ₛ₎ and filtered. The filtrate was concentrated in vacuum. The residue was purified by C18 column (acetonitrile:water=30% to 95%) to give the title compound (460 mg, 100% purity from LCMS, 99% yield) as colorless oil. LC-MS (ESI): mass calcd. for C₂₅H₂₈F₂N₂O₃ 442.2, m/z found 443.1 [M+H]⁺.

### T16: (cis)-3-(3,3-Difluorohexahydropyrrolo[3,4-b]pyrrol-5(1H)-yl)-2,2-dimethyl-3-oxopropanoic acid

To a solution of T16-1 (240 mg, 95% purity, 0.515 mmol) in isopropanol (10 mL) was added 10% palladium on charcoal wt. (67 mg). The mixture was stirred at 60° C. under hydrogen atmosphere (60 psi) overnight. The mixture was filtered and the filtrate was concentrated under reduced pressure to give the title compound (210 mg, 90% purity from LCMS, 77% yield) as yellow oil. LC-MS (ESI): mass calcd. for C₁₁H₁₆F₂N₂O₃ 262.1, m/z found 263.1 [M+H]⁺.

### Compound 9: (cis)-3-(1-(((S)-5-(Ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)-2,2-dimethyl-3-oxopropanoic acid

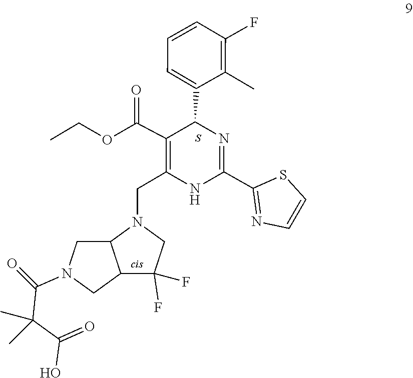

This compound was made according to Typical coupling method 1 from H2-1A with T16. Purified by C18 column (acetonitrile:water=40% to 60%) to give the desired compound (33.2 mg, 96% purity from LCMS, 14% yield) as yellow solids. LC-MS (ESI): mass calcd. for C₂₉H₃₂F₃N₅O₅S 619.2, m/z found 620.3 [M+H]⁺. ¹H NMR (400 MHz, CD₃OD) δ 7.78 (s, 1H), 7.58 (t, J=3.6 Hz, 1H), 7.10-6.96 (m, 2H), 6.82 (t, J=8.8 Hz, 1H), 5.86 (s, 0.5H), 5.83 (s, 0.5H), 4.31-4.10 (m, 1.5H), 4.05-3.92 (m, 3.5H), 3.83-3.64 (m, 2H), 3.57-3.52 (m, 1H), 3.51-3.30 (m, 2H), 3.08-2.88 (m, 2H), 2.40 (s, 3H), 1.30-1.12 (m, 5H), 1.05-1.01 (m, 3H), 0.91 (s, 1H).

### Compound 9A: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)-2,2-dimethyl-3-oxopropanoic acid (Single Diastereomer)

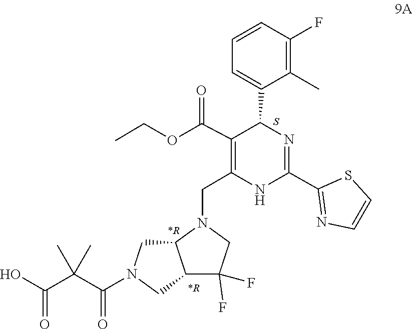

Separated Compound 9 using chiral SFC (Column: Chiralpak IE 5 μm 30*250 mm; Mobile Phase: MeOH 30% at 3 mL/min; Temp: 40° C.; Wavelength: 254 nm). ¹H NMR (400 MHz, CDCl₃) δ ppm 8.95-9.16 (m, 1H), 7.65-7.81 (m, 1H), 7.30-7.38 (m, 1H), 7.02-7.12 (m, 1H), 6.93-7.01 (m, 1H), 6.79-6.92 (m, 1H), 5.90-6.04 (m, 1H), 4.25-4.52 (m, 1H), 3.83-4.23 (m, 5H), 3.51-3.81 (m, 3H), 3.27-3.46 (m, 1H), 3.00-3.25 (m, 1H), 2.73-2.96 (m, 1H), 2.44-2.57 (m, 3H), 1.30-1.43 (m, 6H), 1.07 (br t, J=7.03 Hz, 3H).

### Preparation of T20

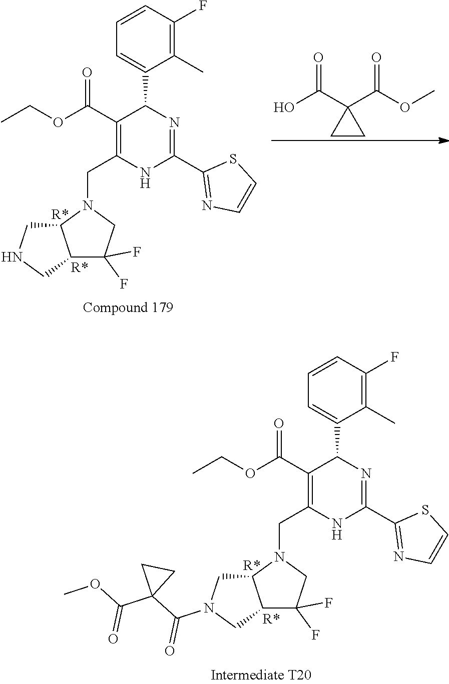

### (S)-Ethyl 6-(((cis)-3,3-difluoro-5-(1-(methoxycarbonyl)cyclopropanecarbonyl)-hexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate

To a solution of 1-(methoxycarbonyl)cyclopropanecarboxylic acid (35 mg, 0.194 mmol) in N,N-dimethylformamide (5 mL) was added Compound 179 (70 mg, 90% purity, 0.109 mmol), 2-(7-aza-1H-benzotriazole-1-yl)-1,1,3,3-tetramethyluronium hexafluorophosphate (85 mg, 0.224 mmol) and N,N-diisopropylethylamine (90 mg, 0.696 mmol) at room temperature. After stirred at room temperature overnight, the mixture was diluted with dichloromethane (40 mL), washed with water (40 mL), brine (40 mL), dried over Na₂SO₄₍ₛ₎, filtered, concentrated and purified by C18 column (acetonitrile:water=5% to 95%) twice to give the title compound (70 mg, 91.5% purity, 52% yield) as yellow solids. LC-MS (ESI): mass calcd. for C₃₀H₃₂F₃N₅O₅S 631.2, m/z found 632.4 [M+H]⁺.

### Compound 10: 1-((cis)-1-(((S)-5-(Ethoxycarbonyl)-6-(3-fluoro-2-methylphenyl)-2-(thiazol-2-yl)-3,6-dihydropyrimidin-4-yl)methyl)-3,3-difluorooctahydropyrrolo[3,4-b]pyrrole-5-carbonyl)cyclopropanecarboxylic acid (Single Diastereomer)

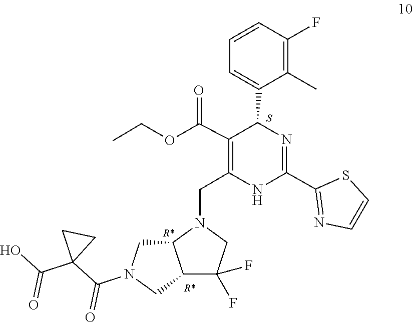

This compound was made from T20 according to typical method 4. LC-MS (ESI): mass calcd. for C₂₉H₃₀F₃N₅O₅S 617.2, m/z found 618.3 [M+H]⁺. ¹H NMR (400 MHz, CDCl₃) δ 8.53-8.42 (m, 1H), 8.02-7.84 (m, 1H), 7.46 (d, J=3.2 Hz, 1H), 7.14-7.13 (m, 1H), 7.00-6.98 (m, 1H), 6.94-6.90 (m, 1H), 6.00 (s, 1H), 4.73-4.47 (m, 1H), 4.36-4.21 (m, 1H), 4.06-4.01 (m, 3H), 3.92-3.74 (m, 2H), 3.56-3.55 (m, 1H), 3.43-3.35 (m, 2H), 3.22-3.12 (m, 1H), 2.84-2.74 (m, 1H), 2.51 (s, 3H), 1.58-1.18 (m, 4H), 1.09 (t, J=7.2 Hz, 3H).

### Compound 11: 3-(4-(((S)-5-(ethoxycarbonyl)-6-(3-fluoro-2-methylphenyl)-2-(thiazol-2-yl)-3,6-dihydropyrimidin-4-yl)methyl)-6-fluorohexahydropyrrolo[3,2-b]pyrrol-1(2H)-yl)-2,2-dimethylpropanoic acid

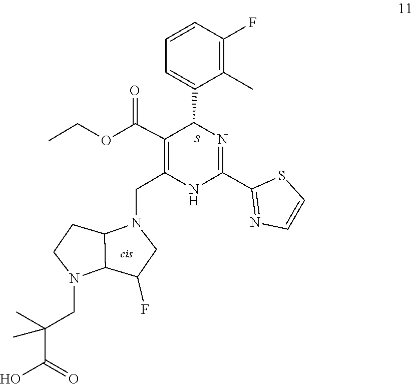

### Preparation of Intermediate S30

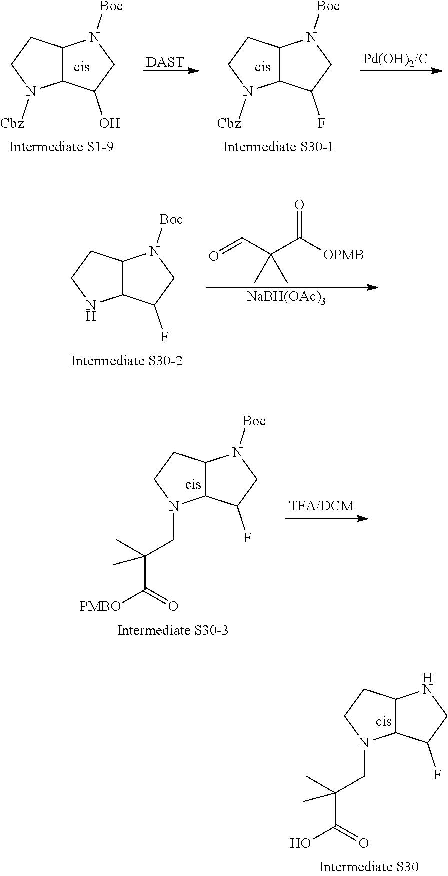

### S30-1: 4-Benzyl 1-tert-butyl 3-fluorotetrahydropyrrolo[3,2-b]pyrrole-1,4(2H,5H)-dicarboxylate

To a solution of (cis,trans)-4-benzyl 1-tert-butyl 3-hydroxytetrahydropyrrolo[3,2-b]pyrrole-1,4(2H,5H)-dicarboxylate S1-9 (2.00 g, 90% purity, 4.97 mmol) in dichloromethane (40 mL) was added diethylaminosulfur trifluoride (2.40 g, 14.9 mmol) at −78° C. under nitrogen atmosphere. After stirred at −78° C. for 2 hours, the mixture was warmed to 20° C. and stirred for another 16 hours. Then it was quenched with saturated sodium bicarbonate aqueous solution (100 mL). The organic phase was separated and the aqueous layer was extracted with dichloromethane (30 mL) for three times. The combined organic phases were washed with brine (30 mL), dried over Na₂SO₄₍ₛ₎ and filtered. The filtrate was concentrated to give a residue, which was purified by silica gel column chromatography (petroleum ether:ethyl acetate=20:1 to 5:1) to give the title compound (1.20 g, 90% purity from ¹H NMR, 59.7% yield) as colorless oil. LC-MS (ESI): mass calcd. for C₁₉H₂₅FN₂O₄ 364.2, m/z found 309.4 [M+H−56]⁺. ¹H NMR (400 MHz, CDCl₃) δ 7.44-7.29 (m, 5H), 5.29-5.09 (m, 2.7H), 5.02-4.93 (m, 0.3H), 4.58-4.40 (m, 2H), 4.01-3.72 (m, 2H), 3.42-3.34 (m, 0.5H), 3.27-3.23 (m, 0.5H), 3.15-3.06 (m, 1H), 2.39-2.31 (m, 0.5H), 2.22-2.17 (m, 0.5H), 1.98-1.85 (m, 1H), 1.48 (s, 4H), 1.47 (s, 5H).

### S30-2: tert-Butyl 3-fluorohexahydropyrrolo[3,2-b]pyrrole-1(2H)-carboxylate

To a solution of 4-benzyl 1-tert-butyl 3-fluorotetrahydropyrrolo[3,2-b]pyrrole-1,4(2H,5H)-dicarboxylate S30-1 (1.20 g, 90% purity, 2.96 mmol) in isopropanol (25 mL) was added 20% palladium hydroxide on activated carbon (600 mg, 0.854 mmol) under nitrogen atmosphere. After stirred at 40° C. under hydrogen atmosphere (H₂ balloon) for 2 hours, the reaction mixture was filtered and the filtrate was concentrated to give title compound (700 mg, 90% purity from ¹H NMR, 92.3% yield) as colorless oil. ¹H NMR (400 MHz, CDCl₃) δ 4.91-4.90 (m, 0.5H), 4.78-4.77 (m, 0.5H), 4.52-4.49 (m, 0.5H), 4.42-4.40 (m, 0.5H), 3.96-3.76 (m, 2H), 3.50-3.47 (m, 0.5H), 3.40-3.37 (m, 0.5H), 3.05-2.92 (m, 1H), 2.81-2.74 (m, 1H), 2.09-1.88 (m, 3H), 1.48 (s, 5H), 1.47 (s, 4H).

### S30-3: tert-Butyl 3-fluoro-4-(3-((4-methoxybenzyl)oxy)-2,2-dimethyl-3-oxopropyl)hexahydropyrrolo[3,2-b]pyrrole-1(2H)-carboxylate

To a mixture of tert-butyl 3-fluorohexahydropyrrolo[3,2-b]pyrrole-1(2H)-carboxylate S30-2 (700 mg, 90% purity, 2.74 mmol) in dichloromethane (10 mL) was added acetic acid (0.7 mL), 4-methoxybenzyl 2,2-dimethyl-3-oxopropanoate (1.00 g, 95% purity, 4.02 mmol) and 1 M triisopropoxytitanium(IV) chloride in dichloromethane (5.6 mL, 5.6 mmol). The mixture was stirred at 20° C. for 20 minutes, then sodium triacetoxy-borohydride (2.89 g, 13.6 mmol) was added. After stirred at 20° C. for 16 hours, the reaction mixture was quenched with saturated sodium bicarbonate aqueous solution (30 mL) and filtered. The filtrate was extracted with dichloromethane (20 mL) for three times. The combined organic phases were washed with brine (20 mL), dried over Na₂SO₄₍ₛ₎ and filtered. The filtrate was concentrated to give a residue, which was purified by C18 (acetonitrile:water=20% to 70%) to give the title compound (1.30 g, 90% purity from ¹H NMR, 95% yield) as colorless oil. LC-MS (ESI): mass calcd. for C₂₄H₃₅FN₂O₅ 450.3, m/z found 451.3 [M+H]⁺. ¹H NMR (400 MHz, CDCl₃) δ 7.32-7.22 (m, 2H), 6.88 (d, J=8.4 Hz, 2H), 5.07-4.99 (m, 2H), 4.80-4.78 (m, 0.5H), 4.68-4.65 (m, 0.5H), 4.38-4.31 (m, 0.5H), 4.28-4.21 (m, 0.5H), 3.87-3.84 (m, 0.2H), 3.81 (s, 3H), 3.79-3.77 (m, 0.2H), 3.75-3.64 (m, 0.6H), 3.50-3.26 (m, 1H), 3.14-3.09 (m, 1H), 3.01-2.92 (m, 1H), 2.88-2.82 (m, 1H), 2.64-2.57 (m, 1H), 2.24-2.06 (m, 2H), 1.76-1.67 (m, 1H), 1.45 (s, 9H), 1.19 (s, 3H), 1.16 (s, 3H).

Racemic S30-3 (400 mg, 90% purity, 0.799 mmol) was separated by chiral HPLC (Column: Chiralpak IF 5 μm 20*250 mm; Mobile Phase: Hex:EtOH=80:20 at 15 mL/min; Temp: 35° C.; Wavelength: 230 nm) to give the title compounds S30-3A (160 mg, 90% purity from ¹HNMR, 40% yield, 100% stereopure) and S30-3B (170 mg, 90% purity from ¹HNMR, 43% yield, 100% stereopure) as colorless oil.

S30-3A: LC-MS (ESI): mass calcd. for C₂₄H₃₅FN₂O₅ 450.3, m/z found 451.3 [M+H]⁺. Chiral analysis (Column: Chiralpak IF 5 μm 4.6*250 mm; Mobile Phase: Hex:EtOH=80:20 at 1 mL/min; Temp: 30° C.; Wavelength: 230 nm; R_{T}=6.377 min). ¹H NMR (400 MHz, CDCl₃) δ 7.28-7.26 (m, 2H), 6.88 (d, J=8.8 Hz, 2H), 5.07-5.00 (m, 2H), 4.81-4.67 (m, 1H), 4.37-4.24 (m, 1H), 3.87-3.85 (m, 0.2H), 3.81 (s, 3H), 3.79-3.65 (m, 0.8H), 3.49-3.27 (m, 1H), 3.15-3.10 (m, 1H), 2.99-2.94 (m, 1H), 2.88-2.83 (m, 1H), 2.65-2.60 (m, 1H), 2.24-2.08 (m, 2H), 1.79-1.65 (m, 1H), 1.46 (s, 9H), 1.19 (s, 3H), 1.16 (s, 3H).

S30-3B: LC-MS (ESI): mass calcd. for C₂₄H₃₅FN₂O₅ 450.3, m/z found 451.3 [M+H]⁺. Chiral analysis (Column: Chiralpak IF 5 μm 4.6*250 mm; Mobile Phase: Hex:EtOH=80:20 at 1 mL/min; Temp: 30° C.; Wavelength: 230 nm; R_{T}=10.175 min). ¹H NMR (400 MHz, CDCl₃) δ 7.29-7.26 (m, 2H), 6.88 (d, J=8.4 Hz, 2H), 5.07-5.00 (m, 2H), 4.81-4.68 (m, 1H), 4.38-4.24 (m, 1H), 3.88-3.85 (m, 0.2H), 3.81 (s, 3H), 3.79-3.65 (m, 0.8H), 3.50-3.34 (m, 1H), 3.16-3.11 (m, 1H), 3.00-2.95 (m, 1H), 2.89-2.83 (m, 1H), 2.65-2.61 (m, 3H), 2.25-2.10 (m, 2H), 1.84-1.61 (m, 1H), 1.46 (s, 9H), 1.20 (s, 3H), 1.16 (s, 3H).

### S30: 4-(2-Carboxy-2-methylpropyl)-3-fluorooctahydropyrrolo[3,2-b]pyrrol-1-ium trifluoroacetate

To a solution of tert-butyl 3-fluoro-4-(3-((4-methoxybenzyl)oxy)-2,2-dimethyl-3-oxopropyl)hexahydropyrrolo[3,2-b]pyrrole-1(2H)-carboxylate S30-3 (100 mg, 90% purity, 0.2 mmol) in dichloromethane (2 mL) was added trifluoroacetic acid (2 mL). After stirred at 20° C. for 1 hour, the reaction mixture was concentrated to give the title compound (200 mg, 24% purity, 73% yield) as yellow solids. LC-MS (ESI): mass calcd. for C₁₃H₁₉F₄N₂O₃ 327.1, m/z found 231.1 [M−TFA+H]⁺.

S30B was prepared from S30-3B analogously.

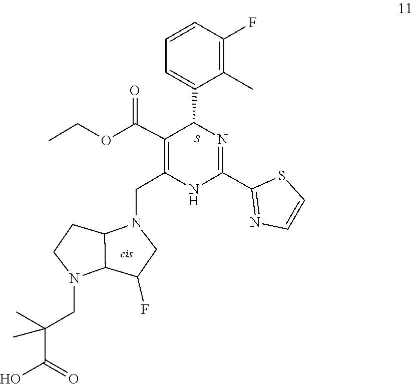

This compound was made from H2-1A with S30 according to Typical coupling method 1. Purified by prep-HPLC (Column: Waters Xbridge C18 (5 μm 19*150 mm), Mobile Phase A: water (0.1% ammonium acetate), Mobile Phase B: acetonitrile, UV: 214 nm, Flow rate: 15 mL/min, Gradient: 20-80% (% B)) and further purified by by C18 (acetonitrile:water (0.1% ammonium bicarbonate)=10% to 50%) to give the title compound (24.9 mg, 98.6% purity, 28% yield) as yellow solids. LC-MS (ESI): mass calcd. for C₂₉H₃₅F₂N₅O₄S 587.2, m/z found 588.3 [M+H]⁺. ¹H NMR (400 MHz, DMSO-d₆) δ 12.18 (br s, 1H), 9.53 (d, J=6.8 Hz, 1H), 7.99-7.95 (m, 1H), 7.92-7.91 (m, 1H), 7.21-7.15 (m, 1H), 7.07-7.01 (m, 2H), 5.87 (d, J=1.6 Hz, 0.9H), 5.76 (s, 0.1H), 4.96-4.91 (m, 0.5H), 4.83-4.78 (m, 0.5H), 4.22-4.06 (m, 2H), 3.97 (q, J=6.8 Hz, 2H), 3.79-3.68 (m, 1H), 3.28-3.05 (m, 2H), 3.01-2.91 (m, 2H), 2.83-2.79 (m, 1H), 2.72-2.68 (m, 1H), 2.45 (s, 3H), 2.36-2.27 (m, 1H), 1.87-1.55 (m, 2H), 1.14-1.01 (m, 9H).

### Compound 11B: (cis)-3-(4-(((S)-5-(ethoxycarbonyl)-6-(3-fluoro-2-methylphenyl)-2-(thiazol-2-yl)-3,6-dihydropyrimidin-4-yl)methyl)-6-fluorohexahydropyrrolo[3,2-b]pyrrol-1(2H)-yl)-2,2-dimethylpropanoic acid

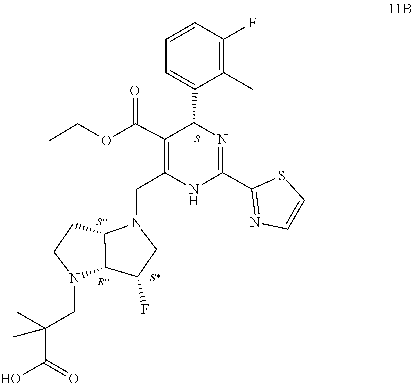

Compound 11B was made from H2-1A with S30B according to Typical coupling method 1. Purified by prep-HPLC (Column: Waters Xbridge C18 (5 μm 19*150 mm), Mobile Phase A: water (0.1% ammonium acetate), Mobile Phase B: acetonitrile, UV: 214 nm, Flow rate: 15 mL/min, Gradient: 25-80% (% B)) and further purified by C18 (acetonitrile:water (0.1% ammonium bicarbonate)=10% to 50%) to give the title compound (60.8 mg, 98% purity, 71.5% yield) as yellow solids. LC-MS (ESI): mass calcd. for C₂₉H₃₅F₂N₅O₄S 587.2, m/z found 588.3 [M+H]⁺. ¹H NMR (400 MHz, DMSO-d₆) δ 12.14 (br s, 1H), 9.54 (s, 1H), 7.97 (d, J=2.8 Hz, 1H), 7.91 (d, J=3.2 Hz, 1H), 7.21-7.16 (m, 1H), 7.06-7.01 (m, 2H), 5.87 (s, 0.9H), 5.76 (d, J=2.8 Hz, 0.1H), 4.93-4.80 (m, 1H), 4.14 (s, 2H), 3.97 (q, J=6.8 Hz, 2H), 3.79-3.74 (m, 1H), 3.29-3.24 (m, 1H), 3.18-3.06 (m, 1H), 2.99-2.91 (m, 2H), 2.81 (d, J=13.2 Hz, 1H), 2.71 (d, J=13.2 Hz, 1H), 2.45 (s, 2.8H), 2.40 (s, 0.2H), 2.37-2.31 (m, 1H), 1.89-1.78 (m, 1H), 1.71-1.62 (m, 1H), 1.12 (s, 3H), 1.11 (s, 3H), 1.05 (t, J=7.2 Hz, 3H).

### Preparation of Intermediate T1 and T2

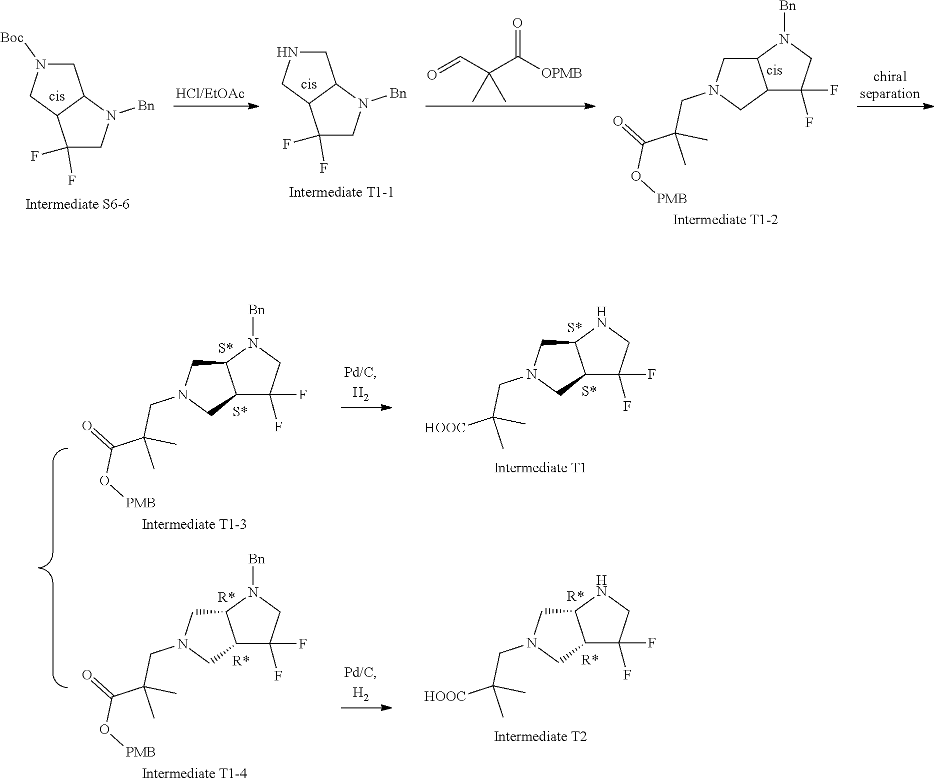

### T1-1: (cis)-1-benzyl-3,3-difluorooctahydropyrrolo[3,4-b]pyrrole

To a solution of cis-tert-butyl 1-benzyl-3,3-difluorohexahydropyrrolo[3,4-b]pyrrole-5(1H)-carboxylate S6-6 (500 mg, 1.43 mmol, 97% purity) in ethyl acetate (4 mL) was added HCl (20 mL, 5.0M, in ethyl acetate) at room temperature. The reaction mixture was stirred at room temperature for 1 hour. The reaction mixture was concentrated in vacuum. The residue was poured into water (2 mL), basified with 1 M Sodium hydroxide aqueous solution to pH=11. The mixture was freeze-dried to give a yellow solid. The yellow solid was poured into dichlormethane (30 mL) and filtered. The filtrate was concentrated in vacuum to give the desired compound (349 mg, 99% yield, 98% purity from LCMS) as a yellow solid. LC-MS (ESI): mass calcd. for C₁₃H₁₆F₂N₂ 238.3, m/z found 239.1.

### T1-2: (cis)-4-methoxybenzyl 3-(1-benzyl-3,3-difluorohexahydropyrrolo[3,4-b]pyrrol-5(1H)-yl)-2,2-dimethylpropanoate

This compound was made from T1-1 and 4-methoxybenzyl 2,2-dimethyl-3-oxopropanoate according to typical method 5. ¹HNMR (400 MHz, DMSO-d₆): 7.30-7.27 (m, 6H), 7.24-7.20 (m, 1H), 6.87-6.85 (m, 2H), 5.05 (s, 2H), 3.78 (s, 3H), 3.73-3.60 (m, 1H), 3.57-3.56 (m, 2H), 3.14-3.01 (m, 2H), 2.97-2.89 (m, 2H), 2.82-2.73 (m, 1H), 2.78-2.69 (m, 2H), 2.32-2.29 (m, 1H), 2.23-2.13 (m, 1H), 1.20 (s, 6H).

### Intermediates T1-3 and T1-4: 4-Methoxybenzyl 3-((cis)-1-benzyl-3,3-difluorohexahydropyrrolo[3,4-b]pyrrol-5(1H)-yl)-2,2-dimethylpropanoate

A racemic mixture of 4-methoxybenzyl 3-((cis)-1-benzyl-3,3-difluorohexahydropyrrolo[3,4-b]pyrrol-5(1H)-yl)-2,2-dimethylpropanoate T1-2 (2.60 g, 90% purity, 5.10 mmol) was separated by chiral preparative HPLC (Column: Chiralpak IG 5 μm 30*250 mm; Mobile Phase: Hex:EtOH=95:5 at 30 mL/min; Temp: 30° C.; Wavelength: 254 nm) to give T1-3 (1.10 g, 95% purity by ¹H NMR, 45% yield, 99.9% stereopure) and T1-4 (1.10 g, 95% purity by ¹H NMR, 45% yield, 99.8% stereopure) as yellow oil.

T1-3: Chiral analysis (Column: Chiralpak IG 5 μm 4.6*250 mm; Mobile Phase: Hex:EtOH=95:5 at 1.0 mL/min; Temp: 30° C.; Wavelength: 214 nm, R_{T}=6.841 min). ¹H NMR (400 MHz, CDCl₃) δ 7.32-7.22 (m, 7H), 6.86 (d, J=8.0 Hz, 2H), 5.08-5.01 (m, 2H), 3.78 (s, 3H), 3.73-3.70 (m, 1H), 3.59-3.56 (m, 2H), 3.10-2.71 (m, 5H), 2.60-2.52 (m, 2H), 2.29-2.24 (m, 1H), 2.12-2.08 (m, 1H), 1.20 (s, 3H), 1.19 (s, 3H).

T1-4: Chiral analysis (Column: Chiralpak IG 5 μm 4.6*250 mm; Mobile Phase: Hex:EtOH 30=95:5 at 1.0 mL/min; Temp: 30° C.; Wavelength: 214 nm, R_{T}=7.907 min). ¹H NMR (400 MHz, CDCl₃) δ 7.32-7.22 (m, 7H), 6.86 (d, J=8.0 Hz, 2H), 5.08-5.01 (m, 2H), 3.77 (s, 3H), 3.73-3.69 (m, 1H), 3.59-3.56 (m, 2H), 3.10-2.71 (m, 5H), 2.60-2.52 (m, 2H), 2.28-2.24 (m, 1H), 2.12-2.08 (m, 1H), 1.20 (s, 3H), 1.19 (s, 3H).

### Intermediates T1: 3-((cis)-3,3-Difluorohexahydropyrrolo[3,4-b]pyrrol-5(1H)-yl)-2,2-dimethylpropanoic acid

To a solution of T1-3 (1.10 g, 95% purity, 2.28 mmol) in isopropyl alcohol (20 mL) was added 10% wt. palladium on charcoal (262 mg) at room temperature. The mixture was heated to 60° C. and stirred for 16 hours under hydrogen atmosphere (60 psi). The reaction mixture was filtered, and the filtrate was concentrated in vacuum to give the desired product (607 mg, 90% purity from ¹H NMR, 97% yield) as white solid. ¹H NMR (400 MHz, DMSO-d₆) δ 3.83-3.76 (m, 1H), 3.04-2.91 (m, 3H), 2.74-2.58 (m, 2H), 2.46 (s, 2H), 2.40-2.36 (m, 1H), 2.29-2.25 (m, 1H), 1.07 (s, 6H).

### Intermediates T2: 3-((cis)-3,3-Difluorohexahydropyrrolo[3,4-b]pyrrol-5(1H)-yl)-2,2-dimethylpropanoic acid

To a solution of T1-4 (1.10 g, 95% purity, 2.28 mmol) in isopropyl alcohol (20 mL) was added 10% wt. palladium on charcoal (262 mg) at room temperature. The mixture was heated to 60° C. and stirred for 16 hours under hydrogen atmosphere (60 psi). Then it was filtered, and the filtrate was concentrated in vacuum to give the desired product (600 mg, 90% purity from ¹H NMR, 95% yield) as white solids. ¹H NMR (400 MHz, DMSO-d₆) δ 3.85-3.76 (m, 1H), 3.04-2.93 (m, 3H), 2.74-2.60 (m, 2H), 2.47 (s, 2H), 2.40-2.36 (m, 1H), 2.30-2.22 (m, 1H), 1.04 (s, 6H).

### Preparation of Intermediate T3

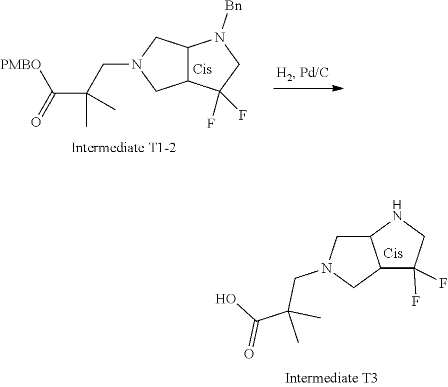

This compound was made from T1-2 analogous to T2. ¹HNMR (400 MHz, DMSO-d6): 3.80-3.70 (m, 1H), 3.05-3.02 (m, 1H), 2.99-2.92 (m, 2H), 2.73-2.66 (m, 1H), 2.50-2.46 (m, 3H), 2.40-2.22 (m, 2H), 1.04 (s, 6H).

### Compound 12: (cis)-3-(1-(((S)-5-(ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)-2,2-dimethylpropanoic acid

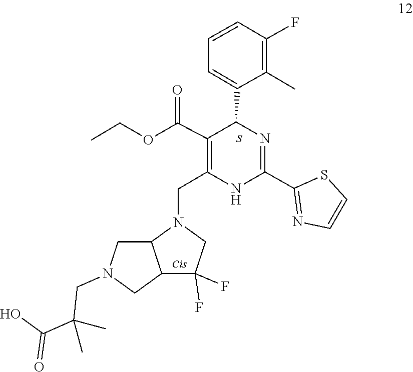

This compound was made according to Typical coupling method 1 from H12-1A with T3. Purified by Prep. HPLC (Column: Waters Xbrige C18 (5 μm 19*150 mm), Mobile phase A: water (0.1% ammonium bicarbonate), Mobile phase B: acetonitrile, UV: 214 nm, Flow rate: 15 mL/min, Gradient: 20-80% (% B)) to give desired compound (25 mg, 23% yield, 96% purity from LCMS) as yellow solid. LC-MS (ESI): mass calcd. for C₂₉H₃₄F₃N₅O₄S 605.7, m/z found 606.2. HNMR (400 MHz, DMSO-d₆): 9.12 (br s, 1H), 7.89-7.87 (m, 1H), 7.40-7.39 (m, 1H), 7.09-7.07 (m, 1H), 7.05-6.99 (m, 1H), 6.91-6.86 (m, 1H), 5.99 (d, J=7.2 Hz, 1H), 4.44-4.25 (m, 1H), 4.12-4.01 (m, 3H), 3.75-3.72 (m, 1H), 3.55-3.31 (m, 3H), 3.23-2.95 (m, 2H), 2.84-2.74 (m, 2H), 2.71-2.64 (m, 2H), 2.55 (s, 3H), 1.26-1.22 (m, 6H), 1.14-1.08 (m, 3H).

### Compound 12B: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)-2,2-dimethylpropanoic acid (Single Diastereomer)

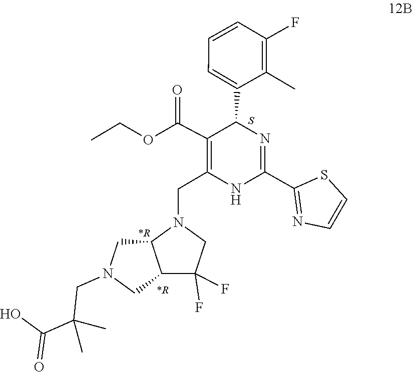

This compound was made using the procedure similar to Compound 12 by replacing T3 with T2. Purified by Prep. HPLC (Column: Waters Xbrige C18 (5 μm 19*150 mm), Mobile phase A: water (0.1% ammonium bicarbonate), Mobile phase B: acetonitrile, UV: 214 nm, Flow rate: 15 mL/min, Gradient: 20-80% (% B)) to give desired compound (389 mg, 99.5% purity, 59% yield) as a yellow solid. LC-MS (ESI): mass calcd. for C₂₉H₃₄F₃N₅O₄S 605.2, m/z found 606.3 [M+H]⁺. ¹H NMR (400 MHz, CDCl₃) δ 9.14 (br s, 1H), 7.87 (d, J=3.2 Hz, 1H), 7.41 (d, J=3.2 Hz, 1H), 7.14-7.06 (m, 1H), 7.01-6.97 (m, 1H), 6.93-6.86 (m, 1H), 6.01 (s, 1H), 4.40-4.28 (m, 1H), 4.10-3.97 (m, 3H), 3.78-3.70 (m, 1H), 3.53-3.51 (m, 1H), 3.37-3.35 (m, 2H), 3.15-2.86 (m, 2H), 2.77-2.72 (m, 2H), 2.68-2.62 (m, 2H), 2.55-2.52 (m, 3H), 1.27 (s, 3H), 1.23 (s, 3H), 1.10 (t, J=7.2 Hz, 3H).

### Compound 13B: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-6-oxohexahydropyrrolo[3,4-b]pyrrol-5(1H)-yl)-2,2-dimethylpropanoic acid (Single Diastereomer)

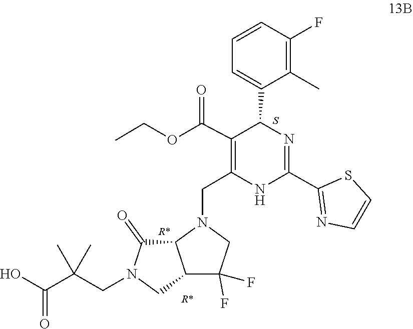

This compound was made using the procedure similar to Compound 42 by replacing H5-1A with H2-1A. Purified by Prep. HPLC (Column: Xbridge C18 (5 μm 19*150 mm), Mobile Phase A: water (0.1% trifluoroacetic acid), Mobile Phase B: acetonitrile, UV: 214 nm, Flow rate: 15 mL/min, Gradient: 20-75% (% B)) to give the title compound (53 mg, 96.3% purity) as light yellow solids. LC-MS (ESI): mass calcd. for C₂₉H₃₂F₃N₅O₅S 619.7, m/z found 620.2 [M+H]⁺. ¹H NMR (400 MHz, CDCl₃) δ 8.99 (s, 1H), 7.92 (d, J=3.2 Hz, 1H), 7.40 (d, J=3.2 Hz, 1H), 7.12-7.03 (m, 2H), 6.90 (t, J=8.4 Hz, 1H), 5.99 (s, 1H), 4.72 (d, J=16.0 Hz, 1H), 4.33 (d, J=16.0 Hz, 1H), 4.08-3.98 (m, 3H), 3.92 (dd, J=10.8, 3.2 Hz, 1H), 3.76 (d, J=9.2 Hz, 1H), 3.49 (t, J=9.6 Hz, 1H), 3.30 (q, J=10.4 Hz, 1H), 3.25-3.14 (m, 1H), 3.11-3.02 (m, 1H), 2.96 (d, J=14.0 Hz, 1H), 2.52 (s, 3H), 1.37 (s, 3H), 1.31 (s, 3H), 1.11 (t, J=7.2 Hz, 3H).

### Preparation of Intermediate T17

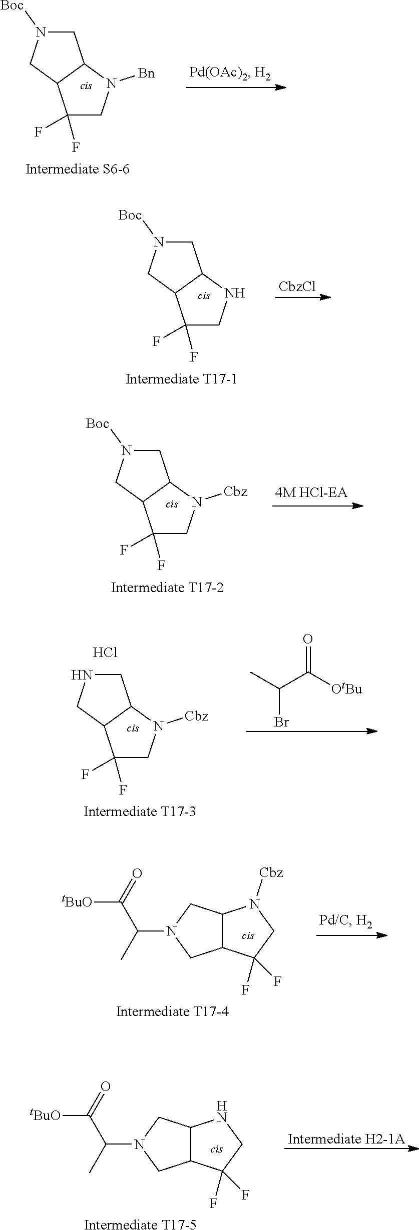

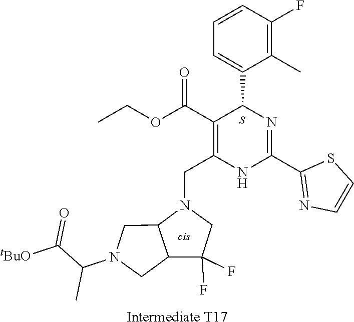

### T17-1: (cis)-tert-Butyl 3,3-difluorohexahydropyrrolo[3,4-b]pyrrole-5(1H)-carboxylate

To a solution of S6-6 (1.60 g, 90% purity, 4.26 mmol) in isopropyl alcohol (10 mL) was added palladium (II) acetate (290 mg, 1.29 mmol) and active carbon (2.61 g, 163 mmol). The mixture was heated to 60° C. and stirred overnight under hydrogen atmosphere (60 psi). After cooled to room temperature, the mixture was filtered and the filtrate was concentrated to give the desired compound (1.15 g, 90% purity from ¹H NMR, 98% yield) as yellow oil. ¹HNMR (400 MHz, CDCl₃) δ 4.10-4.00 (m, 3H), 3.73-3.69 (m, 1H), 3.50-3.44 (m, 2H), 3.30-3.18 (m, 2H), 2.97-2.84 (m, 1H), 1.46 (s, 9H).

### T17-2: (cis)-1-Benzyl 5-tert-butyl 3,3-difluorohexahydropyrrolo[3,4-b]pyrrole-1,5-dicarboxylate

To a solution of T17-1 (1.15 g, 90% purity, 4.17 mmol) in tetrahydrofuran (5 mL) was added saturated sodium bicarbonate aqueous solution (5 mL) and benzyl carbonochloridate (1.07 g, 6.25 mmol). The mixture was stirred at room temperature overnight. The mixture was poured into water (10 mL) and extracted with ethyl acetate (50 mL) for three times. The combined organic phases were washed with brine (30 mL), dried over anhydrous Na₂SO₄₍ₛ₎ and filtered. The filtrate was concentrated to give the desired compound (1.36 g, 90% purity from ¹H NMR, 77% yield) as yellow oil. LC-MS (ESI): mass calcd. for C₁₉H₂₄F₂N₂O₄ 382.2, m/z found 327.1 [M+H−56]⁺. ¹HNMR (400 MHz, CDCl₃) δ 7.36-7.26 (m, 5H), 5.14 (s, 2H), 4.54-4.49 (m, 1H), 4.01-3.91 (m, 1H), 3.76-3.53 (m, 5H), 3.12 (s, 1H), 1.45 (s, 9H).

### T17-3: (cis)-Benzyl 3,3-difluorohexahydropyrrolo[3,4-b]pyrrole-1(2H)-carboxylate hydrochloride

To a solution of T17-2 (1.36 g, 90% purity, 3.20 mmol) in ethyl acetate (5 mL) was added 4 M hydrochloride in ethyl acetate (5 mL). The mixture was stirred at room temperature overnight. The mixture was concentrated to give the desired compound (1.10 g, 82% purity from LCMS, 97% yield) as white solids. LC-MS (ESI): mass calcd. for C₁₄H₁₇ClF₂N₂O₂ 318.1, m/z found 283.0 [M+H−HCl]+.

### T17-4: (cis)-Benzyl 5-(1-(tert-butoxy)-1-oxopropan-2-yl)-3,3-difluorohexahydropyrrolo[3,4-b]pyrrole-1(2H)-carboxylate

A solution of T17-3 (100 mg, 95% purity, 0.30 mmol), N,N-diisopropylethylamine (300 mg, 2.3 mmol) and tert-butyl 2-bromopropanoate (150 mg, 0.72 mmol) in N,N-dimethylformamide (5 mL) was stirred at room temperature overnight. The mixture was purified by C18 column (acetonitrile:water=5% to 50%) to give the title compound (100 mg, 95% purity, 77% yield) as yellow oil. LC-MS (ESI): mass calcd. for C₂₁H₂₈F₂N₂O₄ 410.2, m/z found 411.2 [M+H]⁺.

### T17-5: tert-Butyl 2-((cis)-3,3-difluorohexahydropyrrolo[3,4-b]pyrrol-5(1H)-yl)propanoate

To a solution of T17-4 (100 mg, 95% purity, 0.231 mmol) in isopropanol (10 mL) was added 10% wt palladium on charcoal (24 mg, 0.023 mmol). The mixture was heated and stirred at 35° C. overnight under hydrogen balloon. The mixture was filtered and the filtrate was concentrated to give a crude (60 mg, 90% purity from ¹H NMR, 84% yield) as yellow oil. ¹H NMR (400 MHz, CDCl₃) δ 3.98-3.90 (m, 1H), 3.28-2.98 (m, 4H), 2.81-2.56 (m, 4H), 1.82 (br s, 1H), 1.46 (s, 9H), 1.30-1.27 (s, 3H).

### T17: (4S)-Ethyl 6-(((cis)-5-(1-(tert-butoxy)-1-oxopropan-2-yl)-3,3-difluorohexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate

This compound was made from T17-5 and H2-1A according to typical method 1. LC-MS (ESI): mass calcd. for C₃₁H₃₈F₃N₅O₄S 633.3, m/z found 634.4 [M+H]⁺.

### Compound 14: 2-((cis)-1-(((S)-5-(Ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)propanoic acid

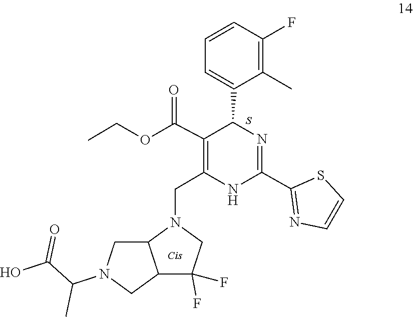

This compound was made from T17 according to typical method 3. LC-MS (ESI): R_{T}=3.226 & 3.308 min, mass calcd. for C₂H₃₀F₃N₅O₄S 577.2, m/z found 578.2 [M+H]⁺. ¹H NMR (400 MHz, CD₃OD) δ 7.98-7.88 (m, 1H), 7.87-7.67 (m, 1H), 7.25-7.02 (m, 2H), 6.97-6.87 (m, 1H), 5.95 (s, 1H), 4.37-3.95 (m, 4H), 3.87-3.44 (m, 5H), 3.24-2.94 (m, 4H), 2.47 (s, 3H), 1.63-1.36 (m, 3H), 1.11-1.07 (m, 3H).

### Compound 15: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)-2-methylpropanoic acid

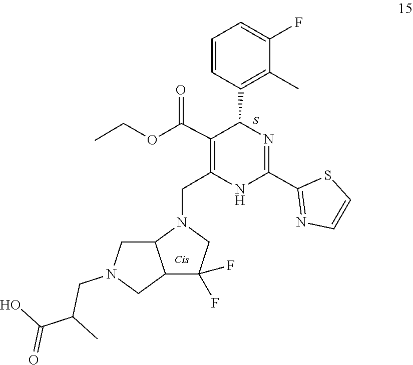

This compound was made using the procedure similar to Compound 12 by replacing 4-methoxybenzyl 2,2-dimethyl-3-oxopropanoate with tert-butyl 2-methyl-3-oxopropanoate and hydrolyze the tert-butyl ester with TFA in DCM. LC-MS (ESI): mass calcd. for C₂₈H₃₂F₃N₅O₄S 591.2, m/z found 592.2 [M+H]⁺. ¹H NMR (400 MHz, DMSO-d₆) δ 12.34 (br s, 1H), 9.58 (s, 0.1H), 9.44-9.41 (m, 0.9H), 8.00-7.90 (m, 2H), 7.20-7.15 (m, 1H), 7.06-7.01 (m, 2H), 5.88-5.87 (m, 0.9H), 5.77 (s, 0.1H), 4.28-4.08 (m, 2H), 3.98 (q, J=7.2 Hz, 2H), 3.90-3.74 (m, 1H), 3.14-2.99 (m, 5H), 2.69-2.55 (m, 1H), 2.45 (s, 3H), 2.40-2.33 (m, 2H), 2.28-2.16 (m, 2H), 1.09-1.05 (m, 6H).

### Preparation of Intermediate T5

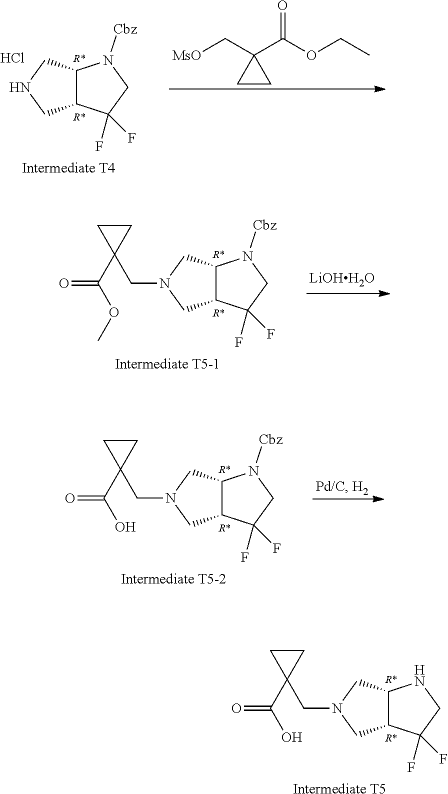

### T5-1: (cis)-Benzyl 5-((1-(ethoxycarbonyl)cyclopropyl)methyl)-3,3-difluorohexahydropyrrolo[3,4-b]pyrrole-1(2H)-carboxylate

To a solution of ethyl 1-(((methylsulfonyl)oxy)methyl)cyclopropanecarboxylate (120 mg, 90% purity, 0.486 mmol) in acetonitrile (5 mL) were added potassium carbonate (117 mg, 0.847 mmol) and T4 (100 mg, 9000 purity, 0.282 mmol) at room temperature. After stirred at 80° C. overnight under nitrogen atmosphere, the mixture was cooled to room temperature and concentrated to give a residue, which was quenched with water (20 mL) slowly and extracted with ethyl acetate (20 mL) for three times. The combined organic layers were washed with brine (20 mL), dried over Na₂SO₄₍ₛ₎, filtered and concentrated under reduced pressure to give a residue, which was purified by C18 column (acetonitrile:water=40% to 95%) to give the title compound (100 mg, 90% purity from ¹H NMR, 78% yield) as yellow oil. ¹H NMR (400 MHz, CDCl₃) δ 7.39-7.32 (m, 5H), 5.21-5.10 (m, 2H), 4.55-4.46 (m, 1H), 4.12-3.95 (m, 3H), 3.61-3.48 (m, 1H), 3.19-3.09 (m, 1.6H), 2.99-2.86 (m, 1.4H), 2.68-2.58 (m, 2H), 2.39-2.23 (m, 2H), 1.22-1.18 (m, 5H), 0.78-0.71 (m, 2H).

### T5-2: 1-(((cis)-1-((benzyloxy)carbonyl)-3,3-difluorohexahydropyrrolo[3,4-b]pyrrol-5(1H)-yl)methyl)cyclopropanecarboxylic acid

To a solution of T5-1 (100 mg, 90% purity, 0.220 mmol) in tetrahydrofuran (3 mL) was added a solution of lithium hydroxide monohydrate (28 mg, 0.67 mmol) in water (1.5 mL). After stirred at room temperature overnight under nitrogen atmosphere, the reaction mixture was acidified with 1 M hydrochloride aqueous solution (about 2 mL) to pH=4-5 and extracted with ethyl acetate (20 mL) for three times. The combined organic layers were dried over anhydrous Na₂SO₄ and filtered. The filtrate was concentrated under reduced pressure to afford the residue, which was purified by C18 column (acetonitrile:water=5% to 95%) to give the title compound (57 mg, 90% purity from ¹H NMR, 61% yield) as white solids. ¹H NMR (400 MHz, CDCl₃) δ 14.00 (br s, 1H), 7.40-7.33 (m, 5H), 5.25-5.09 (m, 2H), 4.69-4.57 (m, 1H), 4.19-4.00 (m, 1H), 3.73-3.62 (m, 1H), 3.54-3.27 (m, 1.6H), 3.21-2.99 (m, 1.4H), 2.74-2.38 (m, 4H), 1.43-1.34 (m, 2H), 0.69-0.62 (m, 2H).

### T5: 1-(((cis)-3,3-difluorohexahydropyrrolo[3,4-b]pyrrol-5(1H)-yl)methyl)cyclopropanecarboxylic acid

To a solution of T5-2 (57 mg, 90% purity, 0.14 mmol) in methanol (5 mL) was added 10% palladium on charcoal wt. (30 mg, 0.028 mmol) at room temperature. After stirred at room temperature under hydrogen atmosphere (balloon) overnight, the mixture was filtered through a pad of celite. The filtrate was concentrated under reduced pressure to give the title compound (40 mg, 80% purity from ¹H NMR, 96% yield) as white solids. ¹H NMR (400 MHz, DMSO-d₆) δ 3.93-3.87 (m, 0.5H), 3.58-3.39 (m, 1.5H), 3.23-3.15 (m, 1H), 3.04-2.95 (m, 2H), 2.83-2.67 (m, 2H), 2.61-2.54 (m, 1H), 2.45-2.33 (m, 2H), 1.04-0.98 (m, 2H), 0.67-0.63 (m, 2H).

### Compound 16B: 1-(((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)methyl)cyclopropane-1-carboxylic acid (Single Diastereomer)

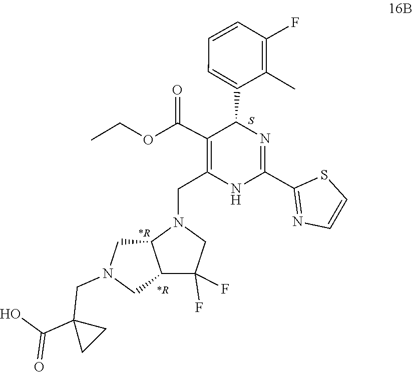

This compound was made according to Typical coupling method 1 from H2-1A with T5. Purified by C18 column (acetonitrile:water (0.1% ammonium bicarbonate)=5% to 95%) to give the title compound (24 mg, 96.7% purity, 35% yield) as yellow solids. LC-MS (ESI): mass calcd. for C₂₉H₃₂F₃N₅O₄S 603.2, m/z found 604.2 [M+H]⁺. ¹H NMR (400 MHz, DMSO-d₆) δ 9.61 (d, J=3.2 Hz, 0.2H), 9.43 (s, 0.8H), 8.00-7.90 (m, 2H), 7.20-7.15 (m, 1.2H), 7.06-7.01 (m, 1.8H), 5.88 (s, 0.8H), 5.77 (d, J=3.2 Hz, 0.2H), 4.22-4.06 (m, 2H), 3.97 (q, J=7.2 Hz, 2H), 3.88-3.81 (m, 1H), 3.17-3.05 (m, 4H), 2.72-2.58 (m, 2.3H), 2.44-2.27 (m, 5.7H), 1.07-1.02 (m, 5H), 0.75-0.72 (m, 1.6H), 0.66-0.61 (m, 0.4H).

### Compound 17B: 4-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)-2,2-dimethylbutanoic acid (Single Diastereomer)

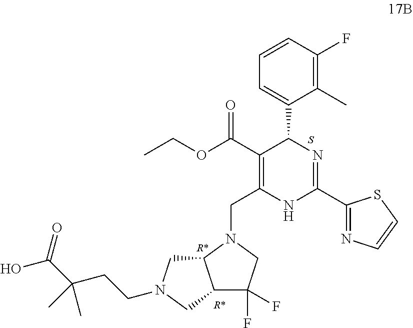

This compound was made using the procedure similar to Compound 12 by replacing 4-methoxybenzyl 2,2-dimethyl-3-oxopropanoate with tert-butyl 2,2-dimethyl-4-oxobutanoate. Purified by C18 column (acetonitrile:water=5% to 75%) to give the desired product (60 mg, 96.8% purity) as yellow solids. LC-MS (ESI): mass calcd. for C₃₀H₃₆F₃N₅O₄S 619.7, m/z found 620.2 [M+H]⁺. ¹H NMR (400 MHz, CD₃OD) δ 7.94 (d, J=3.2 Hz, 1H), 7.75 (d, J=3.2 Hz, 1H), 7.20-7.13 (m, 2H), 6.98-6.94 (m, 1H), 5.99 (s, 1H), 4.19-4.16 (m, 2H), 4.09 (q, J=7.2 Hz, 2H), 3.90-3.88 (m, 1H), 3.65-3.48 (m, 3H), 3.27-3.23 (m, 1H), 3.15-3.08 (m, 1H), 2.97-2.73 (m, 4H), 2.51 (s, 3H), 1.83-1.79 (m, 2H), 1.16-1.13 (m, 9H).

### Preparation of Intermediate T6A and T6B

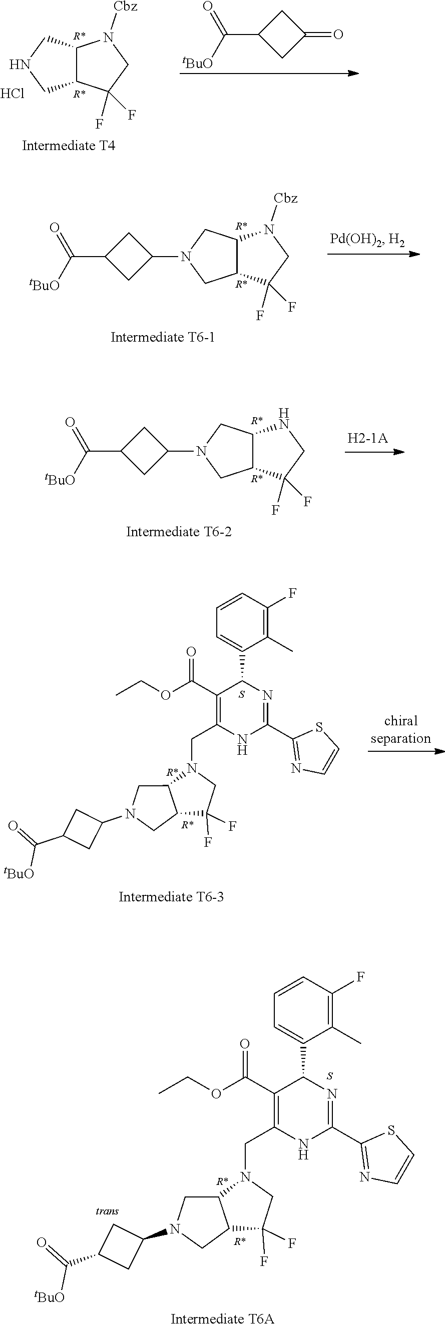

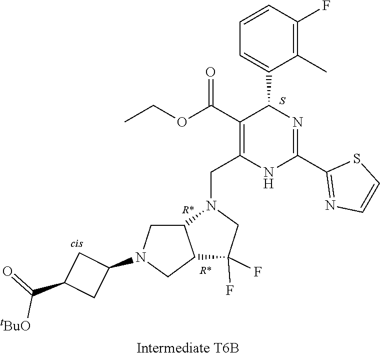

### T6-1: (cis)-Benzyl 5-(3-(tert-butoxycarbonyl)cyclobutyl)-3,3-difluorohexahydropyrrolo[3,4-b]pyrrole-1(2H)-carboxylate

To a solution of T4 (100 mg, 90% purity, 0.282 mmol) in methanol (2 mL) was added triethylamine (29 mg, 0.287 mmol), zinc chloride (4 mg, 0.029 mmol) and tert-butyl 3-oxocyclobutanecarboxylate (61 mg, 0.358 mmol). After stirred at 65° C. for 5 hours, the mixture was then cooled to 0° C., and sodium cyanoborohydride (37 mg, 0.589 mmol) was added portion-wise. After stirred at 25° C. overnight, the reaction was concentrated under reduced pressure and purified by C18 column (acetonitrile:water (0.1% ammonium bicarbonate)=65% to 75%) to afford the title compound (110 mg, 90% purity from ¹H NMR, 80% yield) as colorless oil. LC-MS (ESI): mass calcd. for C₂₃H₃₀F₂N₂O₄ 436.5, m/z found 437.3 [M+H]⁺. ¹H NMR (400 MHz, CDCl₃) δ 7.39-7.30 (m, 5H), 5.20-5.10 (m, 2H), 4.57-4.47 (m, 1H), 4.10-3.95 (m, 1H), 3.73-3.60 (m, 1H), 3.04-2.63 (m, 5H), 2.30-2.06 (m, 5.6H), 2.04-2.01 (m, 0.4H), 1.45-1.43 (m, 9H).

### T6-2: tert-Butyl 3-((cis)-3,3-difluorohexahydropyrrolo[3,4-b]pyrrol-5(1H)-yl)cyclobutanecarboxylate

To the solution of T6-1 (110 mg, 90% purity, 0.227 mmol) in methanol (5 mL) was added 20% palladium hydroxide on activated carbon (0.5 g). After stirred at 60° C. under hydrogen atmosphere (H₂ balloon) overnight, the mixture was filtered and the filtrate was concentrated to give the title compound (60 mg, 54% purity, 47% yield) as colorless oil. LC-MS (ESI): mass calcd. for C₁₅H₂₄F₂N₂O₂ 302.4, m/z found 303.4 [M+H]⁺.

### T6-3: (S)-Ethyl 6-(((cis)-5-(3-(tert-butoxycarbonyl)cyclobutyl)-3,3-difluorohexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate

To the solution of H2-1A (50 mg, 95% purity, 0.108 mmol) in dichloromethane (2 mL) was added triethanolamine (100 mg, 0.67 mmol). A solution of T6-2 (60 mg, 54% purity, 0.107 mmol) in dichloromethane (1 mL) was added at 40° C. After stirred at 40° C. overnight, the mixture was concentrated and purified by C18 column (acetonitrile:water=70% to 95%) to give the title compound (50 mg, 95% purity from ¹H NMR, 66% yield) as yellow solids. LC-MS (ESI): mass calcd. for C₃₃H₄₀F₃N₅O₄S 659.8, m/z found 660.5 [M+H]⁺. ¹H NMR (400 MHz, CDCl₃) δ 9.54 (s, 0.4H), 9.49 (s, 0.6H), 7.84 (d, J=2.8 Hz, 1H), 7.41-7.39 (m, 1H), 7.10-7.05 (m, 1H), 7.00-6.98 (m, 1H), 6.92-6.88 (m, 1H), 6.00 (s, 1H), 4.27-4.16 (m, 2H), 4.10-3.99 (m, 2H), 3.81-3.73 (m, 1H), 3.45-3.35 (m, 1H), 3.14-2.94 (m, 5H), 2.71-2.63 (m, 1H), 2.54 (d, J=2 Hz, 3H), 2.31-2.15 (m, 6H), 1.43 (s, 3.6H), 1.41 (s, 5.4H), 1.12 (t, J=7.2 Hz, 3H).

### T6A and T6B: (S)-Ethyl 6-(((cis)-5-((trans)-3-(tert-butoxycarbonyl)cyclobutyl)-3,3-difluorohexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate

T6-3 (50 mg, 95% purity, 0.072 mmol) was separated by chiral prep. HPLC (Column: Chiralpak IB 5 μm 20*250 mm; Mobile Phase: Hex:IPA=90:10 at 18 mL/min; Temp: 30° C.; Wavelength: 254 nm) to give the title compounds T6A (20 mg, 90% purity from ¹H NMR, 38% yield) and T6B (25 mg, 90% purity from ¹H NMR, 47% yield) as colorless oil.

T6A: LC-MS (ESI): mass calcd. for C₃₃H₄₀F₃N₅O₄S 659.8, m/z found 660.8 [M+H]⁺. Chiral analysis (Column: Chiralpak IB 5 μm 4.6*250 mm; Mobile Phase: Hex:IPA=90:10 at 1.0 mL/min; Temp: 30° C.; Wavelength: 254 nm, R_{T}=5.519 min). ¹H NMR (400 MHz, CDCl₃) δ 9.56 (s, 1H), 7.84 (d, J=3.2 Hz, 1H), 7.40 (d, J=2.8 Hz, 1H), 7.08-7.04 (m, 1H), 7.00-6.98 (m, 1H), 6.92-6.88 (m, 1H), 6.01 (s, 1H), 4.26 (d, J=18 Hz, 1H), 4.17 (d, J=16 Hz, 1H), 4.08-3.99 (m, 2H), 3.79-3.72 (m, 1H), 3.45-3.34 (m, 1H), 3.16-2.84 (m, 6H), 2.54 (d, J=1.6 Hz, 3H), 2.27-2.14 (m, 6H), 1.43 (s, 9H), 1.12 (t, J=7.2 Hz, 3H).

T6B: LC-MS (ESI): mass calcd. for C₃₃H₄₀F₃N₅O₄S 659.8, m/z found 660.6 [M+H]⁺. Chiral analysis (Column: Chiralpak IB 5 μm 4.6*250 mm; Mobile Phase: Hex:IPA=90:10 at 1.0 mL/min; Temp: 30° C.; Wavelength: 254 nm, R_{T}=6.682 min). ¹H NMR (400 MHz, CDCl₃) δ 9.49 (s, 1H), 7.84 (d, J=3.2 Hz, 1H), 7.40 (d, J=3.2 Hz, 1H), 7.10-7.05 (m, 1H), 7.00-6.98 (m, 1H), 6.92-6.88 (m, 1H), 6.00 (s, 1H), 4.23 (d, J=17.6 Hz, 1H), 4.17 (d, J=16.8 Hz, 1H), 4.09-3.99 (m, 2H), 3.79-3.76 (m, 1H), 3.45-3.35 (m, 1H), 3.07-2.95 (m, 5H), 2.70-2.66 (m, 1H), 2.54 (d, J=1.6 Hz, 3H), 2.33-2.17 (m, 6H), 1.41 (s, 9H), 1.12 (t, J=7.2 Hz, 3H).

### Compound 18A: (trans)-3-((cis)-1-(((S)-5-(Ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)cyclobutanecarboxylic acid (Single Diastereomer)

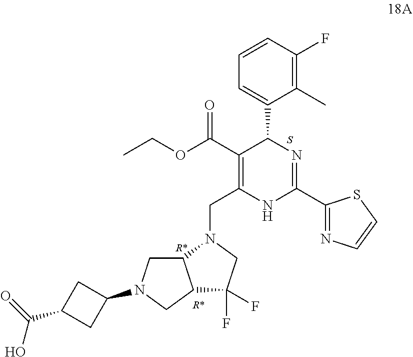

To the solution of T6A (20 mg, 90% purity, 0.065 mmol) in dichloromethane (1 mL) was added trifluoroacetic acid (1 mL) at 0° C. After stirred at 25° C. for 1 hour, the mixture was concentrated and purified by C18 column (acetonitrile:water (add 0.02% ammonium bicarbonate)=35% to 45%) to give the title compound (12 mg, 99.5% purity, 72% yield) as yellow solids. LC-MS (ESI): mass calcd. for C₂₉H₃₂F₃N₅O₄S 603.6, m/z found 604.3 [M+H]⁺. ¹H NMR (400 MHz, CDCl₃+ D₂O) δ 7.84 (d, J=3.2 Hz, 1H), 7.43 (d, J=2.0 Hz, 1H), 7.10-7.06 (m, 1H), 7.02-7.06 (m, 1H), 6.93-6.89 (m, 1H), 6.00 (s, 1H), 4.26 (d, J=18 Hz, 1H), 4.11-3.99 (m, 3H), 3.85-3.76 (m, 1H), 3.51-3.33 (m, 2H), 3.27-3.25 (m, 1H), 3.14-3.08 (m, 3H), 3.03-2.97 (m, 1H), 2.74-2.59 (m, 2H), 2.52 (s, 3H), 2.48-2.33 (m, 4H), 1.11 (t, J=7.2 Hz, 3H).

### Compound 18B: (cis)-3-((cis)-1-(((S)-5-(Ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)cyclobutanecarboxylic acid (Single Diastereomer)

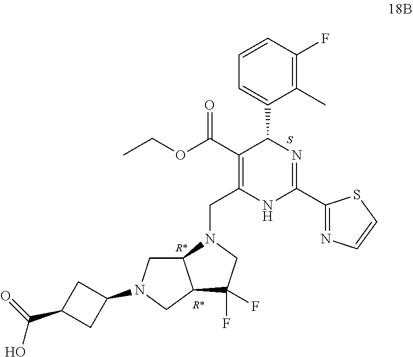

This compound was made from T6B analogous to 18A. Purified by C18 column (acetonitrile:water (add 0.02% ammonium bicarbonate)=35% to 45%) to give the title compound (15 mg, 97.5% purity, 38% yield) as yellow solids. LC-MS (ESI): mass calcd. for C₂₉H₃₂F₃N₅O₄S 603.6, m/z found 604.3 [M+H]⁺. ¹H NMR (400 MHz, CDCl₃+D₂O) δ 7.71 (d, J=2.8 Hz, 1H), 7.29 (d, J=2.0 Hz, 1H), 7.14-7.08 (m, 1H), 7.03-7.01 (m, 1H), 6.95-6.91 (m, 1H), 6.00 (s, 1H), 4.30 (d, J=16.4 Hz, 1H), 4.07-4.02 (m, 3H), 3.82-3.79 (m, 1H), 3.41-3.19 (m, 5H), 2.96-2.79 (m, 4H), 2.53 (s, 3H), 2.49-2.45 (m, 4H), 1.11 (t, J=7.2 Hz, 3H).

### Compound 19B: 3-((cis)-3,3-difluoro-1-((6-(3-fluoro-2-methylphenyl)-5-(methoxycarbonyl)-2-(thiazol-2-yl)-3,6-dihydropyrimidin-4-yl)methyl)hexahydropyrrolo[3,4-b]pyrrol-5(1H)-yl)-2,2-dimethylpropanoic acid (Single Diastereomer)

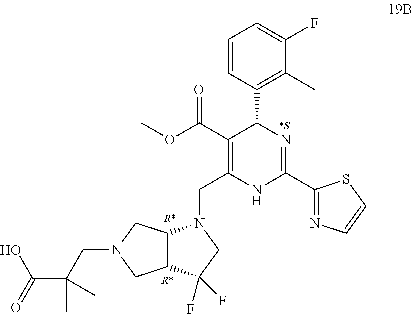

This compound was made according to Typical coupling method 1 from H4-1B with T2. Purified by C18 column (acetonitrile:water=30% to 50%) to give the title compound (30 mg, 98.1% purity, 56% yield) as yellow solid. LC-MS (ESI): mass calcd. for C₂₈H₃₂F₃N₅O₄S 591.6, m/z found 592.3 [M+H]⁺. ¹H NMR (400 MHz, CDCl₃) δ 9.17 (s, 1H), 7.87 (d, J=2.8 Hz, 1H), 7.40 (d, J=3.2 Hz, 1H), 7.11-7.05 (m, 1H), 6.99-6.97 (m, 1H), 6.93-6.89 (m, 1H), 6.00 (s, 1H), 4.35 (d, J=16.8 Hz, 1H), 4.06 (d, J=16.0 Hz, 1H), 3.75-3.72 (m, 1H), 3.59 (s, 3H), 3.51 (d, J=6.8 Hz, 1H), 3.40-3.34 (m, 2H), 3.11-3.03 (m, 1H), 2.98-2.89 (m, 1H), 2.72 (d, J=13.6 Hz, 1H), 2.68 (d, J=13.6 Hz, 1H), 2.61-2.57 (m, 2H), 2.53 (s, 1.5H), 2.52 (s, 1.5H), 1.26 (s, 3H), 1.23 (s, 3H).

### Compound 20B: 3-((cis)-1-((6-(2-chloro-4-fluorophenyl)-5-(methoxycarbonyl)-2-(thiazol-2-yl)-3,6-dihydropyrimidin-4-yl)methyl)-3,3-difluorohexahydropyrrolo[3,4-b]pyrrol-5(1H)-yl)-2,2-dimethylpropanoic acid (Single Diastereomer)

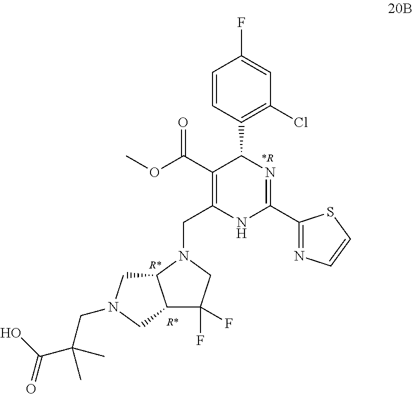 [SMILES: COC(=O)C1=C(CN2CC(F)(F)[C@@H]3CN(CC(C)(C)C(=O)O)C[C@@H]32)NC(c2nccs2)=N[C@H]1c1ccc(F)cc1Cl]

This compound was made according to Typical coupling method 1 from H3-1A with T2. Purified by Prep. HPLC (Column: Xbridge C18 (5 μm 19*150 mm), Mobile Phase A: water (+0.1% ammonium bicarbonate), Mobile Phase B: acetonitrile, UV: 254 nm, Flow rate: 15 mL/min, Gradient: 30-60% (% B)) to give the title compound (45.5 mg, 95.9% purity, 67% yield, 99.3% stereopure) as yellow solid. LC-MS (ESI): R_{T}=3.727 min, mass calcd. for C₂₇H₂₉ClF₃N₅O₄S 611.2, m/z found 612.3 [M+H]⁺. Chiral analysis (Column: Chiralpark column: IE 5 μm 4.6*250 mm; Mobile Phase: Hex:IPA:TFA:DEA=80:20:0.1:0.1 at 1 mL/min; Wavelength: 254 nm, R_{T}=12.299 min). ¹H NMR (400 MHz, CD₃OD) δ 7.98 (d, J=3.2 Hz, 1H), 7.78 (s, 1H), 7.51-7.47 (m, 1H), 7.29 (dd, J=8.8, 2.8 Hz, 1H), 7.14-7.10 (m, 1H), 6.20 (s, 1H), 4.31-4.21 (m, 2H), 3.96-3.89 (m, 1H), 3.65 (s, 3H), 3.57-3.48 (m, 2H), 3.25-3.12 (m, 3H), 2.96-2.72 (m, 4H), 1.28 (s, 3H), 1.27 (s, 3H).

### Compound 21B: 3-((cis)-1-((6-(2-chloro-3,4-difluorophenyl)-5-(methoxycarbonyl)-2-(thiazol-2-yl)-3,6-dihydropyrimidin-4-yl)methyl)-3,3-difluorohexahydropyrrolo[3,4-b]pyrrol-5(1H)-yl)-2,2-dimethylpropanoic acid (Single Diastereomer)

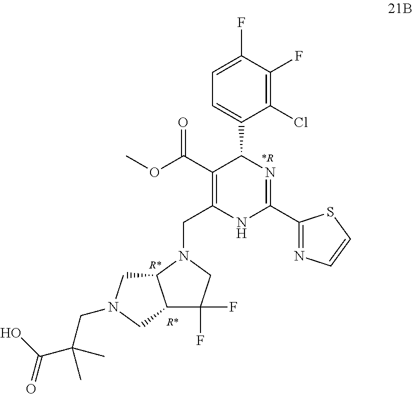 [SMILES: COC(=O)C1=C(CN2CC(F)(F)[C@@H]3CN(CC(C)(C)C(=O)O)C[C@@H]32)NC(c2nccs2)=N[C@H]1c1ccc(F)c(F)c1Cl]

This compound was made according to Typical coupling method 1 from H5-1A with T2. Purified by C18 column (acetonitrile:water=30% to 95%) to give the title compound (18 mg, 97% purity, 25% yield) as yellow solids. LC-MS (ESI): mass calcd. for C₂₇H₂₈ClF₄N₅O₄S 629.1, m/z found 630.3 [M+H]⁺. ¹H NMR (400 MHz, CD₃OD) δ 7.94-7.93 (m, 1H), 7.74 (br s, 1H), 7.26-7.24 (m, 2H), 6.16 (s, 1H), 4.33-4.14 (m, 2H), 3.93-3.86 (m, 1H), 3.61 (s, 3H), 3.56-3.41 (m, 2.5H), 3.23-2.67 (m, 6.5H), 1.24 (s, 3H), 1.23 (s, 3H).

### Compound 22B: 3-((cis)-1-((6-(2-chloro-3-fluorophenyl)-5-(ethoxycarbonyl)-2-(thiazol-2-yl)-3,6-dihydropyrimidin-4-yl)methyl)-3,3-difluorohexahydropyrrolo[3,4-b]pyrrol-5(1H)-yl)-2,2-dimethylpropanoic acid (Single Diastereomer)

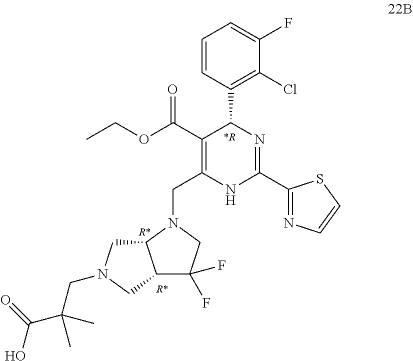 [SMILES: CCOC(=O)C1=C(CN2CC(F)(F)[C@@H]3CN(CC(C)(C)C(=O)O)C[C@@H]32)NC(c2nccs2)=N[C@H]1c1cccc(F)c1Cl]

This compound was made according to Typical coupling method 1 from H1-1A with T2. Purified by C18 column (acetonitrile:water (0.1% ammonium bicarbonate)=45% to 55%) to afford the title compound (60 mg, 98.3% purity, 49% yield) as yellow solids. LC-MS (ESI): mass calcd. for C₂₈H₃₁F₃N₅O₄S 625.2, m/z found 626.3 [M+H]⁺. Chiral analysis (Column: Chiralpark column: IG 5 μm 4.6*250 mm; Mobile Phase: Hex:EtOH:TFA=85:15:0.2 at 1 mL/min; Wavelength: 254 nm, R_{T}=9.191 min). ¹H NMR (400 MHz, CD₃OD) δ 7.94 (d, J=3.2 Hz, 1H), 7.74 (d, J=2.4 Hz, 1H), 7.36-7.26 (m, 2H), 7.19-7.15 (m, 1H), 6.23 (s, 1H), 4.36-4.18 (m, 2H), 4.05 (q, J=7.2 Hz, 2H), 3.94-3.85 (m, 1H), 3.71-3.36 (m, 3H), 3.28-2.68 (m, 6H), 1.24 (s, 3H), 1.23 (s, 3H), 1.13 (t, J=7.2 Hz, 3H).

### Preparation of Intermediate T15

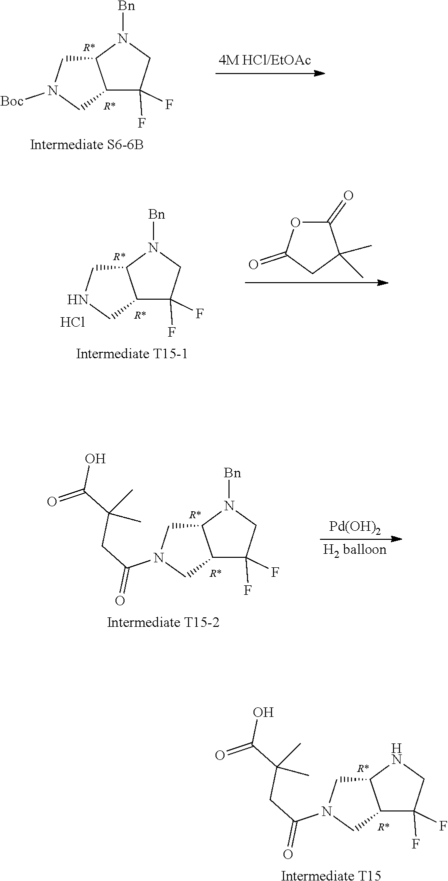

### T15-1: (cis)-1-Benzyl-3,3-difluorooctahydropyrrolo[3,4-b]pyrrole hydrochloride

A solution of S6-6B (1.00 g, 90% purity, 2.66 mmol) in 4 M hydrochloride in ethyl acetate (15 mL) was stirred at 15° C. for 1 hour. The reaction mixture was concentrated in vacuo to give the title compound (760 mg, 90% purity from ¹H NMR, 94% yield) as brown solids. ¹H NMR (400 MHz, DMSO-d₆) δ 10.27 (s, 1H), 9.14 (s, 1H), 7.43 (d, J=4.0 Hz, 2H), 7.38-7.29 (m, 3H), 4.10-4.07 (m, 1H), 3.85 (br s, 1H), 3.62-3.59 (m, 1H), 3.53-3.44 (m, 2H), 3.39-3.23 (m, 3H), 3.07-2.84 (m, 2H).

### T15-2: 4-((cis)-1-Benzyl-3,3-difluorohexahydropyrrolo[3,4-b]pyrrol-5(1H)-yl)-2,2-dimethyl-4-oxobutanoic acid

The solution of T15-1 (120 mg, 90% purity, 0.390 mmol), triethylamine (119 mg, 1.17 mmol) and 3,3-dimethyldihydrofuran-2,5-dione (100 mg, 0.780 mmol) in tetrahydrofuran (8 mL) was stirred at room temperature overnight under nitrogen atmosphere. Then the reaction mixture was poured into water (20 mL) and extracted with ethyl acetate (20 mL) twice. The combined organic phases were washed with brine (20 mL), dried over Na₂SO₄₍ₛ₎ and filtered. The filtrate was concentrated and purified by C18 column (acetonitrile:water=30% to 95%) to give the title compound (100 mg, 90% purity from ¹H NMR, 69% yield) as colorless oil. ¹H NMR (400 MHz, DMSO-d₆) δ 7.35-7.24 (m, 5H), 3.96-3.85 (m, 1H), 3.68-3.61 (m, 2H), 3.58-3.43 (m, 2H), 3.40-3.34 (m, 3H), 3.21-3.13 (m, 2H), 2.93-2.75 (m, 2H), 1.16-1.14 (m, 6H).

### T15: 4-((cis)-3,3-Difluorohexahydropyrrolo[3,4-b]pyrrol-5(1H)-yl)-2,2-dimethyl-4-oxobutanoic acid

To a mixture of T15-2 (80 mg, 90% purity, 0.197 mmol) in isopropyl alcohol (7 mL) was added palladium hydroxide (40 mg, 20% purity, 0.05 mmol). Then, the mixture was stirred at 20° C. under hydrogen atmosphere (balloon) overnight. The catalyst was filtered off and washed with 10 mL mixed solution of methanol/water (v/v=10/1). The filtrate was concentrated under reduced pressure to give the title compound (60 mg, 90% purity from ¹H NMR, 99% yield) as brown oil. ¹H NMR (400 MHz, CDCl₃) δ 5.30-5.25 (m, 2H), 4.20-4.09 (m, 1H), 3.89-3.79 (m, 1H), 3.76-3.66 (m, 1H), 3.64-3.51 (m, 2H), 3.34-3.25 (m, 2H), 3.09-2.96 (m, 1H), 2.65-2.44 (m, 2H), 1.29-1.20 (m, 6H).

### Compound 23B: 4-((cis)-1-(((S)-5-(Ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)-2,2-dimethyl-4-oxobutanoic acid (Single Diastereomer)

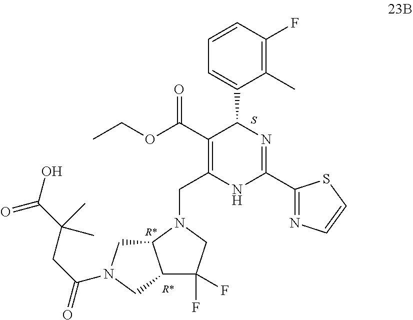

This compound was made according to Typical coupling method 1 from H2-1A with T15. Purified by C18 column (acetonitrile:water=30% to 95%) to give the title compound (15 mg, 95% purity, 11% yield) as yellow solids. LC-MS (ESI): mass calcd. for C₃₀H₃₄F₃N₅O₅S 633.2, m/z found 634.3 [M+H]⁺. ¹H NMR (400 MHz, CDCl₃) δ 9.19 (s, 0.6H), 8.89 (s, 0.4H), 7.88-7.84 (m, 1H), 7.44-7.42 (m, 1H), 7.17-7.00 (m, 2H), 6.91-6.89 (m, 1H), 6.01 (s, 0.6H), 6.00 (s, 0.4H), 4.59-4.55 (m, 0.5H), 4.43-4.39 (m, 0.5H), 4.12-3.94 (m, 4H), 3.85-3.65 (m, 3H), 3.54-3.38 (m, 2H), 3.26-2.89 (m, 2H), 2.73-2.69 (m, 1H), 2.53 (d, J=4.0 Hz, 3H), 2.47-2.40 (m, 0.5H), 2.25-2.21 (m, 0.5H), 1.44 (s, 1.5H), 1.34 (s, 1.5H), 1.21-1.20 (m, 3H), 1.13-1.09 (m, 3H).

### Compound 24A: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3a-fluoro-6-oxohexahydropyrrolo[3,4-b]pyrrol-5(1H)-yl)-2,2-dimethylpropanoic acid

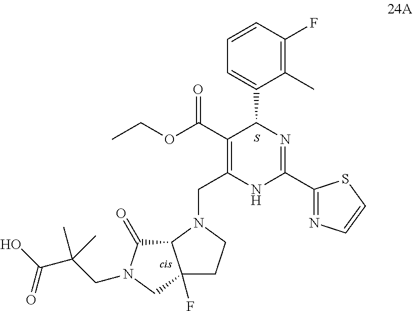

This compound is made from S4 and H2-1A according to typical method 1. LC-MS (ESI): mass calcd. for C₂₉H₃₃F₂N₅O₅S 601.2, m/z found 602.2 [M+H]⁺. ¹H NMR (400 MHz, CDCl₃) δ 9.01 (s, 1H), 7.88 (d, J=3.2 Hz, 1H), 7.40 (d, J=2.8 Hz, 1H), 7.12-7.06 (m, 2H), 6.92-6.87 (m, 1H), 6.01 (s, 1H), 4.77 (d, J=16.4 Hz, 1H), 4.24 (d, J=16.1 Hz, 1H), 4.07-4.02 (m, 3H), 3.85 (t, J=10.0 Hz, 1H), 3.74-3.66 (m, 1H), 3.57 (d, J=26.8 Hz, 1H), 3.05 (t, J=8.0 Hz, 1H), 2.92 (d, J=14.0 Hz, 1H), 2.85-2.78 (m, 1H), 2.52 (d, J=1.6 Hz, 3H), 2.46-2.35 (m, 1H), 2.27-2.11 (m, 1H), 1.36 (s, 3H), 1.30 (s, 3H), 1.11 (t, J=7.2 Hz, 3H).

### Preparation of Intermediate S39

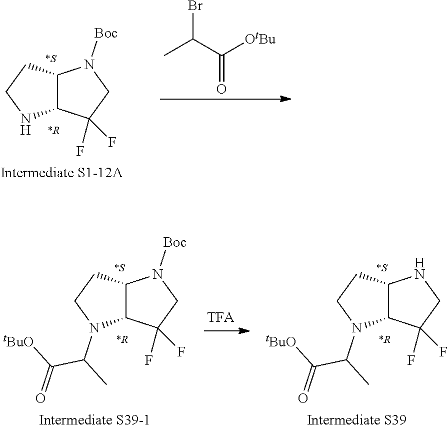

### S39-1: (cis)-tert-Butyl 4-(1-(tert-butoxy)-1-oxopropan-2-yl)-3,3-difluorohexahydropyrrolo[3,2-b]pyrrole-1(2H)-carboxylate

To a solution of S1-12A (300 mg, 95% purity, 1.00 mmol) in N,N-dimethylformamide (5 mL) was added tert-butyl 2-bromopropanoate (513 mg, 2.45 mmol) and N-ethyl-N-isopropylpropan-2-amine (645 mg, 4.99 mmol). After stirred at 50° C. overnight, the mixture was poured into water (30 mL) and extracted with ethyl acetate (30 mL) for three times. The combined organic layers were washed with brine (50 mL), dried over Na₂SO₄₍ₛ₎, filtered and concentrated to give a residue, which was purified by silica gel column chromatography (petroleum ether:ethyl acetate=5:1) to give the desired compound (220 mg, 100% purity, 58% yield) as pale yellow oil. LC-MS (ESI): mass calcd. for C₁₈H₃₀F₂N₂O₄ 376.4, m/z found 377.4 [M+H]⁺.

S39: tert-Butyl 2-((cis)-6,6-difluorohexahydropyrrolo[3,2-b]pyrrol-1(2H)-yl)propanoate

To a solution of S39-1 (220 mg, 100% purity, 0.584 mmol) in dichloromethane (20 mL) was added trifluoroacetic acid (2 mL). The mixture was stirred at room temperature for 4 hours. Then it was concentrated to give a residue, which was basified with saturated sodium bicarbonate aqueous solution to pH=8 and extracted with ethyl acetate (30 mL) for three times. The combined organic layers were washed with brine (30 mL), dried over Na₂SO₄₍ₛ₎, filtered and concentrated to give a the title compound (146 mg, 66% purity, 60% yield) as pale yellow solids. LC-MS (ESI): mass calcd. for C₁₃H₂₂F₂N₂O₂ 276.3, m/z found 277.5 [M+H]⁺.

### Compound 26-M: (4S)-Ethyl 6-(((cis)-4-(1-(tert-butoxy)-1-oxopropan-2-yl)-3,3-difluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

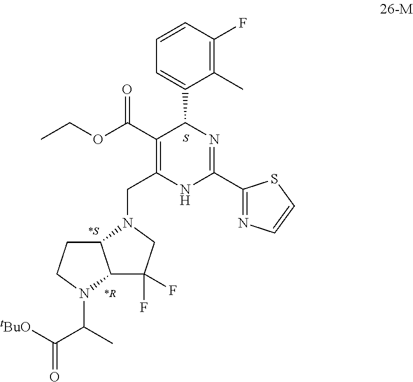

Compound 26-M was made from H2-1A and S39 according to typical coupling method 1. Purified by C18 column (acetonitrile:water=70% to 95%) to give the desired compound (110 mg, 100% purity, 50% yield) as yellow solids. LC-MS (ESI): mass calcd. for C₃₁H₃₈F₃N₅O₄S 633.7, m/z found 634.7 [M+H]⁺.

Compound 26-M (290 mg, 90% purity, 0.412 mmol) was separated by chiral Prep. HPLC (Column: Chiralpak IE 5 μm 20*250 mm; Mobile Phase: Hex:EtOH=95:5 at 30 mL/min; Temp: 30° C.; Wavelength: 254 nm) to give the title compounds 26a (110 mg, 90% purity, 38% yield, 100% stereopure) and 26b (111 mg, 90% purity, 38% yield, 100% stereopure) as colorless oil.

26a: Chiral analysis (Column: Chiralpak IE 5 μm 4.6*250 mm; Mobile Phase: Hex:EtOH=95:5 at 1.0 mL/min; Temp: 30° C.; Wavelength: 230 nm, R_{T}=7.709 min).

26b: Chiral analysis (Column: Chiralpak IE 5 μm 4.6*250 mm; Mobile Phase: Hex:EtOH=95:5 at 1.0 mL/min; Temp: 30° C.; Wavelength: 230 nm, R_{T}=8.992 min).

### Compound 26E and 26F: 2-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)propanoic acid

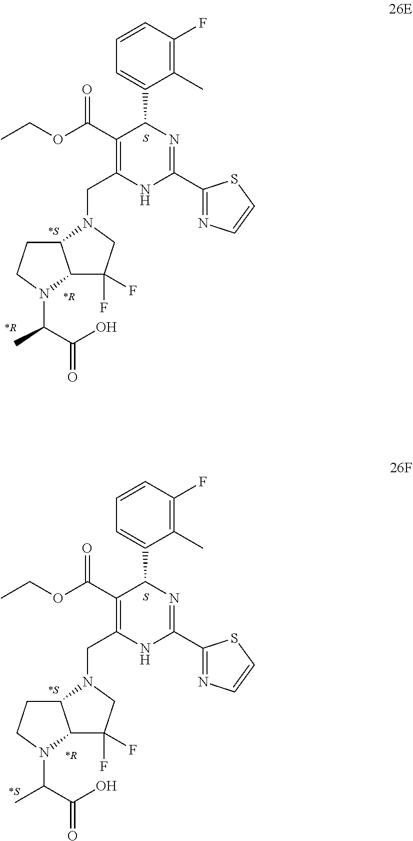

Compound 26E and 26F were made from 26a and 26b according to typical method 3, respectively.

Compound 26E: purified by C18 column (acetonitrile:water=40% to 75%) to give the desired compound (59.9 mg, 98% purity, 65% yield) as yellow solids. LC-MS (ESI): mass calcd. for C₂₇H₃₀F₃N₅O₄S 577.6, m/z found 578.3 [M+H]⁺. ¹H NMR (400 MHz, CDCl₃) δ 9.35 (s, 1H), 7.84 (d, J=3.2 Hz, 1H), 7.40 (d, J=2.8 Hz, 1H), 7.10-7.05 (m, 1H), 7.00-6.98 (m, 1H), 6.92-6.88 (m, 1H), 6.01 (s, 1H), 4.29-4.24 (m, 1H), 4.09-3.99 (m, 3H), 3.88-3.85 (m, 1H), 3.77-3.72 (m, 1H), 3.66-3.60 (m, 1H), 3.38-3.30 (m, 3H), 3.02-2.94 (m, 1H) 2.91-2.85 (m, 1H), 2.53 (s, 3H), 2.05-1.91 (m, 2H), 1.46 (d, J=7.2 Hz, 3H), 1.11 (t, J=7.2 Hz, 3H).

Compound 26F: purified by C18 column (acetonitrile:water=40% to 75%) to give the desired compound (65.6 mg, 99% purity, 71% yield) as yellow solids. LC-MS (ESI): mass calcd. for C₂₇H₃₀F₃N₅O₄S 577.6, m/z found 578.3 [M+H]⁺. ¹H NMR (400 MHz, CDCl₃) δ 9.37 (s, 1H), 7.87 (d, J=3.2 Hz, 1H), 7.41 (d, J=2.8 Hz, 1H), 7.10-7.06 (m, 1H), 7.00-6.98 (m, 1H), 6.93-6.89 (m, 1H), 6.01 (s, 1H), 4.31-4.26 (m, 1H), 4.07-3.99 (m, 3H), 3.89-3.85 (m, 1H), 3.78-3.67 (m, 2H), 3.42-3.34 (m, 1H), 3.17-2.89 (m, 4H) 2.53 (s, 3H), 2.07-2.00 (m, 2H), 1.38 (d, J=7.2 Hz, 3H), 1.11 (t, J=7.2 Hz, 3H).

### Compound 28: 3-((cis)-1-((6-(2-chloro-4-fluorophenyl)-5-(methoxycarbonyl)-2-(thiazol-2-yl)-3,6-dihydropyrimidin-4-yl)methyl)-3,3-difluoro-4-oxohexahydropyrrolo[3,4-b]pyrrol-5(1H)-yl)-2,2-dimethylpropanoic acid (Single Diastereomer)

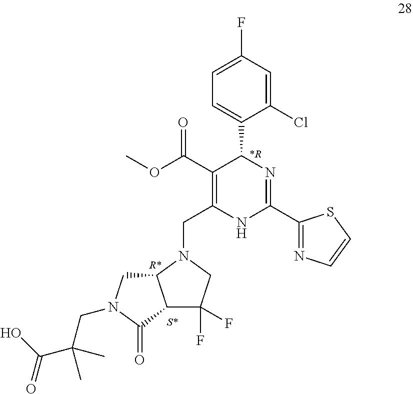 [SMILES: COC(=O)C1=C(CN2CC(F)(F)[C@@H]3C(=O)N(CC(C)(C)C(=O)O)C[C@@H]32)NC(c2nccs2)=N[C@H]1c1ccc(F)cc1Cl]

This compound was made using the procedure similar to Compound 42 by replacing H5-1A with H3-1A. Purified by C18 column (acetonitrile:water (0.02% ammonium bicarbonate)=05% to 70%) to give the title compound (60 mg, 99.8% purity) as yellow solids. LC-MS (ESI): mass calcd. for C₂₇H₂₇ClF₃N₅O₅S 625.1, m/z found 626.2 [M+H]⁺. ¹H NMR (400 MHz, CDCl₃) δ 9.24 (br s, 1H), 7.83 (d, J=3.2 Hz, 1H), 7.46 (d, J=2.8 Hz, 1H), 7.34 (dd, J=8.8, 6.0 Hz, 1H), 7.13 (dd, J=7.6, 2.8 Hz, 1H), 6.94 (td, J=8.4, 2.8 Hz, 1H), 6.19 (s, 1H), 4.51-4.47 (m, 1H), 4.07-4.04 (m, 1H), 3.84-3.82 (m, 2H), 3.62-3.59 (m, 4H), 3.43-3.30 (m, 3H), 2.96-2.75 (m, 2H), 1.38 (s, 3H), 1.35 (s, 3H).

### Compound 29: 3-((cis)-1-((6-(2-chloro-3-fluorophenyl)-5-(methoxycarbonyl)-2-(thiazol-2-yl)-3,6-dihydropyrimidin-4-yl)methyl)-3,3-difluoro-4-oxohexahydropyrrolo[3,4-b]pyrrol-5(1H)-yl)-2,2-dimethylpropanoic acid (Single Diastereomer)

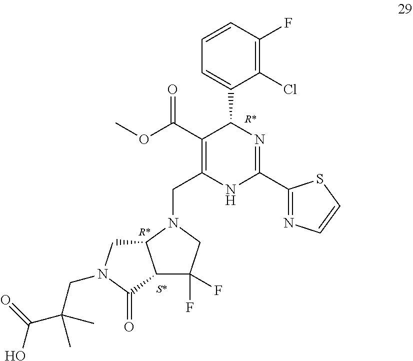 [SMILES: COC(=O)C1=C(CN2CC(F)(F)[C@@H]3C(=O)N(CC(C)(C)C(=O)O)C[C@@H]32)NC(c2nccs2)=N[C@H]1c1cccc(F)c1Cl]

This compound was made using the procedure similar to Compound 42 by replacing H5-1A with H11-1A. Purified by C18 column (acetonitrile:water (0.1% ammonium bicarbonate=5% to 60%) to give the product (55 mg, 99.4% purity) as yellow solids. LC-MS (ESI): R_{T}=3.166 min, mass calcd. for C₂₇H₂₇ClF₃N₅O₅S 625.1, m/z found 626.2 [M+H]⁺. ¹H NMR (400 MHz, CDCl₃) δ 9.27 (s, 1H), 7.82 (d, J=3.2 Hz, 1H), 7.45 (d, J=3.2 Hz, 1H), 7.22-7.13 (m, 2H), 7.09-7.00 (m, 1H), 6.26 (s, 1H), 4.51 (d, J=15.6 Hz, 1H), 4.01 (d, J=13.6 Hz, 1H), 3.85-3.79 (m, 2H), 3.65-3.61 (m, 1H), 3.60 (s, 3H), 3.47-3.43 (m, 1H), 3.41-3.33 (m, 1H), 3.32-3.25 (m, 1H), 2.99 (d, J=14.0 Hz, 1H), 2.86-2.76 (m, 1H), 1.36 (s, 3H), 1.33 (s, 3H).

### Compound 30: trans-4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorooctahydropyrrolo[3,2-b]pyrrole-1-carbonyl)cyclohexane-1-carboxylic acid

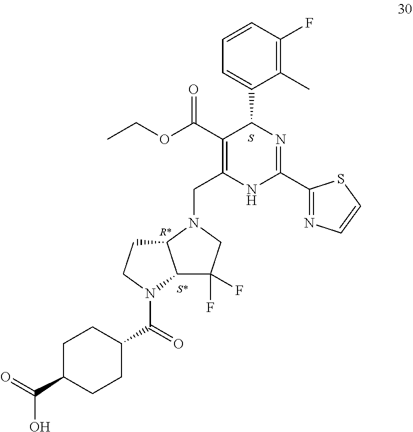

### Preparation of Intermediate S50

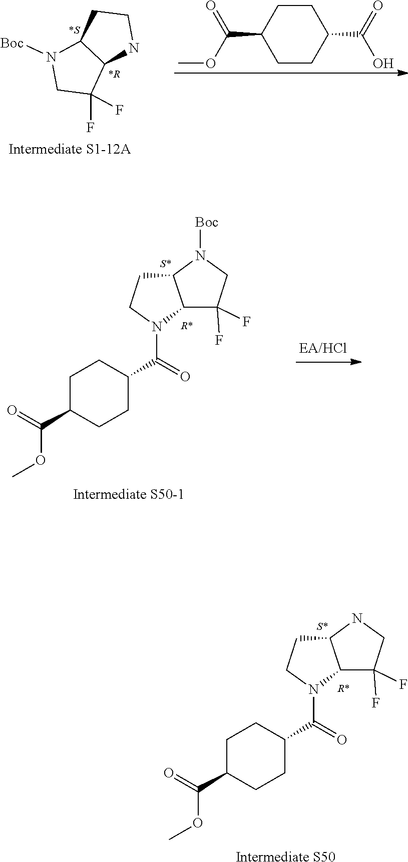

### S50-1: (cis)-tert-Butyl 3,3-difluoro-4-((trans)-4-(methoxycarbonyl)cyclohexanecarbonyl)hexahydropyrrolo[3,2-b]pyrrole-1(2H)-carboxylate

To a solution of S1-12A (130 mg, 80% purity, 0.419 mmol) and (trans)-4-(methoxycarbonyl)cyclohexanecarboxylic acid (90 mg, 0.483 mmol) in N,N-dimethylformamide (6 mL) was added N,N-diisopropylethylamine (108 mg, 0.836 mmol) and 2-(3H-[1,2,3]triazolo[4,5-b]pyridin-3-yl)-1,1,3,3-tetramethylisouronium hexafluoro-phosphate(V) (319 mg, 0.839 mmol). After stirred at room temperature overnight under nitrogen, the mixture was added ethyl acetate (50 mL). The organic layer was washed with water (10 mL) twice and brine (10 mL), dried over sodium sulfate(s), filtered and concentrated. The residue was purified by C18 column (acetonitrile:water=20% to 80%) to afford the desired product (160 mg, 80% purity, 73% yield) as white solids. LC-MS (ESI): mass calcd. for C₂₀H₃₀F₂N₂O₅ 416.2, m/z found 417.4 [M+H]⁺.

### S50: (trans)-Methyl 4-((cis)-6,6-difluorooctahydropyrrolo[3,2-b]pyrrole-1-carbonyl)cyclohexanecarboxylate

To a solution of S50-1 (160 mg, 80% purity, 0.307 mmol) in ethyl acetate (2 mL) was added 4 M hydrochloride in ethyl acetate (2 mL, 8 mmol). After stirred at room temperature for 1 hour, the mixture was concentrated to give a residue, which was diluted with ethyl acetate (50 mL). The organic solution was washed with saturated sodium carbonate solution (10 mL) for three times and brine (10 mL), dried over sodium sulfate(s), filtered and concentrated to afford the desired product (110 mg, 97% yield, 86% purity) as colorless oil. LC-MS (ESI): mass calcd. for C₁₅H₂₂F₂N₂O₃ 316.2, m/z found 317.3 [M+H]⁺.

### Compound 31: (S)-Ethyl 6-(((cis)-3,3-difluoro-5-((methylsulfonyl)carbamoyl)-hexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate (Single Diastereomer)

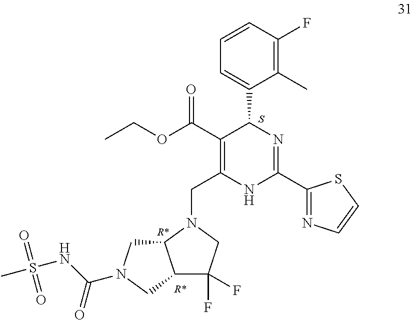

To a solution of 4-nitrophenyl carbonochloridate (81 mg, 0.40 mmol) and 4-dimethylaminopyridine (50 mg, 0.41 mmol) in dichloromethane (5 mL) was added triethylamine (81 mg, 0.80 mmol) and methanesulfonamide (40 mg, 0.42) at room temperature. The mixture was stirred at room temperature for 2 hours before Compound 179 (170 mg, 95% purity, 0.319 mmol) was added. After stirred at 30° C. under nitrogen atmosphere overnight, the reaction mixture was cooled to room temperature, diluted with water (10 mL) and extracted with ethyl acetate (10 mL) twice. The combined organic layers were washed with brine (10 mL), dried over Na₂SO₄₍ₛ₎, filtered and concentrated to give a residue, which was purified by C18 column (acetonitrile:water=40% to 70%) to give the title compound (35.5 mg, 95.2% purity, 13% yield) as yellow solids. LC-MS (ESI): mass calcd. for C₂₆H₂₉F₃N₆O₅S₂ 626.2, m/z found 627.2. ¹H NMR (400 MHz, CD₃OD) δ 7.98 (d, J=3.2 Hz, 1H), 7.73 (d, J=3.2 Hz, 1H), 7.25-7.14 (m, 2H), 6.97-6.92 (m, 1H), 5.98 (s, 1H), 4.42-4.30 (m, 1H), 4.15-4.02 (m, 4H), 3.98-3.81 (m, 2H), 3.76-3.60 (m, 1H), 3.59-3.49 (m, 2H), 3.28-3.02 (m, 2H), 2.98 (s, 3H), 2.51 (s, 3H), 1.15 (t, J=6.8 Hz, 3H).

### Compound 32: ethyl (S)-6-(((cis)-5-(N-acetylsulfamoyl)-3,3-difluorohexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate (Single Diastereomer)

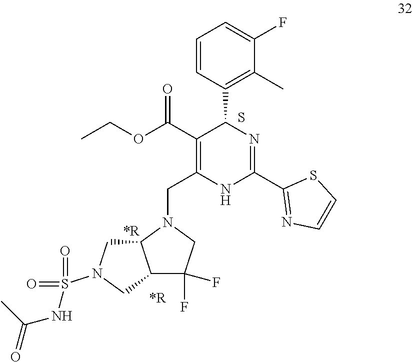

To a mixture of Compound 33 (37 mg, 97% purity, 0.061 mmol) and triethylamine (280 mg, 2.77 mmol) in dichloromethane (2 mL) was added a solution of acetyl chloride (50 mg, 0.637 mmol) in dichloromethane (0.5 mL) at 0° C. After stirred at 0° C. for 0.5 hour, the mixture was concentrated under reduced pressure to give a residue, which was purified by C18 column (acetonitrile:water (add 0.02% ammonium bicarbonate)=5% to 95%) to give the title compound (17 mg, 99.3% purity, 44% yield) as yellow solids. LC-MS (ESI): mass calcd. for C₂₆H₂₉F₃N₆O₅S₂ 626.2, m/z found 627.1 [M+H]⁺. ¹H NMR (400 MHz, CDCl₃) δ 9.13 (s, 0.7H), 7.84 (d, J=3.2 Hz, 1H), 7.54-7.52 (m, 0.2H), 7.43 (d, J=3.2 Hz, 0.8H), 7.22-7.06 (m, 3H), 6.93-6.89 (m, 1.3H), 6.02 (s, 0.9H), 5.97 (s, 0.1H), 4.37 (d, J=17.2 Hz, 0.8H), 4.18-4.14 (m, 0.2H), 4.08-4.02 (m, 4H), 3.94-3.89 (m, 1H), 3.81-3.72 (m, 2H), 3.62-3.58 (m, 1H), 3.51-3.42 (m, 1H), 3.22-3.15 (m, 1H), 3.00-2.90 (m, 1H), 2.54 (d, J=1.6 Hz, 2.7H), 2.40 (s, 0.3H), 2.05 (s, 0.4H), 1.87 (s, 2.6H), 1.14-1.07 (m, 3H).

### Compound 33: ethyl (S)-6-(((cis)-3,3-difluoro-5-sulfamoylhexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate (Single Diastereomer)

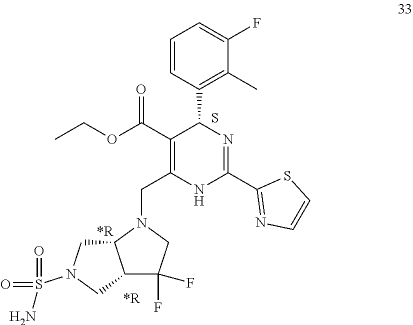

This compound was made using the procedure similar to Compound 46 by replacing tert-butyl 3-(chlorosulfonyl)-2,2-dimethylpropanoate with tert-butyl (chlorosulfonyl)carbamate. Purified by C18 column (acetonitrile:water (add 0.02% ammonium bicarbonate)=5% to 95%) to give the title compound (25 mg, 99.0% purity) as yellow solid. LC-MS (ESI): mass calcd. for C₂₄H₂₇F₃N₆O₄S₂ 584.2, m/z found 585.2 [M+H]⁺. ¹H NMR (400 MHz, CDCl₃) δ 9.07 (s, 0.7H), 7.83 (d, J=3.6 Hz, 1H), 7.55 (br s, 0.1H), 7.45 (d, J=3.2 Hz, 0.9H), 7.21 (br s, 0.3H), 7.13-7.07 (m, 1H), 6.99-6.89 (m, 2H), 6.02 (s, 0.9H), 5.93 (s, 0.1H), 5.44 (s, 0.3H), 5.24 (s, 1.7H), 4.54 (d, J=16.8 Hz, 1H), 4.06-4.01 (m, 2H), 3.94-3.88 (m, 2H), 3.80-3.78 (m, 1H), 3.68-3.65 (m, 1H), 3.53-3.33 (m, 3H), 3.18-3.13 (m, 1H), 3.02-2.93 (m, 1H), 2.53 (s, 2.7H), 2.39 (s, 0.3H), 1.11 (t, J=7.2 Hz, 3H).

### Compound 34: (S)-Ethyl 6-(((cis)-3,3-difluoro-4-sulfamoylhexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

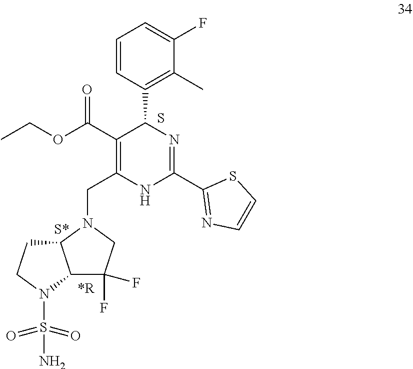

### Step 1: (S)-Ethyl 6-(((cis)-4-(N-(tert-butoxycarbonyl)sulfamoyl)-3,3-difluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

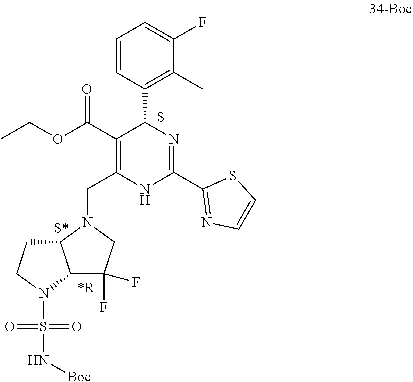

To the solution of compound 103 (80 mg, 90% purity, 0.14 mmol) and tert-butyl chlorosulfonylcarbamate (45 mg, 0.17 mmol) in dichloromethane (2 mL) was added triethylamine (30 mg, 0.30 mmol) at room temperature under nitrogen atmosphere. After stirring at room temperature overnight under nitrogen atmosphere, the mixture was concentrated under reduced pressure to give a residue, which was diluted in ethyl acetate (30 mL) and washed with water (15 mL) twice. The combined aqueous layers were extracted with ethyl acetate (20 mL) twice. The combined organic layers were washed with water (10 mL) twice and brine (10 mL), dried over Na₂SO₄₍ₛ₎ and filtered. The filtrate was concentrated under reduced pressure to afford the residue, which was purified by C18 column (acetonitrile water=40% to 80%) to give the title compound (90 mg, 87% purity, 81% yield) as yellow solids. LC-MS (ESI): mass calcd. for C₂₉H₃₅F₃N₆O₆S₂ 684.8, m/z found 685.7 [M+H]⁺.

Step 2: compound 34-Boc (90 mg, 87% purity, 0.11 mmol) in dichloromethane (2 mL) was added trifluoroacetic acid (1 mL) at 0° C. After stirred at room temperature for 1 hour, the mixture was concentrated under reduced pressure to give a residue, which was diluted in ethyl acetate (30 mL) and washed with water (15 mL) twice. The combined aqueous layers were extracted with ethyl acetate (20 mL) twice. The combined organic layers were washed with water (10 mL) twice and brine (10 mL), dried over Na₂SO₄₍ₛ₎ and filtered. The filtrate was concentrated under reduced pressure to afford a residue, which was purified by C18 column (acetonitrile:water=35% to 55%) to give the title compound (25.5 mg, 99.0% purity, 37% yield) as yellow solids. LC-MS (ESI): mass calcd. for C₂₄H₂₇F₃N₆O₄S₂ 584.6, m/z found 585.2 [M+H]⁺. ¹H NMR (400 MHz, DMSO-d₆) δ 9.62 (s, 0.2H), 9.31 (s, 0.8H), 8.01-7.99 (m, 1H), 7.92-7.91 (m, 1H), 7.20-7.15 (m, 1H), 7.07-7.01 (m, 2H), 6.91 (s, 1.5H), 6.81 (s, 0.5H), 5.89 (s, 0.8H), 5.77 (s, 0.2H), 4.42-4.36 (m, 1H), 4.20 (d, J=16.8 Hz, 1H), 4.09 (d, J=16.8 Hz, 1H), 4.00-3.95 (m, 2H), 3.85-3.82 (m, 1H), 3.61-3.59 (m, 1H), 3.29 (s, 2H), 3.05-2.95 (m, 1H), 2.44 (s, 2.4H), 2.40 (s, 0.6H), 2.00-1.96 (m, 1H), 1.89-1.83 (m, 1H), 1.08-1.02 (m, 3H).

### 37E and 37F: 3-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)-2-methylpropanoic acid

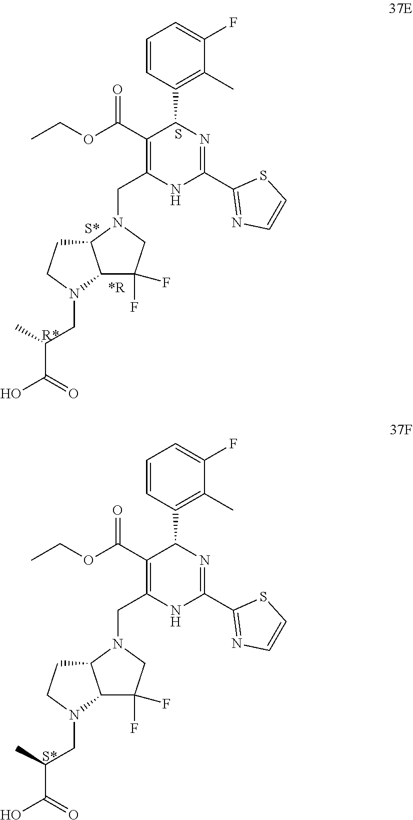

Compound 37E and 37F were made from H2-1A and S43-A and S43B, respectively, according to typical method 1 and 3 successively.

Compound 37E: LC-MS (ESI): mass calcd. for C₂₈H₃₂F₃N₅O₄S 591.2, m/z found 592.3 [M+H]⁺. ¹H NMR (400 MHz, CDCl₃) δ 9.22 (s, 1H), 7.83 (d, J=3.2 Hz, 1H), 7.41 (d, J=2.8 Hz, 1H), 7.07-7.04 (m, 1H), 6.98 (d, J=6.8 Hz, 1H), 6.93-6.88 (m, 1H), 6.01 (s, 1H), 4.35 (d, J=17.2 Hz, 1H), 4.07-4.00 (m, 3H), 3.84-3.82 (m, 1H), 3.68-3.65 (m, 1H), 3.33-3.17 (m, 2H), 3.03-2.91 (m, 4H), 2.62-2.58 (m, 1H), 2.53 (d, J=2.0 Hz, 3H), 2.01-1.98 (m, 2H), 1.21 (d, J=6.8 Hz, 3H), 1.10 (t, J=7.2 Hz, 3H).

Compound 37F: LC-MS (ESI): mass calcd. for C₂₈H₃₂F₃N₅O₄S 591.2, m/z found 592.3 [M+H]⁺. ¹H NMR (400 MHz, CDCl₃) δ 9.26 (s, 1H), 7.83 (d, J=2.8 Hz, 1H), 7.41 (d, J=2.8 Hz, 1H), 7.09-7.03 (m, 1H), 6.99-6.96 (m, 1H), 6.92-6.88 (m, 1H), 6.01 (s, 1H), 4.32 (d, J=17.2 Hz, 1H), 4.10-3.99 (m, 3H), 3.90-3.85 (m, 1H), 3.58-3.52 (m, 1H), 3.46-3.34 (m, 2H), 3.08-2.97 (m, 2H), 2.79-2.74 (m, 1H), 2.68-2.59 (m, 2H), 2.54 (s, 3H), 2.18-2.10 (m, 1H), 2.06-2.00 (m, 1H), 1.24 (d, J=7.2 Hz, 3H), 1.09 (t, J=7.2 Hz, 3H).

### Compound 38A: 1-((4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)methyl)cyclopropane-1-carboxylic acid

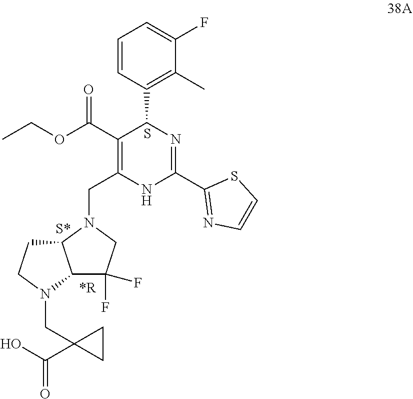

### Preparation of Intermediate S13

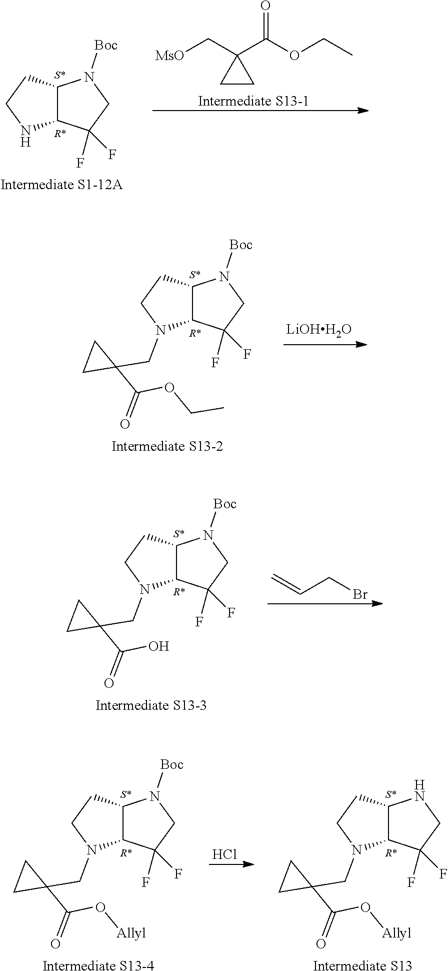

### S13-1: Ethyl 1-(((methylsulfonyl)oxy)methyl)cyclopropanecarboxylate

To a solution of ethyl 1-(hydroxymethyl)cyclopropanecarboxylate (1.25 g, 95% purity, 8.70 mmol) in dichloromethane (15 mL) was added triethylamine (1.05 g, 10.4 mmol) at 0° C. under nitrogen atmosphere. Then methanesulfonyl chloride (1.09 g, 9.50 mmol) was added. After stirred at room temperature under nitrogen atmosphere for 1 hour, the mixture was partitioned between water (15 mL) and dichloromethane (15 mL). The organic layer was washed with water (20 mL), dried over Na₂SO₄₍ₛ₎ and filtered. The filtrate was concentrated to give the title compound (1.87 g, 90% purity from ¹H NMR, 97% yield) as yellow oil. ¹H NMR (400 MHz, CDCl₃) δ 4.34 (s, 2H), 4.16 (dd, J=7.2, 14.4 Hz, 2H), 3.08 (s, 3H), 1.44-1.42 (m, 2H), 1.26 (t, J=7.2 Hz, 3H), 1.06-1.03 (m, 2H).

### S13-2: (cis)-tert-Butyl 4-((1-(ethoxycarbonyl)cyclopropyl)methyl)-3,3-difluorohexahydropyrrolo[3,2-b]pyrrole-1(2H)-carboxylate

To a solution of S1-12A (150 mg, 95% purity, 0.574 mmol) and S13-1 (200 mg, 90% purity, 0.810 mmol) in acetonitrile (4 mL) was added potassium carbonate (158 mg, 1.14 mmol). After stirred at 80° C. overnight, the mixture was filtered. The filtrate was concentrated to give a residue, which was purified by C18 column (acetonitrile:water=30% to 90%) to afford the desired product (120 mg, 82% purity, 46% yield) as colorless oil. LC-MS (ESI): mass calcd. for C₁₈H₂₈F₂N₂O₄ 374.2, m/z found 375.4 [M+H]⁺.

### S13-3: 1-(((cis)-4-(tert-Butoxycarbonyl)-6,6-difluorohexahydropyrrolo[3,2-b]pyrrol-1(2H)-yl)methyl)cyclopropanecarboxylic acid

To a solution of S13-2 (100 mg, 82% purity, 0.219 mmol) in methanol (3 mL) and water (1 mL) was added lithium hydroxide hydrate (50 mg, 1.19 mmol). The mixture was stirred at room temperature for 24 hours. Then it was acidified with 2 M hydrochloride to pH=3 and extracted with ethyl acetate (20 mL) for three times. The combined organic phases were washed with brine (20 mL), dried over Na₂SO₄₍ₛ₎, filtered and concentrated to afford the desired product (80 mg, 90% purity, 95% yield) as colorless oil. LC-MS (ESI): mass calcd. for C₁₆H₂₄F₂N₂O₄ 346.2, m/z found 347.5 [M+H]⁺.

### S13-4: (cis)-tert-Butyl 4-((1-((allyloxy)carbonyl)cyclopropyl)methyl)-3,3-difluorohexahydropyrrolo[3,2-b]pyrrole-1(2H)-carboxylate

To a solution of S13-3 (100 mg, 90% purity, 0.26 mmol) in N,N-dimethylformamide (3 mL) was added potassium carbonate (120 mg, 0.868 mmol) and allyl bromide (78 mg, 0.645 mmol). After stirred at room temperature overnight under nitrogen, the mixture was added ethyl acetate (50 mL). The organic solution was washed with water (10 mL) twice and brine (10 mL), dried over Na₂SO₄₍ₛ₎, filtered and concentrated. The residue was purified by C18 column (acetonitrile:water=20% to 80%) to afford the desired product (90 mg, 90% yield) as white solids. LC-MS (ESI): mass calcd. for C₁₉H₂₈F₂N₂O₄ 386.2, m/z found 387.5 [M+H]⁺.

### S13: Allyl 1-(((cis)-6,6-difluorohexahydropyrrolo[3,2-b]pyrrol-1(2H)-yl)methyl)cyclopropanecarboxylate

To a solution of S13-4 (90 mg, 0.233 mmol) in ethyl acetate (1 mL) was added 4 M hydrochloride in ethyl acetate (1 mL, 4 mmol). After stirred at room temperature for 1 hour, the mixture was concentrated to give a residue, which was diluted with ethyl acetate (50 mL). The organic layer was washed with saturated sodium carbonate solution (10 mL) for three times, brine (10 mL), dried over Na₂SO₄₍ₛ₎, filtered and concentrated to afford the desired product (70 mg, 90% purity, 94% yield) as colorless oil. LC-MS (ESI): mass calcd. for C₁₉H₂₈F₂N₂O₄ 286.2, m/z found 287.5 [M+H]⁺.

### Compound 38A: 1-((4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)methyl)cyclopropane-1-carboxylic acid

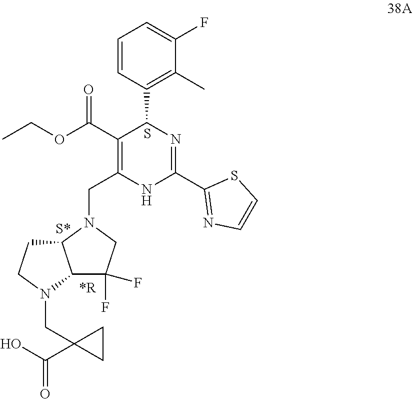

Compound 38A was H2-1A with S13 according to Typical coupling method 1 and 2 successively. Purified by C18 column (acetonitrile:water=20% to 80%) to afford the desired product (61 mg, 96% purity, 62% yield) as yellow solids. LC-MS (ESI): mass calcd. for C₂₉H₃₂F₃N₅O₄S 603.2, m/z found 604.3 [M+H]⁺. ¹H NMR (400 MHZ, CDCl₃) δ 9.27 (s, 1H), 8.86 (d, J=2.8 Hz, 1H), 7.41 (d, J=2.8 Hz, 1H), 7.10-7.04 (m, 1H), 6.99-6.97 (m, 1H), 6.93-6.89 (m, 1H), 6.01 (s, 1H), 4.32-4.28 (m, 1H), 4.11-3.87 (m, 3H), 3.89-3.87 (m, 1H), 3.56-3.47 (m, 3H), 3.38-3.36 (m, 1H), 3.05-3.02 (m, 1H), 2.71-2.65 (m, 1H), 2.53 (d, J=2 Hz, 3H), 2.38-2.35 (m, 1H), 2.12-2.03 (m, 2H), 1.51-1.46 (m, 1H), 1.37-1.33 (m, 1H), 1.11 (t, J=6.8 Hz, 3H), 0.76-0.65 (m, 2H).

Compound 39A: 3-(4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-1)cyclobutane-1-carboxylic acid

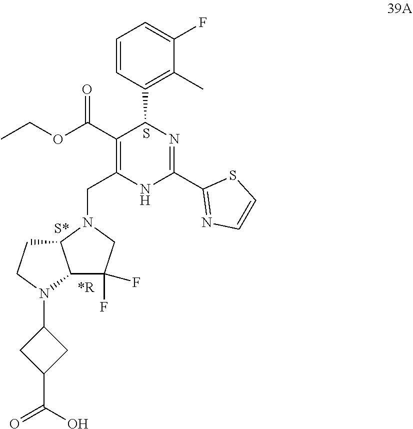

### Preparation of Intermediate S14

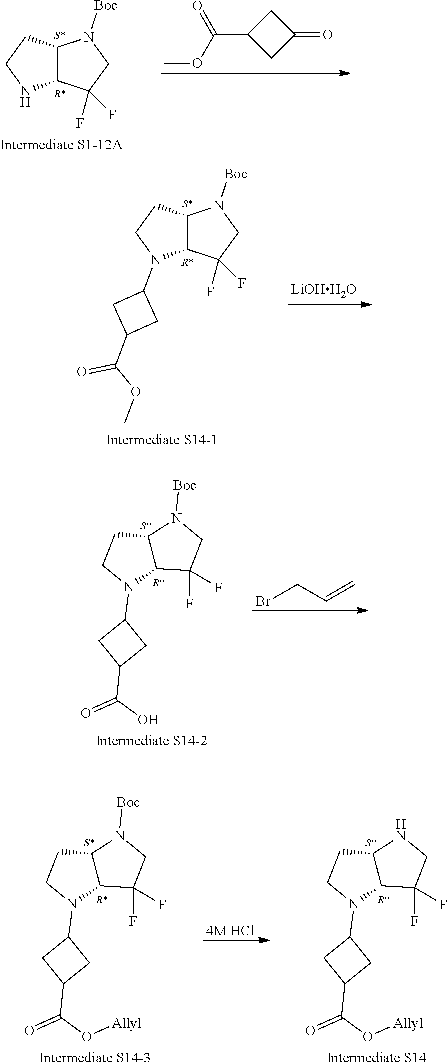

### S14-1: (cis)-tert-Butyl 3,3-difluoro-4-(3-(methoxycarbonyl)cyclobutyl)hexahydropyrrolo[3,2-b]pyrrole-1(2H)-carboxylate

To a solution of S1-12A (300 mg, 95% purity, 1.15 mmol) in 1,2-dichloroethane (20 mL) was added methyl 3-oxocyclobutanecarboxylate (180 mg, 1.41 mmol) and acetic acid (185 mg, 3.08 mmol) at room temperature under nitrogen atmosphere. The mixture was stirred at room temperature for 1 hour. Then sodium triacetoxyhydroborate (730 mg, 3.44 mmol) was added into the mixture at 0° C. After stirred at room temperature overnight, the mixture was poured into water (30 mL) and extracted with ethyl acetate (30 mL) for three times. The combined organic layers were washed with brine (50 mL) and dried over Na₂SO₄₍ₛ₎, filtered and concentrated to give a crude, which was purified by C18 column (acetonitrile:water=60% to 80%) to give the desired compound (346 mg, 84% yield) as colorless oil. LC-MS (ESI): mass calcd. for C₁₇H₂₆F₂N₂O₄ 360.4, m/z found 361.4 [M+H]⁺.

### S14-2: 3-((cis)-4-(tert-Butoxycarbonyl)-6,6-difluorohexahydropyrrolo[3,2-b]pyrrol-1(2H)-yl)cyclobutanecarboxylic acid

To a solution of S14-1 (346 mg, 0.960 mmol) in methanol (6 mL) was added a solution of lithium hydroxide monohydrate (80 mg, 1.91 mmol) in water (3 mL). After stirred at 30° C. for 2 hours, the mixture was concentrated to give the title compound (288 mg, 71% purity, 61% yield) as pale yellow solids. LC-MS (ESI): mass calcd. for C₁₆H₂₄F₂N₂O₄ 346.4, m/z found 347.4 [M+H]⁺.

### S14-3: (cis)-tert-Butyl 4-(3-((allyloxy)carbonyl)cyclobutyl)-3,3-difluorohexahydropyrrolo[3,2-b]pyrrole-1(2H)-carboxylate

To a mixture of S14-2 (288 mg, 71% purity, 0.590 mmol) and potassium carbonate (249 mg, 1.80 mmol) in N,N-dimethylformamide (5 mL) was added 3-bromoprop-1-ene (178 mg, 1.47 mmol). After stirred at room temperature for 3 hours under nitrogen, the mixture was poured into water (30 mL) and extracted with ethyl acetate (30 mL) for three times. The combined organic layers were washed with brine (50 mL) and dried over Na₂SO₄₍ₛ₎, filtered and concentrated to give a crude, which was purified by silica gel column chromatography (petroleum ether:ethyl acetate=5:1) to give the desired product (264 mg, 85% purity by ¹H NMR, 98% yield) as yellow oil. ¹H NMR (400 MHz, CDCl₃) δ 5.97-5.87 (m, 1H), 5.35-5.22 (m, 2H), 4.61-4.57 (m, 2H), 4.54-4.39 (m, 1H), 3.91-3.75 (m, 1H), 3.64-3.55 (m, 1H), 3.42-3.21 (m, 2H), 3.08-2.98 (m, 1H), 2.78-2.59 (m, 2H), 2.41-2.14 (m, 4H), 2.02-1.93 (m, 1H), 1.86-1.80 (m, 1H), 1.46 (m, 9H).

### S14: Allyl 3-((cis)-6,6-difluorohexahydropyrrolo[3,2-b]pyrrol-1(2H)-yl)cyclobutanecarboxylate

To a solution of S14-3 (262 mg, 85% purity, 0.576 mmol) in ethyl acetate (5 mL) was added 4 M hydrochloride in ethyl acetate (1 mL, 4.0 mmol). After stirred at room temperature for 2 hours, the mixture was concentrated to give a crude, which was washed with saturated sodium bicarbonate aqueous solution and extracted with ethyl acetate (30 mL) for three times. The combined organic layers were washed with brine (30 mL), dried over Na₂SO₄₍ₛ₎, filtered and concentrated to give a crude (190 mg, 72% purity, 83% yield) as pale yellow solids. LC-MS (ESI): mass calcd. for C₁₄H₂₀F₂N₂O₄ 386.4, m/z found 387.4 [M+H]⁺.

### Compound 39A: 3-(4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)cyclobutane-1-carboxylic acid

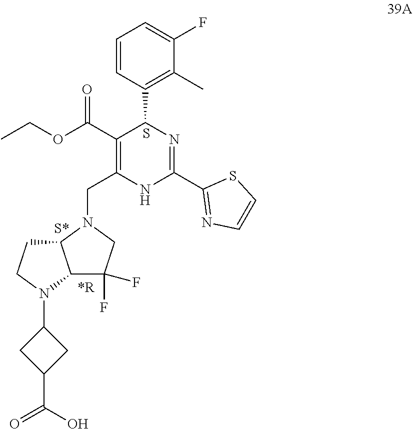

Compound 39A was from H2-1A and S14 according typical method 1 and 2 successively. Purified by C18 column (acetonitrile:water=40% to 75%) to give the desired product (18.3 mg, 99% purity, 43% yield) as yellow solids. LC-MS (ESI): mass calcd. for C₂₉H₃₂F₃N₅O₄S 603.7, m/z found 604.2 [M+H]⁺. ¹H NMR (400 MHz, CDCl₃) δ 9.30 (br s, 1H), 7.84-7.82 (m, 1H), 7.40 (d, J=2.8 Hz, 1H), 7.10-7.04 (m, 1H), 7.10-6.98 (m, 1H), 6.92-6.89 (m, 1H), 6.01 (s, 1H), 4.29-4.23 (m, 1H), 4.10-3.97 (m, 3H), 3.87-3.77 (m, 1H), 3.56-3.41 (m, 2H), 3.31-3.15 (m, 2H), 2.98-2.86 (m, 2H) 2.53 (s, 3H), 2.32-2.21 (m, 5H), 1.98-1.86 (m, 2H), 1.11 (t, J=7.2 Hz, 3H).

### Compound 40: (cis)-3-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)cyclobutane-1-carboxylic acid (Single Diastereomer)

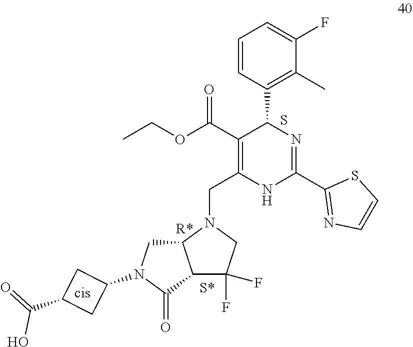

This compound was made using the procedure similar to Compound 42 by replacing tert-butyl 2,2-dimethyl-3-oxopropanoate with tert-butyl 3-oxocyclobutane-1-carboxylate and replacing H5-1A with H2-1A. Purified by C18 column (acetonitrile:water=60% to 80%) to give the desired compound (27 mg, 98.3% purity, 46% yield) as yellow solids. LC-MS (ESI): mass calcd. for C₂₉H₃₀F₃N₅O₅S 617.2, m/z found 618.2. ¹H NMR (400 MHz, CD₃OD) δ 7.90 (d, J=3.2 Hz, 1H), 7.72 (d, J=3.2 Hz, 1H), 7.22-7.14 (m, 2H), 6.97-6.91 (m, 1H), 5.99 (m, 1H), 4.60-4.54 (m, 1H), 4.41-4.35 (m, 1H), 4.20 (d, J=16.4 Hz, 1H), 4.07 (q, J=7.2 Hz, 2H), 3.91-3.86 (m, 1H), 3.74-3.63 (m, 2H), 3.59-3.49 (m, 1H), 3.43-3.40 (m, 1H), 3.11-2.99 (m, 1H), 2.89-2.79 (m, 1H), 2.52 (s, 3H), 2.49-2.39 (m, 3H), 2.34-2.26 (m, 1H), 1.14 (t, J=7.2 Hz, 3H).

### Compound 41: 3-((cis)-1-((6-(2-chloro-4-fluorophenyl)-5-(methoxycarbonyl)-2-(5-methyloxazol-4-yl)-3,6-dihydropyrimidin-4-yl)methyl)-3,3-difluoro-4-oxohexahydropyrrolo[3,4-b]pyrrol-5(1H)-yl)-2,2-dimethylpropanoic acid (Single Diastereomer)

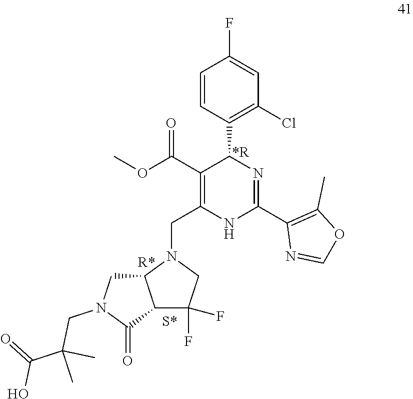

This compound was made using the procedure similar to Compound 42 by replacing H5-1A with H18-1A. Purified by C18 column (acetonitrile:water=5% to 95%) to give the title compound (31.6 mg, 99.9% purity) as yellow solids. LC-MS (ESI): mass calcd. for C₂₈H₂₉ClF₃N₅O₆ 623.2, m/z found 624.3 [M+H]⁺. ¹H NMR (400 MHz, CD₃OD) δ 7.86 (s, 1H), 7.29-7.25 (m, 1H), 7.12 (dd, J=8.4, 2.4 Hz, 1H), 6.94 (td, J=8.4, 2.4 Hz, 1H), 6.02 (s, 1H), 4.22 (d, J=16.4 Hz, 1H), 3.98 (d, J=16.4 Hz, 1H), 3.72-3.69 (m, 1H), 3.54-3.53 (m, 1H), 3.50 (s, 3H), 3.49-3.36 (m, 3H), 3.29-3.27 (m, 2H), 2.91-2.81 (m, 1H), 2.41 (s, 3H), 1.08 (s, 3H), 1.07 (s, 3H).

### Preparation of Intermediate T11

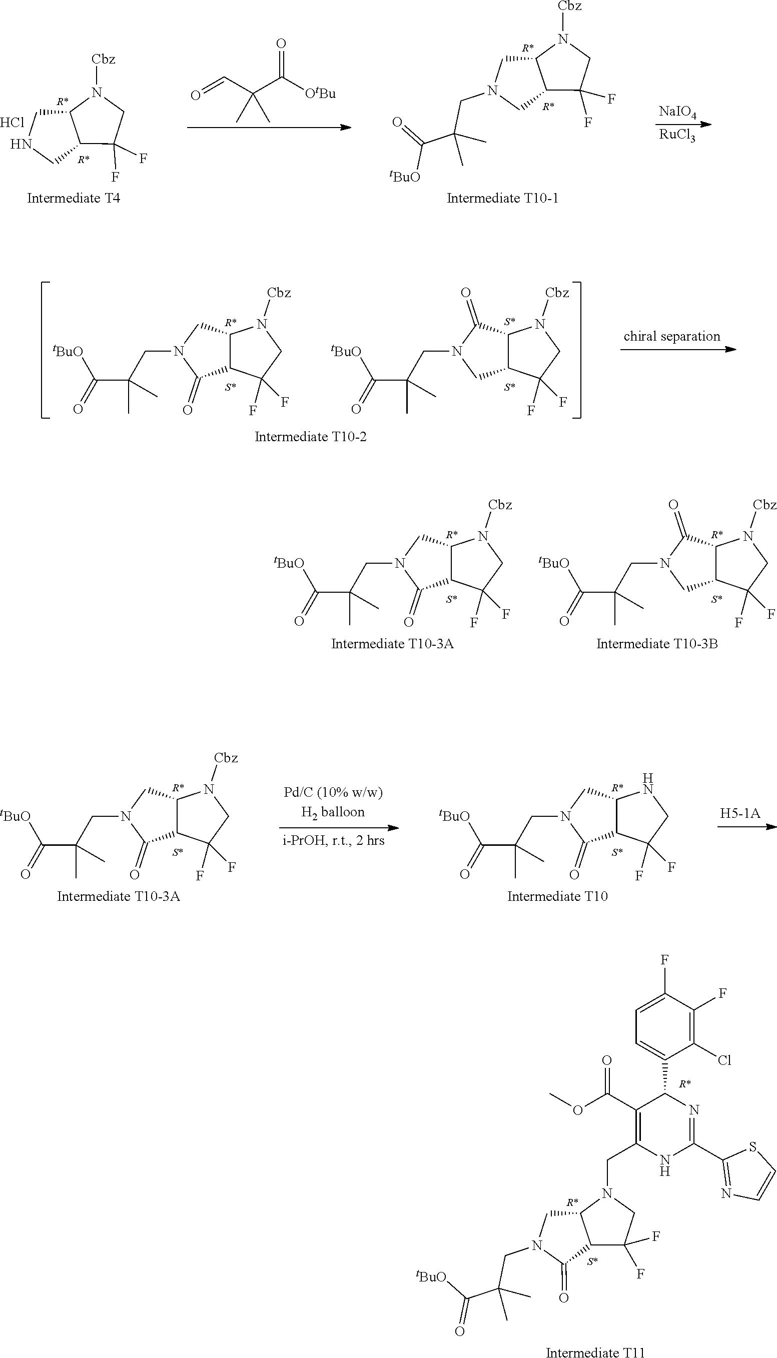

### T10-1: (cis)-Benzyl 5-(3-(tert-butoxy)-2,2-dimethyl-3-oxopropyl)-3,3-difluorohexahydropyrrolo[3,4-b]pyrrole-1(2H)-carboxylate

This compound was made from T4 and tert-butyl 2,2-dimethyl-3-oxopropanoate according to typical method 5. LC-MS (ESI): mass calcd. for C₂₃H₃₂F₂N₂O₄ 438.5, m/z found 439.2 [M+H]⁺. ¹H NMR (400 MHz, CDCl₃) δ 7.39-7.30 (m, 5H), 5.16-5.08 (m, 2H), 4.51-4.43 (m, 1H), 4.11-3.95 (m, 1H), 3.63-3.53 (m, 1H), 3.22-3.15 (m, 1.5H), 3.01-2.85 (m, 1.5H), 2.59-2.41 (m, 3H), 2.33-2.27 (m, 1H), 1.40-1.39 (m, 9H), 1.10-1.08 (m, 6H).

### T10-2: Mixture of (cis)-benzyl 5-(3-(tert-butoxy)-2,2-dimethyl-3-oxopropyl)-3,3-difluoro-4-oxohexahydropyrrolo[3,4-b]pyrrole-1(2H)-carboxylate and (cis)-benzyl 5-(3-(tert-butoxy)-2,2-dimethyl-3-oxopropyl)-3,3-difluoro-6-oxohexahydropyrrolo[3,4-b]pyrrole-1(2H)-carboxylate

To a mixture of T10-1 (1.40 g, 90% purity, 2.87 mmol) in ethyl acetate (15 mL) and water (15 mL) was added sodium periodate (1.30 g, 6.08 mmol) and ruthenium(III) chloride (180 mg, 0.868 mmol) at room temperature. After stirred at room temperature for 30 minutes, the mixture was quenched by saturated aqueous sodium sulfite (15 mL), extracted with ethyl acetate (50 mL) twice. The combined organic layers were washed with brine (30 mL), dried over NasSO₄₍ₛ₎, filtered and concentrated to give a residue, which was purified by silica gel column chromatography (petroleum ether:ethyl acetate=5:1) to give the title compound (1.00 g, 90% purity from ¹H NMR, 69% yield) as light yellow oil. ¹H NMR (400 MHz, CDCl₃) δ 7.48-7.34 (m, 5H), 5.29-5.09 (m, 2H), 4.66-4.58 (m, 1H), 4.10-3.96 (m, 1H), 3.76-3.57 (m, 2H), 3.51-3.09 (m, 4H), 1.44-1.37 (m, 9H), 1.15-1.06 (m, 6H).

### T10-3A and T10-3B: (cis)-Benzyl 5-(3-(tert-butoxy)-2,2-dimethyl-3-oxopropyl)-3,3-difluoro-4-oxohexahydropyrrolo[3,4-b]pyrrole-1(2H)-carboxylate and (cis)-benzyl 5-(3-(tert-butoxy)-2,2-dimethyl-3-oxopropyl)-3,3-difluoro-6-oxohexahydropyrrolo[3,4-b]pyrrole-1(2H)-carboxylate

T10-2 (1.00 g, 90% purity, 1.99 mmol) was separated by chiral Prep. HPLC (Column: Chiralpak IG 5 μm 20*250 mm; Mobile Phase: Hex:EtOH=40:60 at 15 mL/min; Temp: 30° C.; Wavelength: 214 nm) to give T10-3A (330 mg, 90% purity from ¹H NMR, 33% yield) and T10-3B (380 mg, 90% purity from ¹H NMR, 38% yield) as light brown oil.

T10-3A: ¹H NMR (400 MHz, CDCl₃) δ 7.42-7.32 (m, 5H), 5.21-5.09 (m, 2H), 4.66-4.58 (m, 1H), 4.07-3.93 (m, 1H), 3.77-3.58 (m, 3H), 3.42-3.33 (m, 2H), 3.06-2.98 (m, 1H), 1.44 (s, 5H), 1.37 (s, 4H), 1.11 (s, 6H).

T10-3B: ¹H NMR (400 MHz, CDCl₃) δ 7.48-7.31 (m, 5H), 5.29-5.15 (m, 2H), 5.05-4.84 (m, 1H), 4.22-4.08 (m, 1H), 3.66-3.54 (m, 1H), 3.47-3.42 (m, 3H), 3.23-3.11 (m, 1H), 3.06-3.00 (m, 1H), 1.46 (s, 9H), 1.15-1.14 (m, 6H).

### T10: tert-Butyl 3-((cis)-3,3-difluoro-4-oxohexahydropyrrolo[3,4-b]pyrrol-5(1H)-yl)-2,2-dimethylpropanoate

To a solution of T10-3A (300 mg, 90% purity, 0.597 mmol) in isopropyl alcohol (8 mL) was added 10% palladium on charcoal wt. (30 mg), then the reaction was stirred at room temperature under hydrogen atmosphere of balloon for 2 hours. The mixture was filtered and concentrated to afford the desired product (205 mg, 90% purity from ¹H NMR, 97% yield) as colorless oil. ¹H NMR (400 MHz, CDCl₃) δ 4.16-4.10 (m, 1H), 3.64-3.60 (m, 2H), 3.33-3.30 (m, 2H), 3.25-3.13 (m, 2H), 3.01 (br s, 1H), 1.46 (s, 9H), 1.16 (s, 3H), 1.15 (s, 3H).

### T11: Methyl 6-(((cis)-5-(3-(tert-butoxy)-2,2-dimethyl-3-oxopropyl)-3,3-difluoro-4-oxohexahydropyrrolo[3,4-b]pyrrol-1(2H)-yl)methyl)-4-(2-chloro-3,4-difluorophenyl)-2-(thiazol-2-yl)-1,4-dihydropyrimidine-5-carboxylate

This compound was made from H5-1A and T10 according to typical method 1. LC-MS (ESI): mass calcd. for C₃₁H₃₄ClF₄N₅O₅S 699.2, m/z found 700.4 [M+H]⁺. ¹H NMR (400 MHz, CDCl₃) δ 9.31 (s, 1H), 7.86 (d, J=2.8 Hz, 0.1H), 7.79 (d, J=3.2 Hz, 0.9H), 7.54 (d, J=3.2 Hz, 0.1H), 7.43 (d, J=3.2 Hz, 0.9H) 7.10-6.98 (m, 2H), 6.20 (s, 0.9H), 6.08 (s, 0.1H), 4.42 (d, J=17.2 Hz, 1H), 3.92 (d, J=16.8 Hz, 1H), 3.76-3.72 (m, 1H), 3.67 (d, J=14.0 Hz, 1H), 3.59 (s, 3H), 3.53-3.30 (m, 5H), 2.89-2.80 (m, 1H), 1.34 (s, 9H), 1.24 (s, 3H), 1.19 (s, 3H).

### Compound 42: 3-((cis)-1-((6-(2-Chloro-3,4-difluorophenyl)-5-(methoxycarbonyl)-2-(thiazol-2-yl)-3,6-dihydropyrimidin-4-yl)methyl)-3,3-difluoro-4-oxohexahydropyrrolo[3,4-b]pyrrol-5(1H)-yl)-2,2-dimethylpropanoic acid (Single Diastereomer)

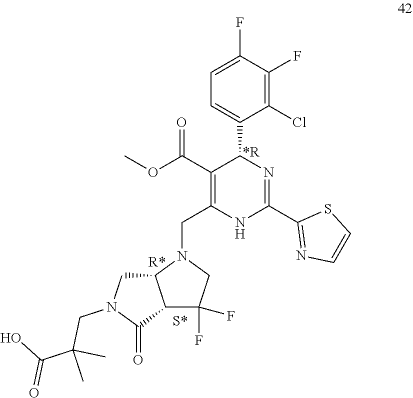

This compound was made from T11 according to typical method 1. LC-MS (ESI): mass calcd. for C₂₇H₂₆ClF₄N₅O₅S 643.1, m/z found 644.2 [M+H]⁺. ¹H NMR (400 MHz, CDCl₃) δ 7.89 (d, J=3.2 Hz, 1H), 7.74 (d, J=3.2 Hz, 1H), 7.31-7.23 (m, 2H), 6.19 (s, 1H), 4.46-4.35 (m, 1H), 4.10 (d, J=16.8 Hz, 1H), 3.86-3.82 (m, 1H), 3.67-3.50 (m, 7H), 3.43-3.35 (m, 2H), 3.06-2.91 (m, 1H), 1.21 (s, 3H), 1.20 (s, 3H).

### Compound 43: 3-((cis)-1-((6-(2-chloro-3-fluorophenyl)-5-(ethoxycarbonyl)-2-(thiazol-2-yl)-3,6-dihydropyrimidin-4-yl)methyl)-3,3-difluoro-4-oxohexahydropyrrolo[3,4-b]pyrrol-5(1H)-yl)-2,2-dimethylpropanoic acid (Single Diastereomer)

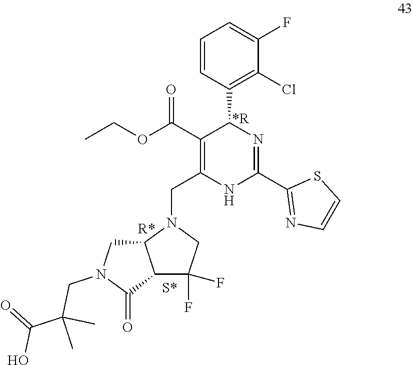

This compound was made using the procedure similar to Compound 42 by replacing H5-1A with H1-1A. Purified by C18 column (acetonitrile:water=5% to 100%) to give the title compound (40 mg, 67% yield, 99.6% purity) as yellow oil. LC-MS (ESI): mass calcd. for C₂₈H₂₉ClF₃N₅O₅S 639.2, m/z found 640.1 [M+H]⁺. ¹H NMR (400 MHz, CDCl₃) δ 9.22 (br s, 1H), 7.83 (d, J=3.2 Hz, 1H), 7.46 (d, J=3.6 Hz, 1H), 7.21-7.18 (m, 2H), 7.07-7.03 (m, 1H), 6.28 (s, 1H), 4.55 (d, J=15.6 Hz, 1H), 4.10-3.99 (m, 3H), 3.87-3.78 (m, 2H), 3.62-3.58 (m, 1H), 3.44-3.26 (m, 3H), 2.91 (d, J=14 Hz, 1H), 2.84-2.74 (m, 1H), 1.39 (s, 3H), 1.36 (s, 3H), 1.11 (t, J=6.8 Hz, 3H).

### Compound 44A and 44B: 4-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)cyclohexane-1-carboxylic acid (Single Diastereomer)

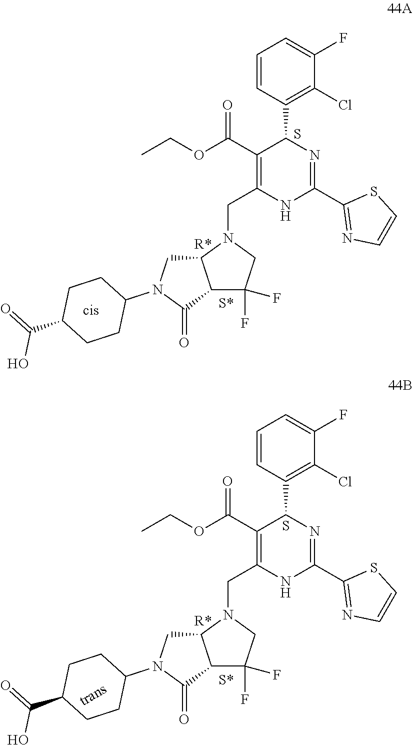

This compound was made using the procedure similar to Compound 42 by replacing tert-butyl 2,2-dimethyl-3-oxopropanoate with tert-butyl 4-oxocyclohexane-1-carboxylate and replacing H5-1A with H2-1A.

44A, LC-MS (ESI): mass calcd. for C₃₁H₃₄F₃N₅O₅S 645.7, m/z found 646.3 [M+H]⁺. ¹H NMR (400 MHz, CDCl₃) δ 9.06 (s, 1H), 7.77 (d, J=3.2 Hz, 1H), 7.39 (d, J=3.2 Hz, 1H), 7.13-7.07 (m, 1H), 7.00-6.98 (m, 1H), 6.93-6.88 (m, 1H), 6.01 (s, 1H), 4.39 (d, J=17.2 Hz, 1H), 4.09-4.00 (m, 4H), 3.81-3.75 (m, 1H), 3.48-3.34 (m, 4H), 2.97-2.87 (m, 1H), 2.68 (s, 1H), 2.52 (d, J=2.0 Hz, 3H), 2.28-2.23 (m, 1H), 2.16-2.09 (m, 1H), 1.69-1.60 (m, 6H), 1.09 (t, J=6.8 Hz, 3H).

44B, LC-MS (ESI): mass calcd. for C₃₁H₃₄F₃N₅O₅S 645.7, m/z found 646.3 [M+H]⁺. ¹H NMR (400 MHz, CDCl₃) δ 9.07 (s, 1H), 7.75 (d, J=2.8 Hz, 1H), 7.40 (d, J=3.2 Hz, 1H), 7.10-7.04 (m, 1H), 6.98-6.87 (m, 2H), 6.02 (s, 1H), 4.43 (d, J=16.8 Hz, 1H), 4.06-3.98 (m, 4H), 3.82-3.75 (m, 1H), 3.50-3.30 (m, 4H), 2.96-2.87 (m, 1H), 2.53 (s, 3H), 2.19-2.12 (m, 3H), 1.97-1.89 (m, 2H), 1.61-1.51 (m, 2H), 1.46-1.38 (m, 2H), 1.10 (t, J=6.8 Hz, 3H).

### Compound 45: 3-((cis)-1-((5-(ethoxycarbonyl)-6-(3-fluoro-2-methylphenyl)-2-(5-methyloxazol-4-yl)-3,6-dihydropyrimidin-4-yl)methyl)-3,3-difluoro-4-oxohexahydropyrrolo[3,4-b]pyrrol-5(1H)-yl)-2,2-dimethylpropanoic acid (Single Diastereomer)

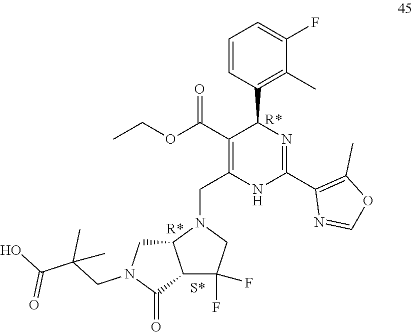

This compound was made using the procedure similar to Compound 42 by replacing H5-1A with H15-1A. Purified by Prep. HPLC (Column: Waters Xbrige C18 (5 μm 19*150 mm), Mobile phase A: water (0.1% ammonium bicarbonate), Mobile phase B: acetonitrile, UV: 214 nm, Flow rate: 15 mL/min, Gradient: 26-65% (% B)) to give the title compound (65 mg, 98.8% purity) as yellow solids. LC-MS (ESI): mass calcd. for C₃₀H₃₄F₃N₅O₆S 617.6, m/z found 618.2 [M+H]⁺. ¹H NMR (400 MHz, CDCl₃) δ 8.83 (br s, 1H), 7.79 (s, 1H), 7.11-7.06 (m, 1H), 7.00-6.98 (m, 1H), 6.90 (t, J=8.8 Hz, 1H), 5.99 (s, 1H), 4.56-4.53 (m, 1H), 4.05-4.01 (m, 3H), 3.86-3.79 (m, 2H), 3.62 (t, J=6.8 Hz, 1H), 3.44-3.39 (m, 1H), 3.37-3.30 (m, 1H), 3.28-3.25 (m, 1H), 2.94 (d, J=14.0 Hz, 1H), 2.85-2.75 (m, 1H), 2.55 (s, 3H), 2.52 (s, 3H), 1.33 (s, 6H), 1.10 (t, J=6.0 Hz, 3H).

### Compound 46: 3-(((cis)-1-(((S)-5-(Ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)sulfonyl)-2,2-dimethylpropanoic acid (Single Diastereomer)

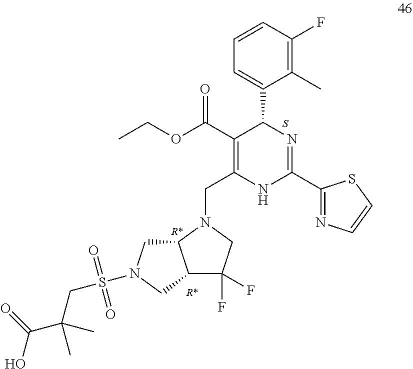

To a solution of T9 (60 mg, 90% purity, 0.074 mmol) in dichloromethane (1 mL) was added trifluoroacetic acid (1 mL) at 0° C. After stirred at room temperature for 1 hours, the mixture was concentrated under reduced pressure to give crude, which was purified by C18 column (acetonitrile:water (add 0.1% ammonium bicarbonate)=30% to 40%) to give the title compound (19.0 mg, 96.1% purity, 37% yield) as yellow solids. LC-MS (ESI): mass calcd. for C₂₉H₃₄F₃N₅O₆S₂ 669.7, m/z found 670.3 [M+H]⁺. ¹H NMR (400 MHz, CD₃OD) δ 7.96 (d, J=3.6 Hz, 1H), 7.74 (d, J=3.6 Hz, 1H), 7.18-7.15 (m, 2H), 6.97-6.92 (m, 1H), 5.99 (s, 1H), 4.37-4.29 (m, 1H), 4.22-4.18 (m, 1H), 4.08 (q, J=7.2 Hz, 2H), 3.91-3.85 (m, 2H), 3.76-3.71 (m, 1H), 3.59-3.41 (m, 5H), 3.29-3.24 (m, 1H), 3.13-3.03 (m, 1H), 2.52 (s, 3H), 1.24 (s, 3H), 1.17-1.13 (m, 6H).

### Compound 47: 3-((cis)-1-((5-(ethoxycarbonyl)-6-(3-fluoro-2-methylphenyl)-2-(1-methyl-1H-imidazol-2-yl)-3,6-dihydropyrimidin-4-yl)methyl)-3,3-difluoro-4-oxohexahydropyrrolo[3,4-b]pyrrol-5(1H)-yl)-2,2-dimethylpropanoic acid (Single Diastereomer)

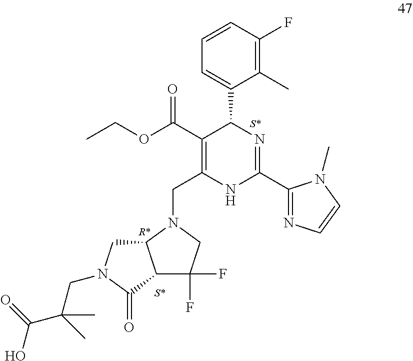

This compound was made using the procedure similar to Compound 42 by replacing H5-1A with H28-1B. Purified by C18 column (acetonitrile:water=30% to 90%) to give the desired compound (22 mg, 95.7% purity, 85% yield) as yellow solids. LC-MS (ESI): mass calcd. for C₃₀H₃₅F₃N₆O₅ 616.3, m/z found 617.3 [M+H]⁺. ¹H NMR (400 MHz, CD₃OD) δ 7.20-7.15 (m, 2H), 7.11-7.09 (m, 1H), 6.99-6.97 (m, 1H), 6.95-6.93 (m, 1H), 6.03 (s, 1H), 4.33 (d, J=15.6 Hz, 1H), 4.11-4.09 (m, 1H), 4.08-4.04 (m, 2H), 3.88 (s, 3H), 3.84-3.81 (m, 1H), 3.71-3.66 (m, 2H), 3.62-3.48 (m, 2H), 3.43-3.37 (m, 2H), 3.02-2.92 (m, 1H), 2.52 (s, 3H), 1.21 (s, 3H), 1.20 (s, 3H), 1.12 (t, J=7.2 Hz, 3H).

### Preparation of Intermediate T8

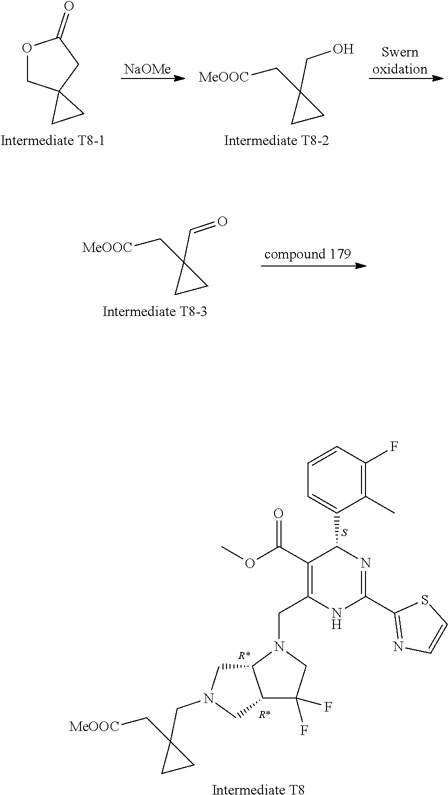

### T8-2: Methyl 2-(1-(hydroxymethyl)cyclopropyl)acetate

To a solution of 5-oxaspiro[2.4]heptan-6-one T8-1 (500 mg, 4.46 mmol) in methanol (10 mL) was added sodium methanolate (289 mg, 5.35 mmol) at 0° C. After 15 minutes, ammonium chloride (453 mg, 8.47 mmol) was added and the reaction mixture was stirred at 25° C. for 30 minutes. The reaction mixture was concentrated to give a residue, which was purified by silica gel column chromatography (petroleum ether:ethyl acetate=20:1 to 5:1) to give the title compound (600 mg, 90% purity from ¹H NMR, 84% yield) as colorless oil. ¹H NMR (400 MHz, CDCl₃) δ 3.70 (s, 3H), 3.49 (s, 2H), 2.60 (br s, 1H), 2.44 (s, 2H), 0.59-0.49 (m, 4H).

### T8-3: Methyl 2-(1-formylcyclopropyl)acetate

To a solution of oxalyl chloride (840 mg, 6.62 mmol) in dichloromethane (15 mL) was added dimethyl sulfoxide (1.10 g, 14.1 mmol) in dichloromethane (4 ml) at −78° C. under nitrogen atmosphere. After stirred at −78° C. for 20 minutes, methyl 2-(1-(hydroxymethyl)cyclopropyl)acetate T8-2 (530 mg, 90% purity, 3.31 mmol) in dichloromethane (6 mL) was added dropwise. The mixture was stirred at −78° C. for 20 minutes and triethylamine (2.70 g, 26.7 mmol) was added and warmed to room temperature, then stirred for another 0.5 hour. The reaction mixture was diluted with water (20 mL) and the organic layer was separated. The aqueous layer was extracted with dichloromethane (10 mL) for three times. The combined organic phases were washed with brine (10 mL), dried over Na₂SO₄₍ₛ₎ and filtered. The filtrate was concentrated to give title compound (520 mg, 90% purity from ¹H NMR, 99.5% yield) as colorless oil. ¹H NMR (400 MHz, CDCl₃) δ 8.71 (s, 1H), 3.69 (s, 3H), 2.60 (s, 2H), 1.34-1.30 (m, 2H), 1.11-1.08 (m, 2H).

### T8: (S)-Ethyl 6-(((cis)-3,3-difluoro-5-((1-(2-methoxy-2-oxoethyl)cyclopropyl)methyl)hexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate

The solution of Compound 179 (150 mg, 97% purity, 0.288 mmol), methyl 2-(1-formylcyclopropyl)acetate T8-3 (91 mg, 90% purity, 0.576 mmol) and acetic acid (36 mg, 0.599 mmol) in dichloromethane (3 mL) was stirred at 25° C. for 20 minutes. Then sodium triacetoxyborohydride (305 mg, 1.44 mmol) was added in portions and the obtained mixture was stirred at 25° C. for another 16 hours. The reaction mixture was adjusted to pH=8˜9 with saturated sodium bicarbonate aqueous solution (20 mL). The organic layer was separated, and the aqueous layer was extracted with dichloromethane (10 mL) for three times. The combined organic phases were washed with brine (10 mL), dried over Na₂SO₄₍ₛ₎ and filtered. The filtrate was concentrated to give a residue, which was purified by C-18 (acetonitrile:water=50% to 80%) to give the title compound (80 mg, 81% purity, 35.6% yield) as yellow solids. LC-MS (ESI): mass calcd. for C₃₁H₃₆F₃N₅O₄S 631.2, m/z found 632.5 [M+H]⁺.

### Compound 48: 2-(1-(((cis)-1-(((S)-5-(Ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)methyl)cyclopropyl)acetic acid (Single Diastereomer)

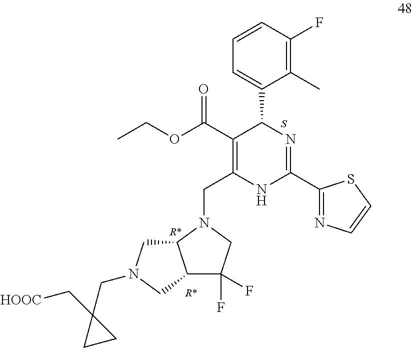

This compound was made from T8 according to typical method 4. LC-MS (ESI): mass calcd. for C₃₀H₃₄F₃N₅O₄S 617.2, m/z found 618.3 [M+H]⁺. ¹H NMR (400 MHz, CDCl₃) δ 9.38 (s, 0.9H), 7.82 (d, J=3.2 Hz, 0.1H), 7.80 (d, J=3.2 Hz, 0.9H), 7.54-7.52 (m, 0.1H), 7.40 (d, J=3.2 Hz, 0.9H), 7.22-7.14 (m, 0.1H), 7.11-7.06 (m, 1H), 7.02 (d, J=7.2 Hz, 1H), 6.93-6.88 (m, 1H), 6.02 (s, 0.9H), 5.96 (s, 0.1H), 4.40 (d, J=16.0 Hz, 1H), 4.09-3.98 (m, 3H), 3.67-3.64 (m, 1H), 3.40-3.30 (m, 3H), 3.07-2.78 (m, 4H), 2.64 (d, J=15.6 Hz, 1H), 2.54 (d, J=2.0 Hz, 3H), 2.51-2.42 (m, 1H), 2.17-2.09 (m, 2H), 1.11 (t, J=7.2 Hz, 3H), 0.74-0.70 (m, 1H), 0.54-0.37 (m, 3H).

### Compound 49: 2-(1-((4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)methyl)cyclopropyl)-acetic acid

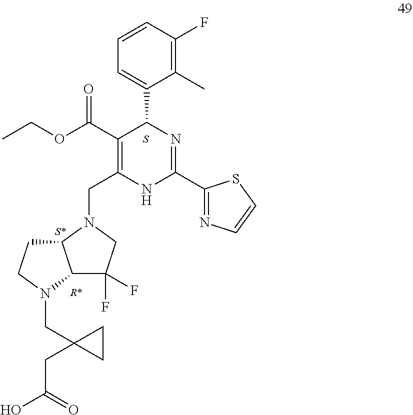

### Preparation of Intermediate 15

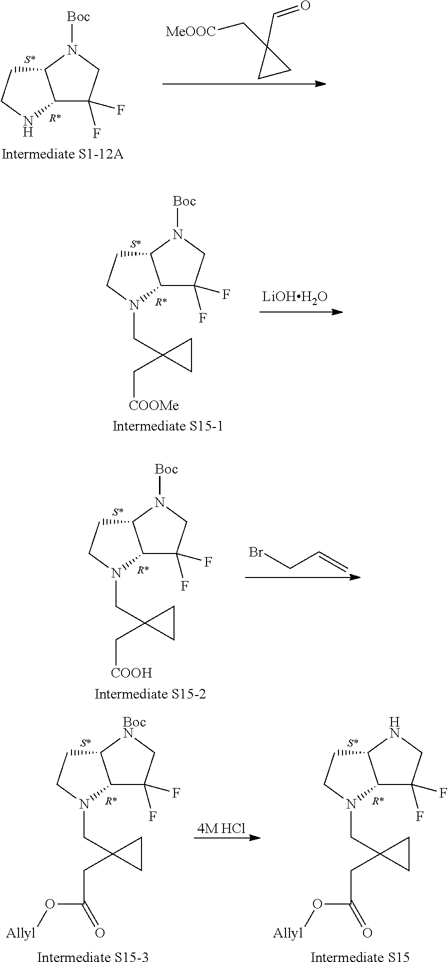

### S15-1: (cis)-tert-Butyl 3,3-difluoro-4-((1-(2-methoxy-2-oxoethyl)cyclopropyl)methyl)hexahydropyrrolo[3,2-b]pyrrole-1(2H)-carboxylate

The solution of S1-12A (100 mg, 90% purity, 0.363 mmol), methyl 2-(1-formylcyclopropyl)acetate (86 mg, 90% purity, 0.544 mmol) and acetic acid (46 mg, 0.766 mmol) in dichloromethane (5 mL) was stirred at 25° C. for 20 minutes. Then sodium triacetoxyborohydride (383 mg, 1.81 mmol) was added in portions and the mixture was stirred at 25° C. for another 16 hours. The reaction mixture was adjusted to pH=8˜ 9 with saturated sodium bicarbonate aqueous solution (20 mL). The organic layer was separated and the aqueous layer was extracted with dichloromethane (10 mL) for three times. The combined organic phases were washed with brine (10 mL), dried over Na₂SO₄₍ₛ₎ and filtered. The filtrate was concentrated to give a residue, which was purified by C-18 (acetonitrile:water=20% to 70%) to give the title compound (120 mg, 90% purity from ¹H NMR, 79.6% yield) as colorless oil. LC-MS (ESI): mass calcd. for C₁₈H₂₈F₂N₂O₄ 374.2, m/z found 375.4 [M+H]⁺. ¹H NMR (400 MHz, CDCl₃) δ 4.45-4.31 (m, 1H), 3.90-3.59 (m, 5H), 3.47 (d, J=12.4 Hz, 1H), 3.39 (t, J=8.4 Hz, 1H), 3.02-2.97 (m, 1H), 2.58 (t, J=15.2 Hz, 1H), 2.37-2.09 (m, 3H), 1.86-1.73 (m, 1H), 1.46 (s, 9H), 0.61-0.54 (m, 1H), 0.49-0.41 (m, 2H), 0.36-0.29 (m, 1H).

### S15-2: 2-(1-(((cis)-4-(tert-Butoxycarbonyl)-6,6-difluorohexahydropyrrolo[3,2-b]pyrrol-1(2H)-yl)methyl)cyclopropyl)acetic acid

To a solution of S15-1 (120 mg, 90% purity, 0.288 mmol) in tetrahydrofuran (2 mL), methanol (2 mL) and water (1 mL) was added lithium hydroxide monohydrate (30 mg, 0.715 mmol). After stirred at 25° C. for 16 hours, the mixture was diluted with water (10 mL), and acidified to pH=5˜6 with 1 M hydrochloride aqueous solution. The aqueous layer was extracted with ethyl acetate (10 mL) for three times. The combined organic layers were washed with brine (10 mL), dried over Na₂SO₄₍ₛ₎ and filtered. The filtrate was concentrated to give the title compound (100 mg, 84% purity, 81% yield) as colorless oil. LC-MS (ESI): mass calcd. for C₁₇H₂₆F₂N₂O₄ 360.2, m/z found 361.2 [M+H]⁺.

### S15-3: (cis)-tert-Butyl 4-((1-(2-(allyloxy)-2-oxoethyl)cyclopropyl)methyl)-3,3-difluorohexahydropyrrolo[3,2-b]pyrrole-1(2H)-carboxylate

To a solution of S15-2 (100 mg, 84% purity, 0.233 mmol) in N,N-dimethylformamide (2 mL) was added potassium carbonate (65 mg, 0.47 mmol) and allyl bromide (43 mg, 0.355 mmol). After stirred at 25° C. for 16 hours, the reaction mixture was diluted with water (20 mL), and extracted with ethyl acetate (10 mL) for three times. The combined organic phases were washed with brine (10 mL), dried over Na₂SO₄₍ₛ₎ and filtered. The filtrate was concentrated to give a residue, which was purified by C-18 (acetonitrile:water=30% to 70%) to give the title compound (80 mg, 91% purity, 78% yield) as colorless oil. LC-MS (ESI): mass calcd. for C₂₀H₃₀F₂N₂O₄ 400.2, m/z found 401.5 [M+H]⁺.

### S15: Allyl 2-(1-(((cis)-6,6-difluorohexahydropyrrolo[3,2-b]pyrrol-1(2H)-yl)methyl)cyclopropyl)acetate hydrochloride

A solution of S15-3 (80 mg, 91% purity, 0.182 mmol) in 4 M hydrochloride in ethyl acetate (4 mL) was stirred at 25° C. for 1 hour. The reaction mixture was concentrated to give the title compound (75 mg, 76% purity, 93% yield) as white solids. LC-MS (ESI): mass calcd. for C₁₅H₂₂F₂N₂O₂ 300.2, m/z found 301.2 [M+H]⁺.

### Compound 49-A: ethyl (4S)-6-((4-((1-(2-(allyloxy)-2-oxoethyl)cyclopropyl)methyl)-3,3-difluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

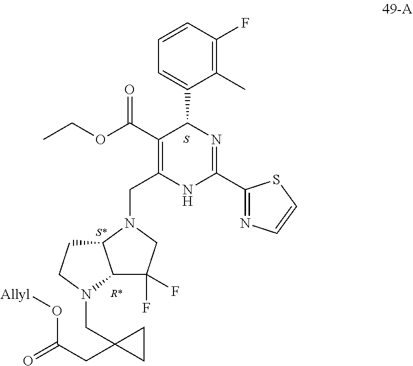

Compound 49-A was made from H2-1A with S15 according to typical coupling method 1. Purified by C-18 (acetonitrile:water=10% to 70%) to give the title compound (100 mg, 98.5% purity, 88.5% yield) as yellow solids. LC-MS (ESI): mass calcd. for C₃₃H₃₈F₃N₅O₄S 657.3, m/z found 658.3 [M+H]⁺.

### Compound 49: 2-(1-((4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)methyl)cyclopropyl)-acetic acid

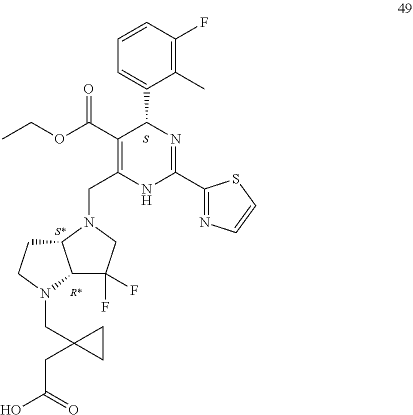

Compound 49 was made from compound 49-A using condition of typical method 2. Purified by Pre. HPLC (Column: Xbridge C18 (5 μm 19*150 mm), Mobile Phase A: water (+0.1% ammonium bicarbonate), Mobile Phase B: acetonitrile, UV: 254 nm, Flow rate: 15 mL/min, Gradient: 25-90% (% B)) to give the title compound (39.1 mg, 95.0% purity, 40.2% yield) as yellow solids. LC-MS (ESI): mass calcd. for C₃₀H₃₄F₃N₅O₄S 617.2, m/z found 618.3 [M+H]⁺. ¹H NMR (400 MHz, CDCl₃) δ 9.22 (s, 0.8H), 7.84 (d, J=3.2 Hz, 0.9H), 7.84 (d, J=2.8 Hz, 0.1H), 7.44-7.41 (m, 0.1H), 7.40 (d, J=3.2 Hz, 0.9H), 7.31-7.29 (m, 0.2H), 7.10-7.05 (m, 1H), 6.97 (d, J=6.8 Hz, 1H), 6.93-6.88 (m, 1H), 6.00 (s, 0.9H), 5.95 (s, 0.1H), 4.24 (d, J=17.2 Hz, 1H), 4.12 (d, J=16.8 Hz, 1H), 4.08-3.98 (m, 2H), 3.88-3.82 (m, 1H), 3.67-3.58 (m, 1H), 3.51-3.39 (m, 3H), 3.06-2.95 (m, 2H), 2.54-2.40 (m, 4H), 2.18-1.99 (m, 4H), 1.11 (t, J=7.2 Hz, 3H), 0.81-0.76 (m, 1H), 0.61-0.56 (m, 1H), 0.52-0.44 (m, 2H).

### Compound 50A: 4-((cis)-1-(((S)-5-(Ethoxycarbonyl)-6-(3-fluoro-2-methylphenyl)-2-(thiazol-2-yl)-3,6-dihydropyrimidin-4-yl)methyl)-3a-fluoro-4-oxohexahydropyrrolo[3,4-b]pyrrol-5(1H)-yl)-2,2-dimethylbutanoic acid

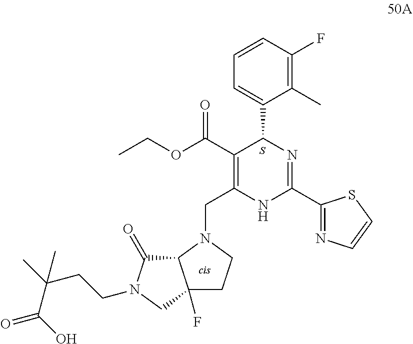

This compound was made from H2-1A and S52-A according to typical method 1 and 3. LC-MS (ESI): mass calcd. for C₃₀H₃₅F₂N₅O₅S 615.2, m/z found 616.3 [M+H]⁺. ¹H NMR (400 MHz, CD₃OD) δ 7.92 (d, J=3.2 Hz, 1H), 7.72 (d, J=2.8 Hz, 1H), 7.19-7.14 (m, 2H), 6.97-6.92 (m, 1H), 6.00 (s, 1H), 4.44 (d, J=18.8 Hz, 1H), 4.16 (d, J=16.8 Hz, 1H), 4.08 (q, J=7.2 Hz, 2H), 3.78-3.74 (m, 1H), 3.65-3.58 (m, 1H), 3.54-3.46 (m, 2H), 3.30-3.22 (m, 1H), 3.11-3.06 (m, 1H), 2.90-2.80 (m, 1H), 2.52 (s, 3H), 2.43-2.23 (m, 2H), 1.82-1.52 (m, 2H), 1.17-1.07 (m, 9H).

### Compound 51: ethyl (S)-6-(((cis)-3,3-difluoro-4-oxohexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate (Single Diastereomer)

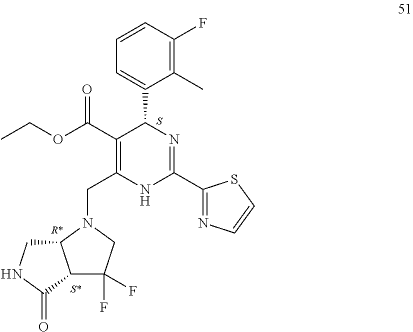

This compound was made using the procedure similar to Compound 42 by replacing tert-butyl 2,2-dimethyl-3-oxopropanoate with 4-methoxybenzaldehyde and replacing H5-1A with H2-1A. The protecting group PMB was removed using CF₃SO₃H (3.5 eq.)/TFA/DCM at 80° C. overnight. Purified by C18 column (acetonitrile:water=5% to 95%) and separated by Chiral Prep. HPLC (Column: Chiralpak IC 5 μm 20 mm*250 mm; Mobile Phase: Hex:EtOH=50:50 at 15 mL/min; Col. Temp: 30° C.; Wavelength: 214 nm) to afford the title compounds. LC-MS (ESI): mass calcd. for C₂₄H₂₄F₃N₅O₃S 519.5, m/z found 520.1 [M+H]⁺. ¹H NMR (400 MHz, CDCl₃) δ 9.13 (s, 1H), 7.84-7.81 (m, 1H), 7.41-7.38 (m, 1H), 7.09-7.04 (m, 1H), 6.98-6.89 (m, 2H), 6.02 (s, 1H), 5.72 (s, 1H), 4.41 (d, J=17.2 Hz, 1H), 4.09-3.90 (m, 4H), 3.61-3.39 (m, 4H), 2.99-2.90 (m, 1H), 2.53 (s, 3H), 1.12-1.08 (m, 3H).

### Compound 53A: 4-((cis)-1-(((S)-5-(ethoxycarbonyl)-6-(3-fluoro-2-methylphenyl)-2-(thiazol-2-yl)-3,6-dihydropyrimidin-4-yl)methyl)-3a-fluoro-6-oxohexahydropyrrolo[3,4-b]pyrrol-5(1H)-yl)-2,2-dimethylbutanoic acid

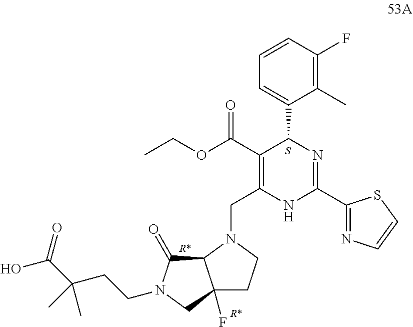

This compound was made from H2-1A and S52-B according to typical method 1 and 3. LC-MS (ESI): mass calcd. for C₃₀H₃₅F₂N₅O₅S 615.7, m/z found 616.2 [MH+]⁺. ¹H NMR (400 MHz, CD₃OD) δ 7.91-7.88 (m, 1H), 7.74-7.69 (m, 1H), 7.20-7.13 (m, 2H), 6.96-6.89 (m, 1H), 5.98-5.95 (m, 1H), 4.55 (d, J=17.2 Hz, 1H), 4.33 (d, J=17.2 Hz, 1H), 4.08-4.02 (m, 2H), 3.81-3.63 (m, 3H), 3.44-3.36 (m, 2H), 3.16-3.09 (m, 1H), 2.96-2.88 (m, 1H), 2.50 (s, 3H), 2.46-2.22 (m, 2H), 1.83-1.67 (m, 2H), 1.16-1.11 (m, 9H).

### Compound 54: 2-((cis)-1-(((S)-5-(Ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)pyrimidine-5-carboxylic acid (Single Diastereomer)

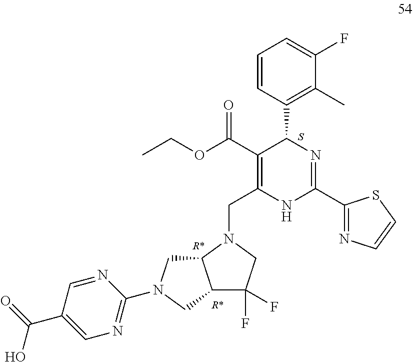

To a solution of Compound 179 (100 mg, 90% purity, 0.178 mmol) in N,N-dimethylformamide (5 mL) was added 2-chloropyrimidine-5-carboxylic acid (35 mg, 0.221 mmol) and N,N-diisopropylethylamine (70 mg, 0.542 mmol) at room temperature. After stirred at room temperature overnight, the mixture was diluted with dichloromethane (30 mL), washed with water (30 mL) twice, brine (30 mL), dried over Na₂SO₄₍ₛ₎, filtered and concentrated to give a residue, which was purified by C18 column (acetonitrile:water=5% to 95%) to give the title compound (55.0 mg, 99.4% purity, 49% yield) as yellow solids. LC-MS (ESI): mass calcd. for C₂₉H₂₈F₃N₇O₄S 627.2, m/z found 628.3 [M+H]⁺. ¹H NMR (400 MHz, DMSO-d₆) δ 9.27 (s, 0.7H), 8.75 (s, 0.3H), 8.62-8.39 (m, 2H), 8.00 (d, J=3.2 Hz, 0.15H), 7.98 (d, J=3.2 Hz, 0.15H), 7.75 (d, J=3.2 Hz, 0.85H), 7.69 (d, J=3.2 Hz, 0.85H), 7.31-7.30 (m, 0.85H), 7.25-7.20 (m, 1H), 7.17-7.12 (m, 0.15H), 7.07-7.01 (m, 1H), 5.84 (s, 0.85H), 5.76 (s, 0.15H), 4.27-4.20 (m, 2H), 4.18-4.11 (m, 1.6H), 4.00-3.95 (m, 2.4H), 3.91-3.88 (m, 1H), 3.81-3.75 (m, 1H), 3.58-3.49 (m, 2H), 3.24-3.15 (m, 2H), 2.42 (d, J=1.6 Hz, 2.5H), 2.39 (d, J=1.6 Hz, 0.5H), 1.06 (t, J=7.2 Hz, 3H).

### Compound 55: ethyl (S)-6-(((cis)-3,3-difluoro-6-oxohexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate (Single Diastereomer)

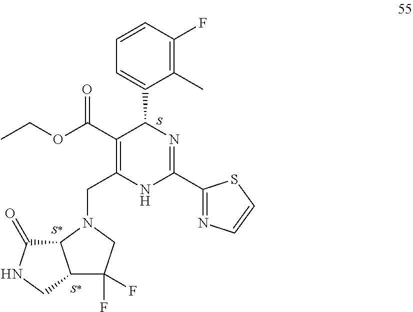

This compound was made together with compound 51. LC-MS (ESI): mass calcd. for C₂₄H₂₄F₃N₅O₃S 519.5, m/z found 520.1 [M+H]⁺. ¹H NMR (400 MHz, CDCl₃) δ 9.34 (s, 1H), 7.81 (d, J=3.2 Hz, 1H), 7.39 (d, J=3.2 Hz, 1H), 7.13-7.06 (m, 2H), 6.92-6.85 (m, 1H), 6.01 (s, 1H), 5.76 (s, 1H), 4.57-4.45 (m, 2H), 4.09-3.99 (m, 2H), 3.84-3.78 (m, 1H), 3.75-3.71 (m, 1H), 3.57-3.50 (m, 1H), 3.35-3.31 (m, 3H), 2.57-2.53 (m, 3H), 1.14 (t, J=7.2 Hz, 3H).

### Compound 66: 3-(((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)sulfonyl)propanoic acid (Single Diastereomer)

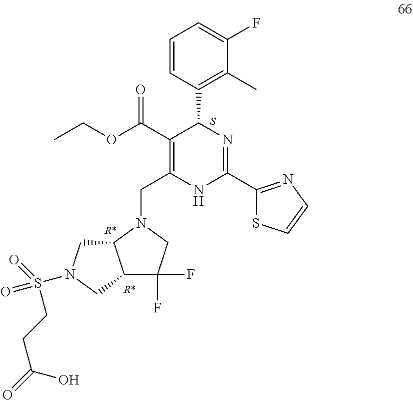

This compound was made using the procedure similar to Compound 46 by replacing tert-butyl 3-(chlorosulfonyl)-2,2-dimethylpropanoate with tert-butyl 3-(chlorosulfonyl)propanoate. Purified by C18 column (MeCN:water containing 0.5% HCOOH=1% to 50%) to give desired compound (10 mg, yield 19%) as a yellow solid. LCMS (ESI): mass calcd. For C₂₇H₃₀F₃N₅O₆S₂ 641.7, m/z found 642.2[M+H]⁺. ¹H NMR (400 MHz, DMSO-d₆) δ=9.63-9.40 (m, 1H), 8.07-7.87 (m, 2H), 7.29-7.13 (m, 1H), 7.12-6.99 (m, 2H), 5.89-5.76 (m, 1H), 4.30-4.09 (m, 2H), 4.05-3.94 (m, 3H), 3.94-3.84 (m, 1H), 3.67-3.46 (m, 3H), 3.44-3.39 (m, 2H), 3.24-3.03 (m, 1H), 2.70-2.56 (m, 3H), 2.45-2.30 (m, 4H), 1.13-0.99 (m, 3H).

### Compound 68A: 4-(4-(((S)-5-(ethoxycarbonyl)-6-(3-fluoro-2-methylphenyl)-2-(thiazol-2-yl)-3,6-dihydropyrimidin-4-yl)methyl)-6,6-difluoro-2-methylhexahydropyrrolo[3,2-c]pyrazol-1(2H)-yl)-2,2-dimethylbutanoic acid (Single Diastereomer)

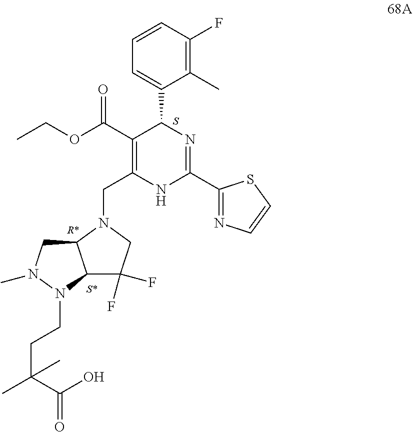

### Preparation of Intermediate S10

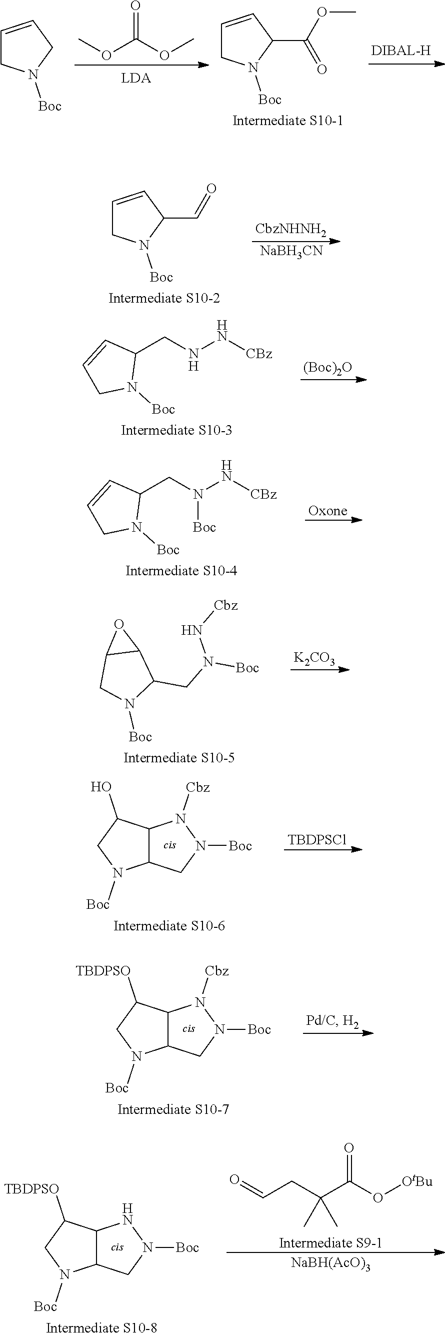

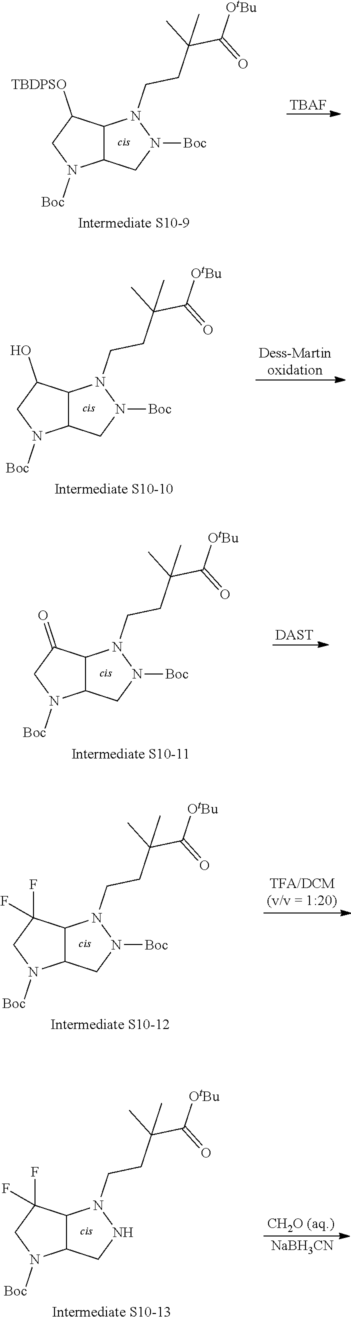

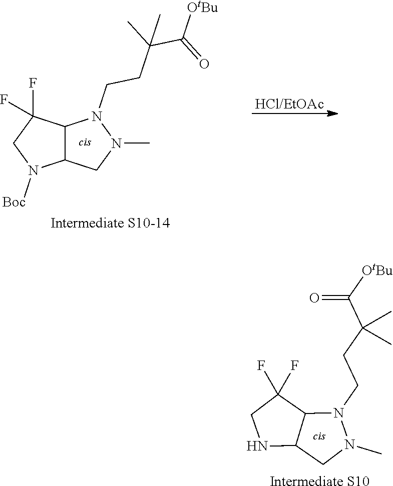

### S10-1: 1-tert-Butyl 2-methyl 1H-pyrrole-1,2(2H,5H)-dicarboxylate

To a solution of tert-butyl 2,5-dihydro-1H-pyrrole-1-carboxylate (50.0 g, 295 mmol) and dimethyl carbonate (32.0 g, 355 mmol) in tetrahydrofuran (1 L) was added 2 M lithium diisopropylamide in tetrahydrofuran (300 mL, 600 mmol) at −78° C. After stirred at 0° C. for 2 hours, the reaction mixture was poured into 1 N hydrochloride acid (1 L) and extracted with ethyl acetate (1 L) twice. The combined organic layers were washed with brine (1 L), dried over Na₂SO₄₍ₛ₎ and filtered. The filtrate was concentrated and purified by silica gel column chromatography (petroleum ether:ethyl acetate=20:1 to 1:1) to give the desired product. The procedure was repeated for two times to give the title compound (120 g, 90% purity from ¹H NMR, 80% yield) as yellow oil. ¹H NMR (400 MHz, CDCl₃) δ 6.04-5.96 (m, 1H), 5.79-5.72 (m, 1H), 5.08-4.97 (m, 1H), 4.36-4.18 (m, 2H), 3.77 (s, 1.5H), 3.76 (s, 1.5H), 1.51 (s, 4.5H), 1.46 (s, 4.5H).

### S10-2: tert-Butyl 2-formyl-2,5-dihydro-1H-pyrrole-1-carboxylate

To a solution of 1-tert-butyl 2-methyl 1H-pyrrole-1,2(2H,5H)-dicarboxylate S10-1 (40.0 g, 90% purity, 158 mmol) in dichloromethane (400 mL) was added 1.5 M diisobutylaluminum hydride in toluene (120 mL, 180 mmol) at −78° C. After stirred at −78° C. for 1 hour, the reaction mixture was quenched with methanol (40 mL) and saturated aqueous ammonium chloride solution (40 mL) in batches. Then celite was added and the mixture was warmed to room temperature and filtered. The filtrate was concentrated to give the desired product. The procedure was repeated for three times to give the title compound (120 g, 70% purity from ¹H NMR, 90% yield) as yellow oil. ¹H NMR (400 MHz, CDCl₃) δ 9.46 (d, J=3.6 Hz, 0.3H), 9.37 (d, J=4.4 Hz, 0.7H), 6.04-5.92 (m, 1H), 5.81-5.73 (m, 1H), 5.09-4.93 (m, 1H), 4.37-4.26 (m, 2H), 1.48 (s, 4.5H), 1.46 (s, 4.5H).

### S10-3: tert-Butyl 2-((2-((benzyloxy)carbonyl)hydrazinyl)methyl)-2,5-dihydro-1H-pyrrole-1-carboxylate

To a solution of tert-butyl 2-formyl-2,5-dihydro-1H-pyrrole-1-carboxylate S10-2 (10.4 g, 80% purity, 42.2 mmol) and benzyl hydrazinecarboxylate (9.17 g, 55.2 mmol) in methanol (120 mL) was added acetic acid (120 mL) at room temperature. After stirred at room temperature under nitrogen atmosphere for 2 hours, sodium cyanoborohydride (5.30 g, 84.3 mmol) was added at 0° C. and the mixture was stirred at room temperature overnight. Then it was concentrated to give a residue, which was diluted with dichloromethane (20 mL), basified with 5 M sodium hydroxide aqueous solution to pH=˜9, and extracted with dichloromethane (20 mL) for three times. The combined organic layers were dried over Na₂SO₄₍ₛ₎ and filtered. The filtrate was concentrated to give a residue, which was purified by silica gel column chromatography (petroleum ether:ethyl acetate=4:1 to 2:1) to give title compound (10.7 g, 80% purity from ¹H NMR, 58% yield) as colorless oil. LC-MS (ESI): mass calcd. for C₁₈H₂₅N₃O₄ 347.2, m/z found 348.2 [M+H]⁺. ¹H NMR (400 MHz, CDCl₃) δ 7.43-7.28 (m, 5H), 7.01 (s, 0.5H), 6.30 (s, 0.5H), 5.90-5.66 (m, 2H), 5.13 (s, 2H), 4.80-4.68 (m, 0.6H), 4.61-4.47 (m, 0.4H), 4.27-4.11 (m, 1.5H), 4.09-3.94 (m, 1.5H), 3.29-2.99 (m, 1.4H), 2.92-2.80 (m, 0.6H), 1.47 (s, 9H).

### S10-4: 2-Benzyl 1-tert-butyl 1-((1-(tert-butoxycarbonyl)-2,5-dihydro-1H-pyrrol-2-yl)methyl)hydrazine-1,2-dicarboxylate

To a solution of tert-butyl 2-((2-((benzyloxy)carbonyl)hydrazinyl)methyl)-2,5-dihydro-1H-pyrrole-1-carboxylate S10-3 (10.7 g, 80% purity, 24.6 mmol) and sodium hydroxide (1.32 g, 33.0 mmol) in 1,4-dioxane (213 mL) and water (32 mL) was added di-tert-butyl dicarbonate (9.20 g, 42.2 mmol) at room temperature. After stirred at room temperature under for 3 hours, another batch of sodium hydroxide (1.32 g, 33.0 mmol), water (32 mL) and di-tert-butyl dicarbonate (9.20 g, 42.2 mmol) were added. After stirred at 60° C. under nitrogen atmosphere overnight, the mixture was diluted with water (50 mL) and extracted with ethyl acetate (50 mL) for three times. The combined organic layers were dried over Na₂SO₄₍ₛ₎ and filtered. The filtrate was concentrated to give a residue, which was purified by silica gel column chromatography (petroleum ether:ethyl acetate=10:1 to 5:1) to give the title compound (11.9 g, 80% purity from ¹H NMR, 86% yield) as colorless oil. LC-MS (ESI): mass calcd. for C₂₃H₃₃N₃O₆ 447.2, m/z found 448.4 [M+H]⁺. ¹H NMR (400 MHz, CDCl₃) δ 7.66 (s, 0.5H), 7.52 (s, 0.5H), 7.42-7.28 (m, 5H), 5.87-5.64 (m, 2H), 5.25-5.05 (m, 2H), 4.83-4.71 (m, 0.7H), 4.64-4.56 (m, 0.3H), 4.30-4.18 (m, 1H), 4.07-3.92 (m, 1H), 3.75-3.41 (m, 2H), 1.44-1.32 (m, 18H).

### S10-5: 2-Benzyl 1-tert-butyl 1-((3-(tert-butoxycarbonyl)-6-oxa-3-azabicyclo[3.1.0]hexan-2-yl)methyl)hydrazine-1,2-dicarboxylate

To a solution of 2-benzyl 1-tert-butyl 1-((1-(tert-butoxycarbonyl)-2,5-dihydro-1H-pyrrol-2-yl)methyl)hydrazine-1,2-dicarboxylate S10-4 (5.00 g, 80% purity, 8.94 mmol) and disodium edetate dihydrate (170 mg, 0.457 mmol) in acetonitrile (150 mL) and water (100 mL) was added 1,1,1-trifluoroacetone (10 mL, 112 mmol) at 0° C. under nitrogen atmosphere. After stirred at 0° C. for 10 minutes, a mixture of potassium peroxymonosulfate (30.8 g, 50.1 mmol) and sodium bicarbonate (6.80 g, 80.9 mmol) were added over a period of 30 minutes. After stirred at room temperature overnight, the mixture was diluted with water (100 mL) and extracted with ethyl acetate (100 mL) for three times. The combined organic layers were washed with brine (200 mL), dried over Na₂SO₄₍ₛ₎, filtered and concentrated to give a residue, which was purified by silica gel column chromatography (petroleum ether:ethyl acetate=4:1 to 2:1) to give the title compound (4.67 g, 90% purity from ¹H NMR, 90% yield) as colorless oil. LC-MS (ESI): mass calcd. for C₂₃H₃₃N₃O₇ 463.2, m/z found 352.5 [M+H−112]⁺. ¹H NMR (400 MHz, CDCl₃) δ 7.66 (s, 1H), 7.43-7.32 (m, 5H), 5.32-5.08 (m, 2H), 4.39-4.29 (m, 0.6H), 4.20-4.15 (m, 0.4H), 3.96-3.75 (m, 2H), 3.72-3.52 (m, 2H), 3.48-3.45 (m, 1H), 3.40-3.15 (m, 1H), 1.46-1.30 (m, 18H).

### S10-6: 1-Benzyl 2,4-di-tert-butyl 6-hydroxytetrahydropyrrolo[3,2-c]pyrazole-1,2,4(5H)-tricarboxylate

To a solution of 2-benzyl 1-tert-butyl 1-((3-(tert-butoxycarbonyl)-6-oxa-3-azabicyclo[3.1.0]hexan-2-yl)methyl)hydrazine-1,2-dicarboxylate S10-5 (4.67 g, 90% purity, 9.07 mmol) in acetonitrile (200 mL) was added potassium carbonate (30.1 g, 218 mmol) at room temperature under nitrogen atmosphere. After stirred at 60° C. overnight, the mixture was concentrated to give a residue, which was purified by silica gel column chromatography (petroleum ether:ethyl acetate=4:1) to give the title compound (4.05 g, 90% purity from ¹H NMR, 87% yield) as white solids. LC-MS (ESI): mass calcd. for C₂₃H₃₃N₃O₇ 463.2, m/z found 308.3 [M+H−156]⁺. ¹H NMR (400 MHz, CDCl₃) δ 7.38-7.29 (m, 5H), 5.27 (d, J=12.4 Hz, 1H), 5.13 (d, J=12.4 Hz, 1H), 4.63-4.38 (m, 4H), 3.64 (d, J=12.4 Hz, 0.5H), 3.52 (d, J=12.4 Hz, 0.5H), 3.42-3.39 (m, 1H), 3.21-3.10 (m, 1H), 2.49 (br s, 1H), 1.49-1.41 (s, 18H).

### S10-7: 1-Benzyl 2,4-di-tert-butyl 6-((tert-butyldiphenylsilyl)oxy)tetrahydropyrrolo[3,2-c]pyrazole-1,2,4(5H)-tricarboxylate

To a solution of 1-benzyl 2,4-di-tert-butyl 6-hydroxytetrahydropyrrolo[3,2-c]pyrazole-1,2,4(5H)-tricarboxylate S10-6 (7.20 g, 95% purity, 14.8 mmol), 1H-imidazole (4.20 g, 61.7 mmol) and 4-dimethylaminopyridine (900 mg, 7.37 mmol) in dichloromethane (150 mL) was added tert-butylchlorodiphenylsilane (12.0 g, 43.7 mmol) at 0° C. After stirred at room temperature under nitrogen atmosphere for 3 hours, the mixture was diluted with water (100 mL) and extracted with dichloromethane (100 mL) for three times. The combined organic layers were dried over Na₂SO₄₍ₛ₎ and filtered. The filtrate was concentrated to give a residue, which was purified by silica gel column chromatography (petroleum ether:ethyl acetate=25:1 to 10:1) to give the title compound (10.1 g, 90% purity from ¹H NMR, 88% yield) as white solids. LC-MS (ESI): mass calcd. for C₃₉H₅₁N₃O₇Si 701.4, m/z found 702.9 [M+H]⁺. ¹H NMR (400 MHz, CDCl₃) δ 7.69-7.29 (m, 15H), 5.24-5.18 (m, 1H), 5.09-5.06 (m, 1H), 4.75-4.67 (m, 0.6H), 4.63-4.56 (m, 0.4H), 4.55-4.18 (m, 3H), 3.51 (d, J=12.4 Hz, 0.4H), 3.33 (d, J=12.0 Hz, 0.6H), 3.20-3.17 (m, 1H), 3.12-3.03 (m, 1H), 1.52-1.39 (m, 18H), 1.03 (s, 9H).

### S10-8: Di-tert-butyl 6-((tert-butyldiphenylsilyl)oxy)hexahydropyrrolo[3,2-c]pyrazole-2,4-dicarboxylate

To a solution of 1-benzyl 2,4-di-tert-butyl 6-((tert-butyldiphenylsilyl)oxy)tetrahydropyrrolo[3,2-c]pyrazole-1,2,4(5H)-tricarboxylate S10-7 (10.1 g, 90% purity, 13.0 mmol) in ethanol (100 mL) and 28% ammonia aqueous solution (0.2 mL) was added 10% palladium on charcoal wt. (3.0 g) at room temperature. After stirred at room temperature under hydrogen balloon for 3 hours, the mixture was filtered and the filtrate was concentrated under reduced pressure to give the title compound (8.0 g, 90% purity from ¹H NMR, 98% yield) as white solids. LC-MS (ESI): mass calcd. for C₃₁H₄₅N₃O₅Si 567.3, m/z found 568.5 [M+H]⁺. ¹H NMR (400 MHz, CDCl₃) δ 7.64-7.58 (m, 4H), 7.45-7.36 (m, 6H), 4.65-4.63 (m, 0.6H), 4.52-4.50 (m, 0.4H), 4.29 (s, 1H), 4.20 (d, J=12.4 Hz, 0.6H), 4.02 (d, J=12.0 Hz, 0.4H), 3.78-3.74 (m, 1H), 3.59 (d, J=12.0 Hz, 0.4H), 3.42 (d, J=12.4 Hz, 0.6H), 3.22-3.10 (m, 2H), 1.51 (s, 3.6H), 1.45 (s, 5.4H), 1.39 (s, 9H), 1.05 (s, 9H).

### S10-9: Di-tert-butyl 1-(4-(tert-butoxy)-3,3-dimethyl-4-oxobutyl)-6-((tert-butyldiphenylsilyl)oxy)hexahydropyrrolo[3,2-c]pyrazole-2,4-dicarboxylate

To a mixture of di-tert-butyl 6-((tert-butyldiphenylsilyl)oxy)hexahydropyrrolo[3,2-c]pyrazole-2,4-dicarboxylate S10-8 (5.00 g, 90% purity, 7.93 mmol) in 1,2-dichloroethane (50 mL) was added tert-butyl 2,2-dimethyl-4-oxopentanoate S9-1 (3.50 g, 90% purity, 16.9 mmol) and acetic acid glacial (5 mL). The reaction was heated to reflux for 3 hours. Then sodium triacetoxyborohydride (8.50 g, 40.1 mmol) was added by point wise. After stirred at room temperature for 3 hours, the mixture was diluted with water (150 mL) and extracted with dichloromethane (100 mL) for three times. The combined organic layers were dried over Na₂SO₄₍ₛ₎ and filtered. The filtrate was concentrated to give a residue, which was purified by silica gel column chromatography (petroleum ether:ethyl acetate=15:1 to 5:1) to give the title compound (5.40 g, 90% purity from ¹H NMR, 83% yield) as white solids. LC-MS (ESI): mass calcd. for C₄₁H₆₃N₃O₇Si 737.4, m/z found 738.4 [M+H]⁺. ¹H NMR (400 MHz, CDCl₃) δ 7.65-7.58 (m, 4H), 7.45-7.34 (m, 6H), 4.57-4.55 (m, 0.6H), 4.44-4.42 (m, 0.4H), 4.28-4.25 (m, 1.6H), 4.15-4.13 (m, 0.4H), 3.55 (d, J=11.6 Hz, 0.4H), 3.37 (d, J=11.6 Hz, 0.6H), 3.27-3.07 (m, 3H), 2.56-2.25 (m, 2H), 1.68-1.56 (m, 2H), 1.51 (s, 3.5H), 1.45 (s, 5.5H), 1.40-1.37 (m, 18H), 1.07-1.03 (m, 15H).

### S10-10: Di-tert-butyl 1-(4-(tert-butoxy)-3,3-dimethyl-4-oxobutyl)-6-hydroxyhexahydropyrrolo[3,2-c]pyrazole-2,4-dicarboxylate

To a solution of di-tert-butyl 1-(4-(tert-butoxy)-3,3-dimethyl-4-oxobutyl)-6-((tert-butyldiphenylsilyl)oxy)hexahydropyrrolo[3,2-c]pyrazole-2,4-dicarboxylate S10-9 (5.40 g, 90% purity, 6.59 mmol) in tetrahydrofuran (50 mL) was added 1 M tetrabutylammonium fluoride in tetrahydrofuran (20 mL, 20 mmol) by drop wise and the reaction mixture was stirred at room temperature for 2 hours. Then it was concentrated to afford a crude product, which was purified by silica gel column chromatography (petroleum ether:ethyl acetate=5:1 to 2:1) to give the title compound (3.60 g, 90% purity from ¹H NMR, 98% yield) as white solids. ¹H NMR (400 MHz, CDCl₃) δ 4.54-4.40 (m, 1H), 4.25-3.99 (m, 2H), 3.45-3.27 (m, 4H), 2.74-2.64 (m, 2H), 1.81-1.70 (m, 2H), 1.48-1.43 (m, 27H), 1.13 (s, 6H).

### S10-11: Di-tert-butyl 1-(4-(tert-butoxy)-3,3-dimethyl-4-oxobutyl)-6-oxohexahydropyrrolo[3,2-c]pyrazole-2,4-dicarboxylate

To a solution of di-tert-butyl 1-(4-(tert-butoxy)-3,3-dimethyl-4-oxobutyl)-6-hydroxyhexahydropyrrolo[3,2-c]pyrazole-2,4-dicarboxylate S10-10 (3.60 g, 90% purity, 6.49 mmol) in dichloromethane (100 mL) was added Dess-Martin periodinane (11.0 g, 25.9 mmol) at 0° C. under nitrogen atmosphere. After stirred at room temperature for 3 hours, saturated sodium bicarbonate aqueous solution (150 mL) was added and the reaction mixture was extracted with dichloromethane (100 mL) for three times. The combined organic layers were washed with brine (50 mL), dried Na₂SO₄₍ₛ₎ and filtered. The filtrate was concentrated to give a residue, which was purified by silica gel column chromatography (petroleum ether:ethyl acetate=10:1 to 3:1) to give the title compound (2.20 g, 90% purity from ¹H NMR, 61% yield) as yellow oil. ¹H NMR (400 MHz, CDCl₃) δ 4.81-4.66 (m, 1H), 4.43-4.30 (m, 1H), 3.79-3.73 (m, 2H), 3.54-3.45 (m, 1H), 3.38-3.38 (m, 1H), 2.75 (t, J=8.0 Hz, 2H), 1.86-1.73 (m, 2H), 1.50-1.43 (m, 27H), 1.13 (s, 6H).

### S10-12: Di-tert-butyl 1-(4-(tert-butoxy)-3,3-dimethyl-4-oxobutyl)-6,6-difluorohexahydropyrrolo[3,2-c]pyrazole-2,4-dicarboxylate

A solution of di-tert-butyl 1-(4-(tert-butoxy)-3,3-dimethyl-4-oxobutyl)-6-oxohexahydropyrrolo[3,2-c]pyrazole-2,4-dicarboxylate S10-11 (2.20 g, 90% purity, 3.98 mmol) in dichloromethane (50 mL) was added diethylaminosulfur trifluoride (3.0 mL, 22.7 mmol) at −78° C. After stirred at room temperature for 3 hours, the reaction mixture was added to the saturated aqueous sodium bicarbonate solution (100 mL). The two layers were separated and the aqueous phase was extracted with dichloromethane (100 mL). The combined organic extracts were washed with brine (100 mL), dried over Na₂SO₄₍ₛ₎, filtered and concentrated. The obtained residue was purified by silica gel column chromatography (petroleum ether:ethyl acetate=30:1 to 15:1) to give the title compound (1.70 g, 90% purity from ¹H NMR, 74% yield) as yellow oil. ¹H NMR (400 MHz, CDCl₃) δ 4.55-4.45 (m, 1H), 4.42-4.23 (m, 1H), 3.87-3.67 (m, 1H), 3.53-3.41 (m, 2H), 3.21-3.15 (m, 1H), 2.84-2.71 (m, 2H), 1.86-1.72 (m, 2H), 1.49-1.43 (m, 27H), 1.14 (s, 3H), 1.13 (s, 3H).

### S10-13: tert-Butyl 1-(4-(tert-butoxy)-3,3-dimethyl-4-oxobutyl)-6,6-difluorohexahydropyrrolo[3,2-c]pyrazole-4(2H)-carboxylate

The solution of (cis)-di-tert-butyl 1-(4-(tert-butoxy)-3,3-dimethyl-4-oxobutyl)-6,6-difluorohexahydropyrrolo[3,2-c]pyrazole-2,4-dicarboxylate S10-12 (1.10 g, 90% purity, 1.91 mmol) in trifluoroacetic acid (5 mL) and dichloromethane (100 mL) was stirred at 0° C. for 3 hours. Then it was poured into the saturated sodium bicarbonate aqueous solution (150 mL). The two layers were separated and the aqueous phase was extracted with dichloromethane (100 mL) twice. The combined organic extracts were washed with brine (100 mL), dried over Na₂SO₄₍ₛ₎, filtered and concentrated to give the crude product (900 mg, 70% purity from ¹H NMR, 79% yield) as yellow oil. ¹H NMR (400 MHz, CDCl₃) δ 4.62-4.54 (m, 1H), 3.97-3.78 (m, 1H), 3.59-3.48 (m, 1H), 3.38-3.34 (m, 1H), 3.27-2.23 (m, 0.5H), 3.13-3.11 (m, 0.5H), 3.02-2.99 (m, 1H), 2.75-2.63 (m, 2H), 1.83-1.76 (m, 2H), 1.47-1.43 (m, 18H), 1.15 (s, 3H), 1.14 (s, 3H).

### S10-14: tert-Butyl 1-(4-(tert-butoxy)-3,3-dimethyl-4-oxobutyl)-6,6-difluoro-2-methylhexahydropyrrolo[3,2-c]pyrazole-4(2H)-carboxylate

To a solution of tert-butyl 1-(4-(tert-butoxy)-3,3-dimethyl-4-oxobutyl)-6,6-difluorohexahydropyrrolo[3,2-c]pyrazole-4(2H)-carboxylate S10-13 (900 mg, 70% purity, 1.50 mmol) in methanol (10 mL) and acetic acid (1 mL) was added 37% formaldehyde aqueous solution (2 mL, 26.9 mmol). After stirred at room temperature for 0.5 hour, sodium cyanoborohydride (200 mg, 3.18 mmol) was added by point wise. The stirring was continued 0.5 hour, and water (50 mL) was added to the reaction mixture. The mixture was extracted with ethyl acetate (50 mL) twice. The combined organic phases were washed with saturated sodium bicarbonate aqueous solution (100 mL), brine (100 mL), dried over Na₂SO₄₍ₛ₎, filtered and concentrated to give the crude product, which was purified by C18 column (acetonitrile water (0.1% ammonium bicarbonate)=5% to 80%) to give the title compound (600 mg, 90% purity from ¹H NMR, 83% yield) as yellow oil. ¹H NMR (400 MHz, CDCl₃) δ 4.61-4.46 (m, 1H), 3.93-3.74 (m, 2H), 3.46-3.27 (m, 2H), 3.03 (d, J=12.0 Hz, 0.5H), 2.84 (d, J=12.0 Hz, 0.5H), 2.80-2.67 (m, 2H), 2.55 (s, 3H), 1.83-1.70 (m, 2H), 1.47-1.44 (m, 18H), 1.15 (s, 6H).

### S10: tert-Butyl 4-(6,6-difluoro-2-methylhexahydropyrrolo[3,2-c]pyrazol-1(2H)-yl)-2,2-dimethylbutanoate

A solution of tert-butyl 1-(4-(tert-butoxy)-3,3-dimethyl-4-oxobutyl)-6,6-difluoro-2-methylhexahydropyrrolo[3,2-c]pyrazole-4(2H)-carboxylate S10-14 (500 mg, 90% purity, 1.04 mmol) in 3 M hydrochloride in ethyl acetate (50 mL) was stirred at room temperature for 3 hours. The reaction mixture was poured into ice water (100 mL) and extracted with ethyl acetate (100 mL) twice. The aqueous phase was basified by saturated sodium bicarbonate aqueous solution to pH to 8-9 and extracted with dichloromethane (100 mL) twice. The combined organic extracts were washed with brine (100 mL), dried over Na₂SO₄₍ₛ₎, filtered and concentrated to give the compound (240 mg, 90% purity from ¹H NMR, 62% yield) as yellow oil. ¹H NMR (400 MHz, CDCl₃) δ 4.16 (q, J=8.0 Hz, 1H), 3.32-3.18 (m, 3H), 3.08 (t, J=13.6 Hz, 1H), 2.86-2.70 (m, 2H), 2.43 (s, 3H), 2.39-2.34 (m, 1H), 1.82-1.67 (m, 2H), 1.44 (s, 9H), 1.16-1.15 (m, 6H).

### Compound 68A-1: ethyl (4S)-6-((1-(4-(tert-butoxy)-3,3-dimethyl-4-oxobutyl)-6,6-difluoro-2-methylhexahydropyrrolo[3,2-c]pyrazol-4(1H)-yl)methyl)-4-(3-fluoro-2-methylphenyl)-2-thiazol-2-yl)-1,4-dihydropyrimidine-5-carboxylate

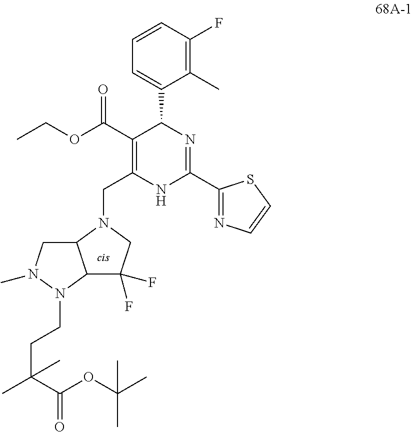 [SMILES: CCOC(=O)C1=C(CN2CC(F)(F)C3C2CN(C)N3CCC(C)(C)C(=O)OC(C)(C)C)NC(c2nccs2)=N[C@H]1c1cccc(F)c1C]

This compound was prepared from H2-1A and S10 according to Typical coupling method 1. LC-MS (ESI): mass calcd. for C₃₄H₄₅F₃N₆O₄S 690.3, m/z found 691.5 [M+H]⁺. ¹H NMR (400 MHz, CDCl₃) δ 9.17 (s, 1H), 7.78-7.76 (m, 1H), 7.40-7.39 (m, 1H), 7.11-7.05 (m, 1H), 6.99-6.96 (m, 1H), 6.91 (t, J=8.8 Hz, 1H), 6.10-6.00 (m, 1H), 4.28-4.13 (m, 2H), 4.10-3.91 (m, 3H), 3.68-3.43 (m, 2H), 3.33-3.13 (m, 1H), 3.08-2.92 (m, 1H), 2.89-2.67 (m, 5H), 2.60-2.33 (m, 4H), 1.84-1.71 (m, 2H), 1.44 (s, 9H), 1.16 (s, 6H), 1.14-1.09 (m, 3H).

Racemic compound 68A-1 (330 mg, 90% purity, 0.430 mmol) was separated by chiral HPLC (separation condition: Column: Superchiral IC 5 μm 20*250 mm; Mobile Phase: hexane:IPA=95:5 at 30 mL/min; Temp: 30° C.; Wavelength: 214 nm) to afford the title compounds compound 68A-2 (130 mg, 90% purity from ¹H NMR, 39% yield, 99.8% stereopure) and compound 68A-3 (110 mg, 90% purity from ¹H NMR, 33% yield, 99.3% stereopure) as yellow solids.

compound 68A-2: LC-MS (ESI): mass calcd. for C₃₄H₄₅F₃N₆O₄S 690.3, m/z found 691.5 [M+H]⁺. Chiral analysis (Chiral Column: Chiralpak IC 5 μm 4.6*250 mm; Mobile Phase: Hex:IPA=95:5 at 1 mL/min; Temp: 30° C.; Wavelength: 254 nm; R_{T}=9.385 min). ¹H NMR (400 MHz, CDCl₃) δ 9.17 (br s, 1H), 7.77 (d, J=2.8 Hz, 1H), 7.40 (d, J=3.2 Hz, 1H), 7.11-7.05 (m, 1H), 6.98 (d, J=7.6 Hz, 1H), 6.91 (t, J=8.8 Hz, 1H), 6.01 (s, 1H), 4.25 (d, J=16.8 Hz, 1H), 4.15 (d, J=17.2 Hz, 1H), 4.09-3.95 (m, 3H), 3.66-3.54 (m, 1H), 3.52-3.43 (m, 1H), 3.32-3.20 (m, 1H), 3.00-2.92 (m, 1H), 2.89-2.78 (m, 2H), 2.70 (br s, 4H), 2.54 (s, 3H), 1.85-1.71 (m, 2H), 1.45 (s, 9H), 1.16 (s, 6H), 1.11 (t, J=6.8 Hz, 3H).

compound 68A-3: LC-MS (ESI): mass calcd. for C₃₄H₄₅F₃N₆O₄S 690.32, m/z found 691.5 [M+H]⁺. Chiral analysis (Chiral Column: Chiralpak IC 5 μm 4.6*250 mm; Mobile Phase: Hex:IPA=95:5 at 1 mL/min; Temp: 30° C.; Wavelength: 254 nm; R_{T}=12.277 min). ¹H NMR (400 MHz, CDCl₃) δ 9.18 (br s, 1H), 7.76 (d, J=2.8 Hz, 1H), 7.40 (d, J=2.8 Hz, 1H), 7.11-7.05 (m, 1H), 6.97 (d, J=7.2 Hz, 1H), 6.91 (t, J=8.8 Hz, 1H), 6.00 (s, 1H), 4.26 (d, J=16.8 Hz, 1H), 4.15 (d, J=16.8 Hz, 1H), 4.10-3.92 (m, 3H), 3.64-3.47 (m, 2H), 3.27-3.15 (m, 1H), 3.05-2.97 (m, 1H), 2.90-2.72 (m, 2H), 2.66 (s, 4H), 2.54 (d, J=1.6 Hz, 3H), 1.83-1.70 (m, 2H), 1.45 (s, 9H), 1.16 (s, 6H), 1.12 (t, J=7.2 Hz, 3H).

### Compound 68A: 4-(4-(((S)-5-(Ethoxycarbonyl)-6-(3-fluoro-2-methylphenyl)-2-(thiazol-2-yl)-3,6-dihydropyrimidin-4-yl)methyl)-6,6-difluoro-2-methylhexahydropyrrolo[3,2-c]pyrazol-1(2H)-yl)-2,2-dimethylbutanoic acid

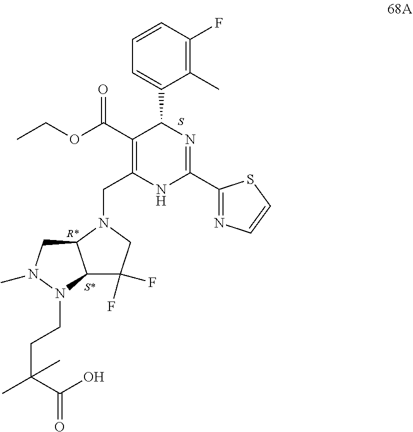

Compound 68A was prepared from compound 68A-2 according to typical method 3 to 15 remove tert-butyl ester and purified by C18 column (acetonitrile:water (0.1% ammonium bicarbonate=5% to 60%) to give the title compound (98 mg, 99.3% purity, 91% yield) as yellow solids. LC-MS (ESI): mass calcd. for C₃₀H₃₇F₃N₆O₄S 634.3, m/z found 635.3 [M+H]⁺. ¹H NMR (400 MHz, CDCl₃) δ 9.11 (s, 1H), 7.78 (d, J=2.8 Hz, 1H), 7.41 (d, J=3.2 Hz, 1H), 7.11-7.06 (m, 1H), 6.98 (d, J=7.6 Hz, 1H), 6.91 (t, J=9.2 Hz, 1H), 6.00 (s, 1H), 4.26 (d, J=17.2 Hz, 1H), 4.18-4.12 (m, 2H), 4.08-3.99 (m, 2H), 3.76-3.66 (m, 1H), 3.51-3.42 (m, 2H), 3.06-2.96 (m, 2H), 2.88-2.84 (m, 1H), 2.81-2.74 (m, 4H), 2.54 (s, 3H), 1.81 (t, J=7.6 Hz, 2H), 1.25 (s, 3H), 1.23 (s, 3H), 1.11 (t, J=7.2 Hz, 3H).

### Compound 69A: 4-(4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid

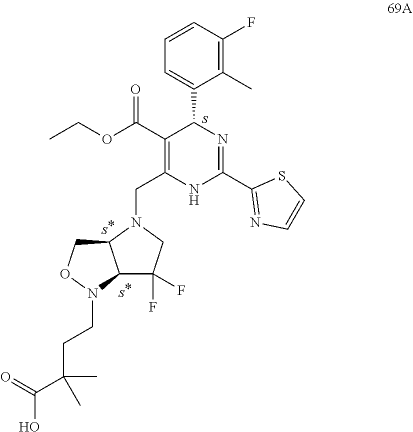

### Preparation of Intermediate S11

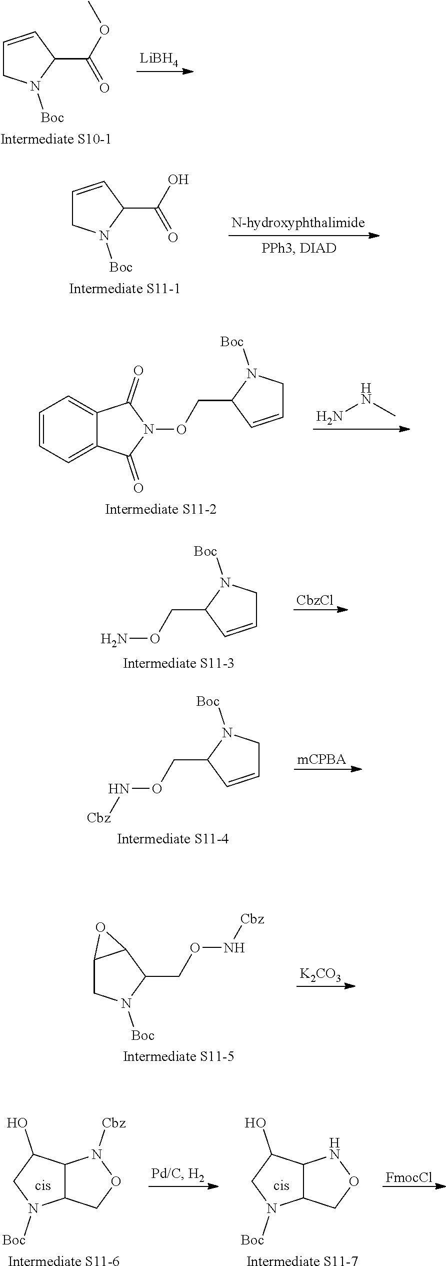

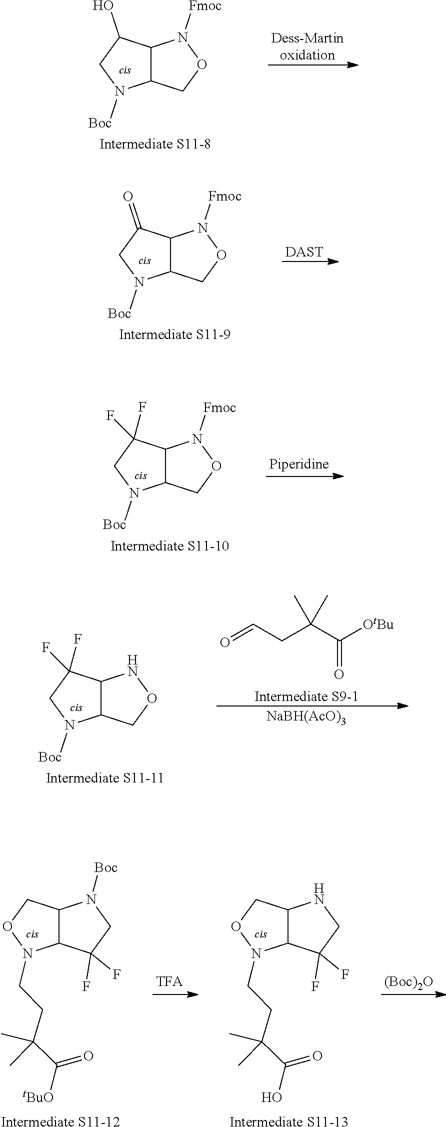

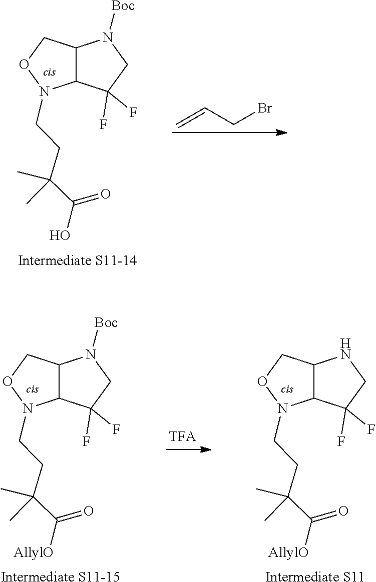

### S11-1: tert-butyl 2-(hydroxymethyl)-2,5-dihydro-1H-pyrrole-1-carboxylate

To a solution of 1-tert-butyl 2-methyl 1H-pyrrole-1,2(2H,5H)-dicarboxylate S10-1 (30 g, 90% purity, 119 mmol) in tetrahydrofuran (300 mL) was added lithium borohydride (7.80 g, 358 mmol) under ice bath. After stirred at room temperature for 12 hours the mixture was poured into water (800 mL) and extracted with ethyl acetate (350 mL) for three times. The combined organic layers were washed with brine (550 mL) and concentrated to get the desired compound (23 g, 100% purity from LCMS, 97% yield) as yellow oil. LC-MS (ESI): mass calcd. For C₁₀H₁₇NO₃ 199.12, m/z found 144.3 [M+H−56]⁺.

### S11-2: tert-butyl 2-(((1,3-dioxoisoindolin-2-yl)oxy)methyl)-2,5-dihydro-1H-pyrrole-1-carboxylate

To a solution of tert-butyl 2-(hydroxymethyl)-2,5-dihydro-1H-pyrrole-1-carboxylate S11-1 (23.0 g, 100% purity, 115 mmol), 2-hydroxyisoindoline-1,3-dione (19.0 g, 117 mmol) and triphenylphosphine (45.0 g, 172 mmol) in tetrahydrofuran (450 mL) was added diethyl azodicarboxylate (30.0 g, 173 mmol) under ice bath. After stirred at 25° C. for 12 hours the mixture was poured into water (950 mL) and extracted with ethyl acetate (450 mL) for three times. The combined organic layers were washed with brine (550 mL) and concentrated to get the crude. The crude was purified by column chromatography on silica gel (petroleum ether:ethyl acetate=4:1) to get the desired compound (40 g, 95% purity from NMR, 94.6% yield) as a yellow oil. ¹H NMR (400 MHz, CDCl₃) δ 7.90-7.76 (m, 4H), 6.20-6.14 (m, 1H), 6.00-5.94 (m, 1H), 4.91-4.80 (m, 1H), 4.69-4.54 (m, 1H), 4.36-4.10 (m, 3H), 1.49-1.48 (m, 9H).

### S11-3: tert-butyl 2-((aminooxy)methyl)-2,5-dihydro-1H-pyrrole-1-carboxylate

To a solution of tert-butyl 2-(((1,3-dioxoisoindolin-2-yl)oxy)methyl)-2,5-dihydro-1H-pyrrole-1-carboxylate S11-2 (40.0 g, 95% purity, 109 mmol) in dichloromethane (600 mL) was added 40% methylhydrazine aqueous solution (19.0 g, 165 mmol) under ice bath. After stirred at 0° C. for 1 hour the mixture was filtered and the filtrate was poured into water (450 mL) and extracted with dichloromethane (150 mL) for three times. The combined organic layers were washed with brine (550 mL) and concentrated to get the desired compound (27 g, 80% purity from LCMS, 92.3% yield) as a yellow oil. LC-MS (ESI): mass calcd. for C₁₀H₁₈N₂O₃ 214.1, m/z found 215.4 [M+H]⁺.

### S11-4: tert-butyl 2-(((((benzyloxy)carbonyl)amino)oxy)methyl)-2,5-dihydro-1H-pyrrole-1-carboxylate

To a mixture of tert-butyl 2-(2-aminoethyl)-2,5-dihydro-1H-pyrrole-1-carboxylate S11-3 (36.0 g, 80% purity, 134 mmol), sodium carbonate (28.0 g, 264 mmol) in tetrahydrofuran (400 mL) and water (100 mL) was added benzyl chloroformate (26.0 g, 152 mmol) dropwise unded ice bath. After stirred at 25° C. for 4 hours the mixture was poured into water (600 mL) and extracted with ethyl acetate (200 mL) for three times. The combined organic layers were washed with brine (400 mL) and concentrated to get the crude. The crude was purified by column chromatography on silica gel (petroleum ether:ethyl acetate=5:1) to get the desired compound (53 g, 86% purity from LCMS, 97.3% yield) as a yellow oil. LC-MS (ESI): mass calcd. for C₁₈H₂₄N₂O₅ 348.2, m/z found 349.4 [M+H]⁺.

### S11-5: tert-butyl 2-(((((benzyloxy)carbonyl)amino)oxy)methyl)-6-oxa-3-azabicyclo[3.1.0]hexane-3-carboxylate

To a solution of tert-butyl 2-(((((benzyloxy)carbonyl)amino)oxy)methyl)-2,5-dihydro-1H-pyrrole-1-carboxylate S11-4 (15.0 g, 86% purity, 37.0 mmol) in dichloromethane (300 mL) was added 3-chloroperoxybenzoic acid (16.0 g, 85% purity, 74.1 mmol). The mixture was stirred at room temperature for 16 hours. The reaction mixture was quenched by 2 M sodium bicarbonate aqueous solution (150 mL) and 2 M sodium thiosulfate aqueous solution (150 mL). The mixture was extracted with dichloromethane (300 mL) for three times. The combined organic layers were washed with brine (200 mL) and concentrated to get the crude. The crude was purified by column chromatography on silica gel (petroleum ether:ethyl acetate=3:1) to get the desired compound (13 g, 90% purity from LCMS, 86.7% yield) as a yellow oil. LC-MS (ESI): mass calcd. for C₁₈H₂₄N₂O₆ 364.2, m/z found 265.3 [M+H−100]⁺.

### S11-6: 1-benzyl 4-tert-butyl 6-hydroxytetrahydro-1H-pyrrolo[3,2-c]isoxazole-1,4(5H)-dicarboxylate

To a solution of tert-butyl 2-(((((benzyloxy)carbonyl)amino)oxy)methyl)-6-oxa-3-azabicyclo[3.1.0]hexane-3-carboxylate S11-5 (26.0 g, 90% purity, 71.4 mmol) in acetonitrile (520 mL) was added potassium carbonate (40.0 g, 289 mmol). After stirred at 25° C. for 12 hours the mixture was poured into water (200 mL) and extracted with ethyl acetate (200 mL) for three times. The combined organic layers were washed with brine (200 mL) and concentrated to get the crude. The crude was purified by silica gel chromatography (petroleum ether:ethyl acetate=3:1) to get the desired compound (20 g, 90% purity from NMR, 76% yield) as a yellow oil. ¹H NMR (400 MHz, CDCl₃) δ 7.39-7.32 (m, 5H), 5.22 (s, 2H), 4.87-4.72 (m, 1H), 4.62-4.55 (m, 1H), 4.37-4.36 (m, 1H), 4.27-4.24 (m, 0.5H), 3.75-3.69 (m, 2H), 3.62-3.57 (m, 0.5H), 3.52-3.48 (m, 1H), 1.46 (s, 9H).

### S11-7: tert-butyl 6-hydroxytetrahydro-1H-pyrrolo[3,2-c]isoxazole-4(5H)-carboxylate

A mixture of 1-benzyl 4-tert-butyl 6-hydroxytetrahydro-1H-pyrrolo[3,2-c]isoxazole-1,4(5H)-dicarboxylate S11-6 (20 g, 90% purity, 55 mmol) and 10% wt. palladium on charcoal (2 g) in ethanol (400 mL) was stirred under hydrogen balloon at 0° C. for 1.5 hours. The mixture was filtered and the filtrate was concentrated to get the desired compound (12 g, 90% purity from NMR, 95% yield) as a yellow oil. ¹H NMR (400 MHz, CDCl₃) δ 4.69-4.59 (m, 1H), 4.17-4.01 (m, 3H), 3.93-3.87 (m, 2H), 3.49-3.29 (m, 3H), 1.38-1.35 (m, 9H).

### S11-8: 1-((9H-fluoren-9-yl)methyl) 4-tert-butyl 6-hydroxytetrahydro-1H-pyrrolo[3,2-c]isoxazole-1,4(5H)-dicarboxylate

To a mixture of tert-butyl 6-hydroxytetrahydro-1H-pyrrolo[3,2-c]isoxazole-4(5H)-carboxylate S11-7 (12.0 g, 90% purity, 47.0 mmol), sodium bicarbonate (22.0 g, 226 mmol) in tetrahydrofuran (180 mL) and water (70 mL) was added (9H-fluoren-9-yl)methyl carbonochloridate (13.0 g, 52.1 mmol). After stirred at 25° C. for 4 hours the mixture was poured into water (200 mL) and extracted with ethyl acetate (200 mL) for three times. The combined organic layers were washed with brine (200 mL) and concentrated to get the crude. The crude was purified by column chromatography on silica gel (petroleum ether:ethyl acetate=4:1) to get the desired compound (21 g, 100% purity from LCMS, 98% yield) as yellow oil. LC-MS (ESI): mass calcd. for C₂₅H₂₈N₂O₆ 452.2, m/z found 397.3 [M+H−56]⁺.

### S11-9: 1-((9H-fluoren-9-yl)methyl) 4-tert-butyl 6-oxotetrahydro-1H-pyrrolo[3,2-c]isoxazole-1,4(5H)-dicarboxylate

To a solution of 1-((9H-fluoren-9-yl)methyl) 4-tert-butyl 6-hydroxytetrahydro-1H-pyrrolo[3,2-c]isoxazole-1,4(5H)-dicarboxylate S11-8 (21.0 g, 100% purity, 46.5 mmol) in dichloromethane (420 mL) was added Dess-Martin periodinane (39.4 g, 92.9 mmol). After stirred at 25° C. for 5 hours the reaction mixture was quenched with 2 M sodium bicarbonate aqueous solution (500 mL) and 2 M sodium thiosulfate aqueous solution (500 mL). The mixture was extracted with dichloromethane (300 mL) for three times. The combined organic layers were washed with brine (300 mL) and concentrated to get the crude. The crude was purified by column chromatography on silica gel (petroleum ether:ethyl acetate=4:1) to get the desired compound (18.7 g, 90% purity from NMR, 99% yield) as white solids. ¹H NMR (400 MHz, CDCl₃) δ 7.78-7.76 (m, 2H), 7.64-7.61 (m, 2H), 7.44-7.40 (m, 2H), 7.35-7.31 (m, 2H), 4.87-4.59 (m, 3H), 4.57-4.47 (m, 1H), 4.34-4.23 (m, 2H), 3.96-3.82 (m, 2H), 3.60-3.53 (m, 1H) 1.52-1.49 (m, 9H).

### S11-10:1-((9H-fluoren-9-yl)methyl) 4-tert-butyl 6,6-difluorotetrahydro-1H-pyrrolo[3,2-c]isoxazole-1,4(5H)-dicarboxylate

To a solution of 1-((9H-fluoren-9-yl)methyl) 4-tert-butyl 6-oxotetrahydro-1H-pyrrolo[3,2-c]isoxazole-1,4(5H)-dicarboxylate S11-9 (6.50 g, 90% purity, 14.4 mmol) in dichloromethane (100 mL) was added diethylaminosulfurtrifluoride (23.1 g, 144 mmol) under 0° C. The mixture was stirred at 40° C. for 16 hours. The mixture was poured into 2 M sodium bicarbonate aqueous solution (500 mL) and extracted with dichloromethane (100 mL) for three times. The combined organic layers were washed with brine (100 mL) and concentrated to get the crude. The crude was purified by column chromatography on silica gel (petroleum ether:ethyl acetate=8:1) to get the desired compound (5.1 g, 88% purity from LCMS, 66% yield) as a yellow oil. LC-MS (ESI): mass calcd. for C₂₅H₂₆F₂N₂O₅ 472.2, m/z found 473.4 [M+H]⁺.

Chiral separation of S11-10: chiral Prep. SFC (Column: Chiralpak IG 5 μm 20*250 mm; Mobile Phase: CO₂:MeOH=60:40 at 45 g/min; Col. Temp: 40° C.; Wavelength: 214 nm, Back pressure: 100 bar), both yellow oil.

S11-10A: Chiral analysis (Column: Chiralpak IG 5 μm 4.6*250 mm; Mobile Phase: CO₂:EtOH=60:40 at 3 g/min; Col. Temp: 40° C.; Wavelength: 214 nm, Back pressure: 100 bar, R_{T}=2.29 min). ¹H NMR (300 MHz, CDCl₃) δ 7.82 (d, J=7.2 Hz, 2H), 7.67 (t, J=6.6 Hz, 2H), 7.49-7.44 (m, 2H), 7.40-7.37 (m, 2H), 4.88-4.79 (m, 1H), 4.73-4.53 (m, 3H), 4.35-4.28 (m, 1H), 4.21-4.12 (m, 1H), 4.06-3.93 (m, 1H), 3.67-3.50 (m, 2H), 1.54-1.53 (m, 9H).

S11-10B: Chiral analysis (Column: Chiralpak IA 5 μm 4.6*250 mm; Mobile Phase: CO₂:EtOH=70:30 at 3 g/min; Col. Temp: 40° C.; Wavelength: 214 nm, Back pressure: 100 bar, R_{T}=3.60 min). ¹H NMR (400 MHz, CDCl₃) δ 7.77 (d, J=7.6 Hz, 2H), 7.62 (t, J=7.6 Hz, 2H), 7.43-7.40 (m, 2H), 7.35-7.30 (m, 2H), 4.83-4.74 (m, 1H), 4.65-4.49 (m, 3H), 4.29-4.23 (m, 1H), 4.15-4.09 (m, 1H), 4.06-3.87 (m, 1H), 3.60-3.46 (m, 2H), 1.49-1.48 (m, 9H).

### S11-11: tert-butyl 6,6-difluorotetrahydro-1H-pyrrolo[3,2-c]isoxazole-4(5H)-carboxylate

To a solution of 1-((9H-fluoren-9-yl)methyl) 4-tert-butyl 6,6-difluorotetrahydro-1H-pyrrolo[3,2-c]isoxazole-1,4(5H)-dicarboxylate S11-10 (5.1 g, 88% purity, 9.5 mmol) in N,N-dimethylformide (40 mL) was added piperidine (4.00 g, 47.0 mmol). After stirred at room temperature for 3 hours the mixture was poured water (150 mL) and extracted with dichloromethane (30 mL) for three times. The combined organic layers were washed with brine (100 mL) and concentrated to get the crude. The crude was purified by column chromatography on silica gel (petroleum ether:ethyl acetate=4:1) to get the desired compound (2.3 g, 90% purity from NMR, 87.1% yield) as a yellow solid. ¹H NMR (400 MHz, CDCl₃) δ 5.55 (s, 1H), 4.94-4.92 (m, 0.6H), 4.84-4.82 (m, 0.4H), 4.33-4.31 (m, 0.6H), 4.22-4.19 (m, 0.4H), 4.09-3.88 (m, 2H), 3.58-3.47 (m, 2H), 1.48 (s, 9H).

### S11-12: tert-butyl 1-(4-(tert-butoxy)-3,3-dimethyl-4-oxobutyl)-6,6-difluorotetrahydro-1H-pyrrolo[3,2-c]isoxazole-4(5H)-carboxylate

To a solution of tert-butyl 6,6-difluorotetrahydro-1H-pyrrolo[3,2-c]isoxazole-4(5H)-carboxylate S11-11 (600 mg, 90% purity, 2.20 mmol) and tert-butyl 2,2-dimethyl-4-oxobutanoate (610 mg, 90% purity, 3.3 mmol) in dichloromethane (6 mL) was added 1.0 M titanium(IV) chloride tripropan-2-olate in dichloromethane (2.1 mL, 2.1 mmol). After the mixture was stirred at room temperature for 0.5 hours sodium triacetoxyborohydride (2.30 g, 10.8 mmol) and glacial acetic acid (1 mL) was added. After addition the mixture was stirred at room temperature for 4 hours. The mixture was poured into water (100 mL) and extracted with dichloromethane (50 mL) for three times. The combined organic layers were washed with brine (100 mL) and concentrated to get the crude. The crude was purified by column chromatography on silica gel (petroleum ether:ethyl acetate=10:1) to get the desired compound (749 mg, 90% purity from NMR, 74.3% yield) as a yellow oil. ¹H NMR (300 MHz, CDCl₃) δ 4.90-4.78 (m, 1H), 4.19-4.15 (m, 1H), 4.13-3.65 (m, 3H), 3.51-3.40 (m, 1H), 2.91-2.84 (m, 2H), 1.97-1.84 (m, 2H), 1.50 (s, 9H), 1.47 (s, 9H), 1.24-1.19 (m, 6H).

### S11-13: 4-(6,6-difluorohexahydro-1H-pyrrolo[3,2-c]isoxazol-1-yl)-2,2-dimethylbutanoic acid

To a solution tert-butyl 1-(4-(tert-butoxy)-3,3-dimethyl-4-oxobutyl)-6,6-difluorotetrahydro-1H-pyrrolo[3,2-c]isoxazole-4(5H)-carboxylate S11-12 (740 mg, 90% purity, 1.58 mmol) in dichloromethane (4 mL) was added 2,2,2-trifluoroacetic acid (8 mL). After stirred at room temperature for 3 hours the mixture was concentrated to get the desired compound (500 mg, 90% purity from TLC, 75.1% yield) as a yellow oil which was used for next step directly.

### S11-14: 4-(4-(tert-butoxycarbonyl)-6,6-difluorohexahydro-1H-pyrrolo[3,2-c]isoxazol-1-yl)-2,2-dimethylbutanoic acid

To a solution of 4-(6,6-difluorohexahydro-1H-pyrrolo[3,2-c]isoxazol-1-yl)-2,2-dimethylbutanoic acid S11-13 (500 mg, 90% purity, 1.2 mmol), di-tert-butyl dicarbonate (390 mg, 1.80 mmol) in tetrahydrofuran (8 mL) and water (4 mL) was added sodium bicarbonate (500 mg, 6.00 mmol). After stirred at room temperature for 12 hours the mixture was poured into water (30 mL) and extracted with dichloromethane (20 mL) for three times. The combined organic layers were washed with brine (30 mL) and concentrated to get the crude. The crude was purified by column chromatography on silica gel (petroleum ether:ethyl acetate=3:1) to get the desired compound (440 mg, 90% purity from NMR, 91.3% yield) as a yellow oil. ¹H NMR (400 MHz, CDCl₃) δ 4.86-4.74 (m, 1H), 4.18-4.11 (m, 1H), 4.08-3.85 (m, 1.5H), 3.77-3.60 (m, 1.5H), 3.48-3.37 (m, 1H), 2.99-2.78 (m, 2H), 2.01-1.85 (m, 2H), 1.46 (s, 9H), 1.26 (s, 6H).

### S11-15: tert-butyl 1-(4-(allyloxy)-3,3-dimethyl-4-oxobutyl)-6,6-difluorotetrahydro-1H-pyrrolo[3,2-c]isoxazole-4(5H)-carboxylate

To a mixture of 4-(4-(tert-butoxycarbonyl)-6,6-difluorohexahydro-1H-pyrrolo[3,2-c]isoxazol-1-yl)-2,2-dimethylbutanoic acid S11-14 (440 mg, 90% purity, 1.09 mmol) and potassium carbonate (600 mg, 4.34 mmol) in N,N-dimethylformamide (3 mL) was added allyl bromide (393 mg, 3.25 mmol) at room temperature. After addition, the reaction mixture was stirred at room temperature overnight. The reaction mixture was poured into water (30 mL) and extracted with ethyl acetate (60 mL) twice. The combined organic phases were washed with brine (30 mL), dried over sodium sulfate and filtered. The filtrate was concentrated under vacuum. The residue was purified by column chromatography on silica gel (petroleum ether:ethyl acetate=10:1) to give the desired compound (450 mg, 90% purity from HNMR, 92% yield) as a colorless oil. ¹H NMR (400 MHz, CDCl₃) δ 5.96-5.86 (m, 1H), 5.34-5.21 (m, 2H), 4.85-4.72 (m, 1H), 4.57-4.55 (m, 2H), 4.13-4.09 (m, 1H), 4.02-3.60 (m, 3H), 3.49-3.36 (m, 1H), 2.88-2.80 (m, 2H), 1.92-1.89 (m, 2H), 1.46 (s, 9H), 1.23 (s, 3H), 1.22 (s, 3H).

### S11: allyl 4-(6,6-difluorohexahydro-1H-pyrrolo[3,2-c]isoxazol-1-yl)-2,2-dimethylbutanoate 2,2,2-trifluoroacetate

To a solution of tert-butyl 1-(4-(allyloxy)-3,3-dimethyl-4-oxobutyl)-6,6-difluorotetrahydro-1H-pyrrolo[3,2-c]isoxazole-4(5H)-carboxylate S11-15 (450 mg, 90% purity, 1.00 mmol) in dichloromethane (2 mL) was added trifluoroacetic acid (4 mL) at room temperature. After addition the reaction mixture was stirred at room temperature for 0.5 hour. The reaction mixture was concentrated under vacuum to give the desired compound (465 mg, 90% purity from NMR, 100% yield) as yellow oil. The yellow oil was used to next step without purification. ¹H NMR (400 MHz, DMSO-d₆) δ 5.97-5.87 (m, 1H), 5.33-5.20 (m, 2H), 4.91-4.87 (m, 1H), 4.55-4.53 (m, 2H), 4.17-4.05 (m, 3H), 3.87-3.80 (m, 1H), 3.49-3.37 (m, 1H), 2.89-2.68 (m, 2H), 1.79-1.72 (m, 2H), 1.15 (s, 6H).

### Compound 69A-1: ethyl (4S)-6-((1-(4-(allyloxy)-3,3-dimethyl-4-oxobutyl)-6,6-difluorohexahydro-4H-pyrrolo[3,2-c]isoxazol-4-yl)methyl)-4-(3-fluoro-2-methylphenyl)-2-(thiazol-2-yl)-1,4-dihydropyrimidine-5-carboxylate

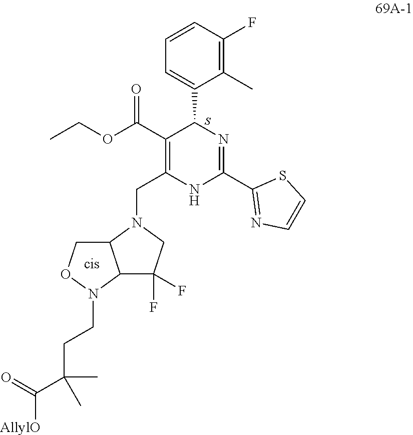

This compound was prepared from H2-1A and S11 according to Typical coupling method 1. LC-MS (ESI): mass calcd. for C₃₂H₃₈F₃N₅O₅S 661.7, m/z found 662.5 [M+H]⁺.

Compound 69A-1 (430 mg, 0.650 mmol, 100% purity) was separated by chiral prep. HPLC (separation condition: Column: Chiralpak IA 5 μm 20*250 mm; Mobile Phase: hexane:IPA:DEA=90:10:0.3 at 20 mL/min; Temp: 30° C.; Wavelength: 254 nm) to give the desired compound 69A-3 (190 mg, 95% purity by NMR, 100% ee, 42% yield) as yellow solids and 69A-2 (195 mg, 95% purity by NMR, 99.7% ee, 43% yield) as yellow solids.

Compound 69A-2 Chiral analysis (R_{T}=10.512 min, Area %: 99.8, Method: Column: Chiralpak IA 5 um 4.6*250 mm; Mobile Phase: hexane:IPA:DEA=90:10:0.2 at 1 mL/min; Temp: 30° C.; Wavelength: 254 nm). ¹H NMR (400 MHz, CDCl₃) δ 9.22 (br s, 1H), 7.81 (d, J=3.2 Hz, 1H), 7.41 (d, J=3.2 Hz, 1H), 7.10-7.05 (m, 1H), 6.98-6.96 (m, 1H), 6.93-6.88 (m, 1H), 6.00 (s, 1H), 5.95-5.88 (m, 1H), 5.34-5.30 (m, 1H), 5.24-5.21 (m, 1H), 4.59-4.56 (m, 2H), 4.32-4.20 (m, 3H), 4.06-3.99 (m, 4H), 3.62-3.57 (m, 1H), 3.47-3.37 (m, 1H), 3.12-3.06 (m, 1H), 2.87-2.73 (m, 2H), 2.54 (s, 3H), 1.95-1.89 (m, 2H), 1.24 (s, 3H), 1.30 (s, 3H), 1.12 (t, J=7.2 Hz, 3H).

Compound 69A-3 Chiral analysis (Method: Column: Chiralpak IA 5 um 4.6*250 mm; Mobile Phase: hexane:IPA:DEA=90:10:0.2 at 1 mL/min; Temp: 30° C.; Wavelength: 254 nm). ¹H NMR (400 MHz, CDCl₃) δ 9.26 (br s, 1H), 7.82 (d, J=3.2 Hz, 1H), 7.43 (d, J=3.2 Hz, 1H), 7.10-7.04 (m, 1H), 6.99-6.97 (m, 1H), 6.93-6.88 (m, 1H), 6.02 (s, 1H), 5.96-5.86 (m, 1H), 5.34-5.29 (m, 1H), 5.24-5.21 (m, 1H), 4.58-4.56 (m, 2H), 4.49-4.44 (m, 1H), 4.19-4.15 (m, 1H), 4.09-3.95 (m, 5H), 3.59-3.53 (m, 1H), 3.51-3.41 (m, 1H), 3.13-3.07 (m, 1H), 2.84-2.72 (m, 2H), 2.54 (s, 3H), 1.94-1.87 (m, 2H), 1.24 (s, 3H), 1.22 (s, 3H), 1.12 (t, J=7.2 Hz, 3H).

### Compound 69A: 4-(4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

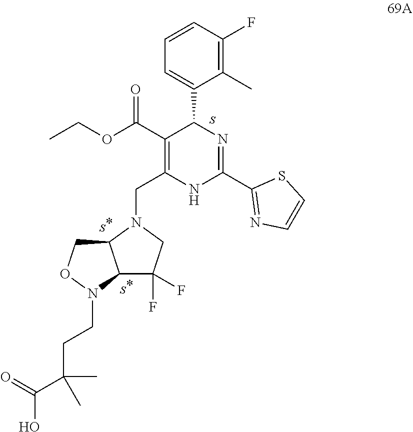

This compound was prepared from compound 69A-2 using typical method 2 to remove ally protection and purified by prep. HPLC (Column: SunFire C18 (5 μm 19*150 mm), Mobile phase A: water (0.1% ammonium bicarbonate), Mobile phase B: acetonitrile, UV: 214 nm, Flow rate: 15 mL/min, Gradient: 05-95% (% B)) to give the desired compound (60.6 mg, 99.4% purity from LCMS, 45% yield) as a yellow solid.

LC-MS (ESI): mass calcd. for C₂₉H₃₄F₃N₅O₅S 621.2, m/z found 621.8 [M+H]⁺. ¹H NMR (400 MHz, CDCl₃): δ 9.20 (s, 1H), 7.82 (d, J=3.2 Hz, 1H), 7.41 (d, J=3.2 Hz, 1H), 7.10-7.05 (m, 1H), 6.98-6.88 (m, 2H), 6.00 (s, 1H), 4.23-4.21 (m, 3H), 4.08-3.97 (m, 4H), 3.62-3.57 (m, 1H), 3.50-3.40 (m, 1H), 3.09 (t, J=12.8 Hz, 1H), 2.90-2.84 (m, 2H), 2.53 (s, 3H), 1.99-1.85 (m, 2H), 1.27 (s, 3H), 1.25 (s, 3H), 1.11 (t, J=7.2 Hz, 3H).

### Compound 70: 4-(2-acetyl-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

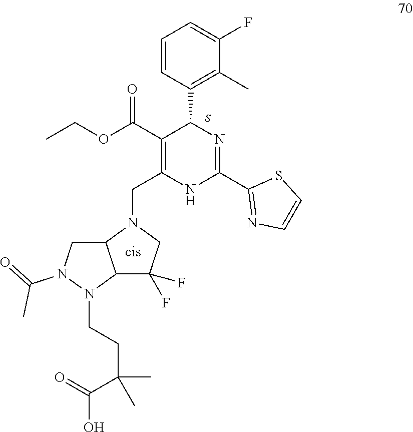

### Preparation of Intermediate S12

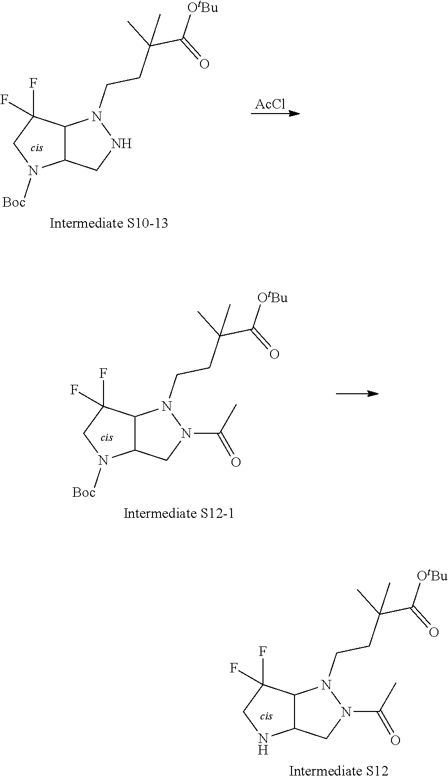

### S12-1: tert-butyl 2-acetyl-1-(4-(tert-butoxy)-3,3-dimethyl-4-oxobutyl)-6,6-difluorohexahydropyrrolo[3,2-c]pyrazole-4(1H)-carboxylate

To the solution of tert-butyl 1-(4-(tert-butoxy)-3,3-dimethyl-4-oxobutyl)-6,6-difluorohexahydropyrrolo[3,2-c]pyrazole-4(1H)-carboxylate S10-13 (68 mg, 0.16 mmol) and TEA (0.09 mL, 0.73 g/mL, 0.65 mmol) in DCM (5 mL, 1.33 g/mL, 78.3 mmol) while stirring at 0° C. was added Acetyl Chloride (15.27 mg, 0.19 mmol). Then the mixture was stirred at 20° C. for 2 hrs. To the mixture was added 15 mL of water extracted with DCM. The organic layers were collected, washed with brine, dried over anhydrous Na₂SO₄, filtered, and concentrated under reduced pressure to give crude product (70 mg) as colorless oil, which was used in next step directly.

### S12: tert-butyl 4-(2-acetyl-6,6-difluorohexahydropyrrolo[3,2-c]pyrazol-1(2H)-yl)-2,2-dimethylbutanoate

To the solution of tert-butyl 2-acetyl-1-(4-(tert-butoxy)-3,3-dimethyl-4-oxobutyl)-6,6-difluorohexahydropyrrolo[3,2-c]pyrazole-4(1H)-carboxylate S12-1 (70 mg, 0.15 mmol) in DCM (2 mL, 1.33 g/mL, 31.32 mmol) while stirring at 0° C. was added TFA (0.35 mL, 1.49 g/mL, 4.57 mmol). Then the mixture was stirred at 0° C. for 3 hrs. To the mixture was added 10 mL toluene and the mixture was concentrated under reduced pressure to give desired product (55 mg) as colorless oil, which was used in next step directly.

### Compound 70R: ethyl (4S)-6-((2-acetyl-1-(4-(tert-butoxy)-3,3-dimethyl-4-oxobutyl)-6,6-difluorohexahydropyrrolo[3,2-c]pyrazol-4(1H)-yl)methyl)-4-(3-fluoro-2-methylphenyl)-2-(thiazol-2-yl)-1,4-dihydropyrimidine-5-carboxylate

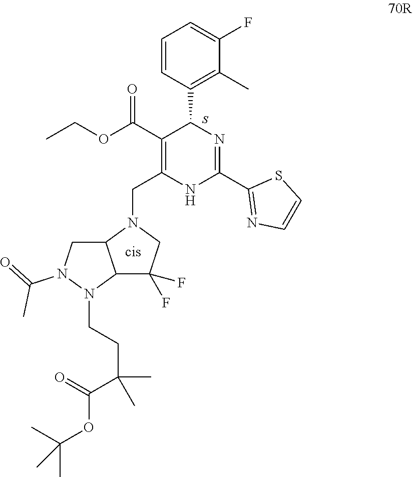

This compound was prepared from H2-1A and S12 according to typical coupling method 1. Purified by flash column chromatography on silica gel eluting with 0˜50% EtOAc in Hexanes. 100 mg of product was obtained as yellow oil.

### Compound 70: 4-(2-acetyl-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

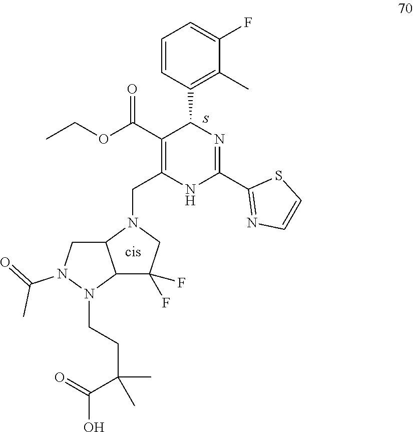

This compound was prepared from compound 70R using typical method 3 and purified by flash column chromatography (Column: C18, 20-35 μm, 100 Å, 40 g) eluting with 5-50% Acetonitrile in water (add 0.05% HCOOH). 9 mg was obtained as yellow solid. LC-MS (ESI): mass calcd. for C₃₁H₃₇F₃N₆O₅S 662.2, m/z found 663.3 [M+H]⁺. ¹H NMR (400 MHz, METHANOL-d4) δ ppm 7.85-7.92 (1H, m), 7.67-7.74 (1H, m), 7.03-7.24 (2H, m), 6.89-6.96 (1H, m), 5.94 (1H, d, J=7.58 Hz), 4.34-4.45 (1H, m), 3.88-4.34 (6H, m), 3.12-3.26 (2H, m), 2.73-3.08 (3H, m), 2.49 (3H, s), 2.19-2.37 (3H, m), 1.73-1.87 (2H, m), 1.16-1.26 (6H, m), 1.08-1.15 (3H, m).

### Compound 70A and 70B: 4-((cis)-2-Acetyl-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

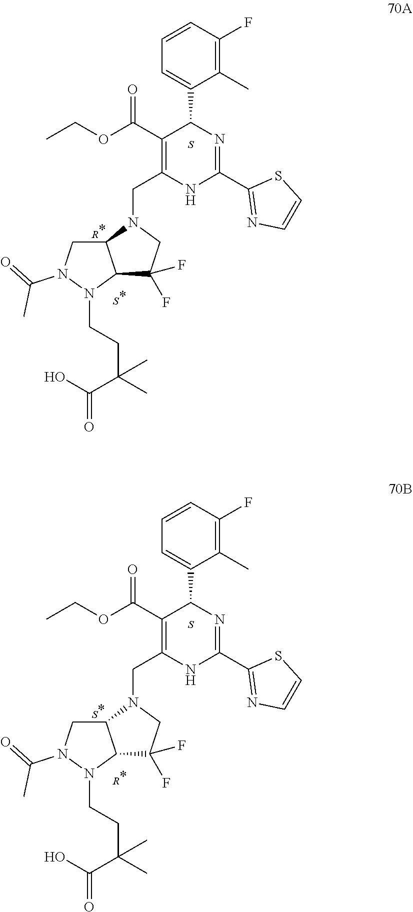

Compound 70A and 70B were made analogously.

Compound 70A: purified by prep-HPLC (Column: Waters Xbridge C18 (5 μm 19*150 mm), Mobile Phase A: water (0.1% trifluoroacetic acid), Mobile Phase B: acetonitrile, UV: 254 nm, Flow rate: 15 mL/min, Gradient: 30-75% (% B)) and further purified by by C18 (acetonitrile:water (0.1% ammonium bicarbonate)=20% to 70%) to give the title compound (22.5 mg, 95.2% purity, 39% yield) as yellow solids. LC-MS (ESI): mass calcd. for C₃₁H₃₇F₃N₆O₅S 662.3, m/z found 663.4 [M+H]⁺. ¹H NMR (400 MHz, CD₃OD) δ 7.89 (d, J=3.2 Hz, 1H), 7.71 (d, J=2.8 Hz, 1H), 7.17-7.15 (m, 2H), 6.97-6.92 (m, 1H), 5.98 (s, 1H), 4.45-4.37 (m, 2H), 4.11-4.05 (m, 4H), 4.01-3.94 (m, 1H), 3.26-3.22 (m, 1H), 3.19-3.14 (m, 1H), 2.96-2.86 (m, 3H), 2.51 (d, J=1.6 Hz, 3H), 2.35 (s, 3H), 1.87-1.81 (m, 2H), 1.24 (s, 3H), 1.23 (s, 3H), 1.14 (t, J=7.2 Hz, 3H).

Compound 70B: purified by prep-HPLC (Column: Waters Xbridge C18 (5 μm 19*150 mm), Mobile Phase A: water (0.1% ammonium bicarbonate), Mobile Phase B: acetonitrile, UV: 254 nm, Flow rate: 15 mL/min, Gradient: 25-70% (% B)) to give the title compound (39 mg, 97.9% purity, 53% yield) as yellow solids. LC-MS (ESI): mass calcd. for C₃₁H₃₇F₃N₆O₅S 662.3, m/z found 663.3 [M+H]⁺. ¹H NMR (400 MHz, CD₃OD) δ 7.79 (d, J=3.2 Hz, 1H), 7.59 (d, J=2.8 Hz, 1H), 7.07-6.98 (m, 2H), 6.83 (t, J=9.2 Hz, 1H), 5.84 (s, 1H), 4.23 (d, J=12.8 Hz, 1H), 4.11-4.03 (m, 3H), 3.98-3.86 (m, 3H), 3.15-3.08 (m, 2H), 2.95 (s, 1H), 2.83-2.67 (m, 2H), 2.39 (d, J=1.6 Hz, 3H), 2.16 (s, 3H), 1.75-1.66 (m, 2H), 1.12 (s, 3H), 1.11 (s, 3H), 1.02 (t, J=7.2 Hz, 3H).

### Preparation of INTERMEDIATE S35

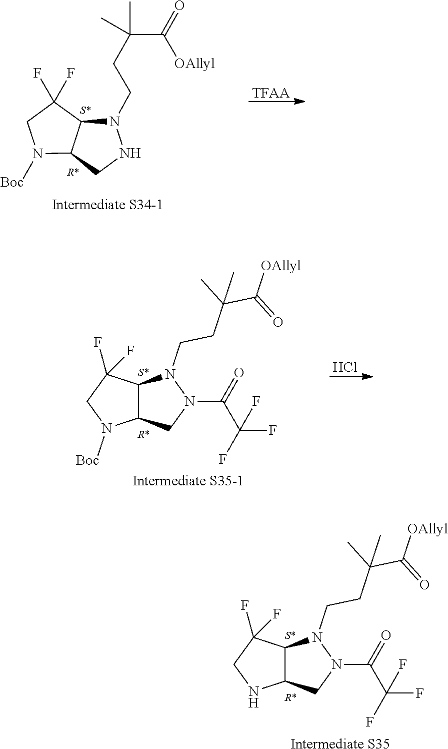

### S35-1: (cis)-tert-Butyl 1-(4-(allyloxy)-3,3-dimethyl-4-oxobutyl)-6,6-difluoro-2-(2,2,2-trifluoroacetyl)hexahydropyrrolo[3,2-c]pyrazole-4(2H)-carboxylate

To a solution of S34-1 (75 mg, 80% purity, 0.149 mmol) in dichloromethane (5 mL) were added triethylamine (75 mg, 0.741 mmol), N,N-dimethylpyridin-4-amine (7 mg, 0.057 mmol) and 2,2,2-trifluoroacetic anhydride (62 mg, 0.295 mmol) at room temperature. After heated at 25° C. overnight under nitrogen atmosphere, the reaction mixture was poured into saturated sodium bicarbonate aqueous solution (20 mL) and extracted with dichloromethane (20 mL) for three times. The combined organic layers were washed with brine (20 mL), dried over Na₂SO₄₍ₛ₎, filtered and concentrated under reduced pressure to give the title compound (70 mg, 90% purity from ¹H NMR, 85% yield) as colorless oil. LC-MS (ESI): mass calcd. for C₂₁H₃₀F₅N₃O₅ 499, m/z found 500.2 [M+H]⁺. ¹H NMR (400 MHz, CDCl₃) δ 5.96-5.86 (m, 1H), 5.35-5.24 (m, 2H), 4.76-4.45 (m, 4H), 3.92-3.74 (m, 2H), 3.56-3.36 (m, 2H), 2.99-2.79 (m, 2H), 1.89-1.74 (m, 2H), 1.49 (s, 3H), 1.45 (s, 6H), 1.25 (s, 3H), 1.20 (s, 3H).

### S35: (cis)-Allyl 4-(6,6-difluoro-2-(2,2,2-trifluoroacetyl)hexahydropyrrolo[3,2-c]pyrazol-1(2H)-yl)-2,2-dimethylbutanoate

A solution of S35-1 (70 mg, 90% purity, 0.126 mmol) in 4 M hydrochloride in ethyl acetate (3 mL) was stirred at room temperature for 1 hour. The reaction mixture was concentrated under reduced pressure. The residue was quenched with saturated sodium bicarbonate aqueous solution (20 mL) slowly and extracted with dichloromethane (20 mL) for three times. The combined organic layers were washed with brine (20 mL), dried over Na₂SO₄₍ₛ₎, filtered and concentrated under reduced pressure to give the title compound (55 mg, 90% purity from ¹H NMR, 98% yield) as colorless oil. LC-MS (ESI): mass calcd. for C₁₆H₂₂F₅N₃O₃ 399, m/z found 400.2 [M+H]⁺. ¹H NMR (300 MHz, CDCl₃) δ 5.98-5.85 (m, 1H), 5.35-5.23 (m, 2H), 4.57 (t, J=6.3 Hz, 2H), 4.34 (t, J=6.0 Hz, 1H), 4.27-4.19 (m, 1H), 3.77-3.62 (m, 2H), 3.49-3.43 (m, 1H), 3.20-3.09 (m, 1H), 2.94-2.76 (m, 2H), 1.92-1.75 (m, 2H), 1.24 (s, 3H), 1.20 (s, 3H).

### Compound 71: 4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluoro-2-(2,2,2-trifluoroacetyl)hexahydropyrrolo[3,2-c]pyrazol-1(2H)-yl)-2,2-dimethylbutanoic acid

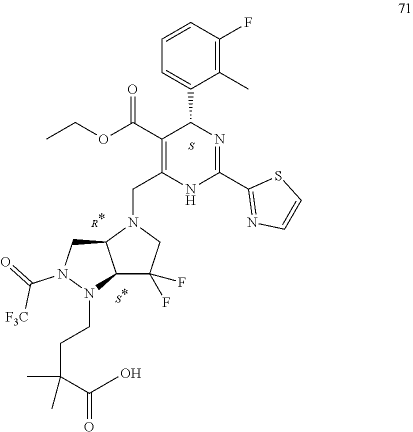

This compound was made from H12-1A and S35 according to typical coupling method 1 and typical method 2 successively. Purified by C18 column (acetonitrile:water=05% to 50%, then acetonitrile:water (0.1% ammonium bicarbonate)=50% to 95%) to give the title compound (35 mg, 97.0% purity, 66% yield) as yellow solids. LC-MS (ESI): mass calcd. for C₃₁H₃₄F₆N₆O₅S 716.2, m/z found 717.3 [M+H]⁺. ¹H NMR (400 MHz, DMSO-d₆) δ 12.11 (br s, 1H), 9.70 (br s, 0.3H), 9.33 (s, 0.1H), 9.28 (s, 0.6H), 8.03-8.01 (m, 0.6H), 7.91-7.86 (m, 0.8H), 7.82 (d, J=3.2 Hz, 0.6H), 7.21-7.16 (m, 1.3H), 7.09-7.02 (m, 1.7H), 5.89 (s, 0.7H), 5.77 (d, J=2.4 Hz, 0.3H), 4.32-4.15 (m, 3.7H), 4.11-3.95 (m, 3.3H), 3.81 (d, J=14.0 Hz, 0.3H), 3.56-3.40 (m, 1H), 3.20-3.13 (m, 1H), 3.06-2.84 (m, 1.7H), 2.74-2.66 (m, 1H), 2.44 (s, 2H), 2.41 (s, 1H), 1.76-1.68 (m, 1H), 1.64-1.54 (m, 1H), 1.11-1.02 (m, 9H).

### Preparation of Intermediates S34-A and S34-B

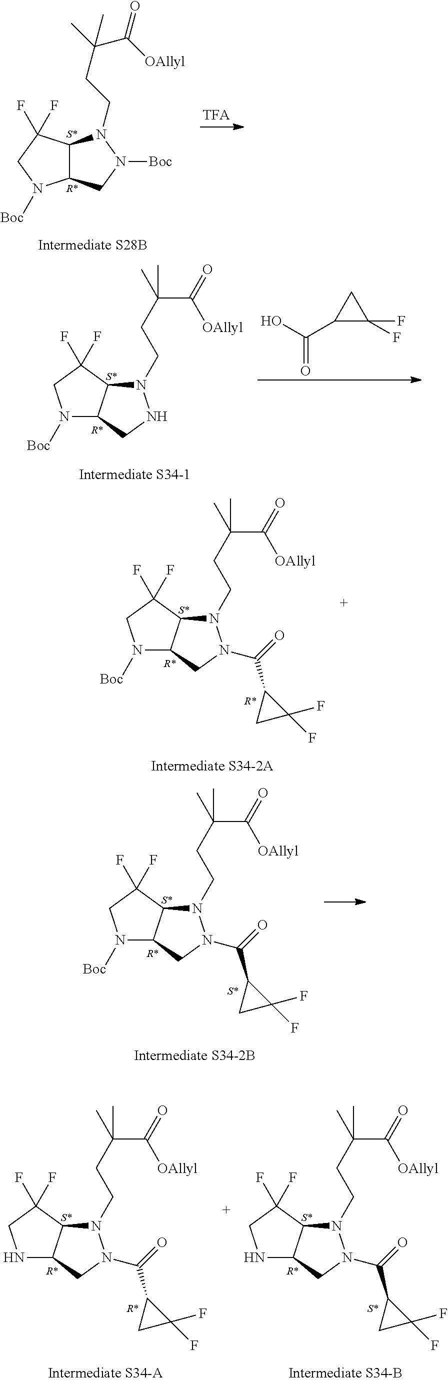

### S34-1: (cis)-tert-Butyl 1-(4-(allyloxy)-3,3-dimethyl-4-oxobutyl)-6,6-difluorohexahydropyrrolo[3,2-c]pyrazole-4(2H)-carboxylate

To a solution of S28B (400 mg, 90% purity, 0.715 mmol) in dichloromethane (10 mL) was added a mixture solution of trifluoroacetic acid (2 mL) and dichloromethane (30 mL) at 0° C. under nitrogen atmosphere. After stirred at 0° C. for 3 hours, the mixture was poured into saturated sodium bicarbonate solution (50 mL) and extracted with dichloromethane (50 mL) for three times. The combined organic layers were washed with brine (50 mL) then dried over Na₂SO₄₍ₛ₎ and concentrated to give the title compound (330 mg, 92% yield, 80% purity from ¹H NMR) as colorless oil. LC-MS (ESI): mass calcd. for C₁₉H₃₁F₂N₃O₄ 403.2, m/z found 404.2 [M+H]⁺. ¹H NMR (400 MHz, CDCl₃) δ 5.96-5.86 (m, 1H), 5.35-5.30 (m, 1H), 5.25-5.22 (m, 1H), 4.63-4.51 (m, 2H), 4.00-3.71 (m, 2H), 3.57-3.42 (m, 2H), 3.26-3.09 (m, 3H), 3.00-2.73 (m, 2H), 2.03-1.84 (m, 2H), 1.49-1.45 (m, 9H), 1.24-1.21 (m, 6H).

### S34-2A and S34-2B: (cis)-tert-Butyl 1-(4-(allyloxy)-3,3-dimethyl-4-oxobutyl)-2-(-2,2-difluorocyclopropanecarbonyl)-6,6-difluorohexahydropyrrolo[3,2-c]pyrazole-4(2H)-carboxylate

To a solution of S34-1 (330 mg, 80% purity, 0.654 mmol), 2,2-difluorocyclopropanecarboxylic acid (120 mg, 0.983 mmol) and N,N-diisopropylethylamine (253 mg, 1.958 mmol) in N,N-dimethylformamide (4 mL) was added 2-(7-aza-1H-benzotriazole-1-yl)-1;1;3;3-tetramethyluronium hexafluorophosphate (253 mg, 0.981 mmol) at room temperature under nitrogen atmosphere. After stirred at room temperature for 4 hours, the mixture was diluted with water (120 mL) and extracted with ethyl acetate (80 mL) for three times. The combined organic layers were washed with brine (100 mL) for three times, dried over Na₂SO₄₍ₛ₎ and filtered. The filtrate was concentrated and purified by silica gel column chromatography (petroleum ether:ethyl acetate=8:1 to 2:1) to give the title compound S34-2A (77 mg, 90% purity ¹H NMR, 21% yield) and compound S34-2B (155 mg, 90% purity ¹H NMR, 42% yield) as colorless oil.

S34-2A: LC-MS (ESI): R_{T}=2.514 min, mass calcd. for C₂₃H₃₃F₄N₃O₅ 507.2, m/z found 508.2 [M+H]⁺. ¹H NMR (400 MHz, CDCl₃) δ 5.96-5.86 (m, 1H), 5.34-5.29 (m, 1H), 5.27-5.23 (m, 1H), 4.83-4.75 (m, 0.5H), 4.70-4.64 (m, 0.5H), 4.57 (d, J=5.6 Hz, 2H), 4.49-4.44 (m, 1H), 3.95-3.70 (m, 2H), 3.49-3.25 (m, 3H), 2.83 (t, J=8.4 Hz, 2H), 2.20-2.12 (m, 1H), 1.92-1.78 (m, 2H), 1.70-1.61 (m, 1H), 1.48 (s, 4H), 1.45 (s, 5H), 1.24 (s, 3H), 1.22 (s, 3H).

S34-2B: LC-MS (ESI): R_{T}=2.499 min, mass calcd. for C₂₃H₃₃F₄N₃O₅ 507.2, m/z found 508.3 [M+H]⁺. ¹H NMR (400 MHz, CDCl₃) δ 5.99-5.83 (m, 1H), 5.34-5.30 (m, 1H), 5.27-5.25 (m, 1H), 4.76-4.47 (m, 4H), 4.03-3.68 (m, 2H), 3.49-3.26 (m, 2H), 3.20-3.12 (m, 1H), 2.92-2.73 (m, 2H), 2.22-2.09 (m, 1H), 1.91-1.79 (m, 2H), 1.76-1.66 (m, 1H), 1.48 (s, 3H), 1.44 (s, 6H), 1.26-1.23 (m, 6H).

### S34-A and S34-B: Allyl 4-((cis)-2-(2,2-difluorocyclopropanecarbonyl)-6,6-difluorohexahydropyrrolo[3,2-c]pyrazol-1(2H)-yl)-2,2-dimethylbutanoate

To a solution of S34-2A (70 mg, 90% purity, 0.124 mmol) in dichloromethane (1.5 mL) was added trifluoroacetic acid (1.5 mL) at room temperature under nitrogen atmosphere. After stirred at room temperature for 2 hours, the mixture was poured into saturated sodium bicarbonate solution (10 mL) and extracted with dichloromethane (10 mL) for three times. The combined organic layers were washed with brine (10 mL) and concentrated to give title compound (50 mg, 90% purity from ¹H NMR, 89% yield) as colorless oil. LC-MS (ESI): R_{T}=2.180 min, mass calcd. for C₁₈H₂₅F₄N₃O₃ 407.2, m/z found 408.2 [M+H]⁺. ¹H NMR (400 MHz, CDCl₃) δ 5.96-5.86 (m, 1H), 5.35-5.28 (m, 1H), 5.26-5.21 (m, 1H), 4.57 (d, J=5.6 Hz, 2H), 4.33 (t, J=6.4 Hz, 1H), 4.22 (d, J=12.8 Hz, 1H), 3.64-3.58 (m, 1H), 3.42-3.33 (m, 2H), 3.17-3.09 (m, 1H), 2.93-2.83 (m, 1H), 2.79 (t, J=16.0 Hz, 2H), 2.21-2.09 (m, 1H), 1.92-1.79 (m, 2H), 1.69-1.61 (m, 1H), 1.24 (s, 3H), 1.21 (s, 3H).

S34-B was made analogously. LC-MS (ESI): R_{T}=2.144 min, mass calcd. for C₁₈H₂₅F₄N₃O₃ 407.2, m/z found 408.2 [M+H]⁺. ¹H NMR (400 MHz, CDCl₃) δ 5.96-5.86 (m, 1H), 5.36-5.30 (m, 1H), 5.25 (dd, J=10.4, 1.2 Hz, 1H), 4.57 (dt, J=5.6, 1.2 Hz, 2H), 4.33 (t, J=6.4 Hz, 1H), 4.28 (t, J=12.8 Hz, 1H), 3.58 (dd, J=15.6, 6.8 Hz, 1H), 3.33 (dd, J=12.8, 6.4 Hz, 1H), 3.25-3.08 (m, 2H), 2.92-2.70 (m, 3H), 2.16-2.08 (m, 1H), 1.88-1.77 (m, 2H), 1.73-1.64 (m, 1H), 1.23 (s, 3H), 1.22 (s, 3H).

### Compound 72A: 4-((cis)-2-((R)-2,2-difluorocyclopropane-1-carbonyl)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

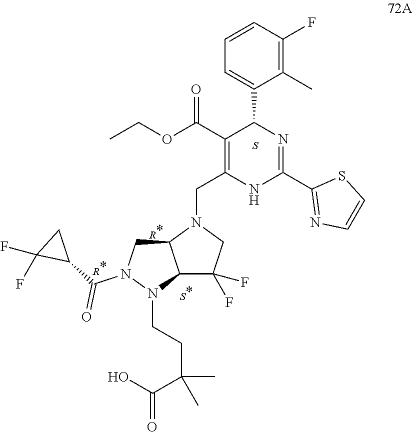

This compound was made from H12-1A and S34-A according to typical coupling method 1 and typical method 2 successively. Purified by C18 column (acetonitrile:water (+0.1% ammonium bicarbonateto)=5% to 100%) to give the title compound (40 mg, 96.0% purity, 56% yield) as yellow solids. LC-MS (ESI): mass calcd. for C₃₃H₃₇F₅N₆O₅S 724.2, m/z found 725.4 [M+H]⁺. ¹H NMR (400 MHz, CDCl₃) δ 8.92 (s, 1H), 7.74 (d, J=2.8 Hz, 1H), 7.39 (d, J=3.2 Hz, 1H), 7.12-7.06 (m, 1H), 7.01 (d, J=7.6 Hz, 1H), 6.90 (t, J=8.8 Hz, 1H), 5.99 (s, 1H), 4.50 (d, J=12.8 Hz, 1H), 4.39 (d, J=16.4 Hz, 1H), 4.10-4.00 (m, 4H), 3.86-3.79 (m, 1H), 3.58-3.50 (m, 1H), 3.25-3.21 (m, 1H), 3.16-3.09 (m, 1H), 2.92-2.81 (m, 3H), 2.52 (s, 1.5H), 2.51 (s, 1.5H), 2.18-2.10 (m, 1H), 1.90 (t, J=8.0 Hz, 2H), 1.68-1.58 (m, 1H), 1.28 (s, 3H), 1.25 (s, 3H), 1.11 (t, J=7.2 Hz, 3H).

### Compound 73: (cis)-4-(2-(cyclopropanecarbonyl)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

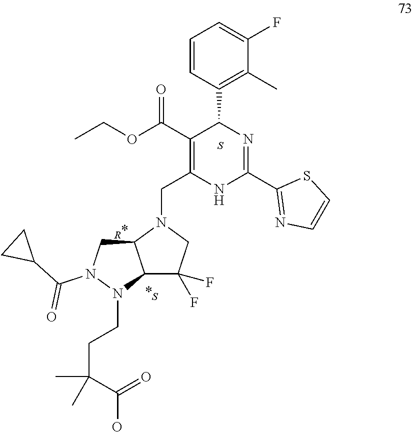

This compound was made from S34-1, Cyclopropanecarbonyl Chloride and H2-1A analogous to compound 71. LC-MS (ESI): mass calcd. for C₃₃H₃₉F₃N₆O₅S 688.3, found m/z 689.3 [M+H]⁺. ¹H NMR (400 MHz, METHANOL-d4) δ ppm 7.88 (1H, d, J=3.18 Hz), 7.70 (1H, d, J=3.06 Hz), 7.09-7.16 (2H, m), 6.88-6.97 (1H, m), 5.92-5.97 (1H, m), 4.28-4.39 (2H, m), 3.99-4.18 (5H, m), 3.22-3.29 (1H, m), 3.07-3.18 (1H, m), 2.83-3.03 (3H, m), 2.68-2.77 (1H, m), 2.44-2.54 (3H, m), 1.80-1.93 (2H, m), 1.20-1.23 (6H, m), 1.12 (3H, t, J=7.09 Hz), 0.96-1.03 (1H, m), 0.78-0.91 (2H, m), 0.69-0.77 (1H, m).

### Compound 74: 4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluoro-2-isobutyrylhexahydropyrrolo[3,2-c]pyrazol-1(2H)-yl)-2,2-dimethylbutanoic acid

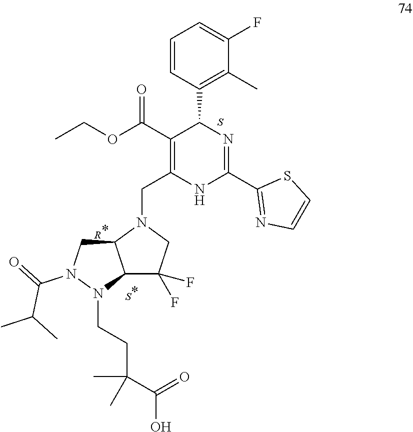

This compound was made from S34-1, Isobutyryl chloride and H2-1A analogous to compound 71. Purified by C18 Column: (acetonitrile:water (+0.1% ammonium bicarbonate)=20% to 75%) to give the title compound (20 mg, 94.7% purity, 56% yield) as yellow solids. LC-MS (ESI): mass calcd. for C₃₃H₄₁F₃N₆O₅S 690.2, m/z found 691.3 [M+H]⁺. ¹H NMR (400 MHz, CDCl₃) δ 8.98 (s, 1H), 7.80 (d, J=3.2 Hz, 1H), 7.37 (d, J=3.2 Hz, 1H), 7.09-7.04 (m, 1H), 6.96-6.88 (m, 2H), 5.99 (s, 1H), 4.50 (d, J=13.2 Hz, 1H), 4.23 (d, J=16.4 Hz, 1H), 4.12 (d, J=16.4 Hz, 1H), 4.08-3.98 (m, 3H), 3.86-3.79 (m, 1H), 3.20-3.09 (m, 2H), 3.00-2.92 (m, 1H), 2.87-2.74 (m, 2H), 2.53 (s, 3H), 1.95-1.88 (m, 1H), 1.82-1.75 (m, 1H), 1.25 (d, J=6.8 Hz, 3H), 1.15-1.11 (m, 9H).

### Compound 75: 4-((cis)-2-(2,2-Difluoroacetyl)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

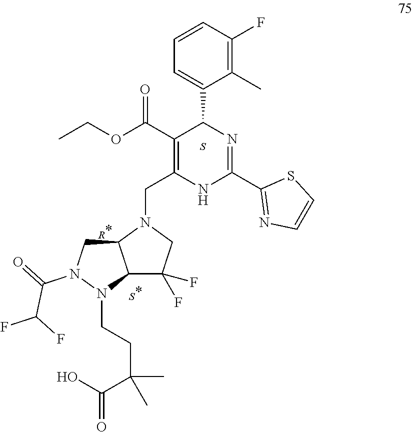

This compound was made from S34-1, difluoroacetic acid and H2-1A analogous to compound 71. LC-MS (ESI): mass calcd. for C₃₁H₃₅F₅N₆O₅S 698.2, m/z found 699.3 [M+H]⁺. ¹H NMR (400 MHz, DMSO-d₆) δ 8.02 (s, 0.7H), 7.89-7.85 (m, 1.3H), 7.20-7.17 (m, 1.3H), 7.08-7.02 (m, 1.7H), 6.87-6.56 (m, 1H), 5.89 (s, 0.7H), 5.78 (s, 0.3H), 4.33-4.22 (m, 2H), 4.16-4.08 (m, 3H), 4.00-3.95 (m, 2H), 3.35-3.31 (m, 1H), 3.14-3.09 (m, 1H), 3.03-2.79 (m, 2H), 2.70-2.63 (m, 1H), 2.44 (s, 2.1H), 2.41 (s, 0.9H), 1.72-1.58 (m, 2H), 1.15-1.03 (m, 9H)

### Preparation of Intermediate S78

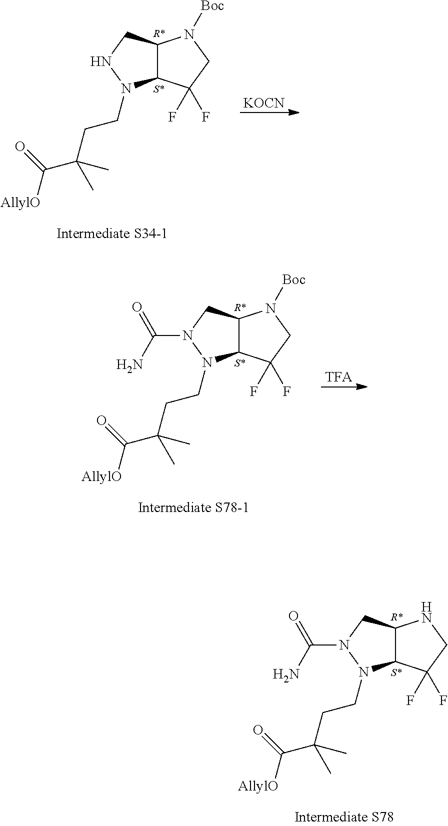

### S78-1: (cis)-tert-butyl 1-(4-(allyloxy)-3,3-dimethyl-4-oxobutyl)-2-carbamoyl-6,6-difluorohexahydropyrrolo[3,2-c]pyrazole-4(2H)-carboxylate

To a solution of S34-1 (90 mg, 0.178 mmol, 80% purity) in 1,4-dioxane (2 mL) was added water (2 mL), potassium cyanate (36 mg, 0.446 mmol) and acetic acid glycal (33 mg, 0.534 mmol) at room temperature. After stirred at 25° C. overnight, the mixture was diluted with water (10 mL) and extracted with dichloromethane (20 mL) twice. The combined organic phases were dried over Na₂SO₄₍ₛ₎, filtered and concentrated. The residue was purified by C18 column (acetonitrile:water=5% to 100%) to give the desired product (60 mg, 90% purity, 60% yield) as colorless oil. LC-MS (ESI): mass calcd. for C₂₀H₃₂F₂N₄O₅ 446.2, m/z found 447.3 [M+H]⁺. ¹H NMR (400 MHz, CDCl₃) δ 5.96-5.86 (m, 1H), 5.35-5.23 (m, 2H), 4.70-4.52 (m, 4H), 4.35-3.19 (m, 4H), 2.82-2.78 (m, 2H), 1.86-1.82 (m, 2H), 1.48-1.46 (m, 9H), 1.23-1.21 (m, 6H).

### S78: allyl 4-((cis)-2-carbamoyl-6,6-difluorohexahydropyrrolo[3,2-c]pyrazol-1(2H)-yl)-2,2-dimethylbutanoate

To a solution of S78-1 (60 mg, 0.134 mmol, 90% purity) in dichloromethane (10 mL) was added trifluoroacetic acid (2 mL) at room temperature. After stirred at room temperature for 1.5 hours, the mixture was quenched with saturated sodium bicarbonate aqueous solution (10 mL). It was extracted with dichloromethane (10 mL) twice and concentrated to give the title compound (49 mg, 19% purity, 20% yield) as yellow oil. LC-MS (ESI): mass calcd. for C₁₅H₂₄F₂N₄O₃ 346.2, m/z found 347.2 [M+H]⁺.

### Compound 76: 4-((cis)-2-carbamoyl-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

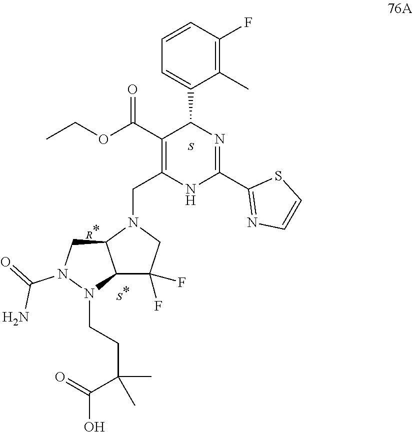

This compound was made from S78 and H2-1A according to typical method 1 and 2 successively. LC-MS (ESI): mass calcd. for C₃₀H₃₆F₃N₇O₅S 663.2, m/z found 664.3 [M+H]⁺. ¹H NMR (400 MHz, CD₃OD) δ 7.98 (d, J=3.2 Hz, 1H), 7.73 (s, 1H), 7.16 (s, 2H), 6.98-6.93 (m, 1H), 5.98 (s, 1H), 4.46-4.41 (m, 2H), 4.08 (q, J=7.2 Hz, 2H), 4.02-3.98 (m, 2H), 3.86-3.78 (m, 1H), 3.26-3.18 (m, 2H), 2.95-2.84 (m, 3H), 2.52 (s, 3H), 1.92-1.89 (m, 2H), 1.24 (s, 6H), 1.15 (t, J=7.2 Hz, 3H).

### Compound 77B: 4-((cis)-2-acetyl-4-((6-(2-chloro-3,4-difluorophenyl)-5-(ethoxycarbonyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

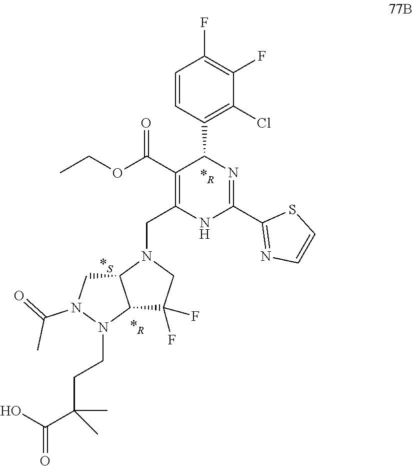

This compound was made analogous to compound 70 using H8-1A. purified by Prep. HPLC (Column: Xbrige (5 um 19*150 mm), Mobile Phase A: water (0.1% ammonium bicarbonate), Mobile Phase B: acetonitrile, UV: 254 nm, Flow rate: 15 mL/min, Gradient: 20-70% (% B)) to give the title compound (40 mg, 97.0% purity, 76% yield) as yellow solids. LC-MS (ESI): mass calcd. for C₃₀H₃₃ClF₄N₆O₅S 700.2, m/z found 701.2 [M+H]⁺. ¹H NMR (400 MHz, CDCl₃) δ 9.08 (s, 1H), 7.84 (d, J=3.2 Hz, 1H), 7.52 (s, 0.1H), 7.42 (d, J=2.8 Hz, 0.9H), 7.04-7.00 (m, 2H), 6.16 (s, 1H), 4.47 (d, J=13.2 Hz, 1H), 4.20 (d, J=16.8 Hz, 1H), 4.10-3.97 (m, 4H), 3.82-3.75 (m, 1H), 3.21-3.10 (m, 2H), 2.97-2.87 (m, 1H), 2.81 (t, J=8.4 Hz, 2H), 2.29 (s, 3H), 1.90-1.80 (m, 2H), 1.27 (s, 3H), 1.25 (s, 3H), 1.12 (t, J=7.2 Hz, 3H).

### Compound 79A: 4-((cis)-2-acetyl-4-((6-(2-chloro-3,4-difluorophenyl)-5-(methoxycarbonyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

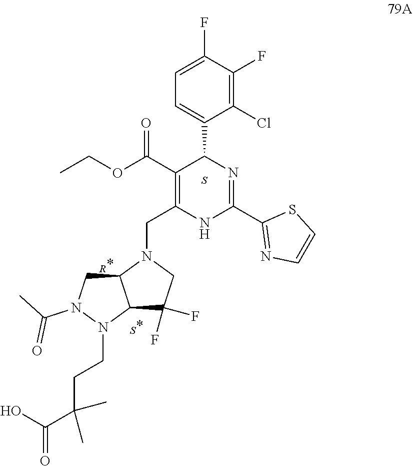

This compound was made analogous to compound 70 using H5-1A. Purified by C18 Column: (acetonitrile:water (+0.1% ammonium bicarbonate)=20% to 75%) to give the title compound (25 mg, 96.9% purity, 45% yield) as yellow solids. LC-MS (ESI): mass calcd. for C₂₉H₃₁ClF₄N₆O₅S 686.2, m/z found 687.2 [M+H]⁺. ¹H NMR (400 MHz, CDCl₃) δ 9.07 (s, 1H), 7.80 (d, J=3.2 Hz, 1H), 7.42 (d, J=3.2 Hz, 1H), 7.13-7.00 (m, 2H), 6.18 (s, 1H), 4.56 (d, J=12.8 Hz, 1H), 4.39 (d, J=17.2 Hz, 1H), 3.96-3.92 (m, 2H), 3.77-3.69 (m, 1H), 3.59 (s, 3H), 3.18-3.08 (m, 2H), 2.85-2.76 (m, 3H), 2.35 (s, 3H), 1.90-1.83 (m, 2H), 1.27 (s, 3H), 1.26 (s, 3H).

### Compound 79B: 4-((cis)-2-acetyl-4-((6-(2-chloro-3,4-difluorophenyl)-5-(methoxycarbonyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

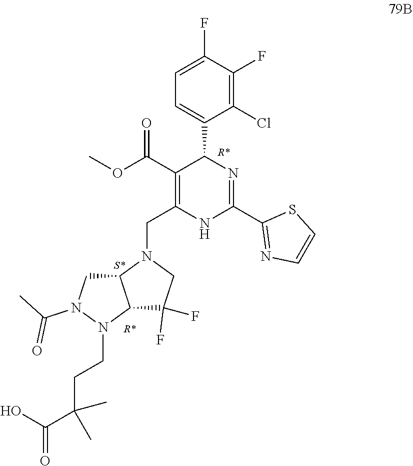 [SMILES: COC(=O)C1=C(CN2CC(F)(F)[C@H]3[C@@H]2CN(C(C)=O)N3CCC(C)(C)C(=O)O)NC(c2nccs2)=N[C@H]1c1ccc(F)c(F)c1Cl]

This compound was made analogous to compound 70 using H5-1A. Purified by C18 (acetonitrile:water=5% to 80%) to give the title compound (40 mg, 99.4% purity, 58% yield) as yellow solids. LC-MS (ESI): mass calcd. for C₂₉H₃₁ClF₄N₆O₅S 686.2, m/z found 687.2 [M+H]⁺. ¹H NMR (400 MHz, CDCl₃) δ 9.11 (br s, 1H), 7.84 (d, J=3.2 Hz, 1H), 7.43 (d, J=2.8 Hz, 1H), 7.07-7.00 (m, 2H), 6.14 (s, 1H), 4.48 (d, J=12.8 Hz, 1H), 4.18 (d, J=16.8 Hz, 1H), 4.11-3.98 (m, 2H), 3.81-3.74 (m, 1H), 3.59 (s, 3H), 3.21-3.11 (m, 2H), 3.02-2.87 (m, 1H), 2.81 (t, J=8.0 Hz, 2H), 2.29 (s, 3H), 1.91-1.77 (m, 2H), 1.26 (s, 3H), 1.25 (s, 3H).

### Compound 80B: 4-((cis)-2-acetyl-4-((6-(3,4-difluoro-2-methylphenyl)-5-(ethoxycarbonyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

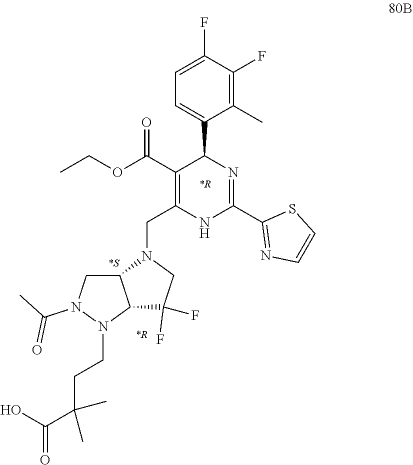 [SMILES: CCOC(=O)C1=C(CN2CC(F)(F)[C@H]3[C@@H]2CN(C(C)=O)N3CCC(C)(C)C(=O)O)NC(c2nccs2)=N[C@@H]1c1ccc(F)c(F)c1C]

This compound was made analogous to compound 70 using H9-1A. Purified by C18 column (acetonitrile:water(0.1% ammonium bicarbonate)=5% to 85%) to give the title compound (20 mg, 99.4% purity, 39% yield) as yellow solids. LC-MS (ESI): mass calcd. for C₃₁H₃₆F₄N₆O₅S 680.2, m/z found 681.3 [M+H]⁺. ¹H NMR (400 MHz, CDCl₃) δ 9.02 (s, 1H), 7.82 (d, J=2.8 Hz, 1H), 7.40 (d, J=3.2 Hz, 1H), 6.91-6.85 (m, 2H), 5.91 (s, 1H), 4.47 (d, J=13.2 Hz, 1H), 4.22 (d, J=16.8 Hz, 1H), 4.14-3.99 (m, 4H), 3.82-3.75 (m, 1H), 3.21-3.11 (m, 2H), 2.98-2.90 (m, 1H), 2.83-2.79 (m, 2H), 2.55 (d, J=2.4 Hz, 3H), 2.28 (s, 3H), 1.92-1.81 (m, 2H), 1.26 (s, 3H), 1.25 (s, 3H), 1.12 (t, J=7.2 Hz, 3H).

### Compound 81B: 4-((cis)-2-acetyl-4-((6-(2-chloro-3-fluorophenyl)-5-(ethoxycarbonyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

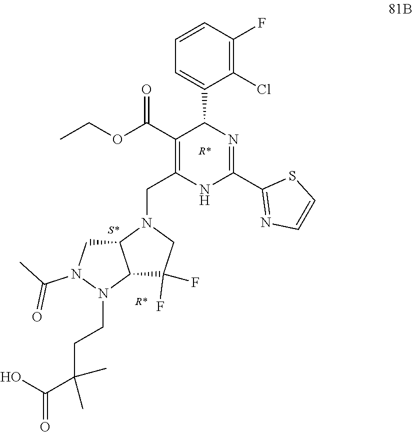 [SMILES: CCOC(=O)C1=C(CN2CC(F)(F)[C@H]3[C@@H]2CN(C(C)=O)N3CCC(C)(C)C(=O)O)NC(c2nccs2)=N[C@H]1c1cccc(F)c1Cl]

This compound was made analogous to compound 70 using H1-1A. Purified by C18 column (acetonitrile:water(0.1% Ammonium bicarbonate)=5% to 85%) to give the title compound (40 mg, 99.4% purity, 78% yield) as yellow solids. LC-MS (ESI): mass calcd. for C₃₀H₃₄ClF₃N₆O₅S 682.2, m/z found 683.2 [M+H]⁺. ¹H NMR (400 MHz, CDCl₃) δ 9.06 (s, 1H), 7.83 (d, J=2.8 Hz, 1H), 7.41 (d, J=3.2 Hz, 1H), 7.19-7.14 (m, 1H), 7.10 (d, J=7.6 Hz, 1H), 7.05-7.01 (m, 1H), 6.23 (s, 0.9H), 6.18 (s, 0.1H), 4.48 (d, J=13.2 Hz, 1H), 4.22 (d, J=16.8 Hz, 1H), 4.10-3.97 (m, 4H), 3.81-3.75 (m, 1H), 3.23-3.11 (m, 2H), 2.97-2.88 (m, 1H), 2.83-2.79 (m, 2H), 2.30 (s, 2.7H), 2.21 (s, 0.3H), 1.92-1.79 (m, 2H), 1.26 (s, 3H), 1.25 (s, 3H), 1.11 (t, J=7.2 Hz, 3H).

### Compound 82B: 4-((cis)-2-acetyl-4-((6-(3,4-difluoro-2-methylphenyl)-5-(methoxycarbonyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

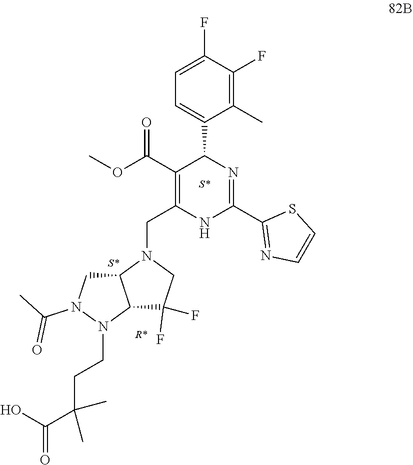 [SMILES: COC(=O)C1=C(CN2CC(F)(F)[C@H]3[C@@H]2CN(C(C)=O)N3CCC(C)(C)C(=O)O)NC(c2nccs2)=N[C@H]1c1ccc(F)c(F)c1C]

This compound was made analogous to compound 70 using H6-1B. Purified by prep. HPLC (preparation method: waters X-bridge C18 (5 μm 19*150 mm), Mobile Phase A: water (0.1% formic acid), Mobile Phase B: acetonitrile, UV: 214 nm, Flow rate: 15 mL/min, Gradient: 35-75% (% B)) to give the title compound (38 mg, 99.0% purity, 55.4% yield) as yellow solids. LC-MS (ESI): mass calcd. for C₃₀H₃₄F₄N₆O₅S 666.2, m/z found 667.2 [M+H]⁺. ¹H NMR (400 MHz, CDCl₃) δ 9.06 (s, 1H), 7.82 (d, J=2.8 Hz, 1H), 7.40 (d, J=3.2 Hz, 1H), 6.90-6.84 (m, 2H), 5.90 (s, 1H), 4.48 (d, J=13.2 Hz, 1H), 4.22 (d, J=17.2 Hz, 1H), 4.12 (d, J=16.8 Hz, 1H), 4.03 (t, J=6.4 Hz, 1H), 3.82-3.77 (m, 1H), 3.59 (s, 3H), 3.18-3.11 (m, 2H), 2.97-2.87 (m, 1H), 2.81 (t, J=8.0 Hz, 2H), 2.56 (d, J=2.0 Hz, 3H), 2.29 (s, 3H), 1.88-1.80 (m, 2H), 1.26 (s, 3H), 1.25 (s, 3H).

### Compound 83A: 4-((cis)-2-acetyl-4-((5-(ethoxycarbonyl)-2-(thiazol-2-yl)-6-(2,3,4-trifluorophenyl)-3,6-dihydropyrimidin-4-yl)methyl)-6,6-difluorohexahydropyrrolo[3,2-c]pyrazol-1(2H)-yl)-2,2-dimethylbutanoic acid

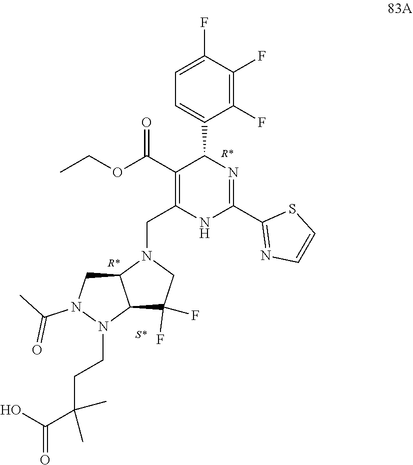 [SMILES: CCOC(=O)C1=C(CN2CC(F)(F)[C@@H]3[C@H]2CN(C(C)=O)N3CCC(C)(C)C(=O)O)NC(c2nccs2)=N[C@H]1c1ccc(F)c(F)c1F]

This compound was made analogous to compound 70 using H25-1A. Purified by Prep. HPLC (Column: sunfire waters C18 (5 um 19*150 mm), Mobile Phase A: Water (0.1% trifluoroacetic acid), Mobile Phase B: acetonitrile, UV: 214 nm, Flow rate: 15 mL/min, Gradient: 30-70% (% B)) to give the desired product, which was further purified by C18 column (acetonitrile:water (0.1% ammonium bicarbonate)=05% to 95%) to give the title compound (21 mg, 99.2% purity, 49% yield) as yellow solids. LC-MS (ESI): mass calcd. for C₃₀H₃₃F₅N₆O₅S 684.2, m/z found 685.2 [M+H]⁺. ¹H NMR (400 MHz, DMSO-d₆) δ 8.06-8.01 (m, 0.4H), 7.94 (s, 1.6H), 7.31-7.19 (m, 2H), 5.94 (s, 0.9H), 5.84 (s, 0.1H), 4.25-4.19 (m, 2H), 4.07-3.92 (m, 5H), 3.05-2.93 (m, 3.5H), 2.81-2.67 (m, 1.5H), 2.12 (s, 2.5H), 2.05-2.02 (m, 0.5H), 1.66 (t, J=8.4 Hz, 2H), 1.09-1.07 (m, 9H)

### Compound 83B: 4-((cis)-2-acetyl-4-((5-(ethoxycarbonyl)-2-(thiazol-2-yl)-6-(2,3,4-trifluorophenyl)-3,6-dihydropyrimidin-4-yl)methyl)-6,6-difluorohexahydropyrrolo[3,2-c]pyrazol-1(2H)-yl)-2,2-dimethylbutanoic acid

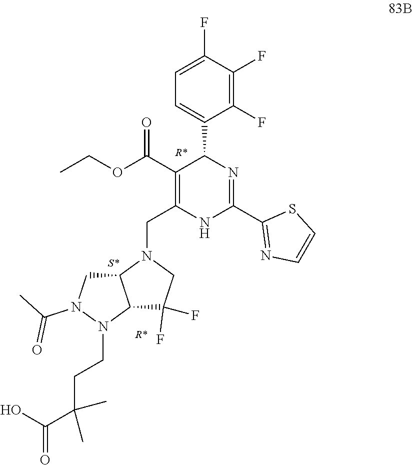 [SMILES: CCOC(=O)C1=C(CN2CC(F)(F)[C@H]3[C@@H]2CN(C(C)=O)N3CCC(C)(C)C(=O)O)NC(c2nccs2)=N[C@H]1c1ccc(F)c(F)c1F]

This compound was made analogous to compound 70 using H25-1A. Purified by Prep. HPLC (Column: sunfire waters C18 (5 um 19*150 mm), Mobile Phase A: Water (0.1% trifluoroacetic acid), Mobile Phase B: acetonitrile, UV: 214 nm, Flow rate: 15 mL/min, Gradient: 20-70% (% B)) to give the desired product, which was further purified by C18 column (acetonitrile:water (0.1% ammonium bicarbonate)=05% to 95%) to give the title compound (30 mg, 99.4% purity, 70% yield) as yellow solids. LC-MS (ESI): mass calcd. for C₃₀H₃₃F₅N₆O₅S 684.2, m/z found 685.2 [M+H]⁺. ¹H NMR (400 MHz, CDCl₃) δ 9.15 (br s, 1H), 7.86 (d, J=3.2 Hz, 1H), 7.43 (d, J=2.8 Hz, 1H), 7.05-6.99 (m, 1H), 6.92-6.86 (m, 1H), 5.97 (s, 1H), 4.46 (d, J=12.8 Hz, 1H), 4.17 (d, J=16.8 Hz, 1H), 4.09-4.04 (m, 2.7H), 4.00-3.94 (m, 1.3H), 3.80-3.73 (m, 1H), 3.22-3.09 (m, 2H), 2.91-2.79 (m, 3H), 2.31 (s, 3H), 1.88-1.79 (m, 2H), 1.25 (s, 3H), 1.24 (s, 3H), 1.17 (t, J=7.2 Hz, 3H).

### Compound 86: 4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluoro-2-formylhexahydropyrrolo[3,2-c]pyrazol-1(2H)-yl)-2,2-dimethylbutanoic acid

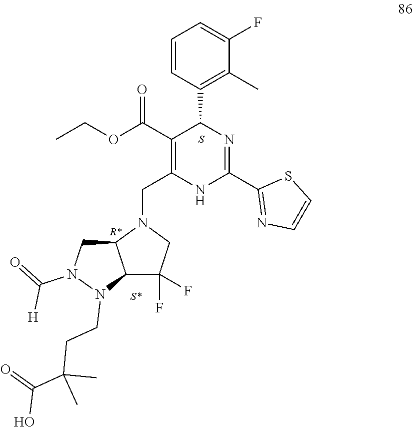

This compound was made from S28B and H2-1A analogous to compound 75. LC-MS (ESI): mass calcd. for C₃₀H₃₅F₃N₆O₅S 648.3, m/z found 649.3 [M+H]⁺. ¹H NMR (400 MHz, CDCl₃) δ 8.96 (s, 1H), 8.47 (d, J=2.4 Hz, 1H), 7.82 (d, J=3.2 Hz, 1H), 7.39 (d, J=3.2 Hz, 1H), 7.11-7.06 (m, 1H), 7.02-7.00 (m, 1H), 6.92-6.88 (m, 1H), 6.00 (s, 1H), 4.43 (d, J=16.4 Hz, 1H), 4.32 (d, J=12.8 Hz, 1H) 4.10-3.96 (m, 4H), 3.77-3.70 (m, 1H), 3.16-3.09 (m, 2H), 2.89-2.74 (m, 3H), 2.52 (s, 3H), 1.86-1.82 (m, 2H), 1.26 (s, 3H), 1.24 (s, 3H), 1.11 (t, J=7.2 Hz, 3H).

### Preparation of Intermediate S59

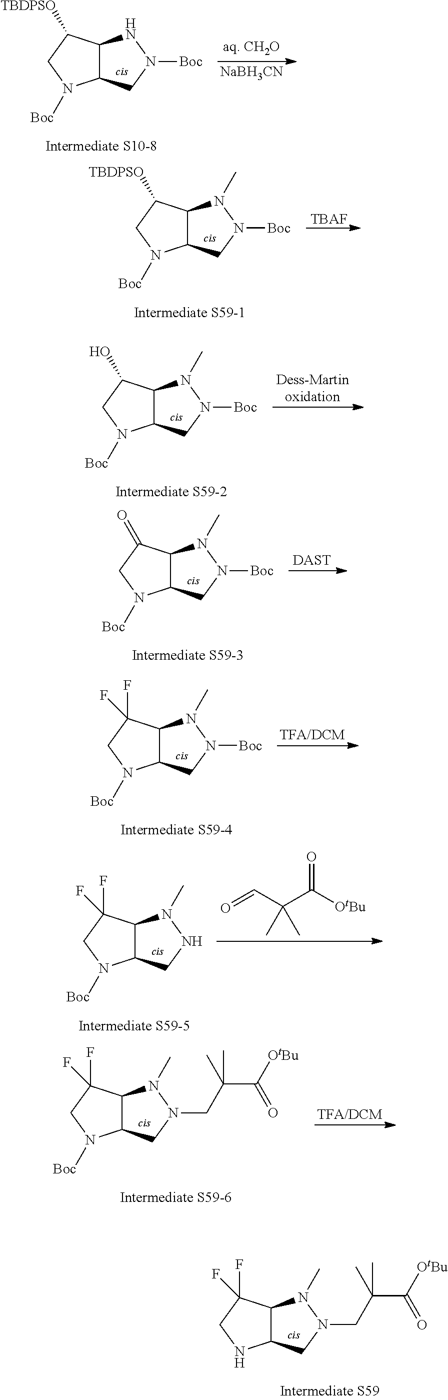

### S59-1: (cis)-di-tert-Butyl 6-((tert-butyldiphenylsilyl)oxy)-1-methylhexahydropyrrolo[3,2-c]pyrazole-2,4-dicarboxylate

To a solution of S10-8 (3.0 g, 90% purity, 4.76 mmol) in methanol (50 mL) and acetic acid (5 mL) was added 37% of formaldehyde aqueous solution (6.5 mL, 87.3 mmol). After the mixture was stirred at room temperature for 30 mins, sodium cyanoborohydride (650 mg, 10.3 mmol) was added by portion wise. The reaction mixture was stirred at 30° C. for 16 hours and quenched by water (50 mL). The mixture was extracted with ethyl acetate (50 mL) twice. The combined organic layers were washed by saturated sodium bicarbonate aqueous solution (100 mL) and brine (100 mL). The organic layer was dried over Na₂SO₄₍ₛ₎, filtered and concentrated under reduced pressure to give the title compound (2.80 g, 96% purity, 97% yield) as white solids. LC-MS (ESI): mass calcd. for C₃₂H₄₇N₃O₅Si 581.8, m/z found 582.4 [M+H]⁺.

### S59-2: (cis)-di-tert-Butyl 6-hydroxy-1-methylhexahydropyrrolo[3,2-c]pyrazole-2,4-dicarboxylate

To a solution of S59-1 (2.8 g, 96% purity, 4.62 mmol) in tetrahydrofuran (20 mL) was added 1 M tetrabutylammonium fluoride in tetrahydrofuran (10 mL, 10.0 mmol) at room temperature. After stirred at room temperature for 4 hours, the mixture was concentrated under reduced pressure to give a residue, which was purified by silica gel column chromatography (petroleum ether:ethyl acetate=1:1 to 1:4) to give the title compound (1.3 g, 90% purity from ¹H NMR, 74% yield) as colorless oil. ¹H NMR (400 MHz, CDCl₃) δ 4.56-4.46 (m, 1H), 4.29-4.22 (m, 1H), 4.12-3.98 (m, 1H), 3.51-3.30 (m, 4H), 2.62 (s, 3H), 2.40-2.34 (m, 1H), 1.49-1.44 (m, 18H).

### S59-3: (cis)-di-tert-Butyl 1-methyl-6-oxohexahydropyrrolo[3,2-c]pyrazole-2,4-dicarboxylate

To a solution of S59-2 (2.6 g, 90% purity, 6.81 mmol) in dichloromethane (50 mL) was added Dess-Martin periodinane (11.7 g, 27.6 mmol) at 0° C. under nitrogen atmosphere. After the mixture was stirred at room temperature for 16 hours, saturated sodium bicarbonate solution (100 mL) was added. The reaction mixture was extracted with dichloromethane (50 mL) for three times. The combined organic layers were washed with brine (100 mL), dried over Na₂SO₄₍ₛ₎ and filtered. The filtrate was concentrated under reduced pressure to give a residue, which was purified by silica gel column chromatography (petroleum ether:ethyl acetate=2:1 to 1:1) to give the title compound (1.5 g, 80% purity from ¹H NMR, 52% yield) as white solids. ¹H NMR (400 MHz, CDCl₃) δ 4.84-4.75 (m, 1H), 4.34-4.29 (m, 0.6H), 4.22-4.19 (m, 0.4H), 3.82-3.75 (m, 2H), 3.53-3.42 (m, 2H), 2.69 (s, 3H), 1.48-1.45 (m, 18H).

### S59-4: (cis)-di-tert-Butyl 6,6-difluoro-1-methylhexahydropyrrolo[3,2-c]pyrazole-2,4-dicarboxylate

A solution of S59-3 (1.5 g, 80% purity, 3.52 mmol) in dichloromethane (50 mL) at −78° C. was added diethylaminosulfur trifluoride (3.0 mL, 22.7 mmol). After stirred at room temperature for 3 hours, the reaction mixture was added to the saturated aqueous sodium bicarbonate (100 mL). The two layers were separated and the aqueous layer was extracted with dichloromethane (50 mL). The combined organic layers were washed with brine (100 mL), dried over Na₂SO₄₍ₛ₎, filtered and concentrated under reduced pressure to give a residue, which was purified by silica gel column chromatography (petroleum ether:ethyl acetate=30:1 to 10:1) to give the title product (850 mg, 95% purity from ¹H NMR, 63% yield) as yellowish solids. ¹H NMR (400 MHz, CDCl₃) δ 4.57-4.47 (m, 1H), 4.37-4.23 (m, 1H), 3.88-3.69 (m, 1H), 3.56-3.47 (m, 1H), 3.37-3.30 (m, 2H), 2.71 (d, J=4.0 Hz, 3H), 1.49-1.45 (m, 18H).

### S59-5: (cis)-tert-Butyl 6,6-difluoro-1-methylhexahydropyrrolo[3,2-c]pyrazole-4(2H)-carboxylate

To the solution of S59-4 (350 mg, 95% purity, 0.915 mmol) in dichloromethane (33.3 mL) was added trifluoroacetic acid (1.7 mL) at 0° C. under nitrogen atmosphere. After stirred at 0° C. for 3 hours, the reaction mixture was added to the saturated aqueous sodium bicarbonate solution (100 mL). The two layers were separated and the aqueous phase was extracted with dichloromethane (30 mL) twice. The combined organic extracts were washed with brine (100 mL), dried over Na₂SO₄₍ₛ₎, filtered and concentrated under reduced pressure to give the crude product (230 mg, 80% purity from ¹H NMR, 75% yield) as yellowish solids. ¹H NMR (400 MHz, CDCl₃) δ 4.66-4.57 (m, 1H), 3.99-3.80 (m, 1H), 3.64-3.53 (m, 1H), 3.21-3.08 (m, 3H), 2.65 (s, 3H), 1.47 (s, 9H).

### S59-6: (cis)-tert-Butyl 2-(3-(tert-butoxy)-2,2-dimethyl-3-oxopropyl)-6,6-difluoro-1-methylhexahydropyrrolo[3,2-c]pyrazole-4(2H)-carboxylate

To a mixture of S59-5 (230 mg, 80% purity, 0.699 mmol) in 1,2-dichloroethane (10 mL) was added tert-butyl 2,2-dimethyl-3-oxopropanoate (400 mg, 70% purity, 1.63 mmol). After the mixture was refluxed for 3 hours, sodium triacetoxyborohydride (700 mg, 3.30 mmol) was added by portion wise. Then the reaction mixture was stirred at room temperature overnight.

The mixture was diluted with water (50 mL) and extracted with dichloromethane (30 mL) for three times. The combined organic layers were dried over Na₂SO₄₍ₛ₎ and filtered. The filtrate was concentrated under reduced pressure to give crude, which was purified by silica gel column chromatography (petroleum ether:ethyl acetate=15:1 to 5:1) to give the title compound (210 mg, 90% purity from ¹H NMR, 64% of yield) as colourless oil. ¹H NMR (400 MHz, CDCl₃) δ 4.56-4.47 (m, 1H), 3.95-3.88 (m, 0.5H), 3.81-3.74 (m, 1.5H), 3.37-3.31 (m, 1H), 3.19-3.13 (m, 1.5H), 3.08-3.05 (m, 1H), 2.98-2.95 (m, 0.5H), 2.74-2.69 (m, 1H), 2.54 (d, J=3.6 Hz, 3H), 1.48-1.44 (m, 18H), 1.12-1.10 (m, 6H).

### S59: tert-Butyl 3-((cis)-6,6-difluoro-1-methylhexahydropyrrolo[3,2-c]pyrazol-2(1H)-yl)-2,2-dimethylpropanoate

To the solution of S59-6 (100 mg, 90% purity, 0.215 mmol) in dichloromethane (9.5 mL) was added trifluoroacetic acid (0.5 mL) at 0° C. under nitrogen atmosphere. After stirred at 0° C. for 3 hours, the reaction mixture was added to the saturated aqueous sodium bicarbonate solution (50 mL). The two layers were separated and the aqueous layer was extracted with dichloromethane (20 mL) twice. The combined organic extracts were washed with brine (50 mL), dried over Na₂SO₄₍ₛ₎ and filtered. The filtrate was concentrated under reduced pressure to give the crude product (65 mg, 90% purity from ¹H NMR, 85% yield) as yellowish solids. ¹H NMR (400 MHz, CDCl₃) δ 4.15-4.10 (m, 1H), 3.46-3.41 (m, 1H), 3.31-3.21 (m, 1H), 3.10-3.01 (m, 2H), 2.97-2.91 (m, 1H), 2.63 (d, J=12.8 Hz, 1H), 2.50 (s, 3H), 2.44-2.40 (m, 1H), 1.44 (s, 9H), 1.14 (s, 6H).

### Compound 87: 3-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluoro-1-methylhexahydropyrrolo[3,2-c]pyrazol-2(1H)-yl)-2,2-dimethylpropanoic acid

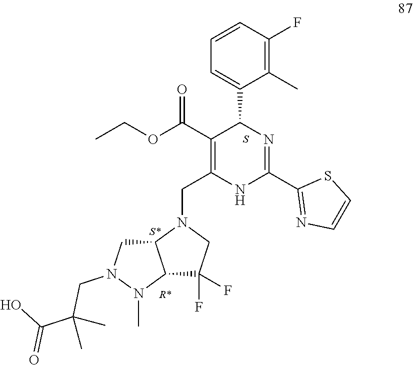

This compound was made from H2-1A and S59 according to typical method 1 and 3 successively. Chiral separation of tert-Butyl ester intermediate compounds: chiral prep.HPLC (Column: Chiralpak IG 5 μm 20*250 mm; Mobile Phase: Hex:EtOH=90:10 at 15 mL/min; Temp: 30° C.; Wavelength: 230 nm).

Compound 87, LC-MS (ESI): mass calcd. for C₂₉H₃₅F₃N₆O₄S 620.6, m/z found 621.3 [M+H]⁺. ¹H NMR (400 MHz, CD₃OD) δ 7.89 (d, J=2.8 Hz, 1H), 7.72 (d, J=3.2 Hz, 1H), 7.17-7.14 (m, 2H), 6.97-6.93 (m, 1H), 6.00 (s, 1H), 4.26 (d, J=16.8 Hz, 1H), 4.15 (d, J=16.4 Hz, 1H), 4.07 (q, J=7.2 Hz, 3H), 4.01-3.98 (m, 1H), 3.64-3.58 (m, 1H), 3.49-3.36 (m, 2H), 3.17-3.06 (m, 2H), 2.90-2.85 (m, 1H), 2.58 (s, 3H), 2.52 (d, J=1.6 Hz, 3H), 1.21 (s, 3H), 1.15-1.12 (m, 6H).

### Compound 89: 4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluoro-2-isopropylhexahydropyrrolo[3,2-c]pyrazol-1(2H)-yl)-2,2-dimethylbutanoic acid

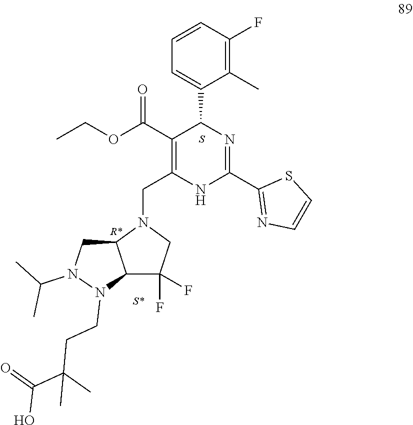

This compound was made from S28B, acetone and H2-1A analogous to Compound 90. LC-MS (ESI): mass calcd. for C₃₂H₄₁F₃N₆O₄S 662.3, m/z found 663.3. ¹H NMR (400 MHz, CD₃OD) δ 7.86 (d, J=3.2 Hz, 1H), 7.71 (d, J=3.2 Hz, 1H), 7.14-7.08 (m, 2H), 6.95-6.91 (m, 1H), 5.98 (s, 1H), 4.28 (d, J=16.4 Hz, 1H), 4.10-4.02 (m, 3H), 3.97-3.93 (m, 1H), 3.82-3.73 (m, 1H), 3.68-3.61 (m, 1H), 3.46 (d, J=13.2 Hz, 1H), 3.37-3.32 (m, 1H), 3.04-2.98 (m, 1H), 2.87-2.80 (m, 2H), 2.76-2.69 (m, 1H), 2.50 (s, 3H), 1.88-1.83 (m, 1H), 1.75-1.67 (m, 1H), 1.23 (d, J=6.0 Hz, 3H), 1.19 (s, 6H), 1.11 (t, J=7.2 Hz, 3H), 0.99 (d, J=6.0 Hz, 3H).

### Preparation of Intermediate S28

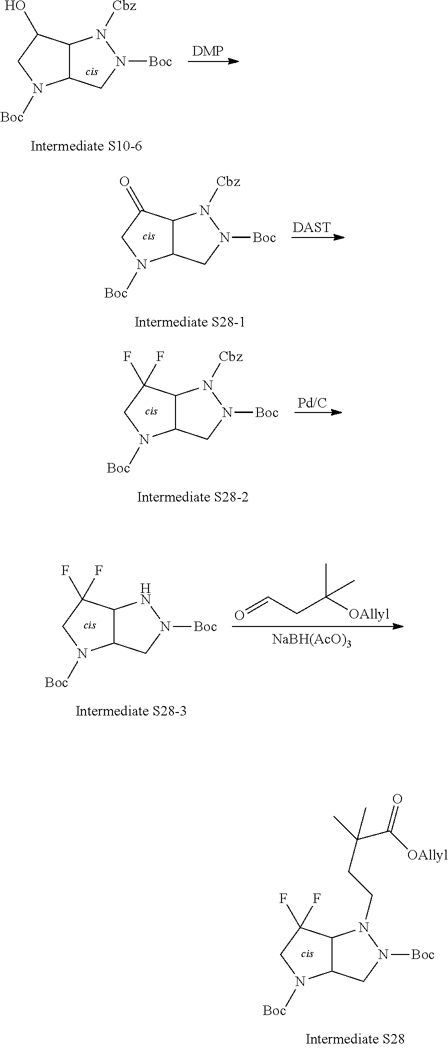

### S28-1: (cis)-1-Benzyl 2,4-di-tert-butyl 6-oxotetrahydropyrrolo[3,2-c]pyrazole-1,2,4(5H)-tricarboxylate

To a solution of S10-6 (5.86 g, 90% purity, 11.4 mmol) in dichloromethane (235 mL) was added Dess-Martin periodinane (8.60 g, 20.3 mmol) at room temperature under nitrogen atmosphere. After stirred at room temperature for 5 hours, saturated sodium bicarbonate solution (100 mL) was added and the reaction mixture was extracted with dichloromethane (50 mL) for three times. The combined organic layers were washed with brine (50 mL) then dried over Na₂SO₄₍ₛ₎, filtered and concentrated to give a residue, which was purified by silica gel column chromatography (petroleum ether:ethyl acetate=8:1 to 2:1) to give the title compound (5.76 g, 99% yield, 90% purity from ¹H NMR) as white solids. LC-MS (ESI): mass calcd. for C₂₃H₃₁N₃O₇ 461.2, m/z found 497.3 [M+NH₄]⁺. ¹H NMR (400 MHz, CDCl₃) δ 7.40-7.29 (m, 5H), 5.29 (d, J=12.4 Hz, 1H), 5.17 (d, J=12.4, 1H), 4.74-4.66 (m, 2H), 4.56-4.39 (m, 1H), 3.88-3.71 (m, 2H), 3.22-3.18 (m, 1H), 1.52-1.41 (m, 18H).

### S28-2: (cis)-1-Benzyl 2,4-di-tert-butyl 6,6-difluorotetrahydropyrrolo[3,2-c]pyrazole-1,2,4(5H)-tricarboxylate

To a solution of (cis)-1-benzyl 2,4-di-tert-butyl 6-oxotetrahydropyrrolo[3,2-c]pyrazole-1,2,4(5H)-tricarboxylate S28-1 (5.76 g, 90% purity, 11.2 mmol) in dichloromethane (125 mL) was added dropwise a solution of diethylaminosulfur trifluoride (27.5 g, 171 mmol) in dichloromethane (25 mL) at −78° C. under nitrogen atmosphere. After stirred at 40° C. for 48 hours, the mixture was slowly added into saturated sodium bicarbonate aqueous solution until to pH 8˜9 and extracted with dichloromethane (80 mL) for three times. The combined organic layers were dried over Na₂SO₄₍ₛ₎ and filtered. The filtrate was concentrated and purified by silica gel column chromatography (petroleum ether:ethyl acetate=10:1 to 4:1) to give the title compound (4.62 g, 90% purity from ¹H NMR, 77% yield) as light yellow solids. LC-MS (ESI): mass calcd. for C₂₃H₃₁F₂N₃O₆ 483.2, m/z found 327.9 [M−112+H]⁺. ¹H NMR (400 MHz, CDCl₃) δ 7.38-7.30 (m, 5H), 5.29 (d, J=12.0 Hz, 1H), 5.17 (d, J=12.0 Hz, 1H), 4.72-4.61 (m, 1.6H), 4.60-4.54 (m, 0.4H), 4.51-4.21 (m, 1H), 3.99-3.91 (m, 0.4H), 3.86-3.79 (m, 0.6H), 3.56-3.38 (m, 1H), 3.17-3.14 (m, 1H), 1.50-1.43 (m, 18H).

### S28-3: (cis)-Di-tert-butyl 6,6-difluorohexahydropyrrolo[3,2-c]pyrazole-2,4-dicarboxylate

To a solution of (cis)-1-benzyl 2,4-di-tert-butyl 6,6-difluorotetrahydropyrrolo[3,2-c]pyrazole-1,2,4(5H)-tricarboxylate S28-2 (4.62 g, 90% purity, 8.60 mmol) in ethanol (100 mL) were added one drop of 28% ammonium hydroxide solution and 10% palladium on charcoal wt. (3.64 g, 3.42 mmol) at room temperature. After stirred at room temperature under hydrogen atmosphere of balloon for 2 hours, 10% palladium on charcoal wt (1.0 g, 0.940 mmol) was added into the mixture and stirring continued for another 1 hour. The mixture was filtered and the filtrate was concentrated under reduced pressure to give the title compound (3.27 g, 90% purity from ¹H NMR, 98% yield) as white solids. LC-MS (ESI): mass calcd. for C₁₅H₂₅F₂N₃O₄ 349.2, m/z found 194.1 [M−156+H]⁺. ¹H NMR (400 MHz, CDCl₃) δ 4.65-4.62 (m, 0.7H), 4.59-4.48 (m, 1.3H), 4.30-4.27 (m, 0.6H), 4.17-4.14 (m, 0.4H), 3.98-3.88 (m, 1.4H), 3.84-3.76 (m, 0.6H), 3.56-3.45 (m, 1H), 3.32-3.21 (m, 1H), 1.50-1.46 (m, 18H).

### S28: (cis)-Di-tert-butyl 1-(4-(allyloxy)-3,3-dimethyl-4-oxobutyl)-6,6-difluorohexahydropyrrolo[3,2-c]pyrazole-2,4-dicarboxylate

To a solution of (cis)-di-tert-butyl 6,6-difluorohexahydropyrrolo[3,2-c]pyrazole-2,4-dicarboxylate S28-3 (1.00 g, 90% purity, 2.58 mmol), allyl 2,2-dimethyl-4-oxobutanoate (975 mg, 90% purity, 5.16 mmol) and 4A molecular sieves (2.0 g) in 1,2-dichloroethane (18 mL) was added acetic acid glacial (1.8 mL) under nitrogen atmosphere at room temperature. After stirred at 80° C. for 3 hours, sodium triacetoxyborohydride (2.73 g, 12.9 mmol) was added at room temperature. After stirred at room temperature for 1 hour, the mixture was quenched with ice water (20 mL), basified with saturated sodium bicarbonate aqueous solution to pH 8˜9 and extracted with dichloromethane (30 mL) for three times, The combined organic layers were washed with brine (20 mL), dried over Na₂SO₄₍ₛ₎ and filtered. The filtrate was concentrated to give a residue, which was purified by silica gel column chromatography (petroleum ether:ethyl acetate=13:1 to 10:1) to give the title compound (1.27 g, 90% purity from ¹H NMR, 88% yield) as white solids. LC-MS (ESI): mass calcd. for C₂₄H₃₉F₂N₃O₆ 503.3, m/z found 504.3[M+H]⁺. ¹H NMR (400 MHz, CDCl₃) δ 5.95-5.85 (m, 1H), 5.32 (d, J=17.2 Hz, 1H), 5.24 (d, J=10.4 Hz, 1H), 4.59-4.53 (m, 2.6H), 4.49-4.44 (m, 0.4H), 4.42-4.35 (m, 0.6H), 4.33-4.21 (m, 0.4H), 3.87-3.80 (m, 0.5H), 3.75-3.67 (m, 0.5H), 3.51-3.40 (m, 2H), 3.21-3.15 (m, 1H), 2.85-2.67 (m, 2H), 1.92-1.76 (m, 2H), 1.49-1.44 (m, 18H), 1.22 (s, 3H), 1.20 (s, 3H).

Racemic S28 (1.49 g, 90% purity, 2.66 mmol) was separated by chiral Prep. HPLC (Column: Chiralpak IG 5 μm 30*250 nm; Mobile Phase: Hex:EtOH=95:05 at 25 mL/min; Temp: 30° C.; Wavelength: 214 nm) to afford the title compounds S28A (603 mg, 40% yield, 90% purity from ¹H NMR, 100% stereopure) and S28B (576 mg, 39% yield, 90% purity from ¹H NMR, 100% stereopure) as white solids.

S28A: LC-MS (ESI): mass calcd. for C₂₄H₃₉F₂N₃O₆ 503.3, m/z found 504.3[M+H]⁺. Chiral analysis (Column: Chiralpak IG 5 μm 4.6*250 mm; Mobile Phase: Hex:EtOH=95:5 at 1.0 mL/min; Temp: 30° C.; Wavelength: 214 nm, R_{T}=7.565 min). ¹H NMR (400 MHz, CDCl₃) δ 5.95-5.86 (m, 1H), 5.31 (d, J=18.4 Hz, 1H), 5.23 (d, J=9.2 Hz, 1H), 4.56 (dt, J=6.0, 1.2 Hz, 2H), 4.55-4.52 (m, 0.5H), 4.49-4.44 (m, 0.5H), 4.43-4.33 (m, 0.6H), 4.32-4.23 (m, 0.4H), 3.87-3.79 (m, 0.5H), 3.75-3.68 (m, 0.5H), 3.54-3.40 (m, 2H), 3.21-3.13 (m, 1H), 2.84-2.69 (m, 2H), 191-1.77 (m, 2H), 1.49-1.44 (m, 18H), 1.22 (s, 3H), 1.20 (s, 3H).

S28B: LC-MS (ESI): mass calcd. for C₂₄H₃₉F₂N₃O₆ 503.3, m/z found 504.3[M+H]⁺. Chiral analysis (Column: Chiralpak IG 5 μm 4.6*250 mm; Mobile Phase: Hex:EtOH=95:5 at 1.0 mL/min; Temp: 30° C.; Wavelength: 214 nm, R_{T}=10.174 min). ¹H NMR (400 MHz, CDCl₃) δ 5.95-5.86 (m, 1H), 5.32 (d, J=17.2 Hz, 1H), 5.21 (d, J=10.0 Hz, 1H), 4.57-4.56 (m, 2H), 4.54-4.51 (m, 0.5H), 4.47-4.44 (m, 0.5H), 4.41-4.35 (m, 0.6H), 4.31-4.21 (m, 0.4H), 3.88-3.80 (m, 0.5H), 3.75-3.68 (m, 0.5H), 3.51-3.40 (m, 2H), 3.21-3.15 (m, 1H), 2.83-2.70 (m, 2H), 1.91-1.77 (m, 2H), 1.49-1.45 (m, 18H), 1.22 (s, 3H), 1.20 (s, 3H).

### S29B: tert-butyl (cis)-1-(4-(allyloxy)-3,3-dimethyl-4-oxobutyl)-2-ethyl-6,6-difluorohexahydropyrrolo[3,2-c]pyrazole-4(1H)-carboxylate

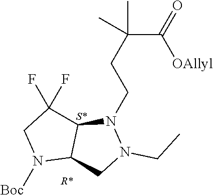

To a solution of S28B (100 mg, 90% purity, 0.179 mmol) in dichloromethane (2 mL) was added a solution of trifluoroacetic acid (0.5 mL) and dichloromethane (8 mL) at 0° C. under nitrogen atmosphere. After stirred at 0° C. for 3 hours, the mixture was poured into saturated sodium bicarbonate solution (10 mL) and extracted with dichloromethane (10 mL) for three times. The combined organic layers were washed with brine (20 mL), dried over Na₂SO₄₍ₛ₎ and concentrated to give a residue, which was dissolved in methanol (10 mL). To this solution, a mixture solution of acetaldehyde (66 mg, 1.50 mmol) in water (1.5 mL) and acetic acid glacial (0.6 mL) was added at room temperature under nitrogen atmosphere. After stirred at room temperature for 30 minutes, sodium cyanoborohydride (100 mg, 1.59 mmol) was slowly added into the mixture and stirring continued for 30 minutes. The mixture was quenched with ice water (10 mL), basified with saturated sodium bicarbonate aqueous solution to pH 8˜9 and removed methanol under reduced pressure to give a residue, which was extracted with ethyl acetate (10 mL) for three times. The combined organic phases were dried over Na₂SO₄₍ₛ₎ and filtered. The filtrate was concentrated and purified by silica gel column chromatography (petroleum ether:ethyl acetate=10:1) to give title compound (56 mg, 90% purity from ¹H NMR, 65% yield) as colorless oil. LC-MS (ESI): mass calcd. for C₂₁H₃₅F₂N₃O₄ 431.3, m/z found 432.3 [M+H]⁺. ¹H NMR (400 MHz, CDCl₃) δ 5.96-5.86 (m, 1H), 5.34-5.30 (m, 1H), 5.22 (d, J=10.4 Hz, 1H), 4.60-4.54 (m, 2.5H), 4.53-4.48 (m, 0.5H), 3.89-3.70 (m, 2H), 3.43-3.36 (m, 1H), 3.29-3.26 (m, 0.5H), 3.21-3.17 (m, 1H), 3.10-3.07 (m, 0.5H), 2.88-2.58 (m, 4H), 1.85-1.75 (m, 2H), 1.48 (s, 4H), 1.46 (s, 5H), 1.21 (s, 6H), 1.05 (t, J=7.2 Hz, 3H).

Analogously, S29A was made, LC-MS (ESI): mass calcd. for C₂₁H₃₅F₂N₃O₄ 431.3, m/z found 432.3 [M+H]⁺. ¹H NMR (400 MHz, CDCl₃) δ 5.96-5.87 (m, 1H), 5.31 (d, J=16.4 Hz, 1H), 5.21 (d, J=12.4 Hz, 1H), 4.60-4.56 (m, 2.5H), 4.54-4.47 (m, 0.5H), 3.89-3.69 (m, 2H), 3.43-3.35 (m, 1H), 3.29-3.26 (m, 0.5H), 3.22-3.18 (m, 1H), 3.12-3.06 (m, 0.5H), 2.87-2.59 (m, 4H), 1.85-1.76 (m, 2H), 1.48 (s, 4H), 1.46 (s, 5H), 1.21 (s, 6H), 1.05 (t, J=7.2 Hz, 3H).

### Compound 90: 4-((cis)-4-(((S)-5-(ethoxycarbonyl)-6-(3-fluoro-2-methylphenyl)-2-(thiazol-2-yl)-3,6-dihydropyrimidin-4-yl)methyl)-2-ethyl-6,6-difluorohexahydropyrrolo[3,2-c]pyrazol-1(2H)-yl)-2,2-dimethylbutanoic acid

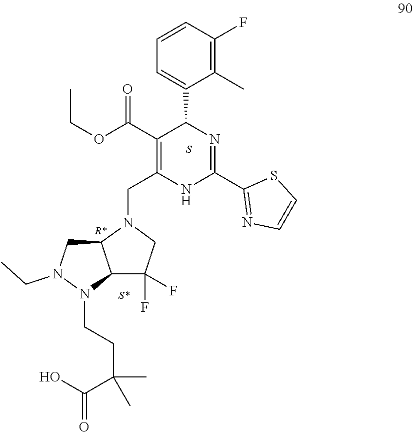

This compound was made from H12-1A and S29B according to typical coupling method 1 and typical method 2. Purified by C18 column (acetonitrile:water (+0.1% ammonium bicarbonateto)=5% to 100%) to give the title compound (47 mg, 99.5% purity, 66% yield) as yellow solids. LC-MS (ESI): mass calcd. for C₃₁H₃₉F₃N₆O₄S 648.3, m/z found 648.4 [M+H]⁺. ¹H NMR (400 MHz, CDCl₃) δ 9.06 (s, 1H), 7.76 (d, J=3.2 Hz, 1H), 7.40 (d, J=2.8 Hz, 1H), 7.11-7.05 (m, 1H), 6.98 (d, J=7.6 Hz, 1H), 6.90 (t, J=8.8 Hz, 1H), 6.01 (s, 1H), 4.32 (d, J=16.8 Hz, 1H), 4.10-3.96 (m, 4H), 3.74-3.65 (m, 1H), 3.47-3.33 (m, 3H), 2.97-2.90 (m, 2H), 2.87-2.81 (m, 2H), 2.79-2.69 (m, 1H), 2.54 (s, 1.5H), 2.53 (s, 1.5H), 1.91-1.83 (m, 1H), 1.75-1.68 (m, 1H), 1.25 (s, 3H), 1.22 (s, 3H), 1.17 (t, J=7.2 Hz, 3H), 1.10 (t, J=7.2 Hz, 3H).

### Preparation of Intermediate S57

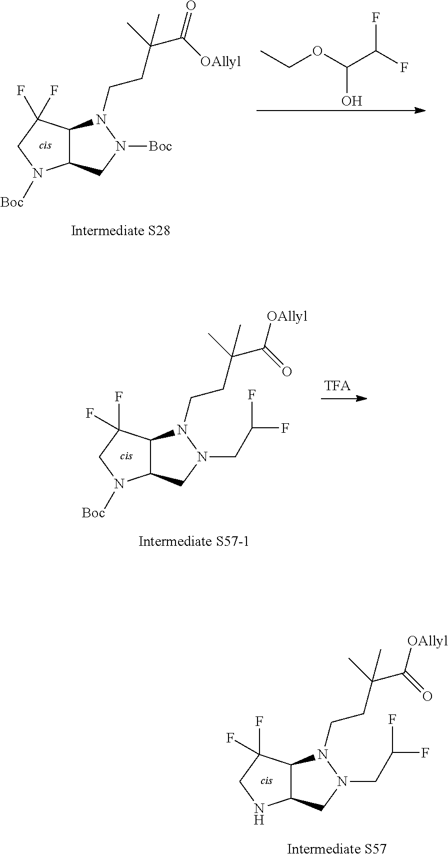

### S57-1: (cis)-tert-Butyl 1-(4-(allyloxy)-3,3-dimethyl-4-oxobutyl)-2-(2,2-difluoroethyl)-6,6-difluorohexahydropyrrolo[3,2-c]pyrazole-4(2H)-carboxylate

To a solution of S28 (400 mg, 95% purity, 0.755 mmol) in dichloromethane (40 mL) was dropwise added trifluoroacetic acid (2 mL) at 0° C. After stirred at 0° C. for 2.5 hours, the reaction mixture was poured into saturated sodium bicarbonate solution (50 mL). The organic layer was separated and the aqueous layer was extracted with dichloromethane (20 mL). The combined organic layers were washed with water (30 mL) twice, brine (30 mL) twice, dried over Na₂SO₄₍ₛ₎, filtered and concentrated to give a residue (280 mg) as colorless oil, which was diluted with methanol (5 mL) and to the mixture was added water (0.5 mL), acetic acid (0.2 mL) and 1-ethoxy-2,2-difluoroethanol (475 mg, 3.77 mmol). After stirred at room temperature for 0.5 hour, sodium cyanoborohydride (250 mg, 3.98 mmol) was added by point wise. The reaction was stirred at room temperature for 12 hours. Water (10 mL) was added to the reaction mixture, the mixture was extracted with ethyl acetate (20 mL) twice. The combined organic phases were washed by saturated sodium bicarbonate aqueous solution (50 mL) and brine (30 mL). The organic layer was dried over Na₂SO₄₍ₛ₎, filtered and concentrated to give the crude product, which was purified by C18 column (acetonitrile:water (0.5% ammonium bicarbonate=5% to 80%) to give the title compound (94 mg, 90% purity from ¹H NMR, 25% yield) as yellow oil. ¹H NMR (400 MHz, CDCl₃) δ 5.96-5.86 (m, 1.2H), 5.80-5.64 (m, 0.5H), 5.66-5.64 (m, 0.3H), 5.35-5.22 (m, 2H), 4.61-4.52 (m, 3H), 3.95-3.64 (m, 2H), 3.43-3.37 (m, 1.5H), 3.27-3.21 (m, 3.5H), 2.79-2.64 (m, 2H), 1.83-1.74 (m, 2H), 1.47 (s, 9H), 1.21 (s, 6H).

### S57: (cis)-Allyl 4-(2-(2,2-difluoroethyl)-6,6-difluorohexahydropyrrolo[3,2-c]pyrazol-1(2H)-yl)-2,2-dimethylbutanoate

To a solution of S57-1 (94 mg, 90% purity, 0.181 mmol) in dichloromethane (2 mL) was added trifluoroacetic acid (2 mL) at 0° C. After stirred at room temperature for 2 hours, the reaction mixture was concentrated under reduced pressure to give the crude product, which was purified by C18 column (acetonitrile:water (+0.5% ammonium bicarbonate)=20% to 70%) to give the title compound (73 mg, 90% purity from ¹H NMR, 99% yield) as brown oil. ¹H NMR (400 MHz, CDCl₃) δ 6.00-5.83 (m, 1.7H), 5.71-5.69 (m, 0.3H), 5.35-5.21 (m, 2H), 4.57-4.56 (m, 2H), 4.27-4.24 (m, 1H), 3.46-3.11 (m, 6H), 2.84-2.69 (m, 2H), 2.63-2.56 (m, 1H), 1.86-1.72 (m, 2H), 1.21 (s, 6H).

### Compound 91: 4-((cis)-2-(2,2-Difluoroethyl)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-c]pyrazol-1(2H)-yl)-2,2-dimethylbutanoic acid

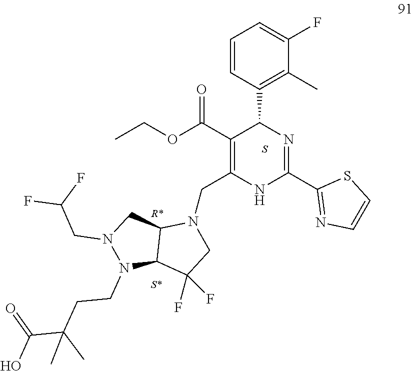

This compound was made from S57 and H2-1A according to typical method 1 and 2 successively. Chiral separation of allyl ester intermediate: chiral Prep. HPLC (Column: Column Chiralpak IG 5 μm 20*250 mm; Mobile Phase: Hex:EtOH:DEA=80:20:0.2 at 10 mL/min; Temp 30° C.; Wavelength 254 nm).

Compound 91: LC-MS (ESI): mass calcd. for C₃₁H₃₇F₅N₆O₄S 684.3, m/z found 684.8 [M+H]⁺. ¹H NMR (400 MHz, CDCl₃+one drop of D₂O) δ 7.77 (s, 1H), 7.39 (s, 1H), 7.10-7.05 (m, 1H), 7.00-6.98 (m, 1H), 6.93-6.88 (m, 1H), 6.14-6.12 (m, 0.2H), 6.02-5.96 (m, 1.6H), 5.86-5.84 (m, 0.2H), 4.42 (d, J=16.4 Hz, 1H), 4.06-3.98 (m, 2H), 3.92-3.88 (m, 3H), 3.74-3.68 (m, 1H), 3.38-3.27 (m, 2H), 3.22-3.15 (m, 1H), 3.09-3.06 (m, 1H), 2.86-2.60 (m, 3H), 2.53 (s, 3H), 1.91-1.74 (m, 2H), 1.24 (s, 6H), 1.10 (t, J=7.2 Hz, 3H).

### Preparation of Intermediate S60

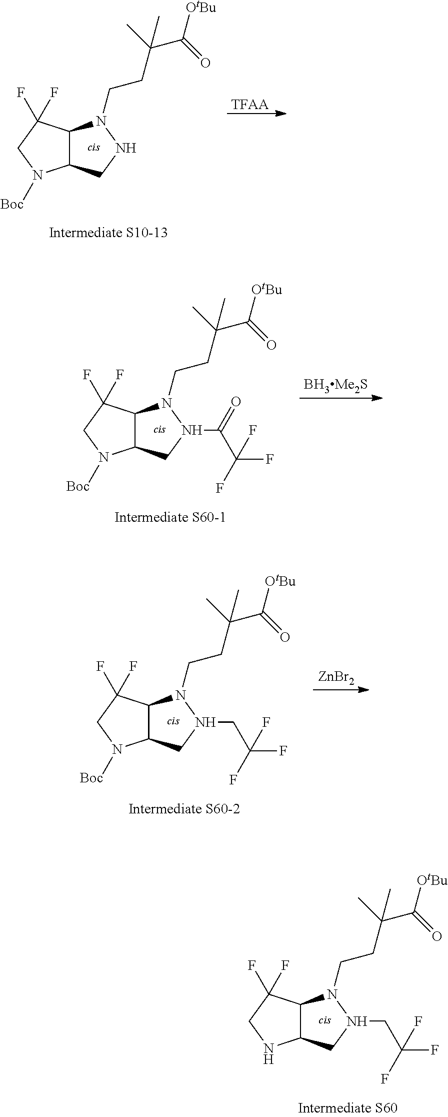

### S60-1: (cis)-tert-Butyl 1-(4-(tert-butoxy)-3,3-dimethyl-4-oxobutyl)-6,6-difluoro-2-(2,2,2-trifluoroacetyl)hexahydropyrrolo[3,2-c]pyrazole-4(2H)-carboxylate

To a solution of S10-13 (500 mg, 70% purity, 0.834 mmol) in dichloromethane (10 mL) was added triethylamine (1.3 g, 12.8 mmol) at 0° C., then trifluoroacetic anhydride (1.2 g, 5.713 mmol) was added slowly. The mixture was warmed to room temperature and stirred for 1 hour. The mixture was concentrated and the residue was purified by silica gel column chromatography (petroleum ether:ethyl acetate=30:1) to afford the desired product (320 mg, 90% purity from ¹H NMR, 67% yield) as colorless oil. ¹H NMR (400 MHz, CDCl₃) δ 4.84-4.64 (m, 1H), 4.59-4.39 (m, 1H), 3.99-3.71 (m, 2H), 3.63-3.35 (m, 2H), 2.99-2.77 (m, 2H), 1.88-1.66 (m, 2H), 1.49-1.44 (m, 18H), 1.18-1.13 (m, 6H).

### S60-2: (cis)-tert-Butyl 1-(4-(tert-butoxy)-3,3-dimethyl-4-oxobutyl)-6,6-difluoro-2-(2,2,2-trifluoroethyl)hexahydropyrrolo[3,2-c]pyrazole-4(2H)-carboxylate

To a solution of S60-1 (320 mg, 90% purity, 0.559 mmol) in tetrahydrofuran (10 mL) was added borane-methyl sulfide complex (2 mL) and boron trifluoride diethyl etherate (1 mL) at 0° C., then the reaction was heated at 30° C. overnight. The mixture was cooled to 0° C., quenched with methanol (6 mL). The mixture was stirred at 0° C. for 30 minutes, then poured to saturated sodium bicarbonate solution (50 mL), extracted with ethyl acetate (60 mL) twice. The combined organic layers were washed with brine (60 mL), dried over Na₂SO₄₍ₛ₎, filtered and concentrated. The residue was purified by silica gel column chromatography (petroleum ether:ethyl acetate=20:1) to afford the desired product (180 mg, 90% purity from ¹H NMR, 58% yield) as colorless oil. ¹H NMR (400 MHz, CDCl₃) δ 4.63-4.50 (m, 1H), 3.97-3.61 (m, 2H), 3.51-3.19 (m, 5H), 2.83-2.66 (m, 2H), 1.86-1.64 (m, 2H), 1.48 (s, 9H), 1.43 (s, 9H), 1.14 (s, 3H), 1.13 (s, 3H).

### S60: tert-Butyl 4-((cis)-6,6-difluoro-2-(2,2,2-trifluoroethyl)hexahydropyrrolo[3,2-c]pyrazol-1(2H)-yl)-2,2-dimethylbutanoate

To a solution of S60-2 (180 mg, 90% purity, 0.323 mmol) in dichloromethane (10 mL) was added zinc(II) bromide (700 mg, 3.11 mmol) at room temperature. After stirred at room temperature overnight, the mixture was poured to saturated sodium carbonate solution (20 mL), extracted with dichloromethane (50 mL) twice. The combined organic layers were washed with brine (50 mL), dried over Na₂SO₄₍ₛ₎, filtered and concentrated to afford the desired product (150 mg, 55% purity, 64% yield) as colorless oil. LC-MS (ESI): mass calcd. for C₁₇H₂₈F₅N₃O₂ 401.4, m/z found 402.3 [M+H]⁺.

### Compound 92: 4-((cis)-4-(((S)-5-(Ethoxycarbonyl)-6-(3-fluoro-2-methylphenyl)-2-(thiazol-2-yl)-3,6-dihydropyrimidin-4-yl)methyl)-6,6-difluoro-2-(2,2,2-trifluoroethyl)hexahydropyrrolo[3,2-c]pyrazol-1(2H)-yl)-2,2-dimethylbutanoic acid

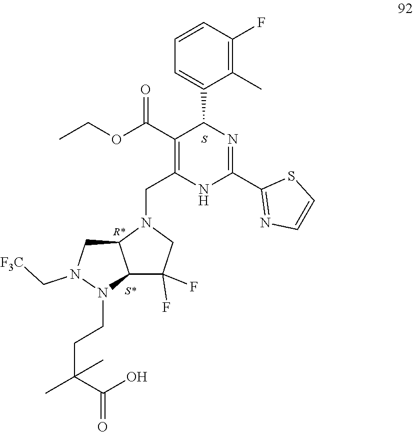

This compound was made from S60 and H2-1A according to typical method 1 and 3 successively. Chiral separation of tert-Butyl ester compounds: Chiral Prep-HPLC (Column: Chiralpak IG 5 μm 25 mm*250 mm; Mobile Phase: Hex:EtOH=90:10 at 25 mL/min; Temp: 30° C.; Wavelength: 254 nm)

Compound 92, LC-MS (ESI): mass calcd. for C₃₁H₃₆F₆N₆O₄S 702.2, m/z found 703.3 [M+H]⁺. ¹H NMR (400 MHz, CD₃OD) δ 7.77 (br s, 1H), 7.66 (br s, 1H), 7.15-7.06 (m, 2H), 6.95-6.83 (m, 1H), 5.95 (s, 1H), 4.27 (d, J=16.0 Hz, 1H), 4.03-3.98 (m, 3H), 3.95-3.83 (m, 2H), 3.78-3.64 (m, 2H), 3.24-3.15 (m, 3H), 2.90-2.72 (m, 2H), 2.66-2.57 (m, 1H), 2.47 (s, 3H), 1.89-1.64 (m, 2H), 1.16 (s, 3H), 1.15 (s, 3H), 1.07 (t, J=7.2 Hz, 3H).

### Preparation of Intermediate S36

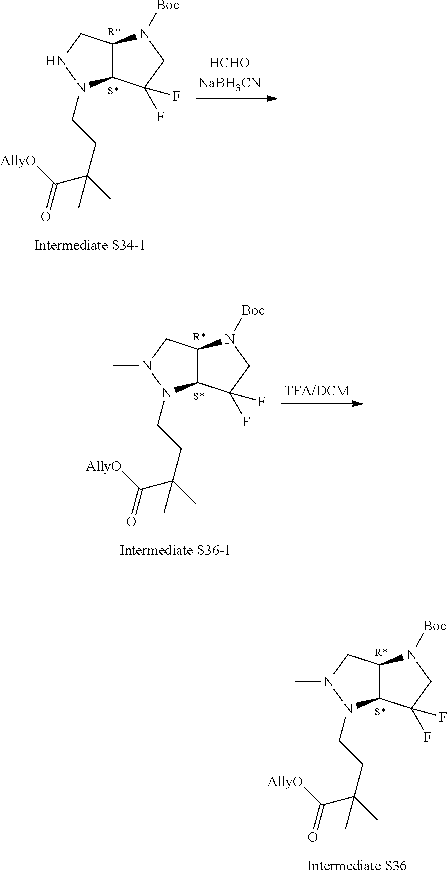

### S36-1: (cis)-tert-Butyl 1-(4-(allyloxy)-3,3-dimethyl-4-oxobutyl)-6,6-difluoro-2-methylhexahydropyrrolo[3,2-c]pyrazole-4(2H)-carboxylate

To a solution of S34-1 (100 mg, crude) in MeOH (10 mL) was added acetic acid glacial (1 mL) and 37% formaldehyde aqueous solution (0.2 mL, 1.48 mmol) under nitrogen atmosphere. After stirred at 25° C. for 2 hours, sodium cyanoborohydride (45 mg, 0.716 mmol) was added. After stirred at 25° C. for another 1 hour, the reaction mixture was quenched with saturated sodium bicarbonate aqueous solution (10 mL), extracted with dichloromethane (10 mL) for three times. The combined organic phases were washed with brine (10 mL), dried over Na₂SO₄₍ₛ₎ and filtered. The filtrate was concentrated to give a residue, which was purified by silica gel column chromatography (petroleum ether:ethyl acetate=5:1) to give the title compound (57 mg, 90% purity, 65% yield) as light yellow oil. ¹H NMR (400 MHz, CDCl₃) δ 5.96-5.86 (m, 1H), 5.32 (dd, J=17.2, 1.6 Hz, 1H), 5.23 (d, J=10.4 Hz, 1H), 4.58-4.49 (m, 3H), 4.38 (s, 1H), 3.89-3.77 (m, 2H), 3.40-3.29 (m, 2H), 2.78-2.66 (m, 2H), 2.54 (s, 3H), 1.86-1.80 (m, 2H), 1.47-1.46 (m, 9H), 1.22 (s, 6H).

### S36: allyl 4-((cis)-6,6-difluoro-2-methylhexahydropyrrolo[3,2-c]pyrazol-1(2H)-yl)-2,2-dimethylbutanoate

To a solution of S36-1 (57 mg, 90% purity, 0.123 mmol) in dichloromethane (10 mL) was added trifluoroacetic acid (2 mL) at room temperature. After stirred at room temperature for 1.5 hours, the mixture was quenched with saturated sodium bicarbonate aqueous solution (10 mL). It was extracted with dichloromethane (10 mL) twice. The combined extracts were concentrated to give a residue, which was used in next step directly (not stable on storage).

### Compound 93: 4-(4-((6-(3,4-difluoro-2-methylphenyl)-5-(ethoxycarbonyl)-2-(thiazol-2-yl)-3,6-dihydropyrimidin-4-yl)methyl)-6,6-difluoro-2-methylhexahydropyrrolo[3,2-c]pyrazol-1(2H)-yl)-2, ethylbutanoic acid (Single Diastereomer)

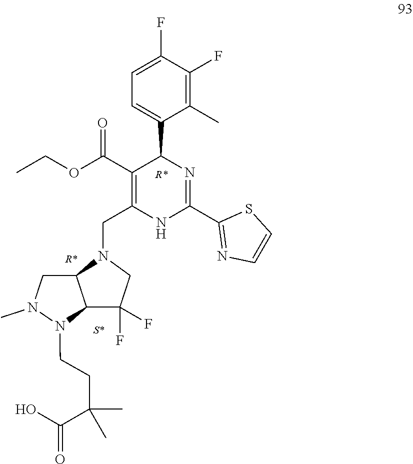

This compound was made from H9-1A and S36 according to typical coupling method 1 and typical method 2 successively. Purified with C18 column (acetonitrile:water (5% ammonium bicarbonate)=5% to 100%) to afford the desired product (20 mg, 97.2% purity, 28% yield) as yellow solids. LC-MS (ESI): mass calcd. for C₃₀H₃₆F₄N₆O₄S 652.2, m/z found 653.4 [M+H]⁺. ¹H NMR (400 MHz, CDCl₃) δ 7.89 (d, J=2.8 Hz, 1H), 7.72 (d, J=2.8 Hz, 1H), 7.07-7.02 (m, 2H), 5.94 (s, 1H), 4.32-4.14 (m, 2H), 4.10-4.05 (m, 3H), 3.74-3.66 (m, 1H), 3.50-3.40 (m, 1H), 3.27-3.22 (m, 1H), 3.01-2.86 (m, 3H), 2.75 (s, 3H), 2.72-2.64 (m, 1H), 2.56 (s, 3H), 1.86-1.78 (m, 2H), 1.22 (s, 6H), 1.14 (t, J=7.2 Hz, 3H).

### Compound 94: 4-((cis)-4-(((R)-6-(2-chloro-4-fluorophenyl)-5-(methoxycarbonyl)-2-(thiazol-2-yl)-3,6-dihydropyrimidin-4-yl)methyl)-6,6-difluoro-2-methylhexahydropyrrolo[3,2-c]pyrazol-1(2H)-yl)-2,2-dimethylbutanoic acid

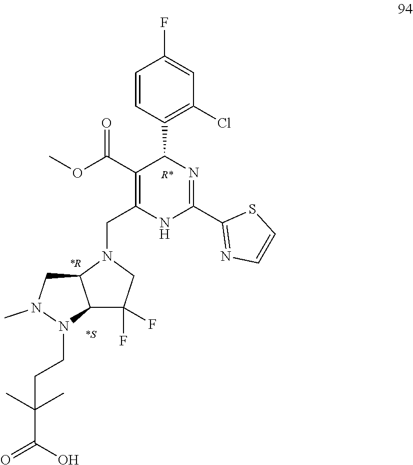

This compound was made from H3-1A and S10 analogous to compound 68A. LC-MS (ESI): mass calcd. for C₂₈H₃₂ClF₃N₆O₄S 640.18, m/z found 641.3 [M+H]⁺. ¹H NMR (400 MHz, CD₃OD) δ 7.88 (m, 1H), 7.73 (m, 1H), 7.47-7.38 (m, 1H), 7.28-7.20 (m, 1H), 7.08-7.01 (m, 1H), 6.15 (s, 1H), 4.17-4.08 (m, 1H), 4.06-3.99 (m, 1H), 3.73-3.64 (m, 1H), 3.59 (s, 3H), 3.49-3.37 (m, 2H), 3.19-3.09 (m, 1H), 3.03-2.83 (m, 3H), 2.74 (s, 3H), 2.68-2.58 (m, 1H), 1.86-1.72 (m, 2H), 1.24-1.16 (m, 6H).

### Preparation of Intermediate S41

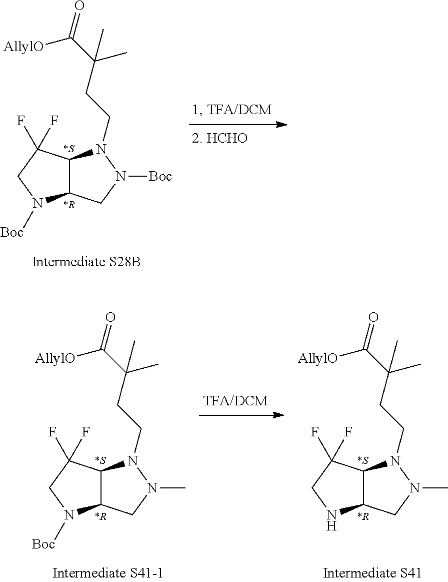

### S41-1: (cis)-tert-Butyl 1-(4-(allyloxy)-3,3-dimethyl-4-oxobutyl)-6,6-difluoro-2-methylhexahydropyrrolo[3,2-c]pyrazole-4(2H)-carboxylate

To a solution of S28B (360 mg, 90% purity, 0.64 mmol) in dichloromethane (10 mL) was added trifluoroacetic acid (0.5 mL) at 0° C. After stirred at 0° C. for 3 hours, the mixture was quenched with saturated sodium bicarbonate aqueous solution (10 mL), extracted with dichloromethane (10 mL) twice. The combined extracts were concentrated to give a residue, which was dissolved in methanol (10 mL). To this solution, acetic acid glacial (1 mL) and 37% formaldehyde aqueous solution (0.5 mL, 5.02 mmol) were added under nitrogen atmosphere. The reaction was stirred at 25° C. for 2 hours, then sodium cyanoborohydride (150 mg, 2.39 mmol) was added. After stirred at 25° C. for 1 hour, the reaction mixture was quenched with saturated sodium bicarbonate aqueous solution (10 mL), extracted with dichloromethane (10 mL) for three times. The combined organic phases were washed with brine (10 mL), dried over Na₂SO₄₍ₛ₎ and filtered. The filtrate was concentrated to give a residue, which was purified by silica gel column chromatography (petroleum ether:ethyl acetate=5:1) to give the title compound (215 mg, 90% purity from ¹H NMR, 72% yield) as light yellow oil. LC-MS (ESI): mass calcd. for C₂₀H₃₃F₂N₃O₄ 417.2, m/z found 418.3 [M+H]⁺. ¹H NMR (400 MHz, CDCl₃) δ 5.96-5.86 (m, 1H), 5.32 (dd, J=17.6, 2.0 Hz, 1H), 5.22 (dd, J=10.8, 1.2 Hz, 1H), 4.57 (dt, J=5.6, 1.6 Hz, 2.5H), 4.50-4.46 (m, 0.5H), 3.92-3.71 (m, 2H), 3.41-3.29 (m, 2H), 3.05-3.00 (m, 0.5H), 2.85-2.65 (m, 2.5H), 2.54 (s, 3H), 1.47-1.46 (m, 9H), 1.22 (s, 6H).

### S41: Allyl 4-((cis)-6,6-difluoro-2-methylhexahydropyrrolo[3,2-c]pyrazol-1(2H)-yl)-2,2-dimethylbutanoate

To a solution of S41-1 (130 mg, 90% purity, 0.280 mmol) in dichloromethane (2 mL) was added trifluoroacetic acid (1 mL) at room temperature. After stirred at room temperature under nitrogen atmosphere for 2 hours, the reaction mixture was diluted with saturated sodium bicarbonate aqueous solution (4 mL) and extracted with dichloromethane (3 mL) twice. The combined extracts were washed with brine (5 mL), dried over Na₂SO₄₍ₛ₎, filtered and concentrated to give the desired compound (90 mg, 90% purity from ¹H NMR, 91% yield) as light yellow oil. ¹H NMR (400 MHz, CDCl₃) δ 5.97-5.87 (m, 1H), 5.32 (dd, J=17.6, 1.6 Hz, 1H), 5.22 (dd, J=10.4, 1.2 Hz, 1H), 4.57 (d, J=6.0 Hz, 2H), 4.20-4.13 (m, 1H), 3.32-3.05 (m, 3.5H), 2.86-2.70 (m, 2H), 2.43-2.36 (m, 3.5H), 2.17 (s, 1H), 1.87-1.75 (m, 2H), 1.22 (s, 6H).

### Compound 95: 4-((cis)-4-((6-(2-Chloro-3,4-difluorophenyl)-5-(methoxycarbonyl)-2-(thiazol-2-yl)-3,6-dihydropyrimidin-4-yl)methyl)-6,6-difluoro-2-methylhexahydropyrrolo[3,2-c]pyrazol-1(2H)-yl)-2,2-dimethylbutanoic acid

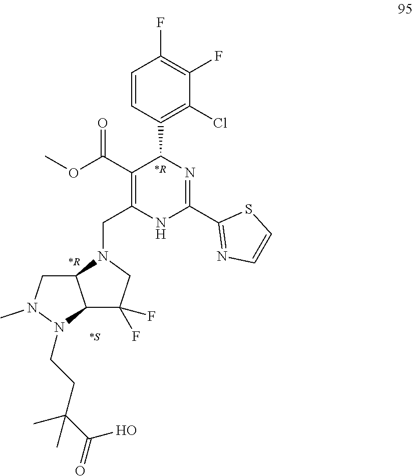 [SMILES: COC(=O)C1=C(CN2CC(F)(F)[C@@H]3[C@H]2CN(C)N3CCC(C)(C)C(=O)O)NC(c2nccs2)=N[C@H]1c1ccc(F)c(F)c1Cl]

This compound was made from H5-1A and S41 according to typical method 1 and typical method 2. Purified by C18 column (acetonitrile:water (+0.1% ammonium bicarbonateto)=5% to 100%) to to give the title compound (26.1 mg, 96.9% purity, 61% yield) as yellow solids. LC-MS (ESI): mass calcd. for C₂₈H₃₁ClF₄N₆O₄S 658.2, m/z found 659.2 [M+H]⁺. ¹H NMR (400 MHz, CD₃OD) δ 9.25 (s, 1H), 7.81 (d, J=3.2 Hz, 1H), 7.44 (d, J=2.8 Hz, 1H), 7.09-7.00 (m, 2H), 6.18 (s, 0.9H), 6.07 (s, 0.1H), 4.26-4.08 (m, 3H), 3.69-3.61 (m, 1H), 3.59 (s, 3H), 3.50-3.41 (m, 1H), 3.38-3.34 (m, 1H), 3.02-2.94 (m, 2H), 2.83-2.76 (m, 2H), 2.73 (s, 3H), 1.82-1.79 (m, 2H), 1.25 (s, 3H), 1.23 (s, 3H).

### Compound 96: 4-((cis)-4-(((R)-5-(ethoxycarbonyl)-6-(6-fluoro-2-methylpyridin-3-yl)-2-(thiazol-2-yl)-3,6-dihydropyrimidin-4-yl)methyl)-6,6-difluoro-2-methylhexahydropyrrolo[3,2-c]pyrazol-1(2H)-yl)-2,2-dimethylbutanoic acid

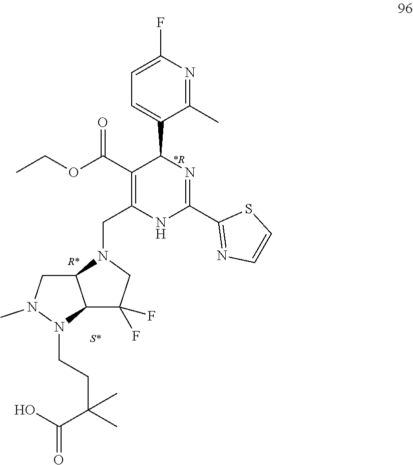 [SMILES: CCOC(=O)C1=C(CN2CC(F)(F)[C@@H]3[C@H]2CN(C)N3CCC(C)(C)C(=O)O)NC(c2nccs2)=N[C@@H]1c1ccc(F)nc1C]

This compound was made from H20-1A and S41 according to typical method 1 and 2. LC-MS (ESI): mass calcd. for C₂₉H₃₆F₃N₇O₄S 635.3, m/z found 636.3 [M+H]⁺. ¹H NMR (400 MHz, CDCl₃) δ 9.22 (s, 1H), 7.80 (d, J=3.2 Hz, 1H), 7.54 (t, J=8.0 Hz, 1H), 7.43 (d, J=3.2 Hz, 1H), 6.71-6.68 (m, 1H), 5.98 (s, 1H), 4.27 (d, J=17.2 Hz, 1H), 4.16-4.01 (m, 4H), 3.70-3.63 (m, 1H), 3.50-3.43 (m, 1H), 3.41-3.36 (m, 1H), 3.34-3.29 (m, 1H), 3.02-2.94 (m, 2H), 2.82-2.78 (m, 4H), 2.75-2.68 (m, 4H), 1.80 (t, J=8.0 Hz, 2H), 1.24 (s, 3H), 1.22 (s, 3H), 1.14 (t, J=7.2 Hz, 3H).

### Preparation of Intermediate S55

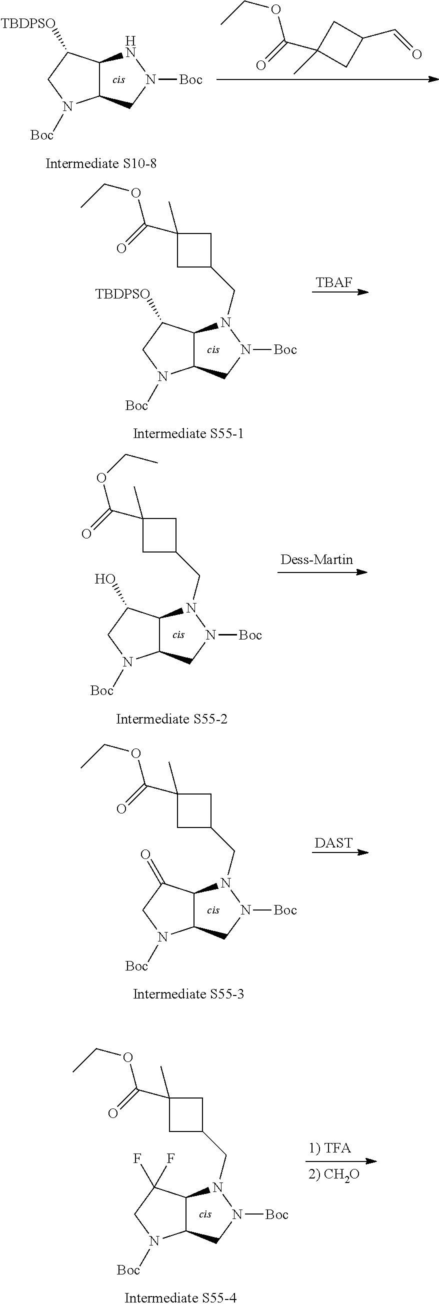

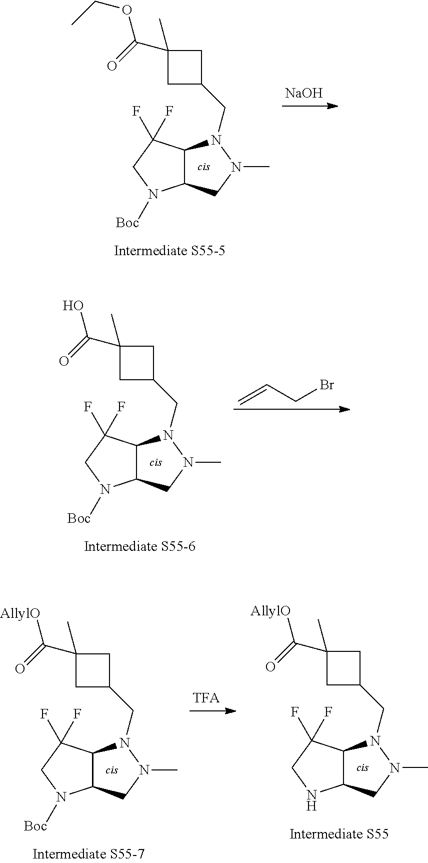

### S55-1: (cis)-di-tert-Butyl 6-((tert-butyldiphenylsilyl)oxy)-(ethoxycarbonyl)-3-methylcyclobutyl)methyl)hexahydropyrrolo[3,2-c]pyrazole-2,4-dicarboxylate

A solution of (cis)-di-tert-butyl 6-((tert-butyldiphenylsilyl)oxy)hexahydropyrrolo[3,2-c]pyrazole-2,4-dicarboxylate S10-8 (3.8 g, 90% purity, 6.02 mmol), glacial acetic acid (3.8 mL) and ethyl 3-formyl-1-methylcyclobutanecarboxylate (1.8 g, 90% purity, 9.52 mmol) in 1,2-dichloroethane (38 mL) was stirred at 30° C. for 3 hours. Then sodium triacetoxyhydroborate (3.2 g, 15.1 mmol) was added and the mixture was stirred at 25° C. for 3 hours. The mixture was poured into water (50 mL) and extracted with dichloromethane (30 mL) for three times. The combined organic layers were washed with brine (30 mL) and concentrated to get the crude, which was purified by silica gel column chromatography (petroleum ether:ethyl acetate=10:1) to get the desired compound (3.6 g, 83% purity, 69% yield) as white solids. LC-MS (ESI): mass calcd. For C₄₀H₅₉N₃O₇Si 721.4, m/z found 722.6 [M+H]⁺.

### S55-2: (cis)-di-tert-Butyl 1-((3-(ethoxycarbonyl)-3-methylcyclobutyl)methyl)-6-hydroxyhexahydropyrrolo[3,2-c]pyrazole-2,4-dicarboxylate

To a solution of S55-1 (4.2 g, 83% purity, 4.83 mmol) in tetrahydrofuran (5 mL) was added 1.0 M tetrabutylammonium fluoride in tetrahydrofuran (15 mL, 15 mmol). After stirred at room temperature for 4 hours, the mixture was poured into water (20 mL) and extracted with ethyl acetate (30 mL) for three times. The combined organic layers were washed with brine (30 mL) and concentrated to get the crude, which was purified by silica gel column chromatography (petroleum ether:ethyl acetate=2:1) to get the desired compound (3.0 g, 75% purity, 96% yield) as colorless oil. ¹H NMR (300 MHz, CDCl₃) δ 4.62-4.46 (m, 1H), 4.25-4.12 (m, 4H), 3.50-3.24 (m, 4H), 2.90-2.57 (m, 4H), 2.32-1.92 (m, 4H), 1.52-1.38 (m, 19.5H), 1.38 (s, 1.5H), 1.30-1.27 (m, 3H).

### S55-3: (cis)-di-tert-Butyl 1-((3-(ethoxycarbonyl)-3-methylcyclobutyl)methyl)-6-oxohexahydropyrrolo[3,2-c]pyrazole-2,4-dicarboxylate

To a solution of S55-2 (3.0 g, 75% purity, 4.65 mmol) in dichloromethane (40 mL) was added Dess-Martin periodinane (3.4 g, 8.02 mmol). After stirred at 25° C. for 4 hours, the reaction mixture was quenched by 2 M sodium bicarbonate aqueous solution (30 mL) and 2 M sodium thiosulfate aqueous solution (30 mL). The mixture was extracted with dichloromethane (30 mL) for three times. The combined organic layers were washed with brine (30 mL) and concentrated to get the crude, which was purified by silica gel column chromatography (petroleum ether:ethyl acetate=4:1) to get the desired compound (2.5 g, 85% purity from ¹H NMR, 95% yield) as yellow oil. ¹H NMR (400 MHz, CDCl₃) δ 4.79-4.70 (m, 1H), 4.49-4.34 (m, 1H), 4.20-4.09 (m, 2H), 3.83-3.66 (m, 2H), 3.43-3.26 (m, 2H), 2.86-2.74 (m, 2H), 2.68-2.54 (m, 2H), 2.22-2.13 (m, 1H), 2.02-1.90 (m, 1H), 1.74-1.63 (m, 1H), 1.51-1.41 (m, 19.5H), 1.33 (s, 1.5H), 1.27-1.25 (m, 3H).

### S55-4: (cis)-di-tert-Butyl 1-((3-(ethoxycarbonyl)-3-methylcyclobutyl)methyl)-6,6-difluorohexahydropyrrolo[3,2-c]pyrazole-2,4-dicarboxylate

To a solution of S55-3 (2.0 g, 85% purity, 3.53 mmol) in dichloromethane (30 mL) was added diethylaminosulfurtrifluoride (3.2 g, 19.9 mmol) under −78° C. After stirred at room temperature for 4 hours, the mixture was poured into 2 M sodium bicarbonate aqueous solution (100 mL) and extracted with dichloromethane (40 mL) for three times. The combined organic layers were washed with brine (40 mL) and concentrated to get the crude, which was purified by silica gel column chromatography (petroleum ether:ethyl acetate=10:1) to get the desired compound (1.7 g, 88% purity, 84% yield) as yellow oil. LC-MS (ESI): mass calcd. for C₂₄H₃₉F₂N₃O₆ 503.28, m/z found 504.4 [M+H]⁺.

### S55-5: (cis)-tert-Butyl 1-((3-(ethoxycarbonyl)-3-methylcyclobutyl)methyl)-6,6-difluoro-2-methylhexahydropyrrolo[3,2-c]pyrazole-4(2H)-carboxylate

To a solution of S55-4 (900 mg, 88% purity, 1.57 mmol) in dichloromethane (40 mL) was added trifluoroacetic acid (2 mL) under ice bath. After stirred at 0° C. for 3 hours, the mixture was basified with 2 M sodium bicarbonate aqueous solution to pH=9 and extracted with dichloromethane (30 mL) for three times. The combined organic layers were washed with brine (30 mL) and concentrated to get the crude, which was dissolved in methanol (10 mL). Then glacial acetic acid (0.7 mL), 37% of formaldehyde aqueous solution (1.2 mL, 15.8 mmol) were added. The mixture was stirred at 25° C. for 0.5 hours. Then sodium cyanoborohydride (490 mg, 7.80 mmol) was added and the mixture and stirred at 25° C. for 3 hours. The mixture was poured into water (20 mL) and extracted with dichloromethane (20 mL) for three times. The combined organic layers were washed with brine (20 mL) and concentrated to get the crude, which was purified by silica gel column chromatography (petroleum ether:ethyl acetate=5:1) to get the desired compound (650 mg, 88% purity, 87% yield) as yellow oil. LC-MS (ESI): mass calcd. for C₂₀H₃₃F₂N₃O₄ 417.2, m/z found 418.5 [M+H]⁺.

### S55-6: (cis)-3-((4-(tert-Butoxycarbonyl)-6,6-difluoro-2-methylhexahydropyrrolo[3,2-c]pyrazol-1(2H)-yl)methyl)-1-methylcyclobutanecarboxylic acid

To a solution of S55-5 (650 mg, 88% purity, 1.37 mmol) in ethanol (5 mL) and water (2 mL) was added sodium hydroxide (170 mg, 4.25 mmol). The mixture was stirred at 35° C. for 12 hours. Then it was acidified with 1 M hydrochloride aqueous solution to to pH=3 and extracted with ethyl acetate (30 mL) for three times. The combined organic layers were washed with brine (20 mL) and concentrated to get the desired compound (500 mg, 86% purity, 81% yield) as yellow oil. LC-MS (ESI): mass calcd. for C₁₈H₂₉F₂N₃O₄, 389.2, m/z found 390.6 [M+H]⁺.

### S55-7: (cis)-tert-Butyl 1-((3-((allyloxy)carbonyl)-3-methylcyclobutyl)methyl)-6,6-difluoro-2-methylhexahydropyrrolo[3,2-c]pyrazole-4(2H)-carboxylate

To a mixture of S55-6 (500 mg, 86% purity, 1.10 mmol) and potassium carbonate (460 mg, 1.16 mmol) in N,N-dimethylformide (8 mL) was added 3-bromoprop-1-ene (270 mg, 2.23 mmol). The mixture was stirred at room temperature for 12 hours. The mixture was poured into water (30 mL) and extracted with dichloromethane (10 mL) for three times. The combined organic layers were washed with brine (10 mL) and concentrated to get the crude, which was purified by silica gel column chromatography (petroleum ether:ethyl acetate=5:1) to get the desired compound (400 mg, 92% purity, 77% yield) as colorless oil. LC-MS (ESI): mass calcd. for C₂₁H₃₃F₂N₃O₄, 429.2, m/z found 430.3 [M+H]⁺.

### S55: (cis)-Allyl 3-((6,6-difluoro-2-methylhexahydropyrrolo[3,2-c]pyrazol-1(2H)-yl)methyl)-1-methylcyclobutanecarboxylate

To a solution of S55-7 (400 mg, 92% purity, 0.86 mmol) in dichloromethane (4 mL) was added trifluoroacetic acid (2 mL). After stirred at room temperature for 3 hours, the mixture was basified with 2 M sodium bicarbonate aqueous solution to pH=9 and extracted with dichloromethane (10 mL) for three times. The combined organic layers were washed with brine (10 mL) and concentrated to get the desired compound (280 mg, 97% purity, 96% yield) as yellow oil. LC-MS (ESI): mass calcd. for C₁₆H₂₅F₂N₃O₂, 329.2, m/z found 330.3 [M+H]⁺.

### Compound 98: 3-(((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluoro-2-methylhexahydropyrrolo[3,2-c]pyrazol-1(2H)-yl)methyl)-1-methylcyclobutane-1-carboxylic acid

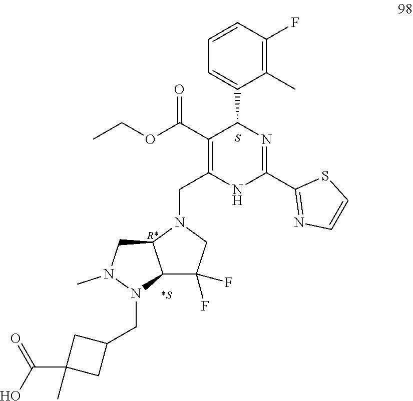

This compound was made from H2-1A and S55 according to typical method 1 and 2 successively. Chiral separation method of allyl ester compounds: chiral Prep. HPLC (Column: ChiralPak IC 5 μm 20*250 mm; Mobile Phase: Hex:IPA=90:10 at 25 mL/min; Temp: 30° C.; Wavelength: 214 nm).

Compound 98: LC-MS (ESI): mass calcd. for C₃₁H₃₇F₃N₆O₄S 646.2, m/z found 647.3 [M+H]⁺. ¹H NMR (400 MHz, CDCl₃) δ 9.14-9.13 (m, 1H), 7.78-7.76 (m, 1H), 7.39 (d, J=3.6 Hz, 1H), 7.10-7.05 (m, 1H), 6.99-6.97 (m, 1H), 6.92-6.88 (m, 1H), 6.01 (s, 1H), 4.29-4.24 (m, 1H), 4.15-3.97 (m, 4H), 3.55-3.41 (m, 2H), 3.28-3.23 (m, 1H), 3.00-2.80 (m, 3H), 2.72-2.69 (m, 3H), 2.69-2.58 (m, 1H), 2.54-2.53 (m, 3H), 2.29-2.20 (m, 2H), 2.07-2.02 (m, 2H), 1.72-1.69 (m, 1H), 1.46 (s, 1.7H), 1.38 (s, 1.3H), 1.11 (t, J=6.8 Hz, 3H).

### Preparation of Intermediate S45

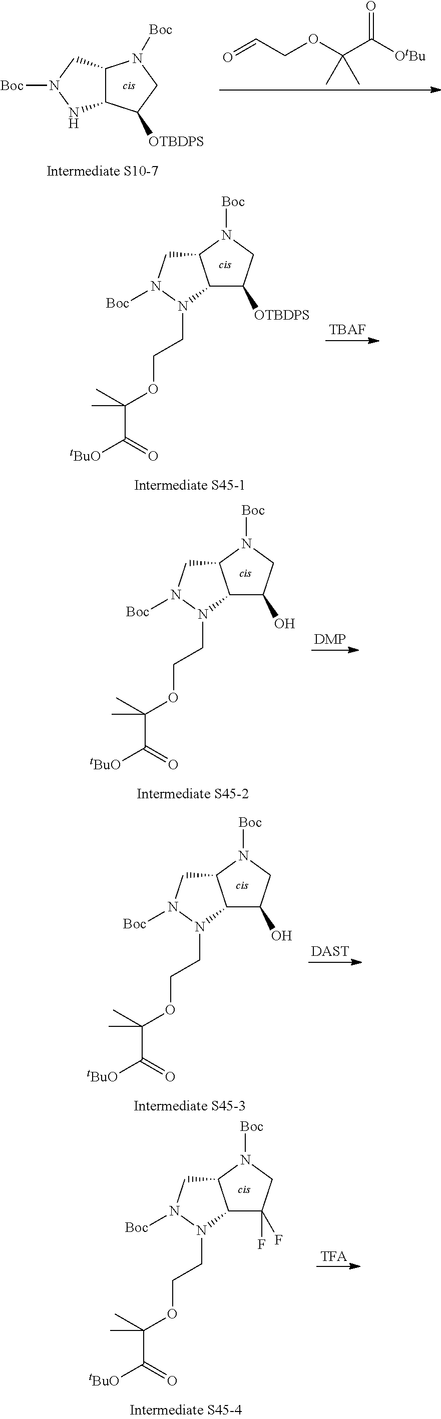

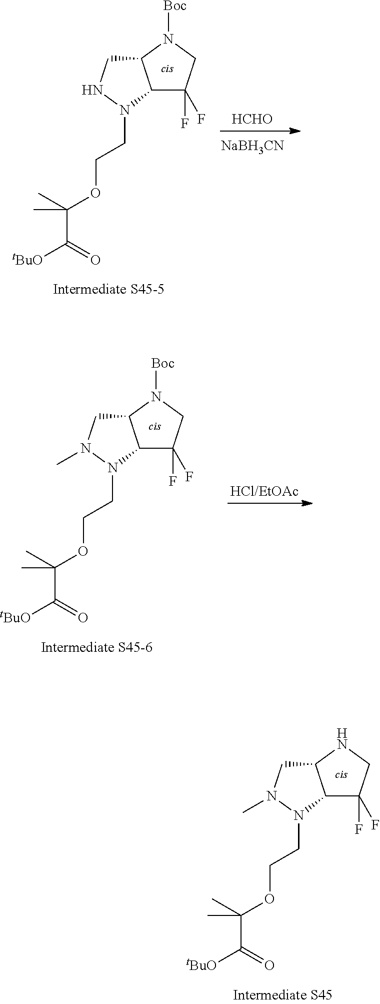

### S45-1: (cis)-Di-tert-butyl 1-(2-((1-(tert-butoxy)-2-methyl-1-oxopropan-2-yl)oxy)ethyl)-6-((tert-butyldiphenylsilyl)oxy)hexahydropyrrolo[3,2-c]pyrazole-2,4-dicarboxylate

To a solution of S10-7 (4.00 g, 90% purity, 6.34 mmol) in methanol (160 mL) were added tert-butyl 2-methyl-2-(2-oxoethoxy)propanoate (4.00 g, 80% purity, 15.8 mmol) and acetic acid (16 mL) at room temperature. The mixture was stirred at room temperature overnight, then sodium cyanotrihydroborate (1.6 g, 25.5 mmol) was added into the mixture. After stirred at room temperature for 5 hours under nitrogen atmosphere, the mixture was quenched with 1 M hydrochloride aqueous solution (30 mL) and dichloromethane (30 mL). The aqueous layer was extracted with dichloromethane (30 mL) for three times. The combined organic layers were washed with brine (30 mL), dried over Na₂SO₄₍ₛ₎ and filtered. The filtrate was concentrated under reduced pressure to give a residue, which was purified by C18 column (acetonitrile:water=5% to 95%) to give the title compound (4.4 g, 90% purity from ¹H NMR, 83% yield) as yellow oil. LC-MS (ESI): mass calcd. for C₄₁H₆₃N₃O₈Si 753.4, m/z found 754.7 [M+H]⁺. ¹H NMR (400 MHz, CDCl₃) δ 7.67-7.58 (m, 4H), 7.46-7.34 (m, 6H), 4.62-4.58 (m, 0.5H), 4.46-4.42 (m, 0.5H), 4.28-4.11 (m, 2H), 3.53-3.38 (m, 4H), 3.26-3.18 (m, 1H), 3.08-3.05 (m, 0.5H), 2.81-2.65 (m, 1.5H), 1.50-1.31 (m, 33H), 1.04-1.03 (m, 9H).

### S45-2: (cis)-di-tert-Butyl 1-(2-((1-(tert-butoxy)-2-methyl-1-oxopropan-2-yl)oxy)ethyl)-6-hydroxyhexahydropyrrolo[3,2-c]pyrazole-2,4-dicarboxylate

To a solution of S45-1 (4.5 g, 90% purity, 5.37 mmol) in tetrahydrofuran (25 mL) was added 1 M tetrabutylammonium fluoride in tetrahydrofuran (16 mL, 16 mmol) by dropwise and the reaction mixture was stirred at room temperature for 1 hour. The reaction mixture was concentrated to afford a crude product which was purified by silica gel column chromatography (petroleum ether:ethyl acetate=5:1 to 2:1) to give the title product (3.0 g, 90% purity from ¹H NMR, 97% of yield) as white solids. LC-MS (ESI): mass calcd. for C₂₅H₄₅N₃O₈ 515.3, m/z found 516.3 [M+H]⁺. ¹H NMR (400 MHz, CDCl₃) δ 4.55-4.46 (m, 1H), 4.30-4.13 (m, 2H), 3.89-3.87 (m, 1H), 3.68-3.84 (m, 4.5H), 3.05-3.00 (m, 1.5H), 1.48-1.43 (m, 27H), 1.39-1.38 (m, 6H).

### S45-3: (cis)-Di-tert-butyl 1-(2-((1-(tert-butoxy)-2-methyl-1-oxopropan-2-yl)oxy)ethyl)-6-((tert-butyldiphenylsilyl)oxy)hexahydropyrrolo[3,2-c]pyrazole-2,4-dicarboxylate

To a solution of S45-2 (3.00 g, 90% purity, 5.24 mmol) in dichloromethane (40 mL) was added Dess-Martin periodinane (8.88 g, 20.9 mmol) at 0° C. under nitrogen atmosphere. After stirred at room temperature for 3 hours, the reaction mixture was quenched with saturated sodium bicarbonate aqueous solution (150 mL) and extracted with dichloromethane (100 mL) for three times. The combined organic layers were washed with brine (50 mL) then dried Na₂SO₄₍ₛ₎, concentrated and purified by silica gel column chromatography (petroleum ether:ethyl acetate=10:1 to 3:1) to give the title product (2.0 g, 50% purity from ¹H NMR, 37% yield) as yellow oil. ¹H NMR (400 MHz, CDCl₃) δ 4.54-2.99 (m, 10H), 1.48-1.39 (m, 33H).

### S45-4: (cis)-Di-tert-butyl 1-(2-((1-(tert-butoxy)-2-methyl-1-oxopropan-2-yl)oxy)ethyl)-6,6-difluorohexahydropyrrolo[3,2-c]pyrazole-2,4-dicarboxylate

A solution of S45-3 (2.0 g, 50% purity, 1.95 mmol) in dichloromethane (40 mL) at −78° C. was added diethylaminosulfur trifluoride (1.5 mL, 11.4 mmol). The mixture was stirred at room temperature for 3 hours. Then it was quenched with saturated aqueous sodium bicarbonate solution (100 mL). The two layers were separated and the aqueous phase was extracted with dichloromethane (100 mL) twice. The combined organic extracts were washed with brine (100 mL), dried over Na₂SO₄₍ₛ₎, filtered and concentrated. The residue was purified by C18 column (acetonitrile:water (0.1% ammonium bicarbonate)=5% to 95%) to give the title compound (300 mg, 90% purity from ¹H NMR, 26% yield) as yellow oil. LC-MS (ESI): mass calcd. for C₂₅H₄₃F₂N₃O₇ 535.3, m/z found 536.3 [M+H]⁺. ¹H NMR (400 MHz, CDCl₃) δ 4.55-4.48 (m, 1H), 4.39-4.29 (m, 1H), 3.93-3.64 (m, 4H), 3.47-3.30 (m, 2H), 3.15-3.03 (m, 2H), 1.49-1.43 (m, 27H), 1.37 (s, 6H).

### S45-5: (cis)-tert-Butyl 1-(2-((1-(tert-butoxy)-2-methyl-1-oxopropan-2-yl)oxy)ethyl)-6,6-difluorohexahydropyrrolo[3,2-c]pyrazole-4(2H)-carboxylate

To a solution of S45-4 (300 mg, 90% purity, 0.504 mmol) in dichloromethane (30 mL) was added trifluoroacetic acid (1.5 mL) under nitrogen atmosphere. After stirred at 0° C. for 3 hours, the reaction mixture was added to the saturated aqueous sodium bicarbonate solution (10 mL). The two layers were separated and the aqueous phase was extracted with dichloromethane (10 mL) twice. The combined organic extracts were washed with brine (10 mL), dried over Na₂SO₄₍ₛ₎, filtered and concentrated to give the crude product (300 mg, 60% purity, 82% yield) as yellow oil. LC-MS (ESI): mass calcd. for C₂₀H₃₅F₂N₃O₅ 435.3, m/z found 436.3 [M+H]⁺.

### S45-6: (cis)-tert-Butyl 1-(2-((1-(tert-butoxy)-2-methyl-1-oxopropan-2-yl)oxy)ethyl)-6,6-difluoro-2-methylhexahydropyrrolo[3,2-c]pyrazole-4(2H)-carboxylate

To a solution of S45-5 (300 mg, 60% purity, 0.413 mmol) in methanol (3 mL) and acetic acid (0.3 mL) was added 37% of formaldehyde aqueous solution (0.6 mL, 8.06 mmol). The reaction was stirred at room temperature for 0.5 hour. Sodium cyanoborohydride (110 mg, 1.75 mmol) was added to the reaction mixture by pointwise. The reaction was stirred at room temperature for 1 hour. Water (10 mL) was added to the reaction mixture. The mixture was extracted with ethyl acetate (10 mL) twice. The combined organic phases were washed by saturated sodium bicarbonate aqueous solution (10 mL) and brine (10 mL), dried over Na₂SO₄₍ₛ₎, filtered and concentrated to give the crude product, which was purified by C18 column (acetonitrile:water (0.1% ammonium bicarbonate)=5% to 95%) to give the title compound (140 mg, 90% purity from ¹H NMR, 68% yield) as yellow oil. ¹H NMR (400 MHz, CDCl₃) δ 4.59-4.49 (m, 1H), 3.89-3.66 (m, 4H), 3.54-3.45 (m, 1H), 3.36-3.30 (m, 1H), 3.07-2.88 (m, 3H), 2.53 (s, 3H), 1.47-1.46 (m, 18H), 1.37 (s, 6H).

### S45: tert-Butyl 2-(2-((cis)-6,6-difluoro-2-methylhexahydropyrrolo[3,2-c]pyrazol-1(2H)-yl)ethoxy)-2-methylpropanoate

To a solution of S45-6 (130 mg, 90% purity, 0.260 mmol) in ethyl acetate (5 mL) was added 4.0 M hydrochloride in ethyl acetate (6 mL) under nitrogen atmosphere. After stirred at room temperature for 6 hours, the reaction mixture was added to the saturated aqueous sodium bicarbonate solution (10 mL). The two layers were separated and the aqueous phase was extracted with ethyl acetate (10 mL) twice. The combined organic extracts were washed with brine (10 mL), dried over Na₂SO₄₍ₛ₎, filtered and concentrated to give the title product (100 mg, 90% purity from ¹H NMR, 99% yield) as white solids. LC-MS (ESI): mass calcd. for C₁₆H₂₉F₂N₃O₃ 349.2, m/z found 350.2 [M+H]⁺. ¹H NMR (400 MHz, CDCl₃) δ 4.21-4.15 (m, 1H), 3.63-3.51 (m, 2H), 3.35-3.07 (m, 5H), 2.95-2.89 (m, 1H), 2.46 (s, 3H), 2.37-2.33 (m, 1H), 1.47 (s, 9H), 1.38 (s, 6H).

### Compound 99-A: ethyl (S)-6-(((cis)-1-(2-((1-(tert-butoxy)-2-methyl-1-oxopropan-2-yl)oxy)ethyl)-6,6-difluoro-2-methylhexahydropyrrolo[3,2-c]pyrazol-4(1H)-yl)methyl)-4-(3-fluoro-2-methylphenyl)-2-(thiazol-2-yl)-1,4-dihydropyrimidine-5-carboxylate

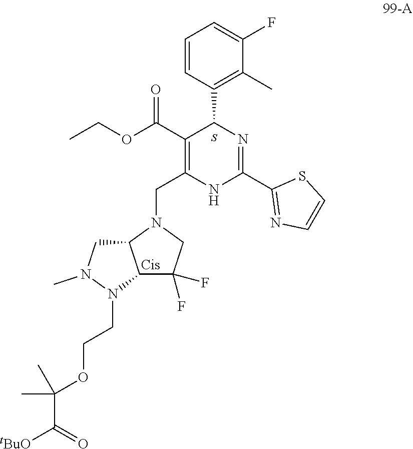

This is made from S45 and H2-1A according to typical method 1. LC-MS (ESI): mass calcd. for C₃₄H₄₅F₃N₆O₅S 706.3, m/z found 707.4 [M+H]⁺. ¹H NMR (400 MHz, CDCl₃) δ 9.16-9.08 (m, 1H), 7.84-7.85 (m, 1H), 7.47-7.41 (m, 1H), 7.10-6.94 (m, 3H), 6.03-5.97 (m, 1H), 4.29-3.92 (m, 6H), 3.61-2.78 (m, 8H), 2.54 (s, 3H), 1.48 (s, 9H), 1.39 (s, 6H), 1.29-1.23 (m, 3H), 1.14-1.09 (m, 3H).

Chiral separation by chiral Prep.HPLC (Column: Chiralpak IC 5 μm 20 mm*250 mm; Mobile Phase: Hex:IPA:DEA=90:10:0.2 at 20 mL/min; Temp: 30° C.; Wavelength: 254 nm) to afford the title compound 99-A (20 mg, 95% purity from ¹H NMR, 32% yield, 100% stereopure) and 99-B (20 mg, 95% purity from ¹H NMR, 32% yield, 99.0% stereopure) as yellow solids.

99-A: Chiral analysis (Column: Chiralpak IC 5 μm 4.6*250 mm; Mobile Phase: Hex:IPA:DEA=90:10:0.2 at 1.0 mL/min; Temp: 30° C.; Wavelength: 254 nm, R_{T}=6.998 min). ¹H NMR (400 MHz, CDCl₃) δ 9.16-9.08 (m, 1H), 7.84-7.85 (m, 1H), 7.47-7.41 (m, 1H), 7.10-6.94 (m, 3H), 6.03-5.97 (m, 1H), 4.29-3.92 (m, 6H), 3.61-2.78 (m, 8H), 2.54 (s, 3H), 1.48 (s, 9H), 1.39 (s, 6H), 1.29-1.23 (m, 3H), 1.14-1.09 (m, 3H).

99-B: Chiral analysis (Column: Chiralpak IC 5 μm 4.6*250 mm; Mobile Phase: Hex:IPA:DEA=90:10:0.2 at 1.0 mL/min; Temp: 30° C.; Wavelength: 254 nm, R_{T}=8.639 min). ¹H NMR (400 MHz, CDCl₃) δ 9.16-9.08 (m, 1H), 7.84-7.85 (m, 1H), 7.47-7.41 (m, 1H), 7.10-6.94 (m, 3H), 6.03-5.97 (m, 1H), 4.29-3.92 (m, 6H), 3.61-2.78 (m, 8H), 2.54 (s, 3H), 1.48 (s, 9H), 1.39 (s, 6H), 1.29-1.23 (m, 3H), 1.14-1.09 (m, 3H).

### Compound 99: 2-(2-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluoro-2-methylhexahydropyrrolo[3,2-c]pyrazol-1(2H)-yl)ethoxy)-2-methylpropanoic acid

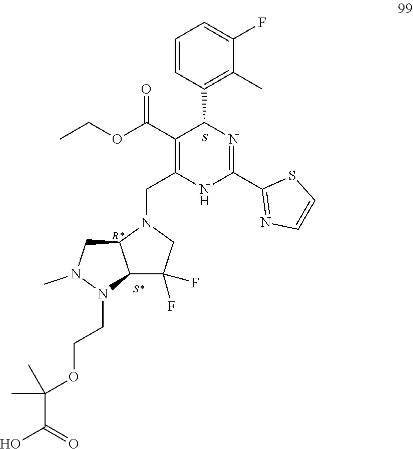

This is made from 99-A using typical method 3. LC-MS (ESI): mass calcd. for C₃₀H₃₇F₃N₆O₅S 650.3, m/z found 650.8 [M+H]⁺. ¹H NMR (400 MHz, CDCl₃) δ 9.14 (s, 1H), 7.81 (d, J=3.2 Hz, 1H), 7.41 (d, J=2.8 Hz, 1H), 7.10-7.00 (m, 1H), 6.98-6.95 (m, 1H), 6.91-6.88 (m, 1H), 6.00 (s, 1H), 4.31-4.19 (m, 3H), 4.09-3.98 (m, 2H), 3.74-3.61 (m, 2H), 3.45-3.15 (m, 4H), 3.12-3.04 (m, 2H), 2.87-2.76 (m, 1H), 2.68 (s, 3H), 2.54 (s, 3H), 1.46 (s, 3H), 1.40 (s, 3H), 1.12 (t, J=7.2 Hz, 3H).

### Preparation of Intermediate S61

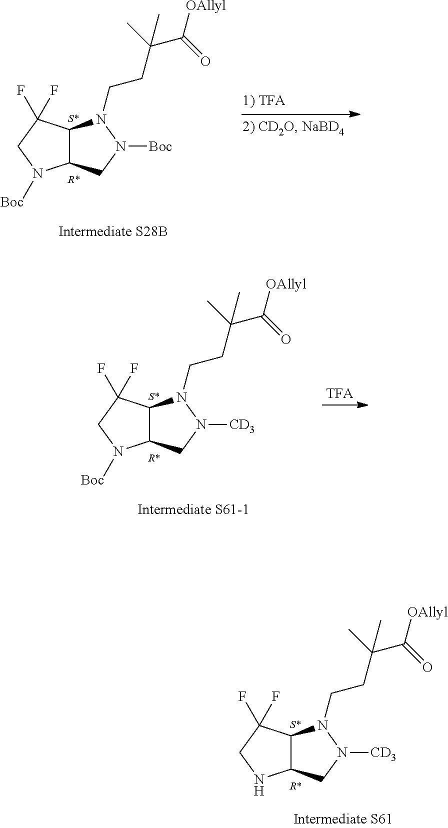

### S61-1: (cis)-tert-Butyl 1-(4-(allyloxy)-3,3-dimethyl-4-oxobutyl)-6,6-difluoro-2-methylhexahydropyrrolo[3,2-c]pyrazole-4(2H)-carboxylate-d₃

To a solution of S28B (100 mg, 90% purity, 0.179 mmol) in dichloromethane (40 mL) was dropwise added trifluoroacetic acid (2 mL) at 0° C. After stirred at 0° C. for 2.5 hours, the reaction mixture was poured into saturated sodium bicarbonate solution (50 mL). The organic layer was separated and the aqueous layer was extracted with dichloromethane (20 mL). The combined organic layers were washed with water (30 mL) twice, brine (30 mL) twice, dried over Na₂SO₄₍ₛ₎, filtered and concentrated to give a residue (50 mg) as colorless oil, which was diluted with methanol (5 mL) and the mixture was added acetic acid (0.2 mL) and 20% formaldehyde-d₂ in deuterium oxide (80 mg, 0.5 mmol). After stirred at room temperature for 0.5 hour, sodium borodeuteride (20 mg, 0.48 mmol) was added by point wise. The reaction was stirred at 0° C. for 0.5 hour. Water (10 mL) was added to the reaction mixture, the mixture was extracted with ethyl acetate (20 mL) twice. The combined organic phases were washed by saturated sodium bicarbonate aqueous solution (50 mL) and brine (30 mL). The organic layer was dried over Na₂SO₄₍ₛ₎, filtered and concentrated to give the crude product, which was purified by C18 column (acetonitrile:water (0.5% ammonium bicarbonate=5% to 80%) to give the title compound (50 mg, 90% purity from ¹H NMR, 60% yield) as yellow oil. ¹H NMR (400 MHz, CDCl₃) δ 5.96-5.86 (m, 1H), 5.34-5.21 (m, 2H), 4.58-4.56 (m, 2.5H), 4.51-4.46 (m, 0.5H), 3.92-3.71 (m, 2H), 3.40-3.28 (m, 2H), 3.05-3.02 (m, 0.5H), 2.85-2.64 (m, 2.5H), 1.86-1.80 (m, 2H), 1.46 (s, 9H), 1.22 (s, 6H).

### S61: Allyl 4-((cis)-6,6-difluoro-2-methylhexahydropyrrolo[3,2-c]pyrazol-1(2H)-yl)-2,2-dimethylbutanoate-d₃

To a solution of S61-1 (50 mg, 90% purity, 0.107 mmol) in dichloromethane (2 mL) was added trifluoroacetic acid (2 mL) at 0° C. After stirred at room temperature for 2 hours, the reaction mixture was concentrated under reduced pressure to give the crude product, which was purified by C18 column (acetonitrile:water (+0.5% ammonium bicarbonate)=20% to 70%) to give the title compound (35 mg, 90% purity from ¹H NMR, 92% yield) as brown oil. ¹H NMR (400 MHz, CDCl₃) δ 5.97-5.86 (m, 1H), 5.34-5.21 (m, 2H), 4.58-4.56 (m, 2H), 4.19-4.11 (m, 1H), 3.31-3.05 (m, 4H), 2.86-2.68 (m, 2H), 2.40-2.36 (m, 1H), 1.87-1.78 (m, 2H), 1.22 (s, 6H).

### Compound 100: 4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluoro-2-(methyl-d3)hexahydropyrrolo[3,2-c]pyrazol-1(2H)-yl)-2,2-dimethylbutanoic acid

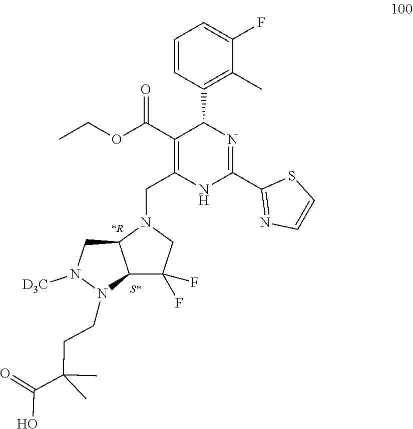 [SMILES: [2H]C([2H])([2H])N1C[C@@H]2[C@H](N1CCC(C)(C)C(=O)O)C(F)(F)CN2CC1=C(C(=O)OCC)[C@H](c2cccc(F)c2C)N=C(c2nccs2)N1]

This compound was made from S61 and H2-1A according to typical method 1 and 2 successively. LC-MS (ESI): mass calcd. for C₃₀H₃₄D₃F₃N₆O₄S 637.3, m/z found 637.8 [M+H]⁺. ¹H NMR (400 MHz, CDCl₃+ one drop of D₂O) δ 7.78 (d, J=3.2 Hz, 1H), 7.40 (d, J=3.6 Hz, 1H), 7.11-7.05 (m, 1H), 6.99-6.88 (m, 2H), 6.00 (s, 1H), 4.26 (d, J=16.4 Hz, 1H), 4.13 (d, J=17.2 Hz, 1H) 4.07-3.99 (m, 3H), 3.67-3.61 (m, 1H), 3.50-3.41 (m, 1H), 3.34-3.29 (m, 1H), 3.00-2.91 (m, 2H), 2.85-2.83 (m, 1H), 2.75-2.68 (m, 1H), 2.54 (s, 3H), 1.84-1.80 (m, 2H), 1.25 (s, 3H), 1.24 (s, 3H), 1.11 (t, J=7.2 Hz, 3H).

### Compound 101: 4-((cis)-4-((6-(3,4-Difluoro-2-methylphenyl)-5-(methoxycarbonyl)-2-(thiazol-2-yl)-3,6-dihydropyrimidin-4-yl)methyl)-6,6-difluoro-2-methylhexahydropyrrolo[3,2-c]pyrazol-1(2H)-yl)-2,2-dimethylbutanoic acid

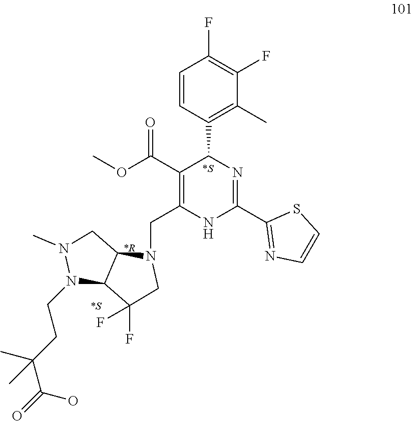 [SMILES: COC(=O)C1=C(CN2CC(F)(F)[C@@H]3[C@H]2CN(C)N3CCC(C)(C)C(=O)O)NC(c2nccs2)=N[C@H]1c1ccc(F)c(F)c1C]

This compound was made from S41 and H6-1B according to typical method 1 and typical method 2. LC-MS (ESI): mass calcd. for C₂₉H₃₄F₄N₆O₄S 638.2, m/z found 639.3 [M+H]⁺. ¹H NMR (400 MHz, CD₃OD) δ 9.20 (s, 1H), 7.79 (d, J=2.8 Hz, 1H), 7.41 (d, J=2.8 Hz, 1H), 6.95-6.85 (m, 2H), 5.93 (s, 1H), 4.28-4.09 (m, 3H), 3.72-3.64 (m, 1H), 3.60 (s, 3H), 3.50-3.35 (m, 2H), 3.05-2.94 (m, 2H), 2.87-2.79 (m, 2H), 2.73 (s, 3H), 2.58 (s, 3H), 1.82-1.78 (m, 2H), 1.24 (s, 3H), 1.23 (s, 3H).

### Compound 102: 4-((cis)-4-((6-(2-chloro-3-fluorophenyl)-5-(ethoxycarbonyl)-2-(thiazol-2-yl)-3,6-dihydropyrimidin-4-yl)methyl)-6,6-difluoro-2-methylhexahydropyrrolo[3,2-c]pyrazol-1(2H)-yl)-2,2-dimethylbutanoic acid

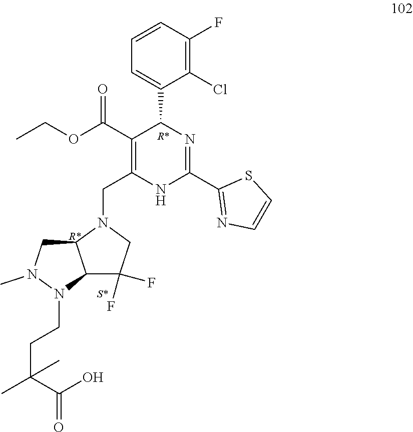 [SMILES: CCOC(=O)C1=C(CN2CC(F)(F)[C@@H]3[C@H]2CN(C)N3CCC(C)(C)C(=O)O)NC(c2nccs2)=N[C@H]1c1cccc(F)c1Cl]

This compound was made from H1-1A and S36 according to typical coupling method 1 and typical method 2 successively. Purified by C18 column (acetonitrile:water (+0.2% ammonium bicarbonate)=40%˜60%) to give the title compound (25.5 mg, 99.3% purity, 22.8% yield) as yellow solids. LC-MS (ESI): mass calcd. for C₂₉H₃₄ClF₃N₆O₄S 654.2, m/z found 655.3 [M+H]⁺. ¹H NMR (400 MHz, CDCl₃) δ 9.21 (s, 1H), 7.84 (d, J=3.2 Hz, 0.1H), 7.81 (d, J=2.8 Hz, 0.9H), 7.52 (d, J=3.2 Hz, 0.1H), 7.43 (d, J=3.2 Hz, 0.9H), 7.21-7.16 (m, 1H), 7.13-7.11 (m, 1H), 7.07-7.02 (m, 1H), 6.26 (s, 0.9H), 6.16 (d, J=2.4 Hz, 0.1H), 4.24 (d, J=17.2 Hz, 1H), 4.17-3.96 (m, 4H), 3.70-3.64 (m, 1H), 3.50-3.36 (m, 2H), 3.03-2.95 (m, 2H), 2.84-2.69 (m, 5H), 1.80 (t, J=8.0 Hz, 2H), 1.24 (s, 3H), 1.22 (s, 3H), 1.11 (t, J=7.2 Hz, 3H).

### Preparation of Intermediate S23

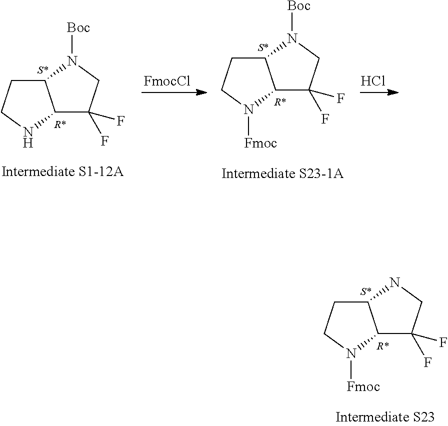

### S23-1: (cis)-4-((9H-Fluoren-9-yl)methyl) 1-tert-butyl 3,3-difluorotetrahydropyrrolo[3,2-b]pyrrole-1,4(2H,5H)-dicarboxylate

To a mixture of S1-12A (2.50 g, 90% purity, 9.1 mmol), sodium carbonate (4.50 g, 53.6 mmol) in tetrahydrofuran (50 mL) and water (15 mL) was added (9H-fluoren-9-yl)methyl carbonochloridate (2.60 g, 10.1 mmol). After stirred at 25° C. for 4 hours, the mixture was poured into water (20 mL) and extracted with ethyl acetate (40 mL) for three times. The combined organic layers were washed with brine (40 mL), dried over Na₂SO₄₍ₛ₎, filtered and concentrated to give a residue, which was purified by C18 (acetonitrile:water=10% to 90%) to give the desired compound (4.20 g, 99% yield) as white solids. LC-MS (ESI): mass calcd. for C₂₆H₂₈F₂N₂O₄ 470.2, m/z found 471.4 [M+H]⁺.

### S23: (cis)-(9H-Fluoren-9-yl)methyl 6,6-difluorohexahydropyrrolo[3,2-b]pyrrole-1(2H)-carboxylate

A solution of S23-1 (4.20 g, 8.93 mmol) in 5.0 M hydrochloride in ethyl acetate (20 mL) was stirred at room temperature for 2.5 hours. Then the mixture was concentrated to give a residue, which was basified with 2 M sodium bicarbonate aqueous solution to pH=9 and extracted with ethyl acetate (50 mL) for three times. The combined organic layers were washed with brine (50 mL), dried over Na₂SO₄₍ₛ₎, filtered and concentrated to afford the desired compound (3.30 g, 94% purity, 94% yield) as white solids. LC-MS (ESI): mass calcd. for C₂₁H₂₀F₂N₂O₂ 370.2, m/z found 371.4 [M+H]⁺.

### Compound 103-A: (9H-fluoren-9-yl)methyl (3aS,6aR)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrole-1(2H)-carboxylate

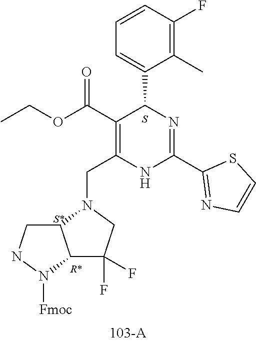

This compound was made from H2-1A with S23 according to Typical coupling method 1. Purified by C18 (acetonitrile:water=10% to 90%) to give the desired compound (5.50 g, 93% purity, 87% yield) as yellow oil. LC-MS (ESI): mass calcd. for C₃₉H₃₆F₃N₅O₄S 727.2, m/z found 728.5 [M+H]⁺.

### Compound 103: ethyl (S)-6-(((3aR,6aS)-3,3-difluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

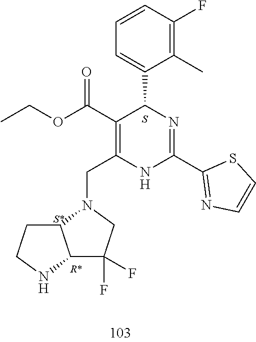

To a solution of compound 103-A (5.50 g, 93% purity, 7.03 mmol) in N,N-dimethylformide (15 mL) was added piperidine (3.00 g, 35.2 mmol). After stirred at room temperature for 3 hours, the mixture was purified by C18 (acetonitrile:water=10% to 90%) to give the desired compound (3.10 g, 81% yield, 93% purity) as a yellow solids. LC-MS (ESI): mass calcd. for C₂₄H₂₆F₃N₅O₂S 505.2, m/z found 506.4 [M+H]⁺. ¹H NMR (300 MHz, CDCl₃) δ 9.36 (s, 1H), 7.84 (d, J=3.0 Hz, 1H), 7.45 (d, J=3.3 Hz, 1H), 7.12-7.02 (m, 2H), 6.94 (t, J=9.0 Hz, 1H), 6.06 (s, 1H), 4.37 (d, J=8.7 Hz, 1H), 4.12-4.03 (m, 3H), 3.99-3.90 (m, 1H), 3.76 (t, J=6.0 Hz, 1H), 3.34-3.09 (m, 3H), 2.99-2.84 (m, 1H), 2.58-2.57 (m, 3H), 2.00-1.73 (m, 2H), 1.15 (t, J=7.2 Hz, 3H).

### Compound 104A and 104B: 4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)cyclohexane-1-carboxylic acid

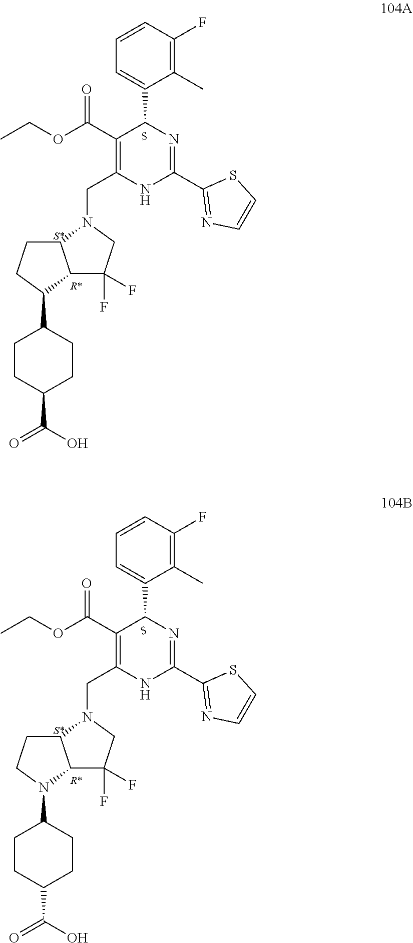

This compound was made from Compound 103 and tert-butyl 4-oxocyclohexane-1-carboxylate according to typical method 5 and typical method 3.

Compound 104A: LC-MS (ESI): mass calcd. for C₃₁H₃₆F₃N₅O₄S 631.2, m/z found 632.2 [M+H]⁺. ¹H NMR (400 MHz, DMSO-d6) δ 7.99 (s, 2H), 7.17-7.22 (m, 1H), 7.03-7.12 (m, 2H), 5.85 (br s, 1H), 4.12 (br s, 3H), 3.90-4.08 (m, 5H), 3.82-3.89 (m, 1H), 3.15-3.31 (m, 2H), 2.67 (br s, 1H), 2.54-2.62 (m, 2H), 2.39-2.47 (s, 3H), 1.87-2.15 (m, 5H), 1.54-1.73 (m, 1H), 1.42-1.53 (m, 3H), 1.05 (t, J=7.09 Hz, 3H).

Compound 104B: LC-MS (ESI): mass calcd. for C₃₁H₃₆F₃N₅O₄S 631.2, m/z found 632.2 [M+H]⁺. ¹H NMR (400 MHz, DMSO-d6) δ 7.99 (s, 2H), 7.19-7.21 (m, 1H), 7.02-7.12 (m, 2H), 5.85 (s, 1H), 4.12 (br s, 3H), 3.92-4.01 (m, 5H), 3.01-3.23 (m, 3H), 2.54-2.72 (m, 1H), 2.43 (s, 3H), 2.13-2.30 (m, 2H), 1.93-2.15 (m, 5H), 1.35-1.40 (m, 4H), 1.05 (t, J=7.09 Hz, 3H).

### Preparation of Intermediate S38A and Intermediate S38B

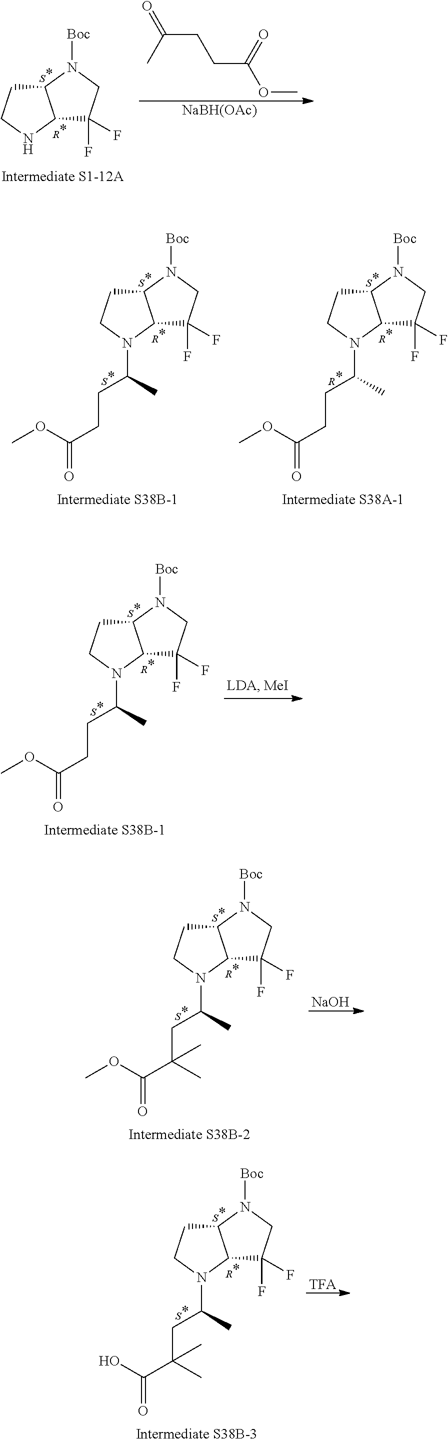

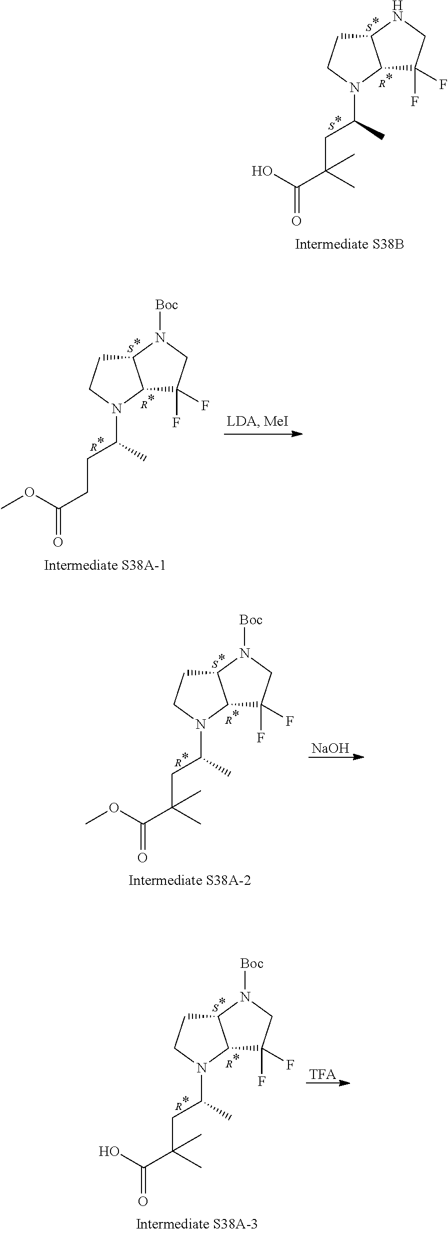

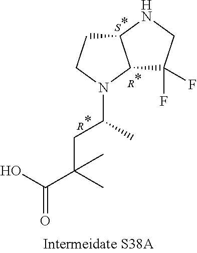 [SMILES: C[C@H](CC(C)(C)C(=O)O)N1CC[C@@H]2NCC(F)(F)[C@@H]21]

### S38B-1 and S38B-1: tert-butyl (cis)-3,3-difluoro-4-(5-methoxy-5-oxopentan-2-yl)hexahydropyrrolo[3,2-b]pyrrole-1(2H)-carboxylate

To a solution of S1-12A (500 mg, 2.014 mmol) in dichloromethane (20 mL) was added methyl 4-oxopentanoate (393 mg, 3.02 mmol), 1 M triisopropoxytitanium(IV) chloride in hexane (4.03 mL, 4.03 mmol) and acetic acid (0.1 mL). The mixture was stirred at room temperature for 10 minutes. Then sodium triacetoxyborohydride (2.13 g, 10.0 mmol) was added. The mixture was stirred at room temperature overnight. The mixture was diluted with dichloromethane (20 mL). The mixture was washed with saturated sodium bicarbonate aqueous solution (10 mL), dried over anhydrous sodium sulfate(s) and filtered. The filtrate was concentrated to give a residue which was purified by column chromatography on silica gel (petroleum ether:ethyl acetate=5:1) to give the desired two diastereomers (S38B-1: 199 mg, 12%; S38A-1: 432 mg, 27%, combined yield: 59%) as a yellow oil. LC-MS (ESI): mass calcd. for C₁₇H₂₈F₂N₂O₄ 362.2, m/z found 363.2 [M+H]⁺.

### S38B-2: tert-butyl (cis)-3,3-difluoro-4-(5-methoxy-4,4-dimethyl-5-oxopentan-2-yl)hexahydropyrrolo[3,2-b]pyrrole-1(2H)-carboxylate

To the solution of S38B-1 (432 mg, 1.192 mmol) in THE (10 mL) was added LDA (2 M in THF/heptane) (1.8 mL, 2 M, 3.57 mmol) at −78° C. The obtained mixture was stirred at −78° C. for 30 min, iodomethane (0.297 mL 4.77 mmol) was added to the mixture and the mixture was stirred at −78° C. for 1 hours and at room temperature for 2 hours.

aq. NH₄Cl was added to the mixture to quench and the mixture was extracted with EtOAc, the organic layer was dried and concentrated to give the crude which was react as starting material with the same procedure, and the final crude was purified on reversal column with acetonitrile and water (0.05% formic acid) to give the titled product (110 mg, 23%) as a colorless oil. LC-MS (ESI): mass calcd. for C₁₉H₃₂F₂N₂O₄ 390.2, m/z found 391.3 [M+H]⁺.

### S38B-3: 4-((cis)-4-(tert-butoxycarbonyl)-6,6-difluorohexahydropyrrolo[3,2-b]pyrrol-1(2H)-yl)-2,2-dimethylpentanoic acid

To the solution of S38B-2 (100 mg, 0.256 mmol) in MeOH (1 mL) and THF (1 mL) was added NaOH (0.427 mL, 3 M, 1.281 mmol). The mixture was stirred at 80° C. overnight. The mixture was cooled and neutralized with HCl (1 N) to pH=5, and the mixture was concentrated to dry, the residue was purified on reversal column with acetonitrile and water (0.05% formic acid) as a gradient eluent to give the titled product (72 mg, 74%) as a colorless oil. LC-MS (ESI): mass calcd. for C₁₈H₃₀F₂N₂O₄ 376.2, m/z found 377.3 [M+H]⁺.

### S38B: 4-((cis)-6,6-difluorohexahydropyrrolo[3,2-b]pyrrol-1(2H)-yl)-2,2-dimethylpentanoic acid

To the solution of S38B-3 (72 mg, 0.191 mmol) in DCM (4 mL, 1.326 g/mL, 62.45 mmol) was added TFA (4 mL, 1.49 g/mL, 52.27 mmol), and the mixture was stirred at room temperature for 1 hours. Then the mixture was concentrated to dry, and the residue was used in the next step directly. LC-MS (ESI): mass calcd. for C₁₃H₂₂F₂N₂O₂ 276.2, m/z found 277.2 [M+H]⁺.

S38A was made analogously.

### Compound 105A and 105B: 4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylpentanoic acid

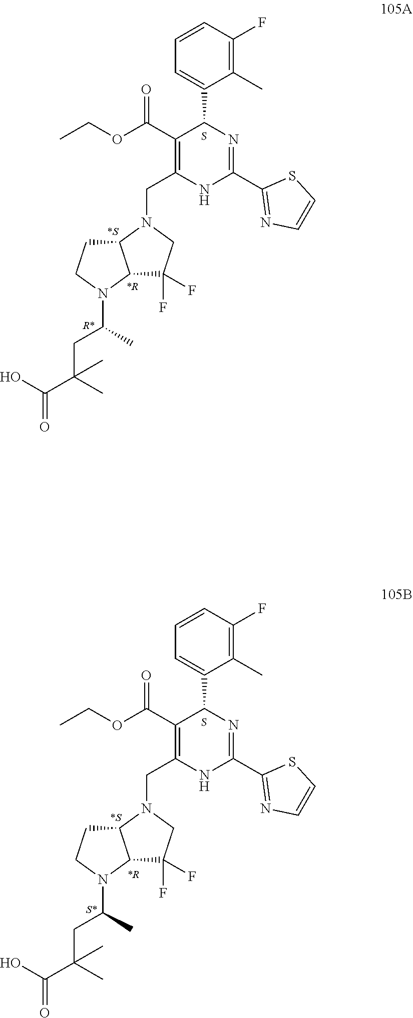

Compound 105A and 105B were made from H2-1A and S38A and S38B, respectively.

Compound 105A: LC-MS (ESI): mass calcd. for C₃₁H₃₈F₃N₅O₄S 633.2, m/z found 634.2 [M+H]⁺. 1H NMR (400 MHz, DMSO-d6) δ 9.41 (s, 1H), 7.92-8.01 (m, 2H), 7.14-7.24 (m, 1H), 7.01-7.09 (m, 2H), 5.87 (s, 1H), 4.04-4.24 (m, 2H), 3.76-4.01 (m, 3H), 2.99-3.27 (m, 5H), 2.90 (br s, 1H), 2.39-2.48 (s, 3H), 1.68-1.87 (m, 3H), 1.41-1.46 (m, 1H), 1.09-1.18 (m, 6H), 1.03-1.09 (m, 3H), 0.82-0.97 (m, 3H).

Compound 105B: LC-MS (ESI): mass calcd. for C₃₁H₃₈F₃N₅O₄S 633.2, m/z found 634.2 [M+H]⁺. 1H NMR (400 MHz, DMSO-d6) δ 9.42 (s, 1H), 7.96 (d, J=3.2 Hz, 1H), 7.93 (d, J=3.2 Hz, 1H), 7.11-7.29 (m, 1H), 7.01-7.11 (m, 2H), 5.87 (s, 1H), 4.12 (s, 2H), 3.97 (q, J=7.01 Hz, 2H), 3.80 (q, J=6.97 Hz, 1H), 3.42-3.63 (m, 1H), 3.12-3.28 (m, 2H), 2.84-3.11 (m, 2H), 2.55-2.72 (m, 1H), 2.44 (s, 3H), 1.66-1.87 (m, 3H), 1.44 (m, 1H), 0.98-1.19 (m, 12H).

### Preparation of Intermediate S16

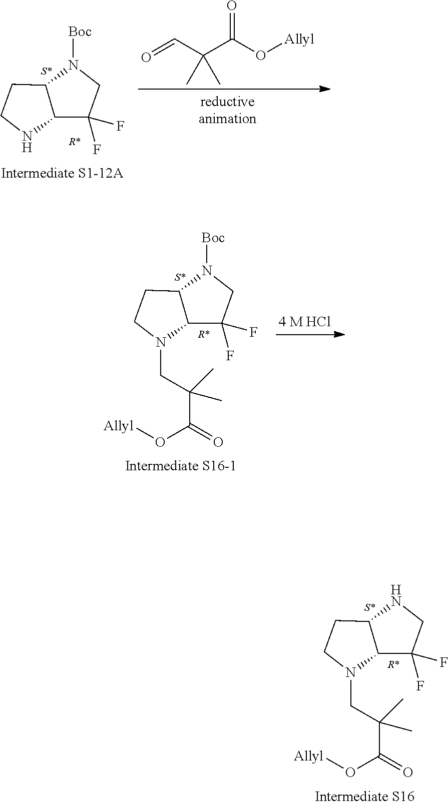

### S16-1: (cis)-tert-Butyl 4-(3-(allyloxy)-2,2-dimethyl-3-oxopropyl)-3,3-difluorohexahydropyrrolo[3,2-b]pyrrole-1(2H)-carboxylate

To a solution of S1-12A (100 mg, 90% purity, 0.363 mmol), allyl 2,2-dimethyl-3-oxopropanoate (200 mg, 90% purity, 1.15 mmol) in dichloromethane (5 mL) was added 1 M chlorotriisopropoxytitanium(IV) in dichloromethane (0.9 mL, 0.9 mmol) and acetic acid (26 mg, 0.433 mmol) at room temperature. After stirred at room temperature under nitrogen atmosphere for 2 hours, sodium triacetoxyborohydride (460 mg, 2.17 mmol) was added at room temperature overnight. The reaction mixture was filtered and the filtrate was concentrated under reduced pressure to give a residue, which was purified by silica gel column chromatography (petroleum ether:ethyl acetate=20:1) to give the title compound (80 mg, 90% purity from ¹H NMR, 51% yield) as light yellow oil. ¹H NMR (400 MHz, CDCl₃) δ 5.97-5.86 (m, 1H), 5.34-5.22 (m, 2H), 4.55 (d, J=8.4 Hz, 2H), 4.42-4.27 (m, 1H), 3.87-3.57 (m, 2H), 3.21-3.16 (m, 1H), 3.09-3.00 (m, 1H), 2.90-2.82 (m, 2H), 2.41-2.32 (m, 1H), 2.26-2.14 (m, 1H), 1.92-1.78 (m, 1H), 1.45 (s, 9H), 1.23 (s, 3H), 1.19 (s, 3H).

### S16: Allyl 3-((cis)-6,6-difluorohexahydropyrrolo[3,2-b]pyrrol-1(2H)-yl)-2,2-dimethylpropanoate hydrochloride

A solution of S16-1 (80 mg, 90% purity, 0.185 mmol) in 4 M hydrochloride in ethyl acetate (5 mL, 20 mmol) was stirred at 25° C. for 1 hour. The reaction mixture was concentrated to give the title compound (55 mg, 76% purity, 69% yield) as white solids. LC-MS (ESI): mass calcd. for C₁₄H₂₂F₂N₂O₂ 288.12, m/z found 289.5 [M+H]⁺.

### Compound 106-A: ethyl 4-(3-acetoxy-2-methylphenyl)-6-(((3aR,6aS)-4-(3-(allyloxy)-2,2-dimethyl-3-oxopropyl)-3,3-difluorohexahydropyrrolo[3,2-b]pyrrol-1(2H)-yl)methyl)-2-(thiazol-2-yl)-1,4-dihydropyrimidine-5-carboxylate

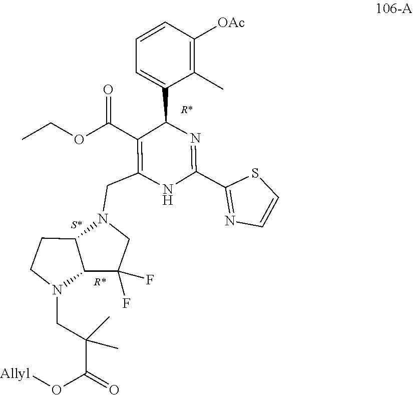

This compound was prepared from H21-1A and S16 according to typical coupling method 1. Purified by C18 (acetonitrile:water=10% to 70%) to give the title compound (70 mg, 100% purity, 45% yield) as yellow solids. LC-MS (ESI): mass calcd. for C₃₄H₄₁F₂N₅O₆S 685.3, m/z found 686.7 [M+H]⁺.

### Compound 106-B: ethyl 6-(((cis)-4-(3-(allyloxy)-2,2-dimethyl-3-oxopropyl)-3,3-difluorohexahydropyrrolo[3,2-b]pyrrol-1(2H)-yl)methyl)-4-(3-hydroxy-2-methylphenyl)-2-(thiazol-2-yl)-1,4-dihydropyrimidine-5-carboxylate

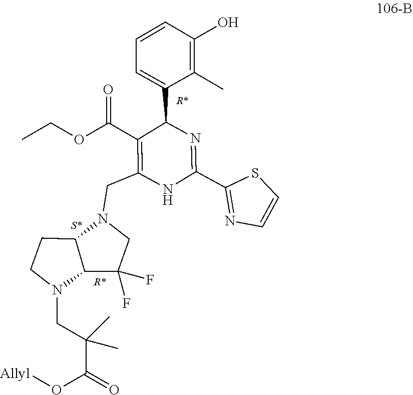

Compound 106-B was made from 106-A under treatment of K₂CO₃ in MeOH. Purified by C18 column (acetonitrile:water=30% to 80%) to give the title compound (50 mg, 90% purity from ¹H NMR, 76% yield) as yellow solids. ¹HNMR (400 MHz, CDCl₃) δ 7.83 (d, J=3.2 Hz, 1H), 7.39 (d, J=3.2 Hz, 1H), 6.97 (t, J=8.0 Hz, 1H), 6.81 (d, J=7.2 Hz, 1H), 6.65 (d, J=8.0 Hz, 1H), 6.02 (s, 1H), 6.98-5.88 (m, 1H), 5.35-5.30 (m, 1H), 5.24-5.21 (m, 1H), 4.86 (s, 1H), 4.56 (d, J=6.8 Hz, 1H), 4.25-4.21 (m, 1H), 4.09-3.98 (m, 3H), 3.73-3.67 (m, 1H), 3.54-3.45 (m, 1H), 3.29-3.20 (m, 1H), 3.17-3.11 (m, 1H), 3.05-3.02 (m, 1H), 2.94-2.81 (m, 2H), 2.62-2.57 (m, 1H), 2.52 (s, 3H), 1.88-1.80 (m, 2H), 1.25-1.21 (m, 6H), 7.83 (d, J=3.2 Hz, 1H), 1.18 (t, J=6.8 Hz, 3H).

### Compound 106: (cis)-3-(4-((5-(ethoxycarbonyl)-6-(3-hydroxy-2-methylphenyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylpropanoic acid (Single Diastereomer)

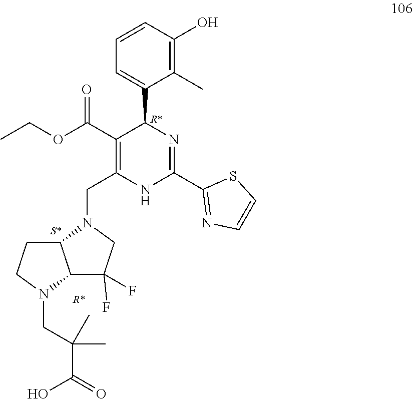 [SMILES: CCOC(=O)C1=C(CN2CC(F)(F)[C@H]3[C@@H]2CCN3CC(C)(C)C(=O)O)NC(c2nccs2)=N[C@@H]1c1cccc(O)c1C]

this compound was made from 106-B using condition in typical method 2. Purified by C18 column (acetonitrile:water=30% to 80%) to give the title compound (19 mg, 99% purity, 45% yield) as yellow solids. LC-MS (ESI): mass calcd. for C₂₉H₃₅F₂N₅O₅S 603.2, m/z found 604.3 [M+H]⁺. ¹H NMR (400 MHz, DMSO-d₆) δ 12.14 (s, 1H), 9.39 (s, 0.1H), 9.29-9.22 (m, 1.9H), 7.98-7.95 (m, 1H), 7.90-7.89 (m, 1H), 6.93-6.89 (m, 0.9H), 6.82-6.79 (m, 0.1H), 6.68-6.62 (m, 2H), 5.84 (s, 0.8H), 5.74 (s, 0.2H), 4.17-3.91 (m, 4H), 3.81-3.70 (m, 1H), 3.56-3.47 (m, 1H), 3.22-3.13 (m, 1H), 3.05-2.89 (m, 3H), 2.76-2.59 (m, 2H), 2.34-2.28 (m, 3H), 1.81-1.73 (m, 2H), 1.12-1.04 (m, 9H).

### Compound 107: 4-(4-((5-(ethoxycarbonyl)-6-(3-fluoro-2-methylphenyl)-2-(5-methyloxazol-4-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

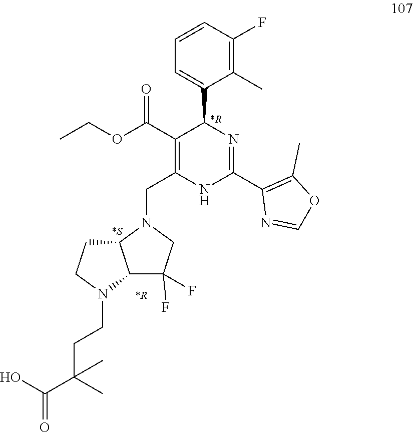 [SMILES: CCOC(=O)C1=C(CN2CC(F)(F)[C@H]3[C@@H]2CCN3CCC(C)(C)C(=O)O)NC(c2ncoc2C)=N[C@@H]1c1cccc(F)c1C]

This compound was made from S9 and H15-1A according to typical coupling method land 3 successively. Purified by C18 column (acetonitrile:water (+0.02% ammonium bicarbonate)=5% to 95%) to give the title compound (35 mg, 96.6% purity, 79% yield) as yellow solids. LC-MS (ESI): mass calcd. for C₃₁H₃₈F₃N₅O₅ 617.3, m/z found 618.3 [M+H]⁺. ¹H NMR (400 MHz, CDCl₃) δ 8.02 (s, 1H), 7.17-7.12 (m, 1H), 7.05-7.03 (m, 1H), 6.96-6.92 (m, 1H), 5.97 (s, 1H), 4.26 (d, J=16.8 Hz, 1H), 4.15 (d, J=16.8 Hz, 1H), 4.08 (q, J=7.2 Hz, 2H), 3.93-3.88 (m, 1H), 3.39-3.37 (m, 1H), 3.34-3.24 (m, 2H), 3.08-3.00 (m, 1H), 2.90-2.83 (m, 1H), 2.64-2.62 (m, 1H), 2.57-2.47 (m, 7H), 2.02-1.92 (m, 2H), 1.83 (t, J=8.0 Hz, 2H), 1.23 (s, 6H), 1.15 (t, J=7.2 Hz, 3H).

### Preparation of Intermediate S17

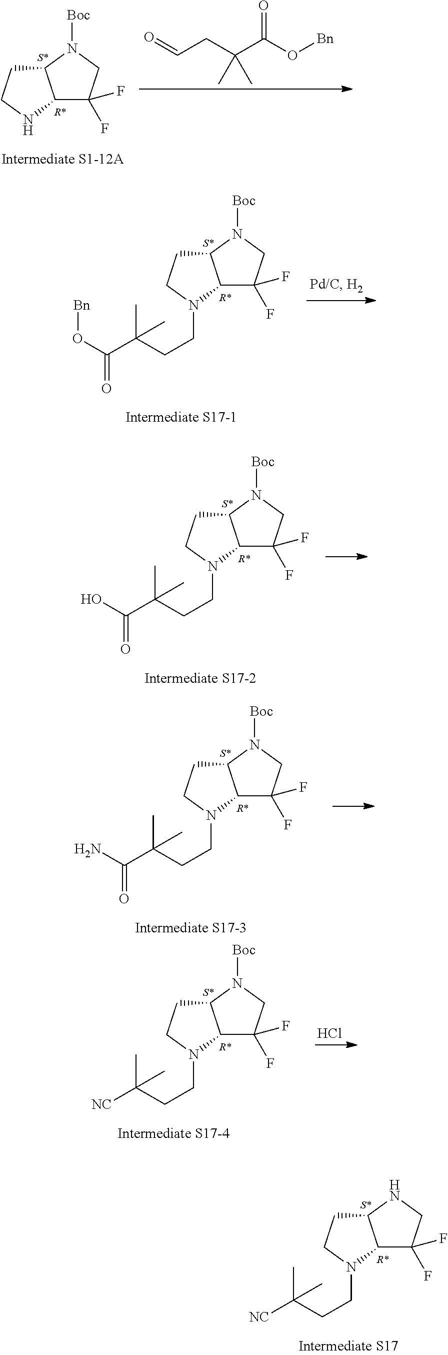

### S17-1: (cis)-tert-Butyl 4-(4-(benzyloxy)-3,3-dimethyl-4-oxobutyl)-3,3-difluorohexahydropyrrolo[3,2-b]pyrrole-1(2H)-carboxylate

A solution of S1-12A (100 mg, 90% purity, 0.363 mmol) and benzyl 2,2-dimethyl-4-oxobutanoate (170 mg, 95% purity, 0.733 mmol) in methanol (10 mL) was adjusted pH to 5 with acetic acid and then the mixture was stirred at room temperature for 2 hours. Then, sodium cyanoborohydride (120 mg, 1.91 mmol) was added at 0° C. and the mixture was stirred at room temperature overnight. Water (15 mL) was added and the mixture was extracted with ethyl acetate (15 mL) twice. The combined organic layers were washed with water (30 mL), brine (30 mL), dried over Na₂SO₄₍ₛ₎ and filtered. The filtrate was concentrated and purified by C18 column (acetonitrile:water (+0.1% ammonium bicarbonate)=65% to 85%) to give the title compound (110 mg, 97% purity, 65% yield) as colourless oil. LC-MS (ESI): mass calcd. for C₂₄H₃₄F₂N₂O₄ 452.2, m/z found 453.5 [M+H]⁺.

### S17-2: 4-((cis)-4-(tert-Butoxycarbonyl)-6,6-difluorohexahydropyrrolo[3,2-b]pyrrol-1(2H)-yl)-2,2-dimethylbutanoic acid

To the solution of S17-1 (560 mg, 95% purity, 1.18 mmol) in ethanol (15 mL) was added 10% palladium on charcoal wt. (200 mg, 0.188 mmol). The mixture was stirred at room temperature under hydrogen atmosphere (balloon) for 3 hours. Then the mixture was filtered and the filtrate was concentrated to give the title compound (600 mg, 46% purity, 65% yield) as colourless oil. LC-MS (ESI): mass calcd. for C₁₇H₂₈F₂N₂O₄ 362.2, m/z found 361.1 [M−H]⁺.

### S17-3: (cis)-tert-Butyl 4-(4-amino-3,3-dimethyl-4-oxobutyl)-3,3-difluorohexahydropyrrolo[3,2-b]pyrrole-1(2H)-carboxylate

To a solution of S17-3 (600 mg, 46% purity, 0.762 mmol) in ethyl acetate (30 mL) was added di(1H-imidazol-1-yl)methanone (150 mg, 0.925 mmol) at room temperature. The solution was stirred at room temperature under nitrogen atmosphere for 1 hour. Then, 28% ammonia in water (200 mg, 1.60 mmol) was added. After stirred at room temperature under nitrogen atmosphere for 3 hours, the mixture was taken up into water (30 mL). The aqueous layer was separated and extracted with ethyl acetate (20 mL) twice. The combined organic layers were washed with water (30 mL) and brine (30 mL), dried over Na₂SO₄₍ₛ₎ and filtered. The filtrate was concentrated under reduced pressure to give the title compound (230 mg, 95% purity from ¹H NMR, 79% yield) as white solids. ¹H NMR (400 MHz, CDCl₃) δ 5.80 (s, 1H), 5.50 (s, 1H), 4.50-4.35 (m, 1H), 3.91-3.75 (m, 1H), 3.67-3.56 (m, 1H), 3.29-3.25 (m, 1H), 3.07-2.92 (m, 2H), 2.40-2.20 (m, 3H), 1.83-1.73 (m, 3H), 1.46 (s, 9H), 1.22 (s, 6H).

### S17-4: (cis)-tert-Butyl 4-(3-cyano-3-methylbutyl)-3,3-difluorohexahydropyrrolo[3,2-b]pyrrole-1(2H)-carboxylate

To a solution of S17-3 (230 mg, 95% purity, 0.605 mml) in dichloromethane (5 mL) was added pyridine (100 mg, 1.26 mml) and trifluoroacetic anhydride (200 mg, 0.952 mml) at 0° C. After stirred at room temperature for 2 hours, the reaction mixture was quenched with water (10 mL) and extracted with ethyl acetate (10 mL) for three times. The combined organic layers were washed with brine (10 mL), dried over Na₂SO₄₍ₛ₎ and filtered. The filtrate was concentrated under reduced pressure to give the title compound (200 mg, 60% purity from ¹H NMR, 58% yield) as yellow oil. ¹H NMR (400 MHz, CDCl₃) δ 4.52-4.37 (m, 1H), 3.92-3.59 (m, 2H), 3.29-3.26 (m, 1H), 3.16-2.93 (m, 2H), 2.57-2.50 (m, 1H), 2.38-2.20 (m, 2H), 1.81-1.70 (m, 3H), 1.46 (s, 9H), 1.37 (s, 6H).

### S17: 4-((cis)-6,6-Difluorohexahydropyrrolo[3,2-b]pyrrol-1(2H)-yl)-2,2-dimethylbutanenitrile hydrochloride

To a solution of S17-4 (200 mg, 60% purity, 0.349 mmol) in ethyl acetate (2 mL) was added dropwise of 3.5 M hydrochloride in ethyl acetate (2 mL, 7.0 mmol). After stirred at room temperature overnight, the mixture was concentrated to give the title compound (160 mg, 60% purity from ¹H NMR, 98% yield) as white solids. ¹H NMR (400 MHz, DMSO-d₆) δ 4.52-4.37 (m, 1H), 3.92-3.59 (m, 2H), 3.29-3.26 (m, 1H), 3.16-2.93 (m, 2H), 2.57-2.50 (m, 1H), 2.38-2.20 (m, 2H), 1.81-1.70 (m, 3H), 1.46 (s, 9H), 1.37 (s, 6H).

### Compound 108-A: ethyl (S)-6-(((cis)-4-(3-cyano-3-methylbutyl)-3,3-difluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

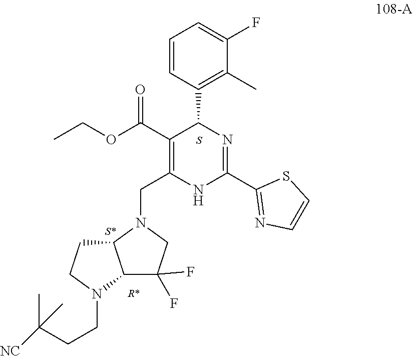

This compound was made from H2-1A with S17 according to Typical coupling method 1. Purified by C18 column (acetonitrile:water (+0.1% ammonium bicarbonate)=75% to 85%) to give the title compound (180 mg, 98% purity, 68% yield) as yellow solids. LC-MS (ESI): mass calcd. for C₃₀H₃₅F₃N₆O₂S 600.2, m/z found 601.5 [M+H]⁺.

### Compound 108: ethyl (S)-6-(((cis)-3,3-difluoro-4-(3-methyl-3-(1H-tetrazol-5-yl)butyl)hexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate (Single Diastereomer)

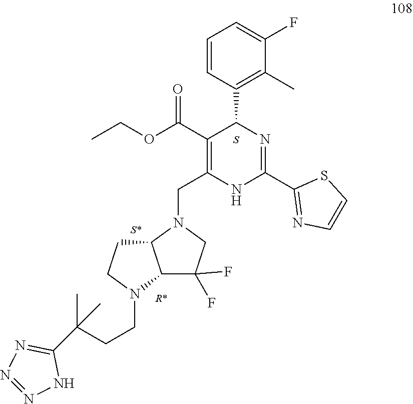

To a solution of compound 108-A (60 mg, 98% purity, 0.098 mmol) in 1,4-dioxane (1.5 mL) was added azidotrimethylsilane (120 mg, 1.04 mmol) and dibutylstannanone (15 mg, 0.06 mmol) at room temperature under nitrogen atmosphere. After stirred at 140° C. for 10 hours in a microwave reactor, the mixture was concentrated under reduced pressure to afford a residue, which was purified by C18 column (acetonitrile:water=30% to 70%) to give the title compound (10.1 mg, 98.4% purity, 16% yield) as yellow solids. LC-MS (ESI): mass calcd. for C₃₀H₃₆F₃N₉O₂S 643.3, m/z found 644.3 [M+H]⁺. ¹H NMR (400 MHz, CD₃OD) δ 7.80 (d, J=3.2 Hz, 1H), 7.61 (d, J=2.8 Hz, 1H), 7.06-6.97 (m, 2H), 6.85-6.81 (m, 1H), 5.85 (s, 1H), 4.11 (d, J=16.8 Hz, 1H), 4.01 (d, J=16.8 Hz, 1H), 3.94 (q, J=7.2 Hz, 2H), 3.77-3.72 (m, 1H), 3.14-3.09 (m, 2H), 2.96-2.91 (m, 1H), 2.74-2.69 (m, 1H), 2.39 (s, 3H), 2.49-2.33 (m, 1H), 1.98-1.87 (m, 3H), 1.77-1.73 (m, 1H), 1.38 (s, 6H), 1.22 (t, J=7.6 Hz, 3H), 1.02 (t, J=7.2 Hz, 2H).

### Compound 109: ethyl (S)-6-(((cis)-4-(2,2-dimethyl-3-(methylsulfonamido)-3-oxopropyl)-3,3-difluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate (Single Diastereomer)

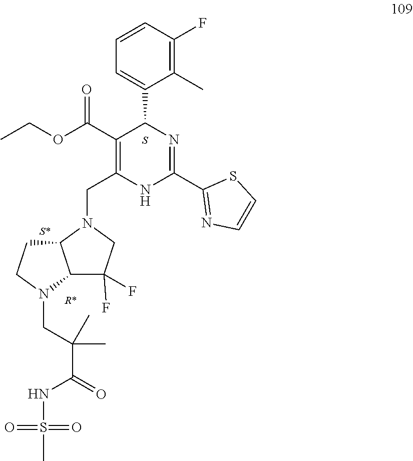

To a solution of compound 1A (160 mg, 90% purity, 0.238 mmol) and N-(3-dimethylaminopropyl)-N-ethylcarbodiimide hydrochloride (120 mg, 0.626 mmol), N,N-dimethylpyridin-4-amine (128 mg, 1.05 mmol) in dichloromethane (10 mL) was added methanesulfonamide (68 mg, 0.715 mmol). After stirred at 40° C. for 16 hours, the reaction mixture diluted with water (10 mL) and extracted with dichloromethane (10 mL) for three times. The combined organic phases were washed with brine (10 mL), dried over Na₂SO₄₍ₛ₎ and filtered. The filtrate was concentrated to give a residue, which was purified by C18 (acetonitrile:water=30% to 80%) to give the title compound (56.8 mg, 96.7% purity, 34% yield) as yellow solids. LC-MS (ESI): mass calcd. for C₃₀H₃₇F₃N₆O₅S₂ 682.8, m/z found 683.3 [M+H]⁺. ¹H NMR (400 MHz, DMSO-d₆) δ 11.55 (s, 1H), 9.39 (s, 1H), 8.00-7.96 (m, 1H), 7.92-7.91 (m, 1H), 7.21-7.14 (m, 1H), 7.06-6.99 (m, 2H), 5.88 (s, 0.8H), 5.76 (s, 0.2H), 4.20-4.06 (m, 2H), 4.00-3.94 (m, 2H), 3.85-3.73 (m, 1H), 3.64-3.54 (m, 1H), 3.25-3.15 (m, 4H), 3.11-2.91 (m, 3H), 2.87-2.82 (m, 1H), 2.71-2.62 (m, 1H), 2.44 (s, 3H), 1.83-1.74 (m, 2H), 1.15-1.06 (m, 6H), 1.04 (t, J=7.2 Hz, 3H).

### Preparation of Intermediate S18

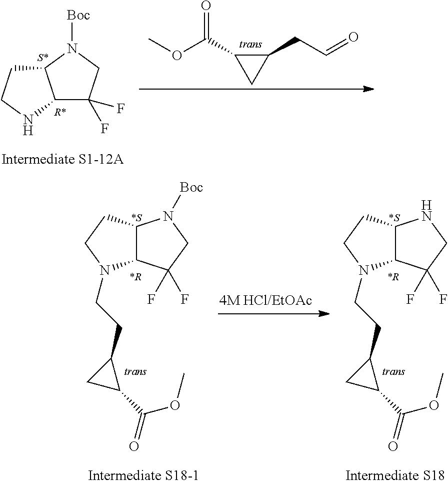

### S18-1: (cis)-tert-Butyl 3,3-difluoro-4-(2-((trans)-2-(methoxycarbonyl)cyclopropyl)ethyl)hexahydropyrrolo[3,2-b]pyrrole-1(2H)-carboxylate

To a solution of S1-12A (300 mg, 90% purity, 1.09 mmol) and (trans)-methyl 2-(2-oxoethyl)cyclopropanecarboxylate (260 mg, 90% purity, 1.65 mmol) in methanol (7 mL) was added acetic acid (0.3 mL) at room temperature. After stirred at room temperature under nitrogen atmosphere for 1 hour, sodium cyanoborohydride (430 mg, 6.84 mmol) was added. After stirred at room temperature for 3 hours, the reaction mixture was filtered and the filtrate was concentrated under reduced pressure to give a residue, which was diluted with water (50 mL), extracted with ethyl acetate (50 mL) twice. The combined organic layers were dried over Na₂SO₄₍ₛ₎ and filtered. The filtrate was concentrated to give a residue, which was purified by C18 column (acetonitrile:water=40% to 50%) to give the title compound (215 mg, 90% purity from ¹H NMR, 48% yield) as yellow oil. ¹H NMR (400 MHz, CDCl₃) δ 4.50-4.32 (m, 1H), 3.90-3.79 (m, 1H), 3.67-3.60 (m, 3H), 3.46-3.35 (m, 0.6H), 3.27-3.18 (m, 0.4H), 3.06-2.96 (m, 2H), 2.70-2.66 (m, 0.7H), 2.47-2.10 (m, 2.3H), 1.90-1.78 (m, 1H), 1.45-1.38 (m, 11H), 1.22-1.13 (m, 1H), 1.10 (d, J=6.8 Hz, 1H), 1.01 (d, J=6.4 Hz, 1H), 0.85-0.67 (m, 2H).

### S18: methyl (trans)-2-(2-(6,6-difluorohexahydropyrrolo[3,2-b]pyrrol-1(2H)-yl)ethyl)cyclopropane-1-carboxylate

The solution of S18-1 (150 mg, 90% purity, 0.361 mmol) in 4 M hydrochloride in ethyl acetate (4 mL, 20.0 mmol) was stirred at room temperature under nitrogen atmosphere for 1 hour. The mixture was concentrated under reduced pressure to give crude product which was used in next step directly.

### Compound 110-A: ethyl (S)-6-(((cis)-3,3-difluoro-4-(2-((trans)-2-(methoxycarbonyl)cyclopropyl)ethyl)hexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

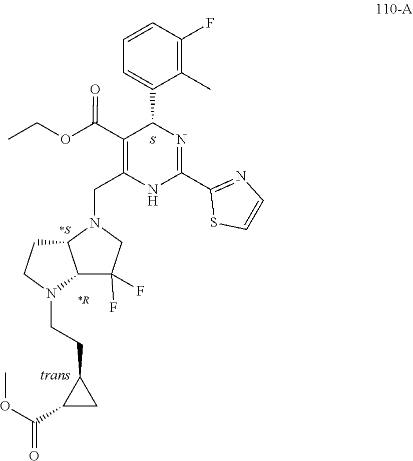

This is made from H2-1A with S18 according to Typical coupling method 1. Purified by C18 column (acetonitrile:water=60% to 70%) to give the title compound (70 mg, 90% purity from ¹H NMR, 30% yield) as light yellow solids. LC-MS (ESI): mass calcd. for C₃₁H₃₆F₃N₅O₄S, 631.7, m/z found 632.6 [M+H]⁺. ¹H NMR (400 MHz, CDCl₃) δ 9.35 (s, 1H), 7.83 (d, J=3.2 Hz, 1H), 7.40-7.38 (m, 1H), 7.11-7.04 (m, 1H), 7.01-6.98 (m, 1H), 6.92-6.88 (m, 1H), 6.00 (s, 1H), 4.23 (d, J=17.2 Hz, 1H), 4.14 (d, J=17.6 Hz, 1H), 4.04-3.99 (m, 2H), 3.90-3.80 (m, 1H), 3.67 (s, 1.2H), 3.66 (s, 1.8H), 3.35-3.17 (m, 3H), 3.02-2.85 (m, 2H), 2.67-2.61 (m, 1H), 2.54 (s, 1.2H), 2.53 (s, 1.8H), 2.44-2.38 (m, 1H), 1.97-1.92 (m, 2H), 1.46-1.40 (m, 2H), 1.25-1.20 (m, 2H), 1.11 (t, J=7.2 Hz, 3H), 0.91-0.82 (m, 1H), 0.79-0.71 (m, 1H).

### Compound 110: (trans)-2-(2-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)ethyl)cyclopropane-1-carboxylic acid

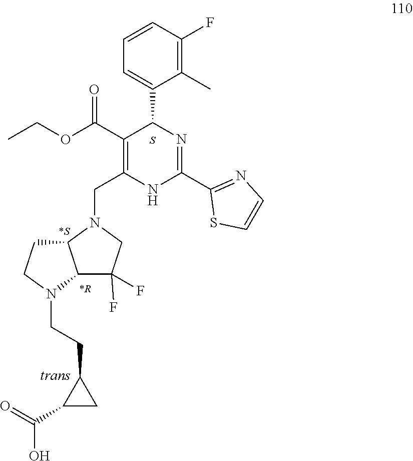

Typical method 4: To a solution of compound 110-A (45 mg, 90% purity, 0.064 mmol) in tetrahydrofuran (0.9 mL), methanol (0.3 mL) and water (0.3 mL) was added lithium hydroxide monohydrate (8.5 mg, 0.203 mmol) at 0° C. After stirred at 0° C. for 2 hours, the mixture was diluted with water (10 mL), and acidified with 1 M hydrochloride aqueous solution to pH 5˜6. The aqueous layer was extracted with ethyl acetate (20 mL) for three times. The combined organic layers were washed with brine (20 mL), dried over Na₂SO₄₍ₛ₎ and filtered. The filtrate was concentrated to give a residue, which was purified by Prep. HPLC (Column: Waters Xbridge C18 (5 μm 19*150 mm), Mobile Phase A: water (+0.1% ammonium bicarbonate), Mobile Phase B: acetonitrile, UV: 254 nm, Flow rate: 15 mL/min, Gradient: 30-60% (% B)), to give the title compound (5.1 mg, 92.2% purity, 12% yield) as yellow solids. LC-MS (ESI): mass calcd. for C₃₀H₃₄F₃N₅O₄S, 617.6, m/z found 618.3 [M+H]⁺. ¹H NMR (400 MHz, CD₃OD) δ 7.93 (d, J=3.2 Hz, 1H), 7.73 (d, J=3.2 Hz, 1H), 7.21-7.10 (m, 2H), 6.98-6.93 (m, 1H), 5.98 (s, 1H), 4.26 (d, J=16.8 Hz, 1H), 4.16 (d, J=16.8 Hz, 1H), 4.09 (q, J=6.8 Hz, 2H), 3.97-3.88 (m, 1H), 3.29-3.27 (m, 2H), 3.11-3.02 (m, 1H), 2.96-2.88 (m, 1H), 2.74-2.65 (m, 1H), 2.53 (s, 3H), 2.52-2.45 (m, 1H), 2.09-1.95 (m, 2H), 1.68-1.55 (m, 1.6H), 1.50-1.33 (m, 3.4H), 1.17-1.09 (m, 4H), 0.83-0.75 (m, 1H).

### Preparation of Intermediate S19

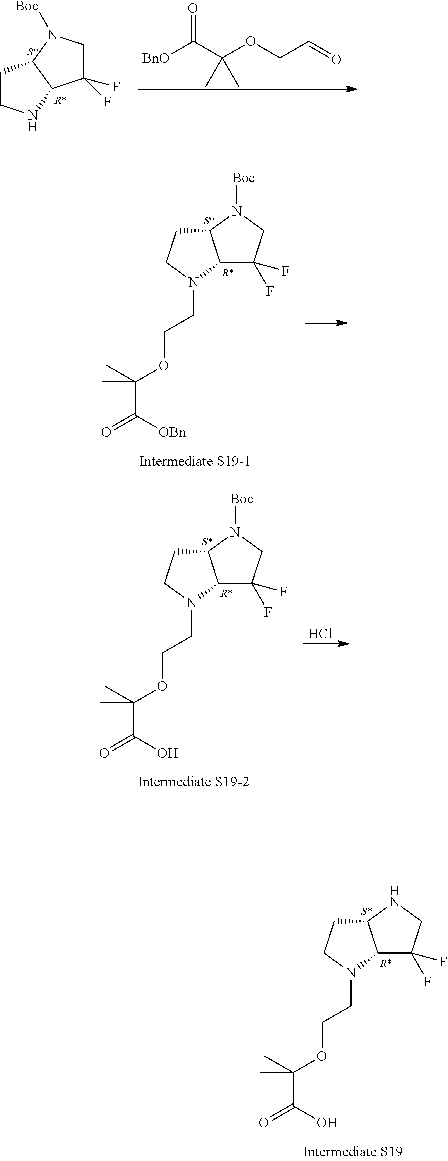 [SMILES: CC(C)(C)OC(=O)N1CC(F)(F)[C@@H]2NCC[C@@H]21 ; CC(C)(OCC=O)C(=O)OCc1ccccc1 ; CC(C)(C)OC(=O)N1CC(F)(F)[C@H]2[C@@H]1CCN2CCOC(C)(C)C(=O)OCc1ccccc1 ; CC(C)(C)OC(=O)N1CC(F)(F)[C@H]2[C@@H]1CCN2CCOC(C)(C)C(=O)O ; Cl ; CC(C)(OCCN1CC[C@@H]2NCC(F)(F)[C@@H]21)C(=O)O]

### S19-1: (cis)-tert-Butyl 4-(2-((1-(benzyloxy)-2-methyl-1-oxopropan-2-yl)oxy)ethyl)-3,3-difluorohexahydropyrrolo[3,2-b]pyrrole-1(2H)-carboxylate

The solution of S1-12A (588 mg, 90% purity, 2.13 mmol), benzyl 2-methyl-2-(2-oxoethoxy)propanoate (600 mg, 70% purity, 1.78 mmol) and acetic acid (226 mg, 3.76 mmol) in dichloromethane (10 mL) was stirred at 25° C. for 20 minutes. Then sodium triacetoxyborohydride (1.88 g, 8.87 mmol) was added in portions and the mixture was stirred at 25° C. for another 3 hours. The reaction mixture was adjusted to pH=8˜9 with saturated sodium bicarbonate aqueous solution (20 mL). The organic layer was separated and the aqueous layer was extracted with dichloromethane (10 mL) for three times. The combined organic phases were washed with brine (10 mL), dried over Na₂SO₄₍ₛ₎ and filtered. The filtrate was concentrated to give a residue, which was purified by C-18 (acetonitrile:water=50% to 70%) to give the title compound (250 mg, 90% purity from ¹H NMR, 27% yield) as colorless oil. LC-MS (ESI): mass calcd. for C₂₄H₃₄F₂N₂O₅ 468.2, m/z found 469.6 [M+H]⁺. ¹H NMR (400 MHz, CDCl₃) δ 7.38-7.30 (m, 5H), 5.18 (d, J=12.0 Hz, 1H), 5.14 (d, J=12.4 Hz, 1H), 4.46-4.41 (m, 0.5H), 4.36-4.31 (m, 0.5H), 3.89-3.73 (m, 1H), 3.67-3.55 (m, 1H), 3.51-3.48 (m, 2H), 3.25-3.14 (m, 2H), 3.07-3.01 (m, 1H), 2.67-2.60 (m, 1H), 2.44-2.37 (m, 1H), 2.29-2.14 (m, 1H), 1.85-1.69 (m, 2H), 1.46 (s, 4.5H), 1.45 (s, 4.5H), 1.43 (s, 6H).

### S19-2: 2-(2-((cis)-4-(tert-Butoxycarbonyl)-6,6-difluorohexahydropyrrolo[3,2-b]pyrrol-1(2H)-yl)ethoxy)-2-methylpropanoic acid

To a mixture of S19-1 (200 mg, 90% purity, 0.384 mmol) in methanol (5 mL) was added 20% palladium hydroxide on activated carbon (30 mg, 0.043 mmol) under nitrogen atmosphere. After stirred at 50° C. under hydrogen atmosphere (H₂ balloon) for 3 hours, the mixture was filtered and the filtrate was concentrated to give the title compound (230 mg, 40% purity, 63% yield) as colorless oil. LC-MS (ESI): mass calcd. for C₁₇H₂₈F₂N₂O₅ 378.2, m/z found 379.2 [M+H]⁺.

### S19: 2-(2-((cis)-6,6-Difluorohexahydropyrrolo[3,2-b]pyrrol-1(2H)-yl)ethoxy)-2-methylpropanoic acid hydrochloride

A solution of S19-2 (230 mg, 40% purity, 0.243 mmol) in 4 M hydrochloride in ethyl acetate (3 mL) was stirred at 25° C. for 1 hour. The reaction mixture was concentrated to give the title compound (85 mg, 82% purity, 91% yield) as white solids. LC-MS (ESI): R_{T}=0.294 min, mass calcd. for C₁₂H₂₀F₂N₂O₃.HCl 314.1, m/z found 279.1 [M−HCl+H]⁺.

### Compound 111: 2-(2-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)ethoxy)-2-methylpropanoic acid (Single Diastereomer)

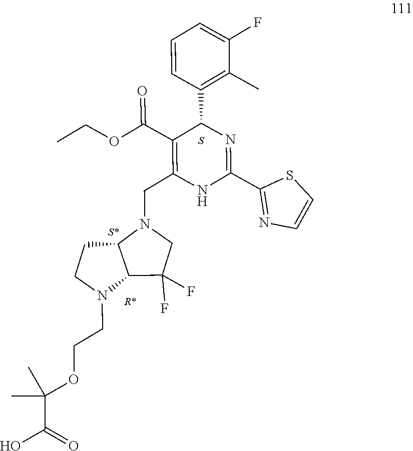

This compound was made from H2-1A with S19 according to Typical coupling method 1. Purified by Prep. HPLC (Column: Waters Xbridge C18 (5 μm 19*150 mm), Mobile Phase A: water (0.1% trifluoroacetic acid), Mobile Phase B: acetonitrile, UV: 214 nm, Flow rate: 15 mL/min, Gradient: 15-50% (% B)) and further purified by C-18 (acetonitrile:water (0.1% sodium bicarbonate)=10% to 40%) to give the title compound (39.4 mg, 99.2% purity, 28.4% yield) as yellow solids. LC-MS (ESI): R_{T}=3.461 min, mass calcd. for C₃₀H₃₆F₃N₅O₅S 635.2, m/z found 636.2 [M+H]⁺. ¹H NMR (400 MHz, CD₃OD) δ 7.80 (d, J=3.2 Hz, 1H), 7.60 (d, J=3.2 Hz, 1H), 7.07-6.99 (m, 2H), 6.85-6.80 (m, 1H), 5.86 (s, 1H), 4.16 (d, J=16.4 Hz, 1H), 4.03 (d, J=16.4 Hz, 1H), 3.95 (q, J=7.2 Hz, 2H), 3.86-3.81 (m, 1H), 3.63-3.49 (m, 3H), 3.31-3.23 (m, 2H), 3.04-2.86 (m, 3H), 2.77-2.71 (m, 1H), 2.40 (s, 1.5H), 2.39 (s, 1.5H), 2.00-1.90 (m, 2H), 1.33 (s, 3H), 1.32 (s, 3H), 1.02 (t, J=7.2 Hz, 3H).

### Preparation of Intermediate S21

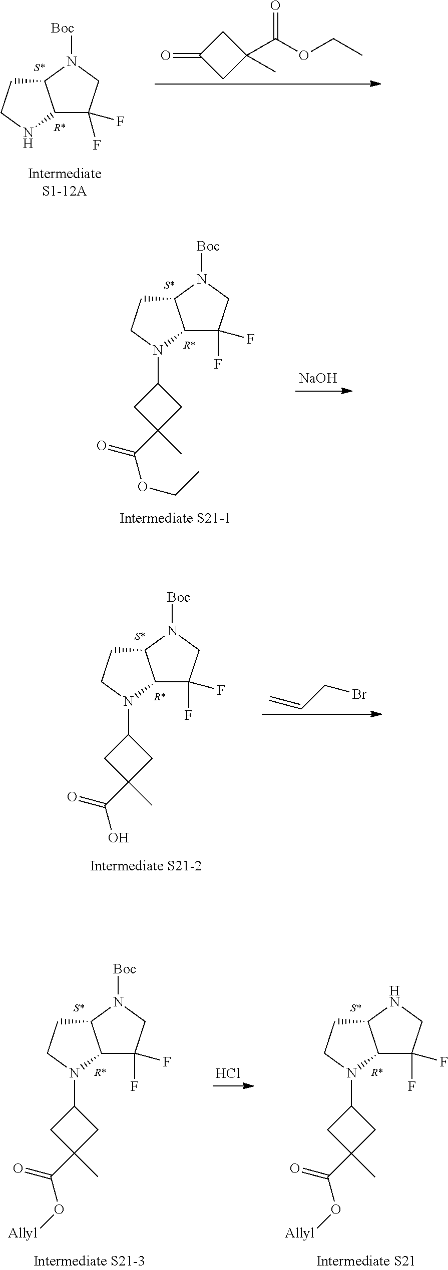

### S21-1: (cis)-tert-Butyl 4-(3-(ethoxycarbonyl)-3-methylcyclobutyl)-3,3-difluorohexahydropyrrolo[3,2-b]pyrrole-1(2H)-carboxylate

To a mixture of S1-12A (500 mg, 90% purity, 1.81 mmol) in dichloromethane (10 mL) was added acetic acid (0.5 mL), ethyl 1-methyl-3-oxocyclobutanecarboxylate (420 mg, 2.69 mmol) and 1 M triisopropoxytitanium(IV) chloride in dichloromethane (3.5 mL, 3.5 mmol). The mixture was stirred at 25° C. for 1 hour, then sodium triacetoxyborohydride (1.92 g, 9.06 mmol) was added. After stirred at 25° C. for 16 hours, the reaction mixture was quenched with saturated sodium bicarbonate aqueous solution (30 mL) and filtered. The filtrate was extracted with dichloromethane (20 mL) for three times. The combined organic phases were washed with brine (20 mL), dried over Na₂SO₄₍ₛ₎ and filtered. The filtrate was concentrated to give a residue, which was purified by C-18 (acetonitrile:water=20% to 60%) to give the title compound (570 mg, 90% purity from ¹H NMR, 73% yield) as colorless oil. LC-MS (ESI): mass calcd. for C₁₉H₃₀F₂N₂O₄ 388.2, m/z found 389.2 [M+H]⁺. ¹H NMR (400 MHz, CDCl₃) δ 4.53-4.49 (m, 0.5H), 4.43-4.39 (m, 0.5H), 4.19-4.11 (m, 2H), 3.90-3.75 (m, 1H), 3.66-3.47 (m, 1.5H), 3.42-3.19 (m, 1.5H), 3.06-3.00 (m, 1H), 2.70-2.54 (m, 2H), 2.48-2.41 (m, 1H), 2.17-1.85 (m, 4H), 1.46 (s, 9H), 1.39 (s, 1.5H), 1.37 (s, 1.5H), 1.29-1.24 (m, 3H).

### S21-2: 3-((cis)-4-(tert-Butoxycarbonyl)-6,6-difluorohexahydropyrrolo[3,2-b]pyrrol-1(2H)-yl)-1-methylcyclobutanecarboxylic acid

To a solution of S21-1 (470 mg, 90% purity, 1.09 mmol) in tetrahydrofuran (4 mL), methanol (4 mL) and water (2 mL) was added sodium hydroxide (152 mg, 3.8 mmol). After stirred at 25° C. for 4 hours, the reaction mixture was diluted with water (20 mL), and acidified to pH=6˜7 with 1 M hydrochloride aqueous solution. The aqueous layer was extracted with ethyl acetate (10 mL) for three times. The combined organic layers were washed with brine (10 mL), dried over Na₂SO₄₍ₛ₎ and filtered. The filtrate was concentrated to give the title compound (420 mg, 90% purity from ¹H NMR, 96% yield) as yellow solids. ¹H NMR (400 MHz, CDCl₃) δ 4.54-4.39 (m, 1H), 3.89-3.76 (m, 1H), 3.69-3.45 (m, 2H), 3.30-3.22 (m, 1H), 3.11-3.01 (m, 1H), 2.76-2.58 (m, 2H), 2.52-2.47 (m, 1H), 2.17-1.87 (m, 4H), 1.46 (s, 9H), 1.42 (s, 1.5H), 1.41 (s, 1.5H).

### S21-3: (cis)-tert-Butyl 4-(3-((allyloxy)carbonyl)-3-methylcyclobutyl)-3,3-difluorohexahydropyrrolo[3,2-b]pyrrole-1(2H)-carboxylate

To a solution of S21-2 (420 mg, 90% purity, 0.979 mmol) in N,N-dimethylformamide (5 mL) was added potassium carbonate (273 mg, 1.98 mmol) and allyl bromide (180 mg, 1.49 mmol). After stirred at 25° C. for 16 hours, the reaction mixture was diluted with water (20 mL), and extracted with ethyl acetate (10 mL) for three times. The combined organic phases were washed with brine (10 mL), dried over Na₂SO₄₍ₛ₎ and filtered. The filtrate was concentrated to give a residue, which was purified by C-18 (acetonitrile:water=30% to 70%) to give the title compound (420 mg, 90% purity from ¹H NMR, 96% yield) as colorless oil. LC-MS (ESI): mass calcd. for C₂₀H₃₀F₂N₂O₄ 400.2, m/z found 401.2 [M+H]⁺. ¹H NMR (400 MHz, CDCl₃) δ 5.98-5.87 (m, 1H), 5.35-5.33 (m, 0.4H), 5.31-5.29 (m, 0.6H), 5.25-5.22 (m, 1H), 4.62-4.58 (m, 2H), 4.53-4.49 (m, 0.6H), 4.44-4.39 (m, 0.4H), 3.90-3.75 (m, 1H), 3.66-3.19 (m, 3H), 3.07-2.99 (m, 1H), 2.70-2.55 (m, 2H), 2.50-2.44 (m, 1H), 2.19-1.78 (m, 4H), 1.46 (s, 9H), 1.42 (s, 1.8H), 1.40 (s, 1.2H).

### S21: Allyl 3-((cis)-6,6-difluorohexahydropyrrolo[3,2-b]pyrrol-1(2H)-yl)-1-methylcyclobutanecarboxylate hydrochloride

A solution of S21-3 (420 mg, 90% purity, 0.944 mmol) in 4 M hydrochloride in ethyl acetate (5 mL) was stirred at 25° C. for 1 hour. The reaction mixture was concentrated to give the title compound (380 mg, 75% purity, 90% yield) as white solids. LC-MS (ESI): mass calcd. for C₁₅H₂₂F₂N₂O₂ 300.2, m/z found 301.1 [M+H]⁺.

### Compound 112-M: ethyl (S)-6-(((cis)-4-(3-((allyloxy)carbonyl)-3-methylcyclobutyl)-3,3-difluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

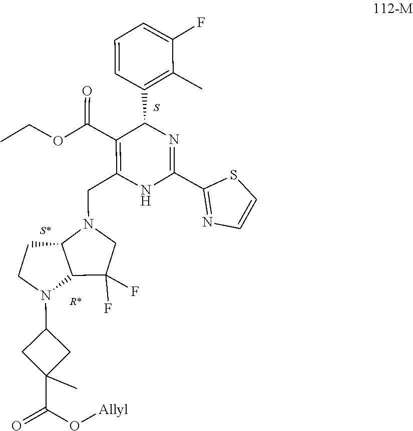

This is made from H2-1A with S21 according to Typical coupling method 1. Purified by C-18 (acetonitrile:water=10% to 70%) to give the title compound (480 mg, 90% purity from ¹H NMR, 77.6% yield) as yellow solids. LC-MS (ESI): mass calcd. for C₃₃H₃₇F₃N₅O₄S 657.3, m/z found 658.6 [M+H]⁺. ¹H NMR (400 MHz, CDCl₃) δ 9.32 (s, 0.6H), 9.31 (s, 0.4H), 7.82 (d, J=3.2 Hz, 1H), 7.40-7.39 (m, 1H), 7.10-7.04 (m, 1H), 6.99 (d, J=7.2 Hz, 1H), 6.92-6.88 (m, 1H), 6.00 (s, 1H), 5.98-5.88 (m, 1H), 5.36-5.34 (m, 0.4H), 5.31-5.30 (m, 0.6H), 5.26-5.22 (m, 1H), 4.63-4.59 (m, 2H), 4.24 (d, J=17.6 Hz, 1H), 4.11-3.99 (m, 3H), 3.83-3.78 (m, 1H), 3.59-3.21 (m 3H), 3.17-3.10 (m, 1H), 3.00-2.92 (m, 1H), 2.70-2.47 (m, 6H), 2.08-1.86 (m, 4H), 1.44 (s, 1.8H), 1.42 (s, 1.2H), 1.11 (t, J=7.2 Hz, 3H).

Compound 112-M was separated by chiral Prep. HPLC (Column: Chiralpak IG 5 μm 20*250 mm; Mobile Phase: Hex:EtOH=75:25 at 18 mL/min; Temp: 30° C.; Wavelength: 214 nm) to give the title compounds 112-A (160 mg, 90% purity from ¹H NMR, 40% yield) and 112-B (200 mg, 90% purity from ¹H NMR, 50% yield) as yellow oil.

112-A: LC-MS (ESI): mass calcd. for C₃₃H₃₇F₃N₅O₄S 657.3, m/z found 658.2 [M+H]⁺. Chiral analysis (Column: Chiralpak IG 5 μm 4.6*250 mm; Mobile Phase: Hex:EtOH=75:25 at 1 mL/min; Temp: 30° C.; Wavelength: 254 nm; R_{T}=8.478 min). ¹H NMR (400 MHz, CDCl₃) δ 9.30 (s, 1H), 7.83 (d, J=2.8 Hz, 1H), 7.39 (d, J=2.8 Hz, 1H), 7.09-7.04 (m, 1H), 6.99 (d, J=7.6 Hz, 1H), 6.92-6.87 (m, 1H), 6.00 (s, 1H), 5.97-5.89 (m, 1H), 5.35-5.23 (m, 2H), 4.62-4.61 (m, 2H), 4.24 (d, J=17.6 Hz, 1H), 4.10-3.97 (m, 3H), 3.83-3.78 (m, 1H), 3.45-3.12 (m, 4H), 3.00-2.93 (m, 1H), 2.65-2.54 (m, 6H), 2.03-1.81 (m, 4H), 1.42 (s, 3H), 1.11 (t, J=6.8 Hz, 3H).

112-B: LC-MS (ESI): mass calcd. for C₃₃H₃₇F₃N₅O₄S 657.3, m/z found 658.2 [M+H]⁺. Chiral analysis (Column: Chiralpak IG 5 μm 4.6*250 mm; Mobile Phase: Hex:EtOH=75:25 at 1 mL/min; Temp: 30° C.; Wavelength: 254 nm; R_{T}=6.991 min). ¹H NMR (400 MHz, CDCl₃) δ 9.32 (s, 1H), 7.82 (d, J=2.8 Hz, 1H), 7.39 (d, J=3.2 Hz, 1H), 7.10-7.04 (m, 1H), 6.99 (d, J=7.6 Hz, 1H), 6.92-6.88 (m, 1H), 6.00 (s, 1H), 5.98-5.88 (m, 1H), 5.32 (d, J=18.4 Hz, 1H), 5.24 (d, J=11.6 Hz, 1H), 4.60-4.59 (m, 2H), 4.24 (d, J=17.6 Hz, 1H), 4.11-3.97 (m, 3H), 3.84-3.79 (m, 1H), 3.61-3.53 (m, 1H), 3.48-3.42 (m, 1H), 3.30-3.21 (m, 1H), 3.16-3.10 (m, 1H), 3.00-2.92 (m, 1H), 2.69-2.63 (m, 1H), 2.54-2.48 (m, 5H), 2.08-2.04 (m, 2H), 1.95-1.88 (m, 2H), 1.44 (s, 3H), 1.11 (t, J=7.2 Hz, 3H).

### Compound 112: 3-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)-1-methylcyclobutane-1-carboxylic acid

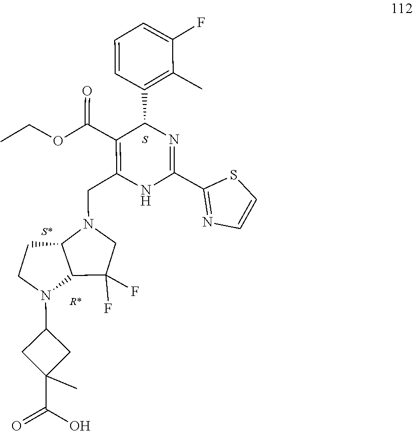

This is made according to typical method 2 from compound 112-M. Purified by Pre. HPLC (Column: Xbridge C18 (5 μm 19*150 mm), Mobile Phase A: water (+0.1% ammonium bicarbonate), Mobile Phase B: acetonitrile, UV: 254 nm, Flow rate: 15 mL/min, Gradient: 20-55% (% B)) to give the title compound (23.5 mg, 97.3% purity, 45.1% yield) as yellow solids. LC-MS (ESI): mass calcd. for C₃₀H₃₄F₃N₅O₄S 617.2, m/z found 617.9 [M+H]⁺. ¹H NMR (400 MHz, CD₃OD) δ 7.93-7.92 (m, 1H), 7.73 (d, J=3.2 Hz, 1H), 7.19-7.11 (m, 2H), 6.98-6.93 (m, 1H), 5.99 (s, 1H), 4.29 (d, J=17.2 Hz, 1H), 4.15-4.05 (m, 3H), 3.93-3.86 (m, 1H), 3.57-3.40 (m, 2H), 3.31-3.26 (m, 1H), 3.17-3.00 (m, 2H), 2.75-2.45 (m, 6H), 2.09-1.90 (m, 4H), 1.43 (s, 1.5H), 1.41 (s, 1.5H), 1.15 (t, J=7.2 Hz, 3H).

### Compound 112A: (trans)-3-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)-1-methylcyclobutane-1-carboxylic acid

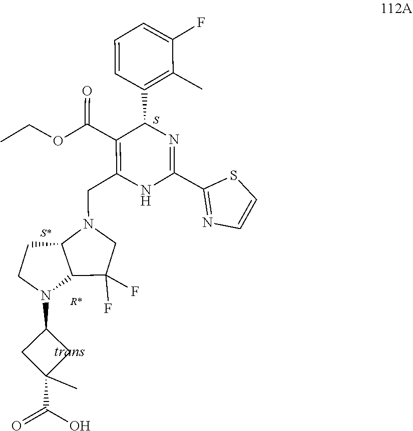

This compound was made from 112-A using typical method 2. purified by Pre. HPLC (Column: Xbridge C18 (5 μm 19*150 mm), Mobile Phase A: water (+0.1% ammonium bicarbonate), Mobile Phase B: acetonitrile, UV: 254 nm, Flow rate: 15 mL/min, Gradient: 25-50% (% B)) to give the title compound (71.5 mg, 97.3% purity, 58.8% yield) as yellow solids. LC-MS (ESI): mass calcd. for C₃₀H₃₄F₃N₅O₄S 617.2, m/z found 618.2 [M+H]⁺. ¹H NMR (400 MHz, CD₃OD) δ 7.89 (d, J=3.2 Hz, 1H), 7.71-7.70 (m, 1H), 7.16-7.08 (m, 2H), 6.95-6.90 (m, 1H), 5.96 (s, 1H), 4.25 (d, J=16.8 Hz, 1H), 4.11-4.02 (m, 3H), 3.88-3.83 (m, 1H), 3.46-3.35 (m, 2H), 3.28-3.23 (m, 1H), 3.15-3.10 (m, 1H), 3.06-2.98 (m, 1H), 2.66-2.54 (m, 3H), 2.50 (s, 3H), 1.98-1.85 (m, 4H), 1.38 (s, 3H), 1.12 (t, J=7.2 Hz, 3H).

### Compound 112B: (cis)-3-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)-1-methylcyclobutane-1-carboxylic acid

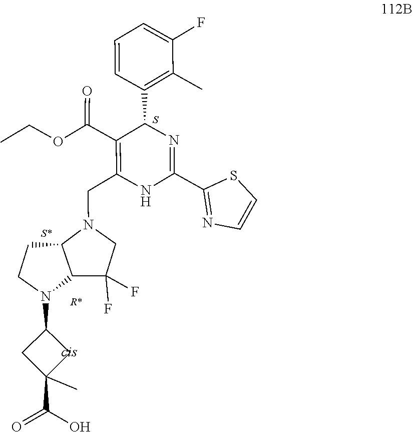

This compound was made from 112-B using typical method 2. Purified by Pre. HPLC (Column: Xbridge C18 (5 μm 19*150 mm), Mobile Phase A: water (+0.1% ammonium bicarbonate), Mobile Phase B: acetonitrile, UV: 254 nm, Flow rate: 15 mL/min, Gradient: 30-45% (% B)) to give the title compound (78.2 mg, 95.8% purity, 49.2% yield) as yellow solids. LC-MS (ESI): mass calcd. for C₃₀H₃₄F₃N₅O₄S 617.2, m/z found 618.3 [M+H]⁺. ¹H NMR (400 MHz, CD₃OD) δ 7.90-7.89 (m, 1H), 7.71-7.69 (m, 1H), 7.16-7.08 (m, 2H), 6.95-6.90 (m, 1H), 5.96 (s, 1H), 4.26 (d, J=16.8 Hz, 1H), 4.12-4.10 (m, 3H), 3.89-3.84 (m, 1H), 3.53-3.44 (m, 2H), 3.29-3.23 (m, 1H), 3.16-3.10 (m, 1H), 3.06-2.98 (m, 1H), 2.72-2.66 (m, 1H), 2.50 (s, 3H), 2.47-2.42 (m, 2H), 2.06-2.02 (m, 2H), 1.96-1.88 (m, 2H), 1.40 (s, 3H) 1.12 (t, J=7.2 Hz, 3H).

### Preparation of Intermediate S22

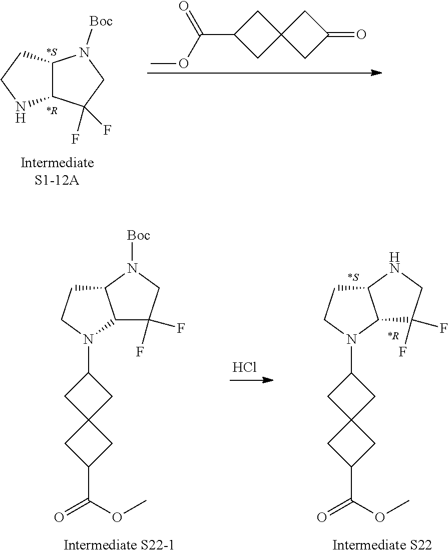

### S22-1: (cis)-tert-Butyl 3,3-difluoro-4-(6-(methoxycarbonyl)spiro[3.3]heptan-2-yl)hexahydropyrrolo[3,2-b]pyrrole-1(2H)-carboxylate

To a solution of S1-12A (450 mg, 90% purity, 1.63 mmol) in dichloromethane (10 mL) was added acetic acid (5 mL), methyl 6-oxospiro[3.3]heptane-2-carboxylate (410 mg, 2.44 mmol) and 1 M triisopropoxytitanium(IV) chloride in dichloromethane (3.1 mL, 3.1 mmol). After stirred at 25° C. for 30 minutes, sodium triacetoxyborohydride (1.7 g, 8.02 mmol) was added. After stirred at 25° C. for 16 hours, the reaction mixture was quenched with saturated sodium bicarbonate aqueous solution (30 mL) and filtered. The filtrate was extracted with dichloromethane (50 mL) for three times. The combined organic phases were washed with brine (50 mL), dried over Na₂SO₄₍ₛ₎ and filtered. The filtrate was concentrated to give a residue, which was purified by C-18 (acetonitrile:water=50% to 70%) to give the title compound (500 mg, 90% purity from ¹H NMR, 69% yield) as colorless oil. LC-MS (ESI): mass calcd. for C₂₀H₃₀F₂N₂O₄, 400.4, m/z found 401.5 [M+H]⁺. ¹H NMR (400 MHz, CDCl₃) δ 4.54-4.46 (m, 0.5H), 4.42-4.36 (m, 0.5H), 3.86-3.78 (m, 1H), 3.66 (s, 3H), 3.62-3.52 (m, 1H), 3.28-3.14 (m, 2H), 3.06-2.97 (m, 2H), 2.60-2.47 (m, 1H), 2.37-1.92 (m, 10H), 1.45 (s, 9H).

### S22: Methyl 6-((cis)-6,6-difluorohexahydropyrrolo[3,2-b]pyrrol-1(2H)-yl)spiro[3.3]heptane-2-carboxylate hydrochloride

The solution of S22-1 (100 mg, 90% purity, 0.225 mmol) in 4 M hydrochloride in ethyl acetate (3.5 mL, 17.5 mmol) was stirred at room temperature under nitrogen atmosphere for 1 hour. Then the reaction mixture was concentrated under reduced pressure to give the title compound (80 mg, 62% purity, 66% yield) as white solids. LC-MS (ESI): mass calcd. for C₁₅H₂₃ClF₂N₂O₂ 300.4, m/z found 301.4 [M+H]⁺.

### Compound 113-M: ethyl (S)-6-(((cis)-3,3-difluoro-4-(6-(methoxycarbonyl)spiro[3.3]heptan-2-yl)hexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

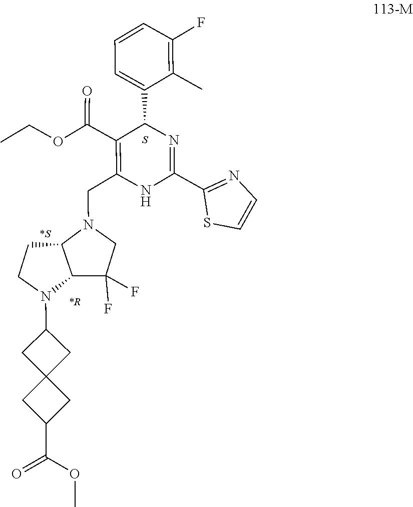

This compound was made from H2-1A with S22 according to Typical coupling method 1. Purified by C18 column (acetonitrile:water=60% to 70%) to give the title compound (400 mg, 90% purity from ¹H NMR, 69% yield) as light yellow solids. LC-MS (ESI): mass calcd. for C₃₃H₃₈F₃N₅O₄S, 657.7, m/z found 658.5 [M+H]⁺. ¹H NMR (400 MHz, CDCl₃) δ 9.31 (s, 1H), 7.82 (d, J=3.2 Hz, 1H), 7.40 (d, J=3.2 Hz, 1H), 7.09-7.03 (m, 1H), 7.02-6.97 (m, 1H), 6.92-6.88 (m, 1H), 6.00 (s, 1H), 4.25 (d, J=17.6 Hz, 1H), 4.10 (d, J=18.4 Hz, 1H), 4.03-4.01 (m, 2H), 3.83-3.77 (m, 1H), 3.67 (s, 3H), 3.38-3.21 (m, 3H), 3.12-2.92 (m, 3H), 2.56-2.50 (m, 4H), 2.35-2.21 (m, 4H), 2.15-1.87 (m, 6H), 1.07 (d, J=7.6 Hz, 3H).

Compound 113-M (150 mg, 90% purity, 0.205 mmol) was separated by chiral Prep. HPLC (Column: Chiralpak IG 5 μm 20*250 mm; Mobile Phase: Hex:EtOH=85:15 at 18 mL/min; Temp: 35° C.; Wavelength: 214 nm) to give the title compounds 113-A (60 mg, 40% yield, 90% purity from ¹H NMR, 100% stereopure) and 113-B (51 mg, 34% yield, 90% purity from ¹H NMR, 99.3% stereopure) as yellow solids.

113-A: LC-MS (ESI): mass calcd. for C₃₃H₃₈F₃N₅O₄S 657.7, m/z found 658.4 [M+H]f. Chiral analysis (Column: Chiralpak IG 5 μm 4.6*250 mm; Mobile Phase: Hex:EtOH=85:15 at 1 mL/min; Wavelength: 254 nm, R_{T}=12.624 min). ¹H NMR (400 MHz, CDCl₃) δ 9.30 (s, 1H), 7.82 (d, J=3.2 Hz, 1H), 7.39 (d, J=3.2 Hz, 1H), 7.07-7.04 (m, 1H), 6.99 (d, J=7.2 Hz, 1H), 6.92-6.88 (m, 1H), 6.00 (s, 1H), 4.25 (d, J=17.6 Hz, 1H), 4.10 (d, J=17.6 Hz, 1H), 4.03-4.00 (m, 2H), 3.82-3.76 (m, 1H), 3.67 (s, 3H), 3.35-3.19 (m, 3H), 3.11-2.92 (m, 3H), 2.54-2.53 (m, 4H), 2.35-1.87 (m, 10H), 1.07 (d, J=7.2 Hz, 3H).

113-B: LC-MS (ESI): mass calcd. for C₃₃H₃₈F₃N₅O₄S 657.7, m/z found 658.4 [M+H]⁺. Chiral analysis (Column: Chiralpak IG 5 μm 4.6*250 mm; Mobile Phase: Hex:EtOH=85:15 at 1 mL/min; Wavelength: 254 nm, R_{T}=14.007 min). ¹H NMR (400 MHz, CDCl₃) δ 9.30 (s, 1H), 7.82 (d, J=3.2 Hz, 1H), 7.39 (d, J=3.2 Hz, 1H), 7.07-7.04 (m, 1H), 6.99 (d, J=7.2 Hz, 1H), 6.92-6.88 (m, 1H), 6.00 (s, 1H), 4.25 (d, J=17.6 Hz, 1H), 4.10 (d, J=17.6 Hz, 1H), 4.03-4.00 (m, 2H), 3.82-3.76 (m, 1H), 3.67 (s, 3H), 3.35-3.19 (m, 3H), 3.11-2.92 (m, 3H), 2.54-2.53 (m, 4H), 2.35-1.87 (m, 10H), 1.07 (d, J=7.2 Hz, 3H).

### Compound 113: 6-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)spiro[3.3]heptane-2-carboxylic acid

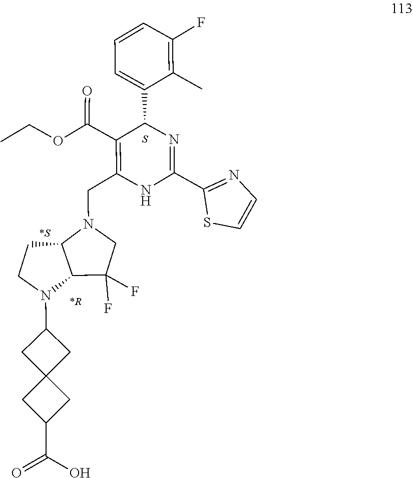

This compound was made from compound 113-M according to typical method 4. Purified by Prep. HPLC (Column: Waters Xbridge C18 (5 μm 19*150 mm), Mobile Phase A: water (+0.1% ammonium bicarbonate), Mobile Phase B: acetonitrile, UV: 254 nm, Flow rate: 15 mL/min, Gradient: 25-60% (% B)) to give the title compound (26.2 mg, 98% purity, 34% yield) as yellow solids. LC-MS (ESI): mass calcd. for C₃₂H₃₆F₃N₅O₄S, 643.7, m/z found 644.3 [M+H]⁺. ¹H NMR (400 MHz, CD₃OD) δ 7.78 (d, J=2.8 Hz, 1H), 7.60 (d, J=3.2 Hz, 1H), 7.04-6.97 (m, 2H), 6.83 (t, J=9.2 Hz, 1H), 5.86 (s, 1H), 4.16-4.12 (m, 1H), 4.01-3.92 (m, 3H), 3.76-3.74 (m, 1H), 3.34-3.26 (m, 1H), 3.15-3.09 (m, 2H), 3.01-2.90 (m, 3H), 2.55-2.51 (m, 1H), 2.40 (d, J=1.6 Hz, 3H), 2.20-1.77 (m, 10H), 1.07 (d, J=7.2 Hz, 3H).

### Compound 113A: 6-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)spiro[3.3]heptane-2-carboxylic acid

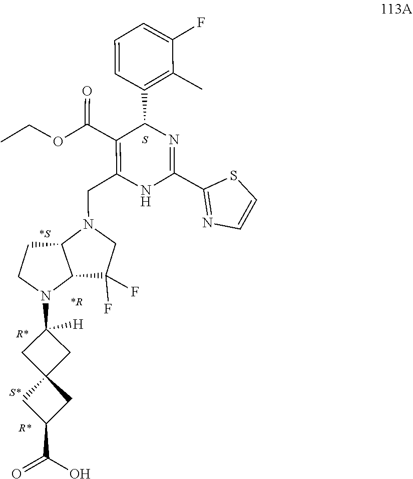

This compound was made from 113-A under similar condition. Purified by Prep. HPLC (Column: Waters Xbridge C18 (5 μm 19*150 mm), Mobile Phase A: water (+0.1% ammonium bicarbonate), Mobile Phase B: acetonitrile, UV: 254 nm, Flow rate: 15 mL/min, Gradient: 10-40% (% B)) to give the title compound (23.2 mg, 96.6% purity, 42% yield) as yellow solids. LC-MS (ESI): mass calcd. for C₃₂H₃₆F₃N₅O₄S 643.7, m/z found 644.3 [M+H]⁺. ¹H NMR (400 MHz, CD₃OD) δ 7.91 (d, J=3.2 Hz, 1H), 7.72 (d, J=3.2 Hz, 1H), 7.18-7.09 (m, 2H), 6.97-6.92 (m, 1H), 5.98 (s, 1H), 4.26 (d, J=16.8 Hz, 1H), 4.13-4.04 (m, 3H), 3.89-3.84 (m, 1H), 3.45-3.37 (m, 1H), 3.27-3.19 (m, 2H), 3.13-3.00 (m, 3H), 2.65-2.59 (m, 1H), 2.52 (d, J=2.0 Hz, 3H), 2.32-1.87 (m, 10H), 1.14 (d, J=6.8 Hz, 3H).

### Compound 113B: 6-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)spiro[3.3]heptane-2-carboxylic acid

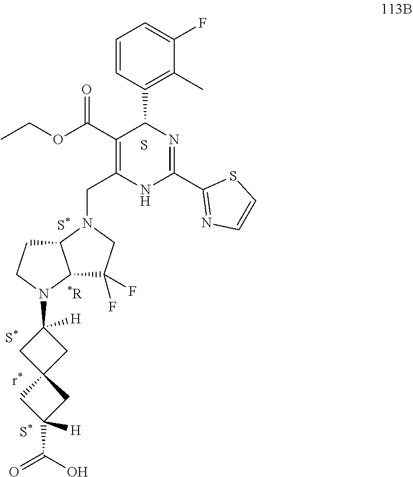

This compound was made from compound 113-B under similar condition. Purified by Prep. HPLC (Column: Waters Xbridge C18 (5 μm 19*150 mm), Mobile Phase A: water (+0.1% ammonium bicarbonate), Mobile Phase B: acetonitrile, UV: 254 nm, Flow rate: 15 mL/min, Gradient: 20-65% (% B)) to give the title compound (16 mg, 96.2% purity, 35% yield) as yellow solids. LC-MS (ESI): mass calcd. for C₃₂H₃₆F₃N₅O₄S 643.7, m/z found 644.3 [M+H]⁺. ¹H NMR (400 MHz, CD₃OD) δ 7.91 (d, J=3.2 Hz, 1H), 7.72 (d, J=3.2 Hz, 1H), 7.16-7.09 (m, 2H), 6.97-6.92 (m, 1H), 5.98 (s, 1H), 4.26 (d, J=16.8 Hz, 1H), 4.13-4.04 (m, 3H), 3.88-3.84 (m, 1H), 3.45-3.39 (m, 1H), 3.27-3.20 (m, 2H), 3.12-2.99 (m, 3H), 2.65-2.58 (m, 1H), 2.52 (d, J=2.0 Hz, 3H), 2.32-1.87 (m, 10H), 1.13 (d, J=7.2 Hz, 3H).

### Compound 115: 3-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)cyclopentane-1-carboxylic acid

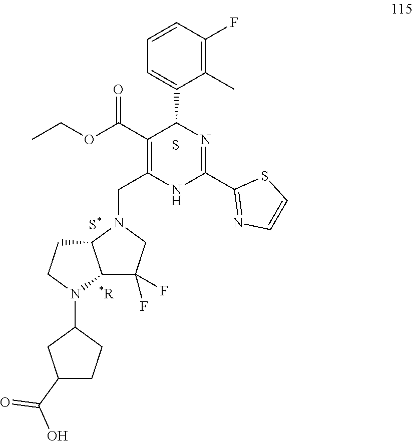

This compound was made from Compound 103 and Allyl 3-oxocyclopentanecarboxylate using typical method 5 and 2. Purified by Pre. HPLC (Column: Xbridge C18 (5 μm 19*150 mm), Mobile Phase A: water (0.1% ammonium bicarbonate), Mobile Phase B: acetonitrile, MS, Flow rate: 15 mL/min, Gradient: 25-65% (% B)) to give the title compound (25 mg, 42% yield, 99.2% purity) as yellow solids. LC-MS (ESI): mass calcd. for C₃₀H₃₄F₃N₅O₄S 617.2, m/z found 618.2 [M+H]⁺. ¹H NMR (400 MHz, CD₃OD) δ 7.93 (s, 1H), 7.74 (s, 1H), 7.25-7.09 (m, 2H), 6.96 (t, J=8.8 Hz, 1H), 5.99 (s, 1H), 4.31-4.26 (m, 1H), 4.18-4.05 (m, 3H), 3.97-3.86 (m, 1H), 3.68-3.50 (m, 1H), 3.29-2.91 (m, 4H), 2.89-2.69 (m, 2H), 2.53 (s, 3H), 2.26-2.08 (m, 1H), 2.07-1.58 (m, 7H), 1.17-1.13 (m, 3H).

### Compound 116A and 116B: 3-(((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)methyl)-1-methylcyclobutane-1-carboxylic acid

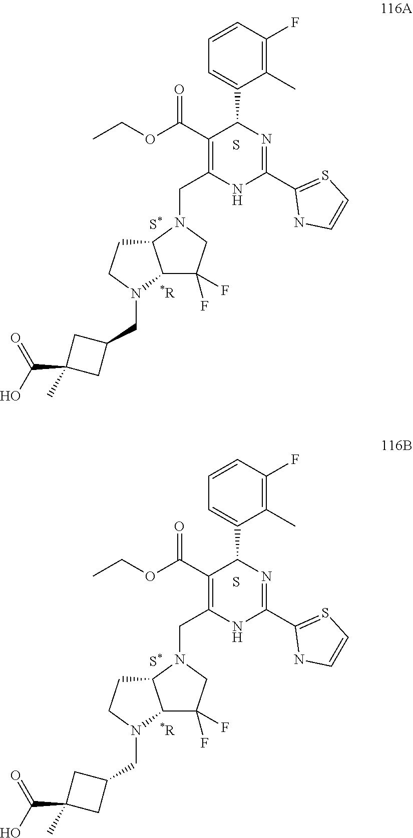

### Preparation of Intermediate S25

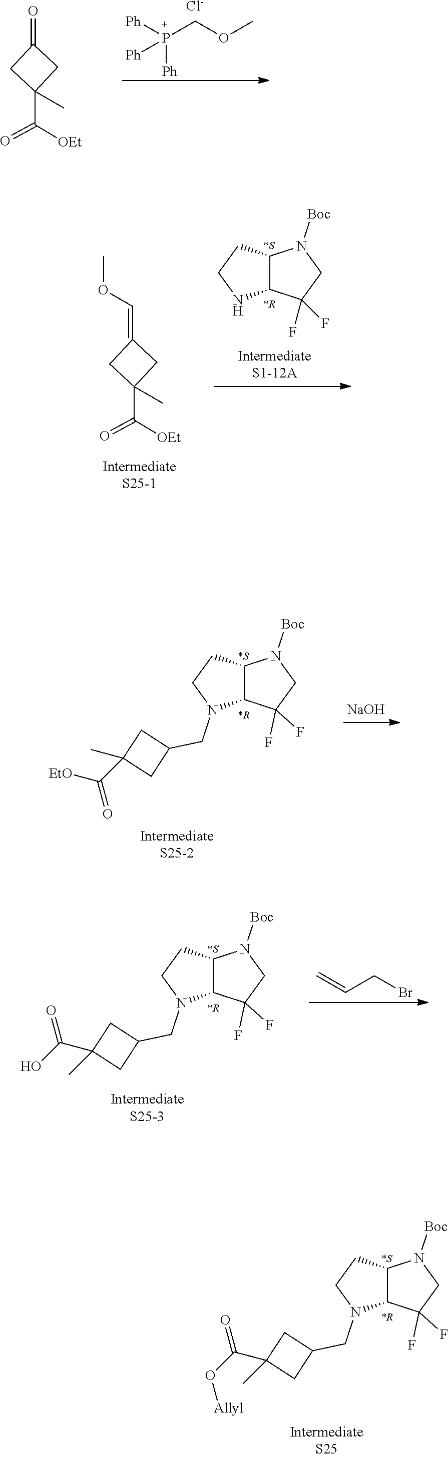

### S25-1: ethyl 3-(methoxymethylene)-1-methylcyclobutane-1-carboxylate

To a solution of (methoxymethyl)triphenylphosphonium chloride (80.0 g, 233 mmol) in tetrahydrofuran (400 mL) was added potassium tert-butoxide (26.0 g, 232 mmol) at 0° C. The brown mixture was stirred for 2 hours at 0° C. A solution of ethyl 1-methyl-3-oxocyclobutanecarboxylate (25.0 g, 160 mmol) in tetrahydrofuran (50 mL) was added at 0° C. and the mixture was stirred at room temperature overnight. Water (1000 mL) was added and the mixture was extracted with tert-butyl methyl ether (200 mL) twice. The combined organic layers were concentrated and purified by silica gel column chromatography (petroleum ether:ethyl acetate=20:1) to give the title compound (11.0 g, 60% purity from ¹H NMR, 22% yield) as yellow oil. ¹H NMR (400 MHz, CDCl₃) δ 5.89-5.82 (m, 1H), 4.15 (q, J=7.2 Hz, 2H), 3.36 (s, 3H), 3.16-3.07 (m, 2H), 2.54-2.37 (m, 2H), 1.30-1.24 (m, 6H).

### S25-2: (cis)-tert-Butyl 4-((3-(ethoxycarbonyl)-3-methylcyclobutyl)methyl)-3,3-difluorohexahydropyrrolo[3,2-b]pyrrole-1(2H)-carboxylate

To a mixture of S25-1 (2.00 g, 60% purity, 6.51 mmol) in tetrahydrofuran (15 mL) was added 6 M hydrochloride aqueous solution (4.0 mL) at 0° C. After stirred at 0° C. for 2 hours, brine (50 mL) was added. The organic layer was separated, dried over Na₂SO₄₍ₛ₎ and filtered. To the filtrate was added S1-12A (400 mg, 90% purity, 1.45 mmol), dichloromethane (15 mL), acetic acid glacial (900 mg, 15.0 mmol) and 1 M chlorotriisopropoxytitanium(IV) in dichloromethane (2.9 mL, 2.9 mmol). After stirred at room temperature for 1 hour, sodium triacetoxyborohydride (1.50 g, 7.08 mmol) was added by pointwise. The reaction mixture was stirred at room temperature overnight. Then saturated sodium bicarbonate aqueous solution (100 mL) was added and the mixture was extracted with dichloromethane (50 mL) twice. The combined organic phases were washed with brine (150 mL), dried over Na₂SO₄₍ₛ₎, filtered and concentrated. The residue was purified by C18 column (acetonitrile:water (0.1% ammonium bicarbonate)=20% to 80%) to give the title compound (400 mg, 96% purity, 66% yield) as yellow oil. LC-MS (ESI): mass calcd. for C₂₀H₃₂F₂N₂O₄ 402.2, m/z found 403.2 [M+H]⁺. ¹H NMR (400 MHz, CDCl₃) δ 4.50-4.31 (m, 1H), 4.18-4.10 (m, 2H), 3.91-3.75 (m, 1H), 3.70-3.55 (m, 1H), 3.19-2.91 (m, 3H), 2.64-2.20 (m, 5H), 2.16-2.09 (m, 1H), 1.98-1.90 (m, 1H), 1.88-1.75 (m, 1H), 1.69-1.65 (m, 1H), 1.45 (s, 9H), 1.41 (s, 1.5H), 1.33 (s, 1.5H), 1.29-1.24 (m, 3H).

### S25-3: 3-(((cis)-4-(tert-Butoxycarbonyl)-6,6-difluorohexahydropyrrolo[3,2-b]pyrrol-1(2H)-yl)methyl)-1-methylcyclobutanecarboxylic acid

To a solution of S25-2 (500 mg, 96% purity, 1.19 mmol) in methanol (4 mL) and tetrahydrofuran (4 mL) was added sodium hydroxide (140 mg, 3.50 mmol) in 2 mL of water. Then, the reaction mixture was stirred at room temperature overnight. The mixture was concentrated to give a residue, which was diluted with water (20 mL), extracted with ethyl acetate (30 mL) twice. The aqueous phase was acidified by 1 M hydrochloride solution to pH 4 and extracted with ethyl acetate (30 mL) twice. The combined extracts were dried over Na₂SO₄₍ₛ₎, filtered and concentrated to give the title compound (430 mg, 90% purity from ¹H NMR, 87% yield) as yellow solids. ¹H NMR (400 MHz, CDCl₃) δ 4.51-4.31 (m, 1H), 3.90-3.74 (m, 1H), 3.71-3.55 (m, 1H), 3.19-3.10 (m, 1H), 3.09-2.89 (m, 2H), 2.68-2.49 (m, 2H), 2.44-2.17 (m, 4H), 2.01-1.96 (m, 1H), 1.88-1.77 (m, 1H), 1.74-1.59 (m, 1H), 1.45 (m, 10.5H), 1.37 (s, 1.5H).

### S25: (cis)-tert-Butyl 4-((3-((allyloxy)carbonyl)-3-methylcyclobutyl)methyl)-3,3-difluorohexahydropyrrolo[3,2-b]pyrrole-1(2H)-carboxylate

To a mixture of S25-3 (430 mg, 90% purity, 1.03 mmol) and potassium carbonate (280 mg, 2.03 mmol) in N,N-dimethylformamide (3 mL) was added allyl bromide (190 mg, 1.57 mmol) at 0° C. The mixture was stirred at room temperature for 2 hours. Then it was filtered and the filtrate was purified by C18 column (acetonitrile:water (0.1% ammonium bicarbonate)=5% to 80%) to give the crude compound (420 mg, 90% purity from ¹H NMR, 88% yield) as yellow oil. ¹H NMR (400 MHz, CDCl₃) δ 5.98-5.87 (m, 1H), 5.34-5.21 (m, 2H), 4.62-4.56 (m, 2H), 4.49-4.30 (m, 1H), 3.90-3.74 (m, 1H), 3.72-3.55 (m, 1H), 3.18-3.08 (m, 1H), 3.07-2.88 (m, 2H), 2.65-2.37 (m, 3H), 2.32-2.13 (m, 3H), 2.01-1.95 (m, 1H), 1.87-1.76 (m, 1H), 1.72-1.67 (m, 0.5H), 1.61-1.56 (m, 0.5H), 1.45 (s, 9H), 1.44 (s, 1.5H), 1.36 (s, 1.5H).

### Compound 116-M: ethyl (S)-6-(((cis)-4-((3-((allyloxy)carbonyl)-3-methylcyclobutyl)methyl)-3,3-difluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

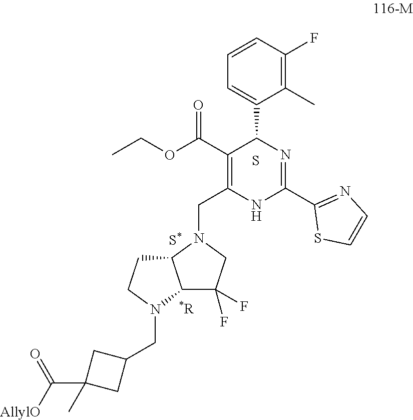

This compound was made from H2-1A with S25 according to Typical coupling method 1. Purified by C18 column (acetonitrile:water (0.1% ammonium bicarbonate)=5% to 70%) to give the title compound (560 mg, 90% purity from ¹H NMR, 82% yield) as yellow oil. LC-MS (ESI): mass calcd. for C₃₄H₄₀F₃N₅O₄S 671.3, m/z found 672.5 [M+H]⁺. ¹H NMR (400 MHz, CDCl₃) δ 9.36 (s, 1H), 7.82 (d, J=3.2 Hz, 1H), 7.40 (d, J=3.2 Hz, 1H), 7.10-7.04 (m, 1H), 6.99-6.97 (m, 1H), 6.92-6.88 (m, 1H), 6.00 (s, 1H), 5.98-5.88 (m, 1H), 5.35-5.29 (m, 1H), 5.24-5.22 (m, 1H), 4.63-4.58 (m, 2H), 4.22-4.10 (m, 2H), 4.08-3.99 (m, 2H), 3.88-3.77 (m, 1H), 3.38-3.25 (m, 1H), 3.22-3.10 (m, 2H), 3.04-2.79 (m, 2H), 2.70-2.48 (m, 5H), 2.42-2.30 (m, 1H), 2.24-2.17 (m, 1H), 2.06-2.00 (m, 1H), 1.97-1.84 (m, 2H), 1.75-1.62 (m, 2H), 1.46 (s, 1.6H), 1.38 (s, 1.4H), 1.11 (t, J=6.8 Hz, 3H).

116-M was chiral separated (Column: Superchiral S-AD 5 μm 21*250 mm; Mobile Phase: hexane:IPA=90:10 at 15 mL/min; Temp: 35° C.; Wavelength: 254 nm) and converted to 116A and 116B according to typical method 2.

116A: purified by C18 column (acetonitrile:water (0.1% ammonium bicarbonate)=5% to 80%) to give the title compound (60 mg, 98.7% purity, 46% yield) as yellow solids. LC-MS (ESI): mass calcd. for C₃₁H₃₆F₃N₅O₄S 631.2, m/z found 632.3 [M+H]⁺. ¹H NMR (400 MHz, CDCl₃) δ 9.35 (br s, 1H), 7.82 (d, J=3.2 Hz, 1H), 7.40 (d, J=2.8 Hz, 1H), 7.10-7.04 (m, 1H), 6.98 (d, J=7.6 Hz, 1H), 6.90 (t, J=8.8 Hz, 1H), 6.00 (s, 1H), 4.20 (d, J=17.2 Hz, 1H), 4.11 (d, J=17.2 Hz, 1H), 4.08-3.99 (m, 2H), 3.82 (q, J=7.2 Hz, 1H), 3.35-3.13 (m, 3H), 3.01-2.82 (m, 2H), 2.67-2.56 (m, 2H), 2.54 (d, J=1.2 Hz, 3H), 2.46-2.38 (m, 1H), 2.26-2.19 (m, 2H), 2.06-1.94 (m, 4H), 1.47 (s, 3H), 1.11 (t, J=7.2 Hz, 3H).

116B: purified by C18 column (acetonitrile:water (0.1% ammonium bicarbonate)=5% to 80%) to give the title compound (26 mg, 96.3% purity, 23% yield) as yellow solids. LC-MS (ESI): mass calcd. for C₃₁H₃₆F₃N₅O₄S 631.2, m/z found 632.3 [M+H]⁺. ¹H NMR (400 MHz, CDCl₃) δ 9.36 (br s, 1H), 7.82 (d, J=3.2 Hz, 1H), 7.40 (d, J=3.2 Hz, 1H), 7.10-7.04 (m, 1H), 6.98 (d, J=7.6 Hz, 1H), 6.90 (t, J=8.8 Hz, 1H), 6.00 (s, 1H), 4.20 (d, J=16.8 Hz, 1H), 4.12 (d, J=16.8 Hz, 1H), 4.08-3.99 (m, 2H), 3.82 (q, J=7.2 Hz, 1H), 3.37-3.27 (m, 1H), 3.24-3.13 (m, 2H), 3.00-2.86 (m, 2H), 2.71-2.58 (m, 4H), 2.54 (s, 3H), 2.42-2.33 (m, 1H), 1.99-1.91 (m, 2H), 1.77-1.73 (m, 1H), 1.68-1.63 (m, 1H), 1.39 (s, 3H), 1.11 (t, J=7.2 Hz, 3H).

### Compound 117: 1-(2-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)ethyl)cyclopropane-1-carboxylic acid (Single Diastereomer)

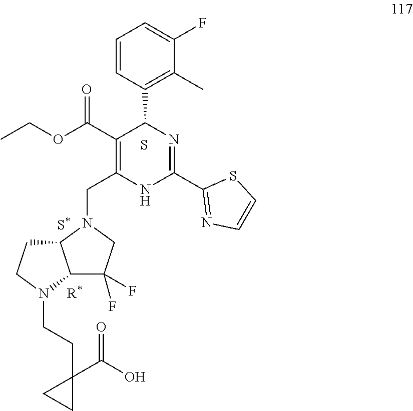

This compound was made from Compound 103 and tert-butyl 1-(2-oxoethyl)cyclopropane-1-carboxylate using typical method 5 and 3 successively. Purified by C18 (acetonitrile:water (0.1% ammonium bicarbonate)=10% to 90%) to get the desired compound (20 mg, 97.3% purity, 47% yield) as yellow solids. LC-MS (ESI): mass calcd. for C₃₀H₃₄F₃N₅O₄S 617.7, m/z found 618.3 [M+H]⁺. ¹H NMR (400 MHz, CDCl₃) δ 9.21 (br s, 1H), 7.85 (d, J=3.2 Hz, 1H), 7.40 (d, J=3.2 Hz, 1H), 7.11-7.05 (m, 1H), 6.98 (d, J=7.2 Hz, 1H), 6.91 (t, J=8.8 Hz, 1H), 6.00 (s, 1H), 4.28-4.24 (m, 1H), 4.16-4.11 (m, 1H), 4.08-4.00 (m, 2H), 3.93-3.88 (m, 1H), 3.57-3.38 (m, 3H), 3.23-3.17 (m, 1H), 3.07-2.97 (m, 1H), 2.92-2.85 (m, 1H), 2.72-2.62 (m, 1H), 2.54-2.53 (m, 3H), 2.16-2.00 (m, 3H), 1.50-1.43 (m, 3H), 1.30-1.24 (m, 1H), 1.11 (t, J=7.2 Hz, 3H), 0.75-0.64 (m, 2H).

### Compound 118: 4-((cis)-4-(((S)-5-(ethoxycarbonyl)-6-(3-fluoro-2-methylphenyl)-2-(4-methylthiazol-2-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

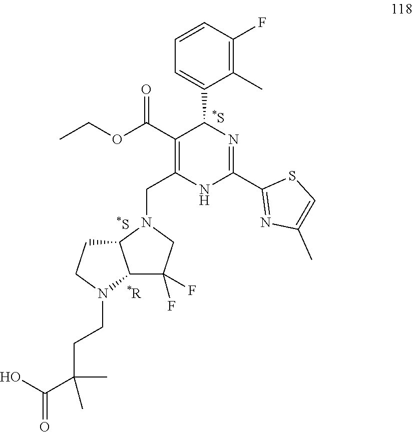

This compound was made from H22-1B and S9 analogous to compound 7A. Purified by C18 column (acetonitrile:water (+0.02% ammonium bicarbonate)=05% to 70%) to give the title compound (80 mg, 99.3% purity, 90% yield) as yellow solids. LC-MS (ESI): mass calcd. for C₃₁H₃₈F₃N₅O₄S 633.3, m/z found 634.3 [M+H]⁺. ¹H NMR (400 MHz, CD₃OD) δ 7.29 (s, 1H), 7.19-7.10 (m, 2H), 6.98-6.93 (m, 1H), 5.97 (s, 1H), 4.26 (d, J=16.8 Hz, 1H), 4.16-4.05 (m, 3H), 3.94-3.89 (m, 1H), 3.46-3.37 (m, 2H), 3.31-3.29 (m, 1H), 3.11-3.03 (m, 1H), 2.95-2.88 (m, 1H), 2.71-2.57 (m, 2H), 2.52 (d, J=2.0 Hz, 3H), 2.47 (s, 3H), 2.06-1.93 (m, 2H), 1.87-1.83 (m, 2H), 1.24 (s, 6H), 1.15 (t, J=7.2 Hz, 3H).

### Preparation of Intermediate S31

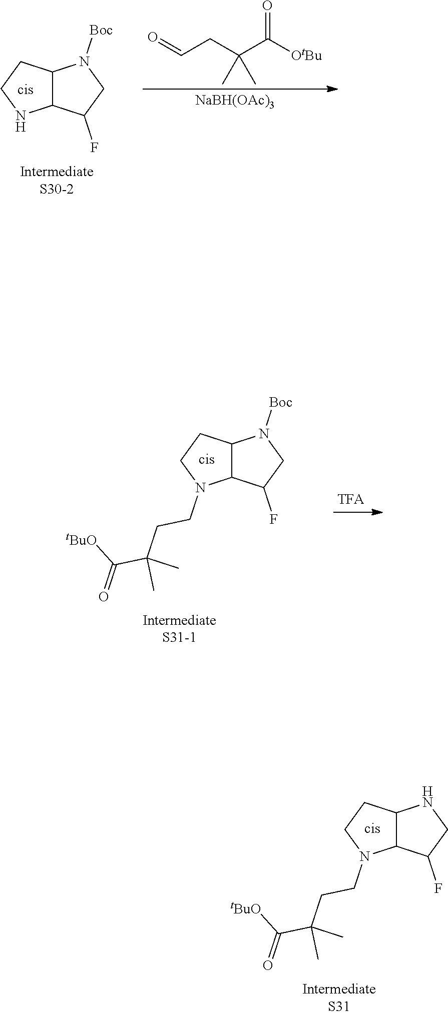 [SMILES: CC(C)(C)OC(=O)N1CC(F)C2NCCC21 ; CC(C)(C)OC(=O)C(C)(C)CC=O ; "TFA" ; CC(C)(C)OC(=O)N1CC(F)C2C1CCN2CCC(C)(C)C(=O)OC(C)(C)C ; "NaBH(OAc)3" ; CC(C)(C)OC(=O)C(C)(C)CCN1CCC2NCC(F)C21]

S31-1:-4-(4-(tert-butoxy)-3,3-dimethyl-4-oxobutyl)-3-fluorohexahydropyrrolo[3,2-b]pyrrole-1(2H)-carboxylate, prepared from S30-2 and intermediate S9-1 analogous to S30-3. ¹H NMR (400 MHz, CHLOROFORM-d) δ 4.87-4.73 (dt, 1H), 4.47-4.32 (m, 1H), 3.72-3.65 (m, 2H), 3.63-3.47 (m, 1H), 3.17-3.09 (m, 1H), 3.07-2.98 (m, 1H), 2.80-2.69 (m, 1H), 2.35-2.19 (m, 2H), 2.18-2.06 (m, 1H), 1.74-1.65 (m, 2H), 1.48-1.42 (m, 18H), 1.15-1.12 (m, 6H).

### S31: tert-butyl 4-(-6-fluorohexahydropyrrolo[3,2-b]pyrrol-1(2H)-yl)-2,2-dimethylbutanoate

To a solution of S31-1 (120 mg, 0.3 mmol) in DCM (9 mL) was added TFA (1 mL). The mixture was stirred at room temperature for 16 hours. Then the mixture was poured into aq.NaHCO₃ (30 mL) and extracted with DCM (25 mL×3). The combined organic phase was dried over Na₂SO₄ and filtered. The filtrate was concentrated in vacuo to give the title compound (32 mg, yield 35%), which was used directly in the next step without further purification. LC-MS (ESI): mass calcd. for C16H29FN2O2 300.2, m/z found 301.3[M+H]⁺.

### Compound 119-M: (cis)-ethyl (4S)-6-((4-(4-(tert-butoxy)-3,3-dimethyl-4-oxobutyl)-3-fluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

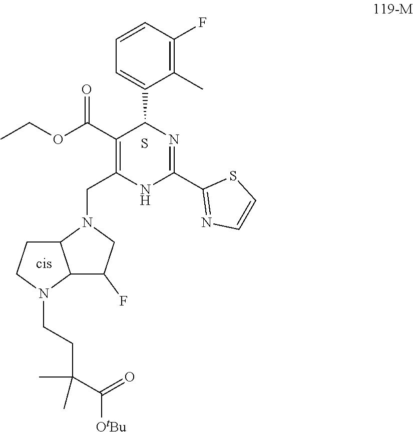

This compound was made from H2-1A with S31 according to Typical coupling method 1. LC-MS (ESI): mass calcd. for C₃₄H₄₅F₂N₅O₄S 657.3, m/z found 658.4 [M+H]⁺.

Compound 119-M (46 mg, 90% purity, 0.063 mmol) was separated by chiral Prep.HPLC (Column: Chiralpak ID 5 μm 30*250 mm; Mobile Phase: Hex:EtOH=95:5 at 30 mL/min; Temp: 30° C.; Wavelength: 254 nm) to afford the desired products 119-A (10 mg, 90% purity from ¹H NMR, 21% yield, 98.5% stereopure) and 119-B (15 mg, 90% purity from ¹H NMR, 32% yield, 95.5% stereopure) as yellow solids.

119-A: Chiral analysis (Column: Chiralpak ID 5 μm 4.6*250 mm; Mobile Phase: Hex:EtOH=95:5 at 1.0 mL/min; Temp: 30° C.; Wavelength: 254 nm, R_{T}=7.819 min). ¹H NMR (400 MHz, CDCl₃) δ 7.85-7.81 (m, 1H), 7.42-7.38 (m, 1H), 7.08-6.90 (m, 3H), 6.01 (s, 1H), 4.38-4.20 (m, 1H), 4.09-3.98 (m, 4H), 3.93-3.80 (m, 1H), 3.65-3.48 (m, 1H), 3.30-3.16 (m, 2H), 3.07-2.98 (m, 1H), 2.89-2.68 (m, 1H), 2.54-2.48 (m, 4H), 2.32-2.12 (m, 2H), 1.90-1.74 (m, 2H), 1.46 (s, 9H), 1.26-1.20 (m, 6H), 1.12 (t, J=7.2 Hz, 3H).

119-B: Chiral analysis (Column: Chiralpak ID 5 μm 4.6*250 mm; Mobile Phase: Hex:EtOH=95:5 at 1.0 mL/min; Temp: 30° C.; Wavelength: 254 nm, R_{T}=9.118 min). ¹H NMR (400 MHz, CDCl₃) δ 7.85-7.81 (m, 1H), 7.44-7.40 (m, 1H), 7.10-6.98 (m, 2H), 6.92-6.88 (m, 1H), 6.01 (s, 1H), 4.95-4.77 (m, 1H), 4.24-4.15 (m, 2H), 4.09-3.98 (m, 3H), 3.85-3.82 (m, 1H), 3.72-3.58 (m, 1H), 3.26-3.03 (m, 3H), 2.72-2.55 (m, 1H), 2.55-2.47 (m, 4H), 2.29-2.19 (m, 2H), 1.91-1.74 (m, 2H), 1.46 (s, 9H), 1.30-1.20 (m, 6H), 1.12 (t, J=7.2 Hz, 3H).

### Compound 119: 4-(4-(((S)-5-(ethoxycarbonyl)-6-(3-fluoro-2-methylphenyl)-2-(thiazol-2-yl)-3,6-dihydropyrimidin-4-yl)methyl)-6-fluorohexahydropyrrolo[3,2-b]pyrrol-1(2H)-yl)-2,2-dimethylbutanoic acid (Single Diastereomer)

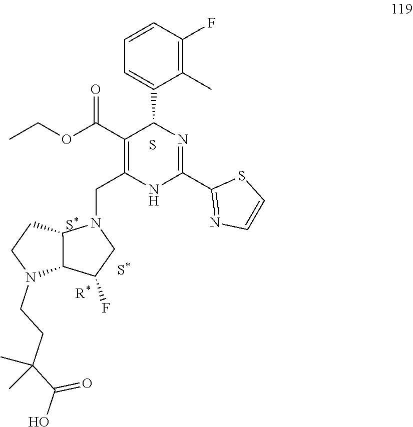

This compound was made from 119-B using typical method 3. Purified by C18 column (acetonitrile:water (0.5% ammonium bicarbonate)=5% to 70%) to afford the desired product (9 mg, 96.4% purity, 81% yield) as yellow solids. LC-MS (ESI): mass calcd. for C₃₀H₃₇F₂N₅O₄S 601.3, m/z found 602.3 [M+H]⁺. ¹H NMR (400 MHz, CD₃OD) δ 7.83 (d, J=3.2 Hz, 1H), 7.63 (d, J=3.2 Hz, 1H), 7.11-7.01 (m, 2H), 3.88-6.83 (m, 1H), 5.88 (s, 1H), 4.92 (d, J=51.2 Hz, 1H), 4.18 (d, J=16.8 Hz, 1H), 4.08 (d, J=17.2 Hz, 1H), 3.97 (q, J=7.2 Hz, 2H), 3.86-3.77 (m, 1H), 3.40-3.30 (m, 1H), 3.19-3.16 (m, 1H), 3.12-3.07 (m, 1H), 2.99-2.88 (m, 1H), 2.85-2.77 (m, 1H), 2.66-2.57 (m, 1H), 2.43-2.37 (m, 4H), 1.91-1.79 (m, 4H), 1.14 (s, 6H), 1.05 (t, J=7.2 Hz, 3H).

### Compound 120A and 120B: 6-((cis)-4-(((R)-6-(2,3-difluorophenyl)-5-(methoxycarbonyl)-2-(thiazol-2-yl)-3,6-dihydropyrimidin-4-yl)methyl)-6,6-difluorohexahydropyrrolo[3,2-b]pyrrol-1(2H)-yl)spiro[3.3]heptane-2-carboxylic acid

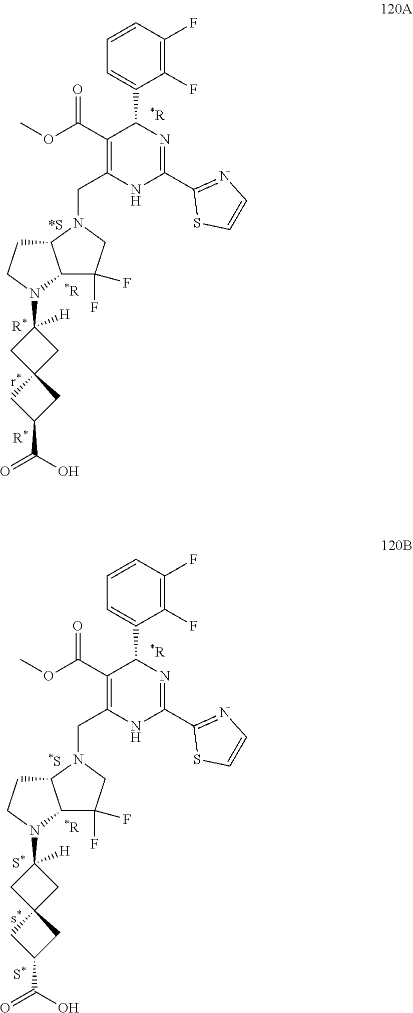

This compound was made from H123-1A and S22 using typical coupling method 1 and 4 analogous to compound compound 113A/B.

120A: LC-MS (ESI): mass calcd. for C₃₀H₃₁F₄N₅O₄S 633.2, m/z found 634.2 [M+H]⁺. ¹H NMR (400 MHz, CD₃OD) δ 7.94 (d, J=3.2 Hz, 1H), 7.76 (d, J=3.2 Hz, 1H), 7.21-7.08 (m, 3H), 6.05 (s, 1H), 4.22 (d, J=17.2 Hz, 1H), 4.07 (d, J=16.8 Hz, 1H), 3.88-3.83 (m, 1H), 3.64 (s, 3H), 3.45-3.37 (m, 1H), 3.29-3.19 (m, 2H), 3.13-3.00 (m, 3H), 2.65-2.59 (m, 1H), 2.32-2.08 (m, 6H), 2.06-1.85 (m, 4H).

120B: LC-MS (ESI): mass calcd. for C₃₀H₃₁F₄N₅O₄S 633.2, m/z found 634.2 [M+H]⁺. ¹H NMR (400 MHz, CD₃OD) δ 7.95 (d, J=3.2 Hz, 1H), 7.77 (d, J=3.2 Hz, 1H), 7.22-7.10 (m, 3H), 6.06 (s, 1H), 4.23 (d, J=16.8 Hz, 1H), 4.08 (d, J=17.2 Hz, 1H), 3.89-3.84 (m, 1H), 3.65 (s, 3H), 3.45-3.38 (m, 1H), 3.30-3.19 (m, 2H), 3.13-3.00 (m, 3H), 2.64-2.58 (m, 1H), 2.33-2.10 (m, 6H), 2.07-1.85 (m, 4H).

### Compound 121: 4-((cis)-4-(((R)-5-(ethoxycarbonyl)-6-(6-fluoro-2-methylpyridin-3-yl)-2-(thiazol-2-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

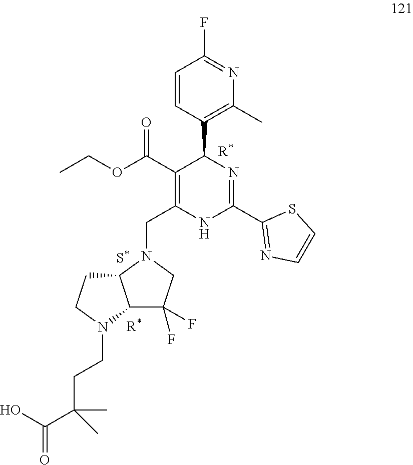

This compound was made from S9 and H20-1A analogous to compound 7A. Purified by C18 column (acetonitrile:water=35% to 70%) to give the title compound (55.4 mg, 99% purity, 55% yield) as yellow solids. LC-MS (ESI): mass calcd. for C₂₉H₃₅F₃N₆O₄S 620.7, m/z found 621.3 [M+H]⁺. ¹H NMR (400 MHz, CDCl₃) δ 9.30 (br s, 1H), 7.86 (d, J=3.2 Hz, 1H), 7.56-7.52 (m, 1H), 7.44 (d, J=2.4 Hz, 1H), 6.70-6.67 (m, 1H), 5.98 (s, 1H), 4.29 (d, J=17.6 Hz, 1H), 4.09-4.01 (m, 3H), 3.88-3.83 (m, 1H), 3.47-3.39 (m, 2H), 3.19-3.12 (m, 1H), 3.01-2.92 (m, 1H), 2.78-2.70 (m, 4H), 2.60-2.52 (m, 1H), 2.07-1.93 (m, 3H), 1.73-1.66 (m, 2H), 1.28 (s, 6H), 1.13 (t, J=7.2 Hz, 3H).

### Preparation of Intermediate S33

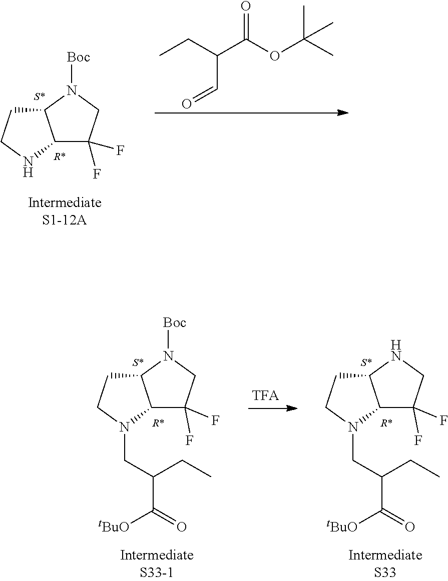 [SMILES: CC(C)(C)OC(=O)N1CC(F)(F)[C@@H]2NCC[C@@H]21 ; CCC(C=O)C(=O)OC(C)(C)C ; "TFA" ; CCC(CN1CC[C@H]2[C@@H]1C(F)(F)CN2C(=O)OC(C)(C)C)C(=O)OC(C)(C)C ; CCC(CN1CC[C@@H]2NCC(F)(F)[C@@H]21)C(=O)OC(C)(C)C]

### S33-1: (cis)-tert-Butyl 4-(2-(tert-butoxycarbonyl)butyl)-3,3-difluorohexahydropyrrolo[3,2-b]pyrrole-1(2H)-carboxylate

This intermediate was made from S1-12A and tert-butyl 2-formylbutanoate according to typical method 5. LC-MS (ESI): mass calcd. for C₂₀H₃₄F₂N₂O₄ 404.2, m/z found 405.3 [M+H]⁺. ¹H NMR (400 MHz, CDCl₃) δ 4.47-4.33 (m, 1H), 3.90-3.58 (m, 2H), 3.17-3.08 (m, 1.5H), 3.01-2.90 (m, 1H), 2.65-2.60 (m, 1H), 2.42-2.18 (m, 3.5H), 1.88-1.77 (m, 1H), 1.69-1.60 (m, 1H), 1.56-1.50 (m, 1H), 1.46-1.44 (m, 18H), 0.95-0.89 (m, 3H).

### S33: tert-Butyl 2-(((cis)-6,6-difluorohexahydropyrrolo[3,2-b]pyrrol-1(2H)-yl)methyl)butanoate

A solution of S33-1 (600 mg, 90% purity, 1.34 mmol) in dichloromethane (4 mL) and trifluoroacetic acid (1 mL) was stirred at 40° C. for 6 hours. The mixture was basified to pH 7-8 with saturated sodium bicarbonate aqueous solution, extracted with dichloromethane (20 mL) twice. The combined extracted was concentrated to give a residue, which was purified by C18 column (acetonitrile:water=5% to 95%) to give the title compound (300 mg, 90% purity from ¹H NMR, 66% yield) as brown oil. LC-MS (ESI): R_{T}=1.68 min, mass calcd. for C₁₅H₂₆F₂N₂O₂ 304.2, m/z found 305.2 [M+H]⁺. ¹H NMR (400 MHz, CDCl₃) δ 4.01-3.95 (m, 1H), 3.32-2.84 (m, 5H), 2.79-2.73 (m, 0.3H), 2.68-2.62 (m, 0.7H), 2.44-2.31 (m, 1H), 2.26-2.16 (m, 1H), 2.13-2.09 (m, 1H), 2.00 (br s, 1H), 1.72-1.52 (m, 3H), 1.45-1.44 (m, 9H), 0.91 (t, J=7.6 Hz, 3H).

### Compound 122-M: Ethyl 6-(((cis)-4-(2-(tert-butoxycarbonyl)butyl)-3,3-difluorohexahydropyrrolo[3,2-b]pyrrol-1(2H)-yl)methyl)-4-(6-fluoro-2-methylpyridin-3-yl)-2-(thiazol-2-yl)-1,4-dihydropyrimidine-5-carboxylate

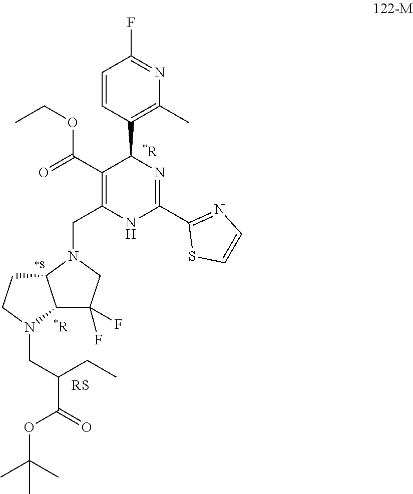

This compound was made from H20-1A and S33 according to typical coupling method 1. LC-MS (ESI): mass calcd. for C₃₂H₄₁F₃N₆O₄S 662.3, m/z found 663.5 [M+H]⁺. ¹H NMR (400 MHz, CDCl₃) δ 9.45 (s, 1H), 7.85-7.83 (m, 1H), 7.54 (t, J=8.4 Hz, 1H), 7.43 (d, J=3.2 Hz, 1H), 6.68 (dd, J=8.0, 3.2 Hz, 1H), 5.98 (s, 1H), 4.26-4.19 (m, 1H), 4.12-4.01 (m, 3H), 3.82-3.75 (m, 1H), 3.37-3.06 (m, 3H), 2.95-2.81 (m, 3H), 2.79 (s, 3H), 2.57-2.52 (m, 1H), 2.46-2.40 (m, 1H), 1.95-1.86 (m, 2H), 1.66-1.54 (m, 2H), 1.46 (s, 9H), 1.14 (t, J=7.2 Hz, 3H), 0.93 (t, J=7.2 Hz, 3H).

Chiral separation: (Column: Chiralpak IG 5 μm 20*250 mm; Mobile Phase: Hex:EtOH=80:20 at 15 mL/min; Temp: 30° C.; Wavelength: 254 nm) to give compounds 122-A (160 mg, 90% purity from ¹H NMR, 53% yield, 100% stereopure) and 122-B (65 mg, 90% purity from ¹H NMR, 22% yield, 99.9% stereopure) as yellow solids.

122-A: LC-MS (ESI): mass calcd. for C₃₂H₄₁F₃N₆O₄S 662.3, m/z found 663.3 [M+H]⁺. Chiral analysis (Column: Chiralpak IG 5 μm 4.6*250 mm; Mobile Phase: Hex:EtOH=80:20 at 1 mL/min; Temp: 30° C.; Wavelength: 254 nm, R_{T}=6.809 min). ¹H NMR (400 MHz, CDCl₃) δ 9.38 (br s, 1H), 7.83 (d, J=3.2 Hz, 1H), 7.54 (t, J=8.0 Hz, 1H), 7.43 (d, J=3.2 Hz, 1H), 6.68 (dd, J=8.4, 3.6 Hz, 1H), 5.98 (s, 1H), 4.24 (d, J=17.2 Hz, 1H), 4.10-4.00 (m, 3H), 3.81-3.76 (m, 1H), 3.39-3.24 (m, 2H), 3.17-3.14 (m, 1H), 3.04-2.82 (m, 3H), 2.79 (s, 3H), 2.58-2.53 (m, 1H), 2.48-2.40 (m, 1H), 1.94-1.87 (m, 2H), 1.67-1.58 (m, 2H), 1.46 (s, 9H), 1.14 (t, J=7.2 Hz, 3H), 0.93 (t, J=7.2 Hz, 3H).

122-B: LC-MS (ESI): mass calcd. for C₃₂H₄₁F₃N₆O₄S 662.3, m/z found 663.3 [M+H]⁺. Chiral analysis (Column: Chiralpak IG 5 μm 4.6*250 mm; Mobile Phase: Hex:EtOH=80:20 at 1 mL/min; Temp: 30° C.; Wavelength: 254 nm, R_{T}=9.057 min). ¹H NMR (400 MHz, CDCl₃) δ 9.33 (br s, 1H), 7.85 (d, J=3.2 Hz, 1H), 7.54 (t, J=8.4 Hz, 1H), 7.43 (d, J=3.2 Hz, 1H), 6.68 (dd, J=8.0, 3.2 Hz, 1H), 5.98 (s, 1H), 4.23 (d, J=17.6 Hz, 1H), 4.12-3.99 (m, 3H), 3.85-3.80 (m, 1H), 3.39-3.21 (m, 3H), 3.05-2.91 (m, 3H), 2.79 (s, 3H), 2.47-2.38 (m, 2H), 2.00-1.85 (m, 2H), 1.65-1.48 (m, 2H), 1.46 (s, 9H), 1.14 (t, J=7.2 Hz, 3H), 0.93 (t, J=7.2 Hz, 3H).

### Compound 122A: 2-(((cis)-4-((5-(ethoxycarbonyl)-6-(6-fluoro-2-methylpyridin-3-yl)-2-(thiazol-2-yl)-3,6-dihydropyrimidin-4-yl)methyl)-6,6-difluorohexahydropyrrolo[3,2-b]pyrrol-1(2H)-yl)methyl)butanoic acid (Single Diastereomer)

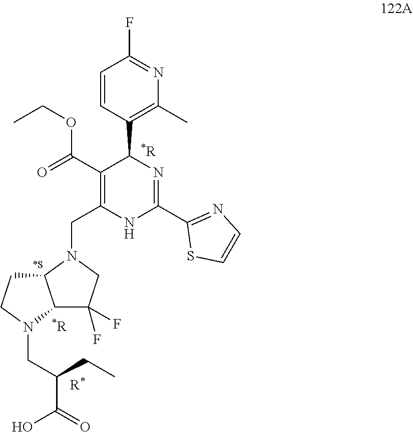

this compound was made from compound 122-A according to typical method 3. Purified by C18 column (acetonitrile:water=5% to 95%) to give the title compound (88.8 mg, 99.8% purity, 67% yield) as yellow solids. LC-MS (ESI): mass calcd. for C₂₈H₃₃F₃N₆O₄S 606.2, m/z found 607.3 [M+H]⁺. ¹H NMR (400 MHz, CDCl₃) δ 9.32 (s, 1H), 7.86 (d, J=3.2 Hz, 1H), 7.54 (t, J=8.0 Hz, 1H), 7.45 (d, J=3.2 Hz, 1H), 6.68 (dd, J=8.4, 3.6 Hz, 1H), 5.99 (s, 1H), 4.35 (d, J=17.6 Hz, 1H), 4.10-4.00 (m, 3H), 3.86-3.81 (m, 1H), 3.65-3.57 (m, 1H), 3.35-3.25 (m, 2H), 3.09-2.88 (m, 4H), 2.79 (s, 3H), 2.51-2.44 (m, 1H), 2.01-1.96 (m, 2H), 1.86-1.79 (m, 1H), 1.66-1.59 (m, 1H), 1.13 (t, J=7.2 Hz, 3H), 1.01 (t, J=7.2 Hz, 3H).

### Compound 123A and 123B: 2-(-3-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)cyclobutyl)acetic acid

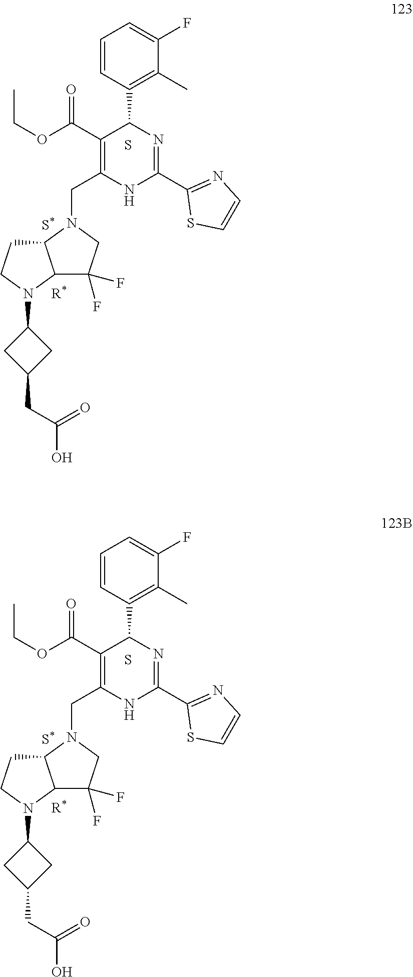

These compounds were made from Compound 103 and methyl 2-(3-oxocyclobutyl)acetate using typical method 5 and typical method 4 successively.

119A: yellow solid, LCMS (ESI): mass calcd. for C₃₀H₃₄F₃N₅O₄S 617.2, m/z found 618.3 [M+H]⁺. ¹H NMR (400 MHz, CDCl₃) δ 7.91 (d, J=4.0 Hz, 1H), 7.61 (d, J=4.0 Hz, 1H), 7.16-7.08 (m, 1H), 7.03 (m, 1H), 6.99-6.93 (m, 1H), 6.08 (s, 1H), 4.54-4.38 (m, 2H), 4.10-3.92 (m, 5H), 3.57-3.40 (m, 3H), 3.11-3.02 (m, 1H), 2.57-2.54 (m, 5H), 2.50-2.49 (m, 3H), 2.33-2.25 (m, 4H), 1.12 (s, 3H).

119B: yellow solid. LC-MS (ESI): mass calcd. for C₃₀H₃₄F₃N₅O₄S 617.2, m/z found 618.3 [M+H]⁺. ¹H NMR (400 MHz, CDCl₃) δ 7.90 (d, J=4.0 Hz, 1H), 7.58 (d, J=4.0 Hz, 1H), 7.15-7.07 (m, 1H), 7.04-7.00 (m, 1H), 6.98-6.89 (m, 1H), 6.07 (s, 1H), 4.59-4.39 (m, 2H), 4.27-4.16 (m, 1H), 4.11-4.01 (m, 4H), 3.50-3.37 (m, 3H), 3.13-2.98 (m, 1H), 2.85-2.70 (m, 3H), 2.59-2.53 (m, 2H), 2.51-2.49 (m, 3H), 2.22-2.16 (m, 4H), 1.11 (s, 3H).

### Preparation of Intermediate S45

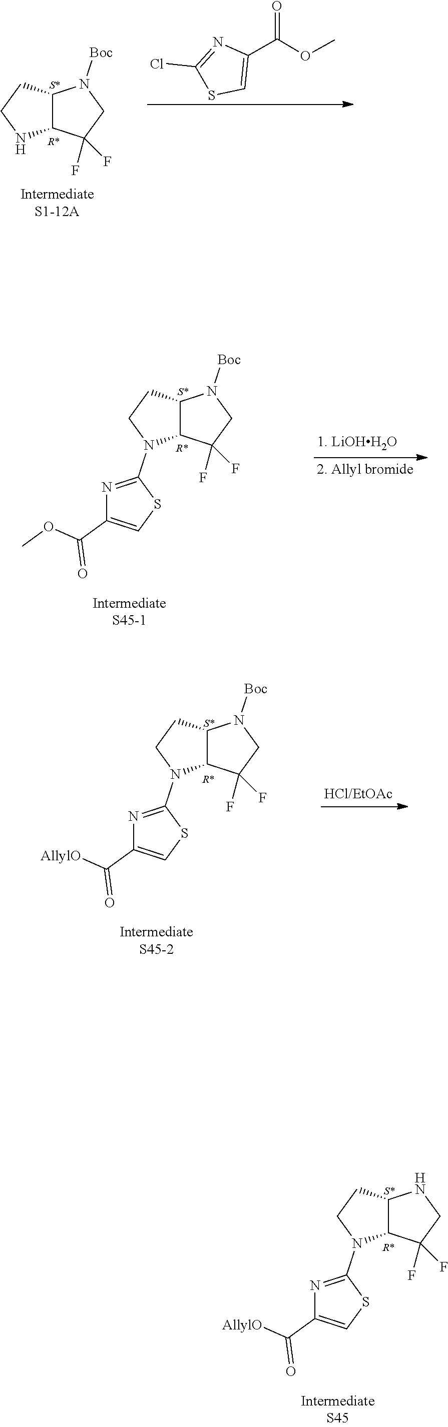

### S45-1: Methyl 2-((cis)-4-(tert-butoxycarbonyl)-6,6-difluorohexahydropyrrolo[3,2-b]pyrrol-1(2H)-yl)thiazole-4-carboxylate

To a solution of S1-12A (300 mg, 90% purity, 1.09 mmol) in N,N-dimethylformamide (3 mL) and N,N-diisopropylethylamine (0.5 mL) was added methyl 2-chlorothiazole-4-carboxylate (200 mg, 1.13 mmol). After stirred at 120° C. for 8 hours, the reaction mixture was cooled down to room temperature and purified C18 column (acetonitrile:water (+0.1% ammonium bicarbonate)=45% to 85%) to give the title compound (230 mg, 96% purity, 52% yield) as yellow solids. LC-MS (ESI): mass calcd. for C₁₆H₂₁F₂N₃O₄S 389.1, m/z found 390.3 [M+H]⁺.

### S45-2: Allyl 2-((cis)-4-(tert-butoxycarbonyl)-6,6-difluorohexahydropyrrolo[3,2-b]pyrrol-1(2H)-yl)thiazole-4-carboxylate

To a solution of S45-1 (230 mg, 96% purity, 0.567 mmol) in tetrahydrofuran (2 mL), methanol (0.5 mL) and water (0.5 mL) was added lithium hydroxide monohydrate (60 mg, 1.43 mmol) at room temperature. After stirred at room temperature for 6 hours, the mixture was concentrated and redissolved in N,N-dimethylformamide (3 mL). Potassium carbonate (160 mg, 1.16 mmol) and allyl bromide (700 mg, 5.79 mmol) were added and the mixture was stirred at room temperature overnight. Then it was purified C18 column (acetonitrile:water (+0.1% ammonium bicarbonate)=60% to 85%) to give the title compound (230 mg, 83% purity, 81% yield) as yellow solids. LC-MS (ESI): mass calcd. for C₁₈H₂₃F₂N₃O₄S 415.1, m/z found 416.4 [M+H]⁺.

### S45: Allyl 2-((cis)-6,6-difluorohexahydropyrrolo[3,2-b]pyrrol-1(2H)-yl)thiazole-4-carboxylate hydrochloride

To a solution of S45-2 (230 mg, 83% purity, 0.46 mmol) in ethyl acetate (2 mL) was added dropwise of 3.5 M hydrochloride in ethyl acetate (2 mL, 7 mmol). After stirred at room temperature overnight, the mixture was concentrated to give the title compound (150 mg, 79% purity, 73% yield) as white solids. LC-MS (ESI): mass calcd. for C₁₃H₁₆ClF₂N₃O₂S 351.1, m/z found 316.3 [M−HCl+H]⁺.

### Compound 124: 2-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)thiazole-4-carboxylic acid

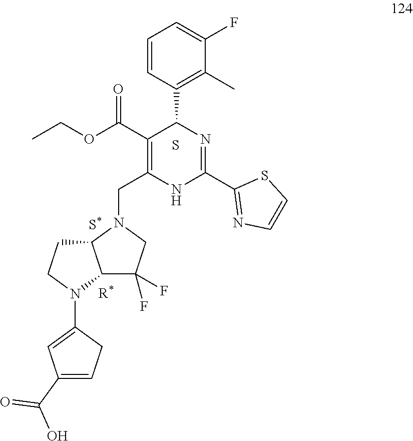

This compound was made from H2-1A and S45 according to typical method 1 and 2 successively. Purified by C18 column (acetonitrile:water (+0.1% ammonium bicarbonate)=35% to 60%) for two times to give the title compound (72 mg, 96.8% purity, 55% yield) as yellow solids. LC-MS (ESI): mass calcd. for C₂₈H₂₇F₃N₆O₄S₂ 632.2, m/z found 633.3 [M+H]⁺. ¹H NMR (400 MHz, CD₃OD) δ 7.92 (d, J=2.8 Hz, 1H), 7.74-7.72 (m, 2H), 7.20-7.14 (m, 2H), 6.99-6.94 (m, 1H), 6.01 (s, 1H), 4.72-4.65 (m, 1H), 4.40-4.35 (m, 1H), 4.18-4.02 (m, 4H), 3.92-3.81 (m, 2H), 3.43-3.40 (m, 1H), 3.13-3.01 (m, 1H), 2.53 (d, J=2.0 Hz, 3H), 2.31-2.23 (m, 1H), 2.14-2.02 (m, 1H), 1.15 (t, J=7.2 Hz, 3H).

### Compound 125: 4-((cis)-4-((6-(2-chloro-4-fluorophenyl)-5-(methoxycarbonyl)-2-(5-methyloxazol-4-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

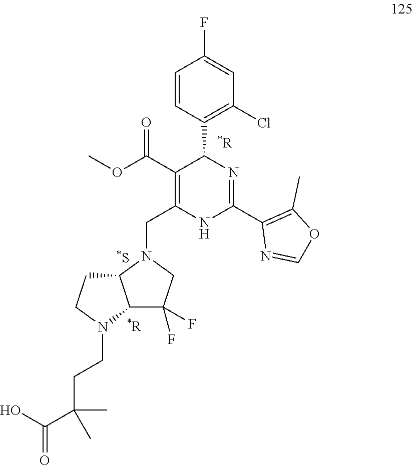 [SMILES: COC(=O)C1=C(CN2CC(F)(F)[C@H]3[C@@H]2CCN3CCC(C)(C)C(=O)O)NC(c2ncoc2C)=N[C@H]1c1ccc(F)cc1Cl]

This compound is made from H18-1A and S9 according to typical method 1 and 3. LC-MS (ESI): mass calcd. for C₂₉H₃₃ClF₃N₅O₅ 623.2, m/z found 624.2 [M+H]⁻. ¹H NMR (400 MHz, CD₃OD) δ 7.94 (s, 1H), 7.26-7.23 (m, 1H), 7.16 (dd, J=8.4, 2.0 Hz, 1H), 6.96 (td, J=8.4, 2.8 Hz, 1H), 6.04 (s, 1H), 4.17 (d, J=16.4 Hz, 1H), 4.06 (d, J=16.8 Hz, 1H), 3.81 (q, J=7.6 Hz, 1H), 3.53 (s, 3H), 3.31-3.14 (m, 3H), 2.99-2.91 (m, 1H), 2.81-2.74 (m, 1H), 2.54-2.48 (m, 1H), 2.44 (s, 3H), 2.42-2.38 (m, 1H), 1.92-1.82 (m, 2H), 1.74 (t, J=8.0 Hz, 2H), 1.13 (s, 6H).

### Compound 126: 4-((cis)-4-(((6-(2-Chloro-3,4-difluorophenyl)-5-(methoxycarbonyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

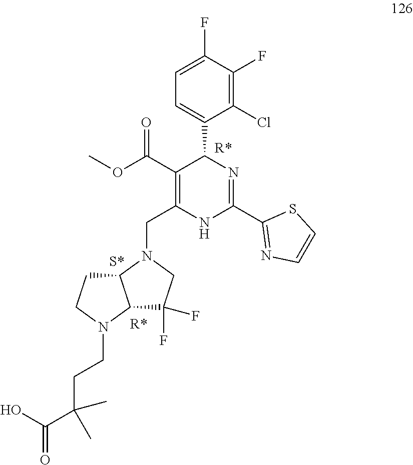 [SMILES: COC(=O)C1=C(CN2CC(F)(F)[C@H]3[C@@H]2CCN3CCC(C)(C)C(=O)O)NC(c2nccs2)=N[C@H]1c1ccc(F)c(F)c1Cl]

This compound was made from H5-1A and S9 according to typical method 1 and 3 successively. LC-MS (ESI): mass calcd. For C₂₈H₃₀ClF₄N₅O₄S 643.2, m/z found 644.1 [M+H]⁺. ¹H NMR (400 MHz, CDCl₃) δ 9.35 (br s, 1H), 7.86 (d, J=2.4 Hz, 1H), 7.44 (d, J=2.4 Hz, 1H), 7.04-7.00 (m, 2H), 6.18 (s, 1H), 4.25 (d, J=17.6 Hz, 1H), 4.04 (d, J=17.6 Hz, 1H), 3.86-3.79 (m, 1H), 3.59 (s, 3H), 3.49-3.29 (m, 3H), 3.19-3.09 (m, 1H), 3.00-2.89 (m, 1H), 2.72-2.63 (m, 1H), 2.54-2.44 (m, 1H), 2.10-1.87 (m, 3H), 1.69-1.63 (m, 1H), 1.28 (s, 3H), 1.27 (s, 3H).

### Compound 127: 4-((cis)-4-((6-(2-Chloro-4-fluorophenyl)-5-(methoxycarbonyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

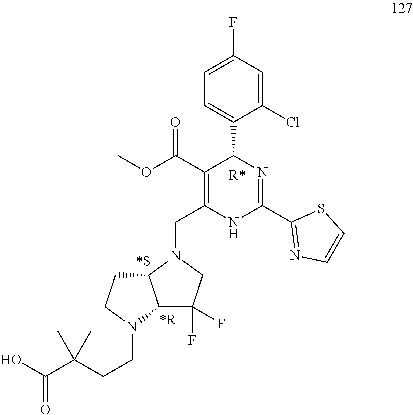 [SMILES: COC(=O)C1=C(CN2CC(F)(F)[C@H]3[C@@H]2CCN3CCC(C)(C)C(=O)O)NC(c2nccs2)=N[C@H]1c1ccc(F)cc1Cl]

This compound was made from H3-1A and S9 according to typical method 1 and 3 successively. LC-MS (ESI): mass calcd. For C₂₈H₃₁ClF₃N₅O₄S 625.2, m/z found 626.1 [M+H]⁺. ¹H NMR (400 MHz, CDCl₃) δ 9.33 (s, 1H), 7.86 (d, J=2.8 Hz, 1H), 7.43 (d, J=3.2 Hz, 1H), 7.28-7.24 (m, 1H), 7.14-7.11 (m, 1H), 6.94-6.89 (m, 1H), 6.18 (s, 1H), 4.26-4.22 (m, 1H), 4.06-4.02 (m, 1H), 3.85-3.80 (m, 1H), 3.59 (s, 3H), 3.44-3.31 (m, 3H), 3.14-3.07 (m, 1H), 2.99-2.91 (m, 1H), 2.70-2.64 (m, 1H), 2.52-2.46 (m, 1H), 2.04-1.89 (m, 3H), 1.71-1.65 (m, 1H), 1.28 (s, 3H), 1.27 (s, 3H).

### Compound 128: 4-((cis)-4-((6-(2-Chloro-3-fluorophenyl)-5-(methoxycarbonyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

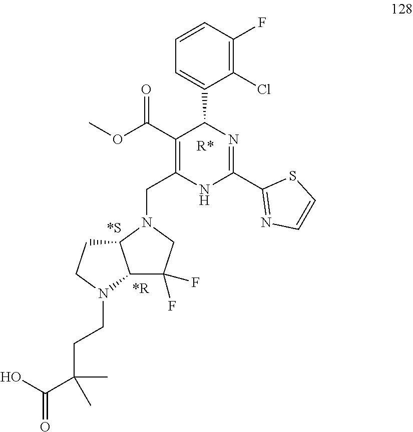 [SMILES: COC(=O)C1=C(CN2CC(F)(F)[C@H]3[C@@H]2CCN3CCC(C)(C)C(=O)O)NC(c2nccs2)=N[C@H]1c1cccc(F)c1Cl]

This compound was made from H11-A and S9 according to typical method 1 and 3 successively. LC-MS (ESI): mass calcd. for C₂₈H₃₁ClF₃N₅O₄S 625.2, m/z found 626.6 [M+H]⁺. ¹H NMR (400 MHz, CDCl₃) δ 9.35 (br s, 1H), 7.86 (d, J=3.6 Hz, 1H), 7.44 (d, J=1.6 Hz, 1H), 7.21-7.14 (m, 1H), 7.13-7.08 (m, 1H), 7.06-7.00 (m, 1H), 6.24 (s, 1H), 4.24 (d, J=17.2 Hz, 1H), 4.07 (d, J=18.4 Hz, 1H), 3.88-3.79 (m, 1H), 3.58 (s, 3H), 3.46-3.32 (m, 3H), 3.17-3.07 (m, 1H), 3.03-2.90 (m, 1H), 2.73-2.64 (m, 1H), 2.57-2.45 (m, 1H), 2.08-1.90 (m, 3H), 1.73-7.64 (m, 1H), 1.28 (s, 3H), 1.27 (s, 3H).

### Compound 129-M (S)-Ethyl 6-(((cis)-4-(2-(3-(tert-butoxycarbonyl)cyclobutyl)ethyl)-3,3-difluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate

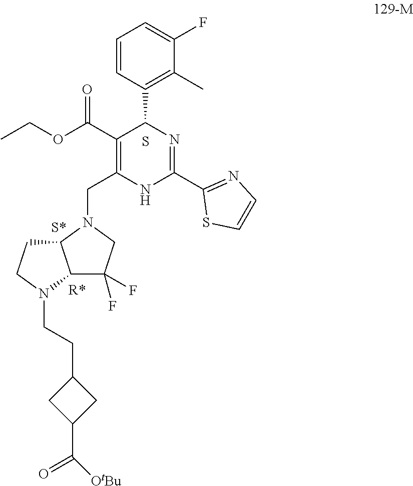

This compound was made from Compound 103 and tert-butyl 3-(2-oxoethyl)cyclobutanecarboxylate according typical method 5. LC-MS (ESI): mass calcd. for C₃₅H₄₄F₃N₅O₄S 687.8, m/z found 688.7 [M+H]⁺. ¹H NMR (400 MHz, CDCl₃) δ 9.36 (s, 1H), 7.83 (d, J=3.2 Hz, 1H), 7.40 (d, J=3.2 Hz, 1H), 7.10-7.04 (m, 1H), 6.99 (d, J=8.0 Hz, 1H), 6.92-6.88 (m, 1H), 6.00 (s, 1H), 4.22 (m, 2H), 4.08-3.97 (m, 2H), 3.85-3.79 (m, 1H), 3.36-3.14 (m, 3H), 3.04-2.83 (m, 3H), 2.54 (d, J=2.0 Hz, 3H), 2.45-2.24 (m, 5H), 1.95-1.82 (m, 4H), 1.73-1.64 (m, 2H), 1.45-1.44 (m, 9H), 1.11 (t, J=7.2 Hz, 3H).

Chiral separation: chiral Prep. HPLC (Column: Chiralpak IC 5 μm 20*250 mm; Mobile Phase: Hex:IPA:DEA=90:10:0.2 at 15 mL/min; Temp: 30° C.; Wavelength: 254 nm).

129-A: LC-MS (ESI): mass calcd. for C₃₅H₄₄F₃N₅O₄S 687.8, m/z found 688.3 [M+H]⁺. Chiral analysis (Column: Chiralpak IC 5 μm 4.6*250 mm; Mobile Phase: Hex:IPA:DEA=90:10:0.2 at 1.0 mL/min; Temp: 30° C.; Wavelength: 254 nm, R_{T}=9.404 min). ¹H NMR (300 MHz, CDCl₃) δ 9.36 (s, 1H), 7.82 (d, J=3.0 Hz, 1H), 7.40 (d, J=3.0 Hz, 1H), 7.11-7.04 (m, 1H), 6.99 (d, J=7.8 Hz, 1H), 6.93-6.87 (m, 1H), 6.00 (s, 1H), 4.23-4.09 (m, 2H), 4.06-3.98 (m, 2H), 3.86-3.78 (m, 1H), 3.37-3.13 (m, 3H), 3.02-2.82 (m, 2H), 2.75-2.66 (m, 1H), 2.54 (d, J=1.8 Hz, 3H), 2.46-2.22 (m, 5H), 1.98-1.82 (m, 4H), 1.66-1.57 (m, 2H), 1.44 (s, 9H), 1.12 (t, J=7.2 Hz, 3H).

129-B: LC-MS (ESI): mass calcd. for C₃₅H₄₄F₃N₅O₄S 687.8, m/z found 688.3 [M+H]⁺. Chiral analysis (Column: Chiralpak IC 5 μm 4.6*250 mm; Mobile Phase: Hex:IPA:DEA=90:10:0.2 at 1.0 mL/min; Temp: 30° C.; Wavelength: 254 nm, R_{T}=10.444 min). ¹H NMR (400 MHz, CDCl₃) δ 9.36 (s, 1H), 7.83 (d, J=3.2 Hz, 1H), 7.40 (d, J=2.8 Hz, 1H), 7.10-7.04 (m, 1H), 6.99 (d, J=6.8 Hz, 1H), 6.92-6.88 (m, 1H), 6.00 (s, 1H), 4.16 (t, J=17.2 Hz, 2H), 4.08-3.99 (m, 2H), 3.85-3.80 (m, 1H), 3.35-3.16 (m, 3H), 3.03-2.94 (m, 2H), 2.74-2.67 (m, 1H), 2.54 (d, J=1.6 Hz, 3H), 2.48-2.33 (m, 5H), 1.97-1.84 (m, 4H), 1.71-1.66 (m, 2H), 1.45 (s, 9H), 1.11 (t, J=7.2 Hz, 3H).

### Compound 129A and 129B: 3-(2-((cis)-4-(((S)-5-(Ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)ethyl)cyclobutanecarboxylic acid

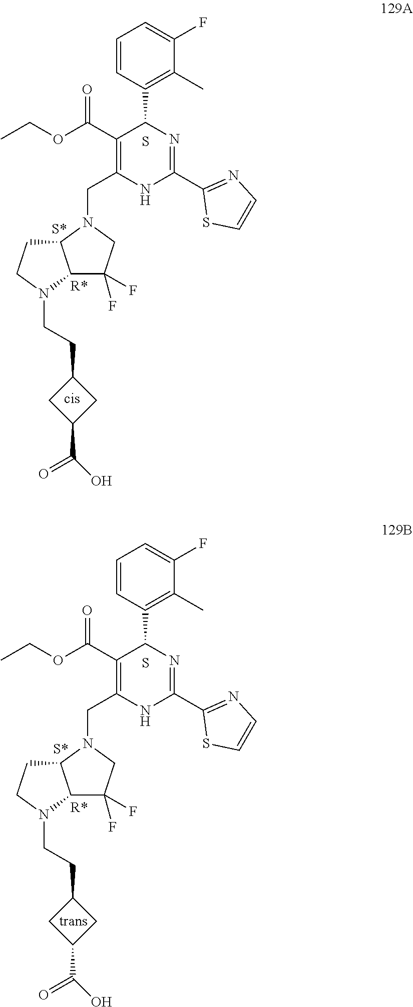

Compound 129A and 129B were made from 129-A and 129-B according to typical method 3, respectively.

129A: LC-MS (ESI): mass calcd. for C₃₁H₃₆F₃N₅O₄S 631.2, m/z found 632.4 [M+H]⁺. ¹H NMR (400 MHz, CDCl₃) δ 9.35 (s, 1H), 7.82 (d, J=3.2 Hz, 1H), 7.40 (d, J=2.8 Hz, 1H), 7.10-7.04 (m, 1H), 6.99 (d, J=7.2 Hz, 1H), 6.92-6.88 (m, 1H), 6.00 (s, 1H), 4.16 (q, J=17.6 Hz, 2H), 4.08-3.97 (m, 2H), 3.85-3.80 (m, 1H), 3.35-3.16 (m, 3H), 3.07-2.93 (m, 2H), 2.75-2.68 (m, 1H), 2.54 (d, J=2.0 Hz, 3H), 2.48-2.44 (m, 1H), 2.43-2.31 (m, 4H), 2.00-1.90 (m, 4H), 1.70-1.58 (m, 2H), 1.11 (t, J=7.2 Hz, 3H).

129B: LC-MS (ESI): mass calcd. for C₃₁H₃₆F₃N₅O₄S 631.2, m/z found 631.9 [M+H]⁺. ¹H NMR (400 MHz, CDCl₃) δ 9.35 (s, 1H), 7.81 (d, J=3.2 Hz, 1H), 7.38 (d, J=3.2 Hz, 1H), 7.09-7.04 (m, 1H), 6.99-6.98 (m, 1H), 6.89 (t, J=8.4 Hz, 1H), 6.00 (s, 1H), 4.20 (d, J=17.6 Hz, 1H), 4.11 (d, J=17.6 Hz, 1H), 4.08-3.98 (m, 2H), 3.85-3.79 (m, 1H), 3.35-3.10 (m, 4H), 3.00-2.93 (m, 1H), 2.74-2.70 (m, 1H), 2.53 (d, J=2.0 Hz, 3H), 2.47-2.38 (m, 5H), 1.99-1.88 (m, 4H), 1.74-1.65 (m, 2H), 1.11 (t, J=7.2 Hz, 3H).

### Preparation of Intermediate S42

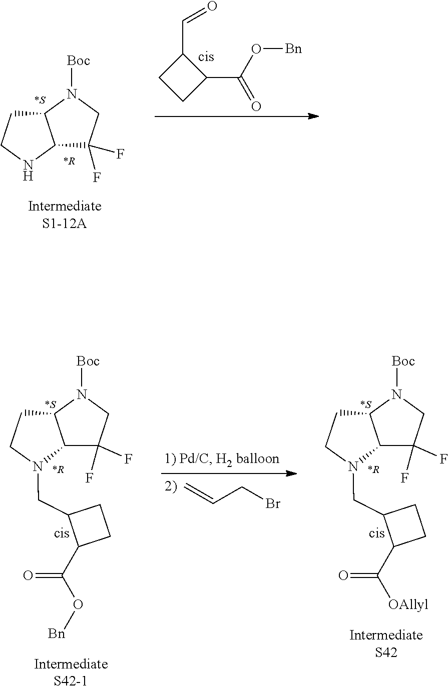

### S42-1: (cis, cis)-tert-Butyl 4-((2-((benzyloxy)carbonyl)cyclobutyl)-methyl)-3,3-difluorohexahydropyrrolo[3,2-b]pyrrole-1(2H)-carboxylate

This intermediate was made from S1-12A and (cis)-benzyl 2-formylcyclobutanecarboxylate according to typical method 5. ¹H NMR (400 MHz, CDCl₃) δ 7.34-7.28 (m, 5H), 5.15-5.02 (m, 2H), 4.47-4.26 (m, 1H), 3.87-3.49 (m, 2H), 3.14-2.70 (m, 5H), 2.36-1.97 (m, 5H), 1.76-1.57 (m, 3H), 1.46 (s, 9H).

### S42: (cis, cis)-tert-Butyl 4-((2-((allyloxy)carbonyl)cyclobutyl)methyl)-3,3-difluorohexahydropyrrolo[3,2-b]pyrrole-1(2H)-carboxylate

To a solution of S42-1 (200 mg, 90% purity, 0.400 mmol) in ethanol (5 mL) was 10% palladium on activated carbon wt. (40 mg, 0.038 mmol) at room temperature. After stirred under a hydrogen of balloon at room temperature for 1 hour, the reaction mixture was filtered and concentrated to afford a residue, which was diluted in N,N-dimethylformamide (5 mL). To this solution was added potassium carbonate (165 mg, 1.19 mmol) and allyl bromide (100 mg, 0.827 mmol). After stirred at 25° C. for 3 hours, the mixture was purified by C18 (acetonitrile:water=60% to 75%) to give the title compound (120 mg, 80% purity, 60% yield) as yellow oil. LC-MS (ESI): R_{T}=1.96 min, mass calcd. for C₂₀H₃₀F₂N₂O₄ 400.5, m/z found 401.4 [M+H]⁺.

### Compound 130A and 130B: (cis)-2-(((cis)-4-(((S)-5-(Ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)methyl)cyclobutanecarboxylic acid

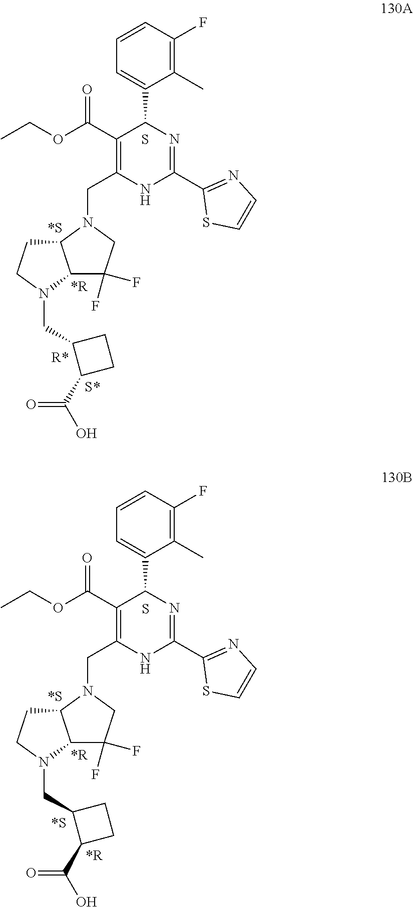

130A and 130B were made from H2-1A and S42 according to typical method 1 and typical method 2 successively.

130A: purified by C18 column (acetonitrile:water (40% to 50%) to give the title compound (10 mg, 94.1% purity, 41% yield) as yellow solids. LC-MS (ESI): mass calcd. for C₃₀H₃₄F₃N₅O₄S 617.7, m/z found 618.3 [M+H]⁺. ¹H NMR (400 MHz, CD₃OD) δ 7.89 (d, J=2.8 Hz, 1H), 7.69 (d, J=3.2 Hz, 1H), 7.16-7.08 (m, 2H), 6.95-6.90 (m, 1H), 5.95 (s, 1H), 4.24-4.11 (m, 2H), 4.05 (q, J=7.2 Hz, 2H), 3.93-3.87 (m, 1H), 3.37-3.31 (m, 3H), 3.08-2.97 (m, 1H), 2.94-2.84 (m, 2H), 2.78-2.66 (m, 2H), 2.50 (s, 3H), 2.47-2.42 (m, 1H), 2.11-1.91 (m, 5H), 1.70-1.65 (m, 1H), 1.12 (t, J=7.2 Hz, 3H).

130B: purified by Pre. HPLC (Column: Xbridge C18 (5 μm 19*150 mm), Mobile Phase A: water (0.1% ammonium bicarbonate), Mobile Phase B: acetonitrile, UV: 254 nm, Flow rate: 15 mL/min, Gradient: 25-90% (% B)) to give the title compound (4.3 mg, 98.2% purity, 20% yield) as yellow solids. LC-MS (ESI): mass calcd. for C₃₀H₃₄F₃N₅O₄S 617.7, m/z found 618.3 [M+H]⁺. ¹H NMR (400 MHz, CD₃OD) δ 7.90 (d, J=3.2 Hz, 1H), 7.70 (d, J=3.2 Hz, 1H), 7.15-7.09 (m, 2H), 6.95-6.90 (m, 1H), 5.96 (s, 1H), 4.26-4.10 (m, 2H), 4.04 (q, J=7.2 Hz, 2H), 3.90-3.84 (m, 1H), 3.39-3.37 (m, 1H), 3.28-3.17 (m, 2H), 3.04-2.91 (m, 2H), 2.79-2.75 (m, 3H), 2.58-2.54 (m, 1H), 2.50 (s, 3H), 2.17-2.08 (m, 3H), 1.97-1.87 (m, 2H), 1.76-1.66 (m, 1H), 1.12 (t, J=7.2 Hz, 3H).

### Compound 131A and 131B: 3-((cis)-6,6-Difluoro-4-(((S*)-6-(3-fluoro-2-methylphenyl)-5-(methoxycarbonyl)-2-(thiazol-2-yl)-3,6-dihydropyrimidin-4-yl)methyl)hexahydropyrrolo[3,2-b]pyrrol-1(2H)-yl)-2-methylpropanoic acid (Single Diastereomers)

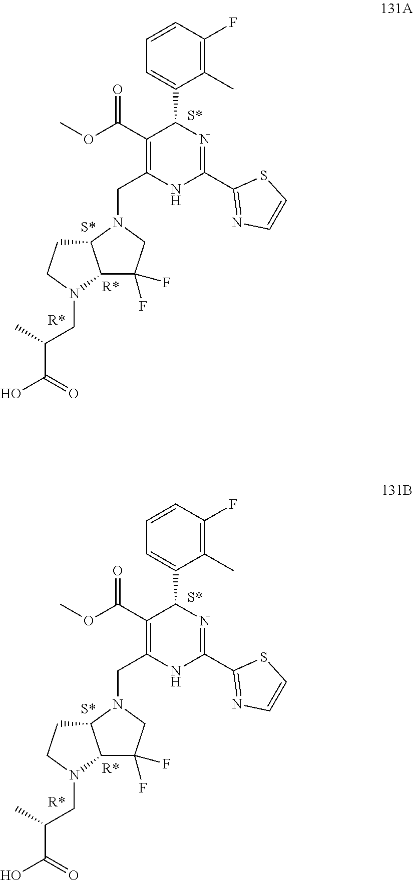 [SMILES: COC(=O)C1=C(CN2CC(F)(F)[C@H]3[C@@H]2CCN3C[C@@H](C)C(=O)O)NC(c2nccs2)=N[C@H]1c1cccc(F)c1C ; COC(=O)C1=C(CN2CC(F)(F)[C@H]3[C@@H]2CCN3C[C@@H](C)C(=O)O)NC(c2nccs2)=N[C@H]1c1cccc(F)c1C]

### Preparation of Intermediate S43A and Intermediate S43B

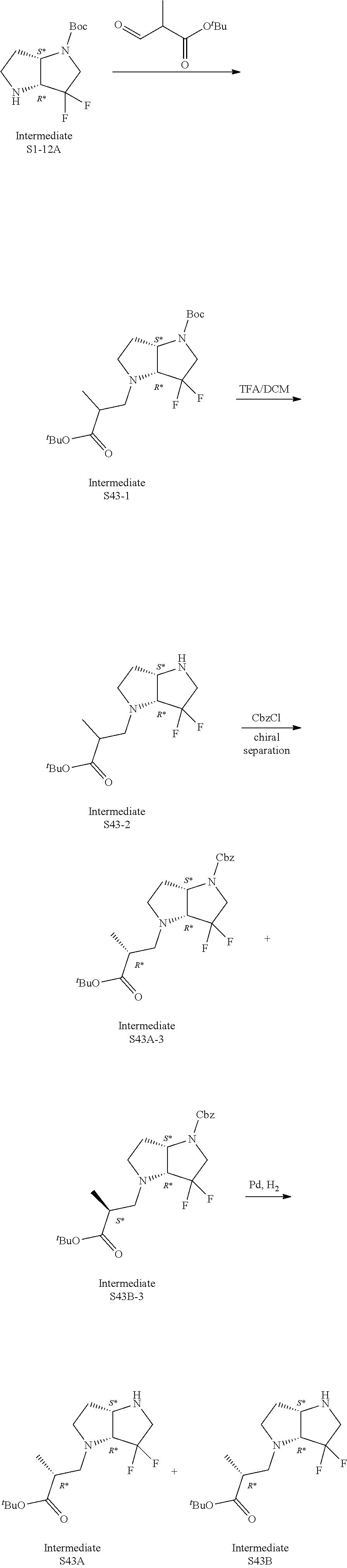

### S43-1: (cis)-tert-Butyl 4-(3-(tert-butoxy)-2-methyl-3-oxopropyl)-3,3-difluorohexahydropyrrolo[3,2-b]pyrrole-1(2H)-carboxylate

This intermediate is made from S1-12A and tert-butyl 2-methyl-3-oxopropanoate according to typical method 2. ¹H NMR (400 MHz, CDCl₃) δ 4.50-4.34 (m, 1H), 3.90-3.52 (m, 2H), 3.90-2.82 (m, 3H), 2.71-2.20 (m, 4H), 1.91-1.76 (m, 1H), 1.46 (s, 9H), 1.44 (s, 9H), 1.14 (d, J=6.8 Hz, 1.7H), 1.09 (d, J=6.8 Hz, 1.3H).

### S43-2: tert-Butyl 3-((cis)-6,6-difluorohexahydropyrrolo[3,2-b]pyrrol-1(2H)-yl)-2-methylpropanoate

To a solution of S43-1 (3.40 g, 90% purity, 7.84 mmol) in dichloromethane (35 mL) was added trifluoroacetic acid (5 mL) at room temperature. After stirred at room temperature for 5 hours, the mixture was basidified with cold saturated aqueous sodium carbonate until pH=8 and the organic layer was separated. The aqueous layer was extracted with dichloromethane (150 mL) twice. The combined organic layers were washed with brine (200 mL), dried over Na₂SO₄₍ₛ₎ and filtered. The filtrate was concentrated to give the desired compound (2.28 g, 80% purity from ¹H NMR, 80% yield) as brown oil. ¹H NMR (300 MHz, CDCl₃) δ 4.58-3.70 (m, 1H), 3.47-2.88 (m, 4H), 2.83-2.77 (m, 1H), 2.72-2.55 (m, 1H), 2.50-2.10 (m, 3H), 2.00-1.71 (m, 1H), 1.56 (s, 9H), 1.27 (d, J=6.9 Hz, 2.3H), 1.24 (d, J=6.9 Hz, 0.7H).

### S43A-3 and S43B-3: (cis)-Benzyl 4-(3-(tert-butoxy)-2-methyl-3-oxopropyl)-3,3-difluorohexahydropyrrolo[3,2-b]pyrrole-1(2H)-carboxylate

To a solution of S43-2 (2.27 g, 80% purity, 6.26 mmol) in acetonitrile (25 mL) and water (25 mL) at 5° C. was added sodium carbonate (1.66 g, 15.7 mmol) and benzyl carbonochloridate (1.60 g, 9.38 mmol). After stirred at room temperature overnight, the mixture was poured into water (100 mL) and extracted with ethyl acetate (100 mL) for three times. The combined organic phases were washed with brine (200 mL), dried over Na₂SO₄₍ₛ₎ and filtered. The filtrate was concentrated in vacuum to give a residue, which was purified by column C18 (acetonitrile:water (+0.02% ammonium bicarbonate)=05% to 90%) to give a colorless oil, which was separated by chiral HPLC (Column: Chiralpak IE 5 μm 20*250 mm; Mobile Phase: Hex:EtOH=80:20 at 5 mL/min; Temp: 30° C.; Wavelength: 214 nm) to give the desired products S43A-3 (1.42 g, 100% purity, 55% yield, 100% stereopure) and S43B-3 (272 mg, 96% purity, 10% yield, 99.8% stereopure) as white solids.

S43A-3: LC-MS (ESI): mass calcd. for C₂₂H₃₀F₂N₂O₄ 424.2, m/z found 425.5 [M+H]⁺. Chiral analysis (Column: Chiralpak IE 5 μm 4.6*250 mm; Mobile Phase: Hex:EtOH=80:20 at 1 mL/min; Temp: 30° C.; Wavelength: 214 nm, R_{T}=5.690 min). ¹H NMR (400 MHz, CDCl₃) δ 7.39-7.29 (m, 5H), 5.20-5.08 (m, 2H), 4.55-4.45 (m, 1H), 3.98-3.84 (m, 1H), 3.76-3.64 (m, 1H), 3.21-3.10 (m, 2H), 2.87-2.82 (m, 1H), 2.72-2.65 (m, 1H), 2.54-2.45 (m, 1H), 2.41-2.17 (m, 2H), 1.97-1.79 (m, 1H), 1.44 (s, 9H), 1.14 (d, J=7.2 Hz, 3H).

S43B-3: LC-MS (ESI): mass calcd. for C₂₂H₃₀F₂N₂O₄ 424.2, m/z found 425.5 [M+H]⁺. Chiral analysis (Column: Chiralpak IE 5 μm 4.6*250 mm; Mobile Phase: Hex:EtOH=80:20 at 1 mL/min; Temp: 30° C.; Wavelength: 214 nm, R_{T}=7.293 min). ¹H NMR (400 MHz, CDCl₃) δ 7.43-7.29 (m, 5H), 5.20-5.08 (m, 2H), 4.54-4.44 (m, 1H), 3.97-3.83 (m, 1H), 3.70-3.58 (m, 1H), 3.40-3.33 (m, 1H), 3.16 (t, J=11.2 Hz, 1H), 3.08-3.02 (m, 1H), 2.59-2.48 (m, 1H), 2.38-2.18 (m, 3H), 1.92-1.79 (m, 1H), 1.42 (s, 9H), 1.09 (d, J=6.8 Hz, 3H).

### S43A: tert-Butyl 3-((cis)-6,6-difluorohexahydropyrrolo[3,2-b]pyrrol-1(2H)-yl)-2-methylpropanoate

To a mixture of S43A-3 (1.40 g, 100% purity, 3.43 mmol) in isopropanol (20 mL) was added palladium diacetate (350 mg, 1.56 mmol) and activated carbon (150 mg) at room temperature. The mixture was stirred at 50° C. under hydrogen atmosphere (1 atm) for 3 hours. Then it was filtered and the filtrate was concentrated in vacuum to give a residue, which was diluted with dichloromethane (30 mL) and water (20 mL). The aqueous layer was separated and extracted with dichloromethane (30 mL) twice. The combined organic layers were washed with saturated sodium bicarbonate aqueous solution (50 mL), dried over Na₂SO₄₍ₛ₎ and filtered. The filtrate was concentrated to give the desired compound (937 mg, 95% purity, 89% yield) as colorless oil. LC-MS (ESI): mass calcd. for C₁₄H₂₄F₂N₂O₂ 290.2, m/z found 291.5 [M+H]⁺. ¹H NMR (400 MHz, CDCl₃) δ 3.98 (q, J=7.6 Hz, 1H), 3.19-2.96 (m, 3H), 2.90-2.77 (m, 2H), 2.71-2.66 (m, 1H), 2.57-2.48 (m, 1H), 2.25-2.11 (m, 2H), 1.73-1.60 (m, 1H), 1.44 (s, 9H), 1.15 (d, J=7.2 Hz, 3H).

Analogously, S43B was prepared. LC-MS (ESI): mass calcd. for C₁₄H₂₄F₂N₂O₂ 290.2, m/z found 291.4 [M+H]⁺. ¹H NMR (400 MHz, CDCl₃) δ 3.98 (q, J=8.0 Hz, 1H), 3.28 (t, J=8.0 Hz, 1H), 3.14-3.03 (m, 2H), 2.98 (t, J=14.0 Hz, 1H), 2.78 (q, J=8.0 Hz, 1H), 2.60-2.50 (m, 1H), 2.33 (dd, J=12.0, 4.8 Hz, 1H), 2.17-2.10 (m, 2H), 1.68-1.59 (m, 1H), 1.09 (d, J=6.8 Hz, 3H).

131A was made from H4-1B and S43A according to typical method 1 and typical method 3 successively. Purified by column C18 (acetonitrile:water (+0.02% ammonium bicarbonate)=5% to 55%) to give the desired product (33 mg, 96% purity, 52% yield) as yellow solids. LC-MS (ESI): mass calcd. for C₂₇H₃₀F₃N₅O₄S 577.2, m/z found 578.2 [M+H]⁺. ¹H NMR (400 MHz, CDCl₃): δ 9.25 (br s, 1H), 7.83 (d, J=2.8 Hz, 1H), 7.42 (d, J=2.8 Hz, 1H), 7.09-7.04 (m, 1H), 6.97-6.88 (m, 2H), 6.01 (s, 1H), 4.36 (d, J=17.6 Hz, 1H), 4.05 (d, J=17.6 Hz, 1H), 3.86-3.80 (m, 1H), 3.74-3.63 (m, 1H), 3.60 (s, 3H), 3.36-3.25 (m, 2H), 3.08-2.89 (m, 4H), 2.64-2.56 (m, 1H), 2.54 (d, J=2.0 Hz, 3H), 2.06-1.94 (m, 2H), 1.21 (d, J=7.2 Hz, 3H).

131B was made from H4-1B and S43B according to typical method 1 and typical method 3 successively. Purified by column C18 (acetonitrile:water (+0.02% ammonium bicarbonate)=5% to 55%) to give the desired product (31 mg, 99% purity, 81% yield) as yellow solids. LC-MS (ESI): R_{T}=3.277 min, mass calcd. for C₂₇H₃₀F₃N₅O₄S 577.2, m/z found 577.9 [M+H]⁺. ¹H NMR (400 MHz, CDCl₃) δ 9.32 (br s, 1H), 7.88 (d, J=2.8 Hz, 1H), 7.42 (d, J=2.8 Hz, 1H), 7.09-7.04 (m, 1H), 6.97-6.88 (m, 2H), 6.00 (s, 1H), 4.37 (d, J=17.6 Hz, 1H), 4.08 (d, J=17.6 Hz, 1H), 3.91-3.86 (m, 1H), 3.60 (s, 3H), 3.584-3.51 (m, 1H), 3.47-3.35 (m, 2H), 3.09-2.98 (m, 2H), 2.77 (dd, J=12.0, 4.8 Hz, 1H), 2.70-2.59 (m, 2H), 2.54 (d, J=1.2 Hz, 3H), 2.19-2.10 (m, 1H), 2.08-1.98 (m, 1H), 1.23 (d, J=7.2 Hz, 3H).

### Compound 132A and 132B: 3-((cis)-4-((6-(2-Chloro-3-fluorophenyl)-5-(methoxycarbonyl)-2-(thiazol-2-yl)-3,6-dihydropyrimidin-4-yl)methyl)-6,6-difluorohexahydropyrrolo[3,2-b]pyrrol-1(2H)-yl)-2-methylpropanoic acid (Single Diastereomers)

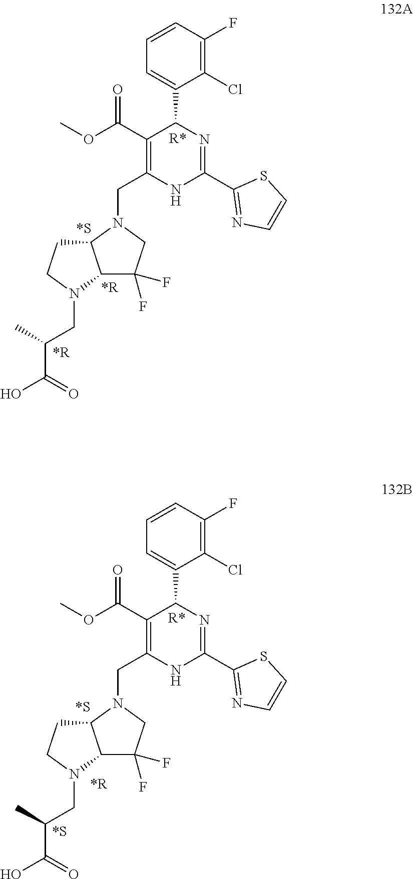 [SMILES: COC(=O)C1=C(CN2CC(F)(F)[C@H]3[C@@H]2CCN3C[C@@H](C)C(=O)O)NC(c2nccs2)=N[C@H]1c1cccc(F)c1Cl ; COC(=O)C1=C(CN2CC(F)(F)[C@H]3[C@@H]2CCN3C[C@H](C)C(=O)O)NC(c2nccs2)=N[C@H]1c1cccc(F)c1Cl]

132A was made from H11-A and S43A according to typical method 1 and typical method 3 successively. LC-MS (ESI): mass calcd. for C₂₆H₂₇ClF₃N₅O₄S 597.1, m/z found 597.9 [M+H]⁺. ¹H NMR (400 MHz, CDCl₃) δ 9.32 (br s, 1H), 7.86 (d, J=3.2 Hz, 1H), 7.44 (d, J=2.8 Hz, 1H), 7.19-7.14 (m, 1H), 7.13-7.08 (m, 1H), 7.07-7.01 (m, 1H), 6.26 (s, 1H), 4.34 (d, J=17.6 Hz, 1H), 4.02 (d, J=17.2 Hz, 1H), 3.88-3.81 (m, 1H), 3.71-3.62 (m, 1H), 3.59 (s, 3H), 3.37-3.26 (m, 2H), 3.07-2.90 (m, 4H), 2.66-2.56 (m, 1H), 2.03-1.95 (m, 2H), 1.21 (d, J=7.2 Hz, 3H).

132B was made analogously: LC-MS (ESI): mass calcd. for C₂₆H₂₇ClF₃N₅O₄S 597.1, m/z found 597.8 [M+H]⁺. ¹H NMR (400 MHz, CDCl₃) δ 9.37 (br s, 1H), 7.91 (d, J=2.8 Hz, 1H), 7.44 (d, J=3.2 Hz, 1H), 7.20-7.14 (m, 1H), 7.13-7.08 (m, 1H), 7.07-7.01 (m, 1H), 6.25 (s, 1H), 4.27 (d, J=17.2 Hz, 1H), 4.05 (d, J=17.2 Hz, 1H), 3.92-3.84 (m, 1H), 3.61-3.53 (m, 41H), 3.46-3.35 (m, 2H), 3.09-2.98 (m, 2H), 2.81-2.73 (m, 1H), 2.70-2.58 (m, 2H), 2.19-2.11 (m, 1H), 2.07-1.99 (m, 1H), 1.24 (d, J=6.8 Hz, 3H).

### Compound 133A: 3-((cis)-4-((6-(2-Chloro-4-fluorophenyl)-5-(methoxycarbonyl)-2-(thiazol-2-yl)-3,6-dihydropyrimidin-4-yl)methyl)-6,6-difluorohexahydropyrrolo[3,2-b]pyrrol-1(2H)-yl)-2-methylpropanoic acid (Single Diastereomer)

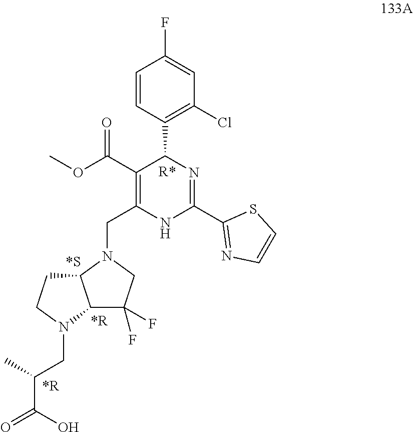 [SMILES: COC(=O)C1=C(CN2CC(F)(F)[C@H]3[C@@H]2CCN3C[C@@H](C)C(=O)O)NC(c2nccs2)=N[C@H]1c1ccc(F)cc1Cl]

This compound was made from H3-1A and S43A according to typical method 1 and typical method 3 successively. LC-MS (ESI): mass calcd. for C₂₆H₂₇ClF₃N₅O₄S 597.1, m/z found 598.2 [M+H]⁺. ¹H NMR (400 MHz, CDCl₃) δ 9.23 (br s, 1H), 7.86 (d, J=3.2 Hz, 1H), 7.45 (d, J=2.7 Hz, 1H), 7.29-7.27 (m, 1H), 7.14 (dd, J=8.8, 2.8 Hz, 1H), 6.94-6.89 (m, 1H), 6.19 (s, 1H), 4.32 (d, J=16.8 Hz, 1H), 4.00 (d, J=17.2 Hz, 1H), 3.87-3.82 (m, 1H), 3.74-3.65 (m, 1H), 3.60 (s, 3H), 3.29-3.28 (m, 2H), 3.10-2.89 (m, 4H), 2.67-2.51 (m, 2H), 2.07-1.92 (m, 2H), 1.21 (d, J=6.8 Hz, 3H).

### Compound 134A and 134B: 3-((cis)-4-((6-(2-chloro-3,4-difluorophenyl)-5-(methoxycarbonyl)-2-(thiazol-2-yl)-3,6-dihydropyrimidin-4-yl)methyl)-6,6-difluorohexahydropyrrolo[3,2-b]pyrrol-1(2H)-yl)-2-methylpropanoic acid (Single Diastereomers)

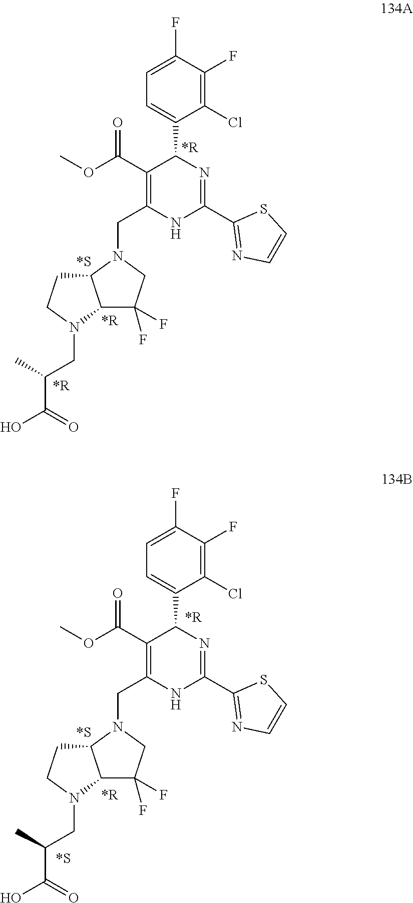 [SMILES: COC(=O)C1=C(CN2CC(F)(F)[C@H]3[C@@H]2CCN3C[C@@H](C)C(=O)O)NC(c2nccs2)=N[C@H]1c1ccc(F)c(F)c1Cl ; COC(=O)C1=C(CN2CC(F)(F)[C@H]3[C@@H]2CCN3C[C@H](C)C(=O)O)NC(c2nccs2)=N[C@H]1c1ccc(F)c(F)c1Cl]

134A was made from H5-1A and S43A according to typical method 1 and typical method 3 successively. LC-MS (ESI): mass calcd. For C₂₆H₂₆ClF₄N₅O₄S 615.1, m/z found 616.2 [M+H]⁺. ¹H NMR (400 MHz, CDCl₃) δ 9.35 (br s, 1H), 7.86 (d, J=3.2 Hz, 1H), 7.46 (d, J=3.2 Hz, 1H), 7.07-7.00 (m, 2H), 6.19 (s, 1H), 4.35-4.31 (m, 1H), 4.03-3.99 (m, 1H), 3.87-3.83 (m, 1H), 3.74-3.63 (m, 1H), 3.60 (s, 3H), 3.37-3.26 (m, 2H), 3.07-2.93 (m, 4H), 2.66-2.57 (m, 1H), 2.02-1.97 (m, 2H), 1.21 (d, J=6.8 Hz, 3H).

134B was made analogously. LC-MS (ESI): mass calcd. For C₂₆H₂₆ClF₄N₅O₄S 615.1, m/z found 616.2 [M+H]⁺. ¹H NMR (400 MHz, CDCl₃) δ 7.91 (d, J=3.2 Hz, 1H), 7.46 (d, J=2.8 Hz, 1H), 7.07-7.00 (m, 2H), 6.18 (s, 1H), 4.29-4.24 (m, 1H), 4.07-4.03 (m, 1H), 3.91-3.86 (m, 1H), 3.59 (s, 3H), 3.57-3.52 (m, 1H), 3.47-3.37 (m, 2H), 3.10-2.99 (m, 2H), 2.79-2.75 (m, 1H), 2.71-2.60 (m, 2H), 2.19-2.11 (m, 1H), 2.08-2.01 (m, 1H), 1.24 (d, J=6.8 Hz, 3H).

### Compound 135A and 135B: 3-((cis)-4-((6-(2-chloro-4-fluorophenyl)-5-(ethoxycarbonyl)-2-(thiazol-2-yl)-3,6-dihydropyrimidin-4-yl)methyl)-6,6-difluorohexahydropyrrolo[3,2-b]pyrrol-1(2H)-yl)-2-methylpropanoic acid

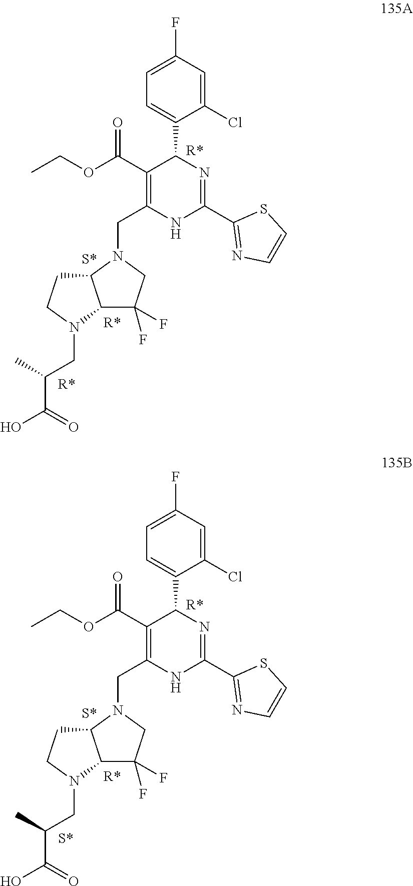 [SMILES: CCOC(=O)C1=C(CN2CC(F)(F)[C@H]3[C@@H]2CCN3C[C@@H](C)C(=O)O)NC(c2nccs2)=N[C@H]1c1ccc(F)cc1Cl ; CCOC(=O)C1=C(CN2CC(F)(F)[C@H]3[C@@H]2CCN3C[C@H](C)C(=O)O)NC(c2nccs2)=N[C@H]1c1ccc(F)cc1Cl]

135A was made from H12-1A and S43A according to typical method 1 and typical method 3 successively. LC-MS (ESI): mass calcd. for C₂₇H₂₉ClF₃N₅O₄S 611.1, m/z found 611.9 [M+H]⁺. ¹H NMR (400 MHz, CDCl₃) δ 7.87 (d, J=3.2 Hz, 1H), 7.46 (d, J=3.2 Hz, 1H), 7.31-7.28 (m, 1H), 7.15-7.12 (m, 1H), 6.95-6.90 (m, 1H), 6.21 (s, 1H), 4.35-4.31 (m, 1H), 4.03 (q, J=7.2 Hz, 3H), 3.88-3.84 (m, 1H), 3.72-3.64 (m, 1H), 3.36-3.30 (m, 2H), 3.08-2.94 (m, 4H), 2.67-2.57 (m, 1H), 2.04-1.97 (m, 2H), 1.21 (d, J=7.2 Hz, 3H), 1.12 (t, J=7.2 Hz, 3H).

135B was made analogously. LC-MS (ESI): mass calcd. for C₂₇H₂₉ClF₃N₅O₄S 611.1, m/z found 612.2 [M+H]⁺. ¹H NMR (400 MHz, CDCl₃) δ 9.32 (s, 1H), 7.90 (d, J=3.2 Hz, 1H), 7.43 (d, J=2.8 Hz, 1H), 7.30-7.28 (m, 1H), 7.14-7.11 (m, 1H), 6.94-6.89 (m, 1H), 6.21 (s, 1H), 4.29-4.25 (m, 1H), 4.06-4.00 (m, 3H), 3.90-3.85 (m, 1H), 3.59-3.54 (m, 1H), 3.46-3.35 (m, 2H), 3.09-2.97 (m, 2H), 2.79-2.75 (m, 1H), 2.69-2.59 (m, 2H), 2.18-1.98 (m, 2H), 1.23 (d, J=7.2 Hz, 3H), 1.12 (t, J=7.2 Hz, 3H).

### Compound 136: 4-((cis)-6,6-difluoro-4-((6-(3-fluoro-2-methylphenyl)-5-(methoxycarbonyl)-2-(thiazol-2-yl)-3,6-dihydropyrimidin-4-yl)methyl)hexahydropyrrolo[3,2-b]pyrrol-1(2H)-yl)-2,2-dimethylbutanoic acid (Single Diastereomer)

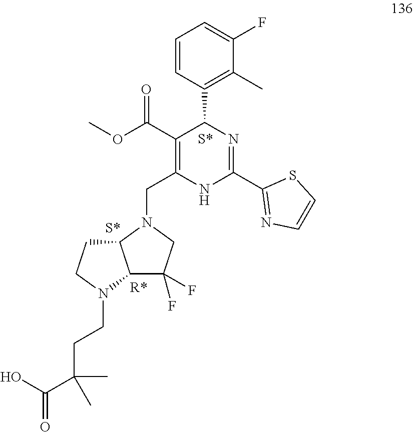 [SMILES: COC(=O)C1=C(CN2CC(F)(F)[C@H]3[C@@H]2CCN3CCC(C)(C)C(=O)O)NC(c2nccs2)=N[C@H]1c1cccc(F)c1C]

This compound was made from S9 and H4-1B according to typical method 1 and typical method 3. Purified by C18 column (acetonitrile:water (+0.02% ammonium bicarbonate)=40% to 55%) to give the title compound (30.8 mg, 98.6% purity, 57% yield) as yellow solids. LC-MS (ESI): mass calcd. for C₂₉H₃₄F₃N₅O₄S 605.2, m/z found 605.9 [M+H]⁺. ¹H NMR (400 MHz, CDCl₃) δ 9.28 (s, 1H), 7.84 (d, J=3.2 Hz, 1H), 7.41 (d, J=2.8 Hz, 1H), 7.10-7.04 (m, 1H), 6.97-6.88 (m, 2H), 5.99 (s, 1H), 4.26 (d, J=17.2 Hz, 1H), 4.10 (d, J=17.6 Hz, 1H), 3.86-3.82 (m, 1H), 3.59 (s, 3H), 3.46-3.33 (m, 3H), 3.16-3.09 (m, 1H), 3.01-2.93 (m, 1H), 2.72-2.66 (m, 1H), 2.54-2.48 (m, 4H), 2.07-1.91 (m, 3H), 1.72-1.65 (m, 1H), 1.28 (s, 3H), 1.27 (s, 3H).

### Preparation of Intermediate S44

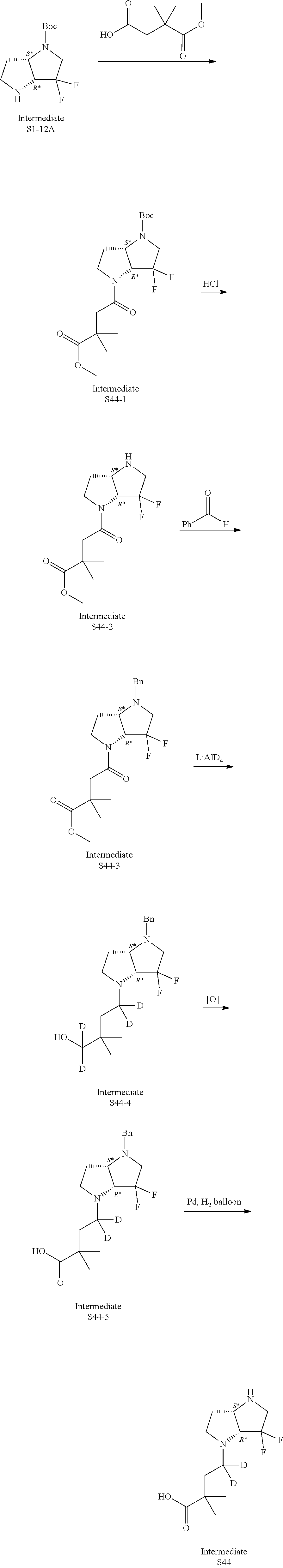

### S44-1: (cis)-tert-Butyl 3,3-difluoro-4-(4-methoxy-3,3-dimethyl-4-oxobutanoyl)hexahydropyrrolo[3,2-b]pyrrole-1(2H)-carboxylate

To a solution of S1-12A (2.00 g, 95% purity, 8.06 mmol) and 4-methoxy-3,3-dimethyl-4-oxobutanoic acid (1.68 g, 85% purity, 10.5 mmol) in dichloromethane (30 mL) was added N,N-diisopropylethylamine (3.20 g, 24.2 mmoL) and o-(7-azabenzotriazol-1-yl)-N,N,N,N-tetramethyluronium hexafluorophosphate (6.20 g, 16.1 mmol). After stirred at room temperature overnight under nitrogen atmosphere, the reaction mixture was added into ethyl acetate (50 mL). The organic layer was washed with water (20 mL) and brine (20 mL), dried over Na₂SO₄₍ₛ₎, filtered and concentrated to give a residue, which was purified by silica gel column chromatography (petroleum ether:ethyl acetate=8:1) to get the desired product (3.00 g, 100% purity, 81% yield) as yellow oil. LC-MS (ESI): mass calcd. for C₁₈H₂₈F₂N₂O₅ 390.2, m/z found 391.4 [M+H]⁺.

### S44-2: Methyl 4-((cis)-4-Benzyl-6,6-difluorohexahydropyrrolo[3,2-b]pyrrol-1(2H)-yl)-2,2-dimethyl-4-oxobutanoate

To a solution of S44-1 (1.00 g, 100% purity, 2.56 mmol) in ethyl acetate (0.5 mL) was added 4 M hydrochloride in ethyl acetate (20 mL). After stirred at room temperature overnight, the mixture was concentrated and the residue was poured into ethyl acetate (30 mL). The solution was washed with saturated sodium bicarbonate aqueous solution (20 mL), water (20 mL) and brine (20 mL), dried over Na₂SO₄₍ₛ₎ and filtered. The filtrate was concentrated to give the desired compound (570 mg, 100% purity, 77% yield) as yellow oil. LC-MS (ESI): R_{T}=1.28 min, mass calcd. for C₁₃H₂₀F₂N₂O₃ 290.1, m/z found 291.4 [M+H]⁺.

### S44-3: Methyl 4-((cis)-4-benzyl-6,6-difluorohexahydropyrrolo[3,2-b]pyrrol-1(2H)-yl)-2,2-dimethyl-4-oxobutanoate

To a mixture of S44-2 (570 mg, 100% purity, 1.96 mmol) in dichloromethane (15 mL) was added benzaldehyde (225 mg, 2.40 mmol), acetic acid (1 mL, 17.5 mmol) and 1 M titanium (IV) triisopropoxy chloride in tetrahydrofuran (4 mL, 4 mmol). After stirred for 1 hour at room temperature, sodium triacetoxyborohydride (2.00 g, 9.80 mmoL) was added. After stirred at room temperature overnight, the reaction mixture was quenched by saturated sodium bicarbonate aqueous solution (30 mL) and dichloromethane (30 mL). The organic layer was separated and washed with brine (20 mL), dried over Na₂SO₄₍ₛ₎ and filtered. The filtrate was concentrated and purified by C18 column (acetonitrile:water (+0.2% ammonium bicarbonate)=20% to 70%) to afford the desired product (721 mg, 100% purity, 96% yield) as yellow oil. LC-MS (ESI): mass calcd. for C₂₀H₂₆F₂N₂O₃ 380.2, m/z found 381.5 [M+H]⁺.

### S44-4: 4-((cis)-4-Benzyl-6,6-difluorohexahydropyrrolo[3,2-b]pyrrol-1(2H)-yl)-1,1,4,4-tetradeutero-2,2-dimethylbutan-1-ol

To a mixture of S44-3 (721 mg, 100% purity, 1.89 mmol) in tetrahydrofuran (10 mL) was added lithium aluminum deuteride (880 mg, 21.0 mmol). After stirred at 60° C. overnight under nitrogen atmosphere, the reaction mixture was cooled down to room temperature and quenched with Na₂SO₄.10H₂O. The mixture was filtered and the filtrate was concentrated to afford the desired product (612 mg, 93% purity, 84% yield) as yellow oil. LC-MS (ESI): mass calcd. for C₁₉H₂₄D₄F₂N₂O342.2, m/z found 343.5 [M+H]⁺.

### S44-5: 4-((cis)-4-Benzyl-6,6-difluorohexahydropyrrolo[3,2-b]pyrrol-1(2H)-yl)-4,4-dideutero-2,2-dimethylbutanoic acid

To a mixture of chromium(VI) oxide (210 mg, 2.10 mmol) in water (0.3 mL) was added sulfuric acid (0.15 mL) dropwise. Then water (0.6 mL) was added dropwise. The solution was stirred at room temperature for 0.5 hour. The solution was added to a solution of S44-4 (612 mg, 93% purity, 1.60 mmol) in acetone (6 mL) at 0° C. dropwise. After stirred at room temperature for 2 hours, the mixture was basified with saturated sodium carbonate aqueous solution to pH=6. The mixture was extracted with ethyl acetate (30 mL) for five times. The combined organic phases were washed with brine (10 mL) twice, dried over Na₂SO₄₍ₛ₎, filtered and concentrated. The residue was purified by C18 column (acetonitrile:water=10% to 70%) to afford the desired product (150 mg, 52% purity, 12% yield) as yellow oil. LC-MS (ESI): mass calcd. for C₁₉H₂₄D₂F₂N₂O₂ 354.2, m/z found 355.5 [M+H]⁺.

### S44: 4,4-Dideutero-4-((cis)-6,6-difluorohexahydropyrrolo[3,2-b]pyrrol-1(2H)-yl)-2,2-dimethylbutanoic acid

To a solution of S44-5 (150 mg, 52% purity, 0.22 mmol) in isopropanol (10 mL) was added palladium (II) acetate (50 mg, 0.60 mmol) and active carbon (50 mg). The reaction mixture was stirred at 50° C. for 6 hours under hydrogen atmosphere (balloon). The reaction mixture was filtered and the filtrate was concentrated to afford the desired product (150 mg, 32% purity, 82% yield) as yellow oil. LC-MS (ESI): mass calcd. for C₁₂H₁₈D₂F₂N₂O₂ 264.2, m/z found 265.4 [M+H]⁺.

### Compound 137: 4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylbutanoic-4,4-d2 acid

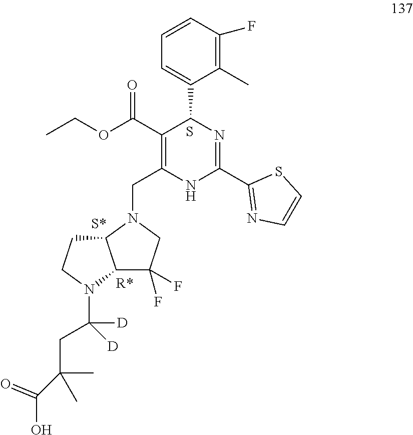

This compound is made from H2-1A and S44 according to typical method 1. Purified by Prep. HPLC (Column: waters xbrige C18 (5 μm 19*150 mm), Mobile phase A: water (+0.1% ammonium bicarbonate), Mobile phase B: acetonitrile, UV: 254 nm, Flow rate: 15 mL/min, Gradient: 05-95% (% B)) to afford the desired product (43.7 mg, 95% purity by ¹H NMR, 38% yield) as yellow solids. LC-MS (ESI): mass calcd. for C₃₀H₃₄D₂F₃N₅O₄S 621.3, m/z found 622.3 [M+H]⁺. ¹H NMR (400 MHz, CDCl₃) δ 9.24 (s, 1H), 7.84 (d, J=3.2 Hz, 1H), 7.41 (d, J=3.2 Hz, 1H), 7.10-7.04 (m, 1H), 6.98-6.96 (m, 1H), 6.92-6.88 (m, 1H), 6.00 (s, 1H), 4.29-4.24 (m, 1H), 4.12-4.02 (m, 3H), 3.86-3.81 (m, 1H), 3.43-3.32 (m, 3H), 3.00-2.92 (m, 1H), 2.54 (s, 3H), 2.50-2.48 (m, 1H), 2.04-1.92 (m, 3H), 1.67-1.64 (m, 1H), 1.28 (s, 3H), 1.27 (s, 3H), 1.10 (t, J=7.2 Hz, 3H).

### Compound 138: 4-((cis)-4-((6-(2-chloro-3,4-difluorophenyl)-5-(ethoxycarbonyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

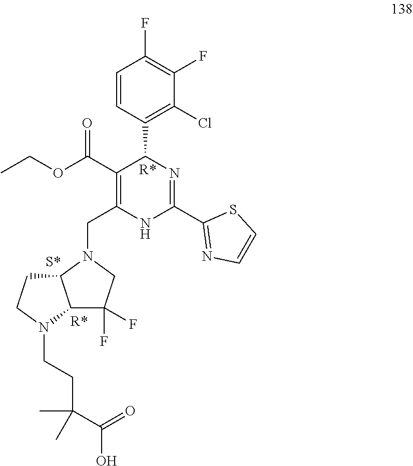

This compound was made from H8-1A and S9 according to typical coupling method 1 and typical method 3 successively. LC-MS (ESI): mass calcd. for C₂₉H₃₂ClF₄N₅O₄S 657.2, m/z found 658.2 [M+H]⁺. ¹H NMR (400 MHz, DMSO-d6) δ 12.11 (br s, 1H), 9.49 (s, 1H), 7.94-8.03 (m, 2H), 7.39-7.49 (m, 1H), 7.27 (m, 1H), 6.04 (s, 1H), 4.12 (s, 2H), 3.83-4.02 (m, 3H), 3.06-3.14 (m, 2H), 3.16-3.33 (m, 2H), 2.55-2.76 (m, 1H), 2.39-2.49 (m, 1H), 2.18-2.39 (m, 1H), 1.70-1.89 (m, 2H), 1.62-1.68 (m, 2H), 1.08-1.13 (m, 6H), 1.01-1.04 (t, J=7.2 Hz, 3H).

### Compound 139: 4-((cis)-4-((6-(3,4-difluoro-2-methylphenyl)-5-(ethoxycarbonyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

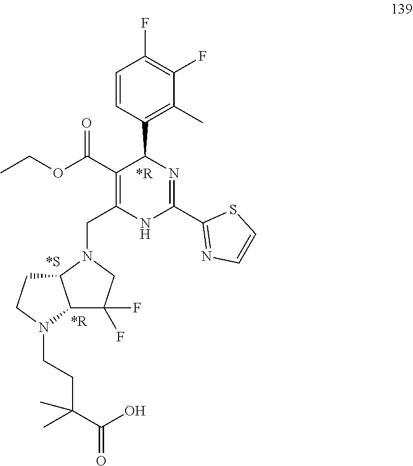

This compound was made from H9-1A and S9 analogous to compound 7A. LCMS (ESI): mass calcd. for C₃₀H₃₅F₄N₅O₄S 637.7, m/z found 638.3 [M+H]⁺. ¹H NMR (400 MHz, METHANOL-d₄) δ 7.94-7.87 (m, 1H), 7.77-7.66 (m, 1H), 7.11-6.96 (m, 2H), 5.94-5.86 (m, 1H), 4.77-4.57 (m, 2H), 4.31-4.10 (m, 2H), 4.10-3.99 (m, 2H), 3.94-3.83 (m, 1H), 3.40-3.34 (m, 1H), 3.14-2.98 (m, 1H), 2.93-2.79 (m, 1H), 2.71-2.45 (m, 6H), 2.09-1.86 (m, 2H), 1.86-1.75 (m, 2H), 1.24-1.17 (m, 6H), 1.16-1.06 (m, 3H). ¹⁹F NMR (METHANOL-d₄, 400 MHz): δ −101.9, −117.1, −141.8, −143.7.

### Compound 140: (cis)-4-(4-((5-(ethoxycarbonyl)-2-(thiazol-2-yl)-6-(2,3,4-trifluorophenyl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

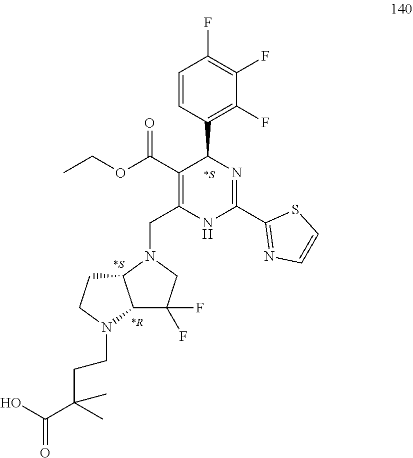

This compound was made from H25-1A and S9 analogous to compound 7A. LCMS (ESI): mass calcd. for C₂₉H₃₂F₅N₅O₄S 641.2, m/z found 642.3 [M+H]⁺. ¹H NMR (400 MHz, METHANOL-d4) δ ppm 7.97 (1H, d, J=3.18 Hz), 7.85 (1H, d, J=3.18 Hz), 7.18-7.25 (1H, m), 7.07-7.14 (1H, m), 6.01 (1H, s), 4.40-4.48 (1H, m), 4.04-4.22 (5H, m), 3.92 (1H, dt, J=11.46, 6.74 Hz), 3.60-3.70 (1H, m), 3.40-3.52 (1H, m), 3.32-3.40 (2H, m), 3.20-3.29 (1H, m), 2.30-2.46 (2H, m), 1.97 (2H, dt, J=11.22, 5.70 Hz), 1.27 (6H, s), 1.16 (3H, t, J=7.09 Hz).

### Compound 141: 4-((cis)-4-(((R)-6-(2-chloro-4-fluorophenyl)-5-(ethoxycarbonyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

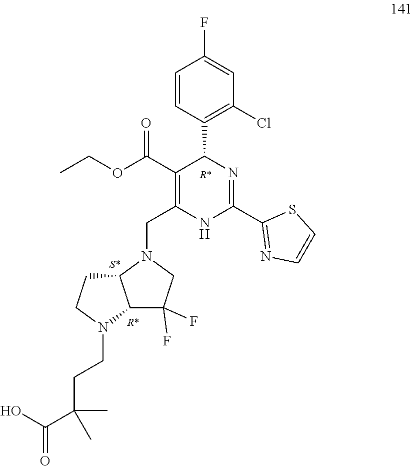

This compound was made from H12-1A and S9 analogous to compound 7A. Purified by C18 column (MeCN:water containing 0.5% TFA=10% to 60%) to give title product (135.9 mg, yield 89.617%) as a yellow solid. LCMS (ESI): mass calcd. for C₂₉H₃₃ClF₃N₅O₄S 639.19, m/z found 640.3 [M+H]⁺. ¹H NMR (400 MHz, CD₃Cl) δ=7.89 (d, J=4.0 Hz, 1H), 7.63 (d, J=4.0 Hz, 1H), 7.43 (m, 1H), 7.15 (dd, J₁=4.0, J₂=8.0 Hz, 1H), 7.04-6.98 (m, 1H), 6.19 (s, 1H), 4.62 (m, 1H), 4.51 (m, 1H), 4.34 (m, 1H), 4.20 (t, J=8.0 Hz, 1H), 4.05 (q, J=8.0 Hz, 2H), 3.82 (m, 1H), 3.69 (m, 1H), 3.44-3.31 (m, 3H), 2.79-2.74 (m, 1H), 2.69-2.62 (m, 1H), 2.35-2.24 (m, 1H), 2.22-2.11 (m, 1H), 1.67-1.59 (m, 1H), 1.31-1.19 (m, 6H), 1.11 (t, J=8.0 Hz, 3H).

### Compound 142: (cis)-4-(4-(((S)-6-(2,3-difluorophenyl)-5-(ethoxycarbonyl)-2-(thiazol-2-yl)-3,6-dihydropyrimidin-4-yl)methyl)-6,6-difluorohexahydropyrrolo[3,2-b]pyrrol-1(2H-yl)-2,2-dimethylbutanoic acid (Single Diastereomer)

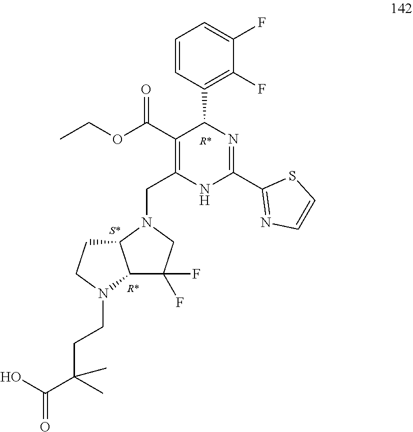

This compound was made from H24-1A and S9 analogous to compound 7A. LCMS (ESI): mass calcd. for C₂₉H₃₃F₄N₅O₄S 623.2, found m/z 624.3 [M+H]⁺. ¹H NMR (400 MHz, METHANOL-d4) δ ppm 7.99 (1H, d, J=3.18 Hz), 7.88 (1H, d, J=3.06 Hz), 7.11-7.25 (3H, m), 6.06 (1H, s), 4.39-4.48 (1H, m), 4.04-4.19 (5H, m), 3.92 (1H, dt, J=11.43, 6.76 Hz), 3.66 (1H, td, J=12.07, 4.10 Hz), 3.32-3.50 (3H, m), 3.20-3.28 (1H, m), 2.31-2.46 (2H, m), 1.91-2.04 (2H, m), 1.26 (6H, s), 1.16 (3H, t, J=7.09 Hz).

### Compound 143A and 143B: 3-(((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)methyl)cyclobutane-1-carboxylic acid

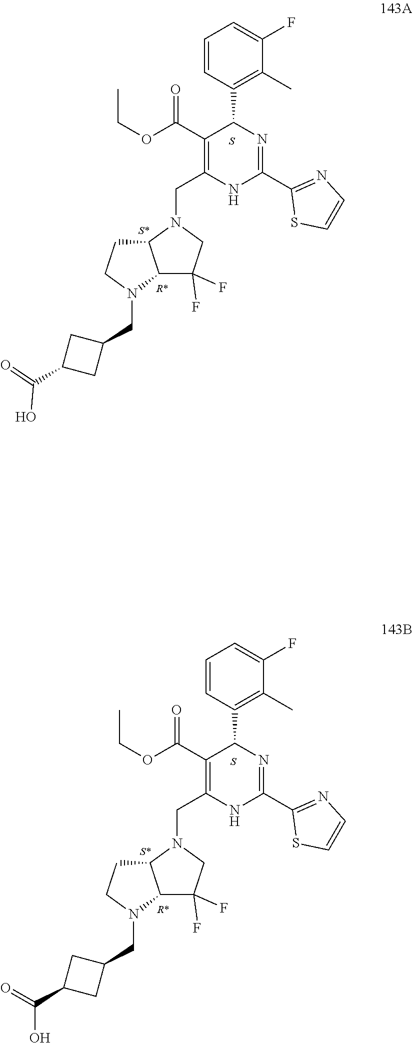

These two compound were made from Compound 103 and ethyl 3-formylcyclobutane-1-carboxylate using typical method 5 and typical method 4 successively.

143A: yellow solid. LC-MS (ESI): mass calcd. for C₃₀H₃₄F₃N₅O₄S 617.23, m/z found 618.3 [M+H]⁺. ¹H NMR (400 MHz, CD₃OD) δ 7.95 (d, J=4.0 Hz, 1H), 7.84 (d, J=4.0 Hz, 1H), 7.21-7.14 (m, 2H), 6.98 (m, 1H), 6.00 (s, 1H), 4.37-4.27 (m, 1H), 4.22-4.20 (m, 2H), 4.15-4.11 (m, 1H), 4.06 (q, J=7.1 Hz, 2H), 3.90-3.80 (m, 1H), 3.74-3.62 (m, 1H), 3.48-3.36 (m, 4H), 3.14-3.08 (m, 1H), 2.94-2.82 (m, 1H), 2.53-2.39 (m, 6H), 2.24-2.21 (m, 1H), 2.29-2.16 (m, 2H), 1.10 (t, J=7.1 Hz, 3H).

143B: yellow solid. LC-MS (ESI): mass calcd. for C₃₀H₃₄F₃N₅O₄S 617.23, m/z found 618.3 [M+H]⁺. ¹H NMR (400 MHz, CD₃OD) 7.97 (d, J=4.0 Hz, 1H), 7.86 (d, J=4.0 Hz, 1H), 7.21-7.14 (m, 2H), 6.98 (m, 1H), 6.01 (s, 1H), 4.38-4.30 (m, 1H), 4.27-4.13 (m, 2H), 4.06 (m, 3H), 3.90-3.82 (m, 1H), 3.72-3.63 (m, 1H), 3.42-3.33 (m, 3H), 3.29-3.23 (m, 1H), 3.17-3.07 (m, 1H), 2.77-2.62 (m, 1H), 2.52-2.41 (m, 6H), 2.38-2.28 (m, 1H), 2.19-2.03 (m, 2H), 1.13-1.08 (m, J=8.0 Hz, 3H).

### Compound 144: ethyl (S)-6-(((cis)-4-(5-(tert-butoxy)-4,4-dimethyl-5-oxopentyl)-3,3-difluorohexahydropyrrolo[3,2-b]pyrrol-1(2H)-yl)methyl)-4-(3-fluoro-2-methylphenyl)-2-(thiazol-2-yl)-1,4-dihydropyrimidine-5-carboxylate (Single Diastereomer)

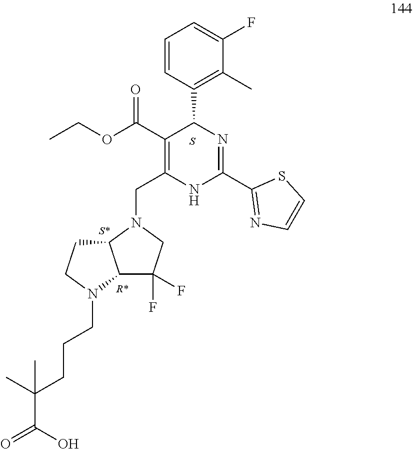

This compound was made from Compound 103 and tert-butyl 2,2-dimethyl-5-oxopentanoate according to typical method 5 and typical method 3, successively. LCMS (ESI): mass calcd. for C₃₁H₃₈F₃N₅O₄S 633.2, m/z found 634.3 [M+H]⁺. ¹H NMR (400 MHz, DMSO-d₆) δ 8.00 (brs, 2H), 7.19 (m, 1H), 7.14 (m, 1H), 7.06 (m, 1H), 5.86 (s, 1H), 4.42-3.65 (m, 8H), 3.34 (m, 4H), 3.09 (m, 2H), 2.43 (s, 3H), 2.16 (m, 2H), 1.58 (m, 2H), 1.48 (m, 2H), 1.11 (s, 6H), 1.05 (t, J=7.2 Hz, 3H).

### Compound 145: 4-((cis)-4-((6-(2-chloro-3-fluorophenyl)-5-(ethoxycarbonyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

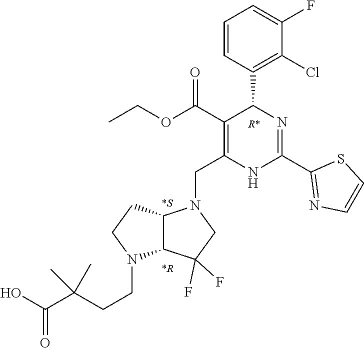

This compound was made from H1-1A and S9 according to typical method 1 and 3. purified by C18 (acetonitrile:water=05% to 60%) to give desired compound (86.9 mg, 99.6% purity, 67% yield) as yellow solids. LC-MS (ESI): mass calcd. for C₂₉H₃₃ClF₃N₅O₄S 639.2, m/z found 519.9 [M+H]⁺. ¹H NMR (400 MHz, CDCl₃) δ 9.31 (s, 1H), 7.86 (d, J=2.8 Hz, 1H), 7.43 (d, J=3.2 Hz, 1H), 7.20-7.11 (m, 2H), 7.06-7.02 (m, 1H), 6.26 (s, 1H), 4.24 (d, J=17.6 Hz, 1H), 4.09-3.99 (m, 3H), 3.85-3.80 (m, 1H), 3.45-3.31 (m, 3H), 3.15-3.07 (m, 1H), 3.00-2.92 (m, 1H), 2.70-2.65 (m, 1H), 2.53-2.47 (m, 1H), 2.06-1.91 (m, 3H), 1.71-1.65 (m, 1H), 1.28 (s, 3H), 1.27 (s, 3H), 1.10 (t, J=7.2 Hz, 3H).

### Compound 146: 4-((cis)-4-((5-(Ethoxycarbonyl)-6-(3-fluoro-2-methylphenyl)-2-(1-methyl-1H-imidazol-2-yl)-3,6-dihydropyrimidin-4-yl)methyl)-6,6-difluorohexahydropyrrolo[3,2-b]pyrrol-1(2H)-yl)-2,2-dimethylbutanoic acid (Single Diastereomer)

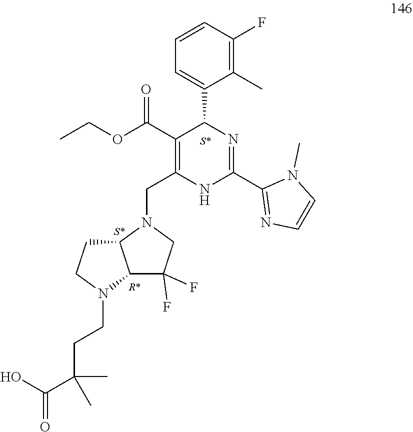

This compound was made from H28-1B and S9 according to typical method 1 and 3. LC-MS (ESI): mass calcd. for C₃₁H₃₉F₃N₆O₄ 616.3, m/z found 617.3 [M+H]⁺. ¹H NMR (400 MHz, CD₃OD) δ 7.19-7.13 (m, 2H), 7.05 (d, J=7.2 Hz, 1H), 7.01 (s, 1H), 6.96-6.92 (m, 1H), 6.01 (s, 1H), 4.19 (s, 2H), 4.06 (q, J=7.2 Hz, 2H), 2.94-3.90 (m, 1H), 3.87 (s, 3H), 3.43-3.37 (m, 1H), 3.28-3.23 (m, 2H), 3.09-2.99 (m, 1H), 2.90-2.82 (m, 1H), 2.59-2.57 (m, 1H), 2.54 (s, 3H), 2.49-2.41 (m, 1H), 2.06-1.90 (m, 2H), 1.82-1.78 (m, 2H), 1.20 (s, 6H), 1.14 (t, J=7.2 Hz, 3H).

### Compound 147: 4-((cis)-4-(((S)-5-(Ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)-2-methylbutanoic acid

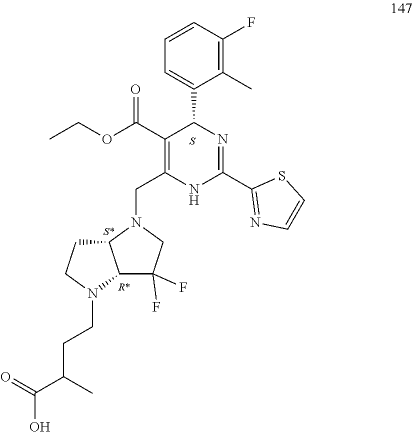

This compound was made from compound 103 and tert-Butyl 2-methyl-4-oxobutanoate According to typical method 5 and 3 successively. LC-MS (ESI): mass calcd. for C₂₉H₃₄F₃N₅O₄S 605.2, m/z found 605.9 [M+H]⁺. ¹H NMR (400 MHz, CDCl₃) δ 9.25-9.20 (d, J=18.4 Hz, 1H), 7.85-7.82 (m, 1H), 7.40-7.39 (d, J=3.2 Hz, 1H), 7.09-7.04 (m, 1H) 6.98-6.96 (d, J=6.8 Hz, 1H), 6.92-6.88 (m, 1H), 6.00 (s, 1H), 4.27-4.22 (m, 1H), 4.12-4.01 (m, 3H), 3.87-3.81 (m, 1H), 3.50-3.27 (m, 3H), 3.17-2.93 (m, 2H), 2.75-2.71 (m, 1H), 2.61-2.57 (m, 1H), 2.53 (s, 3H), 2.39-2.37 (m, 1H), 2.06-1.78 (m, 4H), 1.29-1.23 (m, 3H), 1.12-1.08 (m, 3H).

### Preparation of Intermediate S56

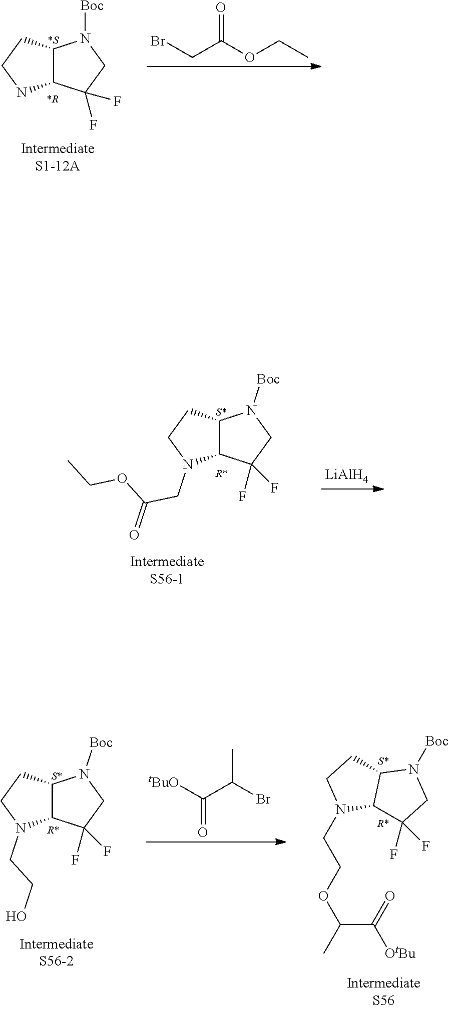

### S56-1: (cis)-tert-Butyl 4-(2-ethoxy-2-oxoethyl)-3,3-difluorohexahydropyrrolo[3,2-b]pyrrole-1(2H)-carboxylate

To a solution of S1-12A (1.5 g, 90% purity, 5.44 mmol) in N,N-dimethylformamide (10 mL) was added ethyl 2-bromoacetate (1.1 g, 6.59 mmol) and potassium carbonate (1.5 g, 10.9 mmol) at room temperature. After stirred at room temperature overnight, the mixture was extracted with ethyl acetate (30 mL) twice. The combined organic layers were washed with water (20 mL) and brine (20 mL), dried over Na₂SO₄₍ₛ₎ and filtered. The filtrate was concentrated to give a residue, which was purified by silica gel column chromatography (petroleum ether:ethyl acetate=20:1 to 5:1) to give the title compound (1.43 g, 90% purity from ¹H NMR, 71% yield) as colorless oil. ¹H NMR (400 MHz, CDCl₃) δ 4.56-4.44 (m, 1H), 4.17 (q, J=7.2 Hz, 2H), 3.91-3.74 (m, 1H), 3.66-3.49 (m, 3H), 3.28-3.25 (m, 1H), 2.84-2.74 (m, 1H), 2.34-2.26 (m, 1H), 2.03-1.92 (m, 2H), 1.47 (s, 4.5H), 1.46 (s, 4.5H), 1.28 (t, J=7.2 Hz, 3H).

### S56-2: (cis)-tert-Butyl 3,3-difluoro-4-(2-hydroxyethyl)hexahydropyrrolo[3,2-b]pyrrole-1(2H)-carboxylate

To a solution of S56-1 (1.43 g, 90% purity, 3.85 mmol) in dichloromethane (20 mL) was slowly added lithium aluminum hydride (250 mg, 6.59 mmol) at 0° C. After stirred at 0° C. for 2 hours, the mixture was quenched with water (20 mL) at 0° C. and then stirred for 10 minutes. The mixture was filtered and filtrate was concentrated under reduced pressure to give the title compound (700 mg, 90% purity from ¹H NMR, 56% yield) as white oil. ¹H NMR (400 MHz, CDCl₃) δ 4.54-4.34 (m, 1H), 3.92-3.79 (m, 1H), 3.71-3.60 (m, 3H), 3.29-3.21 (m, 2H), 3.14-3.08 (m, 1H), 2.63-2.61 (m, 1H), 2.44-2.40 (m, 1H), 1.88-1.76 (m, 2H), 1.46 (s, 9H).

### S56: (cis)-tert-Butyl 4-(2-((1-(tert-butoxy)-1-oxopropan-2-yl)oxy)ethyl)-3,3-difluorohexahydropyrrolo[3,2-b]pyrrole-1(2H)-carboxylate

To a solution of S56-2 (100 mg, 90% purity, 0.308 mmol) in toluene (4 mL) was added tert-butyl 2-bromopropanoate (320 mg, 1.53 mmol) and tetrabutylammonium bromide (26 mg, 0.077 mmol), followed by an aqueous solution of sodium hydroxide (0.8 mL, 0.5 g/mL). After stirred at room temperature overnight, the mixture was diluted with water (20 mL) and extracted with ethyl acetate (20 mL) for three times. The combined organic layers were washed with brine (20 mL), dried over Na₂SO₄₍ₛ₎, filtered and concentrated to give a residue, which was purified by silica gel column chromatography (petroleum ether:ethyl acetate=5:1) to give the title compound (83 mg, 90% purity from ¹H NMR, 58% yield) as yellow oil. ¹H NMR (400 MHz, CDCl₃) δ 4.48-4.39 (m, 1H), 3.91-3.49 (m, 5H), 3.29-3.10 (m, 3H), 2.71-2.67 (m, 1H), 2.48-2.43 (m, 1H), 2.31-2.22 (m, 1H), 1.85 (br s, 1H), 1.47 (s, 9H), 1.46 (s, 9H), 1.36-1.34 (m, 3H).

### Compound 148: 2-(2-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)ethoxy)propanoic acid

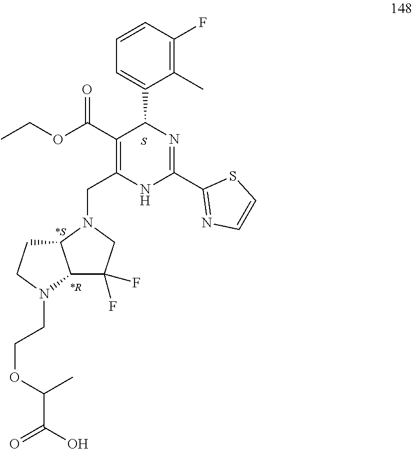

This compound was made from S56 and H2-1A according to typical method 1 and 3. LC-MS (ESI): mass calcd. for C₂₉H₃₄F₃N₅O₅S 621.2, m/z found 622.3. ¹H NMR (400 MHz, CD₃OD) δ 7.92 (dd, J=3.2, 1.6 Hz, 1H), 7.73 (d, J=3.2 Hz, 1H), 7.19-7.11 (m, 2H), 6.97-6.92 (m, 1H), 5.98 (s, 1H), 4.27 (d, J=16.8 Hz, 1H), 4.15 (d, J=16.8 Hz, 1H), 4.10-4.02 (m, 3H), 3.99-3.92 (m, 1H), 3.79-3.73 (m, 1H), 3.69-3.62 (m, 2H), 3.43-3.36 (m, 3H), 3.15-3.06 (m, 1H), 2.99-2.95 (m, 1H), 2.83-2.76 (m, 1H), 2.52 (d, J=2.0 Hz, 3H), 2.08-2.00 (m, 2H), 1.40 (d, J=6.0 Hz, 3H), 1.14 (t, J=7.2 Hz, 3H).

### Compound 149A and 149B: 3-((cis)-4-(((R)-6-(2-chloro-3-fluorophenyl)-5-(ethoxycarbonyl)-2-(thiazol-2-yl)-3,6-dihydropyrimidin-4-yl)methyl)-6,6-difluorohexahydropyrrolo[3,2-b]pyrrol-1(2H)-yl)-2-methylpropanoic acid (Single Diastereomers)

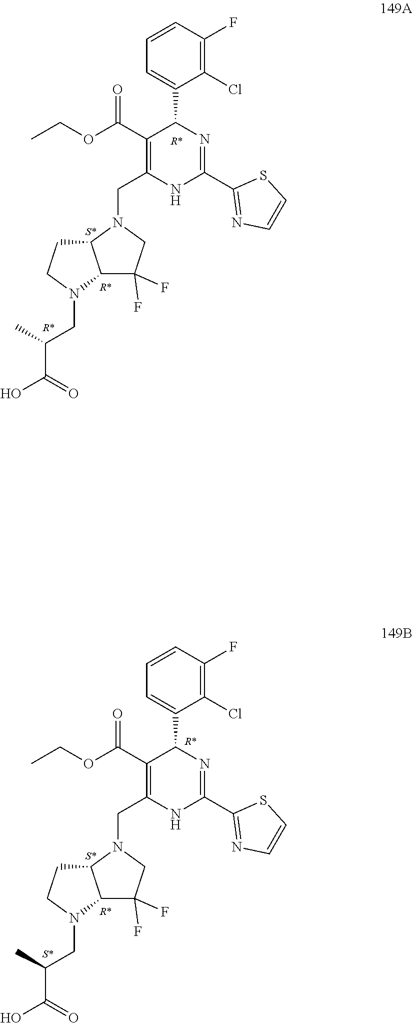

149A was made from H1-1A and S43A according to typical method 1 and typical method 3 successively. LC-MS (ESI): mass calcd. for C₂₇H₂₉ClF₃N₅O₄S 611.2, m/z found 612.2 [M+H]⁺. ¹H NMR (400 MHz, CDCl₃) δ 9.28 (s, 1H), 7.86 (d J=3.2 Hz, 1H), 7.44 (d, J=3.2 Hz, 1H), 7.19-7.11 (m, 2H), 7.07-7.02 (m, 1H), 6.27 (s, 1H), 4.33 (s, 1H), 4.05-3.90 (m, 3H), 3.86-3.81 (m, 1H), 3.70-3.62 (m, 1H), 3.36-3.25 (m, 2H), 3.07-2.89 (m, 4H), 2.86-2.58 (m, 1H), 2.03-21.95 (m, 2H), 1.21 (d, J=7.2 Hz, 3H), 1.10 (t, J=7.2 Hz, 3H).

149B was made from H1-1A and S43B: LC-MS (ESI): mass calcd. for C₂₇H₂₉ClF₃N₅O₄S 611.2, m/z found 612.2 [M+H]⁺. ¹H NMR (400 MHz, CDCl₃) δ 9.33 (1H, s), 7.90 (d, J=3.2 Hz, 1H), 7.44 (d, J=3.2 Hz, 1H), 7.19-7.10 (m, 2H), 7.07-6.99 (m, 1H), 6.27 (s, 1H), 4.28 (s, 1H), 4.09-3.97 (m, 3H), 3.91-3.84 (m, 1H), 3.60-3.52 (m, 1H), 3.47-3.35 (m, 2H), 3.10-2.97 (m, 2H), 2.80-2.74 (m, 1H), 2.69-2.57 (m, 2H), 2.19-2.10 (m, 1H), 2.07-1.98 (m, 1H), 1.23 (d, J=7.2 Hz, 3H), 1.10 (t, J=7.2 Hz, 3H).

### Preparation of Intermediate S58

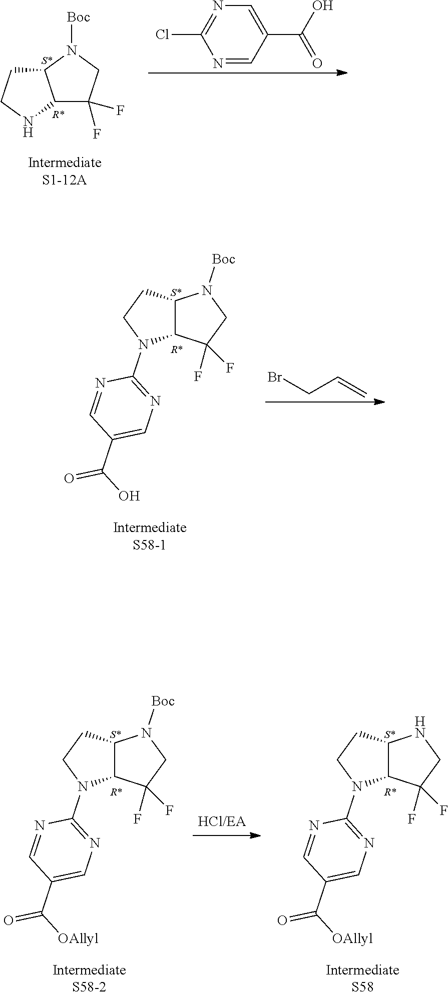

### S58-1: 2-((cis)-4-(tert-Butoxycarbonyl)-6,6-difluorohexahydropyrrolo[3,2-b]pyrrol-1(2H)-yl)pyrimidine-5-carboxylic acid

To a solution of S1-12A (200 mg, 95% purity, 0.8 mmol) in 1,4-dioxane (3 mL) was added 2-chloropyrimidine-5-carboxylic acid (190 mg, 1.20 mmol) and N,N-diisopropylethylamine (260 mg, 2.0 mmol) at room temperature. After stirred at 100° C. for 2 hours, the mixture was concentrated to give the crude product, which was purified by C18 column (acetonitrile:water=5% to 95%) to give the title compound (150 mg, 94% purity, 50% yield) as white solids. LC-MS (ESI): mass calcd. for C₁₆H₂₀F₂N₄O₄ 370.2, m/z found 371.4 [M+H]⁺.

### S58-2: (cis)-tert-Butyl 4-(5-((allyloxy)carbonyl)pyrimidin-2-yl)-3,3-difluorohexahydropyrrolo[3,2-b]pyrrole-1(2H)-carboxylate

To a solution of S58-1 (150 mg, 94% purity, 0.41 mmol) in N,N-dimethylformamide (3 mL) was added 4-bromobut-1-ene (74 mg, 0.615 mmol) and potassium carbonate (114 mg, 0.82 mmol) at room temperature. After stirred at room temperature for 16 hours, the mixture was diluted with water (30 mL) and extracted with ethyl acetate (30 mL) for three times. The combine organic layers were washed with brine (150 mL), dried over Na₂SO₄₍ₛ₎ and concentrated to give the crude product (80 mg, 47% yield) as yellow oil. LC-MS (ESI): mass calcd. for C₁₉H₂₄F₂N₄O₄ 410.2, m/z found 411.5 [M+H]⁺.

### S58: Allyl 2-((cis)-6,6-difluorohexahydropyrrolo[3,2-b]pyrrol-1(2H)-yl)pyrimidine-5-carboxylate hydrochloride

To a solution of S58-2 (80 mg, 0.2 mmol) in ethyl acetate was added 4 M hydrochloride in ethyl acetate (10 mL) at room temperature. After stirred at room temperature for 2 hours, the mixture was concentrated to give the title compound (68 mg, 61% purity, 45% yield) as yellow solids. LC-MS (ESI): mass calcd. for C₁₄H₁₇ClF₂N₄O₂ 346.1, m/z found 311.4 [M−HCl+H]⁺.

### Compound 150: 2-((cis)-4-(((S)-5-(Ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)pyrimidine-5-carboxylic acid

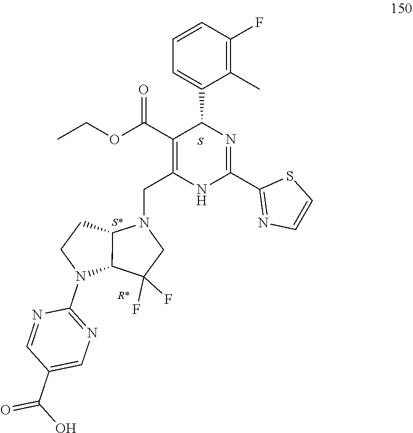

This compound was made from S58 and H2-1A according to typical method 1 and 2 successively. LC-MS (ESI): mass calcd. for C₂₉H₂₈F₃N₇O₄S 627.2, m/z found 628.1 [M+H]⁺. ¹H NMR (400 MHz, CD₃OD) δ 9.25 (s, 1H), 8.98 (s, 2H), 7.78 (d, J=3.2 Hz, 1H), 7.38 (d, J=2.8 Hz, 1H), 711-7.06 (m, 1H), 7.02-7.00 (m, 1H), 6.91-6.89 (m, 1H), 6.03 (s, 1H), 5.04-4.96 (m, 1H), 4.42-4.34 (m, 2H), 4.09-4.05 (m, 3H), 3.91-3.80 (m, 2H), 3.40-3.34 (m, 1H), 3.00-2.91 (m, 1H), 2.54 (s, 3H), 2.23-1.95 (m, 2H), 1.11 (t, J=7.2 Hz, 3H).

### Compound 151: 4-((cis)-6,6-difluoro-4-(((S)-6-(3-fluoro-2-methylphenyl)-5-(propoxycarbonyl)-2-(thiazol-2-yl)-3,6-dihydropyrimidin-4-yl)methyl)hexahydropyrrolo[3,2-b]pyrrol-1(2H)-yl)-2,2-dimethylbutanoic acid

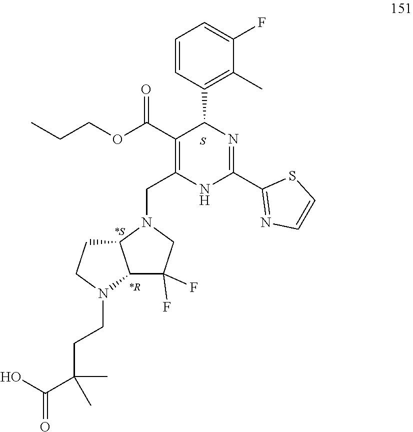

This compound was made analogous to compound 152 from S73-2, 1-iodopropane and S9. LC-MS (ESI): mass calcd. for C₃₁H₃₈F₃N₅O₄S 633.3, m/z found 634.3 [M+H]⁺. ¹H NMR (400 MHz, CDCl₃) δ 7.84 (d, J=3.2 Hz, 1H), 7.41 (d, J=3.2 Hz, 1H), 7.10-7.05 (m, 1H), 6.97 (d, J=7.6 Hz, 1H), 6.92-6.88 (m, 1H), 6.01 (s, 1H), 4.70 (s, 1H), 4.28 (d, J=17.6 Hz, 1H), 4.11 (d, J=17.2 Hz, 1H), 3.94 (t, J=6.8 Hz, 2H), 3.85-3.80 (m, 1H), 3.45-3.28 (m, 3H), 3.13-3.06 (m, 1H), 3.00-2.92 (m, 1H), 2.68-2.62 (m, 1H), 2.54 (d, J=2.0 Hz, 3H), 2.50-2.44 (m, 1H), 2.04-1.92 (m, 3H), 1.70-1.64 (m, 1H), 1.54-1.48 (m, 2H), 1.28 (s, 3H), 1.27 (s, 3H), 0.74 (t, J=7.6 Hz, 3H).

### Preparation of Intermediate S73

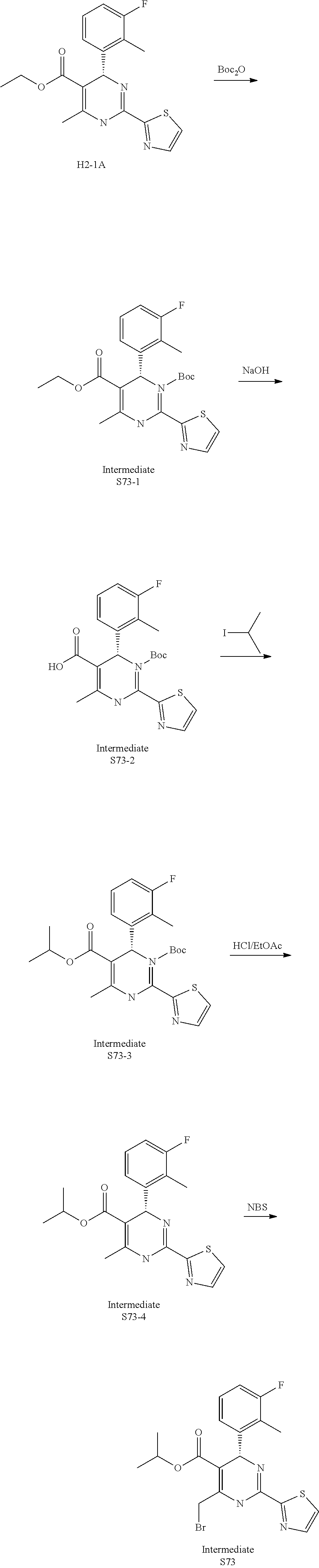

### S73-1: (S)-ethyl6-(3-fluoro-2-methylphenyl)-4-methyl-2-(thiazol-2-yl)-1,6-dihydropyrimidine-5-carboxylate

To a solution of (S)-ethyl 4-(3-fluoro-2-methylphenyl)-6-methyl-2-(thiazol-2-yl)-1,4-dihydropyrimidine-5-carboxylate H2-1A (2 g, 95% purity, 5.33 mmol) and di-tert-butyl dicarbonate (1.5 g, 6.87 mmol) in dichloromethane (20 mL) was added triethylamine (1.0 g, 9.88 mmol) and N,N-dimethylpyridin-4-amine (100 mg, 0.819 mmol). After stirred at room temperature for 6 hours, the mixture was concentrated and purified by silica gel column chromatography (petroleum ether:ethyl acetate=15;1 to 10:1) to give the title compound (2.5 g, 95% purity from ¹H NMR, 98% yield) as yellow oil. ¹H NMR (400 MHz, CDCl₃) δ 7.86 (d, J=4.0 Hz, 1H), 7.44 (d, J=3.2 Hz, 1H), 7.02-6.75 (m, 3H), 6.48 (s, 1H), 4.16 (q, J=7.2 Hz, 2H), 2.62-2.60 (m, 6H), 1.25-1.21 (m, 12H).

### S73-2: (S)-1-(tert-butoxycarbonyl)-6-(3-fluoro-2-methylphenyl)-4-methyl-2-(thiazol-2-yl)-1,6-dihydropyrimidine-5-carboxylic acid

To a solution of S73-1 (2.5 g, purity 95% purity, 5.17 mmol) in ethanol (50 mL) and tetrahydrofuran (30 mL) was added 2 M sodium hydroxide aqueous solution (18 mL, 36 mmol). After stirred at room temperature for 6 hours, the mixture was concentrated under reduced pressure to remove the volatile. The residue was diluted with ethyl acetate (30 mL) and water (30 mL). 1 M hydrochloride aqueous solution (10 mL) was added to acidify the mixture pH˜5. The organic layer was separated and the aqueous layer was extracted with ethyl acetate (30 mL) twice. The combined organic layers were washed with brine (30 mL), dried over Na₂SO₄₍ₛ₎ and filtered. The filtrate was concentrated to give the title compound (2.1 g, 90% purity, 85% yield) as yellow solids. LC-MS (ESI): mass calcd. for C₂₁H₂₂FN₃O₄S 431.1, m/z found 432.4 [M+H]⁺.

### S73-3: (S)-1-tert-butyl 5-isopropyl 6-(3-fluoro-2-methylphenyl)-4-methyl-2-(thiazol-2-yl)pyrimidine-1,5(6H)-dicarboxylate

To a solution of S73-2 (900 mg, purity 90% purity, 1.88 mmol) in dry acetone (10 mL) was added anhydrous potassium carbonate (400 mg, 2.85 mmol). After stirred at room temperature for 30 minutes, 2-iodopropane (800 mg, 4.71 mmol) was added and continued stirring for another 2 hours. After completion of the reaction, solvent was removed and extracted with ethyl acetate (100 mL) three times. The combined organic layer was dried over anhydrous Na₂SO₄₍ₛ₎ and concentrated under reduced pressure to obtain the crude product, which was purified by silica gel column chromatography (petroleum ether:ethyl acetate=50:1 to 10:1) to give the the title compound (900 mg, 96% purity, 97% yield). LC-MS (ESI): mass calcd. for C₂₄H₂₈FN₃O₄S 473.2, m/z found 474.5 [M+H]⁺.

### S73-4: (S)-1-tert-butyl5-isopropyl6-(3-fluoro-2-methylphenyl)-4-methyl-2-(thiazol-2-yl)pyrimidine-1,5(6H)-dicarboxylate

To a solution of S73-3 (100 mg, 96% purity, 0.205 mmol) in ethyl acetate (20 mL) was added 4 M hydrochloride in ethyl acetate (20 mL). After stirred at room temperature for 2 hours, the reaction mixture was concentrated to give a residue, which was washed with saturated sodium bicarbonate (20 mL) and extracted with ethyl acetate (20 mL) twice. The combined organic layers were washed with saturates brine (200 mL), dried over Na₂SO₄₍ₛ₎ and filtered. The filtrate was concentrated to give the title compound (70 mg, 95% purity from ¹H NMR, 88% yield) as yellow oil. ¹H NMR (400 MHz, CDCl₃) δ 7.78 (d, J=3.2 Hz, 1H), 7.74 (s, 1H), 7.41 (d, J=2.8 Hz, 2H), 7.08-7.05 (m, 2H), 6.91-6.88 (m, 1H), 5.98 (s, 1H), 4.94-4.91 (m, 1H), 2.54 (d, J=2.0 Hz, 3H), 2.51 (s, 3H), 1.18 (d, J=6.4 Hz, 3H), 0.95 (d, J=6.4 Hz, 3H).

### S73: (S)-isopropyl 6-(bromomethyl)-4-(3-fluoro-2-methylphenyl)-2-(thiazol-2-yl)-1,4-dihydropyrimidine-5-carboxylate

This compound was made analogous to H1-1A. LC-MS (ESI): mass calcd. for C₁₉H₁₉BrFN₃O₂S 451.0, m/z found 454.3 [M+H]⁺. ¹H NMR (400 MHz, DMSO-d₆) δ 8.16-7.94 (m, 2H), 7.24-7.03 (m, 3H), 5.77 (s, 1H), 4.95-4.58 (m, 3H), 2.43 (d, J=1.6 Hz, 3H), 1.16 (d, J=6.4 Hz, 3H), 0.92 (d, J=6.4 Hz, 2.4H), 0.86 (d, J=6.4 Hz, 0.6H).

### Compound 152: 4-((cis)-6,6-difluoro-4-(((S)-6-(3-fluoro-2-methylphenyl)-5-(isopropoxycarbonyl)-2-(thiazol-2-yl)-3,6-dihydropyrimidin-4-yl)methyl)hexahydropyrrolo[3,2-b]pyrrol-1(2H)-yl)-2,2-dimethylbutanoic acid

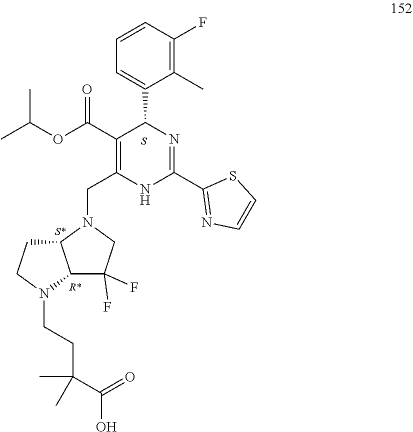

This compound was made from S73 and S9 according to typical method 1 and 3 successively. LC-MS (ESI): mass calcd. for C₃₁H₃₈F₃N₅O₄S 633.2, m/z found 634.4 [M+H]⁺. ¹H NMR (400 MHz, CDCl₃) δ 9.18 (br s, 1H), 7.84 (d, J=3.2 Hz, 1H), 7.44-7.39 (m, 1H), 7.10-7.05 (m, 1H), 7.00-6.97 (m, 1H), 6.90-6.88 (m, 1H), 5.98 (s, 1H), 4.93-4.86 (m, 1H), 4.29-4.25 (m, 1H), 4.13-4.08 (m, 1H), 3.88-3.83 (m, 1H), 3.47-3.38 (m, 3H), 3.20-3.13 (m, 1H), 3.03-2.95 (m, 1H), 2.76-2.67 (m, 1H), 2.58-2.49 (m, 4H), 2.07-1.95 (m, 2H), 1.69-1.65 (m, 2H), 1.28 (s, 6H), 1.17 (d, J=6.4 Hz, 3H), 0.91 (d, J=6.0 Hz, 3H).

### Preparation of Intermediate S63

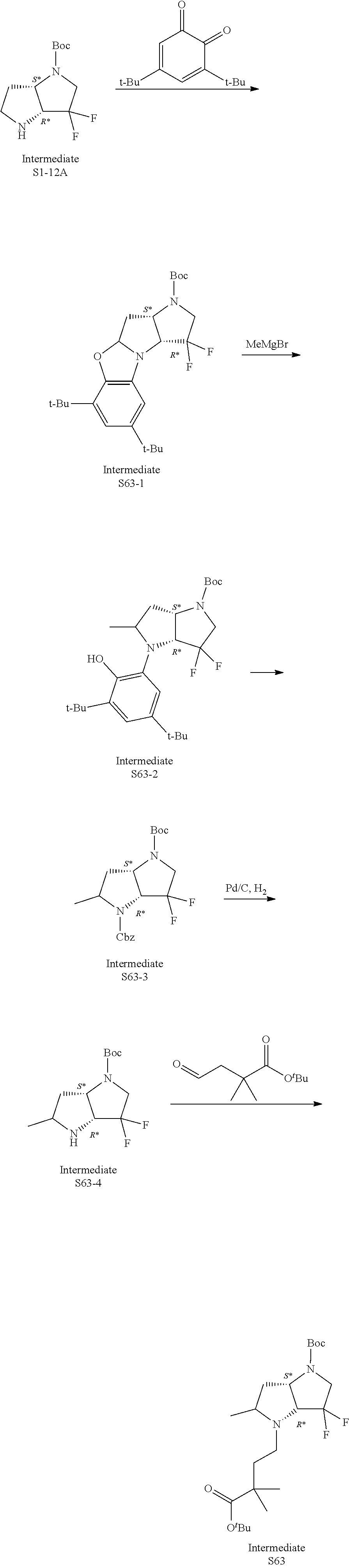

### S63-1: (cis)-tert-Butyl 6,8-di-tert-butyl-3,3-difluoro-2,3,3a,9a,10,10a-hexahydro-1H-benzo[d]pyrrolo[2′,3′:4,5]pyrrolo[2,1-b]oxazole-1-carboxylate

To a solution of S1-12A (500 mg, 95% purity, 1.91 mmol) in 2,2,2-trifluoroethanol (15 mL) was added 3,5-di-tert-butylcyclohexa-3,5-diene-1,2-dione (1.05 g, 4.77 mmol). The mixture was stirred at room temperature and stirred for 1 hour. Then it was heated to 50° C. and stirred overnight. The mixture was cooled to room temperature and concentrated. The residue was purified by silica gel column chromatography (petroleum ether:ethyl acetate=100:1) to give the desired compound (250 mg, 95% purity from ¹H NMR, 28% yield) as white solids. ¹H NMR (400 MHz, CDCl₃) δ 6.89-6.88 (m, 1H), 6.84 (s, 1H), 5.97-5.94 (m, 1H), 4.58-4.50 (m, 1H), 3.99-3.82 (m, 2H), 3.70-3.63 (m, 1H), 2.93-2.79 (m, 1H), 2.25-2.22 (m, 1H), 1.50-1.49 (m, 9H), 1.33 (s, 9H), 1.29 (s, 9H).

### S63-2: (cis)-tert-Butyl 4-(3,5-di-tert-butyl-2-hydroxyphenyl)-3,3-difluoro-5-methylhexahydropyrrolo[3,2-b]pyrrole-1(2H)-carboxylate

To a solution of S63-1 (200 mg, 95% purity, 0.422 mmol) in 1,2-dichloroethane (2 mL) was added 3 M methylmagnesium bromide in tetrahydrofuran (1 mL, 1 mmol) at 0° C. The mixture was stirred at 50° C. and stirred for 30 minutes. The mixture cooled to room temperature and poured into saturated ammonium chloride aqueous solution (10 mL). The mixture was extracted with ethyl acetate (10 mL) twice. The combined organic layers were washed with water brine (10 mL), dried over anhydrous sodium sulfate(s) and filtered. The filtrate was concentrated. The residue was purified by Prep-TLC (petroleum ether:ethyl acetate=10:1) to give the desired compound (160 mg, 77% yield) as white solids. LC-MS (ESI): mass calcd. for C₂₆H₄₀F₂N₂O₃ 466.3, m/z found 467.5 [M+H]⁺.

### S63-3: (cis)-1-Benzyl 4-tert-butyl 6,6-difluoro-2-methyltetrahydropyrrolo[3,2-b]pyrrole-1,4(2H,5H)-dicarboxylate

To a solution of S63-2 (160 mg, 0.343 mmol) in acetonitrile (4 mL) was added 1 M sodium hydroxide aqueous solution (2 mL, 2 mmol) and iodine (96 mg, 0.38 mmol) at 0° C. and stirred for 1 hour. To the mixture was added benzyl carbonochloridate (90 mg, 0.53 mmol). The mixture was stirred at room temperature for 1 hour. The mixture was poured into water (10 mL), extracted with ethyl acetate (10 mL) twice. The combined organic layers were washed with brine (10 mL), dried over anhydrous sodium sulfate(s) and filtered. The filtrate was concentrated. The residue was purified by Prep-TLC (petroleum ether:ethyl acetate=5:1) to give the desired compound (110 mg, 72% purity, 58% yield) as colorless oil. LC-MS (ESI): mass calcd. for C₂₀H₂₆F₂N₂O₄ 396.2, m/z found 397.4 [M+H]⁺.

### S63-4: (cis)-tert-Butyl 3,3-difluoro-5-methylhexahydropyrrolo[3,2-b]pyrrole-1(2H)-carboxylate

To a solution of S63-3 (110 mg, 72% purity, 0.200 mmol) in isopropyl alcohol (10 mL) was added 10% wt. palladium on charcoal (20 mg). The mixture was stirred at room temperature for 2 hours under hydrogen atmosphere (balloon). The mixture was filtered. The filtrate was concentrated to give the title compound (70 mg, 70% purity from ¹H NMR, 93% yield) as colorless oil. ¹H NMR (400 MHz, CDCl₃) δ 4.55-4.32 (m, 1H), 4.04-3.68 (m, 2.3H), 3.52-3.41 (m, 0.7H), 3.30-3.24 (m, 1H), 2.46-2.12 (m, 1H), 2.02-1.74 (m, 1H), 1.47 (s, 9H), 1.18 (d, J=6.4 Hz, 3H).

### S63: (cis)-tert-Butyl 4-(4-(tert-butoxy)-3,3-dimethyl-4-oxobutyl)-3,3-difluoro-5-methylhexahydropyrrolo[3,2-b]pyrrole-1(2H)-carboxylate

This intermediate was made from S63-4 and tert-butyl 2,2-dimethyl-4-oxobutanoate according to typical method 5. Purified by C18 column (acetonitrile:water=50% to 80%) to give the desired compound (80 mg, 84% purity, 83% yield) as colorless oil. LC-MS (ESI): mass calcd. for C₂₂H₃₈F₂N₂O₄ 432.3, m/z found 433.2 [M+H−100]⁺.

### Compound 153: 4-((cis)-4-(((S)-5-(Ethoxycarbonyl)-6-(3-fluoro-2-methylphenyl)-2-(thiazol-2-yl)-3,6-dihydropyrimidin-4-yl)methyl)-6,6-difluoro-2-methylhexahydropyrrolo[3,2-b]pyrrol-1(2H)-yl)-2,2-dimethylbutanoic acid

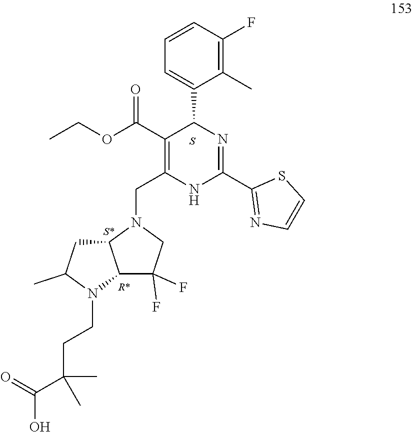

This compound was made from H2-1A and S63 according to typical method 1 and 3 successively. LC-MS (ESI): mass calcd. for C₃₁H₃₈F₃N₅O₄S 633.3, m/z found 634.3 [M+H]⁺. ¹H NMR (400 MHz, CDCl₃) δ 9.33-9.22 (m, 1H), 7.83-7.81 (m, 1H), 7.41-7.40 (m, 1H), 7.10-7.05 (m, 1H), 6.99-6.97 (m, 1H), 6.93-6.88 (m, 1H), 6.01-6.00 (m, 1H), 4.33-4.28 (m, 0.7H), 4.14-4.00 (m, 3.6H), 3.88-3.77 (m, 1.7H), 3.62-3.57 (m, 0.7H), 3.41-3.35 (m, 0.3H), 3.30-3.23 (m, 1H), 3.14-3.03 (m, 1.7H), 2.95-2.80 (m, 1.3H), 2.64-2.54 (m, 3.7H), 2.09-2.01 (m, 1.3H), 1.95-1.89 (m, 1H), 1.81-1.77 (m, 0.7H), 1.65-1.56 (m, 0.3H), 1.29-1.25 (m, 6H), 1.20-1.18 (m, 3H), 1.13-1.09 (m, 3H).

### Preparation of Intermediate S72

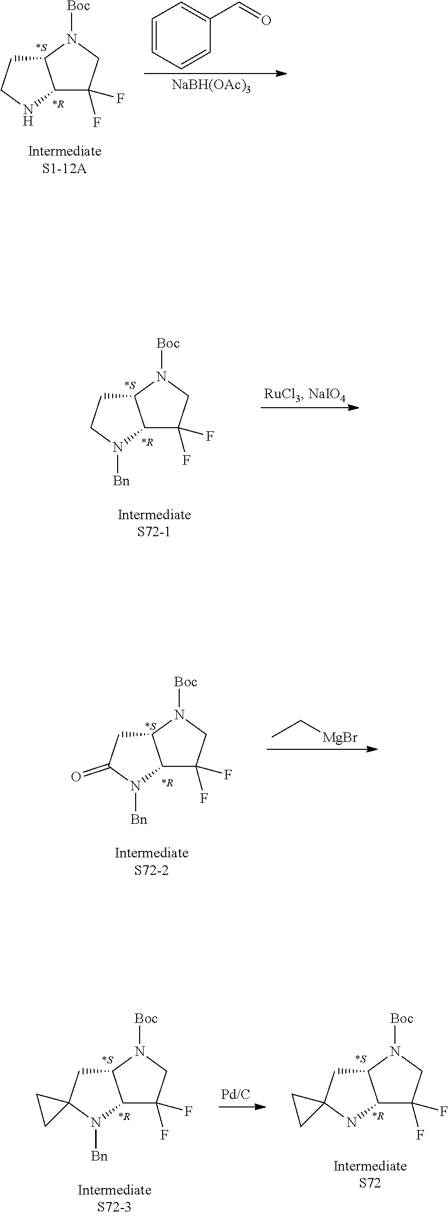

### S72-1: (cis)-tert-butyl 4-benzyl-3,3-difluorohexahydropyrrolo[3,2-b]pyrrole-1(2H)-carboxylate

This compound was made from S1-12A and benzaldehyde according to typical method 5. LC-MS (ESI): mass calcd. for C₁₈H₂₄F₂N₂O₂ 338.2, m/z found 339.5 [M+H]⁺.

### S72-2: (cis)-tert-butyl 4-benzyl-3,3-difluoro-5-oxohexahydropyrrolo[3,2-b]pyrrole-1(2H)-carboxylate

To a solution of S72-1 (2.00 g, 100% purity, 5.91 mmol) in carbon tetrachloride (30 mL) was added ruthenium(III) chloride (200 mg, 0.964 mmol), sodium periodate (4.00 g, 18.7 mmol) and water (10 mL). The mixture was stirred at 0° C. for 30 minutes. The mixture was diluted with dichloromethane (50 mL) and filtered. The filtrate was poured into water (80 mL) and extracted with dichloromethane (50 mL) for three times. The combined organic layers were washed with brine (150 mL), dried over anhydrous Na₂SO₄ and filtered. The filtrate was concentrated to give a residue. The residue was purified by silica gel column chromatography (petroleum ether:ethyl acetate=1:0 to 3:1) to give the title compound (1.40 g, 100% purity by LCMS, 67% yield) as colorless oil. LC-MS (ESI): mass calcd. for C₁₈H₂₂F₂N₂O₃ 352.2, m/z found 353.1 [M+H]⁺.

### S72-3: (cis)-tert-butyl 1′-benzyl-6′,6′-difluorotetrahydro-1′H-spiro[cyclopropane-1,2′-pyrrolo[3,2-b]pyrrole]-4′(5′H)-carboxylate

To a solution of S72-2 (1.40 g, 100% purity, 3.97 mmol) and titanium tetraisopropanolate (1.20 g, 4.22 mmol) in tetrahydrofuran (20 mL) was added 1 M ethylmagnesium bromide in tetrahydrofuran (12 mL, 12.0 mmol) at 0° C. The mixture was stirred at room temperature overnight. The reaction mixture was poured into water (30 mL) and extracted with dichloromethane (30 mL) for three times. The combined organic layers were washed with brine (80 mL), dried over anhydrous Na₂SO₄ and filtered. The filtrate was concentrated to give a residue. The residue was purified by silica gel column chromatography (petroleum ether:ethyl acetate=1:0 to 5:1) to give the title compound (750 mg, 90% purity by LCMS, 47% yield) as colorless oil. LC-MS (ESI): mass calcd. For C₂₀H₂₆F₂N₂O₂ 364.2, m/z found 365.2 [M+H]⁺.

### S72: (cis)-tert-butyl 6′,6′-difluorotetrahydro-1′H-spiro[cyclopropane-1,2′-pyrrolo[3,2-b]pyrrole]-4′(5′H)-carboxylate

To a solution of S72-3 (500 mg, 90% purity, 1.24 mmol) in ethanol (10 mL) was added 10% wt. palladium on charcoal (100 mg, 0.094 mmol). The mixture was stirred for 10 minutes under hydrogen atmosphere (1 atm). The mixture was filtered and the filtrate was concentrated to give a residue. The residue was purified by silica gel column chromatography (petroleum ether:ethyl acetate=1:0 to 3:1) to give the title compound (100 mg, 90% purity by LCMS, 27% yield,) as colorless oil. LC-MS (ESI): mass calcd. for C₁₃H₂₀F₂N₂O₂ 274.2, m/z found 275.3 [M+H]⁺.

### Compound 154: 4-((cis)-4′-(((S)-5-(ethoxycarbonyl)-6-(3-fluoro-2-methylphenyl)-2-(thiazol-2-yl)-3,6-dihydropyrimidin-4-yl)methyl)-6′,6′-difluorohexahydro-1′H-spiro[cyclopropane-1,2′-pyrrolo[3,2-b]pyrrol]-1′-yl)-2,2-dimethylbutanoic acid

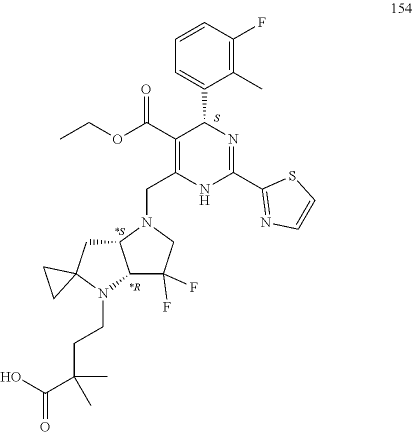

This compound was made from S72 and tert-butyl 2,2-dimethyl-4-oxobutanoate and H2-1A according typical method 5, 1 and 3 successively. LC-MS (ESI): mass calcd. for C₃₂H₃₈F₃N₅O₄S 645.3, m/z found 646.4 [M+H]⁺. H NMR (400 MHz, CDCl₃): δ 9.31 (br s, 1H), 7.80 (d, J=2.8 Hz, 1H), 7.40 (d, J=2.8 Hz, 1H), 7.08-7.03 (m, 1H), 6.98-6.96 (m, 1H), 6.92-6.87 (m, 1H), 6.01 (s, 1H), 4.34-4.31 (m, 1H), 4.07-4.00 (m, 3H), 3.89-3.86 (m, 1H), 3.81-3.73 (m, 1H), 3.35-3.28 (m, 1H), 2.92-2.84 (m, 2H), 2.64-2.57 (m, 1H), 2.53 (s, 3H), 2.23-2.18 (m, 1H), 1.81-1.66 (m, 2H), 1.61-1.58 (m, 1H), 1.32-1.29 (m, 1H), 1.26 (s, 6H), 1.10 (t, J=7.2 Hz, 3H), 0.95-0.88 (m, 1H), 0.84-0.79 (m, 1H), 0.67-0.61 (m, 1H).

### Compound 155: 3-(((cis)-4-(((S)-5-(Ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)methyl)bicyclo[1.1.1]pentane-1-carboxylic acid

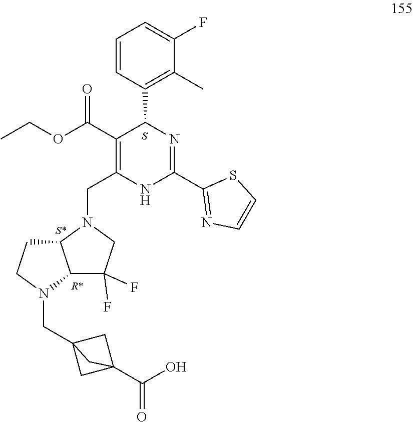

This compound was made from compound 103 and methyl 3-formylbicyclo[1.1.1]pentane-1-carboxylate according to typical method 5 and 4 successively. LC-MS (ESI): mass calcd. for C₃₁H₃₄F₃N₅O₄S 629.7, m/z found 630.2 [M+H]⁺. ¹H NMR (400 MHz, CD₃OD) δ 7.95 (d, J=3.2 Hz, 1H), 7.73 (d, J=2.8 Hz, 1H), 7.20-7.11 (m, 2H), 6.98-6.93 (m, 1H), 5.99 (s, 1H), 4.30 (d, J=17.2 Hz, 1H), 4.17 (d, J=16.8 Hz, 1H), 4.07 (q, J=7.2 Hz, 2H), 3.90-3.85 (m, 1H), 3.44-3.37 (m, 3H), 3.19-3.14 (m, 0.3H), 3.05-3.00 (m, 0.7H), 2.97 (d, J=13.6 Hz, 1H), 2.68 (d, J=13.2 Hz, 1H), 2.58-2.57 (m, 0.7H), 2.53 (m, 3H), 2.37-2.34 (m, 0.3H), 2.06-1.96 (m, 8H), 1.15 (t, J=7.2 Hz, 3H).

### Preparation of Intermediate S74

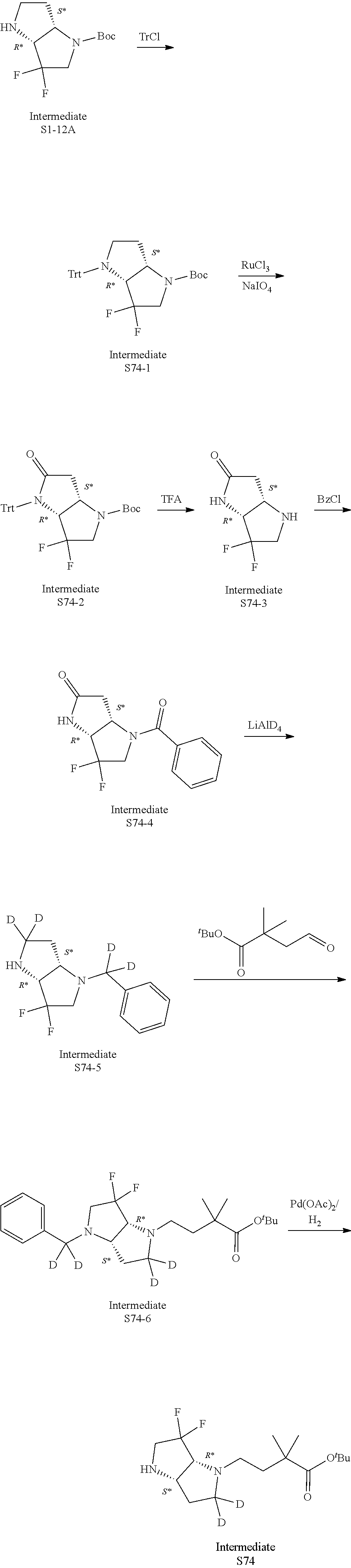

### S74-1: (cis)-tert-Butyl 3,3-difluoro-4-tritylhexahydropyrrolo[3,2-b]pyrrole-1(2H)-carboxylate

To a solution of S1-12A (1.5 g, 95% purity, 5.74 mmol) and N,N-diisopropylethylamine (2.22 g, 17.2 mmol) in dichloromethane (10 mL) was added (chloromethanetriyl)tribenzene (2.40 g, 8.61 mmol) at room temperature. After stirred at room temperature overnight, the mixture was quenched with ice water (30 mL), extracted with ethyl acetate (20 mL) for three times. The combined organic layers were dried over Na₂SO₄₍ₛ₎ and filtered. The filtrate was concentrated to give a residue, which was purified by C18 column (acetonitrile:water=50% to 100%) to give the title compound (2.8 g, 90% purity from ¹H NMR, 89% yield) as white solids. ¹H NMR (400 MHz, CDCl₃) δ 7.59-7.58 (m, 5.5H), 7.31-7.27 (m, 7H), 7.20-7.17 (m, 2.5H), 4.23-4.15 (m, 1H), 3.78-3.55 (m, 3H), 3.38-3.24 (m, 1H), 3.13-3.02 (m, 1H), 1.48-1.47 (m, 2H), 1.39-1.34 (m, 9H).

### S74-2: (cis)-tert-Butyl 3,3-difluoro-5-oxo-4-tritylhexahydropyrrolo[3,2-b]pyrrole-1(2H)-carboxylate

To a solution of S74-1 (2.8 g, 90% purity, 5.137 mmol) in ethyl acetate (15 mL) and water (15 mL) were added ruthenium trichloride (0.499 g, 2.41 mmol) and sodium periodate (2.50 g, 11.7 mmol) at room temperature. After stirred at 20° C. overnight, the mixture was quenched with saturated sodium bicarbonate (50 mL). The aqueous phase was separated and extracted with ethyl acetate (30 mL) twice. The combined organic phases were washed with saturated sodium bicarbonate solution (10 mL) and brine (10 mL), dried over Na₂SO₄₍ₛ₎. The mixture was filtered and concentrated in vacuo to give a crude product, which was purified by C18 column (acetonitrile:water=50% to 90%) to give the desired product (1.7 g, 90% purity from ¹H NMR, 59% yield) as yellow solids, ¹H NMR (400 MHz, CDCl₃) δ 7.30-7.23 (m, 15H), 4.88-4.76 (m, 0.5H), 4.71-4.60 (m, 0.5H), 4.45-4.29 (m, 1H), 3.94-3.76 (m, 1H), 3.69-3.51 (m, 1H), 2.94-2.80 (m, 1H), 2.64-2.53 (m, 1H), 1.46 (s, 9H).

### S74-3: (cis)-6,6-Difluorohexahydropyrrolo[3,2-b]pyrrol-2(1H)-one

To a solution of S74-2 (1.7 g, 90% purity, 3.03 mmol) in dichloromethane (5 mL) was added trifluoroacetic acid (5 mL) at 0° C. After stirred at room temperature overnight, the reaction mixture was concentrated under reduced pressure to give the crude product, which was diluted with water (10 mL), extracted with tert-butyl methyl ether (10 mL) for three times.

Then the aqueous phase were basified with 1 M sodium carbonate aqueous solution to pH 9˜10 and extracted with ethyl acetate (20 mL) for three times. The combined organic phases were dried over Na₂SO₄₍ₛ₎, filtered and evaporated to give the title compound (400 mg, 90% purity from ¹H NMR, 73% yield) as light yellow oil. ¹H NMR (400 MHz, CDCl₃) δ 5.91 (s, 1H), 4.25-4.21 (m, 1H), 4.04-3.99 (m, 1H), 3.21-3.09 (m, 2H), 2.79-2.72 (m, 1H), 2.36-2.31 (m, 1H).

### S74-4: (cis)-4-Benzoyl-6,6-difluorohexahydropyrrolo[3,2-b]pyrrol-2(1H)-one

To a solution of S74-3 (400 mg, 90% purity, 2.22 mmol) in dichloromethane (3 mL) was added N,N-diisopropylethylamine (1.5 g, 10.1 mmol) and benzoyl chloride (780 mg, 4.41 mmol) at room temperature. After stirred at 25° C. under nitrogen atmosphere for 1 hour, the mixture was quenched with saturated ammonium chloride solution (20 mL), extracted with ethyl acetate (20 mL) for three times. The combined organic phase were washed with brine (10 mL) for three times, dried over Na₂SO₄₍ₛ₎, filtered and concentrated under reduced pressure to give a residue, which was purified by C18 (acetonitrile:water=20% to 85%) to give the desired compound (520 mg, 90% purity from ¹H NMR, 79% yield) as yellow oil. ¹H NMR (400 MHz, CDCl₃) δ 7.53-7.43 (m, 5H), 6.26 (s, 1H), 5.15 (s, 1H), 4.32-4.28 (m, 1H), 4.06-3.81 (m, 2H), 2.96-2.85 (m, 1H), 2.68-2.58 (m, 1H).

### S74-5: (cis)-1-Benzyl-3,3-difluorooctahydropyrrolo[3,2-b]pyrrole-d4

To a solution of S74-4 (520 mg, 90% purity, 1.76 mmol) in tetrahydrofuran (12 mL) was added lithium aluminium hydride-d₄ (600 mg, 14.3 mol) at 0° C. After stirred at 70° C. for 8 hours, the mixture was quenched with saturated sodium hydroxide solution (20 mL) at 0° C., extracted with ethyl acetate (20 mL) for three times. The combined organic phases were washed with brine (20 mL), dried over Na₂SO₄₍ₛ₎, filtered and evaporated to give the title compound 470 mg, 90% purity from ¹H NMR, 99% yield) as light yellow oil. ¹H NMR (400 MHz, CDCl₃) δ 7.33-7.27 (m, 5H), 3.83-3.76 (m, 1H), 3.48-3.45 (m, 1H), 3.14-3.08 (m, 1H), 2.69-2.58 (m, 1H), 1.84-1.81 (m, 1H), 1.60-1.56 (m, 1H).

### S74-6: (cis)-tert-butyl 4-(4-benzyl-6,6-difluorohexahydropyrrolo[3,2-b]pyrrol-1(2H)-yl)-2,2-dimethylbutanoate-d4

This compound was made from S74-5 and tert-butyl 2,2-dimethyl-4-oxobutanoate according to typical method 5. LC-MS (ESI): mass calcd. for C₂₃H₃₀D₄F₂N₂O₂ 412.3, m/z found 413.3 [M+H]⁺.

### S74: (cis)-tert-butyl 4-(6,6-difluorohexahydropyrrolo[3,2-b]pyrrol-1(2H)-yl)-2,2-dimethylbutanoate-d2

To a solution of S74-6 (100 mg, 97.2% purity, 0.236 mmol) and palladium acetate (50 mg, 0.223 mmol) in isopropyl alcohol (5 mL) was added activated carbon (80 mg, 6.67 mmol). The reaction was stirred at 50° C. under hydrogen atmosphere of balloon for 2 hours, then it was filtered and the filtrate was concentrated under reduced pressure to give the title compound (70 mg, 80% purity from ¹H NMR, 74% yield) as brown oil. ¹H NMR (400 MHz, CDCl₃) δ 4.03-3.97 (m, 1H), 3.24-3.13 (m, 1H), 3.06-2.99 (m, 1H), 2.85-2.79 (m, 2H), 2.37-2.31 (m, 1H), 2.16-2.10 (m, 1H), 1.75-1.67 (m, 3H), 1.44 (s, 9H), 1.14 (s, 6H).

### Compound 157: 4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydropyrrolo[3,2-b]pyrrol-1(2H)-yl)-2,2-dimethylbutanoic acid-d2

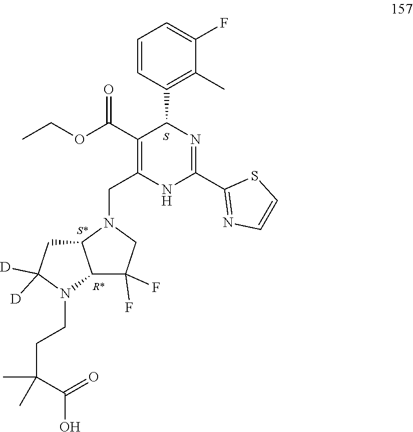

This compound was made from H2-1A and S74 according to typical method 1 and 3 successively. LC-MS (ESI): mass calcd. for C₃₀H₃₄D₂F₃N₅O₄S 621.3, m/z found 622.3 [M+H]⁺. ¹H NMR (400 MHz, CDCl₃) δ 9.24 (s, 1H), 7.84 (d, J=3.2 Hz, 1H), 7.40 (d, J=3.2 Hz, 1H), 7.10-7.04 (m, 1H), 6.98-6.96 (m, 1H), 6.93-6.88 (m, 1H), 6.00 (s, 1H), 4.28 (d, J=17.6 Hz, 1H), 4.11 (d, J=17.2 Hz, 1H), 4.08-3.99 (m, 2H), 3.86-3.81 (m, 1H), 3.46-3.33 (m, 2H), 3.17-3.10 (m, 1H), 3.00-2.92 (m, 1H), 2.72-2.68 (m, 1H), 2.53 (s, 3H), 2.04-1.91 (m, 2.7H), 1.69-1.63 (m, 1.3H), 1.28 (s, 3H), 1.27 (s, 3H), 1.10 (t, J=7.2 Hz, 3H).

### Preparation of Intermediate S77

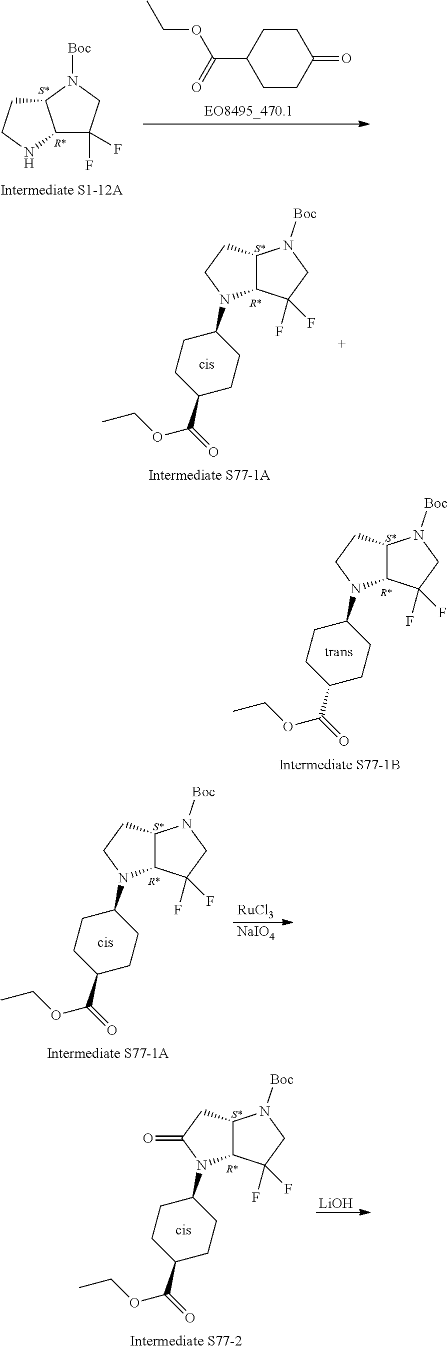

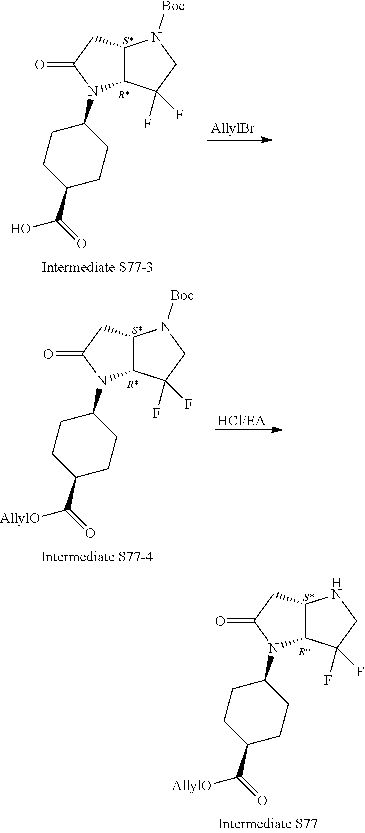

### S77-1A/B: (cis)-tert-butyl cis-4-(ethoxycarbonyl)cyclohexyl)-3,3-difluorohexahydropyrrolo[3,2-b]pyrrole-1(2H)-carboxylate and (cis)-tert-butyl trans-4-(ethoxycarbonyl)cyclohexyl)-3,3-difluorohexahydropyrrolo[3,2-b]pyrrole-1(2H)-carboxylate

To a solution of S1-12A (580 mg, 2.34 mmol, 95% purity) and ethyl 4-oxocyclohexanecarboxylate (425 mg, 2.50 mmol) in dichloromethane (30 mL) was added acetic acid (1 mL, 17.5 mmol). After stirred for 1 hour at room temperature, sodium triacetoxyborohydride (2 g, 10.1 mmoL) was added. The mixture was stirred at room temperature overnight. Then the reaction mixture was quenched with sodium bicarbonate solution (30 mL) and dichloromethane (50 mL). The organic solution was washed with brine (20 mL), dried over sodium sulfate (s) and filtered. The filtrate was concentrated and purified by column chromatography on silica gel (hexane:ethyl acetate=10:1) to afford the desired S77-1A (400 mg, 42% yield, 95% purity by ¹H NMR) and S77-1B (230 mg, 24.4% yield, 95% purity by ¹H NMR) as yellow oil.

S77-1A: ¹H NMR (400 MHz, CDCl₃) δ 4.45-4.35 (m, 1H), 4.14 (q, J=7.2 Hz, 2H), 3.82-3.48 (m, 3H), 3.03-2.98 (m, 1H), 2.75-2.52 (m, 3H), 2.17-1.67 (m, 6H), 1.55-1.49 (m, 4H), 1.46 (s, 9H), 1.26 (t, J=7.2 Hz, 3H).

S77-1B: ¹H NMR (400 MHz, CDCl₃) δ 4.49-4.37 (m, 1H), 4.11 (q, J=7.2 Hz, 2H), 3.88-3.48 (m, 3H), 3.07-3.02 (m, 1H), 2.77-2.57 (m, 2H), 2.24-2.17 (m, 1H), 2.10-1.80 (m, 6H), 1.43 (s, 9H), 1.32-1.23 (m, 7H).

### S77-2: (cis)-tert-butyl cis-4-(ethoxycarbonyl)cyclohexyl)-3,3-difluoro-5-oxohexahydropyrrolo[3,2-b]pyrrole-1(2H)-carboxylate

To a solution of S77-1A (400 mg, 0.995 mmol, 95% purity) in ethyl acetate (20 mL) and water (10 mL) was added ruthenium (III) chloride trihydrate (79 mg, 0.30 mmol). Then sodium periodate (1.07 g, 4.98 mmoL) was added at 0° C. After stirred at 0° C. for 10 minutes, the reaction mixture was quenched with saturated sodium thiosulfate solution (20 mL) and the mixture was extracted by ethyl acetate (90 mL) for three times. The combined organic layers were washed with brine (30 mL), dried over sodium sulfate (s) and filtered. The filtrate was concentrated and the residue was purified by silica gel column chromatography (dichloromethane:methanol=20:1) to afford the desired product (200 mg, 48% yield, 100% purity) as white solids. LC-MS (ESI): mass calcd. for C₂₀H₃₀F₂N₂O₅ 416.2, m/z found 417.4 [M+H]⁺.

### S77-3: cis-4-((cis)-4-(tert-butoxycarbonyl)-6,6-difluoro-2-oxohexahydropyrrolo[3,2-b]pyrrol-1(2H)-yl)cyclohexanecarboxylic acid

To a mixture of S77-2 (200 mg, 0.48 mmol, 100% purity) in methanol (2 mL) and water (2 mL) was added lithium hydroxide monohydrate (32 mg, 0.792 mmol). The reaction mixture was stirred overnight at room temperature. Then the reaction mixture was acidified by 3 N hydrochloride to pH=3 and then the mixture was extracted with ethyl acetate (30 mL). The organic solution was washed with brine (20 mL), dried over sodium sulfate (s) and filtered. The filtrate was concentrated to get the crude product (170 mg, 87% yield, 90% purity) as white solids. LC-MS (ESI): mass calcd. for C₁₈H₂₆F₂N₂O₅ 388.2, m/z found 389.4 [M+H]⁺.

### S77-4: (cis)-tert-butyl cis-4-((allyloxy)carbonyl)cyclohexyl)-3,3-difluoro-5-oxohexahydropyrrolo[3,2-b]pyrrole-1(2H)-carboxylate

To a mixture of S77-3 (170 mg, 0.416 mmol, 90% purity) in N,N-dimethylformamide (2 mL) was added allyl bromide (70 mg, 0.583 mmol) and potassium carbonate (181 mg, 1.31 mmol). The mixture was stirred at room temperature for 3 hours under nitrogen atmosphere. Then the reaction mixture was poured into water (10 mL) and extracted with ethyl acetate (30 mL). The organic solution was washed with brine (10 mL), dried over sodium sulfate (s) and filtered. The solution was concentrated to get the crude, which was purified by C18 column (acetonitrile:water=10% to 70%) to afford the desired product (190 mg, 96% yield, 90% purity) as yellow oil. LC-MS (ESI): R_{T}=1.70 min, mass calcd. for C₂₁H₃₀F₂N₂O₅ 428.2, m/z found 429.4 [M+H]⁺.

### S77: cis-allyl 4-((cis)-6,6-difluoro-2-oxohexahydropyrrolo[3,2-b]pyrrol-1(2H)-yl)cyclohexanecarboxylate

To a solution of S77-4 (190 mg, 0.4 mmol, 90% purity) in ethyl acetate (2 mL) was added 6 M hydrochloride in ethyl acetate (5 mL). The reaction mixture was stirred for 3 hours at room temperature. The reaction mixture was poured into saturated sodium bicarbonate solution (10 mL) and the mixture was extracted by ethyl acetate (50 mL) for three times. The combined organic layers were washed with brine (30 mL), dried over sodium sulfate (s) and filtered. The filtrate was concentrated to give the title product (100 mg, 76% yield, 100% purity) as yellow oil. LC-MS (ESI): mass calcd. for C₁₆H₂₂F₂N₂O₃ 328.1, m/z found 329.3 [M+H]⁺.

### Compound 158A: cis-4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluoro-2-oxohexahydropyrrolo[3,2-b]pyrrol-1(2H)-yl)cyclohexanecarboxylic acid

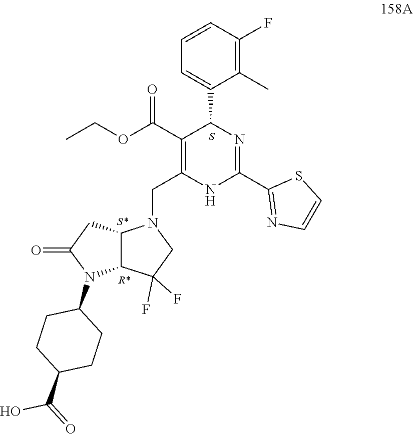

This compound was made from S77 and H2-1A according to typical method 1 and 2 successively. LC-MS (ESI): mass calcd. for C₃₁H₃₄F₃N₅O₅S 645.2, m/z found 646.2 [M+H]⁺. ¹H NMR (400 MHz, CDCl₃) δ 9.05 (s, 1H), 7.81 (d, J=2.8 Hz, 1H), 7.40 (d, J=3.2 Hz, 1H), 7.10-7.05 (m, 1H), 6.98-6.96 (m, 1H), 6.93-6.88 (m, 1H), 6.00 (s, 1H), 4.32-4.25 (m, 2H), 4.09-4.01 (m, 3H), 3.95-3.82 (m, 2H), 3.33-3.26 (m, 1H), 3.10-3.00 (m, 1H), 2.72-2.71 (m, 1H), 2.62-2.60 (m, 2H), 2.52 (s, 3H), 2.31-2.28 (m, 2H), 1.91-1.84 (m, 2H), 1.71-1.57 (m, 4H), 1.10 (t, J=7.2 Hz, 3H).

### Compound 158B: trans-4-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluoro-2-oxohexahydropyrrolo[3,2-b]pyrrol-1(2H)-yl)cyclohexanecarboxylic acid

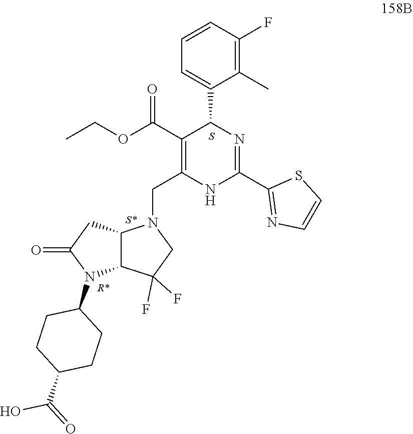

This compound was made analogous to 158A from S77-1B. LC-MS (ESI): mass calcd. for C₃₁H₃₄F₃N₅O₅S 645.2, m/z found 646.2 [M+H]⁺. ¹H NMR (400 MHz, CDCl₃) δ 9.05 (s, 1H), 7.81 (d, J=2.8 Hz, 1H), 7.41 (d, J=2.8 Hz, 1H), 7.11-7.05 (m, 1H), 6.99-6.97 (m, 1H), 6.93-6.89 (m, 1H), 6.01 (s, 1H), 4.35-4.23 (m, 2H), 4.08-4.00 (m, 3H), 3.88-3.77 (m, 2H), 3.36-3.29 (m, 1H), 3.09-3.00 (m, 1H), 2.63-2.61 (m, 2H), 2.53 (s, 3H), 2.37-2.31 (m, 1H), 2.18-2.03 (m, 3H), 1.86-1.77 (m, 2H), 1.73-1.57 (m, 3H), 1.10 (t, J=6.8 Hz, 3H).

### Compound 159: 3-((cis)-4-((5-(ethoxycarbonyl)-6-(6-fluoro-2-methylpyridin-3-yl)-2-(thiazol-2-yl)-3,6-dihydropyrimidin-4-yl)methyl)-6,6-difluorohexahydropyrrolo[3,2-b]pyrrol-1(2H)-yl)-2,2-dimethylpropanoic acid (Single Diastereomer)

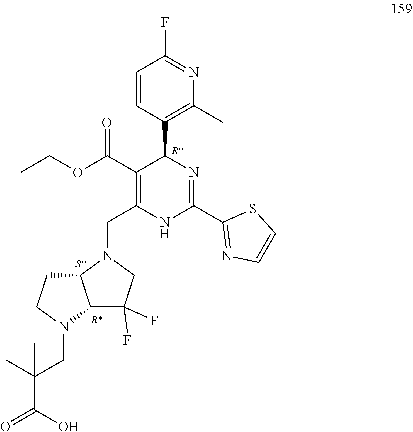 [SMILES: CCOC(=O)C1=C(CN2CC(F)(F)[C@H]3[C@@H]2CCN3CC(C)(C)C(=O)O)NC(c2nccs2)=N[C@@H]1c1ccc(F)nc1C]

This compound was made from H20-1A and S16 according to typical method 1 and 2 successively. Purified by Prep. HPLC (Column: Waters Xbrige C18 (5 μm 19*150 mm), Mobile phase A: water (0.1% ammonium bicarbonate), Mobile phase B: acetonitrile, UV: 214 nm, Flow rate: 15 mL/min, Gradient: 20-95% (% B)) to give the title compound (27.7 mg, 47% purity, 49.4% yield) as yellow solids. LC-MS (ESI): mass calcd. for C₂₈H₃₃F₃N₆O₄S, 606.2, m/z found 607.3 [M+H]⁺. ¹H NMR (400 MHz, CDCl₃+ one drop of D₂O) δ 7.87 (d, J 2.8 Hz, 1H), 7.54 (t, J 2.8 Hz, 1H), 7.45 (d, J 2.8 Hz, 1H), 6.67 (d, J 8.0 Hz, 1H), 5.99 (s, 1H), 4.36 (d, J=17.6 Hz, 1H), 4.11-3.98 (m, 3H), 3.77-3.67 (m, 2H), 3.49-3.43 (m, 1H), 3.33-3.26 (m, 1H), 2.97-2.84 (m, 4H), 2.79 (s, 3H), 1.94 (m, 2H), 1.27 (d, J 9.6 Hz, 6H) 1.13 (t, J 6.8 Hz, 3H).

### Preparation of Intermediate S37

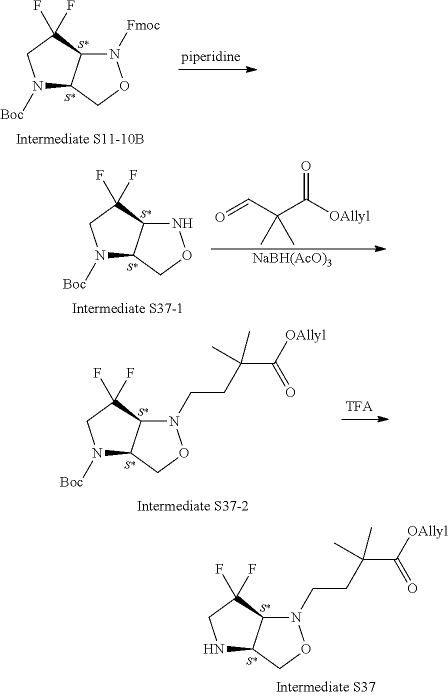

### S37-1: (cis)-tert-Butyl 6,6-difluorotetrahydro-1H-pyrrolo[3,2-c]isoxazole-4(5H)-carboxylate

To a solution of S11-10B (3.50 g, 90% purity, 6.67 mmol) in N,N-dimethylethanamine (35 mL) was added piperidine (2.80 g, 32.9 mmol). After stirred at room temperature for 4 hours, the mixture was poured into water (100 mL) and extracted with dichloromethane (50 mL) for three times. The combined organic layers were washed with brine (100 mL) and concentrated to give a residue, which was purified by silica gel column chromatography (petroleum ether:ethyl acetate=4:1) to afford the desired compound (1.70 g, 90% purity from ¹H NMR, 91% yield) as yellow oil. 1H NMR (300 MHz, CDCl₃) δ 5.60-5.56 (m, 1H), 4.99-4.86 (m, 1H), 4.38-4.24 (m, 1H), 4.08-3.91 (m, 1H), 3.64-3.49 (m, 2H), 1.52-1.51 (m, 9H).

### S37-2: (cis)-tert-Butyl 1-(4-(allyloxy)-3,3-dimethyl-4-oxobutyl)-6,6-difluorotetrahydro-1H-pyrrolo[3,2-c]isoxazole-4(5H)-carboxylate

This intermediate was made from S37-2 and allyl 2,2-dimethyl-4-oxobutanoate analogous to S11-12. Purified by silica gel column chromatography (petroleum ether:ethyl acetate=4:1) to afford the desired compound (2.60 g, 90% purity from ¹H NMR, 94% yield) as yellow oil. ¹H NMR (400 MHz, CDCl₃) δ 5.96-5.87 (m, 1H), 5.35-5.30 (m, 1H), 5.25-5.22 (m, 1H), 4.53-4.71 (m, 1H), 4.57-4.56 (m, 2H), 4.16-4.10 (m, 1H), 4.02-3.61 (m, 3H), 3.44-3.36 (m, 1H), 2.91-2.78 (m, 2H), 1.91 (t, J=8.0 Hz, 2H), 1.46 (s, 9H), 1.24 (s, 3H), 1.23 (s, 3H).

### Compound 160: 4-((cis)-6,6-difluoro-4-(((S)-6-(3-fluoro-2-methylphenyl)-5-(methoxycarbonyl)-2-(thiazol-2-yl)-3,6-dihydropyrimidin-4-yl)methyl)hexahydro-1H-pyrrolo[3,2-c]isoxazol-1-yl)-2,2-dimethylbutanoic acid (Single Diastereomer)

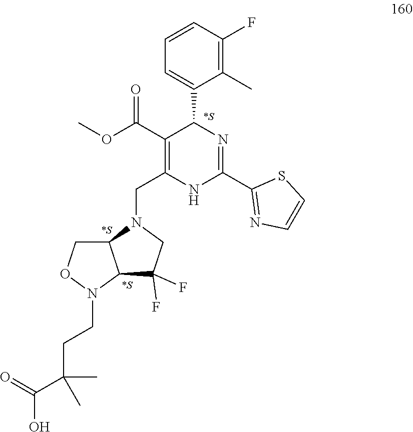 [SMILES: COC(=O)C1=C(CN2CC(F)(F)[C@@H]3[C@H]2CON3CCC(C)(C)C(=O)O)NC(c2nccs2)=N[C@H]1c1cccc(F)c1C]

This compound was made from H4-1B and S37 according to typical coupling method 1 and typical method 2 successively. Purified by C18 column (acetonitrile:water (0.1% ammonium bicarbonate=5% to 80%) to give the title compound (22 mg, 97% purity, 50% yield) as yellow solids. LC-MS (ESI): mass calcd. for C₂₈H₃₂F₃N₅O₅S 607.2, m/z found 608.3 [M+H]⁺. ¹H NMR (400 MHz, CDCl₃) δ 9.24 (s, 1H), 7.82 (d, J=3.2 Hz, 1H), 7.41 (d, J=3.2 Hz, 1H), 7.10-7.05 (m, 1H), 6.96-6.89 (m, 2H), 5.99 (s, 1H), 4.32-4.22 (m, 3H), 4.05-3.98 (m, 2H), 3.75-3.62 (m, 1H), 3.59 (s, 3H), 3.50-3.39 (m, 1H), 3.11-3.05 (m, 1H), 2.92-2.85 (m, 2H), 2.54 (s, 3H), 1.96-1.86 (m, 2H), 1.26 (s, 3H), 1.24 (s, 3H).

### Compound 161: (cis)-4-(4-((6-(3,4-difluoro-2-methylphenyl)-5-(methoxycarbonyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

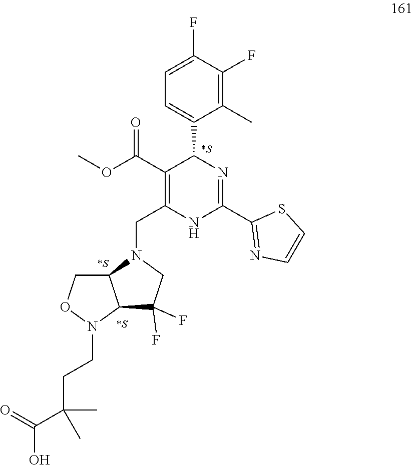 [SMILES: COC(=O)C1=C(CN2CC(F)(F)[C@@H]3[C@H]2CON3CCC(C)(C)C(=O)O)NC(c2nccs2)=N[C@H]1c1ccc(F)c(F)c1C]

This compound was made from H6-1B and S37 according to typical coupling method 1 and typical method 2 successively. Purified by C18 column (acetonitrile:water (0.1% ammonium bicarbonate=5% to 80%) to give the title compound (33.2 mg, 98.0% purity, 76% yield) as yellow solids. LC-MS (ESI): mass calcd. for C₂₈H₃₁F₄N₅O₅S 625.2, m/z found 626.2 [M+H]⁺. ¹H NMR (400 MHz, CDCl₃) δ 9.27 (s, 1H), 7.82 (d, J=3.2 Hz, 1H), 7.42 (d, J=3.2 Hz, 1H), 6.93-6.84 (m, 2H), 5.92 (s, 1H), 4.26-4.23 (m, 3H), 4.05-3.98 (m, 2H), 3.66-3.62 (m, 1H), 3.60 (s, 3H), 3.49-3.38 (m, 1H), 3.01-2.99 (m, 1H), 2.90-2.84 (m, 2H), 2.57 (s, 3H), 1.95-1.84 (m, 2H), 1.23 (s, 3H), 1.22 (s, 3H).

### Compound 162: (cis)-4-(4-((6-(2-chloro-3,4-difluorophenyl)-5-(methoxycarbonyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

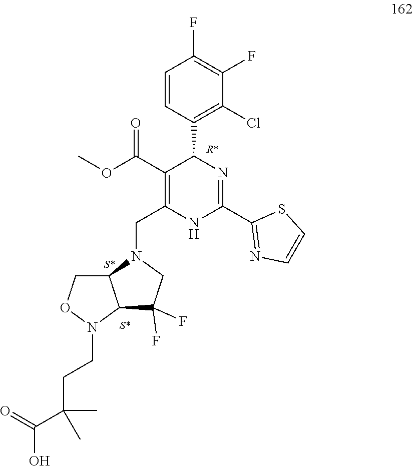 [SMILES: COC(=O)C1=C(CN2CC(F)(F)[C@@H]3[C@H]2CON3CCC(C)(C)C(=O)O)NC(c2nccs2)=N[C@H]1c1ccc(F)c(F)c1Cl]

This compound was made from H5-1A and S37 according to typical coupling method 1 and typical method 2 successively. Purified by Prep. HPLC (Column: Xbridge C8 (5 μm 19*150 mm), Mobile Phase A: water (0.1% trifluoroacetic acid), Mobile Phase B: acetonitrile, UV: 214 nm, Flow rate: 10 mL/min, Gradient: 40-75% (% B)) and C18 column (acetonitril water (0.1% ammonium bicarbonate)=30% to 95%) to give the title compound (33.8 mg, 99.1% purity, 48% yield) as yellow solids. LC-MS (ESI): mass calcd. for C₂₇H₂₈ClF₄N₅O₅S 645.1, m/z found 646.2 [M+H]⁺. ¹H NMR (400 MHz, CDCl₃) δ 9.30 (s, 1H), 7.85 (d, J=2.8 Hz, 1H), 7.44 (d, J=3.2 Hz, 1H), 7.04-7.00 (m, 2H), 6.17 (s, 1H), 4.26-4.22 (m, 3H), 4.06-3.97 (m, 2H), 3.61-3.56 (m, 4H), 3.51-3.40 (m, 1H), 3.07 (t, J=12.4 Hz, 1H), 2.92-2.83 (m, 2H), 2.02-1.84 (m, 2H), 1.27 (s, 3H), 1.25 (s, 3H).

### Compound 163: (cis)-4-(4-((6-(2-chloro-3,4-difluorophenyl)-5-(ethoxycarbonyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

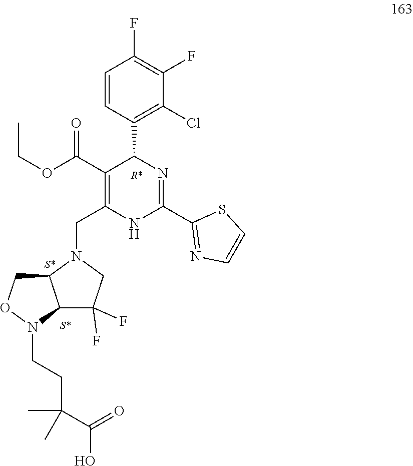 [SMILES: CCOC(=O)C1=C(CN2CC(F)(F)[C@@H]3[C@H]2CON3CCC(C)(C)C(=O)O)NC(c2nccs2)=N[C@H]1c1ccc(F)c(F)c1Cl]

This compound was made from H8-1A and S37 according to typical coupling method 1 and typical method 2 successively. Purified by C18 column (acetonitrile:water (+0.1% ammonium bicarbonateto)=5% to 100%) to give the title compound (44 mg, 98.5% purity, 74% yield) as yellow solids. LC-MS (ESI): mass calcd. for C₂₈H₃₀ClF₄N₅O₅S 659.2, m/z found 600.3 [M+H]⁺. ¹H NMR (400 MHz, CDCl₃) δ 9.27 (s, 1H), 7.85 (d, J=3.2 Hz, 1H), 7.44 (d, J=2.8 Hz, 1H), 7.07-7.00 (m, 2H), 6.19 (s, 1H), 4.26-4.22 (m, 3H), 4.09-3.97 (m, 4H), 3.61-3.56 (m, 1H), 3.51-3.40 (m, 1H), 3.07 (t, J=12.4 Hz, 1H), 2.93-2.82 (m, 2H), 2.02-1.94 (m, 1H), 1.91-1.83 (m, 1H), 1.27 (s, 3H), 1.25 (s, 3H), 1.12 (t, J=7.2 Hz, 3H).

### Compound 164: (cis)-4-(4-((6-(3,4-difluoro-2-methylphenyl)-5-(ethoxycarbonyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

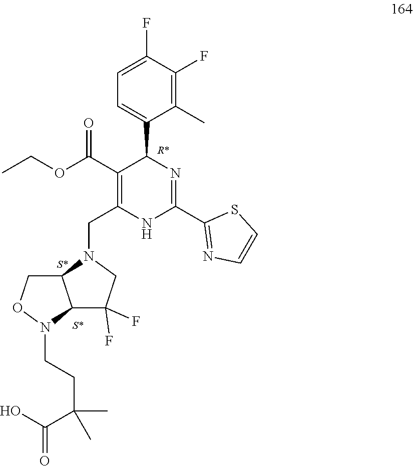 [SMILES: CCOC(=O)C1=C(CN2CC(F)(F)[C@@H]3[C@H]2CON3CCC(C)(C)C(=O)O)NC(c2nccs2)=N[C@@H]1c1ccc(F)c(F)c1C]

This compound was made from H9-1A and S37 according to typical coupling method 1 and typical method 2 successively. Purified with C18 column (acetonitrile:water (5% ammonium bicarbonate)=5% to 100%) to afford the desired product (37 mg, 99% purity, 50% yield) as yellow solids. LC-MS (ESI): mass calcd. for C₂₉H₃₃F₄N₅O₅S 639.2, m/z found 640.3 [M+H]⁺. ¹H NMR (400 MHz, CDCl₃) δ 7.80 (d, J=2.8 Hz, 1H), 7.61 (d, J=2.8 Hz, 1H), 6.95-6.91 (m, 2H), 5.80 (s, 1H), 4.27-4.16 (m, 3H), 3.96-3.90 (m, 4H), 3.76-3.71 (m, 1H), 3.27-3.17 (m, 1H), 3.02 (br s, 1H), 2.83-2.76 (m, 1H), 2.69-2.62 (m, 1H), 2.44 (s, 3H), 1.82-1.65 (m, 2H), 1.10 (s, 6H), 1.03 (t, J=7.2 Hz, 3H).

### Compound 165: (cis)-4-(4-((5-(ethoxycarbonyl)-6-(3-fluoro-2-methylphenyl)-2-(4-methylthiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

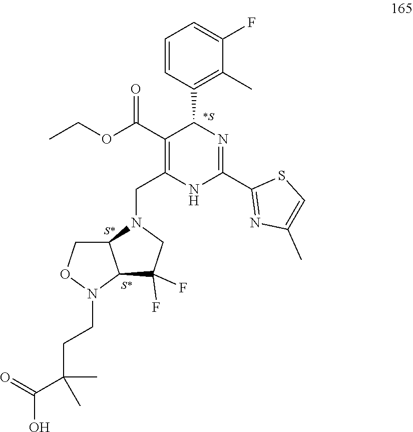 [SMILES: CCOC(=O)C1=C(CN2CC(F)(F)[C@@H]3[C@H]2CON3CCC(C)(C)C(=O)O)NC(c2nc(C)cs2)=N[C@H]1c1cccc(F)c1C]

This compound was made from H22-1B and S37 according to typical coupling method 1 and typical method 2 successively. Purified by C18 column (acetonitrile:water=30% to 80%) to give the title compound (27 mg, 98.5% purity, 51% yield) as yellow solids. LC-MS (ESI): mass calcd. for C₃₀H₃₆F₃N₅O₅S 635.7, m/z found 636.3[M+H]⁺. ¹HNMR (400 MHz, CD₃OD) δ 7.31-7.25 (m, 1H), 7.19-7.09 (m, 2H), 6.97-6.92 (m, 1H), 5.96 (s, 1H), 4.40-4.29 (m, 3H), 4.10-4.00 (m, 4H), 3.88-3.83 (m, 1H), 3.40-3.38 (m, 0.5H), 3.21-3.08 (m, 1.5H), 2.95-2.88 (m, 1H), 2.81-2.75 (m, 1H), 2.51 (s, 3H), 2.47 (s, 3H), 1.95-1.78 (m, 2H), 1.22 (s, 6H), 1.14 (t, J=6.8 Hz, 3H).

### Preparation of Intermediate S40

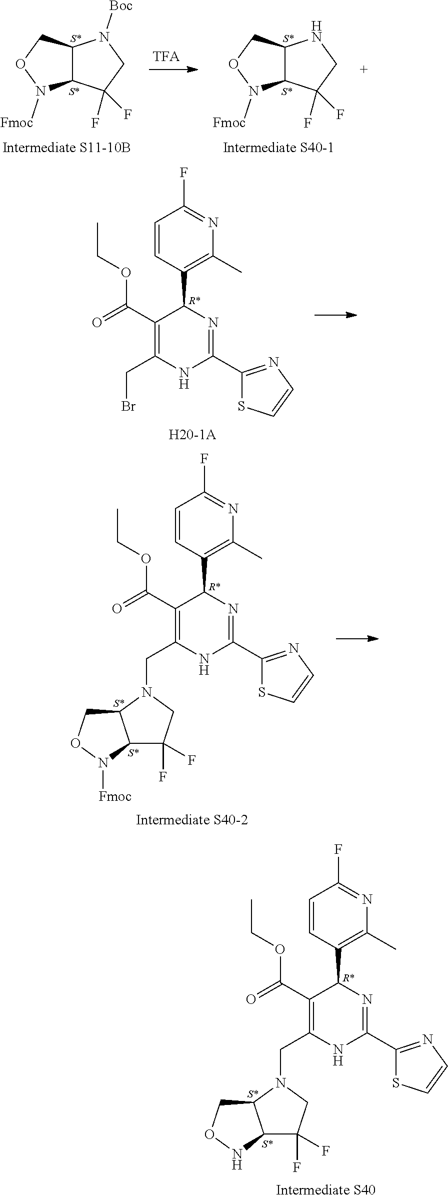

### S40-1: (cis)-(9H-Fluoren-9-yl)methyl 6,6-difluorohexahydro-1H-pyrrolo[3,2-c]isoxazole-1-carboxylate

To a solution of S11-10B (1.90 g, 90% purity, 3.62 mmol) in dichloromethane (8 mL) was added trifluoroacetic acid (4 mL). The reaction mixture was stirred at room temperature for 2 hours. Then it was concentrated and diluted with ethyl acetate (30 mL). The organic solution was washed with sodium bicarbonate solution (10 mL) and brine (10 mL), dried over Na₂SO₄₍ₛ₎, filtered and concentrated to give the title compound (1.30 g, 95% purity by ¹H NMR, 92% yield) as brown solid. ¹H NMR (400 MHz, CDCl₃) δ 7.77 (d, J=7.6 Hz, 2H), 7.63 (t, J=7.2 Hz, 2H), 7.43-7.40 (m, 2H), 7.35-7.30 (m, 2H), 4.58-4.46 (m, 3H), 4.32-4.26 (m, 2H), 4.05-4.03 (m, 1H), 3.63-3.60 (m, 1H), 3.18-3.05 (m, 2H).

### S40-2: (cis)-(9H-Fluoren-9-yl)methyl 4-((5-(ethoxycarbonyl)-6-(6-fluoro-2-methylpyridin-3-yl)-2-(thiazol-2-yl)-3,6-dihydropyrimidin-4-yl)methyl)-6,6-difluorohexahydro-1H-pyrrolo[3,2-c]isoxazole-1-carboxylate

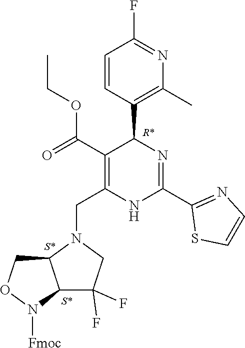

### Intermediate S40-2

This intermediate was made from H20-1A and S40-1 according to typical method 1. LC-MS (ESI): mass calcd. for C₃₇H₃₃F₃N₆O₅S 730.2, m/z found 731.7 [M+H]⁺.

### S40: Ethyl 6-(((cis)-6,6-difluorotetrahydro-1H-pyrrolo[3,2-c]isoxazol-4(5H)-yl)methyl)-4-(6-fluoro-2-methylpyridin-3-yl)-2-(thiazol-2-yl)-1,4-dihydropyrimidine-5-carboxylate

To a solution of S40-2 (160 mg, 100% purity, 0.219 mmol) in N,N-dimethylformamide (2 mL) was added piperidine (80 mg, 0.94 mmol). After stirred at room temperature for 2 hours, ethyl acetate (20 mL) was added. The organic layer was washed with water (10 mL), brine (10 mL), dried over Na₂SO₄₍ₛ₎, filtered and concentrated to give a residue, which was purified by silica gel column chromatography (petroleum ether:ethyl acetate=3:1) to afford the desired product (110 mg, 96% purity), 95% yield as yellow solids. LC-MS (ESI): mass calcd. for C₂₂H₂₃F₃N₆O₃S 508.2, m/z found 509.2 [M+H]⁺.

### Compound 166: 4-((cis)-4-((5-(ethoxycarbonyl)-6-(6-fluoro-2-methylpyridin-3-yl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

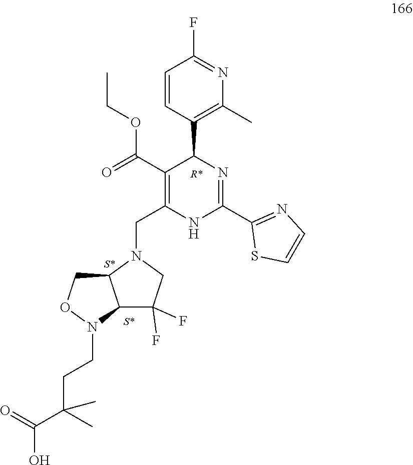 [SMILES: CCOC(=O)C1=C(CN2CC(F)(F)[C@@H]3[C@H]2CON3CCC(C)(C)C(=O)O)NC(c2nccs2)=N[C@@H]1c1ccc(F)nc1C]

This compound was made from S40 and tert-butyl 2,2-dimethyl-4-oxobutanoate using typical method 5 and typical method 3, successively. Purified by C18 column (acetonitrile:water (+0.2% ammonium bicarbonate)=20% to 70%) to afford the desired product (26.1 mg, 98.1% purity, 84% yield) as yellow solids. LC-MS (ESI): mass calcd. for C₂₈H₃₃F₃N₆O₅S 622.2, m/z found 623.3 [M+H]⁺. ¹H NMR (400 MHz, CDCl₃) δ 9.27 (s, 1H), 7.84 (d, J=2.8 Hz, 1H), 7.52 (t, J=8.4 Hz, 1H), 7.44 (d, J=3.2 Hz, 1H), 6.68 (dd, J=8.0, 3.2 Hz, 1H), 5.97 (s, 1H), 4.32-4.20 (m, 3H), 4.09-3.98 (m, 4H), 3.62-3.56 (m, 1H), 3.50-3.41 (m, 1H), 3.10-3.04 (m, 1H), 2.91-2.87 (m, 2H), 2.80 (s, 3H), 2.04-1.95 (m, 1H), 1.91-1.84 (m, 1H), 1.27 (s, 3H), 1.26 (s, 3H), 1.14 (t, J=7.2 Hz, 3H).

### Compound 167: 4-((cis)-4-((6-(2,3-difluorophenyl)-5-(methoxycarbonyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

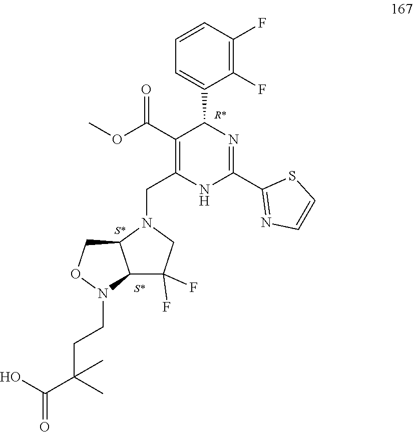 [SMILES: COC(=O)C1=C(CN2CC(F)(F)[C@@H]3[C@H]2CON3CCC(C)(C)C(=O)O)NC(c2nccs2)=N[C@H]1c1cccc(F)c1F]

This compound was made analogous to compound 166 using H23-1A. Purified by C18 (acetonitrile:water (+0.2% ammonium bicarbonate)=10% to 90%) to give the desired compound (30 mg, 95% purity, 52% yield) as yellow solids. LC-MS (ESI): mass calcd. for C₂₇H₂₉F₄N₅O₅S 611.2, m/z found 612.2 [M+H]⁺. ¹H NMR (400 MHz, CDCl₃) δ 9.36 (s, 1H), 7.86 (d, J=3.2 Hz, 1H), 7.45 (d, J=2.8 Hz, 1H) 7.09-7.00 (m, 3H), 6.05 (s, 1H), 4.27-4.20 (m, 2H), 4.14-4.09 (m, 1H), 4.05-3.97 (m, 2H), 3.58 (s, 3H), 3.57-3.55 (m, 1H), 3.52-3.40 (m, 1H), 3.09 (t, J=12.8 Hz, 1H), 2.93-2.82 (m, 2H), 2.00-1.89 (m, 2H), 1.26 (s, 3H), 1.24 (s, 3H).

### Compound 168: 4-((cis)-4-((6-(2-chloro-4-fluorophenyl)-5-(methoxycarbonyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

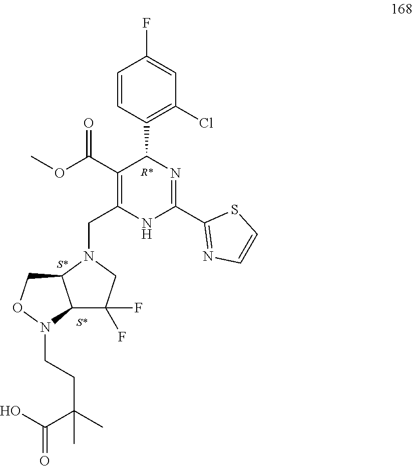 [SMILES: COC(=O)C1=C(CN2CC(F)(F)[C@@H]3[C@H]2CON3CCC(C)(C)C(=O)O)NC(c2nccs2)=N[C@H]1c1ccc(F)cc1Cl]

This compound was made from H3-1A and S37 according to typical coupling method 1 and typical method 2 successively. LC-MS (ESI): mass calcd. for C₂₇H₂₉ClF₃N₅O₅S 627.2, m/z found 628.2 [M+H]⁺. 1H NMR (400 MHz, CDCl3) δ 9.28 (s, 1H), 7.84 (d, J=3.18 Hz, 1H), 7.44 (d, J=3.18 Hz, 1H), 7.27-7.34 (m, 1H), 7.11-7.16 (m, 1H), 6.89-7.00 (m, 1H), 6.17 (s, 1H), 4.19-4.28 (m, 3H), 3.94-4.08 (m, 2H), 3.55-3.64 (m, 4H), 3.34-3.53 (m, 1H), 3.08 (t, J=12.29 Hz, 1H), 2.77-2.96 (m, 2H), 1.80-2.03 (m, 2H), 1.26 (d, J=5.99 Hz, 6H).

### Compound 169: 4-((cis)-4-((6-(2-chloro-4-fluorophenyl)-5-(ethoxycarbonyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid

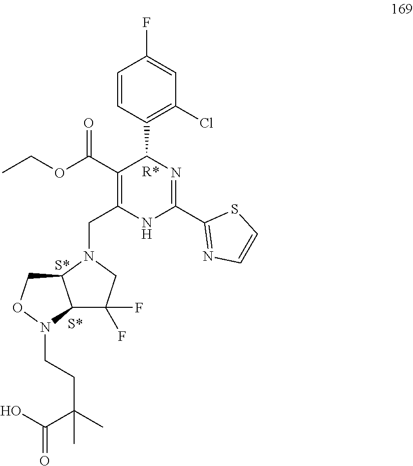 [SMILES: CCOC(=O)C1=C(CN2CC(F)(F)[C@@H]3[C@H]2CON3CCC(C)(C)C(=O)O)NC(c2nccs2)=N[C@H]1c1ccc(F)cc1Cl]

This compound was made from H12-1A and S37 according to typical coupling method 1 and typical method 2 successively. LC-MS (ESI): mass calcd. for C₂₈H₃₁ClF₃N₅O₅S 641.2, m/z found 642.2 [M+H]⁺. ¹H NMR (400 MHz, CDCl3) δ 9.25 (s, 1H), 7.85 (d, J=3.18 Hz, 1H), 7.44 (d, J=3.18 Hz, 1H), 7.27-7.35 (m, 1H), 7.13 (m, 1H), 6.92 (m, 1H), 6.19 (s, 1H), 4.18-4.29 (m, 3H), 3.94-4.08 (m, 4H), 3.60 (m, 1H), 3.34-3.53 (m, 1H), 3.08 (br t, J=12.29 Hz, 1H), 2.76-2.97 (m, 2H), 1.78-2.02 (m, 2H), 1.26 (m, 6H), 1.13 (t, J=7.15 Hz, 3H).

### Compound 170: (cis)-4-(4-((6-(2-chloro-3-fluorophenyl)-5-(methoxycarbonyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

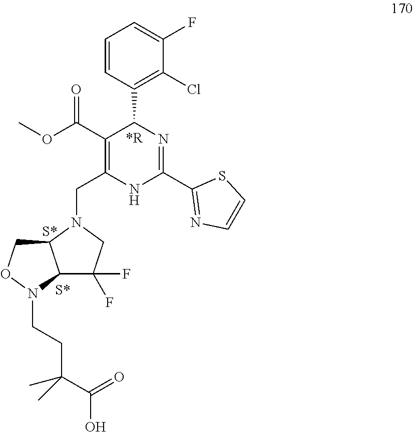 [SMILES: COC(=O)C1=C(CN2CC(F)(F)[C@@H]3[C@H]2CON3CCC(C)(C)C(=O)O)NC(c2nccs2)=N[C@H]1c1cccc(F)c1Cl]

This compound was made from H11-1A and S37 according to typical coupling method 1 and typical method 2 successively. LC-MS (ESI): mass calcd. for C₂₇H₂₉ClF₃N₅O₅S 627.2, m/z found 628.2 [M+H]⁺. ¹H NMR (400 MHz, METHANOL-d4) δ ppm 7.91 (1H, d, J=3.18 Hz), 7.73 (1H, d, J=2.93 Hz), 7.19-7.33 (2H, m), 7.11-7.18 (1H, m), 6.19 (1H, s), 4.15-4.40 (3H, m), 4.00 (2H, br d, J=2.93 Hz), 3.73-3.93 (1H, m), 3.58 (3H, s), 3.31-3.38 (1H, m), 3.05-3.20 (1H, m), 2.89 (1H, td, J=11.46, 4.83 Hz), 2.75 (1H, td, J=11.43, 5.50 Hz), 1.74-1.94 (2H, m), 1.20 (6H, s).

### Compound 171: (cis)-4-(4-((6-(2-chloro-3-fluorophenyl)-5-(ethoxycarbonyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

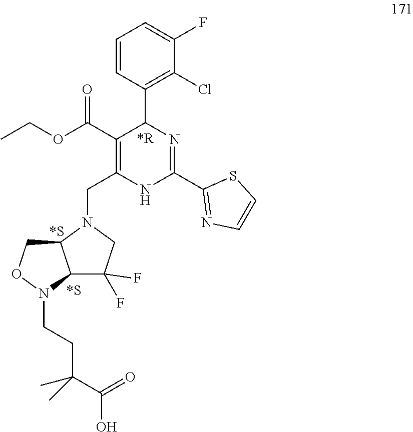 [SMILES: CCOC(=O)C1=C(CN2CC(F)(F)[C@@H]3[C@H]2CON3CCC(C)(C)C(=O)O)NC(c2nccs2)=NC1c1cccc(F)c1Cl]

This compound was made from H1-1A and S37 according to typical coupling method 1 and typical method 2 successively. LC-MS (ESI): mass calcd. for C₂₇H₃₁ClF₃N₅O₅S 641.2, m/z found 642.2 [M+H]⁺. 1H NMR (400 MHz, METHANOL-d4) δ ppm 7.91 (1H, d, J=2.93 Hz), 7.69-7.77 (1H, m), 7.20-7.38 (2H, m), 7.10-7.20 (1H, m), 6.21 (1H, s), 4.16-4.40 (3H, m), 3.94-4.09 (4H, m), 3.77-3.88 (1H, m), 3.32-3.39 (1H, m), 3.07-3.20 (1H, m), 2.89 (1H, td, J=11.40, 4.58 Hz), 2.76 (1H, dt, J=11.13, 5.69 Hz), 1.74-1.95 (2H, m), 1.20 (6H, s), 1.10 (3H, t, J=7.03 Hz).

### Compound 172: 3-((cis)-4-((5-(Ethoxycarbonyl)-6-(6-fluoro-2-methylpyridin-3-yl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylpropanoic acid (Single Diastereomer)

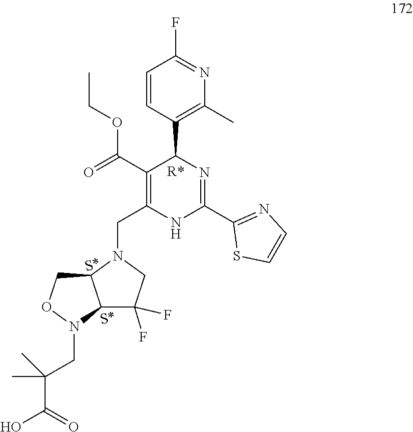 [SMILES: CCOC(=O)C1=C(CN2CC(F)(F)[C@@H]3[C@H]2CON3CC(C)(C)C(=O)O)NC(c2nccs2)=N[C@@H]1c1ccc(F)nc1C]

This compound was made analogous to compound 166 using tert-butyl 2,2-dimethyl-3-oxopropanoate. purified by C18 column (acetonitrile:water (+0.2% ammonium bicarbonate)=20% to 70%) to afford the desired product (24.5 mg, 98.4% purity, 59.7% yield) as yellow solids. LC-MS (ESI): mass calcd. for C₂H₃₁F₃N₆O₅S 608.6, m/z found 609.3 [M+H]⁺. ¹H NMR (400 MHz, CDCl₃) δ 9.26 (s, 1H), 7.85 (d, J=3.2 Hz, 1H), 7.52 (t, J=8.4 Hz, 1H), 7.44 (d, J=3.2 Hz, 1H), 6.68 (dd, J=8.4 Hz, 3.2 Hz, 1H), 5.97 (s, 1H), 4.30-4.20 (m, 3H), 4.10-3.99 (m, 4H), 3.70-3.65 (m, 1H), 3.46-3.36 (m, 1H), 3.22-3.18 (m, 1H), 3.10-3.04 (m, 1H), 2.80-2.77 (m, 4H), 1.31 (s, 3H), 1.29 (s, 3H), 1.13 (t, J=7.2 Hz, 3H).

### Compound 173: 4-((cis)-4-((6-(2,3-difluorophenyl)-5-(ethoxycarbonyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

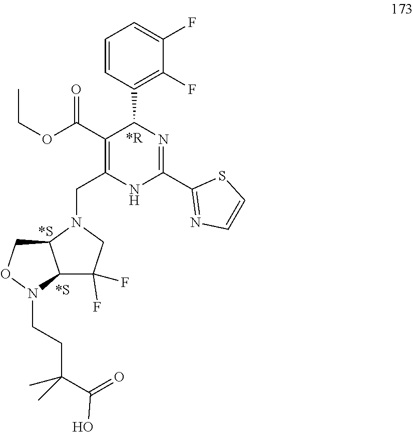 [SMILES: CCOC(=O)C1=C(CN2CC(F)(F)[C@@H]3[C@H]2CON3CCC(C)(C)C(=O)O)NC(c2nccs2)=N[C@H]1c1cccc(F)c1F]

This compound was made from H24-1A and S37 according to typical coupling method 1 and typical method 2 successively. Purified by C18 column (acetonitrile:water (0.1% ammonium bicarbonate=15% to 70%) to give the title compound (60 mg, 99.4% purity, 88% yield) as yellow solids. LC-MS (ESI): mass calcd. for C₂₈H₃₁F₄N₅O₅S 625.2, m/z found 626.3 [M+H]⁺. ¹H NMR (400 MHz, CDCl₃) δ 9.33 (s, 1H), 7.86 (d, J=3.2 Hz, 1H), 7.44 (d, J=2.8 Hz, 1H), 7.10-6.99 (m, 3H), 6.05 (s, 1H), 4.27-4.20 (m, 2H), 4.13-3.97 (m, 5H), 3.63-3.58 (m, 1H), 3.46-3.31 (m, 1H), 3.07 (t, J=12.0 Hz, 1H), 2.92-2.80 (m, 2H), 1.96-1.78 (m, 2H), 1.23 (s, 3H), 1.22 (s, 3H), 1.16 (t, J=6.8 Hz, 3H).

### Compound 174: 4-((cis)-4-((5-(ethoxycarbonyl)-2-(4-methylthiazol-2-yl)-6-(2,3,4-trifluorophenyl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

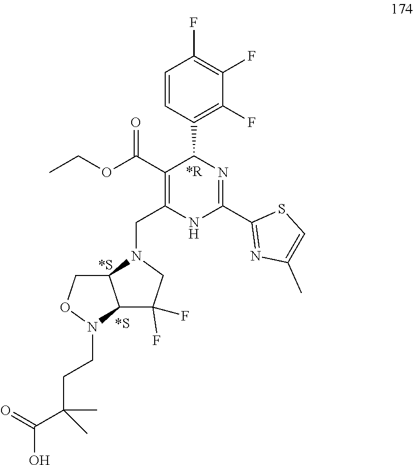 [SMILES: CCOC(=O)C1=C(CN2CC(F)(F)[C@@H]3[C@H]2CON3CCC(C)(C)C(=O)O)NC(c2nc(C)cs2)=N[C@H]1c1ccc(F)c(F)c1F]

This compound was made from H27-1A and S37 according to typical coupling method 1 and typical method 2 successively. LC-MS (ESI): mass calcd. for C₂₉H₃₂F₅N₅O₅S 657.2, m/z found 658.3 [M+H]⁺. ¹H NMR (400 MHz, CDCl₃) δ 9.32 (s, 1H), 7.05-6.99 (m, 2H), 6.91-6.85 (m, 1H), 5.99 (s, 1H), 4.24-3.96 (m, 7H), 3.57 (dd, J=14.0, 7.2 Hz, 1H), 3.49-3.38 (m, 1H), 3.09 (t, J=12.0 Hz, 1H), 2.90-2.84 (m, 2H), 2.47 (s, 3H), 1.96-1.89 (m, 2H), 1.26 (d, J=4.0 Hz, 6H), 1.18 (t, J=7.2 Hz, 3H).

### Compound 175: 4-((cis)-4-((5-(Ethoxycarbonyl)-2-(thiazol-2-yl)-6-(2,3,4-trifluorophenyl)-3,6-dihydropyrimidin-4-yl)methyl)-6,6-difluorohexahydro-1H-pyrrolo[3,2-c]isoxazol-1-yl)-2,2-dimethylbutanoic acid (Single Diastereomer)

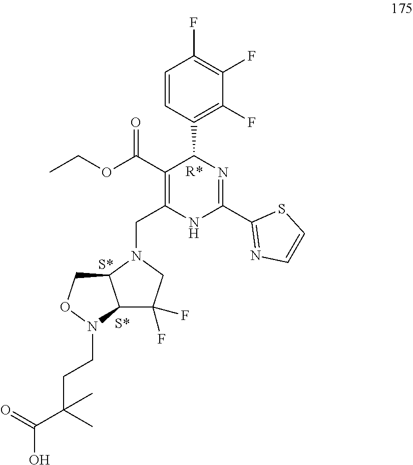 [SMILES: CCOC(=O)C1=C(CN2CC(F)(F)[C@@H]3[C@H]2CON3CCC(C)(C)C(=O)O)NC(c2nccs2)=N[C@H]1c1ccc(F)c(F)c1F]

This compound was made from H25-1A and S37 according to typical coupling method 1 and typical method 2 successively. LC-MS (ESI): mass calcd. for C₂₈H₃₀F₅N₅O₅S 643.6, m/z found 644.2 [M+H]⁺. ¹H NMR (400 MHz, CDCl₃) δ 9.34 (s, 1H), 7.87 (d, J=3.2 Hz, 1H), 7.46 (d, J=3.2 Hz, 1H), 7.06-7.00 (m, 1H), 6.93-6.86 (m, 1H), 6.00 (s, 1H), 4.26-4.19 (m, 2H), 4.14-3.96 (m, 5H), 3.60-3.55 (m, 1H), 3.50-3.39 (m, 1H), 3.10-3.04 (m, 1H), 2.93-2.80 (m, 2H), 2.00-1.84 (m, 2H), 1.26 (s, 3H), 1.24 (s, 3H), 1.19-1.16 (m, 3H).

### Compound 176: 3-((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydro-1H-pyrrolo[3,2-c]isoxazol-1-yl)-2,2-dimethylpropanoic acid (Single Diastereomer)

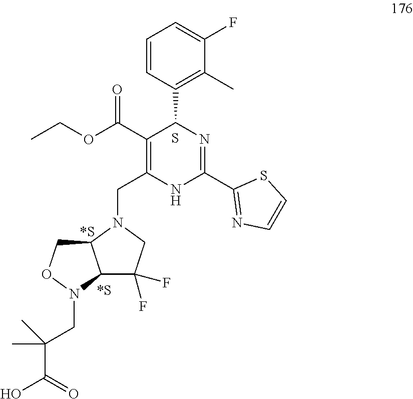

This compound was made analogous to compound 166 from H2-1A, S11-10B and tert-butyl 2,2-dimethyl-3-oxopropanoate. Purified by Prep. HPLC (Column: Waters Xbrige C18 (5 μm 19*150 mm), Mobile phase A: water (0.1% ammonium hydrogencarbonat), Mobile phase B: acetonitrile, UV: 214 nm, Flow rate: 15 mL/min, Gradient: 30-80% (% B)) to give the desired product (17 mg, 98% purity, 24% yield) as yellow solids. LC-MS (ESI): mass calcd. for C₂₈H₃₂F₃N₅O₅S 607.2, m/z found 608.2 [M+H]⁺. ¹H NMR (400 MHz, CDCl₃) δ 9.17 (s, 1H), 7.82 (d, J=3.2 Hz, 1H), 7.41 (d, J=3.2 Hz, 1H), 7.10-7.04 (m, 1H), 6.97-6.88 (m, 2H), 6.00 (s, 1H), 4.32-4.20 (m, 3H), 4.10-3.97 (m, 4H), 3.71-3.66 (m, 1H), 3.47-3.36 (m, 1H), 3.20-3.17 (m, 1H), 3.12-3.06 (m, 1H), 2.82-2.79 (m, 1H), 2.54 (s, 3H), 1.30 (s, 6H), 1.11 (t, J=7.2 Hz, 3H).

### Preparation of Intermediate S76

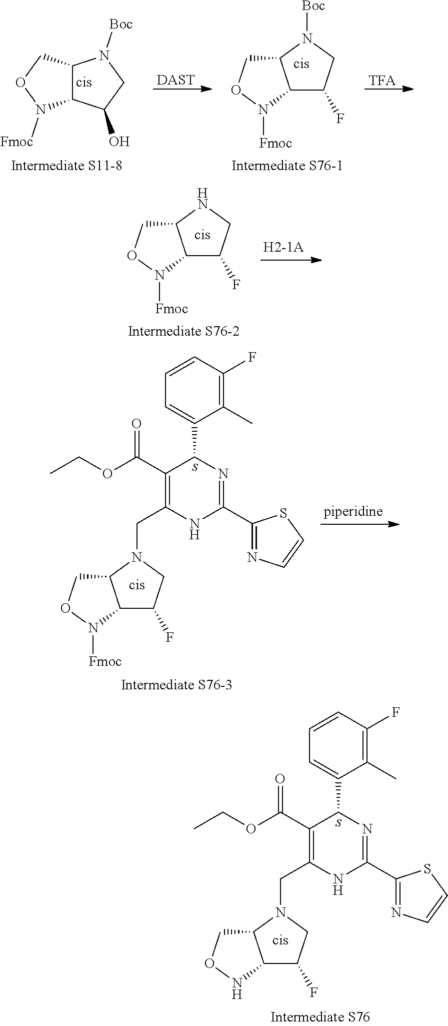

### S76-1: cis-1-((9H-fluoren-9-yl)methyl) 4-tert-butyl 6-fluorotetrahydro-1H-pyrrolo[3,2-c]isoxazole-1,4(5H)-dicarboxylate

To a solution of S11-8 (3.0 g, 6.37 mmol, 96% purity) in toluene (30 mL) was added diethylaminosulfur trifluoride (7.2 g, 44.6 mmol). The mixture was refluxed overnight under nitrogen. Then it was poured into water (20 mL) and sodium bicarbonate (10 g) was added. The mixture was extracted with dichloromethane (50 mL) for three times. The organic layers were dried over sodium sulfate and concentrate under vacuum to give a crude, which was purified by column chromatography on silica gel (petroleum ether:ethyl acetate=10:1) to give the title product (1.1 g, 99% purity by ¹H NMR, 38% yield) as orange oil. ¹H NMR (400 MHz, CDCl₃) δ 7.79-7.77 (m, 2H), 7.62-7.59 (m, 2H), 7.44-7.42 (m, 2H), 7.40-7.32 (m, 2H), 5.01-5.00 (m, 1H), 4.89-4.88 (m, 1H), 4.80-4.70 (m, 2H), 4.50-4.23 (m, 1H), 4.29-4.11 (m, 2H), 4.00-3.85 (m, 1H), 3.58-3.50 (m, 2H), 1.48 (s, 9H).

### S76-2: cis-(9H-fluoren-9-yl)methyl 6-fluorohexahydro-1H-pyrrolo[3,2-c]isoxazole-1-carboxylate trifluoroacetate

A mixture of S76-1 (1.2 g, 2.61 mmol, 99% purity) and 2,2,2-trifluoroacetic acid (15 mL) in dichloromethane (45 mL) was stirred at room temperature for 1 hour. The mixture was concentrated under vacuum to give the title product (1.1 g, 95% purity from ¹H NMR, 85% yield) as brown solids. ¹H NMR (400 MHz, CDCl₃) δ 7.79-7.77 (m, 2H), 7.62-7.59 (m, 2H), 7.44-7.42 (m, 2H), 7.40-7.32 (m, 2H), 5.01-5.00 (m, 1H), 4.89-4.88 (m, 1H), 4.80-4.70 (m, 2H), 4.50-4.23 (m, 1H), 4.29-4.11 (m, 2H), 4.00-3.85 (m, 1H), 3.58-3.50 (m, 2H).

### S76-3: cis-(9H-fluoren-9-yl)methyl 4-(((S)-5-(ethoxycarbonyl)-6-(3-fluoro-2-methylphenyl)-2-(thiazol-2-yl)-3,6-dihydropyrimidin-4-yl)methyl)-6-fluorohexahydro-1H-pyrrolo[3,2-c]isoxazole-1-carboxylate

To a solution of S76-2 (1.1 g, 2.95 mmol, 95% purity) in N,N-dimethylformamide (5 mL) was added nitrilotrimethanol (2.64 g, 17.7 mmol) and h2-1A (1.32 g, 3.01 mmol, 95% purity). The mixture was stirred at 40° C. overnight. Then it was poured into water (20 mL) and extracted with ethyl acetate (20 mL) for three times. The organic layers were washed with brine (20 mL) and dried over sodium sulfate. The mixture was concentrated under vacuum to give a crude, which was purified by column chromatography on silica gel (petroleum ether:ethyl acetate=2:1) to give the title product (700 mg, 66% purity, 22% yield) as yellow solids. LC-MS (ESI): mass calcd. for C₃₈H₃₅F₂N₅O₅S 711.2, m/z found 712.7 [M+H]⁺. ¹H NMR (400 MHz, CDCl₃) δ 9.39-9.33 (m, 1H) 7.89 (d, J=4.4 Hz, 1H), 7.82 (d, J=9.6 Hz, 2H), 7.65-7.70 (m, 2H), 7.48-7.44 (m, 2H), 7.41-7.35 (m, 2H), 7.13-7.04 (m, 2H), 7.02-6.93 (m, 2H), 6.75 (d, J=7.2 Hz, 1H), 5.24 (s, 0.5H), 5.07 (s, 0.5H), 4.91-4.81 (m, 1H), 4.70-4.62 (m, 1H), 4.57-4.58 (m, 2H), 4.34-4.31 (m, 2H), 4.28-4.21 (m, 1H), 4.15-4.06 (m, 3H), 3.58-3.45 (m, 1H), 3.40-3.11 (m, 2H), 2.60 (s, 3H), 1.21-1.16 (m, 3H).

### S76: cis-(S)-4-(3-fluoro-2-methylphenyl)-6-((6-fluorotetrahydro-1H-pyrrolo[3,2-c]isoxazol-4(5H)-yl)methyl)-2-(thiazol-2-yl)-1,4-dihydropyrimidine-5-carboxylate

To a solution of S76-3 (675 mg, 0.939 mmol) in N,N-dimethylformamide (10 mL) was added piperidine (336 mg, 3.94 mmol) and the mixture was stirred at room temperature for 3 hours. Then it was directly purified by C18 column (acetonitrile:water=10% to 100%) to give the desired compound (400 mg, 84% yield, 97% purity) as yellow solids. LC-MS (ESI): mass calcd. for C₂₃H₂₅F₂N₅O₃S 489.2, m/z found 490.3 [M+H]⁺. ¹H NMR (400 MHz, CDCl₃) δ 9.40 (s, 1H) 7.82 (d, J=3.2 Hz, 1H), 7.40 (d, J=3.2 Hz, 1H), 7.09-7.05 (m, 1H), 6.99 (d, J=3.2 Hz, 1H), 6.90 (t, J=4.2 Hz, 1H), 6.02 (s, 1H), 5.30 (s, 1H), 5.18 (s, 0.5H), 5.05 (s, 0.5H), 4.41 (d, J=17.3 Hz, 1H), 4.33 (d, J=10 Hz, 1H), 4.30-4.23 (m, 1H), 4.15 (m, 1H), 4.08-4.0 (m, 2H), 3.38-3.34 (m, 1H), 3.28 (dd, J=3.6, 12 Hz, 0.5H), 3.20 (dd, J=3.6, 12 Hz, 0.5H), 3.09-3.01 (m, 1H), 2.54 (d, J=1.6 Hz, 3H), 1.12 (t, J=7.2 Hz, 3H).

### Compound 177: 4-((cis)-4-(((S)-5-(ethoxycarbonyl)-6-(3-fluoro-2-methylphenyl)-2-(thiazol-2-yl)-3,6-dihydropyrimidin-4-yl)methyl)-6-fluorohexahydro-1H-pyrrolo[3,2-c]isoxazol-1-yl)-2,2-dimethylbutanoic acid

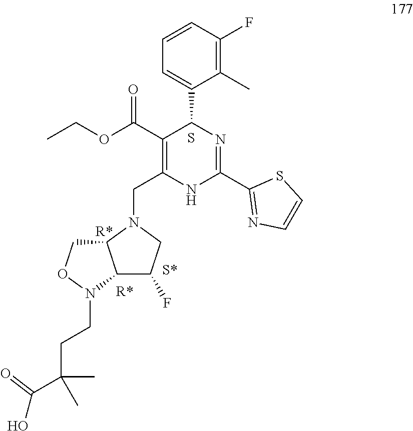

This compound was made from S76 and tert-butyl 2,2-dimethyl-4-oxobutanoate according to typical method 5 and 3 successively. Chiral separation of tert-butyl ester compounds: Column: Chiralpak OD-H 5 μm 20*250 mm; Mobile Phase: Hex:EtOH=95:5 at 15 mL/min; Temp: 30° C.; Wavelength: 254 nm.

Compound 177: LC-MS (ESI): mass calcd. for C₂₉H₃₅F₂N₅O₅S 603.2, m/z found 604.3 [M+H]⁺. ¹H NMR (400 MHz, CDCl₃) δ 9.33 (s, 1H), 7.81 (d, J=3.2 Hz, 1H), 7.39 (d, J=3.2 Hz, 1H), 7.09-7.04 (m, 1H), 6.99-6.97 (t, 1H), 6.92-6.88 (m, 1H), 6.00 (s, 1H), 5.1 (s, 0.5H), 5.0 (s, 0.5H), 4.24-4.21 (m, 3H), 3.99-3.94 (m, 3H), 3.90-3.87 (m, 1H), 3.77-3.70 (m, 1H), 3.34-3.25 (m, 1H), 3.08-3.01 (m, 1H), 2.93-2.90 (m, 1H), 2.82-2.77 (m, 2H), 2.54 (s, 3H), 1.97-1.81 (m, 2H), 1.26 (s, 3H), 1.24 (s, 3H), 1.13-1.09 (t, J=2.8 Hz, 3H).

### Preparation of Intermediate S75

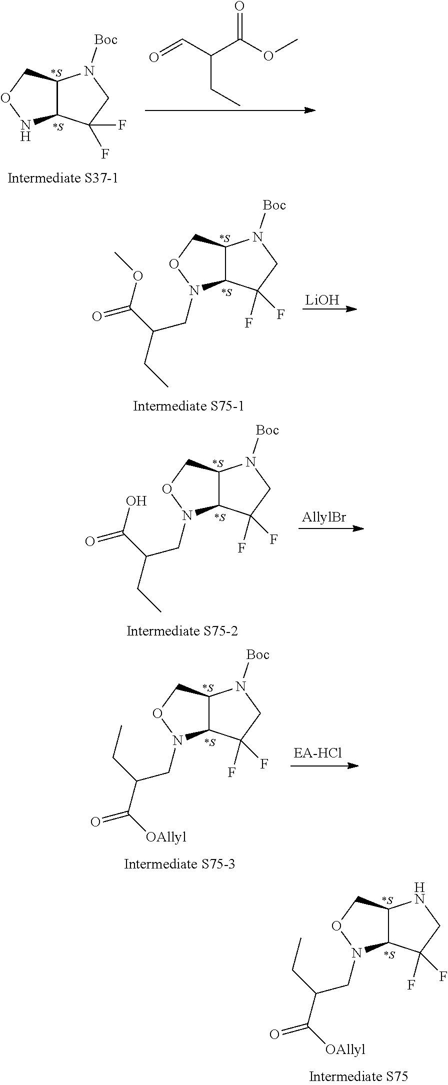

### S75-1: (cis)-tert-butyl 6,6-difluoro-1-(2-(methoxycarbonyl)butyl)tetrahydro-1H-pyrrolo[3,2-c]isoxazole-4(5H)-carboxylate

To a solution of S37-1 (500 mg, 1.80 mmol, 90% purity) in methanol (15 mL) was added methyl 2-formylbutanoate (1.56 g, 3.60 mmol, 30% purity) and acetic acid (5 mL). The mixture was stirred at room temperature for 1 hour, then sodium triacetoxyhydroborate (1.89 g, 5.36 mmol) was added and stirred overnight. The mixture was quenched with saturated sodium carbonate aqueous solution (30 mL) and extracted with ethyl acetate (30 mL) for three times. The combined organic layers were washed with brine (30 mL) and dried over anhydrous sodium sulfate (s). The mixture was filtered and the filtrate was concentrated to give a crude, which was purified by chromatography column on silica gel (petroleum ether:ethyl acetate=8:1) to give the desired compound (520 mg, 71% yield, 90% purity from ¹H NMR) as pale yellow oil. ¹H NMR (400 MHz, CDCl₃) δ 4.85-4.75 (m, 1H), 4.24-4.13 (m, 3H), 3.95-3.58 (m, 4H), 3.45-2.89 (m, 3H), 2.35-2.17 (m, 1H), 1.74-1.65 (m, 2H), 1.45 (s, 9H), 0.95-0.90 (m, 3H).

### S75-2: 2-(((cis)-4-(tert-butoxycarbonyl)-6,6-difluorohexahydro-1H-pyrrolo[3,2-c]isoxazol-1-yl)methyl)butanoic acid

To a solution of S75-1 (520 mg, 1.28 mmol, 90% purity) in tetrahydrofuran (5 mL) was added methanol (2.5 mL), lithium hydroxide monohydrate (270 mg, 6.43 mmol) and water (2.5 mL). The mixture was stirred at 30° C. overnight. The mixture was poured into saturated sodium carbonate aqueous solution (30 mL) and extracted with ethyl acetate (30 mL) for three times. The combined organic layers were washed with brine (30 mL) and dried over anhydrous sodium sulfate (s). The mixture was filtered and the filtrate was concentrated to give the title compound (460 mg, 92% yield, 19% purity) as yellow oil. LC-MS (ESI): mass calcd. for C₁₅H₂₄F₂N₂O₅ 350.2, m/z found 351.3 [M+H]⁺.

### S75-3: (cis)-tert-butyl 1-(2-((allyloxy)carbonyl)butyl)-6,6-difluorotetrahydro-1H-pyrrolo[3,2-c]isoxazole-4(5H)-carboxylate

To a mixture of S75-2 (460 mg, 1.18 mmol, 90% purity) and potassium carbonate (490 mg, 3.55 mmol) in N,N-dimethylformamide (3 mL) was added 3-bromoprop-1-ene (172 mg, 1.42 mmol). The mixture was stirred at room temperature for 3 hours. The reaction mixture was poured into water (30 mL) and extracted with ethyl acetate (30 mL) for three times. The combined organic phases were washed with brine (30 mL) and dried over anhydrous sodium sulfate (s). The mixture was filtered and the filtrate was concentrated under reduced pressure to give the crude product, which was purified by column chromatography on silica gel (petroleum ether:ethyl acetate=5:1) to give the desired product (350 mg, 68% yield, 90% purity by ¹H NMR) as yellow oil. ¹H NMR (400 MHz, CDCl₃) δ 5.97-5.87 (m, 1H), 5.35-5.22 (m, 2H), 4.85-4.73 (m, 1H), 4.64-4.55 (m, 2H), 4.12-4.03 (m, 1H), 3.95-3.84 (m, 2H), 3.68-3.60 (m, 1H), 3.46-3.39 (m, 1H), 3.25-3.10 (m, 1H), 3.00-2.82 (m, 1H), 2.79-2.70 (m, 1H), 1.77-1.65 (m, 2H), 1.45 (s, 9H), 0.96-0.91 (m, 3H).

### S75: Allyl 2-(((cis)-6,6-difluorohexahydro-1H-pyrrolo[3,2-c]isoxazol-1-yl)methyl)butanoate

To a solution of S75-3 (350 mg, 0.807 mmol, 90% purity) in ethyl acetate (15 mL) was added 4 M hydrochloride in ethyl acetate (5 mL). The mixture was stirred at 0° C. for 8 hours. The mixture was washed with saturated sodium bicarbonate aqueous solution (10 mL) and extracted with dichloromethane (10 mL) for three times. The combined organic layers were washed with brine (30 mL) and dried over anhydrous sodium sulfate (s). The mixture was filtered and the filtrate was concentrated to give the title compound (220 mg, 94% yield, 100% purity) as pale yellow oil. LC-MS (ESI): mass calcd. for C₁₃H₂₀F₂N₂O₃ 290.1, m/z found 291.2 [M+H]⁺.

### Compound 178A and 178B: 2-(((cis)-4-(((S)-5-(ethoxycarbonyl)-6-(3-fluoro-2-methylphenyl)-2-(thiazol-2-yl)-3,6-dihydropyrimidin-4-yl)methyl)-6,6-difluorohexahydro-1H-pyrrolo[3,2-c]isoxazol-1-yl)methyl)butanoic acid

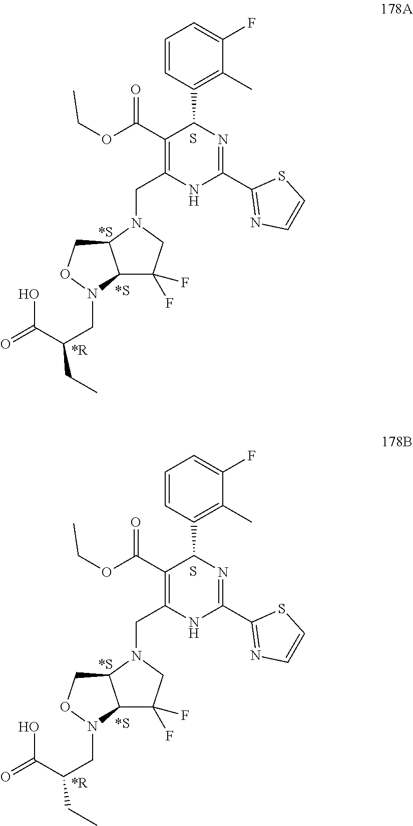

These two compounds were made from H2-1A and S75 according to typical method 1 and 2 successively. Chiral separation of allyl ester compounds: Column: Chiralpak IE 5 μm 20*250 mm; Mobile Phase: Hex:EtOH=90:10 at 20 mL/min; Temp: 30° C.; Wavelength: 254 nm.

178A: LC-MS (ESI): mass calcd. for C₂₈H₃₂F₃N₅O₅S 607.2, m/z found 608.2 [M+H]⁺. ¹H NMR (400 MHz, CDCl₃) δ 9.17 (s, 1H), 7.81 (d, J=3.2 Hz, 1H), 7.40 (d, J=3.2 Hz, 1H), 7.10-7.04 (m, 1H), 6.97-6.95 (m, 1H), 6.93-6.88 (m, 1H), 6.00 (s, 1H), 4.31-4.20 (m, 3H), 4.08-3.97 (m, 4H), 3.67-3.61 (m, 1H), 3.45-3.35 (m, 1H), 3.24-3.19 (m, 1H), 3.11-3.05 (m, 1H), 2.85-2.80 (m, 1H), 2.75-2.70 (m, 1H), 2.54 (s, 3H), 1.78-1.70 (m, 2H), 1.11 (t, J=7.2 Hz, 3H), 0.98 (t, J=7.2 Hz, 3H).

178B: LC-MS (ESI): mass calcd. for C₂₈H₃₂F₃N₅O₅S 607.2, m/z found 608.3 [M+H]⁺. ¹H NMR (400 MHz, CDCl₃) δ 9.17 (s, 1H), 7.83 (d, J=3.2 Hz, 1H), 7.40 (d, J=2.8 Hz, 1H), 7.09-7.04 (m, 1H), 6.97-6.95 (m, 1H), 6.93-6.88 (m, 1H), 6.00 (s, 1H), 4.31-4.22 (m, 3H), 4.10-3.97 (m, 4H), 3.75-3.65 (m, 1H), 3.47-3.38 (m, 1H), 3.14-3.08 (m, 1H), 3.04-3.02 (m, 2H), 2.73-2.70 (m, 1H), 2.54 (s, 3H), 1.79-1.70 (m, 1H), 1.64-1.55 (m, 1H), 1.11 (t, J=7.2 Hz, 3H), 0.97 (t, J=7.2 Hz, 3H).

### Preparation of Intermediate T4

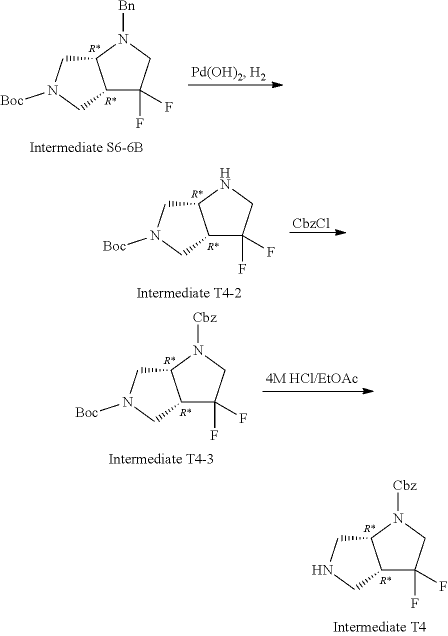 [SMILES: CC(C)(C)OC(=O)N1C[C@@H]2[C@H](C1)N(Cc1ccccc1)CC2(F)F ; "CbzCl" ; CC(C)(C)OC(=O)N1C[C@@H]2NCC(F)(F)[C@@H]2C1 ; CC(C)(C)OC(=O)N1C[C@@H]2[C@H](C1)N(C(=O)OCc1ccccc1)CC2(F)F ; O=C(OCc1ccccc1)N1CC(F)(F)[C@@H]2CNC[C@@H]21]

### T4-2: (cis)-tert-Butyl 3,3-difluorohexahydropyrrolo[3,4-b]pyrrole-5(1H)-carboxylate

To a solution of S6-6B (43.0 g, 90% purity, 114 mmol) in isopropyl alcohol (200 mL) was added 20% wt. palladium hydroxide on charcoal (11.0 g, 15.7 mmol). The reaction mixture was stirred under hydrogen atmosphere (50 psi) overnight at 50° C. Then it was filtered, and the cake was washed with isopropyl alcohol (50 mL) twice. The filtrate was concentrated in vacuo to give the title compound (30.0 g, 90% purity from ¹H NMR, 95% yield) as white solids. LC-MS (ESI): mass calcd. for C₁₁H₁₈F₂N₂O₂ 248.1, m/z found 193.1[M−56+H]⁺. ¹H NMR (400 MHz, CDCl₃) δ 4.08-4.04 (m, 1H), 3.72-3.68 (m, 1H), 3.52-3.44 (m, 3H), 3.27-3.20 (m, 2H), 2.93-2.86 (m, 1H), 1.46 (s, 9H).

### T4-3: (cis)-1-Benzyl 5-tert-butyl 3,3-difluorohexahydropyrrolo[3,4-b]pyrrole-1,5-dicarboxylate

To a solution of T4-2 (30.0 g, 90% purity, 108 mmol) in tetrahydrofuran (200 mL) was added benzyl carbonochloridate (22 mL, 154 mmol) and a solution of sodium bicarbonate (11.0 g, 131 mmol) in water (40 mL) at room temperature. After stirred at room temperature overnight, the mixture was poured into water (1000 mL) and extracted with ethyl acetate (500 mL) for three times. The combined organic layers were washed with brine (500 mL), dried over Na₂SO₄₍ₛ₎, filtered and concentrated in vacuo to give a residue, which was purified by silica gel column chromatography (petroleum ether:ethyl acetate=20:1 to 10:1) and C18 column (acetonitrile:water=5% to 95%) to give the title compound (45.0 g, 90% purity from ¹H NMR, 95% yield) as white solids. LC-MS (ESI): mass calcd. for C₁₉H₂₄F₂N₂O₄ 382.2, m/z found 383.1 [M+H]⁺. ¹H NMR (400 MHz, CDCl₃) δ 7.39-7.33 (m, 5H), 5.14 (br s, 2H), 4.53-4.49 (m, 1H), 4.01-3.91 (m, 1H), 3.79-3.50 (m, 5H), 3.13-3.11 (m, 1H), 1.45 (s, 9H).

### T4: (cis)-Benzyl 3,3-difluorohexahydropyrrolo[3,4-b]pyrrole-1(2H)-carboxylate hydrochloride

To a solution of T4-3 (1.2 g, 96% purity, 3.01 mmol) in ethyl acetate (5 mL) was added 4.0 M hydrochloride in ethyl acetate (5 mL) under nitrogen atmosphere. After stirred at room temperature for 2 hours, the mixture was concentrated to give the title compound (1.0 g, 90% purity from ¹H NMR, 94% yield) as white solids. LC-MS (ESI): mass calcd. for C₁₄H₁₆F₂N₂O₂ 282.3, m/z found 283.1 [M+H]⁺. ¹H NMR (400 MHz, CDCl₃) δ 7.41-7.35 (m, 5H), 5.23-5.11 (m, 2H), 4.60-4.44 (m, 1H), 4.08-3.94 (m, 1H), 3.63-3.52 (m, 1H), 3.37-3.19 (m, 2H), 3.00-2.87 (m, 3H).

### Compound 179-A: tert-butyl (cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrole-5(1H)-carboxylate

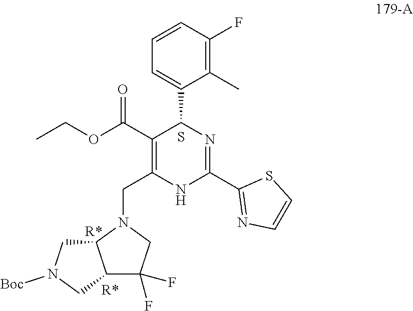

This compound was made from H2-1A and T4-2 according to typical method 1. LC-MS (ESI): mass calcd. for C₂₉H₃₄F₃N₅O₄S 605.2, m/z found 606.6 [M+H]⁺.

### Compound 179: ethyl (S)-6-(((cis)-3,3-difluorohexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate

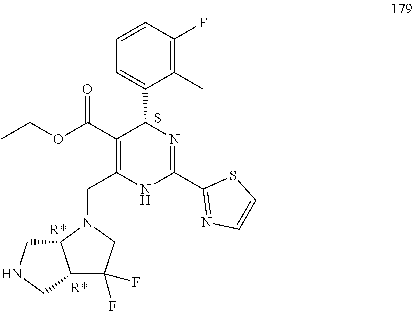

A solution of compound 179-A (1.4 g, 39% purity, 0.901 mmol) in 4 M hydrochloride in 1,4-dioxane solution (35 mL) was stirred for 1 hour at room temperature. Then the mixture was concentrated under reduced pressure to give a residue, which was diluted with ethyl acetate (50 mL) and water (50 mL). The aqueous layer was extracted with ethyl acetate (30 mL) for three times. The aqueous layer was basified with sodium carbonate (about 30 mL) till pH to 8, extracted with ethyl acetate (60 mL) twice. The combined organic layers were washed with water (80 mL) twice, dried over Na₂SO₄₍ₛ₎ and filtered. The filtrate was concentrated under reduced pressure to give the title compound (458 mg, 97% purity, 98% yield) as yellow solid. LC-MS (ESI): mass calcd. for C₂₄H₂₆F₃N₅O₂S 505.2, m/z found 506.5 [M+H]⁺. ¹H NMR (400 MHz, DMSO-d₆) δ 9.45 (br s, 1H), 8.04-7.98 (m, 1H), 7.95-7.87 (m, 1H), 7.21-7.15 (m, 1H), 7.06-6.98 (m, 2H), 5.88 (s, 1H), 4.22 (d, J=16.0 Hz, 1H), 4.10 (d, J=16.0 Hz, 1H), 3.98 (q, J=7.2 Hz, 2H), 3.80-3.68 (m, 1H), 3.28-3.22 (m, 2H), 3.16-3.11 (m, 1H), 3.05-2.95 (m, 2H), 2.74-2.66 (m, 1H), 2.58-2.54 (m, 1H), 2.44 (s, 3H), 1.05 (t, J=7.2 Hz, 3H).

### Compound 180: 2-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)propanoic acid (Single Diastereomer)

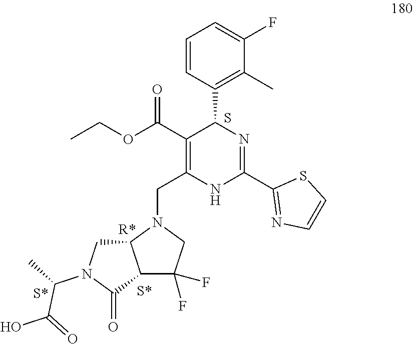

This compound was made using the procedure similar to Compound 42 by replacing T10-1 with T17-4 and replacing H5-1A with H2-1A. Purified by C18 column (acetonitrile:water (0.1% ammonium bicarbonate)=5% to 60%) to afford the desired product (29 mg, 98% purity) as yellow solids. LC-MS (ESI): mass calcd. for C₂₇H₂₈F₃N₅O₅S 591.2, m/z found 592.2 [M+H]⁺. ¹H NMR (400 MHz, CD₃OD) δ 7.89 (d, J=3.2 Hz, 1H), 7.70 (d, J=2.8 Hz, 1H), 7.19-7.13 (m, 2H), 6.99-6.92 (m, 1H), 5.99 (s, 1H), 4.66 (q, J=7.2 Hz, 1H), 4.39 (d, J=16.4 Hz, 1H), 4.17 (d, J=16.8 Hz, 1H), 4.08 (q, J=7.2 Hz, 2H), 3.98-3.94 (m, 1H), 3.83-3.80 (m, 1H), 3.70-3.66 (m, 1H), 3.58-3.50 (m, 1H), 3.45-3.35 (m, 1H), 3.13-2.98 (m, 1H), 2.52 (s, 3H), 1.47 (d, J=7.6 Hz, 3H), 1.14 (t, J=7.2 Hz, 3H).

### Compound 181A and 181B: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)-2-methylpropanoic acid (Single Diastereomer)

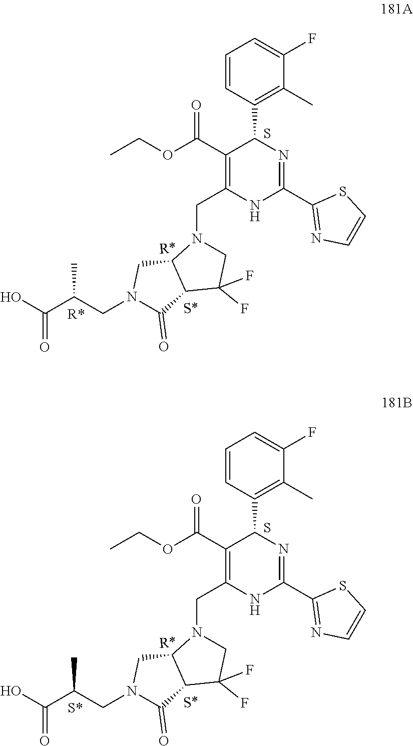

These two compounds were made using the procedure similar to Compound 42 by replacing tert-butyl 2,2-dimethyl-3-oxopropanoate with tert-butyl 2-methyl-3-oxopropanoate and replacing H5-1A with H2-1A.

181A: LC-MS (ESI): mass calcd. for C₂₈H₃₀F₃N₅O₅S 605.1 m/z found 606.2 [M+H]⁺. ¹H NMR (400 MHz, CDCl₃) δ 9.08 (s, 1H), 7.90 (d, J=3.2 Hz, 1H), 7.45 (d, J=3.6 Hz, 1H), 7.12-7.06 (m, 1H), 7.04-7.02 (m, 1H), 6.89 (t, J=0.8 Hz, 1H), 6.02 (s, 1H), 4.64 (d, J=14.8 Hz, 1H), 4.21 (t, J=12.4 Hz, 1H), 4.09-4.01 (m, 2H), 3.86 (d, J=10.4 Hz, 1H), 3.75 (d, J=14.8 Hz, 1H), 3.64-3.61 (m, 1H), 3.41-3.33 (m, 2H), 3.20 (t, J=11.6 Hz, 1H), 2.98-2.86 (m, 2H), 2.77-2.67 (m, 1H), 2.53 (s, 1.3H), 2.52 (s, 1.7H), 1.27 (d, J=6.4 Hz, 3H), 1.11 (t, J=7.2 Hz, 3H).

181B: LC-MS (ESI): mass calcd. for C₂₈H₃₀F₃N₅O₅S 605.1 m/z found 606.2 [M+H]⁺. ¹H NMR (400 MHz, CDCl₃) δ 8.85 (s, 1H), 7.80 (d, J=2.8 Hz, 1H), 7.44 (d, J=3.2 Hz, 1H), 7.13-7.08 (m, 1H), 7.03-7.01 (m, 1H), 6.92 (t, J=8.8 Hz, 1H), 6.01 (s, 1H), 4.62 (d, J=15.6 Hz, 1H), 4.07-4.00 (m, 2H), 3.78-3.69 (m, 3H), 3.59-3.45 (m, 3H), 3.39-3.32 (m, 1H), 3.31-3.25 (m, 1H), 2.93 (t, J=12.0 Hz, 1H), 2.75-2.65 (m, 1H), 2.52 (s, 1.3H), 2.51 (s, 1.7H), 1.21 (d, J=6.8 Hz, 3H), 1.09 (t, J=7.2 Hz, 3H).

### Compound 182A/B and 183A/B: 2-(((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)methyl)butanoic acid and 2-(((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-6-oxohexahydropyrrolo[3,4-b]pyrrol-5(1H)-yl)methyl)butanoic acid (Single Diastereomer)

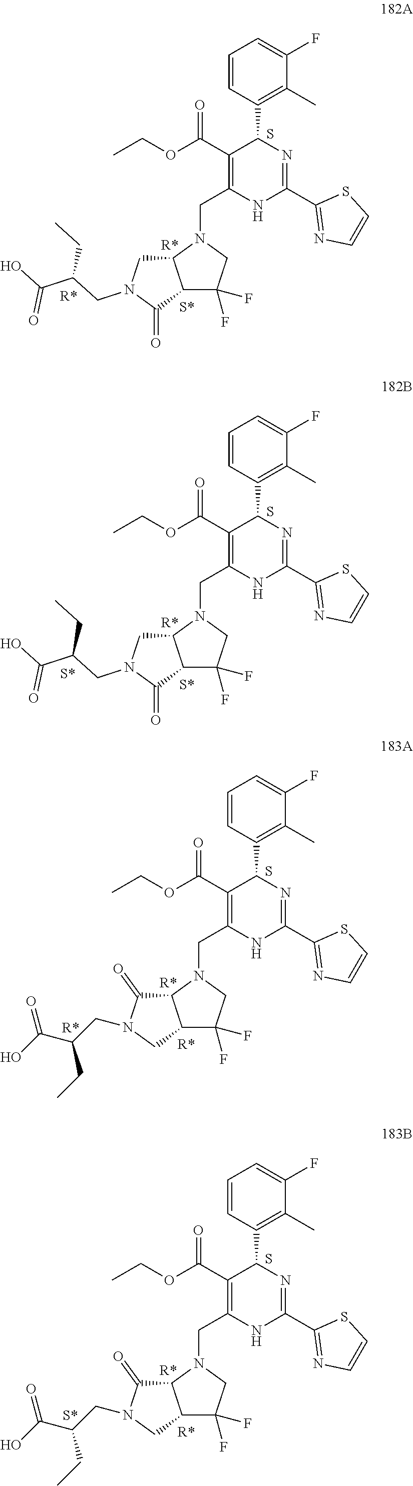

These compounds were made using the procedure similar to Compound 42 by replacing tert-butyl 2,2-dimethyl-3-oxopropanoate with tert-butyl 2-formylbutanoate and replacing H15-1A with H12-1A.

182A, LC-MS (ESI): mass calcd. for C₂₉H₃₂F₃N₅O₅S 619.2, m/z found 620.3 [M+H]⁺. ¹H NMR (400 MHz, CDCl₃) δ 8.88 (s, 1H), 7.78 (d, J=3.2 Hz, 1H), 7.45 (d, J=3.6 Hz, 1H), 7.11-7.09 (m, 1H), 7.03-7.01 (m, 1H), 6.94-6.90 (m, 1H), 6.01 (s, 1H), 4.62 (d, J=15.6 Hz, 1H), 4.06-4.00 (m, 2H), 3.79-3.73 (m, 3H), 3.58-3.49 (m, 2H), 3.39-3.25 (in, 3H), 2.93 (t, J=11.6 Hz, 1H), 2.71-2.65 (m, 1H), 2.52 (d, J=2.0 Hz, 3H), 1.75-1.51 (m, 2H), 1.09 (t, J=6.8 Hz, 3H), 1.00 (t, J=7.2 Hz, 3H).

182B, LC-MS (ESI): mass calcd. for C₂₉H₃₂F₃N₅O₅S 619.2, m/z found 620.2 [M+H]⁺. ¹H NMR (400 MHz, CDCl₃) δ 9.22 (s, 1H), 7.88 (d, J=3.2 Hz, 1H), 7.45 (d, J=3.6 Hz, 1H), 7.09-7.02 (m, 2H), 6.93-6.89 (in, 1H), 6.02 (s, 1H), 4.66 (d, J=14.8 Hz, 1H), 4.23 (t, J=12.8 Hz, 1H), 4.09-4.01 (m, 2H), 3.86 (d, J=10.4 Hz, 1H), 3.73 (d, J=14.4 Hz, 1H), 3.62-3.58 (m, 1H), 3.37-3.32 (m, 2H), 3.19 (t, J=11.6 Hz, 1H), 2.94-2.89 (m, 1H), 2.79-2.70 (m, 2H), 2.53 (d, J=2.0 Hz, 3H), 1.88-1.78 (m, 1H), 1.64-1.47 (m, 1H), 1.12 (t, J=7.2 Hz, 3H), 1.00 (t, J=7.6 Hz, 3H).

183A, LC-MS (ESI): mass calcd. for C₂₉H₃₂F₃N₅O₅S 619.2, m/z found 620.3 [M+H]⁺. ¹H NMR (400 MHz, CDCl₃) δ 8.87 (s, 1H), 7.93 (d, J=3.2 Hz, 1H), 7.45 (d, J=3.2 Hz, 1H), 7.12-7.08 (m, 1H), 7.04-7.02 (m, 1H), 6.93-6.89 (m, 1H), 6.01 (s, 1H), 4.69 (d, J=15.2 Hz, 1H), 4.30 (d, J=15.6 Hz, 1H), 4.32-4.02 (m, 2H), 3.91-3.87 (m, 1H), 3.75-3.70 (m, 1H), 360 (d, J=8.4 Hz, 1H), 3.50 (t, J=10.4 Hz, 1H), 3.26-3.17 (m, 3H), 3.05-2.99 (m, 1H), 2.94-2.87 (m, 1H), 2.53 (t, J=1.6 Hz, 3H), 1.73-1.62 (m, 1H), 1.52-1.45 (m, 1H), 1.13 (t, J=7.2 Hz, 3H), 0.96 (t, J=7.2 Hz, 3H).

183B, LC-MS (ESI): mass calcd. for C₂₉H₃₂F₃N₅O₅S 619.2, m/z found 620.3 [M+H]⁺. ¹H NMR (400 MHz, CDCl₃) δ 8.78 (s, 1H), 7.92 (d, J=3.2 Hz, 1H), 7.15 (d, J=3.2 Hz, 1H), 7.11-7.06 (m, 2H), 6.92-6.88 (m, 1H), 5.98 (s, 1H), 4.77 (d, J=16.0 Hz, 1H), 4.31 (d, J=16.0 Hz, 1H), 4.18 (t, J=12.8 Hz, 1H), 4.07-3.99 (m, 2H), 3.94-3.90 (m, 1H), 3.70-3.67 (m, 1H), 3.46-3.41 (m, 1H), 3.38-3.31 (m, 1H), 3.29-3.19 (m, 1H), 2.99-2.85 (m, 2H), 2.77-2.72 (m, 1H), 2.51 (d, J=1.6 Hz, 3H), 1.85-1.76 (m, 1H), 1.61-1.54 (m, 1H), 1.10 (t, J=7.2 Hz, 3H), 1.03 (t, J=7.6 Hz, 3H).

### Compound 184: 1-(((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)methyl)cyclopropane-1-carboxylic acid (Single Diastereomer)

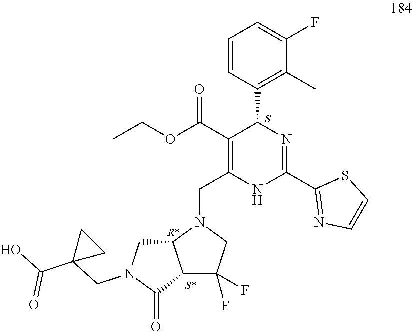

This compound was made using the procedure similar to Compound 42 by replacing T10-1 with T18 and replacing H5-1A with H2-1A. Purified by Prep. HPLC (Column: Gilson Xbrige C18 (5 μm 19*150 mm), Mobile Phase A: water (0.1% trifluoroacetic acid), Mobile Phase B: acetonitrile, UV: 214 nm, Flow rate: 15 mL/min, Gradient: 30-55% (% B)) and C18 column (acetonitrile:water (0.1% ammonium bicarbonate)=5% to 95%) to give the title compound (36 mg, 99.1% purity) as yellow solid. LC-MS (ESI): mass calcd. for C₂₉H₃₀F₃N₅O₅S 617.2, m/z found 618.2 [M+H]⁺. ¹H NMR (400 MHz, DMSO-d₆) δ 9.37 (br s, 1H), 8.01 (s, 0.6H), 7.94-7.90 (m, 1.4H), 7.22-7.17 (m, 1.3H), 7.08-7.01 (m, 1.7H), 5.90 (s, 0.7H), 5.78 (s, 0.3H), 4.26 (d, J=15.6 Hz, 0.7H), 4.13 (d, J=16.0 Hz, 0.7H), 4.04-3.91 (m, 2.6H), 3.82-3.77 (m, 1H), 3.57-3.41 (m, 6H), 3.03-2.93 (m, 1H), 2.44 (s, 2H), 2.40 (s, 1H), 1.09-0.99 (m, 5H), 0.93-0.84 (m, 2H).

### Compound 185A and 185B: 4-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-6-oxohexahydropyrrolo[3,4-b]pyrrol-5(1H)-yl)cyclohexane-1-carboxylic acid (Single Diastereomer)

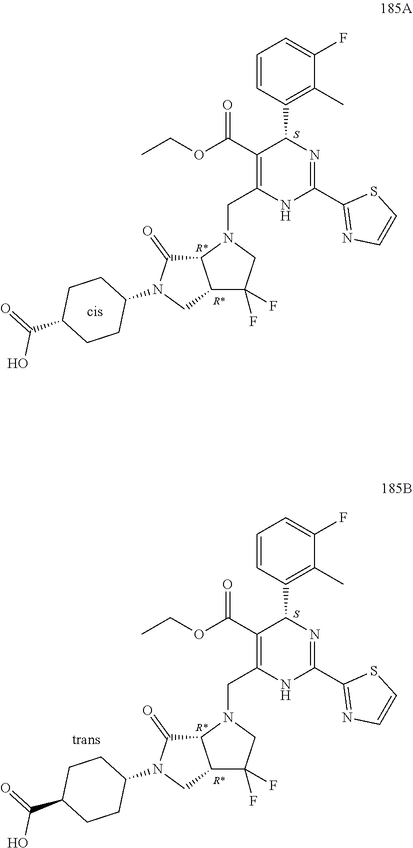

These two compound were made together with compound 44A/B.

185A, LC-MS (ESI): mass calcd. for C₃₁H₃₄F₃N₅O₅S 645.2, m/z found 646.2 [M+H]⁺. ¹H NMR (400 MHz, CD₃OD) δ 7.89 (d, J=3.2 Hz, 1H), 7.71 (d, J=3.2 Hz, 1H), 7.20-7.13 (m, 2H), 6.96-6.92 (m, 1H), 5.97 (s, 1H), 4.59 (d, J=16.8 Hz, 1H), 4.38 (d, J=16.8 Hz, 1H), 4.07 (q, J=7.2 Hz, 2H), 4.00-3.90 (m, 2H), 3.69 (dd, J=10.8, 2.8 Hz, 1H), 3.60-3.55 (m, 1H), 3.41-3.36 (m, 1H), 3.28-3.22 (m, 2H), 2.66 (br s, 1H), 2.52 (d, J=2.0 Hz, 3H), 2.28-2.21 (m, 2H), 1.80-1.64 (m, 6H), 1.15 (t, J=7.2 Hz, 3H).

185B, LC-MS (ESI): mass calcd. for C₃₁H₃₄F₃N₅O₅S 645.2, m/z found 646.2 [M+H]⁺. ¹H NMR (400 MHz, CD₃OD) δ 7.88 (d, J=3.2 Hz, 1H), 7.72 (d, J=3.2 Hz, 1H), 7.21-7.13 (m, 2H), 6.96-6.92 (m, 1H), 5.98 (s, 1H), 4.60 (d, J=16.8 Hz, 1H), 4.39 (d, J=16.8 Hz, 1H), 4.07 (q, J=7.2 Hz, 2H), 4.02-3.99 (m, 1H), 3.92-3.85 (m, 1H), 3.74 (dd, J=10.8, 2.8 Hz, 1H), 3.62-3.57 (m, 1H), 3.41-3.37 (m, 1H), 3.28-3.20 (m, 2H), 2.52 (d, J=2.0 Hz, 3H), 2.31-2.25 (m, 1H), 2.13-2.07 (m, 2H), 1.85-1.84 (m, 2H), 1.66-1.53 (m, 4H), 1.15 (t, J=7.2 Hz, 3H).

### Compound 186: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3-fluoro-4-oxohexahydropyrrolo[3,4-b]pyrrol-5(1H)-yl)-2,2-dimethylpropanoic acid

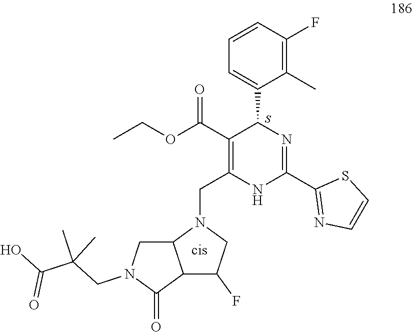

This compound was made using the procedure similar to Compound 42 by replacing T4 with T19 and replacing H5-1A with H2-1A. Purified by Prep-HPLC (Column: Gilson Xbrige C18 (5 μm 19*150 mm), Mobile phase A: water (0.1% ammonium bicarbonate), Mobile phase B: acetonitrile, UV: 214 nm, Flow rate: 15 mL/min, Gradient: 10-15% (% B)) to give the title compound (30 mg, 99.3% purity) as yellow solids. LC-MS (ESI): mass calcd. For C₂₉H₃₃F₂N₅O₅S 601.2, m/z found 602.2 [M+H]⁺. ¹H NMR (400 MHz, CDCl₃) δ 9.24 (s, 0.5H), 9.03 (s, 0.5H), 7.92 (d, J=3.2 Hz, 0.5H), 7.83 (d, J=3.6 Hz, 0.5H), 7.44 (d, J=2.4 Hz, 1H), 7.13-6.89 (m, 3H), 6.02 (s, 0.5H), 5.98 (s, 0.5H), 5.50-5.27 (m, 1H), 4.69-4.64 (m, 0.5H), 4.56-4.52 (m, 0.5H), 4.11-3.95 (m, 3H), 3.78-3.24 (m, 5H), 3.11-2.92 (m, 2H), 2.83-2.73 (m, 1H), 2.54 (s, 3H), 1.32-1.31 (m, 3H), 1.26-1.25 (m, 3H), 1.14-1.10 (m, 3H).

### Preparation of Intermediate T24

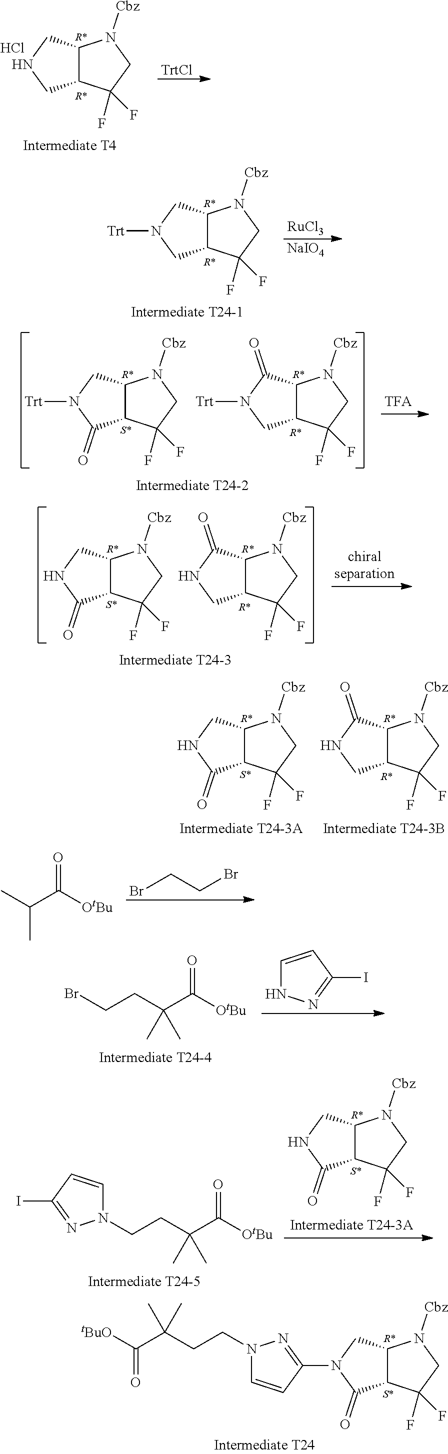

### T24-1: (cis)-Benzyl 3,3-difluoro-5-tritylhexahydropyrrolo[3,4-b]pyrrole-1(2H)-carboxylate

To a solution of T4 (4.1 g, 90% purity, 11.6 mmol) in dichloromethane (20 mL) was added (chloromethanetriyl)tribenzene (4.3 g, 15.4 mmol) and diisopropylethylamine (4.5 g, 34.8 mmol) at room temperature. After stirring at room temperature overnight, the reaction mixture was diluted with water (20 mL) and extracted with dichloromethane (100 mL) twice. The combined organic layers were washed with brine (100 mL), dried over Na₂SO₄₍ₛ₎, filtered and concentrated. The residue was purified by silica gel column chromatography (petroleum ether:ethyl acetate=20:1 to 10:1) to afford the title compound (6.7 g, 90% purity from ¹H NMR, 99% yield) as light yellow oil. ¹H NMR (400 MHz, CDCl₃) δ 7.45-7.38 (m, 9H), 7.28-7.11 (m, 11H), 5.32-5.29 (m, 0.5H), 5.16-5.12 (m, 1.5H), 4.42-4.07 (m, 3H), 3.37-3.29 (m, 1.5H), 3.21-3.18 (m, 0.5H), 2.82-2.71 (m, 1H), 1.78-1.73 (m, 1H), 1.65-1.62 (m, 1H).

### T24-2: Mixture of (cis)-benzyl 3,3-difluoro-4-oxo-5-tritylhexahydropyrrolo[3,4-b]pyrrole-1(2H)-carboxylate and (cis)-benzyl 3,3-difluoro-4-oxo-5-tritylhexahydropyrrolo[3,4-b]pyrrole-1(2H)-carboxylate

To a solution of T24-1 (6.7 g, 90% purity, 11.5 mmol) in ethyl acetate (20 mL) and water (20 mL) was added sodium periodate (5 g, 23.4 mmol) and ruthenium(III) chloride (100 mg, 0.482 mmol) at room temperature. After stirred at room temperature overnight, the reaction mixture was diluted with water (20 mL) and extracted with ethyl acetate (100 mL) twice. The combined organic layers were washed with brine (100 mL), dried over Na₂SO₄₍ₛ₎, filtered and concentrated. The residue was purified by silical gel column chromatography (petroleum ether:ethyl acetate=4:1 to 3:1) to give a mixture compound (5.8 g, 90% purity from ¹H NMR, 84% yield) as yellow oil. ¹H NMR (400 MHz, CDCl₃) δ 7.38-7.14 (m, 20H), 5.26-5.01 (m, 2H), 4.96-4.81 (m, 0.5H), 4.58-4.44 (m, 0.5H), 4.04-3.77 (m, 1H), 3.70-3.31 (m, 3.5H), 3.20-3.03 (m, 0.5H).

### T24-3: Mixture of (cis)-benzyl 3,3-difluoro-4-oxohexahydropyrrolo[3,4-b]pyrrole-1(2H)-carboxylate and (cis)-benzyl 3,3-difluoro-4-oxohexahydropyrrolo[3,4-b]pyrrole-1(2H)-carboxylate

To a solution of T24-2 (5.8 g, 90% purity, 9.692 mmol) in dichloromethane (20 mL) was added trifluoroacetic acid (10 mL) at room temperature. After stirred at room temperature under nitrogen atmosphere for 2 hours, the reaction mixture was diluted with water (20 mL) and extracted with ethyl acetate (100 mL) twice. The combined extracts were washed with brine (50 mL), dried over Na₂SO₄₍ₛ₎, filtered and concentrated. The residue was purified by C18 column (acetonitrile:water=60% to 80%) to give a mixture compound (2.8 g, 90% purity from ¹H NMR, 88% yield) as white solids. LC-MS (ESI): mass calcd. for C₁₄H₁₄F₂N₂O₃ 296.1, m/z found 297.3 [M+H]⁺. ¹H NMR (400 MHz, CDCl₃) δ 7.43-7.36 (m, 4.5H), 6.87-6.74 (m, 0.5H), 5.29-5.11 (m, 2H), 5.00-4.96 (m, 0.3H), 4.88-4.82 (m, 0.2H), 4.78-4.68 (m, 0.5H), 4.25-3.95 (m, 1H), 3.79-3.44 (m, 3H), 3.38-3.19 (m, 1H).

### T24-3A and T24-3B: (cis)-Benzyl 3,3-difluoro-4-oxohexahydropyrrolo[3,4-b]pyrrole-1(2H)-carboxylate and (cis)-benzyl 3,3-difluoro-4-oxohexahydropyrrolo[3,4-b]pyrrole-1(2H)-carboxylate

A mixture of T24-3 (2.8 g, 90% purity, 8.51 mmol) was separated by chiral Prep. HPLC (Chiralpak IG 5 μm 20*250 mm; Mobile Phase: Hex:EtOH=40:60 at 15 mL/min; Temp: 35° C.; Wavelength: 214 nm) to give the title compound T24-3A (1 g, 90% purity from ¹H NMR, 36% yield) and T24-3B (1.1 g, 90% purity from ¹H NMR, 39% yield) as white solids.

T24-3A: LC-MS (ESI): mass calcd. for C₁₄H₁₄F₂N₂O₃ 296.1, m/z found 297.4 [M+H]⁺. Chiral analysis (Column: Chiralpak IG 5 μm 4.6*250 mm; Mobile Phase: Hex:EtOH=40:60 at 1 mL/min; Temp: 30° C.; Wavelength: 214 nm, R_{T}=7.722 min). ¹H NMR (400 MHz, CDCl₃) δ 7.42-7.32 (m, 5H), 6.27-6.22 (m, 1H), 5.24-5.12 (m, 2H), 4.81-4.69 (m, 1H), 4.15-3.96 (m, 1H), 3.83-3.45 (m, 3H), 3.36-3.29 (m, 1H).

T24-3B: LC-MS (ESI): mass calcd. for C₁₄H₁₄F₂N₂O₃ 296.1, m/z found 297.4 [M+H]⁺. Chiral analysis (Column: Chiralpak IG 5 μm 4.6*250 mm; Mobile Phase: Hex:EtOH=40:60 at 1 mL/min; Temp: 30° C.; Wavelength: 214 nm, R_{T}=9.848 min). ¹H NMR (400 MHz, CDCl₃) δ 7.51-7.36 (m, 5H), 6.57 (s, 0.5H), 6.20 (s, 0.5H), 5.28-5.15 (m, 2H), 4.99 (s, 0.5H), 4.84 (s, 0.5H), 4.28-4.09 (m, 1H), 3.65-3.55 (m, 3H), 3.34-3.28 (m, 1H).

### T24-4: tert-Butyl-4-bromo-2,2-dimethylbutanoate

To a solution of tert-butyl isobutyrate (10 g, 69.3 mmol) in tetrahydrofuran (10 mL) was added 2 M lithium diisopropylamide in tetrahydrofuran (40 mL, 80 mmol) at −70° C. under nitrogen atmosphere. After stirred at −70° C. for 1 hour, 1,2-dibromoethane (14 g, 74.5 mmol) was added at −70° C. and warmed slowly to room temperature overnight. And then the reaction mixture was quenched with saturation ammonium chloride (50 mL) and extracted with ethyl acetate (60 mL) twice. The combined organic layers were washed with brine (30 mL), dried over Na₂SO₄₍ₛ₎, filtered and concentrated. The residue was purified by silica gel column chromatography (petroleum ether:ethyl acetate=50:1) to give the desired compound (8 g, 80% purity from ¹H NMR, 37% yield) as colorless oil. ¹H NMR (400 MHz, CDCl₃) δ 3.36-3.32 (m, 2H), 2.13-2.08 (m, 2H), 1.45 (s, 9H), 1.16 (s, 6H).

### T24-5: tert-Butyl4-(3-iodo-1H-pyrazol-1-yl)-2,2-dimethylbutanoate

To the solution of 3-iodo-1H-pyrazole (1 g, 5.16 mmol) in acetonitrile (15 mL) was added cesium carbonate (3.4 g, 10.4 mmol) and tert-butyl 4-bromo-2,2-dimethylbutanoate T24-4 (3.2 g, 80% purity, 10.2) at room temperature. After stirred at 80° C. under nitrogen atmosphere overnight, the reaction mixture was cooled to room temperature, diluted with water (10 mL) and extracted with ethyl acetate (100 mL) twice. The combined organic layers were washed with brine (50 mL), dried over Na₂SO₄₍ₛ₎, filtered and concentrated to give a residue, which was purified by silica gel column chromatography (petroleum ether:ethyl acetate=4:1 to 2:1) to afford the title compound (700 mg, 90% purity from ¹H NMR, 34% yield) as yellow oil. LC-MS (ESI): mass calcd. for C₁₃H₂₁₁N₂O₂ 364.1, m/z found 365.4 [M+H]⁺. ¹H NMR (400 MHz, CDCl₃) δ 7.49 (s, 0.3H), 7.20 (s, 0.7H), 6.39 (s, 1H), 4.23-4.11 (m, 2H), 2.08-1.98 (m, 2H), 1.48 (s, 3H), 1.45 (s, 6H), 1.22 (s, 2H), 1.18 (s, 4H).

### T24: (cis)-Benzyl 5-(1-(4-(tert-butoxy)-3,3-dimethyl-4-oxobutyl)-1H-pyrazol-3-yl)-3,3-difluoro-4-oxohexahydropyrrolo[3,4-b]pyrrole-1(2H)-carboxylate

To a solution of tert-butyl 4-(3-iodo-1H-pyrazol-1-yl)-2,2-dimethylbutanoate T24-5 (200 mg, 90% purity, 0.494 mmol) in 1,4-dioxane (6 mL) was added potassium phosphate (205 mg, 0.966 mmol) and T24-3A (105 mg, 90% purity, 0.319 mmol) under nitrogen atmosphere. The reaction mixture was stirred at 20° C. under nitrogen atmosphere for 10 minutes, then copper(l) iodide (92 mg, 0.483 mmol) and (1R,2R)—N,N-dimethyl-1,2-cyclohexanediamine (69 mg, 0.485 mmol) were added. After stirred at 80° C. under nitrogen atmosphere overnight, the reaction mixture was cooled to room temperature, diluted with water (10 mL) and extracted with ethyl acetate (60 mL) twice. The combined organic layers were washed with brine (30 mL), dried over Na₂SO₄₍ₛ₎, filtered and concentrated to give a residue, which was purified by C18 column (acetonitrile:water=40% to 70%) to give the title compound (105 mg, 90% purity from ¹H NMR, 36% yield) as light yellow oil. LC-MS (ESI): mass calcd. for C₂₇H₃₄F₂N₄O₅ 532.2, m/z found 533.5 [M+H]⁺. ¹H NMR (400 MHz, CDCl₃) δ 7.39-7.30 (m, 6H), 6.81 (s, 1H), 5.21-5.13 (m, 2H), 4.84-4.72 (m, 1H), 4.23-4.07 (m, 2H), 4.04-3.98 (m, 3H), 3.79-3.67 (m, 1H), 3.59-3.52 (m, 1H), 2.05-2.01 (m, 2H), 1.46 (s, 9H), 1.19 (s, 6H).

### Compound 187: 4-(3-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)-1H-pyrazol-1-yl)-2,2-dimethylbutanoic acid (Single Diastereomer)

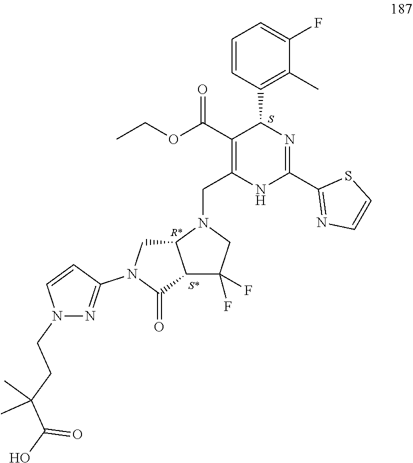

This compound was made using the procedure similar to Compound 42 by replacing T10-3A with T24 and replacing H5-1A with H2-1A. Purified by C18 column (acetonitrile:water=60% to 80%) to give the desired compound (62.2 mg, 96.7% purity) as yellow solids. LC-MS (ESI): mass calcd. for C₃₃H₃₆F₃N₇O₅S 699.2, m/z found 700.1 [M+H]⁺. ¹H NMR (400 MHz, CD₃OD) δ 7.77 (s, 1H), 7.64 (s, 1H), 7.45 (s, 1H), 7.21-7.17 (m, 2H), 6.98-6.93 (m, 1H), 6.74 (d, J=2.4 Hz, 1H), 5.98 (s, 1H), 4.46-4.35 (m, 1H), 4.28-4.19 (m, 2H), 4.17-3.99 (m, 6H), 3.80-3.68 (m, 1H), 3.51-3.42 (m, 1H), 3.18-3.06 (m, 1H), 2.51 (s, 3H), 1.95-1.80 (m, 2H), 1.15-1.10 (m, 9H).

### Preparation of Intermediate T13

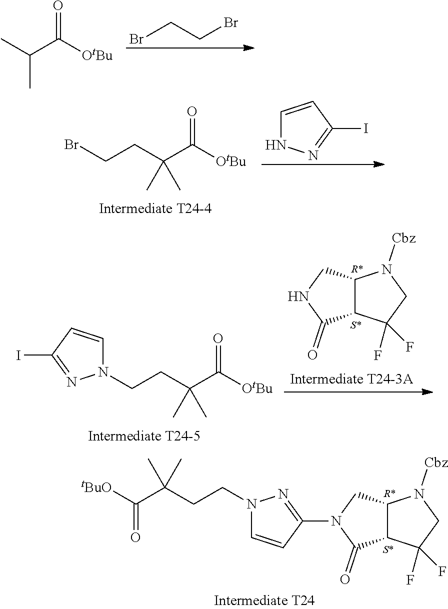

### T13-1: tert-Butyl 3-isocyanato-2,2-dimethylpropanoate

To a solution of tert-butyl 3-amino-2,2-dimethylpropanoate (600 mg, 90% purity, 3.12 mmol) in dichloromethane (5 mL) and saturated sodium carbonate aqueous solution (6 mL) was added a solution of triphosgene (510 mg, 1.72 mmol) in dichloromethane (1 mL) at 0° C.-5° C. The mixture was stirred at room temperature overnight. Then it was washed with 20% wt aqueous sodium carbonate (10 mL) and brine (10 mL). The organic layer was dried over Na₂SO₄₍ₛ₎ and filtered. The filtrate was concentrated to give the title compound (600 m g, 90% purity from ¹H NMR, 87% yield) as yellow oil. ¹H NMR (400 MHz, CDCl₃) δ 3.35 (s, 2H), 1.46 (s, 9H), 1.19 (s, 6H).

### T13: (cis)-Benzyl 5-((3-(tert-butoxy)-2,2-dimethyl-3-oxopropyl)carbamoyl)-3,3-difluorohexahydropyrrolo[3,4-b]pyrrole-1(2H)-carboxylate

To a solution of T4 (770 mg, 90% purity, 2.17 mmol) and triethylamine (500 mg, 4.94 mmol) in dichloromethane (10 mL) was added a solution of tert-butyl 3-isocyanato-2,2-dimethylpropanoate T13-1 (650 mg, 90% purity, 2.94 mmol) in dichloromethane (2 mL) at 0° C. After stirred at room temperature for 1 hour, the reaction mixture was concentrated at room temperature to give a residue, which was purified by C18 column (acetonitrile:water=35% to 95%) to give the title compound (970 mg, 90% purity from ¹H NMR, 83% yield) as yellow oil. ¹H NMR (400 MHz, CDCl₃) δ 7.40-7.32 (m, 5H), 5.20-5.11 (m, 2H), 5.01-4.99 (m, 1H), 4.61-4.56 (m, 1H), 4.07-3.91 (m, 1H), 3.79-3.53 (m, 5H), 3.30-3.28 (m, 2H), 3.16 (br s, 1H), 1.44 (s, 9H), 1.14 (s, 6H).

### Compound 188: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxooctahydropyrrolo[3,4-b]pyrrole-5-carboxamido)-2,2-dimethylpropanoic acid (Single Diastereomer)

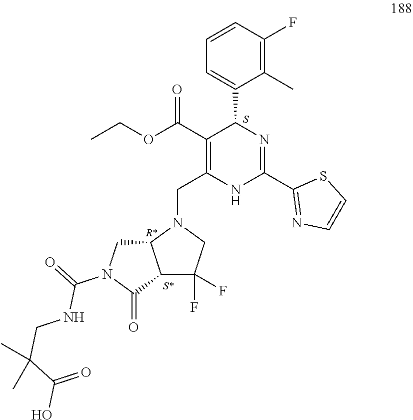

This compound was made using the procedure similar to Compound 42 by replacing T10-1 with T13 and replacing H5-1A with H2-1A. Purified by C18 column (acetonitrile:water=35% to 70%) to give the title compound (76.6 mg, 99.1% purity) as yellow solids. LC-MS (ESI): mass calcd. for C₃₀H₃₃F₃N₆O₆S 662.68, m/z found 663.3 [M+H]⁺. ¹H NMR (400 MHz, CDCl₃) δ 9.10 (br s, 1H), 8.71 (t, J=6.0 Hz, 1H), 7.78 (d, J=3.6 Hz, 1H), 7.42-7.41 (m, 1H), 7.12 (q, J=8.0 Hz, 1H), 7.04 (d, J=8.0 Hz, 1H), 6.92 (t, J=8.8 Hz, 1H), 6.01 (s, 1H), 4.45-4.40 (m, 1H), 4.10-4.00 (m, 4H), 3.92-3.87 (m, 1H), 3.80-3.77 (m, 1H), 3.67-3.60 (m, 1H), 3.55-3.50 (m, 1H), 3.47-3.39 (m, 2H), 2.97-2.88 (m, 1H), 2.51 (s, 3H), 1.25 (s, 3H), 1.23 (s, 3H), 1.11 (t, J=7.2 Hz, 3H).

### Compound 189: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-6-oxooctahydropyrrolo[3,4-b]pyrrole-5-carboxamido)-2,2-dimethylpropanoic acid (Single Diastereomer)

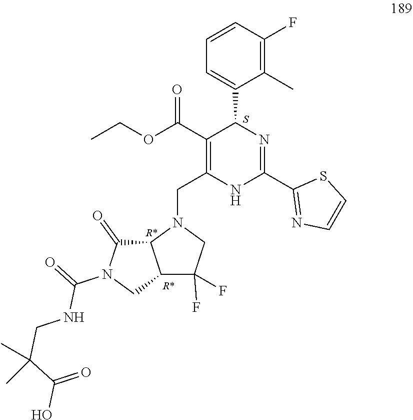

This compound was made together with compound 188. LC-MS (ESI): mass calcd. for C₃₀H₃₃F₃N₆O₆S 662.68, m/z found 663.3 [M+H]⁺. ¹H NMR (400 MHz, CDCl₃) δ 9.07 (br s, 1H), 8.63 (t, J=6.0 Hz, 1H), 7.84 (d, J=3.2 Hz, 1H), 7.42-7.41 (m, 1H), 7.12-7.05 (m, 2H), 6.93-6.88 (m, 1H), 6.01 (s, 1H), 4.61 (d, J=16.4 Hz, 1H), 4.40 (d, J=17.2 Hz, 1H), 4.22 (dd, J=12.4, 3.6 Hz, 1H), 4.11-4.00 (m, 3H), 3.96-3.90 (m, 1H), 3.48-3.38 (m, 2H), 3.35-3.18 (m, 3H), 2.53 (s, 3H), 1.21 (s, 3H), 1.18 (s, 3H), 1.12 (t, J=7.2 Hz, 3H).

### Preparation of Intermediate T14

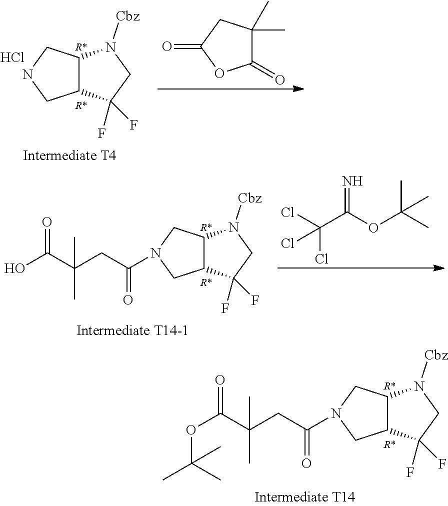

### T14-1: 4-((cis)-1-((Benzyloxy)carbonyl)-3,3-difluorohexahydropyrrolo[3,4-b]pyrrol-5(1H)-yl)-2,2-dimethyl-4-oxobutanoic acid

To a solution of T4 (750 mg, 2.35 mmol) and triethylamine (2.5 mL, 18.0 mmol) in tetrahydrofuran (30 mL) was added 2,2-dimethylsuccinic anhydride (750 mg, 5.85 mmol) at 0° C. After stirred at room temperature overnight, the reaction was quenched with 2 M hydrochloride aqueous solution (50 mL) and extracted with dichloromethane (50 mL) twice. The combined extracts were washed with brine (150 mL), dried over Na₂SO₄₍ₛ₎, filtered and concentrated to give the title compound (1.0 g, 90% purity from ¹H NMR, 93% yield) as yellow solids. LC-MS (ESI): mass calcd. for C₂₀H₂₄F₂N₂O₅ 410.12, m/z found 411.5 [M+H]⁺. ¹H NMR (400 MHz, CDCl₃) δ 7.42-7.29 (m, 5H), 5.19-5.09 (m, 2H), 4.63-4.51 (m, 1H), 4.04-3.60 (m, 6H), 3.35-3.10 (m, 1H), 2.63-2.32 (m, 2H), 1.32-1.24 (m, 6H).

### T14: (cis)-Benzyl 5-(4-(tert-butoxy)-3,3-dimethyl-4-oxobutanoyl)-3,3-difluorohexahydropyrrolo[3,4-b]pyrrole-1(2H)-carboxylate

To a solution of T14-1 (700 mg, 90% purity, 1.54 mmol) and tert-butyl 2,2,2-trichloroacetimidate (670 mg, 3.07 mmol) in dichloromethane (8 mL) and hexane (8 mL) was added boron trifluoride etherate (100 mg, 0.705 mmol) at 0° C. The reaction mixture was stirred at room temperature overnight. Sodium bicarbonate (1.0 g) and sodium sulfate anhydrous (1.0 g) was added to quench the reaction. The reaction mixture was stirred at room temperature for 0.5 hour and filtrated. The cake was washed with dichloromethane (20 mL), and the filtrate was concentrated to give the crude product, which was purified by C18 column (acetonitrile:water (0.1% ammonium bicarbonate)=5% to 70%) to give the title compound (490 mg, 90% purity from ¹H NMR, 62% yield) as colorless oil. LC-MS (ESI): mass calcd. for C₂₄H₃₂F₂N₂O₅ 466.2, m/z found 467.4 [M+H]⁺. ¹H NMR (400 MHz, CDCl₃) δ 7.43-7.29 (m, 5H), 5.28-5.06 (m, 2H), 4.62-4.49 (m, 1H), 4.10-3.58 (m, 6H), 3.31-3.06 (m, 1H), 2.47-2.43 (m, 2H), 1.43 (s, 9H), 1.25-1.22 (m, 6H).

### Compound 190: 4-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)-2,2-dimethyl-4-oxobutanoic acid (Single Diastereomer)

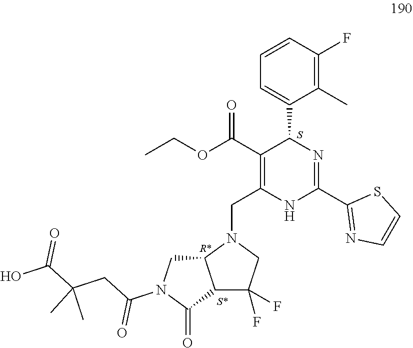

This compound was made using the procedure similar to Compound 42 by replacing T10-1 with T14 and replacing H5-1A with H2-1A. Purified by C18 column (acetonitrile:water (0.1% ammonium bicarbonate)=5% to 60%) to give the title product (30 mg, 97.9% purity) as yellow solids. LC-MS (ESI): mass calcd. for C₃₀H₃₂F₃N₅O₆S 647.2, m/z found 648.3 [M+H]⁺. ¹H NMR (400 MHz, CDCl₃) δ 8.88 (s, 1H), 7.94 (d, J=3.6 Hz, 1H), 7.40 (d, J=3.2 Hz, 1H), 7.16-7.11 (m, 1H), 7.01-6.99 (m, 1H), 6.92 (t, J=8.8 Hz, 1H), 6.01 (s, 1H), 4.48 (d, J=16.4 Hz, 1H), 4.09-3.98 (m, 4H), 3.79-3.65 (m, 3H), 3.62-3.55 (m, 1H), 3.35 (t, J=12.4 Hz, 1H), 2.87-2.77 (m, 2H), 2.52 (s, 1.5H), 2.51 (s, 1.5H), 1.34 (s, 3H), 1.26 (s, 3H), 1.11 (t, J=7.2 Hz, 3H).

### Compound 191: 4-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-6-oxohexahydropyrrolo[3,4-b]pyrrol-5(1H)-yl)-2,2-dimethyl-4-oxobutanoic acid (Single Diastereomer)

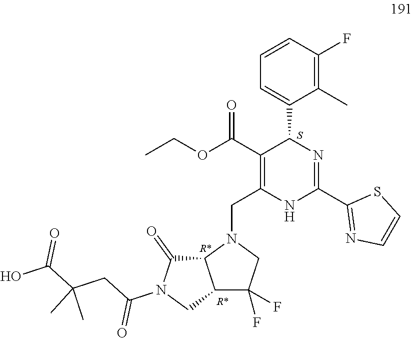

This compound was made together with compound 190. LC-MS (ESI): mass calcd. for C₃₀H₃₂F₃N₅O₆S 647.2, m/z found 648.3 [M+H]⁺. ¹H NMR (400 MHz, CDCl₃) δ 9.02 (s, 1H), 7.83 (d, J=3.2 Hz, 1H), 7.40 (d, J=3.2 Hz, 1H), 7.13-7.05 (m, 2H), 6.91 (t, J=8.8 Hz, 1H), 6.00 (s, 1H), 4.69 (d, J=16.0 Hz, 1H), 4.33 (d, J=16.0 Hz, 1H), 4.16-4.01 (m, 3H), 3.95-3.86 (m, 2H), 3.53 (d, J=16.8 Hz, 1H), 3.38 (q, J=10.4 Hz, 1H), 3.27-3.12 (m, 2H), 2.90 (d, J=17.2 Hz, 1H), 2.53 (s, 3H), 1.34 (s, 3H), 1.24 (s, 3H), 1.12 (t, J=7.2 Hz, 3H).

### Compound 192: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-6-oxohexahydropyrrolo[3,4-b]pyrrol-5(1H)-yl)cyclobutane-1-carboxylic acid (Single Diastereomer)

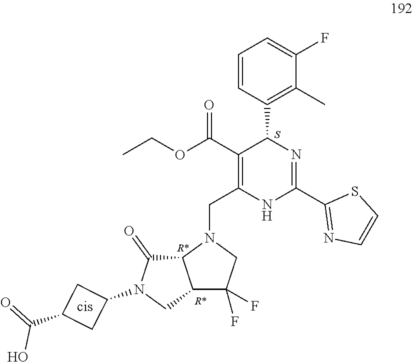

This compound was made together with compound 40. LC-MS (ESI): mass calcd. for C₂₉H₃₀F₃N₅O₅S 617.2, m/z found 618.3 [M+H]⁺. ¹H NMR (400 MHz, CD₃OD) δ 7.92-7.90 (m, 1H), 7.72-7.70 (m, 1H), 7.20-7.13 (m, 2H), 6.96-6.92 (m, 1H), 5.97 (s, 1H), 4.58-4.47 (m, 2H), 4.39 (d, J=16.8 Hz, 1H), 4.09-4.04 (m, 2H), 3.99 (d, J=8.4 Hz, 1H), 3.86 (d, J=10.8 Hz, 1H), 3.73-3.68 (m, 1H), 3.44-3.37 (m, 1H), 3.29-3.24 (m, 2H), 2.93-2.83 (m, 1H), 2.55-2.38 (m, 7H), 1.15 (t, J=7.2 Hz, 3H).

### Compound 193: 4-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)-2,2-dimethylbutanoic acid (Single Diastereomer)

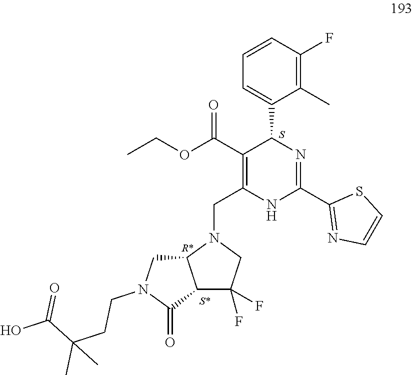

This compound was made using the procedure similar to Compound 42 by replacing tert-butyl 2,2-dimethyl-3-oxopropanoate with tert-butyl 2,2-dimethyl-4-oxobutanoate and replacing H5-1A with H2-1A. Purified by C18 column (acetonitrile:water (0.1% ammonium bicarbonate)=5% to 95%) to give the title compound (50 mg, 97.0% purity) as yellow solids. LC-MS (ESI): mass calcd. for C₃₀H₃₄F₃N₅O₅S 633.7, m/z found 634.3 [M+H]⁺. ¹H NMR (400 MHz, DMSO-d₆) δ 9.36 (s, 1H), 8.01 (s, 0.5H), 7.93-7.90 (m, 1.5H), 7.21-7.16 (m, 1.3H), 7.08-7.01 (m, 1.7H), 5.89 (s, 0.7H), 5.78 (s, 0.3H), 4.27-4.14 (m, 1.5H), 4.00-3.95 (m, 2.5H), 3.80-3.76 (m, 1H), 3.50-3.38 (m, 4H), 3.24-3.17 (m, 1.3H), 3.04-2.94 (m, 1.7H), 2.45-2.40 (m, 3H), 1.64-1.52 (m, 2H), 1.09-1.02 (m, 4.6H), 0.98-0.93 (m, 4.4H).

### Compound 194: 3-(((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)sulfonyl)cyclobutane-1-carboxylic acid (Single Diastereomer)

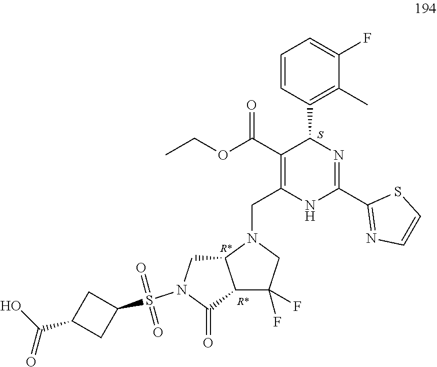

This compound was made using the procedure similar to Compound 46 by replacing tert-butyl 3-(chlorosulfonyl)-2,2-dimethylpropanoate with tert-butyl 3-(chlorosulfonyl)cyclobutane-1-carboxylate. Purified by C18 column (acetonitrile:water=30% to 60%) to give the title compound (15.3 mg, 97.7% purity) as yellow solids. LC-MS (ESI): mass calcd. for C₂₉H₃₂F₃N₅O₆S₂ 667.2, m/z found 668.3 [M+H]⁺. ¹H NMR (400 MHz, CD₃OD) δ 7.93 (d, J=3.2 Hz, 1H), 7.71 (d, J=3.2 Hz, 1H), 7.5-7.12 (m, 2H), 6.94-6.89 (m, 1H), 5.97 (s, 1H), 4.26 (d, J=17.2 Hz, 1H), 4.14 (d, J=16.4 Hz, 1H), 4.05 (q, J=7.2 Hz, 2H), 4.01-3.92 (m, 1H), 3.88-3.79 (m, 2H), 3.64 (d, J=11.2 Hz, 1H), 3.48-3.40 (m, 2H), 3.35-3.32 (m, 1H), 3.26-3.17 (m, 1H), 3.12-3.01 (m, 1H), 2.97-2.88 (m, 1H), 2.63 (q, J=10.0 Hz, 2H), 2.56-2.41 (m, 5H), 1.12 (t, J=7.2 Hz, 3H).

### Compound 195: 2-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-51H)-yl)-2-methylpropanoic acid (Single Diastereomer)

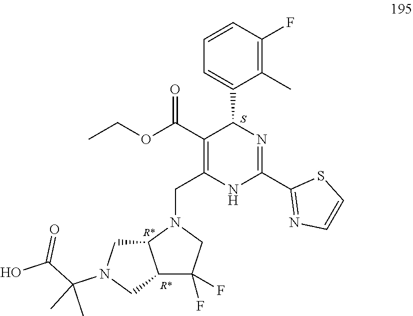

This compound was made using the procedure similar to Compound 14 by replacing tert-butyl 2-bromopropanoate with tert-butyl 2-bromo-2-methylpropanoate and replacing T17-3 with T4 using K₂CO₃/NaI/MeCN. Purified by C18 column (acetonitrile:water (+0.2% ammonium bicarbonate)=40% to 60%) to give the title compound (21.5 mg, 99.5% purity) as yellow solids. LC-MS (ESI): mass calcd. for C₂₈H₃₂F₃N₅O₄S 591.2, m/z found 591.9 [M+H]⁺. ¹H NMR (400 MHz, CDCl₃) δ 9.40 (s, 1H), 7.89 (d, J=3.2 Hz, 1H), 7.41 (d, J=2.8 Hz, 1H), 7.09-7.04 (m, 1H), 6.99-6.97 (m, 1H), 6.93-6.88 (m, 1H), 6.01 (s, 1H), 4.50 (d, J=16.8 Hz, 1H), 4.10-3.98 (m, 2H), 3.90 (d, J=16.8 Hz, 1H), 3.68-3.65 (m, 1H), 3.37-3.31 (m, 1H), 3.24-3.18 (m, 2H), 3.09-2.93 (m, 2H), 2.72-2.61 (m, 2H), 2.53 (d, J=1.2 Hz, 3H), 1.35 (s, 3H), 1.34 (s, 3H), 1.10 (t, J=7.2 Hz, 3H).

### Preparation of Intermediate T22

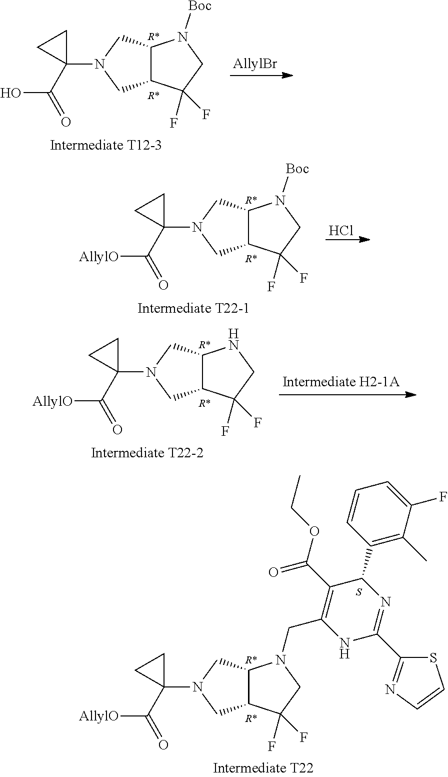

### T22-1: (cis)-tert-Butyl 5-(1-((allyloxy)carbonyl)cyclopropyl)-3,3-difluorohexahydropyrrolo[3,4-b]pyrrole-1(2H)-carboxylate

To a solution of T12-3 (350 mg, 90% purity, 0.949 mmol) in N,N-dimethylformamide (5 mL) was added potassium carbonate (528 mg, 2.85 mmol) and 3-bromoprop-1-ene (137 mg, 1.14 mmol). The mixture was stirred for 2 hours at room temperature. The mixture was concentrated under reduced pressure, the resulting residue was purified by column chromatography on silica gel (petroleum ether:ethyl acetate=3:1) to give the desired product (220 mg, 90% purity from ¹H NMR, 56% yield) as colorless oil. LC-MS (ESI): mass calcd. for C₁₈H₂₆F₂N₂O₄ 372.2, m/z found 373.4 [M+H]⁺. ¹H NMR (400 MHz, CDCl₃) δ 5.89-5.84 (m, 1H), 5.29-5.21 (m, 2H), 4.55 (d, J 5.2 Hz, 2H), 4.44-4.35 (m, 1H), 4.98-4.80 (m, 1H), 3.46-2.96 (m, 6H), 1.45 (s, 9H), 1.32-1.25 (m, 2H), 0.96-0.90 (m, 2H).

### T22-2: Allyl 1-((cis)-3,3-difluorohexahydropyrrolo[3,4-b]pyrrol-5(1H)-yl)cyclopropanecarboxylate hydrochloride

A solution of T22-1 (220 mg, 90% purity, 1.79 mmol) in 4 M hydrochloride in ethyl acetate (5 mL) was stirred at room temperature for 1 hour under nitrogen atmosphere. The mixture was concentrated under reduced pressure to give the title compound (163 mg, 90% purity, 99% yield) as white solids. LC-MS (ESI): mass calcd. for C₁₃H₁₉ClF₂N₂O₂ 308.1, m/z found 273.4 [M−HCl+H]⁺.

T22: (S)-Ethyl 6-(((cis)-5-(1-((allyloxy)carbonyl)cyclopropyl)-3,3-difluorohexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate

To a mixture of T22-2 (163 mg, 90% purity, 0.476 mmol) in dichloromethane (5 mL) was added 2,2′,2″-nitrilotriethanol (710 mg, 95% purity, 4.76 mmol) and (S)-ethyl 6-(bromomethyl)-4-(3-fluoro-2-methylphenyl)-2-(thiazol-2-yl)-1,4-dihydropyrimidine-5-carboxylate H2-1A (312 mg, 0.714 mmol) successively at room temperature. After stirred at 40° C. for 16 hours under nitrogen atmosphere, The mixture was concentrated under reduced pressure. The resulting residue was purified by C18 column (acetonitrile:water=30% to 95%) to give the title compound (250 mg, 100% purity, 49% yield) as yellow solids. LC-MS (ESI): mass calcd. for C₃₁H₃₄F₃N₅O₄S 629.2, m/z found 630.4 [M+H]⁺.

### Compound 196: 1-((cis)-1-(((S)-5-(Ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)cyclopropanecarboxylic acid (Single Diastereomer)

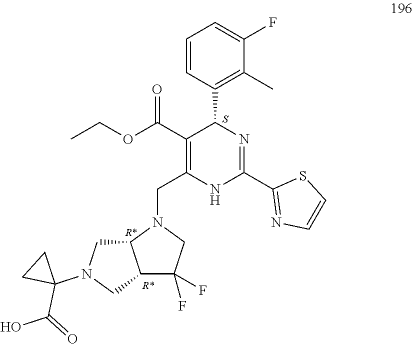

This compound was made from T22 according to typical method 2. Purified by HPLC (Column: Waters Xbrige C18 (5 μm 19*150 mm), Mobile phase A: water (0.1% trifluoroacetic acid), Mobile phase B: acetonitrile, UV: 214 nm, Flow rate: 15 mL/min, Gradient: 20-75% (% B)) to give the title compound (114.6 mg, 95.7% purity, 49.4% yield) as yellow solids. LC-MS (ESI): mass calcd. for C₂₈H₃₀F₃N₅O₄S 589.2, m/z found 590.2 [M+H]⁺. ¹H NMR (400 MHz, CDCl₃+ one drop of D₂O)¹H NMR (400 MHz, CDCl₃) δ 7.85 (s, 1H), 7.42 (d, J 3.2 Hz, 1H), 7.10-7.05 (m, 1H), 6.99 (d, J=2.8 Hz, 1H), 6.92-6.88 (m, 1H), 6.06 (s, 0.1H), 6.01 (s, 0.9H), 4.89 (d, J 14.0 Hz, 0.1H), 4.46 (d, J=16.4 Hz, 0.9H), 4.09-4.02 (m, 2H), 3.97-3.88 (m, 1H), 3.65-3.62 (m, 1H), 3.51-3.26 (m, 2H), 3.11-2.72 (m, 5H), 2.53 (s, 2.7H), 2.38 (s, 0.3H), 1.57-1.50 (m, 0.2H), 1.42-1.25 (m, 1.8H), 1.16-0.98 (m, 5H).

### Preparation of Intermediate T12

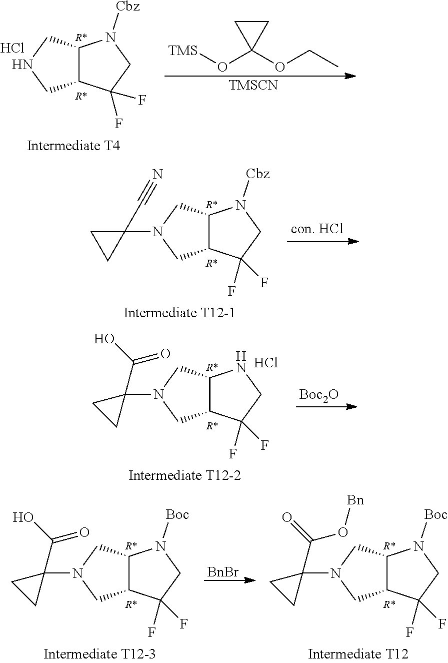

### T12-1: (cis)-Benzyl 5-(1-cyanocyclopropyl)-3,3-difluorohexahydropyrrolo[3,4-b]pyrrole-1(2H)-carboxylate

To the solution of T4 (600 mg, 90% purity, 1.70 mmol) in acetic acid (2 mL) was added (1-ethoxycyclopropoxy)trimethylsilane (450 mg, 2.58 mmol) and trimethylsilanecarbonitrile (750 mg, 7.56 mmol) under nitrogen atmosphere. After stirred at room temperature overnight, the reaction mixture was diluted with dichloromethane (20 mL) and basified with 32% sodium hydroxide aqueous solution to pH˜9. The resulting mixture was extracted with dichloromethane (20 mL) twice. The combined organic phases were dried over Na₂SO₄₍ₛ₎ and filtered. The filtrate was concentrated to give a residue, which was purified by C18 column (acetonitrile:water=5% to 95%) to give the title compound (620 mg, 90% purity from ¹H NMR, 95% yield) as brown oil. LC-MS (ESI): mass calcd. for C₁₈H₁₉F₂N₃O₂ 347.1, m/z found 348.1 [M+H]⁺. ¹H NMR (400 MHz, CDCl₃) δ 7.39-7.34 (m, 5H), 5.17-5.13 (m, 2H), 4.61-4.53 (m, 1H), 4.09-3.94 (m, 1H), 3.51-3.39 (m, 1H), 3.14-2.97 (m, 3H), 2.85-2.72 (m, 2H), 1.22-1.18 (m, 2H), 1.02-0.98 (m, 2H).

### T12-2: 1-((cis)-3,3-Difluorohexahydropyrrolo[3,4-b]pyrrol-5(1H)-yl)cyclopropanecarboxylic acid hydrochloride

The solution of T12-1 (620 mg, 90% purity, 1.61 mmol) in concentrated hydrochloride aqueous solution (8 mL) was stirred at 100° C. overnight. After cooled down to room temperature, the mixture was concentrated to give the title compound (600 mg, 56.2% purity, 78% yield) as brown oil. LC-MS (ESI): mass calcd. for C₁₀H₁₅ClF₂N₂O₂ 268.1, m/z found 233.1 [M−HCl+H]⁺.

### T12-3: 1-((cis)-1-(tert-Butoxycarbonyl)-3,3-difluorohexahydropyrrolo[3,4-b]pyrrol-5(1H)-yl)cyclopropanecarboxylic acid

To the solution of T12-2 (640 mg, 56.2% purity, 0.983 mmol) in tetrahydrofuran (10 mL) and water (1 mL) was added di-tert-butyl dicarbonate (420 mg, 1.92 mmol) and sodium hydroxide (300 mg, 7.50 mmol) at 0° C. After stirred at room temperature for 2 hours, the mixture was concentrated under reduced pressure to give a residue, which was acidified with 1 M hydrochloride aqueous solution to pH˜3 and extracted with ethyl acetate (40 mL) for three times. The combined organic layers were dried over Na₂SO₄₍ₛ₎ and filtered. The filtrate was concentrated to give the title compound (400 mg, 90% purity from ¹H NMR, 81% yield) as brown oil. LC-MS (ESI): mass calcd. for C₁₅H₂₂F₂N₂O₄ 332.2, m/z found 333.1 [M+H]⁺. ¹H NMR (400 MHz, CDCl₃) δ 4.47-4.36 (m, 1H), 4.00-3.80 (m, 1H), 3.49-3.40 (m, 1H), 3.22-2.92 (m, 5H), 1.46 (s, 9H), 1.39-1.33 (m, 2H), 1.06-0.98 (m, 2H).

### T12: (cis)-tert-Butyl 5-(1-((benzyloxy)carbonyl)cyclopropyl)-3,3-difluorohexahydropyrrolo[3,4-b]pyrrole-1(2H)-carboxylate

To a solution of T12-3 (400 mg, 90% purity, 1.08 mmol) in N,N-dimethylformamide (5 mL) was added benzyl bromide (205 mg, 1.20 mmol) and potassium carbonate (450 mg, 3.26 mmol). After stirred at room temperature overnight, the mixture was poured into water (50 mL), extracted with ethyl acetate (50 mL) twice. The combined organic layers were washed with brine (50 mL), dried over Na₂SO₄₍ₛ₎, filtered and concentrated to give a residue, which was purified by silica gel column chromatography (petroleum ether:ethyl acetate=20:1 to 10:1) to afford the title compound (310 mg, 90% purity from ¹H NMR, 61.0% yield) as colorless oil. LC-MS (ESI): mass calcd. for C₂₂H₂₈F₂N₂O₄ 422.2, m/z found 423.2 [M+H]⁺. ¹H NMR (400 MHz, CDCl₃) δ 7.36-7.28 (m, 5H), 5.13-5.05 (m, 2H), 4.44-4.32 (m, 1H), 3.96-3.79 (m, 1H), 3.40-2.87 (m, 6H), 1.46-1.45 (m, 9H), 1.38-1.33 (m, 2H), 0.97-0.88 (m, 2H).

### Compound 197 and compound 198: 1-((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)cyclopropane-1-carboxylic (Single Diastereomer)

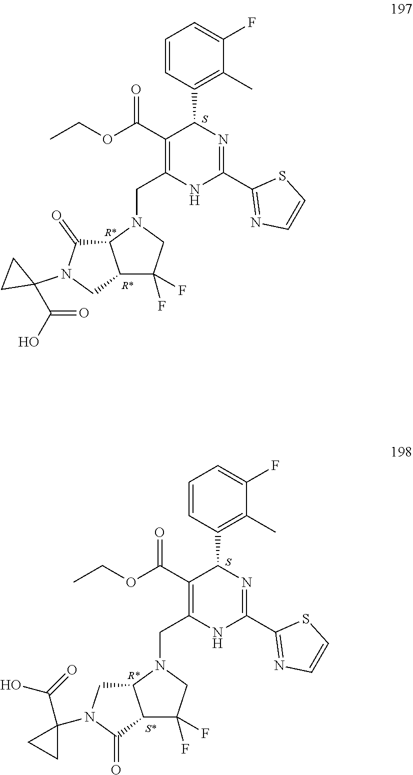

These two compounds were made using the procedure similar to Compound 42 by replacing T10-1 with T12 and replacing H5-1A with H2-1A.

197, LC-MS (ESI): mass calcd. for C₂₈H₂₈F₃N₅O₅S 603.2, m/z found 604.2 [M+H]⁺. ¹H NMR (400 MHz, CDCl₃) δ 8.44 (br s, 1H), 7.89 (d, J=2.8 Hz, 1H), 7.43 (d, J=2.8 Hz, 1H), 7.12-7.07 (m, 1H), 7.02-7.00 (m, 1H), 6.92-6.88 (m, 1H), 5.96 (s, 1H), 4.80 (d, J=15.2 Hz, 1H), 4.19 (d, J=15.6 Hz, 1H), 4.01 (q, J=7.2 Hz, 2H), 3.88-3.84 (m, 1H), 3.62-3.49 (m, 3H), 3.19-3.01 (m, 2H), 2.49 (s, 3H), 1.73-1.71 (m, 1H), 1.50-1.42 (m, 2H), 1.12-1.11 (m, 1H), 1.07 (t, J=7.2 Hz, 3H).

198, LC-MS (ESI): mass calcd. for C₂₈H₂₈F₃N₅O₅S 603.2, m/z found 604.3 [M+H]⁺. ¹H NMR (400 MHz, CDCl₃) δ 8.77 (br s, 1H), 7.85-7.84 (m, 1H), 7.43-7.42 (m, 1H), 7.11-7.06 (m, 1H), 6.99-6.97 (m, 1H), 6.93-6.88 (m, 1H), 6.00 (s, 1H), 4.66 (d, J=16.4 Hz, 1H), 4.01 (q, J=7.2 Hz, 2H), 3.80 (d, J=15.2 Hz, 1H), 3.69-3.67 (m, 2H), 3.51-3.39 (m, 3H), 2.86-2.79 (m, 1H), 2.50 (s, 3H), 1.71-1.68 (m, 1H), 1.50-1.41 (m, 2H), 1.20-1.18 (m, 1H), 1.05 (t, J=7.2 Hz, 3H).

### Compound 199: 1-(((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)methyl)cyclopentane-1-carboxylic acid (Single Diastereomer)

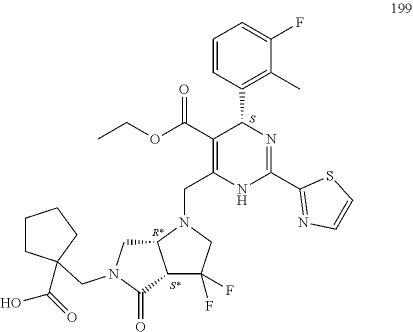

This compound was made using the procedure similar to Compound 42 by replacing tert-butyl 2,2-dimethyl-3-oxopropanoate with methyl 1-formylcyclopentane-1-carboxylate and replacing H5-1A with H2-1A. Purified by Prep.HPLC (Column: Waters Xbrige C18 (5 μm 10*190 mm), Mobile phase A: water (+0.1% ammonium bicarbonate), Mobile phase B: acetonitrile, UV: 214 nm, Flow rate: 15 mL/min, Gradient: 15-70% (% B)) to give the title compound (34 mg, 96.2% purity) as yellow solids. LC-MS (ESI): mass calcd. for C₃₁H₃₄F₃N₅O₅S 645.2, m/z found 646.2 [M+H]⁺. ¹H NMR (400 MHz, CD₃OD) δ 7.77 (d, J=3.2 Hz, 1H), 7.60 (d, J=3.2 Hz, 1H), 7.10-7.04 (m, 2H), 6.85-6.80 (m, 1H), 5.87 (s, 1H), 4.26 (d, J=16.8 Hz, 1H), 3.97-3.92 (m, 3H), 3.73-3.69 (m, 1H), 3.57 (d, J=14.0 Hz, 1H), 3.48-3.37 (m, 4H), 3.25-3.22 (m, 1H), 2.88-2.78 (m, 1H), 2.39 (d, J=2.4 Hz, 3H), 2.15-1.96 (m, 1H), 1.94-1.87 (m, 1H), 1.60-1.49 (m, 6H), 1.01 (t, J=11.2 Hz, 3H).

### Preparation of Intermediate T23

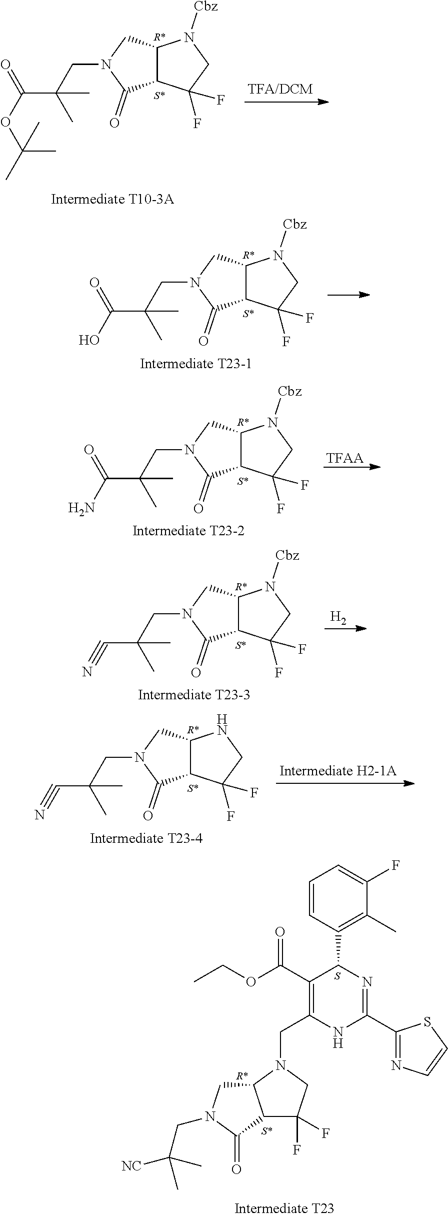

### T23-1: 3-((cis)-1-((benzyloxy)carbonyl)-3,3-difluoro-4-oxohexahydropyrrolo[3,4-b]pyrrol-5(1H)-yl)-2,2-dimethylpropanoic acid

To a solution of T10-3A (330 mg, 92% purity, 0.497 mmol) in dichloromethane (2 mL) was added trifluoroacetic acid (4 mL) at room temperature. After stirred at room temperature for 2 hours, the mixture was concentrated to give the desired product (260 mg, 66% purity, 65% yield) as white solids. LC-MS (ESI): mass calcd. for C₁₉H₂₂F₂N₂O₅ 396.2, m/z found 397.1 [M+H]⁺.

### T23-2: (cis)-benzyl 5-(3-amino-2,2-dimethyl-3-oxopropyl)-3,3-difluoro-4-oxohexahydropyrrolo[3,4-b]pyrrole-1(2H)-carboxylate

To a solution of T23-1 (260 mg, 66% purity, 0.433 mmol) in ethyl acetate (8 mL) was added 1,1′-carbonyldiimidazole (300 mg, 1.43 mmol). After stirred at room temperature for 2 hours, 28% ammonium hydroxide aqueous solution (1 mL) was added and the reaction was stirred at room temperature for 3 hours. Then the mixture was poured in water (30 mL) and extracted with ethyl acetate (15 mL) for three times. The combined ethyl acetate phases were washed with water (10 mL) for three times, dried over Na₂SO₄₍ₛ₎, filtered and the filtrate was concentrated to give a residue, which was purified by C18 column (acetonitrile:water=5% to 95%) to afford the title product (190 mg, 90% purity from ¹H NMR, 99% yield) as white solids. ¹H NMR (400 MHz, CDCl₃) δ 7.46-7.32 (m, 5H), 5.92-5.70 (m, 1H), 5.27-4.96 (m, 3H), 4.67-4.51 (m, 1H), 4.05-3.87 (m, 1H), 3.76-3.50 (m, 4H), 3.43-3.28 (m, 2H), 1.21-1.19 (m, 6H).

### T23-3: (cis)-benzyl 5-(2-cyano-2-methylpropyl)-3,3-difluoro-4-oxohexahydropyrrolo[3,4-b]pyrrole-1(2H)-carboxylate

To a solution of T23-2 (190 mg, 90% purity, 0.432 mmol) in dichloromethane (8 mL) was added trifluoroacetic anhydride (300 mg, 1.43 mmol) and pyridine (300 mg, 3.79 mmol). After stirred at room temperature for 2 hours under nitrogen atmosphere, the mixture was poured in water (30 mL) and extracted with ethyl acetate (15 mL) for three times. The combined ethyl acetate phases were washed with water (10 mL) for three times, dried over Na₂SO₄₍ₛ₎, filtered and the filtrate was concentrated. The residue was purified by C18 column chromatography (acetonitrile:water=5% to 95%) to afford the title product (140 mg, 90% purity from ¹H NMR, 77% yield) as white solids. ¹H NMR (400 MHz, CDCl₃) δ 7.36-7.32 (m, 5H), 5.17 (s, 2H), 4.78-4.65 (m, 1H), 4.05-3.87 (m, 3H), 3.82-3.69 (m, 1H), 3.63-3.54 (m, 1H), 3.48-3.39 (m, 1H), 3.28 (d, J=14.0 Hz, 1H), 1.37-1.35 (m, 6H).

### T23-4: 3-((cis)-3,3-difluoro-4-oxohexahydropyrrolo[3,4-b]pyrrol-5(1H)-yl)-2,2-dimethylpropanenitrile

To a solution of T23-3 (100 mg, 90% purity, 0.238 mmol) in ethyl acetate (5 mL) was added 10% palladium on carbon wt. (30 mg). The reaction was stirred at room temperature under a hydrogen balloon. After stirred at room temperature for 2 hours, the catalyst was filtered and washed with methanol (5 mL) and the filtrate was concentrated to afford the product (65 mg, 90% purity from ¹H NMR, 100% yield) as yellow oil. ¹H NMR (400 MHz, CDCl₃) δ 4.14 (t, J=6.8 Hz, 1H), 3.78-3.74 (m, 1H), 3.68-3.65 (m, 1H), 3.58 (d, J=14.0 Hz, 1H), 3.24-3.13 (m, 4H), 1.32-1.31 (m, 6H).

### T23: (S)-ethyl 6-(((cis)-5-(2-cyano-2-methylpropyl)-3,3-difluoro-4-oxohexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate

This compound was made from T23-4 and H2-1A according to typical method 1. ¹H NMR (400 MHz, CDCl₃) δ 9.17 (s, 1H), 7.72 (d, J=2.8 Hz, 1H), 7.39 (d, J=2.8 Hz, 1H), 7.12-7.03 (m, 2H), 6.93-6.87 (m, 1H), 6.02 (s, 1H), 4.47 (d, J=16.8 Hz, 1H), 4.11-3.96 (m, 4H), 3.81-3.72 (m, 1.5H), 3.71-3.67 (m, 1.5H), 3.51-3.37 (m, 2H), 3.23 (d, J=14.0 Hz, 1H), 2.91-2.80 (m, 1H), 2.52 (s, 3H), 1.44 (s, 3H), 1.38 (s, 3H), 1.11 (t, J=7.2 Hz, 3H).

### Compound 200: ethyl (S)-6-(((cis)-3,3-difluoro-5-(2-methyl-2-(2H-tetrazol-5-yl)propyl)-4-oxohexahydropyrrolo[3,4-b]pyrrol-1(2H)-yl)methyl)-4-(3-fluoro-2-methylphenyl)-2-(thiazol-2-yl)-1,4-dihydropyrimidine-5-carboxylate (Single Diastereomer)

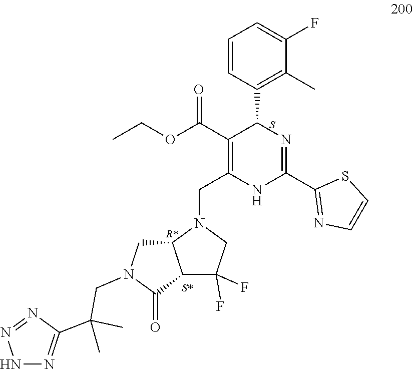

To a solution of T23 (90 mg, 90% purity, 0.14 mmol) in 1-methyl-2-pyrrolidinone (1.5 mL) was added azidotrimethylsilane (165 mg, 1.43 mmol) and dibutylstannanone (30 mg, 0.12 mmol) at room temperature under nitrogen atmosphere. After stirred at 140° C. for 10 hours under microwave, the mixture was concentrated under reduced pressure to afford the residue, which was purified by pre-HPLC (Column: Waters Xbrige C18 (5 μm 19*150 mm), Mobile phase A: water (0.2% formic acid), Mobile phase B: acetonitrile, UV: 214 nm, Flow rate: 15 mL/min, Gradient: 30-70% (% B)) to give the title compound (9.3 mg, 92.6% purity, 10% yield) as yellow solids. LC-MS (ESI): mass calcd. for C₂₉H₃₂F₃N₉O₃S 643.2, m/z found 644.3 [M+H]⁺. ¹H NMR (400 MHz, CD₃OD) δ 7.91 (d, J=3.2 Hz, 1H), 7.76 (d, J=3.2 Hz, 1H), 7.26-7.22 (m, 2H), 7.01-6.96 (m, 1H), 6.01 (s, 1H), 4.33 (d, J=16.0 Hz, 1H), 4.08 (q, J=7.2 Hz, 2H), 3.99 (d, J=16.4 Hz, 1H), 3.86 (d, J=13.6 Hz, 1H), 3.80 (t, J=6.8 Hz, 1H), 3.55-3.48 (m, 2H), 3.42-3.40 (m, 2H), 3.31-3.28 (m, 1H), 2.99-2.94 (m, 1H), 2.52 (d, J=2.4 Hz, 3H), 1.54 (s, 3H), 1.50 (s, 3H), 1.14 (t, J=7.2 Hz, 3H).

### Compound 201: 3-((cis)-1-((5-(ethoxycarbonyl)-6-(6-fluoro-2-methylpyridin-3-yl)-2-(thiazol-2-yl)-3,6-dihydropyrimidin-4-yl)methyl)-3,3-difluorohexahydropyrrolo[3,4-b]pyrrol-5(1H)-yl)-2,2-dimethylpropanoic acid (Single Diastereomer)

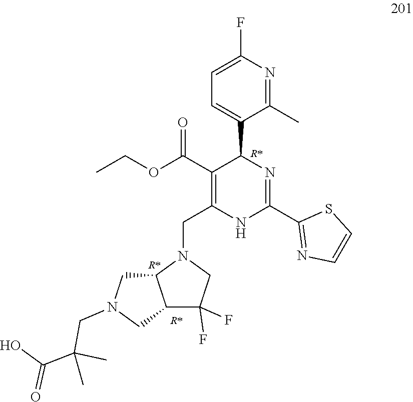 [SMILES: CCOC(=O)C1=C(CN2CC(F)(F)[C@@H]3CN(CC(C)(C)C(=O)O)C[C@@H]32)NC(c2nccs2)=N[C@@H]1c1ccc(F)nc1C]

This compound was made using the procedure similar to Compound 12 by replacing H2-1A with H20-1A. LC-MS (ESI): mass calcd. for C₂₈H₃₃F₃N₆O₄S 606.7, m/z found 607.3. ¹H NMR (400 MHz, CDCl₃) δ 9.21 (s, 1H), 7.90 (d, J=3.2 Hz, 1H), 7.59-7.55 (m, 1H), 7.43 (d, J=3.2 Hz, 1H), 6.71-6.68 (m, 1H), 5.98 (s, 1H), 4.39 (d, J=16.8 Hz, 1H), 4.09-3.98 (m, 3H), 3.73-3.70 (m, 1H), 3.57-3.50 (m, 1H), 3.39-3.30 (m, 2H), 3.08-3.02 (m, 1H), 2.94-2.86 (m, 1H), 2.78 (s, 3H), 2.74-2.65 (m, 2H), 2.57-2.51 (m, 2H), 1.28 (s, 3H), 1.24 (s, 3H), 1.13 (t, J=7.2 Hz, 3H).

### Compound 202: 2-(((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluoro-4-oxohexahydropyrrolo[3,4-b]pyrrol-5(1H)-yl)methyl)-2-ethylbutanoic acid (Single Diastereomer)

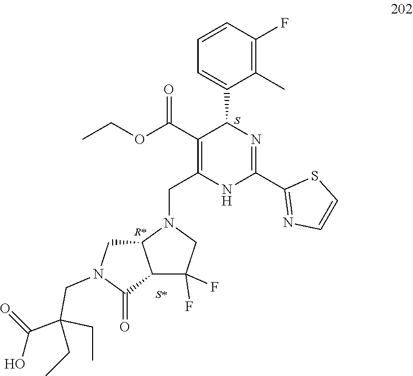

This compound was made using the procedure similar to Compound 42 by replacing tert-butyl 2,2-dimethyl-3-oxopropanoate with tert-butyl 2-ethyl-2-formylbutanoate and replacing H5-1A with H2-1A. Purified by Prep-HPLC (Column: Waters Xbrige C18 (5 μm 19*150 mm), Mobile phase A: water (0.1% ammonium bicarbonate), Mobile phase B: acetonitrile, UV: 254 nm, Flow rate: 15 mL/min, Gradient: 20-70% (% B)) to afford the desired product (15.0 mg, 96.6% purity) as a yellow solid. LC-MS (ESI): mass calcd. for C₃₁H₃₆F₃N₅O₅S 647.7, m/z found 648.3 [M+H]⁺. ¹H NMR (400 MHz, CDCl₃) δ 9.33 (s, 1H), 7.79 (d, J=3.2 Hz, 1H), 7.43 (d, J=3.6 Hz, 1H), 7.10-7.03 (m, 2H), 6.92 (t, J=8.4 Hz, 1H), 6.02 (s, 1H), 4.59 (d, J=14.8 Hz, 1H), 4.10-4.00 (m, 3H), 3.83 (t, J=10.4 Hz, 2H), 3.58-3.55 (m, 1H), 3.40-3.23 (m, 3H), 2.89 (d, J=14.0 Hz, 1H), 2.81-2.71 (m, 1H), 2.53 (s, 3H), 1.93-1.73 (m, 4H), 1.12 (t, J=7.2 Hz, 3H), 0.91-0.86 (m, 6H).

### Preparation of Intermediate S65

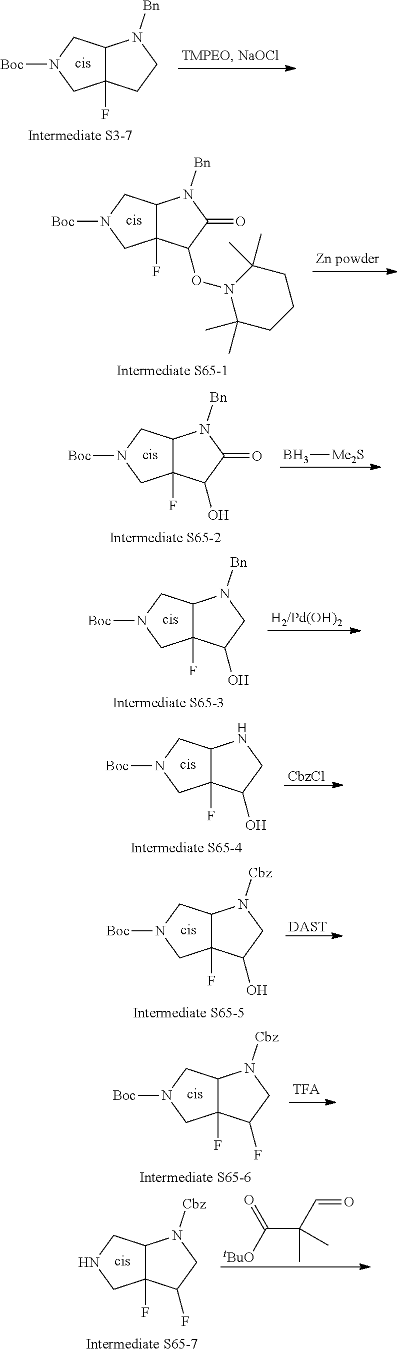

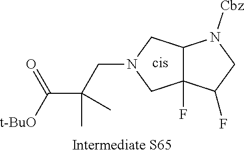 [SMILES: CC(C)(C)OC(=O)C(C)(C)CN1CC2N(C(=O)OCc3ccccc3)CC(F)C2(F)C1]

### S65-1: (cis)-tert-Butyl 1-benzyl-3a-fluoro-2-oxo-3-((2,2,6,6-tetramethylpiperidin-1-yl)oxy)hexahydropyrrolo[3,4-b]pyrrole-5(1H)-carboxylate

To a suspension of sodium phosphate monobasic (5.1 g, 42.5 mmol) in water (6 mL), sodium chlorite (770 mg, 8.51 mmol), and 2,2,6,6-tetramethylpiperidinooxy (1.00 g, 6.40 mmol) in acetonitrile (30 mL) was added 5.5% sodium hypochlorite solution (8 mL, 6.50 mmol) at 0° C., then S3-7 (1.5 g, 90% purity, 4.21 mmol) in acetonitrile (6 mL) was added. The mixture was stirred at 0° C. for 2 hours. Finally, saturated sodium hydroxide aqueous solution was added dropwise to quench the reaction until the red-wine color was turned into a clear-red color. The resulting phases were separated in a separatory funnel, then solids were washed with ethyl acetate (100 mL) three times. The combined organic phase was washed with brine (100 mL) twice, concentrated at vacuum and purified by C18 column (acetonitrile:water=25% to 95%) to give the title compound (1.4 g, 90% purity from ¹H NMR, 61% yield) as brown oil. LC-MS (ESI): mass calcd. for C₂H₄₀FN₃O₄ 489.3, m/z found 490.3 [M+H]⁺. ¹H NMR (400 MHz, CDCl₃) δ 7.36-7.18 (m, 5H), 5.16-4.56 (m, 2H), 4.21-3.10 (m, 6H), 1.50-1.17 (m, 27H).

### S65-2: (cis)-tert-Butyl 1-benzyl-3a-fluoro-3-hydroxy-2-oxohexahydropyrrolo[3,4-b]pyrrole-5(1H)-carboxylate

To a solution of S65-1 (3.5 g, 90% purity, 6.43 mmol) in acetic acid (50 mL), water (15 mL), and tetrahydrofuran (15 mL) was added zinc powder (17.0 g, 260 mmol). The suspension was allowed to react for 2 hours at 70° C. Then the solution was cooled down to room temperature and filtered. The filtrate was concentrated and purified by C18 column (acetonitrile:water=15% to 95%) to give the title compound (2.2 g, 90% purity from ¹H NMR, 88% yield) as colorless oil. LC-MS (ESI): mass calcd. for C₁₈H₂₃FN₂O₄ 350.2, m/z found 295.3 [M+H−56]⁺. ¹H NMR (400 MHz, CDCl₃) δ 7.37-7.19 (m, 5H), 5.09-5.05 (m, 0.2H), 4.83 (d, J=14.8 Hz, 0.8H), 4.73 (d, J=19.6 Hz, 0.3H), 4.34 (d, J=11.6 Hz, 0.7H), 4.19 (d, J=14.2 Hz, 0.7H), 4.02-3.85 (m, 2.3H), 3.65-3.46 (m, 3H), 3.33-3.29 (m, 1H), 1.43 (s, 9H).

### S65-3: (cis)-tert-Butyl 1-benzyl-3a-fluoro-3-hydroxyhexahydropyrrolo[3,4-b]pyrrole-5(1H)-carboxylate

To a solution of S65-2 (2.2 g, 90% purity, 5.65 mmol) in tetrahydrofuran (20 mL) was slowly added 10 M dimethylsulfide borane complex in dimethylsulfide (6 mL, 60.0 mmol) at 0° C. The reaction mixture was gradually warmed to room temperature and stirred for 3 hours. After stirred at room temperature overnight, the mixture was diluted with methanol (50 mL), concentrated and purified by C18 column (acetonitrile:water=5% to 95%) to give the title compound (1.9 g, 97.4% purity, 97% yield) as colorless oil. LC-MS (ESI): R_{T}=1.303 min, mass calcd. for C₁₈H₂₅FN₂O₃ 336.2, m/z found 337.2 [M+H]⁺.

### S65-4: (cis)-tert-Butyl 3a-fluoro-3-hydroxyhexahydropyrrolo[3,4-b]pyrrole-5(1H)-carboxylate

To a solution of S65-3 (500 mg, 97.4% purity, 1.45 mmol) in isopropyl alcohol (15 mL) was added 20% wt. palladium hydroxide on charcoal (140 mg, 0.199 mmol). The reaction mixture was stirred under hydrogen atmosphere (50 psi) overnight at 50° C. The reaction mixture was filtered. The filter cake was washed with isopropyl alcohol (20 mL) twice and concentrated in vacuo to give the title compound (630 mg, 55% purity, 97% yield) as colorless oil. LC-MS (ESI): mass calcd. for C₁₁H₁₉FN₂O₃ 246.1, m/z found 247.1 [M+H]⁺.

### S65-5: (cis)-1-Benzyl 5-tert-butyl 3a-fluoro-3-hydroxyhexahydropyrrolo[3,4-b]pyrrole-1,5-dicarboxylate

To a solution of S65-4 (630 mg, 55% purity, 1.40 mmol) in tetrahydrofuran (20 mL) was added benzyl carbonochloridate (0.3 mL, 2.10 mmol) and sodium bicarbonate (150 mg, 1.79 mmol) in water (4 mL) at room temperature. After stirred at room temperature for overnight, the mixture was poured into water (50 mL) and extracted with ethyl acetate (50 mL) for three times. The combined organic layers were washed with brine (100 mL), dried over Na₂SO₄₍ₛ₎, filtered and concentrated in vacuo to give a residue, which was purified by C18 column (acetonitrile:water=5% to 95%) to give the title compound (500 mg, 90% purity from ¹H NMR, 84% yield) as brown oil. LC-MS (ESI): mass calcd. for C₁₉H₂₅FN₂O₅ 380.2, m/z found 325.1 [M+H−56]⁺. ¹H NMR (400 MHz, CDCl₃) δ 7.36-7.32 (m, 5H), 5.14-5.12 (m, 2H), 4.55-4.25 (m, 2H), 3.97-3.40 (m, 6H), 2.44-2.37 (m, 1H), 1.44 (s, 9H).

### S65-6: (cis)-tert-Butyl 1-benzyl-3a-fluoro-3-hydroxy-2-oxohexahydropyrrolo[3,4-b]pyrrole-5(1H)-carboxylate

To a solution of S65-5 (350 mg, 90% purity, 0.828 mmol) in dichloromethane (10 mL) was added diethylaminosulfur trifluoride (1.2 mL) at 0° C. After stirred at 50° C. overnight, the mixture was diluted with ice water (50 mL), extracted with dichloromethane (50 mL) three times, washed with water (50 mL) and brine (50 mL), dried over Na₂SO₄₍ₛ₎ and filtered. The filtrate was concentrated and purified by C18 column (acetonitrile:water=from 5% to 95%) to give the title compound (170 mg, 90% purity form ¹H NMR, 48% yield) as brown oil. LC-MS (ESI): mass calcd. for C₁₉H₂₄F₂N₂O₄ 382.2, m/z found 327.1 [M+H−56]⁺. ¹H NMR (400 MHz, CDCl₃) δ 7.39-7.36 (m, 5H), 5.36-5.34 (m, 2H), 5.19-4.91 (m, 1H), 4.46-4.36 (m, 1H), 4.12-3.35 (m, 6H), 1.45 (s, 9H).

### S65-7: (cis)-Benzyl 3,3a-difluorohexahydropyrrolo[3,4-b]pyrrole-1(2H)-carboxylate

A solution of S65-6 (900 mg, 90% purity, 2.12 mmol) in dichloromethane (5 mL) and trifluoroacetic acid (5 mL) was stirred at room temperature for 1 hour. The mixture was poured into saturated sodium bicarbonate aqueous solution (10 mL), extracted with dichloromethane (20 mL) twice. The combined extracts were washed with brine (10 mL), concentrated to give the title compound (1.4 g, 58% purity, 99.3% yield) as brown oil. LC-MS (ESI): mass calcd. for C₁₄H₁₆F₂N₂O₂ 282.1, m/z found 283.1 [M+H]⁺.

### S65: (cis)-Benzyl 5-(3-(tert-butoxy)-2,2-dimethyl-3-oxopropyl)-3,3a-difluorohexahydropyrrolo[3,4-b]pyrrole-1(2H)-carboxylate

This intermediate was made from S65-7 and tert-butyl 2,2-dimethyl-3-oxopropanoate according to typical method 5. Purified by C18 column (acetonitrile:water=5% to 95%) to give the desired compound (870 mg, 90% purity from ¹H NMR, 87% yield) as brown oil. LC-MS (ESI): mass calcd. for C₂₃H₃₂F₂N₂O₄ 438.2, m/z found 439.2 [M+H]⁺. ¹H NMR (400 MHz, CDCl₃) δ 7.38-7.30 (m, 5H), 5.26-4.81 (m, 3H), 4.28-3.93 (m, 2H), 3.61-3.43 (m, 1.6H), 3.20-2.52 (m, 5.4H), 1.41-1.39 (m, 9H), 1.13-1.06 (m, 6H).

Chrial separation: chiral Prep. HPLC (method #1: Column: Chiralpak IC 5 μm 30*250 mm; Mobile Phase: CO₂:IPA=80:20 at 55 g/min; Temp: 30° C.; Wavelength: 214 nm; method #2: Column: Chiralpak IG 5 μm 20*250 mm; Mobile Phase: Hex:EtOH=75:25 at 15 mL/min; Temp: 30° C.; Wavelength: 214 nm).

S65A: LC-MS (ESI): mass calcd. for C₂₃H₃₂F₂N₂O₄ 438.2, m/z found 439.2 [M+H−56]⁺. Chiral analysis (Column: Chiralpak IG 5 μm 4.6*250 mm; Mobile Phase: Hex:EtOH=75:25 at 1 mL/min; Temp: 30° C.; Wavelength: 214 nm, R_{T}=5.399 min). ¹H NMR (400 MHz, CDCl₃) δ 7.35-7.32 (m, 5H), 5.35-5.20 (m, 0.6H), 5.16-5.06 (m, 2.4H), 4.25-4.08 (m, 2H), 3.59-3.43 (m, 2H), 3.03-2.93 (m, 1.5H), 2.83-2.80 (m, 0.5H), 2.75-2.54 (m, 3H), 1.41 (s, 4.5H), 1.40 (s, 4.5H), 1.11-1.10 (m, 6H).

S65B: LC-MS (ESI): mass calcd. for C₂₃H₃₂F₂N₂O₄ 438.2, m/z found 439.2 [M+H−56]⁺. Chiral analysis (Column: Chiralpak IG 5 μm 4.6*250 mm; Mobile Phase: Hex:EtOH=75:25 at 1 mL/min; Temp: 30° C.; Wavelength: 214 nm, R_{T}=8.862 min). ¹H NMR (400 MHz, CDCl₃) δ 7.35-7.32 (m, 5H), 5.35-5.21 (m, 0.6H), 5.16-5.05 (m, 2.4H), 4.25-4.08 (m, 2H), 3.59-3.43 (m, 2H), 3.03-2.93 (m, 1.5H), 2.83-2.80 (m, 0.5H), 2.75-2.54 (m, 3H), 1.41 (s, 4.5H), 1.40 (s, 4.5H), 1.11-1.10 (m, 6H).

S65C: LC-MS (ESI): mass calcd. for C₂₃H₃₂F₂N₂O₄ 438.2, m/z found 439.3 [M+H−56]⁺. Chiral analysis (Column: Chiralpak IC 5 μm 4.6*250 mm; Mobile Phase: CO₂:IPA=80:20 at 3 g/min; Temp: 30° C.; Wavelength: 214 nm, R_{T}=2.22 min). ¹H NMR (400 MHz, CDCl₃) δ 7.38-7.30 (m, 5H), 5.18-5.09 (m, 2H), 4.89 (dd, J=53.2, 6.0 Hz, 1H), 4.28-3.94 (m, 2H), 3.61-3.46 (m, 1H), 3.20-2.97 (m, 2H), 2.83-2.71 (m, 2H), 2.65-2.52 (m, 2H), 1.40-1.39 (m, 9H), 1.08-1.06 (m, 6H).

S65D: LC-MS (ESI): mass calcd. for C₂₃H₃₂F₂N₂O₄ 438.2, m/z found 439.3 [M+H−56]⁺. Chiral analysis (Column: Chiralpak IC 5 μm 4.6*250 mm; Mobile Phase: CO₂:IPA=80:20 at 3 g/min; Temp: 30° C.; Wavelength: 214 nm, R_{T}=2.70 min). ¹H NMR (400 MHz, CDCl₃) δ 7.38-7.30 (m, 5H), 5.18-5.10 (m, 2H), 4.89 (dd, J=53.2, 6.0 Hz, 1H), 4.28-3.94 (m, 2H), 3.61-3.46 (m, 1H), 3.20-2.97 (m, 2H), 2.83-2.71 (m, 2H), 2.65-2.52 (m, 2H), 1.40-1.39 (m, 9H), 1.08-1.06 (m, 6H).

### Preparation of Intermediate S66A

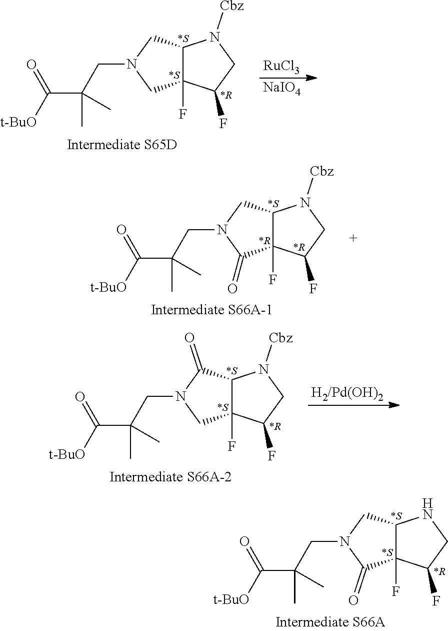

S66A-1/2:

To a solution of S65D (170 mg, 90% purity, 0.349 mmol) in ethyl acetate (2 mL) and water (2 mL) was added sodium periodate (190 mg, 0.759 mmol) and ruthenium(III) chloride hydrate (20 mg, 97% purity, 0.086 mmol) at room temperature. After stirred at room temperature for 20 minutes, the reaction mixture was diluted with water (40 mL) and extracted with ethyl acetate (40 mL) twice. The combined organic layers were washed with brine (40 mL), dried over Na₂SO₄₍ₛ₎, filtered and concentrated. The residue was purified by C18 column (acetonitrile:water=5% to 95%) and separated by chiral prep. HPLC 5 (Column: Chiralpak IC 5 μm 30*250 mm; Mobile Phase: Hex:EtOH=70:30 at 30 mL/min; Temp: 30° C.; Wavelength: 214 nm) to give the title compounds S66A-1 (60 mg, 90% purity from ¹H NMR, 34% yield) and S66A-2 (70 mg, 90% purity from ¹H NMR, 40% yield) as colorless oil.

S66A-1: LC-MS (ESI): mass calcd. for C₂₃H₃₀F₂N₂O₅ 452.2, m/z found 397.2 [M+H−56]⁺. Chiral analysis (Column: Chiralpak IC 5 μm 4.6*250 mm; Mobile Phase: Hex:EtOH=70:30 at 1 mL/min; Temp: 30° C.; Wavelength: 214 nm, R_{T}=5.179 min). ¹H NMR (400 MHz, CDCl₃) δ 7.40-7.33 (m, 5H), 5.36-5.27 (m, 0.6H), 5.20-5.11 (m, 2.4H), 4.47-4.37 (m, 1H), 4.20-3.97 (m, 1H), 3.76-3.69 (m, 1.5H), 3.55-3.35 (m, 3.5H), 1.45 (s, 5H), 1.38 (s, 4H), 1.16-1.10 (m, 6H).

S66A-2: LC-MS (ESI): mass calcd. for C₂₃H₃₀F₂N₂O₅ 452.2, m/z found 453.2 [M+H]f. Chiral analysis (Column: Chiralpak IC 5 μm 4.6*250 mm; Mobile Phase: Hex:EtOH=70:30 at 1 mL/min; Temp: 30° C.; Wavelength: 214 nm, R_{T}=6.907 min). ¹H NMR (400 MHz, CDCl₃) δ 7.45-7.30 (m, 5H), 5.21 (br s, 2H), 4.98-4.66 (m, 2H), 4.18-4.14 (m, 1H), 3.77-3.48 (m, 5H), 1.45 (s, 9H), 1.18 (s, 3H), 1.14 (s, 3H).

S66A:

To a solution of S66A-1 (60 mg, 90% purity, 0.119 mmol) in isopropyl alcohol (4 mL) was added 20% wt. palladium hydroxide on charcoal (15 mg, 0.021 mmol). After stirred under hydrogen atmosphere 2 hours at room temperature. The reaction mixture was filtered, washed with isopropyl alcohol (10 mL) twice and concentrated in vacuo to give the title compound (50 mg, 74% purity, 97.4% yield) as colorless oil. LC-MS (ESI): mass calcd. for C₁₅H₂₄F₂N₂O₃ 318.2, m/z found 319.2 [M+H]⁺.

### Compound 204D: 3-((cis)-1-(((S)-5-(ethoxycarbonyl)-6-(3-fluoro-2-methylphenyl)-2-(thiazol-2-yl)-3,6-dihydropyrimidin-4-yl)methyl)-3,3a-difluoro-4-oxohexahydropyrrolo[3,4-b]pyrrol-5(1H)-yl)-2,2-dimethylpropanoic acid

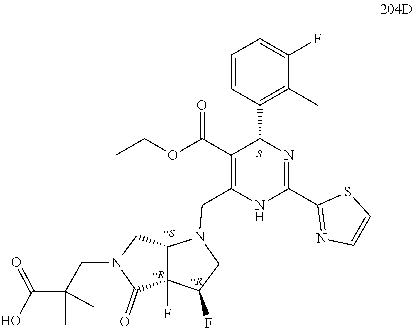

This compound was made from S66A and H2-1A according to typical method 1 and 3. LC-MS (ESI): mass calcd. for C₂₉H₃₂F₃N₅O₅S 619.2, m/z found 620.1 [M+H]⁺. ¹H NMR (400 MHz, CDCl₃) δ 9.13 (s, 1H), 7.82 (d, J=3.6 Hz, 1H), 7.46 (d, J=3.2 Hz, 1H), 7.12-7.06 (m, 1H), 7.01-6.99 (m, 1H), 6.94-6.90 (m, 1H), 6.02 (s, 1H), 5.12 (dt, J=53.6, 4.0 Hz, 1H), 4.66 (d, J=15.2 Hz, 1H), 4.10-3.99 (m, 4H), 3.80 (d, J=14.0 Hz, 1H), 3.66-3.58 (m, 2H), 3.46-3.36 (m, 1H), 3.02-2.91 (m, 2H), 2.53 (d, J=1.2 Hz, 3H), 1.36 (s, 3H), 1.35 (s, 3H), 1.13 (t, J=7.2 Hz, 3H).

### Compound 205A and 205B: 3-(((cis)-1-(((S)-5-(ethoxycarbonyl)-6-(3-fluoro-2-methylphenyl)-2-(thiazol-2-yl)-3,6-dihydropyrimidin-4-yl)methyl)-3,3-difluorohexahydropyrrolo[3,4-b]pyrrol-5(1H)-yl)sulfonyl)-1-methylcyclobutane-1-carboxylic acid

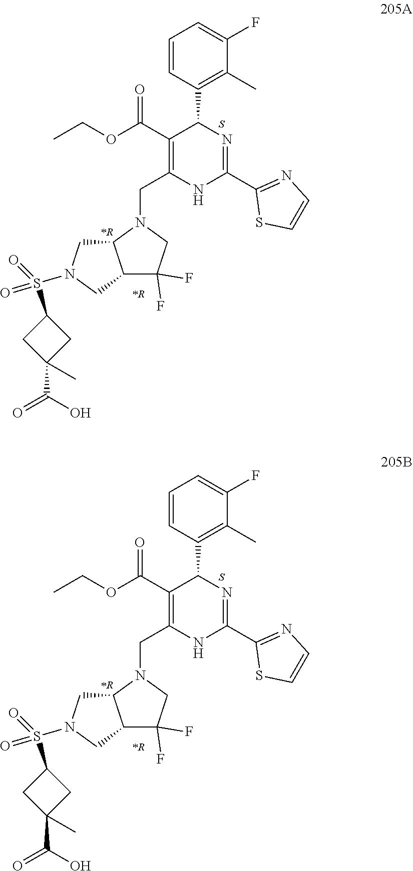

These 2 compounds were made using the procedure similar to Compound 46 by replacing tert-butyl 3-(chlorosulfonyl)-2,2-dimethylpropanoate with 2-(trimethylsilyl)ethyl (1r,3r)-3-(chlorosulfonyl)-1-methylcyclobutane-1-carboxylate. 205A, Purified by C18 column (MeCN:water containing 0.5% TFA=1% to 50%) to give title compound (55.5 mg) as a yellow solid. LCMS (ESI): mass calcd. For C₃₀H₃₄F₃N₅O₆S₂ 681.19, m/z found 682.2 [M+H]⁺. ¹H NMR (400 MHz, CD₃OD) δ=8.15 (d, J=4.0 Hz, 1H), 8.10 (d, J=4.0 Hz, 1H), 7.34-7.24 (m, 2H), 7.05 (m, 1H), 6.11 (s, 1H), 4.32-4.20 (m, 2H), 4.16-4.08 (m, 2H), 4.06-3.94 (m, 2H), 3.92-3.86 (m, 1H), 3.69 (d, J=12.0 Hz, 1H), 3.54 (m, 1H), 3.43-3.37 (m, 1H), 3.26-3.20 (m, 2H), 3.17-3.08 (m, 1H), 2.85-2.77 (m, 2H), 2.50 (d, J=2.0 Hz, 3H), 2.19-2.10 (m, 2H), 1.33 (s, 3H), 1.14 (t, J=8.0 Hz, 3H).

205B, LCMS (ESI): mass calcd. For C₃₀H₃₄F₃N₅O₆S₂ 681.19, m/z found 682.2 [M+H]⁺. ¹H NMR (400 MHz, CD₃OD) δ=8.09 (d, J=4.0 Hz, 1H), 7.99 (d, J=4.0 Hz, 1H), 7.28-7.19 (m, 2H), 7.02 (m, 1H), 6.07 (s, 1H), 4.25 (dd, J₁=16.0 Hz, J₂=20.0 Hz, 2H), 4.13-4.07 (m, 2H), 4.01 (t, J=8.0 Hz, 1H), 3.93-3.87 (m, 2H), 3.66 (d, J=12.0 Hz, 1H), 3.54-3.46 (m, 1H), 3.89-3.37 (m, 1H), 3.29-3.17 (m, 2H), 3.4-3.09 (m, 1H), 2.80-2.65 (m, 2H), 2.50 (d, J=2.1 Hz, 3H), 2.37-2.28 (m, 2H), 1.35 (s, 3H), 1.13 (t, J=8.0 Hz, 3H).

### Compound 206: 3-((cis)-1-((6-(2-chloro-3-fluorophenyl)-5-(methoxycarbonyl)-2-(thiazol-2-yl)-3,6-dihydropyrimidin-4-yl)methyl)-3,3-difluorohexahydropyrrolo[3,4-b]pyrrol-5(1H)-yl)-2,2-dimethyl-3-oxopropanoic acid (Single Diastereomer)

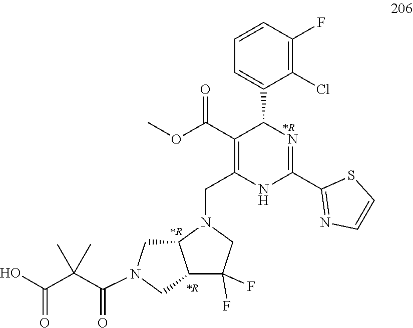 [SMILES: COC(=O)C1=C(CN2CC(F)(F)[C@@H]3CN(C(=O)C(C)(C)C(=O)O)C[C@@H]32)NC(c2nccs2)=N[C@H]1c1cccc(F)c1Cl]

This compound was made using the procedure similar to Compound 9 by replacing H2-1A with H11-1A. LCMS (ESI): mass calcd. For C₂₇H₂₇CF₃N₅O₅S 625.14, m/z found 626.2 [M+H]⁺. ¹H NMR (400 MHz, CDCl₃) δ: 8.84-8.57 (m, 1H), 8.15-7.71 (m, 1H), 7.58-7.42 (m, 1H), 7.25-6.96 (m, 3H), 6.31-6.19 (m, 1H), 4.82-4.32 (m, 1H), 4.32-4.11 (m, 1H), 4.03-3.81 (m, 1H), 3.80-3.44 (m, 5H), 3.44-2.94 (m, 4H), 2.89-2.58 (m, 1H), 1.41-1.15 (m, 6H).

### Compound 207: 3-((cis)-1-((6-(2-chloro-3-fluorophenyl)-5-(ethoxycarbonyl)-2-(thiazol-2-yl)-3,6-dihydropyrimidin-4-yl)methyl)-3,3-difluorohexahydropyrrolo[3,4-b]pyrrol-5(1H)-yl)-2,2-dimethyl-3-oxopropanoic acid (Single Diastereomer)

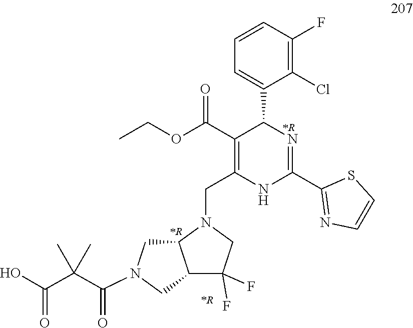 [SMILES: CCOC(=O)C1=C(CN2CC(F)(F)[C@@H]3CN(C(=O)C(C)(C)C(=O)O)C[C@@H]32)NC(c2nccs2)=N[C@H]1c1cccc(F)c1Cl]

This compound was made using the procedure similar to Compound 9 by replacing H2-1A with H1-1A. LCMS (ESI): mass calcd. For C₂₈H₂₉ClF₃N₅O₅S 639.15, m/z found 640.2 [M+H]⁺. ¹H NMR (400 MHz, CD₃OD) δ: 7.95-7.84 (m, 1H), 7.77-7.67 (m, 1H), 7.37-7.21 (m, 2H), 7.21-7.08 (m, 1H), 6.28-6.15 (m, 1H), 4.48-4.24 (m, 1H), 4.24-3.96 (m, 4H), 3.94-3.73 (m, 2H), 3.71-3.53 (m, 1H), 3.53-3.37 (m, 2H), 3.26-2.94 (m, 2H), 1.51-1.20 (m, 6H), 1.17-1.03 (m, 3H).

### Compound 208: 3-((cis)-1-((6-(2-chloro-4-fluorophenyl)-5-(methoxycarbonyl)-2-(thiazol-2-yl)-3,6-dihydropyrimidin-4-yl)methyl)-3,3-difluorohexahydropyrrolo[3,4-b]pyrrol-5(1H)-yl)-2,2-dimethyl-3-oxopropanoic acid (Single Diastereomer)

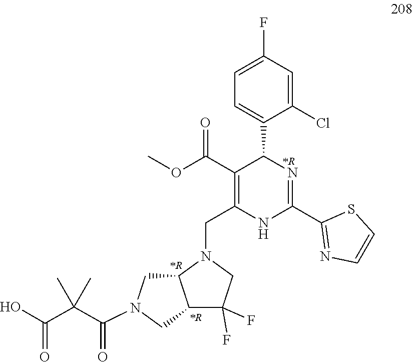 [SMILES: COC(=O)C1=C(CN2CC(F)(F)[C@@H]3CN(C(=O)C(C)(C)C(=O)O)C[C@@H]32)NC(c2nccs2)=N[C@H]1c1ccc(F)cc1Cl]

This compound was made using the procedure similar to Compound 9 by replacing H2-1A with H3-1A. LCMS (ESI): mass calcd. For C₂₇H₂₇ClF₃N₅O₅S 625.14, m/z found 626.2 [M+H]⁺. ¹H NMR (400 MHz, CD₃OD): δ: 7.97-7.83 (m, 1H), 7.78-7.69 (m, 1H), 7.51-7.37 (m, 1H), 7.29-7.18 (m, 1H), 7.14-6.99 (m, 1H), 6.24-6.09 (m, 1H), 4.47-4.22 (m, 1H), 4.22-3.74 (m, 4H), 3.70-3.56 (m, 4H), 3.55-3.35 (m, 2H), 3.26-2.95 (m, 2H), 1.51-1.19 (m, 6H).

### Compound 209A: 3-((cis)-1-((6-(2-chloro-3,4-difluorophenyl)-5-(methoxycarbonyl)-2-(thiazol-2-yl)-3,6-dihydropyrimidin-4-yl)methyl)-3a-fluoro-4-oxohexahydropyrrolo[3,4-b]pyrrol-5(1H)-yl)-2,2-dimethylpropanoic acid

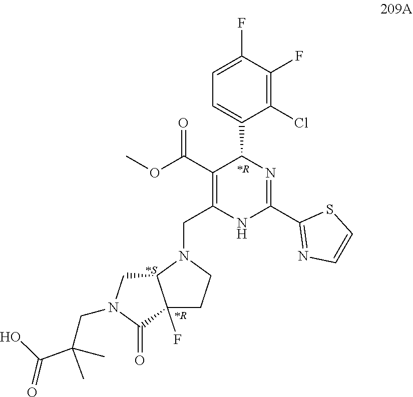 [SMILES: COC(=O)C1=C(CN2CC[C@]3(F)C(=O)N(CC(C)(C)C(=O)O)C[C@H]23)NC(c2nccs2)=N[C@H]1c1ccc(F)c(F)c1Cl]

This compound was made from H5-1A and S3A according to typical method 1. Purified by Prep. HPLC (Column: Xbridge C18 (5 μm 19*150 mm), Mobile Phase A: water (+0.1% ammonium bicarbonate), Mobile Phase B: acetonitrile, UV: 214 nm, Flow rate: 10 mL/min, Gradient: 15-70% (% B)) to give the title compounds (102.4 mg, 94.5% purity, 84% yield) as yellow solids. LC-MS (ESI): mass calcd. for C₂₇H₂₇CF₃N₅O₅S 625.14, m/z found 626.2 [M+H]⁺. ¹H NMR (400 MHz, CD₃OD) δ 7.78 (d, J=2.8 Hz, 1H), 7.63 (d, J=2.8 Hz, 1H), 7.16-7.12 (m, 2H), 6.07 (s, 1H), 4.29 (d, J=16.8 Hz, 2H), 3.99 (d, J=16.8 Hz, 1H), 3.65-3.60 (m, 1H), 3.55-3.51 (m, 1.5H), 3.51 (s, 3H), 3.48-3.45 (m, 0.5H), 3.40-3.35 (m, 2H), 3.00-2.96 (m, 1H), 2.79-2.73 (m, 1H), 2.28-2.18 (m, 2H), 1.12 (s, 3H), 1.09 (s, 3H).

### Preparation of Intermediate S52-A/B and 53-A/B

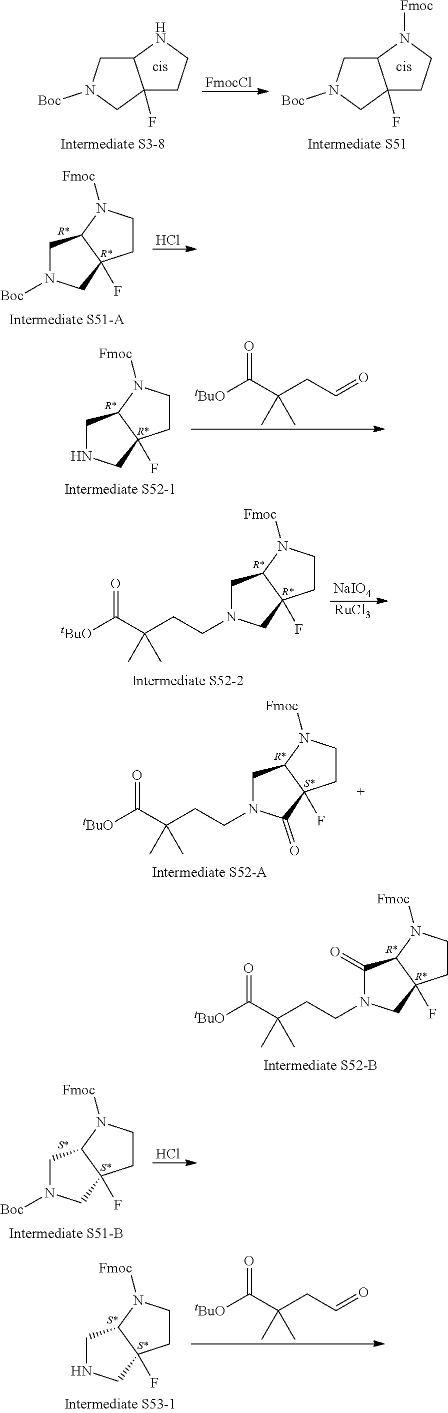

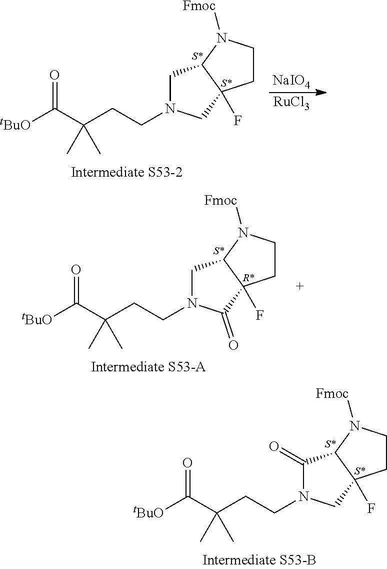

### S51: (cis)-1-((9H-Fluoren-9-yl)methyl) 5-tert-butyl 3a-fluorohexahydropyrrolo[3,4-b]pyrrole-1,5-dicarboxylate

To a solution of (cis)-tert-butyl 3a-fluorohexahydropyrrolo[3,4-b]pyrrole-5(1H)-carboxylate S3-8 (950 mg, 90% purity, 3.71 mmol) in 1,4-dioxane (8 mL) was added sodium carbonate (800 mg, 7.55 mmol) and 9-fluorenylmethyl chloroformate (1.15 g, 4.45 mmol) at 0° C. After stirred at room temperature overnight, the reaction mixture was quenched with water (10 mL), extracted with ethyl acetate (30 mL) for three times. The combined organic phases were dried over Na₂SO₄₍ₛ₎, filtered and concentrated under reduced pressure to give a residue, which was purified by silica gel column chromatography (petroleum ether:ethyl acetate=20:1 to 10:1) to give the title compound (934 mg, 90% purity from ¹H NMR, 50% yield) as yellow oil. ¹H NMR (400 MHz, CDCl₃) δ 7.77 (d, J=7.2 Hz, 2H), 7.57 (t, J=8.0 Hz, 2H), 7.41 (t, J=7.2 Hz, 2H), 7.32 (t, J=7.6 Hz, 2H), 4.67-4.37 (m, 2H), 4.34-3.96 (m, 2H), 3.90-3.09 (m, 6H), 2.42-2.03 (m, 2H), 1.46 (s, 9H).

Racemic S51 (934 mg, 90% purity, 1.86 mmol) was separated by chiral Prep. HPLC (Colum: Chiralpak IB 5 μm 20*250 mm; Mobile Phase: Hex:EtOH=75:25 at 30 mL/min; Temp: 30° C.; Wavelength: 254 nm) to afford the title compounds S51-A (440 mg, 90% purity from ¹H NMR, 47% yield, 100% stereopure) and S51-B (400 mg, 90% purity from ¹H NMR, 43% yield, 99% stereopure) as yellow oil.

S51-A: Chiral analysis (Column: Chiralpak IB 5 μm 4.6*250 mm; Mobile Phase: Hex:EtOH=75:25 at 30 mL/min; Temp: 30° C.; Wavelength: 254 nm, R_{T}=7.218 min). ¹H NMR (400 MHz, CDCl₃) δ 7.77 (d, J=7.6 Hz, 2H), 7.57 (t, J=8.0 Hz, 2H), 7.41 (t, J=7.6 Hz, 2H), 7.32 (t, J=7.2 Hz, 2H), 4.57-4.34 (m, 2H), 4.25-4.24 (m, 2H), 4.02-3.58 (m, 5H), 3.48-3.11 (m, 1H), 2.40-2.07 (m, 2H), 1.46 (s, 9H).

S51-B: Chiral analysis (Column: Chiralpak IB 5 μm 4.6*250 mm; Mobile Phase: Hex:EtOH=75:25 at 30 mL/min; Temp: 30° C.; Wavelength: 254 nm, R_{T}=9.238 min). ¹H NMR (400 MHz, CDCl₃) δ 7.77 (d, J=7.6 Hz, 2H), 7.57 (t, J=8.0 Hz, 2H), 7.41 (t, J=7.2 Hz, 2H), 7.32 (t, J=7.6 Hz, 2H), 4.54-4.41 (m, 2H), 4.32-4.22 (m, 2H), 4.02-3.37 (m, 6H), 2.38-2.04 (m, 2H), 1.46 (s, 9H).

### S52-1: (cis)-(9H-Fluoren-9-yl)methyl 3a-fluorohexahydropyrrolo[3,4-b]pyrrole-1(2H)-carboxylate hydrochloride

A mixture of S51-A (440 mg, 90% purity, 0.875 mmol) in 4 M hydrochloride in ethyl acetate (5 mL, 20 mmol) was stirred at 20° C. for 2 hours. The mixture was concentrated to give the title compound (380 mg, 90% purity from ¹H NMR, crude) as white solids. ¹H NMR (400 MHz, CDCl₃) δ 10.11 (br s, 1H), 7.77-7.75 (m, 2H), 7.60-7.53 (m, 2H), 7.42-7.30 (m, 4H), 4.66-4.22 (m, 4H), 3.89-3.62 (m, 5H), 3.15-3.07 (m, 0.5H), 2.88-2.82 (m, 0.5H), 2.51-2.25 (m, 2H)

### S52-2: (cis)-(9H-Fluoren-9-yl)methyl 5-(4-(tert-butoxy)-3,3-dimethyl-4-oxobutyl)-3a-fluorohexahydropyrrolo[3,4-b]pyrrole-1(2H)-carboxylate

Typical method 5: A mixture of S52-1 (380 mg, 90% purity, 0.879 mmol) and triethylamine (90 mg, 0.89 mmol) in dichloromethane (5 mL) was stirred at 25° C. for 0.5 hour, then tert-butyl 2,2-dimethyl-4-oxobutanoate (327 mg, 90% purity, 1.58 mmol) and 1 M triisopropoxytitanium(IV) chloride in dichloromethane (1.7 mL, 1.7 mmol) was added into the mixture, and the mixture was stirred at 25° C. for 0.5 hour. Acetic acid (0.5 mL) and sodium triacetoxyborohydride (500 mg, 2.34 mmol) were added into the mixture. After stirred at 25° C. for 14 hours, the mixture was diluted with water (50 mL), extracted with dichloromethane (100 mL) twice. The combined organic layers were washed with water (50 mL) for three times, brine (50 mL), dried with Na₂SO₄₍ₛ₎, filtered and concentrated under reduced pressure to give a residue, which was purified by C18 (acetonitrile:water=60% to 70%) to give the title compound (350 mg, 90% purity from ¹H NMR, 69% yield) as colorless oil. ¹H NMR (400 MHz, CDCl₃) δ 7.77 (d, J=7.6 Hz, 2H), 7.60-7.55 (m, 2H), 7.40 (t, J=7.6 Hz, 2H), 7.32 (t, J=7.6 Hz, 2H), 4.62-4.54 (m, 1H), 4.44-4.40 (m, 1H), 4.27-4.28 (m, 1H), 3.87-3.68 (m, 1H), 3.61-3.38 (m, 1H), 2.94-2.68 (m, 3H), 2.49-2.08 (m, 6H), 1.72-1.65 (m, 2H), 1.44 (s, 9H), 1.15 (s, 6H).

### S52-A/B: (cis)-(9H-Fluoren-9-yl)methyl 5-(4-(tert-butoxy)-3,3-dimethyl-4-oxobutyl)-3a-fluoro-4-oxohexahydropyrrolo[3,4-b]pyrrole-1(2H)-carboxylate and (cis)-(9H-fluoren-9-yl)methyl 5-(4-(tert-butoxy)-3,3-dimethyl-4-oxobutyl)-3a-fluoro-6-oxohexahydropyrrolo[3,4-b]pyrrole-1(2H)-carboxylate

These two compound were made from S52-2 analogous to S3-12. Purified by C18 (acetonitrile:water=10% to 55%) to give a yellow oil, which was separated by chiral Prep. HPLC (Column: Chiralpak IG 5 μm 30*250 mm; Mobile Phase: Hexane:EtOH=40:60 at 15 mL/min; Temp: 30° C.; Wavelength: 214 nm) to afford the title compounds S52-A (55 mg, 90% purity from ¹H NMR, 15% yield) and S52-B (60 mg, 90% purity from ¹H NMR, 17% yield) as yellow oil.

S52-A: ¹H NMR (400 MHz, CDCl₃) δ 7.81-7.76 (m, 2H), 7.56 (br s, 2H), 7.44-7.31 (m, 4H), 4.83-4.80 (m, 0.5H), 4.70-4.66 (m, 0.5H), 4.48-4.40 (m, 1H), 4.25-4.20 (m, 1H), 3.81-3.74 (m, 2H), 3.62-3.40 (m, 1.2H), 3.32-3.28 (m, 1.2H), 2.81-2.77 (m, 0.6H), 2.39-2.17 (m, 3H), 1.75-1.64 (m, 3H), 1.48-1.45 (m. 9H), 1.20-1.17 (m, 6H).

S52-B: ¹H NMR (400 MHz, CDCl₃) δ 7.76 (d, J=7.2 Hz, 2H), 7.60 (br s, 2H), 7.39 (t, J=7.2 Hz, 2H), 7.31 (t, J=7.2 Hz, 2H), 4.76 (d, J=19.6 Hz, 1H), 4.52-4.27 (m, 3H), 3.91-3.73 (m, 2H), 3.64-3.58 (m, 1H), 3.52-3.38 (m, 2H), 3.01-3.17 (m, 1H), 2.59-2.48 (m, 1H), 2.23-2.11 (m, 1H), 1.79-1.66 (m, 2H), 1.46 (s, 9H), 1.19 (s, 6H).

Analogously, intermediates 53-A and 53-B were prepared.

53-A: ¹H NMR (400 MHz, CDCl₃) δ 7.76-7.68 (m, 3H), 7.60 (br s, 1H), 7.39 (t, J=7.2 Hz, 2H), 7.31 (t, J=7.2 Hz, 2H), 4.78-4.74 (m, 1H), 4.53-4.27 (m, 3H), 3.84-3.76 (m, 2H), 3.64-3.59 (m, 1H), 3.52-3.38 (m, 2H), 3.24-3.19 (m, 1H), 2.57-2.46 (m, 1H), 2.45-2.11 (m, 1H), 1.72-1.61 (m, 2H), 1.46 (s, 9H), 1.19 (s, 6H).

53-B: ¹H NMR (400 MHz, CDCl₃) δ 7.80-7.76 (m, 2H), 7.56 (br s, 2H), 7.43-7.37 (m, 2H), 7.35-7.32 (m, 2H), 4.84-4.80 (m, 1H), 4.48-4.44 (m, 1H), 4.28 (br s, 1H), 3.86-3.62 (m, 2H), 3.49-3.38 (m, 1H), 3.30-3.22 (m, 1H), 3.14-3.10 (m, 1H), 2.46-2.12 (m, 3H), 1.75-1.52 (m, 3H), 1.48-1.41 (m, 9H), 1.20-1.17 (m, 6H).

### Compound 210B: 4-((cis)-1-(((S)-5-(ethoxycarbonyl)-6-(3-fluoro-2-methylphenyl)-2-(thiazol-2-yl)-3,6-dihydropyrimidin-4-yl)methyl)-3a-fluoro-4-oxohexahydropyrrolo[3,4-b]pyrrol-5(1H)-yl)-2,2-dimethylbutanoic acid

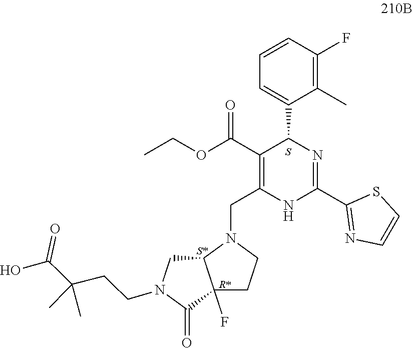

Compound 210B was made from H2-1A and S53-A according to typical method 1 and 3 successively. LC-MS (ESI): mass calcd. for C₃₀H₃₅F₂N₅O₅S 615.7, m/z found 616.2 [M+H]⁺. ¹H NMR (400 MHz, CD₃OD) δ 7.90 (d, J=3.2 Hz, 1H), 7.72 (d, J=3.2 Hz, 1H), 7.17-7.09 (m, 2H), 6.95-6.91 (m, 1H), 5.96 (s, 1H), 4.32-4.23 (m, 2H), 4.07 (q, J=7.6 Hz, 2H), 3.71-3.56 (m, 2H), 3.40-3.35 (m, 2H), 3.25-3.21 (m, 2H), 3.02-2.96 (m, 1H), 2.51 (s, 3H), 2.41-2.25 (m, 2H), 1.74-1.71 (m, 2H), 1.16-1.12 (m, 9H).

### Compound 211: 3-((cis)-1-((6-(4-chloro-3-fluorophenyl)-5-(ethoxycarbonyl)-2-(thiazol-2-yl)-3,6-dihydropyrimidin-4-yl)methyl)-3,3-difluoro-4-oxohexahydropyrrolo[3,4-b]pyrrol-5(1H)-yl)-2,2-dimethylpropanoic acid (Single Diastereomer)

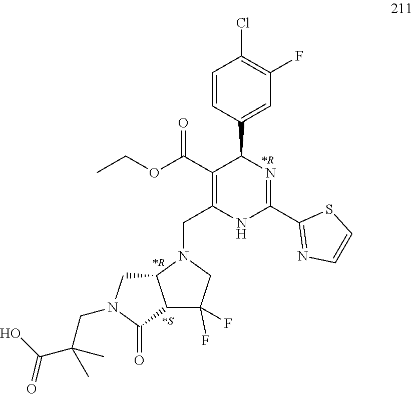

This compound was made using the procedure similar to Compound 42 by replacing H5-1A with H29-1A. Purified by flash column chromatography (Column: C18, 20-35 μm, 100A, 40 g) eluting with 5-50% Acetonitrile in water (add 0.05% HCOOH). 17 mg of product was obtained as yellow solid. ¹H NMR (400 MHz, CD₃OD) δ ppm 7.97 (d, J=3.06 Hz, 1H), 7.87 (d, J=3.06 Hz, 1H), 7.47 (t, J=7.89 Hz, 1H), 7.21-7.29 (m, 2H), 5.76 (s, 1H), 4.34 (d, J=16.51 Hz, 1H), 4.09-4.21 (m, 2H), 4.01 (d, J=16.63 Hz, 1H), 3.77-3.83 (m, 1H), 3.58-3.68 (m, 2H), 3.48-3.57 (m, 2H), 3.34-3.39 (m, 2H), 2.88-3.02 (m, 1H), 1.16-1.26 (m, 9H).

The following compounds were made according to the synthetic procedures described hereinabove, or analogous synthetic procedures:

TABLE 1

| | |
|---|---|
| 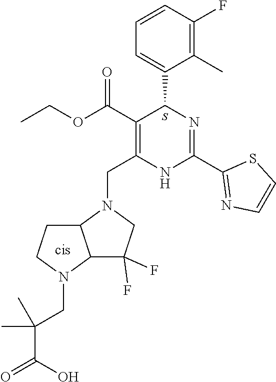 | 1 |
| 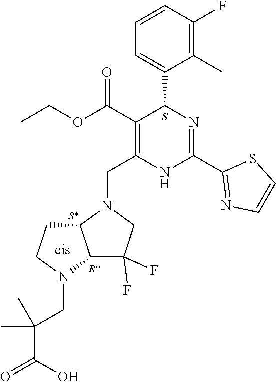 | 1A |
| 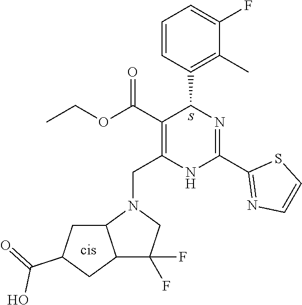 | 2 |
| 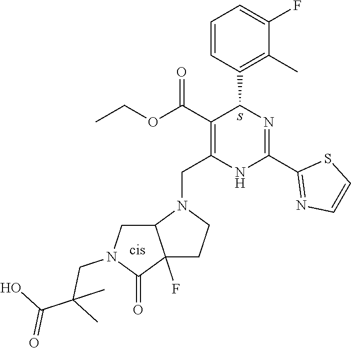 | 3 |
| 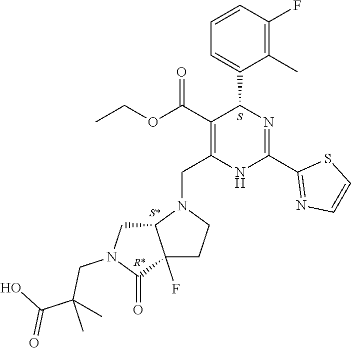 | 3B |
| 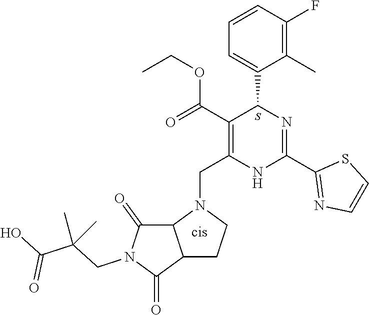 | 4 |
| 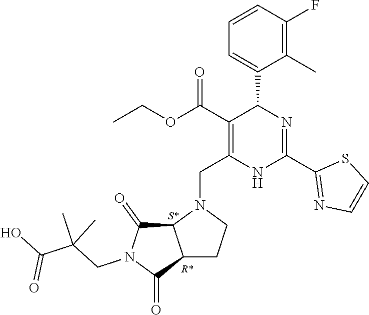 [SMILES: CCOC(=O)C1=C(CN2CC[C@H]3C(=O)N(CC(C)(C)C(=O)O)C(=O)[C@H]32)NC(c2nccs2)=N[C@H]1c1cccc(F)c1C] | 4A |
| 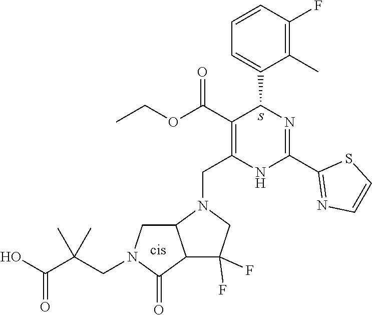 | 5 |
| 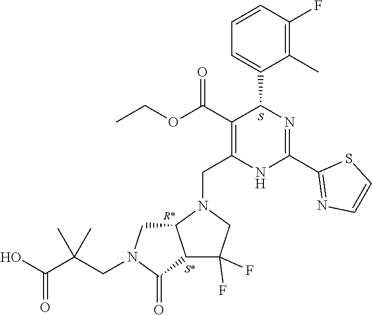 | 5A |
| 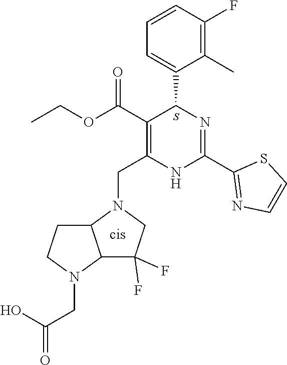 | 6 |
| 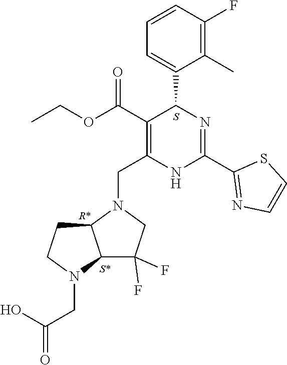 | 6A |
| 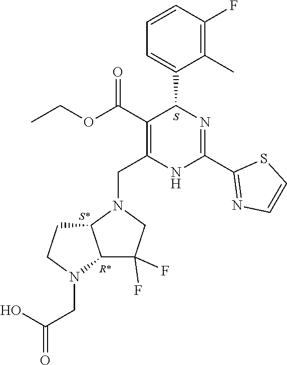 | 6B |
| 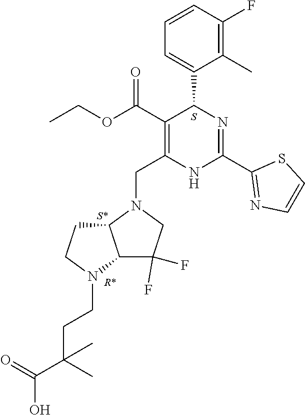 | 7A |
| 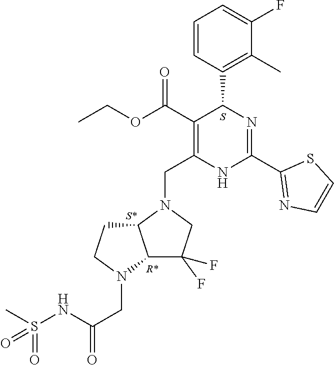 | 8A |
| 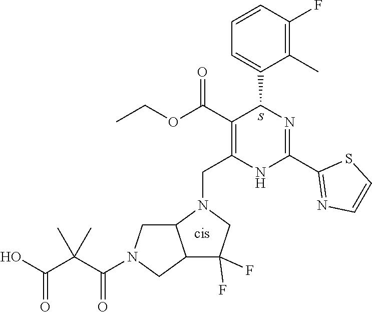 | 9 |
| 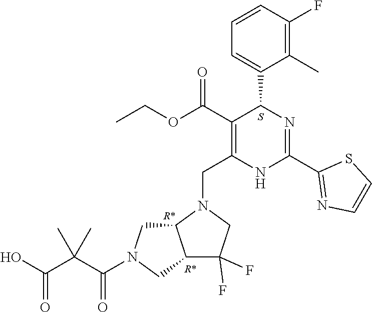 | 9A |
| 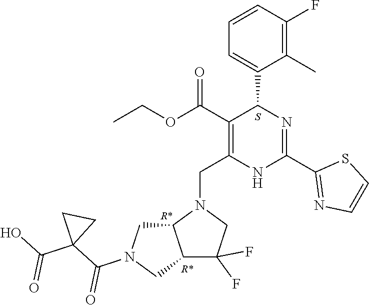 | 10 |
| 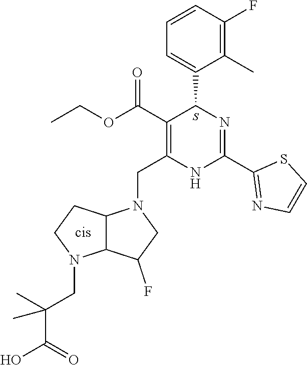 | 11 |
| 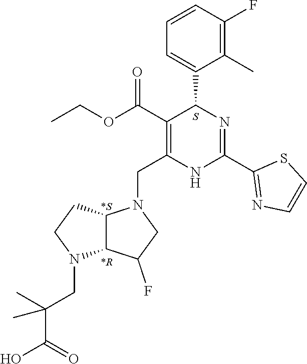 | 11B |
| 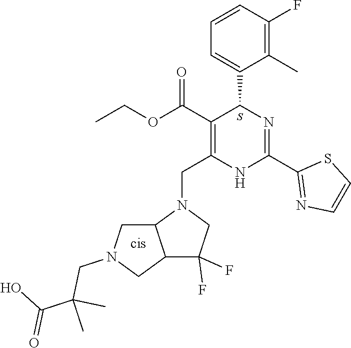 | 12 |
| 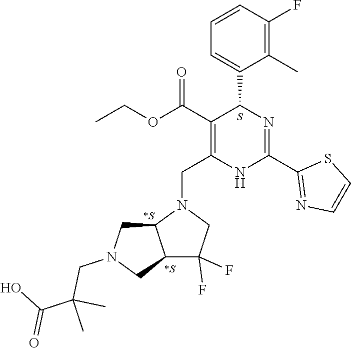 | 12B |
| 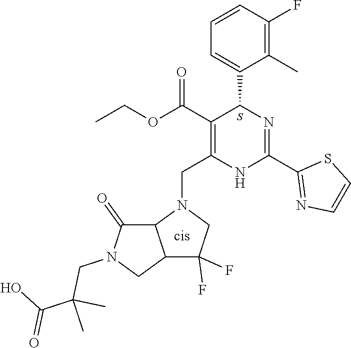 | 13 |
| 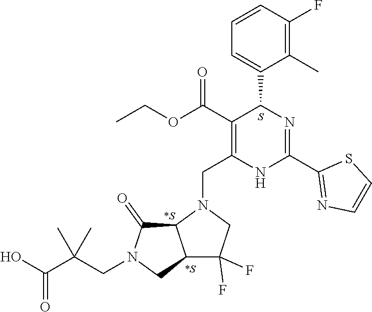 | 13B |
| 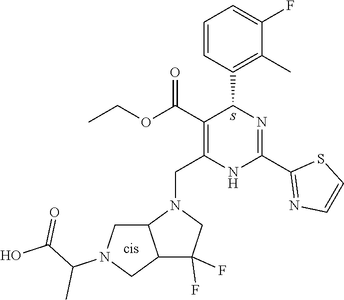 | 14 |
| 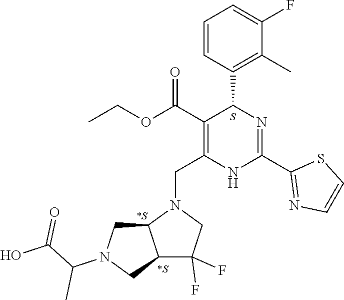 | 14B |
| 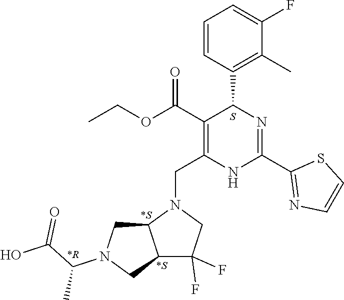 | 14E |
| 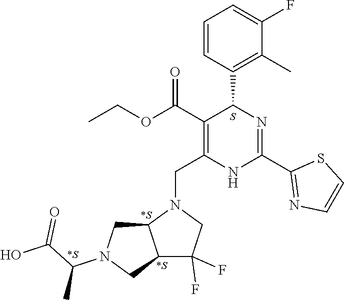 | 14F |
| 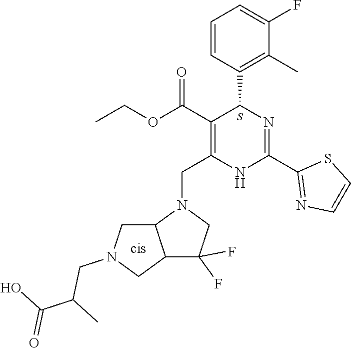 | 15 |
| 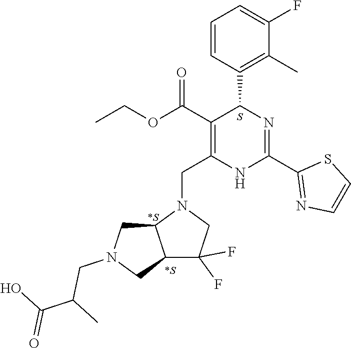 | 15B |
| 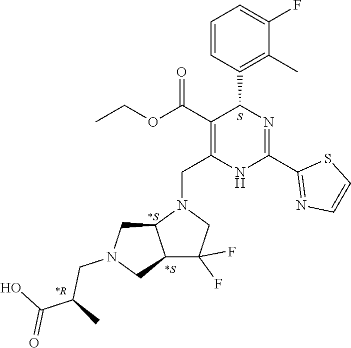 | 15E |
| 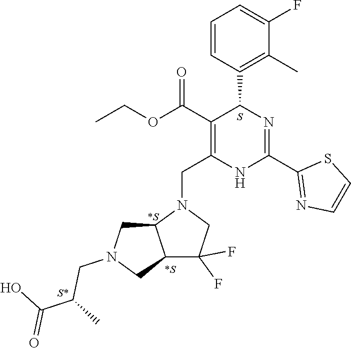 | 15F |
| 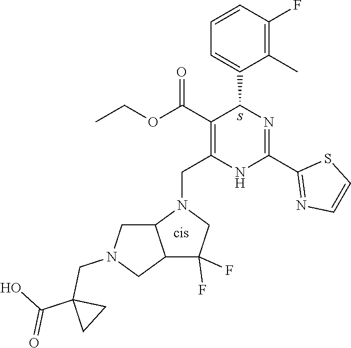 | 16 |
| 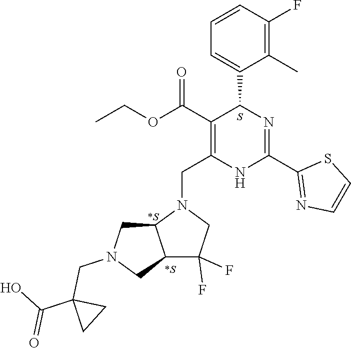 | 16B |
| 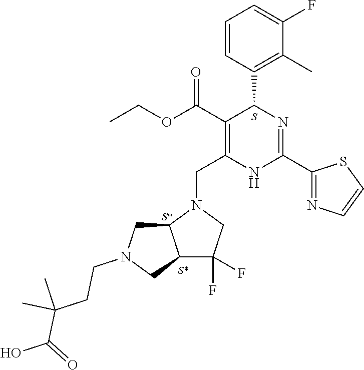 | 17B |
| 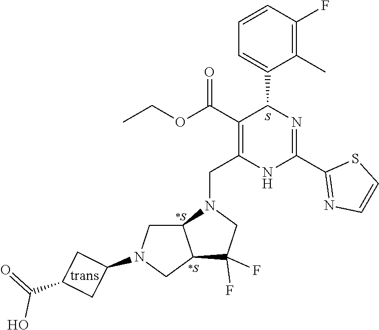 | 18A |
| 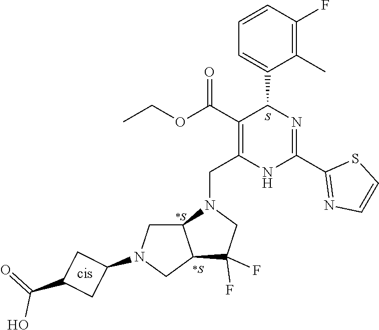 | 18B |
| 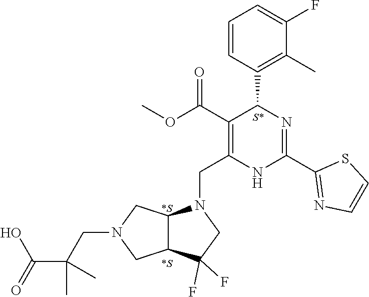 | 19B |
| 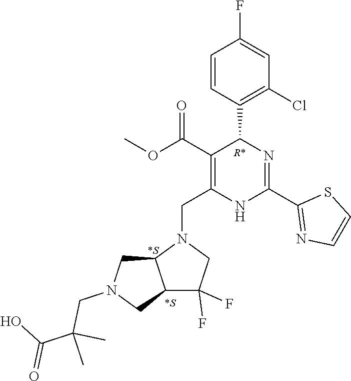 [SMILES: COC(=O)C1=C(CN2CC(F)(F)[C@H]3CN(CC(C)(C)C(=O)O)C[C@H]32)NC(c2nccs2)=N[C@H]1c1ccc(F)cc1Cl] | 20B |
| 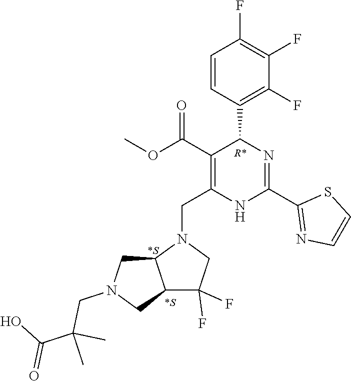 [SMILES: COC(=O)C1=C(CN2CC(F)(F)[C@H]3CN(CC(C)(C)C(=O)O)C[C@H]32)NC(c2nccs2)=N[C@H]1c1ccc(F)c(F)c1F] | 21B |
| 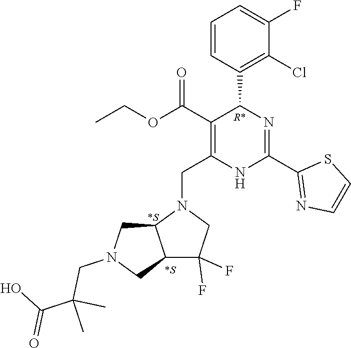 [SMILES: CCOC(=O)C1=C(CN2CC(F)(F)[C@H]3CN(CC(C)(C)C(=O)O)C[C@H]32)NC(c2nccs2)=N[C@H]1c1cccc(F)c1Cl] | 22B |
| 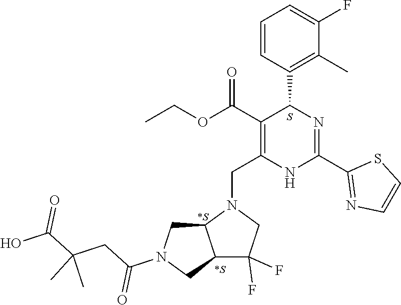 | 23B |
| 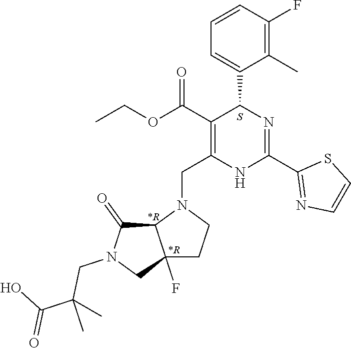 | 24A |
| 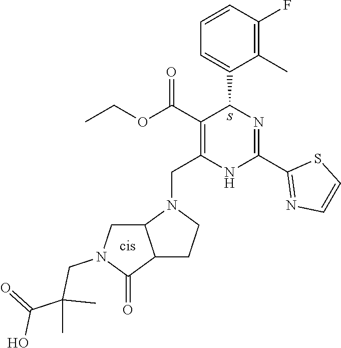 | 25 |
| 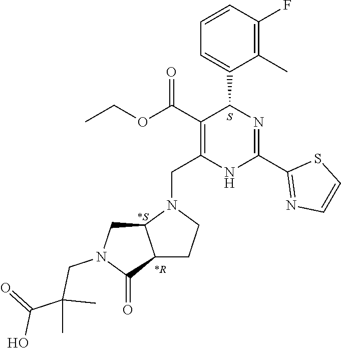 | 25A |
| 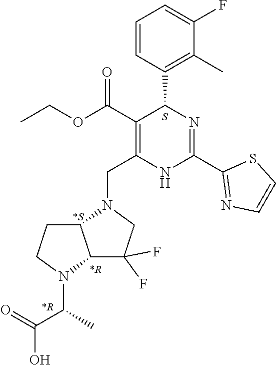 | 26E |
| 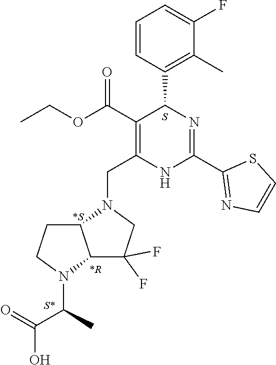 | 26F |
| 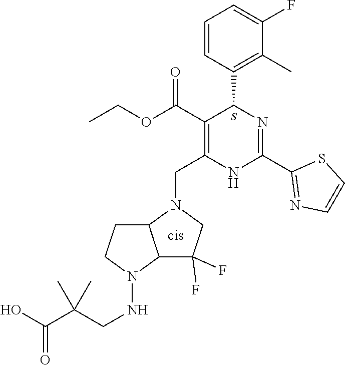 | 27 |
| 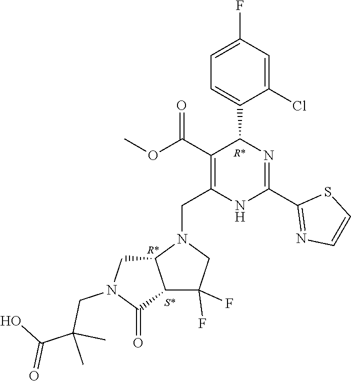 [SMILES: COC(=O)C1=C(CN2CC(F)(F)[C@@H]3C(=O)N(CC(C)(C)C(=O)O)C[C@@H]32)NC(c2nccs2)=N[C@H]1c1ccc(F)cc1Cl] | 28 |
| 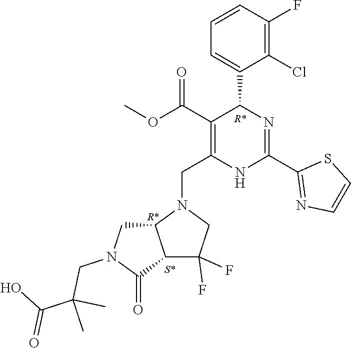 [SMILES: COC(=O)C1=C(CN2CC(F)(F)[C@@H]3C(=O)N(CC(C)(C)C(=O)O)C[C@@H]32)NC(c2nccs2)=N[C@H]1c1cccc(F)c1Cl] | 29 |
| 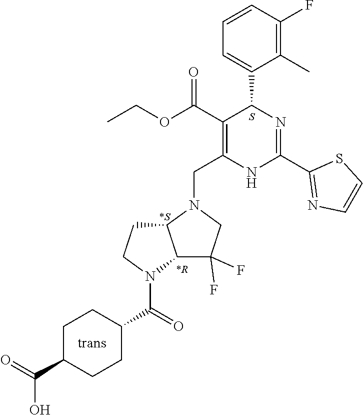 | 30 |
| 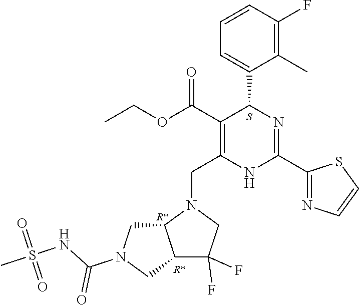 | 31 |
| 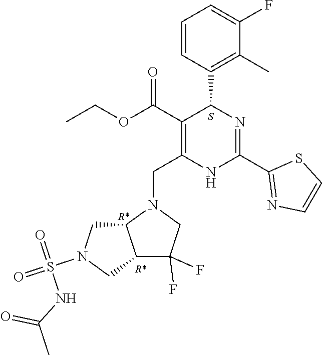 | 32 |
| 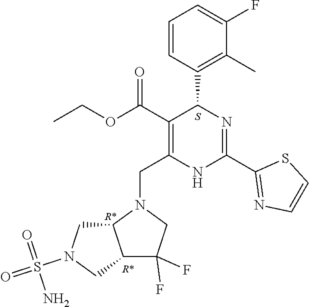 | 33 |
| 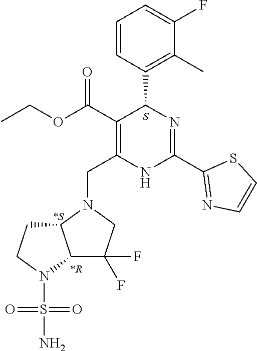 | 34 |
| 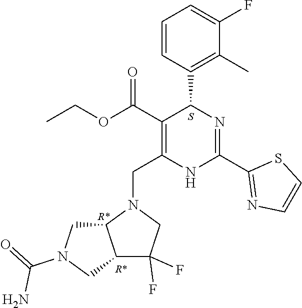 | 35 |
| 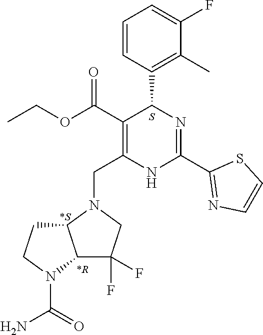 | 36 |
| 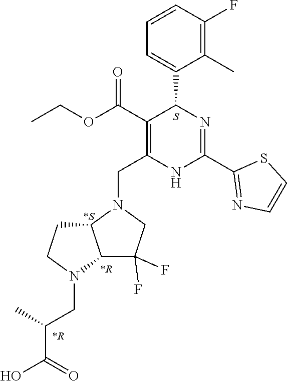 | 37E |
| 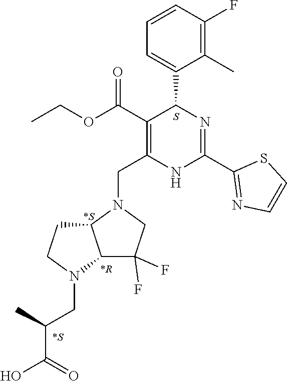 | 37F |
| 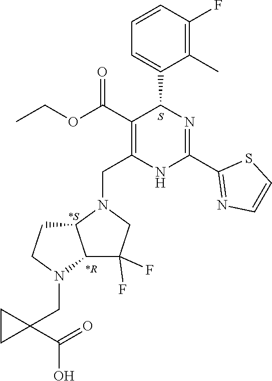 | 38A |
| 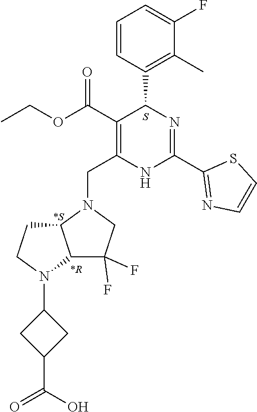 | 39A |
| 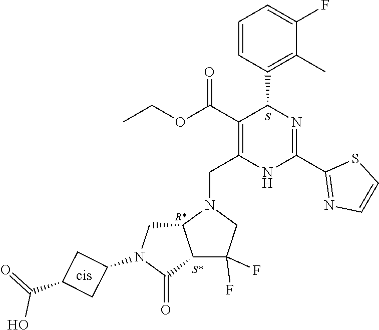 | 40 |
| 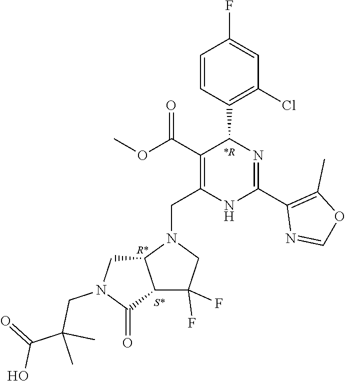 [SMILES: COC(=O)C1=C(CN2CC(F)(F)[C@@H]3C(=O)N(CC(C)(C)C(=O)O)C[C@@H]32)NC(c2ncoc2C)=N[C@H]1c1ccc(F)cc1Cl] | 41 |
| 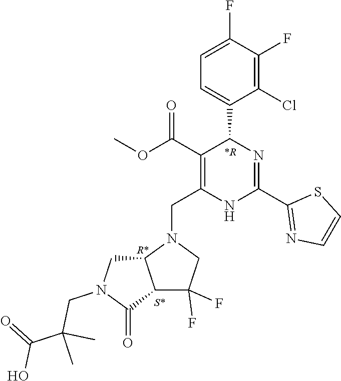 [SMILES: COC(=O)C1=C(CN2CC(F)(F)[C@@H]3C(=O)N(CC(C)(C)C(=O)O)C[C@@H]32)NC(c2nccs2)=N[C@H]1c1ccc(F)c(F)c1Cl] | 42 |
| 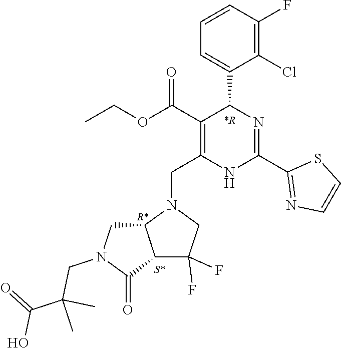 [SMILES: CCOC(=O)C1=C(CN2CC(F)(F)[C@@H]3C(=O)N(CC(C)(C)C(=O)O)C[C@@H]32)NC(c2nccs2)=N[C@H]1c1cccc(F)c1Cl] | 43 |
| 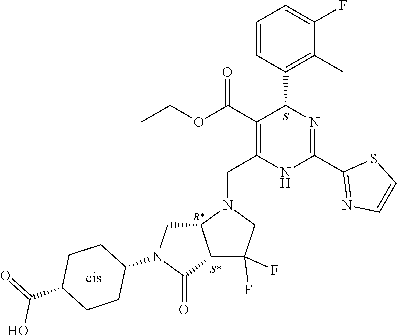 | 44A |
| 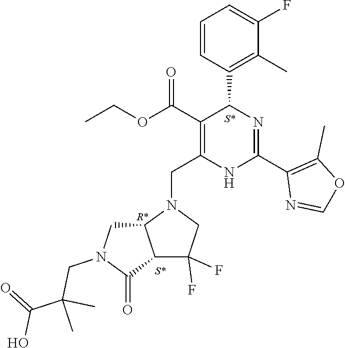 [SMILES: CCOC(=O)C1=C(CN2CC(F)(F)[C@@H]3C(=O)N(CC(C)(C)C(=O)O)C[C@@H]32)NC(c2ncoc2C)=N[C@H]1c1cccc(F)c1C] | 45 |
| 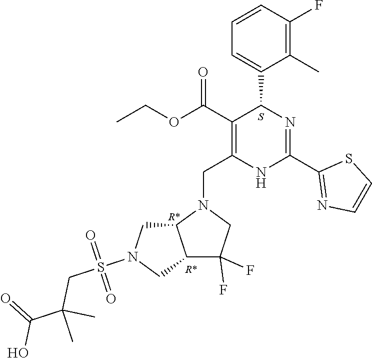 | 46 |
| 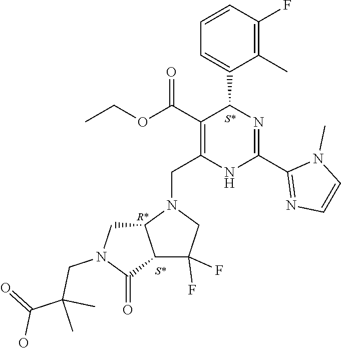 [SMILES: CCOC(=O)C1=C(CN2CC(F)(F)[C@@H]3C(=O)N(CC(C)(C)C(=O)O)C[C@@H]32)NC(c2nccn2C)=N[C@H]1c1cccc(F)c1C] | 47 |
| 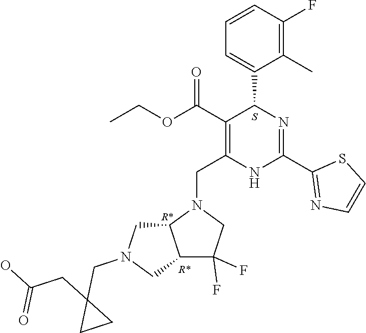 | 48 |
| 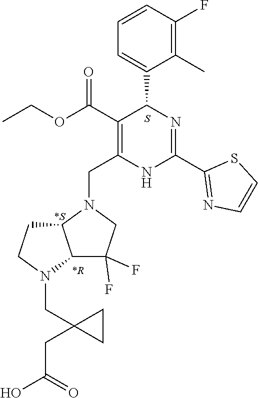 | 49 |
| 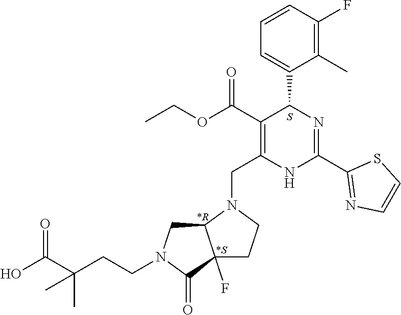 | 50A |
| 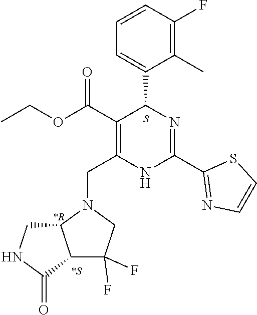 | 51 |
| 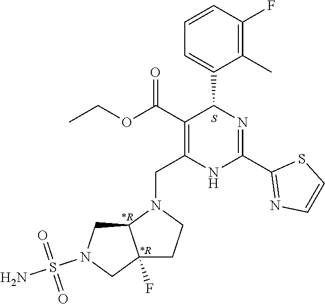 | 52A |
| 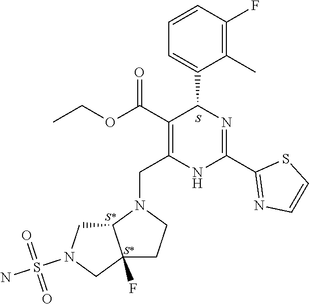 | 52B |
| 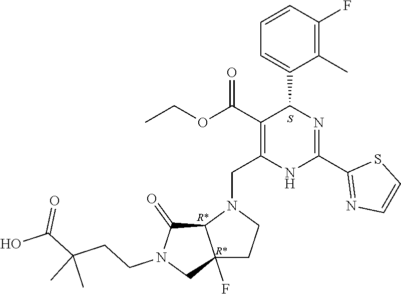 | 53A |
| 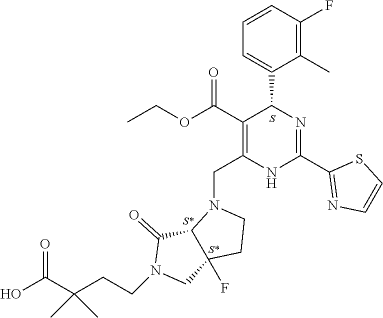 | 53B |
| 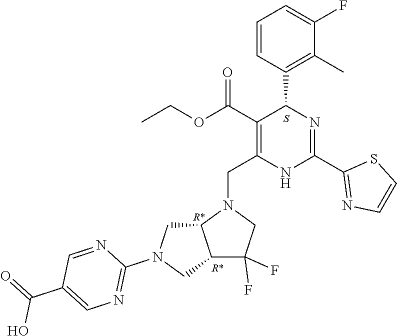 | 54 |
| 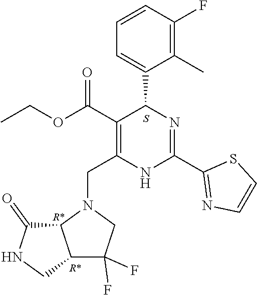 | 55 |
| 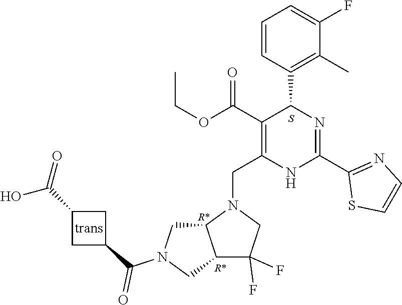 | 56A |
| 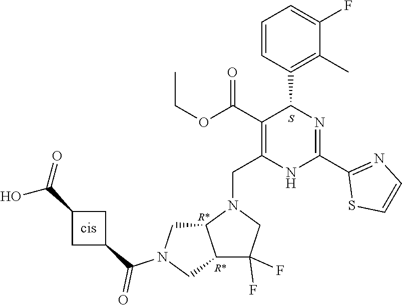 | 56A |
| 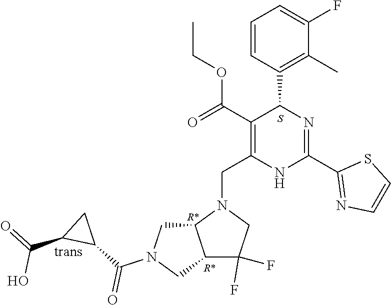 | 57A |
| 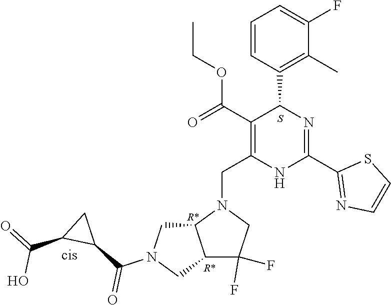 | 57B |
| 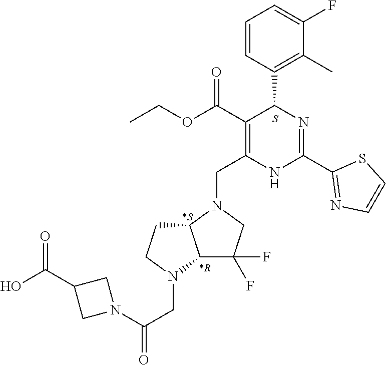 | 58 |
| 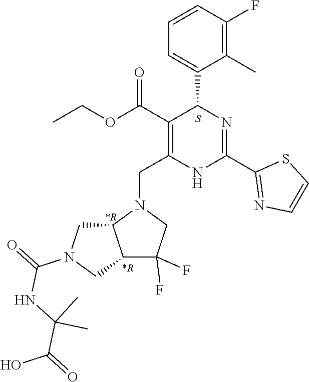 | 59 |
| 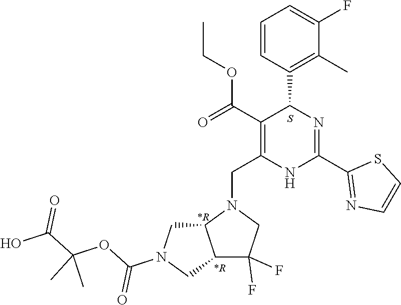 | 60 |
| 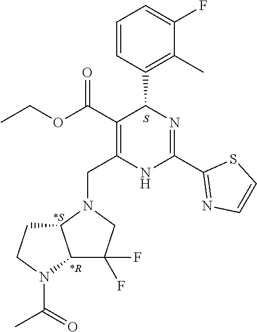 | 61 |
| 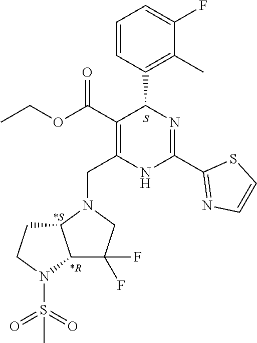 | 62 |
| 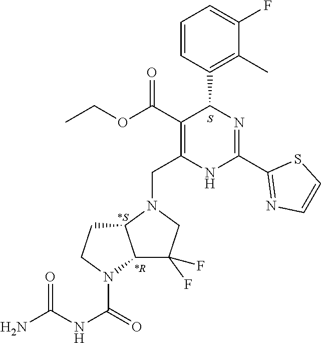 | 63 |
| 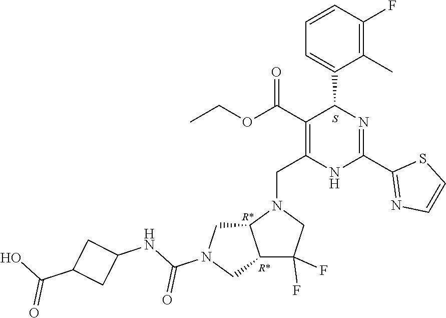 | 64 |
| 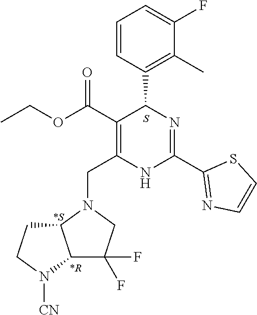 | 65 |
| 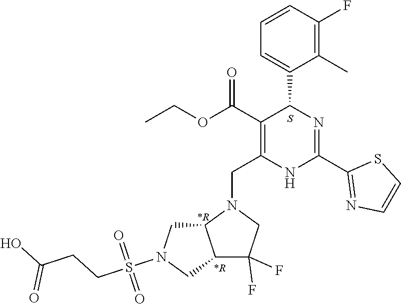 | 66 |
| 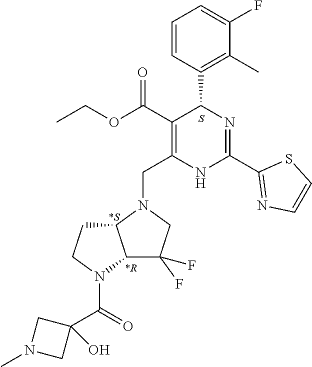 | 67 |
| 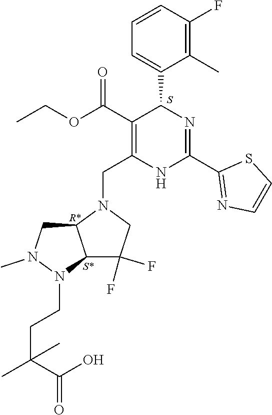 | 68A |
| 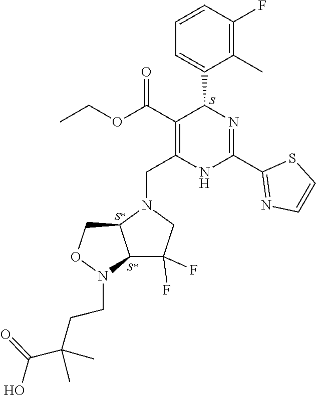 | 69A |
| 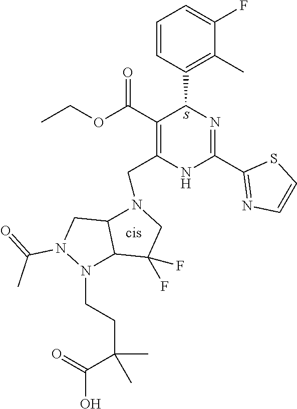 | 70 |
| 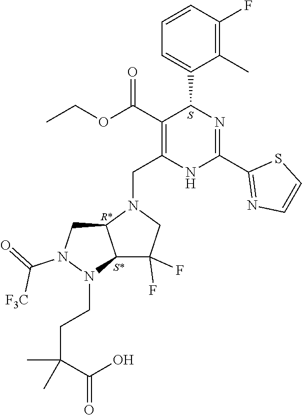 | 71 |
| 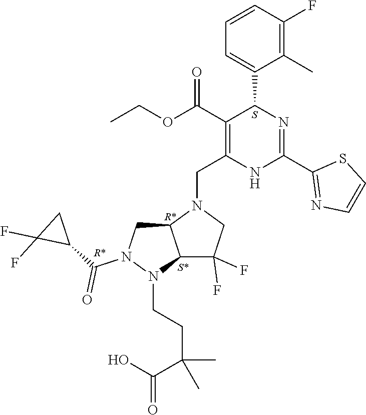 | 72A |
| 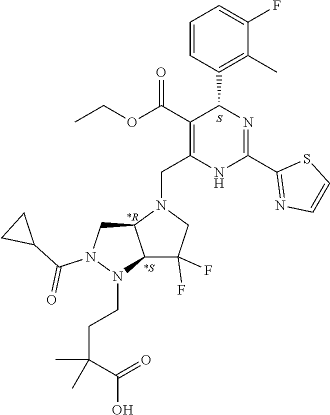 | 73 |
| 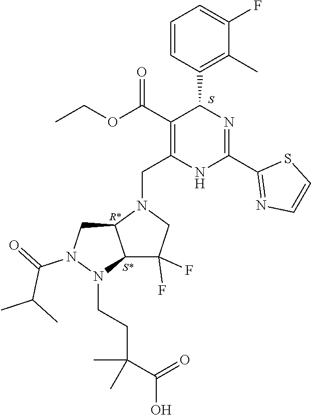 | 74 |
| 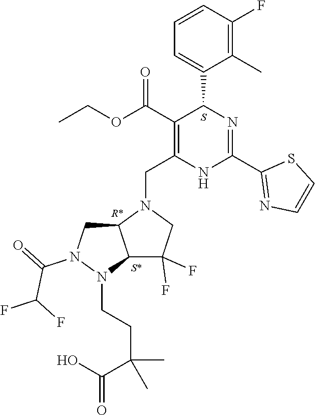 | 75 |
| 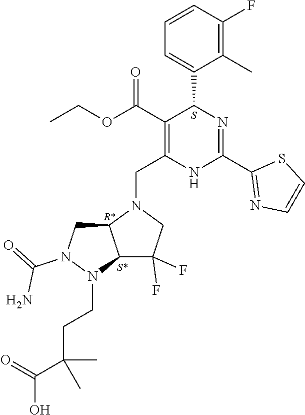 | 76A |
| 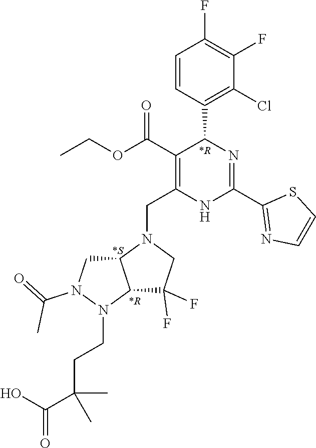 [SMILES: CCOC(=O)C1=C(CN2CC(F)(F)[C@H]3[C@@H]2CN(C(C)=O)N3CCC(C)(C)C(=O)O)NC(c2nccs2)=N[C@H]1c1ccc(F)c(F)c1Cl] | 77B |
| 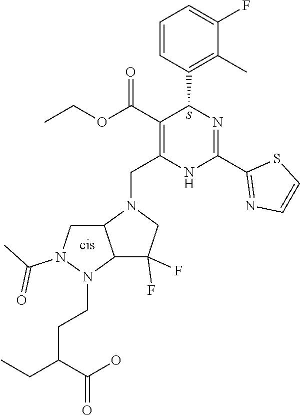 | 78 |
| 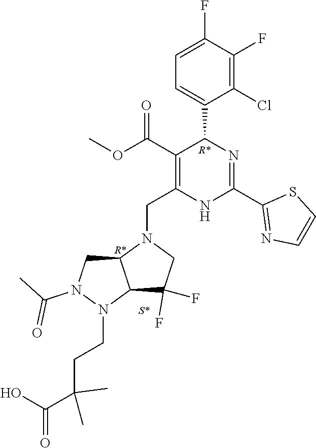 [SMILES: COC(=O)C1=C(CN2CC(F)(F)[C@@H]3[C@H]2CN(C(C)=O)N3CCC(C)(C)C(=O)O)NC(c2nccs2)=N[C@H]1c1ccc(F)c(F)c1Cl] | 79A |
| 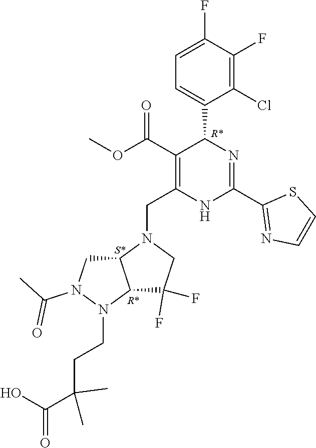 [SMILES: COC(=O)C1=C(CN2CC(F)(F)[C@H]3[C@@H]2CN(C(C)=O)N3CCC(C)(C)C(=O)O)NC(c2nccs2)=N[C@H]1c1ccc(F)c(F)c1Cl] | 79B |
| 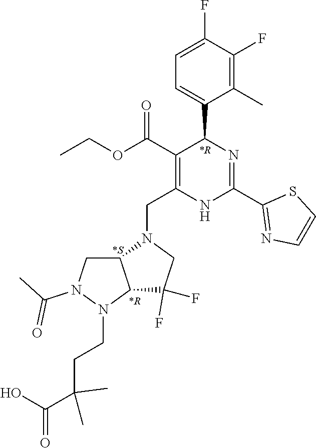 [SMILES: CCOC(=O)C1=C(CN2CC(F)(F)[C@H]3[C@@H]2CN(C(C)=O)N3CCC(C)(C)C(=O)O)NC(c2nccs2)=N[C@@H]1c1ccc(F)c(F)c1C] | 80B |
| 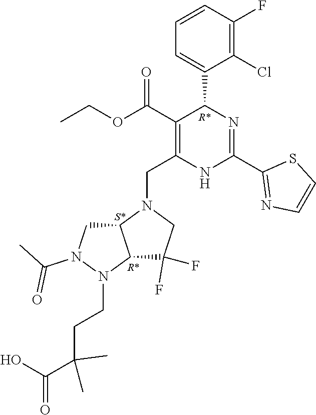 [SMILES: CCOC(=O)C1=C(CN2CC(F)(F)[C@H]3[C@@H]2CN(C(C)=O)N3CCC(C)(C)C(=O)O)NC(c2nccs2)=N[C@H]1c1cccc(F)c1Cl] | 81B |
| 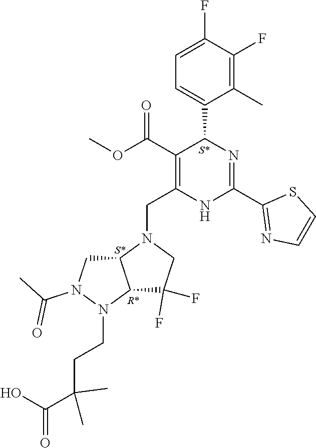 [SMILES: COC(=O)C1=C(CN2CC(F)(F)[C@H]3[C@@H]2CN(C(C)=O)N3CCC(C)(C)C(=O)O)NC(c2nccs2)=N[C@H]1c1ccc(F)c(F)c1C] | 82B |
| 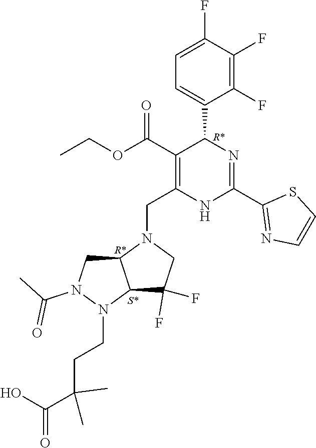 [SMILES: CCOC(=O)C1=C(CN2CC(F)(F)[C@@H]3[C@H]2CN(C(C)=O)N3CCC(C)(C)C(=O)O)NC(c2nccs2)=N[C@H]1c1ccc(F)c(F)c1F] | 83A |
| 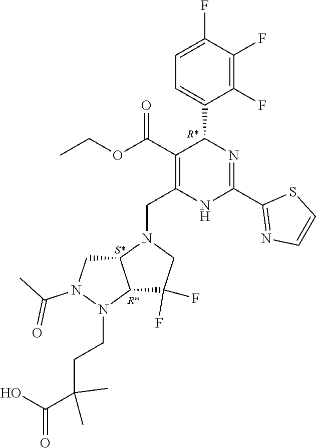 [SMILES: CCOC(=O)C1=C(CN2CC(F)(F)[C@H]3[C@@H]2CN(C(C)=O)N3CCC(C)(C)C(=O)O)NC(c2nccs2)=N[C@H]1c1ccc(F)c(F)c1F] | 83B |
| 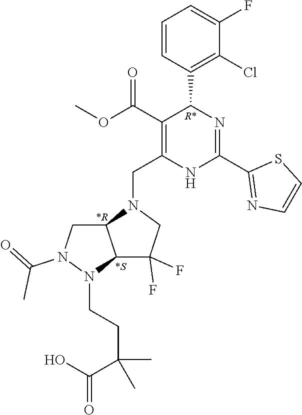 [SMILES: COC(=O)C1=C(CN2CC(F)(F)[C@@H]3[C@H]2CN(C(C)=O)N3CCC(C)(C)C(=O)O)NC(c2nccs2)=N[C@H]1c1cccc(F)c1Cl] | 84A |
| 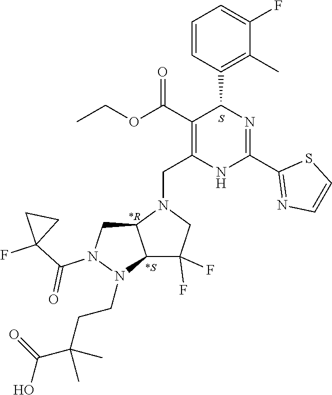 | 85 |
| 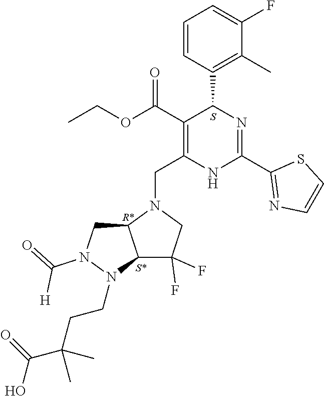 | 86 |
| 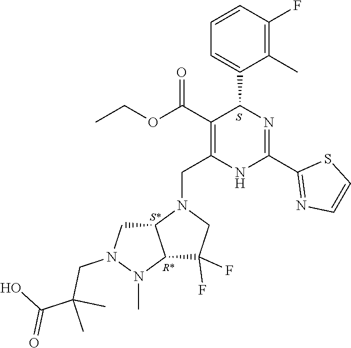 | 87 |
| 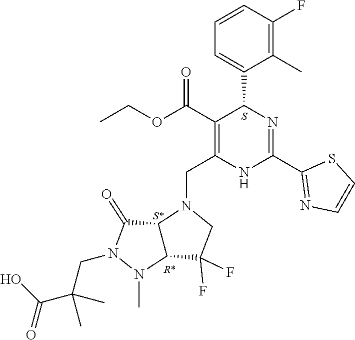 | 88 |
| 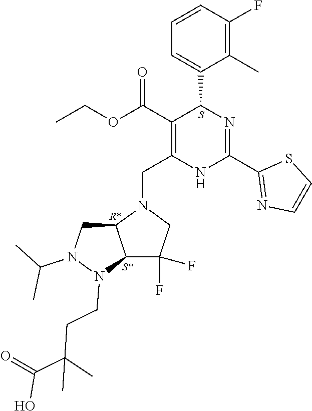 | 89 |
| 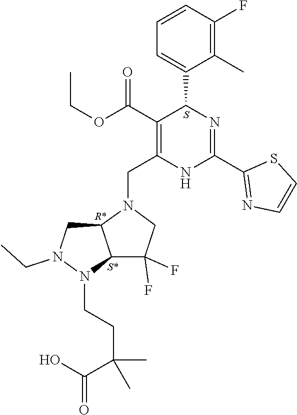 | 90 |
| 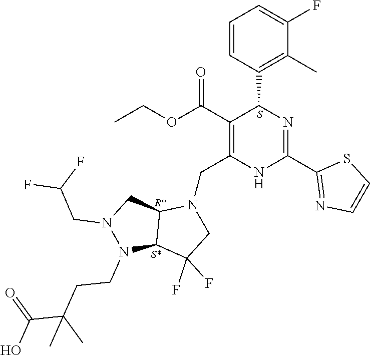 | 91 |
| 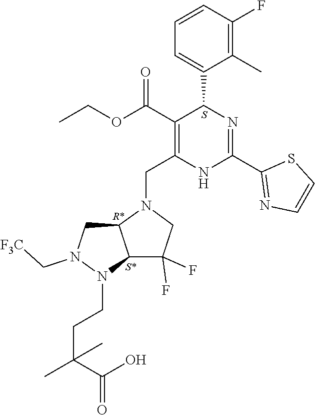 | 92 |
| 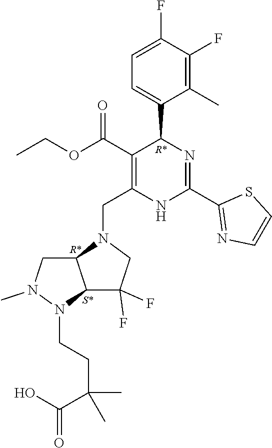 [SMILES: CCOC(=O)C1=C(CN2CC(F)(F)[C@@H]3[C@H]2CN(C)N3CCC(C)(C)C(=O)O)NC(c2nccs2)=N[C@@H]1c1ccc(F)c(F)c1C] | 93 |
| 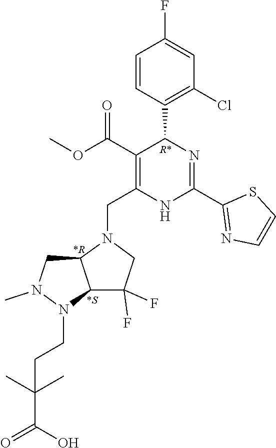 [SMILES: COC(=O)C1=C(CN2CC(F)(F)[C@@H]3[C@H]2CN(C)N3CCC(C)(C)C(=O)O)NC(c2nccs2)=N[C@H]1c1ccc(F)cc1Cl] | 94 |
| 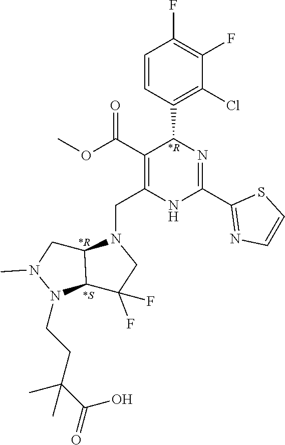 [SMILES: COC(=O)C1=C(CN2CC(F)(F)[C@@H]3[C@H]2CN(C)N3CCC(C)(C)C(=O)O)NC(c2nccs2)=N[C@H]1c1ccc(F)c(F)c1Cl] | 95 |
| 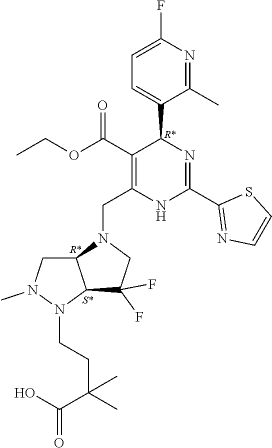 [SMILES: CCOC(=O)C1=C(CN2CC(F)(F)[C@@H]3[C@H]2CN(C)N3CCC(C)(C)C(=O)O)NC(c2nccs2)=N[C@@H]1c1ccc(F)nc1C] | 96 |
| 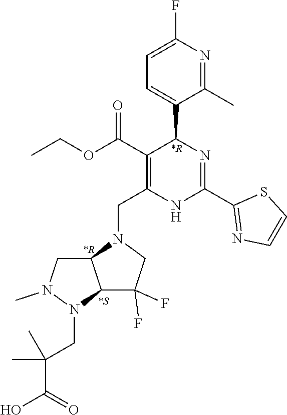 [SMILES: CCOC(=O)C1=C(CN2CC(F)(F)[C@@H]3[C@H]2CN(C)N3CC(C)(C)C(=O)O)NC(c2nccs2)=N[C@@H]1c1ccc(F)nc1C] | 97 |
| 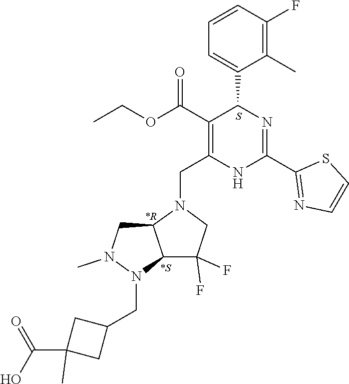 | 98 |
| 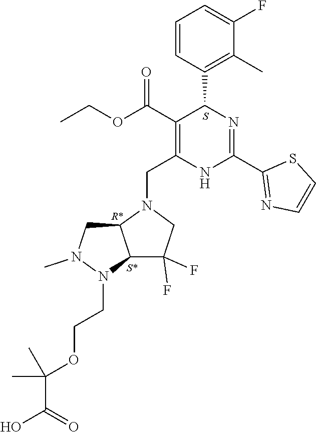 | 99 |
| 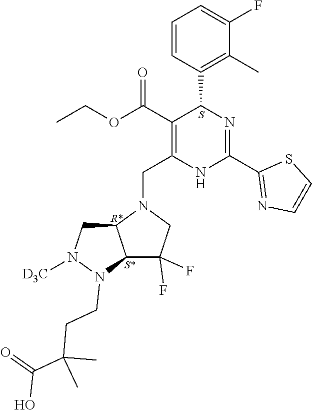 | 100 |
| 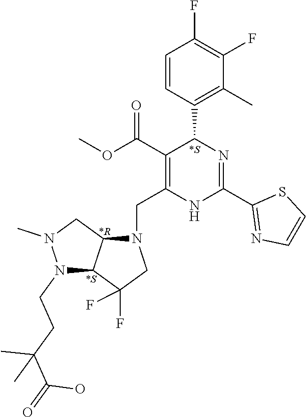 | 101 |
| 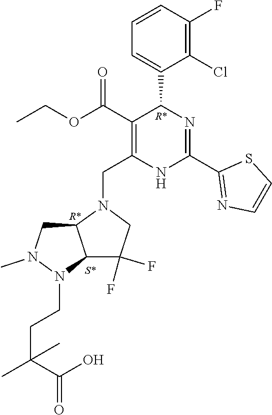 [SMILES: CCOC(=O)C1=C(CN2CC(F)(F)[C@@H]3[C@H]2CN(C)N3CCC(C)(C)C(=O)O)NC(c2nccs2)=N[C@H]1c1cccc(F)c1Cl] | 102 |
| 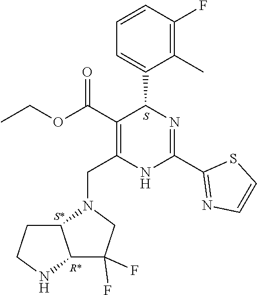 | 103 |
| 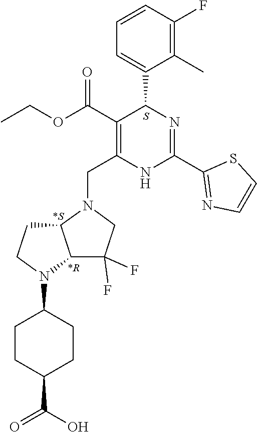 | 104A |
| 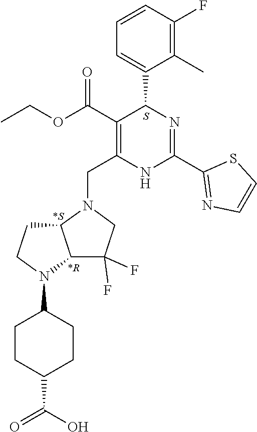 | 104B |
| 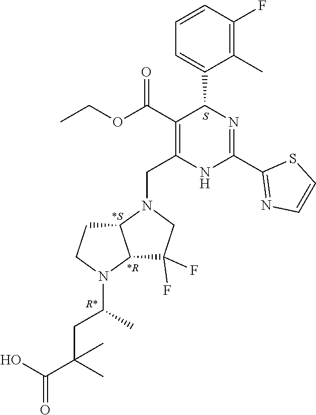 | 105A |
| 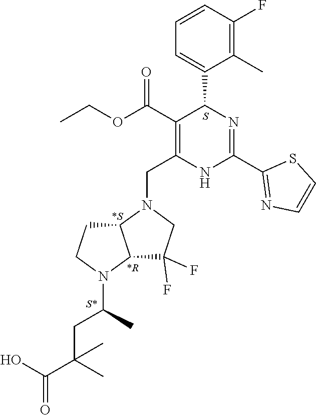 | 105B |
| 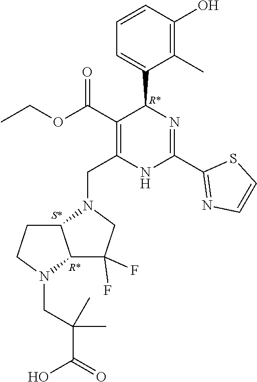 [SMILES: CCOC(=O)C1=C(CN2CC(F)(F)[C@H]3[C@@H]2CCN3CC(C)(C)C(=O)O)NC(c2nccs2)=N[C@@H]1c1cccc(O)c1C] | 106 |
| 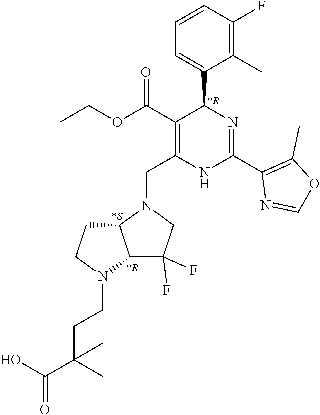 [SMILES: CCOC(=O)C1=C(CN2CC(F)(F)[C@H]3[C@@H]2CCN3CCC(C)(C)C(=O)O)NC(c2ncoc2C)=N[C@@H]1c1cccc(F)c1C] | 107 |
| 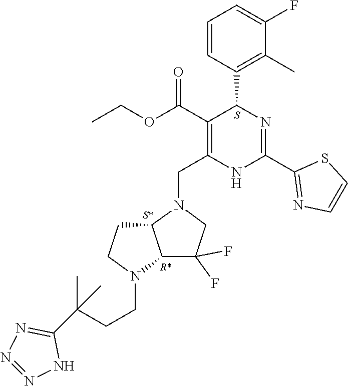 | 108 |
| 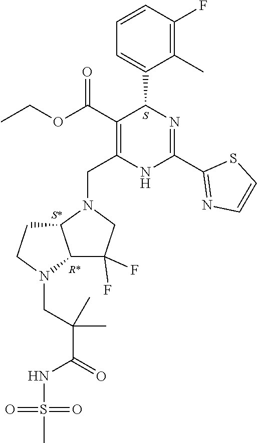 | 109 |
| 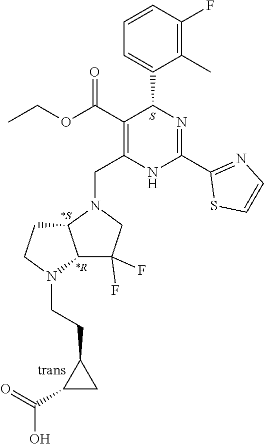 | 110 |
| 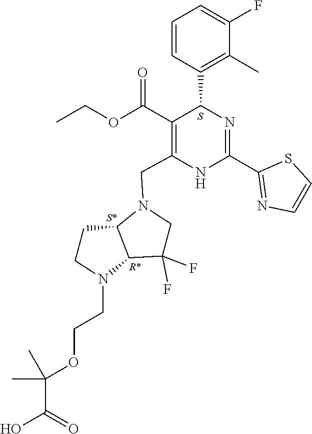 | 111 |
| 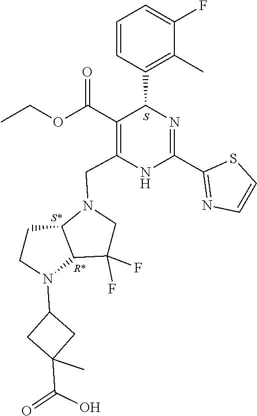 | 112 |
| 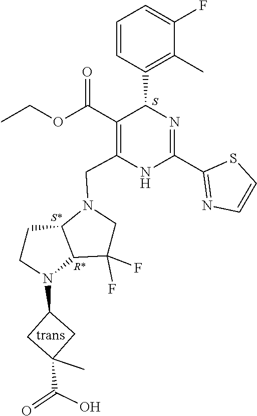 | 112A |
| 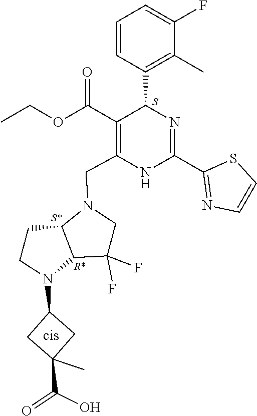 | 112B |
| 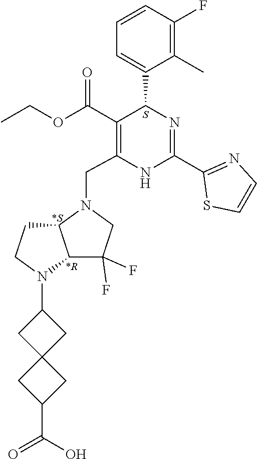 | 113 |
| 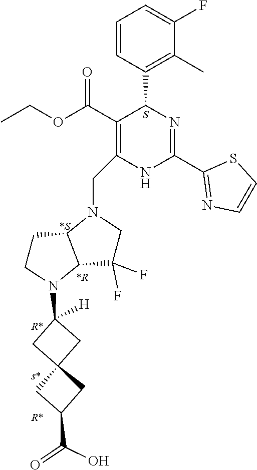 | 113A |
| 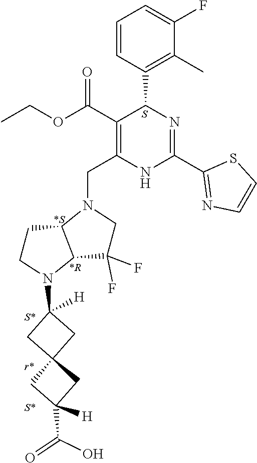 | 113B |
| 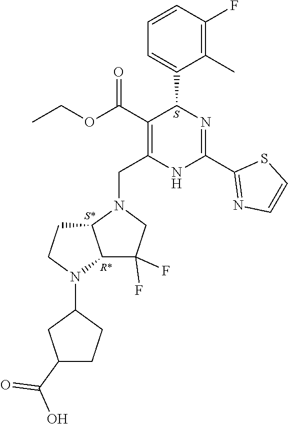 | 115 |
| 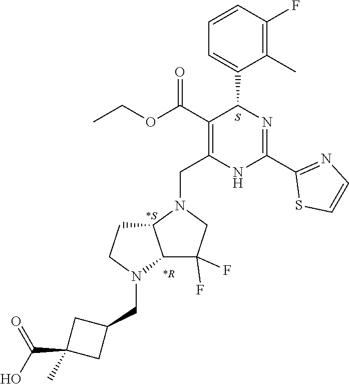 | 116A |
| 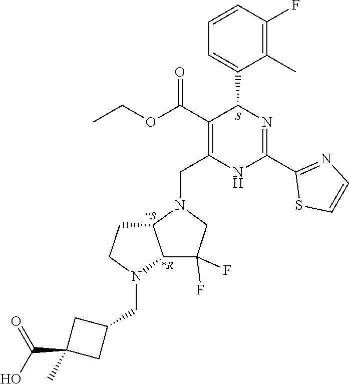 | 116B |
| 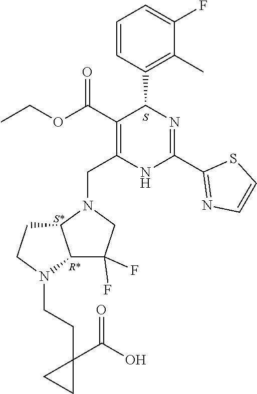 | 117 |
| 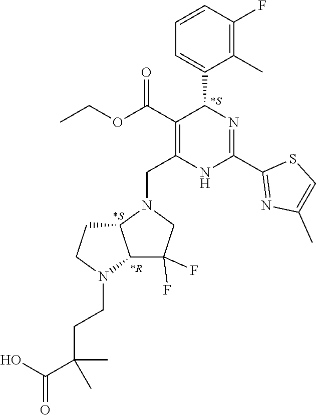 [SMILES: CCOC(=O)C1=C(CN2CC(F)(F)[C@H]3[C@@H]2CCN3CCC(C)(C)C(=O)O)NC(c2nc(C)cs2)=N[C@H]1c1cccc(F)c1C] | 118 |
| 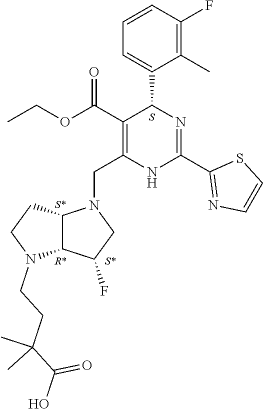 | 119 |
| 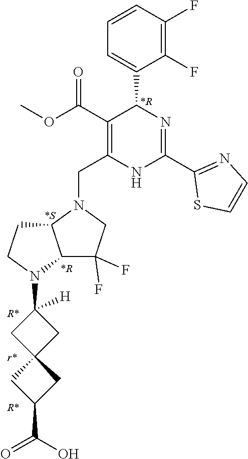 | 120A |
| 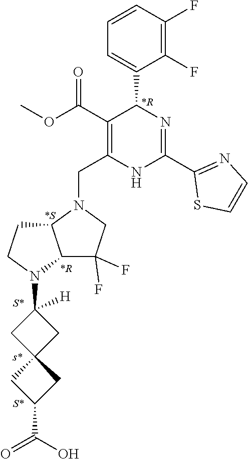 | 120B |
| 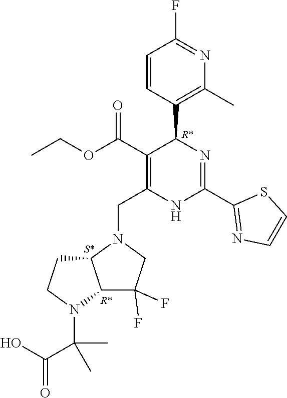 [SMILES: CCOC(=O)C1=C(CN2CC(F)(F)[C@H]3[C@@H]2CCN3C(C)(C)C(=O)O)NC(c2nccs2)=N[C@@H]1c1ccc(F)nc1C] | 121 |
| 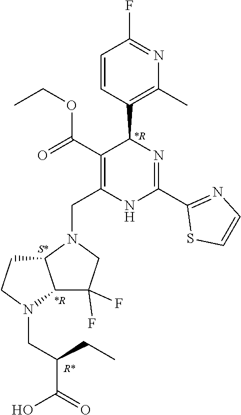 [SMILES: CCOC(=O)C1=C(CN2CC(F)(F)[C@H]3[C@@H]2CCN3C[C@@H](CC)C(=O)O)NC(c2nccs2)=N[C@@H]1c1ccc(F)nc1C] | 122A |
| 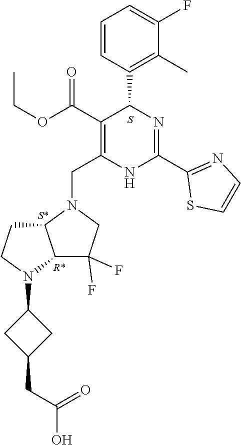 | 123A |
| 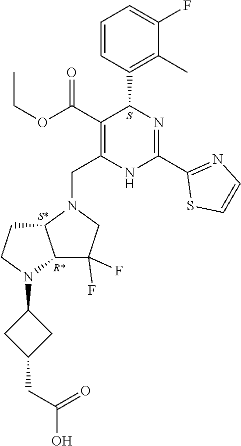 | 123B |
| 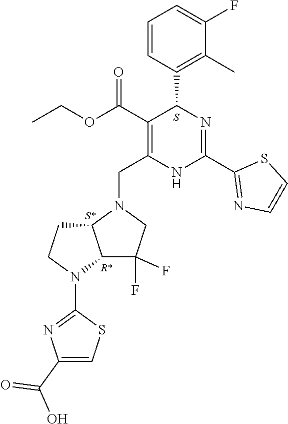 | 124 |
| 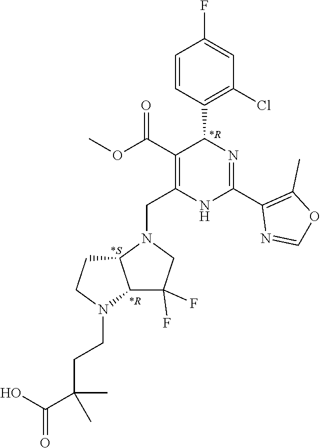 [SMILES: COC(=O)C1=C(CN2CC(F)(F)[C@H]3[C@@H]2CCN3CCC(C)(C)C(=O)O)NC(c2ncoc2C)=N[C@H]1c1ccc(F)cc1Cl] | 125 |
| 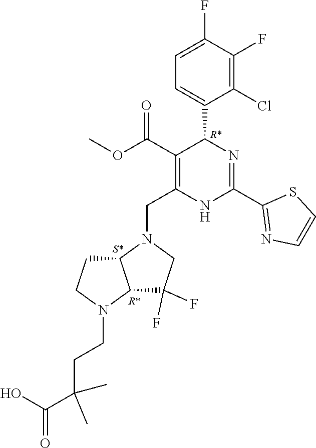 [SMILES: COC(=O)C1=C(CN2CC(F)(F)[C@H]3[C@@H]2CCN3CCC(C)(C)C(=O)O)NC(c2nccs2)=N[C@H]1c1ccc(F)c(F)c1Cl] | 126 |
| 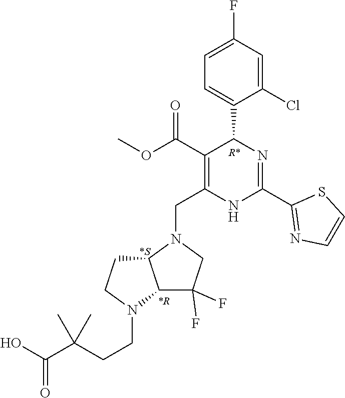 [SMILES: COC(=O)C1=C(CN2CC(F)(F)[C@H]3[C@@H]2CCN3CCC(C)(C)C(=O)O)NC(c2nccs2)=N[C@H]1c1ccc(F)cc1Cl] | 127 |
| 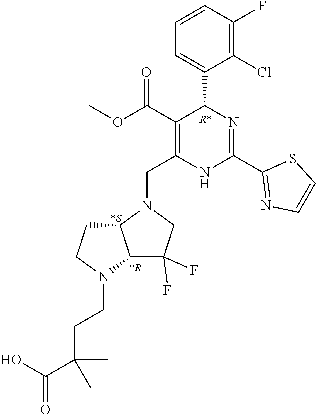 [SMILES: COC(=O)C1=C(CN2CC(F)(F)[C@H]3[C@@H]2CCN3CCC(C)(C)C(=O)O)NC(c2nccs2)=N[C@H]1c1cccc(F)c1Cl] | 128 |
| 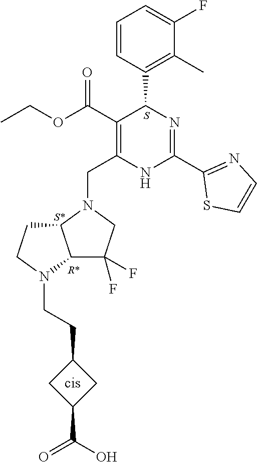 | 129A |
| 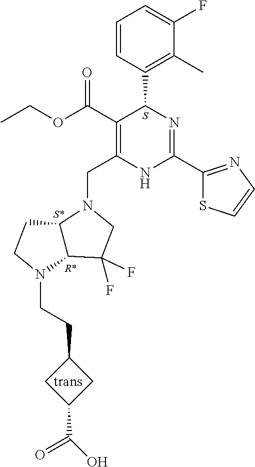 | 129B |
| 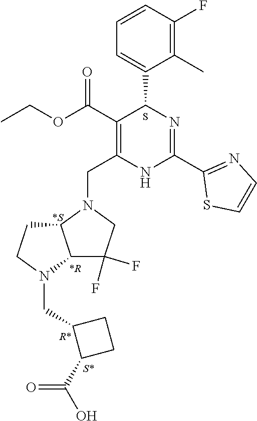 | 130A |
| 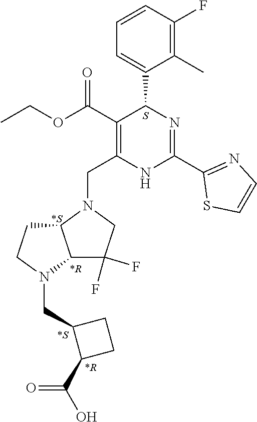 | 130B |
| 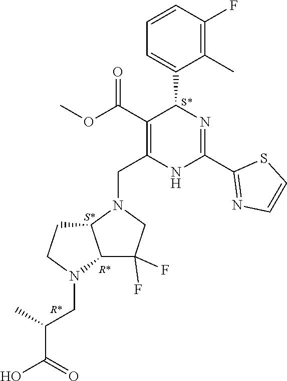 [SMILES: COC(=O)C1=C(CN2CC(F)(F)[C@H]3[C@@H]2CCN3C[C@@H](C)C(=O)O)NC(c2nccs2)=N[C@H]1c1cccc(F)c1C] | 131A |
| 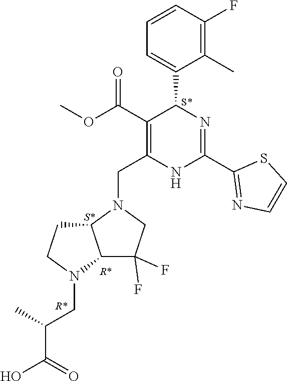 [SMILES: COC(=O)C1=C(CN2CC(F)(F)[C@H]3[C@@H]2CCN3C[C@@H](C)C(=O)O)NC(c2nccs2)=N[C@H]1c1cccc(F)c1C] | 131B |
| 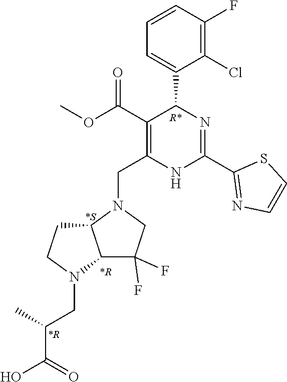 [SMILES: COC(=O)C1=C(CN2CC(F)(F)[C@H]3[C@@H]2CCN3C[C@@H](C)C(=O)O)NC(c2nccs2)=N[C@H]1c1cccc(F)c1Cl] | 132A |
| 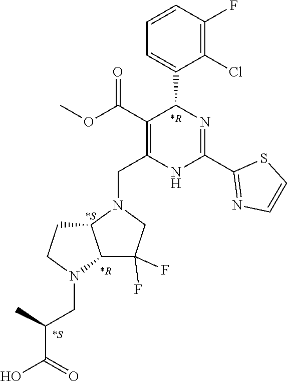 [SMILES: COC(=O)C1=C(CN2CC(F)(F)[C@H]3[C@@H]2CCN3C[C@H](C)C(=O)O)NC(c2nccs2)=N[C@H]1c1cccc(F)c1Cl] | 132B |
| 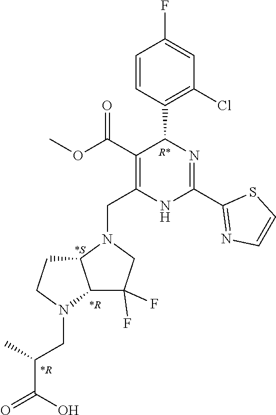 [SMILES: COC(=O)C1=C(CN2CC(F)(F)[C@H]3[C@@H]2CCN3C[C@@H](C)C(=O)O)NC(c2nccs2)=N[C@H]1c1ccc(F)cc1Cl] | 133A |
| 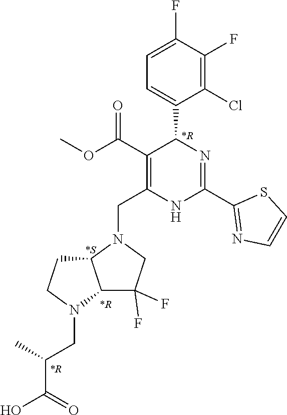 [SMILES: COC(=O)C1=C(CN2CC(F)(F)[C@H]3[C@@H]2CCN3C[C@@H](C)C(=O)O)NC(c2nccs2)=N[C@H]1c1ccc(F)c(F)c1Cl] | 134A |
| 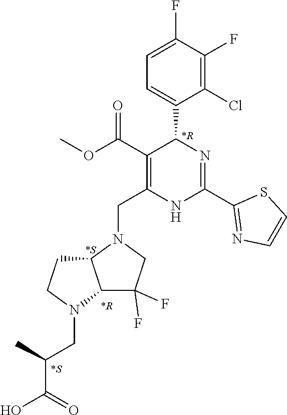 [SMILES: COC(=O)C1=C(CN2CC(F)(F)[C@H]3[C@@H]2CCN3C[C@H](C)C(=O)O)NC(c2nccs2)=N[C@H]1c1ccc(F)c(F)c1Cl] | 134B |
| 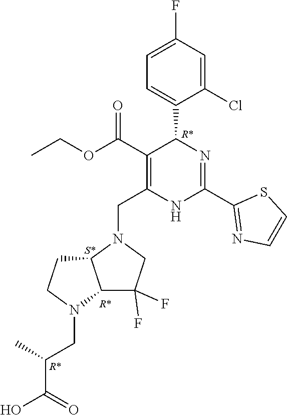 [SMILES: CCOC(=O)C1=C(CN2CC(F)(F)[C@H]3[C@@H]2CCN3C[C@@H](C)C(=O)O)NC(c2nccs2)=N[C@H]1c1ccc(F)cc1Cl] | 135A |
| 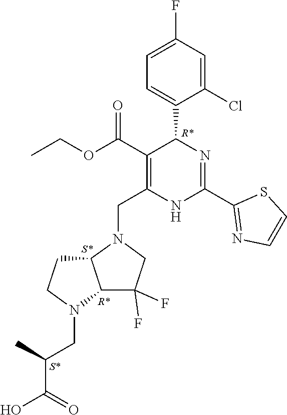 [SMILES: CCOC(=O)C1=C(CN2CC(F)(F)[C@H]3[C@@H]2CCN3C[C@H](C)C(=O)O)NC(c2nccs2)=N[C@H]1c1ccc(F)cc1Cl] | 135B |
| 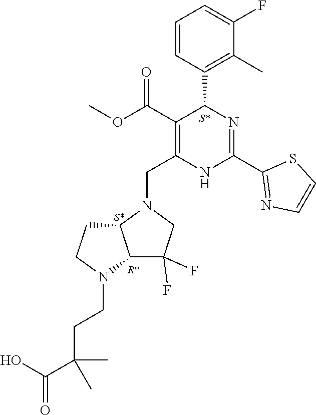 [SMILES: COC(=O)C1=C(CN2CC(F)(F)[C@H]3[C@@H]2CCN3CCC(C)(C)C(=O)O)NC(c2nccs2)=N[C@H]1c1cccc(F)c1C] | 136 |
| 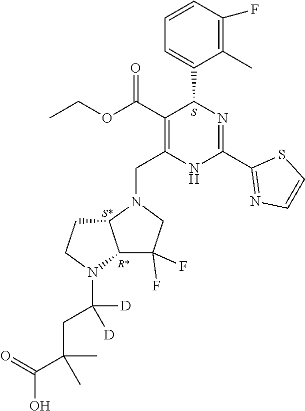 | 137 |
| 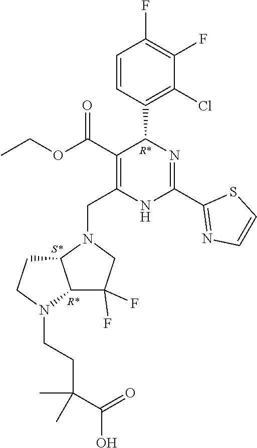 [SMILES: CCOC(=O)C1=C(CN2CC(F)(F)[C@H]3[C@@H]2CCN3CCC(C)(C)C(=O)O)NC(c2nccs2)=N[C@H]1c1ccc(F)c(F)c1Cl] | 138 |
| 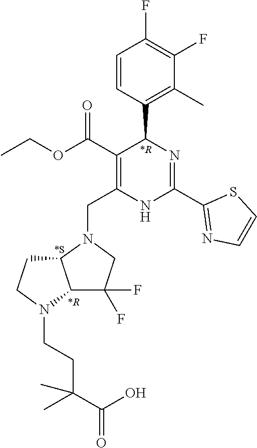 [SMILES: CCOC(=O)C1=C(CN2CC(F)(F)[C@H]3[C@@H]2CCN3CCC(C)(C)C(=O)O)NC(c2nccs2)=N[C@@H]1c1ccc(F)c(F)c1C] | 139 |
| 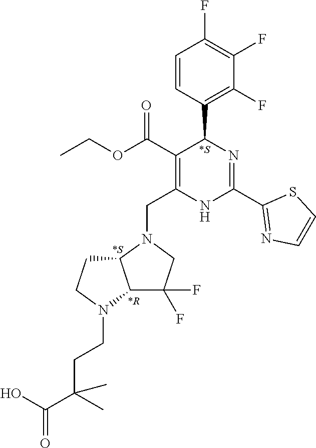 [SMILES: CCOC(=O)C1=C(CN2CC(F)(F)[C@H]3[C@@H]2CCN3CCC(C)(C)C(=O)O)NC(c2nccs2)=N[C@@H]1c1ccc(F)c(F)c1F] | 140 |
| 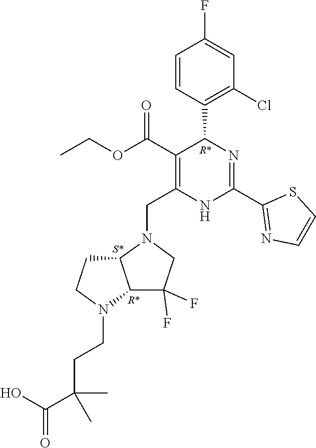 [SMILES: CCOC(=O)C1=C(CN2CC(F)(F)[C@H]3[C@@H]2CCN3CCC(C)(C)C(=O)O)NC(c2nccs2)=N[C@H]1c1ccc(F)cc1Cl] | 141 |
| 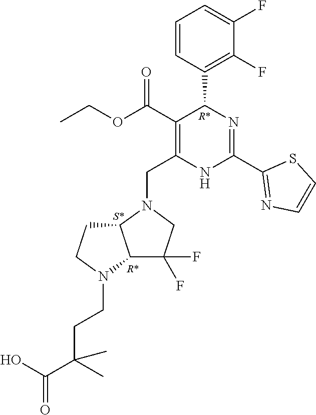 [SMILES: CCOC(=O)C1=C(CN2CC(F)(F)[C@H]3[C@@H]2CCN3CCC(C)(C)C(=O)O)NC(c2nccs2)=N[C@H]1c1cccc(F)c1F] | 142 |
| 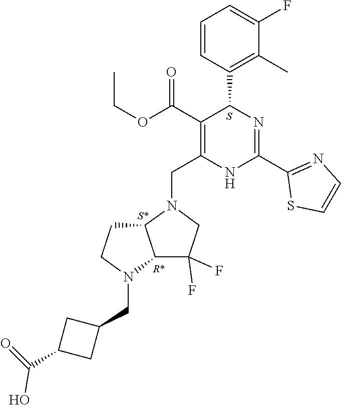 | 143A |
| 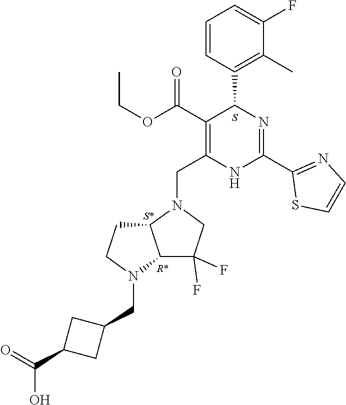 | 143B |
| 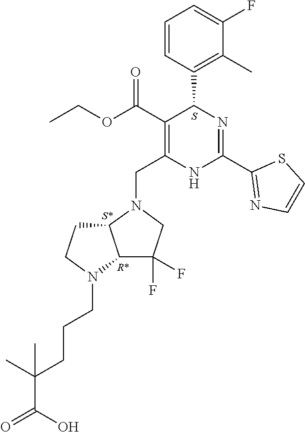 | 144 |
| 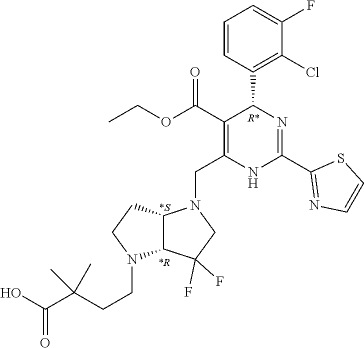 [SMILES: CCOC(=O)C1=C(CN2CC(F)(F)[C@H]3[C@@H]2CCN3CCC(C)(C)C(=O)O)NC(c2nccs2)=N[C@H]1c1cccc(F)c1Cl] | 145 |
| 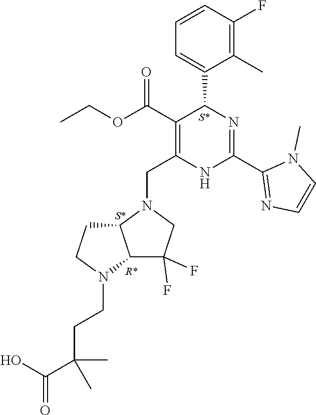 [SMILES: CCOC(=O)C1=C(CN2CC(F)(F)[C@H]3[C@@H]2CCN3CCC(C)(C)C(=O)O)NC(c2nccn2C)=N[C@H]1c1cccc(F)c1C] | 146 |
| 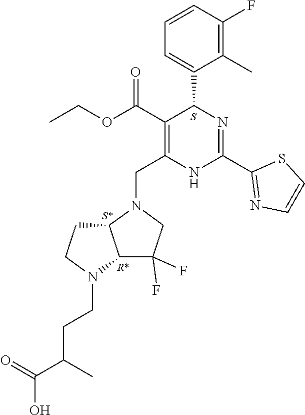 | 147 |
| 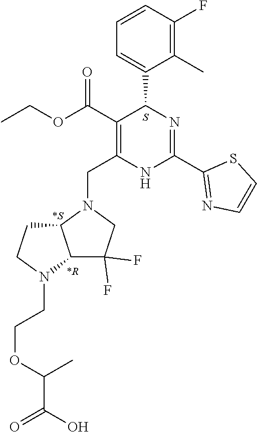 | 148 |
| 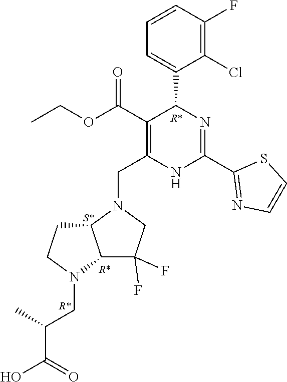 [SMILES: CCOC(=O)C1=C(CN2CC(F)(F)[C@H]3[C@@H]2CCN3C[C@@H](C)C(=O)O)NC(c2nccs2)=N[C@H]1c1cccc(F)c1Cl] | 149A |
| 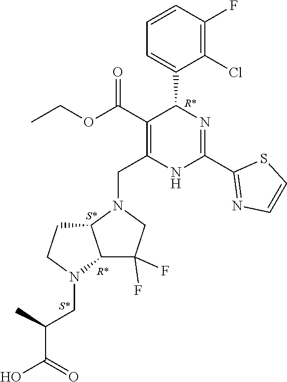 [SMILES: CCOC(=O)C1=C(CN2CC(F)(F)[C@H]3[C@@H]2CCN3C[C@H](C)C(=O)O)NC(c2nccs2)=N[C@H]1c1cccc(F)c1Cl] | 149B |
| 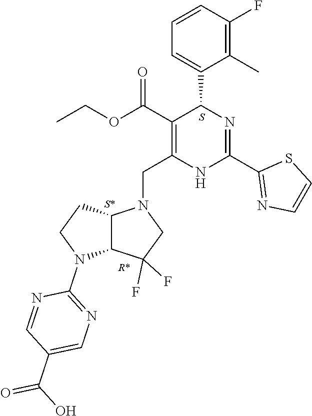 | 150 |
| 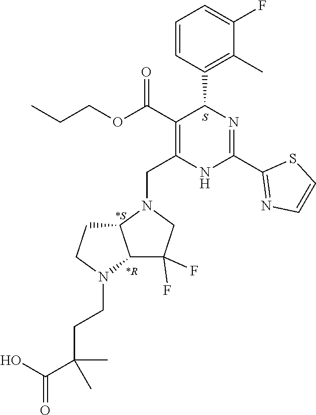 | 151 |
| 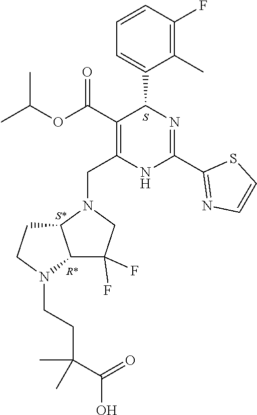 | 152 |
| 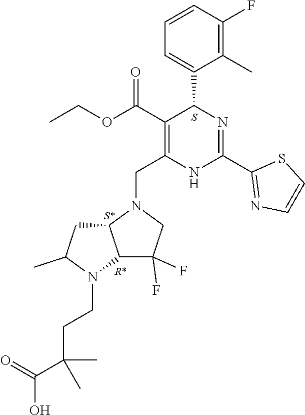 | 153 |
| 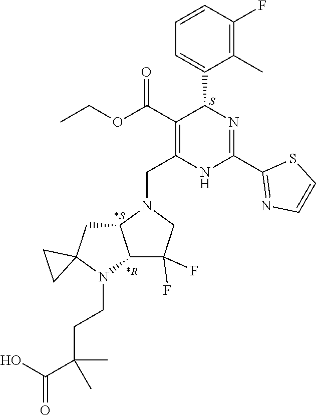 | 154 |
| 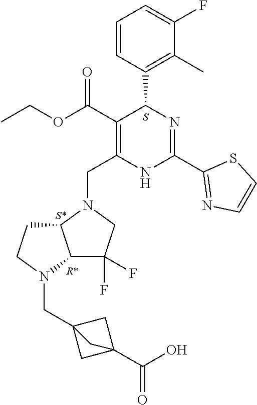 | 155 |
| 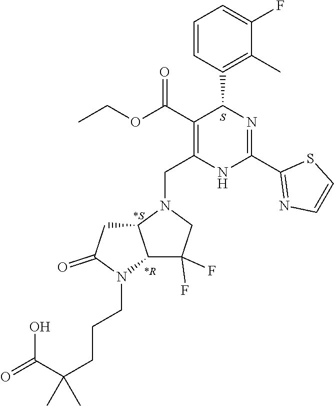 | 156 |
| 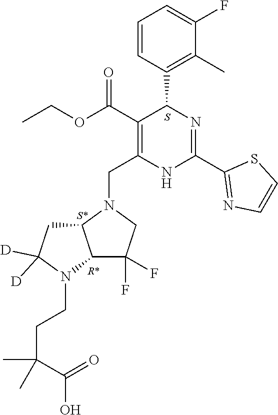 | 157 |
| 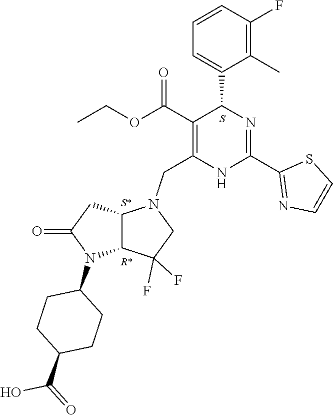 | 158A |
| 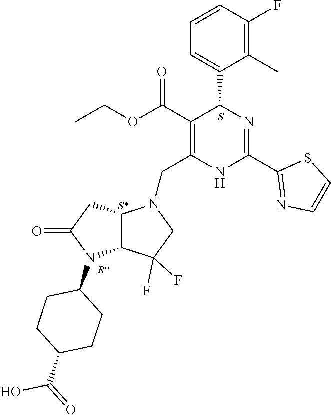 | 158B |
| 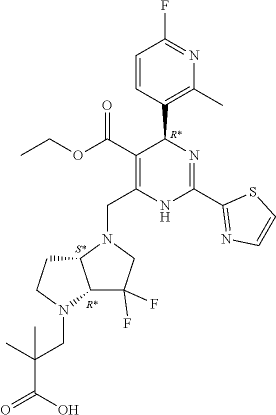 [SMILES: CCOC(=O)C1=C(CN2CC(F)(F)[C@H]3[C@@H]2CCN3CC(C)(C)C(=O)O)NC(c2nccs2)=N[C@@H]1c1ccc(F)nc1C] | 159 |
| 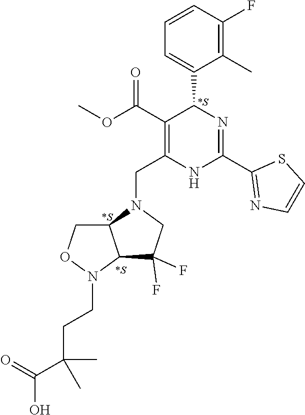 | 160 |
| 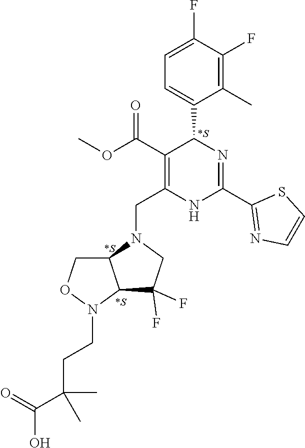 [SMILES: COC(=O)C1=C(CN2CC(F)(F)[C@@H]3[C@H]2CON3CCC(C)(C)C(=O)O)NC(c2nccs2)=N[C@H]1c1ccc(F)c(F)c1C] | 161 |
| 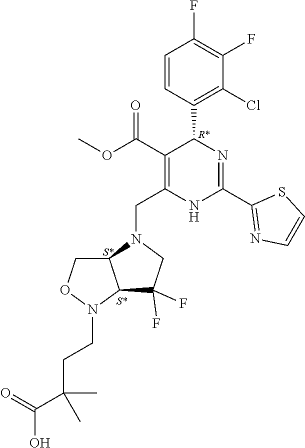 [SMILES: COC(=O)C1=C(CN2CC(F)(F)[C@@H]3[C@H]2CON3CCC(C)(C)C(=O)O)NC(c2nccs2)=N[C@H]1c1ccc(F)c(F)c1Cl] | 162 |
| 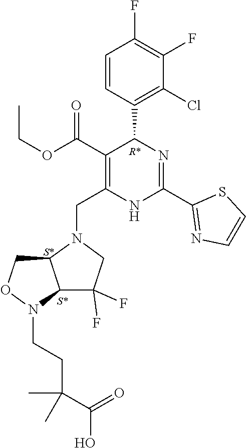 [SMILES: CCOC(=O)C1=C(CN2CC(F)(F)[C@@H]3[C@H]2CON3CCC(C)(C)C(=O)O)NC(c2nccs2)=N[C@H]1c1ccc(F)c(F)c1Cl] | 163 |
| 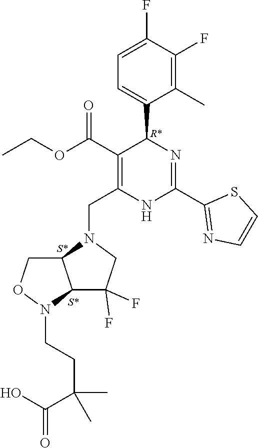 [SMILES: CCOC(=O)C1=C(CN2CC(F)(F)[C@@H]3[C@H]2CON3CCC(C)(C)C(=O)O)NC(c2nccs2)=N[C@@H]1c1ccc(F)c(F)c1C] | 164 |
| 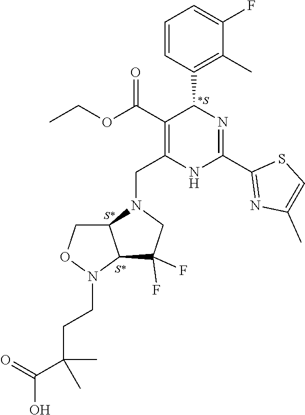 [SMILES: CCOC(=O)C1=C(CN2CC(F)(F)[C@@H]3[C@H]2CON3CCC(C)(C)C(=O)O)NC(c2nc(C)cs2)=N[C@H]1c1cccc(F)c1C] | 165 |
| 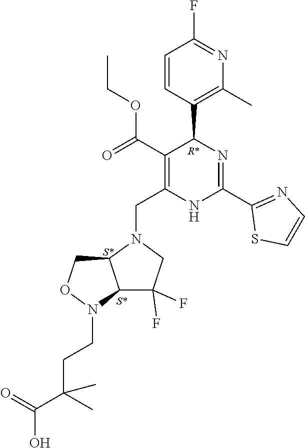 [SMILES: CCOC(=O)C1=C(CN2CC(F)(F)[C@@H]3[C@H]2CON3CCC(C)(C)C(=O)O)NC(c2nccs2)=N[C@@H]1c1ccc(F)nc1C] | 166 |
| 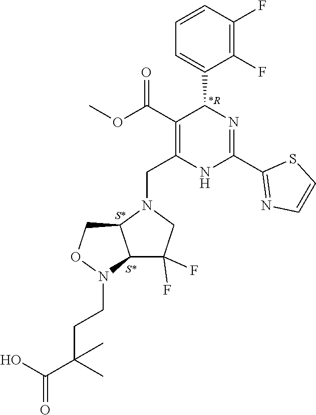 [SMILES: COC(=O)C1=C(CN2CC(F)(F)[C@@H]3[C@H]2CON3CCC(C)(C)C(=O)O)NC(c2nccs2)=N[C@H]1c1cccc(F)c1F] | 167 |
| 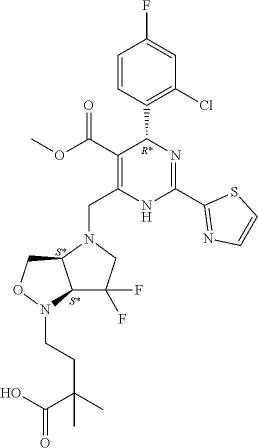 [SMILES: COC(=O)C1=C(CN2CC(F)(F)[C@@H]3[C@H]2CON3CCC(C)(C)C(=O)O)NC(c2nccs2)=N[C@H]1c1ccc(F)cc1Cl] | 168 |
| 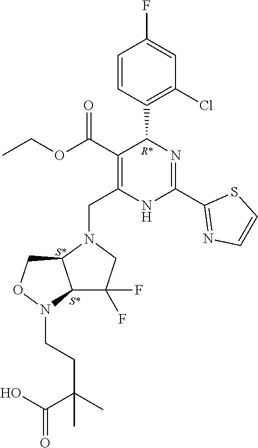 [SMILES: CCOC(=O)C1=C(CN2CC(F)(F)[C@@H]3[C@H]2CON3CCC(C)(C)C(=O)O)NC(c2nccs2)=N[C@H]1c1ccc(F)cc1Cl] | 169 |
| 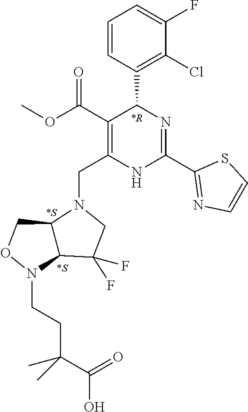 [SMILES: COC(=O)C1=C(CN2CC(F)(F)[C@@H]3[C@H]2CON3CCC(C)(C)C(=O)O)NC(c2nccs2)=N[C@H]1c1cccc(F)c1Cl] | 170 |
| 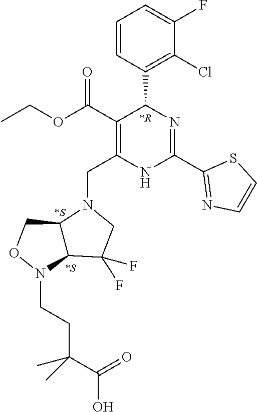 [SMILES: CCOC(=O)C1=C(CN2CC(F)(F)[C@@H]3[C@H]2CON3CCC(C)(C)C(=O)O)NC(c2nccs2)=N[C@H]1c1cccc(F)c1Cl] | 171 |
| 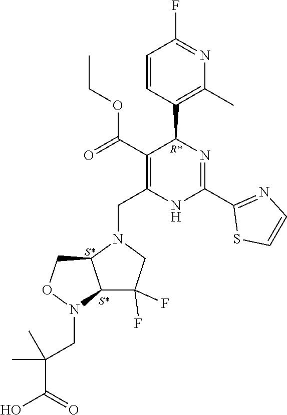 [SMILES: CCOC(=O)C1=C(CN2CC(F)(F)[C@@H]3[C@H]2CON3CC(C)(C)C(=O)O)NC(c2nccs2)=N[C@@H]1c1ccc(F)nc1C] | 172 |
| 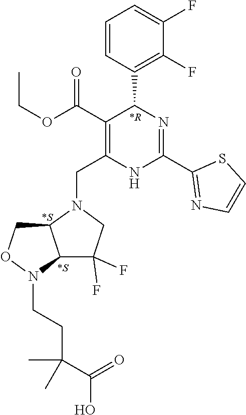 [SMILES: CCOC(=O)C1=C(CN2CC(F)(F)[C@@H]3[C@H]2CON3CCC(C)(C)C(=O)O)NC(c2nccs2)=N[C@H]1c1cccc(F)c1F] | 173 |
| 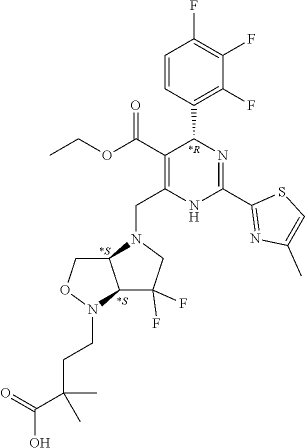 [SMILES: CCOC(=O)C1=C(CN2CC(F)(F)[C@@H]3[C@H]2CON3CCC(C)(C)C(=O)O)NC(c2nc(C)cs2)=N[C@H]1c1ccc(F)c(F)c1F] | 174 |
| 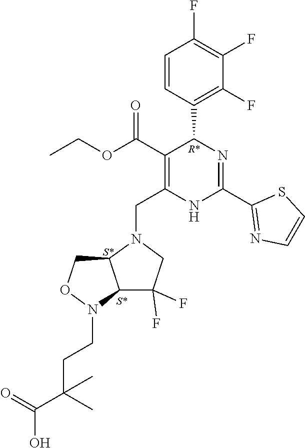 [SMILES: CCOC(=O)C1=C(CN2CC(F)(F)[C@@H]3[C@H]2CON3CCC(C)(C)C(=O)O)NC(c2nccs2)=N[C@H]1c1ccc(F)c(F)c1F] | 175 |
| 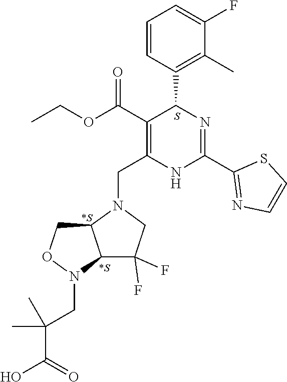 | 176 |
| 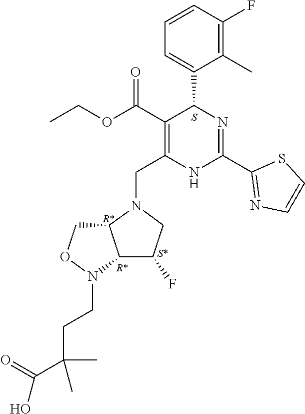 | 177 |
| 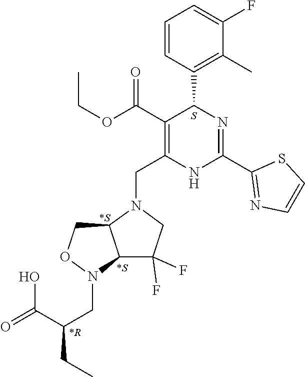 | 178A |
| 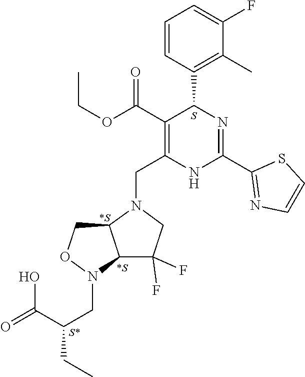 | 178B |
| 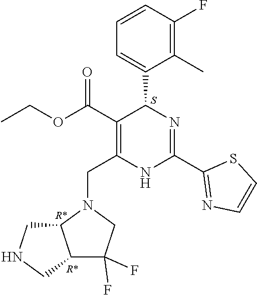 | 179 |
| 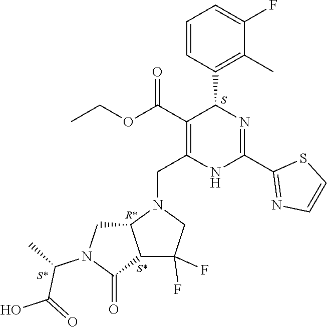 | 180 |
| 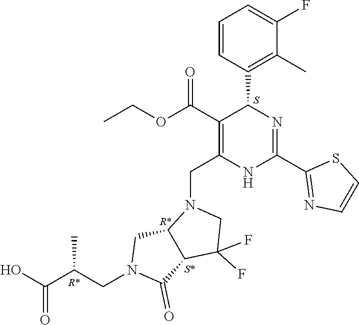 | 181A |
| 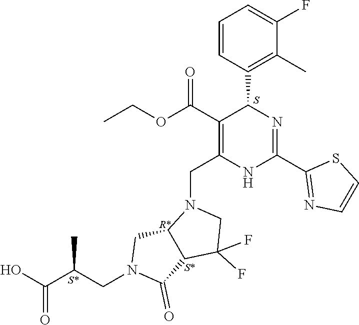 | 181B |
| 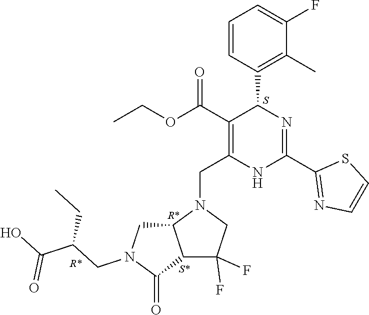 | 182A |
| 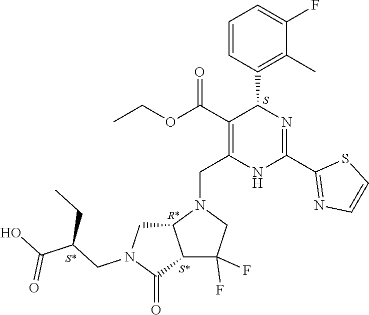 | 182B |
| 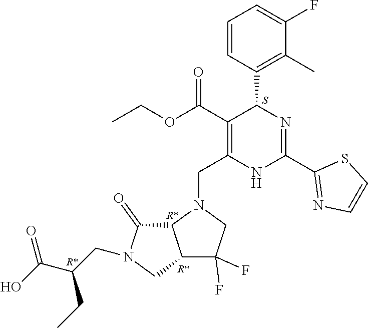 | 183A |
| 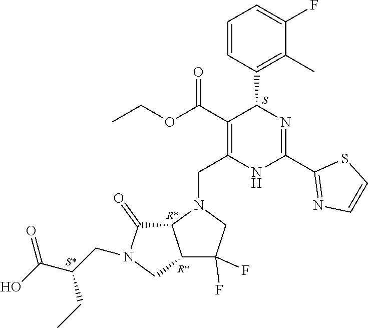 | 183B |
| 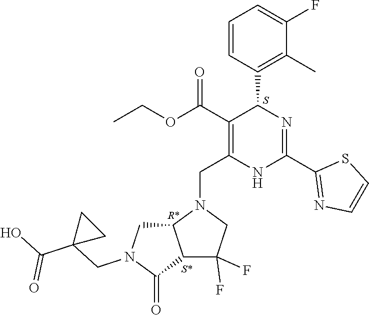 | 184 |
| 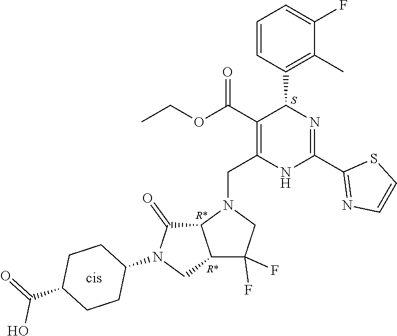 | 185A |
| 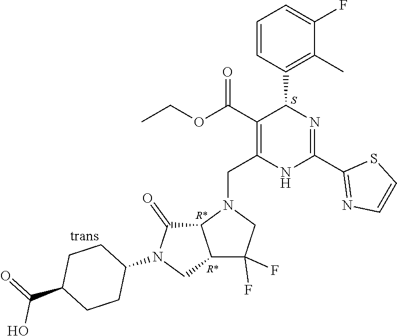 | 185B |
| 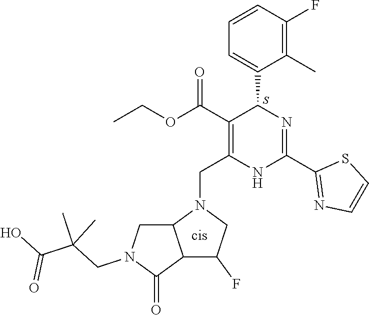 | 186 |
| 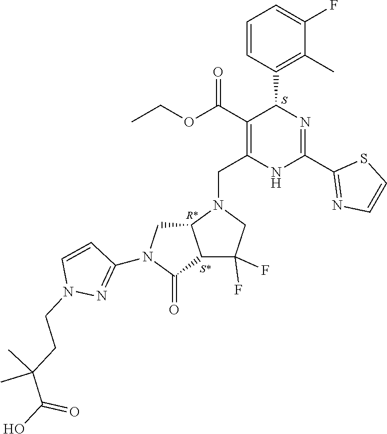 | 187 |
| 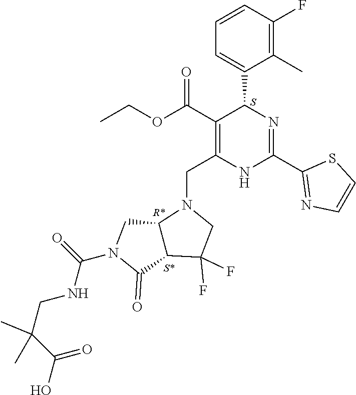 | 188 |
| 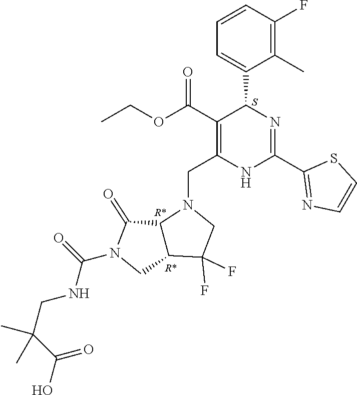 | 189 |
| 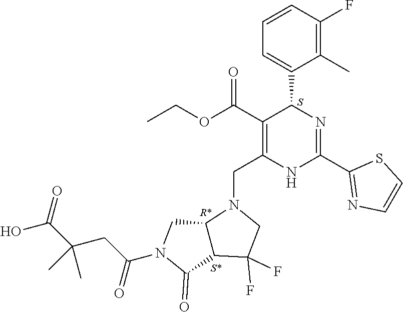 | 190 |
| 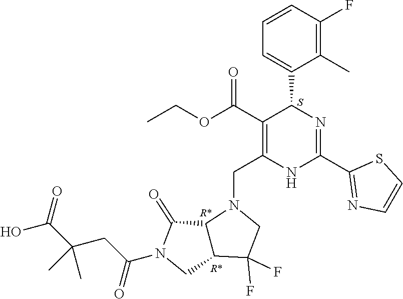 | 191 |
| 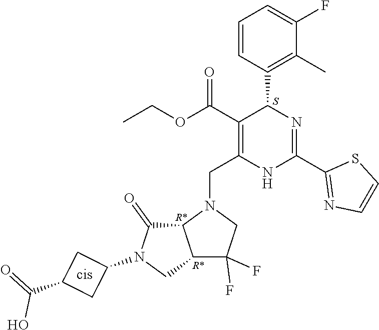 | 192 |
| 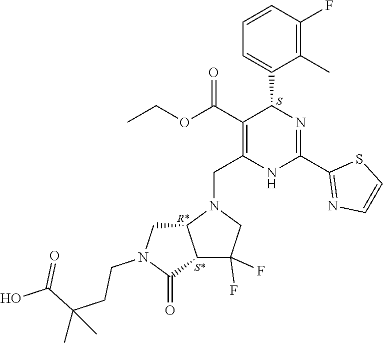 | 193 |
| 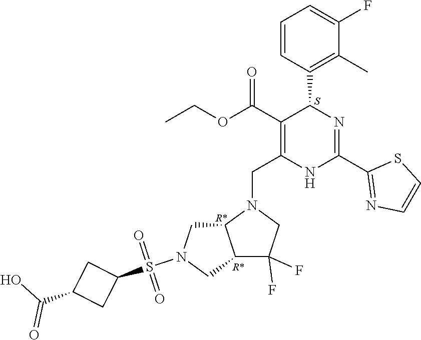 | 194 |
| 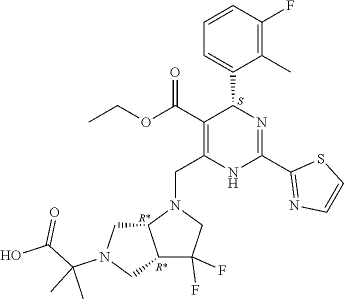 | 195 |
| 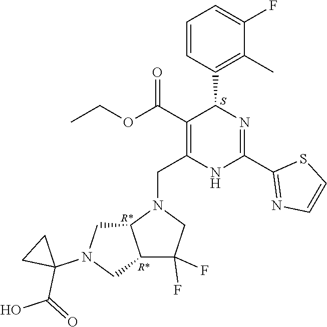 | 196 |
| 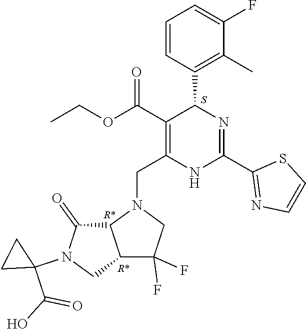 | 197 |
| 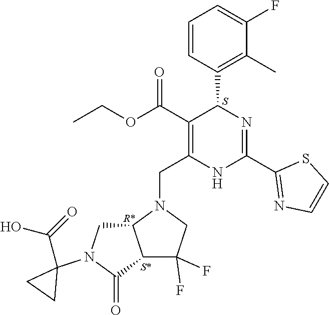 | 198 |
| 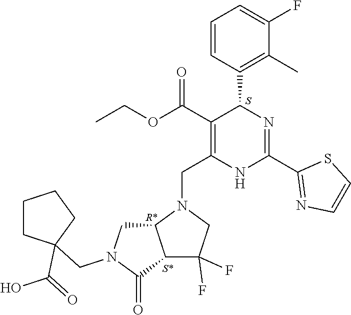 | 199 |
| 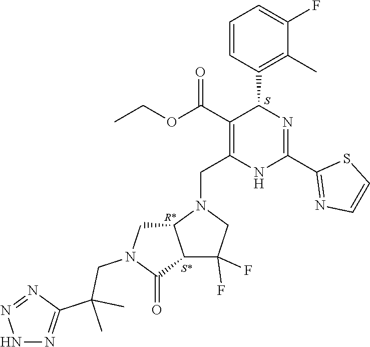 | 200 |
| 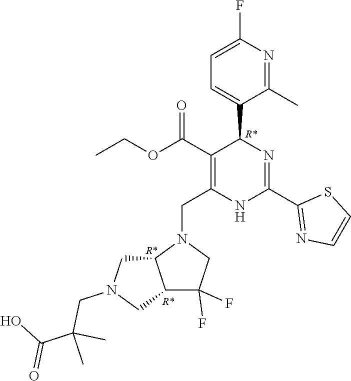 [SMILES: CCOC(=O)C1=C(CN2CC(F)(F)[C@@H]3CN(CC(C)(C)C(=O)O)C[C@@H]32)NC(c2nccs2)=N[C@@H]1c1ccc(F)nc1C] | 201 |
| 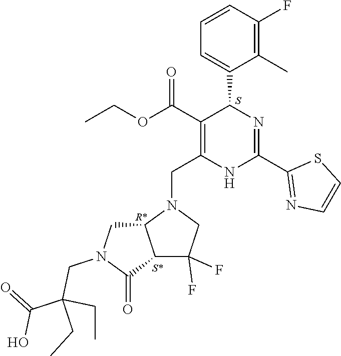 | 202 |
| 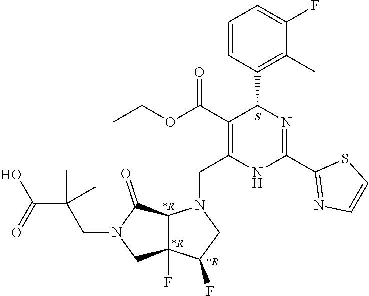 | 203A |
| 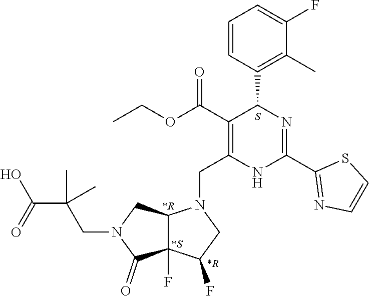 | 204A |
| 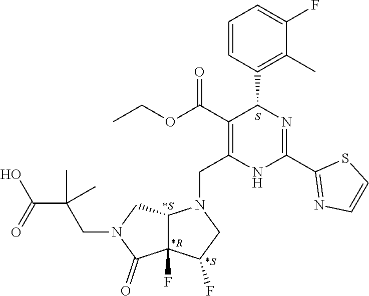 | 204B |
| 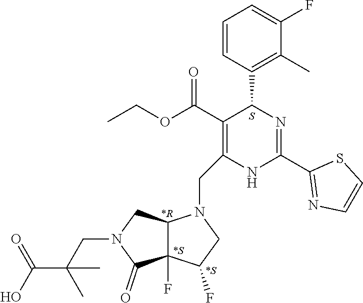 | 204C |
| 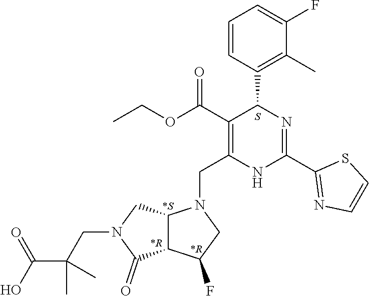 | 204D |
| 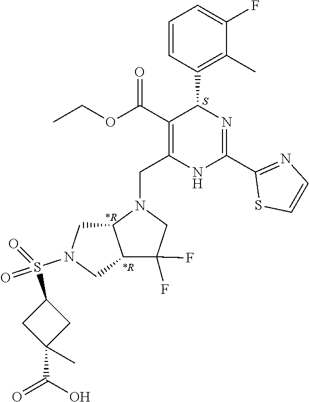 | 205A |
| 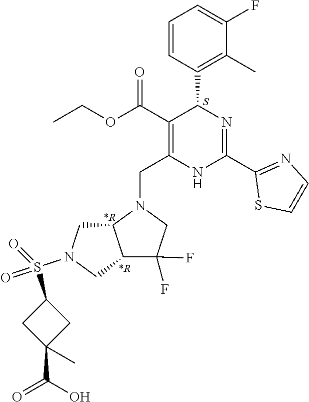 | 205B |
| 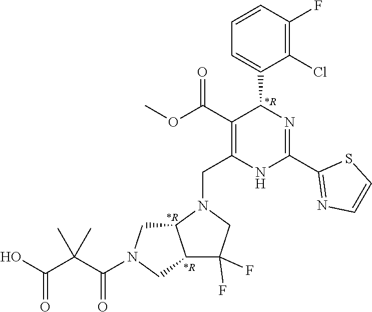 [SMILES: COC(=O)C1=C(CN2CC(F)(F)[C@@H]3CN(C(=O)C(C)(C)C(=O)O)C[C@@H]32)NC(c2nccs2)=N[C@H]1c1cccc(F)c1Cl] | 206 |
| 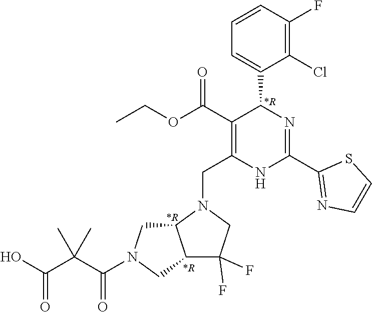 [SMILES: CCOC(=O)C1=C(CN2CC(F)(F)[C@@H]3CN(C(=O)C(C)(C)C(=O)O)C[C@@H]32)NC(c2nccs2)=N[C@H]1c1cccc(F)c1Cl] | 207 |
| 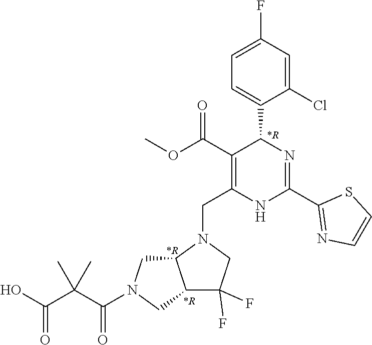 [SMILES: COC(=O)C1=C(CN2CC(F)(F)[C@@H]3CN(C(=O)C(C)(C)C(=O)O)C[C@@H]32)NC(c2nccs2)=N[C@H]1c1ccc(F)cc1Cl] | 208 |
| 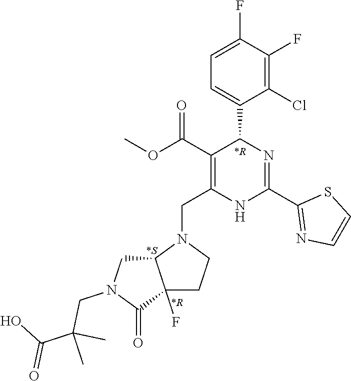 [SMILES: COC(=O)C1=C(CN2CC[C@]3(F)C(=O)N(CC(C)(C)C(=O)O)C[C@H]23)NC(c2nccs2)=N[C@H]1c1ccc(F)c(F)c1Cl] | 209A |
| 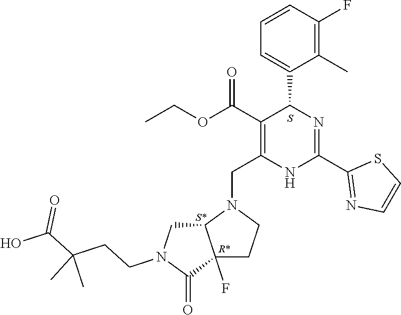 | 210B |
| 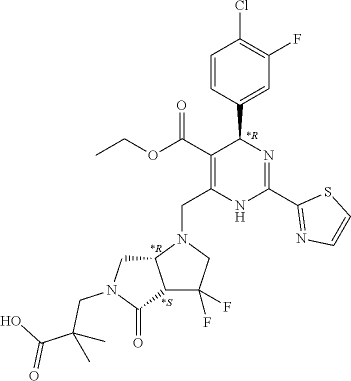 | 211 |

### Example 2: Anti-Viral Assay in HepG2.2.15 Cells

Materials and Equipments

1) Cell Line

HepG2.2.15 (the HepG2.2.15 cell line can be produced by transfection of the HepG2 cell line as described in Sells, Chen, and Acs 1987 (Proc. Natl. Acad. Sci. USA 84: 1005-1009), and the HepG2 cell line is available from ATCC® under number HIB-8065™)

2) Reagents

DMEM/F12 (INVITROGEN-11330032)

FBS (GIBCO-10099-141)

Dimethyl sulfoxide(DMSO) (SIGMA-D2650)

Penicillin-streptomycin solution (HYCLONE-SV30010)

NEAA (INVITROGEN-11114050)

L-Glutamine (INVITROGEN-25030081)

Geneticin Selective Antibiotic (G418, 500 mg/ml) (INVITROGEN-10131027)

Trypsinase digestion solution (INVITROGEN-25300062)

CCK8 (BIOLOTE-35004)

QIAamp 96 DNA Blood Kit (12) (QIAGEN-51162)

FastStart Universal Probe Mast Mix (ROCHE-04914058001)

3) Consumables

96-well cell culture plate (COSTAR-3599)

Micro Amp Optical 96-well reaction plate (APPLIED BIOSYSTEMS-4306737)

Micro Amp Optical 384-well reaction plate (APPLIED BIOSYSTEMS)

4) Equipment

Plate reader (MOLECULAR DEVICES, SPECTRAMAX M2e)

Centrifuge (BECKMAN, ALLEGRA-X15R)

Real Time PCR system (APPLIED BIOSYSTEMS, QUANTSTUDIO 6)

Real Time PCR system (APPLIED BIOSYSTEMS, 7900HT)

Methods

1) Anti-HBV Activity and Cytotoxicity Determination

HepG2.2.15 cells were plated into 96-well plate in 2% FBS culture medium at the density of 40,000 cells/well and 5,000 cells/well for HBV inhibitory activity and cytotoxicity determination, respectively. After incubation at 37° C., 5% CO2 overnight, cells were treated with medium containing compounds for 6 days with medium and compounds refreshed after 3 days of treatment. Each compound was tested in a 1:3 serial dilutions at 8 different concentrations in triplicate. The highest concentration of the compounds was 10 uM or 1 uM for anti-HBV activity assay and 100 uM for cytotoxicity determination.

Cell viability was determined by CCK-8 assay. After 6 days of compounds treatment, 20 μl CCK-8 reagents were added to each well of cytotoxicity assay plates. Cell plates were incubated at 37° C., 5% CO2 for 2.5 h. The absorbance at 450 nm wavelength and the absorbance at 630 nm wavelength as reference was measured.

The change of HBV DNA level induced by the compounds was assessed by quantitative real-time polymerase chain reaction (qPCR). Briefly, the HBV DNA in the culture medium was extracted using QIAamp 96 DNA Blood Kit according to the manual and then quantified by real-time PCR assay using the primers and probe in the table 2 below.

TABLE 2

| Primers | | SEQ |
|---|---|---|
| or Probe | Sequence | ID NO: |
| HBV-Fw | GTGTCTGCGGCGTTTTATCA | 1 |
| HBV-Rev | GACAAACGGGCAACATACCTT | 2 |
| HBV-Probe | CCTCTKCATCCTGCTGCTATGCCTCATC | 3 |
| With FAM | | |
| reporter | | |
| and TAMRA | | |
| quencher | | |

2) DATA Analysis EC₅₀ and CC₅₀ values are calculated by the GRAPTIPAD PRISM software. If the CV %0 of DMSO controls is below 1500 and the reference compounds shows expected activity or cytotoxicity, the data of this batch of experiment is considered qualified.

RESULTS: See Table 3 below.

TABLE 3

| Compound | EC50 | CC50 | Compound | EC50 | CC50 |
|---|---|---|---|---|---|
| No. | (μM) | (μM) | No. | (μM) | (μM) |
| 1 | 0.0106 | 24.8 | 112A | 0.0046 | 28.7 |
| 1A | 0.0013 | 15 | 112B | 0.0047 | 23.6 |
| 2 | 0.0041 | 26.7 | 113 | 0.0022 | 12.5 |
| 3 | 0.0202 | 63.1 | 113A | 0.0050 | >3 |
| 3B | 0.0387 | 51.8 | 113B | >0.001 | >3.1 |
| 4 | 0.0564 | 47.8 | 114 | 0.0055 | 34.4 |
| 4A | 0.0063 | 32.6 | 115 | 0.0031 | 24.0 |
| 5 | 0.0227 | 90.1 | 116 | 0.0007 | 14.3 |
| 5A | 0.0082 | 70.4 | 116A | 0.0008 | 12.1 |
| 6 | 0.0104 | 37.2 | 116B | 0.0025 | 10.6 |
| 6A | 0.0036 | 30.4 | 117 | <0.0005 | 24.7 |
| 6B | 0.3824 | 34.7 | 118 | 0.0054 | 12.7 |
| 7B | 0.0008 | 20.4 | 119 | 0.0555 | 20.2 |
| 8A | 0.0069 | 36.3 | 120A | 0.0120 | 28.1 |
| 9 | 0.0417 | 76.3 | 120B | 0.0130 | 19.9 |
| 9A | 0.0111 | 57.8 | 121 | 0.0087 | >100 |
| 10 | 0.0805 | 69.6 | 122A | 0.0060 | >100 |
| 11 | 0.0703 | 37.3 | 123A | 0.0080 | 18.6 |
| 11B | 0.0314 | 30 | 123B | 0.0090 | 16.1 |
| 12 | 0.0324 | 56.7 | 124 | 0.0242 | 7.7 |
| 12B | 0.0082 | 86.1 | 125 | 0.0093 | 58.4 |
| 13 | 0.0084 | 50 | 126 | 0.0034 | 27.2 |
| 13B | 0.0035 | 38.6 | 127 | 0.0016 | 47.2 |
| 14 | 0.1372 | >100 | 128 | 0.0008 | 40.1 |
| 14B | 0.1018 | 67.9 | 129A | 0.0077 | 10.3 |
| 14E | 0.0623 | 85.3 | 129B | 0.0029 | 12.4 |
| 14F | 0.1254 | >100 | 130A | 0.0076 | 22.4 |
| 15 | 0.0603 | >100 | 130B | <0.0005 | 18.2 |
| 15B | 0.0272 | >100 | 131A | 0.0005 | 44.6 |
| 15E | 0.0225 | 41.7 | 131B | 0.0297 | 65.6 |
| 15F | 0.0161 | 39 | 132A | <0.0005 | 40.7 |
| 16 | 0.1118 | 37.2 | 132B | 0.0107 | 71.8 |
| 16B | 0.0686 | >100 | 133A | <0.0005 | 41.7 |
| 17B | 0.1232 | 72.8 | 134A | <0.0005 | 26.9 |
| 18A | 0.0611 | 66.3 | 134B | 0.0043 | 24.2 |
| 18B | 0.0737 | 64.2 | 135A | 0.0024 | 25.9 |
| 19B | 0.0493 | 24.2 | 135B | 0.0099 | 36.3 |
| 20B | 0.0395 | >100 | 136 | 0.0021 | 34.4 |
| 21B | 0.0227 | 97.3 | 137 | 0.0010 | 21.8 |
| 22B | 0.0089 | 70.4 | 138 | 0.0005 | 14.5 |
| 23B | 0.024 | 30.2 | 139 | 0.0006 | 16.1 |
| 24A | 0.2212 | 48.5 | 140 | 0.0070 | 36.7 |
| 25 | 0.0952 | 95.5 | 141 | 0.0035 | 26.7 |
| 25A | 0.0324 | 74.5 | 142 | 0.0080 | >25 |
| 26E | 0.0052 | 28.1 | 143A | 0.0011 | 19.2 |
| 26F | 0.0148 | 41.7 | 143B | 0.0046 | 22.8 |
| 27 | 0.042 | 13.9 | 144 | 0.0198 | 20.2 |
| 28 | 0.0282 | >100 | 145 | 0.0009 | 26.0 |
| 29 | 0.0135 | >100 | 146 | 0.0131 | 32.2 |
| 30 | 0.0813 | 28 | 147 | <0.0005 | 34.6 |
| 31 | 0.1059 | 85.8 | 148 | 0.0073 | 34.1 |
| 32 | 0.0034 | 31.8 | 149A | <0.0005 | 21.8 |
| 33 | 0.0024 | 14.6 | 149B | 0.0019 | 34.1 |
| 34 | 0.012 | 14.3 | 150 | 0.0570 | 15.0 |
| 35 | 0.0133 | 35.2 | 151 | 0.0187 | 9.3 |
| 36 | 0.0301 | 26 | 152 | 0.0618 | 12.7 |
| 38A | 0.0015 | 20 | 153 | 0.0060 | 21.8 |
| 39A | 0.0024 | 35 | 153A | 0.0022 | 11.8 |
| 41 | 0.0938 | >100 | 153B | 0.0158 | 15.3 |
| 42 | 0.0138 | >100 | 154 | 0.0710 | 48.3 |
| 43 | 0.0041 | 50.2 | 155 | 0.0007 | 10.0 |
| 44A | 0.0567 | >100 | 156 | 0.0630 | 10.3 |
| 46 | 0.0394 | 20.9 | 157 | 0.0008 | 24.8 |
| 48 | 0.1049 | >100 | 158A | 0.0531 | 27.9 |
| 49 | 0.0326 | 25.5 | 158B | 0.0526 | 16.6 |
| 50A | 0.0408 | 49.3 | 159 | 0.0006 | 75.7 |
| 54 | 0.0492 | 55.2 | 160 | 0.0140 | 100.0 |
| 56B | 0.0792 | >100 | 161 | 0.0080 | >50 |
| 57B | 0.0922 | 51.2 | 162 | 0.0050 | 50.0 |
| 60 | 0.0496 | 29.4 | 163 | 0.0020 | 45.6 |
| 61 | 0.0373 | 21.5 | 164 | 0.0030 | >25 |
| 62 | 0.0127 | 17.3 | 165 | 0.0080 | >50 |
| 63 | 0.0118 | 25.2 | 166 | 0.0590 | >100 |
| 68A | 0.0074 | 22.2 | 167 | 0.0163 | >100 |
| 69A | 0.0007 | 37 | 168 | 0.0090 | >100 |
| 70 | 0.0081 | 27.1 | 169 | 0.0050 | >50 |
| 70A | 0.0350 | >50 | 170 | 0.0080 | >25 |
| 70B | 0.0160 | >50 | 171 | 0.0040 | 32.3 |
| 71 | 0.0290 | 20.9 | 172 | 0.0330 | >100 |
| 72A | 0.0550 | 14.5 | 173 | 0.0160 | >100 |
| 72B | 0.0240 | 16.9 | 174 | 0.0150 | >50 |
| 73 | 0.0170 | 26.0 | 175 | 0.0080 | >50 |
| 74 | 0.0380 | 16.6 | 176 | 0.0030 | 24.8 |
| 75 | 0.0400 | 22.6 | 177 | 0.0150 | 24.0 |
| 76A | 0.0110 | 28.2 | 178A | 0.0035 | >50 |
| 76B | 0.0905 | 68.8 | 178B | 0.0120 | >50 |
| 77B | 0.0150 | 17.5 | 179 | 0.0500 | >3.1 |
| 78 | 0.0490 | 70.3 | 180 | 0.0926 | >100 |
| 79A | 0.0400 | >50 | 181A | 0.0123 | 52.9 |
| 79B | 0.0270 | 54.9 | 181B | 0.0095 | 69.9 |
| 80A | 0.0155 | 38.2 | 182A | 0.0239 | 49.6 |
| 80B | 0.0080 | 24.1 | 182B | 0.0113 | 33.3 |
| 81A | 0.0165 | 60.6 | 183A | 0.0143 | 40.3 |
| 81B | 0.0130 | 35.9 | 183B | 0.0064 | 50.2 |
| 82B | 0.0370 | 46.9 | 184 | 0.0067 | 48.3 |
| 83B | 0.0350 | 39.0 | 185A | 0.0054 | 48.3 |
| 83A | 0.0460 | 73.5 | 185B | 0.0083 | 27.6 |
| 84A | 0.0520 | >100 | 186 | 0.0089 | 66.9 |
| 85 | 0.0690 | 22.6 | 186A | 0.0460 | >100 |
| 86 | 0.1300 | >50 | 187 | 0.0817 | 33.6 |
| 87 | 0.0140 | >50 | 188 | 0.0624 | 32.1 |
| 88 | 0.0015 | 60.0 | 189 | 0.0181 | 28.2 |
| 89 | 0.0600 | >3.12 | 190 | 0.0043 | 28.7 |
| 90 | 0.0205 | 19.2 | 191 | 0.0135 | 35.9 |
| 91 | 0.0710 | 8.8 | 192 | 0.0166 | 52.9 |
| 92 | 0.0610 | 5.0 | 193 | 0.0121 | 48.2 |
| 93 | 0.0120 | 5.4 | 194 | 0.0713 | 36.7 |
| 94 | 0.0140 | 25.0 | 195 | 0.0403 | 96.6 |
| 95 | 0.0240 | 8.2 | 196 | 0.0033 | 12.8 |
| 96 | 0.0500 | >100 | 197 | 0.0142 | 26.0 |
| 97 | 0.0770 | >100 | 198 | 0.0960 | 99.6 |
| 98 | 0.0140 | >6.2 | 199 | 0.0118 | 35.9 |
| 99 | 0.1550 | 25.0 | 200 | 0.0347 | 91.3 |
| 100 | 0.0110 | 12.0 | 201 | 0.0726 | >100 |
| 101 | 0.0250 | >3.1 | 202 | 0.0230 | 100.0 |
| 102 | 0.0135 | 53.8 | 203A | 0.0167 | 44.6 |
| 103 | 0.0543 | 19.8 | 204A | 0.0112 | 88.9 |
| 104A | 0.0104 | 16.1 | 204B | 0.1021 | 72.1 |
| 104B | 0.0080 | >6.2 | 204C | 0.0071 | 36.9 |
| 105A | 0.0105 | 15.2 | 204D | 0.0004 | 48.1 |
| 105B | 0.1080 | 94.3 | 205A | 0.1140 | >50 |
| 106 | 0.0200 | 39.5 | 205B | 0.1030 | >50 |
| 107 | 0.0113 | 42.5 | 206 | 0.0393 | >100 |
| 108 | 0.0004 | 13.1 | 207 | 0.0590 | >50 |
| 109 | >0.001 | 7.3 | 208 | 0.0286 | >100 |
| 110 | 0.0006 | 23.5 | 209A | 0.0191 | 23.3 |
| 111 | 0.0078 | 33.4 | 210B | 0.0977 | 48.7 |
| 112 | 0.0053 | 28.6 | 211 | 0.0360 | >50 |

1. A compound of Formula (I) 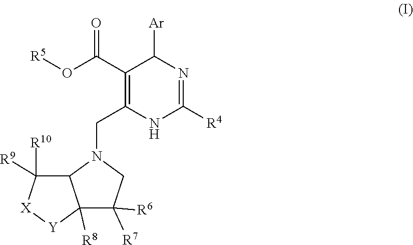
or a deuterated form, stereoisomer or tautomer thereof, wherein:
Ar is selected from the group consisting of phenyl, thiophenyl, and pyridyl, optionally substituted with one or more substituents selected from the group consisting of C₁₋₄alkyl, hydroxyl, halogen, and CN;
R⁴ is selected from the group consisting of thiazolyl, imidazolyl, oxazolyl and pyridyl, each of which is optionally substituted with one or more substituents, each independently selected from methyl or halo;
R⁵ is C₁₋₄alkyl;
R⁶, R⁷ and R⁸ are each independently selected from the group consisting of H and halo;
R⁹ and R¹⁰ are each independently selected from the group consisting of H, halo and OH; or
R⁹ and R¹⁰, together with the carbon atom to which they are attached, form C(═O);
X is selected from the group consisting of CH₂, C(═O), O, S, S(═O), S(═O)₂, NH, NR^{11a}, CHR^{12a}, and CR¹⁵R¹⁶; and
Y is selected from the group consisting of CH₂, C(═O), O, NH, NR^{11b} and CHR^{12b}; wherein
R^{11a}, R^{11b}, R^{12a}, and R^{12b} are each independently selected from the group consisting of —CN; —C₁₋₆alkyl, —COOR^{x}; —C₁₋₉alkyl-COOR^{x}; —C₁₋₆alkyl-O—C₁₋₆alkyl-COOR^{x}; -Cy-COOR^{x}; —C₁₋₆alkyl-C(═O)—NR—S(═O)₂—C₁₋₆alkyl; —C₁₋₆alkyl-Cy-COOR^{x}; -Cy-C₁₋₆alkyl-COOR^{x}; —C₁₋₆alkyl-Cy-C₁₋₆alkyl-COOR^{x}; —C(═O)—C₁₋₆alkyl; —C(═O)—C₁₋₆alkyl-COOR^{x}; —C(═O)—Cy-COOR^{x}; —C(═O)—O—C₁₋₆alkyl-COOR^{x}; —C(═O)—C₁₋₆alkyl-O—C₁₋₆alkyl-COOR^{x}; —C(═O)H; —C(═O)—NR^{a}R^{b}; —C(═O)-Het¹; —C(═O)—Cy; —C(═O)—NR^{c}—C₁₋₆alkyl-COOR^{x}; —C(═O)—C₁₋₆alkyl-NR^{c}—C₁₋₆alkyl-COOR^{x}; —C(═O)—NR^{c}—COOR^{x}; —C(═O)—NR^{c}—CO—NR^{a}R^{b}; —C(═O)—NR-Cy-COOR^{x}; —C(═O)—NR^{c}—S(═O)₂—C₁₋₆alkyl; —C(═O)-Het¹-COOR^{x}; —C(═O)—NR^{c}-Het¹-COOR^{x}; —C(═O)—C(═O)—NR^{a}R^{b}; —C(═O)—C(═O)-Het¹; —C(═O)—C(═O)—O—C₂₋₆alkenyl; -Het¹-COOR^{x}; -Het¹-C₁₋₆alkyl-COOR^{x}; —C₁₋₆alkyl-Het¹-COOR^{x}; —C₁₋₆alkyl-Het¹-C₁₋₆alkyl-COOR^{x}; —C₁₋₆alkyl-C(═O)-Het¹-COOR^{x}; -Het²-COOR^{x}; —C₁₋₆alkyl-Het²; —C₁₋₆alkyl-Het²-COOR^{x}; -Het²-C₁₋₆alkyl-COOR^{x}; —C₁₋₆alkyl-Het²-C₁₋₆alkyl-COOR^{x}; —NR^{c}—C₁₋₆alkyl-COOR^{x}; —NR^{c}—Cy-COOR^{x}; —NR^{c}—Het¹-COOR^{x}; —O—C₁₋₉alkyl-COOR^{x}; —S(═O)₂—NR^{a}R^{b}; —S(═O)₂—C₁₋₆alkyl; —S(═O)₂—C₁₋₆alkyl-COOR^{x}; —S(═O)₂-Cy-COOR^{x}; —S(═O)₂-Cy-C₁₋₆alkyl-COOR^{x}; —S(═O)₂—NR^{c}-Cy-COOR^{x}; —S(═O)₂—NR^{c}-Het²; —S(═O)₂-Het¹-COOR^{x}; —S(═O)₂-Het¹-C₁₋₆alkyl-COOR^{x}; —S(═O)₂—NR^{c}—C(═O)—C₁₋₆alkyl; —C(═O)—NR^{c}—S(═O)₂—C₁₋₆alkyl; and —C₁₋₆alkyl-C(═O)—NR^{c}—S(═O)₂-Ci-alkyl;
wherein
R^{a}, R^{b} and R^{c} are each independently selected from H and —C₁₋₄alkyl;
at each instance, the C₁₋₆alkyl and C₁₋₉alkyl is optionally substituted with one or more substituents, each independently selected from halo and hydroxyl;
R^{x} is selected from H and —C₁₋₆alkyl;
Cy is selected from C₃₋₇cycloalkyl and 5- to 11-membered bicyclic saturated carbocyclyl, each optionally substituted with one or more substituents selected from halo and —C₁₋₄alkyl;
Het¹ represents a 4- to 8-membered saturated ring in which 1 or 2 of the ring members is a heteroatom each independently selected from the group consisting of N, O, and S; wherein the 4- to 8-membered saturated ring is optionally substituted with one or more substituents, each independently selected from C₁₋₄alkyl and OH; and
Het² represents a 5- to 6-membered aromatic ring in which 1, 2, 3 or 4 of the ring members is a heteroatom each independently selected from N, O, or S; wherein the 5- to 6-membered aromatic ring is optionally substituted with one or more substituents, each independently selected from C₁₋₄alkyl and halo;
with the proviso that CR⁹R¹⁰ and X, or X and Y, are not simultaneously both C(═O);
R¹⁵ and R¹⁶, together with the carbon atom to which they are attached, form a C₃₋₇cycloalkyl, optionally substituted with one or more substituents selected from halo and —C₁₋₄alkyl;
or a pharmaceutically acceptable salt or a solvate thereof.

2. The compound according to claim 1, wherein the compound is of Formula (II) 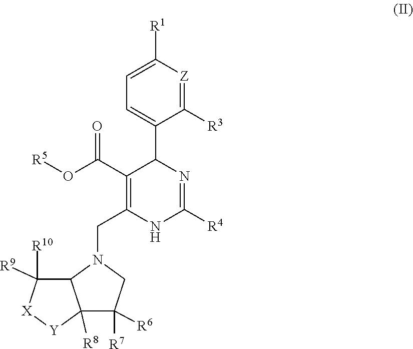
wherein
Z is N or CR²;
R¹, R² and R³ are each independently selected from the group consisting of H, halo, OH, and C₁₋₃alkyl.

3. The compound according to claim 2, wherein
Z is CR²;
R¹, R² and R³ are each independently selected from the group consisting of H, halo, and C₁₋₃alkyl;
R⁴ is selected from the group consisting of thiazolyl, imidazolyl, and oxazolyl, each of which is optionally substituted with one or more methyl substituents;
R⁵ is C₁₋₄alkyl;
R⁶, R⁷ and R⁸ are each independently selected from the group consisting of H and halo;
R⁹ and R¹⁰ are each independently selected from the group consisting of H and halo; or
R⁹ and R¹⁰, together with the carbon atom to which they are attached, form C(═O);
X is selected from the group consisting of CH₂, C(═O), O, NH, NR^{11a} and CHR^{12a}; and
Y is selected from the group consisting of CH₂, C(═O), O, NH, NR^{11b} and CHR^{12b}; wherein
R^{11a}, R^{11b}, R^{12a}, and R^{12b} are each independently selected from the group consisting of —CN; —C₁₋₆alkyl, —COOR^{x}; —C₁₋₆alkyl-C(═O)—NR^{c}—S(═O)₂—C₁₋₆alkyl; —C₁₋₉alkyl-COOR^{x}, in particular —C₁₋₆alkyl-COOR^{x}; —C₁₋₆alkyl-O—C₁₋₆alkyl-COOR^{x}; -Cy-COOR^{x}; —C₁₋₆alkyl-Cy-COOR^{x}; —C₁₋₆alkyl-Cy-C₁₋₆alkyl-COOR^{x}; —C(═O)—C₁₋₆alkyl; —C(═O)—C₁₋₆alkyl-COOR^{x}; —C(═O)—Cy-COOR^{x}; —C(═O)—O—C₁₋₆alkyl-COOR^{x}; —C(═O)—C₁₋₆alkyl-O—C₁₋₆alkyl-COOR^{x}; —C(═O)H; —C(═O)—NR^{a}R^{b}; —C(═O)-Het¹; —C(═O)—Cy; —C(═O)—NR^{c}—C₁₋₆alkyl-COOR^{x}; —C(═O)—C₁₋₆alkyl-NR^{c}—C₁₋₆alkyl-COOR^{x}; —C(═O)—NR^{c}—CO—NR^{a}R^{b}; —C(═O)—NR^{c}-Cy-COOR^{x}; —C(═O)—NR^{c}—S(═O)₂—C₁₋₆alkyl; —C(═O)—C(═O)-Het¹; —C(═O)—C(═O)—O—C₂₋₆alkenyl; -Het¹-C₁₋₆alkyl-COOR^{x}; —C₁₋₆alkyl-C(═O)-Het¹-COOR^{x}; -Het²-COOR^{x}; —C₁₋₆alkyl-Het²; —C₁₋₆alkyl-Het²-COOR^{x}; -Het²-C₁₋₆alkyl-COOR^{x}; —C₁₋₆alkyl-Het²-C₁₋₆alkyl-COOR^{x}; —NR^{c}—C₁₋₆alkyl-COOR^{x}; —O—C₁₋₉alkyl-COOR^{x}, in particular —O—C₁₋₆alkyl-COOR^{x}; —S(═O)₂—NR^{a}R^{b}; —S(═O)₂—C₁₋₆alkyl; —S(═O)₂—C₁₋₆alkyl-COOR^{x}; —S(═O)₂-Cy-COOR^{x}; —S(═O)₂—NR^{c}-Cy-COOR^{x}; —S(═O)₂—NR^{c}-Het²; —S(═O)₂-Het¹-COOR^{x}; —S(═O)₂—NR^{c}—C(═O)—C₁₋₆alkyl; —C(═O)—NR^{c}—S(═O)₂—C₁₋₆alkyl; and —C₁₋₆alkyl-C(═O)—NR^{c}—S(═O)₂—C₁₋₆alkyl.

4. The compound according to claim 1, wherein
R^{11a}, R^{11b}, R^{12a}, and R^{12b} are each independently selected from the group consisting of —CN; —C₁₋₆alkyl, —COOH; —C₁₋₉alkyl-COOH, in particular —C₁₋₆alkyl-COOH; -Cy-COOH; —C₁₋₆alkyl-Cy-COOH; —C₁₋₆alkyl-Cy-C₁₋₆alkyl-COOH; —C(═O)—C₁₋₆alkyl; —C(═O)—C₁₋₆alkyl-COOH; —C(═O)—Cy-COOH; —C(═O)—O—C₁₋₆alkyl-COOH; —C(═O)—C₁₋₆alkyl-O—C₁₋₆alkyl-COOH; —C(═O)—NR^{a}R^{b}; —C(═O)-Het¹; —C(═O)—NR^{c}—C₁₋₆alkyl-COOH; —C(═O)—C₁₋₆alkyl-NR^{c}—C₁₋₆alkyl-COOH; —C(═O)—NR^{c}—CO—NR^{a}R^{b}; —C(═O)—NR^{c}-Cy-COOH; —C(═O)—C(═O)-Het¹; —C(═O)—C(═O)—O—C₂₋₆alkenyl; -Het¹-C₁₋₆alkyl-COOH; —C₁₋₆alkyl-C(═O)-Het¹-COOH; -Het²-COOH; —S(═O)₂—NR^{a}R^{b}; —S(═O)₂—C₁₋₆alkyl; —S(═O)₂—C₁₋₆alkyl-COOH; —S(═O)₂—NR^{c}—C(═O)—C₁₋₆alkyl; —C(═O)—NR^{c}—S(═O)₂—C₁₋₆alkyl; and —C₁₋₆alkyl-C(═O)—NR^{c}—S(═O)₂—C₁₋₆alkyl.

5. The compound according to claim 1, wherein
X is selected from the group consisting of CH₂, O, NR^{11a}, and CHR^{12a};
Y is selected from the group consisting of CH₂, C(═O), NR^{11b} and CHR^{12b};
R^{11a} is selected from the group consisting of
—C₁₋₉alkyl-COOH; —C₁₋₆alkyl-O—C₁₋₆alkyl-COOH; -Cy-COOH; —C₁₋₆alkyl-C(═O)—NR^{c}—S(═O)₂—C₁₋₆alkyl; —C₁₋₆alkyl-Cy-COOH; -Cy-C₁₋₆alkyl-COOH; —C₁₋₆alkyl-Cy-C₁₋₆alkyl-COOH; —C(═O)—C₁₋₆alkyl; —C(═O)—C₁₋₆alkyl-COOH; —C(═O)—Cy-COOH; —C(═O)—O—C₁₋₆alkyl-COOH; —C(═O)—C₁₋₆alkyl-O—C₁₋₆alkyl-COOH; —C(═O)H; —C(═O)—NR^{a}R^{b}; —C(═O)—Cy; —C(═O)—NR^{c}—C₁₋₆alkyl-COOH; —C(═O)—C₁₋₆alkyl-NR^{c}—C₁₋₆alkyl-COOH; —C(═O)—NR^{c}—COOH; —C(═O)—NR^{c}-Cy-COOH; —C(═O)—NR^{c}—S(═O)₂—C₁₋₆alkyl; —C(═O)-Het¹-COOH; —C(═O)—NR^{c}-Het¹-COOH; —C(═O)—C(═O)—NR^{a}R^{b}; -Het¹-COOH; -Het¹-C₁₋₆alkyl-COOH; —C₁₋₆alkyl-Het¹-COOH; —C₁₋₆alkyl-Het¹-C₁₋₆alkyl-COOH; -Het²-COOH; —C₁₋₆alkyl-Het²; —C₁₋₆alkyl-Het²-COOH; -Het²-C₁₋₆alkyl-COOH; —C₁₋₆alkyl-Het²-C₁₋₆alkyl-COOH; —S(═O)₂—C₁₋₆alkyl-COOH; —S(═O)₂-Cy-COOH; —S(═O)₂-Cy-C₁₋₆alkyl-COOH; —S(═O)₂-Het¹-COOH; —S(═O)₂-Het¹-C₁₋₆alkyl-COOH; —S(═O)₂—NR^{c}—C(═O)—C₁₋₆alkyl; —C(═O)—NR^{c}—S(═O)₂—C₁₋₆alkyl; and —C₁₋₆alkyl-C(═O)—NR^{c}—S(═O)₂—C₁₋₆alkyl;
R^{12a} is selected from the group consisting of —C₁₋₆alkyl, and —COOH;
R^{11b} and R^{12b} are independently selected from the group consisting of
—C₁₋₉alkyl-COOH; —C₁₋₆alkyl-O—C₁₋₆alkyl-COOH; -Cy-COOH; —C₁₋₆alkyl-C(═O)—NR^{c}—S(═O)₂—C₁₋₆alkyl; —C₁₋₆alkyl-Cy-COOH; -Cy-C₁₋₆alkyl-COOH; —C₁₋₆alkyl-Cy-C₁₋₆alkyl-COOH; —C(═O)—C₁₋₆alkyl; —C(═O)—C₁₋₆alkyl-COOH; —C(═O)—Cy-COOH; —C(═O)—O—C₁₋₆alkyl-COOH; —C(═O)—C₁₋₆alkyl-O—C₁₋₆alkyl-COOH; —C(═O)—NR^{a}R^{b}; —C(═O)—Cy; —C(═O)—NR^{c}—C₁₋₆alkyl-COOH; —C(═O)—C₁₋₆alkyl-NR^{c}—C₁₋₆alkyl-COOH; —C(═O)—NR^{c}—COOH; —C(═O)—NR^{c}—CO—NR^{a}R^{b}; —C(═O)—NR^{c}-Cy-COOH; —C(═O)—NR^{c}—S(═O)₂—C₁₋₆alkyl; —C(═O)-Het¹-COOH; —C(═O)—NR^{c}-Het¹-COOH; -Het¹-COOH; -Het¹-C₁₋₆alkyl-COOH; —C₁₋₆alkyl-Het¹-COOH; —C₁₋₆alkyl-Het¹-C₁₋₆alkyl-COOH; —C₁₋₆alkyl-C(═O)-Het¹-COOH; -Het²-COOH; —C₁₋₆alkyl-Het²; —C₁₋₆alkyl-Het²-COOH; -Het²-C₁₋₆alkyl-COOH; —C₁₋₆alkyl-Het²-C₁₋₆alkyl-COOH; —O—C₁₋₉alkyl-COOH; —S(═O)₂—NR^{a}R^{b}; —S(═O)₂—C₁₋₆alkyl; —S(═O)₂—C₁₋₆alkyl-COOH; —S(═O)₂-Cy-COOH; —S(═O)₂-Cy-C₁₋₆alkyl-COOH; —S(═O)₂—NR^{c}-Cy-COOH; —S(═O)₂—NR^{c}-Het²; —S(═O)₂-Het¹-COOH; —S(═O)₂-Het¹-C₁₋₆alkyl-COOH; —C(═O)—NR^{c}—S(═O)₂—C₁₋₆alkyl; and —C₁₋₆alkyl-C(═O)—NR^{c}—S(═O)₂—C₁₋₆alkyl.

6. The compound according to claim 1, wherein R¹, R² and R³ are each independently selected from the group consisting of H, halo, OH, and methyl.

7. The compound according to claim 1, wherein R⁴ is selected from the group consisting of thiazolyl, imidazolyl, oxazolyl and pyridyl, each of which is optionally substituted with one methyl substituent.

8. The compound according to claim 1, wherein R⁵ is methyl or ethyl.

9. The compound according to claim 1, wherein R⁶, R⁷ and R⁸ are each independently selected from hydrogen and halo.

10. The compound according to claim 1, wherein R⁹ and R¹⁰ are each independently selected from hydrogen and halo; or R⁹ and R¹⁰, together with the carbon atom to which they are attached, form C(═O).

11. A compound selected from the group consisting of the compounds having the following formulae: 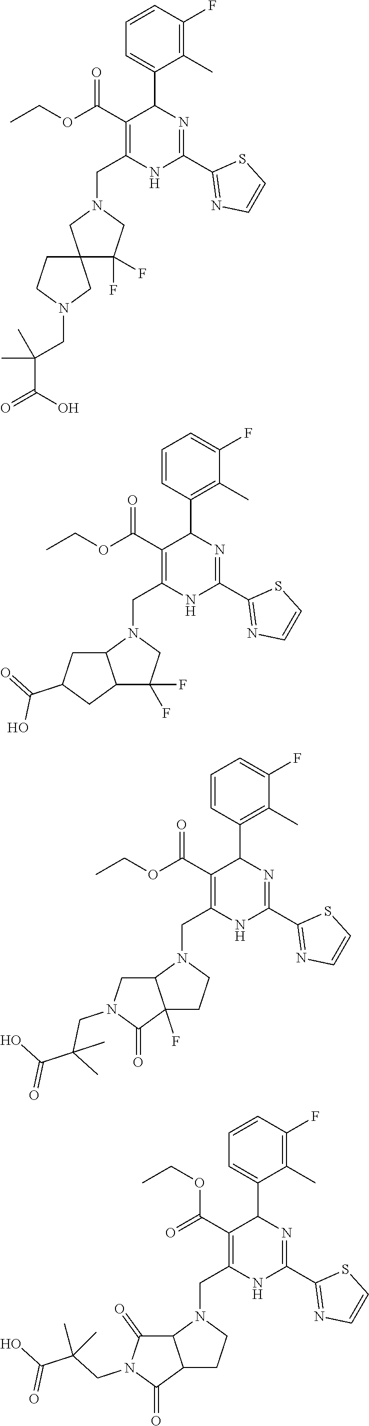 [SMILES: CCOC(=O)C1=C(CN2CC(F)(F)C3(CCN(CC(C)(C)C(=O)O)C3)C2)NC(c2nccs2)=NC1c1cccc(F)c1C ; CCOC(=O)C1=C(CN2CC(F)(F)C3CC(C(=O)O)CC32)NC(c2nccs2)=NC1c1cccc(F)c1C ; CCOC(=O)C1=C(CN2CCC3(F)C(=O)N(CC(C)(C)C(=O)O)CC23)NC(c2nccs2)=NC1c1cccc(F)c1C ; CCOC(=O)C1=C(CN2CCC3C(=O)N(CC(C)(C)C(=O)O)C(=O)C32)NC(c2nccs2)=NC1c1cccc(F)c1C] 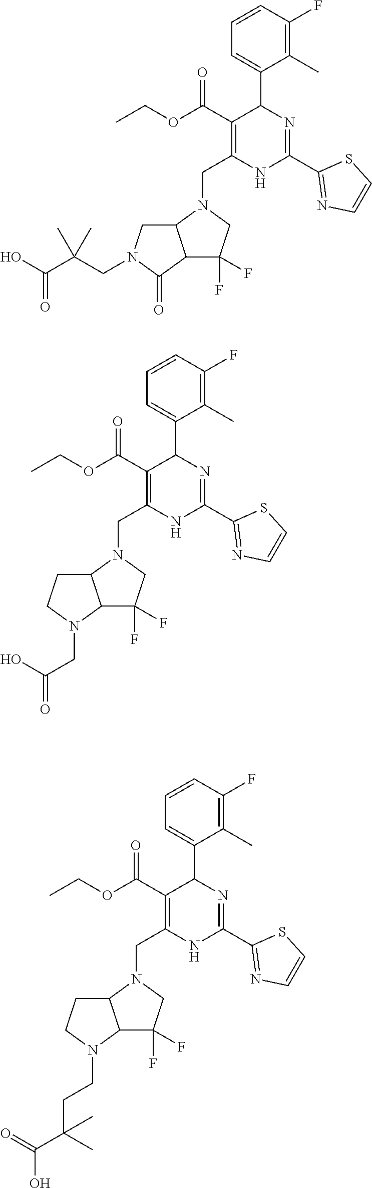 [SMILES: CCOC(=O)C1=C(CN2CC(F)(F)C3C(=O)N(CC(C)(C)C(=O)O)CC32)NC(c2nccs2)=NC1c1cccc(F)c1C ; CCOC(=O)C1=C(CN2CC(F)(F)C3C2CCN3CC(=O)O)NC(c2nccs2)=NC1c1cccc(F)c1C ; CCOC(=O)C1=C(CN2CC(F)(F)C3C2CCN3CCC(C)(C)C(=O)O)NC(c2nccs2)=NC1c1cccc(F)c1C] 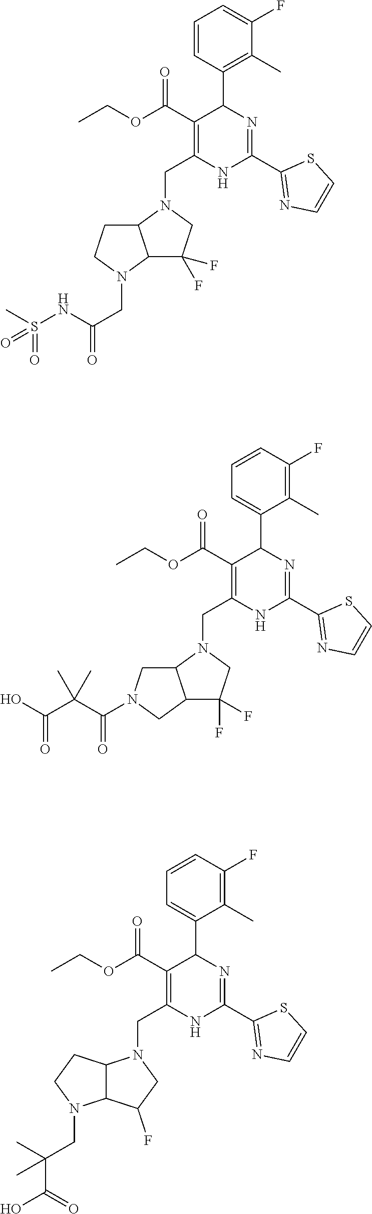 [SMILES: CCOC(=O)C1=C(CN2CC(F)(F)C3C2CCN3CC(=O)NS(C)(=O)=O)NC(c2nccs2)=NC1c1cccc(F)c1C ; CCOC(=O)C1=C(CN2CC(F)(F)C3CN(C(=O)C(C)(C)C(=O)O)CC32)NC(c2nccs2)=NC1c1cccc(F)c1C ; CCOC(=O)C1=C(CN2CC(F)C3C2CCN3CC(C)(C)C(=O)O)NC(c2nccs2)=NC1c1cccc(F)c1C] 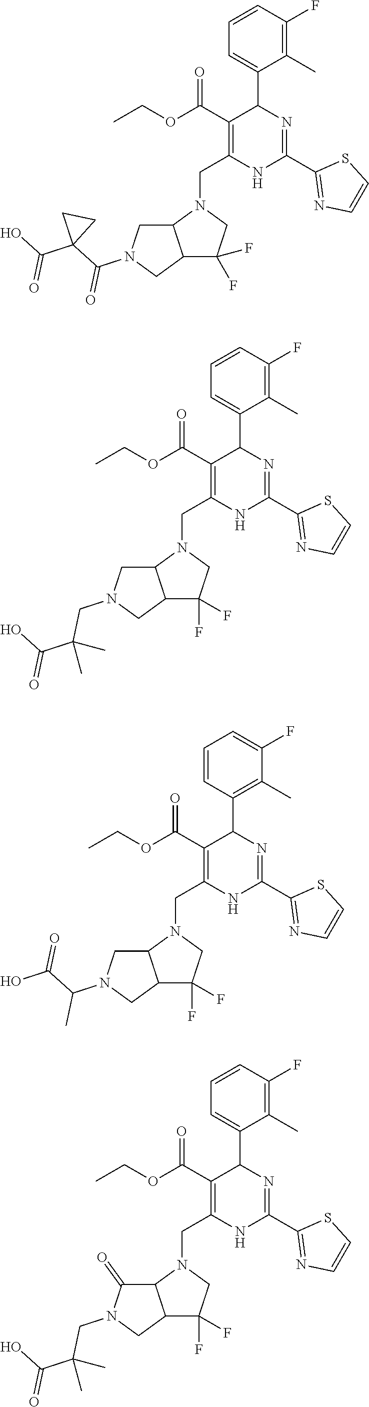 [SMILES: CCOC(=O)C1=C(CN2CC(F)(F)C3CN(C(=O)C4(C(=O)O)CC4)CC32)NC(c2nccs2)=NC1c1cccc(F)c1C ; CCOC(=O)C1=C(CN2CC(F)(F)C3CN(CC(C)(C)C(=O)O)CC32)NC(c2nccs2)=NC1c1cccc(F)c1C ; CCOC(=O)C1=C(CN2CC(F)(F)C3CN(C(C)C(=O)O)CC32)NC(c2nccs2)=NC1c1cccc(F)c1C ; CCOC(=O)C1=C(CN2CC(F)(F)C3CN(CC(C)(C)C(=O)O)C(=O)C32)NC(c2nccs2)=NC1c1cccc(F)c1C] 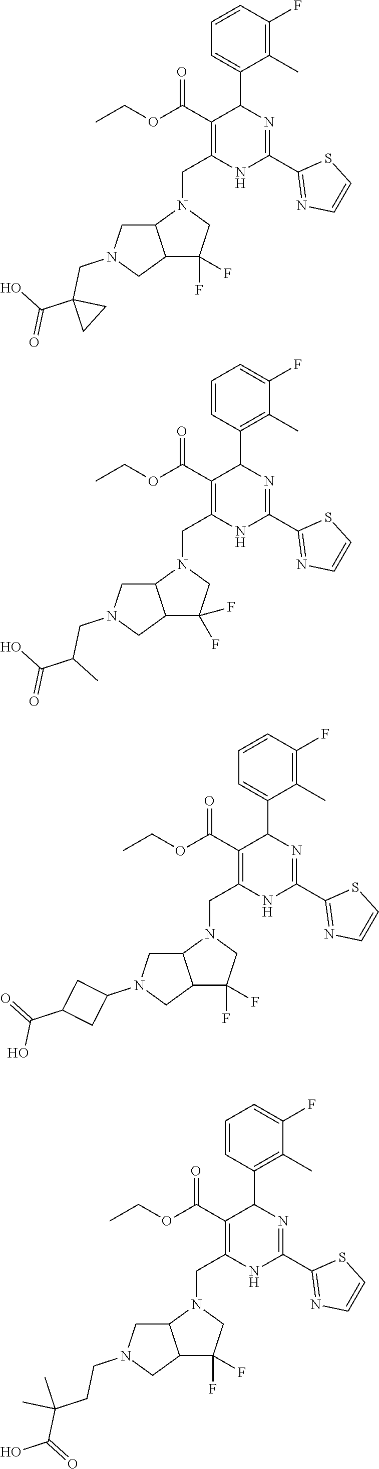 [SMILES: CCOC(=O)C1=C(CN2CC(F)(F)C3CN(CC4(C(=O)O)CC4)CC32)NC(c2nccs2)=NC1c1cccc(F)c1C ; CCOC(=O)C1=C(CN2CC(F)(F)C3CN(CC(C)C(=O)O)CC32)NC(c2nccs2)=NC1c1cccc(F)c1C ; CCOC(=O)C1=C(CN2CC(F)(F)C3CN(C4CC(C(=O)O)C4)CC32)NC(c2nccs2)=NC1c1cccc(F)c1C ; CCOC(=O)C1=C(CN2CC(F)(F)C3CN(CCC(C)(C)C(=O)O)CC32)NC(c2nccs2)=NC1c1cccc(F)c1C] 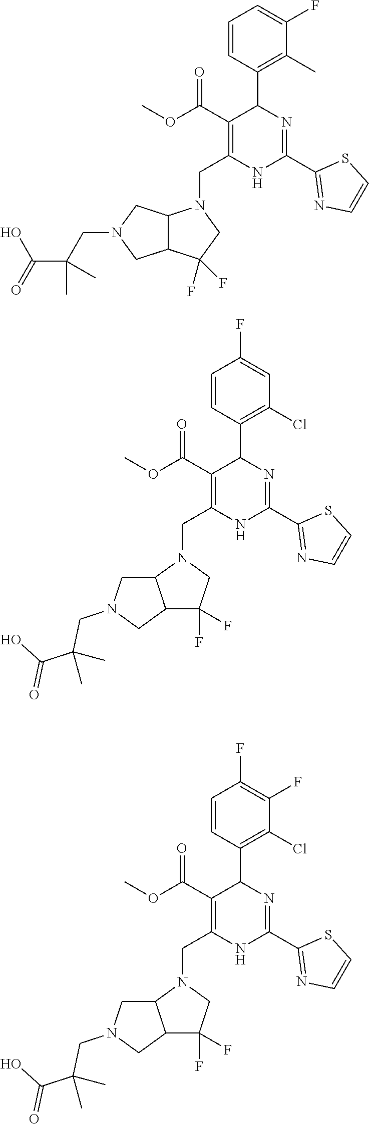 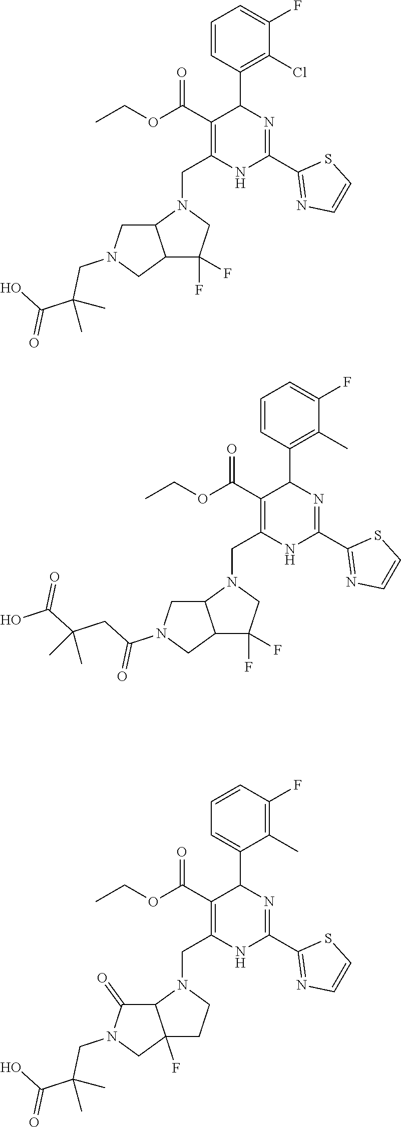 [SMILES: CCOC(=O)C1=C(CN2CC(F)(F)C3CN(CC(C)(C)C(=O)O)CC32)NC(c2nccs2)=NC1c1cccc(F)c1Cl ; CCOC(=O)C1=C(CN2CC(F)(F)C3CN(C(=O)CC(C)(C)C(=O)O)CC32)NC(c2nccs2)=NC1c1cccc(F)c1C ; CCOC(=O)C1=C(CN2CCC3(F)CN(CC(C)(C)C(=O)O)C(=O)C23)NC(c2nccs2)=NC1c1cccc(F)c1C] 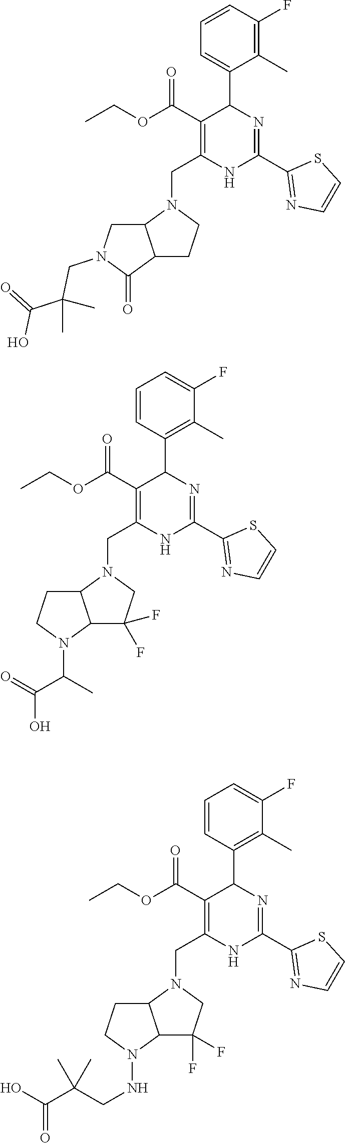 [SMILES: CCOC(=O)C1=C(CN2CCC3C(=O)N(CC(C)(C)C(=O)O)CC32)NC(c2nccs2)=NC1c1cccc(F)c1C ; CCOC(=O)C1=C(CN2CC(F)(F)C3C2CCN3C(C)C(=O)O)NC(c2nccs2)=NC1c1cccc(F)c1C ; CCOC(=O)C1=C(CN2CC(F)(F)C3C2CCN3NCC(C)(C)C(=O)O)NC(c2nccs2)=NC1c1cccc(F)c1C] 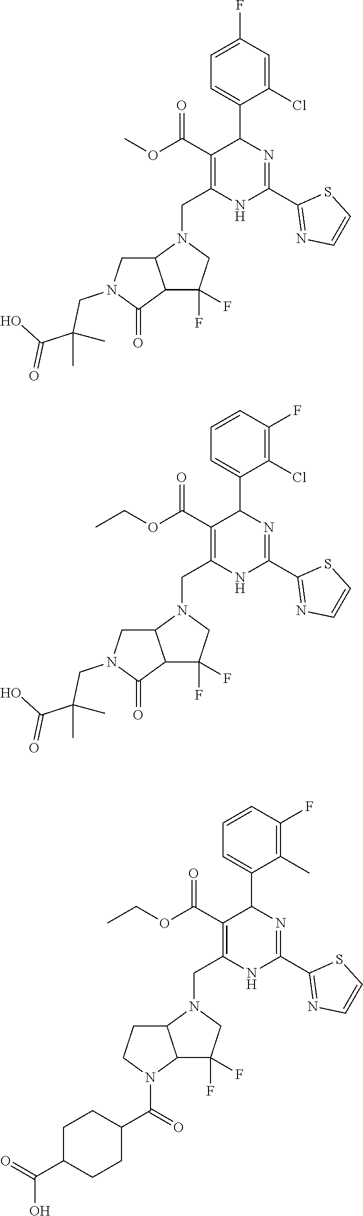 [SMILES: COC(=O)C1=C(CN2CC(F)(F)C3C(=O)N(CC(C)(C)C(=O)O)CC32)NC(c2nccs2)=NC1c1ccc(F)cc1Cl ; CCOC(=O)C1=C(CN2CC(F)(F)C3C(=O)N(CC(C)(C)C(=O)O)CC32)NC(c2nccs2)=NC1c1cccc(F)c1Cl ; CCOC(=O)C1=C(CN2CC(F)(F)C3C2CCN3C(=O)C2CCC(C(=O)O)CC2)NC(c2nccs2)=NC1c1cccc(F)c1C] 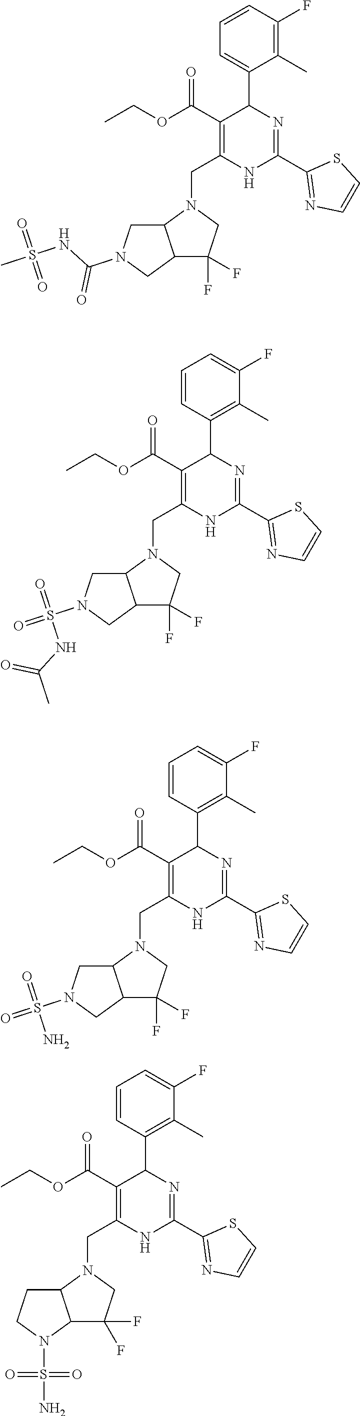 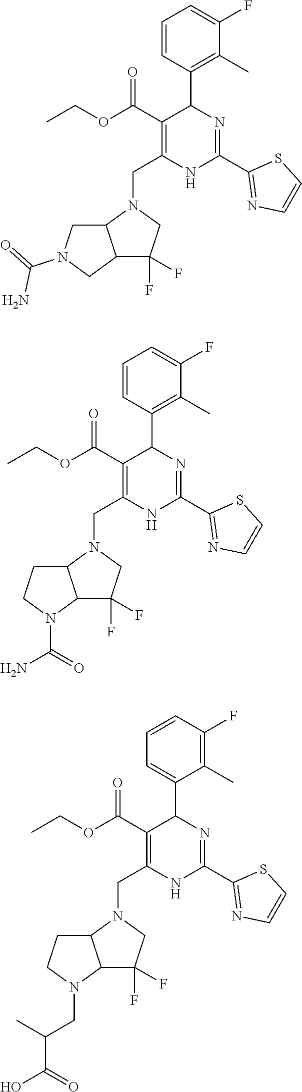 [SMILES: CCOC(=O)C1=C(CN2CC(F)(F)C3CN(C(N)=O)CC32)NC(c2nccs2)=NC1c1cccc(F)c1C ; CCOC(=O)C1=C(CN2CC(F)(F)C3C2CCN3C(N)=O)NC(c2nccs2)=NC1c1cccc(F)c1C ; CCOC(=O)C1=C(CN2CC(F)(F)C3C2CCN3CC(C)C(=O)O)NC(c2nccs2)=NC1c1cccc(F)c1C] 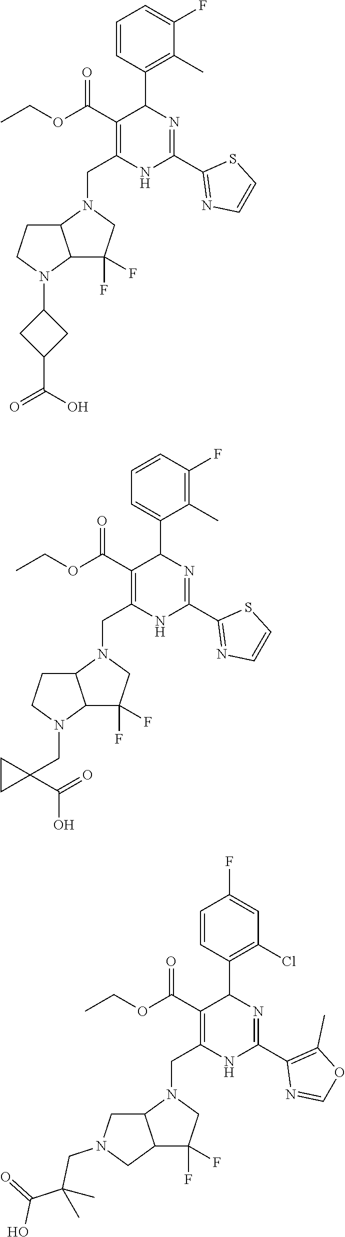 [SMILES: CCOC(=O)C1=C(CN2CC(F)(F)C3C2CCN3C2CC(C(=O)O)C2)NC(c2nccs2)=NC1c1cccc(F)c1C ; CCOC(=O)C1=C(CN2CC(F)(F)C3C2CCN3CC2(C(=O)O)CC2)NC(c2nccs2)=NC1c1cccc(F)c1C ; CCOC(=O)C1=C(CN2CC(F)(F)C3CN(CC(C)(C)C(=O)O)CC32)NC(c2ncoc2C)=NC1c1ccc(F)cc1Cl] 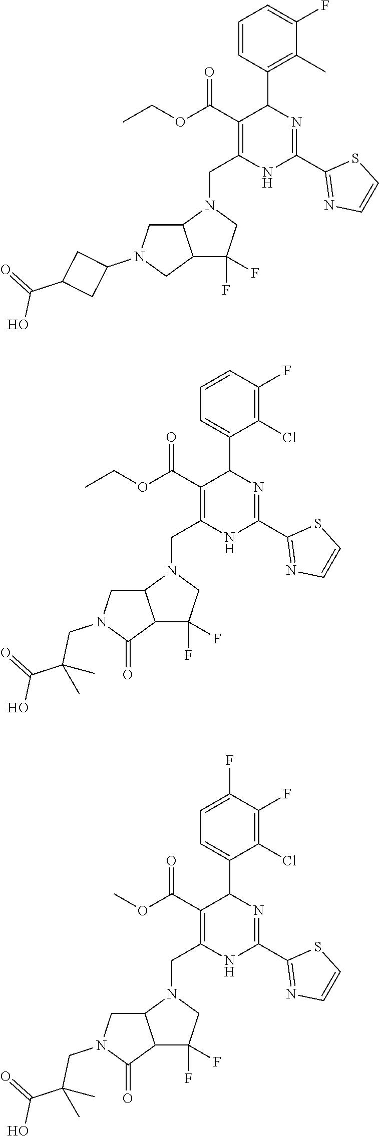 [SMILES: CCOC(=O)C1=C(CN2CC(F)(F)C3CN(C4CC(C(=O)O)C4)CC32)NC(c2nccs2)=NC1c1cccc(F)c1C ; CCOC(=O)C1=C(CN2CC(F)(F)C3C(=O)N(CC(C)(C)C(=O)O)CC32)NC(c2nccs2)=NC1c1cccc(F)c1Cl ; COC(=O)C1=C(CN2CC(F)(F)C3C(=O)N(CC(C)(C)C(=O)O)CC32)NC(c2nccs2)=NC1c1ccc(F)c(F)c1Cl] 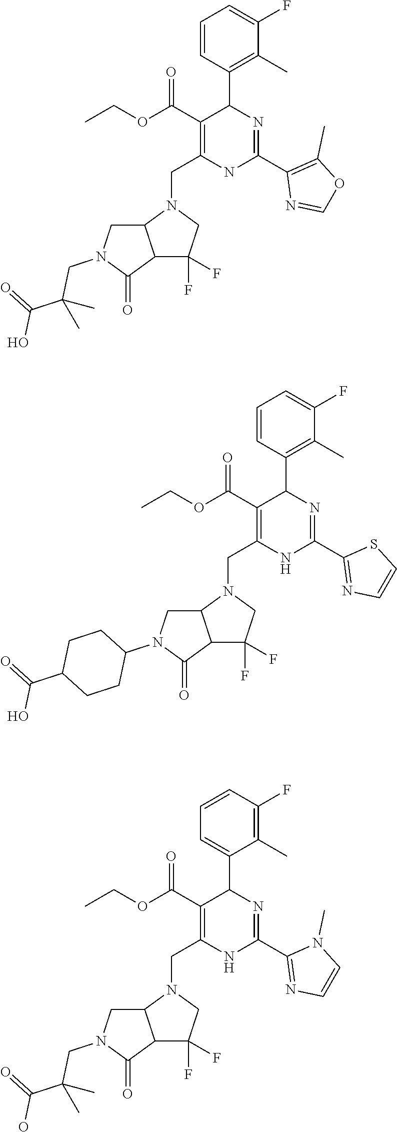 [SMILES: CCOC(=O)C1=C(CN2CC(F)(F)C3C(=O)N(CC(C)(C)C(=O)O)CC32)NC(c2ncoc2C)=NC1c1cccc(F)c1C ; CCOC(=O)C1=C(CN2CC(F)(F)C3C(=O)N(C4CCC(C(=O)O)CC4)CC32)NC(c2nccs2)=NC1c1cccc(F)c1C ; CCOC(=O)C1=C(CN2CC(F)(F)C3C(=O)N(CC(C)(C)C(=O)O)CC32)NC(c2nccn2C)=NC1c1cccc(F)c1C] 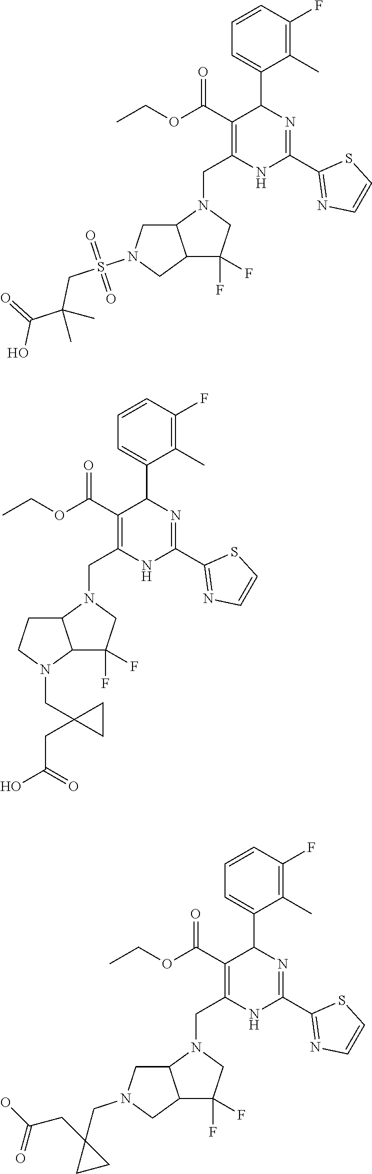 [SMILES: CCOC(=O)C1=C(CN2CC(F)(F)C3CN(S(=O)(=O)CC(C)(C)C(=O)O)CC32)NC(c2nccs2)=NC1c1cccc(F)c1C ; CCOC(=O)C1=C(CN2CC(F)(F)C3C2CCN3CC2(CC(=O)O)CC2)NC(c2nccs2)=NC1c1cccc(F)c1C ; CCOC(=O)C1=C(CN2CC(F)(F)C3CN(CC4(CC(=O)O)CC4)CC32)NC(c2nccs2)=NC1c1cccc(F)c1C] 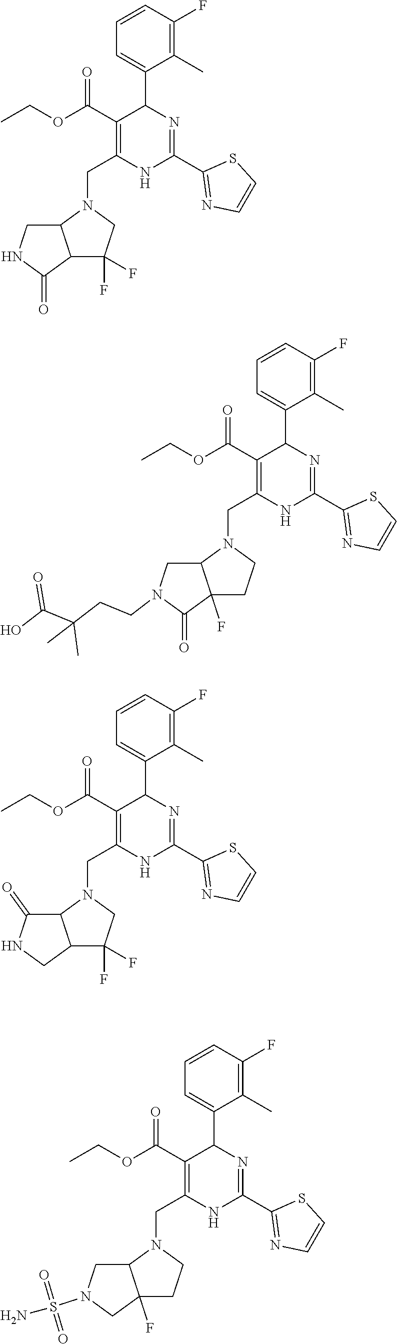 [SMILES: CCOC(=O)C1=C(CN2CC(F)(F)C3C(=O)NCC32)NC(c2nccs2)=NC1c1cccc(F)c1C ; CCOC(=O)C1=C(CN2CCC3(F)C(=O)N(CCC(C)(C)C(=O)O)CC23)NC(c2nccs2)=NC1c1cccc(F)c1C ; CCOC(=O)C1=C(CN2CC(F)(F)C3CNC(=O)C32)NC(c2nccs2)=NC1c1cccc(F)c1C ; CCOC(=O)C1=C(CN2CCC3(F)CN(S(N)(=O)=O)CC23)NC(c2nccs2)=NC1c1cccc(F)c1C] 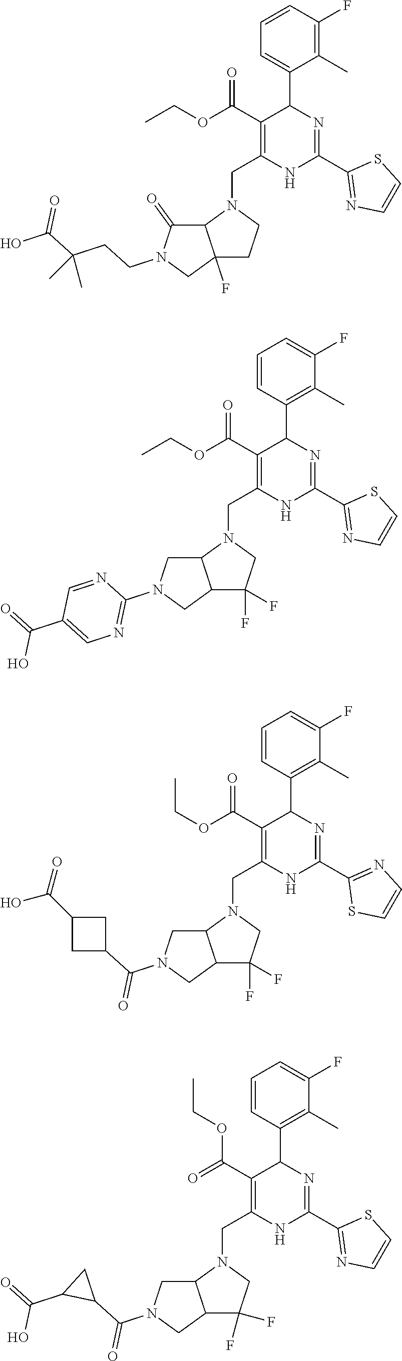 [SMILES: CCOC(=O)C1=C(CN2CCC3(F)CN(CCC(C)(C)C(=O)O)C(=O)C23)NC(c2nccs2)=NC1c1cccc(F)c1C ; CCOC(=O)C1=C(CN2CC(F)(F)C3CN(c4ncc(C(=O)O)cn4)CC32)NC(c2nccs2)=NC1c1cccc(F)c1C ; CCOC(=O)C1=C(CN2CC(F)(F)C3CN(C(=O)C4CC(C(=O)O)C4)CC32)NC(c2nccs2)=NC1c1cccc(F)c1C ; CCOC(=O)C1=C(CN2CC(F)(F)C3CN(C(=O)C4CC4C(=O)O)CC32)NC(c2nccs2)=NC1c1cccc(F)c1C] 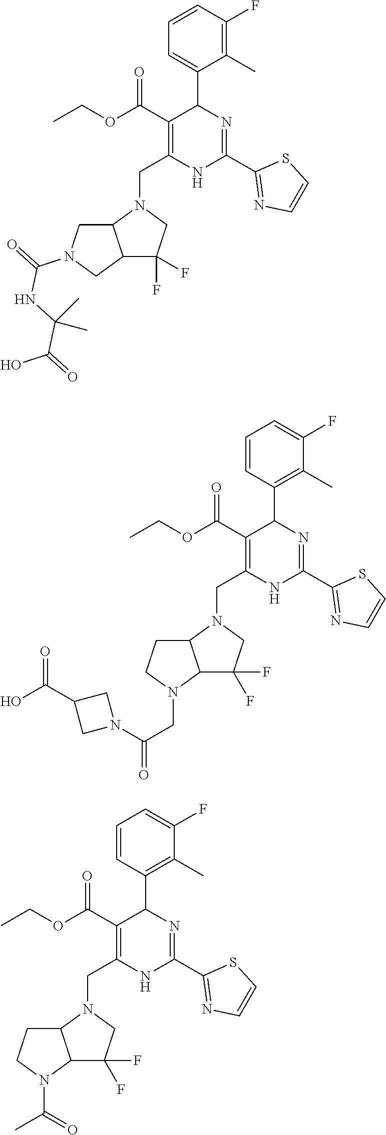 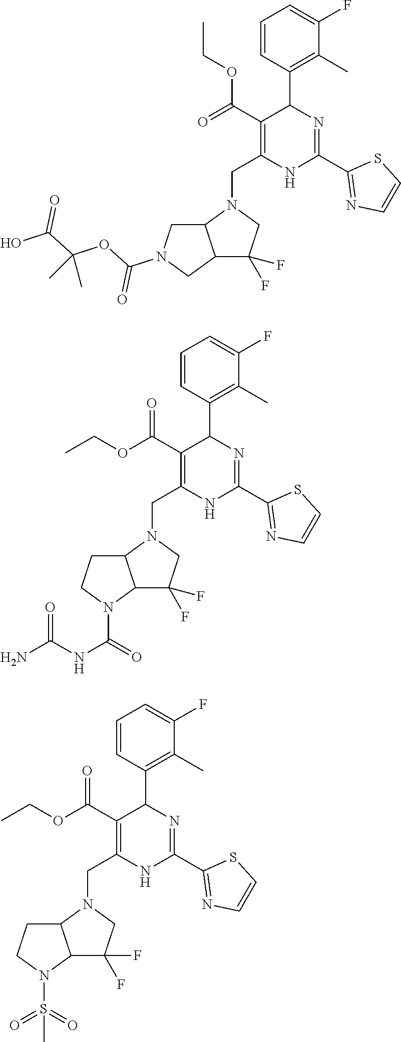 [SMILES: CCOC(=O)C1=C(CN2CC(F)(F)C3CN(C(=O)OC(C)(C)C(=O)O)CC32)NC(c2nccs2)=NC1c1cccc(F)c1C ; CCOC(=O)C1=C(CN2CC(F)(F)C3C2CCN3C(=O)NC(N)=O)NC(c2nccs2)=NC1c1cccc(F)c1C ; CCOC(=O)C1=C(CN2CC(F)(F)C3C2CCN3S(C)(=O)=O)NC(c2nccs2)=NC1c1cccc(F)c1C] 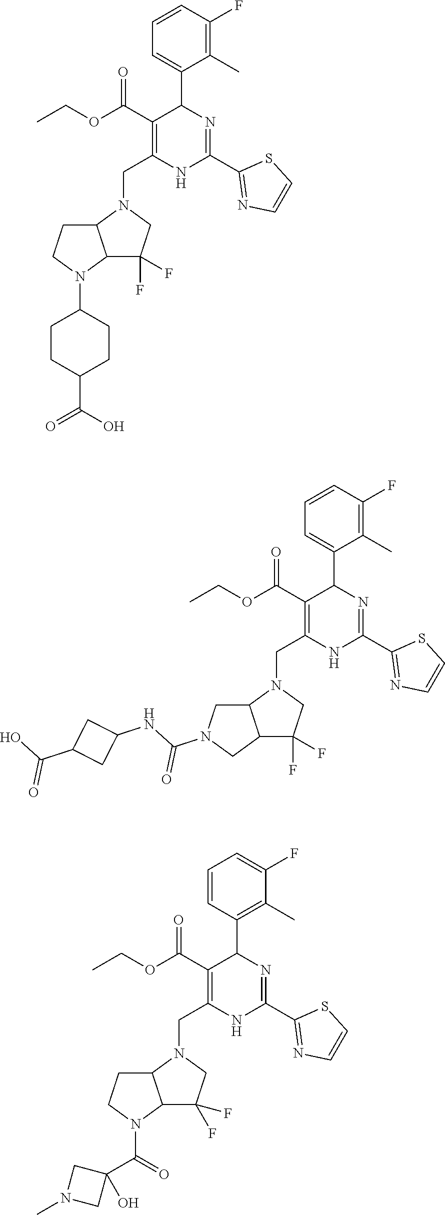 [SMILES: CCOC(=O)C1=C(CN2CC(F)(F)C3C2CCN3C2CCC(C(=O)O)CC2)NC(c2nccs2)=NC1c1cccc(F)c1C ; CCOC(=O)C1=C(CN2CC(F)(F)C3CN(C(=O)NC4CC(C(=O)O)C4)CC32)NC(c2nccs2)=NC1c1cccc(F)c1C ; CCOC(=O)C1=C(CN2CC(F)(F)C3C2CCN3C(=O)C2(O)CN(C)C2)NC(c2nccs2)=NC1c1cccc(F)c1C] 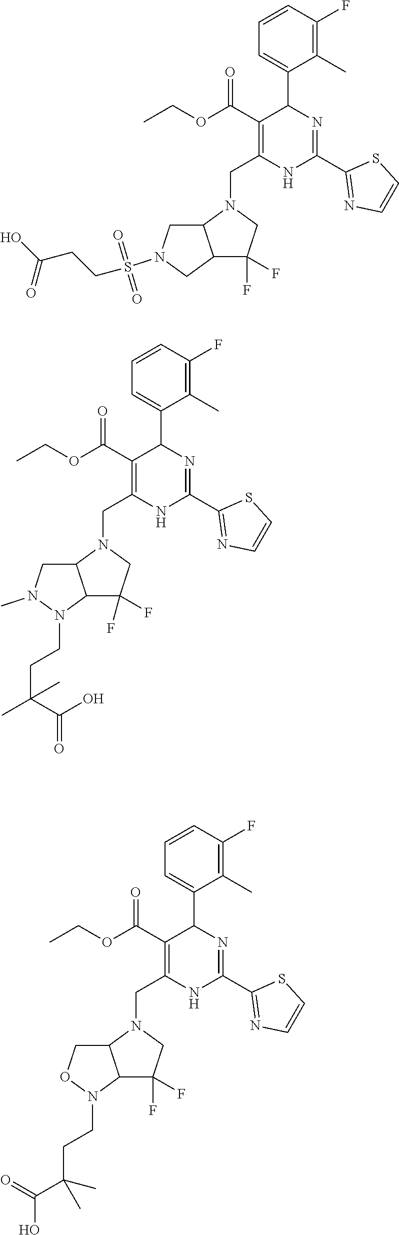 [SMILES: CCOC(=O)C1=C(CN2CC(F)(F)C3CN(S(=O)(=O)CCC(=O)O)CC32)NC(c2nccs2)=NC1c1cccc(F)c1C ; CCOC(=O)C1=C(CN2CC(F)(F)C3C2CN(C)N3CCC(C)(C)C(=O)O)NC(c2nccs2)=NC1c1cccc(F)c1C ; CCOC(=O)C1=C(CN2CC(F)(F)C3C2CON3CCC(C)(C)C(=O)O)NC(c2nccs2)=NC1c1cccc(F)c1C] 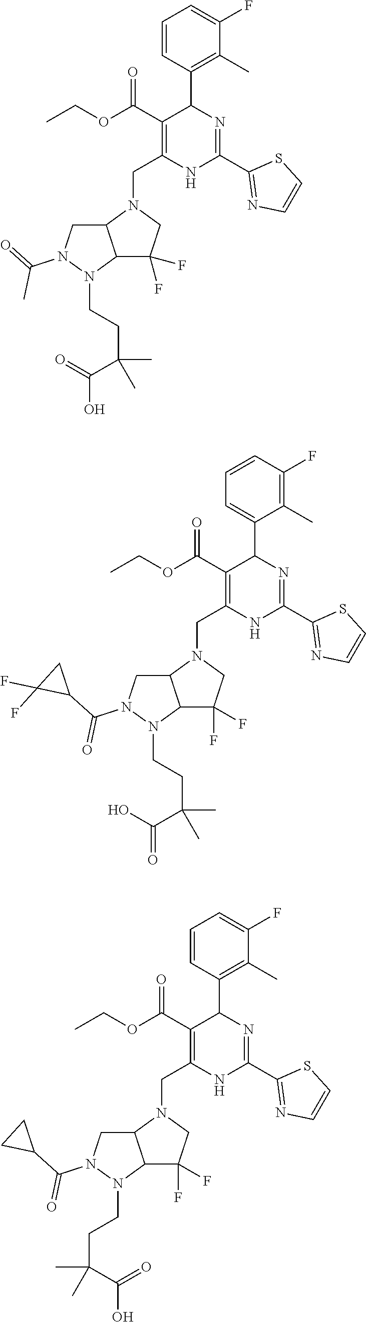 [SMILES: CCOC(=O)C1=C(CN2CC(F)(F)C3C2CN(C(C)=O)N3CCC(C)(C)C(=O)O)NC(c2nccs2)=NC1c1cccc(F)c1C ; CCOC(=O)C1=C(CN2CC(F)(F)C3C2CN(C(=O)C2CC2(F)F)N3CCC(C)(C)C(=O)O)NC(c2nccs2)=NC1c1cccc(F)c1C ; CCOC(=O)C1=C(CN2CC(F)(F)C3C2CN(C(=O)C2CC2)N3CCC(C)(C)C(=O)O)NC(c2nccs2)=NC1c1cccc(F)c1C] 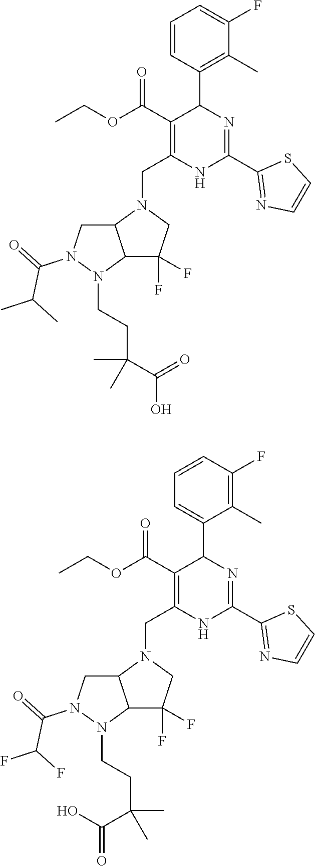 [SMILES: CCOC(=O)C1=C(CN2CC(F)(F)C3C2CN(C(=O)C(C)C)N3CCC(C)(C)C(=O)O)NC(c2nccs2)=NC1c1cccc(F)c1C ; CCOC(=O)C1=C(CN2CC(F)(F)C3C2CN(C(=O)C(F)F)N3CCC(C)(C)C(=O)O)NC(c2nccs2)=NC1c1cccc(F)c1C] 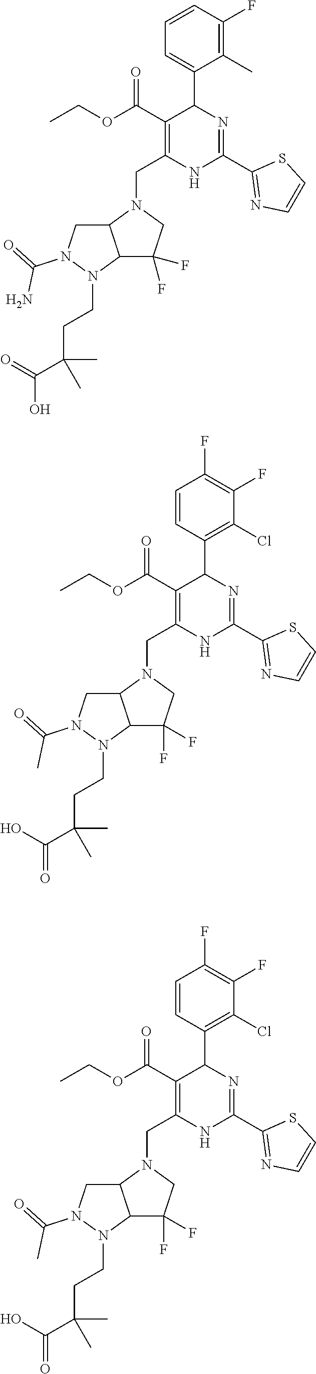 [SMILES: CCOC(=O)C1=C(CN2CC(F)(F)C3C2CN(C(N)=O)N3CCC(C)(C)C(=O)O)NC(c2nccs2)=NC1c1cccc(F)c1C ; CCOC(=O)C1=C(CN2CC(F)(F)C3C2CN(C(C)=O)N3CCC(C)(C)C(=O)O)NC(c2nccs2)=NC1c1ccc(F)c(F)c1Cl ; CCOC(=O)C1=C(CN2CC(F)(F)C3C2CN(C(C)=O)N3CCC(C)(C)C(=O)O)NC(c2nccs2)=NC1c1ccc(F)c(F)c1Cl] 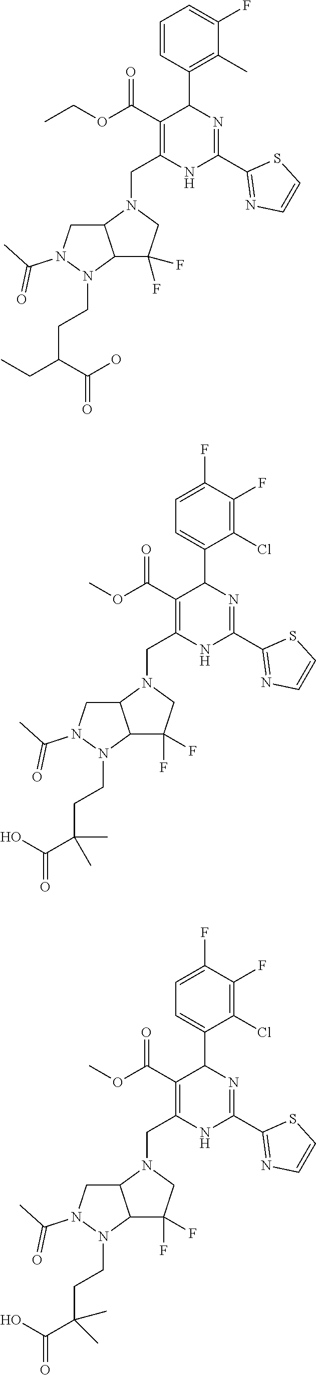 [SMILES: CCOC(=O)C1=C(CN2CC(F)(F)C3C2CN(C(C)=O)N3CCC(CC)C(=O)O)NC(c2nccs2)=NC1c1cccc(F)c1C ; COC(=O)C1=C(CN2CC(F)(F)C3C2CN(C(C)=O)N3CCC(C)(C)C(=O)O)NC(c2nccs2)=NC1c1ccc(F)c(F)c1Cl ; COC(=O)C1=C(CN2CC(F)(F)C3C2CN(C(C)=O)N3CCC(C)(C)C(=O)O)NC(c2nccs2)=NC1c1ccc(F)c(F)c1Cl] 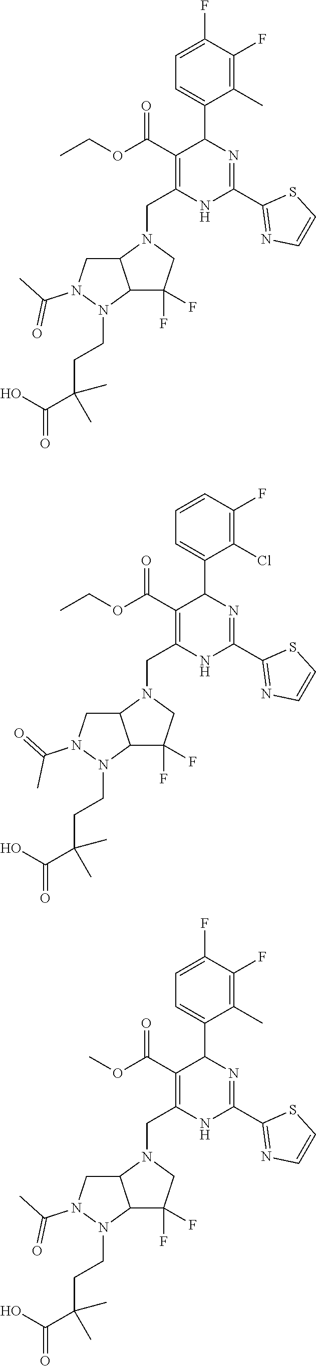 [SMILES: CCOC(=O)C1=C(CN2CC(F)(F)C3C2CN(C(C)=O)N3CCC(C)(C)C(=O)O)NC(c2nccs2)=NC1c1ccc(F)c(F)c1C ; CCOC(=O)C1=C(CN2CC(F)(F)C3C2CN(C(C)=O)N3CCC(C)(C)C(=O)O)NC(c2nccs2)=NC1c1cccc(F)c1Cl ; COC(=O)C1=C(CN2CC(F)(F)C3C2CN(C(C)=O)N3CCC(C)(C)C(=O)O)NC(c2nccs2)=NC1c1ccc(F)c(F)c1C] 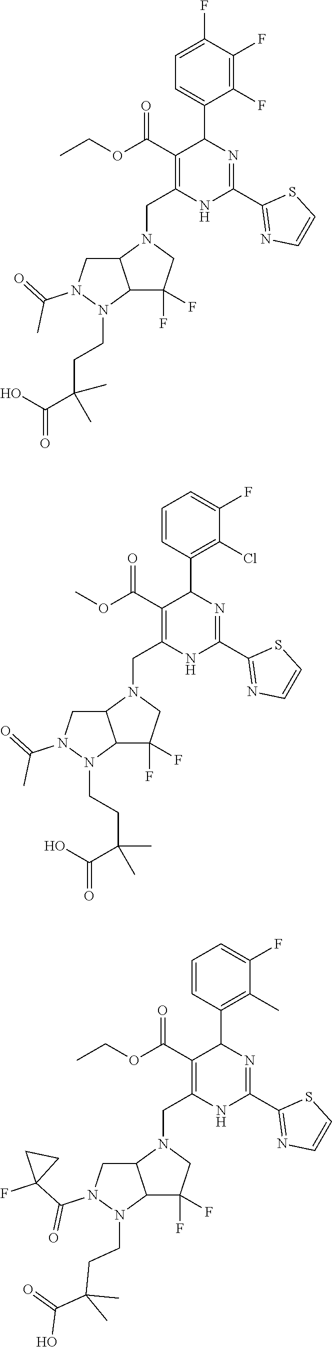 [SMILES: CCOC(=O)C1=C(CN2CC(F)(F)C3C2CN(C(C)=O)N3CCC(C)(C)C(=O)O)NC(c2nccs2)=NC1c1ccc(F)c(F)c1F ; COC(=O)C1=C(CN2CC(F)(F)C3C2CN(C(C)=O)N3CCC(C)(C)C(=O)O)NC(c2nccs2)=NC1c1cccc(F)c1Cl ; CCOC(=O)C1=C(CN2CC(F)(F)C3C2CN(C(=O)C2(F)CC2)N3CCC(C)(C)C(=O)O)NC(c2nccs2)=NC1c1cccc(F)c1C] 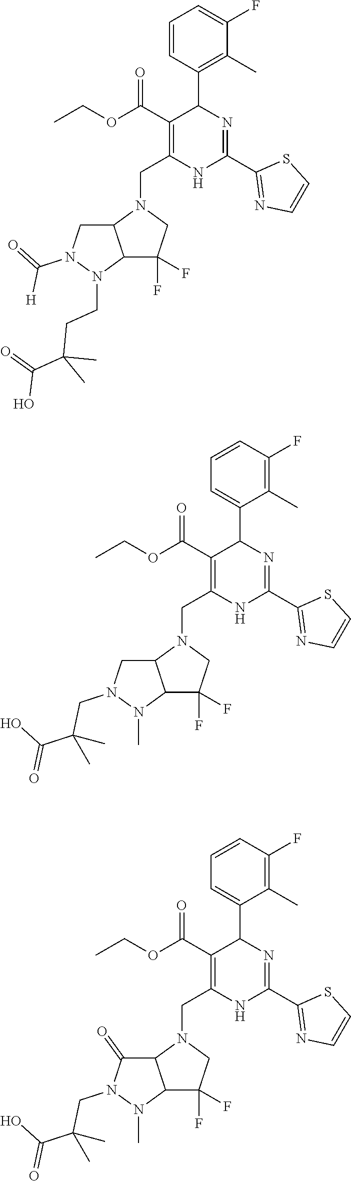 [SMILES: CCOC(=O)C1=C(CN2CC(F)(F)C3C2CN(C=O)N3CCC(C)(C)C(=O)O)NC(c2nccs2)=NC1c1cccc(F)c1C ; CCOC(=O)C1=C(CN2CC(F)(F)C3C2CN(CC(C)(C)C(=O)O)N3C)NC(c2nccs2)=NC1c1cccc(F)c1C ; CCOC(=O)C1=C(CN2CC(F)(F)C3C2C(=O)N(CC(C)(C)C(=O)O)N3C)NC(c2nccs2)=NC1c1cccc(F)c1C] 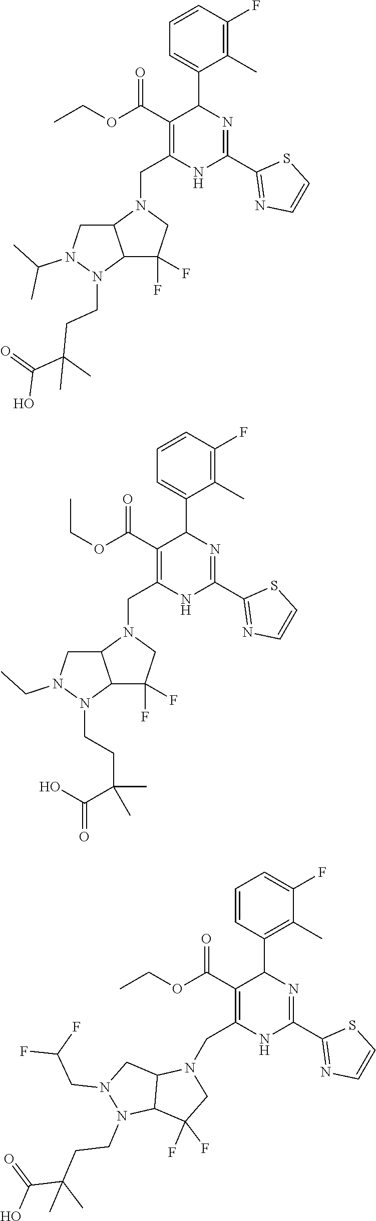 [SMILES: CCOC(=O)C1=C(CN2CC(F)(F)C3C2CN(C(C)C)N3CCC(C)(C)C(=O)O)NC(c2nccs2)=NC1c1cccc(F)c1C ; CCOC(=O)C1=C(CN2CC(F)(F)C3C2CN(CC)N3CCC(C)(C)C(=O)O)NC(c2nccs2)=NC1c1cccc(F)c1C ; CCOC(=O)C1=C(CN2CC(F)(F)C3C2CN(CC(F)F)N3CCC(C)(C)C(=O)O)NC(c2nccs2)=NC1c1cccc(F)c1C] 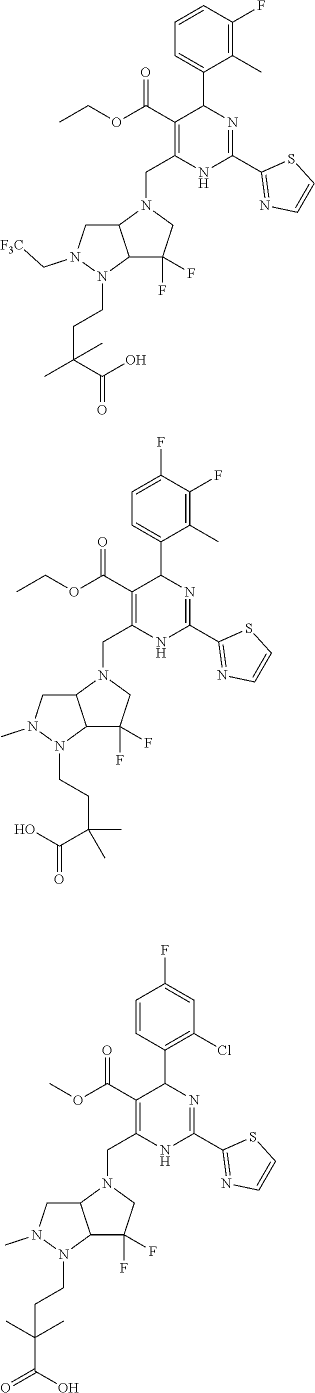 [SMILES: CCOC(=O)C1=C(CN2CC(F)(F)C3C2CN(CC(F)(F)F)N3CCC(C)(C)C(=O)O)NC(c2nccs2)=NC1c1cccc(F)c1C ; CCOC(=O)C1=C(CN2CC(F)(F)C3C2CN(C)N3CCC(C)(C)C(=O)O)NC(c2nccs2)=NC1c1ccc(F)c(F)c1C ; COC(=O)C1=C(CN2CC(F)(F)C3C2CN(C)N3CCC(C)(C)C(=O)O)NC(c2nccs2)=NC1c1ccc(F)cc1Cl] 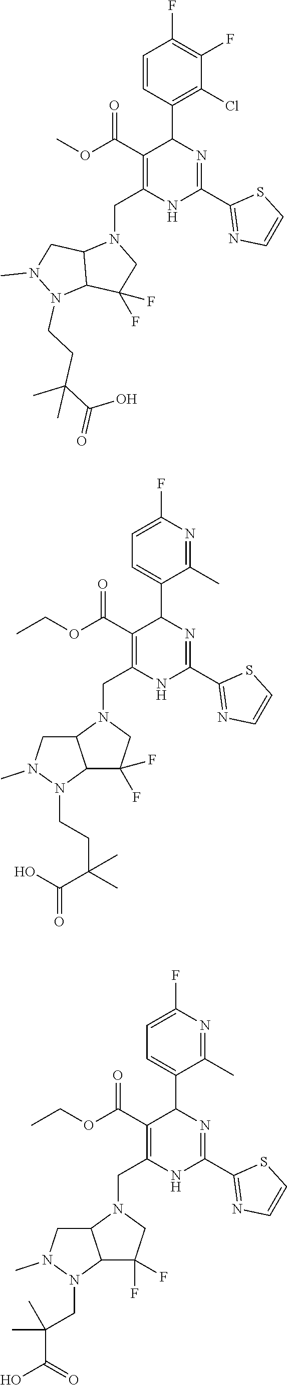 [SMILES: COC(=O)C1=C(CN2CC(F)(F)C3C2CN(C)N3CCC(C)(C)C(=O)O)NC(c2nccs2)=NC1c1ccc(F)c(F)c1Cl ; CCOC(=O)C1=C(CN2CC(F)(F)C3C2CN(C)N3CCC(C)(C)C(=O)O)NC(c2nccs2)=NC1c1ccc(F)nc1C ; CCOC(=O)C1=C(CN2CC(F)(F)C3C2CN(C)N3CC(C)(C)C(=O)O)NC(c2nccs2)=NC1c1ccc(F)nc1C] 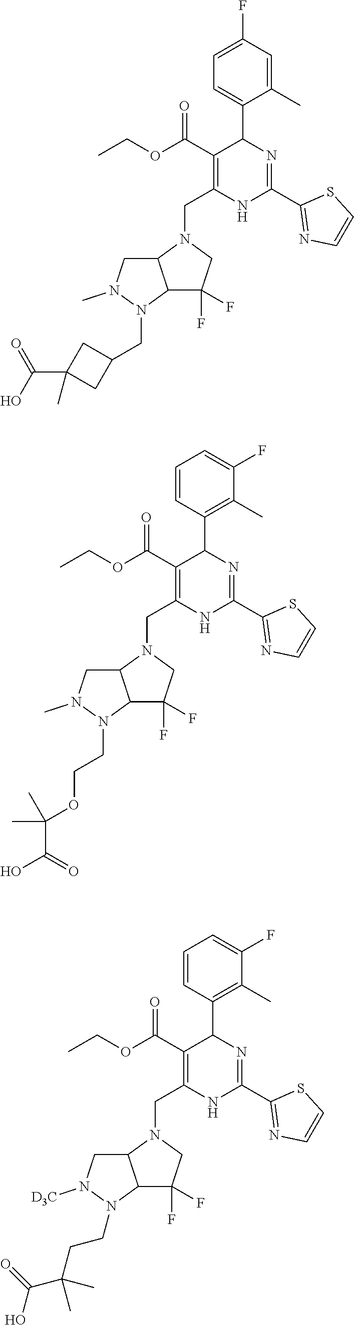 [SMILES: CCOC(=O)C1=C(CN2CC(F)(F)C3C2CN(C)N3CC2CC(C)(C(=O)O)C2)NC(c2nccs2)=NC1c1ccc(F)cc1C ; CCOC(=O)C1=C(CN2CC(F)(F)C3C2CN(C)N3CCOC(C)(C)C(=O)O)NC(c2nccs2)=NC1c1cccc(F)c1C ; [2H]C([2H])([2H])N1CC2C(N1CCC(C)(C)C(=O)O)C(F)(F)CN2CC1=C(C(=O)OCC)C(c2cccc(F)c2C)N=C(c2nccs2)N1] 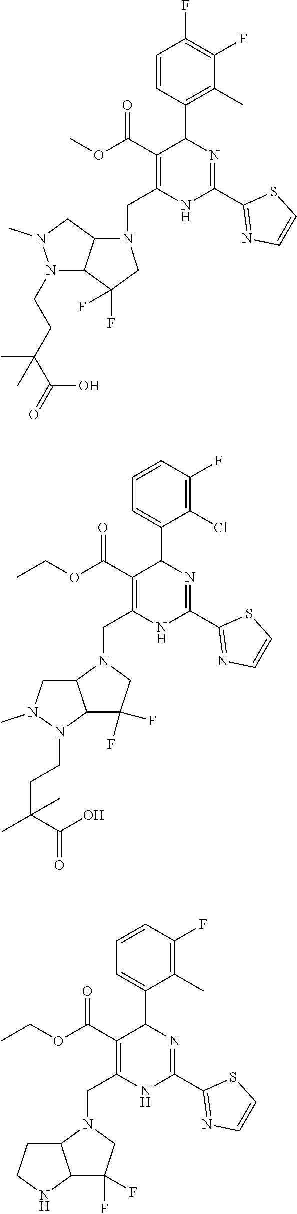 [SMILES: COC(=O)C1=C(CN2CC(F)(F)C3C2CN(C)N3CCC(C)(C)C(=O)O)NC(c2nccs2)=NC1c1ccc(F)c(F)c1C ; CCOC(=O)C1=C(CN2CC(F)(F)C3C2CN(C)N3CCC(C)(C)C(=O)O)NC(c2nccs2)=NC1c1cccc(F)c1Cl ; CCOC(=O)C1=C(CN2CC(F)(F)C3NCCC32)NC(c2nccs2)=NC1c1cccc(F)c1C] 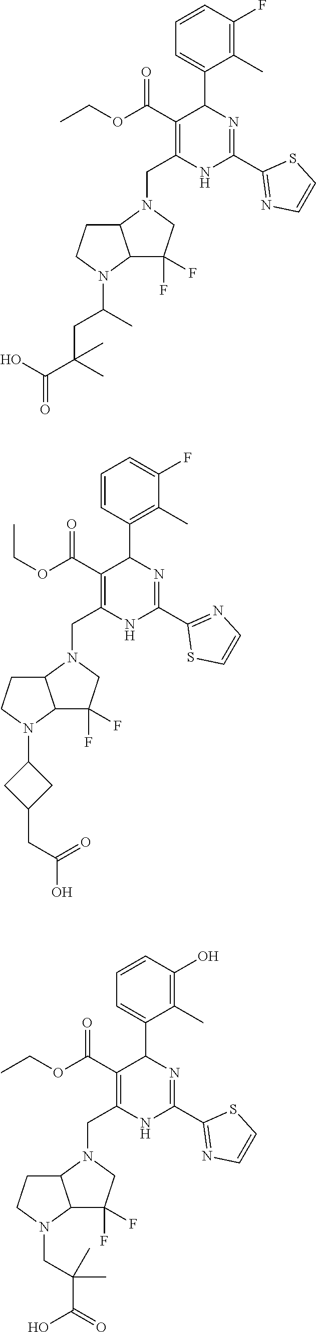 [SMILES: CCOC(=O)C1=C(CN2CC(F)(F)C3C2CCN3C(C)CC(C)(C)C(=O)O)NC(c2nccs2)=NC1c1cccc(F)c1C ; CCOC(=O)C1=C(CN2CC(F)(F)C3C2CCN3C2CC(CC(=O)O)C2)NC(c2nccs2)=NC1c1cccc(F)c1C ; CCOC(=O)C1=C(CN2CC(F)(F)C3C2CCN3CC(C)(C)C(=O)O)NC(c2nccs2)=NC1c1cccc(O)c1C] 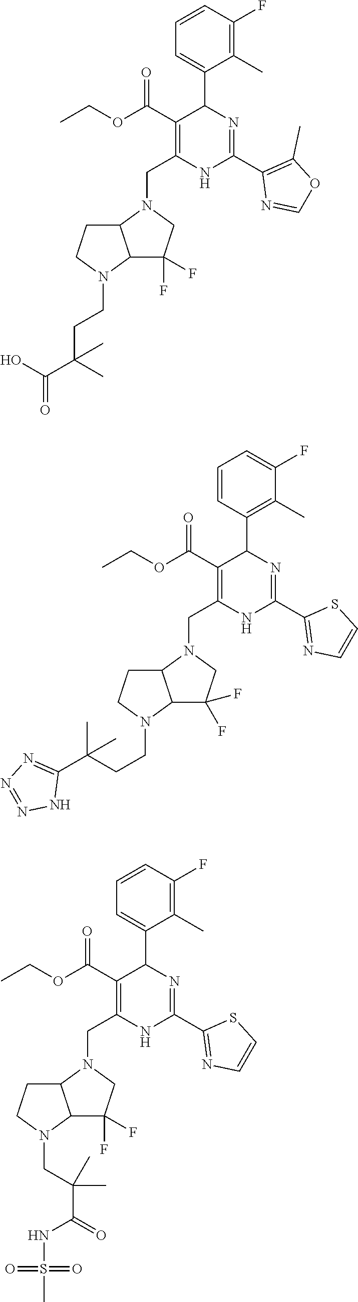 [SMILES: CCOC(=O)C1=C(CN2CC(F)(F)C3C2CCN3CCC(C)(C)C(=O)O)NC(c2ncoc2C)=NC1c1cccc(F)c1C ; CCOC(=O)C1=C(CN2CC(F)(F)C3C2CCN3CCC(C)(C)c2nnn[nH]2)NC(c2nccs2)=NC1c1cccc(F)c1C ; CCOC(=O)C1=C(CN2CC(F)(F)C3C2CCN3CC(C)(C)C(=O)NS(C)(=O)=O)NC(c2nccs2)=NC1c1cccc(F)c1C] 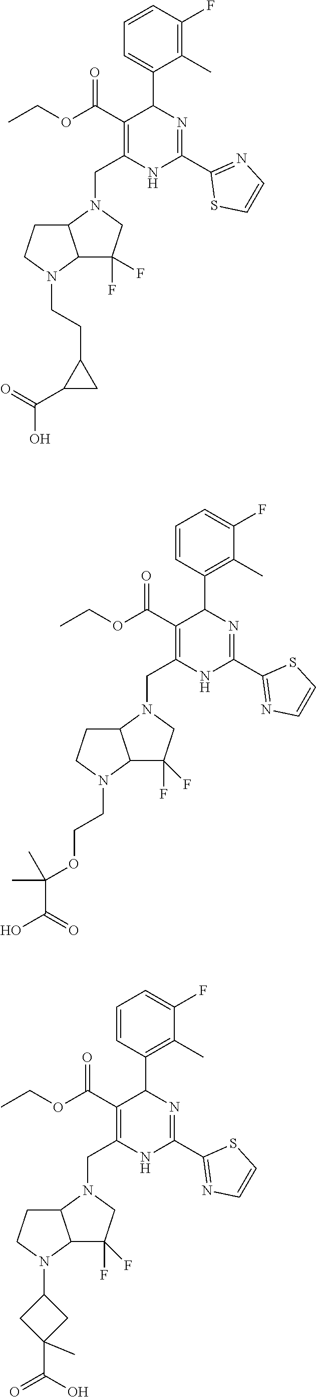 [SMILES: CCOC(=O)C1=C(CN2CC(F)(F)C3C2CCN3CCC2CC2C(=O)O)NC(c2nccs2)=NC1c1cccc(F)c1C ; CCOC(=O)C1=C(CN2CC(F)(F)C3C2CCN3CCOC(C)(C)C(=O)O)NC(c2nccs2)=NC1c1cccc(F)c1C ; CCOC(=O)C1=C(CN2CC(F)(F)C3C2CCN3C2CC(C)(C(=O)O)C2)NC(c2nccs2)=NC1c1cccc(F)c1C] 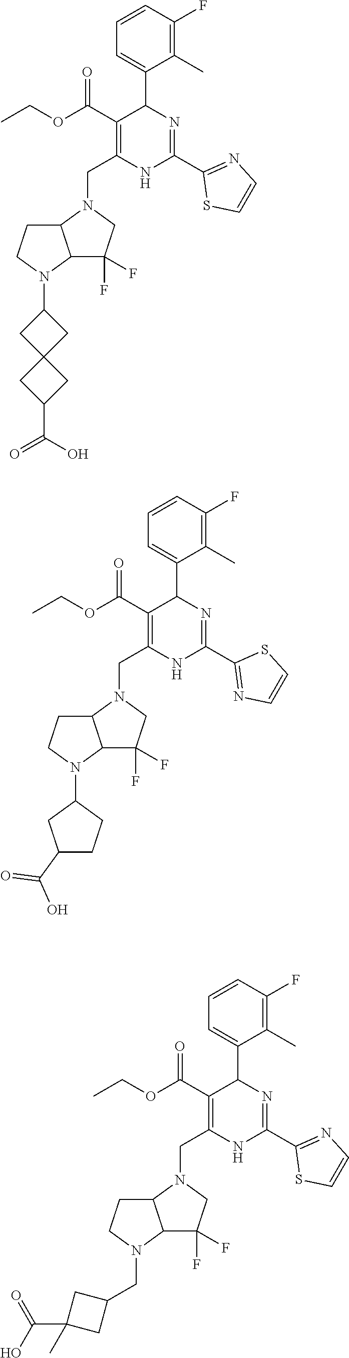 [SMILES: CCOC(=O)C1=C(CN2CC(F)(F)C3C2CCN3C2CC3(CC(C(=O)O)C3)C2)NC(c2nccs2)=NC1c1cccc(F)c1C ; CCOC(=O)C1=C(CN2CC(F)(F)C3C2CCN3C2CCC(C(=O)O)C2)NC(c2nccs2)=NC1c1cccc(F)c1C ; CCOC(=O)C1=C(CN2CC(F)(F)C3C2CCN3CC2CC(C)(C(=O)O)C2)NC(c2nccs2)=NC1c1cccc(F)c1C] 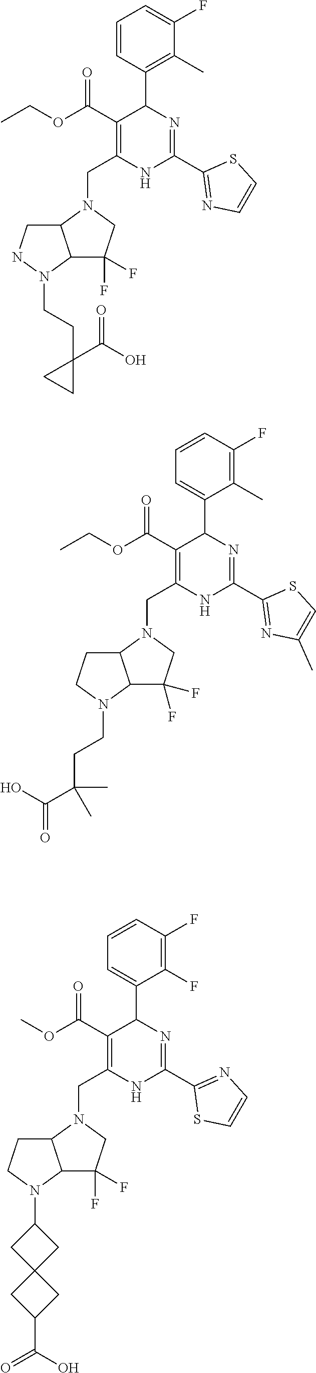 [SMILES: CCOC(=O)C1=C(CN2CC(F)(F)C3C2CNN3CCC2(C(=O)O)CC2)NC(c2nccs2)=NC1c1cccc(F)c1C ; CCOC(=O)C1=C(CN2CC(F)(F)C3C2CCN3CCC(C)(C)C(=O)O)NC(c2nc(C)cs2)=NC1c1cccc(F)c1C ; COC(=O)C1=C(CN2CC(F)(F)C3C2CCN3C2CC3(CC(C(=O)O)C3)C2)NC(c2nccs2)=NC1c1cccc(F)c1F] 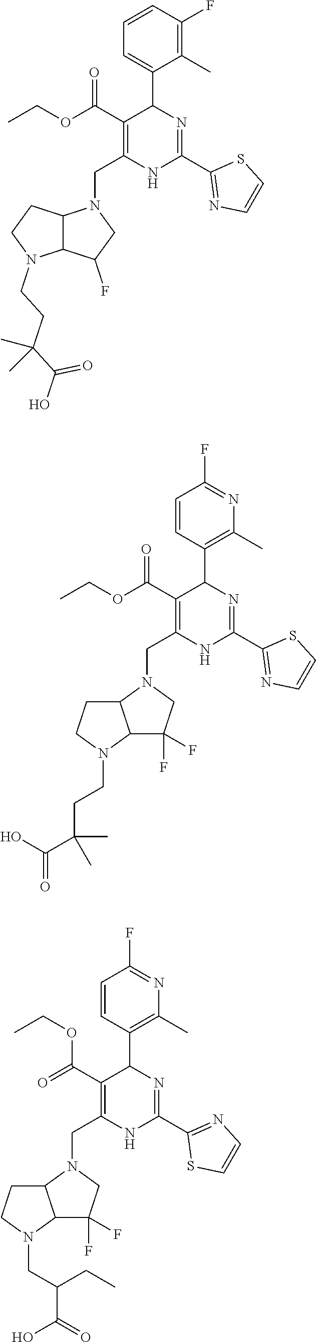 [SMILES: CCOC(=O)C1=C(CN2CC(F)C3C2CCN3CCC(C)(C)C(=O)O)NC(c2nccs2)=NC1c1cccc(F)c1C ; CCOC(=O)C1=C(CN2CC(F)(F)C3C2CCN3CCC(C)(C)C(=O)O)NC(c2nccs2)=NC1c1ccc(F)nc1C ; CCOC(=O)C1=C(CN2CC(F)(F)C3C2CCN3CC(CC)C(=O)O)NC(c2nccs2)=NC1c1ccc(F)nc1C] 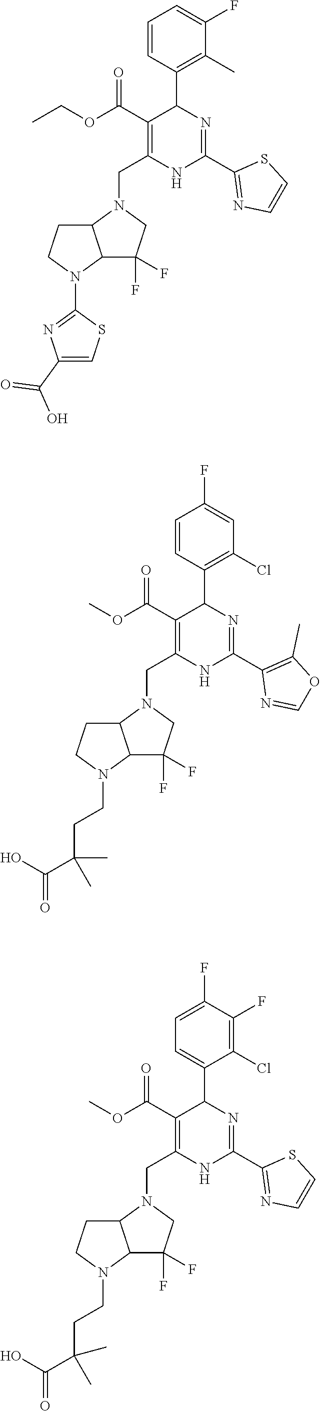 [SMILES: CCOC(=O)C1=C(CN2CC(F)(F)C3C2CCN3c2nc(C(=O)O)cs2)NC(c2nccs2)=NC1c1cccc(F)c1C ; COC(=O)C1=C(CN2CC(F)(F)C3C2CCN3CCC(C)(C)C(=O)O)NC(c2ncoc2C)=NC1c1ccc(F)cc1Cl ; COC(=O)C1=C(CN2CC(F)(F)C3C2CCN3CCC(C)(C)C(=O)O)NC(c2nccs2)=NC1c1ccc(F)c(F)c1Cl] 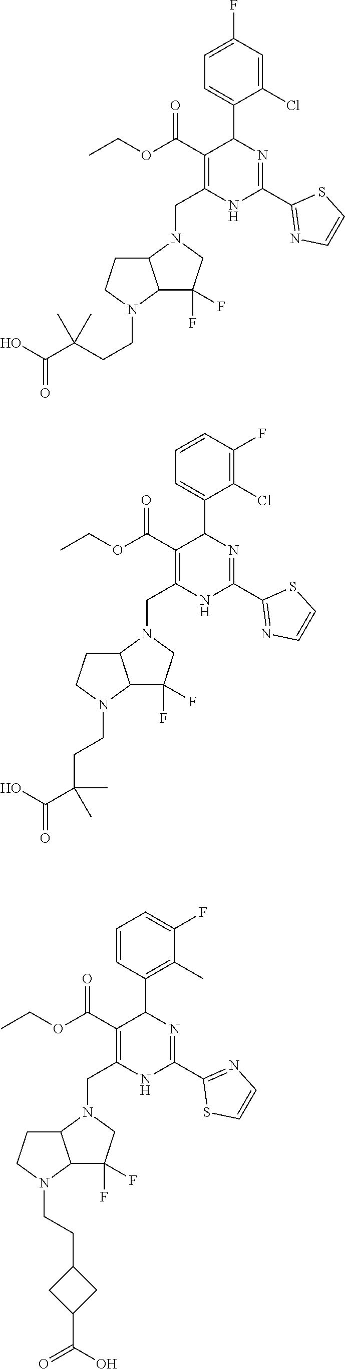 [SMILES: CCOC(=O)C1=C(CN2CC(F)(F)C3C2CCN3CCC(C)(C)C(=O)O)NC(c2nccs2)=NC1c1ccc(F)cc1Cl ; CCOC(=O)C1=C(CN2CC(F)(F)C3C2CCN3CCC(C)(C)C(=O)O)NC(c2nccs2)=NC1c1cccc(F)c1Cl ; CCOC(=O)C1=C(CN2CC(F)(F)C3C2CCN3CCC2CC(C(=O)O)C2)NC(c2nccs2)=NC1c1cccc(F)c1C] 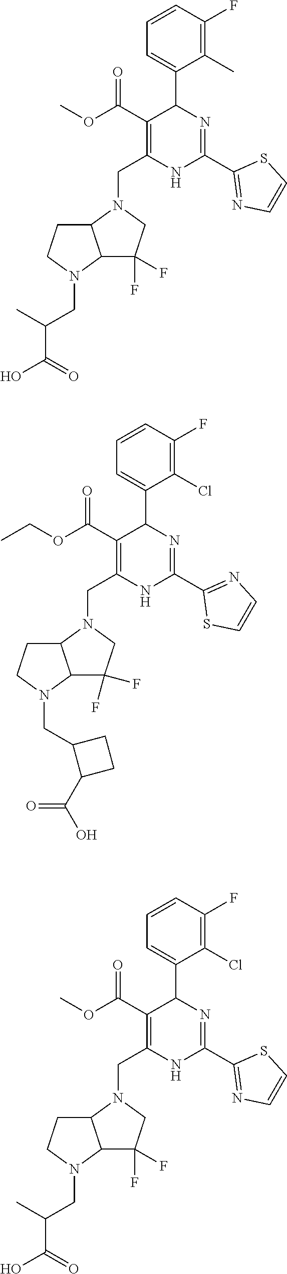 [SMILES: COC(=O)C1=C(CN2CC(F)(F)C3C2CCN3CC(C)C(=O)O)NC(c2nccs2)=NC1c1cccc(F)c1C ; CCOC(=O)C1=C(CN2CC(F)(F)C3C2CCN3CC2CCC2C(=O)O)NC(c2nccs2)=NC1c1cccc(F)c1Cl ; COC(=O)C1=C(CN2CC(F)(F)C3C2CCN3CC(C)C(=O)O)NC(c2nccs2)=NC1c1cccc(F)c1Cl] 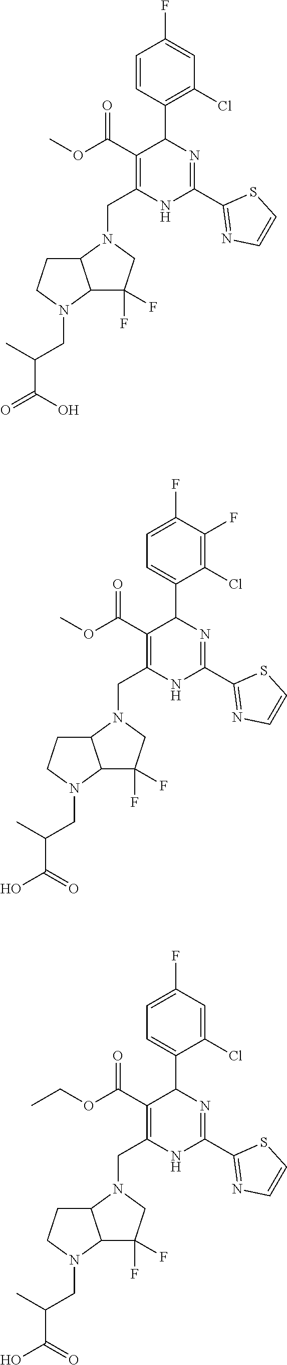 [SMILES: COC(=O)C1=C(CN2CC(F)(F)C3C2CCN3CC(C)C(=O)O)NC(c2nccs2)=NC1c1ccc(F)cc1Cl ; COC(=O)C1=C(CN2CC(F)(F)C3C2CCN3CC(C)C(=O)O)NC(c2nccs2)=NC1c1ccc(F)c(F)c1Cl ; CCOC(=O)C1=C(CN2CC(F)(F)C3C2CCN3CC(C)C(=O)O)NC(c2nccs2)=NC1c1ccc(F)cc1Cl] 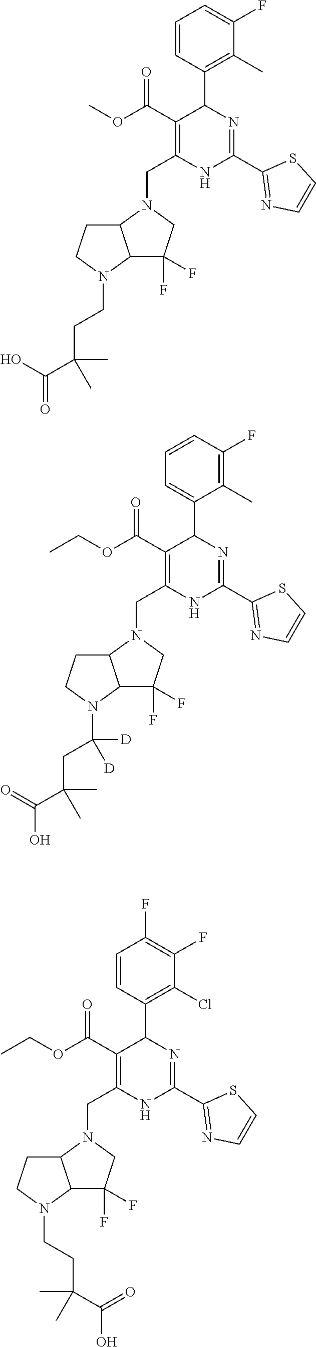 [SMILES: COC(=O)C1=C(CN2CC(F)(F)C3C2CCN3CCC(C)(C)C(=O)O)NC(c2nccs2)=NC1c1cccc(F)c1C ; [2H]C([2H])(CC(C)(C)C(=O)O)N1CCC2C1C(F)(F)CN2CC1=C(C(=O)OCC)C(c2cccc(F)c2C)N=C(c2nccs2)N1 ; CCOC(=O)C1=C(CN2CC(F)(F)C3C2CCN3CCC(C)(C)C(=O)O)NC(c2nccs2)=NC1c1ccc(F)c(F)c1Cl] 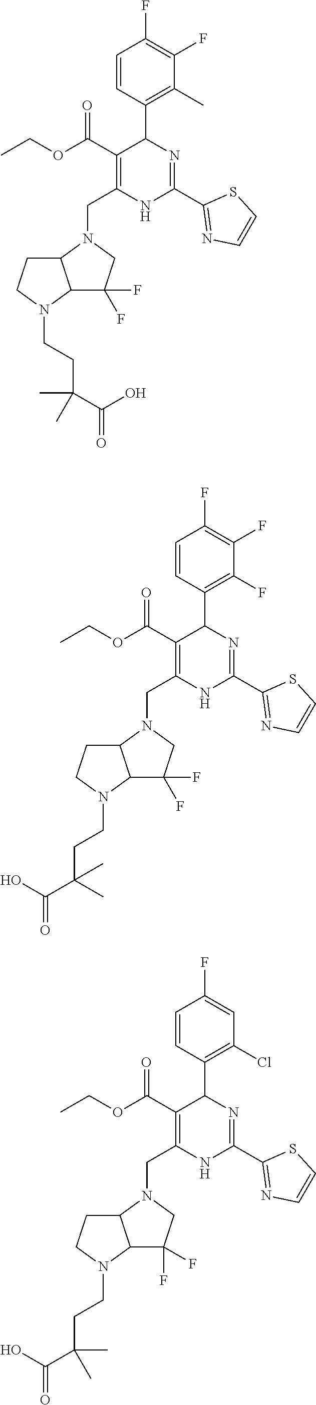 [SMILES: CCOC(=O)C1=C(CN2CC(F)(F)C3C2CCN3CCC(C)(C)C(=O)O)NC(c2nccs2)=NC1c1ccc(F)c(F)c1C ; CCOC(=O)C1=C(CN2CC(F)(F)C3C2CCN3CCC(C)(C)C(=O)O)NC(c2nccs2)=NC1c1ccc(F)c(F)c1F ; CCOC(=O)C1=C(CN2CC(F)(F)C3C2CCN3CCC(C)(C)C(=O)O)NC(c2nccs2)=NC1c1ccc(F)cc1Cl] 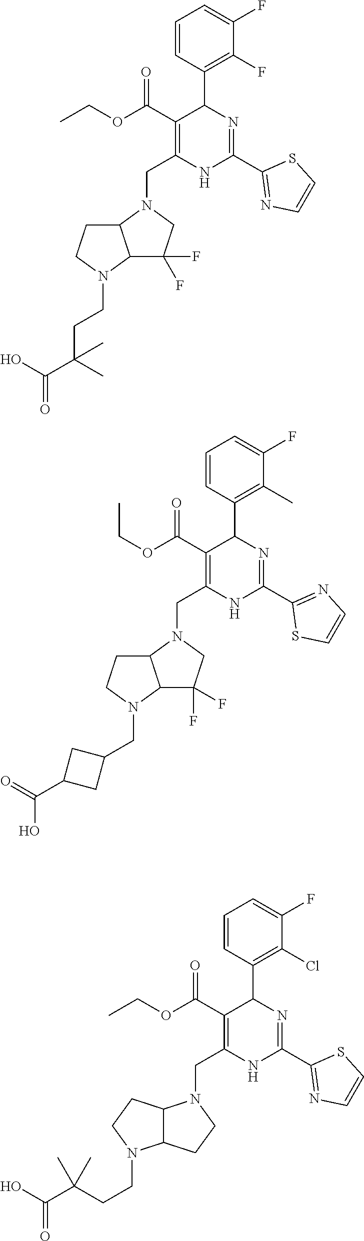 [SMILES: CCOC(=O)C1=C(CN2CC(F)(F)C3C2CCN3CCC(C)(C)C(=O)O)NC(c2nccs2)=NC1c1cccc(F)c1F ; CCOC(=O)C1=C(CN2CC(F)(F)C3C2CCN3CC2CC(C(=O)O)C2)NC(c2nccs2)=NC1c1cccc(F)c1C ; CCOC(=O)C1=C(CN2CCC3C2CCN3CCC(C)(C)C(=O)O)NC(c2nccs2)=NC1c1cccc(F)c1Cl] 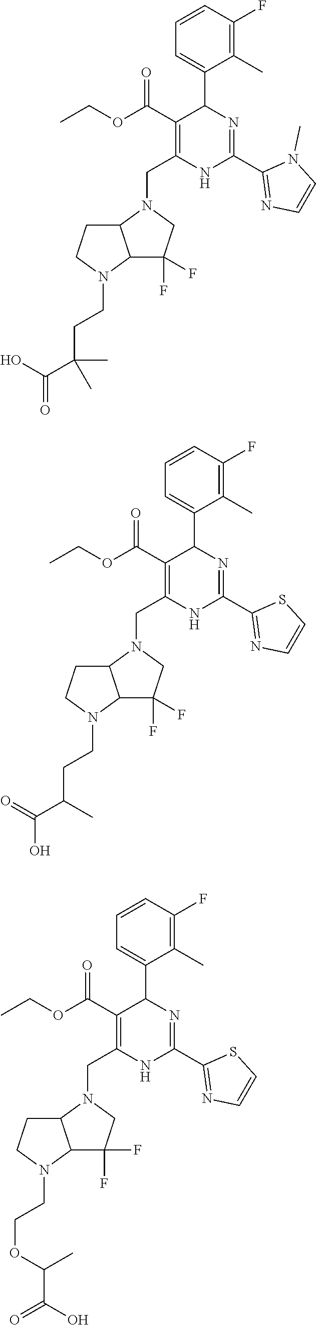 [SMILES: CCOC(=O)C1=C(CN2CC(F)(F)C3C2CCN3CCC(C)(C)C(=O)O)NC(c2nccn2C)=NC1c1cccc(F)c1C ; CCOC(=O)C1=C(CN2CC(F)(F)C3C2CCN3CCC(C)C(=O)O)NC(c2nccs2)=NC1c1cccc(F)c1C ; CCOC(=O)C1=C(CN2CC(F)(F)C3C2CCN3CCOC(C)C(=O)O)NC(c2nccs2)=NC1c1cccc(F)c1C] 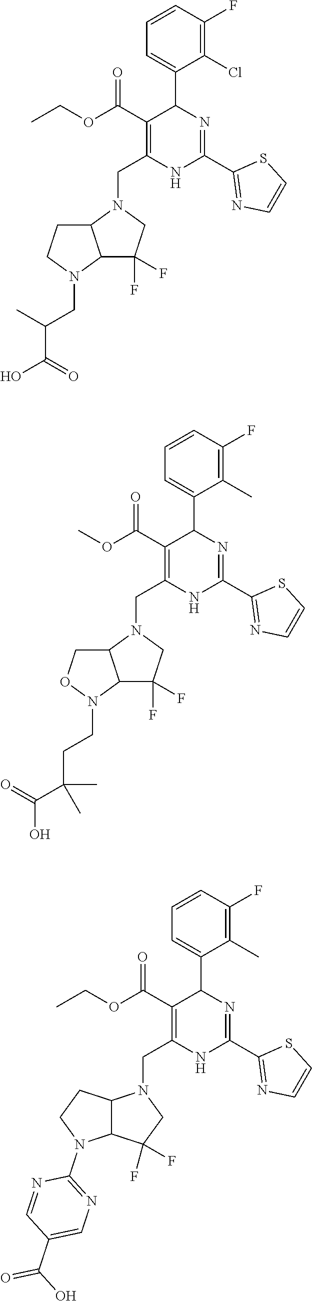 [SMILES: CCOC(=O)C1=C(CN2CC(F)(F)C3C2CCN3CC(C)C(=O)O)NC(c2nccs2)=NC1c1cccc(F)c1Cl ; COC(=O)C1=C(CN2CC(F)(F)C3C2CON3CCC(C)(C)C(=O)O)NC(c2nccs2)=NC1c1cccc(F)c1C ; CCOC(=O)C1=C(CN2CC(F)(F)C3C2CCN3c2ncc(C(=O)O)cn2)NC(c2nccs2)=NC1c1cccc(F)c1C] 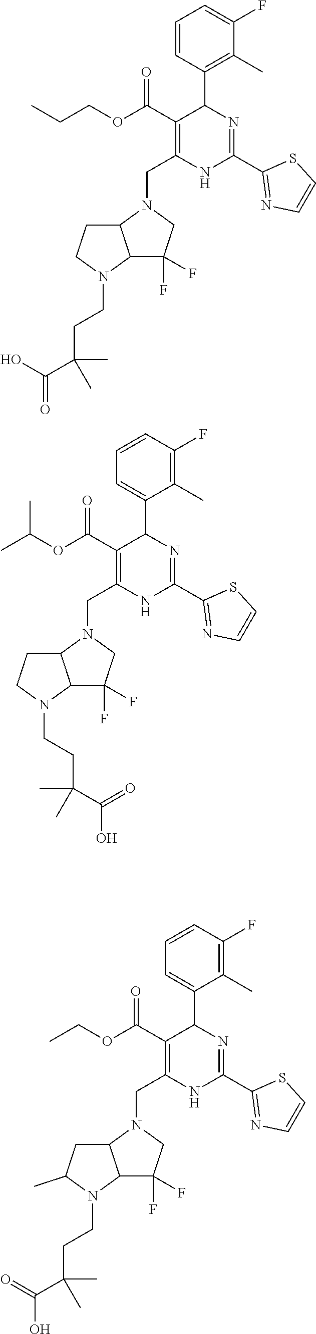 [SMILES: CCCOC(=O)C1=C(CN2CC(F)(F)C3C2CCN3CCC(C)(C)C(=O)O)NC(c2nccs2)=NC1c1cccc(F)c1C ; Cc1c(F)cccc1C1N=C(c2nccs2)NC(CN2CC(F)(F)C3C2CCN3CCC(C)(C)C(=O)O)=C1C(=O)OC(C)C ; CCOC(=O)C1=C(CN2CC(F)(F)C3C2CC(C)N3CCC(C)(C)C(=O)O)NC(c2nccs2)=NC1c1cccc(F)c1C] 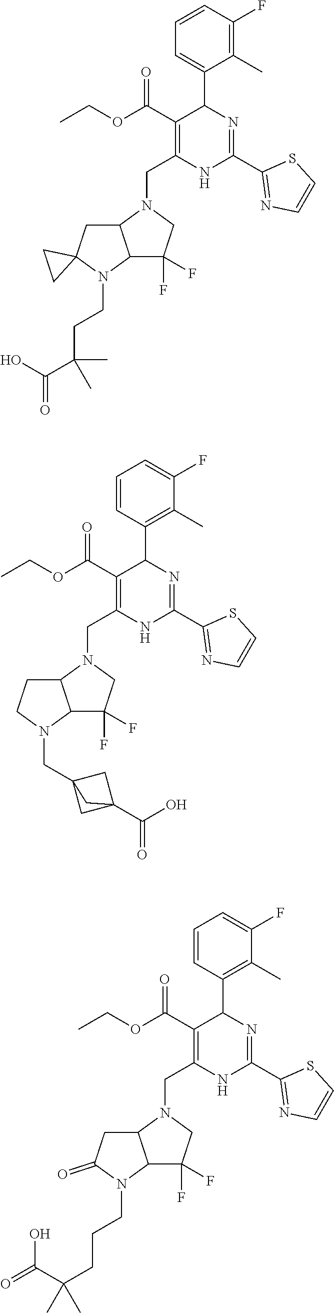 [SMILES: CCOC(=O)C1=C(CN2CC(F)(F)C3C2CC2(CC2)N3CCC(C)(C)C(=O)O)NC(c2nccs2)=NC1c1cccc(F)c1C ; CCOC(=O)C1=C(CN2CC(F)(F)C3C2CCN3CC23CC(C(=O)O)(C2)C3)NC(c2nccs2)=NC1c1cccc(F)c1C ; CCOC(=O)C1=C(CN2CC(F)(F)C3C2CC(=O)N3CCCC(C)(C)C(=O)O)NC(c2nccs2)=NC1c1cccc(F)c1C] 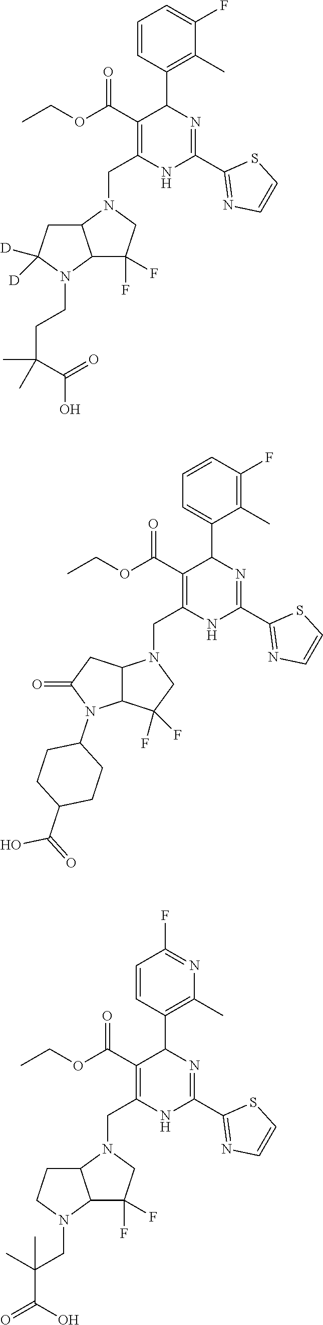 [SMILES: [2H]C1([2H])CC2C(N1CCC(C)(C)C(=O)O)C(F)(F)CN2CC1=C(C(=O)OCC)C(c2cccc(F)c2C)N=C(c2nccs2)N1 ; CCOC(=O)C1=C(CN2CC(F)(F)C3C2CC(=O)N3C2CCC(C(=O)O)CC2)NC(c2nccs2)=NC1c1cccc(F)c1C ; CCOC(=O)C1=C(CN2CC(F)(F)C3C2CCN3CC(C)(C)C(=O)O)NC(c2nccs2)=NC1c1ccc(F)nc1C] 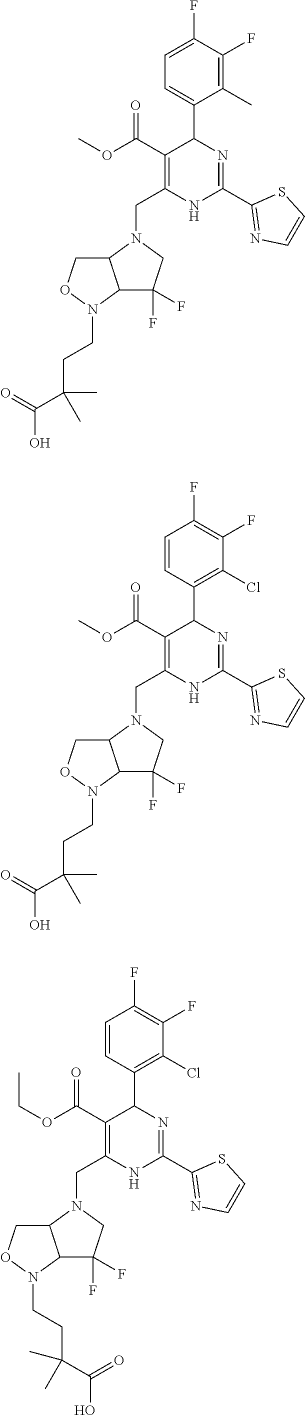 [SMILES: COC(=O)C1=C(CN2CC(F)(F)C3C2CON3CCC(C)(C)C(=O)O)NC(c2nccs2)=NC1c1ccc(F)c(F)c1C ; COC(=O)C1=C(CN2CC(F)(F)C3C2CON3CCC(C)(C)C(=O)O)NC(c2nccs2)=NC1c1ccc(F)c(F)c1Cl ; CCOC(=O)C1=C(CN2CC(F)(F)C3C2CON3CCC(C)(C)C(=O)O)NC(c2nccs2)=NC1c1ccc(F)c(F)c1Cl] 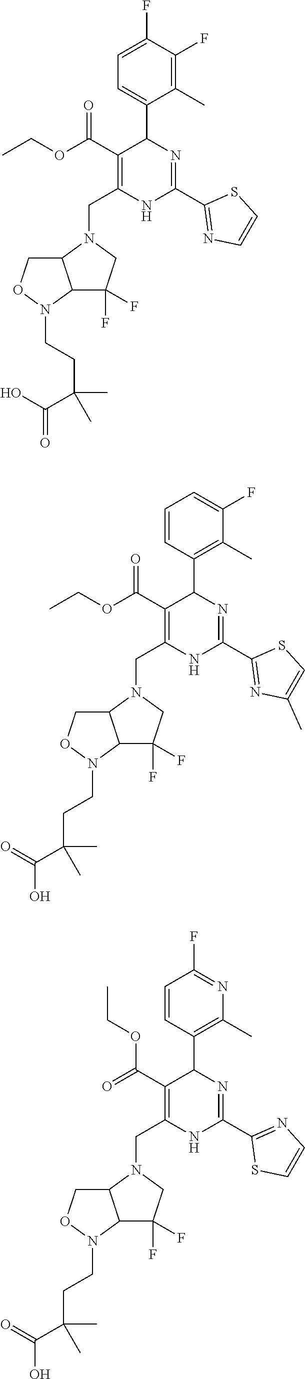 [SMILES: CCOC(=O)C1=C(CN2CC(F)(F)C3C2CON3CCC(C)(C)C(=O)O)NC(c2nccs2)=NC1c1ccc(F)c(F)c1C ; CCOC(=O)C1=C(CN2CC(F)(F)C3C2CON3CCC(C)(C)C(=O)O)NC(c2nc(C)cs2)=NC1c1cccc(F)c1C ; CCOC(=O)C1=C(CN2CC(F)(F)C3C2CON3CCC(C)(C)C(=O)O)NC(c2nccs2)=NC1c1ccc(F)nc1C] 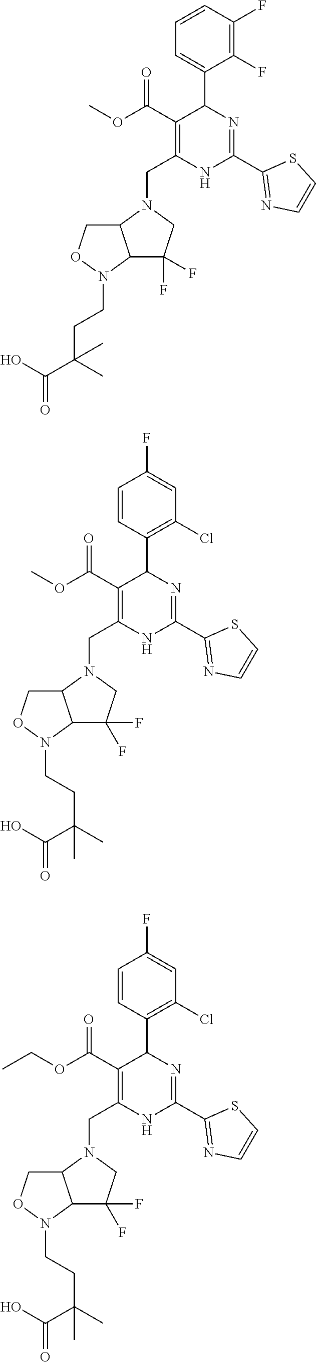 [SMILES: COC(=O)C1=C(CN2CC(F)(F)C3C2CON3CCC(C)(C)C(=O)O)NC(c2nccs2)=NC1c1cccc(F)c1F ; COC(=O)C1=C(CN2CC(F)(F)C3C2CON3CCC(C)(C)C(=O)O)NC(c2nccs2)=NC1c1ccc(F)cc1Cl ; CCOC(=O)C1=C(CN2CC(F)(F)C3C2CON3CCC(C)(C)C(=O)O)NC(c2nccs2)=NC1c1ccc(F)cc1Cl] 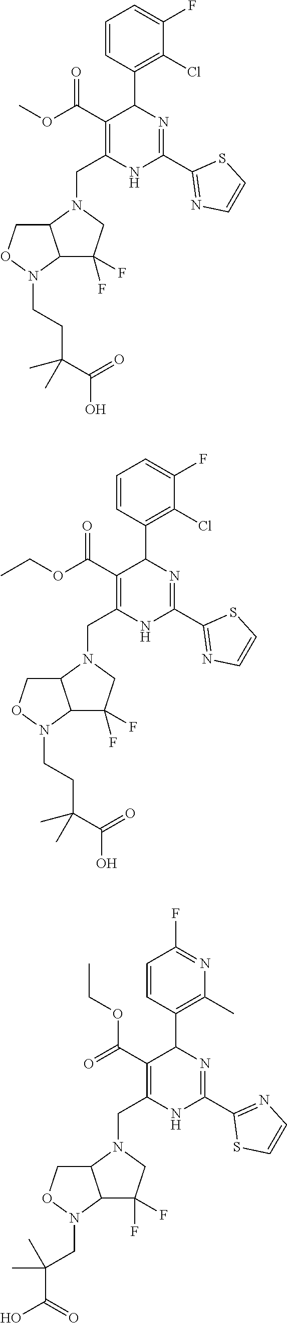 [SMILES: COC(=O)C1=C(CN2CC(F)(F)C3C2CON3CCC(C)(C)C(=O)O)NC(c2nccs2)=NC1c1cccc(F)c1Cl ; CCOC(=O)C1=C(CN2CC(F)(F)C3C2CON3CCC(C)(C)C(=O)O)NC(c2nccs2)=NC1c1cccc(F)c1Cl ; CCOC(=O)C1=C(CN2CC(F)(F)C3C2CON3CC(C)(C)C(=O)O)NC(c2nccs2)=NC1c1ccc(F)nc1C] 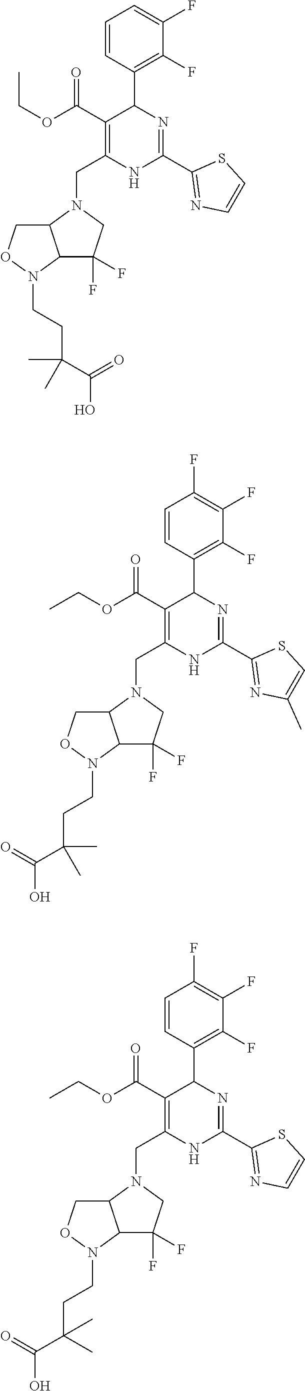 [SMILES: CCOC(=O)C1=C(CN2CC(F)(F)C3C2CON3CCC(C)(C)C(=O)O)NC(c2nccs2)=NC1c1cccc(F)c1F ; CCOC(=O)C1=C(CN2CC(F)(F)C3C2CON3CCC(C)(C)C(=O)O)NC(c2nc(C)cs2)=NC1c1ccc(F)c(F)c1F ; CCOC(=O)C1=C(CN2CC(F)(F)C3C2CON3CCC(C)(C)C(=O)O)NC(c2nccs2)=NC1c1ccc(F)c(F)c1F] 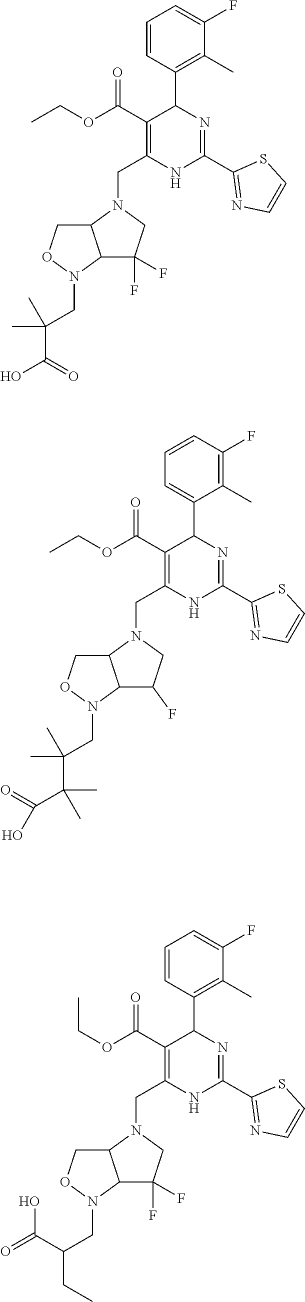 [SMILES: CCOC(=O)C1=C(CN2CC(F)(F)C3C2CON3CC(C)(C)C(=O)O)NC(c2nccs2)=NC1c1cccc(F)c1C ; CCOC(=O)C1=C(CN2CC(F)C3C2CON3CC(C)(C)C(C)(C)C(=O)O)NC(c2nccs2)=NC1c1cccc(F)c1C ; CCOC(=O)C1=C(CN2CC(F)(F)C3C2CON3CC(CC)C(=O)O)NC(c2nccs2)=NC1c1cccc(F)c1C] 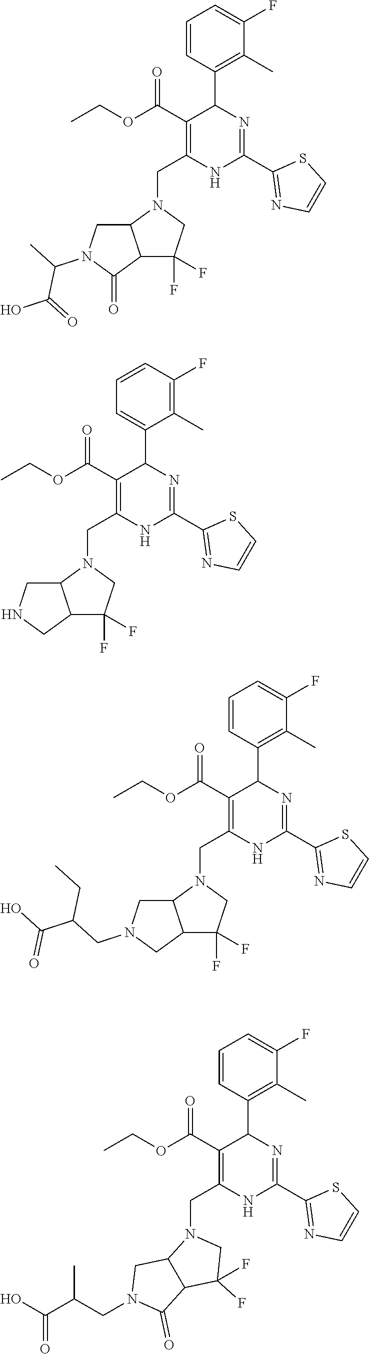 [SMILES: CCOC(=O)C1=C(CN2CC(F)(F)C3C(=O)N(C(C)C(=O)O)CC32)NC(c2nccs2)=NC1c1cccc(F)c1C ; CCOC(=O)C1=C(CN2CC(F)(F)C3CNCC32)NC(c2nccs2)=NC1c1cccc(F)c1C ; CCOC(=O)C1=C(CN2CC(F)(F)C3CN(CC(CC)C(=O)O)CC32)NC(c2nccs2)=NC1c1cccc(F)c1C ; CCOC(=O)C1=C(CN2CC(F)(F)C3C(=O)N(CC(C)C(=O)O)CC32)NC(c2nccs2)=NC1c1cccc(F)c1C] 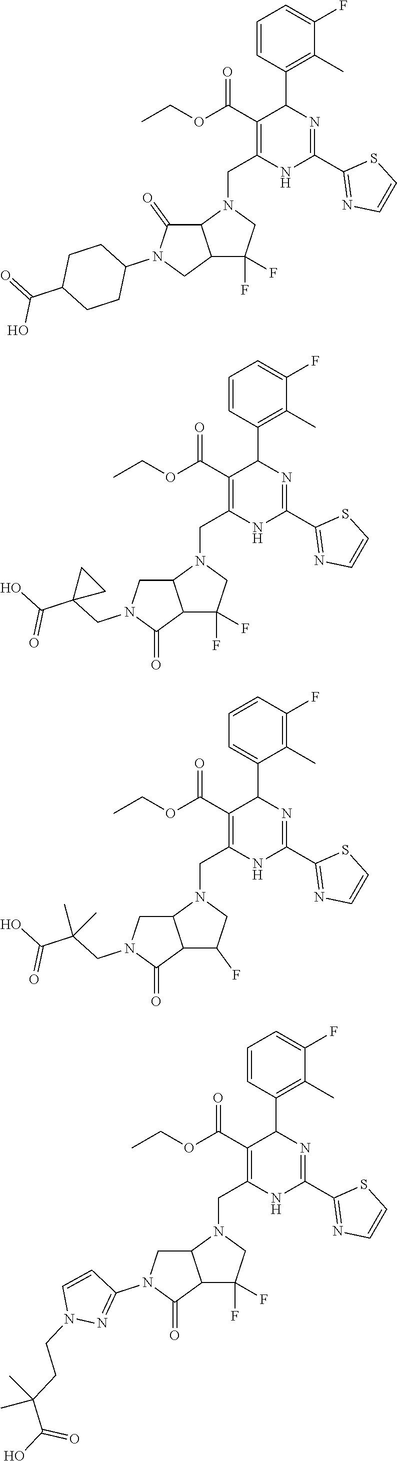 [SMILES: CCOC(=O)C1=C(CN2CC(F)(F)C3CN(C4CCC(C(=O)O)CC4)C(=O)C32)NC(c2nccs2)=NC1c1cccc(F)c1C ; CCOC(=O)C1=C(CN2CC(F)(F)C3C(=O)N(CC4(C(=O)O)CC4)CC32)NC(c2nccs2)=NC1c1cccc(F)c1C ; CCOC(=O)C1=C(CN2CC(F)C3C(=O)N(CC(C)(C)C(=O)O)CC32)NC(c2nccs2)=NC1c1cccc(F)c1C ; CCOC(=O)C1=C(CN2CC(F)(F)C3C(=O)N(c4ccn(CCC(C)(C)C(=O)O)n4)CC32)NC(c2nccs2)=NC1c1cccc(F)c1C] 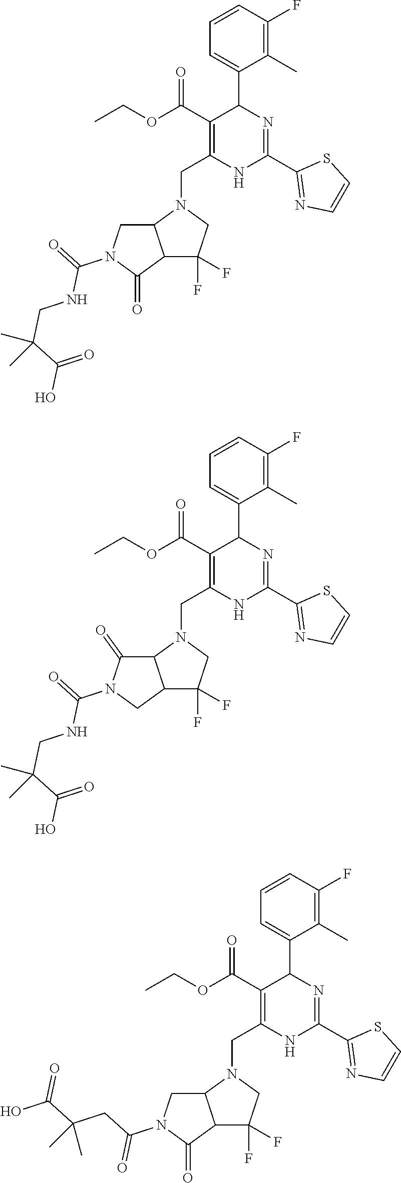 [SMILES: CCOC(=O)C1=C(CN2CC(F)(F)C3C(=O)N(C(=O)NCC(C)(C)C(=O)O)CC32)NC(c2nccs2)=NC1c1cccc(F)c1C ; CCOC(=O)C1=C(CN2CC(F)(F)C3CN(C(=O)NCC(C)(C)C(=O)O)C(=O)C32)NC(c2nccs2)=NC1c1cccc(F)c1C ; CCOC(=O)C1=C(CN2CC(F)(F)C3C(=O)N(C(=O)CC(C)(C)C(=O)O)CC32)NC(c2nccs2)=NC1c1cccc(F)c1C] 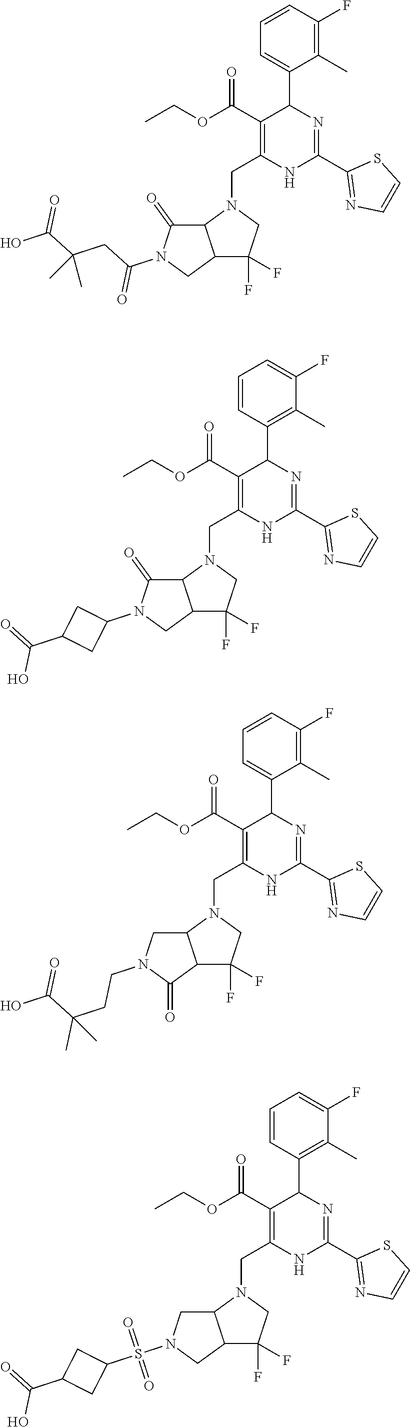 [SMILES: CCOC(=O)C1=C(CN2CC(F)(F)C3CN(C(=O)CC(C)(C)C(=O)O)C(=O)C32)NC(c2nccs2)=NC1c1cccc(F)c1C ; CCOC(=O)C1=C(CN2CC(F)(F)C3CN(C4CC(C(=O)O)C4)C(=O)C32)NC(c2nccs2)=NC1c1cccc(F)c1C ; CCOC(=O)C1=C(CN2CC(F)(F)C3C(=O)N(CCC(C)(C)C(=O)O)CC32)NC(c2nccs2)=NC1c1cccc(F)c1C ; CCOC(=O)C1=C(CN2CC(F)(F)C3CN(S(=O)(=O)C4CC(C(=O)O)C4)CC32)NC(c2nccs2)=NC1c1cccc(F)c1C] 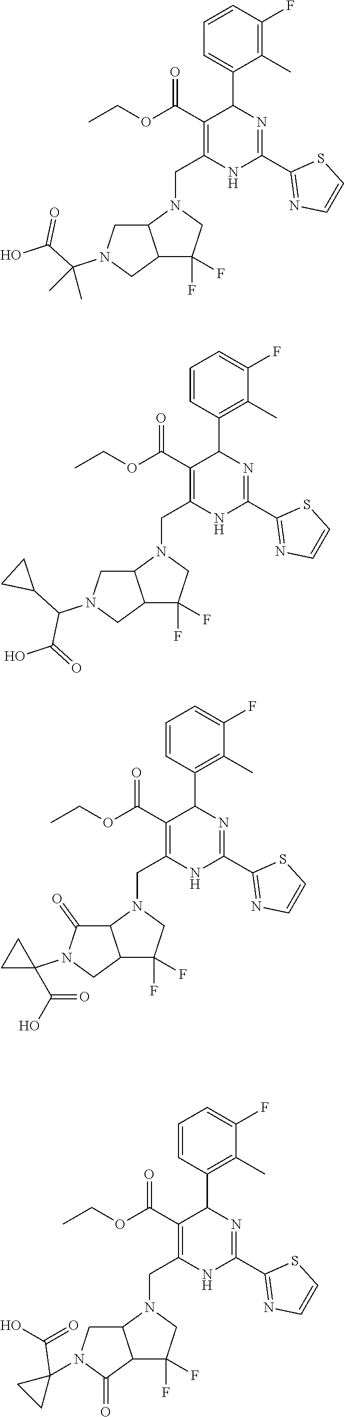 [SMILES: CCOC(=O)C1=C(CN2CC(F)(F)C3CN(C(C)(C)C(=O)O)CC32)NC(c2nccs2)=NC1c1cccc(F)c1C ; CCOC(=O)C1=C(CN2CC(F)(F)C3CN(C(C(=O)O)C4CC4)CC32)NC(c2nccs2)=NC1c1cccc(F)c1C ; CCOC(=O)C1=C(CN2CC(F)(F)C3CN(C4(C(=O)O)CC4)C(=O)C32)NC(c2nccs2)=NC1c1cccc(F)c1C ; CCOC(=O)C1=C(CN2CC(F)(F)C3C(=O)N(C4(C(=O)O)CC4)CC32)NC(c2nccs2)=NC1c1cccc(F)c1C] 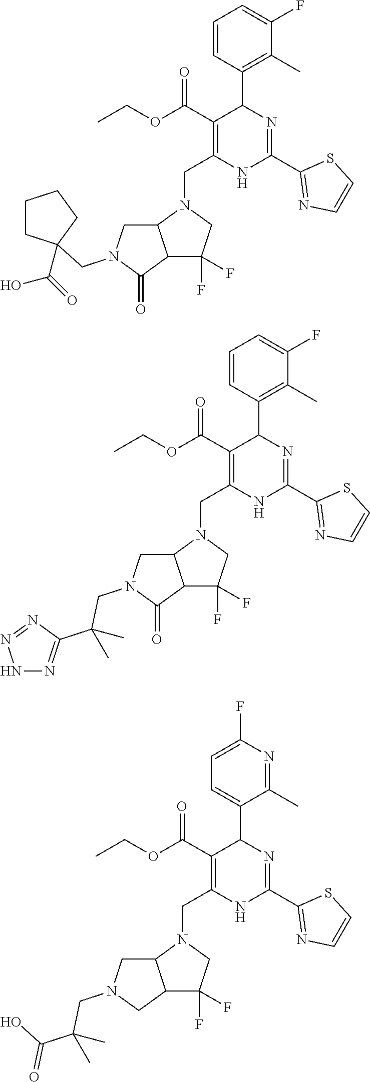 [SMILES: CCOC(=O)C1=C(CN2CC(F)(F)C3C(=O)N(CC4(C(=O)O)CCCC4)CC32)NC(c2nccs2)=NC1c1cccc(F)c1C ; CCOC(=O)C1=C(CN2CC(F)(F)C3C(=O)N(CC(C)(C)c4nn[nH]n4)CC32)NC(c2nccs2)=NC1c1cccc(F)c1C ; CCOC(=O)C1=C(CN2CC(F)(F)C3CN(CC(C)(C)C(=O)O)CC32)NC(c2nccs2)=NC1c1ccc(F)nc1C] 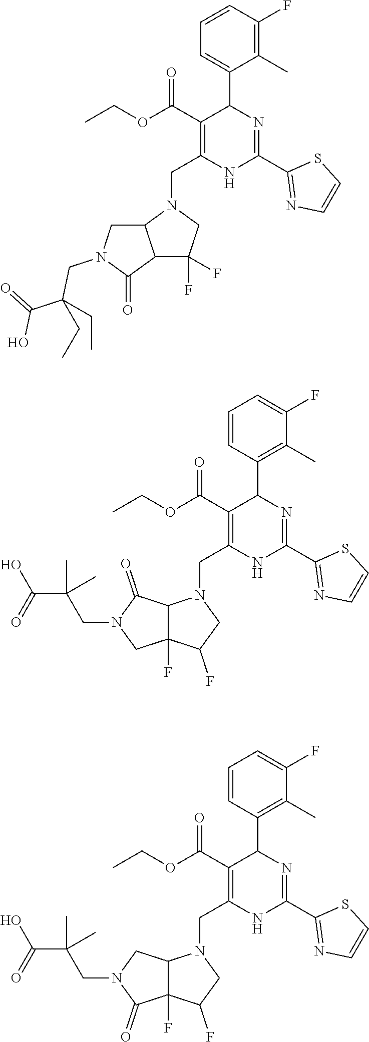 [SMILES: CCOC(=O)C1=C(CN2CC(F)(F)C3C(=O)N(CC(CC)(CC)C(=O)O)CC32)NC(c2nccs2)=NC1c1cccc(F)c1C ; CCOC(=O)C1=C(CN2CC(F)C3(F)CN(CC(C)(C)C(=O)O)C(=O)C23)NC(c2nccs2)=NC1c1cccc(F)c1C ; CCOC(=O)C1=C(CN2CC(F)C3(F)C(=O)N(CC(C)(C)C(=O)O)CC23)NC(c2nccs2)=NC1c1cccc(F)c1C] 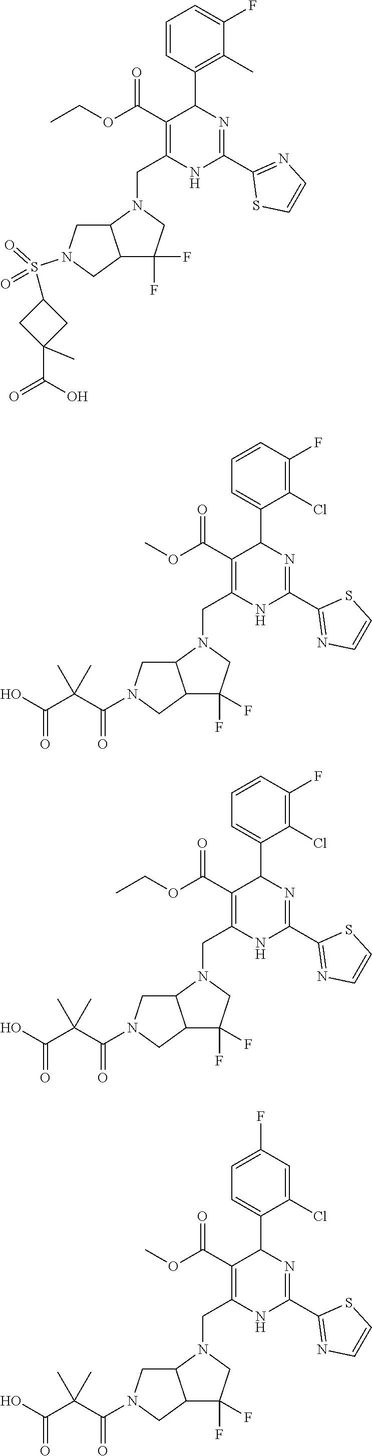 [SMILES: CCOC(=O)C1=C(CN2CC(F)(F)C3CN(S(=O)(=O)C4CC(C)(C(=O)O)C4)CC32)NC(c2nccs2)=NC1c1cccc(F)c1C ; COC(=O)C1=C(CN2CC(F)(F)C3CN(C(=O)C(C)(C)C(=O)O)CC32)NC(c2nccs2)=NC1c1cccc(F)c1Cl ; CCOC(=O)C1=C(CN2CC(F)(F)C3CN(C(=O)C(C)(C)C(=O)O)CC32)NC(c2nccs2)=NC1c1cccc(F)c1Cl ; COC(=O)C1=C(CN2CC(F)(F)C3CN(C(=O)C(C)(C)C(=O)O)CC32)NC(c2nccs2)=NC1c1ccc(F)cc1Cl] 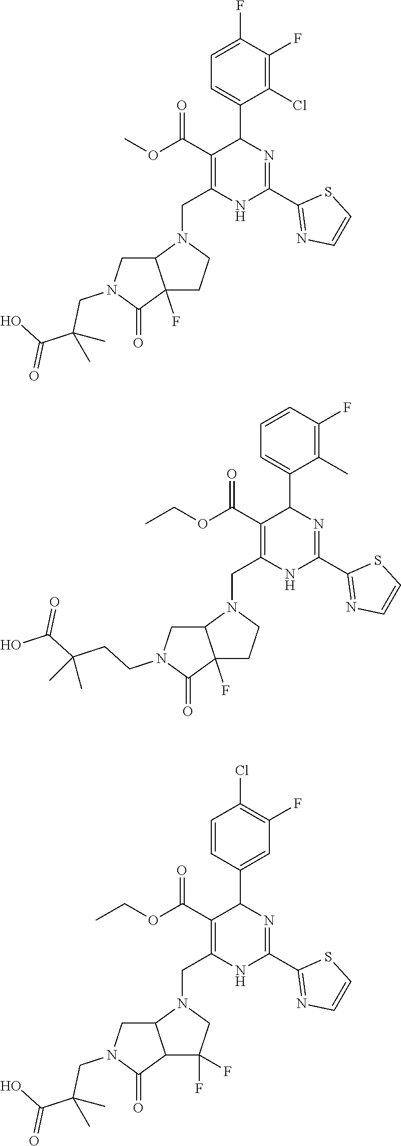 [SMILES: COC(=O)C1=C(CN2CCC3(F)C(=O)N(CC(C)(C)C(=O)O)CC23)NC(c2nccs2)=NC1c1ccc(F)c(F)c1Cl ; CCOC(=O)C1=C(CN2CCC3(F)C(=O)N(CCC(C)(C)C(=O)O)CC23)NC(c2nccs2)=NC1c1cccc(F)c1C ; CCOC(=O)C1=C(CN2CC(F)(F)C3C(=O)N(CC(C)(C)C(=O)O)CC32)NC(c2nccs2)=NC1c1ccc(Cl)c(F)c1]
or a deuterated form, stereoisomer or tautomer thereof, or a pharmaceutically acceptable salt or a solvate thereof.

12. A pharmaceutical composition comprising the compound of claim 1 and at least one pharmaceutically acceptable carrier.

13. (canceled)

14. A method of preventing or treating an HBV infection or of an HBV-induced disease in a mammal in need thereof, the method comprising administering to the mammal the pharmaceutical composition according to claim 12.

15. A product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound of claim 1.

16. A process for producing a compound of Formula (I) according to claim 1, the process comprising:
reacting a compound of Formula (I-2) 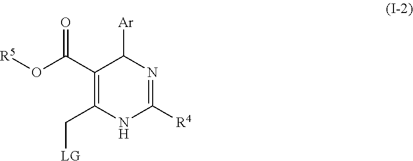
wherein Ar, R¹-R⁵ are as defined in claim 1, and LG represents a suitable leaving group; with a compound of Formula (V) 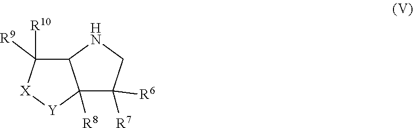
wherein R⁶-R¹⁰, X and Y are as defined in claim 1;
under suitable nucleophilic substitution conditions.

17. A method of treating an HBV infection in an individual in need thereof, comprising administering to the individual a therapeutically effective amount of the pharmaceutical composition of claim 12.

18. A process for preparing the pharmaceutical composition of claim 12, comprising mixing at least one pharmaceutically acceptable carrier with a therapeutically effective amount of a compound of Formula (I).

19. A method of preventing or treating an HBV infection or of an HBV-induced disease in mammal in need thereof, the method comprising administering to the mammal the compound according to claim 1.

20. A product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the pharmaceutical composition of claim 12.

21. A method of treating an HBV infection in an individual in need thereof, comprising administering to the individual a therapeutically effective amount of the compound according to claim 1.


SEQUENCE LISTING (3 sequences)

SEQ ID NO: 1 - DNA, 20 bp, Artificial Sequence
  primer
gtgtctgcggcgttttatca

SEQ ID NO: 2 - DNA, 21 bp, Artificial Sequence
  primer
gacaaacgggcaacatacctt

SEQ ID NO: 3 - DNA, 28 bp, Artificial Sequence
  probe
cctctkcatcctgctgctatgcctcatc